(19) United States
(12) Patent Application Publication
(10) Pub. No.: US 2025/0042951 A1
(43) Pub. Date: Feb. 6, 2025

(54) PRE-FUSION RSV F ANTIGENS

(71) Applicant: GLAXOSMITHKLINE BIOLOGICALS SA, Rixensart (BE)

(72) Inventors: Kurt SWANSON, Newton, MA (US); Andrea CARFI, Cambridge, MA (US)

(21) Appl. No.: 18/895,059

(22) Filed: Sep. 24, 2024

Related U.S. Application Data
(63) Continuation of application No. 17/848,247, filed on Jun. 23, 2022.
Application No. 17/848,247 is a continuation of application No. 17/314,649, filed on May 7, 2021, now abandoned.
Application No. 17/314,649 is a continuation of application No. 15/789,074, filed on Oct. 20, 2017, now abandoned.
Application No. 15/789,074 is a continuation of application No. 14/117,588, filed on May 16, 2014, now abandoned.
(60) Provisional application No. 61/486,005, filed on May 13, 2011.

(51) Int. Cl. C07K 14/005; A61K 39/00; A61K 39/12; A61K 39/155

(52) U.S. Cl. CPC C07K 14/005; A61K 39/12; A61K 39/155; A61K 2039/53; C07K 2319/735; C12N 2760/18522; C12N 2760/18534

(57) ABSTRACT

The invention relates to pre-fusion RSV F protein and polypeptides that contain one or more amino acid mutations that stabilize the pre-fusion conformation or destabilize the post-fusion conformation. The invention also relates to methods for inducing an immune response to pre-fusion RSV F.

### RELATED APPLICATION

This application is a Continuation of copending application Ser. No. 17/314,649, filed on May 7, 2021, which is Continuation of copending application Ser. No. 15/789,074, filed on Oct. 20, 2017, which is a Continuation of application Ser. No. 14/117,588, filed on May 16, 2014, which is the National Phase under 35 U.S.C. § 371 of International Application No. PCT/US2012/37773, filed on May 14, 2012, which claims the benefit under 35 U.S.C. § 119(e) to U.S. Provisional Application No. 61/486,005, filed on May 13, 2011, all of which are hereby expressly incorporated by reference into the present application.

### SEQUENCE LISTING

The instant application contains a Sequence Listing which was previously submitted in ASCII format via EFS-Web on Oct. 20, 2017, and transferred to the present application upon filing, and is hereby incorporated by reference in its entirety. Said ASCII copy, created on May 11, 2012, is named Pat05457.txt and is 98,385 bytes in size.

### BACKGROUND OF THE INVENTION

Respiratory syncytial virus (RSV) is an enveloped non-segmented negative-strand RNA virus in the family Paramyxoviridae, genus Pneumovirus. It is the most common cause of bronchiolitis and pneumonia among children in their first year of life. RSV also causes repeated infections including severe lower respiratory tract disease, which may occur at any age, especially among the elderly or those with compromised cardiac, pulmonary, or immune systems.

To infect a host cell, paramyxoviruses such as RSV, like other enveloped viruses such as influenza virus and HIV, require fusion of the viral membrane with a host cell's membrane. For RSV the conserved fusion protein (RSV F) fuses the viral and cellular membranes by coupling irreversible protein refolding with juxtaposition of the membranes. In current models, based on paramyxovirus studies, the RSV F protein initially folds into a “pre-fusion” conformation. During cell entry, the pre-fusion conformation undergoes refolding and conformational changes to its “post-fusion” conformation.

The RSV F protein is translated from mRNA into an approximately 574 amino acid protein designated F₀. Post-translational processing of F₀ includes removal of an N-terminal signal peptide by a signal peptidase in the endoplasmic reticulum. F₀ is also cleaved at two sites (109/110 and 136/137) by cellular proteases (in particular furin) in the trans-Golgi. This cleavage results in the removal of a short intervening sequence and generates two subunits designated F₁ (˜50 kDa; C-terminal; approximately residues 137-574) and F₂ (˜20 kDa; N-terminal; approximately residues 1-109) that remain associated with each other. F₁ contains a hydrophobic fusion peptide at its N-terminus and also two amphipathic heptad-repeat regions (HRA and HRB). HRA is near the fusion peptide and HRB is near the transmembrane domain. Three F₁-F₂ heterodimers are assembled as homotrimers of F₁-F₂ on the surface of the virion.

A vaccine against RSV infection is not currently available, but is desired. Vaccine candidates based on the chief RSV neutralization antigen, the F glycoprotein, have foundered due to problems with stability, purity, reproducibility, and potency. Crystal structures of related parainfluenza F glycoproteins have revealed a large conformational change between the pre-fusion and post-fusion states. The magnitude of the rearrangement suggested that post-fusion F antigens would not efficiently elicit neutralizing antibodies, which presumably must bind epitopes exposed on the pre-fusion conformation. Accordingly, efforts to produce a vaccine against RSV have focused on developing subunit vaccines that contain pre-fusion forms of RSV F. (See, e.g., WO 2010/149745, WO 2010/149743, WO 2009/079796) This focus on pre-fusion forms of RSV F has been corroborated by available models of RSV F.

Pre-fusion F is a “metastable” structure that readily rearranges into the lower energy post-fusion state, which then aggregates due to exposure of a hydrophobic fusion peptide (Begona Ruiz-Arguello, M. et al. Virology 298, 317-326 (2002) (142)). Large structural differences between the lollipop-shaped pre-fusion F trimer and the crutch-shaped post-fusion F trimer are apparent even at the resolution of electron microscopy of negatively stained specimens, suggesting that pre-fusion and post-fusion F may be antigenically distinct (Calder, L. J. et al. Virology 271, 122-131 (2000) (143)). To prevent viral entry, F-specific neutralizing antibodies presumably must bind the pre-fusion conformation of F on the virion, before the viral envelope fuses with a cellular membrane. However, efforts to generate a soluble, stabilized pre-fusion F subunit antigen have not yet yielded candidates suitable for testing in humans. Furthermore, analysis of a Motavizumab-peptide complex and homology modeling suggested that the dominant neutralizing epitope recognized by Palivizumab and Motavizumab might be buried in trimeric F, requiring at least a local dissociation for surface exposure to allow antibody binding (McLellan, J. S. et al. Nat Struct Mol Biol 17, 248-250 (2010)). There is a need for improved RSV F protein compositions and methods for making RSV F protein compositions.

### SUMMARY OF THE INVENTION

The invention relates to pre-fusion respiratory syncytial virus (RSV) F polypeptides and pre-fusion chimeric F polypeptides.

In some aspects, the pre-fusion respiratory syncytial virus (RSV) F polypeptide comprises at least two introduced cysteine residues that are in close proximity to one another, and form a disulfide bond that stabilizes the pre-fusion RSV F polypeptide. In particular embodiments, the HRB region contains an introduced cysteine residue and the DI and/or DII region contain an introduced cysteine residue, and a disulfide bond is formed between the introduced cysteine residue in the HRB region and the introduced cysteine residue in the D1 or DII region. In other embodiments, the HRA region contains an introduced cysteine residue and the DIII region contains an introduced cysteine residue, and a disulfide bond is formed between the introduced cysteine residue in the HRA region and the introduced cysteine residue in the III region. In other embodiments, the HRA region contains at least 2 introduced cysteine residues, and a disulfide bond is formed between the introduced cysteine residues in the HRA region.

In other aspects, the pre-fusion respiratory syncytial virus (RSV) F polypeptide comprises a post-fusion modification selected from the group consisting of deletion of the HRA helix, deletion of the HRB helix, introduction of point mutations, addition of glycosylation sites and combinations thereof, wherein said post-fusion modification destabilizes the post-fusion conformation. In some embodiments, the destabilizing post-fusion modification is deletion of the HRB helix, in whole or in part. If desired, the destabilizing post-fusion modification can further comprise deletion of the fusion peptide, in whole or in part. In other embodiments, the destabilizing post-fusion modification includes addition of a glycosylation site, such as glycosylation on a residue selected from the group consisting of position 173, position 175 and position 184.

In another aspect, the invention is a pre-fusion respiratory syncytial virus (RSV) F protein comprising three RSV F monomers, wherein at least two of the monomers contain an introduced cysteine residue, the introduced cysteine residues are in close proximity to one another and form a disulfide bond that stabilizes the pre-fusion RSV F protein.

In another aspect, the invention is a chimeric pre-fusion F protein comprising a stabilized F protein from a virus other than RSV, such as parainfluenza virus F polypeptide or a metapneumovirus virus F polypeptide, that contains one or more neutralizing epitope of RSV F. Suitable neutralizing epitopes can be selected from the group consisting of the epitopes that are recognized by motavizumab, palivizumab, mAb 11, mAb 151, mAb 1129, mAb 1153, mAb 1200, mAb 1214, mAb 1237, mAb 47F, mAb 7C2, mAb B4, Fab 19, mAb AK13A2, mAb 7.936, mAb 9.936, mAb 19, mAb 20, mAb 101F and combinations thereof.

The invention relates to methods for inducing an anti-respiratory syncytial virus (RSV) immune response in a subject, comprising administering to the subject an effective amount of an immunogenic composition comprising a pre-fusion RSV F protein or a pre-fusion chimeric F protein. Preferably, the induced immune response is characterized by neutralizing antibodies to RSV and/or protective immunity against RSV.

In particular aspects, the invention relates to a method for inducing or raising neutralizing anti-respiratory syncytial virus (RSV) F protein antibodies in a subject, comprising administering to the subject an effective amount of an immunogenic composition comprising a pre-fusion RSV F protein or a pre-fusion chimeric F protein.

In particular aspects, the invention relates to a method for inducing or raising protective immunity against respiratory syncytial virus (RSV) in a subject, comprising administering to the subject an effective amount of an immunogenic composition comprising a pre-fusion RSV F protein or a pre-fusion chimeric F protein.

In particular aspects, the invention relates to immunogenic compositions comprising a pre-fusion respiratory syncytial virus (RSV) F protein or a pre-fusion chimeric F protein.

The pre-fusion RSV F protein that is used in the invention can be full length or truncated, such as a soluble ectodomain that lacks the cytoplasmic and transmembrane domains. The pre-fusion RSV F protein, e.g., full length or soluble ectodomain, may comprise functional furin cleavage sites at positions 109/110 and 136/137. In some preferred embodiments, that pre-fusion RSV F protein (e.g., full length or soluble ecto-domain) contains the amino acid sequence of the corresponding portion (e.g., ecto-domain) of a naturally occurring RSV F protein. In any of the aspects of the invention, the pre-fusion RSV F protein can be administered with or without an adjuvant as desired, and the immunogenic composition can comprise an adjuvant if desired.

### BRIEF DESCRIPTION OF THE DRAWINGS

FIG. 1 shows a structure-based sequence alignment of four F proteins, secondary assignment, and key features. The alignment of RSV (SEQ ID NO:33), Newcastle disease virus (NDV) (SEQ ID NO:34), PIV3 (SEQ ID NO:36) and PIV5 (SEQ ID NO:35) Fs was generated with ClustalW2 (http://www.ebi.ac.uk/Tools/msa/clustalw2/), adjusted manually based on structural superposition using Lsqkab from the CCP4 suite of programs and displayed using ESPript version 2.2 (http://espript.ibcp.fr/ESPript/ESPript/). Features of RSV F are indicated above the sequences; features of PIV3 F are indicated below the sequences. *CHO indicates RSV F glycosylation sites. Secondary structure elements are indicated, with arrows parallel to the sequences designating β-sheets, cylinders designating α-helices, “TT” designating turns, and coils designating 3₁₀ helices. The domain location of secondary structure symbols are indicated (DI, DII, DIII), except for RSV helices α5 and α6, which are labeled to indicate that they form the Motavizumab binding site and β20 and β21, which are labeled to indicate that they form the 101F binding site. Circled numbers (RSV) or triangled numbers (PIV3) designate residues that form disulfide bonds, with the same number for each partner in a disulfide-linked pair. The furin cleavage sites for RSV F and PIV3 F are indicated by vertical arrows labeled Fr. The RSV F p27 region released from the protein after furin cleavage is indicated by a black bar. The fusion peptides of RSV F and PIV3 F are labeled. The arrow in the fusion peptide indicates the first residue of the F₁ fragment in the fusion peptide deletion construct used in this study. Residues that are identical in all four proteins are indicated by shaded boxes. Peptides used to investigate neutralizing binding sites are in open boxes and resistance mutations are indicated by asterisks.

FIG. 2A-C shows electron microscopy and circular dichroism analysis of the RSV F post-fusion trimer. In FIG. 2A an electron micrograph of the RSV F protein shows a field of uniform crutch phenotypes consistent with the structure of post-fusion F proteins.

FIG. 2B shows a CD melting curve of the post-fusion RSV F trimer observed at 210 nm, the observed spectral minimum of the folded RSV F protein. The CD absorption, y-axis, is plotted against temperature, x-axis. FIG. 2C shows a CD spectra of the post-fusion RSV F trimer at 200 and 95° C. The spectra were recorded from 320 to 190 nm and show at both temperatures characteristic helical minima for a folded protein.

FIG. 3A-F shows representative electron densities of the crystallized RSV F protein. FIG. 3A is a side view of the original molecular replacement solution model, which contains the PIV3 post-fusion head in-frame with the 6-helix bundle of RSV F, shown in the initial electron density map (1π) calculated after iterative real-space NCS three-fold averaging, histogram matching, and solvent flattening with phase extension from 7.0 to 3.2 Å and no phase recombination. The head region fits poorly in the electron density. FIG. 3B Side view. The final model of RSV F shown in the averaged electron density map as described in FIG. 4A. FIG. 3C shows a top view of the RSV F protein structure shown in FIG. 4A. FIG. 3D shows a top view of B. Model and electron density depicted as in FIG. 4B. FIG. 3E is a close up of a representative averaged electron density (gray) with the final model in stick representation. FIG. 3F shows the same view as in FIG. 4E but with final 2mFo-dFc electron density map contoured at 1.5 σ.

FIG. 4A-C shows the RSV F ectodomain structure. 4A is a linear diagram. Listed residue numbers correspond to the N-terminus of each segment, the furin cleavage sites (arrow heads), and the C-terminus. DI-III, domains I-III; p27, excised peptide; FP, fusion peptide; HRA, B, and C, heptad repeats A, B, and C. 4B shows a ribbon representation of one subunit of the RSV F ectodomain trimer. Domains are labeled and shaded as in 4A, glycans are shown in black. 4C shows surface representation of the RSV F ectodomain trimer. The domains of one subunit are labeled and shaded as in 4A, the other two subunits are white and gray.

FIGS. 5A and B shows superposition of domains I and II of RSV F and PIV3 F. FIG. 5A shows a ribbon diagram of domain I from RSV and PIV3 superimposed by matching the common β-sheets. FIG. 5B shows a ribbon diagram of domain II from RSV F and PIV3 F superimposed based on common β-strands. The secondary structure elements of RSV F are labeled.

FIG. 6A-D illustrates a comparison between RSV and PIV3. FIG. 6A is a ribbon diagram of RSV F domain III. FIG. 6B shows a ribbon diagram of the PIV3 domain III oriented to match orientation shown in FIG. 6A. FIG. 6C shows the detail of the RSV and PIV3 (which are shaded differently) domain III helical bundles superimposed based on domain III β-sheets. FIG. 6D shows RSV and PIV3 F ectodomain trimers (shaded as in A and B) superimposed based on their six-helix bundles. The image on the left shows a ribbon diagram viewed perpendicular to the three-fold axis; the image on the right is a surface representation viewed along the three-fold axis from the top of the head.

FIGS. 7A and B illustrates the Motavizumab epitope. FIG. 7A is a superposition of the Motavizumab-binding helices, α5 and α6, from the RSV F post-fusion trimer and the peptide-Motavizumab complex (PDB code 3IXT). The post fusion trimer structure and the peptide-motavizumab complex structure are shaded differently. RSV residues bound by Motavizumab are shown in stick representation. Asterisks denote Palivizumab escape mutations. FIG. 7B shows a ribbon representation modeling a Motavizumab-RSV F post-fusion trimer complex. The V_{H} and V_{L} domains of the Fab are labeled; helices α5 and α6 from the RSV F structure and the peptide-Motavizumab structure are shaded differently; a glycan on RSV F is black; and the remainder of RSV F is white.

FIG. 8A-C illustrates RSV F conformational changes, antigenic structure and Palivizumab binding. FIG. 8A is a surface representation of the post-fusion structure. Antigenic sites A and C are outlined and labeled. Asterisks indicate residues selected in neutralization escape variants or forming contacts with an antibody in the determined structures of neutralizing antibody-peptide complexes. The HRA and HRB surfaces are shaded. FIG. 8B is a surface representation of a pre-fusion model, annotated as in A. FIG. 8C is a graph showing inhibition of Palivizumab binding to post-fusion RSV F by pooled sera from un-immunized mice or mice immunized with the RSV F antigen. Palivizumab binding (percentage of ELISA signal without competing sera) is plotted as a function of the dilution of competing pooled sera.

FIGS. 9A and B shows the exposure of the Motaviuzumab epitope in the post-fusion RSV F structure (A) and pre-fusion RSV F model (B). FIG. 9A shows Domain III of one subunit from the post-fusion structure shaded black and grey while the remaining parts of RSV F are in white. Structural elements that do not significantly change betweeen pre- and post-fusion are in black while HRA (labeled with arrow), which refolds in the transition from the pre- to post-fusion conformation, is lighter grey. Motavizumab epitopes on two subunits are also labeled. A third Motavizumab epitope is present on the trimer surface, but is not easily visible in this orientation. The Motavizumab epitope α5 and α6 helices are labeled on one example. FIG. 9B shows Domain III of the pre-fusion model shaded as in A. The fusion peptide region is shaded and labeled FP. The HRA region is broken into structural elements α1, α2, α3, β1 and β2; labeled and shaded grey for one subunit. In both the pre-fusion and post-fusion structures, the α5 and α6 helices of the Motavizumab epitope are surface exposed. However, in the pre-fusion model, the HRC loop may need to shift to accommodate antibody binding (as indicated by the arrow).

FIGS. 10A and B shows a model of neutralizing antibody 101F bound to the post-fusion RSV F trimer. FIG. 10A shows the peptide (residues 431-435) (SEQ ID NO:37) from the 101F Fab-peptide complex structure (PDB code 3O41²¹) superposed on equivalent residues of the RSV F structure (β-strand 20 to β-strand 21). FIG. 10B is a ribbon representation of a model of the 101F Fab bound to the RSV F post-fusion trimer. 101F Fab V_{H} and V_{L} domains are labeled; RSV F β-strand 20 and β-strand 21 and labeled as in A. The remaining parts of RSV F are in white. FIG. 10B discloses “IIKTF” as SEQ ID NO: 37.

FIGS. 11A-C show that residues within appropriate distances to form disulfides can be identified, based on the current model of RSV F pre-fusion, can be identified. FIG. 11A (Center), The pre-fusion RSV F model is shaded/colored to show structural features which are labeled (HRB, HRA, DIII). FIG. 11B (Left) is a zoom-in view of the packing of HRA on domain III in the pre-fusion model. The paired numbers indicate residues in close proximity which, if mutated to cysteines could form a disulfide bond. FIG. 11C (Right) is a zoom-in view of the packing of the HRB-stalk on domains I and III (white). The paired numbers indicate residues which, if mutated to cysteines could form a disulfide bond. Amino acid residues 165 and 296, and 56 and 164 are not at ideal distances to one another, in the pre-fusion model, to form disulfide bonds, but are in correct orientation and can form disulfides if the model is biased by the PIV5 structure, on which the model was built.

FIG. 12 shows a negative stained electron micrograph of the HRB-deleted RSV F construct (RSV F delHRB HIS, SEQ ID NO:28). Electron microscopy of the HRB-deleted RSV F construct demonstrated the RSV F protein formed rosettes, likely through the fusion peptide. The formation of rosettes through the fusion peptide is a feature of post-fusion RSV F rather than a predicted behavior of pre-fusion F proteins (Ruiz-Arguello et al. 2004 (142) and Connolly et al, 2006 (144)). This result shows the HRB-deleted RSV F construct does not appreciably stabilize the protein in the desired pre-fusion conformation.

FIG. 13 shows the structure of RSV F protein in which certain mutations are introduced to inhibit 6-helix bundle formation. Shown is the RSV F post-fusion structure in which the HRB helix has been removed and replaced with a hypothetical random coil (represented by the lines). The elongated HRA helix of the post-fusion RSV F is labeled. The numbers represent potential sites for introduced glycosylation sites or other mutations which interfere with formation of the 6-helix bundle characteristic of the post-fusion structure. A mutation on the HRA helix which interferes with HRB interaction would destabilize the post-fusion conformation, which in turn would cause the protein to remain in the favored pre-fusion conformation.

FIGS. 14A and B are western blots of cell lysates (14A, showing total expression) or media (14B, showing secreted protein) under boiled and reducing conditions using an anti-His tag antibody. The westerns show the expression of RSV-F proteins, and that proteins with engineered cysteine residues were expressed and contained intra-chain disulfide bonds. Cleavage of RSV F protein from F0 to F1/F2 and secretion from the cell is evidence of proper protein folding of the RSV F proteins. Migrations for uncleaved F0 and cleaved F1 are indicated. The key for gel lane labeling is shown above the blots. (A) Western blot of cell lysate indicating total protein expression. Each protein construct was well expressed by the cell. (B) Western blot of RSV F secreted into the media. The secreted protein was predominantly cleaved (F1) and the amount of secretion varied among proteins. The results of this analysis indicate that the T58C & V164C, K168C & V296C and M396C & F483C protein constructs were the best expressed/secreted protein constructs. R049: RSV-F fus del R429S I432T K433T S436F trunc (SEQ ID NO:39); R050: RSV-F HRA Disulfide2 I57C S190C trun (SEQ ID NO:40); R051: RSV-F HRA Disulfide3 T58C V164C trunc (SEQ ID NO:6); R052: RSV-F HRA Disulfide5 K168C V296C trunc (SEQ ID NO:8); R053: RSV-F fus del N262Y N268I K272M R429S I432T K433T S436F trunc (SEQ ID NO:41); R054 RSV-F HRA Disulfidel V56C V164C trunc (SEQ ID NO:4); R055 RSV-F HRA Disulfide4 N165C V296C trunc (SEQ ID NO: 7); R056 RSV-F HRB Disulfide M396C F483C trunc (SEQ ID NO:9).

FIGS. 15A-C show SEC analysis of RSV F intra-chain disulfide. Postfusion F rosettes and fusion peptide deleted RSV F trimers were used to develop an HPLC-SEC assay to differentiate between rosettes and trimers. (FIG. 15A) Fusion peptide-stabilized RSV F rosettes migrated with the void volume by SEC (retention time of 5 minutes on Bio-Sil 250 SEC column). Anti-HIS-tag western blotting confirmed that the protein was in the void volume peak. (FIG. 15B) Fusion peptide-deletion RSV F trimers migrated with an SEC retention time of approximately 6.5 minutes. Anti-HIS-tag western blot similarly confirmed that the protein was in the included volume trimer peak. Although the void peak is larger than the included volume peak, the anti-HIS western shows approximately equal amounts of RSV F are in the two peaks. (FIG. 15C) These data show that the RSV F T58C V164C construct had a population of cleaved RSV F that was in the form of monodispersed trimers rather than rosettes, suggesting that population of the protein construct was in the prefusion form.

FIGS. 16A-C show purification and analysis of RSV F protein constructs that contain engineered cysteins. FIG. 16A shows purification of the RSV F N165C/V296C construct. Columns are labeled for flow-through (FT), wash (W), elution (E) and resin (R) from a chelating purification. Unlike other protein constructs that contained introduced disulfide mutations and were expressed in insect cells, N165C/V296C secreted as a cleaved species, similar to its profile when expressed in mammalian cells. FIG. 16B shows a gel-shift analysis of the K168C/V296C and M396C/F483C RSV F protein constructs. On the left side of the standards are the two constructs run with boiling and reducing agent present. On the right side of the standards, the two constructs are run after boiling with no reducing agent (b/nr) or no boiling and no reducing agent (nb/nr). The western blot shows the protein is largely uncleaved, but that unexpectedly no inter-chain disulfide bonds were formed. K168C/V296C without boiling shifted to the trimer band, while non-boiled M396C/F483C ran as a monomer band. FIG. 16C shows a coomasie-stained gel of the K168C/V296C and M396C/F483C RSV F protein constructs with reducing and boiling. Approximately 50% of the material was cleaved.

FIG. 17(A) shows electron microscopy analysis of NDV F (prefusion) with the expected spherical heads for prefusion F, with a few rosette-like aggregates. FIG. 17(B) shows NDV prefusion F forms rod-like crystals. An isolated rod was analyzed and a dataset with ˜95% completion to ˜3.7 angstroms was recorded. FIG. 17(C) shows NDV prefusion F forms bipiramidal crystals (50×50×50 μm size).

FIGS. 18A-E show analysis of several RSV F protein constructs. FIG. 18A shows EM analysis of RSV F Del-HRB showing that 100% formed rosettes. The protein eluted from the SEC column in the void/rosette retention peak. FIG. 18(B) shows analysis/purification of the Del-HRB Del-FP RSV F construct. The protein was found in both the void and trimer retention peaks. FIG. 18C shows gel-analysis, which suggests that there was partially cleaved RSV F Del-HRB Del-FP present in both the void and trimer peaks. FIG. 18D shows EM of NDV F (prefusion), which shows the expected spherical heads for pre-fusion F with a few rosette-like aggregates. FIG. 18E shows that RSV F Del-HRB from the SEC trimer peak contains a heterogeneous mix of rosette-like structures, post-fusion crutches and pre-fusion head-like spherical species.

FIG. 19 shows SDS-PAGE analysis of chimeric RSV F/NDV and RSV F/PIV5 F protein constructs. The supernatant from cells transfected with one of the six constructs (1: RSV-F NDV HRB del fus trunc; 2: RSV-F NDV HRB trunc; 3: RSV-F NDV HRB2 del fus trunc; 4: RSV-F NDV HRB2 trunc; 5: RSV-F PIV5 HRB del fus trunc; 6: RSV-F PIV5 HRB trunc) was analyzed by SDS-PAGE. The constructs were engineered with or without (del fus) fusion peptide. The proteins either had an NDV HRB, an NDV HRB with an additional glycine residue as a linker (HRB2) or PIV5 HRB as indicated. For each construct, a cleaved F₁ protein was observed consistent with a processed F protein. One liter expressions of each protein was performed.

### DETAILED DESCRIPTION OF THE INVENTION

### Definitions

As used herein, “pre-fusion” RSV F proteins are RSV F proteins that share general structural architecture more similar to the PIV5 pre-fusion structure rather than the RSV F post-fusion structure. Pre-fusion RSV F proteins include the following characteristics: the HRA region is packed against domain III in the RSV F head region and/or the HRB region forms a trimer coil-coil stalk in proximity to domains I and II rather than associating with the HRA region in the context of the 6-helix bundle.

As used herein, “post-fusion conformation” of RSV F protein are RSV F proteins that share more general structural architecture with the RSV F post-fusion structure rather than the PIV5 pre-fusion structure. Post-fusion RSV F proteins include an HRA-HRB 6-helix bundle.

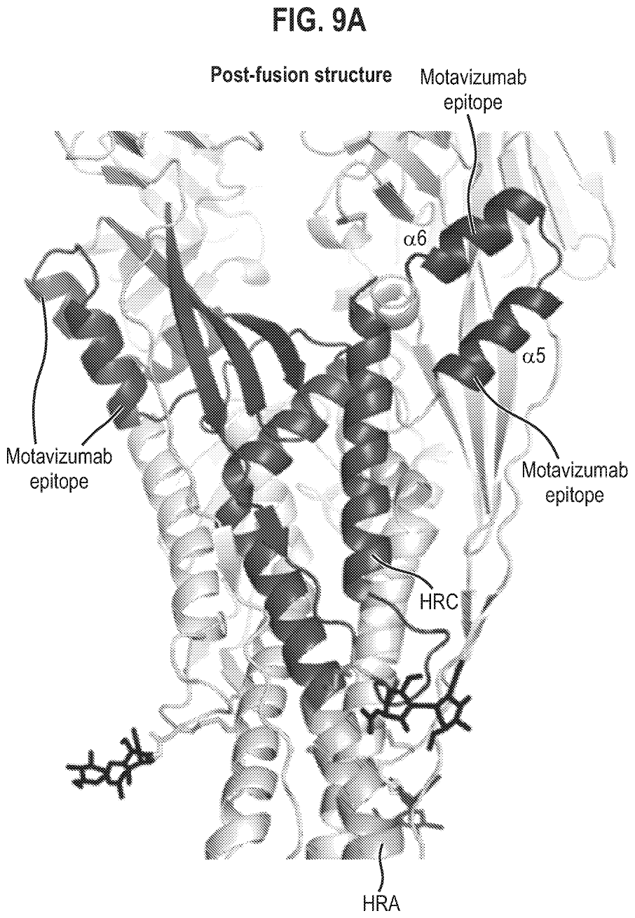

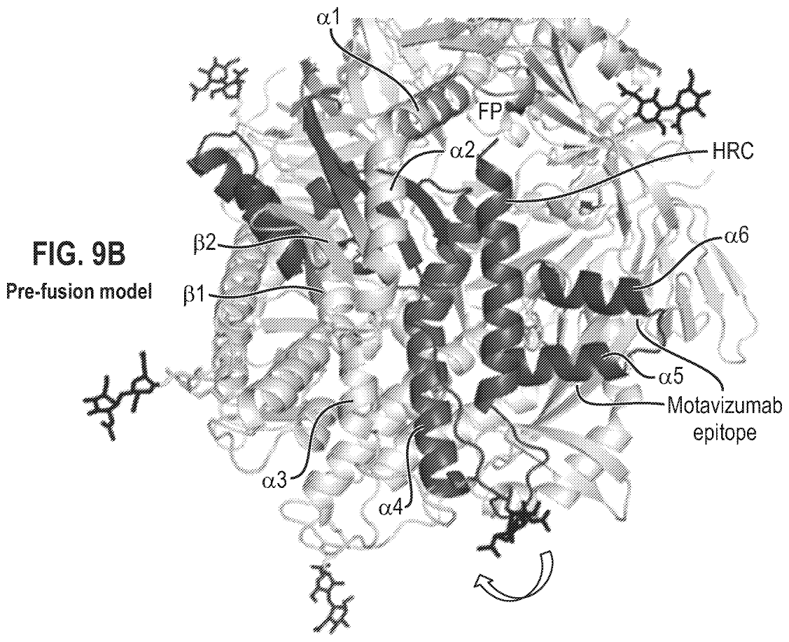

As used herein, HRA region in prefusion RSV F is approximately residues 137-239 of RSV F protein (SEQ ID NOS: 1 and 2) and comprises the fusion peptide, helix α1, helix α2, helix α3, helix α4, strand β1 and strand β2. See, FIG. 9B.

As used herein, the HRA helix in post fusion RSV F is formed by approximately residues 155-226 of RSV F protein (SEQ ID NOS: 1 and 2).

As used herein, the fusion peptide is defined by residues 137-154 of RSV F protein (SEQ ID NOS: 1 and 2).

As used herein, helix α1 in the prefusion RSV F HRA region is formed by approximately residues 145-157 of RSV F protein (SEQ ID NOS: 1 and 2).

As used herein, helix α2 in the prefusion RSV F HRA region is formed by approximately residues 158-167 of RSV F protein (SEQ ID NOS: 1 and 2).

As used herein, helix α3 in the prefusion RSV F HRA region is formed by approximately residues 168-176 of RSV F protein (SEQ ID NOS: 1 and 2).

As used herein, helix α4 in the prefusion RSV F HRA region is formed by approximately residues 194-212 of RSV F protein (SEQ ID NOS: 1 and 2).

As used herein, strand β1 in the prefusion RSV F HRA region is formed by approximately residues 177-184 of RSV F protein (SEQ ID NOS: 1 and 2).

As used herein, strand β2 in the prefusion RSV F HRA region is formed by approximately residues 185-193 of RSV F protein (SEQ ID NOS: 1 and 2).

As used herein, HRB region in RSV F is approximately residues 461-515 of RSV F protein (SEQ ID NOS: 1 and 2) and includes the HRB helix and the HRB linker

As used herein, the HRB helix in RSV F is formed by approximately residues 485-515 of RSV F protein (SEQ ID NOS: 1 and 2).

As used herein, the HRB linker in RSV F is formed by approximately residues 461-484 of RSV F protein (SEQ ID NOS: 1 and 2).

As used herein, domain I (DI) is formed by approximately residues 26-50 and 309-401 of RSV F protein (SEQ ID NOS: 1 and 2).

As used herein, domain II (DII) is formed by approximately residues 400-460 of RSV F protein (SEQ ID NOS: 1 and 2).

As used herein, domain III (DIII) is formed by approximately residues 51-98 and 206-308, or residues 51-308 of RSV F protein (SEQ ID NOS: 1 and 2).

As used herein, a “purified” protein or polypeptide is a protein or polypeptide which is recombinantly or synthetically produced, or produced by its natural host, and has been isolated from other components of the recombinant or synthetic production system or natural host such that the amount of the protein relative to other macromolecular components present in a composition is substantially higher than that present in a crude preparation. In general, a purified protein will be at least about 50% homogeneous and more preferably at least about 75%, at least about 80%, at least about 85%, at least about 90%, at least about 95% or substantially homogeneous.

As used herein, “substantially free of lipids and lipoproteins” refers to compositions, proteins and polypeptides that are at least about 95% free of lipids and lipoproteins on a mass basis when protein and/or polypeptide (e.g., RSV F polypeptide) purity is observed on an SDS PAGE gel and total protein content is measured using either UV280 absorption or BCA analysis, and lipid and lipoprotein content is determined using the Phospholipase C assay (Wako, code no. 433-36201).

As used herein, “close proximity” refers to a distance of not more than about 10 Å, not more than about 8 Å, not more than about 6 Å, not more than about 4 Å, or not more than about 2 Å. When two or more amino acid residues are in close proximity, the distance between the alpha carbons of the amino acid residues are is more than about 10 Å, not more than about 8 Å, not more than about 6 Å, not more than about 4 Å, or not more than about 2 Å.

Features of RSV F protein suitable for use in this invention are described herein with reference to particular amino acids that are identified by the position of the amino acid in the sequence of RSV F protein from the A2 strain (SEQ ID NO:1). RSV F proteins can have the amino acid sequence of the F protein from the A2 strain or any other desired strain. When the F protein from a strain other than the A2 strain is used, the amino acids of the F protein are to be numbered with reference to the numbering of the F protein from the A2 strain, with the insertion of gaps as needed. This can be achieved by aligning the sequence of any desired RSV F protein with the F protein of the strain A2. Sequence alignments are preferably produced using the algorithm disclosed by Corpet, Nucleic Acids Research, 1998, 16(22):10881-10890, using default parameters (Blossum 62 symbol comparison table, gap open penalty: 12, gap extension penalty: 2).

As described and exemplified herein, the 3.2 Å x-ray crystal structure of a post-fusion form of RSV F protein has been determined. A model of the pre-fusion form of RSV F protein was made by comparing the RSV F post-fusion x-ray crystal structure to the known structures of the pre- and post-fusion parainfluenza virus F proteins. This model of the pre-fusion form of RSV F reveals structural features that differ from those of prior models of pre-fusion RSV F and allows for rational structure-based design of stabilized pre-fusion forms of RSV F.

Accordingly, the invention relates to pre-fusion respiratory syncytial virus F (RSV F) polypeptides and/or proteins, and immunogenic compositions comprising pre-fusion RSV F polypeptides and/or proteins. The invention also relates to methods and use of pre-fusion RSV F polypeptides and/or proteins for inducing an immune response, and or by protective immunity against RSV. The invention also relates to nucleic acids that encode pre-fusion RSV F polypeptides and/or proteins.

Generally, the immunogenic compositions comprise pre-fusion RSV F polypeptides and/or proteins that elicit neutralizing antibodies. For example, antibodies that bind to the same epitopes as Palivizumab, Motavizumab and 101F.

Pre-fusion and post-fusion PIV F structures reveal en bloc shifts of domains and large rearrangements of HRA and HRB. In domain III of the pre-fusion PIV5 structure, HRA folds into three α-helices and two β-strands rather than the long post-fusion HRA helix (Yin et al, 2006). However, when pre-fusion and post-fusion conformations of individual PIV F domains are compared, the non-rearranging parts superimpose well. Superimposing post-fusion RSV F domains on their pre-fusion PIV5 F counterparts did not result in major clashes and positioned all the pairs of cysteines that form interdomain disulfide bonds in proximity to each other. The pre-fusion RSV F model obtained by thus combining information from the post-fusion RSV F x-ray crystal structure and the pre-fusion PIV5 F structure revealed a feature not apparent from prior homology models of pre-fusion RSV F based solely on the PIV5 pre-fusion structure (McLellan et al NSMB 2010 (141)). The helices of the Palivizumab/Motivizumab epitope are exposed on the surface of the pre-fusion RSV F trimer model, as they are on post-fusion RSV F trimer x-ray crystal structure (FIG. 9). In our pre-fusion RSV F model, the loop connecting P4 and HRC (part of domain III) could hinder access of Palivizumab or Motavizumab to their epitope. However, it is likely that the loop has sufficient flexibility to adopt an alternative conformation that permits antibody binding (FIG. 9B).

The pre-fusion model disclosed herein, which is based on the RSV F post-fusion x-ray crystal structure and the PIV5 prefusion structure (Yin et al, 2006 (145)), shows that the elongated HRA helix of the post-fusion RSV F (residues 137-212) folds into strands and helices similar to the PIV5 pre-fusion crystal structure. The fusion peptide of RSV F, residues 137-154, forms a coil and helix that is packed into the RSV F pre-fusion head. Four helices are formed; helix a1 is approximately residues 145-157, helix a2 is approximately residues 158-167, helix a3 is approximately residues 168-176 and helix a4 is approximately residues 194-212. Two strands are formed; strand b1 is approximately residues 177-184, strand b2 is approximately residues 185-193 (FIG. 9).

### Pre-Fusion Conformation

The invention includes pre-fusion RSV F polypeptides and proteins and immunogenic compositions that contain pre-fusion RSV F polypeptides and proteins. The RSV F protein and polypeptides can contain 1 or more amino acid replacements, deletions and/or additions that stabilize the pre-fusion conformation or destabilize the post-fusion conformation, for example, a pre-fusion RSV F stabilized with disulfide bonds, or a pre-fusion RSV F formed by destabilizing the post-fusion conformation.

### Stabilization Through Disulfide Bonds

The RSV F pre-fusion model may be used as a guide to select amino acid residues that are in close proximity to each other in the pre-fusion conformation and that are no longer in close proximity in the post-fusion conformation. Such amino acids may be mutated to cysteine residues to allow disulfide formation that stabilizes the prefusion conformation, for example by preventing the HRB helix from associating with the HRA helix, thus preventing refolding to the post-fusion conformation.

A stabilized pre-fusion RSV F protein of the invention may comprise a disulfide bond between any two structural elements, or between one structural element and the remainder of the RSV F protein, or between a structural element of one subunit of a trimer and a structural element of another subunit of the same trimer. Generally, a first amino acid in one structural element and a second amino acid that is in a different structural element, or the same structural element on a separate monomer, and that is also in close proximity to the first amino acid in the prefusion model are selected for replacement with cysteine. The distance between the residues (e.g., the alpha carbons) can be less than about 10 Å, less than about 8 Å, less than about 6 Å, less than about 5 Å, less than about 4 Å, or less than about 3 Å. The cysteine replacements of the first amino acid and the second amino acid, and a disulfide bond between them can be modeled. The length of the modeled disulfide bond, in some embodiments, does not exactly match the ˜2 Å length considered to be optimal for disulfide bonds. Preferably, the modeled disulfide bond length (distance between sulfur nuclei) is about 0.5 Å-3.5 Å, about 1.0 Å-3.0 Å, or about 1.5 Å-2.5 Å, which, due to structural flexibility, are expected to form disulfide bonds in the protein.

In one embodiment, pre-fusion RSV F protein may be stabilized in the pre-fusion conformation through the introduction of at least one cysteine mutation in a first structural element in close proximity to at least one other cysteine (natural or introduced) in a second structural element or the remaining RSV F head region. Disulfide bonds form between the introduced cysteine that prevent the post-fusion HRA-HRB six-helix bundle from forming. For example, pre-fusion RSV F protein may be stabilized in the pre-fusion conformation through the introduction of at least one cysteine mutation in the HRA helix region, HRB helix region, the fusion peptide, helix α1, helix α2, helix α3, helix α4, strand β1, strand β2, DI, DII, or DIII in close proximity to at least one other cysteine (natural or introduced) in a different structural region (e.g., selected from the HRA helix region, HRB helix region, the fusion peptide, helix α1, helix α2, helix α3, helix α4, strand β1, strand β2, DI, DII, or DIII), thereby forming one or more disulfide bridges that would prevent the post-fusion HRA-HRB six-helix bundle from forming.

The cysteines may be introduced to the HRB or the HRB linker to create disulfide bonds between the cysteines. In one embodiment, one or more cysteines may be introduced to the HRB linker and helix region (approximately residues 452 to 515) to form disulfides with portions of the RSV F head region. In another embodiment, a disulfide bond between the HRB linker or helix and the remainder of the RSV F protein may be used to stabilize the protein in the pre-fusion conformation. In a preferred embodiment, a disulfide bridge is formed between the HRB pre-fusion stalk and the DI or DII region at the “top” of the head (e.g., M396C+F483C).

In a preferred embodiment, the pre-fusion RSV F protein comprises two cysteine mutations, M396C and F483C, thereby comprising a disulfide bond between the HRB pre-fusion stalk and the DI region.

In other preferred embodiments, a disulfide bridge is formed between the HRA region and DIII region. For example, the RSV F protein can contain amino acid replacements selected from the group consisting of V56C+V164C, I57C+S190C, T58C+V164C, N165C+V296C, K168C+V296C, and combinations thereof.

In one embodiment, the pre-fusion RSV F protein comprises a first cysteine mutation in the HRA region, and a second cysteine (natural or introduced) in the fusion peptide, helix α1, helix α2, helix α3, helix α4, strand β1, strand β2 of the pre-fusion HRA region, or DIII. In this embodiment, the protein comprises a disulfide bond between the first and second cysteine that prevents the post fusion HRA-HRB six-helix bundle from forming.

In one embodiment, the pre-fusion RSV F protein comprises a first cysteine mutation in the HRB helix region, and a second cysteine (natural or introduced) in DI or DII. In this embodiment, the protein comprises a disulfide bond between the first and second cysteine that prevents the post-fusion HRA-HRB six-helix bundle from forming.

In one embodiment, the pre-fusion RSV F protein comprises a first cysteine mutation in the fusion peptide, and a second cysteine (natural or introduced) in the HRA region, helix α1, helix α2, helix α3, helix α4, strand β1, strand β2 or DIII. In this embodiment, the protein comprises a disulfide bond between the first and second cysteine that prevents the post-fusion HRA elongated helix from forming.

In one embodiment, the pre-fusion RSV F protein comprises a first cysteine mutation in the helix α1, and a second cysteine (natural or introduced) in the HRA region, the fusion peptide, helix α2, helix α3, helix α4, strand β1, strand β2, or DIII. In this embodiment, the protein comprises a disulfide bond between the first and second cysteine that prevents the post-fusion HRA-HRB six-helix bundle from forming.

In one embodiment, the pre-fusion RSV F protein comprises a first cysteine mutation in the helix α2, and a second cysteine (natural or introduced) in the HRA region, the fusion peptide, helix α1, helix α3, helix α4, strand β1, strand β2, or DIII. In this embodiment, the protein comprises a disulfide bond between the first and second cysteine that prevents the post-fusion HRA-HRB six-helix bundle from forming.

In one embodiment, the pre-fusion RSV F protein comprises a first cysteine mutation in the helix α3, and a second cysteine (natural or introduced) in the HRA region, the fusion peptide, helix α1, helix α2, helix α4, strand β1, strand β2, or DIII. In this embodiment, the protein comprises a disulfide bond between the first and second cysteine that prevents the post-fusion HRA-HRB six-helix bundle from forming.

In one embodiment, the pre-fusion RSV F protein comprises a first cysteine mutation in the helix α4, and a second cysteine (natural or introduced) in the HRA region, the fusion peptide, helix α1, helix α2, helix α3, strand β1, strand β2, or DIII. In this embodiment, the protein comprises a disulfide bond between the first and second cysteine that prevents the post-fusion HRA-HRB six-helix bundle from forming.

In one embodiment, the pre-fusion RSV F protein comprises a first cysteine mutation in the strand β1, and a second cysteine (natural or introduced) in the HRA region, the fusion peptide, helix α1, helix α2, helix α3, helix α4, strand β2, or DIII. In this embodiment, the protein comprises a disulfide bond between the first and second cysteine that prevents the post-fusion HRA-HRB six-helix bundle from forming.

In one embodiment, the pre-fusion RSV F protein comprises a first cysteine mutation in the strand β2, and a cysteine (natural or introduced) in the HRA helix region, the fusion peptide, helix α1, helix α2, helix α3, helix α4, strand β1, DI, DII, or DIII. In this embodiment, the protein comprises a disulfide bond between the first and second cysteine that prevents the post-fusion HRA-HRB six-helix bundle from forming.

In one embodiment, the pre-fusion RSV F protein comprises a first cysteine mutation in the DI region, and a second cysteine (natural or introduced) in the HRB helix region. In this embodiment, the protein comprises a disulfide bond between the first and second cysteine that prevents the post-fusion HRA-HRB six-helix bundle from forming.

In one embodiment, the pre-fusion RSV F protein comprises a first cysteine mutation in the DII region, and a second cysteine (natural or introduced) in the HRB helix region. In this embodiment, the protein comprises a disulfide bond between the first and second cysteine that prevents the post-fusion HRA-HRB six-helix bundle from forming.

In one embodiment, the pre-fusion RSV F protein comprises a first cysteine mutation in the DIII region, and a second cysteine (natural or introduced) in the HRA helix region. In this embodiment, the protein comprises a disulfide bond between the first and second cysteine that prevents the post-fusion HRA-HRB six-helix bundle from forming.

In another embodiment, the pre-fusion RSV F protein comprises a first introduced cysteine in the HRA helix region, HRB helix region, the fusion peptide, helix α1, helix α2, helix α3, helix α4, strand β1, strand β2, DI, DII, or DIII region and a second cysteine (natural or introduced) in any other region of the RSV F protein that is in close proximity to the introduced cysteine. In this embodiment, the protein comprises a disulfide bond between the first and second cysteine that prevents the post-fusion HRA-HRB six-helix bundle from forming.

In a specific embodiment, the pre-fusion RSV F protein comprises two cysteine mutations selected from the group consisting of V56C+V164C, I57C+S190C, T58C+V164C, N165C+V296C, and K168C+V296C, and combinations thereof, thereby comprising a disulfide bridge between the HRA region and the DIII region.

In one embodiment, the pre-fusion RSV F protein comprises two cysteine mutations, V56C and V164C, thereby comprising a disulfide bridge between the HRA region and the DIII region.

In one embodiment, the pre-fusion RSV F protein comprises two cysteine mutations, I57C and S190C, thereby comprising a disulfide bridge between the HRA region and the DIII region.

In one embodiment, the pre-fusion RSV F protein comprises two cysteine mutations, T58C and V164C, thereby comprising a disulfide bridge between the HRA region and the DIII region.

In one embodiment, the pre-fusion RSV F protein comprises two cysteine mutations, N165C and T296C, thereby comprising a disulfide bridge between the HRA region and the DIII region.

In one embodiment, the pre-fusion RSV F protein comprises two cysteine mutations, K168C and T296C, thereby comprising a disulfide bridge between the HRA region and the DIII region.

In one embodiment, the pre-fusion RSV F protein comprises two cysteine mutations, M396C+F483C, thereby comprising a disulfide bridge between the HRB region and the DII region.

The RSV F pre-fusion protein of the present invention is stabilized in the pre-fusion conformation by mutations that stabilize the pre-fusion subunit, which forms trimers.

In some embodiments, the RSV F pre-fusion protein of the present invention is a trimer of RSV F monomers, and pre-fusion conformation is stabilized by one or more disulfide bonds between cysteine residues that are introduced into different monomers.

Exemplary amino acid sequences of RSV F monomers that contain introduced cysteine residues that stabilize the pre-fusion conformation are presented below (SEQ ID NOS: 4-9). The presented sequences contain a signal peptide and a HIS tag (GGSAGSGHHHHHH; SEQ ID NO:3). The pre-fusion RSV F protein of the invention can contain any of the amino acid sequences shown below, with or without the signal peptide and/or HIS tag.

>RSV F HRA disulfide1 (V56C + V164C)
(SEQ ID NO: 4)
MELLILKANAITTILTAVTFCFASGQNITEEFYQS
TCSAVSKGYLSALRTGWYTSCITIELSNIKENKCN
GTDAKVKLIKQELDKYKNAVTELQLLMQSTPATNN
RARRELPRFMNYTLNNAKKTNVTLSKKRKRRFLGF
LLGVGSAIASGVAVSKVLHLEGECNKIKSALLSTN
KAVVSLSNGVSVLTSKVLDLKNYIDKQLLPIVNKQ
SCSISNIETVIEFQQKNNRLLEITREFSVNAGVTT
PVSTYMLTNSELLSLINDMPITNDQKKLMSNNVQI
VRQQSYSIMSIIKEEVLAYVVQLPLYGVIDTPCWK
LHTSPLCTTNTKEGSNICLTRTDRGWYCDNAGSVS
FFPQAETCKVQSNRVFCDTMNSLTLPSEVNLCNVD
IFNPKYDCKIMTSKTDVSSSVITSLGAIVSCYGKT
KCTASNKNRGIIKTFSNGCDYVSNKGVDTVSVGNT
LYYVNKQEGKSLYVKGEPIINFYDPLVFPSDEFDA
SISQVNEKINQSLAFIRKSDELLHNVNAGKSTTNG
GSAGSGHHHHHH
>RSV F HRA disulfide2 (I57C + S190C)
(SEQ ID NO: 5)
MELLILKANAITTILTAVTFCFASGQNITEEFYQS
TCSAVSKGYLSALRTGWYTSVCTIELSNIKENKCN
GTDAKVKLIKQELDKYKNAVTELQLLMQSTPATNN
RARRELPRFMNYTLNNAKKTNVTLSKKRKRRFLGF
LLGVGSAIASGVAVSKVLHLEGEVNKIKSALLSTN
KAVVSLSNGVSVLTCKVLDLKNYIDKQLLPIVNKQ
SCSISNIETVIEFQQKNNRLLEITREFSVNAGVTT
PVSTYMLTNSELLSLINDMPITNDQKKLMSNNVQI
VRQQSYSIMSIIKEEVLAYVVQLPLYGVIDTPCWK
LHTSPLCTTNTKEGSNICLTRTDRGWYCDNAGSVS
FFPQAETCKVQSNRVFCDTMNSLTLPSEVNLCNVD
IFNPKYDCKIMTSKTDVSSSVITSLGAIVSCYGKT
KCTASNKNRGIIKTFSNGCDYVSNKGVDTVSVGNT
LYYVNKQEGKSLYVKGEPIINFYDPLVFPSDEFDA
SISQVNEKINQSLAFIRKSDELLHNVNAGKSTTNG
GSAGSGHHHHHH
>RSV F HRA disulfide3 (T58C + V164C)
(SEQ ID NO: 6)
MELLILKANAITTILTAVTFCFASGQNITEEFYQS
TCSAVSKGYLSALRTGWYTSVICIELSNIKENKCN
GTDAKVKLIKQELDKYKNAVTELQLLMQSTPATNN
RARRELPRFMNYTLNNAKKTNVTLSKKRKRRFLGF
LLGVGSAIASGVAVSKVLHLEGECNKIKSALLSTN
KAVVSLSNGVSVLTSKVLDLKNYIDKQLLPIVNKQ
SCSISNIETVIEFQQKNNRLLEITREFSVNAGVTT
PVSTYMLTNSELLSLINDMPITNDQKKLMSNNVQI
VRQQSYSIMSIIKEEVLAYVVQLPLYGVIDTPCWK
LHTSPLCTTNTKEGSNICLTRTDRGWYCDNAGSVS
FFPQAETCKVQSNRVFCDTMNSLTLPSEVNLCNVD
IFNPKYDCKIMTSKTDVSSSVITSLGAIVSCYGKT
KCTASNKNRGIIKTFSNGCDYVSNKGVDTVSVGNT
LYYVNKQEGKSLYVKGEPIINFYDPLVFPSDEFDA
SISQVNEKINQSLAFIRKSDELLHNVNAGKSTTNG
GSAGSGHHHHHH
>RSV F HRA disulfide4 (N165C + V296C)
(SEQ ID NO: 7)
MELLILKANAITTILTAVTFCFASGQNITEEFYQS
TCSAVSKGYLSALRTGWYTSVITIELSNIKENKCN
GTDAKVKLIKQELDKYKNAVTELQLLMQSTPATNN
RARRELPRFMNYTLNNAKKTNVTLSKKRKRRFLGF
LLGVGSAIASGVAVSKVLHLEGEVCKIKSALLSTN
KAVVSLSNGVSVLTSKVLDLKNYIDKQLLPIVNKQ
SCSISNIETVIEFQQKNNRLLEITREFSVNAGVTT
PVSTYMLTNSELLSLINDMPITNDQKKLMSNNVQI
VRQQSYSIMSIIKEECLAYVVQLPLYGVIDTPCWK
LHTSPLCTTNTKEGSNICLTRTDRGWYCDNAGSVS
FFPQAETCKVQSNRVFCDTMNSLTLPSEVNLCNVD
IFNPKYDCKIMTSKTDVSSSVITSLGAIVSCYGKT
KCTASNKNRGIIKTFSNGCDYVSNKGVDTVSVGNT
LYYVNKQEGKSLYVKGEPIINFYDPLVFPSDEFDA
SISQVNEKINQSLAFIRKSDELLHNVNAGKSTTNG
GSAGSGHHHHHH
>RSV F HRA disulfide5 (K168C + V296C)
(SEQ ID NO: 8)
MELLILKANAITTILTAVTFCFASGQNITEEFYQS
TCSAVSKGYLSALRTGWYTSVITIELSNIKENKCN
GTDAKVKLIKQELDKYKNAVTELQLLMQSTPATNN
RARRELPRFMNYTLNNAKKTNVTLSKKRKRRFLGF
LLGVGSAIASGVAVSKVLHLEGEVNKICSALLSTN
KAVVSLSNGVSVLTSKVLDLKNYIDKQLLPIVNKQ
SCSISNIETVIEFQQKNNRLLEITREFSVNAGVTT
PVSTYMLTNSELLSLINDMPITNDQKKLMSNNVQI
VRQQSYSIMSIIKEECLAYVVQLPLYGVIDTPCWK
LHTSPLCTTNTKEGSNICLTRTDRGWYCDNAGSVS
FFPQAETCKVQSNRVFCDTMNSLTLPSEVNLCNVD
IFNPKYDCKIMTSKTDVSSSVITSLGAIVSCYGKT
KCTASNKNRGIIKTFSNGCDYVSNKGVDTVSVGNT
LYYVNKQEGKSLYVKGEPIINFYDPLVFPSDEFDA
SISQVNEKINQSLAFIRKSDELLHNVNAGKSTTNG
GSAGSGHHHHHH
>RSV F HRB disulfide (M396C + F483C)
(SEQ ID NO: 9)
MELLILKANAITTILTAVTFCFASGQNITEEFYQS
TCSAVSKGYLSALRTGWYTSVITIELSNIKENKCN
GTDAKVKLIKQELDKYKNAVTELQLLMQSTPATNN
RARRELPRFMNYTLNNAKKTNVTLSKKRKRRFLGF
LLGVGSAIASGVAVSKVLHLEGEVNKIKSALLSTN
KAVVSLSNGVSVLTSKVLDLKNYIDKQLLPIVNKQ
SCSISNIETVIEFQQKNNRLLEITREFSVNAGVTT
PVSTYMLTNSELLSLINDMPITNDQKKLMSNNVQI
VRQQSYSIMSIIKEEVLAYVVQLPLYGVIDTPCWK
LHTSPLCTTNTKEGSNICLTRTDRGWYCDNAGSVS
FFPQAETCKVQSNRVFCDTMNSLTLPSEVNLCNVD
IFNPKYDCKICTSKTDVSSSVITSLGAIVSCYGKT
KCTASNKNRGIIKTFSNGCDYVSNKGVDTVSVGNT
LYYVNKQEGKSLYVKGEPIINFYDPLVCPSDEFDA
SISQVNEKINQSLAFIRKSDELLHNVNAGKSTTNG
GSAGSGHHHHHH

### Destabilization of Post-Fusion RSV F

A major feature of the RSV F post-fusion structure is the 6-helix bundle in the stalk region. In the post-fusion conformation, the three HRA helices and the three HRB helices form a very stable 6-helix bundle. An alternative strategy for producing a stabilized pre-fusion RSV F protein of the invention, is by destabilizing the post-fusion 6-helix bundle, and/or preventing the 6-helix bundle formation (e.g., through deletion of the HRB helix, introduction of point mutations, addition of glycosylation or other modification sites).

The 6-helix bundle formation may be prevented by deleting the HRA helix or the HRB helix.

Preferably, the HRB helical region is deleted or mutated to prevent formation of the post-fusion conformation. The HRB region forms the stalk of the RSV F pre-fusion conformation, is in close proximity to the viral membrane and likely does not contain important neutralizing epitopes. Deletion of the HRB helix (residues 484 and C-terminal) may prevent the refolding of the RSV F protein from the pre-fusion state to the post-fusion state.

A stabilized pre-fusion RSV F protein of the invention may comprise the RSV ectodomain sequence with the HRB region deleted or mutated, and preferably further comprises an additional mutation or deletion to the remaining ectodomain sequence. For example, the RSV ectodomain may comprise one or more introduced cysteines to create disulfide bridges that stabilize the prefusion structure as described herein, mutated or deleted furin cleavage sites, mutated or deleted fusion peptide sequence, or other mutations previously described in WO 2011/008974, incorporated herein in its entirety. The RSV ectodomain may be the ectodomain of a naturally occurring RSV F protein, or it may contain mutations in addition to the deletions and/or mutations of the HRA or HRB region.

In one embodiment, the stabilized pre-fusion RSV F protein comprises an ectodomain of a naturally occurring RSV F protein in which the HRB region is deleted and one or more mutations that prevent cleavage at one or both of the furin cleavage sites (i.e, amino acids 109 and 136 of SEQ ID NOS:1 and 2) are present.

In one embodiment, the stabilized pre-fusion RSV F protein comprises an ectodomain of a naturally occurring RSV F protein in which the HRB region is deleted and the fusion peptide is mutated (amino acids 137 and 153 of SEQ ID NOS: 1 or 2). For example, this region can be deleted in whole or in part.

In another embodiment, the stabilized pre-fusion RSV F protein comprises a wild-type RSV ectodomain in which the HRB region and the fusion peptide is deleted, in whole or in part.

In another embodiment, the stabilized pre-fusion RSV F protein comprises a wild-type RSV ectodomain in which the HRB region is deleted and an oligomerization sequence has been added. When an oligomerization sequence is present, it is preferably a trimerization sequence. Suitable oligomerization sequences are well known in the art and include, for example, the coiled coil of the yeast GCN4 leucine zipper protein, trimerizing sequence from bacteriophage T4 fibritin (“foldon”), and the trimer domain of influenza HA.

In another embodiment, the stabilized pre-fusion RSV F protein comprises a wild-type RSV ectodomain in which the HRB region is deleted and the p27 region is mutated (amino acids 110-136 of SEQ ID NOS: 1 or 2), including deletion of the p27 region in whole or in part. For example, lysine and/or arginine residues in the p27 region (about amino acids 110-136 of SEQ ID NOS: 1 or 2) can be substituted or deleted.

In another embodiment, the stabilized pre-fusion RSV F protein comprises a wild-type RSV ectodomain in which the HRB region is deleted and an amino acid sequence that provides a protease cleavage site is added. Generally, the amino acid sequence that provides a protease cleavage site will be located within about 60 amino acids, about 50 amino acids, about 40 amino acids, about 30 amino acids, about 20 amino acids, about 10 amino acids, or substantially adjacent to the amino terminus of the transmembrane domain (amino acid 525 of SEQ ID NO:1 or 2).

An exemplary amino acid sequence of an RSV F monomer in which the fusion peptide and HRB are deleted to stabilize the pre-fusion conformation is presented below (SEQ ID NO: 10). The presented sequence contains a signal peptide and a HIS tag (GGSAGSGHHHHHH; SEQ ID NO:3). The pre-fusion RSV F protein of the invention can contain the amino acid sequences shown below, with or without the signal peptide and/or HIS tag.

>RSV F delHRB fusion peptide deletion HIS
(SEQ ID NO: 10)
MELLILKANAITTILTAVTFCFASGQNITEEFYQS
TCSAVSKGYLSALRTGWYTSVITIELSNIKENKCN
GTDAKVKLIKQELDKYKNAVTELQLLMQSTPATNN
RARRELPRFMNYTLNNAKKTNVTLSKKRKRRSAIA
SGVAVSKVLHLEGEVNKIKSALLSTNKAVVSLSNG
VSVLTSKVLDLKNYIDKQLLPIVNKQSCSISNIET
VIEFQQKNNRLLEITREFSVNAGVTTPVSTYMLTN
SELLSLINDMPITNDQKKLMSNNVQIVRQQSYSIM
SIIKEEVLAYVVQLPLYGVIDTPCWKLHTSPLCTT
NTKEGSNICLTRTDRGWYCDNAGSVSFFPQAETCK
VQSNRVFCDTMNSLTLPSEVNLCNVDIFNPKYDCK
IMTSKTDVSSSVITSLGAIVSCYGKTKCTASNKNR
GIIKTFSNGCDYVSNKGVDTVSVGNTLYYVNKQEG
KSLYVKGEPIINFYDPLVFPSGGSAGSGHHHHHH

The stabilized pre-fusion RSV F protein of the invention may also be formed by hindering 6-helix bundle formation in the HRA or HRB regions (approximately residues 154-212 and 484 to 513, respectively) through engineered point mutations or introduction of glycosylation sites (e.g., AsnXaaSer/Thr; SEQ ID NO:11) or other modification sites (e.g., lipidation, phosphorylation). Glycosylation sites, or other post-translational modification sites or point mutations which interfere with 6-helix bundle formation through electrostatic or steric hindrance, can be introduced through the HRA or HRB helical regions. Glycosylation can interfere with 6-helix bundle formation and prevent flipping of the RSV F construct to the post-fusion conformation.

A stabilized pre-fusion RSV F protein of the invention may comprise one or more mutations that form one or more glycosylation sites in the HRA or HRB helical regions. The RSV F protein may contain mutations or deletions in addition to those that introduce the glycosylation sites in the HRA or HRB helical regions.

In one embodiment, the RSV F protein comprises S173N and N175T mutations and the pre-fusion RSV F protein has a glycosylation site on current serine residue 173.

In another embodiment, the RSV F protein comprises a mutation A177T, so that the pre-fusion RSV F protein has a glycosylation site on current residue N175.

In another embodiment, the RSV F protein comprises G184N and S186T mutations and the pre-fusion RSV F protein has a glycosylation site on current residue G184.

Chimeric Pre-Fusion F Structures with Relevant RSV F Epitopes

The invention also relates to chimeric pre-fusion F proteins that contain neutralizing epitopes from RSV F protein. Generally, the chimeric pre-fusion F proteins comprise a stabilized F protein from a different virus, such as the Parainfluenza Virus (PIV1, PIV2, PIV3, PIV4, PIV5), Newcastle Disease Virus (NDV), Sendai virus (SeV), Hendra Virus, Nipah Virus (NiV), human metapneumovirus or avian metapneumovirus, in which portions that are exposed on the surface of the protein are replaced with corresponding portions of RSV F. Preferably, the portions contain neutralizing epitopes of RSV F.

For example, any non-RSV (e.g., parainfluenza virus or metapneumovirus) F protein that is stabilized in the pre-fusion conformation (e.g., by virtue of a GCN trimer domain fused C-terminally to the HRB region), may be used as a template for the protein (i.e., an uncleaved NDV F-GCN fusion protein). For example, the SV5 of PIV5 pre-fusion F protein, described by Yin et al., 2006 (145) or the NDV pre-fusion F described by Swanson et al, 2010 (146) may be used in the chimeric F protein construct. The template may then be mutated to introduce known or suspected neutralizing epitopes of RSV F. Thus, the protein may have a pre-fusion F structure exhibiting the neutralizing epitopes, but not non-neutralizing epitopes, of RSV F. A clear benefit to this construct is that it would not raise non-neutralizing RSV F antibodies.

A pre-fusion chimera protein of the invention may comprise a parainfluenza virus F protein, stabilized in the pre-fusion conformation that is mutated to introduce neutralizing epitopes of RSV F. The parainfluenza virus F protein may be any parainfluenza virus F protein, preferably the SV5 of PIV5 pre-fusion F protein, described by Yin et al., 2006 (147) or the NDV pre-fusion F described by Swanson et al, 2010 (146). Exemplary neutralizing epitopes that may be introduced by mutation include the epitopes disclosed in Table 1. For example, the amino acids that form the epitopes recognized by the antibodies listed in Table 1 can be introduced into the corresponding positions (e.g., identified by structural comparison, such as structure based alignment) of a non RSV-F protein (e.g., parainfluenza virus F protein or metapneumovirus F protein) that is stabilized in the pre-fusion conformation. For example, the chimeric F protein can contain the RSV F site A epitope or site C epitope. In particular example, the chimeric F protein contains one or more RSV F residues selected from the amino acid residues at positions 262-276 of RSV F. In another particular example, the chimeric F protein contains one or more RSV F residues selected from the amino acid residues at positions 429-447 of RSV F.

TABLE 1
Neutralizing epitopes of RSV F^{a}

| Site^{b} | mAb | Residues | Method | Reference |
|---|---|---|---|---|
| A | 11 | N268I | Escape^{c} | (149) |
| A | 151 | K272N | Escape | (150) |
| A | 1129 | S275F | Escape | (150) |
| A | 1153 | N262S | Escape | (150) |
| A | 1200 | K272N | Escape | (150) |
| A | 1214 | N276Y | Escape | (150) |
| A | 1237 | N276Y | Escape | (150) |
| A | 47F | N262Y, N268I | Escape | (151) |
| A | 7C2 | K272E, K272T | Escape | (149) |
| A | B4 | K272T | Escape | (149) |
| A | Fab 19^{d} | I266M | Escape | (149) |
| A | AK13A2 | N262Y | Escape | (149) |
| A | PVZ^{e} | K272M, K272Q, N268I | Escape | (152) |
| A | PVZ^{e} | K272M, K272T, S275F | Engineered^{f} | (153) |
| A | MVZ^{g} | N262, N268, D269, K272, | Structure^{h} | (141) |
| | | S275 | | |
| C | 7.936 | I432T, K433T, V447A | Escape | (154) |
| C | 9.432 | S436F | Escape | (154) |
| C | 19^{d} | R429S | Escape | (149) |
| C | 19^{d} | R429K, R429S, G430A | Engineered | (153) |
| C | 20 | R429S | Escape | (149) |
| C | 101F | K433T | Escape | (155) |
| C | 101F | K433D, K433L, K433N, | Engineered | (153) |
| | | K433Q, K433R | | |
| C | 101F | R429, I431, I432, K433, | Structure | (148) |
| | | T434, F435, S436, N437 | | |

^{a}Results of studies using peptide binding or peptide inhibition are not included in this table.
^{b}Sites are based on the competition and cross-neutralization analysis of Beeler et al., 1989 (156).
^{c}An escape mutation is included if it is the sole mutation in an antibody-resistant strain.
^{d} Fab 19 and 19 are unrelated antibodies. The similar names are coincidental.
^{e}Palivizumab
^{f}Engineered mutations in intact recombinant RSV F that allowed intact processing, full fusion activity and reduced monoclonal antibody binding to less than 15% of wild type are included.
^{g}Motavizumab
^{h}Residues from peptides in peptide-Fab complex structures are included if either their side chain or backbone atoms make significant contact with the antibody. The biological significance of the peptide-antibody interactions observed in these structural studies has been confirmed by other techniques.

In one embodiment, the chimeric F protein of the invention comprises the HRA region of RSV F (residues 137-212) in place of the equivalent HRA residues of NDV-GCN pre-fusion F. Thus, prefusion F protein (NDV-GCN) is expressed with potential neutralizing epitope sites (HRA region of RSV F) on the top of the pre-fusion F head, permitting neutralizing RSV F antibodies to be elicited. Additional neutralizing epitopes may be added if desired, for example the motavizumab epitope or the 101F epitope.

An exemplary amino acid sequence of a chimeric protein containing prefusion NDV F mutated to include RSV F HRA amino acid sequences is presented below (SEQ ID NO: 13). The presented sequences contain a signal peptide, a GCN-trimerization domain and a HIS tag. The chimeric pre-fusion F protein of the invention can contain the amino acid sequence shown below, with or without the signal peptide and/or GCN-trimerization domain and/or HIS tag.

NDV RSV HRA prefusion (underlined portion is RSV HRA, italicized portion is C-terminal GCN trimeric domain and HIS6 (SEQ ID NO: 12) affinity purification tag) (SEQ ID NO: 13).

MGSRSSTRIPVPLMLTVRVMLALSCVCPTSALDGRP
LAAAGIVVTGDKAVNIYTSSQTGSIIIKLLPNMPK
DKEACAKAPLEAYNRTLTTLLTPLGDSIRRIQESV
TTSGGGKQGRLIGAIIGFLGFLLGVGSAIASGVAV
SKVLHLEGEVNKIKSALLSTNKAVVSLSNGVSVLT
SKVLDLKNYIDKQLLPIVNKQSCIKITQQVGVELN
LYLTELTTVFGPQITSPALTQLTIQALYNLAGGNM
DYLLTKLGVGNNQLSSLISSGLITGNPILYDSQTQ
LLGIQVTLPSVGNLNNMRATYLETLSVSTTKGFAS
ALVPKVVTQVGSVIEELDTSYCIETDLDLYCTRIV
TFPMSPGIYSCLSGNTSACMYSKTEGALTTPYMTL
KGSVIANCKMTTCRCADPPGIISQNYGEAVSLIDR
QSCNILSLDGITLRLSGEFDATYQKNISIQDSQVI
VTGNLDISTELGNVNNSISNALDKLEESNSKLDKV
EDKIEEILSKIYHIENEIARIKKLIGEAGGPLVPR
GSHHHHHH

### The RSV F Glycoprotein

The F glycoprotein of RSV directs viral penetration by fusion between the virion envelope and the host cell plasma membrane. It is a type I single-pass integral membrane protein having four general domains: N-terminal ER-translocating signal sequence (SS), ectodomain (ED), transmembrane domain (TM), and a cytoplasmic tail (CT). CT contains a single palmitoylated cysteine residue. The sequence of F protein is highly conserved among RSV isolates, but is constantly evolving (7). Unlike most paramyxoviruses, the F protein in RSV can mediate entry and syncytium formation independent of the other viral proteins (HN is usually necessary in addition to F in other paramyxoviruses).

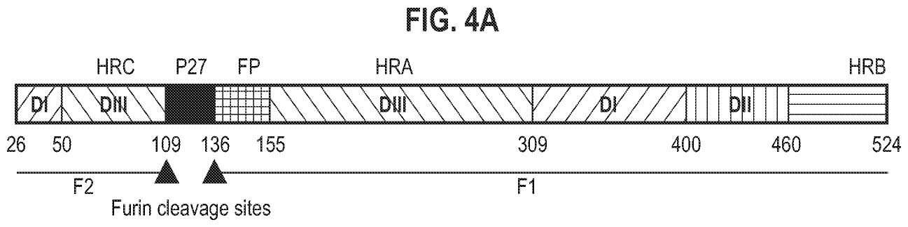

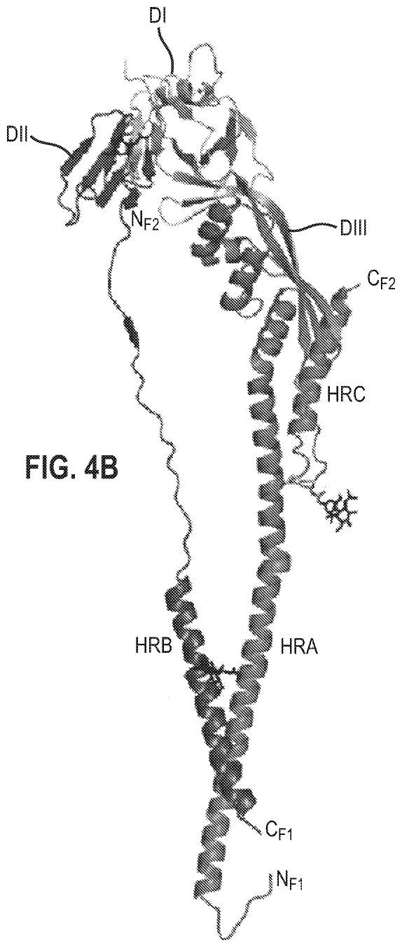

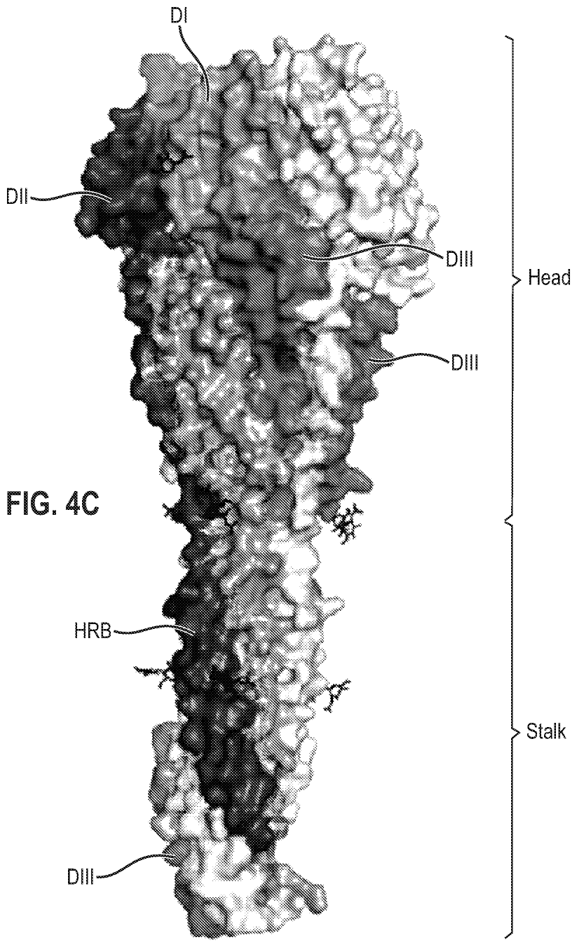

The hRSV F mRNA is translated into a 574 amino acid precursor protein designated F₀, which contains a signal peptide sequence at the N-terminus that is removed by a signal peptidase in the endoplasmic reticulum. F₀ is cleaved at two sites (a.a. 109/110 and 136/137) by cellular proteases (in particular furin) in the trans-Golgi, removing a short glycosylated intervening sequence and generating two subunits designated F₁ (˜50 kDa; C-terminus; residues 137-574) and F₂ (˜20 kDa; N-terminus; residues 1-109) (See, e.g., FIG. 4A). F₁ contains a hydrophobic fusion peptide at its N-terminus and also two hydrophobic heptad-repeat regions (HRA and HRB). HRA is near the fusion peptide and HRB is near to the transmembrane domain (See, e.g., FIG. 4A). The F₁-F₂ heterodimers are assembled as homotrimers in the virion.

RSV exists as a single serotype but has two antigenic subgroups: A and B. The F glycoproteins of the two groups are about 90% identical. The A subgroup, the B subgroup, or a combination or hybrid of both can be used in the invention. An example sequence for the A subgroup is SEQ ID NO: 1 (A2 strain; GenBank GI: 138251; Swiss Prot P03420), and for the B subgroup is SEQ ID NO: 2 (18537 strain; GI: 138250; Swiss Prot P13843). SEQ ID NO:1 and SEQ ID NO:2 are both 574 amino acid sequences. The signal peptide in A2 strain is a.a. 1-21, but in 18537 strain it is 1-22. In both sequences the TM domain is from about a.a. 530-550, but has alternatively been reported as 525-548.

```
SEQ ID NO: 1
    1 MELLILKANAITTILTAVTFCFASGQNITEEFYQSTCSAVSKGYLSALRTGWYTSVITIE 60
  61 LSNIKENKCNGTDAKVKLIKQELDKYKNAVTELQLLMQSTPPTNNRARRELPRFMNYTLN 120
121 NAKKTNVTLSKKRKRRFLGFLLGVGSAIASGVAVSKVLHLEGEVNKIKSALLSTNKAVVS 180
181 LSNGVSVLTSKVLDLKNYIDKQLLPIVNKQSCSISNIETVIEFQQKNNRLLEITREFSVN 240
241 AGVTTPVSTYMLTNSELLSLINDMPITNDQKKLMSNNVQIVRQQSYSIMSIIKEEVLAYV 300
301 VQLPLYGVIDTPCWKLHTSPLCTTNTKEGSNICLTRTDRGWYCDNAGSVSFFPQAETCKV 360
361 QSNRVFCDTMNSLTLPSEINLCNVDIFNPKYDCKIMTSKTDVSSSVITSLGAIVSCYGKT 420
421 KCTASNKNRGIIKTFSNGCDYVSNKGMDTVSVGNTLYYVNKQEGKSLYVKGEPIINFYDP 480
481 LVFPSDEFDASISQVNEKINQSLAFIRKSDELLHNVNAGKSTTNIMITTIIIVIIVILLS 540
541 LIAVGLLLYCKARSTPVTLSKDQLSGINNIAFSN 574
SEQ ID NO: 2
    1 MELLIHRSSAIFLTLAVNALYLTSSQNITEEFYQSTCSAVSRGYFSALRTGWYTSVITIE 60
  61 LSNIKETKCNGTDTKVKLIKQELDKYKNAVTELQLLMQNTPAANNRARREAPQYMNYTIN 120
121 TTKNLNVSISKKRKRRFLGFLLGVGSAIASGIAVSKVLHLEGEVNKIKNALLSTNKAVVS 180
181 LSNGVSVLTSKVLDLKNYINNRLLPIVNQQSCRISNIETVIEFQQMNSRLLEITREFSVN 240
241 AGVTTPLSTYMLTNSELLSLINDMPITNDQKKLMSSNVQIVRQQSYSIMSIIKEEVLAYV 300
301 VQLPIYGVIDTPCWKLHTSPLCTTNIKEGSNICLTRTDRGWYCDNAGSVSFFPQADTCKV 360
361 QSNRVFCDTMNSLTLPSEVSLCNTDIFNSKYDCKIMTSKTDISSSVITSLGAIVSCYGKT 420
421 KCTASNKNRGIIKTFSNGCDYVSNKGVDTVSVGNTLYYVNKLEGKNLYVKGEPIINYYDP 480
481 LVFPSDEFDASISQVNEKINQSLAFIRRSDELLHNVNTGKSTTNIMITTIIIVIIVVLLS 540
541 LIAIGLLLYCKAKNTPVTLSKDQLSGINNIAFSK 574
```

The invention may use any desired RSV F amino acid sequence, such as the amino acid sequence of SEQ ID NO: 1 or 2, or a sequence having identity to SEQ ID NO: 1 or 2. Typically it will have at least 75% identity to SEQ ID NO: 1 or 2 e.g., at least 80%, at least 85%, at least 90%, at least 95%, at least 97%, at least 98%, at least 99%, identity to SEQ ID NO:1 or 2. The sequence may be found naturally in RSV.

Where the invention uses an ectodomain of F protein, in whole or in part, it may comprise:
(i) a polypeptide comprising about amino acid 22-525 of SEQ ID NO: 1.
(ii) a polypeptide comprising about amino acids 23-525 of SEQ ID NO: 2.
(iii) a polypeptide comprising an amino acid sequence having at least 75% identity (e.g., at least 80%, at least 85%, at least 90%, at least 95%, at least 97%, at least 98%, at least 99% identity) to (i) or (ii).
(iv) a polypeptide comprising a fragment of (i), (ii) or (iii), wherein the fragment comprises at least one F protein epitope. The fragment will usually be at least about 100 amino acids long, e.g., at least about 150, at least about 200, at least about 250, at least about 300, at least about 350, at least about 400, at least about 450 amino acids long.

The ectodomain can be an F₀ form with or without the signal peptide, or can comprise two separate peptide chains (e.g., an F₁ subunit and a F₂ subunit) that are associated with each other, for example, the subunits may be linked by a disulfide bridge. Accordingly, all or a portion of about amino acid 101 to about 161, such as amino acids 110-136, may be absent from the ectodomain. Thus the ectodomain, in whole or in part, can comprise:
(v) a first peptide chain and a second peptide chain that is associated with the first polypeptide chain, where the first peptide chain comprises an amino acid sequence having at least 75% identity (e.g., at least 80%, at least 85%, at least 90%, at least 95%, at least 97%, at least 98%, at least 99%, or even 100% identity) to about amino acid 22 to about amino acid 101 of SEQ ID NO: 1 or to about amino acid 23 to about amino acid 101 of SEQ ID NO: 2, and the second peptide chain comprises an amino acid sequence having at least 75% identity (e.g., at least 80%, at least 85%, at least 90%, at least 95%, at least 97%, at least 98%, at least 99%, or even 100% identity) to about amino acid 162 to about 525 of SEQ ID NO: 1 or to about amino acid 162 to 525 of SEQ ID NO: 2.
(vi) a first peptide chain and a second peptide chain that is associated with the first polypeptide chain, where the first peptide chain comprises an amino acid sequence comprising a fragment of about amino acid 22 to about amino acid 101 of SEQ ID NO: 1 or of about amino acid 23 to about amino acid 109 of SEQ ID NO: 2, and the second peptide chain comprises a fragment of about amino acid 162 to about amino acid 525 of SEQ ID NO: 1 or of about amino acid 161 to about amino acid 525 of SEQ ID NO: 2. One or both of the fragments will comprise at least one F protein epitope. The fragment in the first peptide chain will usually be at least 20 amino acids long, e.g., at least 30, at least 40, at least 50, at least 60, at least 70, at least 80 amino acids long. The fragment in the second peptide chain will usually be at least 100 amino acids long, e.g., at least 150, at least 200, at least 250, at least 300, at least 350, at least 400, at least 450 amino acids long.
(vii) a molecule obtainable by furin digestion of (i), (ii), (iii) or (iv).

Thus an amino acid sequence used with the invention may be found naturally within RSV F protein (e.g., a soluble RSV F protein lacking TM and CT, about amino acids 522-574 of SEQ ID NOS: 1 or 2), and/or it may have one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30) single amino acid mutations (insertions, deletions or substitutions) relative to a natural RSV sequence. For instance, it is known to mutate F proteins to eliminate their furin cleavage sequences, thereby preventing intracellular processing. In certain embodiments, the RSV F protein lacks TM and CT (about amino acids 522-574 of SEQ ID NOS: 1 or 2) and contains one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30) single amino acid mutations (insertions, deletions or substitutions) relative to a natural RSV sequence.

### Furin-Cleavage, Trypsin-Cleavage and Fusion Peptide Mutations

If desired, post-fusion RSV F polypeptides or proteins may contain one or more mutations, for example, mutations that prevent cleavage at one or both of the furin cleavage sites (i.e., amino acids 109 and 136 of SEQ ID NOS: 1 and 2), that prevent cleavage of or introduce trypsin cleavage sites, mutations in the p27 region, and/or mutation in the fusion peptide. Such mutations can prevent aggregation of the soluble polypeptides or proteins and thereby facilitate purifications, can prevent cell-cell fusion if the RSV F protein is expressed on the surface of a cell, such as by expression from a viral replicon (e.g., alphavirus replicon particles), or if the RSV F protein is a component of a virus-like particle.

Examples of suitable furin cleavage mutations include replacement of amino acid residues 106-109 of SEQ ID NO: 1 or 2 with RARK (SEQ ID NO:14), RARQ (SEQ ID NO:15), QAQN (SEQ ID NO:16), or IEGR (SEQ ID NO:17). Alternatively, or in addition, amino acid residues 133-136 of SEQ ID NO: 1 or 2 can be replaced with RKKK (SEQ ID NO:18), AAAR, QNQN (SEQ ID NO:19), QQQR (SEQ ID NO:20) or IEGR (SEQ ID NO:17). (A indicates that the amino acid residue has been deleted.) These furin cleavage mutations can be combined, if desired, with other mutations described herein, such as trypsin cleavage mutations and fusion peptide mutations.

Examples of suitable trypsin cleavage mutations include deletion of any lysine or arginine residue between about position 101 and position 161 of SEQ ID NO:1 or 2, or replacement of any such lysine or arginine residue with an amino acid other than lysine or arginine. For example, lysine and/or arginine residues in the p27 region (about amino acids 110-136 of SEQ ID NOS: 1 or 2) can be substituted or deleted, including deletion of the p27 region in whole or in part.

The mutations described herein can be combined, if desired, in any combination. For example, furin mutations can be combined with partial or complete deletion of the fusion peptide region and/or deletion of the HRA or HRB helical region.

In addition to the mutations described above, e.g., furin-cleavage and fusion peptide mutations, or alternatively, soluble RSV F polypeptides or proteins, such as those that lack the transmembrane region and cytoplasmic tail, or HRA and HRB deletions, or cysteine mutations, may contain one or more oligomerization sequences. When an oligomerization sequence is present, it is preferably a trimerization sequence. Suitable oligomerization sequences are well known in the art and include, for example, the coiled coil of the yeast GCN4 leucine zipper protein, trimerizing sequence from bacteriophage T4 fibritin (“foldon”), and the trimer domain of influenza HA. These and other suitable oligomerization sequences are described in greater detail herein.

In particular embodiments, the sequence of the carboxy terminus of the RSV F polypeptide or protein, starting from position 480, is

(GCN)
(SEQ ID NO: 21)
PLVFPSDEFDASISQVNEKINQSLAFIRKSDE
LLHNVNDKIEEILSKIYHIENEIARIKKLIGE
(HA)
(SEQ ID NO: 22)
PLVFPSDEFDASISQVNEKINQSLAFIRKSDE
LLHNVNEKFHQIEKEFSEVEGRIQDLEK
(Idealized helix)
(SEQ ID NO: 23)
PLVFPSDEFDASISQINEKINQILAFIRKIDE
LLHNIN
(foldon short)
(SEQ ID NO: 24)
PLVFPSDEFDASISQVNEKINQSLAFIRKSDE
LLHNVNGSGYIPEAPRDGQAYVRKDGEWVLLS
TFL;
or
(foldon long)
(SEQ ID NO: 25)
PLVFPSDEFDASISQVNEKINQSLAFIRKSDE
LLHNVNNKNDDKGSGYIPEAPRDGQAYVRKDG
EWVLLSTFL

If desired, RSV F polypeptides or proteins that contain a transmembrane region may contain an added amino acid sequence that provides a protease cleavage site. This type of RSV F polypeptide or protein can be produced by expression on the surface of a cell, and recovered in soluble form after cleavage from the cell surface using an appropriate protease. Generally, the amino acid sequence that provides a protease cleavage site will be located within about 60 amino acids, about 50 amino acids, about 40 amino acids, about 30 amino acids, about 20 amino acids, about 10 amino acids, or substantially adjacent to the amino terminus of the transmembrane domain (amino acid 525 of SEQ ID NO:1 or 2). Many suitable amino acid sequences that are cleaved by commercially available proteases are well-known in the art. For example, thrombin cleaves the sequence LVPR (SEQ ID NO:26), factor Xa cleaves the sequence IEGR (SEQ ID NO:17) and enterokinase cleaves the sequence DDDDK (SEQ ID NO:27). These amino acid sequences can be introduced into an RSV F polypeptide.

Immunogenic polypeptides used according to the invention will usually be isolated or purified. Thus, they will not be associated with molecules with which they are normally, if applicable, found in nature. For example, an F protein used with the invention will not be in the form of a RSV virion (although it may be in the form of an artificial virion, such as a virosome or VLP).

Polypeptides will usually be prepared by expression in a recombinant host system. Generally, they (e.g., RSV ecto-domains) are produced by expression of recombinant constructs that encode the ecto-domains in suitable recombinant host cells, although any suitable methods can be used. Suitable recombinant host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster), avian cells (e.g., chicken, duck, and geese), bacteria (e.g., E. coli, Bacillus subtilis, and Streptococcus spp.), yeast cells (e.g., Saccharomyces cerevisiae, Candida albicans, Candida maltosa, Hansenual polymorpha, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophila) or combinations thereof. Many suitable insect cells and mammalian cells are well-known in the art. Suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Suitable mammalian cells include, for example, Chinese hamster ovary (CHO) cells, human embryonic kidney cells (HEK293 cells, typically transformed by sheared adenovirus type 5 DNA), NIH-3T3 cells, 293-T cells, Vero cells, HeLa cells, PERC.6 cells (ECACC deposit number 96022940), Hep G2 cells, MRC-5 (ATCC CCL-171), WI-38 (ATCC CCL-75), fetal rhesus lung cells (ATCC CL-160), Madin-Darby bovine kidney (“MDBK”) cells, Madin-Darby canine kidney (“MDCK”) cells (e.g., MDCK (NBL2), ATCC CCL34; or MDCK 33016, DSM ACC 2219), baby hamster kidney (BHK) cells, such as BHK21-F, HKCC cells, and the like. Suitable avian cells include, for example, chicken embryonic stem cells (e.g., EBx® cells), chicken embryonic fibroblasts, chicken embryonic germ cells, duck cells (e.g., AGE1.CR and AGE1.CR.pIX cell lines (ProBioGen) which are described, for example, in Vaccine 27:4975-4982 (2009) and WO2005/042728), EB66 cells, and the like.

Suitable insect cell expression systems, such as baculovirus systems, are known to those of skill in the art and described in, e.g., Summers and Smith, Texas Agricultural Experiment Station Bulletin No. 1555 (1987). Materials and methods for baculovirus/insert cell expression systems are commercially available in kit form from, inter alia, Invitrogen, San Diego CA. Avian cell expression systems are also known to those of skill in the art and described in, e.g., U.S. Pat. Nos. 5,340,740; 5,656,479; 5,830,510; 6,114,168; and 6,500,668; European Patent No. EP 0787180B; European Patent Application No. EP03291813.8; WO 03/043415; and WO 03/076601. Similarly, bacterial and mammalian cell expression systems are also known in the art and described in, e.g., Yeast Genetic Engineering (Barr et al., eds., 1989) Butterworths, London.

Recombinant constructs encoding pre-fusion RSV F protein can be prepared in suitable vectors using conventional methods. A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable baculovirus expression vector, such as pFastBac (Invitrogen), is used to produce recombinant baculovirus particles. The baculovirus particles are amplified and used to infect insect cells to express recombinant protein. For expression in mammalian cells, a vector that will drive expression of the construct in the desired mammalian host cell (e.g., Chinese hamster ovary cells) is used.

Pre-fusion RSV F protein polypeptides can be purified using any suitable methods. For example, methods for purifying pre-fusion RSV F polypeptides by immunoaffinity chromatography are known in the art. Ruiz-Arguello et al., J. Gen. Virol., 85:3677-3687 (2004). Suitable methods for purifying desired proteins including precipitation and various types of chromatography, such as hydrophobic interaction, ion exchange, affinity, chelating and size exclusion are well-known in the art. Suitable purification schemes can be created using two or more of these or other suitable methods. If desired, the pre-fusion RSV F protein polypeptides can include a “tag” that facilitates purification, such as an epitope tag or a HIS tag. Such tagged polypeptides can conveniently be purified, for example from conditioned media, by chelating chromatography or affinity chromatography.

The pre-fusion RSV F polypeptides may also be produced in situ by expression of nucleic acids that encode them in the cells of a subject. For example, by expression of a self-replicating RNA described herein.

Polypeptides may include additional sequences in addition to the pre-fusion RSV sequences. For example, a polypeptide may include a sequence to facilitate purification (e.g., a poly-His sequence). Similarly, for expression purposes, the natural leader peptide of F protein may be substituted for a different one. For example, reference 6 used a honeybee melittin leader peptide in place of the natural one.

### Self-Replicating RNA

The pre-fusion RSV-F polypeptides described herein can be produced by expression of recombinant nucleic acids that encode the polypeptides in the cells of a subject. Preferred nucleic acids that can be administered to a subject to cause the production of pre-fusion RSV-F polypeptides are self-replicating RNA molecules. The self-replicating RNA molecules of the invention are based on the genomic RNA of RNA viruses, but lack the genes encoding one or more structural proteins. The self-replicating RNA molecules are capable of being translated to produce non-structural proteins of the RNA virus and heterologous proteins encoded by the self-replicating RNA.

The self-replicating RNA generally contains at least one or more genes selected from the group consisting of viral replicase, viral proteases, viral helicases and other nonstructural viral proteins, and also comprise 5′- and 3′-end cis-active replication sequences, and if desired, a heterologous sequences that encode a desired amino acid sequences (e.g., a protein, an antigen). A subgenomic promoter that directs expression of the heterologous sequence can be included in the self-replicating RNA. If desired, the heterologous sequence may be fused in frame to other coding regions in the self-replicating RNA and/or may be under the control of an internal ribosome entry site (IRES).

Self-replicating RNA molecules of the invention can be designed so that the self-replicating RNA molecule cannot induce production of infectious viral particles. This can be achieved, for example, by omitting one or more viral genes encoding structural proteins that are necessary for the production of viral particles in the self-replicating RNA. For example, when the self-replicating RNA molecule is based on an alpha virus, such as Sinebis virus (SIN), Semliki forest virus and Venezuelan equine encephalitis virus (VEE), one or more genes encoding viral structural proteins, such as capsid and/or envelope glycoproteins, can be omitted. If desired, self-replicating RNA molecules of the invention can be designed to induce production of infectious viral particles that are attenuated or virulent, or to produce viral particles that are capable of a single round of subsequent infection.

A self-replicating RNA molecule can, when delivered to a vertebrate cell even without any proteins, lead to the production of multiple daughter RNAs by transcription from itself (or from an antisense copy of itself). The self-replicating RNA can be directly translated after delivery to a cell, and this translation provides a RNA-dependent RNA polymerase which then produces transcripts from the delivered RNA. Thus the delivered RNA leads to the production of multiple daughter RNAs. These transcripts are antisense relative to the delivered RNA and may be translated themselves to provide in situ expression of a gene product, or may be transcribed to provide further transcripts with the same sense as the delivered RNA which are translated to provide in situ expression of the encoded RSV-F polypeptide.

One suitable system for achieving self-replication is to use an alphavirus-based RNA replicon. These + stranded replicons are translated after delivery to a cell to give of a replicase (or replicase-transcriptase). The replicase is translated as a polyprotein which auto cleaves to provide a replication complex which creates genomic − strand copies of the + strand delivered RNA. These − strand transcripts can themselves be transcribed to give further copies of the + stranded parent RNA and also to give a subgenomic transcript which encodes the RSV-F polypeptide. Translation of the subgenomic transcript thus leads to in situ expression of the RSV-F polypeptide by the infected cell. Suitable alphavirus replicons can use a replicase from a sindbis virus, a semliki forest virus, an eastern equine encephalitis virus, a venezuelan equine encephalitis virus, etc.

A preferred self-replicating RNA molecule thus encodes (i) a RNA-dependent RNA polymerase which can transcribe RNA from the self-replicating RNA molecule and (ii) an RSV-F polypeptide. The polymerase can be an alphavirus replicase e.g. comprising alphavirus protein nsP4.

Whereas natural alphavirus genomes encode structural virion proteins in addition to the non structural replicase polyprotein, it is preferred that an alphavirus based self-replicating RNA molecule of the invention does not encode alphavirus structural proteins. Thus the self replicating RNA can lead to the production of genomic RNA copies of itself in a cell, but not to the production of RNA-containing alphavirus virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self replicating RNAs of the invention and their place is taken by gene(s) encoding the desired gene product, such that the subgenomic transcript encodes the desired gene product rather than the structural alphavirus virion proteins.

Thus a self-replicating RNA molecule useful with the invention may have two open reading frames. The first (5′) open reading frame encodes a replicase; the second (3′) open reading frame encodes an RSV-F polypeptide. In some embodiments the RNA may have additional (downstream) open reading frames e.g. that encode further desired gene products. A self-replicating RNA molecule can have a 5′ sequence which is compatible with the encoded replicase.

In one aspect, the self-replicating RNA molecule is derived from or based on an alphavirus. In other aspects, the self-replicating RNA molecule is derived from or based on a virus other than an alphavirus, preferably, a positive-stranded RNA viruses, and more preferably a picornavirus, flavivirus, rubivirus, pestivirus, hepacivirus, calicivirus, or coronavirus. Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375).

The self-replicating RNA may be associated with a delivery system. The self-replicating RNA may be administered with or without an adjuvant.

### RNA Delivery Systems

The self-replicating RNA of the invention are suitable for delivery in a variety of modalities, such as naked RNA delivery or in combination with lipids, polymers or other compounds that facilitate entry into the cells. Self-replicating RNA molecules of the present invention can be introduced into target cells or subjects using any suitable technique, e.g., by direct injection, microinjection, electroporation, lipofection, biolystics, and the like. The self-replicating RNA molecule may also be introduced into cells by way of receptor-mediated endocytosis. See e.g., U.S. Pat. No. 6,090,619; Wu and Wu, J. Biol. Chem., 263:14621 (1988); and Curiel et al., Proc. Natl. Acad. Sci. USA, 88:8850 (1991). For example, U.S. Pat. No. 6,083,741 discloses introducing an exogenous nucleic acid into mammalian cells by associating the nucleic acid to a polycation moiety (e.g., poly-L-lysine having 3-100 lysine residues), which is itself coupled to an integrin receptor-binding moiety (e.g., a cyclic peptide having the sequence Arg-Gly-Asp).

The self-replicating RNA molecule of the present invention can be delivered into cells via amphiphiles. See e.g., U.S. Pat. No. 6,071,890. Typically, a nucleic acid molecule may form a complex with the cationic amphiphile. Mammalian cells contacted with the complex can readily take it up.

The self-replicating RNA can be delivered as naked RNA (e.g. merely as an aqueous solution of RNA) but, to enhance entry into cells and also subsequent intercellular effects, the self-replicating RNA is preferably administered in combination with a delivery system, such as a particulate or emulsion delivery system. A large number of delivery systems are well known to those of skill in the art. Such delivery systems include, for example liposome-based delivery (Debs and Zhu (1993) WO 93/24640; Mannino and Gould-Fogerite (1988) BioTechniques 6(7): 682-691; Rose U.S. Pat. No. 5,279,833; Brigham (1991) WO 91/06309; and Felgner et al. (1987) Proc. Natl. Acad. Sci. USA 84: 7413-7414), as well as use of viral vectors (e.g., adenoviral (see, e.g., Berns et al. (1995) Ann. NY Acad. Sci. 772: 95-104; Ali et al. (1994) Gene Ther. 1: 367-384; and Haddada et al. (1995) Curr. Top. Microbiol. Immunol. 199 (Pt 3): 297-306 for review), papillomaviral, retroviral (see, e.g., Buchscher et al. (1992) J. Virol. 66(5) 2731-2739; Johann et al. (1992) J. Virol. 66 (5): 1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al., Gene Therapy (1994) supra.), and adeno-associated viral vectors (see, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 and Samulski (supra) for an overview of AAV vectors; see also, Lebkowski, U.S. Pat. No. 5,173,414; Tratschin et al. (1985) Mol. Cell. Biol. 5(11):3251-3260; Tratschin, et al. (1984) Mol. Cell. Biol., 4:2072-2081; Hermonat and Muzyczka (1984) Proc. Natl. Acad. Sci. USA, 81:6466-6470; McLaughlin et al. (1988) and Samulski et al. (1989) J. Virol., 63:03822-3828), and the like.

Three particularly useful delivery systems are (i) liposomes (ii) non-toxic and biodegradable polymer microparticles (iii) cationic submicron oil-in-water emulsions.

### Liposomes

Various amphiphilic lipids can form bilayers in an aqueous environment to encapsulate a RNA-containing aqueous core as a liposome. These lipids can have an anionic, cationic or zwitterionic hydrophilic head group. Formation of liposomes from anionic phospholipids dates back to the 1960s, and cationic liposome-forming lipids have been studied since the 1990s. Some phospholipids are anionic whereas other are zwitterionic. Suitable classes of phospholipid include, but are not limited to, phosphatidylethanolamines, phosphatidylcholines, phosphatidylserines, and phosphatidylglycerols, and some useful phospholipids are listed in Table 2. Useful cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are DPPC, DOPC and dodecylphosphocholine. The lipids can be saturated or unsaturated.

Liposomes can be formed from a single lipid or from a mixture of lipids. A mixture may comprise (i) a mixture of anionic lipids (ii) a mixture of cationic lipids (iii) a mixture of zwitterionic lipids (iv) a mixture of anionic lipids and cationic lipids (v) a mixture of anionic lipids and zwitterionic lipids (vi) a mixture of zwitterionic lipids and cationic lipids or (vii) a mixture of anionic lipids, cationic lipids and zwitterionic lipids. Similarly, a mixture may comprise both saturated and unsaturated lipids. For example, a mixture may comprise DSPC (zwitterionic, saturated), DlinDMA (cationic, unsaturated), and/or DMPG (anionic, saturated). Where a mixture of lipids is used, not all of the component lipids in the mixture need to be amphiphilic e.g. one or more amphiphilic lipids can be mixed with cholesterol.

The hydrophilic portion of a lipid can be PEGylated (i.e. modified by covalent attachment of a polyethylene glycol). This modification can increase stability and prevent non-specific adsorption of the liposomes. For instance, lipids can be conjugated to PEG using techniques such as those disclosed in Heyes et al. (2005) J Controlled Release 107:276-287.

A mixture of DSPC, DlinDMA, PEG-DMPG and cholesterol is used in the examples. A separate aspect of the invention is a liposome comprising DSPC, DlinDMA, PEG-DMG and cholesterol. This liposome preferably encapsulates RNA, such as a self-replicating RNA e.g. encoding an immunogen.

Liposomes are usually divided into three groups: multilamellar vesicles (MLV); small unilamellar vesicles (SUV); and large unilamellar vesicles (LUV). MLVs have multiple bilayers in each vesicle, forming several separate aqueous compartments. SUVs and LUVs have a single bilayer encapsulating an aqueous core; SUVs typically have a diameter ≤50 nm, and LUVs have a diameter >50 nm. Liposomes useful with of the invention are ideally LUVs with a diameter in the range of 50-220 nm. For a composition comprising a population of LUVs with different diameters: (i) at least 80% by number should have diameters in the range of 20-220 nm, (ii) the average diameter (Zav, by intensity) of the population is ideally in the range of 40-200 nm, and/or (iii) the diameters should have a polydispersity index <0.2.

Techniques for preparing suitable liposomes are well known in the art e.g. see Liposomes: Methods and Protocols, Volume 1: Pharmaceutical Nanocarriers: Methods and Protocols. (ed. Weissig). Humana Press, 2009. ISBN 160327359X; Liposome Technology, volumes I, II & III. (ed. Gregoriadis). Informa Healthcare, 2006; and Functional Polymer Colloids and Microparticles volume 4 (Microspheres, microcapsules & liposomes). (eds. Arshady & Guyot). Citus Books, 2002. One useful method involves mixing (i) an ethanolic solution of the lipids (ii) an aqueous solution of the nucleic acid and (iii) buffer, followed by mixing, equilibration, dilution and purification (Heyes et al. (2005) J Controlled Release 107:276-87).

RNA is preferably encapsulated within the liposomes, and so the liposome forms a outer layer around an aqueous RNA-containing core. This encapsulation has been found to protect RNA from RNase digestion. The liposomes can include some external RNA (e.g. on the surface of the liposomes), but at least half of the RNA (and ideally all of it) is encapsulated.

### Polymeric Microparticles

Various polymers can form microparticles to encapsulate or adsorb RNA. The use of a substantially non-toxic polymer means that a recipient can safely receive the particles, and the use of a biodegradable polymer means that the particles can be metabolised after delivery to avoid long-term persistence. Useful polymers are also sterilisable, to assist in preparing pharmaceutical grade formulations.

Suitable non-toxic and biodegradable polymers include, but are not limited to, poly(α-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

In some embodiments, the microparticles are formed from poly(α-hydroxy acids), such as a poly(lactides) (“PLA”), copolymers of lactide and glycolide such as a poly(D,L-lactide-co-glycolide) (“PLG”), and copolymers of D,L-lactide and caprolactone. Useful PLG polymers include those having a lactide/glycolide molar ratio ranging, for example, from 20:80 to 80:20 e.g. 25:75, 40:60, 45:55, 55:45, 60:40, 75:25. Useful PLG polymers include those having a molecular weight between, for example, 5,000-200,000 Da e.g. between 10,000-100,000, 20,000-70,000, 40,000-50,000 Da.

The microparticles ideally have a diameter in the range of 0.02 μm to 8 μm. For a composition comprising a population of microparticles with different diameters at least 80% by number should have diameters in the range of 0.03-7 μm.

Techniques for preparing suitable microparticles are well known in the art e.g. see Functional Polymer Colloids and Microparticles volume 4 (Microspheres, microcapsules & liposomes). (eds. Arshady & Guyot). Citus Books, 2002; Polymers in Drug Delivery. (eds. Uchegbu & Schatzlein). CRC Press, 2006. (in particular chapter 7) and Microparticulate Systems for the Delivery of Proteins and Vaccines. (eds. Cohen & Bernstein). CRC Press, 1996. To facilitate adsorption of RNA, a microparticle may include a cationic surfactant and/or lipid e.g. as disclosed in O'Hagan et al. (2001) J Virology 75:9037-9043; and Singh et al. (2003) Pharmaceutical Research 20: 247-251. An alternative way of making polymeric microparticles is by molding and curing e.g. as disclosed in WO2009/132206.

Microparticles of the invention can have a zeta potential of between 40-100 mV.

RNA can be adsorbed to the microparticles, and adsorption is facilitated by including cationic materials (e.g. cationic lipids) in the microparticle.

### Oil-In-Water Cationic Emulsions

Oil-in-water emulsions are known for adjuvanting influenza vaccines e.g. the MF59™ adjuvant in the FLUAD™ product, and the AS03 adjuvant in the PREPANDRIX™ product. RNA delivery according to the present invention can utilise an oil-in-water emulsion, provided that the emulsion includes one or more cationic molecules. For instance, a cationic lipid can be included in the emulsion to provide a positive droplet surface to which negatively-charged RNA can attach.

The emulsion comprises one or more oils. Suitable oil(s) include those from, for example, an animal (such as fish) or a vegetable source. The oil is ideally biodegradable (metabolisable) and biocompatible. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and so may be used. The procedures for separation, purification, saponification and other means necessary for obtaining pure oils from animal sources are well known in the art.

Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Squalane, the saturated analog to squalene, can also be used. Fish oils, including squalene and squalane, are readily available from commercial sources or may be obtained by methods known in the art.

Other useful oils are the tocopherols, particularly in combination with squalene. Where the oil phase of an emulsion includes a tocopherol, any of the α, β, γ, δ, ε or ξ tocopherols can be used, but α-tocopherols are preferred. D-α-tocopherol and DL-α-tocopherol can both be used. A preferred α-tocopherol is DL-α-tocopherol. An oil combination comprising squalene and a tocopherol (e.g. DL-α-tocopherol) can be used.

Preferred emulsions comprise squalene, a shark liver oil which is a branched, unsaturated terpenoid (C₃₀H₅₀; [(CH₃)₂C[═CHCH₂CH₂C(CH₃)]₂═CHCH₂₋]₂; 2,6,10,15,19,23-hexamethyl-2,6,10,14,18,22-tetracosahexaene; CAS RN 7683-64-9).

The oil in the emulsion may comprise a combination of oils e.g. squalene and at least one further oil.

The aqueous component of the emulsion can be plain water (e.g. w.f.i.) or can include further components e.g. solutes. For instance, it may include salts to form a buffer e.g. citrate or phosphate salts, such as sodium salts. Typical buffers include: a phosphate buffer; a Tris buffer; a borate buffer; a succinate buffer; a histidine buffer; or a citrate buffer. A buffered aqueous phase is preferred, and buffers will typically be included in the 5-20 mM range.

The emulsion also includes a cationic lipid. Preferably this lipid is a surfactant so that it can facilitate formation and stabilisation of the emulsion. Useful cationic lipids generally contains a nitrogen atom that is positively charged under physiological conditions e.g. as a tertiary or quaternary amine. This nitrogen can be in the hydrophilic head group of an amphiphilic surfactant. Useful cationic lipids include, but are not limited to: 1,2-dioleoyloxy-3-(trimethylammonio)propane (DOTAP), 3′-[N—(N′,N′-Dimethylaminoethane)-carbamoyl] Cholesterol (DC Cholesterol), dimethyldioctadecyl-ammonium (DDA e.g. the bromide), 1,2-Dimyristoyl-3-Trimethyl-AmmoniumPropane (DMTAP), dipalmitoyl(C16:0)trimethyl ammonium propane (DPTAP), distearoyltrimethylammonium propane (DSTAP). Other useful cationic lipids are: benzalkonium chloride (BAK), benzethonium chloride, cetramide (which contains tetradecyltrimethylammonium bromide and possibly small amounts of dedecyltrimethylammonium bromide and hexadecyltrimethyl ammonium bromide), cetylpyridinium chloride (CPC), cetyl trimethylammonium chloride (CTAC), N,N′,N′-polyoxyethylene (10)-N-tallow-1,3-diaminopropane, dodecyltrimethylammonium bromide, hexadecyltrimethyl-ammonium bromide, mixed alkyl-trimethylammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemetha-naminium chloride (DEBDA), dialkyldimetylammonium salts, [1-(2,3-dioleyloxy)-propyl]-N,N,N,trimethylammonium chloride, 1,2-diacyl-3-(trimethylammonio) propane (acyl group=dimyristoyl, dipalmitoyl, distearoyl, dioleoyl), 1,2-diacyl-3 (dimethylammonio)propane (acyl group=dimyristoyl, dipalmitoyl, distearoyl, dioleoyl), 1,2-dioleoyl-3-(4′-trimethyl-ammonio)butanoyl-sn-glycerol, 1,2-dioleoyl 3-succinyl-sn-glycerol choline ester, cholesteryl (4′-trimethylammonio) butanoate), N-alkyl pyridinium salts (e.g. cetylpyridinium bromide and cetylpyridinium chloride), N-alkylpiperidinium salts, dicationic bolaform electrolytes (C12Me6; C12BU6), dialkylglycetylphosphorylcholine, lysolecithin, L-α dioleoylphosphatidylethanolamine, cholesterol hemisuccinate choline ester, lipopolyamines, including but not limited to dioctadecylamidoglycylspermine (DOGS), dipalmitoyl phosphatidylethanol-amidospermine (DPPES), lipopoly-L (or D)-lysine (LPLL, LPDL), poly(L (or D)-lysine conjugated to N-glutarylphosphatidylethanolamine, didodecyl glutamate ester with pendant amino group (C\hat{ }GluPhCnN), ditetradecyl glutamate ester with pendant amino group (Cl4GIuCnN+), cationic derivatives of cholesterol, including but not limited to cholesteryl-3 β-oxysuccinamidoethylenetrimethylammonium salt, cholesteryl-3 β-oxysuccinamidoethylene-dimethylamine, cholesteryl-3 β-carboxyamidoethylenetrimethylammonium salt, and cholesteryl-3 β-carboxyamidoethylenedimethylamine. Other useful cationic lipids are described in US 2008/0085870 and US 2008/0057080, which are incorporated herein by reference.

The cationic lipid is preferably biodegradable (metabolisable) and biocompatible.

In addition to the oil and cationic lipid, an emulsion can include a non-ionic surfactant and/or a zwitterionic surfactant. Such surfactants include, but are not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); polyoxyethylene-9-lauryl ether; and sorbitan esters (commonly known as the Spans), such as sorbitan trioleate (Span 85) and sorbitan monolaurate. Preferred surfactants for including in the emulsion are polysorbate 80 (Tween 80; polyoxyethylene sorbitan monooleate), Span 85 (sorbitan trioleate), lecithin and Triton X-100.

Mixtures of these surfactants can be included in the emulsion e.g. Tween 80/Span 85 mixtures, or Tween 80/Triton-X100 mixtures. A combination of a polyoxyethylene sorbitan ester such as polyoxyethylene sorbitan monooleate (Tween 80) and an octoxynol such as t-octylphenoxy-polyethoxyethanol (Triton X-100) is also suitable. Another useful combination comprises laureth 9 plus a polyoxyethylene sorbitan ester and/or an octoxynol. Useful mixtures can comprise a surfactant with a HLB value in the range of 10-20 (e.g. polysorbate 80, with a HLB of 15.0) and a surfactant with a HLB value in the range of 1-10 (e.g. sorbitan trioleate, with a HLB of 1.8).

Preferred amounts of oil (% by volume) in the final emulsion are between 2-20% e.g. 5-15%, 6-14%, 7-13%, 8-12%. A squalene content of about 4-6% or about 9-11% is particularly useful.

Preferred amounts of surfactants (% by weight) in the final emulsion are between 0.001% and 8%. For example: polyoxyethylene sorbitan esters (such as polysorbate 80) 0.2 to 4%, in particular between 0.4-0.6%, between 0.45-0.55%, about 0.5% or between 1.5-2%, between 1.8-2.2%, between 1.9-2.1%, about 2%, or 0.85-0.95%, or about 1%; sorbitan esters (such as sorbitan trioleate) 0.02 to 2%, in particular about 0.5% or about 1%; octyl- or nonylphenoxy polyoxyethanols (such as Triton X-100) 0.001 to 0.1%, in particular 0.005 to 0.02%; polyoxyethylene ethers (such as laureth 9) 0.1 to 8%, preferably 0.1 to 10% and in particular 0.1 to 1% or about 0.5%.

The absolute amounts of oil and surfactant, and their ratio, can be varied within wide limits while still forming an emulsion. A skilled person can easily vary the relative proportions of the components to obtain a desired emulsion, but a weight ratio of between 4:1 and 5:1 for oil and surfactant is typical (excess oil).

An important parameter for ensuring immunostimulatory activity of an emulsion, particularly in large animals, is the oil droplet size (diameter). The most effective emulsions have a droplet size in the submicron range. Suitably the droplet sizes will be in the range 50-750 nm. Most usefully the average droplet size is less than 250 nm e.g. less than 200 nm, less than 150 nm. The average droplet size is usefully in the range of 80-180 nm. Ideally, at least 80% (by number) of the emulsion's oil droplets are less than 250 nm in diameter, and preferably at least 90%. Apparatuses for determining the average droplet size in an emulsion, and the size distribution, are commercially available. These typically use the techniques of dynamic light scattering and/or single-particle optical sensing e.g. the Accusizer™ and Nicomp™ series of instruments available from Particle Sizing Systems (Santa Barbara, USA), or the Zetasizer™ instruments from Malvern Instruments (UK), or the Particle Size Distribution Analyzer instruments from Horiba (Kyoto, Japan).

Ideally, the distribution of droplet sizes (by number) has only one maximum i.e. there is a single population of droplets distributed around an average (mode), rather than having two maxima. Preferred emulsions have a polydispersity of <0.4 e.g. 0.3, 0.2, or less.

Suitable emulsions with submicron droplets and a narrow size distribution can be obtained by the use of microfluidisation. This technique reduces average oil droplet size by propelling streams of input components through geometrically fixed channels at high pressure and high velocity. These streams contact channel walls, chamber walls and each other. The results shear, impact and cavitation forces cause a reduction in droplet size. Repeated steps of microfluidisation can be performed until an emulsion with a desired droplet size average and distribution are achieved.

As an alternative to microfluidisation, thermal methods can be used to cause phase inversion. These methods can also provide a submicron emulsion with a tight particle size distribution.

Preferred emulsions can be filter sterilized i.e. their droplets can pass through a 220 nm filter. As well as providing a sterilization, this procedure also removes any large droplets in the emulsion.

In certain embodiments, the cationic lipid in the emulsion is DOTAP. The cationic oil-in-water emulsion may comprise from about 0.5 mg/ml to about 25 mg/ml DOTAP. For example, the cationic oil-in-water emulsion may comprise DOTAP at from about 0.5 mg/ml to about 25 mg/ml, from about 0.6 mg/ml to about 25 mg/ml, from about 0.7 mg/ml to about 25 mg/ml, from about 0.8 mg/ml to about 25 mg/ml, from about 0.9 mg/ml to about 25 mg/ml, from about 1.0 mg/ml to about 25 mg/ml, from about 1.1 mg/ml to about 25 mg/ml, from about 1.2 mg/ml to about 25 mg/ml, from about 1.3 mg/ml to about 25 mg/ml, from about 1.4 mg/ml to about 25 mg/ml, from about 1.5 mg/ml to about 25 mg/ml, from about 1.6 mg/ml to about 25 mg/ml, from about 1.7 mg/ml to about 25 mg/ml, from about 0.5 mg/ml to about 24 mg/ml, from about 0.5 mg/ml to about 22 mg/ml, from about 0.5 mg/ml to about 20 mg/ml, from about 0.5 mg/ml to about 18 mg/ml, from about 0.5 mg/ml to about 15 mg/ml, from about 0.5 mg/ml to about 12 mg/ml, from about 0.5 mg/ml to about 10 mg/ml, from about 0.5 mg/ml to about 5 mg/ml, from about 0.5 mg/ml to about 2 mg/ml, from about 0.5 mg/ml to about 1.9 mg/ml, from about 0.5 mg/ml to about 1.8 mg/ml, from about 0.5 mg/ml to about 1.7 mg/ml, from about 0.5 mg/ml to about 1.6 mg/ml, from about 0.6 mg/ml to about 1.6 mg/ml, from about 0.7 mg/ml to about 1.6 mg/ml, from about 0.8 mg/ml to about 1.6 mg/ml, about 0.5 mg/ml, about 0.6 mg/ml, about 0.7 mg/ml, about 0.8 mg/ml, about 0.9 mg/ml, about 1.0 mg/ml, about 1.1 mg/ml, about 1.2 mg/ml, about 1.3 mg/ml, about 1.4 mg/ml, about 1.5 mg/ml, about 1.6 mg/ml, about 12 mg/ml, about 18 mg/ml, about 20 mg/ml, about 21.8 mg/ml, about 24 mg/ml, etc. In an exemplary embodiment, the cationic oil-in-water emulsion comprises from about 0.8 mg/ml to about 1.6 mg/ml DOTAP, such as 0.8 mg/ml, 1.2 mg/ml, 1.4 mg/ml or 1.6 mg/ml.

In certain embodiments, the cationic lipid is DC Cholesterol. The cationic oil-in-water emulsion may comprise DC Cholesterol at from about 0.1 mg/ml to about 5 mg/ml DC Cholesterol. For example, the cationic oil-in-water emulsion may comprise DC Cholesterol from about 0.1 mg/ml to about 5 mg/ml, from about 0.2 mg/ml to about 5 mg/ml, from about 0.3 mg/ml to about 5 mg/ml, from about 0.4 mg/ml to about 5 mg/ml, from about 0.5 mg/ml to about 5 mg/ml, from about 0.62 mg/ml to about 5 mg/ml, from about 1 mg/ml to about 5 mg/ml, from about 1.5 mg/ml to about 5 mg/ml, from about 2 mg/ml to about 5 mg/ml, from about 2.46 mg/ml to about 5 mg/ml, from about 3 mg/ml to about 5 mg/ml, from about 3.5 mg/ml to about 5 mg/ml, from about 4 mg/ml to about 5 mg/ml, from about 4.5 mg/ml to about 5 mg/ml, from about 0.1 mg/ml to about 4.92 mg/ml, from about 0.1 mg/ml to about 4.5 mg/ml, from about 0.1 mg/ml to about 4 mg/ml, from about 0.1 mg/ml to about 3.5 mg/ml, from about 0.1 mg/ml to about 3 mg/ml, from about 0.1 mg/ml to about 2.46 mg/ml, from about 0.1 mg/ml to about 2 mg/ml, from about 0.1 mg/ml to about 1.5 mg/ml, from about 0.1 mg/ml to about 1 mg/ml, from about 0.1 mg/ml to about 0.62 mg/ml, about 0.15 mg/ml, about 0.3 mg/ml, about 0.6 mg/ml, about 0.62 mg/ml, about 0.9 mg/ml, about 1.2 mg/ml, about 2.46 mg/ml, about 4.92 mg/ml, etc. In an exemplary embodiment, the cationic oil-in-water emulsion comprises from about 0.62 mg/ml to about 4.92 mg/ml DC Cholesterol, such as 2.46 mg/ml.

In certain embodiments, the cationic lipid is DDA. The cationic oil-in-water emulsion may comprise from about 0.1 mg/ml to about 5 mg/ml DDA. For example, the cationic oil-in-water emulsion may comprise DDA at from about 0.1 mg/ml to about 5 mg/ml, from about 0.1 mg/ml to about 4.5 mg/ml, from about 0.1 mg/ml to about 4 mg/ml, from about 0.1 mg/ml to about 3.5 mg/ml, from about 0.1 mg/ml to about 3 mg/ml, from about 0.1 mg/ml to about 2.5 mg/ml, from about 0.1 mg/ml to about 2 mg/ml, from about 0.1 mg/ml to about 1.5 mg/ml, from about 0.1 mg/ml to about 1.45 mg/ml, from about 0.2 mg/ml to about 5 mg/ml, from about 0.3 mg/ml to about 5 mg/ml, from about 0.4 mg/ml to about 5 mg/ml, from about 0.5 mg/ml to about 5 mg/ml, from about 0.6 mg/ml to about 5 mg/ml, from about 0.73 mg/ml to about 5 mg/ml, from about 0.8 mg/ml to about 5 mg/ml, from about 0.9 mg/ml to about 5 mg/ml, from about 1.0 mg/ml to about 5 mg/ml, from about 1.2 mg/ml to about 5 mg/ml, from about 1.45 mg/ml to about 5 mg/ml, from about 2 mg/ml to about 5 mg/ml, from about 2.5 mg/ml to about 5 mg/ml, from about 3 mg/ml to about 5 mg/ml, from about 3.5 mg/ml to about 5 mg/ml, from about 4 mg/ml to about 5 mg/ml, from about 4.5 mg/ml to about 5 mg/ml, about 1.2 mg/ml, about 1.45 mg/ml, etc. Alternatively, the cationic oil-in-water emulsion may comprise DDA at about 20 mg/ml, about 21 mg/ml, about 21.5 mg/ml, about 21.6 mg/ml, about 25 mg/ml. In an exemplary embodiment, the cationic oil-in-water emulsion comprises from about 0.73 mg/ml to about 1.45 mg/ml DDA, such as 1.45 mg/ml.

Catheters or like devices may be used to deliver the self-replicating RNA molecules of the invention, as naked RNA or in combination with a delivery system, into a target organ or tissue. Suitable catheters are disclosed in, e.g., U.S. Pat. Nos. 4,186,745; 5,397,307; 5,547,472; 5,674,192; and 6,129,705, all of which are incorporated herein by reference.

The present invention includes the use of suitable delivery systems, such as liposomes, polymer microparticles or submicron emulsion microparticles with encapsulated or adsorbed self-replicating RNA, to deliver a self-replicating RNA molecule that encodes an RSV-F polypeptide, for example, to elicit an immune response alone, or in combination with another macromolecule. The invention includes liposomes, microparticles and submicron emulsions with adsorbed and/or encapsulated self-replicating RNA molecules, and combinations thereof.

As demonstrated further in the Examples, the self-replicating RNA molecules associated with liposomes and submicron emulsion microparticles can be effectively delivered to the host cell, and can induce an immune response to the protein encoded by the self-replicating RNA.

### Immunogenic Compositions

The invention provides immunogenic compositions. The immunogenic compositions may include a single active immunogenic agent, or several immunogenic agents. For example, the immunogenic composition can comprise pre-fusion RSV F polypeptides or a combination of pre-fusion chimeric RSV F polypeptides. The immunogenic composition can comprise a self-replicating RNA encoding a pre-fusion RSV-F polypeptide, and preferably also comprises a suitable delivery system, such as liposomes, polymeric microparticles, an oil-in-water emulsion and combinations thereof.

Immunogenic compositions of the invention may also comprise one or more immunoregulatory agents. Preferably, one or more of the immunoregulatory agents include one or more adjuvants, for example two, three, four or more adjuvants. The adjuvants may include a TH1 adjuvant and/or a TH2 adjuvant, further discussed below.

In another embodiment, an immunogenic composition of the invention comprises a polypeptide that displays an epitope present in a pre-fusion conformation of RSV-F glycoprotein.

In another embodiment, an immunogenic composition of the invention comprises one or more pre-fusion chimera proteins based on two different pre-fusion non-RSV (F proteins (e.g, metapneumovirus, parainfluenza, such asPIV5, NDV), in which both have the same RSV F neutralizing epitopes mutated on the protein surface.

The compositions of the invention are preferably suitable for administration to a mammalian subject, such as a human, and include one or more pharmaceutically acceptable carrier(s) and/or excipient(s), including adjuvants. A thorough discussion of such components is available in reference 29. Compositions will generally be in aqueous form. When the composition is an immunogenic composition, it will elicit an immune response when administered to a mammal, such as a human. The immunogenic composition can be used to prepare a vaccine formulation for immunizing a mammal.

The immunogenic compositions may include a single active immunogenic agent, or several immunogenic agents. For example, the pre-fusion RSV F protein can be full length or a ecto-domain polypeptide and can be in a single form (e.g., uncleaved monomer, cleaved monomer, uncleaved trimer, cleaved trimer) or in two or more forms (e.g., a mixture of uncleaved monomer and uncleaved trimer or a dynamic equilibrium between uncleaved monomer and uncleaved trimer). In addition, the compositions can contain a pre-fusion RSV F protein and one or more other RSV proteins (e.g., a G protein and/or an M protein) and/or it may be combined with immunogens from other pathogens.

The composition may include preservatives such as thiomersal or 2-phenoxyethanol. It is preferred, however, that the vaccine should be substantially free from (i.e., less than 5 μg/ml) mercurial material, e.g., thiomersal-free. Immunogenic compositions containing no mercury are more preferred. Preservative-free immunogenic compositions are particularly preferred.

To control tonicity, it is preferred to include a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride, calcium chloride, and the like.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range. The pH of a composition will generally be between 5.0 and 8.1, and more typically between 6.0 and 8.0, e.g., between 6.5 and 7.5, or between 7.0 and 7.8. A process of the invention may therefore include a step of adjusting the pH of the bulk vaccine prior to packaging.

The composition is preferably sterile. The composition is preferably non-pyrogenic, e.g., containing <1 EU (endotoxin unit, a standard measure) per dose, and preferably <0.1 EU per dose. The composition is preferably gluten free. Human vaccines are typically administered in a dosage volume of about 0.5 ml, although a half dose (i.e., about 0.25 ml) may be administered to children.

### Adjuvants

Compositions of the invention, that contain RSV-F polypeptides, or nucleic acids that encode RSV-F polypeptides, may also include one or more adjuvants, for example two, three, four or more adjuvants, which can function to enhance the immune responses (humoral and/or cellular) elicited in a patient who receives the composition. The adjuvants may include a TH1 adjuvant and/or a TH2 adjuvant. Adjuvants which may be used in compositions of the invention include, but are not limited to:
Mineral-containing compositions. Mineral-containing compositions suitable for use as adjuvants in the invention include mineral salts, such as calcium salts and aluminum salts (or mixtures thereof). The invention includes mineral salts such as hydroxides (e.g. oxyhydroxides), phosphates (e.g. hydroxyphosphates, orthophosphates), sulphates, etc., or mixtures of different mineral compounds, with the compounds taking any suitable form (e.g. gel, crystalline, amorphous, etc.), and with adsorption being preferred. Calcium salts include calcium phosphate (e.g., the “CAP” particles disclosed in ref. 38). Aluminum salts include hydroxides, phosphates, sulfates, and the like. The mineral containing compositions may also be formulated as a particle of metal salt (39). Aluminum salt adjuvants are described in more detail below.
Oil emulsion compositions (see in more detail below). Oil emulsion compositions suitable for use as adjuvants in the invention include squalene-water emulsions, such as MF59 (5% Squalene, 0.5% Tween 80 and 0.5% Span, formulated into submicron particles using a microfluidizer).
Cytokine-inducing agents (see in more detail below). Cytokine-inducing agents suitable for use in the invention include toll-like receptor 7 (TLR7) agonists (e.g. benzonaphthyridine compounds disclosed in WO 2009/111337.
Saponins (chapter 22 of ref. 74), which are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as STIMULON (TM). Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 40. Saponin formulations may also comprise a sterol, such as cholesterol (41). Combinations of saponins and cholesterols can be used to form unique particles called immunostimulating complexes (ISCOMs) (chapter 23 of ref. 74). ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 41-43. Optionally, the ISCOMS may be devoid of additional detergent (44). A review of the development of saponin based adjuvants can be found in refs. 45 & 46.
Fatty adjuvants (see in more detail below), including oil-in-water emulsions, modified natural lipid As derived from enterobacterial lipopolysaccharides, phospholipid compounds (such as the synthetic phospholipid dimer, E6020) and the like.
Bacterial ADP-ribosylating toxins (e.g., the E. coli heat labile enterotoxin “LT”, cholera toxin “CT”, or pertussis toxin “PT”) and detoxified derivatives thereof, such as the mutant toxins known as LT-K63 and LT-R72 (47). The use of detoxified ADP-ribosylating toxins as mucosal adjuvants is described in ref. 48 and as parenteral adjuvants in ref. 49.
Bioadhesives and mucoadhesives, such as esterified hyaluronic acid microspheres (50) or chitosan and its derivatives (51).
Microparticles (i.e., a particle of ˜100 nm to ˜150 m in diameter, more preferably ˜200 nm to ˜30 m in diameter, or ˜500 nm to ˜10 m in diameter) formed from materials that are biodegradable and non-toxic (e.g., a poly(α-hydroxy acid), a polyhydroxybutyric acid, a polyorthoester, a polyanhydride, a polycaprolactone, and the like), with poly(lactide-co-glycolide) being preferred, optionally treated to have a negatively-charged surface (e.g., with SDS) or a positively-charged surface (e.g., with a cationic detergent, such as CTAB).
Liposomes (Chapters 13 & 14 of ref. 74). Examples of liposome formulations suitable for use as adjuvants are described in refs. 52-54.
Polyoxyethylene ethers and polyoxyethylene esters (55). Such formulations further include polyoxyethylene sorbitan ester surfactants in combination with an octoxynol (56) as well as polyoxyethylene alkyl ethers or ester surfactants in combination with at least one additional non-ionic surfactant such as an octoxynol (57). Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.
Muramyl peptides, such as N-acetylmuramyl-L-threonyl-D-isoglutamine (“thr-MDP”), N-acetyl-normuramyl-L-alanyl-D-isoglutamine (nor-MDP), N-acetylglucsaminyl-N-acetylmuramyl-L-Al-D-isoglu-L-Ala-dipalmitoxy propylamide (“DTP-DPP”, or “Theramide™), N-acetylmuramyl-L-alanyl-D-isoglutaminyl-L-alanine-2-(1′-2′dipalmitoyl-sn-glycero-3-hydroxyphosphoryloxy)-ethylamine (“MTP-PE”).
An outer membrane protein proteosome preparation prepared from a first Gram-negative bacterium in combination with a liposaccharide preparation derived from a second Gram-negative bacterium, wherein the outer membrane protein proteosome and liposaccharide preparations form a stable non-covalent adjuvant complex. Such complexes include “IVX-908”, a complex comprised of Neisseria meningitidis outer membrane and lipopolysaccharides.
A polyoxidonium polymer (58, 59) or other N-oxidized polyethylene-piperazine derivative.
Methyl inosine 5′-monophosphate (“MIMIP”) (60).
A polyhydroxlated pyrrolizidine compound (61), such as one having formula:

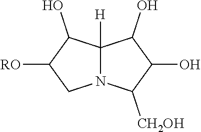

where R is selected from the group comprising hydrogen, straight or branched, unsubstituted or substituted, saturated or unsaturated acyl, alkyl (e.g., cycloalkyl), alkenyl, alkynyl and aryl groups, or a pharmaceutically acceptable salt or derivative thereof. Examples include, but are not limited to: casuarine, casuarine-6-α-D-glucopyranose, 3-epi-casuarine, 7-epi-casuarine, 3,7-diepi-casuarine, and the like
A CD1d ligand, such as an α-glycosylceramide (62-69) (e.g., α-galactosylceramide), phytosphingosine-containing α-glycosylceramides, OCH, KRN7000 [(2S,3S,4R)-1-O-(α-D-galactopyranosyl)-2-(N-hexacosanoylamino)-1,3,4-octadecanetriol], CRONY-101, 3″-O-sulfo-galactosylceramide, etc.
A gamma inulin (70) or derivative thereof, such as algammulin.
Virosomes and virus-like particles (VLPs). These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qß-phage (such as coat proteins), GA-phage, fr-phage, AP205 phage, and Ty (such as retrotransposon Ty protein p1).

These and other adjuvant-active substances are discussed in more detail in references 74 & 75.

Compositions may include two, three, four or more adjuvants. For example, compositions of the invention may advantageously include both an oil-in-water emulsion and a cytokine-inducing agent, or both a mineral-containing composition and a cytokine-inducing agent, or two oil-in-water emulsion adjuvants, or two benzonaphthyridine compounds, etc.

Antigens and adjuvants in a composition will typically be in admixture.

### Oil Emulsion Adjuvants

Oil emulsion compositions suitable for use as adjuvants in the invention include squalene-water emulsions, such as MF59 (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

Various oil-in-water emulsions are known, and they typically include at least one oil and at least one surfactant, with the oil(s) and surfactant(s) being biodegradable (metabolizable) and biocompatible. The oil droplets in the emulsion are generally less than 5 μm in diameter, and may even have a sub-micron diameter, with these small sizes being achieved with a microfluidizer to provide stable emulsions. Droplets with a size less than 220 nm are preferred as they can be subjected to filter sterilization.

The invention can be used with oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used, e.g., obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoid known as squalene, 2,6,10,15,19,23-hexamethyl-2,6,10,14,18,22-tetracosahexaene, which is particularly preferred herein. Squalane, the saturated analog to squalene, is also preferred oil. Fish oils, including squalene and squalane, are readily available from commercial sources or may be obtained by methods known in the art. Other preferred oils are the tocopherols (see below). Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX (TM) tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the TERGITOL (TM) NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. Preferred surfactants for including in the emulsion are TWEEN 80 (TM) (polyoxyethylene sorbitan monooleate), Span 85 (sorbitan trioleate), lecithin and Triton X-100.

Mixtures of surfactants can be used e.g., TWEEN 80 (TM)/Span 85 mixtures. A combination of a polyoxyethylene sorbitan ester such as polyoxyethylene sorbitan monooleate (TWEEN 80 (TM)) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as TWEEN 80 (TM)) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Specific oil-in-water emulsion adjuvants useful with the invention include, but are not limited to:
A submicron emulsion of squalene, TWEEN 80 (TM), and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ (71-73), as described in more detail in Chapter 10 of ref. 74 and chapter 12 of ref. 75. The MF59 emulsion advantageously includes citrate ions, e.g., 10 mM sodium citrate buffer.
An emulsion of squalene, a tocopherol, and TWEEN 80 (TM). The emulsion may include phosphate buffered saline. It may also include Span 85 (e.g., at 1%) and/or lecithin. These emulsions may have from 2 to 10% squalene, from 2 to 10% tocopherol and from 0.3 to 3% TWEEN 80 (TM), and the weight ratio of squalene:tocopherol is preferably <1 as this provides a more stable emulsion. Squalene and TWEEN 80 (TM) may be present volume ratio of about 5:2. One such emulsion can be made by dissolving TWEEN 80 (TM) in PBS to give a 2% solution, then mixing 90 ml of this solution with a mixture of (5 g of DL-α-tocopherol and 5 ml squalene), then microfluidizing the mixture. The resulting emulsion may have submicron oil droplets, e.g., with an average diameter of between 100 and 250 nm, preferably about 180 nm.
An emulsion of squalene, a tocopherol, and a Triton detergent (e.g., Triton X-100). The emulsion may also include a 3d-MPL (see below). The emulsion may contain a phosphate buffer.
An emulsion comprising a polysorbate (e.g., polysorbate 80), a Triton detergent (e.g., Triton X-100) and a tocopherol (e.g., an α-tocopherol succinate). The emulsion may include these three components at a mass ratio of about 75:11:10 (e.g., 750 g/ml polysorbate 80, 110 g/ml Triton X-100 and 100 g/ml α-tocopherol succinate), and these concentrations should include any contribution of these components from antigens. The emulsion may also include squalene. The emulsion may also include a 3d-MPL (see below). The aqueous phase may contain a phosphate buffer.
An emulsion of squalane, polysorbate 80 and poloxamer 401 (“PLURONIC (TM) L121”). The emulsion can be formulated in phosphate buffered saline, pH 7.4. This emulsion is a useful delivery vehicle for muramyl dipeptides, and has been used with threonyl-MDP in the “SAF-1” adjuvant (76) (0.05-1% Thr-MDP, 5% squalane, 2.5% Pluronic L121 and 0.2% polysorbate 80). It can also be used without the Thr-MDP, as in the “AF” adjuvant (77) (5% squalane, 1.25% Pluronic L121 and 0.2% polysorbate 80). Microfluidization is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm. The emulsion may also include one or more of: alditol; a cryoprotective agent (e.g. a sugar, such as dodecylmaltoside and/or sucrose); and/or an alkylpolyglycoside. Such emulsions may be lyophilized.
An emulsion having from 0.5-50% of an oil, 0.1-10% of a phospholipid, and 0.05-5% of a non-ionic surfactant. As described in reference 78, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolizable oil (such as light mineral oil) and at least one surfactant (such as lecithin, TWEEN 80 (TM) or Span 80). Additives may be included, such as QuilA saponin, cholesterol, a saponin-lipophile conjugate (such as GPI-0100, described in reference 79, produced by addition of aliphatic amine to desacylsaponin via the carboxyl group of glucuronic acid), dimethyidioctadecylammonium bromide and/or N,N-dioctadecyl-N,N-bis(2-hydroxyethyl)propanediamine.
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer).
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer).
An emulsion in which a saponin (e.g., QuilA or QS21) and a sterol (e.g., a cholesterol) are associated as helical micelles (80).

The emulsions may be mixed with antigen extemporaneously, at the time of delivery. Thus the adjuvant and antigen may be kept separately in a packaged or distributed vaccine, ready for final formulation at the time of use. The antigen will generally be in an aqueous form, such that the vaccine is finally prepared by mixing two liquids. The volume ratio of the two liquids for mixing can vary (e.g., between 5:1 and 1:5) but is generally about 1:1.

### Cytokine-Inducing Agents

Cytokine-inducing agents for inclusion in compositions of the invention are able, when administered to a patient, to elicit the immune system to release cytokines, including interferons and interleukins. Preferred agents can elicit the release of one or more of: interferon-γ; interleukin-1; interleukin-2; interleukin-12; TNF-α; TNF-β; and GM-CSF. Preferred agents elicit the release of cytokines associated with a Th1-type immune response, e.g., interferon-γ, TNF-α, interleukin-2. Stimulation of both interferon-γ and interleukin-2 is preferred.

As a result of receiving a composition of the invention, therefore, a patient will have T cells that, when stimulated with a RSV F protein, will release the desired cytokine(s) in an antigen-specific manner. For example, T cells purified from their blood will release 7-interferon when exposed in vitro to F protein. Methods for measuring such responses in peripheral blood mononuclear cells (PBMC) are known in the art, and include ELISA, ELISPOT, flow-cytometry and real-time PCR. For example, reference 81 reports a study in which antigen-specific T cell-mediated immune responses against tetanus toxoid, specifically 7-interferon responses, were monitored, and found that ELISPOT was the most sensitive method to discriminate antigen-specific TT-induced responses from spontaneous responses, but that intracytoplasmic cytokine detection by flow cytometry was the most efficient method to detect re-stimulating effects.

Suitable cytokine-inducing agents include, but are not limited to:
An immunostimulatory oligonucleotide, such as one containing a CpG motif (a dinucleotide sequence containing an unmethylated cytosine linked by a phosphate bond to a guanosine), or a double-stranded RNA, or an oligonucleotide containing a palindromic sequence, or an oligonucleotide containing a poly(dG) sequence.
3-O-deacylated monophosphoryl lipid A (‘3dMPL’, also known as ‘MPL (TM)’) (82-85).
An imidazoquinoline compound, such as IMIQUIMOD (TM) (“R-837”) (86, 87), RESIQUIMOD (TM) (“R-848”) (88), and their analogs; and salts thereof (e.g., the hydrochloride salts). Further details about immunostimulatory imidazoquinolines can be found in references 89 to 93.
A benzonaphthyridine compound, such as: (a) a compound having the formula:

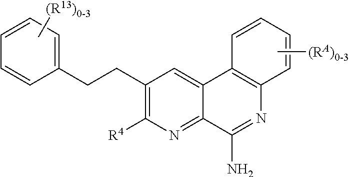

wherein:
   R⁴ is selected from H, halogen, —C(O)OR⁷, —C(O)R⁷, —C(O)N(R¹¹R¹²),
      —N(R¹¹R¹²), —N(R⁹)₂, —NHN(R⁹)₂, —SR⁷, —(CH₂), OR⁷, —(CH₂), R⁷,
      -LR⁸, -LR¹⁰, -OLR⁸, -OLR¹⁰, C₁-C₆alkyl, C₁-C₆heteroalkyl,
      C₁-C₆haloalkyl, C₂-C₈alkene, C₂-C₈alkyne, C₁-C₆alkoxy,
      C₁-C₆haloalkoxy, aryl, heteroaryl, C₃-C₈cycloalkyl, and
      C₃-C₈heterocycloalkyl, wherein the C₁-C₆alkyl, C₁-C₆heteroalkyl, C₁-C₆haloalkyl, C₂-C₈alkene, C₂-C₈alkyne, C₁-C₆alkoxy,
      C₁-C₆haloalkoxy, aryl, heteroaryl, C₃-C₈cycloalkyl, and
      C₃-C₈heterocycloalkyl groups of R⁴ are each optionally substituted with 1 to 3 substituents independently selected from halogen, —CN, —NO₂, —R⁷, —OR⁸, —C(O)R⁸, —OC(O)R⁸, —C(O)OR⁸, —N(R⁹)₂, —P(O)(OR⁸)₂, —OP(O)(OR⁸)₂, —P(O)(OR¹⁰)₂
      —OP(O)(OR¹⁰)₂, —C(O)N(R⁹)₂, —S(O)₂R⁸, —S(O)R⁸, —S(O)₂N(R⁹)₂, and —NR⁹S(O)₂R⁸;
   each L is independently selected from a bond, —(O(CH₂)ₘ)ₜ—, C₁-C₆alkyl,
      C₂-C₆alkenylene and C₂-C₆alkynylene, wherein the C₁-C₆alkyl,
      C₂-C₆alkenylene and C₂-C₆alkynylene of L are each optionally substituted with 1 to 4 substituents independently selected from halogen, —R⁸, —OR⁸, —N(R⁹)₂, —P(O)(OR⁸)₂, —OP(O)(OR⁸)₂,
      —P(O)(OR¹⁰)₂, and —OP(O)(OR¹⁰)₂;
   R⁷ is selected from H, C₁-C₆alkyl, aryl, heteroaryl, C₃-C₈cycloalkyl,
      C₁-C₆heteroalkyl, C₁-C₆haloalkyl, C₂-C₅alkene, C₂-C₅alkyne,
      C₁-C₆alkoxy, C₁-C₆haloalkoxy, and C₃-C₅heterocycloalkyl, wherein the
      C₁-C₆alkyl, aryl, heteroaryl, C₃-C₈cycloalkyl,
      C₁-C₆heteroalkyl, C₁-C₆haloalkyl, C₂-C₅alkene, C₂-C₅alkyne,
      C₁-C₆alkoxy, C₁-C₆haloalkoxy, and C₃-C₈heterocycloalkyl groups of R⁷ are each optionally substituted with 1 to 3 R¹³ groups;
   each R⁸ is independently selected from H, —CH(R¹⁰)₂, C₁-C₅alkyl,
      C₂-C₈alkene, C₂-C₈alkyne, C₁-C₆haloalkyl, C₁-C₆alkoxy,
      C₁-C₆heteroalkyl, C₃-C₅cycloalkyl, C₂-C₅heterocycloalkyl,
      C₁-C₆hydroxyalkyl and C₁-C₆haloalkoxy, wherein the C₁-C₈alkyl, C₂-C₈alkene, C₂-C₅alkyne, C₁-C₆heteroalkyl, C₁-C₆haloalkyl,
      C₁-C₆alkoxy, C₃-C₈cycloalkyl, C₂-C₈heterocycloalkyl,
      C₁-C₆hydroxyalkyl and C₁-C₆haloalkoxy groups of R⁸ are each optionally substituted with 1 to 3 substituents independently selected from —CN, R¹¹, —OR¹¹, —SR¹¹, —C(O)R¹¹, —OC(O)R¹¹,
      —C(O)N(R⁹)₂, —C(O)OR¹¹, —NR⁹C(O)R¹¹, —NR⁹R¹⁰, —NR¹¹R¹², —N(R⁹)₂, —OR⁹, —OR¹⁰, —C(O)NR¹¹R¹², —C(O)NR¹¹OH, —S(O)₂R¹¹,
      —S(O)R¹¹, —S(O)₂NR¹¹R¹², —NR¹¹S(O)₂R¹¹, —P(O)(OR¹¹)₂, and
      —OP(O)(OR¹¹)₂;
   each R⁹ is independently selected from H, —C(O)R⁸, —C(O)OR⁸, —C(O)R¹⁰,
      —C(O)OR¹⁰, —S(O)₂R¹⁰, —C₁-C₆ alkyl, C₁-C₆ heteroalkyl and C₃-C₆ cycloalkyl, or each R⁹ is independently a C₁-C₆alkyl that together with N they are attached to form a C₃-C₅heterocycloalkyl, wherein the C₃-C₈heterocycloalkyl ring optionally contains an additional heteroatom selected from N, O and S, and wherein the C₁-C₆ alkyl, C₁-C₆ heteroalkyl, C₃-C₆ cycloalkyl, or
      C₃-C₈heterocycloalkyl groups of R⁹ are each optionally substituted with 1 to 3 substituents independently selected from
      —CN, R¹¹, —OR¹¹, —SR¹¹, —C(O)R¹¹, —OC(O)R¹¹, —C(O)OR¹¹, —NR¹¹R¹², —C(O)NR¹¹R¹², —C(O)NR¹¹OH, —S(O)₂R¹¹, —S(O)R¹¹,
      —S(O)₂NR¹¹R¹², —NR¹¹S(O)₂R¹¹, —P(O)(OR¹)₂, and
      —OP(O)(OR¹¹)₂;
   each R¹⁰ is independently selected from aryl, C₃-C₈cycloalkyl,
      C₃-C₈heterocycloalkyl and heteroaryl, wherein the aryl,
      C₃-C₅cycloalkyl, C₃-C₅heterocycloalkyl and heteroaryl groups are optionally substituted with 1 to 3 substituents selected from halogen, —R⁸, —OR⁸, -LR⁹, -LOR⁹, —N(R⁹)₂, —NR⁹C(O)R⁸,
      —NR⁹CO₂R⁸, —CO₂R⁸, —C(O)R⁸ and —C(O)N(R⁹)₂;
   R¹¹ and R¹² are independently selected from H, C₁-C₆alkyl,
      C₁-C₆heteroalkyl, C₁-C₆haloalkyl, aryl, heteroaryl,
      C₃-C₈cycloalkyl, and C₃-C₈heterocycloalkyl, wherein the
      C₁-C₆alkyl, C₁-C₆heteroalkyl, C₁-C₆haloalkyl, aryl, heteroaryl, C₃-C₈cycloalkyl, and C₃-C₅heterocycloalkyl groups of R¹¹ and R¹² are each optionally substituted with 1 to 3 substituents independently selected from halogen, —CN, R⁸, —OR⁸, —C(O)R⁸,
      —OC(O)R⁸, —C(O)OR⁸, —N(R⁹)₂, —NR⁸C(O)R⁸, —NR⁸C(O)OR⁸,
      —C(O)N(R⁹)₂, C₃-C₈heterocycloalkyl, —S(O)₂R⁸, —S(O)₂N(R⁹)₂,
      —NR⁹S(O)₂R⁸, C₁-C₆haloalkyl and C₁-C₆haloalkoxy;
   or R¹¹ and R¹² are each independently C₁-C₆alkyl and taken together with the N atom to which they are attached form an optionally substituted C₃-C₈heterocycloalkyl ring optionally containing an additional heteroatom selected from N, O and S;
   each R¹³ is independently selected from halogen, —CN, -LR⁹, -LOR⁹,
      -OLR⁹, -LR¹⁰, -LOR¹⁰, -OLR¹⁰, -LR⁸, -LOR⁸, -OLR⁸, -LSR⁸,
      -LSR¹⁰, -LC(O)R⁸, -OLC(O)R⁸, -LC(O)OR⁸, -LC(O)R¹⁰,
      -LOC(O)OR⁸, -LC(O)NR⁹R¹¹, -LC(O)NR⁹R⁸, -LN(R⁹)₂,
      -LNR⁹R⁸, -LNR⁹R¹⁰, -LC(O)N(R⁹)₂, -LS(O)₂R⁸, -LS(O)R⁸,
      -LC(O)NR⁸OH, -LNR⁹C(O)R⁸, -LNR⁹C(O)OR⁸, -LS(O)₂N(R⁹)₂, -OLS(O)₂N(R⁹)₂, -LNR⁹S(O)₂R⁸, -LC(O)NR⁹LN(R⁹)₂,
      -LP(O)(OR⁸)₂, -LOP(O)(OR⁸)₂, -LP(O)(OR¹⁰)₂ and
      -OLP(O)(OR¹⁰)₂;
   each R^{A} is independently selected from —R⁸, —R⁷, —OR⁷, —OR⁸, —R¹⁰, —OR¹⁰, —SR⁸, —NO₂, —CN, —N(R⁹)₂, —NR⁹C(O)R⁸, —NR⁹C(S)R⁸, —NR⁹C(O)N(R⁹)₂, —NR⁹C(S)N(R⁹)₂, —NR⁹CO₂R⁸, —NR⁹NR⁹C(O)R⁸, —NR⁹NR⁹C(O)N(R⁹)₂, —NR⁹NR⁹CO₂R⁸, —C(O)C(O)R⁸, —C(O)CH₂C(O)R⁸, —CO₂R⁸, —(CH₂), CO₂R⁸, —C(O)R⁸, —C(S)R⁸, —C(O)N(R⁹)₂, —C(S)N(R⁹)₂, —OC(O)N(R⁹)₂, —OC(O)R⁸, —C(O)N(OR⁸)R⁸, —C(NOR⁸)R⁸, —S(O)₂R⁸, —S(O)₃R⁸, —SO₂N(R⁹)₂, —S(O)R⁸, —NR⁹SO₂N(R⁹)₂, —NR⁹SO₂R⁸, —P(O)(OR⁸)₂, —OP(O)(OR⁸)₂, —P(O)(OR¹⁰)₂, —OP(O)(OR¹⁰)₂, —N(OR⁸)R⁸, —CH═CHCO₂R⁸, —C(═NH)—N(R⁹)₂, and —(CH₂)ₙNHC(O)R⁸; or two adjacent R^{A} substituents on Ring A form a 5-6 membered ring that contains up to two heteroatoms as ring members;
n is, independently at each occurrence, 0, 1, 2, 3, 4, 5, 6, 7 or 8; each m is independently selected from 1, 2, 3, 4, 5 and 6, and
t is 1, 2, 3, 4, 5, 6, 7 or 8; (b) a compound having the formula:

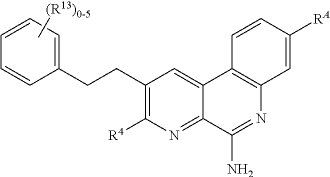

wherein:
   R⁴ is selected from H, halogen, —C(O)OR⁷, —C(O)R⁷, —C(O)N(R¹¹R¹²),
      —N(R¹¹R¹²), —N(R⁹)₂, —NHN(R⁹)₂, —SR⁷, —(CH₂), OR⁷, —(CH₂), R⁷,
      -LR⁸, -LR¹⁰, -OLR⁸, -OLR¹⁰, C₁-C₆alkyl, C₁-C₆heteroalkyl,
      C₁-C₆haloalkyl, C₂-C₈alkene, C₂-C₈alkyne, C₁-C₆alkoxy,
      C₁-C₆haloalkoxy, aryl, heteroaryl, C₃-C₈cycloalkyl, and C₃-C₈heterocycloalkyl, wherein the C₁-C₆alkyl, C₁-C₆heteroalkyl, C₁-C₆haloalkyl, C₂-C₈alkene, C₂-C₈alkyne, C₁-C₆alkoxy,
      C₁-C₆haloalkoxy, aryl, heteroaryl, C₃-C₈cycloalkyl, and C₃-C₈heterocycloalkyl groups of R⁴ are each optionally substituted with 1 to 3 substituents independently selected from halogen, —CN, —NO₂, —R⁷, —OR⁸, —C(O)R⁸, —OC(O)R⁸, —C(O)OR⁸, —N(R⁹)₂, —P(O)(OR⁸)₂, —OP(O)(OR⁸)₂, —P(O)(OR¹⁰)₂
      —OP(O)(OR¹⁰)₂, —C(O)N(R⁹)₂, —S(O)₂R⁸, —S(O)R⁸,
      —S(O)₂N(R⁹)₂, and —NR⁹S(O)₂R⁸;
   each L is independently selected from a bond, —(O(CH₂)ₘ)ₜ—, C₁-C₆alkyl, C₂-C₆alkenylene and C₂-C₆alkynylene, wherein the C₁-C₆alkyl, C₂-C₆alkenylene and C₂-C₆alkynylene of L are each optionally substituted with 1 to 4 substituents independently selected from halogen, —R⁸, —OR⁸, —N(R⁹)₂, —P(O)(OR⁸)₂, —OP(O)(OR⁸)₂,
      —P(O)(OR¹⁰)₂, and —OP(O)(OR¹⁰)₂;
   R⁷ is selected from H, C₁-C₆alkyl, aryl, heteroaryl, C₃-C₈cycloalkyl,
      C₁-C₆heteroalkyl, C₁-C₆haloalkyl, C₂-C₅alkene, C₂-C₅alkyne,
      C₁-C₆alkoxy, C₁-C₆haloalkoxy, and C₃-C₈heterocycloalkyl, wherein the C₁-C₆alkyl, aryl, heteroaryl, C₃-C₈cycloalkyl,
      C₁-C₆heteroalkyl, C₁-C₆haloalkyl, C₂-C₅alkene, C₂-C₅alkyne,
      C₁-C₆alkoxy, C₁-C₆haloalkoxy, and C₃-C₈heterocycloalkyl groups of R⁷ are each optionally substituted with 1 to 3 R¹³ groups;
   each R⁸ is independently selected from H, —CH(R¹⁰)₂, C₁-C₈alkyl,
      C₂-C₈alkene, C₂-C₈alkyne, C₁-C₆haloalkyl, C₁-C₆alkoxy,
      C₁-C₆heteroalkyl, C₃-C₈cycloalkyl, C₂-C₈heterocycloalkyl,
      C₁-C₆hydroxyalkyl and C₁-C₆haloalkoxy, wherein the C₁-C₈alkyl, C₂-C₈alkene, C₂-C₈alkyne, C₁-C₆heteroalkyl, C₁-C₆haloalkyl,
      C₁-C₆alkoxy, C₃-C₈cycloalkyl, C₂-C₈heterocycloalkyl,
      C₁-C₆hydroxyalkyl and C₁-C₆haloalkoxy groups of R⁸ are each optionally substituted with 1 to 3 substituents independently selected from —CN, R¹¹,
      —OR¹¹, —SR¹¹, —C(O)R¹¹, —OC(O)R¹¹,
      —C(O)N(R⁹)₂, —C(O)OR¹¹, —NR⁹C(O)R¹¹, —NR⁹R¹⁰, —NR¹¹R¹²,
      —N(R⁹)₂, —OR⁹, —OR¹⁰, —C(O)NR¹¹R¹², —C(O)NR¹¹OH, —S(O)₂R¹¹, —S(O)R¹¹, —S(O)₂NR¹¹R¹², —NR¹¹S(O)₂R¹¹, —P(O)(OR¹)₂, and
      —OP(O)(OR¹¹)₂;
   each R⁹ is independently selected from H, —C(O)R⁸, —C(O)OR⁸, —C(O)R¹⁰, —C(O)OR¹⁰, —S(O)₂R¹⁰, —C₁-C₆ alkyl, C₁-C₆ heteroalkyl and C₃-C₆ cycloalkyl, or each R⁹ is independently a C₁-C₆alkyl that together with N they are attached to form a C₃-C₈heterocycloalkyl, wherein the C₃-C₈heterocycloalkyl ring optionally contains an additional heteroatom selected from N, O and S, and wherein the C₁-C₆ alkyl, C₁-C₆ heteroalkyl,
      C₃-C₆ cycloalkyl, or
      C₃-C₈heterocycloalkyl groups of R⁹ are each optionally substituted with 1 to 3 substituents independently selected from
      —CN, R¹¹, —OR¹¹, —SR¹¹, —C(O)R¹¹, —OC(O)R¹¹, —C(O)OR¹¹,
      —NR¹¹R¹², —C(O)NR¹¹R¹², —C(O)NR¹¹OH, —S(O)₂R¹, —S(O)R¹¹,
      —S(O)₂NR¹¹R¹², —NR¹¹S(O)₂R¹¹, —P(O)(OR¹)₂, and
      —OP(O)(OR¹¹)₂;
   each R¹⁰ is independently selected from aryl, C₃-C₈cycloalkyl,
      C₃-C₈heterocycloalkyl and heteroaryl, wherein the aryl,
      C₃-C₈cycloalkyl, C₃-C₈heterocycloalkyl and heteroaryl groups are optionally substituted with 1 to 3 substituents selected from halogen, —R⁸, —OR⁸, -LR⁹, -LOR⁹, —N(R⁹)₂, —NR⁹C(O)R⁸,
      —NR⁹CO₂R⁸, —CO₂R⁸, —C(O)R⁸ and —C(O)N(R⁹)₂;
   R¹¹ and R¹² are independently selected from H, C₁-C₆alkyl,
      C₁-C₆heteroalkyl, C₁-C₆haloalkyl, aryl, heteroaryl,
      C₃-C₈cycloalkyl, and C₃-C₈heterocycloalkyl, wherein the
      C₁-C₆alkyl, C₁-C₆heteroalkyl, C₁-C₆haloalkyl, aryl, heteroaryl, C₃-C₈cycloalkyl, and C₃-C₈heterocycloalkyl groups of R¹¹ and R¹² are each optionally substituted with 1 to 3 substituents independently selected from halogen, —CN, R⁸, —OR⁸, —C(O)R⁸,
      —OC(O)R⁸, —C(O)OR⁸, —N(R⁹)₂, —NR⁸C(O)R⁸, —NR⁸C(O)OR⁸,
      —C(O)N(R⁹)₂, C₃-C₈heterocycloalkyl, —S(O)₂R⁸, —S(O)₂N(R⁹)₂,
      —NR⁹S(O)₂R⁸, C₁-C₆haloalkyl and C₁-C₆haloalkoxy;
   or R¹¹ and R¹² are each independently C₁-C₆alkyl and taken together with the N atom to which they are attached form an optionally substituted C₃-C₈heterocycloalkyl ring optionally containing an additional heteroatom selected from N, O and S;
   each R¹³ is independently selected from halogen, —CN, -LR⁹, -LOR⁹, —OLR⁹, -LR¹⁰, -LOR¹⁰, -OLR¹⁰, -LR⁸, -LOR⁸, -OLR⁸, -LSR⁸,
      -LSR¹⁰, -LC(O)R⁸, -OLC(O)R⁸, -LC(O)OR⁸, -LC(O)R¹⁰,
      -LOC(O)OR⁸, -LC(O)NR⁹R¹¹, -LC(O)NR⁹R⁸, -LN(R⁹)₂,
      -LNR⁹R⁸, -LNR⁹R¹⁰, -LC(O)N(R⁹)₂, -LS(O)₂R⁸, -LS(O)R⁸,
      -LC(O)NR⁸OH, -LNR⁹C(O)R⁸, -LNR⁹C(O)OR⁸, -LS(O)₂N(R⁹)₂, -OLS(O)₂N(R⁹)₂, -LNR⁹S(O)₂R⁸,
      -LC(O)NR⁹LN(R⁹)₂, -LP(O)(OR⁸)₂, -LOP(O)(OR⁸)₂,
      -LP(O)(OR¹⁰)₂ and -OLP(O)(OR¹⁰)₂;
   each R^{A} is independently selected from —R⁸, —R⁷, —OR⁷, —OR⁸, —R¹⁰,
      —OR¹⁰, —SR⁸, —NO₂, —CN, —N(R⁹)₂, —NR⁹C(O)R⁸, —NR⁹C(S)R⁸,
      —NR⁹C(O)N(R⁹)₂, —NR⁹C(S)N(R⁹)₂, —NR⁹CO₂R⁸,
      —NR⁹NR⁹C(O)R⁸, —NR⁹NR⁹C(O)N(R⁹)₂, —NR⁹NR⁹CO₂R⁸,
      —C(O)C(O)R⁸, —C(O)CH₂C(O)R⁸, —CO₂R⁸, —(CH₂), CO₂R⁸,
      —C(O)R⁸, —C(S)R⁸, —C(O)N(R⁹)₂, —C(S)N(R⁹)₂, —OC(O)N(R⁹)₂,
      —OC(O)R⁸, —C(O)N(OR⁸)R⁸, —C(NOR⁸)R⁸, —S(O)₂R⁸, —S(O)₃R⁸,
      —SO₂N(R⁹)₂, —S(O)R⁸, —NR⁹SO₂N(R⁹)₂, —NR⁹SO₂R⁸, —P(O)(OR⁸)₂, —OP(O)(OR⁸)₂, —P(O)(OR¹⁰)₂, —OP(O)(OR¹⁰)₂, —N(OR⁸)R⁸,
      —CH═CHCO₂R⁸, —C(═NH)—N(R⁹)₂, and —(CH₂). NHC(O)R⁸;
n is, independently at each occurrence, 0, 1, 2, 3, 4, 5, 6, 7 or 8;
each m is independently selected from 1, 2, 3, 4, 5 and 6, and
t is 1, 2, 3, 4, 5, 6, 7 or 8; or (c) a pharmaceutically acceptable salt of any of (a) or (b).

Other benzonaphthyridine compounds, and methods of making benzonaphthyridine compounds, are described in WO 2009/111337. A benzonaphthyridine compound, or a salt thereof, can be used on its own, or in combination with one or more further compounds. For example, a benzonaphthyridine compound can be used in combination with an oil-in-water emulsion or a mineral-containing composition. In a particular embodiment, a benzonaphthyridine compound is used in combination with an oil-in-water emulsion (e.g. a squalene-water emulsion, such as MF59) or a mineral-containing composition (e.g., a mineral sald such as an aluminum salt or a calcium salt).
A thiosemicarbazone compound, such as those disclosed in reference 94. Methods of formulating, manufacturing, and screening for active compounds are also described in reference 94. The thiosemicarbazones are particularly effective in the stimulation of human peripheral blood mononuclear cells for the production of cytokines, such as TNF-α.
A tryptanthrin compound, such as those disclosed in reference 95. Methods of formulating, manufacturing, and screening for active compounds are also described in reference 95. The thiosemicarbazones are particularly effective in the stimulation of human peripheral blood mononuclear cells for the production of cytokines, such as TNF-α.
A nucleoside analog, such as: (a) Isatorabine (ANA-245; 7-thia-8-oxoguanosine):

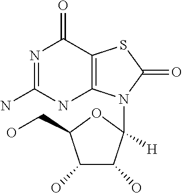 [SMILES: Nc1nc(=O)c2sc(=O)n([C@@H]3O[C@H](CO)[C@@H](O)[C@H]3O)c2[nH]1]

and prodrugs thereof; (b) ANA975; (c) ANA-025-1; (d) ANA380; (e) the compounds disclosed in references 96 to 98; (f) a compound having the formula:

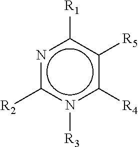

wherein:
   R₁ and R₂ are each independently H, halo, —NRₐR_{b}, —OH, C₁₋₆ alkoxy, substituted C₁₋₆ alkoxy, heterocyclyl, substituted heterocyclyl, C₆₋₁₀ aryl, substituted C₆₋₁₀ aryl, C₁₋₆ alkyl, or substituted C₁₋₆ alkyl;
   R₃ is absent, H, C₁₋₆ alkyl, substituted C₁₋₆ alkyl, C₆₋₁₀ aryl, substituted C₆₋₁₀ aryl, heterocyclyl, or substituted heterocyclyl;
   R₄ and R₅ are each independently H, halo, heterocyclyl, substituted heterocyclyl, —C(O)—R_{d}, C₁₋₆ alkyl, substituted C₁₋₆ alkyl, or bound together to form a 5 membered ring as in R₄₋₅:

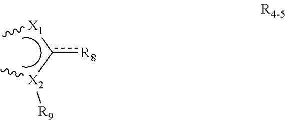

the binding being achieved at the bonds indicated by a [symbol]
X₁ and X₂ are each independently N, C, O, or S;
R₈ is H, halo, —OH, C₁₋₆ alkyl, C₂₋₆ alkenyl, C₂₋₆ alkynyl, —OH, —NRₐR_{b}, —(CH₂)ₙ—O—R_{c}, —O—(C₁₋₆ alkyl), —S(O)ₚRₑ, or —C(O)—R_{d};
R₉ is H, C₁₋₆ alkyl, substituted C₁₋₆ alkyl, heterocyclyl, substituted heterocyclyl or R₉ₐ, wherein R₉ₐ is:

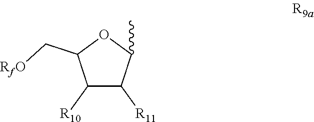

the binding being achieved at the bond indicated by a [symbol] R₁₀ and R₁₁ are each independently H, halo, C₁₋₆ alkoxy, substituted C₁₋₆ alkoxy, —NRₐR_{b}, or —OH;
each Rₐ and R_{b} is independently H, C₁-6 alkyl, substituted C₁₋₆ alkyl, —C(O)R_{d}, C₆-10 aryl;
each R_{c} is independently H, phosphate, diphosphate, triphosphate, C₁₋₆ alkyl, or substituted C₁₋₆ alkyl;
each R_{d} is independently H, halo, C₁₋₆ alkyl, substituted C₁₋₆ alkyl, C₁₋₆ alkoxy, substituted C₁₋₆ alkoxy, —NH₂, —NH(C₁₋₆ alkyl), —NH(substituted C₁₋₆ alkyl), —N(C₁₋₆ alkyl)₂, —N(substituted C₁₋₆ alkyl)₂, C₆₋₁₀ aryl, or heterocyclyl;
each Rₑ is independently H, C₁₋₆ alkyl, substituted C₁₋₆ alkyl, C₆₋₁₀ aryl, substituted C₆₋₁₀ aryl, heterocyclyl, or substituted heterocyclyl;
each R_{f} is independently H, C₁₋₆ alkyl, substituted C₁₋₆ alkyl, —C(O)R_{d}, phosphate, diphosphate, or triphosphate;
each n is independently 0, 1, 2, or 3;
each p is independently 0, 1, or 2; or
or (g) a pharmaceutically acceptable salt of any of (a) to (f), a tautomer of any of (a) to (f), or a pharmaceutically acceptable salt of the tautomer.
Loxoribine (7-allyl-8-oxoguanosine) (99).
Compounds disclosed in reference 100, including: Acylpiperazine compounds, Indoledione compounds, Tetrahydraisoquinoline (THIQ) compounds, Benzocyclodione compounds, Aminoazavinyl compounds, Aminobenzimidazole quinolinone (ABIQ) compounds (101, 102), Hydrapthalamide compounds, Benzophenone compounds, Isoxazole compounds, Sterol compounds, Quinazilinone compounds, Pyrrole compounds (103), Anthraquinone compounds, Quinoxaline compounds, Triazine compounds, Pyrazalopyrimidine compounds, and Benzazole compounds (104).
Compounds disclosed in reference 105.
An aminoalkyl glucosaminide phosphate derivative, such as RC-529 (106, 107).
A phosphazene, such as poly[di(carboxylatophenoxy)phosphazene](“PCPP”) as described, for example, in references 108 and 109.
Small molecule immunopotentiators (SMIPs) such as:
N2-methyl-1-(2-methylpropyl)-1H-imidazo[4,5-c]quinoline-2,4-diamine
N2,N2-dimethyl-1-(2-methylpropyl)-1H-imidazo[4,5-c]quinoline-2,4-diamine
N2-ethyl-N2-methyl-1-(2-methylpropyl)-1H-imidazo[4,5-c]quinoline-2,4-diamine
N2-methyl-1-(2-methylpropyl)-N2-propyl-1H-imidazo[4,5-c]quinoline-2,4-diamine
1-(2-methylpropyl)-N2-propyl-1H-imidazo[4,5-c]quinoline-2,4-diamine
N2-butyl-1-(2-methylpropyl)-1H-imidazo[4,5-c]quinoline-2,4-diamine
N2-butyl-N2-methyl-1-(2-methylpropyl)-1H-imidazo[4,5-c]quinoline-2,4-diamine
N2-methyl-1-(2-methylpropyl)-N2-pentyl-1H-imidazo[4,5-c]quinoline-2,4-diamine
N2-methyl-1-(2-methylpropyl)-N2-prop-2-enyl-1H-imidazo[4,5-c]quinoline-2,4-diamine
1-(2-methylpropyl)-2-[(phenylmethyl)thio]-1H-imidazo[4,5-c]quinolin-4-amine
1-(2-methylpropyl)-2-(propylthio)-1H-imidazo[4,5-c]quinolin-4-amine
2-[[4-amino-1-(2-methylpropyl)-1H-imidazo[4,5-c]quinolin-2-yl](methyl)amino]ethanol
2-[[4-amino-1-(2-methylpropyl)-1H-imidazo[4,5-c]quinolin-2-yl](methyl)amino]ethyl acetate
4-amino-1-(2-methylpropyl)-1,3-dihydro-2H-imidazo[4,5-c]quinolin-2-one
N2-butyl-1-(2-methylpropyl)-N4,N4-bis(phenylmethyl)-1H-imidazo[4,5-c]quinoline-2,4-diamine
N2-butyl-N2-methyl-1-(2-methylpropyl)-N4,N4-bis(phenylmethyl)-1H-imidazo[4,5-c]quinoline-2,4-diamine
N2-methyl-1-(2-methylpropyl)-N4,N4-bis(phenylmethyl)-1H-imidazo[4,5-c]quinoline-2,4-diamine
N2,N2-dimethyl-1-(2-methylpropyl)-N4,N4-bis(phenylmethyl)-1H-imidazo[4,5-c]quinoline-2,4-diamine
1-[4-amino-2-[methyl(propyl)amino]-1H-imidazo[4,5-c]quinolin-1-yl]-2-methylpropan-2-ol
1-[4-amino-2-(propylamino)-1H-imidazo[4,5-c]quinolin-1-yl]-2-methylpropan-2-ol
N4,N4-dibenzyl-1-(2-methoxy-2-methylpropyl)-N2-propyl-1H-imidazo[4,5-c]quinoline-2,4-diamine.

The cytokine-inducing agents for use in the present invention may be modulators and/or agonists of Toll-Like Receptors (TLR). For example, they may be agonists of one or more of the human TLR1, TLR2, TLR3, TLR4, TLR7, TLR8, and/or TLR9 proteins. Preferred agents are agonists of TLR4 (e.g., modified natural lipid As derived from enterobacterial lipopolysaccharides, phospholipid compounds, such as the synthetic phospholipid dimer, E6020), TLR7 (e.g., benzonaphthyridines, imidazoquinolines) and/or TLR9 (e.g., CpG oligonucleotides). These agents are useful for activating innate immunity pathways.

The cytokine-inducing agent can be added to the composition at various stages during its production. For example, it may be within an antigen composition, and this mixture can then be added to an oil-in-water emulsion. As an alternative, it may be within an oil-in-water emulsion, in which case the agent can either be added to the emulsion components before emulsification, or it can be added to the emulsion after emulsification. Similarly, the agent may be coacervated within the emulsion droplets. The location and distribution of the cytokine-inducing agent within the final composition will depend on its hydrophilic/lipophilic properties, e.g., the agent can be located in the aqueous phase, in the oil phase, and/or at the oil-water interface.

The cytokine-inducing agent can be conjugated to a separate agent, such as an antigen (e.g., CRM197). A general review of conjugation techniques for small molecules is provided in ref. 110. As an alternative, the adjuvants may be non-covalently associated with additional agents, such as by way of hydrophobic or ionic interactions.

Preferred cytokine-inducing agents are (a) benzonapthridine compounds; (b) immunostimulatory oligonucleotides and (c) 3dMPL.

Immunostimulatory oligonucleotides can include nucleotide modifications/analogs such as phosphorothioate modifications and can be double-stranded or (except for RNA) single-stranded. References 111, 112, and 113disclose possible analog substitutions, e.g., replacement of guanosine with 2′-deoxy-7-deazaguanosine. The adjuvant effect of CpG oligonucleotides is further discussed in refs. 114 to 119. A CpG sequence may be directed to TLR9, such as the motif GTCGTT or TTCGTT (120). The CpG sequence may be specific for inducing a Th1 immune response, such as a CpG-A ODN (oligodeoxynucleotide), or it may be more specific for inducing a B cell response, such a CpG-B ODN. CpG-A and CpG-B ODNs are discussed in refs. 121-123. Preferably, the CpG is a CpG-A ODN. Preferably, the CpG oligonucleotide is constructed so that the 5′ end is accessible for receptor recognition. Optionally, two CpG oligonucleotide sequences may be attached at their 3′ ends to form “immunomers”. See, for example, references 120 & 124-126. A useful CpG adjuvant is CpG7909, also known as PROMUNE (TM) (Coley Pharmaceutical Group, Inc.).

As an alternative, or in addition, to using CpG sequences, TpG sequences can be used (127). These oligonucleotides may be free from unmethylated CpG motifs.

The immunostimulatory oligonucleotide may be pyrimidine-rich. For example, it may comprise more than one consecutive thymidine nucleotide (e.g., TTTT, as disclosed in ref. 127), and/or it may have a nucleotide composition with >25% thymidine (e.g., >35%, >40%, >50%, >60%, >80%, etc.). For example, it may comprise more than one consecutive cytosine nucleotide (e.g., CCCC, as disclosed in ref. 127), and/or it may have a nucleotide composition with >25% cytosine (e.g., >35%, >40%, >50%, >60%, >80%, etc.). These oligonucleotides may be free from unmethylated CpG motifs.

Immunostimulatory oligonucleotides will typically comprise at least 20 nucleotides. They may comprise fewer than 100 nucleotides.

3dMPL (also known as 3 de-O-acylated monophosphoryl lipid A or 3-O-desacyl-4′-monophosphoryl lipid A) is an adjuvant in which position 3 of the reducing end glucosamine in monophosphoryl lipid A has been de-acylated. 3dMPL has been prepared from a heptoseless mutant of Salmonella minnesota, and is chemically similar to lipid A but lacks an acid-labile phosphoryl group and a base-labile acyl group. It activates cells of the monocyte/macrophage lineage and stimulates release of several cytokines, including IL-1, IL-12, TNF-α and GM-CSF (see also ref. 128). Preparation of 3dMPL was originally described in reference 129.

3dMPL can take the form of a mixture of related molecules, varying by their acylation (e.g., having 3, 4, 5 or 6 acyl chains, which may be of different lengths). The two glucosamine (also known as 2-deoxy-2-amino-glucose) monosaccharides are N-acylated at their 2-position carbons (i.e., at positions 2 and 2′), and there is also O-acylation at the 3′ position. The group attached to carbon 2 has formula —NH—CO—CH₂—CR¹R¹′. The group attached to carbon 2′ has formula —NH—CO—CH₂—CR²R²′. The group attached to carbon 3′ has formula —O—CO—CH₂—CR³R³′. A representative structure is:

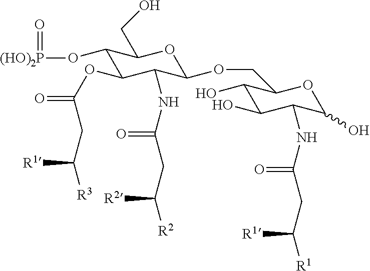

Groups R¹, R² and R³ are each independently —(CH₂)ₙ—CH₃. The value of n is preferably between 8 and 16, more preferably between 9 and 12, and is most preferably 10.

Groups R¹′, R²′ and R³′ can each independently be: (a) —H; (b) —OH; or (c) —O—CO—R⁴, where R⁴ is either —H or —(CH₂)ₘ—CH₃, wherein the value of m is preferably between 8 and 16, and is more preferably 10, 12 or 14. At the 2 position, m is preferably 14. At the 2′ position, m is preferably 10. At the 3′ position, m is preferably 12. Groups R¹′, R²′ and R³′ are thus preferably —O-acyl groups from dodecanoic acid, tetradecanoic acid or hexadecanoic acid.

When all of R¹′, R²′ and R³′ are —H then the 3dMPL has only 3 acyl chains (one on each of positions 2,2′ and 3′). When only two of R¹′, R²′ and R³′ are —H then the 3dMPL can have 4 acyl chains. When only one of R¹′, R²′ and R³′ is —H then the 3dMPL can have 5 acyl chains. When none of R¹, R^{2′} and R^{3′} is —H then the 3dMPL can have 6 acyl chains. The 3dMPL adjuvant used according to the invention can be a mixture of these forms, with from 3 to 6 acyl chains, but it is preferred to include 3dMPL with 6 acyl chains in the mixture, and in particular to ensure that the hexaacyl chain form makes up at least 10% by weight of the total 3dMPL e.g., >20%, >30%, >40%, >50% or more. 3dMPL with 6 acyl chains has been found to be the most adjuvant-active form.

Thus the most preferred form of 3dMPL for inclusion in compositions of the invention has formula (IV), shown below.

Where 3dMPL is used in the form of a mixture then references to amounts or concentrations of 3dMPL in compositions of the invention refer to the combined 3dMPL species in the mixture.

In aqueous conditions, 3dMPL can form micellar aggregates or particles with different sizes e.g., with a diameter <150 nm or >500 nm. Either or both of these can be used with the invention, and the better particles can be selected by routine assay. Smaller particles (e.g., small enough to give a clear aqueous suspension of 3dMPL) are preferred for use according to the invention because of their superior activity (130). Preferred particles have a mean diameter less than 220 nm, more preferably less than 200 nm or less than 150 nm or less than 120 nm, and can even have a mean diameter less than 100 nm. In most cases, however, the mean diameter will not be lower than 50 nm. These particles are small enough to be suitable for filter sterilization. Particle diameter can be assessed by the routine technique of dynamic light scattering, which reveals a mean particle diameter. Where a particle is said to have a diameter of x nm, there will generally be a distribution of particles about this mean, but at least 50% by number (e.g., >60%, >70%, >80%, >90%, or more) of the particles will have a diameter within the range x+25%.

3dMPL can advantageously be used in combination with an oil-in-water emulsion. Substantially all of the 3dMPL may be located in the aqueous phase of the emulsion.

The 3dMPL can be used on its own, or in combination with one or more further compounds. For example, it is known to use 3dMPL in combination with the QS21 saponin (131) (including in an oil-in-water emulsion (132)), with an immunostimulatory oligonucleotide, with both QS21 and an immunostimulatory oligonucleotide, with aluminum phosphate (133), with aluminum hydroxide (134), or with both aluminum phosphate and aluminum hydroxide.

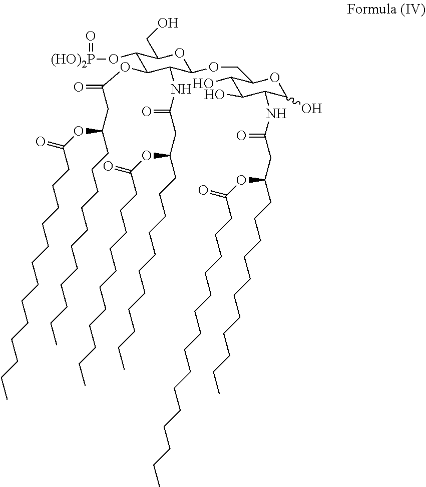

### Fatty Adjuvants

Fatty adjuvants that can be used with the invention include the oil-in-water emulsions described above, and also include, for example:
A phospholipid compound of formula I, II or III, or a salt thereof:

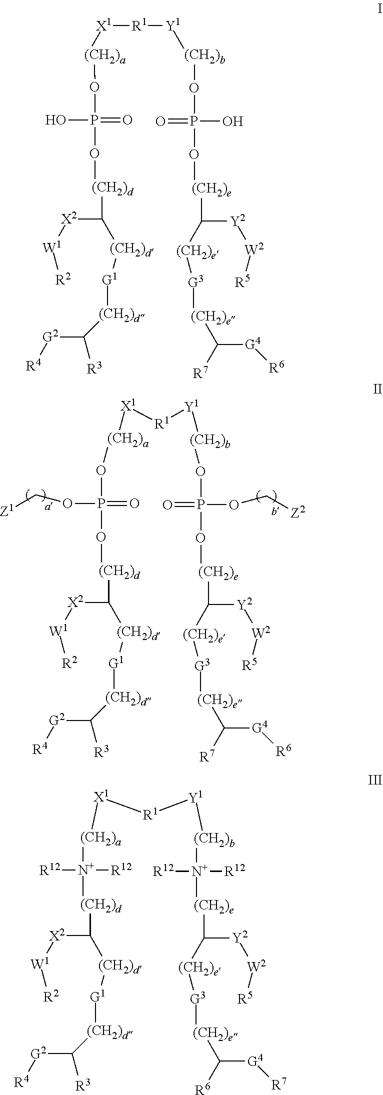

as defined in reference 135, such as ‘ER 803058’, ‘ER 803732’, ‘ER 804053’, ER 804058′, ‘ER 804059’, ‘ER 804442’, ‘ER 804680’, ‘ER 804764’, ER 803022 or ‘ER 804057’ e.g.:

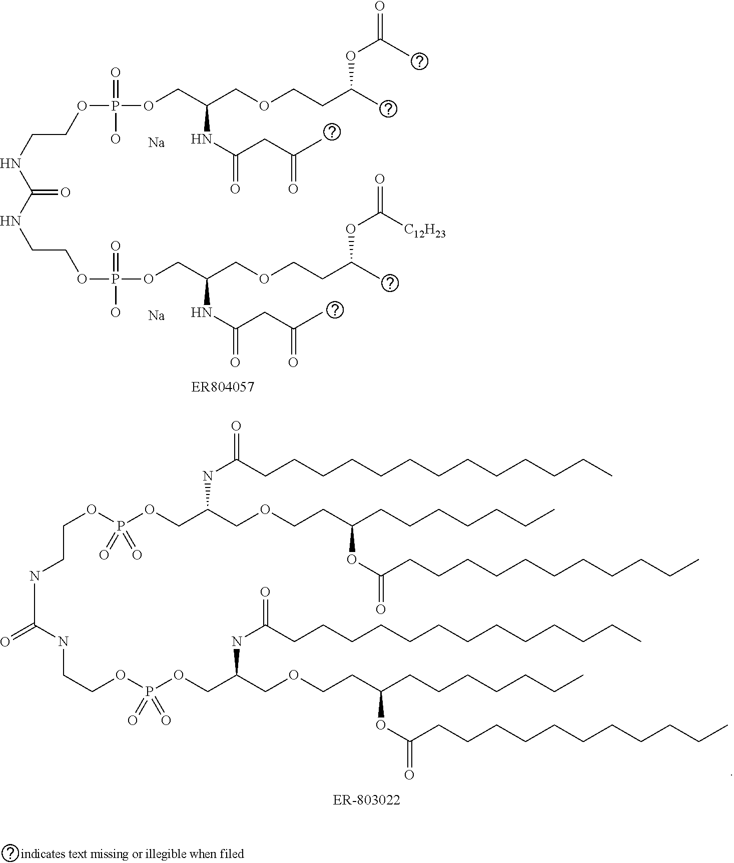

ER804057 is also called E6020. A phospholipid compound of formula I, II or III, or a salt thereof, can be used on its own, or in combination with one or more further compounds. For example, a compound of formula I, II or III, can be used in combination with an oil-in-water emulsion or a mineral-containing composition. In a particular embodiment, E6020 is used in combination with an oil-in-water emulsion (e.g. a squalene-water emulsion, such as MF59) or a mineral-containing composition (e.g., a mineral sald such as an aluminum salt or a calcium salt).
Derivatives of lipid A from Escherichia coli such as OM-174 (described in refs. 136 & 137).
A formulation of a cationic lipid and a (usually neutral) co-lipid, such as aminopropyl-dimethyl-myristoleyloxy-propanaminium bromide-diphytanoylphosphatidyl-ethanolamine (“VAXFECTIN (TM)”) or aminopropyl-dimethyl-bis-dodecyloxy-propanaminium bromide-dioleoylphosphatidylethanolamine (“GAP-DLRIE: DOPE”). Formulations containing (+)—N-(3-aminopropyl)-N,N-dimethyl-2,3-bis(syn-9-tetradeceneyloxy)-1-propanaminium salts are preferred (138).
3-O-deacylated monophosphoryl lipid A (see above).
Compounds containing lipids linked to a phosphate-containing acyclic backbone, such as the TLR4 antagonist E5564 (139, 140):

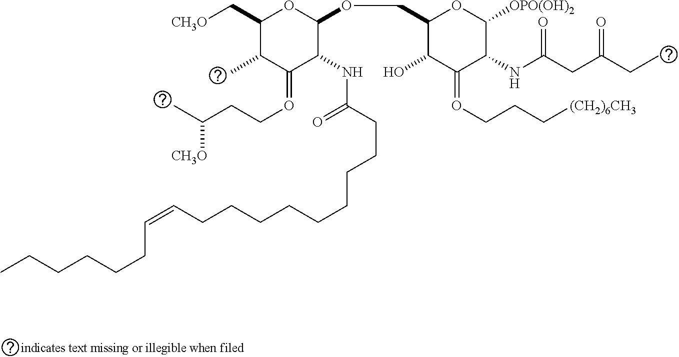

Lipopeptides (i.e., compounds comprising one or more fatty acid residues and two or more amino acid residues), such as lipopeptides based on glycerylcysteine. Specific examples of such peptides include compounds of the following formula

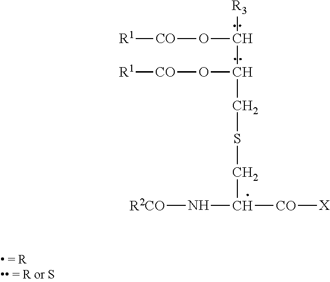

in which each of R¹ and R² represents a saturated or unsaturated, aliphatic or mixed aliphatic-cycloaliphatic hydrocarbon radical having from 8 to 30, preferably 11 to 21, carbon atoms that is optionally also substituted by oxygen functions, R³ represents hydrogen or the radical R₁—CO—O—CH₂— in which R¹ has the same meaning as above, and X represents an amino acid bonded by a peptide linkage and having a free, esterified or amidated carboxy group, or an amino acid sequence of from 2 to 10 amino acids of which the terminal carboxy group is in free, esterified or amidated form. In certain embodiments, the amino acid sequence comprises a D-amino acid, for example, D-glutamic acid (D-Glu) or D-gamma-carboxy-glutamic acid (D-Gla).

Bacterial lipopeptides generally recognize TLR2, without requiring TLR6 to participate. (TLRs operate cooperatively to provide specific recognition of various triggers, and TLR2 plus TLR6 together recognize peptidoglycans, while TLR2 recognizes lipopeptides without TLR6.) These are sometimes classified as natural lipopeptides and synthetic lipopeptides. Synthetic lipopeptides tend to behave similarly, and are primarily recognized by TLR2.

Lipopeptides suitable for use as adjuvants include compounds have the formula:

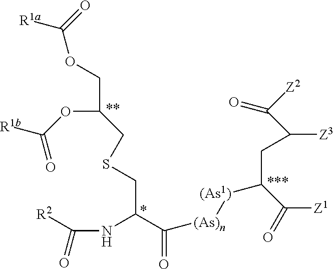

where the chiral center labeled * and the one labeled *** are both in the R configuration; the chiral center labeled ** is either in the R or S configuration;
each R^{1a} and R^{1b} is independently an aliphatic or cycloaliphatic-aliphatic hydrocarbon group having 7-21 carbon atoms, optionally substituted by oxygen functions, or one of R^{1a} and R^{1b}, but not both, is H;
R² is an aliphatic or cycloaliphatic hydrocarbon group having 1-21 carbon atoms and optionally substituted by oxygen functions;
n is 0 or 1;
As represents either —O-Kw-CO— or —NH-Kw-CO—, where Kw is an aliphatic hydrocarbon group having 1-12 carbon atoms;
As¹ is a D- or L-alpha-amino acid;
Z¹ and Z² each independently represent —OH or the N-terminal radical of a D- or L-alpha amino acid of an amino—(lower alkane)—sulfonic acid or of a peptide having up to 6 amino acids selected from the D- and L-alpha aminocarboxylic acids and amino-lower alkyl-sulfonic acids; and
Z³ is H or —CO—Z⁴, wher Z⁴ is —OH or the N-terminal radical of a D- or L-alpha amino acid of an amino-(lower alkane)-sulfonic acid or of a peptide having up to 6 amino acids selected from the D and L-alpha aminocarboxylic acids and amino-lower alkyl-sulfonic acids; or an ester or amide formed from the carboxylic acid of such compounds. Suitable amides include —NH₂ and NH(lower alkyl), and suitable esters include C₁-C₄ alkyl esters. (lower alkyl or lower alkane, as used herein, refers to C₁-C₆ straight chain or branched alkyls).

Such compounds are described in more detail in U.S. Pat. No. 4,666,886. In one particular embodiment, the lipopeptide has the formula:

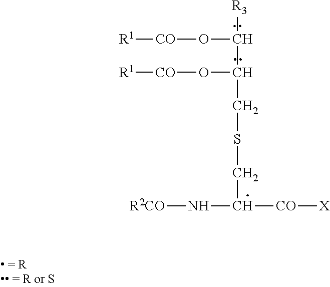

Another example of a lipopeptide species is called LP40, and is an agonist of TLR2. Akdis, et al., Eur. J. Immunology, 33: 2717-26 (2003).

These are related to a known class of lipopeptides from E. coli, referred to as murein lipoproteins. Certain partial degradation products of those proteins called murein lipopetides are described in Hantke, et al., Eur. J. Biochem., 34: 284-296 (1973). These comprise a peptide linked to N-acetyl muramic acid and are thus related to Muramyl peptides, which are described in Baschang, et al., Tetrahedron, 45(20): 6331-6360 (1989).

### Aluminum Salt Adjuvants

The adjuvants known as “aluminum hydroxide” and “aluminum phosphate” may be used. These names are conventional, but are used for convenience only, as neither is a precise description of the actual chemical compound which is present (e.g., see chapter 9 of reference 74). The invention can use any of the “hydroxide” or “phosphate” adjuvants that are in general use as adjuvants.

The adjuvants known as “aluminum hydroxide” are typically aluminum oxyhydroxide salts, which are usually at least partially crystalline. Aluminum oxyhydroxide, which can be represented by the formula AlO(OH), can be distinguished from other aluminum compounds, such as aluminum hydroxide Al(OH)₃, by infrared (IR) spectroscopy, in particular by the presence of an adsorption band at 1070 cm⁻¹ and a strong shoulder at 3090-3100 cm¹ (chapter 9 of ref. 74). The degree of crystallinity of an aluminum hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g., as seen in transmission electron micrographs) is typical for aluminum hydroxide adjuvants. The pI of aluminum hydroxide adjuvants is typically about 11, i.e., the adjuvant itself has a positive surface charge at physiological pH. Adsorptive capacities of between 1.8-2.6 mg protein per mg Al+++ at pH 7.4 have been reported for aluminum hydroxide adjuvants.

The adjuvants known as “aluminum phosphate” are typically aluminum hydroxyphosphates, often also containing a small amount of sulfate (i.e., aluminum hydroxyphosphate sulfate). They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 1.2. Hydroxyphosphates can be distinguished from strict AlPO₄ by the presence of hydroxyl groups. For example, an IR spectrum band at 3164 cm⁻¹ (e.g., when heated to 200° C.) indicates the presence of structural hydroxyls (ch. 9 of ref. 74)

The PO₄/Al³⁺ molar ratio of an aluminum phosphate adjuvant will generally be between 0.3 and 1.2, preferably between 0.8 and 1.2, and more preferably 0.95+0.1. The aluminum phosphate will generally be amorphous, particularly for hydroxyphosphate salts. A typical adjuvant is amorphous aluminum hydroxyphosphate with PO₄/Al molar ratio between 0.84 and 0.92, included at 0.6 mg Al³⁺/ml. The aluminum phosphate will generally be particulate (e.g., plate-like morphology as seen in transmission electron micrographs). Typical diameters of the particles are in the range 0.5-20 μm (e.g., about 5-10 μm) after any antigen adsorption. Adsorptive capacities of between 0.7-1.5 mg protein per mg Al+++ at pH 7.4 have been reported for aluminum phosphate adjuvants.

The point of zero charge (PZC) of aluminum phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminum phosphates used according to the invention will generally have a PZC of between 4.0 and 7.0, more preferably between 5.0 and 6.5, e.g., about 5.7.

Suspensions of aluminum salts used to prepare compositions of the invention may contain a buffer (e.g., a phosphate or a histidine or a Tris buffer), but this is not always necessary. The suspensions are preferably sterile and pyrogen-free. A suspension may include free aqueous phosphate ions e.g., present at a concentration between 1.0 and 20 mM, preferably between 5 and 15 mM, and more preferably about 10 mM. The suspensions may also comprise sodium chloride.

The invention can use a mixture of both an aluminum hydroxide and an aluminum phosphate. In this case there may be more aluminum phosphate than hydroxide e.g., a weight ratio of at least 2:1 e.g., >5:1, >6:1, >7:1, >8:1, >9:1, etc.

The concentration of Al⁺⁺⁺ in a composition for administration to a patient is preferably less than 10 mg/ml e.g., <5 mg/ml, <4 mg/ml, <3 mg/ml, <2 mg/ml, <1 mg/ml, etc. A preferred range is between 0.3 and 1 mg/ml. A maximum of 0.85 mg/dose is preferred.

As well as including one or more aluminum salt adjuvants, the adjuvant component may include one or more further adjuvant or immunostimulating agents. Such additional components include, but are not limited to: a benzonaphthyridine compound, a 3-O-deacylated monophosphoryl lipid A adjuvant (‘3d-MPL’); and/or an oil-in-water emulsion. 3d-MPL has also been referred to as 3 de-O-acylated monophosphoryl lipid A or as 3-O-desacyl-4′-monophosphoryl lipid A. The name indicates that position 3 of the reducing end glucosamine in monophosphoryl lipid A is de-acylated. It has been prepared from a heptoseless mutant of S. minnesota, and is chemically similar to lipid A but lacks an acid-labile phosphoryl group and a base-labile acyl group. It activates cells of the monocyte/macrophage lineage and stimulates release of several cytokines, including IL-1, IL-12, TNF-α and GM-CSF. Preparation of 3d-MPL was originally described in reference 129, and the product has been manufactured and sold by Corixa Corporation under the name MPL (TM). Further details can be found in refs 82 to 85.

The use of an aluminum hydroxide and/or aluminum phosphate adjuvant is useful, particularly in children, and antigens are generally adsorbed to these salts. Squalene-in-water emulsions are also preferred, particularly in the elderly. Useful adjuvant combinations include combinations of Th1 and Th2 adjuvants such as CpG and alum, or resiquimod and alum. A combination of aluminum phosphate and 3dMPL may be used. Other combinations that may be used include: alum and a benzonapthridine compound or a SMIP, a squalene-in-water emulsion (such as MF59) and a benzonapthridine compound or a SMIP, and E6020 and a squalene-in-water emulsion, such as MF59) or alum.

The compositions of the invention may elicit both a cell mediated immune response as well as a humoral immune response.

Two types of T cells, CD4 and CD8 cells, are generally thought necessary to initiate and/or enhance cell mediated immunity and humoral immunity. CD8 T cells can express a CD8 co-receptor and are commonly referred to as Cytotoxic T lymphocytes (CTLs). CD8 T cells are able to recognized or interact with antigens displayed on MHC Class I molecules.

CD4 T cells can express a CD4 co-receptor and are commonly referred to as T helper cells. CD4 T cells are able to recognize antigenic peptides bound to MHC class II molecules. Upon interaction with a MHC class II molecule, the CD4 cells can secrete factors such as cytokines. These secreted cytokines can activate B cells, cytotoxic T cells, macrophages, and other cells that participate in an immune response. Helper T cells or CD4+ cells can be further divided into two functionally distinct subsets: TH1 phenotype and TH2 phenotypes which differ in their cytokine and effector function.

Activated TH1 cells enhance cellular immunity (including an increase in antigen-specific CTL production) and are therefore of particular value in responding to intracellular infections. Activated TH1 cells may secrete one or more of IL-2, IFN-7, and TNF-0. A TH1 immune response may result in local inflammatory reactions by activating macrophages, NK (natural killer) cells, and CD8 cytotoxic T cells (CTLs). A TH1 immune response may also act to expand the immune response by stimulating growth of B and T cells with IL-12. TH1 stimulated B cells may secrete IgG2a.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

An enhanced immune response may include one or more of an enhanced TH1 immune response and a TH2 immune response.

A TH1 immune response may include one or more of an increase in CTLs, an increase in one or more of the cytokines associated with a TH1 immune response (such as IL-2, IFN-γ, and TNF-β), an increase in activated macrophages, an increase in NK activity, or an increase in the production of IgG2a. Preferably, the enhanced TH1 immune response will include an increase in IgG2a production.

A TH1 immune response may be elicited using a TH1 adjuvant. A TH1 adjuvant will generally elicit increased levels of IgG2a production relative to immunization of the antigen without adjuvant. TH1 adjuvants suitable for use in the invention may include for example saponin formulations, virosomes and virus like particles, non-toxic derivatives of enterobacterial lipopolysaccharide (LPS), immunostimulatory oligonucleotides. Immunostimulatory oligonucleotides, such as oligonucleotides containing a CpG motif, are preferred TH1 adjuvants for use in the invention.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune response will include an increase in IgG1 production.

A TH2 immune response may be elicited using a TH2 adjuvant. A TH2 adjuvant will generally elicit increased levels of IgG1 production relative to immunization of the antigen without adjuvant. TH2 adjuvants suitable for use in the invention include, for example, mineral containing compositions, oil-emulsions, and ADP-ribosylating toxins and detoxified derivatives thereof. Mineral containing compositions, such as aluminium salts are preferred TH2 adjuvants for use in the invention.

A composition may include a combination of a TH1 adjuvant and a TH2 adjuvant. Preferably, such a composition elicits an enhanced TH1 and an enhanced TH2 response, i.e., an increase in the production of both IgG1 and IgG2a production relative to immunization without an adjuvant. Still more preferably, the composition comprising a combination of a TH1 and a TH2 adjuvant elicits an increased TH1 and/or an increased TH2 immune response relative to immunization with a single adjuvant (i.e., relative to immunization with a TH1 adjuvant alone or immunization with a TH2 adjuvant alone).

The immune response may be one or both of a TH1 immune response and a TH2 response. Preferably, immune response provides for one or both of an enhanced TH1 response and an enhanced TH2 response.

The enhanced immune response may be one or both of a systemic and a mucosal immune response. Preferably, the immune response provides for one or both of an enhanced systemic and an enhanced mucosal immune response. Preferably the mucosal immune response is a TH2 immune response. Preferably, the mucosal immune response includes an increase in the production of IgA.

### Methods of Treatment, and Administration

Compositions of the invention are suitable for administration to mammals, and the invention provides a method of inducing an immune response in a mammal, comprising the step of administering a composition (e.g., an immunogenic composition) of the invention to the mammal. The compositions (e.g., an immunogenic composition) can be used to produce a vaccine formulation for immunizing a mammal. The mammal is typically a human, and the RSV F protein is typically a human RSV F protein. However, the mammal can be any other mammal that is susceptible to infection with RSV, such as a cow that can be infected with bovine RSV. For example, the immune response may be raised following administration of a purified RSV F protein, an alphavirus particle, or self-replicating RNA.

The invention also provides the use of two or more pre-fusion chimerid proteins based on two or more different non-RSV (e.g., parainfluenza virus, metapneumovirus) F pre-fusion proteins (i.e., PIV5 and NDV) that each have the same RSV F neutralizing epitopes mutated on the protein surface. Thus, inoculation with one chimeric pre-fusion F, and a second inoculation with the second pre-fusion F may prime several antibodies, some to RSV and some to the template protein backbone. The second inoculation may bias boosting of only the shared RSV F neutralizing epitopes.

The invention also provides a composition of the invention for use as a medicament, e.g., for use in immunizing a patient against RSV infection.

The invention also provides the use of a polypeptide as described above in the manufacture of a medicament for raising an immune response in a patient.

The immune response raised by these methods and uses will generally include an antibody response, preferably a protective antibody response. Methods for assessing antibody responses after RSV vaccination are well known in the art.

Compositions of the invention can be administered in a number of suitable ways, such as intramuscular injection (e.g., into the arm or leg), subcutaneous injection, intranasal administration, oral administration, intradermal administration, transcutaneous administration, transdermal administration, and the like. The appropriate route of administration will be dependent upon the age, health and other characteristics of the mammal. A clinician will be able to determine an appropriate route of administration based on these and other factors.

Immunogenic compositions, and vaccine formulations, may be used to treat both children and adults, including pregnant women. Thus a subject may be less than 1 year old, 1-5 years old, 5-15 years old, 15-55 years old, or at least 55 years old. Preferred subjects for receiving the vaccines are the elderly (e.g., >50 years old, >60 years old, and preferably >65 years), the young (e.g., <6 years old, such as 4-6 years old, <5 years old), and pregnant women. The vaccines are not suitable solely for these groups, however, and may be used more generally in a population.

Treatment can be by a single dose schedule or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, etc. Administration of more than one dose (typically two doses) is particularly useful in immunologically naïve patients. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, and the like).

Vaccine formulations produced using a composition of the invention may be administered to patients at substantially the same time as (e.g., during the same medical consultation or visit to a healthcare professional or vaccination centre) other vaccines.

### General

The term “comprising” encompasses “including” as well as “consisting” and “consisting essentially of” e.g., a composition “comprising” X may consist exclusively of X or may include something additional e.g., X+Y.

The word “substantially” does not exclude “completely” e.g., a composition which is “substantially free” from Y may be completely free from Y. Where necessary, the word “substantially” may be omitted from the definition of the invention.

The term “about” in relation to a numerical value x means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE). Overall, it is preferred to culture cells in the total absence of animal-derived materials.

Where a compound is administered to the body as part of a composition then that compound may alternatively be replaced by a suitable prodrug.

Where a cell substrate is used for reassortment or reverse genetics procedures, it is preferably one that has been approved for use in human vaccine production e.g., as in Ph Eur general chapter 5.2.3.

Identity between polypeptide sequences is preferably determined by the Smith-Waterman homology search algorithm as implemented in the MPSRCH program (Oxford Molecular), using an affine gap search with parameters gap open penalty=12 and gap extension penalty=1.

TABLE 2
Phospholipids

| | |
|---|---|
| DDPC | 1,2-Didecanoyl-sn-Glycero-3-phosphatidylcholine |
| DEPA | 1,2-Dierucoyl-sn-Glycero-3-Phosphate |
| DEPC | 1,2-Erucoyl-sn-Glycero-3-phosphatidylcholine |
| DEPE | 1,2-Dierucoyl-sn-Glycero-3-phosphatidylethanolamine |
| DEPG | 1,2-Dierucoyl-sn-Glycero-3[Phosphatidyl-rac-(1-glycerol . . .) |
| DLOPC | 1,2-Linoleoyl-sn-Glycero-3-phosphatidylcholine |
| DLPA | 1,2-Dilauroyl-sn-Glycero-3-Phosphate |
| DLPC | 1,2-Dilauroyl-sn-Glycero-3-phosphatidylcholine |
| DLPE | 1,2-Dilauroyl-sn-Glycero-3-phosphatidylethanolamine |
| DLPG | 1,2-Dilauroyl-sn-Glycero-3[Phosphatidyl-rac-(1-glycerol . . .) |
| DLPS | 1,2-Dilauroyl-sn-Glycero-3-phosphatidylserine |
| DMG | 1,2-Dimyristoyl-sn-glycero-3-phosphoethanolamine |
| DMPA | 1,2-Dimyristoyl-sn-Glycero-3-Phosphate |
| DMPC | 1,2-Dimyristoyl-sn-Glycero-3-phosphatidylcholine |
| DMPE | 1,2-Dimyristoyl-sn-Glycero-3-phosphatidylethanolamine |
| DMPG | 1,2-Myristoyl-sn-Glycero-3[Phosphatidyl-rac-(1-glycerol . . .) |
| DMPS | 1,2-Dimyristoyl-sn-Glycero-3-phosphatidylserine |
| DOPA | 1,2-Dioleoyl-sn-Glycero-3-Phosphate |
| DOPC | 1,2-Dioleoyl-sn-Glycero-3-phosphatidylcholine |
| DOPE | 1,2-Dioleoyl-sn-Glycero-3-phosphatidylethanolamine |
| DOPG | 1,2-Dioleoyl-sn-Glycero-3[Phosphatidyl-rac-(1-glycerol . . .) |
| DOPS | 1,2-Dioleoyl-sn-Glycero-3-phosphatidylserine |
| DPPA | 1,2-Dipalmitoyl-sn-Glycero-3-Phosphate |
| DPPC | 1,2-Dipalmitoyl-sn-Glycero-3-phosphatidylcholine |
| DPPE | 1,2-Dipalmitoyl-sn-Glycero-3-phosphatidylethanolamine |
| DPPG | 1,2-Dipalmitoyl-sn-Glycero-3[Phosphatidyl-rac-(1-glycerol . . .) |
| DPPS | 1,2-Dipalmitoyl-sn-Glycero-3-phosphatidylserine |
| DPyPE | 1,2-diphytanoyl-sn-glycero-3-phosphoethanolamine |
| DSPA | 1,2-Distearoyl-sn-Glycero-3-Phosphate |
| DSPC | 1,2-Distearoyl-sn-Glycero-3-phosphatidylcholine |
| DSPE | 1,2-Diostearpyl-sn-Glycero-3-phosphatidylethanolamine |
| DSPG | 1,2-Distearoyl-sn-Glycero-3[Phosphatidyl-rac-(1-glycerol . . .) |
| DSPS | 1,2-Distearoyl-sn-Glycero-3-phosphatidylserine |
| EPC | Egg-PC |
| HEPC | Hydrogenated Egg PC |
| HSPC | High purity Hydrogenated Soy PC |
| HSPC | Hydrogenated Soy PC |
| LYSOPC MYRISTIC | 1-Myristoyl-sn-Glycero-3-phosphatidylcholine |
| LYSOPC PALMITIC | 1-Palmitoyl-sn-Glycero-3-phosphatidylcholine |
| LYSOPC STEARIC | 1-Stearoyl-sn-Glycero-3-phosphatidylcholine |
| Milk Sphingomyelin MPPC | 1-Myristoyl,2-palmitoyl-sn-Glycero 3-phosphatidylcholine |
| MSPC | 1-Myristoyl,2-stearoyl-sn-Glycero-3-phosphatidylcholine |
| PMPC | 1-Palmitoyl,2-myristoyl-sn-Glycero-3-phosphatidylcholine |
| POPC | 1-Palmitoyl,2-oleoyl-sn-Glycero-3-phosphatidylcholine |
| POPE | 1-Palmitoyl-2-oleoyl-sn-Glycero-3-phosphatidylethanolamine |
| POPG | 1,2-Dioleoyl-sn-Glycero-3[Phosphatidyl-rac-(1-glycerol) . . .] |
| PSPC | 1-Palmitoyl,2-stearoyl-sn-Glycero-3-phosphatidylcholine |
| SMPC | 1-Stearoyl,2-myristoyl-sn-Glycero-3-phosphatidylcholine |
| SOPC | 1-Stearoyl,2-oleoyl-sn-Glycero-3-phosphatidylcholine |
| SPPC | 1-Stearoyl,2-palmitoyl-sn-Glycero-3-phosphatidylcholine |

### EXEMPLIFICATION

### 1. Post-Fusion Structure and Pre-Fusion Model

### Post-Fusion RSV F Structure

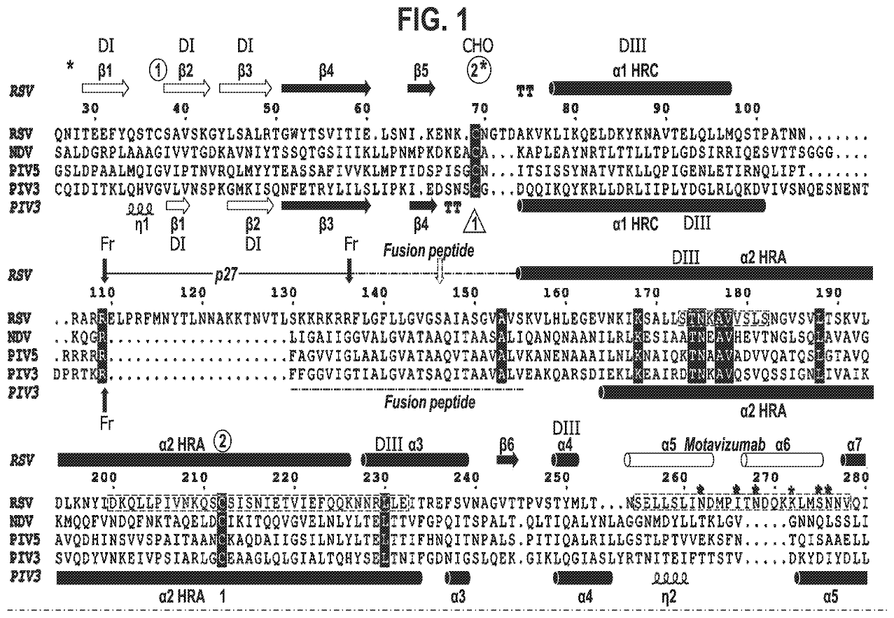

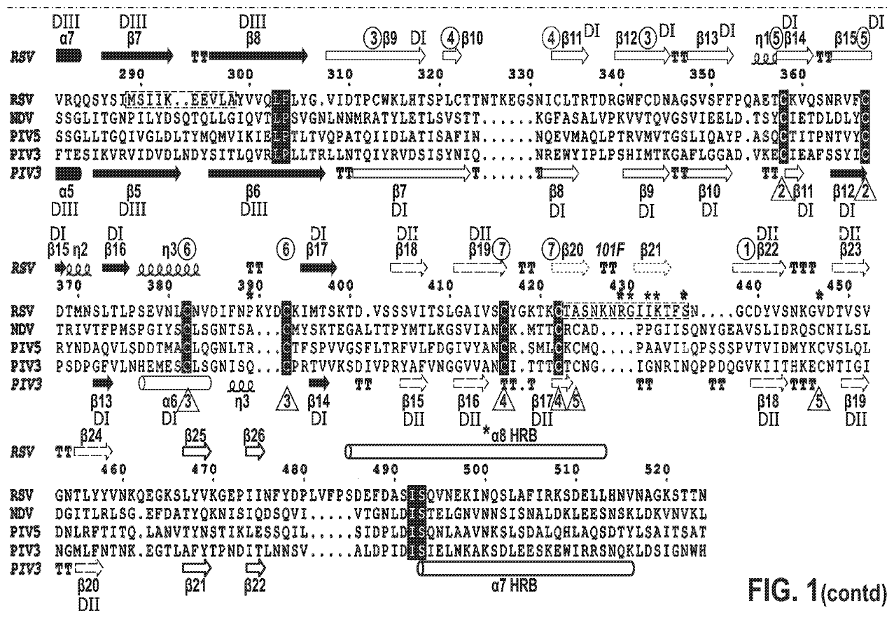

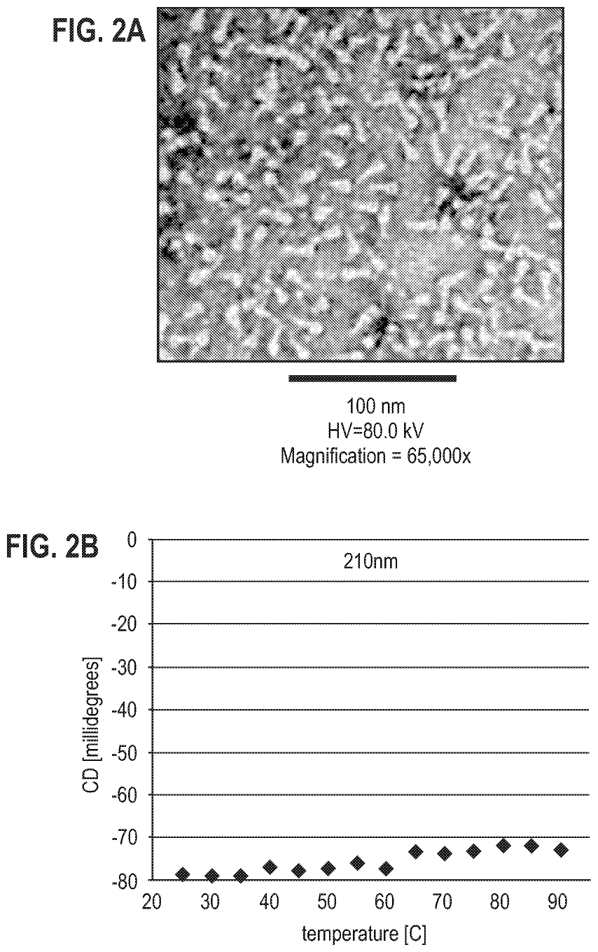

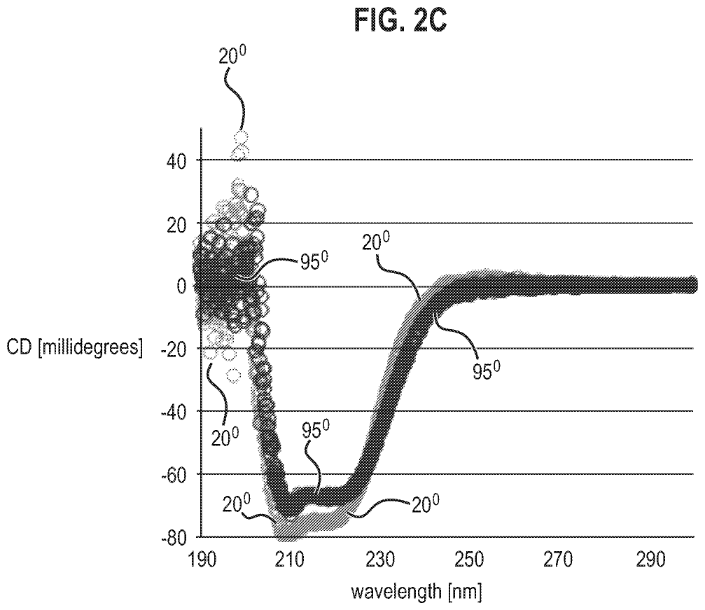

A stable, non-aggregating soluble RSV F subunit antigen was prepared by deletion of the fusion peptide, transmembrane region, and cytoplasmic domain (FIG. 1). This engineered F was expressed efficiently and was readily purified. Electron microscopy of negatively stained specimens showed that it formed non-aggregated, homogeneous crutch-shaped molecules, consistent with post-fusion F trimers (FIG. 2A). This engineered F trimer was stable, and circular dichroism spectroscopy revealed no significant melting at temperatures up to 95° C. (FIGS. 2B and 2C).

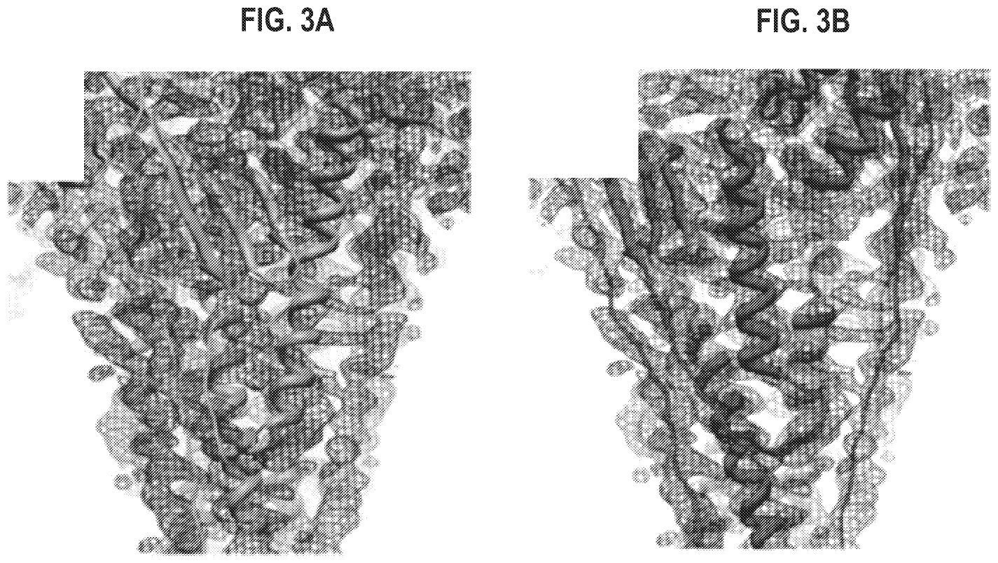

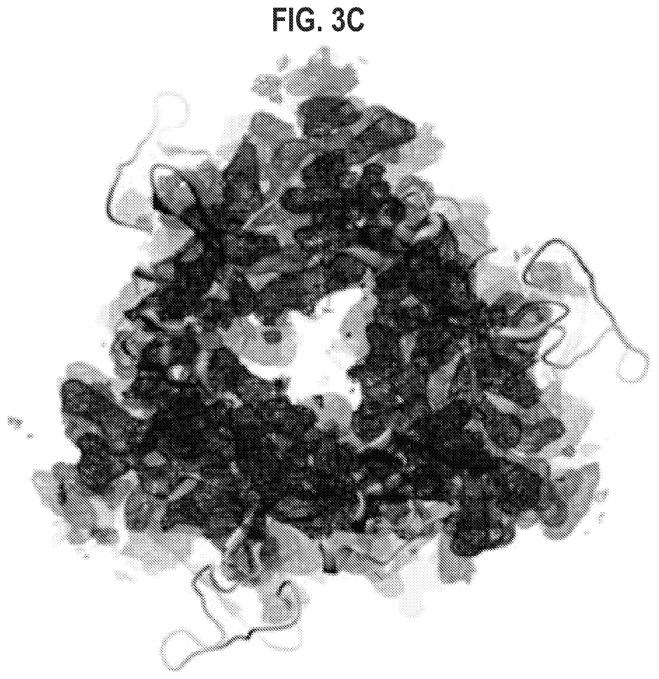

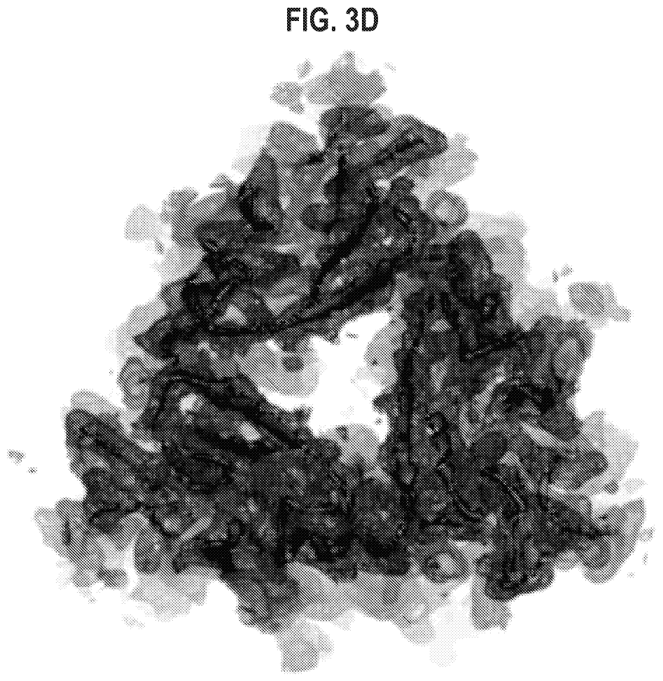

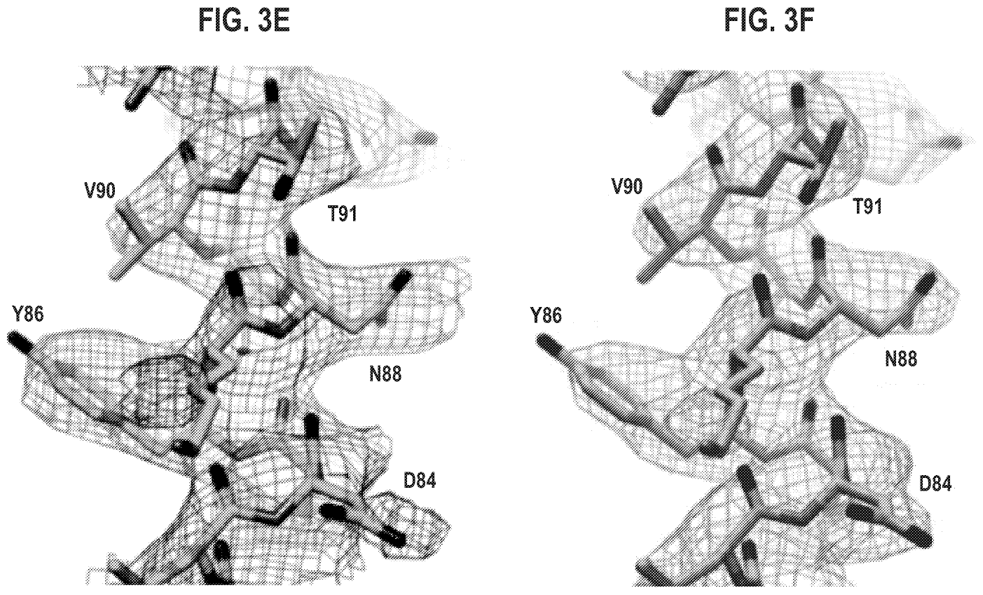

This RSV F protein was crystallized and its structure was determined by molecular replacement and three-fold non-crystallographic symmetry (NCS) averaging (Table 3 and FIG. 3). The structure does not include the p27 fragment (the peptide between the two furin sites that is lost upon cleavage), the fusion peptide, the transmembrane region, or the cytoplasmic domain (FIG. 4).

TABLE 3
Crystallographic data

| Data collection statistics | | |
|---|---|---|
| Space Group | P 2₁ 2₁ 2₁ | |
| Cell dimensions (Å) | a = 87.930 | |
| | b = 113.160 | |
| | c = 311.370 | |
| | α = β = γ = 90.00° | |
| Resolution limit (Å) | 50-3.2 | |
| Unique reflections | 51,911 | |
| Unique reflections^{†} | 40,398 | |
| Redundancy | 3.9 | (3.7)^{¥} |
| Overall completeness (%) | 99.4 | (99.4) |
| Overall completeness (%)^{†} | 77.0 | (26.7) |
| <I/σ> | 12.2 | (2.2) |
| R_{sym} (%) | 7.7 | (71.0) |
| Refinement Statistics^{††} | | |
| Polypeptide chains | 3 | |
| Protein atoms | 10,398 | |
| Residues in allowed regions of | 98.5 | |
| the Ramachandran plot (%) | | |
| Residues in most favored regions | 83.5 | |
| of Ramachandran plot (%) | | |
| RMSD bond lengths (Å) | 0.021 | |
| RMSD bond angles (deg) | 2.053 | |
| Mean B values (Å²) | 15.71 | |
| Resolution range (Å) | 30-3.2 | |
| R_{work} (%) | 23.1 | (34.9) |
| R_{free} (%) | 26.6 | (40.2) |

^{¥}Values in parentheses refer to data in the highest resolution shell
^{†}Statistics for the data after anisotropic correction.
^{††}Refinement values for the data after anisotropic correction.

The overall architecture of post-fusion RSV F is shared with other post-fusion parainfluenza fusion proteins. The protein is composed of three tightly intertwined subunits, forming a globular head and an elongated stalk. Each subunit contains three domains, designated I, II, and III (FIGS. 4A-C). Domains I and II are at the top of the trimeric head and form a triangular crown. Domain III forms the base of the head. A long helix, HRA, extends from domain III and forms the trimeric coiled-coil in the center of the stalk. The HRB helix is tethered to domain II and reaches down to the head-distal end of the stalk, where it forms the outer coils of a six-helix bundle with the HRA interior coiled-coil. In full-length F, the hydrophobic fusion peptide (N-terminal to HRA) and the transmembrane region (C-terminal to HRB), would be juxtaposed at the bottom of the stalk and inserted into the target cell membrane.

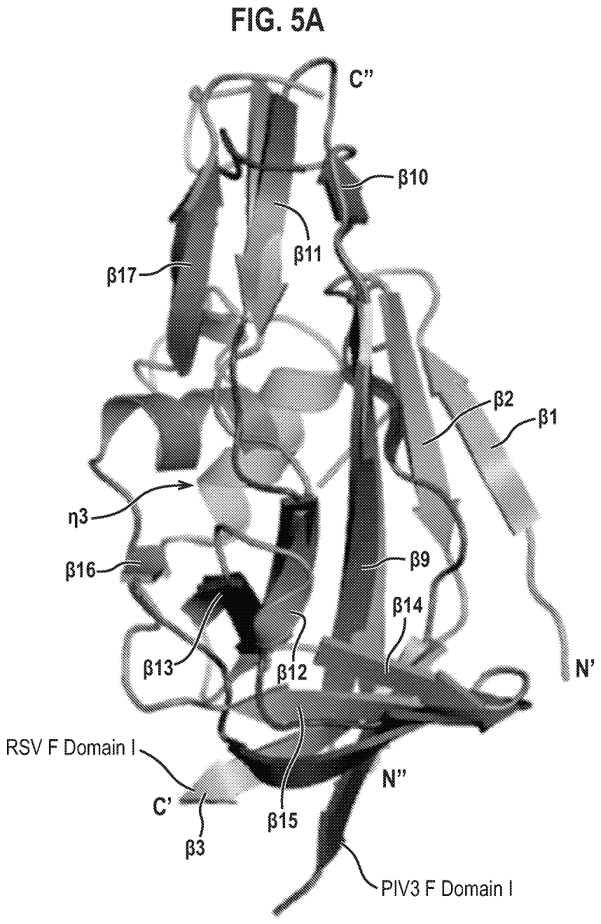

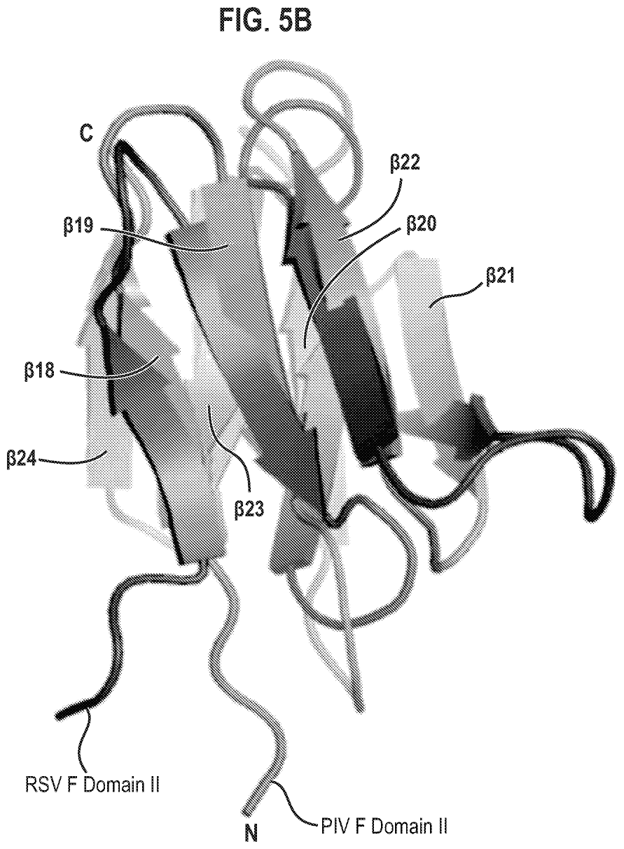

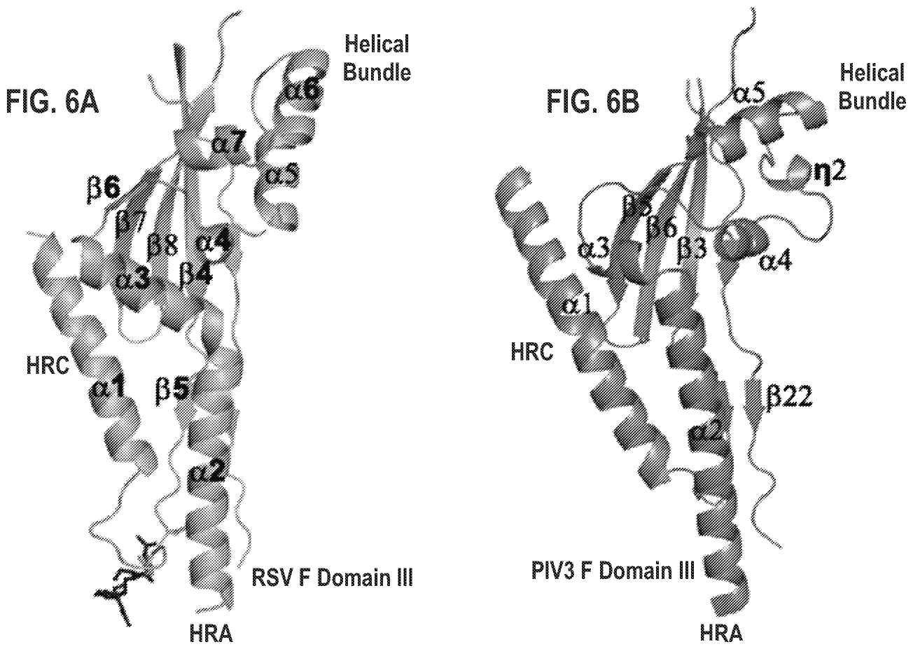

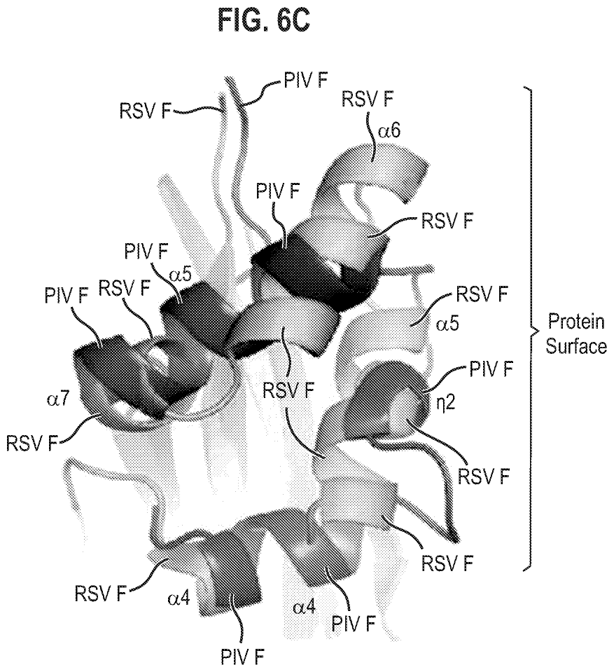

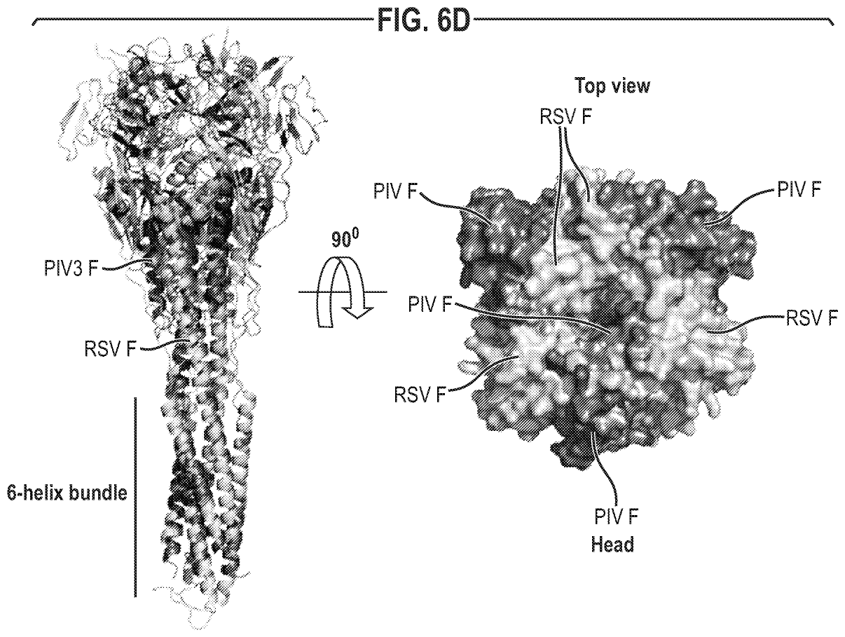

Domains I and II of RSV, parainfluenza virus 3 (PIV3), and parainfluenza virus 5 (PIV5) F proteins are structurally conserved (FIGS. 5A and 5B). The only significant difference is in the orientation of the sole helix of domain I (η3 of RSV F and a6 of PIV3 and PIV5 Fs) relative to their common β-sheets. In contrast, RSV F domain III has features that were not predicted from parainfluenza-based modeling (FIG. 6). When the four-stranded β-sheets of RSV F domain III and PIV3 F domain III are superimposed, key differences in the domains' helical regions are apparent. Helix HRA kinks at a more N-terminal position in RSV F than in PIV3 F, causing an approximately 60° difference in the rotation of the heads relative to the stalks (FIGS. 6A, 6B, 6D). Influenza hemagglutinins also vary in the orientations of their heads relative to their stalks, with 30°-50° differences in rotation between subtypes. (Ha, Y. et al., Embo Journal 21, 865-875 (2002)).

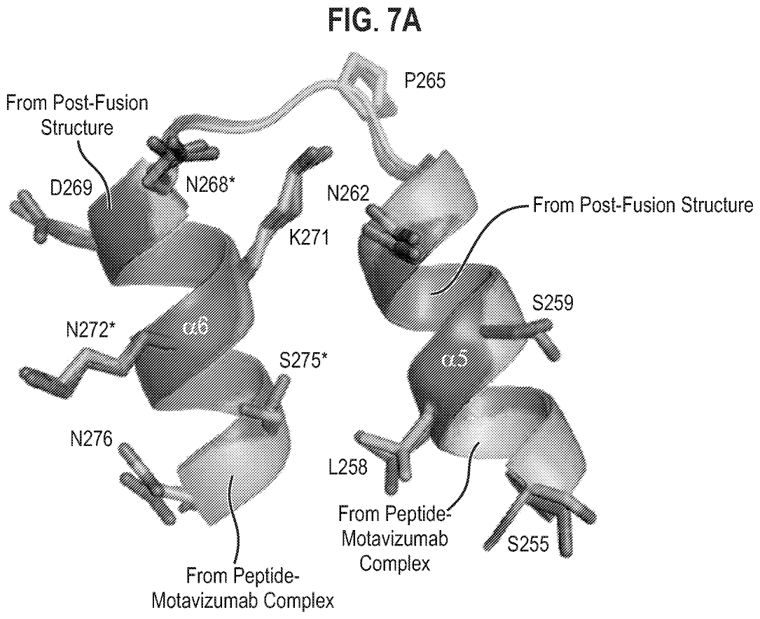

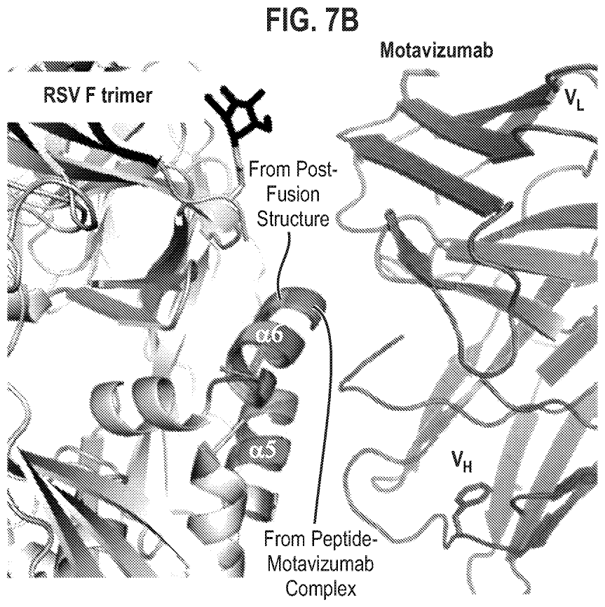

The RSV F domain III helical bundle region contains an extra helix (a6), changing the orientation of the bundled helices relative to those in parainfluenza Fs (FIGS. 6A-C and FIG. 1). RSV F helices α5 and α6 are almost parallel and are exposed on the trimer surface; the equivalent to RSV F α6 helix in the PIV3 helical bundle (a5, FIG. 6C) is buried in the inter-subunit interface of the trimer. RSV F helices α5 and α6 form the epitope bound by the related neutralizing antibodies Palivizumab and Motavizumab. The structure of a complex between the Motavizumab Fab and a 24-residue RSV F peptide that includes a5 and a6 has been reported (McLellan, J. S. et al. Nat Struct Mol Biol 17, 248-250 (2010)). Comparison between this structure and the post-fusion RSV F structure revealed a close match between the α5-α6 helices (r.m.s.d.=0.52 Å; FIG. 7A). Superposition of the two structures based on these helices models a complex between Motavizumab and post-fusion RSV F (FIG. 7B). This model reveals no steric hindrance that would prevent Motavizumab (or, presumably, Palivizumab) from binding to post-fusion RSV F. Binding of Palivizumab to the post-fusion F ectodomain in solution using surface plasmon resonance (K_{D} of 4.2×10⁻¹⁰ M) was confirmed.

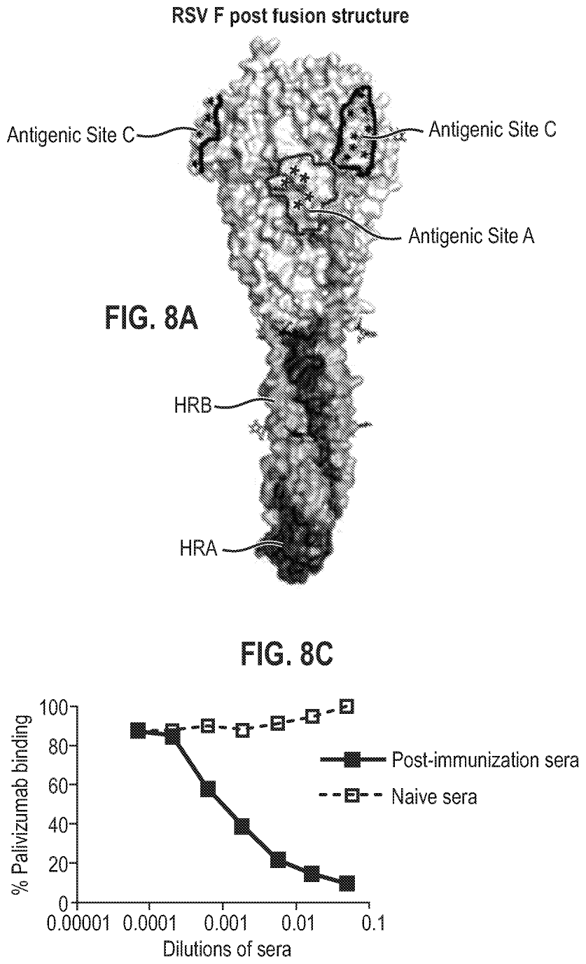

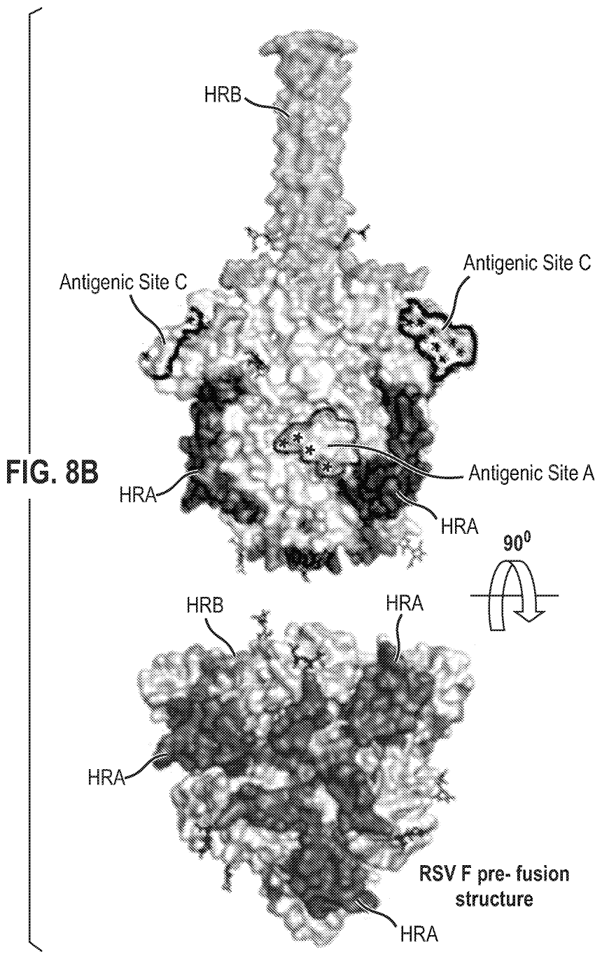

Pre-fusion and post-fusion parainfluenza F structures reveal en bloc shifts of domains and large rearrangements of HRA and HRB. In domain III of the pre-fusion PIV5 F structure (the only reported pre-fusion parainfluenza F structure), HRA folds into three α-helices and two β-strands rather than the long post-fusion HRA helix (Yin, H. S., et al. Nature 439, 38-44 (2006)). However, when pre-fusion and post-fusion conformations of individual parainfluenza F protein domains are compared, the non-rearranging parts superimpose well. Superimposing post-fusion RSV F domains on their pre-fusion PIV5 F counterparts did not result in major clashes and positions all the pairs of cysteines that form interdomain disulfide bonds in proximity (FIG. 8B).

The resulting pre-fusion RSV F model reveals a feature not apparent from modeling pre-fusion RSV F domains based on the PIV5 pre-fusion domain structures (McLellan, J. S. et al. Nat Struct Mol Biol 17, 248-250 (2010)). The helices of the Motavizumab epitope are exposed on the surface of the pre-fusion RSV F trimer, as they are on post-fusion RSV F trimer (FIG. 9). In the current pre-fusion RSV F model, the loop connecting P4 and HRC (part of domain III) would hinder access of Palivizumab or Motavizumab to the epitope. However, the loop may have sufficient flexibility to adopt an alternative conformation that permits antibody binding (FIG. 9B).

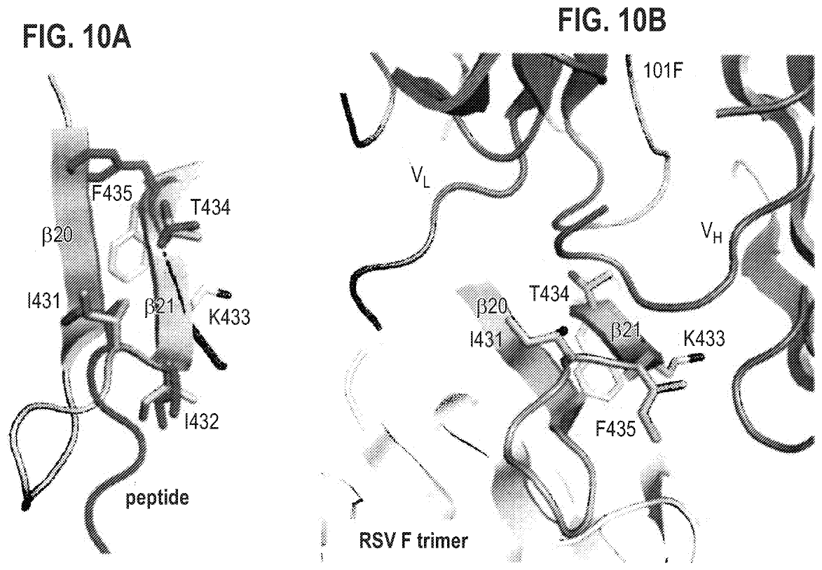

The antigenic structure of RSV F has been mapped by a variety of techniques (FIG. 1). The best documented epitope clusters are designated A and C (Beeler, J. A. & van Wyke Coelingh, K. J Virol 63, 2941-2950 (1989)), and others have been proposed. The Motavizumab-peptide structure corroborated the location of site A, although it called into question the site's exposure on the RSF F trimer (McLellan, J. S. et al. Structural basis of respiratory syncytial virus neutralization by motavizumab. Nat Struct Mol Biol 17, 248-250 (2010)); a crystal structure of an RSV F peptide (residues 422-436) bound to the 101F neutralizing antibody corroborated the location of site C (McLellan, J. S. et al. J Virol 84, 12236-12244 (2010)). The post-fusion structure of RSV F and the pre-fusion RSV F model indicate that sites A and C remain exposed and structurally similar in both conformations (FIGS. 8A and 8B). Superposition of the 101F-peptide complex on the RSV F pre-fusion model and post-fusion structure confirmed that 101F would not clash with F in either conformation (FIG. 10).

Provided in the Appendix is the PDB file of the RSV F pre-fusion model based on the RSV F post-fusion structure and sequence/domain alignments to the PIV5 pre-fusion structure. The PDB file contains the atomic coordinates for the pre-fusion model, and can be used with suitable software for molecular visualization and analysis (e.g., Roger Sayle and E. James Milner-White. “RasMol: Biomolecular graphics for all”, Trends in Biochemical Sciences (TIBS), September 1995, Vol. 20, No. 9, p. 374.) to display the model. Included in the model are the three subunit chains with the fusion peptide and HRA region folded as in PIV5, making significant contacts with DIII. HRB regions of the three subunits trimerize into the pre-fusion stalk and are associated with D1 and DII.

### 2. Destablizing the Post-Fusion 6-Helix Bundle Through Deletion of the HRB Helix

An HRB deletion construct was designed to prevent formation of the post-fusion conformation. Two constructs have been designed to address this strategy. The first is a wild-type ectodomain lacking the HRB region (RSV F residues 1-483) call Del HRB:

The sequence presented below contain a signal peptide and a HIS tag (GGSAGSGHHHHHH; SEQ ID NO:3). The pre-fusion RSV F protein of the invention can contain the amino acid sequences shown below, with or without the signal peptide and/or HIS tag.

>RSV F deIHRB HIS
(SEQ ID NO: 28)
MELLILKANAITTILTAVTFCFASGQNITEEFYQS
TCSAVSKGYLSALRTGWYTSVITIELSNIKENKCN
GTDAKVKLIKQELDKYKNAVTELQLLMQSTPATNN
RARRELPRFMNYTLNNAKKTNVTLSKKRKRRFLGF
LLGVGSAIASGVAVSKVLHLEGEVNKIKSALLSTN
KAVVSLSNGVSVLTSKVLDLKNYIDKQLLPIVNKQ
SCSISNIETVIEFQQKNNRLLEITREFSVNAGVTT
PVSTYMLTNSELLSLINDMPITNDQKKLMSNNVQI
VRQQSYSIMSIIKEEVLAYVVQLPLYGVIDTPCWK
LHTSPLCTTNTKEGSNICLTRTDRGWYCDNAGSVS
FFPQAETCKVQSNRVFCDTMNSLTLPSEVNLCNVD
IFNPKYDCKIMTSKTDVSSSVITSLGAIVSCYGKT
KCTASNKNRGIIKTFSNGCDYVSNKGVDTVSVGNT
LYYVNKQEGKSLYVKGEPIINFYDPLVFPSGGSAG
SGHHHHHH

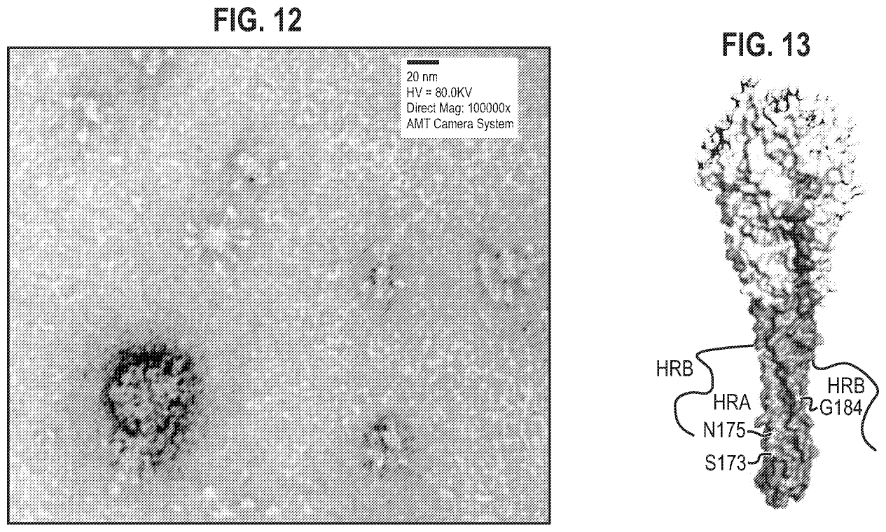

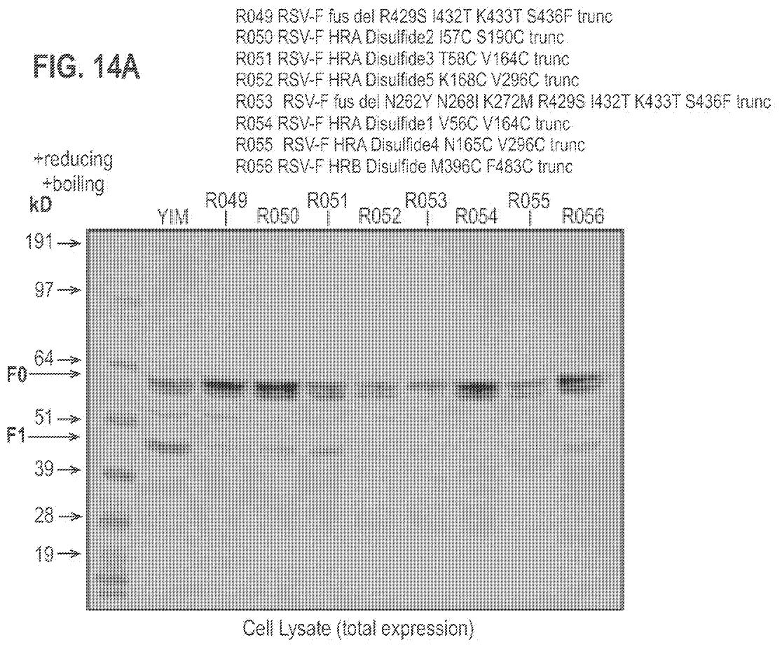

The above sequence has both furin cleavage sites unaltered, and was expected to be processed by the cell. In addition, the above sequence has the wild-type fusion peptide sequence. In previous experiments, when the RSV F ectodomain-based proteins were cleaved by the cell and contained fusion peptides, they formed soluble aggregates with cellular debris in the form of RSV F rosettes. If this construct remained in the pre-fusion conformation (due to the lack of the HRB helix thought to be required for post-fusion conformation) than the fusion peptide should be burried in the RSV F head domain and should not form rosette aggreagates. This construct was expressed, purified by affinity purification and evaluated by EM analysis (FIG. 12).

It is clear both by its migration on an SEC column in the void volume, as well as from the EM micrograph that the construct formed rosettes similar to the rosettes formed by post-fusion RSV F proteins. This result was a surprise as it was hypothesized that the HRB is required to stabilize the HRA in its elongated helix formation (as it is observed that HRA peptides do not form trimers). Thus, we hypothesized that the fusion peptides, binding with one another or with cellular debris, are stabilizing the HRA helixes in their elongated, post-fusion formation.

We have hypothesized that the post-fusion like phenotype of the DelHRB construct was due to stabilization of the HRA into elongated helices by binding of the fusion peptides to one another or cellular debris. To test this hypothesis we are generating the following construct (DelHRB fusion peptide deletion:below) which is similar to the DelHRB but has the fusion peptide deletion consistent with our post-fusion trimer. We will test by EM microscopy the phenotype of the contruct to see if it forms crutch-like structures similar to that of the post-fusion like phenotype observed in the DelHRB rosettes of if the construct forms pre-fusion head shapes, which are similar to the spherical shape of the lollipop phenotype published in the literature.

The sequence presented below contain a signal peptide and a HIS tag (GGSAGSGHHHHHH; SEQ ID NO:3). The pre-fusion RSV F protein of the invention can contain the amino acid sequences shown below, with or without the signal peptide and/or HIS tag.

>RSV F deIHRB fusion peptide
deletion HIS
(SEQ ID NO: 10)
MELLILKANAITTILTAVTFCFASGQNITEEFYQS
TCSAVSKGYLSALRTGWYTSVITIELSNIKENKCN
GTDAKVKLIKQELDKYKNAVTELQLLMQSTPATNN
RARRELPRFMNYTLNNAKKTNVTLSKKRKRRSAIA
SGVAVSKVLHLEGEVNKIKSALLSTNKAVVSLSNG
VSVLTSKVLDLKNYIDKQLLPIVNKQSCSISNIET
VIEFQQKNNRLLEITREFSVNAGVTTPVSTYMLTN
SELLSLINDMPITNDQKKLMSNNVQIVRQQSYSIM
SIIKEEVLAYVVQLPLYGVIDTPCWKLHTSPLCTT
NTKEGSNICLTRTDRGWYCDNAGSVSFFPQAETCK
VQSNRVFCDTMNSLTLPSEVNLCNVDIFNPKYDCK
IMTSKTDVSSSVITSLGAIVSCYGKTKCTASNKNR
GIIKTFSNGCDYVSNKGVDTVSVGNTLYYVNKQEG
KSLYVKGEPIINFYDPLVFPSGGSAGSGHHHHHH

3. Prefusion Stabilization with Intrachain Disulfide Bond Formation

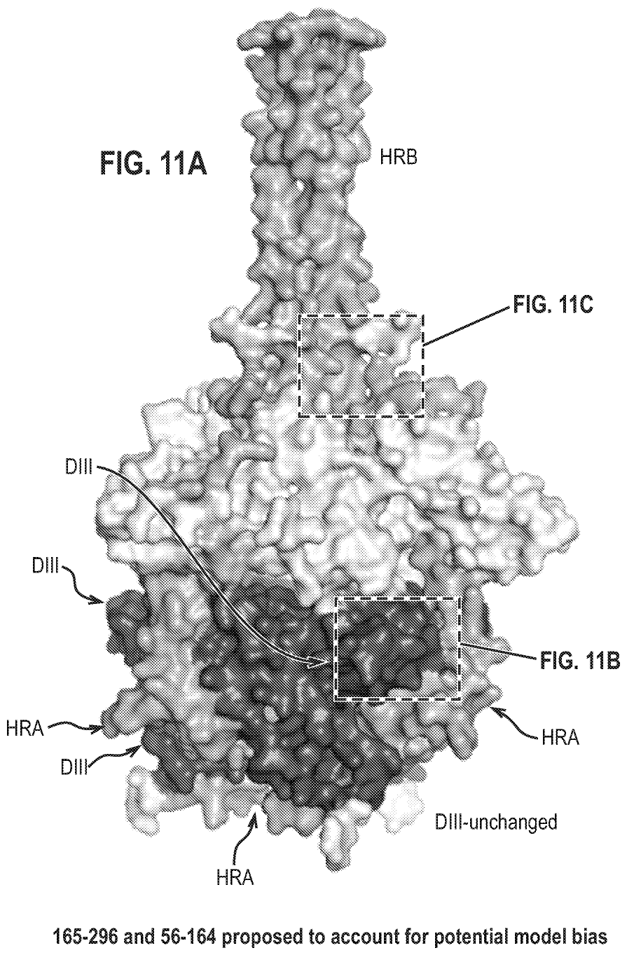

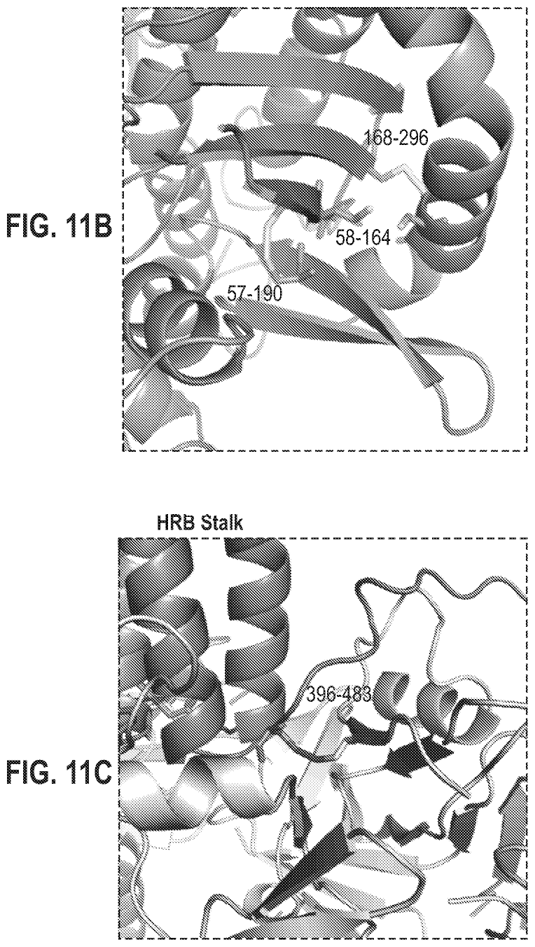

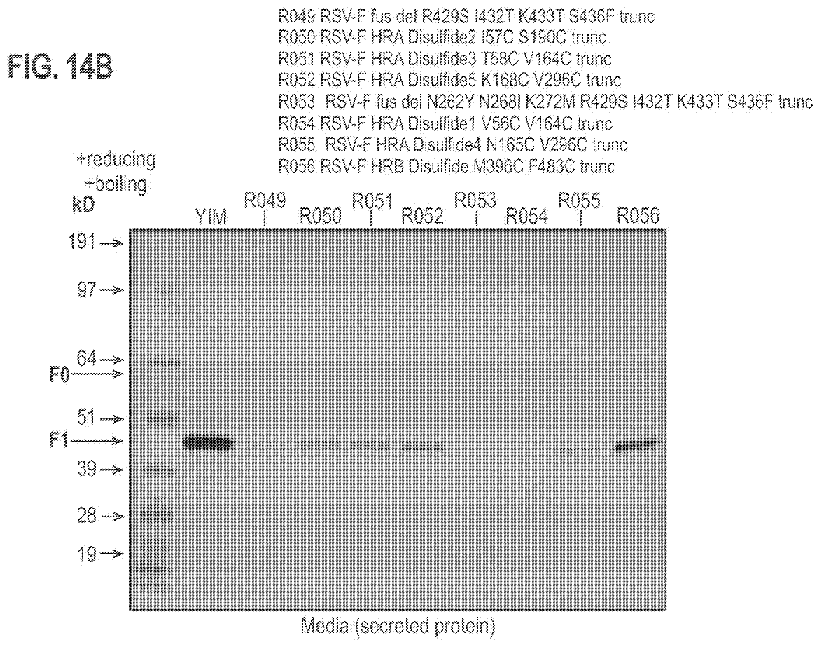

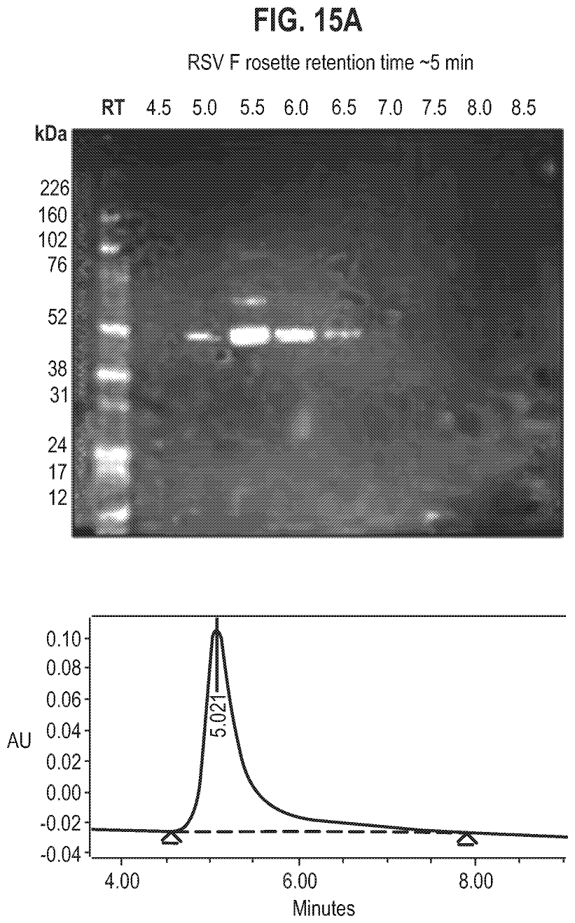

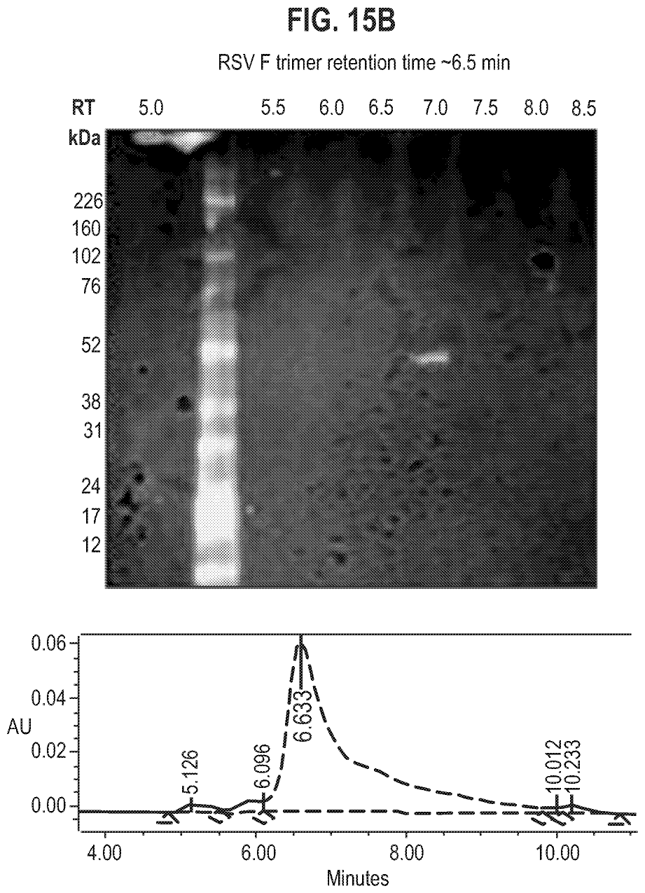

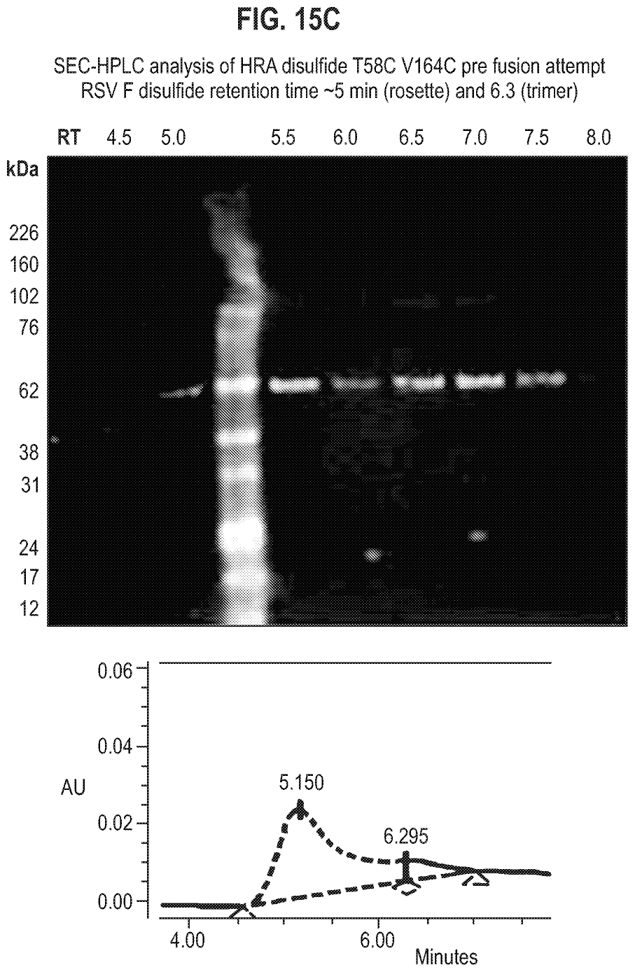

The RSV F model, based on the RSV F post-fusion structure and PIV5 pre-fusion structure, was used to engineer cysteine mutations intended to form disulfide bonds that stabilize RSV F in the pre-fusion conformation (FIG. 11). The intrachain disulfide bond constructs were expressed, and were secreted from the cell into the media and then cleaved from F0 to F1/F2 to various degrees (FIG. 14). RSV F T58C/V164C (expressed in mammalian cells) was found to express as a cleaved species which is secreted into the media. The material was purified by chelating purification and evaluated by the rosette/trimer HPLC-SEC assay using the Bio-Sil 250 SEC column with 2× PBS as mobile phase (FIG. 15). As this is a cleaved F containing a fusion peptide, it was expected that would be in the postfusion form and would form rosettes and migrate in the void volume similar to postfusion RSV F rosettes (FIG. 15A). If the cleaved F protein harboring a fusion peptide was folded in the prefusion form, one would expect the fusion peptide to be buried in the prefusion head region preventing rosette formation. Prefusion trimers should migrate in the included volume with a retention time similar to the RSV F postfusion trimer lacking the fusion peptide (FIG. 15B). RSV F HRA Disulfide T58C V164C was run on HPLC-SEC, and the majority of material migrated in the column void volume, indicating the material was aggregating or forming rosettes of postfusion F. A smaller portion of the protein migrated in the included volume with a retention time consistent with an F trimer, suggesting some material formed the desired stabilized prefusion F.

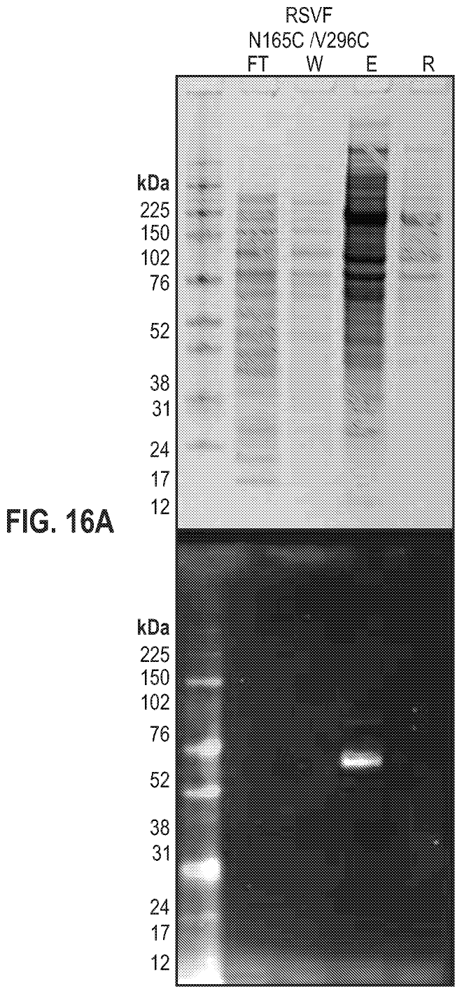

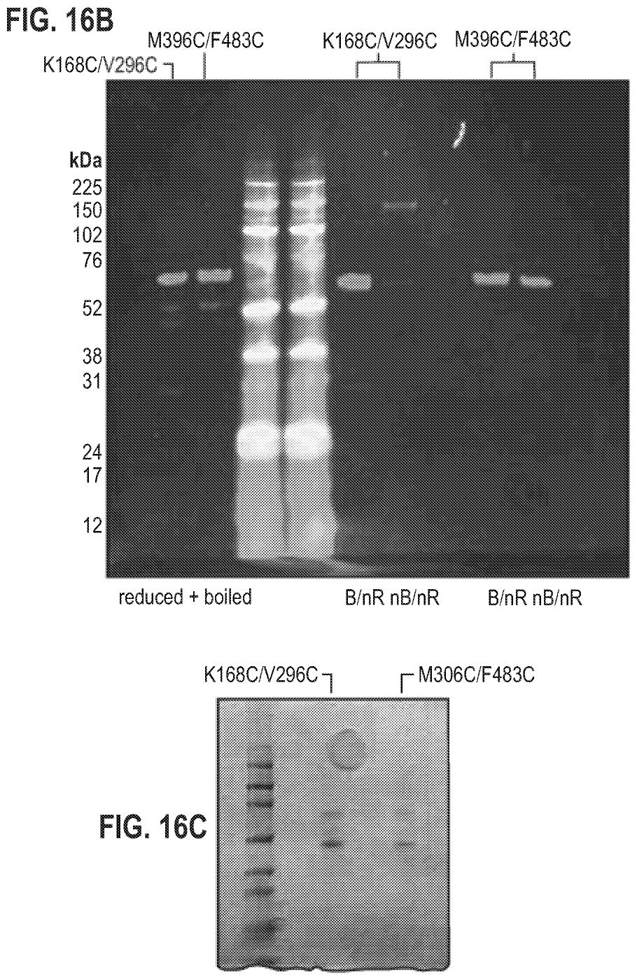

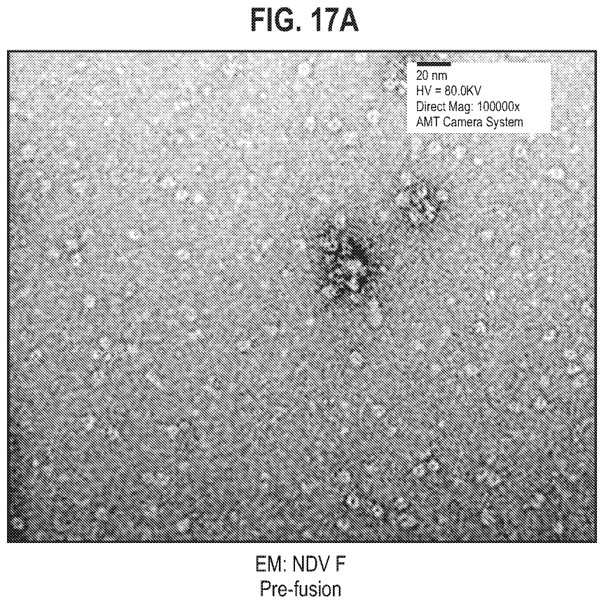

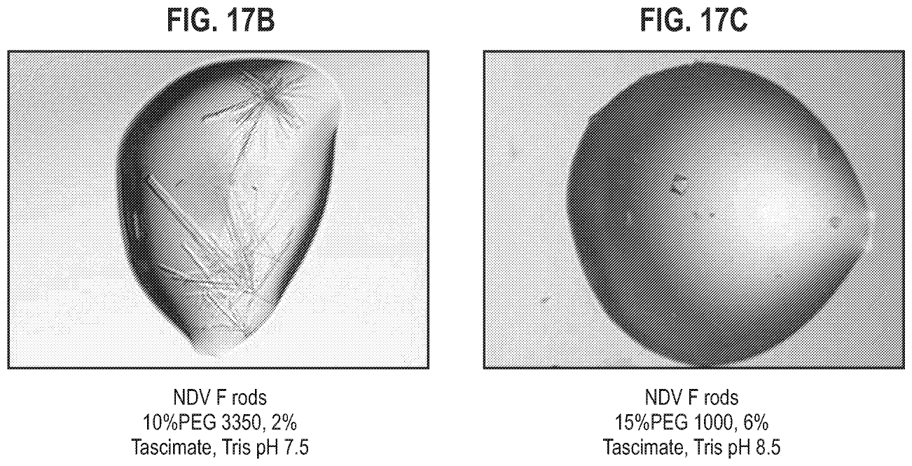

The disulfide constructs were subsequently cloned into baculovirus expression vectors and three constructs (HRA disulfides N165C/V296C and K168C/V296C and HRB M396C/F483C) were expressed. K168C/V296C and M396C/F483C, which were cleaved when expressed in mammalian cells, were secreted by insect cells predominantly as an uncleaved species (as shown by anti-HIS western blot). Both constructs migrated in the void fraction, which was inconsistent with previous observations that uncleaved species run as monomers. The proteins may have aggregated by virtue of incorrect disulfide formation. Gel shift analysis using anti-HIS western (FIG. 16) suggested that intra-chain disulfide bonds were not formed. The pure material taken from the void fraction of the SEC was analyzed with SDS-PAGE and coomassie staining, and each protein was found to be approximately 50% cleaved (FIG. 16). A third disulfide construct was expressed and N165C/V296C was secreted predominantly as the desired cleaved product as judged by western blot (FIG. 16A).

### 4. NDV F Prefusion Structure for Further Development of the RSV-NDV Chimera Subunit

A strategy for rescuing the RSV HRA prefusion epitopes was developed to generate an NDV F prefusion construct and mutate select residues of the HRA to those of RSV F. Initial attempts to replace the HRA of NDV with the HRA of RSV generated a construct which was not expressed/secreted from the cell. This indicated that the protein was misfolded. Further refinement of the residues of NDV F available for mutagenisis (i.e. those located on the protein surface) was required. A new construct, NDV F stabilized with a GCN trimerization domain (uncleaved) migrated as a trimer by SEC analysis. This was expected as this construct was shown to be a pre-fusion trimer by electron microscopy (Swanson et al, 2010).

5. EM Analysis of RSV F with HRB and Fusion Peptide Deletion Related to NDV Pre-Fusion Construct

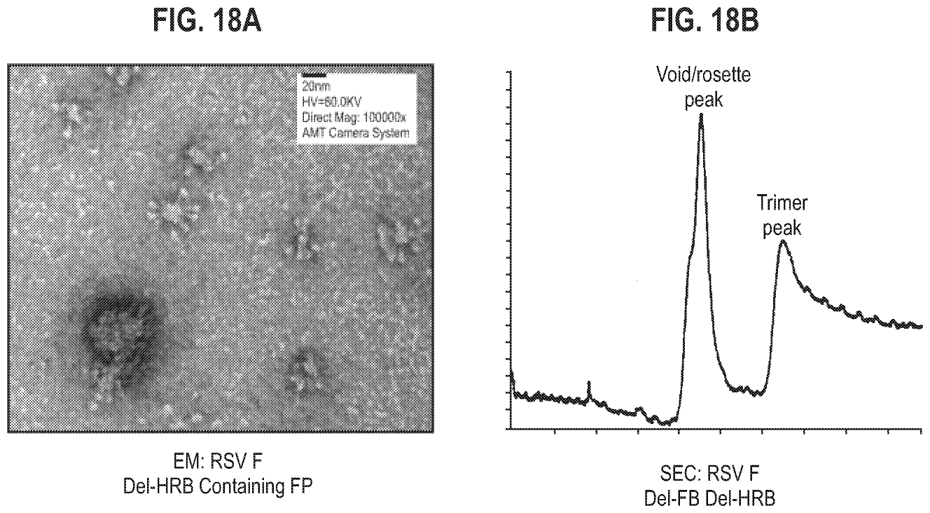

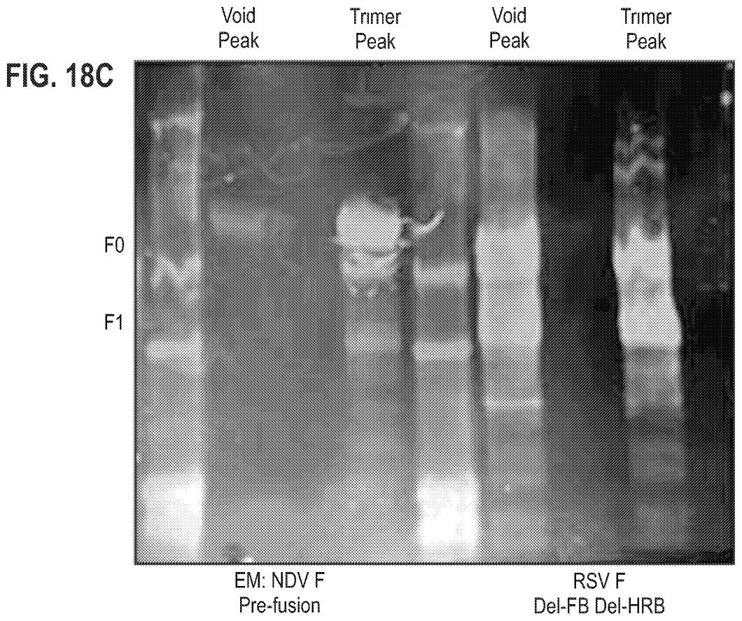

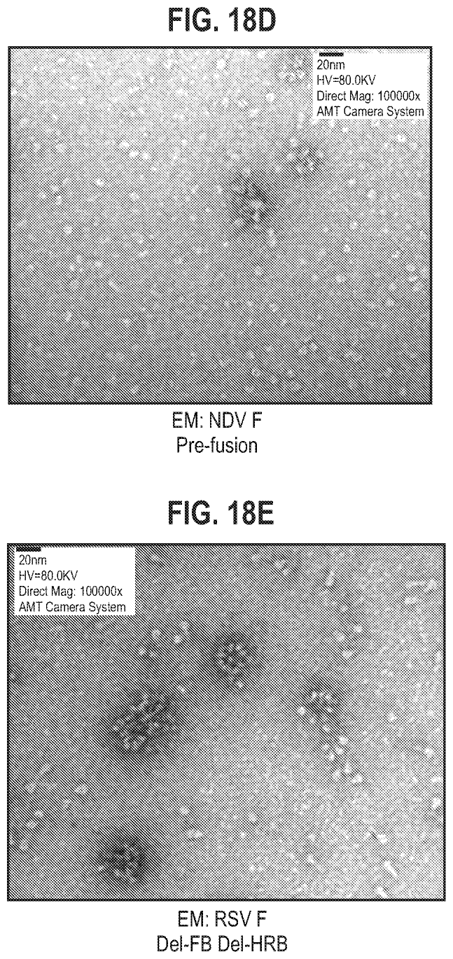

The HRB-deleted RSV F harboring the fusion-peptide was generated, and the protein was purified. The construct migrated in the void volume of the SEC column consistent with RSV F rosettes. The construct was evaluated by EM and shown to form rosettes similar to the postfusion RSV F (FIG. 18A). An RSV F protein with both the HRB and fusion peptide deleted (Del-HRB Del-FP) was generated, expressed and purified. RSV F with the HRB and fusion peptide deleted ran partially as a trimer on SEC (FIG. 18B). Gel analysis showed that RSV F del-HRB and Del-FP migrated both in the aggregation/rosette peak and trimer peak (FIG. 18C). For comparison, the uncleaved NDV pre-fusion construct had very little material in the void fraction (FIG. 18C). The RSV F Del-HRB Del-FP was collected from the trimer volume and evaluated by EM.

Electron micrographs of the NDV pre-fusion construct showed predominately the spherical heads expected for pre-fusion F (FIG. 18D). A portion of the material seemed to be associated in rosette-like aggregates, which should not be permitted as the construct is uncleaved and pre-fusion. This amount of association may be due to aggregation by the HIS-tag, and may explain why the RSV Del-HRB Del-FP contained some aggregate/rosette even when the fusion peptide was not present. EM analysis of RSV F Del-HRB Del-FP showed that the material was heterogeneous (FIG. 18E). RSV F Del-HRB Del-FP formed rosette-like structures similar to NDV F, as well as post-fusion like “crutches” and pre-fusion like “spheres”.

R057 deIHRB delFP trunc 6H
(fusion peptide deleted)
(SEQ ID NO: 10)
MELLILKANAITTILTAVTFCFASGQNITEEFYQS
TCSAVSKGYLSALRTGWYTSVITIELSNIKENKCN
GTDAKVKLIKQELDKYKNAVTELQLLMQSTPATNN
RARRELPRFMNYTLNNAKKTNVTLSKKRKRRSAIA
SGVAVSKVLHLEGEVNKIKSALLSTNKAVVSLSNG
VSVLTSKVLDLKNYIDKQLLPIVNKQSCSISNIET
VIEFQQKNNRLLEITREFSVNAGVTTPVSTYMLTN
SELLSLINDMPITNDQKKLMSNNVQIVRQQSYSIM
SIIKEEVLAYVVQLPLYGVIDTPCWKLHTSPLCTT
NTKEGSNICLTRTDRGWYCDNAGSVSFFPQAETCK
VQSNRVFCDTMNSLTLPSEVNLCNVDIFNPKYDCK
IMTSKTDVSSSVITSLGAIVSCYGKTKCTASNKNR
GIIKTFSNGCDYVSNKGVDTVSVGNTLYYVNKQEG
KSLYVKGEPIINFYDPLVFPSGGSAGSGHHHHHH**
R105 deIHRB delFP + linker trunk
6H (fusion peptide and linker deleted)
(SEQ ID NO: 38)
MELLILKANAITTILTAVTFCFASGQNITEEFYQS
TCSAVSKGYLSALRTGWYTSVITIELSNIKENKCN
GTDAKVKLIKQELDKYKNAVTELQLLMQSTPATNN
RARRELPRFMNYTLNNAKKTNVTLSKKRKRRSAIA
SGVAVSKVLHLEGEVNKIKSALLSTNKAVVSLSNG
VSVLTSKVLDLKNYIDKQLLPIVNKQSCSISNIET
VIEFQQKNNRLLEITREFSVNAGVTTPVSTYMLTN
SELLSLINDMPITNDQKKLMSNNVQIVRQQSYSIM
SIIKEEVLAYVVQLPLYGVIDTPCWKLHTSPLCTT
NTKEGSNICLTRTDRGWYCDNAGSVSFFPQAETCK
VQSNRVFCDTMNSLTLPSEVNLCNVDIFNPKYDCK
IMTSKTDVSSSVITSLGAIVSCYGKTKCTASNKNR
GIIKTFSNGCDYVSNKGVDTVSVGNTLYYVNKQEG
GGSAGSGHHHHHH**

### 6. Design of RSV F Non-Native HRB Constructs

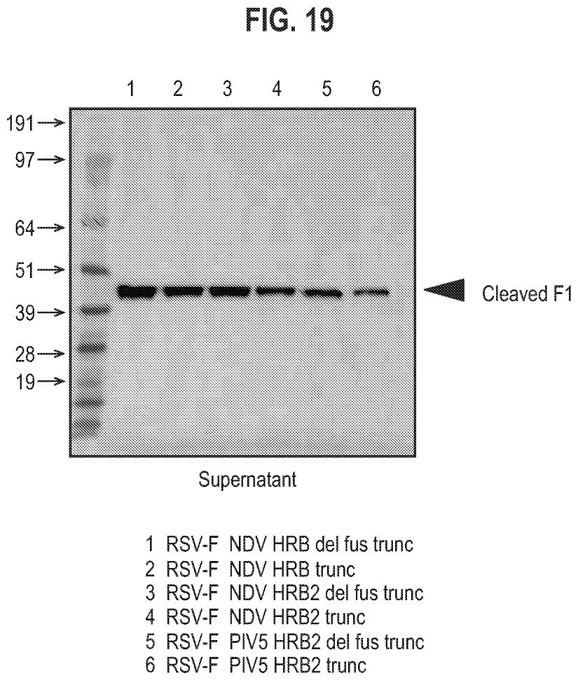

A stable RSV prefusion F was generated by replacing the HRB region with either the HRB region of NDV (with or without an additional glycine linker: HRB2) or PIV5. Mammalian expression of the non-native HRB constructs showed that each of the constructs were expressed and secreted well (FIG. 19). Additionally, the band observed migrated consistent with the cleaved F₁ species, suggesting that the proteins were properly processed. The constructs existed with or without fusion peptide (as indicated in FIG. 19).

### ADDITIONAL REFERENCES

The following references are hereby incorporated by reference for all that they teach.
1. Fields Virology. 4th edition, 2001.
2. Snell et al. (1997) Virus Genes 14:63-72.
3. Bembridge et al. (1999) J Virol 73: 10086-10094.
4. Li et al. (1998) J Exp Med 188:681-688
5. U.S. Pat. No. 6,060,308.
6. Yin et al. (2006) Nature 439:38-45.
7. Kim et al. (2007) J Med Virol 79: 820-828.
8. Yin et al. (2005) Proc Natl Acad Sci USA. 102(26):9288-93.
9. Chen et al. (2004) J Virol 78:4508-16.
10. Yang et at. (2002) J Virol 76:4634-42.
11. Harbury et al. (1993) Science 262:1401-1407.
12. Stevens et al. (2004) Science 303:1866-70.
13. Burkhard et al. (2001) Trends Cell Biol 11:82-88.
14. Section 5.5.2 of Proteins by Creighton (ISBN 0-7167-2317-4).
15. Yu (2002) Adv Drug Deliv Rev 54:1113-1129.
16. Muller et al. (2000) Methods Enzymol 328:261-282.
17. Beck & Brodsky (1998) J Struct Biol 122:17-29.
18. Lupas (1996) Trends Biochem Sci 21:375-382.
19. Adamson et al. (1993) Curr Opin Biotechnol 4:428-347.
20. Kammerer (1997) Matrix Biol 15:555-568.
21. Chao et al. (1998) J Chromatog B Biomed Sci Appl 715:307-329.
22. Arndt et al. (2002) Structure 10:1235-1248.
23. Liu & Lu (2002) J Biol Chem 277:48708-48713.
24. WO2006/011060.
25. Section 5.5.3 of Proteins by Creighton (ISBN 0-7167-2317-4).
26. Zhang & Chen (1999) J Biol Chem 274:22409-22413.
27. Slovic et al. (2003) Protein Sci 12:337-348
28. Gardner & Dutch (2007) J Virol 8 1:8303-14.
29. Gennaro (2000) Remington: The Science and Practice of Pharmacy. 20th edition, ISBN: 0683306472.
30. Nony et al. (2001) Vaccine 27:3645-51.
31. Greenbaum et al. (2004) Vaccine 22:2566-77.
32. Zurbriggen et al. (2003) Expert Rev Vaccines 2:295-304.
33. Piascik (2003) J Am Pharm Assoc (Wash DC). 43:728-30.
34. Mann et al. (2004) Vaccine 22:2425-9.
35. Halperin et al. (1979) Am J Public Health 69:1247-50.
36. Herbert et al. (1979) J Infect Dis 140:234-8.
37. Chen et al. (2003) Vaccine 21:2830-6.
38. U.S. Pat. No. 6,355,271.
39. WO00/23105.
40. U.S. Pat. No. 5,057,540.
41. WO96/33739.
42. EP-A-0109942.
43. WO96/11711.
44. WO00/07621.
45. Barr et al. (1998) Advanced Drug Delivery Reviews 32:247-271.
46. Sjolanderet et al. (1998) Advanced Drug Delivery Reviews 32:321-338.
47. Pizza et al. (2000) Int J Med Microbiol 290:455-461.
48. WO95/17211.
49. WO98/42375.
50. Singh et al (2001) J Cont Release 70:267-276.
51. WO99/27960.
52. U.S. Pat. No. 6,090,406.
53. U.S. Pat. No. 5,916,588.
54. EP-A-0626169.
55. WO99/52549.
56. WO01/21207.
57. WO01/21152.
58. Dyakonova et al. (2004) Int Immunopharmacol 4(13):1615-23.
59. FR-2859633.
60. Signorelli & Hadden (2003) Int Immunopharmacol 3(8):1177-86.
61. WO2004/064715.
62. De Libero et al, (2005) Nature Reviews Immunology 5:485-496
63. U.S. Pat. No. 5,936,076.
64. Old et al., J Clin Investig, 113:1631-1640
65. US2005/0192248
66. Yang et al. (2004) Angew Chem Int Ed 43:3818-3822
67. WO2005/102049.
68. Goffet et al (2004) Am Chem Soc 126:13602-13603
69. WO03/105769.
70. Cooper (1995) Pharm Biotechnol 6:559-80.
71. WO90/14837.
72. Podda & Del Giudice (2003) Expert Rev Vaccines 2:197-203.
73. Podda (2001) Vaccine 19: 2673-2680.
74. Vaccine Design: The Subunit and Adjuvant Approach (eds. Powell & Newman) Plenum Press 1995 (ISBN 0-306-44867-X).
75. Vaccine Adjuvants: Preparation Methods and Research Protocols (Volume 42 of Methods in Molecular Medicine series). ISBN: 1-59259-083-7. Ed. O'Hagan.
76. Allison & Byars (1992) Res Immunol 143:519-25.
77. Hariharan et al. (1995) Cancer Res 55:3486-9.
78. WO95/1 1700.
79. U.S. Pat. No. 6,080,725.
80. WO2005/0971 81.
81. Tassignon et al. (2005) J Immunol Meth 305:188-98.
82. Myers et al. (1990) pages 145-156 of Cellular and molecular aspects of endotoxin reactions.
83. Ulrich (2000) Chapter 16 (pages 273-282) of reference 75.
84. Johnson et al. (1999) J Med Chem 42:4640-9.
85. Baldrick et al. (2002) Regulatory Toxicol Pharmacol 35:398-413.
86. U.S. Pat. No. 4,680,338.
87. U.S. Pat. No. 4,988,815.
88. WO92/15582.
89. Stanley (2002) Clin Exp Dermatol 27:57 1-577.
90. Wu et al. (2004) Antiviral Res. 64(2):79-83.
91. Vasilakos et al. (2000) Cell Immunol. 204(1):64-74.
92. U.S. Pat. Nos. 4,689,338, 4,929,624, 5,238,944, 5,266,575, 5,268,376, 5,346,905, 5,352,784, 5,389,640, 5,395,937, 5,482,936, 5,494,916, 5,525,612, 6,083,505, 6,440,992, 6,627,640, 6,664,264, 6,664,265, 6,667,312, 6,677,347, 6,677,348, 6,677,349, 6,683,088, 6,703,402, 6,743,920, 6,800,624, 6,809,203, 6,888,000, and 6,924,293.
93. Jones (2003) Curr Opin Investig Drugs 4:214-218.
94. WO2004/060308.
95. WO2004/064759.
96. U.S. Pat. No. 6,924,271.
97. US2005/0070556.
98. U.S. Pat. No. 5,658,731.
99. U.S. Pat. No. 5,011,828.
100. WO2004/87 153.
101. U.S. Pat. No. 6,605,617.
102. WO02/18383.
103. WO2004/018455.
104. WO03/082272.
105. WO2006/002422.
106. Johnson et al. (1999) Bioorg Med Chem Lett 9:2273-2278.
107. Evans et al. (2003) Expert Rev Vaccines 2:219-229.
108. Andrianov et al. (1998) Biomaterials 19:109-115.
109. Payne et al. (1998) Adv Drug Delivery Review 31:185-196.
110. Thompson et al. (2003) Methods in Molecular Medicine 94:255-266.
111. Kandimalla et al. (2003) Nucleic Acids Research 31:2393-2400.
112. WO02/26757.
113. WO99/62923.
114. Krieg (2003) Nature Medicine 9:831-835.
115. McCluskie et al. (2002) FEMS Immunology and Medical Microbiology 32:179-185.
116. WO98/40100.
117. U.S. Pat. No. 6,207,646.
118. U.S. Pat. No. 6,239,116.
119. U.S. Pat. No. 6,429,199.
120. Kandimalla et al. (2003) Biochemical Society Transactions 31 (part 3): 654-658.
121. Blackwell et al. (2003) J Immunol 170:4061-4068.
122. Krieg (2002) Trends Immunol 23:64-65.
123. WO01/95935.
124. Kandimalla et al. (2003) BBRC 306:948-953.
125. Bhagat et al. (2003) BBRC 300:853-861.
126. WO03/035836.
127. WO01/22972.
128. Thompson et al. (2005) J Leukoc Biol 78: ‘The low-toxicity versions of LPS, MPL® adjuvant and RC529, are efficient adjuvants for CD4+ T cells’.
129. UK patent application GB-A-22202 11.
130. WO94/21292.
131. WO94/00153.
132. WO95/17210.
133. WO96/26741.
134. WO93/19780.
135. WO03/011223.
136. Meraldi et al. (2003) Vaccine 21:2485-249 1.
137. Pajak et al. (2003) Vaccine 21:836-842.
138. U.S. Pat. No. 6,586,409.
139. Wong et al. (2003) J Clin Pharmacol 43(7):735-42.
140. US2005/0215517.
141. McLellan J S, et al. (2010) Structural basis of respiratory syncytial virus neutralization by motavizumab. Nat Struct Mol Biol 17(2):248-250.
142. Ruiz-Arguello M B, et al. (2004) Thermostability of the human respiratory syncytial virus fusion protein before and after activation: implications for the membrane-fusion mechanism. J Gen Virol 85(Pt 12):3677-3687.
143. Calder L J, et al. (2000) Electron microscopy of the human respiratory syncytial virus fusion protein and complexes that it forms with monoclonal antibodies. Virology 271(1):122-131.
144. Connolly S A, Leser G P, Yin H S, Jardetzky T S, & Lamb R A (2006) Refolding of a paramyxovirus F protein from prefusion to postfusion conformations observed by liposome binding and electron microscopy, Proc Natl Acad Sci USA 103(47):17903-17908.
145. Yin H S, Wen X L, Paterson R G, Lamb R A, & Jardetzky T S (2006) Structure of the parainfluenza virus 5 F protein in its metastable, prefusion conformation, Nature 439(7072):38-44.
146. Swanson K, et al. (2010) Structure of the Newcastle disease virus F protein in the post-fusion conformation, Virology 402(2):372-379.
147. Yin H S, Wen X, Paterson R G, Lamb R A, & Jardetzky T S (2006) Structure of the parainfluenza virus 5 F protein in its metastable, prefusion conformation, Nature 439(7072):38-44.
148. McLellan J S, et al. (2010) Structure of a Major Antigenic Site on the Respiratory Syncytial Virus Fusion Glycoprotein in Complex with Neutralizing Antibody 101F. J Virol 84:12236-12244.
149. Arbiza J, et al. (1992) Characterization of two antigenic sites recognized by neutralizing monoclonal antibodies directed against the fusion glycoprotein of human respiratory syncytial virus. J Gen Virol 73:2225-2234.
150. Crowe J E, et al. (1998) Monoclonal antibody-resistant mutants selected with a respiratory syncytial virus-neutralizing human antibody fab fragment (Fab 19) define a unique epitope on the fusion (F) glycoprotein. Virology 252:373-375.
151. Lopez J A, Penas C, Garcia-Barreno B, Melero J A, & Portela A (1990) Location of a highly conserved neutralizing epitope in the F glycoprotein of human respiratory syncytial virus. J Virol 64:927-930.
152. Zhao X, Chen F P, & Sullender W M (2004) Respiratory syncytial virus escape mutant derived in vitro resists palivizumab prophylaxis in cotton rats. Virology 318:608-612.
153. Liu C, et al. (2007) Relationship between the loss of neutralizing antibody binding and fusion activity of the F protein of human respiratory syncytial virus. Virol J 4:71.
154. Lopez J A, et al. (1998) Antigenic structure of human respiratory syncytial virus fusion glycoprotein. J Virol 72:6922-6928.
155. Wu S J, et al. (2007) Characterization of the epitope for anti-human respiratory syncytial virus F protein monoclonal antibody 101F using synthetic peptides and genetic approaches. J Gen Virol 88:2719-2723.
156. Beeler J A & van Wyke Coelingh K (1989) Neutralization epitopes of the F glycoprotein of respiratory syncytial virus: effect of mutation upon fusion function. J Virol 63:2941-2950.

The entire teachings of all documents cited herein are hereby incorporated herein by reference.

(APPENDIX discloses SEQ ID NOS 29-32, 29-32 and 29-32, respectively, in order of appearance)

| HEADER | — | | | XX-XXX-9- xxxx | |
|---|---|---|---|---|---|
| COMPND | — | | | | |
| SSBOND | 1 | CYS G | 69 | CYS G | 212 |
| SSBOND | 2 | CYS G | 313 | CYS G | 343 |
| SSBOND | 3 | CYS G | 322 | CYS G | 333 |
| SSBOND | 4 | CYS G | 358 | CYS G | 367 |
| SSBOND | 5 | CYS G | 382 | CYS G | 393 |
| SSBOND | 6 | CYS G | 416 | CYS G | 422 |

| | | | | | | |
|---|---|---|---|---|---|---|
| LINKR | C1 | NAG J1535 | ND2 | ASN G | 70 | NAG-ASN |
| LINKR | C1 | NAG J1545 | ND2 | ASN G | 500 | NAG-ASN |
| LINKR | C1 | NAG J1525 | ND2 | ASN G | 27 | NAG-ASN |

| | | | | | |
|---|---|---|---|---|---|
| SSBOND | 7 | CYS H | 69 | CYS H | 212 |
| SSBOND | 8 | CYS H | 313 | CYS H | 343 |
| SSBOND | 9 | CYS H | 322 | CYS H | 333 |
| SSBOND | 10 | CYS H | 358 | CYS H | 367 |
| SSBOND | 11 | CYS H | 382 | CYS H | 393 |
| SSBOND | 12 | CYS H | 416 | CYS H | 422 |

| | | | | | | |
|---|---|---|---|---|---|---|
| LINKR | C1 | NAG K1535 | ND2 | ASN H | 70 | NAG-ASN |

| | | | | | | |
|---|---|---|---|---|---|---|
| LINKR | OD1 | ASN H | 500 | C7 | NAG K1545 | ASN-NAG |

| | | | | | | |
|---|---|---|---|---|---|---|
| LINKR | C1 | NAG K1545 | ND2 | ASN H | 500 | NAG-ASN |

| | | | | | | |
|---|---|---|---|---|---|---|
| LINKR | OD1 | ASN H | 27 | O7 | NAG K1525 | ASN-NAG1 |

| | | | | | | |
|---|---|---|---|---|---|---|
| LINKR | C1 | NAG K1525 | ND2 | ASN H | 27 | NAG-ASN |

| | | | | | |
|---|---|---|---|---|---|
| SSBOND | 13 | CYS I | 69 | CYS I | 212 |
| SSBOND | 14 | CYS I | 313 | CYS I | 343 |
| SSBOND | 15 | CYS I | 322 | CYS I | 333 |
| SSBOND | 16 | CYS I | 358 | CYS I | 367 |
| SSBOND | 17 | CYS I | 382 | CYS I | 393 |
| SSBOND | 18 | CYS I | 416 | CYS I | 422 |

| | | | | | | |
|---|---|---|---|---|---|---|
| LINKR | C1 | NAG L1535 | ND2 | ASN I | 70 | NAG-ASN |
| LINKR | C1 | NAG L1545 | ND2 | ASN I | 500 | NAG-ASN |
| LINKR | C1 | NAG L1525 | ND2 | ASN I | 27 | NAG-ASN |

| | | | | | |
|---|---|---|---|---|---|
| LINKR | GLN G | 98 | PHE G | 137 | gap |
| LINKR | ASN G | 325 | SER G | 330 | gap |
| LINKR | LYS G | 465 | GLU G | 472 | gap |

| | | | |
|---|---|---|---|
| LINKR | NAG J1535 | NAG J1536 | BETA1-4 |
| LINKR | NAG J1545 | NAG J1546 | BETA1-4 |
| LINKR | NAG J1525 | NAG J1526 | BETA1-4 |

| | | | | | |
|---|---|---|---|---|---|
| LINKR | PRO G | 304 | TYR G | 306 | gap |
| LINKR | THR G | 50 | ILE G | 309 | gap |
| LINKR | GLN H | 98 | PHE H | 137 | gap |
| LINKR | ASN H | 325 | SER H | 330 | gap |
| LINKR | LYS H | 465 | GLU H | 472 | gap |
| LINKR | PRO H | 304 | TYR H | 306 | gap |
| LINKR | THR H | 50 | ILE H | 309 | gap |
| LINKR | GLN I | 98 | PHE I | 137 | gap |
| LINKR | ASN I | 325 | SER I | 330 | gap |
| LINKR | LYS I | 465 | GLU I | 472 | gap |
| LINKR | PRO I | 304 | TYR I | 306 | gap |
| LINKR | THR I | 50 | ILE I | 309 | gap |

| | | | |
|---|---|---|---|
| LINKR | NAG K1535 | NAG K1536 | BETA1-4 |
| LINKR | NAG L1535 | NAG L1536 | BETA1-4 |
| LINKR | NAG K1545 | NAG K1546 | BETA1-4 |
| LINKR | NAG L1545 | NAG L1546 | BETA1-4 |
| LINKR | NAG K1525 | NAG K1526 | BETA1-4 |
| LINKR | NAG L1525 | NAG L1526 | BETA1-4 |

| | | | |
|---|---|---|---|
| MODRES | NAG J 1535 | NAG-b-D | RENAME |
| MODRES | NAG J 1536 | NAG-b-D | RENAME |
| MODRES | NAG J 1545 | NAG-b-D | RENAME |
| MODRES | NAG J 1546 | NAG-b-D | RENAME |
| MODRES | NAG J 1525 | NAG-b-D | RENAME |
| MODRES | NAG J 1526 | NAG-b-D | RENAME |
| MODRES | NAG K 1535 | NAG-b-D | RENAME |
| MODRES | NAG K 1536 | NAG-b-D | RENAME |
| MODRES | NAG K 1545 | NAG-b-D | RENAME |
| MODRES | NAG K 1546 | NAG-b-D | RENAME |
| MODRES | NAG K 1525 | NAG-b-D | RENAME |
| MODRES | NAG K 1526 | NAG-b-D | RENAME |
| MODRES | NAG L 1535 | NAG-b-D | RENAME |
| MODRES | NAG L 1536 | NAG-b-D | RENAME |
| MODRES | NAG L 1545 | NAG-b-D | RENAME |
| MODRES | NAG L 1546 | NAG-b-D | RENAME |
| MODRES | NAG L 1525 | NAG-b-D | RENAME |
| MODRES | NAG L 1526 | NAG-b-D | RENAME |

| | | | | | | | |
|---|---|---|---|---|---|---|---|
| CRYST1 | 160.206 | 259.864 | 154.012 | 90.00 | 90.00 | 90.00 | C 2 2 21 |

| | | | | |
|---|---|---|---|---|
| SCALE1 | 0.006242 | 0.000000 | 0.000000 | 0.00000 |
| SCALE2 | 0.000000 | 0.003848 | 0.000000 | 0.00000 |
| SCALE3 | 0.000000 | 0.000000 | 0.006493 | 0.00000 |

| | | | | | | | | | | | |
|---|---|---|---|---|---|---|---|---|---|---|---|
| ATOM | 1 | N | GLN G | 26 | 24.299 | 30.940 | −8.771 | 1.00 | 23.62 | | N |
| ATOM | 2 | CA | GLN G | 26 | 22.913 | 30.470 | −9.055 | 1.00 | 23.82 | | C |
| ATOM | 3 | CB | GLN G | 26 | 22.260 | 29.923 | −7.783 | 1.00 | 23.94 | | C |
| ATOM | 4 | CG | GLN G | 26 | 23.103 | 28.894 | −7.043 | 1.00 | 28.83 | | C |
| ATOM | 5 | CD | GLN G | 26 | 24.309 | 29.508 | −6.355 | 1.00 | 36.36 | | C |
| ATOM | 6 | OE1 | GLN G | 26 | 25.367 | 29.671 | −6.963 | 1.00 | 39.09 | | O |
| ATOM | 7 | NE2 | GLN G | 26 | 24.176 | 29.785 | −5.063 | 1.00 | 37.86 | | N |
| ATOM | 8 | C | GLN G | 26 | 22.064 | 31.593 | −9.645 | 1.00 | 23.09 | | C |
| ATOM | 9 | O | GLN G | 26 | 22.368 | 32.772 | −9.466 | 1.00 | 24.21 | | O |
| ATOM | 10 | N | ASN G | 27 | 21.079 | 31.214 | −10.452 | 1.00 | 20.98 | | N |
| ATOM | 11 | CA | ASN G | 27 | 20.182 | 32.172 | −11.089 | 1.00 | 19.70 | | C |
| ATOM | 12 | CB | ASN G | 27 | 20.879 | 32.850 | −12.269 | 1.00 | 20.41 | | C |
| ATOM | 13 | CG | ASN G | 27 | 19.973 | 33.815 | −13.014 | 1.00 | 23.56 | | C |
| ATOM | 14 | OD1 | ASN G | 27 | 19.456 | 34.775 | −12.440 | 1.00 | 24.42 | | O |
| ATOM | 15 | ND2 | ASN G | 27 | 19.784 | 33.561 | −14.310 | 1.00 | 27.70 | | N |
| ATOM | 16 | C | ASN G | 27 | 18.931 | 31.465 | −11.578 | 1.00 | 18.42 | | C |
| ATOM | 17 | O | ASN G | 27 | 18.705 | 31.356 | −12.783 | 1.00 | 18.47 | | O |
| ATOM | 18 | N | ILE G | 28 | 18.240 | 30.807 | −10.655 | 1.00 | 16.54 | | N |
| ATOM | 19 | CA | ILE G | 28 | 17.060 | 30.044 | −11.018 | 1.00 | 14.59 | | C |
| ATOM | 20 | CB | ILE G | 28 | 16.771 | 28.910 | −10.025 | 1.00 | 14.01 | | C |
| ATOM | 21 | CG1 | ILE G | 28 | 17.176 | 29.324 | −8.610 | 1.00 | 15.60 | | C |
| ATOM | 22 | CD1 | ILE G | 28 | 16.159 | 30.199 | −7.917 | 1.00 | 16.26 | | C |
| ATOM | 23 | CG2 | ILE G | 28 | 17.501 | 27.642 | −10.441 | 1.00 | 12.57 | | C |
| ATOM | 24 | C | ILE G | 28 | 15.843 | 30.942 | −11.157 | 1.00 | 13.86 | | C |
| ATOM | 25 | O | ILE G | 28 | 15.699 | 31.935 | −10.443 | 1.00 | 13.70 | | O |
| ATOM | 26 | N | THR G | 29 | 15.098 | 30.702 | −12.227 | 1.00 | 13.23 | | N |
| ATOM | 27 | CA | THR G | 29 | 13.968 | 31.532 | −12.592 | 1.00 | 12.65 | | C |
| ATOM | 28 | CB | THR G | 29 | 14.306 | 32.435 | −13.790 | 1.00 | 12.69 | | C |
| ATOM | 29 | OG1 | THR G | 29 | 15.728 | 32.530 | −13.936 | 1.00 | 12.62 | | O |
| ATOM | 30 | CG2 | THR G | 29 | 13.723 | 33.825 | −13.594 | 1.00 | 12.92 | | C |
| ATOM | 31 | C | THR G | 29 | 12.854 | 30.597 | −13.010 | 1.00 | 12.47 | | C |
| ATOM | 32 | O | THR G | 29 | 13.106 | 29.526 | −13.560 | 1.00 | 12.53 | | O |
| ATOM | 33 | N | GLU G | 30 | 11.622 | 31.050 | −12.847 | 1.00 | 11.98 | | N |
| ATOM | 34 | CA | GLU G | 30 | 10.500 | 30.392 | −13.480 | 1.00 | 11.68 | | C |
| ATOM | 35 | CB | GLU G | 30 | 9.587 | 29.781 | −12.416 | 1.00 | 11.98 | | C |
| ATOM | 36 | CG | GLU G | 30 | 8.848 | 28.524 | −12.856 | 1.00 | 13.38 | | C |
| ATOM | 37 | CD | GLU G | 30 | 8.295 | 27.732 | −11.682 | 1.00 | 16.03 | | C |
| ATOM | 38 | OE1 | GLU G | 30 | 7.552 | 28.313 | −10.863 | 1.00 | 16.65 | | O |
| ATOM | 39 | OE2 | GLU G | 30 | 8.577 | 26.519 | −11.596 | 1.00 | 17.06 | | O |
| ATOM | 40 | C | GLU G | 30 | 9.743 | 31.433 | −14.281 | 1.00 | 11.39 | | C |
| ATOM | 41 | O | GLU G | 30 | 9.521 | 32.547 | −13.806 | 1.00 | 11.50 | | O |
| ATOM | 42 | N | GLU G | 31 | 9.449 | 31.119 | −15.535 | 1.00 | 11.11 | | N |
| ATOM | 43 | CA | GLU G | 31 | 8.573 | 31.975 | −16.305 | 1.00 | 11.06 | | C |
| ATOM | 44 | CB | GLU G | 31 | 9.345 | 32.716 | −17.394 | 1.00 | 11.48 | | C |
| ATOM | 45 | CG | GLU G | 31 | 9.026 | 32.268 | −18.803 | 1.00 | 13.90 | | C |
| ATOM | 46 | CD | GLU G | 31 | 9.622 | 33.190 | −19.843 | 1.00 | 17.27 | | C |
| ATOM | 47 | OE1 | GLU G | 31 | 8.852 | 33.749 | −20.653 | 1.00 | 17.55 | | O |
| ATOM | 48 | OE2 | GLU G | 31 | 10.849 | 33.418 | −19.804 | 1.00 | 18.73 | | O |
| ATOM | 49 | C | GLU G | 31 | 7.370 | 31.240 | −16.871 | 1.00 | 10.47 | | C |
| ATOM | 50 | O | GLU G | 31 | 7.459 | 30.080 | −17.274 | 1.00 | 10.12 | | O |
| ATOM | 51 | N | PHE G | 32 | 6.220 | 31.890 | −16.753 | 1.00 | 10.15 | | N |
| ATOM | 52 | CA | PHE G | 32 | 4.932 | 31.235 | −16.887 | 1.00 | 9.62 | | C |
| ATOM | 53 | CB | PHE G | 32 | 3.967 | 31.777 | −15.832 | 1.00 | 9.42 | | C |
| ATOM | 54 | CG | PHE G | 32 | 2.524 | 31.512 | −16.141 | 1.00 | 9.27 | | C |
| ATOM | 55 | CD1 | PHE G | 32 | 2.021 | 30.222 | −16.085 | 1.00 | 9.37 | | C |
| ATOM | 56 | CE1 | PHE G | 32 | 0.701 | 29.960 | −16.407 | 1.00 | 9.52 | | C |
| ATOM | 57 | CZ | PHE G | 32 | −0.132 | 30.993 | −16.800 | 1.00 | 9.82 | | C |
| ATOM | 58 | CE2 | PHE G | 32 | 0.362 | 32.285 | −16.877 | 1.00 | 10.13 | | C |
| ATOM | 59 | CD2 | PHE G | 32 | 1.681 | 32.541 | −16.536 | 1.00 | 10.06 | | C |
| ATOM | 60 | C | PHE G | 32 | 4.386 | 31.524 | −18.276 | 1.00 | 9.64 | | C |
| ATOM | 61 | O | PHE G | 32 | 4.479 | 32.650 | −18.763 | 1.00 | 9.84 | | O |
| ATOM | 62 | N | TYR G | 33 | 3.846 | 30.501 | −18.926 | 1.00 | 9.59 | | N |
| ATOM | 63 | CA | TYR G | 33 | 3.342 | 30.662 | −20.281 | 1.00 | 9.67 | | C |
| ATOM | 64 | CB | TYR G | 33 | 3.951 | 29.617 | −21.212 | 1.00 | 9.52 | | C |
| ATOM | 65 | CG | TYR G | 33 | 5.422 | 29.834 | −21.467 | 1.00 | 10.94 | | C |
| ATOM | 66 | CD1 | TYR G | 33 | 6.379 | 29.220 | −20.670 | 1.00 | 12.23 | | C |
| ATOM | 67 | CE1 | TYR G | 33 | 7.726 | 29.448 | −20.870 | 1.00 | 12.93 | | C |
| ATOM | 68 | CZ | TYR G | 33 | 8.128 | 30.348 | −21.833 | 1.00 | 13.47 | | C |
| ATOM | 69 | OH | TYR G | 33 | 9.469 | 30.588 | −22.028 | 1.00 | 14.54 | | O |
| ATOM | 70 | CE2 | TYR G | 33 | 7.195 | 31.019 | −22.594 | 1.00 | 13.56 | | C |
| ATOM | 71 | CD2 | TYR G | 33 | 5.850 | 30.781 | −22.388 | 1.00 | 12.96 | | C |
| ATOM | 72 | C | TYR G | 33 | 1.824 | 30.604 | −20.327 | 1.00 | 9.94 | | C |
| ATOM | 73 | O | TYR G | 33 | 1.234 | 29.530 | −20.446 | 1.00 | 10.16 | | O |
| ATOM | 74 | N | GLN G | 34 | 1.199 | 31.769 | −20.211 | 1.00 | 10.06 | | N |
| ATOM | 75 | CA | GLN G | 34 | −0.225 | 31.846 | −19.927 | 1.00 | 10.01 | | C |
| ATOM | 76 | CB | GLN G | 34 | −0.649 | 33.302 | −19.751 | 1.00 | 10.22 | | C |
| ATOM | 77 | CG | GLN G | 34 | −2.138 | 33.486 | −19.551 | 1.00 | 11.12 | | C |
| ATOM | 78 | CD | GLN G | 34 | −2.485 | 34.867 | −19.039 | 1.00 | 13.13 | | C |
| ATOM | 79 | OE1 | GLN G | 34 | −2.668 | 35.804 | −19.819 | 1.00 | 14.24 | | O |
| ATOM | 80 | NE2 | GLN G | 34 | −2.553 | 35.007 | −17.719 | 1.00 | 13.79 | | N |
| ATOM | 81 | C | GLN G | 34 | −1.039 | 31.198 | −21.039 | 1.00 | 9.94 | | C |
| ATOM | 82 | O | GLN G | 34 | −2.165 | 30.752 | −20.819 | 1.00 | 10.08 | | O |
| ATOM | 83 | N | SER G | 35 | −0.468 | 31.167 | −22.238 | 1.00 | 9.87 | | N |
| ATOM | 84 | CA | SER G | 35 | −1.185 | 30.690 | −23.415 | 1.00 | 9.91 | | C |
| ATOM | 85 | CB | SER G | 35 | −0.619 | 31.337 | −24.684 | 1.00 | 10.03 | | C |
| ATOM | 86 | OG | SER G | 35 | 0.776 | 31.572 | −24.564 | 1.00 | 10.81 | | O |
| ATOM | 87 | C | SER G | 35 | −1.119 | 29.167 | −23.518 | 1.00 | 9.56 | | C |
| ATOM | 88 | O | SER G | 35 | −1.688 | 28.566 | −24.431 | 1.00 | 9.59 | | O |
| ATOM | 89 | N | THR G | 36 | −0.417 | 28.553 | −22.572 | 1.00 | 9.21 | | N |
| ATOM | 90 | CA | THR G | 36 | −0.209 | 27.113 | −22.573 | 1.00 | 9.06 | | C |
| ATOM | 91 | CB | THR G | 36 | 1.230 | 26.755 | −23.010 | 1.00 | 9.49 | | C |
| ATOM | 92 | OG1 | THR G | 36 | 1.493 | 27.310 | −24.305 | 1.00 | 10.82 | | O |
| ATOM | 93 | CG2 | THR G | 36 | 1.410 | 25.245 | −23.074 | 1.00 | 9.45 | | C |
| ATOM | 94 | C | THR G | 36 | −0.455 | 26.599 | −21.162 | 1.00 | 8.77 | | C |
| ATOM | 95 | O | THR G | 36 | −0.171 | 25.445 | −20.845 | 1.00 | 8.60 | | O |
| ATOM | 96 | N | CYS G | 37 | −0.982 | 27.478 | −20.315 | 1.00 | 8.69 | | N |
| ATOM | 97 | CA | CYS G | 37 | −1.097 | 27.201 | −18.890 | 1.00 | 8.69 | | C |
| ATOM | 98 | CB | CYS G | 37 | −2.468 | 26.605 | −18.571 | 1.00 | 8.81 | | C |
| ATOM | 99 | SG | CYS G | 37 | −3.003 | 26.848 | −16.864 | 1.00 | 10.80 | | S |
| ATOM | 100 | C | CYS G | 37 | 0.005 | 26.263 | −18.414 | 1.00 | 8.27 | | C |
| ATOM | 101 | O | CYS G | 37 | −0.268 | 25.166 | −17.926 | 1.00 | 8.27 | | O |
| ATOM | 102 | N | SER G | 38 | 1.251 | 26.687 | −18.586 | 1.00 | 7.92 | | N |
| ATOM | 103 | CA | SER G | 38 | 2.386 | 25.886 | −18.157 | 1.00 | 7.51 | | C |
| ATOM | 104 | CB | SER G | 38 | 2.817 | 24.924 | −19.264 | 1.00 | 7.63 | | C |
| ATOM | 105 | OG | SER G | 38 | 3.392 | 25.627 | −20.351 | 1.00 | 8.54 | | O |
| ATOM | 106 | C | SER G | 38 | 3.558 | 26.757 | −17.735 | 1.00 | 7.02 | | C |
| ATOM | 107 | O | SER G | 38 | 3.663 | 27.917 | −18.132 | 1.00 | 6.83 | | O |
| ATOM | 108 | N | ALA G | 39 | 4.407 | 26.198 | −16.882 | 1.00 | 6.64 | | N |
| ATOM | 109 | CA | ALA G | 39 | 5.466 | 26.961 | −16.245 | 1.00 | 6.58 | | C |
| ATOM | 110 | CB | ALA G | 39 | 5.136 | 27.195 | −14.782 | 1.00 | 6.67 | | C |
| ATOM | 111 | C | ALA G | 39 | 6.789 | 26.222 | −16.382 | 1.00 | 6.93 | | C |
| ATOM | 112 | O | ALA G | 39 | 6.842 | 24.998 | −16.261 | 1.00 | 7.38 | | O |
| ATOM | 113 | N | VAL G | 40 | 7.843 | 26.966 | −16.698 | 1.00 | 6.87 | | N |
| ATOM | 114 | CA | VAL G | 40 | 9.175 | 26.388 | −16.797 | 1.00 | 6.55 | | C |
| ATOM | 115 | CB | VAL G | 40 | 9.741 | 26.507 | −18.222 | 1.00 | 6.22 | | C |
| ATOM | 116 | CG1 | VAL G | 40 | 11.237 | 26.233 | −18.223 | 1.00 | 6.52 | | C |
| ATOM | 117 | CG2 | VAL G | 40 | 9.018 | 25.553 | −19.160 | 1.00 | 6.62 | | C |
| ATOM | 118 | C | VAL G | 40 | 10.140 | 27.033 | −15.813 | 1.00 | 6.73 | | C |
| ATOM | 119 | O | VAL G | 40 | 10.302 | 28.253 | −15.789 | 1.00 | 6.83 | | O |
| ATOM | 120 | N | SER G | 41 | 10.772 | 26.198 | −14.997 | 1.00 | 6.86 | | N |
| ATOM | 121 | CA | SER G | 41 | 11.897 | 26.625 | −14.181 | 1.00 | 6.75 | | C |
| ATOM | 122 | CB | SER G | 41 | 11.974 | 25.785 | −12.905 | 1.00 | 7.06 | | C |
| ATOM | 123 | OG | SER G | 41 | 10.751 | 25.110 | −12.666 | 1.00 | 7.50 | | O |
| ATOM | 124 | C | SER G | 41 | 13.206 | 26.525 | −14.958 | 1.00 | 6.56 | | C |
| ATOM | 125 | O | SER G | 41 | 13.526 | 25.482 | −15.529 | 1.00 | 6.08 | | O |
| ATOM | 126 | N | LYS G | 42 | 13.972 | 27.612 | −14.946 | 1.00 | 7.02 | | N |
| ATOM | 127 | CA | LYS G | 42 | 15.140 | 27.757 | −15.809 | 1.00 | 7.83 | | C |
| ATOM | 128 | CB | LYS G | 42 | 14.949 | 28.933 | −16.769 | 1.00 | 8.15 | | C |
| ATOM | 129 | CG | LYS G | 42 | 13.666 | 28.888 | −17.578 | 1.00 | 9.75 | | C |
| ATOM | 130 | CD | LYS G | 42 | 13.510 | 30.148 | −18.414 | 1.00 | 12.62 | | C |
| ATOM | 131 | CE | LYS G | 42 | 12.360 | 30.022 | −19.398 | 1.00 | 14.04 | | C |
| ATOM | 132 | NZ | LYS G | 42 | 12.643 | 30.734 | −20.675 | 1.00 | 14.96 | | N |
| ATOM | 133 | C | LYS G | 42 | 16.394 | 27.988 | −14.971 | 1.00 | 7.80 | | C |
| ATOM | 134 | O | LYS G | 42 | 16.323 | 28.557 | −13.882 | 1.00 | 8.15 | | O |
| ATOM | 135 | N | GLY G | 43 | 17.550 | 27.717 | −15.566 | 1.00 | 7.59 | | N |
| ATOM | 136 | CA | GLY G | 43 | 18.820 | 28.160 | −15.002 | 1.00 | 7.35 | | C |
| ATOM | 137 | C | GLY G | 43 | 19.529 | 27.058 | −14.240 | 1.00 | 7.10 | | C |
| ATOM | 138 | O | GLY G | 43 | 20.294 | 27.323 | −13.313 | 1.00 | 7.13 | | O |
| ATOM | 139 | N | TYR G | 44 | 19.309 | 25.819 | −14.668 | 1.00 | 7.01 | | N |
| ATOM | 140 | CA | TYR G | 44 | 19.922 | 24.656 | −14.033 | 1.00 | 6.89 | | C |
| ATOM | 141 | CB | TYR G | 44 | 18.878 | 23.561 | −13.815 | 1.00 | 6.88 | | C |
| ATOM | 142 | CG | TYR G | 44 | 17.809 | 23.928 | −12.816 | 1.00 | 6.41 | | C |
| ATOM | 143 | CD1 | TYR G | 44 | 16.553 | 24.343 | −13.236 | 1.00 | 6.62 | | C |
| ATOM | 144 | CE1 | TYR G | 44 | 15.580 | 24.703 | −12.325 | 1.00 | 6.48 | | C |
| ATOM | 145 | CZ | TYR G | 44 | 15.862 | 24.662 | −10.976 | 1.00 | 6.88 | | C |
| ATOM | 146 | OH | TYR G | 44 | 14.897 | 25.025 | −10.065 | 1.00 | 7.50 | | O |
| ATOM | 147 | CE2 | TYR G | 44 | 17.110 | 24.279 | −10.536 | 1.00 | 6.68 | | C |
| ATOM | 148 | CD2 | TYR G | 44 | 18.077 | 23.922 | −11.454 | 1.00 | 5.91 | | C |
| ATOM | 149 | C | TYR G | 44 | 21.060 | 24.106 | −14.886 | 1.00 | 6.86 | | C |
| ATOM | 150 | O | TYR G | 44 | 21.012 | 24.190 | −16.113 | 1.00 | 7.14 | | O |
| ATOM | 151 | N | LEU G | 45 | 21.979 | 23.385 | −14.249 | 1.00 | 6.44 | | N |
| ATOM | 152 | CA | LEU G | 45 | 23.146 | 22.842 | −14.946 | 1.00 | 6.12 | | C |
| ATOM | 153 | CB | LEU G | 45 | 24.425 | 23.552 | −14.482 | 1.00 | 6.31 | | C |
| ATOM | 154 | CG | LEU G | 45 | 24.534 | 25.052 | −14.775 | 1.00 | 6.38 | | C |
| ATOM | 155 | CD1 | LEU G | 45 | 25.624 | 25.690 | −13.928 | 1.00 | 7.05 | | C |
| ATOM | 156 | CD2 | LEU G | 45 | 24.787 | 25.299 | −16.255 | 1.00 | 7.05 | | C |
| ATOM | 157 | C | LEU G | 45 | 23.279 | 21.312 | −14.800 | 1.00 | 5.84 | | C |
| ATOM | 158 | O | LEU G | 45 | 23.333 | 20.797 | −13.683 | 1.00 | 5.94 | | O |
| ATOM | 159 | N | SER G | 46 | 23.470 | 20.620 | −15.929 | 1.00 | 5.43 | | N |
| ATOM | 160 | CA | SER G | 46 | 23.249 | 19.157 | −16.047 | 1.00 | 5.26 | | C |
| ATOM | 161 | CB | SER G | 46 | 22.913 | 18.795 | −17.498 | 1.00 | 5.21 | | C |
| ATOM | 162 | OG | SER G | 46 | 24.090 | 18.532 | −18.243 | 1.00 | 4.95 | | O |
| ATOM | 163 | C | SER G | 46 | 24.481 | 18.355 | −15.585 | 1.00 | 5.51 | | C |
| ATOM | 164 | O | SER G | 46 | 25.480 | 18.961 | −15.200 | 1.00 | 6.03 | | O |
| ATOM | 165 | N | ALA G | 47 | 24.453 | 17.015 | −15.656 | 1.00 | 5.24 | | N |
| ATOM | 166 | CA | ALA G | 47 | 25.584 | 16.224 | −15.104 | 1.00 | 4.83 | | C |
| ATOM | 167 | CB | ALA G | 47 | 25.690 | 16.412 | −13.599 | 1.00 | 5.01 | | C |
| ATOM | 168 | C | ALA G | 47 | 25.854 | 14.751 | −15.494 | 1.00 | 4.35 | | C |
| ATOM | 169 | O | ALA G | 47 | 24.926 | 13.972 | −15.719 | 1.00 | 4.45 | | O |
| ATOM | 170 | N | LEU G | 48 | 27.119 | 14.348 | −15.323 | 1.00 | 3.67 | | N |
| ATOM | 171 | CA | LEU G | 48 | 27.564 | 12.948 | −15.451 | 1.00 | 3.15 | | C |
| ATOM | 172 | CB | LEU G | 48 | 28.365 | 12.755 | −16.750 | 1.00 | 2.78 | | C |
| ATOM | 173 | CG | LEU G | 48 | 27.659 | 12.681 | −18.108 | 1.00 | 2.18 | | C |
| ATOM | 174 | CD1 | LEU G | 48 | 28.223 | 11.532 | −18.935 | 1.00 | 2.00 | | C |
| ATOM | 175 | CD2 | LEU G | 48 | 26.156 | 12.531 | −17.934 | 1.00 | 3.94 | | C |
| ATOM | 176 | C | LEU G | 48 | 28.443 | 12.517 | −14.259 | 1.00 | 3.06 | | C |
| ATOM | 177 | O | LEU G | 48 | 29.670 | 12.486 | −14.373 | 1.00 | 3.27 | | O |
| ATOM | 178 | N | ARG G | 49 | 27.810 | 12.061 | −13.177 | 1.00 | 2.91 | | N |
| ATOM | 179 | CA | ARG G | 49 | 28.509 | 11.788 | −11.915 | 1.00 | 2.88 | | C |
| ATOM | 180 | CB | ARG G | 49 | 27.538 | 11.277 | −10.847 | 1.00 | 2.69 | | C |
| ATOM | 181 | CG | ARG G | 49 | 28.117 | 11.273 | −9.440 | 1.00 | 2.15 | | C |
| ATOM | 182 | CD | ARG G | 49 | 28.015 | 9.897 | −8.806 | 1.00 | 2.00 | | C |
| ATOM | 183 | NE | ARG G | 49 | 29.322 | 9.271 | −8.623 | 1.00 | 5.18 | | N |
| ATOM | 184 | CZ | ARG G | 49 | 29.526 | 7.956 | −8.629 | 1.00 | 7.68 | | C |
| ATOM | 185 | NH1 | ARG G | 49 | 28.518 | 7.129 | −8.866 | 1.00 | 8.32 | | N |
| ATOM | 186 | NH2 | ARG G | 49 | 30.748 | 7.469 | −8.455 | 1.00 | 8.12 | | N |
| ATOM | 187 | C | ARG G | 49 | 29.678 | 10.816 | −12.065 | 1.00 | 3.09 | | C |
| ATOM | 188 | O | ARG G | 49 | 29.570 | 9.796 | −12.747 | 1.00 | 3.08 | | O |
| ATOM | 189 | N | THR G | 50 | 30.820 | 11.189 | −11.489 | 1.00 | 3.37 | | N |
| ATOM | 190 | CA | THR G | 50 | 32.099 | 10.564 | −11.823 | 1.00 | 3.55 | | C |
| ATOM | 191 | CB | THR G | 50 | 32.487 | 10.773 | −13.299 | 1.00 | 3.25 | | C |
| ATOM | 192 | OG1 | THR G | 50 | 32.842 | 12.144 | −13.519 | 1.00 | 2.66 | | O |
| ATOM | 193 | CG2 | THR G | 50 | 31.328 | 10.401 | −14.212 | 1.00 | 3.46 | | C |
| ATOM | 194 | C | THR G | 50 | 33.259 | 10.968 | −10.908 | 1.00 | 4.07 | | C |
| ATOM | 195 | O | THR G | 50 | 33.768 | 10.130 | −10.165 | 1.00 | 4.35 | | O |
| ATOM | 196 | N | GLY G | 51 | 33.818 | 12.158 | −11.108 | 1.00 | 4.24 | | N |
| ATOM | 197 | CA | GLY G | 51 | 34.942 | 12.544 | −10.272 | 1.00 | 4.78 | | C |
| ATOM | 198 | C | GLY G | 51 | 34.692 | 11.849 | −8.951 | 1.00 | 4.98 | | C |
| ATOM | 199 | O | GLY G | 51 | 33.618 | 12.018 | −8.375 | 1.00 | 5.00 | | O |
| ATOM | 200 | N | TRP G | 52 | 35.476 | 10.816 | −8.667 | 1.00 | 5.34 | | N |
| ATOM | 201 | CA | TRP G | 52 | 35.123 | 9.926 | −7.569 | 1.00 | 5.42 | | C |
| ATOM | 202 | CB | TRP G | 52 | 34.864 | 8.497 | −8.049 | 1.00 | 5.75 | | C |
| ATOM | 203 | CG | TRP G | 52 | 33.714 | 8.373 | −9.001 | 1.00 | 6.38 | | C |
| ATOM | 204 | CD1 | TRP G | 52 | 33.776 | 7.964 | −10.300 | 1.00 | 7.27 | | C |
| ATOM | 205 | NE1 | TRP G | 52 | 32.533 | 8.031 | −10.878 | 1.00 | 7.46 | | N |
| ATOM | 206 | CE2 | TRP G | 52 | 31.627 | 8.449 | −9.938 | 1.00 | 7.15 | | C |
| ATOM | 207 | CD2 | TRP G | 52 | 32.331 | 8.658 | −8.735 | 1.00 | 7.02 | | C |
| ATOM | 208 | CE3 | TRP G | 52 | 31.627 | 9.102 | −7.608 | 1.00 | 6.83 | | C |
| ATOM | 209 | CZ3 | TRP G | 52 | 30.261 | 9.289 | −7.711 | 1.00 | 6.73 | | C |
| ATOM | 210 | CH2 | TRP G | 52 | 29.589 | 9.062 | −8.922 | 1.00 | 7.13 | | C |
| ATOM | 211 | CZ2 | TRP G | 52 | 30.252 | 8.637 | −10.041 | 1.00 | 6.82 | | C |
| ATOM | 212 | C | TRP G | 52 | 36.148 | 9.926 | −6.456 | 1.00 | 5.34 | | C |
| ATOM | 213 | O | TRP G | 52 | 37.349 | 10.027 | −6.698 | 1.00 | 5.47 | | O |
| ATOM | 214 | N | TYR G | 53 | 35.670 | 9.629 | −5.256 | 1.00 | 5.29 | | N |
| ATOM | 215 | CA | TYR G | 53 | 36.528 | 9.560 | −4.093 | 1.00 | 5.58 | | C |
| ATOM | 216 | CB | TYR G | 53 | 36.260 | 10.754 | −3.176 | 1.00 | 5.51 | | C |
| ATOM | 217 | CG | TYR G | 53 | 37.079 | 10.756 | −1.910 | 1.00 | 6.59 | | C |
| ATOM | 218 | CD1 | TYR G | 53 | 38.312 | 11.391 | −1.858 | 1.00 | 8.25 | | C |
| ATOM | 219 | CE1 | TYR G | 53 | 39.043 | 11.436 | −0.685 | 1.00 | 9.40 | | C |
| ATOM | 220 | CZ | TYR G | 53 | 38.524 | 10.875 | 0.462 | 1.00 | 9.24 | | C |
| ATOM | 221 | OH | TYR G | 53 | 39.246 | 10.915 | 1.633 | 1.00 | 9.61 | | O |
| ATOM | 222 | CE2 | TYR G | 53 | 37.287 | 10.272 | 0.443 | 1.00 | 8.78 | | C |
| ATOM | 223 | CD2 | TYR G | 53 | 36.559 | 10.247 | −0.730 | 1.00 | 7.93 | | C |
| ATOM | 224 | C | TYR G | 53 | 36.286 | 8.245 | −3.364 | 1.00 | 5.74 | | C |
| ATOM | 225 | O | TYR G | 53 | 35.186 | 7.996 | −2.870 | 1.00 | 6.19 | | O |
| ATOM | 226 | N | THR G | 54 | 37.221 | 7.317 | −3.532 | 1.00 | 6.04 | | N |
| ATOM | 227 | CA | THR G | 54 | 37.132 | 6.013 | −2.888 | 1.00 | 6.67 | | C |
| ATOM | 228 | CB | THR G | 54 | 38.232 | 5.060 | −3.396 | 1.00 | 6.97 | | C |
| ATOM | 229 | OG1 | THR G | 54 | 37.876 | 4.562 | −4.691 | 1.00 | 7.51 | | O |
| ATOM | 230 | CG2 | THR G | 54 | 38.409 | 3.889 | −2.442 | 1.00 | 7.98 | | C |
| ATOM | 231 | C | THR G | 54 | 37.254 | 6.158 | −1.376 | 1.00 | 6.97 | | C |
| ATOM | 232 | O | THR G | 54 | 37.900 | 7.083 | −0.884 | 1.00 | 7.32 | | O |
| ATOM | 233 | N | SER G | 55 | 36.616 | 5.254 | −0.639 | 1.00 | 7.24 | | N |
| ATOM | 234 | CA | SER G | 55 | 37.025 | 4.966 | 0.732 | 1.00 | 7.65 | | C |
| ATOM | 235 | CB | SER G | 55 | 36.459 | 6.009 | 1.705 | 1.00 | 7.69 | | C |
| ATOM | 236 | OG | SER G | 55 | 36.690 | 5.632 | 3.053 | 1.00 | 7.51 | | O |
| ATOM | 237 | C | SER G | 55 | 36.629 | 3.561 | 1.171 | 1.00 | 8.03 | | C |
| ATOM | 238 | O | SER G | 55 | 35.510 | 3.109 | 0.924 | 1.00 | 8.43 | | O |
| ATOM | 239 | N | VAL G | 56 | 37.522 | 2.919 | 1.915 | 1.00 | 8.14 | | N |
| ATOM | 240 | CA | VAL G | 56 | 37.401 | 1.500 | 2.215 | 1.00 | 8.41 | | C |
| ATOM | 241 | CB | VAL G | 56 | 38.732 | 0.763 | 1.967 | 1.00 | 8.72 | | C |
| ATOM | 242 | CG1 | VAL G | 56 | 38.479 | −0.703 | 1.654 | 1.00 | 9.29 | | C |
| ATOM | 243 | CG2 | VAL G | 56 | 39.509 | 1.433 | 0.843 | 1.00 | 9.51 | | C |
| ATOM | 244 | C | VAL G | 56 | 37.005 | 1.312 | 3.673 | 1.00 | 8.19 | | C |
| ATOM | 245 | O | VAL G | 56 | 37.760 | 1.658 | 4.578 | 1.00 | 8.54 | | O |
| ATOM | 246 | N | ILE G | 57 | 35.784 | 0.845 | 3.898 | 1.00 | 7.91 | | N |
| ATOM | 247 | CA | ILE G | 57 | 35.255 | 0.779 | 5.249 | 1.00 | 7.64 | | C |
| ATOM | 248 | CB | ILE G | 57 | 33.912 | 1.510 | 5.372 | 1.00 | 7.35 | | C |
| ATOM | 249 | CG1 | ILE G | 57 | 34.063 | 2.964 | 4.918 | 1.00 | 7.25 | | C |
| ATOM | 250 | CD1 | ILE G | 57 | 32.922 | 3.860 | 5.343 | 1.00 | 8.42 | | C |
| ATOM | 251 | CG2 | ILE G | 57 | 33.404 | 1.449 | 6.804 | 1.00 | 7.52 | | C |
| ATOM | 252 | C | ILE G | 57 | 35.125 | −0.655 | 5.739 | 1.00 | 7.69 | | C |
| ATOM | 253 | O | ILE G | 57 | 34.629 | −1.527 | 5.025 | 1.00 | 7.79 | | O |
| ATOM | 254 | N | THR G | 58 | 35.726 | −0.917 | 6.894 | 1.00 | 7.85 | | N |
| ATOM | 255 | CA | THR G | 58 | 35.912 | −2.275 | 7.379 | 1.00 | 8.15 | | C |
| ATOM | 256 | CB | THR G | 58 | 37.405 | −2.631 | 7.469 | 1.00 | 8.14 | | C |
| ATOM | 257 | OG1 | THR G | 58 | 38.159 | −1.761 | 6.615 | 1.00 | 8.62 | | O |
| ATOM | 258 | CG2 | THR G | 58 | 37.634 | −4.075 | 7.053 | 1.00 | 7.94 | | C |
| ATOM | 259 | C | THR G | 58 | 35.291 | −2.414 | 8.763 | 1.00 | 8.20 | | C |
| ATOM | 260 | O | THR G | 58 | 35.589 | −1.633 | 9.664 | 1.00 | 8.18 | | O |
| ATOM | 261 | N | ILE G | 59 | 34.396 | −3.384 | 8.915 | 1.00 | 8.54 | | N |
| ATOM | 262 | CA | ILE G | 59 | 33.617 | −3.525 | 10.143 | 1.00 | 9.23 | | C |
| ATOM | 263 | CB | ILE G | 59 | 32.144 | −3.139 | 9.912 | 1.00 | 8.92 | | C |
| ATOM | 264 | CG1 | ILE G | 59 | 32.031 | −1.688 | 9.443 | 1.00 | 9.27 | | C |
| ATOM | 265 | CD1 | ILE G | 59 | 30.626 | −1.285 | 9.045 | 1.00 | 10.89 | | C |
| ATOM | 266 | CG2 | ILE G | 59 | 31.326 | −3.377 | 11.170 | 1.00 | 8.74 | | C |
| ATOM | 267 | C | ILE G | 59 | 33.648 | −4.973 | 10.621 | 1.00 | 10.02 | | C |
| ATOM | 268 | O | ILE G | 59 | 33.170 | −5.865 | 9.921 | 1.00 | 10.20 | | O |
| ATOM | 269 | N | GLU G | 60 | 34.251 | −5.221 | 11.779 | 1.00 | 10.95 | | N |
| ATOM | 270 | CA | GLU G | 60 | 34.630 | −6.586 | 12.133 | 1.00 | 12.13 | | C |
| ATOM | 271 | CB | GLU G | 60 | 35.311 | −6.605 | 13.510 | 1.00 | 12.44 | | C |
| ATOM | 272 | CG | GLU G | 60 | 36.583 | −5.750 | 13.558 | 1.00 | 15.04 | | C |
| ATOM | 273 | CD | GLU G | 60 | 37.500 | −6.089 | 14.724 | 1.00 | 19.34 | | C |
| ATOM | 274 | OE1 | GLU G | 60 | 37.163 | −5.716 | 15.869 | 1.00 | 20.63 | | O |
| ATOM | 275 | OE2 | GLU G | 60 | 38.625 | −6.578 | 14.476 | 1.00 | 21.19 | | O |
| ATOM | 276 | C | GLU G | 60 | 33.397 | −7.503 | 12.086 | 1.00 | 12.46 | | C |
| ATOM | 277 | O | GLU G | 60 | 32.369 | −7.164 | 12.677 | 1.00 | 12.68 | | O |
| ATOM | 278 | N | LEU G | 61 | 33.448 | −8.627 | 11.361 | 1.00 | 12.80 | | N |
| ATOM | 279 | CA | LEU G | 61 | 32.635 | −9.731 | 11.843 | 1.00 | 12.71 | | C |
| ATOM | 280 | CB | LEU G | 61 | 32.816 | −11.079 | 11.148 | 1.00 | 12.55 | | C |
| ATOM | 281 | CG | LEU G | 61 | 31.902 | −12.059 | 11.912 | 1.00 | 12.39 | | C |
| ATOM | 282 | CD1 | LEU G | 61 | 30.438 | −11.897 | 11.514 | 1.00 | 13.14 | | C |
| ATOM | 283 | CD2 | LEU G | 61 | 32.335 | −13.513 | 11.814 | 1.00 | 12.50 | | C |
| ATOM | 284 | C | LEU G | 61 | 33.223 | −9.800 | 13.207 | 1.00 | 12.94 | | C |
| ATOM | 285 | O | LEU G | 61 | 34.388 | −10.187 | 13.351 | 1.00 | 12.78 | | O |
| ATOM | 286 | N | SER G | 62 | 32.676 | −8.895 | 13.997 | 1.00 | 13.61 | | N |
| ATOM | 287 | CA | SER G | 62 | 32.941 | −8.871 | 15.409 | 1.00 | 14.70 | | C |
| ATOM | 288 | CB | SER G | 62 | 32.339 | −7.611 | 16.023 | 1.00 | 14.52 | | C |
| ATOM | 289 | OG | SER G | 62 | 32.798 | −6.455 | 15.345 | 1.00 | 14.80 | | O |
| ATOM | 290 | C | SER G | 62 | 32.342 | −10.111 | 16.051 | 1.00 | 15.48 | | C |
| ATOM | 291 | O | SER G | 62 | 31.132 | −10.187 | 16.263 | 1.00 | 15.50 | | O |
| ATOM | 292 | N | ASN G | 63 | 33.164 | −11.141 | 16.199 | 1.00 | 16.61 | | N |
| ATOM | 293 | CA | ASN G | 63 | 32.650 | −12.422 | 16.635 | 1.00 | 17.77 | | C |
| ATOM | 294 | CB | ASN G | 63 | 33.597 | −13.559 | 16.266 | 1.00 | 17.73 | | C |
| ATOM | 295 | CG | ASN G | 63 | 32.857 | −14.834 | 15.934 | 1.00 | 18.52 | | C |
| ATOM | 296 | OD1 | ASN G | 63 | 31.848 | −15.156 | 16.561 | 1.00 | 18.29 | | O |
| ATOM | 297 | ND2 | ASN G | 63 | 33.295 | −15.516 | 14.883 | 1.00 | 19.40 | | N |
| ATOM | 298 | C | ASN G | 63 | 32.325 | −12.448 | 18.119 | 1.00 | 18.53 | | C |
| ATOM | 299 | O | ASN G | 63 | 32.967 | −11.768 | 18.919 | 1.00 | 18.82 | | O |
| ATOM | 300 | N | ILE G | 64 | 31.240 | −13.135 | 18.457 | 1.00 | 19.60 | | N |
| ATOM | 301 | CA | ILE G | 64 | 30.912 | −13.413 | 19.848 | 1.00 | 21.04 | | C |
| ATOM | 302 | CB | ILE G | 64 | 29.403 | −13.648 | 20.038 | 1.00 | 20.60 | | C |
| ATOM | 303 | CG1 | ILE G | 64 | 28.593 | −12.612 | 19.259 | 1.00 | 20.13 | | C |
| ATOM | 304 | CD1 | ILE G | 64 | 27.150 | −12.497 | 19.722 | 1.00 | 20.19 | | C |
| ATOM | 305 | CG2 | ILE G | 64 | 29.040 | −13.615 | 21.514 | 1.00 | 20.74 | | C |
| ATOM | 306 | C | ILE G | 64 | 31.660 | −14.643 | 20.347 | 1.00 | 22.58 | | C |
| ATOM | 307 | O | ILE G | 64 | 31.672 | −15.682 | 19.687 | 1.00 | 23.07 | | O |
| ATOM | 308 | N | LYS G | 65 | 32.173 | −14.559 | 21.570 | 1.00 | 24.21 | | N |
| ATOM | 309 | CA | LYS G | 65 | 32.425 | −15.751 | 22.374 | 1.00 | 25.86 | | C |
| ATOM | 310 | CB | LYS G | 65 | 33.801 | −15.682 | 23.043 | 1.00 | 25.90 | | C |
| ATOM | 311 | CG | LYS G | 65 | 34.437 | −14.301 | 23.031 | 1.00 | 26.69 | | C |
| ATOM | 312 | CD | LYS G | 65 | 33.621 | −13.312 | 23.847 | 1.00 | 28.26 | | C |
| ATOM | 313 | CE | LYS G | 65 | 34.477 | −12.614 | 24.890 | 1.00 | 28.67 | | C |
| ATOM | 314 | NZ | LYS G | 65 | 33.661 | −12.120 | 26.034 | 1.00 | 28.95 | | N |
| ATOM | 315 | C | LYS G | 65 | 31.329 | −15.991 | 23.413 | 1.00 | 26.81 | | C |
| ATOM | 316 | O | LYS G | 65 | 31.299 | −15.345 | 24.461 | 1.00 | 26.72 | | O |
| ATOM | 317 | N | GLU G | 66 | 30.491 | −16.990 | 23.149 | 1.00 | 27.90 | | N |
| ATOM | 318 | CA | GLU G | 66 | 29.237 | −17.188 | 23.879 | 1.00 | 28.82 | | C |
| ATOM | 319 | CB | GLU G | 66 | 28.305 | −18.104 | 23.076 | 1.00 | 28.88 | | C |
| ATOM | 320 | CG | GLU G | 66 | 27.621 | −19.192 | 23.895 | 1.00 | 30.33 | | C |
| ATOM | 321 | CD | GLU G | 66 | 27.188 | −20.381 | 23.052 | 1.00 | 33.17 | | C |
| ATOM | 322 | OE1 | GLU G | 66 | 26.253 | −21.098 | 23.468 | 1.00 | 35.05 | | O |
| ATOM | 323 | OE2 | GLU G | 66 | 27.824 | −20.634 | 22.007 | 1.00 | 33.14 | | O |
| ATOM | 324 | C | GLU G | 66 | 29.494 | −17.783 | 25.263 | 1.00 | 29.12 | | C |
| ATOM | 325 | O | GLU G | 66 | 30.348 | −18.655 | 25.418 | 1.00 | 29.28 | | O |
| ATOM | 326 | N | ASN G | 67 | 28.773 | −17.301 | 26.272 | 1.00 | 29.32 | | N |
| ATOM | 327 | CA | ASN G | 67 | 29.094 | −17.661 | 27.650 | 1.00 | 29.39 | | C |
| ATOM | 328 | CB | ASN G | 67 | 29.462 | −16.429 | 28.477 | 1.00 | 29.62 | | C |
| ATOM | 329 | CG | ASN G | 67 | 30.963 | −16.231 | 28.580 | 1.00 | 29.84 | | C |
| ATOM | 330 | OD1 | ASN G | 67 | 31.651 | −17.000 | 29.252 | 1.00 | 29.93 | | O |
| ATOM | 331 | ND2 | ASN G | 67 | 31.494 | −15.324 | 27.768 | 1.00 | 29.22 | | N |
| ATOM | 332 | C | ASN G | 67 | 28.069 | −18.540 | 28.367 | 1.00 | 29.15 | | C |
| ATOM | 333 | O | ASN G | 67 | 27.324 | −18.070 | 29.227 | 1.00 | 29.06 | | O |
| ATOM | 334 | N | LYS G | 68 | 27.964 | −19.788 | 27.921 | 1.00 | 28.87 | | N |
| ATOM | 335 | CA | LYS G | 68 | 27.848 | −20.936 | 28.819 | 1.00 | 28.78 | | C |
| ATOM | 336 | CB | LYS G | 68 | 29.127 | −21.783 | 28.771 | 1.00 | 28.89 | | C |
| ATOM | 337 | CG | LYS G | 68 | 29.681 | −22.003 | 27.364 | 1.00 | 29.26 | | C |
| ATOM | 338 | CD | LYS G | 68 | 31.070 | −22.631 | 27.392 | 1.00 | 29.68 | | C |
| ATOM | 339 | CE | LYS G | 68 | 31.200 | −23.648 | 28.514 | 1.00 | 29.46 | | C |
| ATOM | 340 | NZ | LYS G | 68 | 32.473 | −24.417 | 28.425 | 1.00 | 29.58 | | N |
| ATOM | 341 | C | LYS G | 68 | 27.492 | −20.555 | 30.268 | 1.00 | 28.60 | | C |
| ATOM | 342 | O | LYS G | 68 | 28.346 | −20.603 | 31.154 | 1.00 | 28.64 | | O |
| ATOM | 343 | N | CYS G | 69 | 26.261 | −20.069 | 30.468 | 1.00 | 28.09 | | N |
| ATOM | 344 | CA | CYS G | 69 | 25.535 | −20.153 | 31.759 | 1.00 | 27.63 | | C |
| ATOM | 345 | CB | CYS G | 69 | 25.056 | −18.753 | 32.214 | 1.00 | 27.07 | | C |
| ATOM | 346 | SG | CYS G | 69 | 23.368 | −18.230 | 31.662 | 1.00 | 28.01 | | S |
| ATOM | 347 | C | CYS G | 69 | 24.339 | −21.120 | 31.667 | 1.00 | 27.69 | | C |
| ATOM | 348 | O | CYS G | 69 | 23.967 | −21.533 | 30.570 | 1.00 | 27.41 | | O |
| ATOM | 349 | N | ASN G | 70 | 23.718 | −21.458 | 32.800 | 1.00 | 28.06 | | N |
| ATOM | 350 | CA | ASN G | 70 | 22.359 | −22.029 | 32.778 | 1.00 | 28.87 | | C |
| ATOM | 351 | CB | ASN G | 70 | 22.332 | −23.517 | 33.167 | 1.00 | 30.52 | | C |
| ATOM | 352 | CG | ASN G | 70 | 23.711 | −24.156 | 33.213 | 1.00 | 39.60 | | C |
| ATOM | 353 | OD1 | ASN G | 70 | 24.336 | −24.404 | 32.180 | 1.00 | 44.97 | | O |
| ATOM | 354 | ND2 | ASN G | 70 | 24.204 | −24.392 | 34.429 | 1.00 | 51.04 | | N |
| ATOM | 355 | C | ASN G | 70 | 21.297 | −21.261 | 33.577 | 1.00 | 26.97 | | C |
| ATOM | 356 | O | ASN G | 70 | 21.275 | −21.320 | 34.807 | 1.00 | 26.50 | | O |
| ATOM | 357 | N | GLY G | 71 | 20.320 | −20.703 | 32.866 | 1.00 | 25.46 | | N |
| ATOM | 358 | CA | GLY G | 71 | 19.231 | −19.956 | 33.496 | 1.00 | 23.67 | | C |
| ATOM | 359 | C | GLY G | 71 | 17.990 | −20.807 | 33.709 | 1.00 | 22.81 | | C |
| ATOM | 360 | O | GLY G | 71 | 18.004 | −22.008 | 33.436 | 1.00 | 22.79 | | O |
| ATOM | 361 | N | THR G | 72 | 16.924 | −20.195 | 34.225 | 1.00 | 21.93 | | N |
| ATOM | 362 | CA | THR G | 72 | 15.666 | −20.910 | 34.453 | 1.00 | 21.30 | | C |
| ATOM | 363 | CB | THR G | 72 | 14.737 | −20.162 | 35.441 | 1.00 | 21.12 | | C |
| ATOM | 364 | OG1 | THR G | 72 | 14.073 | −19.090 | 34.761 | 1.00 | 19.87 | | O |
| ATOM | 365 | CG2 | THR G | 72 | 15.532 | −19.609 | 36.619 | 1.00 | 20.76 | | C |
| ATOM | 366 | C | THR G | 72 | 14.923 | −21.169 | 33.139 | 1.00 | 21.50 | | C |
| ATOM | 367 | O | THR G | 72 | 14.471 | −20.236 | 32.474 | 1.00 | 21.85 | | O |
| ATOM | 368 | N | ASP G | 73 | 14.797 | −22.445 | 32.781 | 1.00 | 21.43 | | N |
| ATOM | 369 | CA | ASP G | 73 | 14.514 | −22.861 | 31.404 | 1.00 | 21.46 | | C |
| ATOM | 370 | CB | ASP G | 73 | 13.109 | −22.427 | 30.971 | 1.00 | 21.69 | | C |
| ATOM | 371 | CG | ASP G | 73 | 12.794 | −22.806 | 29.529 | 1.00 | 22.46 | | C |
| ATOM | 372 | OD1 | ASP G | 73 | 13.730 | −22.837 | 28.701 | 1.00 | 23.01 | | O |
| ATOM | 373 | OD2 | ASP G | 73 | 11.611 | −23.068 | 29.224 | 1.00 | 23.09 | | O |
| ATOM | 374 | C | ASP G | 73 | 15.561 | −22.365 | 30.406 | 1.00 | 20.96 | | C |
| ATOM | 375 | O | ASP G | 73 | 15.703 | −21.161 | 30.188 | 1.00 | 20.61 | | O |
| ATOM | 376 | N | ALA G | 74 | 16.237 | −23.305 | 29.748 | 1.00 | 20.42 | | N |
| ATOM | 377 | CA | ALA G | 74 | 17.299 | −22.967 | 28.802 | 1.00 | 20.38 | | C |
| ATOM | 378 | CB | ALA G | 74 | 18.658 | −23.365 | 29.363 | 1.00 | 20.11 | | C |
| ATOM | 379 | C | ALA G | 74 | 17.092 | −23.586 | 27.421 | 1.00 | 20.45 | | C |
| ATOM | 380 | O | ALA G | 74 | 18.059 | −23.849 | 26.707 | 1.00 | 20.46 | | O |
| ATOM | 381 | N | LYS G | 75 | 15.839 | −23.747 | 27.010 | 1.00 | 20.67 | | N |
| ATOM | 382 | CA | LYS G | 75 | 15.550 | −24.031 | 25.610 | 1.00 | 20.99 | | C |
| ATOM | 383 | CB | LYS G | 75 | 14.110 | −24.512 | 25.445 | 1.00 | 21.20 | | C |
| ATOM | 384 | CG | LYS G | 75 | 13.808 | −25.810 | 26.181 | 1.00 | 22.02 | | C |
| ATOM | 385 | CD | LYS G | 75 | 12.692 | −25.624 | 27.198 | 1.00 | 23.66 | | C |
| ATOM | 386 | CE | LYS G | 75 | 13.000 | −26.348 | 28.498 | 1.00 | 24.93 | | C |
| ATOM | 387 | NZ | LYS G | 75 | 12.517 | −25.588 | 29.683 | 1.00 | 25.55 | | N |
| ATOM | 388 | C | LYS G | 75 | 15.798 | −22.788 | 24.765 | 1.00 | 20.92 | | C |
| ATOM | 389 | O | LYS G | 75 | 16.187 | −22.880 | 23.600 | 1.00 | 20.92 | | O |
| ATOM | 390 | N | VAL G | 76 | 15.659 | −21.629 | 25.399 | 1.00 | 20.63 | | N |
| ATOM | 391 | CA | VAL G | 76 | 16.187 | −20.379 | 24.872 | 1.00 | 20.36 | | C |
| ATOM | 392 | CB | VAL G | 76 | 15.654 | −19.174 | 25.673 | 1.00 | 20.25 | | C |
| ATOM | 393 | CG1 | VAL G | 76 | 16.330 | −17.891 | 25.215 | 1.00 | 20.82 | | C |
| ATOM | 394 | CG2 | VAL G | 76 | 14.139 | −19.068 | 25.530 | 1.00 | 20.09 | | C |
| ATOM | 395 | C | VAL G | 76 | 17.717 | −20.356 | 24.872 | 1.00 | 20.29 | | C |
| ATOM | 396 | O | VAL G | 76 | 18.353 | −20.568 | 25.905 | 1.00 | 20.21 | | O |
| ATOM | 397 | N | LYS G | 77 | 18.294 | −20.039 | 23.716 | 1.00 | 20.17 | | N |
| ATOM | 398 | CA | LYS G | 77 | 19.745 | −20.018 | 23.547 | 1.00 | 20.17 | | C |
| ATOM | 399 | CB | LYS G | 77 | 20.267 | −21.419 | 23.194 | 1.00 | 20.62 | | C |
| ATOM | 400 | CG | LYS G | 77 | 20.894 | −22.180 | 24.367 | 1.00 | 23.31 | | C |
| ATOM | 401 | CD | LYS G | 77 | 20.940 | −23.684 | 24.105 | 1.00 | 26.74 | | C |
| ATOM | 402 | CE | LYS G | 77 | 21.242 | −24.468 | 25.376 | 1.00 | 28.32 | | C |
| ATOM | 403 | NZ | LYS G | 77 | 21.041 | −25.932 | 25.189 | 1.00 | 29.66 | | N |
| ATOM | 404 | C | LYS G | 77 | 20.159 | −19.010 | 22.468 | 1.00 | 19.45 | | C |
| ATOM | 405 | O | LYS G | 77 | 20.754 | −19.378 | 21.453 | 1.00 | 19.36 | | O |
| ATOM | 406 | N | LEU G | 78 | 19.862 | −17.736 | 22.710 | 1.00 | 18.70 | | N |
| ATOM | 407 | CA | LEU G | 78 | 19.857 | −16.728 | 21.656 | 1.00 | 17.98 | | C |
| ATOM | 408 | CB | LEU G | 78 | 19.534 | −15.357 | 22.246 | 1.00 | 17.72 | | C |
| ATOM | 409 | CG | LEU G | 78 | 18.293 | −15.357 | 23.142 | 1.00 | 17.51 | | C |
| ATOM | 410 | CD1 | LEU G | 78 | 18.117 | −14.023 | 23.860 | 1.00 | 17.41 | | C |
| ATOM | 411 | CD2 | LEU G | 78 | 17.045 | −15.734 | 22.349 | 1.00 | 17.62 | | C |
| ATOM | 412 | C | LEU G | 78 | 21.181 | −16.680 | 20.908 | 1.00 | 17.77 | | C |
| ATOM | 413 | O | LEU G | 78 | 21.209 | −16.672 | 19.678 | 1.00 | 17.89 | | O |
| ATOM | 414 | N | ILE G | 79 | 22.273 | −16.585 | 21.657 | 1.00 | 17.29 | | N |
| ATOM | 415 | CA | ILE G | 79 | 23.592 | −16.438 | 21.058 | 1.00 | 17.00 | | C |
| ATOM | 416 | CB | ILE G | 79 | 24.691 | −16.295 | 22.126 | 1.00 | 16.96 | | C |
| ATOM | 417 | CG1 | ILE G | 79 | 24.486 | −15.011 | 22.932 | 1.00 | 17.21 | | C |
| ATOM | 418 | CD1 | ILE G | 79 | 25.529 | −14.794 | 24.009 | 1.00 | 17.51 | | C |
| ATOM | 419 | CG2 | ILE G | 79 | 26.066 | −16.303 | 21.477 | 1.00 | 17.30 | | C |
| ATOM | 420 | C | ILE G | 79 | 23.923 | −17.616 | 20.147 | 1.00 | 16.83 | | C |
| ATOM | 421 | O | ILE G | 79 | 24.307 | −17.428 | 18.992 | 1.00 | 17.02 | | O |
| ATOM | 422 | N | LYS G | 80 | 23.678 | −18.870 | 20.478 | 1.00 | 16.55 | | N |
| ATOM | 423 | CA | LYS G | 80 | 24.058 | −19.851 | 19.437 | 1.00 | 16.38 | | C |
| ATOM | 424 | CB | LYS G | 80 | 23.872 | −21.278 | 19.959 | 1.00 | 16.73 | | C |
| ATOM | 425 | CG | LYS G | 80 | 24.274 | −22.360 | 18.971 | 1.00 | 17.99 | | C |
| ATOM | 426 | CD | LYS G | 80 | 25.741 | −22.245 | 18.590 | 1.00 | 20.71 | | C |
| ATOM | 427 | CE | LYS G | 80 | 26.170 | −23.396 | 17.697 | 1.00 | 22.92 | | C |
| ATOM | 428 | NZ | LYS G | 80 | 25.352 | −23.472 | 16.454 | 1.00 | 24.75 | | N |
| ATOM | 429 | C | LYS G | 80 | 23.334 | −19.690 | 18.060 | 1.00 | 15.79 | | C |
| ATOM | 430 | O | LYS G | 80 | 23.970 | −19.700 | 16.969 | 1.00 | 15.32 | | O |
| ATOM | 431 | N | GLN G | 81 | 22.019 | −19.489 | 18.112 | 1.00 | 15.48 | | N |
| ATOM | 432 | CA | GLN G | 81 | 21.192 | −19.385 | 16.905 | 1.00 | 15.33 | | C |
| ATOM | 433 | CB | GLN G | 81 | 19.711 | −19.320 | 17.287 | 1.00 | 15.70 | | C |
| ATOM | 434 | CG | GLN G | 81 | 19.269 | −20.415 | 18.242 | 1.00 | 17.88 | | C |
| ATOM | 435 | CD | GLN G | 81 | 17.848 | −20.225 | 18.734 | 1.00 | 20.46 | | C |
| ATOM | 436 | OE1 | GLN G | 81 | 17.116 | −19.367 | 18.241 | 1.00 | 21.59 | | O |
| ATOM | 437 | NE2 | GLN G | 81 | 17.451 | −21.028 | 19.716 | 1.00 | 20.26 | | N |
| ATOM | 438 | C | GLN G | 81 | 21.546 | −18.192 | 16.019 | 1.00 | 14.63 | | C |
| ATOM | 439 | O | GLN G | 81 | 21.582 | −18.295 | 14.780 | 1.00 | 14.60 | | O |
| ATOM | 440 | N | GLU G | 82 | 21.808 | −17.057 | 16.658 | 1.00 | 13.81 | | N |
| ATOM | 441 | CA | GLU G | 82 | 22.159 | −15.854 | 15.925 | 1.00 | 13.37 | | C |
| ATOM | 442 | CB | GLU G | 82 | 22.317 | −14.664 | 16.873 | 1.00 | 13.20 | | C |
| ATOM | 443 | CG | GLU G | 82 | 21.010 | −13.984 | 17.235 | 1.00 | 15.04 | | C |
| ATOM | 444 | CD | GLU G | 82 | 20.314 | −13.388 | 16.026 | 1.00 | 17.51 | | C |
| ATOM | 445 | OE1 | GLU G | 82 | 19.726 | −12.293 | 16.154 | 1.00 | 18.22 | | O |
| ATOM | 446 | OE2 | GLU G | 82 | 20.355 | −14.015 | 14.946 | 1.00 | 17.87 | | O |
| ATOM | 447 | C | GLU G | 82 | 23.450 | −16.101 | 15.169 | 1.00 | 12.99 | | C |
| ATOM | 448 | O | GLU G | 82 | 23.584 | −15.705 | 14.014 | 1.00 | 12.89 | | O |
| ATOM | 449 | N | LEU G | 83 | 24.393 | −16.778 | 15.815 | 1.00 | 12.89 | | N |
| ATOM | 450 | CA | LEU G | 83 | 25.665 | −17.066 | 15.174 | 1.00 | 13.08 | | C |
| ATOM | 451 | CB | LEU G | 83 | 26.613 | −17.761 | 16.151 | 1.00 | 13.26 | | C |
| ATOM | 452 | CG | LEU G | 83 | 26.970 | −16.978 | 17.415 | 1.00 | 14.18 | | C |
| ATOM | 453 | CD1 | LEU G | 83 | 27.933 | −17.774 | 18.282 | 1.00 | 15.11 | | C |
| ATOM | 454 | CD2 | LEU G | 83 | 27.557 | −15.620 | 17.059 | 1.00 | 14.79 | | C |
| ATOM | 455 | C | LEU G | 83 | 25.444 | −17.940 | 13.949 | 1.00 | 13.18 | | C |
| ATOM | 456 | O | LEU G | 83 | 26.007 | −17.673 | 12.876 | 1.00 | 13.05 | | O |
| ATOM | 457 | N | ASP G | 84 | 24.588 | −18.953 | 14.074 | 1.00 | 13.56 | | N |
| ATOM | 458 | CA | ASP G | 84 | 24.346 | −19.788 | 12.891 | 1.00 | 14.15 | | C |
| ATOM | 459 | CB | ASP G | 84 | 23.439 | −20.973 | 13.232 | 1.00 | 14.80 | | C |
| ATOM | 460 | CG | ASP G | 84 | 24.004 | −21.841 | 14.338 | 1.00 | 18.26 | | C |
| ATOM | 461 | OD1 | ASP G | 84 | 24.605 | −22.891 | 14.026 | 1.00 | 21.11 | | O |
| ATOM | 462 | OD2 | ASP G | 84 | 23.837 | −21.480 | 15.520 | 1.00 | 22.12 | | O |
| ATOM | 463 | C | ASP G | 84 | 23.737 | −18.979 | 11.731 | 1.00 | 13.56 | | C |
| ATOM | 464 | O | ASP G | 84 | 24.147 | −19.111 | 10.549 | 1.00 | 13.70 | | O |
| ATOM | 465 | N | LYS G | 85 | 22.780 | −18.115 | 12.068 | 1.00 | 12.87 | | N |
| ATOM | 466 | CA | LYS G | 85 | 22.128 | −17.318 | 11.033 | 1.00 | 12.31 | | C |
| ATOM | 467 | CB | LYS G | 85 | 20.987 | −16.487 | 11.619 | 1.00 | 12.41 | | C |
| ATOM | 468 | CG | LYS G | 85 | 20.087 | −15.857 | 10.568 | 1.00 | 12.26 | | C |
| ATOM | 469 | CD | LYS G | 85 | 19.083 | −14.900 | 11.189 | 1.00 | 12.72 | | C |
| ATOM | 470 | CE | LYS G | 85 | 19.772 | −13.667 | 11.750 | 1.00 | 14.40 | | C |
| ATOM | 471 | NZ | LYS G | 85 | 18.789 | −12.676 | 12.268 | 1.00 | 16.45 | | N |
| ATOM | 472 | C | LYS G | 85 | 23.147 | −16.409 | 10.353 | 1.00 | 11.85 | | C |
| ATOM | 473 | O | LYS G | 85 | 23.143 | −16.238 | 9.125 | 1.00 | 11.66 | | O |
| ATOM | 474 | N | TYR G | 86 | 24.031 | −15.839 | 11.165 | 1.00 | 11.32 | | N |
| ATOM | 475 | CA | TYR G | 86 | 25.051 | −14.936 | 10.665 | 1.00 | 11.07 | | C |
| ATOM | 476 | CB | TYR G | 86 | 25.863 | −14.349 | 11.820 | 1.00 | 11.12 | | C |
| ATOM | 477 | CG | TYR G | 86 | 27.216 | −13.824 | 11.399 | 1.00 | 11.30 | | C |
| ATOM | 478 | CD1 | TYR G | 86 | 27.349 | −12.561 | 10.838 | 1.00 | 12.16 | | C |
| ATOM | 479 | CE1 | TYR G | 86 | 28.584 | −12.080 | 10.449 | 1.00 | 12.39 | | C |
| ATOM | 480 | CZ | TYR G | 86 | 29.705 | −12.864 | 10.619 | 1.00 | 12.62 | | C |
| ATOM | 481 | OH | TYR G | 86 | 30.938 | −12.388 | 10.233 | 1.00 | 12.72 | | O |
| ATOM | 482 | CE2 | TYR G | 86 | 29.599 | −14.121 | 11.172 | 1.00 | 11.38 | | C |
| ATOM | 483 | CD2 | TYR G | 86 | 28.360 | −14.595 | 11.558 | 1.00 | 11.05 | | C |
| ATOM | 484 | C | TYR G | 86 | 25.971 | −15.667 | 9.708 | 1.00 | 10.75 | | C |
| ATOM | 485 | O | TYR G | 86 | 26.320 | −15.140 | 8.656 | 1.00 | 10.45 | | O |
| ATOM | 486 | N | LYS G | 87 | 26.343 | −16.894 | 10.053 | 1.00 | 10.66 | | N |
| ATOM | 487 | CA | LYS G | 87 | 27.220 | −17.656 | 9.174 | 1.00 | 11.03 | | C |
| ATOM | 488 | CB | LYS G | 87 | 27.588 | −18.996 | 9.813 | 1.00 | 11.36 | | C |
| ATOM | 489 | CG | LYS G | 87 | 28.300 | −18.874 | 11.149 | 1.00 | 12.79 | | C |
| ATOM | 490 | CD | LYS G | 87 | 28.627 | −20.242 | 11.722 | 1.00 | 14.76 | | C |
| ATOM | 491 | CE | LYS G | 87 | 29.336 | −20.123 | 13.060 | 1.00 | 15.29 | | C |
| ATOM | 492 | NZ | LYS G | 87 | 29.664 | −21.460 | 13.626 | 1.00 | 16.27 | | N |
| ATOM | 493 | C | LYS G | 87 | 26.541 | −17.891 | 7.826 | 1.00 | 10.83 | | C |
| ATOM | 494 | O | LYS G | 87 | 27.163 | −17.715 | 6.758 | 1.00 | 11.11 | | O |
| ATOM | 495 | N | ASN G | 88 | 25.256 | −18.243 | 7.858 | 1.00 | 10.52 | | N |
| ATOM | 496 | CA | ASN G | 88 | 24.567 | −18.464 | 6.584 | 1.00 | 10.21 | | C |
| ATOM | 497 | CB | ASN G | 88 | 23.149 | −18.989 | 6.821 | 1.00 | 10.61 | | C |
| ATOM | 498 | CG | ASN G | 88 | 22.406 | −19.271 | 5.529 | 1.00 | 11.81 | | C |
| ATOM | 499 | OD1 | ASN G | 88 | 22.998 | −19.285 | 4.450 | 1.00 | 12.97 | | O |
| ATOM | 500 | ND2 | ASN G | 88 | 21.101 | −19.496 | 5.633 | 1.00 | 13.82 | | N |
| ATOM | 501 | C | ASN G | 88 | 24.528 | −17.187 | 5.730 | 1.00 | 9.42 | | C |
| ATOM | 502 | O | ASN G | 88 | 24.780 | −17.206 | 4.500 | 1.00 | 8.94 | | O |
| ATOM | 503 | N | ALA G | 89 | 24.251 | −16.068 | 6.395 | 1.00 | 8.77 | | N |
| ATOM | 504 | CA | ALA G | 89 | 24.164 | −14.796 | 5.693 | 1.00 | 8.21 | | C |
| ATOM | 505 | CB | ALA G | 89 | 23.734 | −13.693 | 6.647 | 1.00 | 7.99 | | C |
| ATOM | 506 | C | ALA G | 89 | 25.510 | −14.465 | 5.063 | 1.00 | 7.78 | | C |
| ATOM | 507 | O | ALA G | 89 | 25.586 | −13.988 | 3.927 | 1.00 | 7.88 | | O |
| ATOM | 508 | N | VAL G | 90 | 26.574 | −14.738 | 5.809 | 1.00 | 7.01 | | N |
| ATOM | 509 | CA | VAL G | 90 | 27.921 | −14.464 | 5.344 | 1.00 | 6.26 | | C |
| ATOM | 510 | CB | VAL G | 90 | 28.965 | −14.771 | 6.433 | 1.00 | 5.91 | | C |
| ATOM | 511 | CG1 | VAL G | 90 | 30.361 | −14.414 | 5.945 | 1.00 | 5.92 | | C |
| ATOM | 512 | CG2 | VAL G | 90 | 28.631 | −14.020 | 7.714 | 1.00 | 5.26 | | C |
| ATOM | 513 | C | VAL G | 90 | 28.240 | −15.284 | 4.104 | 1.00 | 6.25 | | C |
| ATOM | 514 | O | VAL G | 90 | 28.798 | −14.758 | 3.144 | 1.00 | 6.32 | | O |
| ATOM | 515 | N | THR G | 91 | 27.858 | −16.558 | 4.098 | 1.00 | 6.24 | | N |
| ATOM | 516 | CA | THR G | 91 | 28.129 | −17.376 | 2.915 | 1.00 | 6.68 | | C |
| ATOM | 517 | CB | THR G | 91 | 27.705 | −18.840 | 3.129 | 1.00 | 6.62 | | C |
| ATOM | 518 | OG1 | THR G | 91 | 28.438 | −19.397 | 4.227 | 1.00 | 7.19 | | O |
| ATOM | 519 | CG2 | THR G | 91 | 27.971 | −19.661 | 1.876 | 1.00 | 7.27 | | C |
| ATOM | 520 | C | THR G | 91 | 27.392 | −16.808 | 1.698 | 1.00 | 7.02 | | C |
| ATOM | 521 | O | THR G | 91 | 27.953 | −16.709 | 0.569 | 1.00 | 7.16 | | O |
| ATOM | 522 | N | GLU G | 92 | 26.143 | −16.402 | 1.925 | 1.00 | 7.31 | | N |
| ATOM | 523 | CA | GLU G | 92 | 25.382 | −15.849 | 0.812 | 1.00 | 7.81 | | C |
| ATOM | 524 | CB | GLU G | 92 | 23.942 | −15.547 | 1.231 | 1.00 | 8.23 | | C |
| ATOM | 525 | CG | GLU G | 92 | 23.030 | −16.761 | 1.222 | 1.00 | 10.91 | | C |
| ATOM | 526 | CD | GLU G | 92 | 22.895 | −17.372 | −0.161 | 1.00 | 14.95 | | C |
| ATOM | 527 | OE1 | GLU G | 92 | 21.801 | −17.878 | −0.489 | 1.00 | 16.14 | | O |
| ATOM | 528 | OE2 | GLU G | 92 | 23.884 | −17.346 | −0.923 | 1.00 | 16.53 | | O |
| ATOM | 529 | C | GLU G | 92 | 26.053 | −14.587 | 0.267 | 1.00 | 7.47 | | C |
| ATOM | 530 | O | GLU G | 92 | 26.210 | −14.432 | −0.947 | 1.00 | 7.32 | | O |
| ATOM | 531 | N | LEU G | 93 | 26.501 | −13.717 | 1.167 | 1.00 | 7.30 | | N |
| ATOM | 532 | CA | LEU G | 93 | 27.140 | −12.467 | 0.759 | 1.00 | 7.47 | | C |
| ATOM | 533 | CB | LEU G | 93 | 27.442 | −11.589 | 1.973 | 1.00 | 7.15 | | C |
| ATOM | 534 | CG | LEU G | 93 | 26.217 | −11.021 | 2.692 | 1.00 | 7.20 | | C |
| ATOM | 535 | CD1 | LEU G | 93 | 26.638 | −10.123 | 3.845 | 1.00 | 7.93 | | C |
| ATOM | 536 | CD2 | LEU G | 93 | 25.325 | −10.266 | 1.716 | 1.00 | 6.91 | | C |
| ATOM | 537 | C | LEU G | 93 | 28.414 | −12.761 | −0.020 | 1.00 | 7.89 | | C |
| ATOM | 538 | O | LEU G | 93 | 28.746 | −12.083 | −0.991 | 1.00 | 7.94 | | O |
| ATOM | 539 | N | GLN G | 94 | 29.116 | −13.789 | 0.434 | 1.00 | 8.31 | | N |
| ATOM | 540 | CA | GLN G | 94 | 30.361 | −14.248 | −0.160 | 1.00 | 9.05 | | C |
| ATOM | 541 | CB | GLN G | 94 | 30.985 | −15.349 | 0.701 | 1.00 | 9.18 | | C |
| ATOM | 542 | CG | GLN G | 94 | 31.324 | −14.908 | 2.114 | 1.00 | 10.79 | | C |
| ATOM | 543 | CD | GLN G | 94 | 31.993 | −16.003 | 2.920 | 1.00 | 13.82 | | C |
| ATOM | 544 | OE1 | GLN G | 94 | 32.210 | −17.109 | 2.427 | 1.00 | 14.98 | | O |
| ATOM | 545 | NE2 | GLN G | 94 | 32.324 | −15.698 | 4.170 | 1.00 | 15.25 | | N |
| ATOM | 546 | C | GLN G | 94 | 30.220 | −14.731 | −1.603 | 1.00 | 9.25 | | C |
| ATOM | 547 | O | GLN G | 94 | 31.124 | −14.514 | −2.410 | 1.00 | 9.43 | | O |
| ATOM | 548 | N | LEU G | 95 | 29.114 | −15.393 | −1.941 | 1.00 | 9.55 | | N |
| ATOM | 549 | CA | LEU G | 95 | 29.003 | −15.935 | −3.318 | 1.00 | 10.17 | | C |
| ATOM | 550 | CB | LEU G | 95 | 27.752 | −16.811 | −3.437 | 1.00 | 9.79 | | C |
| ATOM | 551 | CG | LEU G | 95 | 27.700 | −18.053 | −2.545 | 1.00 | 8.86 | | C |
| ATOM | 552 | CD1 | LEU G | 95 | 26.403 | −18.817 | −2.764 | 1.00 | 8.31 | | C |
| ATOM | 553 | CD2 | LEU G | 95 | 28.904 | −18.947 | −2.798 | 1.00 | 7.62 | | C |
| ATOM | 554 | C | LEU G | 95 | 29.057 | −14.945 | −4.528 | 1.00 | 11.22 | | C |
| ATOM | 555 | O | LEU G | 95 | 29.643 | −15.248 | −5.567 | 1.00 | 11.55 | | O |
| ATOM | 556 | N | LEU G | 96 | 28.430 | −13.786 | −4.361 | 1.00 | 12.40 | | N |
| ATOM | 557 | CA | LEU G | 96 | 28.142 | −12.747 | −5.335 | 1.00 | 13.66 | | C |
| ATOM | 558 | CB | LEU G | 96 | 27.471 | −11.548 | −4.668 | 1.00 | 13.38 | | C |
| ATOM | 559 | CG | LEU G | 96 | 26.008 | −11.779 | −4.288 | 1.00 | 13.32 | | C |
| ATOM | 560 | CD1 | LEU G | 96 | 25.321 | −10.462 | −3.975 | 1.00 | 12.84 | | C |
| ATOM | 561 | CD2 | LEU G | 96 | 25.278 | −12.519 | −5.397 | 1.00 | 13.38 | | C |
| ATOM | 562 | C | LEU G | 96 | 29.392 | −12.314 | −6.094 | 1.00 | 14.97 | | C |
| ATOM | 563 | O | LEU G | 96 | 29.293 | −11.741 | −7.178 | 1.00 | 15.21 | | O |
| ATOM | 564 | N | MET G | 97 | 30.564 | −12.678 | −5.576 | 1.00 | 16.74 | | N |
| ATOM | 565 | CA | MET G | 97 | 31.799 | −11.956 | −5.894 | 1.00 | 18.96 | | C |
| ATOM | 566 | CB | MET G | 97 | 32.828 | −12.102 | −4.766 | 1.00 | 19.12 | | C |
| ATOM | 567 | CG | MET G | 97 | 32.353 | −11.577 | −3.413 | 1.00 | 21.87 | | C |
| ATOM | 568 | SD | MET G | 97 | 32.586 | −9.794 | −3.210 | 1.00 | 27.46 | | S |
| ATOM | 569 | CE | MET G | 97 | 32.408 | −9.214 | −4.899 | 1.00 | 24.25 | | C |
| ATOM | 570 | C | MET G | 97 | 32.408 | −12.377 | −7.233 | 1.00 | 20.09 | | C |
| ATOM | 571 | O | MET G | 97 | 33.064 | −11.581 | −7.905 | 1.00 | 20.58 | | O |
| ATOM | 572 | N | GLN G | 98 | 32.151 | −13.618 | −7.634 | 1.00 | 21.33 | | N |
| ATOM | 573 | CA | GLN G | 98 | 32.735 | −14.171 | −8.853 | 1.00 | 22.21 | | C |
| ATOM | 574 | CB | GLN G | 98 | 33.777 | −15.243 | −8.513 | 1.00 | 22.53 | | C |
| ATOM | 575 | CG | GLN G | 98 | 34.695 | −14.883 | −7.347 | 1.00 | 23.85 | | C |
| ATOM | 576 | CD | GLN G | 98 | 34.172 | −15.376 | −6.008 | 1.00 | 25.21 | | C |
| ATOM | 577 | OE1 | GLN G | 98 | 33.109 | −14.957 | −5.549 | 1.00 | 24.47 | | O |
| ATOM | 578 | NE2 | GLN G | 98 | 34.939 | −16.243 | −5.358 | 1.00 | 24.85 | | N |
| ATOM | 579 | C | GLN G | 98 | 31.660 | −14.756 | −9.766 | 1.00 | 22.15 | | C |
| ATOM | 580 | O | GLN G | 98 | 30.475 | −14.451 | −9.624 | 1.00 | 21.77 | | O |
| ATOM | 581 | N | PHE G | 137 | 36.645 | −13.585 | −16.969 | 1.00 | 33.25 | | N |
| ATOM | 582 | CA | PHE G | 137 | 35.365 | −12.919 | −16.764 | 1.00 | 36.79 | | C |
| ATOM | 583 | CB | PHE G | 137 | 35.255 | −11.672 | −17.642 | 1.00 | 20.00 | | C |
| ATOM | 584 | CG | PHE G | 137 | 36.265 | −10.611 | −17.319 | 1.00 | 20.00 | | C |
| ATOM | 585 | CD1 | PHE G | 137 | 36.015 | −9.677 | −16.331 | 1.00 | 20.00 | | C |
| ATOM | 586 | CE1 | PHE G | 137 | 36.932 | −8.682 | −16.050 | 1.00 | 20.00 | | C |
| ATOM | 587 | CZ | PHE G | 137 | 38.108 | −8.607 | −16.766 | 1.00 | 20.00 | | C |
| ATOM | 588 | CE2 | PHE G | 137 | 38.362 | −9.523 | −17.764 | 1.00 | 20.00 | | C |
| ATOM | 589 | CD2 | PHE G | 137 | 37.439 | −10.510 | −18.045 | 1.00 | 20.00 | | C |
| ATOM | 590 | C | PHE G | 137 | 34.195 | −13.850 | −17.063 | 1.00 | 48.73 | | C |
| ATOM | 591 | O | PHE G | 137 | 33.039 | −13.434 | −16.993 | 1.00 | 49.28 | | O |
| ATOM | 592 | N | LEU G | 138 | 34.498 | −15.040 | −17.571 | 1.00 | 42.04 | | N |
| ATOM | 593 | CA | LEU G | 138 | 33.471 | −16.059 | −17.783 | 1.00 | 44.26 | | C |
| ATOM | 594 | CB | LEU G | 138 | 34.037 | −17.258 | −18.553 | 1.00 | 20.00 | | C |
| ATOM | 595 | CG | LEU G | 138 | 34.341 | −17.114 | −20.050 | 1.00 | 20.00 | | C |
| ATOM | 596 | CD1 | LEU G | 138 | 34.975 | −18.387 | −20.605 | 1.00 | 20.00 | | C |
| ATOM | 597 | CD2 | LEU G | 138 | 33.095 | −16.756 | −20.843 | 1.00 | 20.00 | | C |
| ATOM | 598 | C | LEU G | 138 | 32.896 | −16.527 | −16.451 | 1.00 | 43.68 | | C |
| ATOM | 599 | O | LEU G | 138 | 31.839 | −17.157 | −16.406 | 1.00 | 50.27 | | O |
| ATOM | 600 | N | GLY G | 139 | 33.614 | −16.234 | −15.370 | 1.00 | 38.56 | | N |
| ATOM | 601 | CA | GLY G | 139 | 33.145 | −16.544 | −14.022 | 1.00 | 47.99 | | C |
| ATOM | 602 | C | GLY G | 139 | 31.826 | −15.881 | −13.662 | 1.00 | 44.53 | | C |
| ATOM | 603 | O | GLY G | 139 | 31.082 | −16.383 | −12.819 | 1.00 | 32.21 | | O |
| ATOM | 604 | N | PHE G | 140 | 31.510 | −14.778 | −14.337 | 1.00 | 46.33 | | N |
| ATOM | 605 | CA | PHE G | 140 | 30.319 | −13.992 | −14.021 | 1.00 | 42.95 | | C |
| ATOM | 606 | CB | PHE G | 140 | 30.530 | −12.516 | −14.386 | 1.00 | 20.00 | | C |
| ATOM | 607 | CG | PHE G | 140 | 31.590 | −11.827 | −13.564 | 1.00 | 20.00 | | C |
| ATOM | 608 | CD1 | PHE G | 140 | 31.295 | −11.318 | −12.310 | 1.00 | 20.00 | | C |
| ATOM | 609 | CE1 | PHE G | 140 | 32.272 | −10.683 | −11.553 | 1.00 | 20.00 | | C |
| ATOM | 610 | CZ | PHE G | 140 | 33.540 | −10.511 | −12.066 | 1.00 | 20.00 | | C |
| ATOM | 611 | CE2 | PHE G | 140 | 33.832 | −10.970 | −13.334 | 1.00 | 20.00 | | C |
| ATOM | 612 | CD2 | PHE G | 140 | 32.851 | −11.591 | −14.090 | 1.00 | 20.00 | | C |
| ATOM | 613 | C | PHE G | 140 | 29.077 | −14.525 | −14.731 | 1.00 | 38.13 | | C |
| ATOM | 614 | O | PHE G | 140 | 27.970 | −14.040 | −14.504 | 1.00 | 57.33 | | O |
| ATOM | 615 | N | LEU G | 141 | 29.271 | −15.480 | −15.634 | 1.00 | 35.20 | | N |
| ATOM | 616 | CA | LEU G | 141 | 28.173 | −15.996 | −16.446 | 1.00 | 27.01 | | C |
| ATOM | 617 | CB | LEU G | 141 | 26.983 | −16.392 | −15.560 | 1.00 | 20.00 | | C |
| ATOM | 618 | CG | LEU G | 141 | 27.185 | −17.555 | −14.580 | 1.00 | 20.00 | | C |
| ATOM | 619 | CD1 | LEU G | 141 | 25.930 | −17.779 | −13.747 | 1.00 | 20.00 | | C |
| ATOM | 620 | CD2 | LEU G | 141 | 27.588 | −18.835 | −15.306 | 1.00 | 20.00 | | C |
| ATOM | 621 | C | LEU G | 141 | 27.750 | −14.978 | −17.504 | 1.00 | 35.79 | | C |
| ATOM | 622 | O | LEU G | 141 | 28.543 | −14.613 | −18.370 | 1.00 | 46.92 | | O |
| ATOM | 623 | N | LEU G | 142 | 26.509 | −14.508 | −17.424 | 1.00 | 38.67 | | N |
| ATOM | 624 | CA | LEU G | 142 | 26.075 | −13.372 | −18.231 | 1.00 | 33.84 | | C |
| ATOM | 625 | CB | LEU G | 142 | 24.737 | −13.667 | −18.914 | 1.00 | 20.00 | | C |
| ATOM | 626 | CG | LEU G | 142 | 24.720 | −14.676 | −20.063 | 1.00 | 20.00 | | C |
| ATOM | 627 | CD1 | LEU G | 142 | 23.339 | −14.707 | −20.705 | 1.00 | 20.00 | | C |
| ATOM | 628 | CD2 | LEU G | 142 | 25.773 | −14.324 | −21.101 | 1.00 | 20.00 | | C |
| ATOM | 629 | C | LEU G | 142 | 25.959 | −12.123 | −17.368 | 1.00 | 36.89 | | C |
| ATOM | 630 | O | LEU G | 142 | 25.257 | −12.119 | −16.358 | 1.00 | 45.42 | | O |
| ATOM | 631 | N | GLY G | 143 | 26.763 | −11.115 | −17.688 | 1.00 | 26.84 | | N |
| ATOM | 632 | CA | GLY G | 143 | 26.817 | −9.895 | −16.892 | 1.00 | 27.41 | | C |
| ATOM | 633 | C | GLY G | 143 | 26.590 | −8.645 | −17.722 | 1.00 | 29.08 | | C |
| ATOM | 634 | O | GLY G | 143 | 26.918 | −8.603 | −18.908 | 1.00 | 40.24 | | O |
| ATOM | 635 | N | VAL G | 144 | 26.026 | −7.622 | −17.092 | 1.00 | 30.17 | | N |
| ATOM | 636 | CA | VAL G | 144 | 25.849 | −6.331 | −17.739 | 1.00 | 34.15 | | C |
| ATOM | 637 | CB | VAL G | 144 | 24.768 | −5.496 | −17.035 | 1.00 | 20.00 | | C |
| ATOM | 638 | CG1 | VAL G | 144 | 24.730 | −4.081 | −17.599 | 1.00 | 20.00 | | C |
| ATOM | 639 | CG2 | VAL G | 144 | 23.414 | −6.166 | −17.170 | 1.00 | 20.00 | | C |
| ATOM | 640 | C | VAL G | 144 | 27.152 | −5.543 | −17.760 | 1.00 | 36.67 | | C |
| ATOM | 641 | O | VAL G | 144 | 28.104 | −5.880 | −17.054 | 1.00 | 42.49 | | O |
| ATOM | 642 | N | GLY G | 145 | 27.185 | −4.487 | −18.567 | 1.00 | 47.09 | | N |
| ATOM | 643 | CA | GLY G | 145 | 28.309 | −3.563 | −18.568 | 1.00 | 44.51 | | C |
| ATOM | 644 | C | GLY G | 145 | 28.683 | −3.184 | −17.151 | 1.00 | 44.94 | | C |
| ATOM | 645 | O | GLY G | 145 | 29.809 | −3.424 | −16.714 | 1.00 | 40.58 | | O |
| ATOM | 646 | N | SER G | 146 | 27.732 | −2.662 | −16.387 | 1.00 | 42.06 | | N |
| ATOM | 647 | CA | SER G | 146 | 28.033 | −2.190 | −15.035 | 1.00 | 47.39 | | C |
| ATOM | 648 | CB | SER G | 146 | 26.797 | −1.534 | −14.414 | 1.00 | 20.00 | | C |
| ATOM | 649 | OG | SER G | 146 | 25.719 | −2.450 | −14.333 | 1.00 | 20.00 | | O |
| ATOM | 650 | C | SER G | 146 | 28.570 | −3.280 | −14.101 | 1.00 | 51.48 | | C |
| ATOM | 651 | O | SER G | 146 | 29.491 | −3.034 | −13.317 | 1.00 | 47.45 | | O |
| ATOM | 652 | N | ALA G | 147 | 28.004 | −4.480 | −14.183 | 1.00 | 39.51 | | N |
| ATOM | 653 | CA | ALA G | 147 | 28.430 | −5.576 | −13.316 | 1.00 | 43.38 | | C |
| ATOM | 654 | CB | ALA G | 147 | 27.531 | −6.788 | −13.513 | 1.00 | 20.00 | | C |
| ATOM | 655 | C | ALA G | 147 | 29.894 | −5.954 | −13.541 | 1.00 | 47.39 | | C |
| ATOM | 656 | O | ALA G | 147 | 30.635 | −6.211 | −12.587 | 1.00 | 50.18 | | O |
| ATOM | 657 | N | ILE G | 148 | 30.309 | −5.983 | −14.804 | 1.00 | 44.67 | | N |
| ATOM | 658 | CA | ILE G | 148 | 31.690 | −6.311 | −15.140 | 1.00 | 43.56 | | C |
| ATOM | 659 | CB | ILE G | 148 | 31.898 | −6.390 | −16.663 | 1.00 | 20.00 | | C |
| ATOM | 660 | CG1 | ILE G | 148 | 30.998 | −7.469 | −17.269 | 1.00 | 20.00 | | C |
| ATOM | 661 | CD1 | ILE G | 148 | 31.142 | −7.612 | −18.768 | 1.00 | 20.00 | | C |
| ATOM | 662 | CG2 | ILE G | 148 | 33.358 | −6.663 | −16.987 | 1.00 | 20.00 | | C |
| ATOM | 663 | C | ILE G | 148 | 32.633 | −5.265 | −14.558 | 1.00 | 51.18 | | C |
| ATOM | 664 | O | ILE G | 148 | 33.699 | −5.592 | −14.031 | 1.00 | 39.17 | | O |
| ATOM | 665 | N | ALA G | 149 | 32.226 | −4.003 | −14.651 | 1.00 | 42.17 | | N |
| ATOM | 666 | CA | ALA G | 149 | 33.020 | −2.906 | −14.116 | 1.00 | 36.76 | | C |
| ATOM | 667 | CB | ALA G | 149 | 32.386 | −1.571 | −14.466 | 1.00 | 20.00 | | C |
| ATOM | 668 | C | ALA G | 149 | 33.150 | −3.058 | −12.607 | 1.00 | 45.19 | | C |
| ATOM | 669 | O | ALA G | 149 | 34.222 | −2.843 | −12.041 | 1.00 | 47.83 | | O |
| ATOM | 670 | N | SER G | 150 | 32.054 | −3.443 | −11.961 | 1.00 | 43.64 | | N |
| ATOM | 671 | CA | SER G | 150 | 32.062 | −3.643 | −10.518 | 1.00 | 43.46 | | C |
| ATOM | 672 | CB | SER G | 150 | 30.657 | −3.968 | −10.010 | 1.00 | 20.00 | | C |
| ATOM | 673 | OG | SER G | 150 | 30.165 | −5.160 | −10.596 | 1.00 | 20.00 | | O |
| ATOM | 674 | C | SER G | 150 | 33.030 | −4.762 | −10.146 | 1.00 | 44.80 | | C |
| ATOM | 675 | O | SER G | 150 | 33.774 | −4.653 | −9.170 | 1.00 | 41.55 | | O |
| ATOM | 676 | N | GLY G | 151 | 33.025 | −5.833 | −10.935 | 1.00 | 27.01 | | N |
| ATOM | 677 | CA | GLY G | 151 | 33.927 | −6.946 | −10.697 | 1.00 | 35.21 | | C |
| ATOM | 678 | C | GLY G | 151 | 35.376 | −6.519 | −10.839 | 1.00 | 42.40 | | C |
| ATOM | 679 | O | GLY G | 151 | 36.246 | −6.924 | −10.055 | 1.00 | 45.11 | | O |
| ATOM | 680 | N | VAL G | 152 | 35.638 | −5.691 | −11.846 | 1.00 | 40.20 | | N |
| ATOM | 681 | CA | VAL G | 152 | 36.983 | −5.185 | −12.076 | 1.00 | 46.73 | | C |
| ATOM | 682 | CB | VAL G | 152 | 37.056 | −4.330 | −13.354 | 1.00 | 20.00 | | C |
| ATOM | 683 | CG1 | VAL G | 152 | 38.472 | −3.816 | −13.568 | 1.00 | 20.00 | | C |
| ATOM | 684 | CG2 | VAL G | 152 | 36.587 | −5.134 | −14.557 | 1.00 | 20.00 | | C |
| ATOM | 685 | C | VAL G | 152 | 37.424 | −4.345 | −10.885 | 1.00 | 48.62 | | C |
| ATOM | 686 | O | VAL G | 152 | 38.565 | −4.438 | −10.432 | 1.00 | 49.32 | | O |
| ATOM | 687 | N | ALA G | 153 | 36.502 | −3.537 | −10.370 | 1.00 | 35.09 | | N |
| ATOM | 688 | CA | ALA G | 153 | 36.788 | −2.690 | −9.220 | 1.00 | 42.37 | | C |
| ATOM | 689 | CB | ALA G | 153 | 35.604 | −1.787 | −8.918 | 1.00 | 20.00 | | C |
| ATOM | 690 | C | ALA G | 153 | 37.120 | −3.557 | −8.014 | 1.00 | 37.79 | | C |
| ATOM | 691 | O | ALA G | 153 | 38.029 | −3.247 | −7.243 | 1.00 | 39.25 | | O |
| ATOM | 692 | N | VAL G | 154 | 36.378 | −4.648 | −7.859 | 1.00 | 38.88 | | N |
| ATOM | 693 | CA | VAL G | 154 | 36.610 | −5.579 | −6.763 | 1.00 | 35.53 | | C |
| ATOM | 694 | CB | VAL G | 154 | 35.549 | −6.695 | −6.737 | 1.00 | 20.00 | | C |
| ATOM | 695 | CG1 | VAL G | 154 | 35.814 | −7.651 | −5.584 | 1.00 | 20.00 | | C |
| ATOM | 696 | CG2 | VAL G | 154 | 34.154 | −6.098 | −6.634 | 1.00 | 20.00 | | C |
| ATOM | 697 | C | VAL G | 154 | 37.996 | −6.209 | −6.863 | 1.00 | 41.00 | | C |
| ATOM | 698 | O | VAL G | 154 | 38.690 | −6.353 | −5.856 | 1.00 | 50.08 | | O |
| ATOM | 699 | N | SER G | 155 | 38.403 | −6.579 | −8.075 | 1.00 | 37.82 | | N |
| ATOM | 700 | CA | SER G | 155 | 39.733 | −7.158 | −8.271 | 1.00 | 44.68 | | C |
| ATOM | 701 | CB | SER G | 155 | 39.906 | −7.630 | −9.715 | 1.00 | 20.00 | | C |
| ATOM | 702 | OG | SER G | 155 | 39.757 | −6.554 | −10.625 | 1.00 | 20.00 | | O |
| ATOM | 703 | C | SER G | 155 | 40.817 | −6.140 | −7.914 | 1.00 | 41.82 | | C |
| ATOM | 704 | O | SER G | 155 | 41.834 | −6.468 | −7.304 | 1.00 | 49.37 | | O |
| ATOM | 705 | N | LYS G | 156 | 40.566 | −4.900 | −8.314 | 1.00 | 36.99 | | N |
| ATOM | 706 | CA | LYS G | 156 | 41.440 | −3.756 | −8.097 | 1.00 | 35.11 | | C |
| ATOM | 707 | CB | LYS G | 156 | 40.989 | −2.562 | −8.943 | 1.00 | 20.00 | | C |
| ATOM | 708 | CG | LYS G | 156 | 41.146 | −2.767 | −10.443 | 1.00 | 20.00 | | C |
| ATOM | 709 | CD | LYS G | 156 | 40.541 | −1.606 | −11.227 | 1.00 | 20.00 | | C |
| ATOM | 710 | CE | LYS G | 156 | 40.906 | −1.679 | −12.708 | 1.00 | 20.00 | | C |
| ATOM | 711 | NZ | LYS G | 156 | 40.298 | −0.569 | −13.497 | 1.00 | 20.00 | | N |
| ATOM | 712 | C | LYS G | 156 | 41.489 | −3.378 | −6.620 | 1.00 | 35.98 | | C |
| ATOM | 713 | O | LYS G | 156 | 42.501 | −2.874 | −6.136 | 1.00 | 42.31 | | O |
| ATOM | 714 | N | VAL G | 157 | 40.433 | −3.719 | −5.886 | 1.00 | 37.88 | | N |
| ATOM | 715 | CA | VAL G | 157 | 40.341 | −3.361 | −4.475 | 1.00 | 50.94 | | C |
| ATOM | 716 | CB | VAL G | 157 | 38.894 | −3.057 | −4.061 | 1.00 | 20.00 | | C |
| ATOM | 717 | CG1 | VAL G | 157 | 38.799 | −2.928 | −2.551 | 1.00 | 20.00 | | C |
| ATOM | 718 | CG2 | VAL G | 157 | 38.403 | −1.790 | −4.745 | 1.00 | 20.00 | | C |
| ATOM | 719 | C | VAL G | 157 | 40.882 | −4.474 | −3.591 | 1.00 | 47.82 | | C |
| ATOM | 720 | O | VAL G | 157 | 41.021 | −4.309 | −2.379 | 1.00 | 56.87 | | O |
| ATOM | 721 | N | LEU G | 158 | 41.279 | −5.588 | −4.192 | 1.00 | 39.52 | | N |
| ATOM | 722 | CA | LEU G | 158 | 41.557 | −6.817 | −3.448 | 1.00 | 50.27 | | C |
| ATOM | 723 | CB | LEU G | 158 | 41.899 | −7.952 | −4.419 | 1.00 | 20.00 | | C |
| ATOM | 724 | CG | LEU G | 158 | 40.817 | −8.321 | −5.435 | 1.00 | 20.00 | | C |
| ATOM | 725 | CD1 | LEU G | 158 | 41.287 | −9.458 | −6.330 | 1.00 | 20.00 | | C |
| ATOM | 726 | CD2 | LEU G | 158 | 39.521 | −8.689 | −4.729 | 1.00 | 20.00 | | C |
| ATOM | 727 | C | LEU G | 158 | 42.627 | −6.743 | −2.351 | 1.00 | 54.47 | | C |
| ATOM | 728 | O | LEU G | 158 | 42.456 | −7.343 | −1.285 | 1.00 | 49.26 | | O |
| ATOM | 729 | N | HIS G | 159 | 43.716 | −6.021 | −2.585 | 1.00 | 59.45 | | N |
| ATOM | 730 | CA | HIS G | 159 | 44.772 | −5.947 | −1.579 | 1.00 | 49.11 | | C |
| ATOM | 731 | CB | HIS G | 159 | 45.963 | −5.146 | −2.108 | 1.00 | 20.00 | | C |
| ATOM | 732 | CG | HIS G | 159 | 46.583 | −5.728 | −3.340 | 1.00 | 20.00 | | C |
| ATOM | 733 | ND1 | HIS G | 159 | 47.606 | −6.651 | −3.293 | 1.00 | 20.00 | | N |
| ATOM | 734 | CE1 | HIS G | 159 | 47.951 | −6.984 | −4.524 | 1.00 | 20.00 | | C |
| ATOM | 735 | NE2 | HIS G | 159 | 47.189 | −6.311 | −5.368 | 1.00 | 20.00 | | N |
| ATOM | 736 | CD2 | HIS G | 159 | 46.325 | −5.518 | −4.652 | 1.00 | 20.00 | | C |
| ATOM | 737 | C | HIS G | 159 | 44.256 | −5.325 | −0.280 | 1.00 | 44.09 | | C |
| ATOM | 738 | O | HIS G | 159 | 44.572 | −5.797 | 0.819 | 1.00 | 42.13 | | O |
| ATOM | 739 | N | LEU G | 160 | 43.449 | −4.277 | −0.410 | 1.00 | 37.55 | | N |
| ATOM | 740 | CA | LEU G | 160 | 42.876 | −3.609 | 0.751 | 1.00 | 43.52 | | C |
| ATOM | 741 | CB | LEU G | 160 | 42.095 | −2.365 | 0.323 | 1.00 | 20.00 | | C |
| ATOM | 742 | CG | LEU G | 160 | 42.891 | −1.283 | −0.409 | 1.00 | 20.00 | | C |
| ATOM | 743 | CD1 | LEU G | 160 | 41.996 | −0.107 | −0.769 | 1.00 | 20.00 | | C |
| ATOM | 744 | CD2 | LEU G | 160 | 44.073 | −0.825 | 0.432 | 1.00 | 20.00 | | C |
| ATOM | 745 | C | LEU G | 160 | 41.966 | −4.559 | 1.523 | 1.00 | 47.22 | | C |
| ATOM | 746 | O | LEU G | 160 | 41.969 | −4.578 | 2.756 | 1.00 | 43.25 | | O |
| ATOM | 747 | N | GLU G | 161 | 41.190 | −5.350 | 0.788 | 1.00 | 40.88 | | N |
| ATOM | 748 | CA | GLU G | 161 | 40.292 | −6.318 | 1.402 | 1.00 | 40.61 | | C |
| ATOM | 749 | CB | GLU G | 161 | 39.434 | −7.005 | 0.338 | 1.00 | 20.00 | | C |
| ATOM | 750 | CG | GLU G | 161 | 38.558 | −6.055 | −0.462 | 1.00 | 20.00 | | C |
| ATOM | 751 | CD | GLU G | 161 | 37.719 | −6.773 | −1.501 | 1.00 | 20.00 | | C |
| ATOM | 752 | OE1 | GLU G | 161 | 36.959 | −6.094 | −2.224 | 1.00 | 20.00 | | O |
| ATOM | 753 | OE2 | GLU G | 161 | 37.819 | −8.014 | −1.596 | 1.00 | 20.00 | | O |
| ATOM | 754 | C | GLU G | 161 | 41.097 | −7.352 | 2.179 | 1.00 | 39.76 | | C |
| ATOM | 755 | O | GLU G | 161 | 40.720 | −7.747 | 3.284 | 1.00 | 44.02 | | O |
| ATOM | 756 | N | GLY G | 162 | 42.213 | −7.781 | 1.599 | 1.00 | 34.18 | | N |
| ATOM | 757 | CA | GLY G | 162 | 43.079 | −8.742 | 2.256 | 1.00 | 35.01 | | C |
| ATOM | 758 | C | GLY G | 162 | 43.637 | −8.163 | 3.542 | 1.00 | 34.86 | | C |
| ATOM | 759 | O | GLY G | 162 | 43.706 | −8.844 | 4.570 | 1.00 | 47.81 | | O |
| ATOM | 760 | N | GLU G | 163 | 44.019 | −6.890 | 3.489 | 1.00 | 40.78 | | N |
| ATOM | 761 | CA | GLU G | 163 | 44.546 | −6.213 | 4.669 | 1.00 | 37.20 | | C |
| ATOM | 762 | CB | GLU G | 163 | 45.016 | −4.801 | 4.315 | 1.00 | 20.00 | | C |
| ATOM | 763 | CG | GLU G | 163 | 46.112 | −4.758 | 3.264 | 1.00 | 20.00 | | C |
| ATOM | 764 | CD | GLU G | 163 | 46.555 | −3.345 | 2.942 | 1.00 | 20.00 | | C |
| ATOM | 765 | OE1 | GLU G | 163 | 47.454 | −3.181 | 2.090 | 1.00 | 20.00 | | O |
| ATOM | 766 | OE2 | GLU G | 163 | 46.003 | −2.399 | 3.540 | 1.00 | 20.00 | | O |
| ATOM | 767 | C | GLU G | 163 | 43.480 | −6.158 | 5.758 | 1.00 | 46.12 | | C |
| ATOM | 768 | O | GLU G | 163 | 43.770 | −6.363 | 6.939 | 1.00 | 58.66 | | O |
| ATOM | 769 | N | VAL G | 164 | 42.243 | −5.885 | 5.354 | 1.00 | 42.23 | | N |
| ATOM | 770 | CA | VAL G | 164 | 41.129 | −5.833 | 6.292 | 1.00 | 35.45 | | C |
| ATOM | 771 | CB | VAL G | 164 | 39.827 | −5.387 | 5.602 | 1.00 | 20.00 | | C |
| ATOM | 772 | CG1 | VAL G | 164 | 38.684 | −5.343 | 6.604 | 1.00 | 20.00 | | C |
| ATOM | 773 | CG2 | VAL G | 164 | 40.015 | −4.032 | 4.937 | 1.00 | 20.00 | | C |
| ATOM | 774 | C | VAL G | 164 | 40.908 | −7.195 | 6.947 | 1.00 | 46.32 | | C |
| ATOM | 775 | O | VAL G | 164 | 40.656 | −7.284 | 8.153 | 1.00 | 45.08 | | O |
| ATOM | 776 | N | ASN G | 165 | 41.011 | −8.257 | 6.151 | 1.00 | 40.82 | | N |
| ATOM | 777 | CA | ASN G | 165 | 40.854 | −9.603 | 6.687 | 1.00 | 46.48 | | C |
| ATOM | 778 | CB | ASN G | 165 | 40.897 | −10.652 | 5.573 | 1.00 | 20.00 | | C |
| ATOM | 779 | CG | ASN G | 165 | 39.710 | −10.560 | 4.634 | 1.00 | 20.00 | | C |
| ATOM | 780 | OD1 | ASN G | 165 | 38.643 | −11.108 | 4.909 | 1.00 | 20.00 | | O |
| ATOM | 781 | ND2 | ASN G | 165 | 39.896 | −9.881 | 3.509 | 1.00 | 20.00 | | N |
| ATOM | 782 | C | ASN G | 165 | 41.947 | −9.884 | 7.708 | 1.00 | 50.90 | | C |
| ATOM | 783 | O | ASN G | 165 | 41.694 | −10.455 | 8.769 | 1.00 | 47.11 | | O |
| ATOM | 784 | N | LYS G | 166 | 43.161 | −9.451 | 7.385 | 1.00 | 48.36 | | N |
| ATOM | 785 | CA | LYS G | 166 | 44.305 | −9.640 | 8.269 | 1.00 | 38.71 | | C |
| ATOM | 786 | CB | LYS G | 166 | 45.591 | −9.154 | 7.595 | 1.00 | 20.00 | | C |
| ATOM | 787 | CG | LYS G | 166 | 45.907 | −9.850 | 6.277 | 1.00 | 20.00 | | C |
| ATOM | 788 | CD | LYS G | 166 | 47.180 | −9.298 | 5.649 | 1.00 | 20.00 | | C |
| ATOM | 789 | CE | LYS G | 166 | 47.495 | −9.990 | 4.331 | 1.00 | 20.00 | | C |
| ATOM | 790 | NZ | LYS G | 166 | 48.735 | −9.455 | 3.701 | 1.00 | 20.00 | | N |
| ATOM | 791 | C | LYS G | 166 | 44.090 | −8.907 | 9.591 | 1.00 | 45.07 | | C |
| ATOM | 792 | O | LYS G | 166 | 44.459 | −9.402 | 10.657 | 1.00 | 56.09 | | O |
| ATOM | 793 | N | ILE G | 167 | 43.491 | −7.725 | 9.508 | 1.00 | 42.02 | | N |
| ATOM | 794 | CA | ILE G | 167 | 43.256 | −6.876 | 10.673 | 1.00 | 43.86 | | C |
| ATOM | 795 | CB | ILE G | 167 | 43.338 | −5.380 | 10.304 | 1.00 | 20.00 | | C |
| ATOM | 796 | CG1 | ILE G | 167 | 44.721 | −5.042 | 9.743 | 1.00 | 20.00 | | C |
| ATOM | 797 | CD1 | ILE G | 167 | 44.884 | −3.589 | 9.349 | 1.00 | 20.00 | | C |
| ATOM | 798 | CG2 | ILE G | 167 | 43.025 | −4.514 | 11.516 | 1.00 | 20.00 | | C |
| ATOM | 799 | C | ILE G | 167 | 41.921 | −7.143 | 11.377 | 1.00 | 49.02 | | C |
| ATOM | 800 | O | ILE G | 167 | 41.570 | −6.443 | 12.327 | 1.00 | 37.71 | | O |
| ATOM | 801 | N | LYS G | 168 | 41.175 | −8.142 | 10.911 | 1.00 | 33.87 | | N |
| ATOM | 802 | CA | LYS G | 168 | 39.851 | −8.426 | 11.471 | 1.00 | 35.11 | | C |
| ATOM | 803 | CB | LYS G | 168 | 39.163 | −9.533 | 10.667 | 1.00 | 20.00 | | C |
| ATOM | 804 | CG | LYS G | 168 | 38.874 | −9.175 | 9.219 | 1.00 | 20.00 | | C |
| ATOM | 805 | CD | LYS G | 168 | 38.101 | −10.286 | 8.524 | 1.00 | 20.00 | | C |
| ATOM | 806 | CE | LYS G | 168 | 37.755 | −9.909 | 7.092 | 1.00 | 20.00 | | C |
| ATOM | 807 | NZ | LYS G | 168 | 36.921 | −10.950 | 6.427 | 1.00 | 20.00 | | N |
| ATOM | 808 | C | LYS G | 168 | 39.820 | −8.790 | 12.966 | 1.00 | 39.27 | | C |
| ATOM | 809 | O | LYS G | 168 | 38.932 | −8.339 | 13.690 | 1.00 | 32.39 | | O |
| ATOM | 810 | N | SER G | 169 | 40.770 | −9.599 | 13.428 | 1.00 | 41.30 | | N |
| ATOM | 811 | CA | SER G | 169 | 40.805 | −10.008 | 14.827 | 1.00 | 43.23 | | C |
| ATOM | 812 | CB | SER G | 169 | 42.030 | −10.884 | 15.100 | 1.00 | 20.00 | | C |
| ATOM | 813 | OG | SER G | 169 | 43.231 | −10.175 | 14.847 | 1.00 | 20.00 | | O |
| ATOM | 814 | C | SER G | 169 | 40.796 | −8.806 | 15.767 | 1.00 | 41.44 | | C |
| ATOM | 815 | O | SER G | 169 | 40.016 | −8.759 | 16.718 | 1.00 | 44.82 | | O |
| ATOM | 816 | N | ALA G | 170 | 41.695 | −7.857 | 15.522 | 1.00 | 39.78 | | N |
| ATOM | 817 | CA | ALA G | 170 | 41.753 | −6.638 | 16.319 | 1.00 | 41.01 | | C |
| ATOM | 818 | CB | ALA G | 170 | 42.928 | −5.775 | 15.887 | 1.00 | 20.00 | | C |
| ATOM | 819 | C | ALA G | 170 | 40.448 | −5.857 | 16.211 | 1.00 | 46.04 | | C |
| ATOM | 820 | O | ALA G | 170 | 39.912 | −5.381 | 17.211 | 1.00 | 46.67 | | O |
| ATOM | 821 | N | LEU G | 171 | 39.921 | −5.766 | 14.995 | 1.00 | 34.26 | | N |
| ATOM | 822 | CA | LEU G | 171 | 38.642 | −5.108 | 14.762 | 1.00 | 37.69 | | C |
| ATOM | 823 | CB | LEU G | 171 | 38.269 | −5.179 | 13.280 | 1.00 | 20.00 | | C |
| ATOM | 824 | CG | LEU G | 171 | 39.239 | −4.485 | 12.323 | 1.00 | 20.00 | | C |
| ATOM | 825 | CD1 | LEU G | 171 | 38.764 | −4.625 | 10.881 | 1.00 | 20.00 | | C |
| ATOM | 826 | CD2 | LEU G | 171 | 39.417 | −3.022 | 12.711 | 1.00 | 20.00 | | C |
| ATOM | 827 | C | LEU G | 171 | 37.534 | −5.723 | 15.616 | 1.00 | 44.67 | | C |
| ATOM | 828 | O | LEU G | 171 | 36.683 | −5.010 | 16.151 | 1.00 | 34.27 | | O |
| ATOM | 829 | N | LEU G | 172 | 37.577 | −7.043 | 15.776 | 1.00 | 34.80 | | N |
| ATOM | 830 | CA | LEU G | 172 | 36.536 | −7.765 | 16.498 | 1.00 | 28.87 | | C |
| ATOM | 831 | CB | LEU G | 172 | 36.606 | −9.258 | 16.179 | 1.00 | 20.00 | | C |
| ATOM | 832 | CG | LEU G | 172 | 36.254 | −9.646 | 14.744 | 1.00 | 20.00 | | C |
| ATOM | 833 | CD1 | LEU G | 172 | 36.412 | −11.143 | 14.539 | 1.00 | 20.00 | | C |
| ATOM | 834 | CD2 | LEU G | 172 | 34.842 | −9.201 | 14.414 | 1.00 | 20.00 | | C |
| ATOM | 835 | C | LEU G | 172 | 36.655 | −7.547 | 18.001 | 1.00 | 29.90 | | C |
| ATOM | 836 | O | LEU G | 172 | 35.650 | −7.435 | 18.704 | 1.00 | 41.09 | | O |
| ATOM | 837 | N | SER G | 173 | 37.889 | −7.498 | 18.490 | 1.00 | 35.08 | | N |
| ATOM | 838 | CA | SER G | 173 | 38.139 | −7.406 | 19.923 | 1.00 | 35.53 | | C |
| ATOM | 839 | CB | SER G | 173 | 39.578 | −7.823 | 20.245 | 1.00 | 20.00 | | C |
| ATOM | 840 | OG | SER G | 173 | 40.515 | −7.012 | 19.556 | 1.00 | 20.00 | | O |
| ATOM | 841 | C | SER G | 173 | 37.860 | −5.999 | 20.450 | 1.00 | 35.07 | | C |
| ATOM | 842 | O | SER G | 173 | 37.581 | −5.815 | 21.636 | 1.00 | 51.28 | | O |
| ATOM | 843 | N | THR G | 174 | 37.893 | −5.021 | 19.550 | 1.00 | 46.35 | | N |
| ATOM | 844 | CA | THR G | 174 | 37.721 | −3.621 | 19.922 | 1.00 | 43.83 | | C |
| ATOM | 845 | CB | THR G | 174 | 38.341 | −2.681 | 18.860 | 1.00 | 20.00 | | C |
| ATOM | 846 | OG1 | THR G | 174 | 37.702 | −2.897 | 17.595 | 1.00 | 20.00 | | O |
| ATOM | 847 | CG2 | THR G | 174 | 39.839 | −2.940 | 18.719 | 1.00 | 20.00 | | C |
| ATOM | 848 | C | THR G | 174 | 36.236 | −3.300 | 20.083 | 1.00 | 52.54 | | C |
| ATOM | 849 | O | THR G | 174 | 35.448 | −3.528 | 19.165 | 1.00 | 64.10 | | O |
| ATOM | 850 | N | ASN G | 175 | 35.839 | −2.919 | 21.295 | 1.00 | 55.57 | | N |
| ATOM | 851 | CA | ASN G | 175 | 34.501 | −2.371 | 21.528 | 1.00 | 40.58 | | C |
| ATOM | 852 | CB | ASN G | 175 | 34.109 | −2.499 | 23.005 | 1.00 | 20.00 | | C |
| ATOM | 853 | CG | ASN G | 175 | 33.819 | −3.933 | 23.416 | 1.00 | 20.00 | | C |
| ATOM | 854 | OD1 | ASN G | 175 | 33.530 | −4.786 | 22.578 | 1.00 | 20.00 | | O |
| ATOM | 855 | ND2 | ASN G | 175 | 33.845 | −4.190 | 24.720 | 1.00 | 20.00 | | N |
| ATOM | 856 | C | ASN G | 175 | 34.404 | −0.909 | 21.089 | 1.00 | 43.65 | | C |
| ATOM | 857 | O | ASN G | 175 | 35.035 | −0.032 | 21.682 | 1.00 | 53.11 | | O |
| ATOM | 858 | N | LYS G | 176 | 33.694 | −0.671 | 19.990 | 1.00 | 44.89 | | N |
| ATOM | 859 | CA | LYS G | 176 | 33.519 | 0.680 | 19.463 | 1.00 | 46.42 | | C |
| ATOM | 860 | CB | LYS G | 176 | 34.847 | 1.244 | 18.945 | 1.00 | 20.00 | | C |
| ATOM | 861 | CG | LYS G | 176 | 35.789 | 1.756 | 20.031 | 1.00 | 20.00 | | C |
| ATOM | 862 | CD | LYS G | 176 | 37.220 | 1.872 | 19.511 | 1.00 | 20.00 | | C |
| ATOM | 863 | CE | LYS G | 176 | 38.243 | 1.433 | 20.560 | 1.00 | 20.00 | | C |
| ATOM | 864 | NZ | LYS G | 176 | 39.542 | 1.012 | 19.953 | 1.00 | 20.00 | | N |
| ATOM | 865 | C | LYS G | 176 | 32.478 | 0.701 | 18.352 | 1.00 | 45.00 | | C |
| ATOM | 866 | O | LYS G | 176 | 32.405 | −0.217 | 17.534 | 1.00 | 60.64 | | O |
| ATOM | 867 | N | ALA G | 177 | 31.674 | 1.756 | 18.330 | 1.00 | 46.01 | | N |
| ATOM | 868 | CA | ALA G | 177 | 30.717 | 1.962 | 17.257 | 1.00 | 43.65 | | C |
| ATOM | 869 | CB | ALA G | 177 | 29.708 | 3.027 | 17.652 | 1.00 | 20.00 | | C |
| ATOM | 870 | C | ALA G | 177 | 31.437 | 2.359 | 15.977 | 1.00 | 41.42 | | C |
| ATOM | 871 | O | ALA G | 177 | 31.168 | 1.813 | 14.907 | 1.00 | 45.37 | | O |
| ATOM | 872 | N | VAL G | 178 | 32.360 | 3.308 | 16.097 | 1.00 | 35.70 | | N |
| ATOM | 873 | CA | VAL G | 178 | 33.228 | 3.683 | 14.987 | 1.00 | 43.93 | | C |
| ATOM | 874 | CB | VAL G | 178 | 32.876 | 5.083 | 14.432 | 1.00 | 20.00 | | C |
| ATOM | 875 | CG1 | VAL G | 178 | 33.896 | 5.516 | 13.379 | 1.00 | 20.00 | | C |
| ATOM | 876 | CG2 | VAL G | 178 | 31.463 | 5.090 | 13.860 | 1.00 | 20.00 | | C |
| ATOM | 877 | C | VAL G | 178 | 34.685 | 3.676 | 15.427 | 1.00 | 34.16 | | C |
| ATOM | 878 | O | VAL G | 178 | 35.005 | 4.063 | 16.550 | 1.00 | 44.45 | | O |
| ATOM | 879 | N | VAL G | 179 | 35.560 | 3.200 | 14.550 | 1.00 | 44.87 | | N |
| ATOM | 880 | CA | VAL G | 179 | 36.972 | 3.080 | 14.884 | 1.00 | 38.47 | | C |
| ATOM | 881 | CB | VAL G | 179 | 37.255 | 1.806 | 15.696 | 1.00 | 20.00 | | C |
| ATOM | 882 | CG1 | VAL G | 179 | 38.742 | 1.679 | 15.986 | 1.00 | 20.00 | | C |
| ATOM | 883 | CG2 | VAL G | 179 | 36.457 | 1.817 | 16.984 | 1.00 | 20.00 | | C |
| ATOM | 884 | C | VAL G | 179 | 37.855 | 3.091 | 13.643 | 1.00 | 44.79 | | C |
| ATOM | 885 | O | VAL G | 179 | 37.560 | 2.423 | 12.650 | 1.00 | 51.88 | | O |
| ATOM | 886 | N | SER G | 180 | 38.955 | 3.835 | 13.719 | 1.00 | 45.23 | | N |
| ATOM | 887 | CA | SER G | 180 | 39.937 | 3.871 | 12.643 | 1.00 | 48.99 | | C |
| ATOM | 888 | CB | SER G | 180 | 40.875 | 5.069 | 12.812 | 1.00 | 20.00 | | C |
| ATOM | 889 | OG | SER G | 180 | 41.649 | 4.948 | 13.993 | 1.00 | 20.00 | | O |
| ATOM | 890 | C | SER G | 180 | 40.740 | 2.577 | 12.610 | 1.00 | 48.15 | | C |
| ATOM | 891 | O | SER G | 180 | 41.386 | 2.212 | 13.594 | 1.00 | 60.42 | | O |
| ATOM | 892 | N | LEU G | 181 | 40.594 | 1.827 | 11.521 | 1.00 | 42.42 | | N |
| ATOM | 893 | CA | LEU G | 181 | 41.389 | 0.622 | 11.302 | 1.00 | 43.22 | | C |
| ATOM | 894 | CB | LEU G | 181 | 40.818 | −0.190 | 10.139 | 1.00 | 20.00 | | C |
| ATOM | 895 | CG | LEU G | 181 | 39.415 | −0.753 | 10.350 | 1.00 | 20.00 | | C |
| ATOM | 896 | CD1 | LEU G | 181 | 38.958 | −1.514 | 9.118 | 1.00 | 20.00 | | C |
| ATOM | 897 | CD2 | LEU G | 181 | 39.394 | −1.643 | 11.577 | 1.00 | 20.00 | | C |
| ATOM | 898 | C | LEU G | 181 | 42.838 | 0.984 | 11.011 | 1.00 | 29.14 | | C |
| ATOM | 899 | O | LEU G | 181 | 43.144 | 1.559 | 9.967 | 1.00 | 43.42 | | O |
| ATOM | 900 | N | SER G | 182 | 43.721 | 0.695 | 11.960 | 1.00 | 41.83 | | N |
| ATOM | 901 | CA | SER G | 182 | 45.089 | 1.186 | 11.883 | 1.00 | 53.52 | | C |
| ATOM | 902 | CB | SER G | 182 | 45.421 | 2.069 | 13.086 | 1.00 | 20.00 | | C |
| ATOM | 903 | OG | SER G | 182 | 45.500 | 1.303 | 14.275 | 1.00 | 20.00 | | O |
| ATOM | 904 | C | SER G | 182 | 46.110 | 0.063 | 11.741 | 1.00 | 44.76 | | C |
| ATOM | 905 | O | SER G | 182 | 45.995 | −0.984 | 12.379 | 1.00 | 37.73 | | O |
| ATOM | 906 | N | ASN G | 183 | 47.028 | 0.245 | 10.799 | 1.00 | 47.84 | | N |
| ATOM | 907 | CA | ASN G | 183 | 48.262 | −0.525 | 10.744 | 1.00 | 59.45 | | C |
| ATOM | 908 | CB | ASN G | 183 | 48.350 | −1.276 | 9.412 | 1.00 | 20.00 | | C |
| ATOM | 909 | CG | ASN G | 183 | 47.312 | −2.375 | 9.290 | 1.00 | 20.00 | | C |
| ATOM | 910 | OD1 | ASN G | 183 | 46.897 | −2.964 | 10.287 | 1.00 | 20.00 | | O |
| ATOM | 911 | ND2 | ASN G | 183 | 46.954 | −2.715 | 8.057 | 1.00 | 20.00 | | N |
| ATOM | 912 | C | ASN G | 183 | 49.445 | 0.428 | 10.872 | 1.00 | 75.41 | | C |
| ATOM | 913 | O | ASN G | 183 | 49.386 | 1.559 | 10.391 | 1.00 | 102.31 | | O |
| ATOM | 914 | N | GLY G | 184 | 50.463 | 0.021 | 11.624 | 1.00 | 79.30 | | N |
| ATOM | 915 | CA | GLY G | 184 | 51.584 | 0.906 | 11.931 | 1.00 | 88.92 | | C |
| ATOM | 916 | C | GLY G | 184 | 51.464 | 2.278 | 11.287 | 1.00 | 101.85 | | C |
| ATOM | 917 | O | GLY G | 184 | 51.911 | 2.485 | 10.158 | 1.00 | 113.48 | O | |
| ATOM | 918 | N | VAL G | 185 | 50.833 | 3.209 | 11.999 | 1.00 | 86.84 | | N |
| ATOM | 919 | CA | VAL G | 185 | 50.888 | 4.635 | 11.665 | 1.00 | 81.80 | | C |
| ATOM | 920 | CB | VAL G | 185 | 52.336 | 5.149 | 11.526 | 1.00 | 20.00 | | C |
| ATOM | 921 | CG1 | VAL G | 185 | 52.340 | 6.607 | 11.085 | 1.00 | 20.00 | | C |
| ATOM | 922 | CG2 | VAL G | 185 | 53.086 | 4.984 | 12.838 | 1.00 | 20.00 | | C |
| ATOM | 923 | C | VAL G | 185 | 50.097 | 5.027 | 10.417 | 1.00 | 81.69 | | C |
| ATOM | 924 | O | VAL G | 185 | 50.205 | 6.157 | 9.942 | 1.00 | 99.84 | | O |
| ATOM | 925 | N | SER G | 186 | 49.286 | 4.109 | 9.902 | 1.00 | 70.63 | | N |
| ATOM | 926 | CA | SER G | 186 | 48.501 | 4.392 | 8.705 | 1.00 | 72.29 | | C |
| ATOM | 927 | CB | SER G | 186 | 49.173 | 3.803 | 7.463 | 1.00 | 20.00 | | C |
| ATOM | 928 | OG | SER G | 186 | 49.228 | 2.389 | 7.534 | 1.00 | 20.00 | | O |
| ATOM | 929 | C | SER G | 186 | 47.066 | 3.893 | 8.820 | 1.00 | 62.99 | | C |
| ATOM | 930 | O | SER G | 186 | 46.820 | 2.769 | 9.258 | 1.00 | 58.35 | | O |
| ATOM | 931 | N | VAL G | 187 | 46.122 | 4.736 | 8.415 | 1.00 | 66.49 | | N |
| ATOM | 932 | CA | VAL G | 187 | 44.709 | 4.389 | 8.471 | 1.00 | 57.36 | | C |
| ATOM | 933 | CB | VAL G | 187 | 43.834 | 5.635 | 8.704 | 1.00 | 20.00 | | C |
| ATOM | 934 | CG1 | VAL G | 187 | 42.371 | 5.248 | 8.769 | 1.00 | 20.00 | | C |
| ATOM | 935 | CG2 | VAL G | 187 | 44.254 | 6.346 | 9.982 | 1.00 | 20.00 | | C |
| ATOM | 936 | C | VAL G | 187 | 44.266 | 3.684 | 7.191 | 1.00 | 53.67 | | C |
| ATOM | 937 | O | VAL G | 187 | 44.408 | 4.222 | 6.093 | 1.00 | 50.10 | | O |
| ATOM | 938 | N | LEU G | 188 | 43.768 | 2.460 | 7.338 | 1.00 | 43.20 | | N |
| ATOM | 939 | CA | LEU G | 188 | 43.289 | 1.678 | 6.201 | 1.00 | 41.76 | | C |
| ATOM | 940 | CB | LEU G | 188 | 43.404 | 0.183 | 6.495 | 1.00 | 20.00 | | C |
| ATOM | 941 | CG | LEU G | 188 | 44.821 | −0.375 | 6.633 | 1.00 | 20.00 | | C |
| ATOM | 942 | CD1 | LEU G | 188 | 44.777 | −1.845 | 7.014 | 1.00 | 20.00 | | C |
| ATOM | 943 | CD2 | LEU G | 188 | 45.605 | −0.174 | 5.345 | 1.00 | 20.00 | | C |
| ATOM | 944 | C | LEU G | 188 | 41.843 | 2.021 | 5.862 | 1.00 | 44.75 | | C |
| ATOM | 945 | O | LEU G | 188 | 41.384 | 1.777 | 4.746 | 1.00 | 45.28 | | O |
| ATOM | 946 | N | THR G | 189 | 41.111 | 2.500 | 6.863 | 1.00 | 39.28 | | N |
| ATOM | 947 | CA | THR G | 189 | 39.710 | 2.863 | 6.694 | 1.00 | 36.51 | | C |
| ATOM | 948 | CB | THR G | 189 | 38.985 | 1.880 | 5.760 | 1.00 | 20.00 | | C |
| ATOM | 949 | OG1 | THR G | 189 | 38.871 | 0.604 | 6.400 | 1.00 | 20.00 | | O |
| ATOM | 950 | CG2 | THR G | 189 | 39.759 | 1.715 | 4.458 | 1.00 | 20.00 | | C |
| ATOM | 951 | C | THR G | 189 | 39.006 | 2.891 | 8.046 | 1.00 | 45.83 | | C |
| ATOM | 952 | O | THR G | 189 | 39.637 | 3.112 | 9.079 | 1.00 | 42.57 | | O |
| ATOM | 953 | N | SER G | 190 | 37.692 | 2.695 | 8.033 | 1.00 | 40.44 | | N |
| ATOM | 954 | CA | SER G | 190 | 36.902 | 2.779 | 9.255 | 1.00 | 41.82 | | C |
| ATOM | 955 | CB | SER G | 190 | 35.919 | 3.953 | 9.188 | 1.00 | 20.00 | | C |
| ATOM | 956 | OG | SER G | 190 | 34.930 | 3.737 | 8.196 | 1.00 | 20.00 | | O |
| ATOM | 957 | C | SER G | 190 | 36.153 | 1.479 | 9.527 | 1.00 | 33.81 | | C |
| ATOM | 958 | O | SER G | 190 | 35.609 | 0.860 | 8.611 | 1.00 | 49.61 | | O |
| ATOM | 959 | N | LYS G | 191 | 36.134 | 1.069 | 10.791 | 1.00 | 37.37 | | N |
| ATOM | 960 | CA | LYS G | 191 | 35.365 | −0.097 | 11.208 | 1.00 | 41.23 | | C |
| ATOM | 961 | CB | LYS G | 191 | 36.239 | −1.047 | 12.027 | 1.00 | 20.00 | | C |
| ATOM | 962 | CG | LYS G | 191 | 37.323 | −1.731 | 11.216 | 1.00 | 20.00 | | C |
| ATOM | 963 | CD | LYS G | 191 | 38.193 | −2.623 | 12.085 | 1.00 | 20.00 | | C |
| ATOM | 964 | CE | LYS G | 191 | 39.313 | −3.257 | 11.267 | 1.00 | 20.00 | | C |
| ATOM | 965 | NZ | LYS G | 191 | 40.316 | −3.959 | 12.119 | 1.00 | 20.00 | | N |
| ATOM | 966 | C | LYS G | 191 | 34.143 | 0.315 | 12.018 | 1.00 | 35.03 | | C |
| ATOM | 967 | O | LYS G | 191 | 34.267 | 0.958 | 13.059 | 1.00 | 40.06 | | O |
| ATOM | 968 | N | VAL G | 192 | 32.966 | −0.093 | 11.555 | 1.00 | 33.70 | | N |
| ATOM | 969 | CA | VAL G | 192 | 31.712 | 0.327 | 12.171 | 1.00 | 27.61 | | C |
| ATOM | 970 | CB | VAL G | 192 | 30.816 | 1.081 | 11.173 | 1.00 | 20.00 | | C |
| ATOM | 971 | CG1 | VAL G | 192 | 29.505 | 1.481 | 11.832 | 1.00 | 20.00 | | C |
| ATOM | 972 | CG2 | VAL G | 192 | 31.540 | 2.293 | 10.617 | 1.00 | 20.00 | | C |
| ATOM | 973 | C | VAL G | 192 | 30.929 | −0.857 | 12.723 | 1.00 | 31.74 | | C |
| ATOM | 974 | O | VAL G | 192 | 30.965 | −1.954 | 12.167 | 1.00 | 25.58 | | O |
| ATOM | 975 | N | LEU G | 193 | 30.177 | −0.610 | 13.790 | 1.00 | 28.88 | | N |
| ATOM | 976 | CA | LEU G | 193 | 29.272 | −1.609 | 14.342 | 1.00 | 34.36 | | C |
| ATOM | 977 | CB | LEU G | 193 | 29.940 | −2.349 | 15.504 | 1.00 | 20.00 | | C |
| ATOM | 978 | CG | LEU G | 193 | 31.127 | −3.249 | 15.156 | 1.00 | 20.00 | | C |
| ATOM | 979 | CD1 | LEU G | 193 | 31.818 | −3.743 | 16.418 | 1.00 | 20.00 | | C |
| ATOM | 980 | CD2 | LEU G | 193 | 30.677 | −4.419 | 14.295 | 1.00 | 20.00 | | C |
| ATOM | 981 | C | LEU G | 193 | 27.982 | −0.950 | 14.818 | 1.00 | 33.03 | | C |
| ATOM | 982 | O | LEU G | 193 | 27.963 | −0.290 | 15.856 | 1.00 | 44.93 | | O |
| ATOM | 983 | N | ASP G | 194 | 26.896 | −1.170 | 14.081 | 1.00 | 35.09 | | N |
| ATOM | 984 | CA | ASP G | 194 | 25.724 | −0.298 | 14.150 | 1.00 | 41.39 | | C |
| ATOM | 985 | CB | ASP G | 194 | 24.766 | −0.589 | 12.995 | 1.00 | 20.00 | | C |
| ATOM | 986 | CG | ASP G | 194 | 25.292 | −0.090 | 11.667 | 1.00 | 20.00 | | C |
| ATOM | 987 | OD1 | ASP G | 194 | 26.173 | 0.795 | 11.668 | 1.00 | 20.00 | | O |
| ATOM | 988 | OD2 | ASP G | 194 | 24.863 | −0.620 | 10.621 | 1.00 | 20.00 | | O |
| ATOM | 989 | C | ASP G | 194 | 24.976 | −0.377 | 15.479 | 1.00 | 38.04 | | C |
| ATOM | 990 | O | ASP G | 194 | 24.097 | 0.442 | 15.752 | 1.00 | 48.03 | | O |
| ATOM | 991 | N | LEU G | 195 | 25.259 | −1.410 | 16.263 | 1.00 | 43.64 | | N |
| ATOM | 992 | CA | LEU G | 195 | 24.463 | −1.682 | 17.449 | 1.00 | 35.97 | | C |
| ATOM | 993 | CB | LEU G | 195 | 23.832 | −3.071 | 17.366 | 1.00 | 20.00 | | C |
| ATOM | 994 | CG | LEU G | 195 | 22.988 | −3.340 | 16.117 | 1.00 | 20.00 | | C |
| ATOM | 995 | CD1 | LEU G | 195 | 22.479 | −4.773 | 16.112 | 1.00 | 20.00 | | C |
| ATOM | 996 | CD2 | LEU G | 195 | 21.847 | −2.340 | 15.969 | 1.00 | 20.00 | | C |
| ATOM | 997 | C | LEU G | 195 | 25.254 | −1.525 | 18.744 | 1.00 | 43.63 | | C |
| ATOM | 998 | O | LEU G | 195 | 24.675 | −1.495 | 19.830 | 1.00 | 34.71 | | O |
| ATOM | 999 | N | ASN G | 196 | 26.558 | −1.299 | 18.617 | 1.00 | 44.30 | | N |
| ATOM | 1000 | CA | ASN G | 196 | 27.458 | −1.365 | 19.765 | 1.00 | 42.38 | | C |
| ATOM | 1001 | CB | ASNG | 196 | 28.900 | −1.082 | 19.342 | 1.00 | 20.00 | | C |
| ATOM | 1002 | CG | ASN G | 196 | 29.508 | −2.223 | 18.551 | 1.00 | 20.00 | | C |
| ATOM | 1003 | OD1 | ASN G | 196 | 29.101 | −3.377 | 18.688 | 1.00 | 20.00 | | O |
| ATOM | 1004 | ND2 | ASN G | 196 | 30.503 | −1.908 | 17.731 | 1.00 | 20.00 | | N |
| ATOM | 1005 | C | ASN G | 196 | 27.041 | −0.434 | 20.900 | 1.00 | 43.66 | | C |
| ATOM | 1006 | O | ASN G | 196 | 26.944 | −0.855 | 22.053 | 1.00 | 33.62 | | O |
| ATOM | 1007 | N | ASN G | 197 | 26.706 | 0.806 | 20.560 | 1.00 | 34.31 | | N |
| ATOM | 1008 | CA | ASN G | 197 | 26.346 | 1.793 | 21.570 | 1.00 | 38.83 | | C |
| ATOM | 1009 | CB | ASN G | 197 | 26.089 | 3.153 | 20.926 | 1.00 | 20.00 | | C |
| ATOM | 1010 | CG | ASN G | 197 | 27.362 | 3.819 | 20.456 | 1.00 | 20.00 | | C |
| ATOM | 1011 | OD1 | ASN G | 197 | 28.459 | 3.458 | 20.880 | 1.00 | 20.00 | | O |
| ATOM | 1012 | ND2 | ASN G | 197 | 27.228 | 4.768 | 19.541 | 1.00 | 20.00 | | N |
| ATOM | 1013 | C | ASN G | 197 | 25.139 | 1.365 | 22.392 | 1.00 | 39.90 | | C |
| ATOM | 1014 | O | ASN G | 197 | 25.185 | 1.357 | 23.621 | 1.00 | 39.04 | | O |
| ATOM | 1015 | N | TYR G | 198 | 24.065 | 0.992 | 21.705 | 1.00 | 35.09 | | N |
| ATOM | 1016 | CA | TYR G | 198 | 22.875 | 0.482 | 22.373 | 1.00 | 41.03 | | C |
| ATOM | 1017 | CB | TYR G | 198 | 21.804 | 0.093 | 21.352 | 1.00 | 20.00 | | C |
| ATOM | 1018 | CG | TYR G | 198 | 21.274 | 1.257 | 20.549 | 1.00 | 20.00 | | C |
| ATOM | 1019 | CD1 | TYR G | 198 | 20.238 | 2.041 | 21.031 | 1.00 | 20.00 | | C |
| ATOM | 1020 | CE1 | TYR G | 198 | 19.753 | 3.105 | 20.299 | 1.00 | 20.00 | | C |
| ATOM | 1021 | CZ | TYR G | 198 | 20.310 | 3.398 | 19.069 | 1.00 | 20.00 | | C |
| ATOM | 1022 | OH | TYR G | 198 | 19.829 | 4.450 | 18.325 | 1.00 | 20.00 | | O |
| ATOM | 1023 | CE2 | TYR G | 198 | 21.321 | 2.616 | 18.559 | 1.00 | 20.00 | | C |
| ATOM | 1024 | CD2 | TYR G | 198 | 21.799 | 1.557 | 19.299 | 1.00 | 20.00 | | C |
| ATOM | 1025 | C | TYR G | 198 | 23.219 | −0.712 | 23.252 | 1.00 | 43.65 | | C |
| ATOM | 1026 | O | TYR G | 198 | 22.800 | −0.783 | 24.406 | 1.00 | 52.37 | | O |
| ATOM | 1027 | N | ILE G | 199 | 24.079 | −1.587 | 22.744 | 1.00 | 35.86 | | N |
| ATOM | 1028 | CA | ILE G | 199 | 24.490 | −2.772 | 23.486 | 1.00 | 45.39 | | C |
| ATOM | 1029 | CB | ILE G | 199 | 25.380 | −3.704 | 22.627 | 1.00 | 20.00 | | C |
| ATOM | 1030 | CG1 | ILE G | 199 | 24.556 | −4.357 | 21.512 | 1.00 | 20.00 | | C |
| ATOM | 1031 | CD1 | ILE G | 199 | 25.365 | −5.260 | 20.598 | 1.00 | 20.00 | | C |
| ATOM | 1032 | CG2 | ILE G | 199 | 26.037 | −4.775 | 23.487 | 1.00 | 20.00 | | C |
| ATOM | 1033 | C | ILE G | 199 | 25.210 | −2.392 | 24.779 | 1.00 | 37.89 | | C |
| ATOM | 1034 | O | ILE G | 199 | 24.808 | −2.808 | 25.865 | 1.00 | 45.28 | | O |
| ATOM | 1035 | N | ASP G | 200 | 26.185 | −1.495 | 24.669 | 1.00 | 37.36 | | N |
| ATOM | 1036 | CA | ASP G | 200 | 26.988 | −1.098 | 25.821 | 1.00 | 46.66 | | C |
| ATOM | 1037 | CB | ASP G | 200 | 28.069 | −0.096 | 25.405 | 1.00 | 20.00 | | C |
| ATOM | 1038 | CG | ASP G | 200 | 29.216 | −0.751 | 24.660 | 1.00 | 20.00 | | C |
| ATOM | 1039 | OD1 | ASP G | 200 | 29.360 | −1.989 | 24.751 | 1.00 | 20.00 | | O |
| ATOM | 1040 | OD2 | ASP G | 200 | 29.996 | −0.025 | 24.009 | 1.00 | 20.00 | | O |
| ATOM | 1041 | C | ASP G | 200 | 26.109 | −0.506 | 26.916 | 1.00 | 51.05 | | C |
| ATOM | 1042 | O | ASP G | 200 | 26.370 | −0.693 | 28.105 | 1.00 | 57.39 | | O |
| ATOM | 1043 | N | LYS G | 201 | 25.004 | 0.108 | 26.507 | 1.00 | 57.68 | | N |
| ATOM | 1044 | CA | LYS G | 201 | 24.104 | 0.766 | 27.443 | 1.00 | 44.02 | | C |
| ATOM | 1045 | CB | LYS G | 201 | 23.138 | 1.687 | 26.698 | 1.00 | 20.00 | | C |
| ATOM | 1046 | CG | LYS G | 201 | 23.815 | 2.816 | 25.941 | 1.00 | 20.00 | | C |
| ATOM | 1047 | CD | LYS G | 201 | 22.797 | 3.680 | 25.223 | 1.00 | 20.00 | | C |
| ATOM | 1048 | CE | LYS G | 201 | 23.464 | 4.802 | 24.446 | 1.00 | 20.00 | | C |
| ATOM | 1049 | NZ | LYS G | 201 | 22.489 | 5.542 | 23.596 | 1.00 | 20.00 | | N |
| ATOM | 1050 | C | LYS G | 201 | 23.325 | −0.250 | 28.274 | 1.00 | 43.56 | | C |
| ATOM | 1051 | O | LYS G | 201 | 22.868 | 0.060 | 29.374 | 1.00 | 51.34 | | O |
| ATOM | 1052 | N | GLN G | 202 | 23.218 | −1.475 | 27.764 | 1.00 | 46.06 | | N |
| ATOM | 1053 | CA | GLN G | 202 | 22.308 | −2.472 | 28.333 | 1.00 | 44.95 | | C |
| ATOM | 1054 | CB | GLN G | 202 | 21.324 | −2.975 | 27.277 | 1.00 | 20.00 | | C |
| ATOM | 1055 | CG | GLN G | 202 | 20.207 | −1.997 | 26.966 | 1.00 | 20.00 | | C |
| ATOM | 1056 | CD | GLN G | 202 | 19.339 | −2.450 | 25.809 | 1.00 | 20.00 | | C |
| ATOM | 1057 | OE1 | GLN G | 202 | 19.694 | −3.372 | 25.074 | 1.00 | 20.00 | | O |
| ATOM | 1058 | NE2 | GLN G | 202 | 18.171 | −1.835 | 25.672 | 1.00 | 20.00 | | N |
| ATOM | 1059 | C | GLN G | 202 | 23.029 | −3.657 | 28.974 | 1.00 | 42.35 | | C |
| ATOM | 1060 | O | GLN G | 202 | 22.446 | −4.374 | 29.787 | 1.00 | 47.00 | | O |
| ATOM | 1061 | N | LEU G | 203 | 24.253 | −3.922 | 28.530 | 1.00 | 49.55 | | N |
| ATOM | 1062 | CA | LEU G | 203 | 25.003 | −5.081 | 29.008 | 1.00 | 50.77 | | C |
| ATOM | 1063 | CB | LEU G | 203 | 26.244 | −5.322 | 28.140 | 1.00 | 20.00 | | C |
| ATOM | 1064 | CG | LEU G | 203 | 26.031 | −5.802 | 26.698 | 1.00 | 20.00 | | C |
| ATOM | 1065 | CD1 | LEU G | 203 | 27.361 | −5.942 | 25.970 | 1.00 | 20.00 | | C |
| ATOM | 1066 | CD2 | LEU G | 203 | 25.263 | −7.115 | 26.661 | 1.00 | 20.00 | | C |
| ATOM | 1067 | C | LEU G | 203 | 25.397 | −4.938 | 30.479 | 1.00 | 52.51 | | C |
| ATOM | 1068 | O | LEU G | 203 | 25.830 | −3.873 | 30.918 | 1.00 | 46.02 | | O |
| ATOM | 1069 | N | LEU G | 204 | 25.226 | −6.017 | 31.238 | 1.00 | 51.45 | | N |
| ATOM | 1070 | CA | LEU G | 204 | 25.644 | −6.053 | 32.635 | 1.00 | 58.44 | | C |
| ATOM | 1071 | CB | LEU G | 204 | 24.863 | −7.127 | 33.399 | 1.00 | 20.00 | | C |
| ATOM | 1072 | CG | LEU G | 204 | 23.352 | −6.918 | 33.522 | 1.00 | 20.00 | | C |
| ATOM | 1073 | CD1 | LEU G | 204 | 22.691 | −8.136 | 34.147 | 1.00 | 20.00 | | C |
| ATOM | 1074 | CD2 | LEU G | 204 | 23.044 | −5.665 | 34.328 | 1.00 | 20.00 | | C |
| ATOM | 1075 | C | LEU G | 204 | 27.146 | −6.307 | 32.759 | 1.00 | 54.99 | | C |
| ATOM | 1076 | O | LEU G | 204 | 27.710 | −7.096 | 31.998 | 1.00 | 55.13 | | O |
| ATOM | 1077 | N | PRO G | 205 | 27.783 | −5.693 | 33.770 | 1.00 | 58.64 | | N |
| ATOM | 1078 | CA | PRO G | 205 | 29.236 | −5.775 | 33.842 | 1.00 | 52.19 | | C |
| ATOM | 1079 | CB | PRO G | 205 | 29.551 | −5.040 | 35.142 | 1.00 | 20.00 | | C |
| ATOM | 1080 | CG | PRO G | 205 | 28.379 | −5.337 | 36.014 | 1.00 | 20.00 | | C |
| ATOM | 1081 | CD | PRO G | 205 | 27.182 | −5.464 | 35.099 | 1.00 | 20.00 | | C |
| ATOM | 1082 | C | PRO G | 205 | 29.647 | −7.230 | 33.970 | 1.00 | 54.30 | | C |
| ATOM | 1083 | O | PRO G | 205 | 30.783 | −7.593 | 33.663 | 1.00 | 70.22 | | O |
| ATOM | 1084 | N | ILE G | 206 | 28.709 | −8.050 | 34.430 | 1.00 | 62.70 | | N |
| ATOM | 1085 | CA | ILE G | 206 | 28.925 | −9.480 | 34.557 | 1.00 | 68.19 | | C |
| ATOM | 1086 | CB | ILE G | 206 | 29.450 | −9.843 | 35.957 | 1.00 | 20.00 | | C |
| ATOM | 1087 | CG1 | ILE G | 206 | 30.853 | −9.269 | 36.164 | 1.00 | 20.00 | | C |
| ATOM | 1088 | CD1 | ILE G | 206 | 31.328 | −9.317 | 37.600 | 1.00 | 20.00 | | C |
| ATOM | 1089 | CG2 | ILE G | 206 | 29.454 | −11.352 | 36.151 | 1.00 | 20.00 | | C |
| ATOM | 1090 | C | ILE G | 206 | 27.618 | −10.223 | 34.304 | 1.00 | 62.35 | | C |
| ATOM | 1091 | O | ILE G | 206 | 26.548 | −9.772 | 34.715 | 1.00 | 67.16 | | O |
| ATOM | 1092 | N | VAL G | 207 | 27.702 | −11.314 | 33.552 | 1.00 | 52.22 | | N |
| ATOM | 1093 | CA | VAL G | 207 | 26.527 | −12.112 | 33.234 | 1.00 | 51.31 | | C |
| ATOM | 1094 | CB | VAL G | 207 | 26.414 | −12.378 | 31.722 | 1.00 | 20.00 | | C |
| ATOM | 1095 | CG1 | VAL G | 207 | 25.235 | −13.293 | 31.430 | 1.00 | 20.00 | | C |
| ATOM | 1096 | CG2 | VAL G | 207 | 26.279 | −11.066 | 30.963 | 1.00 | 20.00 | | C |
| ATOM | 1097 | C | VAL G | 207 | 26.546 | −13.438 | 33.985 | 1.00 | 62.09 | | C |
| ATOM | 1098 | O | VAL G | 207 | 27.257 | −14.369 | 33.605 | 1.00 | 58.88 | | O |
| ATOM | 1099 | N | ASN G | 208 | 25.828 | −13.484 | 35.102 | 1.00 | 71.72 | | N |
| ATOM | 1100 | CA | ASN G | 208 | 25.723 | −14.698 | 35.900 | 1.00 | 70.92 | | C |
| ATOM | 1101 | CB | ASN G | 208 | 25.837 | −14.370 | 37.390 | 1.00 | 20.00 | | C |
| ATOM | 1102 | CG | ASN G | 208 | 27.235 | −13.938 | 37.787 | 1.00 | 20.00 | | C |
| ATOM | 1103 | OD1 | ASN G | 208 | 28.216 | −14.286 | 37.129 | 1.00 | 20.00 | | O |
| ATOM | 1104 | ND2 | ASN G | 208 | 27.335 | −13.190 | 38.880 | 1.00 | 20.00 | | N |
| ATOM | 1105 | C | ASN G | 208 | 24.423 | −15.446 | 35.628 | 1.00 | 63.05 | | C |
| ATOM | 1106 | O | ASN G | 208 | 23.637 | −15.050 | 34.767 | 1.00 | 61.34 | | O |
| ATOM | 1107 | N | LYS G | 209 | 24.168 | −16.485 | 36.418 | 1.00 | 68.91 | | N |
| ATOM | 1108 | CA | LYS G | 209 | 23.006 | −17.341 | 36.212 | 1.00 | 59.11 | | C |
| ATOM | 1109 | CB | LYS G | 209 | 23.129 | −18.620 | 37.043 | 1.00 | 20.00 | | C |
| ATOM | 1110 | CG | LYS G | 209 | 24.239 | −19.552 | 36.588 | 1.00 | 20.00 | | C |
| ATOM | 1111 | CD | LYS G | 209 | 24.335 | −20.774 | 37.486 | 1.00 | 20.00 | | C |
| ATOM | 1112 | CE | LYS G | 209 | 25.423 | −21.724 | 37.012 | 1.00 | 20.00 | | C |
| ATOM | 1113 | NZ | LYS G | 209 | 25.548 | −22.912 | 37.901 | 1.00 | 20.00 | | N |
| ATOM | 1114 | C | LYS G | 209 | 21.696 | −16.628 | 36.539 | 1.00 | 50.69 | | C |
| ATOM | 1115 | O | LYS G | 209 | 20.616 | −17.121 | 36.212 | 1.00 | 51.01 | | O |
| ATOM | 1116 | N | GLN G | 210 | 21.792 | −15.499 | 37.235 | 1.00 | 44.68 | | N |
| ATOM | 1117 | CA | GLN G | 210 | 20.621 | −14.676 | 37.518 | 1.00 | 49.52 | | C |
| ATOM | 1118 | CB | GLN G | 210 | 20.828 | −13.874 | 38.806 | 1.00 | 20.00 | | C |
| ATOM | 1119 | CG | GLN G | 210 | 20.877 | −14.719 | 40.070 | 1.00 | 20.00 | | C |
| ATOM | 1120 | CD | GLN G | 210 | 21.149 | −13.893 | 41.313 | 1.00 | 20.00 | | C |
| ATOM | 1121 | OE1 | GLN G | 210 | 21.709 | −12.800 | 41.237 | 1.00 | 20.00 | | O |
| ATOM | 1122 | NE2 | GLN G | 210 | 20.755 | −14.417 | 42.469 | 1.00 | 20.00 | | N |
| ATOM | 1123 | C | GLN G | 210 | 20.336 | −13.730 | 36.356 | 1.00 | 62.08 | | C |
| ATOM | 1124 | O | GLN G | 210 | 19.271 | −13.785 | 35.741 | 1.00 | 80.43 | | O |
| ATOM | 1125 | N | SER G | 211 | 21.324 | −12.905 | 36.028 | 1.00 | 58.92 | | N |
| ATOM | 1126 | CA | SER G | 211 | 21.231 | −11.987 | 34.898 | 1.00 | 47.88 | | C |
| ATOM | 1127 | CB | SER G | 211 | 22.594 | −11.350 | 34.621 | 1.00 | 20.00 | | C |
| ATOM | 1128 | OG | SER G | 211 | 23.553 | −12.334 | 34.274 | 1.00 | 20.00 | | O |
| ATOM | 1129 | C | SER G | 211 | 20.722 | −12.689 | 33.642 | 1.00 | 57.32 | | C |
| ATOM | 1130 | O | SER G | 211 | 19.919 | −12.133 | 32.893 | 1.00 | 50.74 | | O |
| ATOM | 1131 | N | CYS G | 212 | 21.149 | −13.934 | 33.451 | 1.00 | 44.34 | | N |
| ATOM | 1132 | CA | CYS G | 212 | 20.890 | −14.659 | 32.210 | 1.00 | 57.52 | | C |
| ATOM | 1133 | CB | CYS G | 212 | 21.127 | −16.180 | 32.363 | 1.00 | 20.00 | | C |
| ATOM | 1134 | SG | CYS G | 212 | 22.836 | −16.682 | 32.889 | 1.00 | 20.00 | | S |
| ATOM | 1135 | C | CYS G | 212 | 19.541 | −14.314 | 31.540 | 1.00 | 60.91 | | C |
| ATOM | 1136 | O | CYS G | 212 | 19.536 | −13.729 | 30.460 | 1.00 | 52.17 | | O |
| ATOM | 1137 | N | SER G | 213 | 18.465 | −14.813 | 32.151 | 1.00 | 21.55 | | N |
| ATOM | 1138 | CA | SER G | 213 | 17.107 | −14.631 | 31.656 | 1.00 | 21.67 | | C |
| ATOM | 1139 | CB | SER G | 213 | 16.133 | −15.512 | 32.444 | 1.00 | 21.81 | | C |
| ATOM | 1140 | OG | SER G | 213 | 16.471 | −16.882 | 32.323 | 1.00 | 23.00 | | O |
| ATOM | 1141 | C | SER G | 213 | 16.633 | −13.188 | 31.682 | 1.00 | 21.45 | | C |
| ATOM | 1142 | O | SER G | 213 | 16.026 | −12.728 | 30.722 | 1.00 | 21.98 | | O |
| ATOM | 1143 | N | ILE G | 214 | 16.856 | −12.475 | 32.783 | 1.00 | 20.90 | | N |
| ATOM | 1144 | CA | ILE G | 214 | 16.585 | −11.050 | 32.758 | 1.00 | 20.45 | | C |
| ATOM | 1145 | CB | ILE G | 214 | 16.587 | −10.444 | 34.170 | 1.00 | 20.53 | | C |
| ATOM | 1146 | CG1 | ILE G | 214 | 15.498 | −11.091 | 35.029 | 1.00 | 20.46 | | C |
| ATOM | 1147 | CD1 | ILE G | 214 | 15.418 | −10.535 | 36.434 | 1.00 | 21.75 | | C |
| ATOM | 1148 | CG2 | ILE G | 214 | 16.393 | −8.937 | 34.103 | 1.00 | 20.34 | | C |
| ATOM | 1149 | C | ILE G | 214 | 17.686 | −10.409 | 31.926 | 1.00 | 20.15 | | C |
| ATOM | 1150 | O | ILE G | 214 | 17.435 | −9.671 | 30.968 | 1.00 | 19.91 | | O |
| ATOM | 1151 | N | SER G | 215 | 18.918 | −10.770 | 32.280 | 1.00 | 19.84 | | N |
| ATOM | 1152 | CA | SER G | 215 | 20.109 | −10.297 | 31.594 | 1.00 | 19.58 | | C |
| ATOM | 1153 | CB | SER G | 215 | 21.369 | −10.722 | 32.349 | 1.00 | 19.76 | | C |
| ATOM | 1154 | OG | SER G | 215 | 22.538 | −10.269 | 31.688 | 1.00 | 19.97 | | O |
| ATOM | 1155 | C | SER G | 215 | 20.132 | −10.852 | 30.189 | 1.00 | 19.34 | | C |
| ATOM | 1156 | O | SER G | 215 | 20.448 | −10.152 | 29.230 | 1.00 | 19.54 | | O |
| ATOM | 1157 | N | ASN G | 216 | 19.774 | −12.125 | 30.082 | 1.00 | 18.80 | | N |
| ATOM | 1158 | CA | ASN G | 216 | 19.738 | −12.791 | 28.798 | 1.00 | 18.36 | | C |
| ATOM | 1159 | CB | ASN G | 216 | 19.394 | −14.271 | 28.966 | 1.00 | 18.41 | | C |
| ATOM | 1160 | CG | ASN G | 216 | 20.426 | −15.020 | 29.786 | 1.00 | 19.55 | | C |
| ATOM | 1161 | OD1 | ASN G | 216 | 20.308 | −16.227 | 30.000 | 1.00 | 20.52 | | O |
| ATOM | 1162 | ND2 | ASN G | 216 | 21.445 | −14.306 | 30.250 | 1.00 | 20.43 | | N |
| ATOM | 1163 | C | ASN G | 216 | 18.708 | −12.107 | 27.927 | 1.00 | 17.78 | | C |
| ATOM | 1164 | O | ASN G | 216 | 18.930 | −11.905 | 26.744 | 1.00 | 17.91 | | O |
| ATOM | 1165 | N | ILE G | 217 | 17.578 | −11.750 | 28.527 | 1.00 | 17.02 | | N |
| ATOM | 1166 | CA | ILE G | 217 | 16.502 | −11.089 | 27.804 | 1.00 | 16.60 | | C |
| ATOM | 1167 | CB | ILE G | 217 | 15.247 | −10.916 | 28.680 | 1.00 | 16.46 | | C |
| ATOM | 1168 | CG1 | ILE G | 217 | 14.608 | −12.275 | 28.969 | 1.00 | 16.26 | | C |
| ATOM | 1169 | CD1 | ILE G | 217 | 13.360 | −12.195 | 29.822 | 1.00 | 16.44 | | C |
| ATOM | 1170 | CG2 | ILE G | 217 | 14.251 | −9.987 | 28.005 | 1.00 | 16.70 | | C |
| ATOM | 1171 | C | ILE G | 217 | 16.953 | −9.731 | 27.297 | 1.00 | 16.73 | | C |
| ATOM | 1172 | O | ILE G | 217 | 16.658 | −9.355 | 26.166 | 1.00 | 16.80 | | O |
| ATOM | 1173 | N | GLU G | 218 | 17.679 | −9.000 | 28.137 | 1.00 | 17.02 | | N |
| ATOM | 1174 | CA | GLU G | 218 | 18.177 | −7.692 | 27.741 | 1.00 | 17.70 | | C |
| ATOM | 1175 | CB | GLU G | 218 | 18.867 | −6.998 | 28.916 | 1.00 | 18.20 | | C |
| ATOM | 1176 | CG | GLU G | 218 | 17.955 | −6.748 | 30.106 | 1.00 | 21.31 | | C |
| ATOM | 1177 | CD | GLU G | 218 | 18.652 | −6.004 | 31.228 | 1.00 | 25.72 | | C |
| ATOM | 1178 | OE1 | GLU G | 218 | 18.016 | −5.781 | 32.279 | 1.00 | 26.96 | | O |
| ATOM | 1179 | OE2 | GLU G | 218 | 19.834 | −5.640 | 31.058 | 1.00 | 27.07 | | O |
| ATOM | 1180 | C | GLU G | 218 | 19.144 | −7.855 | 26.578 | 1.00 | 17.29 | | C |
| ATOM | 1181 | O | GLU G | 218 | 19.123 | −7.081 | 25.617 | 1.00 | 17.62 | | O |
| ATOM | 1182 | N | THR G | 219 | 19.983 | −8.882 | 26.667 | 1.00 | 16.62 | | N |
| ATOM | 1183 | CA | THR G | 219 | 20.964 | −9.150 | 25.630 | 1.00 | 15.95 | | C |
| ATOM | 1184 | CB | THR G | 219 | 21.879 | −10.328 | 26.007 | 1.00 | 15.95 | | C |
| ATOM | 1185 | OG1 | THR G | 219 | 22.582 | −10.021 | 27.218 | 1.00 | 15.63 | | O |
| ATOM | 1186 | CG2 | THR G | 219 | 22.884 | −10.597 | 24.897 | 1.00 | 15.97 | | C |
| ATOM | 1187 | C | THR G | 219 | 20.250 | −9.470 | 24.330 | 1.00 | 15.64 | | C |
| ATOM | 1188 | O | THR G | 219 | 20.656 | −9.022 | 23.267 | 1.00 | 15.93 | | O |
| ATOM | 1189 | N | VAL G | 220 | 19.179 | −10.248 | 24.431 | 1.00 | 14.98 | | N |
| ATOM | 1190 | CA | VAL G | 220 | 18.390 | −10.644 | 23.278 | 1.00 | 14.51 | | C |
| ATOM | 1191 | CB | VAL G | 220 | 17.285 | −11.644 | 23.665 | 1.00 | 14.29 | | C |
| ATOM | 1192 | CG1 | VAL G | 220 | 16.474 | −12.037 | 22.440 | 1.00 | 14.24 | | C |
| ATOM | 1193 | CG2 | VAL G | 220 | 17.890 | −12.871 | 24.329 | 1.00 | 14.10 | | C |
| ATOM | 1194 | C | VAL G | 220 | 17.759 | −9.424 | 22.636 | 1.00 | 14.64 | | C |
| ATOM | 1195 | O | VAL G | 220 | 17.718 | −9.315 | 21.420 | 1.00 | 14.79 | | O |
| ATOM | 1196 | N | ILE G | 221 | 17.264 | −8.510 | 23.463 | 1.00 | 14.43 | | N |
| ATOM | 1197 | CA | ILE G | 221 | 16.658 | −7.287 | 22.959 | 1.00 | 14.36 | | C |
| ATOM | 1198 | CB | ILE G | 221 | 16.046 | −6.448 | 24.095 | 1.00 | 14.03 | | C |
| ATOM | 1199 | CG1 | ILE G | 221 | 14.955 | −7.243 | 24.816 | 1.00 | 13.72 | | C |
| ATOM | 1200 | CD1 | ILE G | 221 | 13.820 | −7.679 | 23.914 | 1.00 | 14.17 | | C |
| ATOM | 1201 | CG2 | ILE G | 221 | 15.487 | −5.142 | 23.550 | 1.00 | 13.91 | | C |
| ATOM | 1202 | C | ILE G | 221 | 17.700 | −6.459 | 22.222 | 1.00 | 14.82 | | C |
| ATOM | 1203 | O | ILE G | 221 | 17.427 | −5.902 | 21.158 | 1.00 | 14.82 | | O |
| ATOM | 1204 | N | GLU G | 222 | 18.901 | −6.390 | 22.788 | 1.00 | 15.51 | | N |
| ATOM | 1205 | CA | GLU G | 222 | 19.983 | −5.640 | 22.165 | 1.00 | 16.52 | | C |
| ATOM | 1206 | CB | GLU G | 222 | 21.211 | −5.601 | 23.076 | 1.00 | 16.80 | | C |
| ATOM | 1207 | CG | GLU G | 222 | 20.971 | −4.899 | 24.403 | 1.00 | 19.14 | | C |
| ATOM | 1208 | CD | GLU G | 222 | 22.231 | −4.780 | 25.236 | 1.00 | 22.62 | | C |
| ATOM | 1209 | OE1 | GLU G | 222 | 22.152 | −4.246 | 26.362 | 1.00 | 24.35 | | O |
| ATOM | 1210 | OE2 | GLU G | 222 | 23.301 | −5.219 | 24.765 | 1.00 | 23.98 | | O |
| ATOM | 1211 | C | GLU G | 222 | 20.335 | −6.269 | 20.825 | 1.00 | 16.65 | | C |
| ATOM | 1212 | O | GLU G | 222 | 20.599 | −5.574 | 19.842 | 1.00 | 16.95 | | O |
| ATOM | 1213 | N | PHE G | 223 | 20.339 | −7.597 | 20.802 | 1.00 | 16.70 | | N |
| ATOM | 1214 | CA | PHE G | 223 | 20.652 | −8.350 | 19.603 | 1.00 | 16.95 | | C |
| ATOM | 1215 | CB | PHE G | 223 | 20.705 | −9.848 | 19.904 | 1.00 | 16.85 | | C |
| ATOM | 1216 | CG | PHE G | 223 | 21.104 | −10.689 | 18.724 | 1.00 | 17.23 | | C |
| ATOM | 1217 | CD1 | PHE G | 223 | 22.438 | −10.954 | 18.465 | 1.00 | 17.98 | | C |
| ATOM | 1218 | CE1 | PHE G | 223 | 22.808 | −11.727 | 17.381 | 1.00 | 18.28 | | C |
| ATOM | 1219 | CZ | PHE G | 223 | 21.842 | −12.243 | 16.541 | 1.00 | 18.19 | | C |
| ATOM | 1220 | CE2 | PHE G | 223 | 20.508 | −11.986 | 16.788 | 1.00 | 18.14 | | C |
| ATOM | 1221 | CD2 | PHE G | 223 | 20.144 | −11.213 | 17.874 | 1.00 | 17.75 | | C |
| ATOM | 1222 | C | PHE G | 223 | 19.610 | −8.066 | 18.540 | 1.00 | 17.28 | | C |
| ATOM | 1223 | O | PHE G | 223 | 19.945 | −7.907 | 17.382 | 1.00 | 17.17 | | O |
| ATOM | 1224 | N | GLN G | 224 | 18.348 | −8.003 | 18.948 | 1.00 | 17.62 | | N |
| ATOM | 1225 | CA | GLN G | 224 | 17.243 | −7.712 | 18.044 | 1.00 | 18.16 | | C |
| ATOM | 1226 | CB | GLN G | 224 | 15.898 | −7.868 | 18.757 | 1.00 | 18.51 | | C |
| ATOM | 1227 | CG | GLN G | 224 | 15.586 | −9.296 | 19.178 | 1.00 | 20.06 | | C |
| ATOM | 1228 | CD | GLN G | 224 | 14.158 | −9.465 | 19.658 | 1.00 | 22.03 | | C |
| ATOM | 1229 | OE1 | GLN G | 224 | 13.769 | −10.539 | 20.117 | 1.00 | 22.15 | | O |
| ATOM | 1230 | NE2 | GLN G | 224 | 13.368 | −8.403 | 19.552 | 1.00 | 22.11 | | N |
| ATOM | 1231 | C | GLN G | 224 | 17.387 | −6.309 | 17.475 | 1.00 | 18.04 | | C |
| ATOM | 1232 | O | GLN G | 224 | 17.090 | −6.068 | 16.306 | 1.00 | 18.17 | | O |
| ATOM | 1233 | N | GLN G | 225 | 17.819 | −5.382 | 18.323 | 1.00 | 17.90 | | N |
| ATOM | 1234 | CA | GLN G | 225 | 18.051 | −4.012 | 17.894 | 1.00 | 17.95 | | C |
| ATOM | 1235 | CB | GLN G | 225 | 18.389 | −3.120 | 19.090 | 1.00 | 18.13 | | C |
| ATOM | 1236 | CG | GLN G | 225 | 17.267 | −3.006 | 20.109 | 1.00 | 19.04 | | C |
| ATOM | 1237 | CD | GLN G | 225 | 17.588 | −2.034 | 21.227 | 1.00 | 21.22 | | C |
| ATOM | 1238 | OE1 | GLN G | 225 | 18.661 | −1.431 | 21.252 | 1.00 | 22.55 | | O |
| ATOM | 1239 | NE2 | GLN G | 225 | 16.656 | −1.876 | 22.159 | 1.00 | 22.48 | | N |
| ATOM | 1240 | C | GLN G | 225 | 19.168 | −3.958 | 16.855 | 1.00 | 17.84 | | C |
| ATOM | 1241 | O | GLN G | 225 | 19.061 | −3.239 | 15.861 | 1.00 | 17.71 | | O |
| ATOM | 1242 | N | LYS G | 226 | 20.235 | −4.724 | 17.077 | 1.00 | 17.95 | | N |
| ATOM | 1243 | CA | LYS G | 226 | 21.328 | −4.762 | 16.112 | 1.00 | 18.00 | | C |
| ATOM | 1244 | CB | LYS G | 226 | 22.486 | −5.613 | 16.634 | 1.00 | 18.21 | | C |
| ATOM | 1245 | CG | LYS G | 226 | 23.157 | −5.048 | 17.875 | 1.00 | 18.50 | | C |
| ATOM | 1246 | CD | LYS G | 226 | 24.365 | −5.878 | 18.279 | 1.00 | 19.87 | | C |
| ATOM | 1247 | CE | LYS G | 226 | 25.066 | −5.278 | 19.487 | 1.00 | 21.36 | | C |
| ATOM | 1248 | NZ | LYS G | 226 | 26.270 | −6.061 | 19.878 | 1.00 | 21.83 | | N |
| ATOM | 1249 | C | LYS G | 226 | 20.772 | −5.345 | 14.826 | 1.00 | 17.84 | | C |
| ATOM | 1250 | O | LYS G | 226 | 21.062 | −4.880 | 13.723 | 1.00 | 17.94 | | O |
| ATOM | 1251 | N | ASN G | 227 | 19.954 | −6.374 | 15.002 | 1.00 | 17.70 | | N |
| ATOM | 1252 | CA | ASN G | 227 | 19.238 | −7.032 | 13.932 | 1.00 | 17.66 | | C |
| ATOM | 1253 | CB | ASN G | 227 | 18.471 | −8.244 | 14.459 | 1.00 | 17.36 | | C |
| ATOM | 1254 | CG | ASN G | 227 | 19.384 | −9.296 | 15.057 | 1.00 | 17.40 | | C |
| ATOM | 1255 | OD1 | ASN G | 227 | 18.929 | −10.350 | 15.502 | 1.00 | 17.84 | | O |
| ATOM | 1256 | ND2 | ASN G | 227 | 20.682 | −9.015 | 15.070 | 1.00 | 17.22 | | N |
| ATOM | 1257 | C | ASN G | 227 | 18.287 | −6.028 | 13.328 | 1.00 | 17.76 | | C |
| ATOM | 1258 | O | ASN G | 227 | 18.159 | −5.956 | 12.106 | 1.00 | 17.92 | | O |
| ATOM | 1259 | N | ASN G | 228 | 17.627 | −5.226 | 14.164 | 1.00 | 17.78 | | N |
| ATOM | 1260 | CA | ASN G | 228 | 16.800 | −4.172 | 13.548 | 1.00 | 17.59 | | C |
| ATOM | 1261 | CB | ASN G | 228 | 15.958 | −3.344 | 14.528 | 1.00 | 17.89 | | C |
| ATOM | 1262 | CG | ASN G | 228 | 15.101 | −2.277 | 13.809 | 1.00 | 18.43 | | C |
| ATOM | 1263 | OD1 | ASN G | 228 | 15.506 | −1.119 | 13.688 | 1.00 | 18.74 | | O |
| ATOM | 1264 | ND2 | ASN G | 228 | 13.998 | −2.715 | 13.205 | 1.00 | 18.61 | | N |
| ATOM | 1265 | C | ASN G | 228 | 17.603 | −3.255 | 12.662 | 1.00 | 17.09 | | C |
| ATOM | 1266 | O | ASN G | 228 | 17.657 | −3.466 | 11.456 | 1.00 | 17.17 | | O |
| ATOM | 1267 | N | ARG G | 229 | 18.197 | −2.211 | 13.224 | 1.00 | 16.33 | | N |
| ATOM | 1268 | CA | ARG G | 229 | 19.046 | −1.400 | 12.381 | 1.00 | 15.76 | | C |
| ATOM | 1269 | CB | ARG G | 229 | 19.957 | −0.486 | 13.184 | 1.00 | 15.70 | | C |
| ATOM | 1270 | CG | ARG G | 229 | 20.207 | 0.822 | 12.468 | 1.00 | 14.99 | | C |
| ATOM | 1271 | CD | ARG G | 229 | 21.391 | 1.555 | 13.041 | 1.00 | 15.33 | | C |
| ATOM | 1272 | NE | ARG G | 229 | 22.116 | 2.303 | 12.021 | 1.00 | 15.59 | | N |
| ATOM | 1273 | CZ | ARG G | 229 | 23.182 | 3.053 | 12.276 | 1.00 | 15.85 | | C |
| ATOM | 1274 | NH1 | ARG G | 229 | 23.593 | 3.214 | 13.525 | 1.00 | 16.20 | | N |
| ATOM | 1275 | NH2 | ARG G | 229 | 23.859 | 3.614 | 11.285 | 1.00 | 16.63 | | N |
| ATOM | 1276 | C | ARG G | 229 | 19.863 | −2.343 | 11.516 | 1.00 | 15.62 | | C |
| ATOM | 1277 | O | ARG G | 229 | 19.671 | −2.402 | 10.304 | 1.00 | 15.73 | | O |
| ATOM | 1278 | N | LEU G | 230 | 20.612 | −3.225 | 12.168 | 1.00 | 15.46 | | N |
| ATOM | 1279 | CA | LEU G | 230 | 21.409 | −4.212 | 11.454 | 1.00 | 15.22 | | C |
| ATOM | 1280 | CB | LEU G | 230 | 22.161 | −5.113 | 12.432 | 1.00 | 15.08 | | C |
| ATOM | 1281 | CG | LEU G | 230 | 23.055 | −6.187 | 11.808 | 1.00 | 14.70 | | C |
| ATOM | 1282 | CD1 | LEU G | 230 | 24.375 | −5.579 | 11.364 | 1.00 | 15.19 | | C |
| ATOM | 1283 | CD2 | LEU G | 230 | 23.300 | −7.324 | 12.791 | 1.00 | 15.46 | | C |
| ATOM | 1284 | C | LEU G | 230 | 20.565 | −5.055 | 10.501 | 1.00 | 15.31 | | C |
| ATOM | 1285 | O | LEU G | 230 | 20.963 | −5.294 | 9.362 | 1.00 | 15.46 | | O |
| ATOM | 1286 | N | LEU G | 231 | 19.444 | −5.573 | 10.997 | 1.00 | 15.48 | | N |
| ATOM | 1287 | CA | LEU G | 231 | 18.581 | −6.442 | 10.197 | 1.00 | 15.75 | | C |
| ATOM | 1288 | CB | LEU G | 231 | 17.308 | −6.793 | 10.967 | 1.00 | 15.90 | | C |
| ATOM | 1289 | CG | LEU G | 231 | 17.482 | −7.640 | 12.226 | 1.00 | 16.63 | | C |
| ATOM | 1290 | CD1 | LEU G | 231 | 16.128 | −8.049 | 12.772 | 1.00 | 18.45 | | C |
| ATOM | 1291 | CD2 | LEU G | 231 | 18.348 | −8.858 | 11.954 | 1.00 | 16.67 | | C |
| ATOM | 1292 | C | LEU G | 231 | 18.210 | −5.785 | 8.872 | 1.00 | 15.76 | | C |
| ATOM | 1293 | O | LEU G | 231 | 18.203 | −6.432 | 7.824 | 1.00 | 15.84 | | O |
| ATOM | 1294 | N | GLU G | 232 | 17.824 | −4.516 | 8.943 | 1.00 | 15.71 | | N |
| ATOM | 1295 | CA | GLU G | 232 | 17.298 | −3.811 | 7.786 | 1.00 | 15.93 | | C |
| ATOM | 1296 | CB | GLU G | 232 | 16.537 | −2.565 | 8.229 | 1.00 | 16.15 | | C |
| ATOM | 1297 | CG | GLU G | 232 | 15.461 | −2.846 | 9.254 | 1.00 | 18.59 | | C |
| ATOM | 1298 | CD | GLU G | 232 | 14.077 | −2.874 | 8.644 | 1.00 | 21.47 | | C |
| ATOM | 1299 | OE1 | GLU G | 232 | 13.964 | −3.077 | 7.415 | 1.00 | 22.14 | | O |
| ATOM | 1300 | OE2 | GLU G | 232 | 13.100 | −2.664 | 9.393 | 1.00 | 23.34 | | O |
| ATOM | 1301 | C | GLU G | 232 | 18.399 | −3.431 | 6.806 | 1.00 | 15.45 | | C |
| ATOM | 1302 | O | GLU G | 232 | 18.169 | −3.373 | 5.601 | 1.00 | 15.95 | | O |
| ATOM | 1303 | N | ILE G | 233 | 19.594 | −3.169 | 7.323 | 1.00 | 14.54 | | N |
| ATOM | 1304 | CA | ILE G | 233 | 20.774 | −3.128 | 6.476 | 1.00 | 13.69 | | C |
| ATOM | 1305 | CB | ILE G | 233 | 22.049 | −2.830 | 7.281 | 1.00 | 13.63 | | C |
| ATOM | 1306 | CG1 | ILE G | 233 | 21.823 | −1.640 | 8.211 | 1.00 | 13.58 | | C |
| ATOM | 1307 | CD1 | ILE G | 233 | 23.101 | −0.917 | 8.578 | 1.00 | 13.55 | | C |
| ATOM | 1308 | CG2 | ILE G | 233 | 23.215 | −2.543 | 6.348 | 1.00 | 13.88 | | C |
| ATOM | 1309 | C | ILE G | 233 | 20.927 | −4.450 | 5.736 | 1.00 | 13.28 | | C |
| ATOM | 1310 | O | ILE G | 233 | 20.979 | −4.473 | 4.508 | 1.00 | 13.04 | | O |
| ATOM | 1311 | N | THR G | 234 | 20.758 | −5.546 | 6.469 | 1.00 | 13.18 | | N |
| ATOM | 1312 | CA | THR G | 234 | 21.038 | −6.878 | 5.940 | 1.00 | 13.39 | | C |
| ATOM | 1313 | CB | THR G | 234 | 20.839 | −7.965 | 7.015 | 1.00 | 13.21 | | C |
| ATOM | 1314 | OG1 | THR G | 234 | 21.845 | −7.836 | 8.028 | 1.00 | 13.15 | | O |
| ATOM | 1315 | CG2 | THR G | 234 | 20.916 | −9.351 | 6.394 | 1.00 | 13.22 | | C |
| ATOM | 1316 | C | THR G | 234 | 20.146 | −7.204 | 4.748 | 1.00 | 13.76 | | C |
| ATOM | 1317 | O | THR G | 234 | 20.606 | −7.766 | 3.753 | 1.00 | 13.95 | | O |
| ATOM | 1318 | N | ARG G | 235 | 18.849 | −6.966 | 4.911 | 1.00 | 14.26 | | N |
| ATOM | 1319 | CA | ARG G | 235 | 17.891 | −7.151 | 3.826 | 1.00 | 14.92 | | C |
| ATOM | 1320 | CB | ARG G | 235 | 16.463 | −6.925 | 4.328 | 1.00 | 15.30 | | C |
| ATOM | 1321 | CG | ARG G | 235 | 15.771 | −5.712 | 3.722 | 1.00 | 16.27 | | C |
| ATOM | 1322 | CD | ARG G | 235 | 14.867 | −5.020 | 4.735 | 1.00 | 18.52 | | C |
| ATOM | 1323 | NE | ARG G | 235 | 14.345 | −5.948 | 5.734 | 1.00 | 17.86 | | N |
| ATOM | 1324 | CZ | ARG G | 235 | 13.438 | −6.884 | 5.478 | 1.00 | 16.91 | | C |
| ATOM | 1325 | NH1 | ARG G | 235 | 12.970 | −7.035 | 4.248 | 1.00 | 16.59 | | N |
| ATOM | 1326 | NH2 | ARG G | 235 | 13.018 | −7.687 | 6.444 | 1.00 | 16.86 | | N |
| ATOM | 1327 | C | ARG G | 235 | 18.192 | −6.211 | 2.665 | 1.00 | 15.05 | | C |
| ATOM | 1328 | O | ARG G | 235 | 18.292 | −6.640 | 1.517 | 1.00 | 15.15 | | O |
| ATOM | 1329 | N | GLU G | 236 | 18.386 | −4.935 | 2.984 | 1.00 | 15.18 | | N |
| ATOM | 1330 | CA | GLU G | 236 | 18.751 | −3.930 | 1.992 | 1.00 | 15.24 | | C |
| ATOM | 1331 | CB | GLU G | 236 | 19.179 | −2.636 | 2.687 | 1.00 | 15.69 | | C |
| ATOM | 1332 | CG | GLU G | 236 | 18.875 | −1.369 | 1.904 | 1.00 | 18.22 | | C |
| ATOM | 1333 | CD | GLU G | 236 | 19.004 | −0.115 | 2.750 | 1.00 | 21.59 | | C |
| ATOM | 1334 | OE1 | GLU G | 236 | 19.395 | 0.937 | 2.199 | 1.00 | 24.41 | | O |
| ATOM | 1335 | OE2 | GLU G | 236 | 18.660 | −0.168 | 3.953 | 1.00 | 21.59 | | O |
| ATOM | 1336 | C | GLU G | 236 | 19.870 | −4.421 | 1.077 | 1.00 | 14.61 | | C |
| ATOM | 1337 | O | GLU G | 236 | 19.746 | −4.377 | −0.147 | 1.00 | 14.82 | | O |
| ATOM | 1338 | N | PHE G | 237 | 20.963 | −4.883 | 1.675 | 1.00 | 14.05 | | N |
| ATOM | 1339 | CA | PHE G | 237 | 22.110 | −5.358 | 0.907 | 1.00 | 13.84 | | C |
| ATOM | 1340 | CB | PHE G | 237 | 23.285 | −5.682 | 1.836 | 1.00 | 14.04 | | C |
| ATOM | 1341 | CG | PHE G | 237 | 24.161 | −4.500 | 2.143 | 1.00 | 14.84 | | C |
| ATOM | 1342 | CD1 | PHE G | 237 | 24.412 | −4.131 | 3.454 | 1.00 | 15.30 | | C |
| ATOM | 1343 | CE1 | PHE G | 237 | 25.268 | −3.087 | 3.743 | 1.00 | 15.15 | | C |
| ATOM | 1344 | CZ | PHE G | 237 | 25.871 | −2.389 | 2.717 | 1.00 | 15.19 | | C |
| ATOM | 1345 | CE2 | PHE G | 237 | 25.645 | −2.760 | 1.407 | 1.00 | 15.02 | | C |
| ATOM | 1346 | CD2 | PHE G | 237 | 24.796 | −3.809 | 1.125 | 1.00 | 15.03 | | C |
| ATOM | 1347 | C | PHE G | 237 | 21.741 | −6.586 | 0.078 | 1.00 | 13.51 | | C |
| ATOM | 1348 | O | PHE G | 237 | 22.027 | −6.652 | −1.117 | 1.00 | 13.43 | | O |
| ATOM | 1349 | N | SER G | 238 | 21.092 | −7.551 | 0.721 | 1.00 | 13.11 | | N |
| ATOM | 1350 | CA | SER G | 238 | 20.669 | −8.772 | 0.049 | 1.00 | 12.82 | | C |
| ATOM | 1351 | CB | SER G | 238 | 19.929 | −9.688 | 1.021 | 1.00 | 12.86 | | C |
| ATOM | 1352 | OG | SER G | 238 | 20.730 | −9.975 | 2.151 | 1.00 | 12.76 | | O |
| ATOM | 1353 | C | SER G | 238 | 19.770 | −8.444 | −1.131 | 1.00 | 12.69 | | C |
| ATOM | 1354 | O | SER G | 238 | 19.749 | −9.165 | −2.128 | 1.00 | 12.63 | | O |
| ATOM | 1355 | N | VAL G | 239 | 19.024 | −7.352 | −1.010 | 1.00 | 12.59 | | N |
| ATOM | 1356 | CA | VAL G | 239 | 18.143 | −6.916 | −2.082 | 1.00 | 12.80 | | C |
| ATOM | 1357 | CB | VAL G | 239 | 17.045 | −5.961 | −1.578 | 1.00 | 12.71 | | C |
| ATOM | 1358 | CG1 | VAL G | 239 | 16.215 | −5.451 | −2.747 | 1.00 | 13.63 | | C |
| ATOM | 1359 | CG2 | VAL G | 239 | 16.157 | −6.658 | −0.562 | 1.00 | 13.57 | | C |
| ATOM | 1360 | C | VAL G | 239 | 18.906 | −6.242 | −3.214 | 1.00 | 12.94 | | C |
| ATOM | 1361 | O | VAL G | 239 | 18.337 | −5.980 | −4.273 | 1.00 | 13.10 | | O |
| ATOM | 1362 | N | ASN G | 240 | 20.173 | −5.913 | −2.981 | 1.00 | 4.15 | | N |
| ATOM | 1363 | CA | ASN G | 240 | 20.892 | −5.047 | −3.909 | 1.00 | 4.33 | | C |
| ATOM | 1364 | CB | ASN G | 240 | 21.377 | −3.779 | −3.214 | 1.00 | 4.59 | | C |
| ATOM | 1365 | CG | ASN G | 240 | 20.260 | −2.790 | −2.978 | 1.00 | 5.36 | | C |
| ATOM | 1366 | OD1 | ASN G | 240 | 19.681 | −2.263 | −3.926 | 1.00 | 6.27 | | O |
| ATOM | 1367 | ND2 | ASN G | 240 | 19.828 | −2.683 | −1.727 | 1.00 | 7.15 | | N |
| ATOM | 1368 | C | ASN G | 240 | 22.025 | −5.707 | −4.689 | 1.00 | 4.19 | | C |
| ATOM | 1369 | O | ASN G | 240 | 22.565 | −5.115 | −5.625 | 1.00 | 4.19 | | O |
| ATOM | 1370 | N | ALA G | 241 | 22.299 | −6.970 | −4.382 | 1.00 | 3.93 | | N |
| ATOM | 1371 | CA | ALA G | 241 | 23.459 | −7.657 | −4.941 | 1.00 | 3.90 | | C |
| ATOM | 1372 | CB | ALA G | 241 | 23.179 | −8.102 | −6.368 | 1.00 | 3.86 | | C |
| ATOM | 1373 | C | ALA G | 241 | 24.702 | −6.772 | −4.887 | 1.00 | 4.22 | | C |
| ATOM | 1374 | O | ALA G | 241 | 25.347 | −6.532 | −5.907 | 1.00 | 4.47 | | O |
| ATOM | 1375 | N | GLY G | 242 | 24.955 | −6.190 | −3.719 | 1.00 | 4.53 | | N |
| ATOM | 1376 | CA | GLY G | 242 | 26.289 | −5.717 | −3.368 | 1.00 | 4.65 | | C |
| ATOM | 1377 | C | GLY G | 242 | 26.609 | −4.323 | −3.872 | 1.00 | 4.67 | | C |
| ATOM | 1378 | O | GLY G | 242 | 27.770 | −3.913 | −3.869 | 1.00 | 5.27 | | O |
| ATOM | 1379 | N | VAL G | 243 | 25.577 | −3.570 | −4.246 | 1.00 | 4.37 | | N |
| ATOM | 1380 | CA | VAL G | 243 | 25.750 | −2.172 | −4.646 | 1.00 | 4.32 | | C |
| ATOM | 1381 | CB | VAL G | 243 | 26.048 | −2.039 | −6.153 | 1.00 | 3.99 | | C |
| ATOM | 1382 | CG1 | VAL G | 243 | 26.962 | −0.845 | −6.414 | 1.00 | 4.22 | | C |
| ATOM | 1383 | CG2 | VAL G | 243 | 26.649 | −3.327 | −6.699 | 1.00 | 3.85 | | C |
| ATOM | 1384 | C | VAL G | 243 | 24.510 | −1.342 | −4.335 | 1.00 | 4.61 | | C |
| ATOM | 1385 | O | VAL G | 243 | 23.394 | −1.734 | −4.673 | 1.00 | 4.89 | | O |
| ATOM | 1386 | N | THR G | 244 | 24.722 | −0.145 | −3.801 | 1.00 | 4.95 | | N |
| ATOM | 1387 | CA | THR G | 244 | 23.615 | 0.714 | −3.408 | 1.00 | 5.71 | | C |
| ATOM | 1388 | CB | THR G | 244 | 23.475 | 0.801 | −1.879 | 1.00 | 6.11 | | C |
| ATOM | 1389 | OG1 | THR G | 244 | 24.257 | −0.228 | −1.260 | 1.00 | 7.17 | | O |
| ATOM | 1390 | CG2 | THR G | 244 | 22.028 | 0.651 | −1.475 | 1.00 | 7.55 | | C |
| ATOM | 1391 | C | THR G | 244 | 23.802 | 2.114 | −3.967 | 1.00 | 5.61 | | C |
| ATOM | 1392 | O | THR G | 244 | 24.927 | 2.590 | −4.117 | 1.00 | 5.85 | | O |
| ATOM | 1393 | N | THR G | 245 | 22.695 | 2.794 | −4.223 | 1.00 | 5.53 | | N |
| ATOM | 1394 | CA | THR G | 245 | 22.677 | 4.241 | −4.120 | 1.00 | 5.81 | | C |
| ATOM | 1395 | CB | THR G | 245 | 22.461 | 4.903 | −5.490 | 1.00 | 6.13 | | C |
| ATOM | 1396 | OG1 | THR G | 245 | 23.555 | 4.575 | −6.355 | 1.00 | 8.11 | | O |
| ATOM | 1397 | CG2 | THR G | 245 | 22.394 | 6.408 | −5.336 | 1.00 | 6.63 | | C |
| ATOM | 1398 | C | THR G | 245 | 21.609 | 4.679 | −3.126 | 1.00 | 5.73 | | C |
| ATOM | 1399 | O | THR G | 245 | 20.778 | 3.869 | −2.718 | 1.00 | 5.86 | | O |
| ATOM | 1400 | N | PRO G | 246 | 21.521 | 5.993 | −2.867 | 1.00 | 5.68 | | N |
| ATOM | 1401 | CA | PRO G | 246 | 21.893 | 6.537 | −1.574 | 1.00 | 5.11 | | C |
| ATOM | 1402 | CB | PRO G | 246 | 20.537 | 6.719 | −0.893 | 1.00 | 5.19 | | C |
| ATOM | 1403 | CG | PRO G | 246 | 19.582 | 7.023 | −2.060 | 1.00 | 5.90 | | C |
| ATOM | 1404 | CD | PRO G | 246 | 20.305 | 6.660 | −3.353 | 1.00 | 6.36 | | C |
| ATOM | 1405 | C | PRO G | 246 | 22.831 | 5.666 | −0.743 | 1.00 | 4.85 | | C |
| ATOM | 1406 | O | PRO G | 246 | 22.663 | 4.447 | −0.677 | 1.00 | 5.25 | | O |
| ATOM | 1407 | N | VAL G | 247 | 23.900 | 6.281 | −0.249 | 1.00 | 4.35 | | N |
| ATOM | 1408 | CA | VAL G | 247 | 24.742 | 5.647 | 0.749 | 1.00 | 3.82 | | C |
| ATOM | 1409 | CB | VAL G | 247 | 26.177 | 6.196 | 0.717 | 1.00 | 3.52 | | C |
| ATOM | 1410 | CG1 | VAL G | 247 | 27.082 | 5.352 | 1.597 | 1.00 | 3.62 | | C |
| ATOM | 1411 | CG2 | VAL G | 247 | 26.697 | 6.207 | −0.707 | 1.00 | 3.60 | | C |
| ATOM | 1412 | C | VAL G | 247 | 24.134 | 5.824 | 2.131 | 1.00 | 3.69 | | C |
| ATOM | 1413 | O | VAL G | 247 | 24.038 | 6.942 | 2.637 | 1.00 | 3.36 | | O |
| ATOM | 1414 | N | SER G | 248 | 23.471 | 4.769 | 2.588 | 1.00 | 3.72 | | N |
| ATOM | 1415 | CA | SER G | 248 | 22.676 | 4.824 | 3.803 | 1.00 | 3.96 | | C |
| ATOM | 1416 | CB | SER G | 248 | 22.187 | 3.427 | 4.172 | 1.00 | 4.07 | | C |
| ATOM | 1417 | OG | SER G | 248 | 23.188 | 2.722 | 4.884 | 1.00 | 4.13 | | O |
| ATOM | 1418 | C | SER G | 248 | 23.484 | 5.392 | 4.959 | 1.00 | 4.35 | | C |
| ATOM | 1419 | O | SER G | 248 | 24.697 | 5.201 | 5.026 | 1.00 | 4.36 | | O |
| ATOM | 1420 | N | THR G | 249 | 22.776 | 5.880 | 5.972 | 1.00 | 4.53 | | N |
| ATOM | 1421 | CA | THR G | 249 | 23.405 | 6.259 | 7.226 | 1.00 | 4.61 | | C |
| ATOM | 1422 | CB | THR G | 249 | 22.408 | 6.945 | 8.175 | 1.00 | 4.70 | | C |
| ATOM | 1423 | OG1 | THR G | 249 | 21.562 | 5.959 | 8.778 | 1.00 | 5.42 | | O |
| ATOM | 1424 | CG2 | THR G | 249 | 21.553 | 7.945 | 7.412 | 1.00 | 4.88 | | C |
| ATOM | 1425 | C | THR G | 249 | 24.026 | 5.054 | 7.925 | 1.00 | 4.47 | | C |
| ATOM | 1426 | O | THR G | 249 | 24.830 | 5.210 | 8.844 | 1.00 | 4.74 | | O |
| ATOM | 1427 | N | TYR G | 250 | 23.694 | 3.855 | 7.454 | 1.00 | 4.32 | | N |
| ATOM | 1428 | CA | TYR G | 250 | 24.325 | 2.641 | 7.968 | 1.00 | 4.42 | | C |
| ATOM | 1429 | CB | TYR G | 250 | 23.393 | 1.435 | 7.845 | 1.00 | 4.91 | | C |
| ATOM | 1430 | CG | TYR G | 250 | 22.028 | 1.638 | 8.462 | 1.00 | 6.81 | | C |
| ATOM | 1431 | CD1 | TYR G | 250 | 20.881 | 1.242 | 7.790 | 1.00 | 8.89 | | C |
| ATOM | 1432 | CE1 | TYR G | 250 | 19.631 | 1.390 | 8.359 | 1.00 | 10.73 | | C |
| ATOM | 1433 | CZ | TYR G | 250 | 19.513 | 1.941 | 9.622 | 1.00 | 12.28 | | C |
| ATOM | 1434 | OH | TYR G | 250 | 18.265 | 2.135 | 10.172 | 1.00 | 12.24 | | O |
| ATOM | 1435 | CE2 | TYR G | 250 | 20.640 | 2.325 | 10.320 | 1.00 | 12.15 | | C |
| ATOM | 1436 | CD2 | TYR G | 250 | 21.890 | 2.148 | 9.749 | 1.00 | 9.81 | | C |
| ATOM | 1437 | C | TYR G | 250 | 25.656 | 2.342 | 7.285 | 1.00 | 4.21 | | C |
| ATOM | 1438 | O | TYR G | 250 | 26.539 | 1.730 | 7.886 | 1.00 | 4.20 | | O |
| ATOM | 1439 | N | MET G | 251 | 25.766 | 2.697 | 6.007 | 1.00 | 4.14 | | N |
| ATOM | 1440 | CA | MET G | 251 | 27.045 | 2.616 | 5.300 | 1.00 | 4.15 | | C |
| ATOM | 1441 | CB | MET G | 251 | 26.852 | 2.798 | 3.790 | 1.00 | 4.14 | | C |
| ATOM | 1442 | CG | MET G | 251 | 26.019 | 1.722 | 3.123 | 1.00 | 4.51 | | C |
| ATOM | 1443 | SD | MET G | 251 | 26.772 | 0.087 | 3.219 | 1.00 | 3.81 | | S |
| ATOM | 1444 | CE | MET G | 251 | 25.773 | −0.814 | 2.036 | 1.00 | 4.55 | | C |
| ATOM | 1445 | C | MET G | 251 | 27.996 | 3.686 | 5.816 | 1.00 | 4.13 | | C |
| ATOM | 1446 | O | MET G | 251 | 29.178 | 3.427 | 6.043 | 1.00 | 4.24 | | O |
| ATOM | 1447 | N | LEU G | 252 | 27.495 | 4.913 | 5.886 | 1.00 | 4.23 | | N |
| ATOM | 1448 | CA | LEU G | 252 | 28.283 | 6.032 | 6.369 | 1.00 | 4.22 | | C |
| ATOM | 1449 | CB | LEU G | 252 | 28.645 | 6.965 | 5.215 | 1.00 | 4.14 | | C |
| ATOM | 1450 | CG | LEU G | 252 | 30.017 | 6.731 | 4.581 | 1.00 | 4.05 | | C |
| ATOM | 1451 | CD1 | LEU G | 252 | 30.513 | 7.997 | 3.891 | 1.00 | 5.22 | | C |
| ATOM | 1452 | CD2 | LEU G | 252 | 31.023 | 6.242 | 5.617 | 1.00 | 6.20 | | C |
| ATOM | 1453 | C | LEU G | 252 | 27.515 | 6.799 | 7.432 | 1.00 | 4.62 | | C |
| ATOM | 1454 | O | LEU G | 252 | 26.389 | 7.238 | 7.203 | 1.00 | 4.97 | | O |
| ATOM | 1455 | N | THR G | 253 | 28.127 | 6.949 | 8.600 | 1.00 | 4.90 | | N |
| ATOM | 1456 | CA | THR G | 253 | 27.659 | 7.913 | 9.583 | 1.00 | 5.57 | | C |
| ATOM | 1457 | CB | THR G | 253 | 28.420 | 7.769 | 10.903 | 1.00 | 5.55 | | C |
| ATOM | 1458 | OG1 | THR G | 253 | 28.440 | 6.390 | 11.293 | 1.00 | 6.43 | | O |
| ATOM | 1459 | CG2 | THR G | 253 | 27.757 | 8.598 | 11.988 | 1.00 | 6.28 | | C |
| ATOM | 1460 | C | THR G | 253 | 27.865 | 9.329 | 9.069 | 1.00 | 6.03 | | C |
| ATOM | 1461 | O | THR G | 253 | 28.888 | 9.629 | 8.453 | 1.00 | 6.68 | | O |
| ATOM | 1462 | N | ASN G | 254 | 26.974 | 10.230 | 9.460 | 1.00 | 6.39 | | N |
| ATOM | 1463 | CA | ASN G | 254 | 27.257 | 11.649 | 9.342 | 1.00 | 6.79 | | C |
| ATOM | 1464 | CB | ASN G | 254 | 26.292 | 12.459 | 10.198 | 1.00 | 6.85 | | C |
| ATOM | 1465 | CG | ASN G | 254 | 26.317 | 13.927 | 9.853 | 1.00 | 6.81 | | C |
| ATOM | 1466 | OD1 | ASN G | 254 | 25.991 | 14.303 | 8.728 | 1.00 | 7.67 | | O |
| ATOM | 1467 | ND2 | ASN G | 254 | 26.919 | 14.726 | 10.726 | 1.00 | 6.19 | | N |
| ATOM | 1468 | C | ASN G | 254 | 28.685 | 11.958 | 9.760 | 1.00 | 7.08 | | C |
| ATOM | 1469 | O | ASN G | 254 | 29.495 | 12.426 | 8.961 | 1.00 | 6.90 | | O |
| ATOM | 1470 | N | SER G | 255 | 28.974 | 11.718 | 11.034 | 1.00 | 7.21 | | N |
| ATOM | 1471 | CA | SER G | 255 | 30.320 | 11.869 | 11.561 | 1.00 | 7.37 | | C |
| ATOM | 1472 | CB | SER G | 255 | 30.477 | 11.067 | 12.859 | 1.00 | 8.09 | | C |
| ATOM | 1473 | OG | SER G | 255 | 29.239 | 10.928 | 13.536 | 1.00 | 10.47 | | O |
| ATOM | 1474 | C | SER G | 255 | 31.365 | 11.429 | 10.538 | 1.00 | 6.82 | | C |
| ATOM | 1475 | O | SER G | 255 | 32.237 | 12.207 | 10.150 | 1.00 | 6.99 | | O |
| ATOM | 1476 | N | GLU G | 256 | 31.240 | 10.192 | 10.068 | 1.00 | 6.17 | | N |
| ATOM | 1477 | CA | GLU G | 256 | 32.218 | 9.629 | 9.152 | 1.00 | 6.13 | | C |
| ATOM | 1478 | CB | GLU G | 256 | 31.913 | 8.158 | 8.883 | 1.00 | 6.22 | | C |
| ATOM | 1479 | CG | GLU G | 256 | 32.296 | 7.240 | 10.026 | 1.00 | 8.41 | | C |
| ATOM | 1480 | CD | GLU G | 256 | 31.422 | 6.006 | 10.099 | 1.00 | 10.26 | | C |
| ATOM | 1481 | OE1 | GLU G | 256 | 30.637 | 5.776 | 9.156 | 1.00 | 11.90 | | O |
| ATOM | 1482 | OE2 | GLU G | 256 | 31.532 | 5.255 | 11.090 | 1.00 | 9.55 | | O |
| ATOM | 1483 | C | GLU G | 256 | 32.231 | 10.401 | 7.845 | 1.00 | 5.76 | | C |
| ATOM | 1484 | O | GLU G | 256 | 33.275 | 10.892 | 7.418 | 1.00 | 5.93 | | O |
| ATOM | 1485 | N | LEU G | 257 | 31.075 | 10.476 | 7.194 | 1.00 | 5.67 | | N |
| ATOM | 1486 | CA | LEU G | 257 | 30.972 | 11.191 | 5.930 | 1.00 | 5.37 | | C |
| ATOM | 1487 | CB | LEU G | 257 | 29.512 | 11.334 | 5.486 | 1.00 | 5.03 | | C |
| ATOM | 1488 | CG | LEU G | 257 | 29.282 | 12.309 | 4.325 | 1.00 | 3.65 | | C |
| ATOM | 1489 | CD1 | LEU G | 257 | 29.846 | 11.755 | 3.024 | 1.00 | 2.00 | | C |
| ATOM | 1490 | CD2 | LEU G | 257 | 27.810 | 12.657 | 4.172 | 1.00 | 2.14 | | C |
| ATOM | 1491 | C | LEU G | 257 | 31.623 | 12.563 | 6.048 | 1.00 | 5.64 | | C |
| ATOM | 1492 | O | LEU G | 257 | 32.401 | 12.970 | 5.185 | 1.00 | 5.81 | | O |
| ATOM | 1493 | N | LEU G | 258 | 31.353 | 13.249 | 7.153 | 1.00 | 5.75 | | N |
| ATOM | 1494 | CA | LEU G | 258 | 31.792 | 14.629 | 7.301 | 1.00 | 5.91 | | C |
| ATOM | 1495 | CB | LEU G | 258 | 31.162 | 15.276 | 8.535 | 1.00 | 6.10 | | C |
| ATOM | 1496 | CG | LEU G | 258 | 29.897 | 16.091 | 8.256 | 1.00 | 5.75 | | C |
| ATOM | 1497 | CD1 | LEU G | 258 | 29.230 | 16.525 | 9.552 | 1.00 | 6.33 | | C |
| ATOM | 1498 | CD2 | LEU G | 258 | 30.219 | 17.294 | 7.385 | 1.00 | 7.33 | | C |
| ATOM | 1499 | C | LEU G | 258 | 33.315 | 14.758 | 7.331 | 1.00 | 6.03 | | C |
| ATOM | 1500 | O | LEU G | 258 | 33.874 | 15.664 | 6.713 | 1.00 | 5.73 | | O |
| ATOM | 1501 | N | SER G | 259 | 33.983 | 13.867 | 8.061 | 1.00 | 6.47 | | N |
| ATOM | 1502 | CA | SER G | 259 | 35.442 | 13.887 | 8.139 | 1.00 | 6.95 | | C |
| ATOM | 1503 | CB | SER G | 259 | 35.929 | 13.137 | 9.381 | 1.00 | 7.05 | | C |
| ATOM | 1504 | OG | SER G | 259 | 36.284 | 11.802 | 9.064 | 1.00 | 6.86 | | O |
| ATOM | 1505 | C | SER G | 259 | 36.060 | 13.284 | 6.883 | 1.00 | 6.74 | | C |
| ATOM | 1506 | O | SER G | 259 | 37.173 | 13.638 | 6.493 | 1.00 | 6.86 | | O |
| ATOM | 1507 | N | LEU G | 260 | 35.311 | 12.400 | 6.234 | 1.00 | 6.31 | | N |
| ATOM | 1508 | CA | LEU G | 260 | 35.715 | 11.854 | 4.946 | 1.00 | 6.09 | | C |
| ATOM | 1509 | CB | LEU G | 260 | 34.747 | 10.756 | 4.507 | 1.00 | 6.09 | | C |
| ATOM | 1510 | CG | LEU G | 260 | 35.306 | 9.334 | 4.473 | 1.00 | 6.24 | | C |
| ATOM | 1511 | CD1 | LEU G | 260 | 34.338 | 8.388 | 3.779 | 1.00 | 6.53 | | C |
| ATOM | 1512 | CD2 | LEU G | 260 | 36.663 | 9.309 | 3.790 | 1.00 | 5.98 | | C |
| ATOM | 1513 | C | LEU G | 260 | 35.775 | 12.947 | 3.887 | 1.00 | 6.25 | | C |
| ATOM | 1514 | O | LEU G | 260 | 36.696 | 12.982 | 3.072 | 1.00 | 6.37 | | O |
| ATOM | 1515 | N | ILE G | 261 | 34.724 | 13.760 | 3.826 | 1.00 | 6.63 | | N |
| ATOM | 1516 | CA | ILE G | 261 | 34.729 | 14.952 | 2.987 | 1.00 | 7.19 | | C |
| ATOM | 1517 | CB | ILE G | 261 | 33.504 | 15.840 | 3.262 | 1.00 | 7.09 | | C |
| ATOM | 1518 | CG1 | ILE G | 261 | 32.292 | 15.350 | 2.474 | 1.00 | 7.46 | | C |
| ATOM | 1519 | CD1 | ILE G | 261 | 30.991 | 15.952 | 2.951 | 1.00 | 8.38 | | C |
| ATOM | 1520 | CG2 | ILE G | 261 | 33.809 | 17.290 | 2.916 | 1.00 | 6.61 | | C |
| ATOM | 1521 | C | ILE G | 261 | 35.966 | 15.768 | 3.307 | 1.00 | 8.01 | | C |
| ATOM | 1522 | O | ILE G | 261 | 36.716 | 16.162 | 2.414 | 1.00 | 8.26 | | O |
| ATOM | 1523 | N | ASN G | 262 | 36.157 | 16.040 | 4.592 | 1.00 | 9.05 | | N |
| ATOM | 1524 | CA | ASN G | 262 | 37.337 | 16.748 | 5.049 | 1.00 | 9.88 | | C |
| ATOM | 1525 | CB | ASN G | 262 | 37.517 | 16.575 | 6.555 | 1.00 | 9.99 | | C |
| ATOM | 1526 | CG | ASN G | 262 | 38.185 | 17.770 | 7.197 | 1.00 | 11.16 | | C |
| ATOM | 1527 | OD1 | ASN G | 262 | 38.493 | 18.756 | 6.526 | 1.00 | 12.70 | | O |
| ATOM | 1528 | ND2 | ASN G | 262 | 38.391 | 17.701 | 8.506 | 1.00 | 13.20 | | N |
| ATOM | 1529 | C | ASN G | 262 | 38.585 | 16.282 | 4.316 | 1.00 | 10.17 | | C |
| ATOM | 1530 | O | ASN G | 262 | 39.426 | 17.093 | 3.928 | 1.00 | 10.52 | | O |
| ATOM | 1531 | N | ASP G | 263 | 38.684 | 14.976 | 4.094 | 1.00 | 10.62 | | N |
| ATOM | 1532 | CA | ASP G | 263 | 39.925 | 14.380 | 3.621 | 1.00 | 11.02 | | C |
| ATOM | 1533 | CB | ASP G | 263 | 40.022 | 12.917 | 4.055 | 1.00 | 11.49 | | C |
| ATOM | 1534 | CG | ASP G | 263 | 41.297 | 12.251 | 3.577 | 1.00 | 12.80 | | C |
| ATOM | 1535 | OD1 | ASP G | 263 | 42.377 | 12.863 | 3.715 | 1.00 | 14.72 | | O |
| ATOM | 1536 | OD2 | ASP G | 263 | 41.220 | 11.113 | 3.068 | 1.00 | 13.88 | | O |
| ATOM | 1537 | C | ASP G | 263 | 40.067 | 14.491 | 2.107 | 1.00 | 10.92 | | C |
| ATOM | 1538 | O | ASP G | 263 | 41.135 | 14.224 | 1.556 | 1.00 | 10.96 | | O |
| ATOM | 1539 | N | MET G | 264 | 39.003 | 14.931 | 1.442 | 1.00 | 10.91 | | N |
| ATOM | 1540 | CA | MET G | 264 | 38.989 | 14.992 | −0.015 | 1.00 | 10.78 | | C |
| ATOM | 1541 | CB | MET G | 264 | 37.557 | 15.121 | −0.534 | 1.00 | 10.51 | | C |
| ATOM | 1542 | CG | MET G | 264 | 36.669 | 13.937 | −0.194 | 1.00 | 11.26 | | C |
| ATOM | 1543 | SD | MET G | 264 | 35.025 | 14.078 | −0.916 | 1.00 | 12.70 | | S |
| ATOM | 1544 | CE | MET G | 264 | 34.634 | 15.778 | −0.513 | 1.00 | 13.92 | | C |
| ATOM | 1545 | C | MET G | 264 | 39.851 | 16.135 | −0.543 | 1.00 | 10.85 | | C |
| ATOM | 1546 | O | MET G | 264 | 39.830 | 17.238 | 0.002 | 1.00 | 11.03 | | O |
| ATOM | 1547 | N | PRO G | 265 | 40.626 | 15.863 | −1.604 | 1.00 | 10.88 | | N |
| ATOM | 1548 | CA | PRO G | 265 | 41.558 | 16.827 | −2.184 | 1.00 | 11.09 | | C |
| ATOM | 1549 | CB | PRO G | 265 | 42.357 | 15.986 | −3.188 | 1.00 | 11.28 | | C |
| ATOM | 1550 | CG | PRO G | 265 | 41.513 | 14.787 | −3.453 | 1.00 | 11.24 | | C |
| ATOM | 1551 | CD | PRO G | 265 | 40.807 | 14.515 | −2.167 | 1.00 | 11.03 | | C |
| ATOM | 1552 | C | PRO G | 265 | 40.845 | 17.965 | −2.908 | 1.00 | 11.19 | | C |
| ATOM | 1553 | O | PRO G | 265 | 41.070 | 18.175 | −4.100 | 1.00 | 11.47 | | O |
| ATOM | 1554 | N | ILE G | 266 | 40.053 | 18.739 | −2.173 | 1.00 | 11.54 | | N |
| ATOM | 1555 | CA | ILE G | 266 | 39.282 | 19.820 | −2.777 | 1.00 | 12.15 | | C |
| ATOM | 1556 | CB | ILE G | 266 | 37.792 | 19.454 | −2.914 | 1.00 | 12.22 | | C |
| ATOM | 1557 | CG1 | ILE G | 266 | 37.245 | 18.928 | −1.585 | 1.00 | 12.53 | | C |
| ATOM | 1558 | CD1 | ILE G | 266 | 35.733 | 18.918 | −1.510 | 1.00 | 12.40 | | C |
| ATOM | 1559 | CG2 | ILE G | 266 | 37.594 | 18.431 | −4.023 | 1.00 | 12.74 | | C |
| ATOM | 1560 | C | ILE G | 266 | 39.413 | 21.132 | −2.013 | 1.00 | 12.29 | | C |
| ATOM | 1561 | O | ILE G | 266 | 39.853 | 21.156 | −0.863 | 1.00 | 12.36 | | O |
| ATOM | 1562 | N | THR G | 267 | 39.034 | 22.223 | −2.670 | 1.00 | 12.29 | | N |
| ATOM | 1563 | CA | THR G | 267 | 39.039 | 23.541 | −2.050 | 1.00 | 12.43 | | C |
| ATOM | 1564 | CB | THR G | 267 | 38.564 | 24.627 | −3.034 | 1.00 | 12.40 | | C |
| ATOM | 1565 | OG1 | THR G | 267 | 37.340 | 25.204 | −2.562 | 1.00 | 13.21 | | O |
| ATOM | 1566 | CG2 | THR G | 267 | 38.341 | 24.033 | −4.417 | 1.00 | 12.21 | | C |
| ATOM | 1567 | C | THR G | 267 | 38.141 | 23.560 | −0.819 | 1.00 | 12.37 | | C |
| ATOM | 1568 | O | THR G | 267 | 37.440 | 22.589 | −0.537 | 1.00 | 12.29 | | O |
| ATOM | 1569 | N | ASN G | 268 | 38.174 | 24.664 | −0.081 | 1.00 | 12.47 | | N |
| ATOM | 1570 | CA | ASN G | 268 | 37.523 | 24.720 | 1.219 | 1.00 | 12.57 | | C |
| ATOM | 1571 | CB | ASN G | 268 | 38.199 | 25.751 | 2.121 | 1.00 | 12.69 | | C |
| ATOM | 1572 | CG | ASN G | 268 | 39.221 | 25.127 | 3.048 | 1.00 | 13.65 | | C |
| ATOM | 1573 | OD1 | ASN G | 268 | 39.463 | 23.921 | 3.000 | 1.00 | 14.37 | | O |
| ATOM | 1574 | ND2 | ASN G | 268 | 39.843 | 25.949 | 3.884 | 1.00 | 15.74 | | N |
| ATOM | 1575 | C | ASN G | 268 | 36.028 | 24.995 | 1.123 | 1.00 | 12.43 | | C |
| ATOM | 1576 | O | ASN G | 268 | 35.232 | 24.403 | 1.853 | 1.00 | 12.51 | | O |
| ATOM | 1577 | N | ASP G | 269 | 35.655 | 25.926 | 0.252 | 1.00 | 12.34 | | N |
| ATOM | 1578 | CA | ASP G | 269 | 34.248 | 26.218 | 0.008 | 1.00 | 12.33 | | C |
| ATOM | 1579 | CB | ASP G | 269 | 34.106 | 27.359 | −0.999 | 1.00 | 12.89 | | C |
| ATOM | 1580 | CG | ASP G | 269 | 34.761 | 28.639 | −0.525 | 1.00 | 14.84 | | C |
| ATOM | 1581 | OD1 | ASP G | 269 | 34.594 | 28.989 | 0.663 | 1.00 | 16.90 | | O |
| ATOM | 1582 | OD2 | ASP G | 269 | 35.473 | 29.277 | −1.328 | 1.00 | 17.65 | | O |
| ATOM | 1583 | C | ASP G | 269 | 33.532 | 24.978 | −0.506 | 1.00 | 11.60 | | C |
| ATOM | 1584 | O | ASP G | 269 | 32.411 | 24.679 | −0.095 | 1.00 | 11.65 | | O |
| ATOM | 1585 | N | GLN G | 270 | 34.189 | 24.260 | −1.410 | 1.00 | 10.62 | | N |
| ATOM | 1586 | CA | GLN G | 270 | 33.728 | 22.943 | −1.816 | 1.00 | 10.01 | | C |
| ATOM | 1587 | CB | GLN G | 270 | 34.795 | 22.236 | −2.647 | 1.00 | 10.18 | | C |
| ATOM | 1588 | CG | GLN G | 270 | 34.248 | 21.533 | −3.870 | 1.00 | 11.49 | | C |
| ATOM | 1589 | CD | GLN G | 270 | 35.200 | 21.598 | −5.043 | 1.00 | 14.38 | | C |
| ATOM | 1590 | OE1 | GLN G | 270 | 35.516 | 20.579 | −5.655 | 1.00 | 16.38 | | O |
| ATOM | 1591 | NE2 | GLN G | 270 | 35.718 | 22.789 | −5.318 | 1.00 | 15.41 | | N |
| ATOM | 1592 | C | GLN G | 270 | 33.366 | 22.094 | −0.606 | 1.00 | 9.48 | | C |
| ATOM | 1593 | O | GLN G | 270 | 32.213 | 21.698 | −0.437 | 1.00 | 9.12 | | O |
| ATOM | 1594 | N | LYS G | 271 | 34.358 | 21.809 | 0.231 | 1.00 | 9.19 | | N |
| ATOM | 1595 | CA | LYS G | 271 | 34.126 | 21.040 | 1.445 | 1.00 | 8.89 | | C |
| ATOM | 1596 | CB | LYS G | 271 | 35.362 | 21.070 | 2.345 | 1.00 | 8.52 | | C |
| ATOM | 1597 | CG | LYS G | 271 | 36.541 | 20.285 | 1.797 | 1.00 | 9.14 | | C |
| ATOM | 1598 | CD | LYS G | 271 | 37.643 | 20.147 | 2.832 | 1.00 | 10.72 | | C |
| ATOM | 1599 | CE | LYS G | 271 | 39.013 | 20.137 | 2.176 | 1.00 | 10.79 | | C |
| ATOM | 1600 | NZ | LYS G | 271 | 40.035 | 19.472 | 3.030 | 1.00 | 9.77 | | N |
| ATOM | 1601 | C | LYS G | 271 | 32.907 | 21.563 | 2.199 | 1.00 | 8.83 | | C |
| ATOM | 1602 | O | LYS G | 271 | 31.959 | 20.820 | 2.452 | 1.00 | 9.05 | | O |
| ATOM | 1603 | N | LYS G | 272 | 32.899 | 22.863 | 2.474 | 1.00 | 8.65 | | N |
| ATOM | 1604 | CA | LYS G | 272 | 31.789 | 23.482 | 3.187 | 1.00 | 8.50 | | C |
| ATOM | 1605 | CB | LYS G | 272 | 31.997 | 24.992 | 3.304 | 1.00 | 9.14 | | C |
| ATOM | 1606 | CG | LYS G | 272 | 30.762 | 25.748 | 3.766 | 1.00 | 11.58 | | C |
| ATOM | 1607 | CD | LYS G | 272 | 30.925 | 27.246 | 3.575 | 1.00 | 15.78 | | C |
| ATOM | 1608 | CE | LYS G | 272 | 29.995 | 28.021 | 4.492 | 1.00 | 17.65 | | C |
| ATOM | 1609 | NZ | LYS G | 272 | 30.462 | 29.418 | 4.703 | 1.00 | 19.68 | | N |
| ATOM | 1610 | C | LYS G | 272 | 30.458 | 23.191 | 2.503 | 1.00 | 7.88 | | C |
| ATOM | 1611 | O | LYS G | 272 | 29.486 | 22.808 | 3.154 | 1.00 | 8.12 | | O |
| ATOM | 1612 | N | LEU G | 273 | 30.408 | 23.416 | 1.194 | 1.00 | 6.95 | | N |
| ATOM | 1613 | CA | LEU G | 273 | 29.217 | 23.104 | 0.413 | 1.00 | 5.70 | | C |
| ATOM | 1614 | CB | LEU G | 273 | 29.469 | 23.344 | −1.077 | 1.00 | 5.85 | | C |
| ATOM | 1615 | CG | LEU G | 273 | 28.313 | 23.000 | −2.020 | 1.00 | 4.65 | | C |
| ATOM | 1616 | CD1 | LEU G | 273 | 27.277 | 24.114 | −2.032 | 1.00 | 4.57 | | C |
| ATOM | 1617 | CD2 | LEU G | 273 | 28.829 | 22.729 | −3.425 | 1.00 | 3.69 | | C |
| ATOM | 1618 | C | LEU G | 273 | 28.781 | 21.663 | 0.644 | 1.00 | 5.14 | | C |
| ATOM | 1619 | O | LEU G | 273 | 27.678 | 21.409 | 1.128 | 1.00 | 5.25 | | O |
| ATOM | 1620 | N | MET G | 274 | 29.665 | 20.724 | 0.327 | 1.00 | 4.52 | | N |
| ATOM | 1621 | CA | MET G | 274 | 29.354 | 19.312 | 0.482 | 1.00 | 4.59 | | C |
| ATOM | 1622 | CB | MET G | 274 | 30.573 | 18.454 | 0.152 | 1.00 | 4.35 | | C |
| ATOM | 1623 | CG | MET G | 274 | 30.938 | 18.457 | −1.319 | 1.00 | 4.50 | | C |
| ATOM | 1624 | SD | MET G | 274 | 32.305 | 17.344 | −1.679 | 1.00 | 5.51 | | S |
| ATOM | 1625 | CE | MET G | 274 | 31.469 | 15.758 | −1.633 | 1.00 | 5.26 | | C |
| ATOM | 1626 | C | MET G | 274 | 28.864 | 19.014 | 1.893 | 1.00 | 5.18 | | C |
| ATOM | 1627 | O | MET G | 274 | 27.893 | 18.280 | 2.075 | 1.00 | 5.61 | | O |
| ATOM | 1628 | N | SER G | 275 | 29.457 | 19.689 | 2.875 | 1.00 | 5.89 | | N |
| ATOM | 1629 | CA | SER G | 275 | 29.118 | 19.465 | 4.278 | 1.00 | 6.18 | | C |
| ATOM | 1630 | CB | SER G | 275 | 30.161 | 20.108 | 5.194 | 1.00 | 6.33 | | C |
| ATOM | 1631 | OG | SER G | 275 | 31.457 | 19.601 | 4.925 | 1.00 | 5.91 | | O |
| ATOM | 1632 | C | SER G | 275 | 27.726 | 19.989 | 4.619 | 1.00 | 6.58 | | C |
| ATOM | 1633 | O | SER G | 275 | 27.031 | 19.425 | 5.464 | 1.00 | 7.03 | | O |
| ATOM | 1634 | N | ASN G | 276 | 27.313 | 21.054 | 3.940 | 1.00 | 7.07 | | N |
| ATOM | 1635 | CA | ASN G | 276 | 25.979 | 21.606 | 4.140 | 1.00 | 7.86 | | C |
| ATOM | 1636 | CB | ASN G | 276 | 25.987 | 23.126 | 3.942 | 1.00 | 8.34 | | C |
| ATOM | 1637 | CG | ASN G | 276 | 26.343 | 23.881 | 5.214 | 1.00 | 9.77 | | C |
| ATOM | 1638 | OD1 | ASN G | 276 | 26.033 | 23.439 | 6.320 | 1.00 | 11.84 | | O |
| ATOM | 1639 | ND2 | ASN G | 276 | 26.964 | 25.044 | 5.057 | 1.00 | 11.15 | | N |
| ATOM | 1640 | C | ASN G | 276 | 24.908 | 20.954 | 3.262 | 1.00 | 7.71 | | C |
| ATOM | 1641 | O | ASN G | 276 | 23.780 | 21.442 | 3.191 | 1.00 | 7.84 | | O |
| ATOM | 1642 | N | ASN G | 277 | 25.239 | 19.819 | 2.649 | 1.00 | 7.77 | | N |
| ATOM | 1643 | CA | ASN G | 277 | 24.372 | 19.220 | 1.631 | 1.00 | 7.85 | | C |
| ATOM | 1644 | CB | ASN G | 277 | 24.704 | 19.777 | 0.246 | 1.00 | 7.58 | | C |
| ATOM | 1645 | CG | ASN G | 277 | 24.023 | 21.099 | −0.027 | 1.00 | 8.29 | | C |
| ATOM | 1646 | OD1 | ASN G | 277 | 22.818 | 21.152 | −0.272 | 1.00 | 9.04 | | O |
| ATOM | 1647 | ND2 | ASN G | 277 | 24.798 | 22.176 | −0.008 | 1.00 | 9.57 | | N |
| ATOM | 1648 | C | ASN G | 277 | 24.410 | 17.693 | 1.590 | 1.00 | 7.86 | | C |
| ATOM | 1649 | O | ASN G | 277 | 24.154 | 17.089 | 0.549 | 1.00 | 7.85 | | O |
| ATOM | 1650 | N | VAL G | 278 | 24.886 | 17.093 | 2.674 | 1.00 | 7.67 | | N |
| ATOM | 1651 | CA | VAL G | 278 | 24.839 | 15.645 | 2.879 | 1.00 | 7.77 | | C |
| ATOM | 1652 | CB | VAL G | 278 | 24.594 | 15.309 | 4.352 | 1.00 | 7.98 | | C |
| ATOM | 1653 | CG1 | VAL G | 278 | 25.915 | 15.261 | 5.100 | 1.00 | 7.21 | | C |
| ATOM | 1654 | CG2 | VAL G | 278 | 23.655 | 16.332 | 4.977 | 1.00 | 8.99 | | C |
| ATOM | 1655 | C | VAL G | 278 | 23.843 | 14.865 | 2.019 | 1.00 | 7.76 | | C |
| ATOM | 1656 | O | VAL G | 278 | 24.183 | 13.811 | 1.483 | 1.00 | 7.84 | | O |
| ATOM | 1657 | N | GLN G | 279 | 22.569 | 15.228 | 2.118 | 1.00 | 7.47 | | N |
| ATOM | 1658 | CA | GLN G | 279 | 21.510 | 14.444 | 1.492 | 1.00 | 7.07 | | C |
| ATOM | 1659 | CB | GLN G | 279 | 20.187 | 15.204 | 1.550 | 1.00 | 7.62 | | C |
| ATOM | 1660 | CG | GLN G | 279 | 19.021 | 14.376 | 2.044 | 1.00 | 10.35 | | C |
| ATOM | 1661 | CD | GLN G | 279 | 17.682 | 14.997 | 1.702 | 1.00 | 13.70 | | C |
| ATOM | 1662 | OE1 | GLN G | 279 | 17.612 | 16.002 | 0.996 | 1.00 | 15.06 | | O |
| ATOM | 1663 | NE2 | GLN G | 279 | 16.607 | 14.383 | 2.179 | 1.00 | 13.97 | | N |
| ATOM | 1664 | C | GLN G | 279 | 21.852 | 14.121 | 0.042 | 1.00 | 6.02 | | C |
| ATOM | 1665 | O | GLN G | 279 | 22.032 | 12.958 | −0.323 | 1.00 | 5.45 | | O |
| ATOM | 1666 | N | ILE G | 280 | 21.863 | 15.154 | −0.792 | 1.00 | 5.25 | | N |
| ATOM | 1667 | CA | ILE G | 280 | 22.325 | 15.034 | −2.169 | 1.00 | 4.74 | | C |
| ATOM | 1668 | CB | ILE G | 280 | 22.650 | 16.408 | −2.771 | 1.00 | 4.58 | | C |
| ATOM | 1669 | CG1 | ILE G | 280 | 21.527 | 17.396 | −2.461 | 1.00 | 4.16 | | C |
| ATOM | 1670 | CD1 | ILE G | 280 | 20.235 | 17.078 | −3.177 | 1.00 | 3.88 | | C |
| ATOM | 1671 | CG2 | ILE G | 280 | 22.835 | 16.293 | −4.271 | 1.00 | 3.92 | | C |
| ATOM | 1672 | C | ILE G | 280 | 23.550 | 14.135 | −2.286 | 1.00 | 4.43 | | C |
| ATOM | 1673 | O | ILE G | 280 | 23.493 | 13.081 | −2.917 | 1.00 | 4.39 | | O |
| ATOM | 1674 | N | VAL G | 281 | 24.676 | 14.596 | −1.749 | 1.00 | 4.05 | | N |
| ATOM | 1675 | CA | VAL G | 281 | 25.906 | 13.816 | −1.795 | 1.00 | 3.58 | | C |
| ATOM | 1676 | CB | VAL G | 281 | 26.918 | 14.251 | −0.714 | 1.00 | 3.13 | | C |
| ATOM | 1677 | CG1 | VAL G | 281 | 28.296 | 13.684 | −1.026 | 1.00 | 2.34 | | C |
| ATOM | 1678 | CG2 | VAL G | 281 | 26.978 | 15.768 | −0.616 | 1.00 | 2.21 | | C |
| ATOM | 1679 | C | VAL G | 281 | 25.593 | 12.335 | −1.626 | 1.00 | 3.96 | | C |
| ATOM | 1680 | O | VAL G | 281 | 25.875 | 11.534 | −2.515 | 1.00 | 4.15 | | O |
| ATOM | 1681 | N | ARG G | 282 | 24.908 | 11.994 | −0.539 | 1.00 | 4.23 | | N |
| ATOM | 1682 | CA | ARG G | 282 | 24.500 | 10.615 | −0.296 | 1.00 | 4.45 | | C |
| ATOM | 1683 | CB | ARG G | 282 | 23.570 | 10.540 | 0.911 | 1.00 | 3.96 | | C |
| ATOM | 1684 | CG | ARG G | 282 | 24.225 | 10.953 | 2.203 | 1.00 | 2.99 | | C |
| ATOM | 1685 | CD | ARG G | 282 | 23.924 | 9.962 | 3.303 | 1.00 | 2.28 | | C |
| ATOM | 1686 | NE | ARG G | 282 | 24.332 | 10.474 | 4.605 | 1.00 | 2.00 | | N |
| ATOM | 1687 | CZ | ARG G | 282 | 25.026 | 9.777 | 5.498 | 1.00 | 2.44 | | C |
| ATOM | 1688 | NH1 | ARG G | 282 | 25.359 | 8.517 | 5.251 | 1.00 | 2.04 | | N |
| ATOM | 1689 | NH2 | ARG G | 282 | 25.369 | 10.333 | 6.650 | 1.00 | 3.44 | | N |
| ATOM | 1690 | C | ARG G | 282 | 23.798 | 10.039 | −1.516 | 1.00 | 5.32 | | C |
| ATOM | 1691 | O | ARG G | 282 | 24.143 | 8.958 | −1.997 | 1.00 | 5.95 | | O |
| ATOM | 1692 | N | GLN G | 283 | 22.800 | 10.765 | −2.006 | 1.00 | 5.50 | | N |
| ATOM | 1693 | CA | GLN G | 283 | 22.045 | 10.327 | −3.169 | 1.00 | 5.80 | | C |
| ATOM | 1694 | CB | GLN G | 283 | 20.910 | 11.303 | −3.471 | 1.00 | 6.19 | | C |
| ATOM | 1695 | CG | GLN G | 283 | 19.768 | 11.219 | −2.480 | 1.00 | 9.68 | | C |
| ATOM | 1696 | CD | GLN G | 283 | 18.840 | 12.406 | −2.561 | 1.00 | 13.92 | | C |
| ATOM | 1697 | OE1 | GLN G | 283 | 18.991 | 13.269 | −3.426 | 1.00 | 16.20 | | O |
| ATOM | 1698 | NE2 | GLN G | 283 | 17.833 | 12.428 | −1.696 | 1.00 | 14.17 | | N |
| ATOM | 1699 | C | GLN G | 283 | 22.953 | 10.175 | −4.379 | 1.00 | 5.44 | | C |
| ATOM | 1700 | O | GLN G | 283 | 22.687 | 9.364 | −5.264 | 1.00 | 5.43 | | O |
| ATOM | 1701 | N | GLN G | 284 | 24.067 | 10.896 | −4.371 | 1.00 | 5.26 | | N |
| ATOM | 1702 | CA | GLN G | 284 | 24.957 | 10.920 | −5.521 | 1.00 | 5.18 | | C |
| ATOM | 1703 | CB | GLN G | 284 | 25.568 | 12.307 | −5.702 | 1.00 | 5.32 | | C |
| ATOM | 1704 | CG | GLN G | 284 | 24.813 | 13.189 | −6.673 | 1.00 | 6.85 | | C |
| ATOM | 1705 | CD | GLN G | 284 | 25.434 | 14.560 | −6.804 | 1.00 | 9.21 | | C |
| ATOM | 1706 | OE1 | GLN G | 284 | 25.863 | 15.160 | −5.819 | 1.00 | 9.74 | | O |
| ATOM | 1707 | NE2 | GLN G | 284 | 25.492 | 15.064 | −8.027 | 1.00 | 9.79 | | N |
| ATOM | 1708 | C | GLN G | 284 | 26.060 | 9.882 | −5.400 | 1.00 | 4.67 | | C |
| ATOM | 1709 | O | GLN G | 284 | 26.884 | 9.736 | −6.302 | 1.00 | 4.19 | | O |
| ATOM | 1710 | N | SER G | 285 | 26.109 | 9.199 | −4.262 | 1.00 | 4.51 | | N |
| ATOM | 1711 | CA | SER G | 285 | 27.204 | 8.281 | −3.990 | 1.00 | 4.39 | | C |
| ATOM | 1712 | CB | SER G | 285 | 27.767 | 8.510 | −2.587 | 1.00 | 4.13 | | C |
| ATOM | 1713 | OG | SER G | 285 | 28.180 | 9.856 | −2.421 | 1.00 | 4.40 | | O |
| ATOM | 1714 | C | SER G | 285 | 26.767 | 6.834 | −4.166 | 1.00 | 4.44 | | C |
| ATOM | 1715 | O | SER G | 285 | 25.587 | 6.555 | −4.382 | 1.00 | 4.82 | | O |
| ATOM | 1716 | N | TYR G | 286 | 27.751 | 5.949 | −4.271 | 1.00 | 4.53 | | N |
| ATOM | 1717 | CA | TYR G | 286 | 27.500 | 4.517 | −4.291 | 1.00 | 4.80 | | C |
| ATOM | 1718 | CB | TYR G | 286 | 27.978 | 3.915 | −5.612 | 1.00 | 4.88 | | C |
| ATOM | 1719 | CG | TYR G | 286 | 27.168 | 4.354 | −6.809 | 1.00 | 5.68 | | C |
| ATOM | 1720 | CD1 | TYR G | 286 | 27.156 | 5.681 | −7.218 | 1.00 | 7.34 | | C |
| ATOM | 1721 | CE1 | TYR G | 286 | 26.429 | 6.081 | −8.322 | 1.00 | 9.02 | | C |
| ATOM | 1722 | CZ | TYR G | 286 | 25.720 | 5.145 | −9.044 | 1.00 | 9.40 | | C |
| ATOM | 1723 | OH | TYR G | 286 | 24.998 | 5.538 | −10.146 | 1.00 | 10.44 | | O |
| ATOM | 1724 | CE2 | TYR G | 286 | 25.730 | 3.820 | −8.668 | 1.00 | 9.38 | | C |
| ATOM | 1725 | CD2 | TYR G | 286 | 26.444 | 3.433 | −7.553 | 1.00 | 7.39 | | C |
| ATOM | 1726 | C | TYR G | 286 | 28.208 | 3.845 | −3.124 | 1.00 | 4.76 | | C |
| ATOM | 1727 | O | TYR G | 286 | 29.130 | 4.411 | −2.541 | 1.00 | 5.25 | | O |
| ATOM | 1728 | N | SER G | 287 | 27.765 | 2.642 | −2.779 | 1.00 | 4.52 | | N |
| ATOM | 1729 | CA | SER G | 287 | 28.528 | 1.786 | −1.884 | 1.00 | 4.45 | | C |
| ATOM | 1730 | CB | SER G | 287 | 27.888 | 1.743 | −0.496 | 1.00 | 4.57 | | C |
| ATOM | 1731 | OG | SER G | 287 | 28.421 | 0.681 | 0.275 | 1.00 | 4.38 | | O |
| ATOM | 1732 | C | SER G | 287 | 28.670 | 0.381 | −2.454 | 1.00 | 4.61 | | C |
| ATOM | 1733 | O | SER G | 287 | 27.683 | −0.253 | −2.820 | 1.00 | 4.40 | | O |
| ATOM | 1734 | N | ILE G | 288 | 29.914 | −0.002 | −2.717 | 1.00 | 5.11 | | N |
| ATOM | 1735 | CA | ILE G | 288 | 30.227 | −1.318 | −3.258 | 1.00 | 5.81 | | C |
| ATOM | 1736 | CB | ILE G | 288 | 31.376 | −1.250 | −4.288 | 1.00 | 5.52 | | C |
| ATOM | 1737 | CG1 | ILE G | 288 | 31.377 | 0.095 | −5.021 | 1.00 | 5.47 | | C |
| ATOM | 1738 | CD1 | ILE G | 288 | 31.007 | 0.002 | −6.487 | 1.00 | 6.23 | | C |
| ATOM | 1739 | CG2 | ILE G | 288 | 31.298 | −2.421 | −5.256 | 1.00 | 6.22 | | C |
| ATOM | 1740 | C | ILE G | 288 | 30.664 | −2.245 | −2.138 | 1.00 | 6.71 | | C |
| ATOM | 1741 | O | ILE G | 288 | 31.615 | −1.948 | −1.414 | 1.00 | 7.30 | | O |
| ATOM | 1742 | N | MET G | 289 | 30.085 | −3.438 | −2.118 | 1.00 | 7.71 | | N |
| ATOM | 1743 | CA | MET G | 289 | 30.585 | −4.515 | −1.277 | 1.00 | 8.57 | | C |
| ATOM | 1744 | CB | MET G | 289 | 29.605 | −5.692 | −1.306 | 1.00 | 8.71 | | C |
| ATOM | 1745 | CG | MET G | 289 | 29.648 | −6.584 | −0.077 | 1.00 | 9.70 | | C |
| ATOM | 1746 | SD | MET G | 289 | 28.615 | −8.046 | −0.280 | 1.00 | 12.59 | | S |
| ATOM | 1747 | CE | MET G | 289 | 29.227 | −8.678 | −1.839 | 1.00 | 12.41 | | C |
| ATOM | 1748 | C | MET G | 289 | 31.975 | −4.963 | −1.736 | 1.00 | 9.12 | | C |
| ATOM | 1749 | O | MET G | 289 | 32.231 | −5.086 | −2.935 | 1.00 | 9.31 | | O |
| ATOM | 1750 | N | SER G | 290 | 32.891 | −5.122 | −0.784 | 1.00 | 9.80 | | N |
| ATOM | 1751 | CA | SER G | 290 | 34.272 | −5.469 | −1.098 | 1.00 | 10.49 | | C |
| ATOM | 1752 | CB | SER G | 290 | 35.242 | −4.513 | −0.401 | 1.00 | 10.64 | | C |
| ATOM | 1753 | OG | SER G | 290 | 36.498 | −4.486 | −1.058 | 1.00 | 11.99 | | O |
| ATOM | 1754 | C | SER G | 290 | 34.592 | −6.918 | −0.742 | 1.00 | 10.75 | | C |
| ATOM | 1755 | O | SER G | 290 | 34.631 | −7.778 | −1.621 | 1.00 | 10.89 | | O |
| ATOM | 1756 | N | ILE G | 291 | 34.760 | −7.206 | 0.544 | 1.00 | 11.04 | | N |
| ATOM | 1757 | CA | ILE G | 291 | 34.884 | −8.596 | 0.965 | 1.00 | 11.41 | | C |
| ATOM | 1758 | CB | ILE G | 291 | 36.329 | −9.120 | 0.900 | 1.00 | 11.80 | | C |
| ATOM | 1759 | CG1 | ILE G | 291 | 37.332 | −7.977 | 1.061 | 1.00 | 11.81 | | C |
| ATOM | 1760 | CD1 | ILE G | 291 | 38.227 | −8.113 | 2.277 | 1.00 | 11.76 | | C |
| ATOM | 1761 | CG2 | ILE G | 291 | 36.561 | −9.846 | −0.416 | 1.00 | 12.39 | | C |
| ATOM | 1762 | C | ILE G | 291 | 34.214 | −8.982 | 2.277 | 1.00 | 11.13 | | C |
| ATOM | 1763 | O | ILE G | 291 | 34.299 | −8.268 | 3.277 | 1.00 | 10.55 | | O |
| ATOM | 1764 | N | ILE G | 292 | 33.536 | −10.124 | 2.246 | 1.00 | 11.36 | | N |
| ATOM | 1765 | CA | ILE G | 292 | 32.802 | −10.620 | 3.398 | 1.00 | 11.82 | | C |
| ATOM | 1766 | CB | ILE G | 292 | 31.359 | −11.016 | 3.029 | 1.00 | 11.86 | | C |
| ATOM | 1767 | CG1 | ILE G | 292 | 31.087 | −10.734 | 1.550 | 1.00 | 13.42 | | C |
| ATOM | 1768 | CD1 | ILE G | 292 | 31.289 | −9.282 | 1.158 | 1.00 | 15.86 | | C |
| ATOM | 1769 | CG2 | ILE G | 292 | 30.358 | −10.287 | 3.923 | 1.00 | 10.85 | | C |
| ATOM | 1770 | C | ILE G | 292 | 33.505 | −11.826 | 3.992 | 1.00 | 11.72 | | C |
| ATOM | 1771 | O | ILE G | 292 | 33.683 | −12.849 | 3.330 | 1.00 | 11.28 | | O |
| ATOM | 1772 | N | LYS G | 293 | 33.946 | −11.679 | 5.234 | 1.00 | 12.11 | | N |
| ATOM | 1773 | CA | LYS G | 293 | 34.275 | −12.823 | 6.058 | 1.00 | 12.92 | | C |
| ATOM | 1774 | CB | LYS G | 293 | 35.715 | −12.720 | 6.553 | 1.00 | 13.15 | | C |
| ATOM | 1775 | CG | LYS G | 293 | 36.674 | −12.179 | 5.512 | 1.00 | 14.17 | | C |
| ATOM | 1776 | CD | LYS G | 293 | 38.000 | −12.910 | 5.561 | 1.00 | 15.37 | | C |
| ATOM | 1777 | CE | LYS G | 293 | 38.720 | −12.826 | 4.229 | 1.00 | 16.68 | | C |
| ATOM | 1778 | NZ | LYS G | 293 | 39.984 | −13.607 | 4.242 | 1.00 | 18.18 | | N |
| ATOM | 1779 | C | LYS G | 293 | 33.310 | −12.923 | 7.232 | 1.00 | 13.32 | | C |
| ATOM | 1780 | O | LYS G | 293 | 32.633 | −11.952 | 7.578 | 1.00 | 13.17 | | O |
| ATOM | 1781 | N | GLU G | 294 | 33.140 | −14.141 | 7.735 | 1.00 | 14.07 | | N |
| ATOM | 1782 | CA | GLU G | 294 | 32.217 | −14.398 | 8.831 | 1.00 | 15.07 | | C |
| ATOM | 1783 | CB | GLU G | 294 | 32.474 | −15.786 | 9.425 | 1.00 | 15.94 | | C |
| ATOM | 1784 | CG | GLU G | 294 | 32.183 | −16.935 | 8.471 | 1.00 | 19.87 | | C |
| ATOM | 1785 | CD | GLU G | 294 | 32.165 | −18.282 | 9.169 | 1.00 | 23.88 | | C |
| ATOM | 1786 | OE1 | GLU G | 294 | 31.348 | −18.465 | 10.097 | 1.00 | 23.75 | | O |
| ATOM | 1787 | OE2 | GLU G | 294 | 32.950 | −19.168 | 8.766 | 1.00 | 25.81 | | O |
| ATOM | 1788 | C | GLU G | 294 | 32.301 | −13.320 | 9.917 | 1.00 | 14.40 | | C |
| ATOM | 1789 | O | GLU G | 294 | 31.281 | −12.936 | 10.491 | 1.00 | 14.33 | | O |
| ATOM | 1790 | N | GLU G | 295 | 33.502 | −12.779 | 10.132 | 1.00 | 13.97 | | N |
| ATOM | 1791 | CA | GLU G | 295 | 33.765 | −11.861 | 11.251 | 1.00 | 13.84 | | C |
| ATOM | 1792 | CB | GLU G | 295 | 34.681 | −12.508 | 12.299 | 1.00 | 14.67 | | C |
| ATOM | 1793 | CG | GLU G | 295 | 35.496 | −13.688 | 11.794 | 1.00 | 17.56 | | C |
| ATOM | 1794 | CD | GLU G | 295 | 36.393 | −13.328 | 10.626 | 1.00 | 21.41 | | C |
| ATOM | 1795 | OE1 | GLU G | 295 | 36.085 | −13.752 | 9.493 | 1.00 | 22.35 | | O |
| ATOM | 1796 | OE2 | GLU G | 295 | 37.472 | −12.745 | 10.861 | 1.00 | 23.20 | | O |
| ATOM | 1797 | C | GLU G | 295 | 34.340 | −10.502 | 10.827 | 1.00 | 12.77 | | C |
| ATOM | 1798 | O | GLU G | 295 | 34.771 | −9.712 | 11.670 | 1.00 | 12.58 | | O |
| ATOM | 1799 | N | VAL G | 296 | 34.389 | −10.256 | 9.522 | 1.00 | 11.72 | | N |
| ATOM | 1800 | CA | VAL G | 296 | 34.634 | −8.911 | 9.011 | 1.00 | 10.65 | | C |
| ATOM | 1801 | CB | VAL G | 296 | 36.143 | −8.604 | 8.894 | 1.00 | 10.47 | | C |
| ATOM | 1802 | CG1 | VAL G | 296 | 36.369 | −7.112 | 8.687 | 1.00 | 9.77 | | C |
| ATOM | 1803 | CG2 | VAL G | 296 | 36.879 | −9.090 | 10.133 | 1.00 | 11.31 | | C |
| ATOM | 1804 | C | VAL G | 296 | 33.949 | −8.664 | 7.669 | 1.00 | 10.26 | | C |
| ATOM | 1805 | O | VAL G | 296 | 33.931 | −9.530 | 6.793 | 1.00 | 10.09 | | O |
| ATOM | 1806 | N | LEU G | 297 | 33.379 | −7.473 | 7.525 | 1.00 | 9.77 | | N |
| ATOM | 1807 | CA | LEU G | 297 | 32.842 | −7.027 | 6.250 | 1.00 | 8.97 | | C |
| ATOM | 1808 | CB | LEU G | 297 | 31.332 | −6.796 | 6.367 | 1.00 | 8.64 | | C |
| ATOM | 1809 | CG | LEU G | 297 | 30.643 | −6.020 | 5.243 | 1.00 | 9.01 | | C |
| ATOM | 1810 | CD1 | LEU G | 297 | 30.733 | −6.773 | 3.927 | 1.00 | 9.79 | | C |
| ATOM | 1811 | CD2 | LEU G | 297 | 29.194 | −5.752 | 5.606 | 1.00 | 9.43 | | C |
| ATOM | 1812 | C | LEU G | 297 | 33.544 | −5.745 | 5.821 | 1.00 | 8.34 | | C |
| ATOM | 1813 | O | LEU G | 297 | 33.645 | −4.793 | 6.595 | 1.00 | 8.64 | | O |
| ATOM | 1814 | N | ALA G | 298 | 34.131 | −5.767 | 4.631 | 1.00 | 7.21 | | N |
| ATOM | 1815 | CA | ALA G | 298 | 34.751 | −4.573 | 4.079 | 1.00 | 6.33 | | C |
| ATOM | 1816 | CB | ALA G | 298 | 36.231 | −4.800 | 3.860 | 1.00 | 6.68 | | C |
| ATOM | 1817 | C | ALA G | 298 | 34.074 | −4.177 | 2.778 | 1.00 | 5.85 | | C |
| ATOM | 1818 | O | ALA G | 298 | 33.727 | −5.033 | 1.965 | 1.00 | 5.96 | | O |
| ATOM | 1819 | N | TYR G | 299 | 33.820 | −2.883 | 2.620 | 1.00 | 5.28 | | N |
| ATOM | 1820 | CA | TYR G | 299 | 33.178 | −2.376 | 1.417 | 1.00 | 4.63 | | C |
| ATOM | 1821 | CB | TYR G | 299 | 31.660 | −2.340 | 1.595 | 1.00 | 4.36 | | C |
| ATOM | 1822 | CG | TYR G | 299 | 31.204 | −1.517 | 2.776 | 1.00 | 4.34 | | C |
| ATOM | 1823 | CD1 | TYR G | 299 | 30.967 | −0.156 | 2.648 | 1.00 | 4.48 | | C |
| ATOM | 1824 | CE1 | TYR G | 299 | 30.505 | 0.592 | 3.717 | 1.00 | 4.55 | | C |
| ATOM | 1825 | CZ | TYR G | 299 | 30.292 | −0.019 | 4.937 | 1.00 | 3.72 | | C |
| ATOM | 1826 | OH | TYR G | 299 | 29.847 | 0.720 | 6.009 | 1.00 | 2.38 | | O |
| ATOM | 1827 | CE2 | TYR G | 299 | 30.540 | −1.366 | 5.092 | 1.00 | 4.13 | | C |
| ATOM | 1828 | CD2 | TYR G | 299 | 31.007 | −2.102 | 4.020 | 1.00 | 4.38 | | C |
| ATOM | 1829 | C | TYR G | 299 | 33.692 | −0.988 | 1.063 | 1.00 | 4.32 | | C |
| ATOM | 1830 | O | TYR G | 299 | 34.067 | −0.214 | 1.944 | 1.00 | 4.67 | | O |
| ATOM | 1831 | N | VAL G | 300 | 33.609 | −0.641 | −0.217 | 1.00 | 3.87 | | N |
| ATOM | 1832 | CA | VAL G | 300 | 34.121 | 0.636 | −0.696 | 1.00 | 3.57 | | C |
| ATOM | 1833 | CB | VAL G | 300 | 34.892 | 0.481 | −2.020 | 1.00 | 3.52 | | C |
| ATOM | 1834 | CG1 | VAL G | 300 | 35.278 | 1.843 | −2.576 | 1.00 | 3.44 | | C |
| ATOM | 1835 | CG2 | VAL G | 300 | 36.127 | −0.374 | −1.808 | 1.00 | 3.90 | | C |
| ATOM | 1836 | C | VAL G | 300 | 32.999 | 1.654 | −0.861 | 1.00 | 3.53 | | C |
| ATOM | 1837 | O | VAL G | 300 | 31.959 | 1.360 | −1.451 | 1.00 | 3.68 | | O |
| ATOM | 1838 | N | VAL G | 301 | 33.163 | 2.795 | −0.205 | 1.00 | 3.41 | | N |
| ATOM | 1839 | CA | VAL G | 301 | 32.321 | 3.950 | −0.451 | 1.00 | 3.36 | | C |
| ATOM | 1840 | CB | VAL G | 301 | 32.264 | 4.847 | 0.789 | 1.00 | 3.24 | | C |
| ATOM | 1841 | CG1 | VAL G | 301 | 31.738 | 6.229 | 0.428 | 1.00 | 3.76 | | C |
| ATOM | 1842 | CG2 | VAL G | 301 | 31.431 | 4.190 | 1.884 | 1.00 | 3.10 | | C |
| ATOM | 1843 | C | VAL G | 301 | 32.854 | 4.766 | −1.619 | 1.00 | 3.48 | | C |
| ATOM | 1844 | O | VAL G | 301 | 34.065 | 4.911 | −1.786 | 1.00 | 3.92 | | O |
| ATOM | 1845 | N | GLN G | 302 | 31.943 | 5.410 | −2.339 | 1.00 | 3.21 | | N |
| ATOM | 1846 | CA | GLN G | 302 | 32.285 | 6.084 | −3.578 | 1.00 | 3.01 | | C |
| ATOM | 1847 | CB | GLN G | 302 | 31.901 | 5.213 | −4.775 | 1.00 | 3.24 | | C |
| ATOM | 1848 | CG | GLN G | 302 | 31.990 | 5.924 | −6.113 | 1.00 | 5.21 | | C |
| ATOM | 1849 | CD | GLN G | 302 | 32.153 | 4.963 | −7.271 | 1.00 | 7.54 | | C |
| ATOM | 1850 | OE1 | GLN G | 302 | 32.555 | 3.814 | −7.086 | 1.00 | 7.28 | | O |
| ATOM | 1851 | NE2 | GLN G | 302 | 31.793 | 5.412 | −8.467 | 1.00 | 9.27 | | N |
| ATOM | 1852 | C | GLN G | 302 | 31.571 | 7.424 | −3.652 | 1.00 | 2.42 | | C |
| ATOM | 1853 | O | GLN G | 302 | 30.344 | 7.479 | −3.737 | 1.00 | 2.40 | | O |
| ATOM | 1854 | N | LEU G | 303 | 32.323 | 8.492 | −3.426 | 1.00 | 2.00 | | N |
| ATOM | 1855 | CA | LEU G | 303 | 31.729 | 9.802 | −3.232 | 1.00 | 2.00 | | C |
| ATOM | 1856 | CB | LEU G | 303 | 32.303 | 10.471 | −1.979 | 1.00 | 2.00 | | C |
| ATOM | 1857 | CG | LEU G | 303 | 32.332 | 9.619 | −0.706 | 1.00 | 2.00 | | C |
| ATOM | 1858 | CD1 | LEU G | 303 | 32.849 | 10.428 | 0.474 | 1.00 | 2.00 | | C |
| ATOM | 1859 | CD2 | LEU G | 303 | 30.960 | 9.033 | −0.400 | 1.00 | 2.00 | | C |
| ATOM | 1860 | C | LEU G | 303 | 31.955 | 10.674 | −4.460 | 1.00 | 2.00 | | C |
| ATOM | 1861 | O | LEU G | 303 | 32.835 | 10.391 | −5.273 | 1.00 | 2.45 | | O |
| ATOM | 1862 | N | PRO G | 304 | 31.136 | 11.723 | −4.614 | 1.00 | 2.00 | | N |
| ATOM | 1863 | CA | PRO G | 304 | 31.230 | 12.520 | −5.827 | 1.00 | 2.07 | | C |
| ATOM | 1864 | CB | PRO G | 304 | 29.999 | 13.432 | −5.754 | 1.00 | 2.00 | | C |
| ATOM | 1865 | CG | PRO G | 304 | 29.497 | 13.323 | −4.348 | 1.00 | 2.00 | | C |
| ATOM | 1866 | CD | PRO G | 304 | 29.880 | 11.966 | −3.888 | 1.00 | 2.06 | | C |
| ATOM | 1867 | C | PRO G | 304 | 32.513 | 13.340 | −5.854 | 1.00 | 2.11 | | C |
| ATOM | 1868 | O | PRO G | 304 | 32.814 | 14.030 | −4.878 | 1.00 | 2.13 | | O |
| ATOM | 1869 | N | ALA G | 305 | 33.407 | 12.935 | −6.752 | 1.00 | 30.00 | | N |
| ATOM | 1870 | CA | ALA G | 305 | 33.971 | 13.839 | −7.760 | 1.00 | 30.00 | | C |
| ATOM | 1871 | CB | ALA G | 305 | 35.427 | 13.480 | −8.043 | 1.00 | 30.00 | | C |
| ATOM | 1872 | C | ALA G | 305 | 33.151 | 13.838 | −9.059 | 1.00 | 30.00 | | C |
| ATOM | 1873 | O | ALA G | 305 | 32.743 | 12.775 | −9.530 | 1.00 | 30.00 | | O |
| ATOM | 1874 | N | TYR G | 306 | 32.752 | 15.032 | −9.522 | 1.00 | 13.49 | | N |
| ATOM | 1875 | CA | TYR G | 306 | 31.656 | 15.131 | −10.505 | 1.00 | 13.70 | | C |
| ATOM | 1876 | CB | TYR G | 306 | 30.892 | 16.462 | −10.539 | 1.00 | 13.93 | | C |
| ATOM | 1877 | CG | TYR G | 306 | 31.430 | 17.687 | −9.879 | 1.00 | 14.08 | | C |
| ATOM | 1878 | CD1 | TYR G | 306 | 32.339 | 18.519 | −10.551 | 1.00 | 14.59 | | C |
| ATOM | 1879 | CE1 | TYR G | 306 | 32.383 | 19.885 | −10.286 | 1.00 | 14.82 | | C |
| ATOM | 1880 | CZ | TYR G | 306 | 31.470 | 20.425 | −9.404 | 1.00 | 14.52 | | C |
| ATOM | 1881 | OH | TYR G | 306 | 31.626 | 21.709 | −8.943 | 1.00 | 13.02 | | O |
| ATOM | 1882 | CE2 | TYR G | 306 | 30.465 | 19.645 | −8.899 | 1.00 | 15.22 | | C |
| ATOM | 1883 | CD2 | TYR G | 306 | 30.596 | 18.296 | −8.957 | 1.00 | 14.61 | | C |
| ATOM | 1884 | C | TYR G | 306 | 32.048 | 14.998 | −11.938 | 1.00 | 13.68 | | C |
| ATOM | 1885 | O | TYR G | 306 | 33.051 | 14.374 | −12.273 | 1.00 | 13.95 | | O |
| ATOM | 1886 | N | GLY G | 307 | 31.521 | 16.002 | −12.645 | 1.00 | 13.54 | | N |
| ATOM | 1887 | CA | GLY G | 307 | 31.871 | 16.332 | −14.033 | 1.00 | 13.37 | | C |
| ATOM | 1888 | C | GLY G | 307 | 30.772 | 16.073 | −15.065 | 1.00 | 13.47 | | C |
| ATOM | 1889 | O | GLY G | 307 | 30.263 | 14.955 | −15.138 | 1.00 | 13.90 | | O |
| ATOM | 1890 | N | VAL G | 308 | 30.591 | 16.991 | −16.021 | 1.00 | 13.31 | | N |
| ATOM | 1891 | CA | VAL G | 308 | 29.883 | 16.654 | −17.276 | 1.00 | 13.30 | | C |
| ATOM | 1892 | CB | VAL G | 308 | 28.555 | 15.924 | −16.985 | 1.00 | 13.03 | | C |
| ATOM | 1893 | CG1 | VAL G | 308 | 28.183 | 15.008 | −18.138 | 1.00 | 13.62 | | C |
| ATOM | 1894 | CG2 | VAL G | 308 | 28.619 | 15.178 | −15.665 | 1.00 | 12.86 | | C |
| ATOM | 1895 | C | VAL G | 308 | 29.425 | 17.870 | −18.046 | 1.00 | 13.58 | | C |
| ATOM | 1896 | O | VAL G | 308 | 30.236 | 18.677 | −18.508 | 1.00 | 13.71 | | O |
| ATOM | 1897 | N | ILE G | 309 | 28.221 | 18.219 | −17.643 | 1.00 | 4.08 | | N |
| ATOM | 1898 | CA | ILE G | 309 | 28.140 | 19.052 | −16.487 | 1.00 | 4.84 | | C |
| ATOM | 1899 | CB | ILE G | 309 | 29.493 | 19.031 | −15.728 | 1.00 | 4.89 | | C |
| ATOM | 1900 | CG1 | ILE G | 309 | 29.924 | 17.598 | −15.412 | 1.00 | 5.85 | | C |
| ATOM | 1901 | CD1 | ILE G | 309 | 29.235 | 17.007 | −14.197 | 1.00 | 8.12 | | C |
| ATOM | 1902 | CG2 | ILE G | 309 | 29.423 | 19.879 | −14.473 | 1.00 | 4.44 | | C |
| ATOM | 1903 | C | ILE G | 309 | 28.075 | 20.346 | −17.222 | 1.00 | 5.23 | | C |
| ATOM | 1904 | O | ILE G | 309 | 28.803 | 20.526 | −18.199 | 1.00 | 5.92 | | O |
| ATOM | 1905 | N | ASP G | 310 | 26.984 | 21.057 | −17.020 | 1.00 | 5.33 | | N |
| ATOM | 1906 | CA | ASP G | 310 | 26.985 | 22.446 | −17.403 | 1.00 | 5.83 | | C |
| ATOM | 1907 | CB | ASP G | 310 | 28.432 | 22.938 | −17.554 | 1.00 | 6.21 | | C |
| ATOM | 1908 | CG | ASP G | 310 | 29.280 | 22.660 | −16.321 | 1.00 | 7.19 | | C |
| ATOM | 1909 | OD1 | ASP G | 310 | 30.432 | 22.200 | −16.480 | 1.00 | 7.74 | | O |
| ATOM | 1910 | OD2 | ASP G | 310 | 28.844 | 23.021 | −15.209 | 1.00 | 7.58 | | O |
| ATOM | 1911 | C | ASP G | 310 | 26.233 | 22.676 | −18.712 | 1.00 | 6.02 | | C |
| ATOM | 1912 | O | ASP G | 310 | 26.323 | 23.760 | −19.287 | 1.00 | 6.05 | | O |
| ATOM | 1913 | N | THR G | 311 | 25.448 | 21.696 | −19.157 | 1.00 | 6.24 | | N |
| ATOM | 1914 | CA | THR G | 311 | 24.352 | 21.982 | −20.088 | 1.00 | 6.14 | | C |
| ATOM | 1915 | CB | THR G | 311 | 23.883 | 20.720 | −20.852 | 1.00 | 5.69 | | C |
| ATOM | 1916 | OG1 | THR G | 311 | 24.925 | 19.737 | −20.850 | 1.00 | 5.34 | | O |
| ATOM | 1917 | CG2 | THR G | 311 | 23.526 | 21.069 | −22.291 | 1.00 | 7.10 | | C |
| ATOM | 1918 | C | THR G | 311 | 23.160 | 22.578 | −19.339 | 1.00 | 6.78 | | C |
| ATOM | 1919 | O | THR G | 311 | 22.863 | 22.163 | −18.218 | 1.00 | 7.33 | | O |
| ATOM | 1920 | N | PRO G | 312 | 22.414 | 23.483 | −19.992 | 1.00 | 7.00 | | N |
| ATOM | 1921 | CA | PRO G | 312 | 21.205 | 23.982 | −19.341 | 1.00 | 7.23 | | C |
| ATOM | 1922 | CB | PRO G | 312 | 20.748 | 25.113 | −20.267 | 1.00 | 7.00 | | C |
| ATOM | 1923 | CG | PRO G | 312 | 21.341 | 24.786 | −21.593 | 1.00 | 6.59 | | C |
| ATOM | 1924 | CD | PRO G | 312 | 22.649 | 24.119 | −21.300 | 1.00 | 6.99 | | C |
| ATOM | 1925 | C | PRO G | 312 | 20.138 | 22.895 | −19.263 | 1.00 | 7.69 | | C |
| ATOM | 1926 | O | PRO G | 312 | 19.973 | 22.130 | −20.213 | 1.00 | 7.55 | | O |
| ATOM | 1927 | N | CYS G | 313 | 19.469 | 22.781 | −18.120 | 1.00 | 8.10 | | N |
| ATOM | 1928 | CA | CYS G | 313 | 18.164 | 22.128 | −18.088 | 1.00 | 8.54 | | C |
| ATOM | 1929 | CB | CYS G | 313 | 18.165 | 20.883 | −17.192 | 1.00 | 8.77 | | C |
| ATOM | 1930 | SG | CYS G | 313 | 19.762 | 20.065 | −16.969 | 1.00 | 10.74 | | S |
| ATOM | 1931 | C | CYS G | 313 | 17.029 | 23.057 | −17.686 | 1.00 | 8.19 | | C |
| ATOM | 1932 | O | CYS G | 313 | 17.246 | 24.212 | −17.319 | 1.00 | 8.07 | | O |
| ATOM | 1933 | N | TRP G | 314 | 15.832 | 22.485 | −17.634 | 1.00 | 7.93 | | N |
| ATOM | 1934 | CA | TRP G | 314 | 14.663 | 23.177 | −17.125 | 1.00 | 7.80 | | C |
| ATOM | 1935 | CB | TRP G | 314 | 14.110 | 24.131 | −18.184 | 1.00 | 8.13 | | C |
| ATOM | 1936 | CG | TRP G | 314 | 13.856 | 23.474 | −19.505 | 1.00 | 9.09 | | C |
| ATOM | 1937 | CD1 | TRP G | 314 | 12.657 | 23.036 | −19.985 | 1.00 | 10.26 | | C |
| ATOM | 1938 | NE1 | TRP G | 314 | 12.807 | 22.540 | −21.257 | 1.00 | 10.86 | | N |
| ATOM | 1939 | CE2 | TRP G | 314 | 14.121 | 22.661 | −21.628 | 1.00 | 10.68 | | C |
| ATOM | 1940 | CD2 | TRP G | 314 | 14.813 | 23.243 | −20.547 | 1.00 | 10.49 | | C |
| ATOM | 1941 | CE3 | TRP G | 314 | 16.185 | 23.483 | −20.675 | 1.00 | 11.67 | | C |
| ATOM | 1942 | CZ3 | TRP G | 314 | 16.814 | 23.119 | −21.850 | 1.00 | 12.98 | | C |
| ATOM | 1943 | CH2 | TRP G | 314 | 16.104 | 22.513 | −22.894 | 1.00 | 12.74 | | C |
| ATOM | 1944 | CZ2 | TRP G | 314 | 14.758 | 22.286 | −22.807 | 1.00 | 11.73 | | C |
| ATOM | 1945 | C | TRP G | 314 | 13.603 | 22.153 | −16.742 | 1.00 | 7.34 | | C |
| ATOM | 1946 | O | TRP G | 314 | 13.578 | 21.044 | −17.275 | 1.00 | 7.47 | | O |
| ATOM | 1947 | N | LYS G | 315 | 12.783 | 22.500 | −15.757 | 1.00 | 6.75 | | N |
| ATOM | 1948 | CA | LYS G | 315 | 11.667 | 21.654 | −15.363 | 1.00 | 6.37 | | C |
| ATOM | 1949 | CB | LYS G | 315 | 11.631 | 21.475 | −13.844 | 1.00 | 6.38 | | C |
| ATOM | 1950 | CG | LYS G | 315 | 10.746 | 20.331 | −13.374 | 1.00 | 6.55 | | C |
| ATOM | 1951 | CD | LYS G | 315 | 10.695 | 20.252 | −11.854 | 1.00 | 8.01 | | C |
| ATOM | 1952 | CE | LYS G | 315 | 9.848 | 19.077 | −11.383 | 1.00 | 8.78 | | C |
| ATOM | 1953 | NZ | LYS G | 315 | 9.177 | 19.368 | −10.085 | 1.00 | 10.21 | | N |
| ATOM | 1954 | C | LYS G | 315 | 10.348 | 22.231 | −15.855 | 1.00 | 6.32 | | C |
| ATOM | 1955 | O | LYS G | 315 | 10.123 | 23.441 | −15.798 | 1.00 | 6.21 | | O |
| ATOM | 1956 | N | LEU G | 316 | 9.529 | 21.370 | −16.443 | 1.00 | 6.58 | | N |
| ATOM | 1957 | CA | LEU G | 316 | 8.273 | 21.796 | −17.027 | 1.00 | 7.02 | | C |
| ATOM | 1958 | CB | LEU G | 316 | 8.140 | 21.253 | −18.448 | 1.00 | 6.64 | | C |
| ATOM | 1959 | CG | LEU G | 316 | 6.756 | 21.379 | −19.086 | 1.00 | 6.21 | | C |
| ATOM | 1960 | CD1 | LEU G | 316 | 6.358 | 22.841 | −19.221 | 1.00 | 5.94 | | C |
| ATOM | 1961 | CD2 | LEU G | 316 | 6.730 | 20.687 | −20.439 | 1.00 | 6.57 | | C |
| ATOM | 1962 | C | LEU G | 316 | 7.116 | 21.308 | −16.172 | 1.00 | 7.77 | | C |
| ATOM | 1963 | O | LEU G | 316 | 6.942 | 20.105 | −15.976 | 1.00 | 8.47 | | O |
| ATOM | 1964 | N | HIS G | 317 | 6.363 | 22.247 | −15.613 | 1.00 | 8.05 | | N |
| ATOM | 1965 | CA | HIS G | 317 | 5.073 | 21.923 | −15.031 | 1.00 | 8.21 | | C |
| ATOM | 1966 | CB | HIS G | 317 | 4.956 | 22.485 | −13.614 | 1.00 | 9.04 | | C |
| ATOM | 1967 | CG | HIS G | 317 | 6.260 | 22.565 | −12.886 | 1.00 | 11.41 | | C |
| ATOM | 1968 | ND1 | HIS G | 317 | 6.606 | 21.691 | −11.877 | 1.00 | 13.62 | | N |
| ATOM | 1969 | CE1 | HIS G | 317 | 7.781 | 22.035 | −11.383 | 1.00 | 14.91 | | C |
| ATOM | 1970 | NE2 | HIS G | 317 | 8.208 | 23.105 | −12.029 | 1.00 | 14.25 | | N |
| ATOM | 1971 | CD2 | HIS G | 317 | 7.271 | 23.462 | −12.969 | 1.00 | 12.67 | | C |
| ATOM | 1972 | C | HIS G | 317 | 3.943 | 22.453 | −15.894 | 1.00 | 7.39 | | C |
| ATOM | 1973 | O | HIS G | 317 | 4.132 | 23.381 | −16.680 | 1.00 | 7.23 | | O |
| ATOM | 1974 | N | THR G | 318 | 2.736 | 22.006 | −15.580 | 1.00 | 7.11 | | N |
| ATOM | 1975 | CA | THR G | 318 | 1.704 | 21.826 | −16.583 | 1.00 | 6.56 | | C |
| ATOM | 1976 | CB | THR G | 318 | 1.869 | 20.474 | −17.309 | 1.00 | 6.28 | | C |
| ATOM | 1977 | OG1 | THR G | 318 | 2.413 | 20.694 | −18.616 | 1.00 | 6.33 | | O |
| ATOM | 1978 | CG2 | THR G | 318 | 0.534 | 19.761 | −17.435 | 1.00 | 6.52 | | C |
| ATOM | 1979 | C | THR G | 318 | 0.349 | 21.865 | −15.893 | 1.00 | 6.58 | | C |
| ATOM | 1980 | O | THR G | 318 | 0.104 | 21.105 | −14.958 | 1.00 | 6.67 | | O |
| ATOM | 1981 | N | SER G | 319 | −0.501 | 22.802 | −16.301 | 1.00 | 6.81 | | N |
| ATOM | 1982 | CA | SER G | 319 | −1.840 | 22.911 | −15.730 | 1.00 | 7.31 | | C |
| ATOM | 1983 | CB | SER G | 319 | −1.929 | 24.106 | −14.779 | 1.00 | 7.14 | | C |
| ATOM | 1984 | OG | SER G | 319 | −3.255 | 24.292 | −14.314 | 1.00 | 6.84 | | O |
| ATOM | 1985 | C | SER G | 319 | −2.922 | 23.006 | −16.800 | 1.00 | 7.93 | | C |
| ATOM | 1986 | O | SER G | 319 | −2.717 | 23.624 | −17.845 | 1.00 | 8.65 | | O |
| ATOM | 1987 | N | PRO G | 320 | −4.104 | 22.440 | −16.514 | 1.00 | 8.08 | | N |
| ATOM | 1988 | CA | PRO G | 320 | −5.172 | 22.399 | −17.500 | 1.00 | 8.34 | | C |
| ATOM | 1989 | CB | PRO G | 320 | −6.336 | 21.738 | −16.743 | 1.00 | 7.99 | | C |
| ATOM | 1990 | CG | PRO G | 320 | −5.908 | 21.662 | −15.304 | 1.00 | 8.01 | | C |
| ATOM | 1991 | CD | PRO G | 320 | −4.422 | 21.635 | −15.324 | 1.00 | 8.10 | | C |
| ATOM | 1992 | C | PRO G | 320 | −5.564 | 23.793 | −17.984 | 1.00 | 9.03 | | C |
| ATOM | 1993 | O | PRO G | 320 | −5.261 | 24.788 | −17.325 | 1.00 | 8.74 | | O |
| ATOM | 1994 | N | LEU G | 321 | −6.134 | 23.854 | −19.184 | 1.00 | 10.31 | | N |
| ATOM | 1995 | CA | LEU G | 321 | −6.366 | 25.113 | −19.885 | 1.00 | 11.63 | | C |
| ATOM | 1996 | CB | LEU G | 321 | −5.207 | 25.402 | −20.848 | 1.00 | 11.31 | | C |
| ATOM | 1997 | CG | LEU G | 321 | −4.949 | 26.848 | −21.291 | 1.00 | 10.97 | | C |
| ATOM | 1998 | CD1 | LEU G | 321 | −4.174 | 26.884 | −22.600 | 1.00 | 10.04 | | C |
| ATOM | 1999 | CD2 | LEU G | 321 | −6.241 | 27.639 | −21.416 | 1.00 | 10.69 | | C |
| ATOM | 2000 | C | LEU G | 321 | −7.683 | 25.027 | −20.662 | 1.00 | 12.92 | | C |
| ATOM | 2001 | O | LEU G | 321 | −7.890 | 24.095 | −21.439 | 1.00 | 13.29 | | O |
| ATOM | 2002 | N | CYS G | 322 | −8.593 | 25.964 | −20.400 | 1.00 | 13.39 | | N |
| ATOM | 2003 | CA | CYS G | 322 | −9.923 | 25.955 | −21.017 | 1.00 | 14.16 | | C |
| ATOM | 2004 | CB | CYS G | 322 | −10.994 | 25.588 | −19.990 | 1.00 | 13.72 | | C |
| ATOM | 2005 | SG | CYS G | 322 | −10.587 | 24.180 | −18.943 | 1.00 | 13.42 | | S |
| ATOM | 2006 | C | CYS G | 322 | −10.274 | 27.299 | −21.650 | 1.00 | 15.46 | | C |
| ATOM | 2007 | O | CYS G | 322 | −10.048 | 28.352 | −21.054 | 1.00 | 15.94 | | O |
| ATOM | 2008 | N | THR G | 323 | −10.985 | 27.241 | −22.773 | 1.00 | 16.70 | | N |
| ATOM | 2009 | CA | THR G | 323 | −11.694 | 28.395 | −23.326 | 1.00 | 17.84 | | C |
| ATOM | 2010 | CB | THR G | 323 | −12.389 | 28.017 | −24.647 | 1.00 | 17.50 | | C |
| ATOM | 2011 | OG1 | THR G | 323 | −12.703 | 26.618 | −24.638 | 1.00 | 15.70 | | O |
| ATOM | 2012 | CG2 | THR G | 323 | −11.486 | 28.322 | −25.831 | 1.00 | 18.05 | | C |
| ATOM | 2013 | C | THR G | 323 | −12.756 | 28.915 | −22.359 | 1.00 | 19.54 | | C |
| ATOM | 2014 | O | THR G | 323 | −13.178 | 28.188 | −21.461 | 1.00 | 19.84 | | O |
| ATOM | 2015 | N | THR G | 324 | −13.354 | 30.061 | −22.684 | 1.00 | 21.17 | | N |
| ATOM | 2016 | CA | THR G | 324 | −14.592 | 30.460 | −22.017 | 1.00 | 22.89 | | C |
| ATOM | 2017 | CB | THR G | 324 | −14.343 | 31.388 | −20.821 | 1.00 | 22.69 | | C |
| ATOM | 2018 | OG1 | THR G | 324 | −12.937 | 31.469 | −20.560 | 1.00 | 22.10 | | O |
| ATOM | 2019 | CG2 | THR G | 324 | −15.061 | 30.857 | −19.587 | 1.00 | 22.03 | | C |
| ATOM | 2020 | C | THR G | 324 | −15.712 | 31.033 | −22.885 | 1.00 | 24.39 | | C |
| ATOM | 2021 | O | THR G | 324 | −16.838 | 31.194 | −22.414 | 1.00 | 24.53 | | O |
| ATOM | 2022 | N | ASN G | 325 | −15.427 | 31.299 | −24.155 | 1.00 | 25.72 | | N |
| ATOM | 2023 | CA | ASN G | 325 | −16.490 | 31.473 | −25.144 | 1.00 | 27.01 | | C |
| ATOM | 2024 | CB | ASN G | 325 | −16.986 | 32.922 | −25.170 | 1.00 | 27.37 | | C |
| ATOM | 2025 | CG | ASN G | 325 | −18.079 | 33.184 | −24.148 | 1.00 | 28.46 | | C |
| ATOM | 2026 | OD1 | ASN G | 325 | −19.267 | 33.164 | −24.471 | 1.00 | 30.37 | | O |
| ATOM | 2027 | ND2 | ASN G | 325 | −17.678 | 33.444 | −22.909 | 1.00 | 28.78 | | N |
| ATOM | 2028 | C | ASN G | 325 | −16.075 | 31.032 | −26.543 | 1.00 | 27.36 | | C |
| ATOM | 2029 | O | ASN G | 325 | −14.887 | 30.902 | −26.837 | 1.00 | 27.94 | | O |
| ATOM | 2030 | N | SER G | 330 | −15.165 | 27.730 | −23.322 | 1.00 | 35.76 | | N |
| ATOM | 2031 | CA | SER G | 330 | −16.301 | 26.817 | −23.281 | 1.00 | 35.01 | | C |
| ATOM | 2032 | CB | SER G | 330 | −17.459 | 27.366 | −24.117 | 1.00 | 35.47 | | C |
| ATOM | 2033 | OG | SER G | 330 | −17.137 | 27.368 | −25.497 | 1.00 | 33.31 | | O |
| ATOM | 2034 | C | SER G | 330 | −15.906 | 25.431 | −23.779 | 1.00 | 34.35 | | C |
| ATOM | 2035 | O | SER G | 330 | −15.111 | 25.299 | −24.709 | 1.00 | 34.33 | | O |
| ATOM | 2036 | N | ASN G | 331 | −16.430 | 24.399 | −23.128 | 1.00 | 32.48 | | N |
| ATOM | 2037 | CA | ASN G | 331 | −16.609 | 23.102 | −23.770 | 1.00 | 30.72 | | C |
| ATOM | 2038 | CB | ASN G | 331 | −17.432 | 23.247 | −25.060 | 1.00 | 31.31 | | C |
| ATOM | 2039 | CG | ASN G | 331 | −18.924 | 23.445 | −24.801 | 1.00 | 32.11 | | C |
| ATOM | 2040 | OD1 | ASN G | 331 | −19.426 | 23.149 | −23.716 | 1.00 | 32.54 | | O |
| ATOM | 2041 | ND2 | ASN G | 331 | −19.651 | 23.849 | −25.838 | 1.00 | 31.30 | | N |
| ATOM | 2042 | C | ASN G | 331 | −15.298 | 22.357 | −24.074 | 1.00 | 28.71 | | C |
| ATOM | 2043 | O | ASN G | 331 | −15.322 | 21.324 | −24.741 | 1.00 | 29.07 | | O |
| ATOM | 2044 | N | ILE G | 332 | −14.155 | 22.896 | −23.649 | 1.00 | 25.44 | | N |
| ATOM | 2045 | CA | ILE G | 332 | −12.868 | 22.313 | −24.053 | 1.00 | 21.83 | | C |
| ATOM | 2046 | CB | ILE G | 332 | −12.546 | 22.613 | −25.536 | 1.00 | 21.42 | | C |
| ATOM | 2047 | CG1 | ILE G | 332 | −11.286 | 21.867 | −25.981 | 1.00 | 19.89 | | C |
| ATOM | 2048 | CD1 | ILE G | 332 | −11.573 | 20.576 | −26.724 | 1.00 | 17.67 | | C |
| ATOM | 2049 | CG2 | ILE G | 332 | −12.428 | 24.111 | −25.776 | 1.00 | 20.61 | | C |
| ATOM | 2050 | C | ILE G | 332 | −11.667 | 22.657 | −23.162 | 1.00 | 20.37 | | C |
| ATOM | 2051 | O | ILE G | 332 | −11.388 | 23.829 | −22.903 | 1.00 | 19.95 | | O |
| ATOM | 2052 | N | CYS G | 333 | −10.896 | 21.634 | −22.794 | 1.00 | 18.27 | | N |
| ATOM | 2053 | CA | CYS G | 333 | −9.666 | 21.842 | −22.031 | 1.00 | 16.47 | | C |
| ATOM | 2054 | CB | CYS G | 333 | −9.923 | 21.703 | −20.529 | 1.00 | 16.14 | | C |
| ATOM | 2055 | SG | CYS G | 333 | −11.374 | 22.595 | −19.936 | 1.00 | 17.09 | | S |
| ATOM | 2056 | C | CYS G | 333 | −8.480 | 20.967 | −22.450 | 1.00 | 15.73 | | C |
| ATOM | 2057 | O | CYS G | 333 | −8.652 | 19.820 | −22.871 | 1.00 | 15.43 | | O |
| ATOM | 2058 | N | LEU G | 334 | −7.298 | 21.425 | −22.047 | 1.00 | 15.06 | | N |
| ATOM | 2059 | CA | LEU G | 334 | −6.022 | 20.883 | −22.498 | 1.00 | 13.79 | | C |
| ATOM | 2060 | CB | LEU G | 334 | −5.346 | 21.857 | −23.472 | 1.00 | 13.39 | | C |
| ATOM | 2061 | CG | LEU G | 334 | −5.891 | 21.998 | −24.895 | 1.00 | 13.93 | | C |
| ATOM | 2062 | CD1 | LEU G | 334 | −4.764 | 22.327 | −25.858 | 1.00 | 14.20 | | C |
| ATOM | 2063 | CD2 | LEU G | 334 | −6.617 | 20.739 | −25.331 | 1.00 | 14.52 | | C |
| ATOM | 2064 | C | LEU G | 334 | −5.142 | 20.748 | −21.263 | 1.00 | 13.22 | | C |
| ATOM | 2065 | O | LEU G | 334 | −5.033 | 21.685 | −20.472 | 1.00 | 13.31 | | O |
| ATOM | 2066 | N | THR G | 335 | −4.417 | 19.640 | −21.169 | 1.00 | 12.58 | | N |
| ATOM | 2067 | CA | THR G | 335 | −3.277 | 19.561 | −20.268 | 1.00 | 12.17 | | C |
| ATOM | 2068 | CB | THR G | 335 | −3.612 | 18.748 | −19.001 | 1.00 | 12.02 | | C |
| ATOM | 2069 | OG1 | THR G | 335 | −5.021 | 18.815 | −18.744 | 1.00 | 12.64 | | O |
| ATOM | 2070 | CG2 | THR G | 335 | −2.858 | 19.295 | −17.803 | 1.00 | 11.47 | | C |
| ATOM | 2071 | C | THR G | 335 | −2.093 | 18.930 | −20.986 | 1.00 | 11.97 | | C |
| ATOM | 2072 | O | THR G | 335 | −2.258 | 17.980 | −21.751 | 1.00 | 11.93 | | O |
| ATOM | 2073 | N | ARG G | 336 | −0.942 | 19.586 | −20.892 | 1.00 | 11.60 | | N |
| ATOM | 2074 | CA | ARG G | 336 | 0.254 | 19.122 | −21.578 | 1.00 | 12.15 | | C |
| ATOM | 2075 | CB | ARG G | 336 | 1.293 | 20.239 | −21.640 | 1.00 | 12.16 | | C |
| ATOM | 2076 | CG | ARG G | 336 | 2.162 | 20.208 | −22.879 | 1.00 | 12.67 | | C |
| ATOM | 2077 | CD | ARG G | 336 | 2.261 | 21.590 | −23.500 | 1.00 | 12.52 | | C |
| ATOM | 2078 | NE | ARG G | 336 | 3.341 | 22.374 | −22.912 | 1.00 | 12.35 | | N |
| ATOM | 2079 | CZ | ARG G | 336 | 4.632 | 22.136 | −23.117 | 1.00 | 12.54 | | C |
| ATOM | 2080 | NH1 | ARG G | 336 | 5.006 | 21.109 | −23.867 | 1.00 | 12.38 | | N |
| ATOM | 2081 | NH2 | ARG G | 336 | 5.549 | 22.904 | −22.545 | 1.00 | 12.48 | | N |
| ATOM | 2082 | C | ARG G | 336 | 0.841 | 17.907 | −20.872 | 1.00 | 12.71 | | C |
| ATOM | 2083 | O | ARG G | 336 | 1.185 | 17.974 | −19.693 | 1.00 | 12.62 | | O |
| ATOM | 2084 | N | THR G | 337 | 0.885 | 16.779 | −21.574 | 1.00 | 13.70 | | N |
| ATOM | 2085 | CA | THR G | 337 | 1.284 | 15.514 | −20.964 | 1.00 | 14.91 | | C |
| ATOM | 2086 | CB | THR G | 337 | 1.084 | 14.331 | −21.931 | 1.00 | 15.27 | | C |
| ATOM | 2087 | OG1 | THR G | 337 | −0.264 | 14.329 | −22.421 | 1.00 | 16.65 | | O |
| ATOM | 2088 | CG2 | THR G | 337 | 1.370 | 13.015 | −21.226 | 1.00 | 15.94 | | C |
| ATOM | 2089 | C | THR G | 337 | 2.744 | 15.546 | −20.519 | 1.00 | 15.16 | | C |
| ATOM | 2090 | O | THR G | 337 | 3.069 | 15.159 | −19.396 | 1.00 | 15.60 | | O |
| ATOM | 2091 | N | ASP G | 338 | 3.623 | 15.968 | −21.424 | 1.00 | 15.15 | | N |
| ATOM | 2092 | CA | ASP G | 338 | 5.055 | 15.721 | −21.283 | 1.00 | 15.52 | | C |
| ATOM | 2093 | CB | ASP G | 338 | 5.781 | 15.935 | −22.613 | 1.00 | 16.81 | | C |
| ATOM | 2094 | CG | ASP G | 338 | 5.051 | 16.899 | −23.529 | 1.00 | 20.57 | | C |
| ATOM | 2095 | OD1 | ASP G | 338 | 5.160 | 16.744 | −24.763 | 1.00 | 23.00 | | O |
| ATOM | 2096 | OD2 | ASP G | 338 | 4.290 | 17.747 | −23.017 | 1.00 | 22.92 | | O |
| ATOM | 2097 | C | ASP G | 338 | 5.674 | 16.595 | −20.201 | 1.00 | 14.21 | | C |
| ATOM | 2098 | O | ASP G | 338 | 6.235 | 17.654 | −20.486 | 1.00 | 14.00 | | O |
| ATOM | 2099 | N | ARG G | 339 | 5.617 | 16.113 | −18.965 | 1.00 | 12.79 | | N |
| ATOM | 2100 | CA | ARG G | 339 | 6.087 | 16.877 | −17.822 | 1.00 | 11.71 | | C |
| ATOM | 2101 | CB | ARG G | 339 | 5.108 | 16.747 | −16.665 | 1.00 | 11.73 | | C |
| ATOM | 2102 | CG | ARG G | 339 | 3.773 | 17.383 | −16.931 | 1.00 | 12.49 | | C |
| ATOM | 2103 | CD | ARG G | 339 | 3.116 | 17.761 | −15.633 | 1.00 | 13.24 | | C |
| ATOM | 2104 | NE | ARG G | 339 | 1.712 | 17.370 | −15.607 | 1.00 | 12.04 | | N |
| ATOM | 2105 | CZ | ARG G | 339 | 0.844 | 17.804 | −14.703 | 1.00 | 11.74 | | C |
| ATOM | 2106 | NH1 | ARG G | 339 | 1.259 | 18.579 | −13.710 | 1.00 | 13.43 | | N |
| ATOM | 2107 | NH2 | ARG G | 339 | −0.427 | 17.428 | −14.759 | 1.00 | 9.80 | | N |
| ATOM | 2108 | C | ARG G | 339 | 7.443 | 16.388 | −17.365 | 1.00 | 11.16 | | C |
| ATOM | 2109 | O | ARG G | 339 | 7.703 | 15.185 | −17.376 | 1.00 | 11.37 | | O |
| ATOM | 2110 | N | GLY G | 340 | 8.141 | 17.269 | −16.660 | 1.00 | 10.43 | | N |
| ATOM | 2111 | CA | GLY G | 340 | 9.340 | 16.886 | −15.933 | 1.00 | 9.52 | | C |
| ATOM | 2112 | C | GLY G | 340 | 10.559 | 17.608 | −16.465 | 1.00 | 8.80 | | C |
| ATOM | 2113 | O | GLY G | 340 | 10.475 | 18.759 | −16.903 | 1.00 | 8.56 | | O |
| ATOM | 2114 | N | TRP G | 341 | 11.700 | 16.935 | −16.411 | 1.00 | 8.25 | | N |
| ATOM | 2115 | CA | TRP G | 341 | 12.965 | 17.581 | −16.704 | 1.00 | 7.98 | | C |
| ATOM | 2116 | CB | TRP G | 341 | 14.089 | 16.966 | −15.879 | 1.00 | 7.79 | | C |
| ATOM | 2117 | CG | TRP G | 341 | 14.110 | 17.460 | −14.478 | 1.00 | 7.67 | | C |
| ATOM | 2118 | CD1 | TRP G | 341 | 13.519 | 16.869 | −13.399 | 1.00 | 8.12 | | C |
| ATOM | 2119 | NE1 | TRP G | 341 | 13.686 | 17.648 | −12.283 | 1.00 | 8.60 | | N |
| ATOM | 2120 | CE2 | TRP G | 341 | 14.304 | 18.815 | −12.649 | 1.00 | 7.57 | | C |
| ATOM | 2121 | CD2 | TRP G | 341 | 14.536 | 18.754 | −14.037 | 1.00 | 7.52 | | C |
| ATOM | 2122 | CE3 | TRP G | 341 | 15.176 | 19.830 | −14.663 | 1.00 | 7.86 | | C |
| ATOM | 2123 | CZ3 | TRP G | 341 | 15.535 | 20.925 | −13.898 | 1.00 | 7.14 | | C |
| ATOM | 2124 | CH2 | TRP G | 341 | 15.279 | 20.962 | −12.521 | 1.00 | 6.39 | | C |
| ATOM | 2125 | CZ2 | TRP G | 341 | 14.663 | 19.919 | −11.880 | 1.00 | 7.30 | | C |
| ATOM | 2126 | C | TRP G | 341 | 13.286 | 17.490 | −18.180 | 1.00 | 8.26 | | C |
| ATOM | 2127 | O | TRP G | 341 | 13.320 | 16.402 | −18.755 | 1.00 | 8.35 | | O |
| ATOM | 2128 | N | TYR G | 342 | 13.400 | 18.649 | −18.811 | 1.00 | 8.44 | | N |
| ATOM | 2129 | CA | TYR G | 342 | 14.197 | 18.777 | −20.012 | 1.00 | 8.53 | | C |
| ATOM | 2130 | CB | TYR G | 342 | 13.486 | 19.670 | −21.015 | 1.00 | 8.43 | | C |
| ATOM | 2131 | CG | TYR G | 342 | 12.099 | 19.192 | −21.318 | 1.00 | 9.17 | | C |
| ATOM | 2132 | CD1 | TYR G | 342 | 11.097 | 19.299 | −20.363 | 1.00 | 10.32 | | C |
| ATOM | 2133 | CE1 | TYR G | 342 | 9.849 | 18.765 | −20.582 | 1.00 | 10.99 | | C |
| ATOM | 2134 | CZ | TYR G | 342 | 9.618 | 18.033 | −21.724 | 1.00 | 11.00 | | C |
| ATOM | 2135 | OH | TYR G | 342 | 8.365 | 17.521 | −21.964 | 1.00 | 12.82 | | O |
| ATOM | 2136 | CE2 | TYR G | 342 | 10.626 | 17.838 | −22.643 | 1.00 | 10.67 | | C |
| ATOM | 2137 | CD2 | TYR G | 342 | 11.871 | 18.365 | −22.403 | 1.00 | 10.41 | | C |
| ATOM | 2138 | C | TYR G | 342 | 15.550 | 19.359 | −19.680 | 1.00 | 8.64 | | C |
| ATOM | 2139 | O | TYR G | 342 | 15.754 | 19.906 | −18.596 | 1.00 | 8.40 | | O |
| ATOM | 2140 | N | CYS G | 343 | 16.460 | 19.284 | −20.641 | 1.00 | 8.87 | | N |
| ATOM | 2141 | CA | CYS G | 343 | 17.875 | 19.346 | −20.332 | 1.00 | 9.35 | | C |
| ATOM | 2142 | CB | CYS G | 343 | 18.190 | 18.501 | −19.092 | 1.00 | 9.17 | | C |
| ATOM | 2143 | SG | CYS G | 343 | 19.879 | 18.684 | −18.466 | 1.00 | 11.88 | | S |
| ATOM | 2144 | C | CYS G | 343 | 18.722 | 18.920 | −21.524 | 1.00 | 9.42 | | C |
| ATOM | 2145 | O | CYS G | 343 | 18.505 | 17.856 | −22.103 | 1.00 | 9.96 | | O |
| ATOM | 2146 | N | ASP G | 344 | 19.534 | 19.857 | −22.005 | 1.00 | 9.36 | | N |
| ATOM | 2147 | CA | ASP G | 344 | 20.127 | 19.778 | −23.338 | 1.00 | 9.24 | | C |
| ATOM | 2148 | CB | ASP G | 344 | 20.797 | 21.108 | −23.703 | 1.00 | 9.34 | | C |
| ATOM | 2149 | CG | ASP G | 344 | 19.874 | 22.037 | −24.474 | 1.00 | 10.72 | | C |
| ATOM | 2150 | OD1 | ASP G | 344 | 19.475 | 21.677 | −25.602 | 1.00 | 13.93 | | O |
| ATOM | 2151 | OD2 | ASP G | 344 | 19.548 | 23.127 | −23.954 | 1.00 | 11.87 | | O |
| ATOM | 2152 | C | ASP G | 344 | 21.148 | 18.651 | −23.425 | 1.00 | 9.09 | | C |
| ATOM | 2153 | O | ASP G | 344 | 22.100 | 18.605 | −22.646 | 1.00 | 9.39 | | O |
| ATOM | 2154 | N | ASN G | 345 | 21.038 | 17.843 | −24.474 | 1.00 | 9.13 | | N |
| ATOM | 2155 | CA | ASN G | 345 | 22.114 | 16.939 | −24.849 | 1.00 | 9.52 | | C |
| ATOM | 2156 | CB | ASN G | 345 | 21.589 | 15.509 | −24.978 | 1.00 | 9.61 | | C |
| ATOM | 2157 | CG | ASN G | 345 | 22.640 | 14.472 | −24.643 | 1.00 | 9.21 | | C |
| ATOM | 2158 | OD1 | ASN G | 345 | 23.121 | 14.400 | −23.512 | 1.00 | 6.21 | | O |
| ATOM | 2159 | ND2 | ASN G | 345 | 23.032 | 13.685 | −25.638 | 1.00 | 11.05 | | N |
| ATOM | 2160 | C | ASN G | 345 | 22.772 | 17.381 | −26.149 | 1.00 | 10.06 | | C |
| ATOM | 2161 | O | ASN G | 345 | 22.506 | 18.476 | −26.644 | 1.00 | 11.10 | | O |
| ATOM | 2162 | N | ALA G | 346 | 23.644 | 16.539 | −26.692 | 1.00 | 9.74 | | N |
| ATOM | 2163 | CA | ALA G | 346 | 24.410 | 16.904 | −27.877 | 1.00 | 9.58 | | C |
| ATOM | 2164 | CB | ALA G | 346 | 25.241 | 15.723 | −28.356 | 1.00 | 9.60 | | C |
| ATOM | 2165 | C | ALA G | 346 | 23.498 | 17.406 | −28.994 | 1.00 | 9.66 | | C |
| ATOM | 2166 | O | ALA G | 346 | 22.977 | 16.618 | −29.782 | 1.00 | 9.39 | | O |
| ATOM | 2167 | N | GLY G | 347 | 23.290 | 18.720 | −29.038 | 1.00 | 10.04 | | N |
| ATOM | 2168 | CA | GLY G | 347 | 22.598 | 19.358 | −30.157 | 1.00 | 10.68 | | C |
| ATOM | 2169 | C | GLY G | 347 | 21.105 | 19.098 | −30.137 | 1.00 | 10.90 | | C |
| ATOM | 2170 | O | GLY G | 347 | 20.366 | 19.569 | −31.002 | 1.00 | 11.46 | | O |
| ATOM | 2171 | N | SER G | 348 | 20.691 | 18.235 | −29.219 | 1.00 | 10.30 | | N |
| ATOM | 2172 | CA | SER G | 348 | 19.301 | 17.838 | −29.098 | 1.00 | 9.94 | | C |
| ATOM | 2173 | CB | SER G | 348 | 19.103 | 16.421 | −29.639 | 1.00 | 9.88 | | C |
| ATOM | 2174 | OG | SER G | 348 | 19.440 | 16.352 | −31.013 | 1.00 | 10.09 | | O |
| ATOM | 2175 | C | SER G | 348 | 18.950 | 17.883 | −27.624 | 1.00 | 9.60 | | C |
| ATOM | 2176 | O | SER G | 348 | 19.775 | 18.267 | −26.796 | 1.00 | 9.59 | | O |
| ATOM | 2177 | N | VAL G | 349 | 17.723 | 17.505 | −27.292 | 1.00 | 9.54 | | N |
| ATOM | 2178 | CA | VAL G | 349 | 17.254 | 17.643 | −25.925 | 1.00 | 9.56 | | C |
| ATOM | 2179 | CB | VAL G | 349 | 16.091 | 18.639 | −25.823 | 1.00 | 9.28 | | C |
| ATOM | 2180 | CG1 | VAL G | 349 | 15.814 | 18.985 | −24.360 | 1.00 | 9.24 | | C |
| ATOM | 2181 | CG2 | VAL G | 349 | 16.391 | 19.892 | −26.641 | 1.00 | 9.94 | | C |
| ATOM | 2182 | C | VAL G | 349 | 16.824 | 16.309 | −25.341 | 1.00 | 9.59 | | C |
| ATOM | 2183 | O | VAL G | 349 | 16.029 | 15.585 | −25.939 | 1.00 | 9.69 | | O |
| ATOM | 2184 | N | SER G | 350 | 17.288 | 16.029 | −24.130 | 1.00 | 9.93 | | N |
| ATOM | 2185 | CA | SER G | 350 | 16.915 | 14.800 | −23.451 | 1.00 | 10.44 | | C |
| ATOM | 2186 | CB | SER G | 350 | 18.146 | 14.108 | −22.858 | 1.00 | 10.04 | | C |
| ATOM | 2187 | OG | SER G | 350 | 18.768 | 13.261 | −23.812 | 1.00 | 8.41 | | O |
| ATOM | 2188 | C | SER G | 350 | 15.854 | 15.039 | −22.381 | 1.00 | 11.45 | | C |
| ATOM | 2189 | O | SER G | 350 | 15.882 | 16.041 | −21.665 | 1.00 | 11.58 | | O |
| ATOM | 2190 | N | PHE G | 351 | 14.893 | 14.126 | −22.318 | 1.00 | 12.28 | | N |
| ATOM | 2191 | CA | PHE G | 351 | 13.722 | 14.291 | −21.469 | 1.00 | 13.04 | | C |
| ATOM | 2192 | CB | PHE G | 351 | 12.468 | 14.485 | −22.321 | 1.00 | 12.87 | | C |
| ATOM | 2193 | CG | PHE G | 351 | 11.192 | 14.388 | −21.539 | 1.00 | 13.52 | | C |
| ATOM | 2194 | CD1 | PHE G | 351 | 11.026 | 15.127 | −20.378 | 1.00 | 13.76 | | C |
| ATOM | 2195 | CE1 | PHE G | 351 | 9.857 | 15.044 | −19.648 | 1.00 | 14.10 | | C |
| ATOM | 2196 | CZ | PHE G | 351 | 8.855 | 14.175 | −20.045 | 1.00 | 14.07 | | C |
| ATOM | 2197 | CE2 | PHE G | 351 | 9.028 | 13.395 | −21.172 | 1.00 | 14.14 | | C |
| ATOM | 2198 | CD2 | PHE G | 351 | 10.202 | 13.489 | −21.903 | 1.00 | 13.92 | | C |
| ATOM | 2199 | C | PHE G | 351 | 13.526 | 13.082 | −20.563 | 1.00 | 13.84 | | C |
| ATOM | 2200 | O | PHE G | 351 | 13.246 | 11.979 | −21.038 | 1.00 | 13.96 | | O |
| ATOM | 2201 | N | PHE G | 352 | 13.463 | 13.340 | −19.263 | 1.00 | 14.72 | | N |
| ATOM | 2202 | CA | PHE G | 352 | 13.315 | 12.275 | −18.286 | 1.00 | 15.98 | | C |
| ATOM | 2203 | CB | PHE G | 352 | 14.429 | 12.360 | −17.243 | 1.00 | 15.75 | | C |
| ATOM | 2204 | CG | PHE G | 352 | 15.720 | 12.912 | −17.780 | 1.00 | 15.51 | | C |
| ATOM | 2205 | CD1 | PHE G | 352 | 15.899 | 14.279 | −17.924 | 1.00 | 15.05 | | C |
| ATOM | 2206 | CE1 | PHE G | 352 | 17.098 | 14.793 | −18.375 | 1.00 | 14.05 | | C |
| ATOM | 2207 | CZ | PHE G | 352 | 18.126 | 13.938 | −18.719 | 1.00 | 13.28 | | C |
| ATOM | 2208 | CE2 | PHE G | 352 | 17.948 | 12.570 | −18.615 | 1.00 | 13.71 | | C |
| ATOM | 2209 | CD2 | PHE G | 352 | 16.745 | 12.064 | −18.163 | 1.00 | 14.94 | | C |
| ATOM | 2210 | C | PHE G | 352 | 11.952 | 12.360 | −17.612 | 1.00 | 17.56 | | C |
| ATOM | 2211 | O | PHE G | 352 | 11.591 | 13.401 | −17.066 | 1.00 | 17.74 | | O |
| ATOM | 2212 | N | PRO G | 353 | 11.165 | 11.280 | −17.705 | 1.00 | 19.05 | | N |
| ATOM | 2213 | CA | PRO G | 353 | 9.719 | 11.355 | −17.537 | 1.00 | 20.40 | | C |
| ATOM | 2214 | CB | PRO G | 353 | 9.220 | 10.060 | −18.189 | 1.00 | 20.26 | | C |
| ATOM | 2215 | CG | PRO G | 353 | 10.352 | 9.587 | −19.046 | 1.00 | 19.71 | | C |
| ATOM | 2216 | CD | PRO G | 353 | 11.580 | 10.012 | −18.325 | 1.00 | 18.97 | | C |
| ATOM | 2217 | C | PRO G | 353 | 9.291 | 11.404 | −16.072 | 1.00 | 21.94 | | C |
| ATOM | 2218 | O | PRO G | 353 | 8.222 | 11.935 | −15.768 | 1.00 | 22.29 | | O |
| ATOM | 2219 | N | GLN G | 354 | 10.105 | 10.851 | −15.175 | 1.00 | 23.38 | | N |
| ATOM | 2220 | CA | GLN G | 354 | 9.731 | 10.792 | −13.761 | 1.00 | 24.64 | | C |
| ATOM | 2221 | CB | GLN G | 354 | 9.107 | 9.440 | −13.400 | 1.00 | 24.93 | | C |
| ATOM | 2222 | CG | GLN G | 354 | 9.407 | 8.322 | −14.376 | 1.00 | 26.00 | | C |
| ATOM | 2223 | CD | GLN G | 354 | 10.859 | 7.909 | −14.354 | 1.00 | 25.92 | | C |
| ATOM | 2224 | OE1 | GLN G | 354 | 11.380 | 7.473 | −13.327 | 1.00 | 25.67 | | O |
| ATOM | 2225 | NE2 | GLN G | 354 | 11.521 | 8.030 | −15.498 | 1.00 | 24.27 | | N |
| ATOM | 2226 | C | GLN G | 354 | 10.823 | 11.177 | −12.759 | 1.00 | 24.85 | | C |
| ATOM | 2227 | O | GLN G | 354 | 12.009 | 10.943 | −12.989 | 1.00 | 24.51 | | O |
| ATOM | 2228 | N | ALA G | 355 | 10.392 | 11.709 | −11.618 | 1.00 | 25.22 | | N |
| ATOM | 2229 | CA | ALA G | 355 | 11.296 | 12.264 | −10.619 | 1.00 | 25.34 | | C |
| ATOM | 2230 | CB | ALA G | 355 | 10.565 | 12.449 | −9.296 | 1.00 | 25.09 | | C |
| ATOM | 2231 | C | ALA G | 355 | 12.531 | 11.390 | −10.427 | 1.00 | 24.92 | | C |
| ATOM | 2232 | O | ALA G | 355 | 13.646 | 11.791 | −10.766 | 1.00 | 25.23 | | O |
| ATOM | 2233 | N | GLU G | 356 | 12.339 | 10.258 | −9.755 | 1.00 | 23.93 | | N |
| ATOM | 2234 | CA | GLU G | 356 | 13.282 | 9.141 | −9.803 | 1.00 | 22.77 | | C |
| ATOM | 2235 | CB | GLU G | 356 | 12.642 | 7.925 | −10.474 | 1.00 | 23.22 | | C |
| ATOM | 2236 | CG | GLU G | 356 | 11.489 | 7.338 | −9.681 | 1.00 | 26.33 | | C |
| ATOM | 2237 | CD | GLU G | 356 | 11.607 | 7.632 | −8.199 | 1.00 | 30.93 | | C |
| ATOM | 2238 | OE1 | GLU G | 356 | 11.148 | 8.711 | −7.764 | 1.00 | 31.97 | | O |
| ATOM | 2239 | OE2 | GLU G | 356 | 12.276 | 6.846 | −7.494 | 1.00 | 32.21 | | O |
| ATOM | 2240 | C | GLU G | 356 | 14.591 | 9.493 | −10.492 | 1.00 | 21.31 | | C |
| ATOM | 2241 | O | GLU G | 356 | 15.656 | 9.462 | −9.876 | 1.00 | 21.01 | | O |
| ATOM | 2242 | N | THR G | 357 | 14.511 | 9.752 | −11.791 | 1.00 | 19.51 | | N |
| ATOM | 2243 | CA | THR G | 357 | 15.690 | 9.787 | −12.639 | 1.00 | 17.55 | | C |
| ATOM | 2244 | CB | THR G | 357 | 15.305 | 9.928 | −14.114 | 1.00 | 17.30 | | C |
| ATOM | 2245 | OG1 | THR G | 357 | 13.908 | 9.660 | −14.273 | 1.00 | 17.32 | | O |
| ATOM | 2246 | CG2 | THR G | 357 | 16.099 | 8.958 | −14.957 | 1.00 | 17.72 | | C |
| ATOM | 2247 | C | THR G | 357 | 16.596 | 10.949 | −12.262 | 1.00 | 16.62 | | C |
| ATOM | 2248 | O | THR G | 357 | 17.761 | 10.991 | −12.655 | 1.00 | 17.29 | | O |
| ATOM | 2249 | N | CYS G | 358 | 16.028 | 11.935 | −11.579 | 1.00 | 14.88 | | N |
| ATOM | 2250 | CA | CYS G | 358 | 16.684 | 13.225 | −11.440 | 1.00 | 13.15 | | C |
| ATOM | 2251 | CB | CYS G | 358 | 16.025 | 14.262 | −12.349 | 1.00 | 13.04 | | C |
| ATOM | 2252 | SG | CYS G | 358 | 16.175 | 13.900 | −14.114 | 1.00 | 14.02 | | S |
| ATOM | 2253 | C | CYS G | 358 | 16.711 | 13.714 | −9.997 | 1.00 | 12.08 | | C |
| ATOM | 2254 | O | CYS G | 358 | 15.705 | 13.661 | −9.289 | 1.00 | 12.15 | | O |
| ATOM | 2255 | N | LYS G | 359 | 17.890 | 14.140 | −9.554 | 1.00 | 11.35 | | N |
| ATOM | 2256 | CA | LYS G | 359 | 18.024 | 14.888 | −8.311 | 1.00 | 11.01 | | C |
| ATOM | 2257 | CB | LYS G | 359 | 18.968 | 14.165 | −7.353 | 1.00 | 11.16 | | C |
| ATOM | 2258 | CG | LYS G | 359 | 19.450 | 12.824 | −7.857 | 1.00 | 13.28 | | C |
| ATOM | 2259 | CD | LYS G | 359 | 19.029 | 11.720 | −6.912 | 1.00 | 16.11 | | C |
| ATOM | 2260 | CE | LYS G | 359 | 19.561 | 10.378 | −7.366 | 1.00 | 17.91 | | C |
| ATOM | 2261 | NZ | LYS G | 359 | 18.972 | 9.264 | −6.575 | 1.00 | 20.99 | | N |
| ATOM | 2262 | C | LYS G | 359 | 18.561 | 16.282 | −8.584 | 1.00 | 10.46 | | C |
| ATOM | 2263 | O | LYS G | 359 | 19.145 | 16.533 | −9.637 | 1.00 | 10.70 | | O |
| ATOM | 2264 | N | VAL G | 360 | 18.526 | 17.128 | −7.564 | 1.00 | 9.99 | | N |
| ATOM | 2265 | CA | VAL G | 360 | 18.797 | 18.538 | −7.763 | 1.00 | 9.84 | | C |
| ATOM | 2266 | CB | VAL G | 360 | 17.573 | 19.270 | −8.346 | 1.00 | 9.42 | | C |
| ATOM | 2267 | CG1 | VAL G | 360 | 16.360 | 19.095 | −7.442 | 1.00 | 10.07 | | C |
| ATOM | 2268 | CG2 | VAL G | 360 | 17.886 | 20.740 | −8.572 | 1.00 | 9.56 | | C |
| ATOM | 2269 | C | VAL G | 360 | 19.259 | 19.211 | −6.478 | 1.00 | 9.98 | | C |
| ATOM | 2270 | O | VAL G | 360 | 18.707 | 18.970 | −5.405 | 1.00 | 9.96 | | O |
| ATOM | 2271 | N | GLN G | 361 | 20.372 | 19.931 | −6.578 | 1.00 | 10.39 | | N |
| ATOM | 2272 | CA | GLN G | 361 | 20.979 | 20.583 | −5.427 | 1.00 | 11.53 | | C |
| ATOM | 2273 | CB | GLN G | 361 | 22.220 | 19.809 | −4.971 | 1.00 | 12.03 | | C |
| ATOM | 2274 | CG | GLN G | 361 | 22.907 | 20.381 | −3.737 | 1.00 | 16.44 | | C |
| ATOM | 2275 | CD | GLN G | 361 | 23.241 | 19.314 | −2.714 | 1.00 | 25.16 | | C |
| ATOM | 2276 | OE1 | GLN G | 361 | 24.398 | 18.921 | −2.564 | 1.00 | 29.01 | | O |
| ATOM | 2277 | NE2 | GLN G | 361 | 22.220 | 18.812 | −2.029 | 1.00 | 27.20 | | N |
| ATOM | 2278 | C | GLN G | 361 | 21.373 | 22.005 | −5.794 | 1.00 | 11.15 | | C |
| ATOM | 2279 | O | GLN G | 361 | 22.354 | 22.221 | −6.506 | 1.00 | 11.80 | | O |
| ATOM | 2280 | N | SER G | 362 | 20.611 | 22.976 | −5.308 | 1.00 | 10.90 | | N |
| ATOM | 2281 | CA | SER G | 362 | 20.759 | 24.332 | −5.798 | 1.00 | 10.30 | | C |
| ATOM | 2282 | CB | SER G | 362 | 22.208 | 24.792 | −5.625 | 1.00 | 10.47 | | C |
| ATOM | 2283 | OG | SER G | 362 | 22.406 | 26.082 | −6.179 | 1.00 | 11.17 | | O |
| ATOM | 2284 | C | SER G | 362 | 20.385 | 24.348 | −7.272 | 1.00 | 9.58 | | C |
| ATOM | 2285 | O | SER G | 362 | 19.409 | 23.717 | −7.677 | 1.00 | 9.48 | | O |
| ATOM | 2286 | N | ASN G | 363 | 21.256 | 24.926 | −8.089 | 1.00 | 9.12 | | N |
| ATOM | 2287 | CA | ASN G | 363 | 21.023 | 24.985 | −9.525 | 1.00 | 8.73 | | C |
| ATOM | 2288 | CB | ASN G | 363 | 21.537 | 26.308 | −10.085 | 1.00 | 9.01 | | C |
| ATOM | 2289 | CG | ASN G | 363 | 23.018 | 26.497 | −9.849 | 1.00 | 9.29 | | C |
| ATOM | 2290 | OD1 | ASN G | 363 | 23.681 | 25.636 | −9.270 | 1.00 | 9.82 | | O |
| ATOM | 2291 | ND2 | ASN G | 363 | 23.555 | 27.615 | −10.323 | 1.00 | 10.08 | | N |
| ATOM | 2292 | C | ASN G | 363 | 21.701 | 23.822 | −10.237 | 1.00 | 8.12 | | C |
| ATOM | 2293 | O | ASN G | 363 | 21.961 | 23.881 | −11.439 | 1.00 | 8.10 | | O |
| ATOM | 2294 | N | ARG G | 364 | 22.103 | 22.828 | −9.453 | 1.00 | 7.20 | | N |
| ATOM | 2295 | CA | ARG G | 364 | 22.733 | 21.628 | −9.984 | 1.00 | 6.82 | | C |
| ATOM | 2296 | CB | ARG G | 364 | 23.843 | 21.154 | −9.044 | 1.00 | 6.91 | | C |
| ATOM | 2297 | CG | ARG G | 364 | 25.110 | 20.721 | −9.754 | 1.00 | 7.92 | | C |
| ATOM | 2298 | CD | ARG G | 364 | 25.588 | 21.797 | −10.709 | 1.00 | 8.20 | | C |
| ATOM | 2299 | NE | ARG G | 364 | 25.809 | 21.275 | −12.053 | 1.00 | 8.70 | | N |
| ATOM | 2300 | CZ | ARG G | 364 | 26.690 | 21.777 | −12.910 | 1.00 | 8.88 | | C |
| ATOM | 2301 | NH1 | ARG G | 364 | 27.407 | 22.843 | −12.578 | 1.00 | 8.57 | | N |
| ATOM | 2302 | NH2 | ARG G | 364 | 26.840 | 21.228 | −14.107 | 1.00 | 10.59 | | N |
| ATOM | 2303 | C | ARG G | 364 | 21.703 | 20.519 | −10.166 | 1.00 | 6.58 | | C |
| ATOM | 2304 | O | ARG G | 364 | 20.980 | 20.173 | −9.233 | 1.00 | 6.65 | | O |
| ATOM | 2305 | N | VAL G | 365 | 21.719 | 19.897 | −11.341 | 1.00 | 6.64 | | N |
| ATOM | 2306 | CA | VAL G | 365 | 20.802 | 18.805 | −11.658 | 1.00 | 6.54 | | C |
| ATOM | 2307 | CB | VAL G | 365 | 19.823 | 19.208 | −12.782 | 1.00 | 5.95 | | C |
| ATOM | 2308 | CG1 | VAL G | 365 | 19.069 | 17.993 | −13.302 | 1.00 | 5.48 | | C |
| ATOM | 2309 | CG2 | VAL G | 365 | 18.861 | 20.277 | −12.291 | 1.00 | 6.22 | | C |
| ATOM | 2310 | C | VAL G | 365 | 21.569 | 17.549 | −12.089 | 1.00 | 7.35 | | C |
| ATOM | 2311 | O | VAL G | 365 | 22.611 | 17.641 | −12.740 | 1.00 | 8.33 | | O |
| ATOM | 2312 | N | PHE G | 366 | 21.009 | 16.380 | −11.781 | 1.00 | 7.37 | | N |
| ATOM | 2313 | CA | PHE G | 366 | 21.654 | 15.097 | −12.072 | 1.00 | 7.44 | | C |
| ATOM | 2314 | CB | PHE G | 366 | 22.371 | 14.567 | −10.822 | 1.00 | 7.30 | | C |
| ATOM | 2315 | CG | PHE G | 366 | 23.464 | 15.468 | −10.314 | 1.00 | 6.70 | | C |
| ATOM | 2316 | CD1 | PHE G | 366 | 23.160 | 16.576 | −9.538 | 1.00 | 6.70 | | C |
| ATOM | 2317 | CE1 | PHE G | 366 | 24.162 | 17.406 | −9.068 | 1.00 | 5.94 | | C |
| ATOM | 2318 | CZ | PHE G | 366 | 25.487 | 17.053 | −9.244 | 1.00 | 5.71 | | C |
| ATOM | 2319 | CE2 | PHE G | 366 | 25.804 | 15.897 | −9.935 | 1.00 | 5.96 | | C |
| ATOM | 2320 | CD2 | PHE G | 366 | 24.793 | 15.094 | −10.437 | 1.00 | 6.52 | | C |
| ATOM | 2321 | C | PHE G | 366 | 20.626 | 14.059 | −12.546 | 1.00 | 7.45 | | C |
| ATOM | 2322 | O | PHE G | 366 | 19.770 | 13.635 | −11.769 | 1.00 | 7.45 | | O |
| ATOM | 2323 | N | CYS G | 367 | 20.783 | 13.569 | −13.777 | 1.00 | 8.00 | | N |
| ATOM | 2324 | CA | CYS G | 367 | 19.838 | 12.602 | −14.369 | 1.00 | 8.90 | | C |
| ATOM | 2325 | CB | CYS G | 367 | 18.879 | 13.313 | −15.335 | 1.00 | 9.46 | | C |
| ATOM | 2326 | SG | CYS G | 367 | 17.966 | 14.707 | −14.624 | 1.00 | 10.79 | | S |
| ATOM | 2327 | C | CYS G | 367 | 20.563 | 11.445 | −15.089 | 1.00 | 9.09 | | C |
| ATOM | 2328 | O | CYS G | 367 | 21.751 | 11.556 | −15.396 | 1.00 | 9.24 | | O |
| ATOM | 2329 | N | ASP G | 368 | 19.864 | 10.330 | −15.323 | 1.00 | 9.54 | | N |
| ATOM | 2330 | CA | ASP G | 368 | 20.480 | 9.110 | −15.890 | 1.00 | 10.55 | | C |
| ATOM | 2331 | CB | ASP G | 368 | 20.253 | 7.906 | −14.962 | 1.00 | 10.53 | | C |
| ATOM | 2332 | CG | ASP G | 368 | 21.043 | 6.674 | −15.386 | 1.00 | 11.11 | | C |
| ATOM | 2333 | OD1 | ASP G | 368 | 21.241 | 6.475 | −16.603 | 1.00 | 11.42 | | O |
| ATOM | 2334 | OD2 | ASP G | 368 | 21.441 | 5.892 | −14.497 | 1.00 | 11.35 | | O |
| ATOM | 2335 | C | ASP G | 368 | 19.947 | 8.774 | −17.285 | 1.00 | 11.45 | | C |
| ATOM | 2336 | O | ASP G | 368 | 19.035 | 7.958 | −17.422 | 1.00 | 12.05 | | O |
| ATOM | 2337 | N | THR G | 369 | 20.606 | 9.281 | −18.321 | 1.00 | 12.41 | | N |
| ATOM | 2338 | CA | THR G | 369 | 20.367 | 8.772 | −19.666 | 1.00 | 13.00 | | C |
| ATOM | 2339 | CB | THR G | 369 | 21.685 | 8.338 | −20.361 | 1.00 | 12.69 | | C |
| ATOM | 2340 | OG1 | THR G | 369 | 22.580 | 9.456 | −20.444 | 1.00 | 11.72 | | O |
| ATOM | 2341 | CG2 | THR G | 369 | 21.409 | 7.813 | −21.765 | 1.00 | 13.33 | | C |
| ATOM | 2342 | C | THR G | 369 | 19.406 | 7.585 | −19.574 | 1.00 | 14.13 | | C |
| ATOM | 2343 | O | THR G | 369 | 18.260 | 7.681 | −20.008 | 1.00 | 14.72 | | O |
| ATOM | 2344 | N | MET G | 370 | 19.790 | 6.577 | −18.795 | 1.00 | 15.25 | | N |
| ATOM | 2345 | CA | MET G | 370 | 19.286 | 5.221 | −18.993 | 1.00 | 16.60 | | C |
| ATOM | 2346 | CB | MET G | 370 | 19.132 | 4.486 | −17.664 | 1.00 | 17.42 | | C |
| ATOM | 2347 | CG | MET G | 370 | 19.948 | 3.204 | −17.580 | 1.00 | 20.31 | | C |
| ATOM | 2348 | SD | MET G | 370 | 19.542 | 1.991 | −18.855 | 1.00 | 26.43 | | S |
| ATOM | 2349 | CE | MET G | 370 | 20.369 | 2.694 | −20.281 | 1.00 | 24.07 | | C |
| ATOM | 2350 | C | MET G | 370 | 17.997 | 5.144 | −19.811 | 1.00 | 16.54 | | C |
| ATOM | 2351 | O | MET G | 370 | 17.975 | 4.516 | −20.870 | 1.00 | 16.74 | | O |
| ATOM | 2352 | N | ASN G | 371 | 16.923 | 5.762 | −19.327 | 1.00 | 16.35 | | N |
| ATOM | 2353 | CA | ASN G | 371 | 15.648 | 5.694 | −20.042 | 1.00 | 16.07 | | C |
| ATOM | 2354 | CB | ASN G | 371 | 14.554 | 5.050 | −19.184 | 1.00 | 16.41 | | C |
| ATOM | 2355 | CG | ASN G | 371 | 14.652 | 3.531 | −19.153 | 1.00 | 16.71 | | C |
| ATOM | 2356 | OD1 | ASN G | 371 | 14.117 | 2.843 | −20.025 | 1.00 | 15.75 | | O |
| ATOM | 2357 | ND2 | ASN G | 371 | 15.228 | 3.003 | −18.080 | 1.00 | 17.76 | | N |
| ATOM | 2358 | C | ASN G | 371 | 15.160 | 7.004 | −20.661 | 1.00 | 15.55 | | C |
| ATOM | 2359 | O | ASN G | 371 | 14.132 | 7.023 | −21.334 | 1.00 | 15.84 | | O |
| ATOM | 2360 | N | SER G | 372 | 16.006 | 8.030 | −20.612 | 1.00 | 14.76 | | N |
| ATOM | 2361 | CA | SER G | 372 | 15.784 | 9.270 | −21.364 | 1.00 | 13.91 | | C |
| ATOM | 2362 | CB | SER G | 372 | 17.101 | 10.025 | −21.561 | 1.00 | 14.04 | | C |
| ATOM | 2363 | OG | SER G | 372 | 17.984 | 9.303 | −22.405 | 1.00 | 15.09 | | O |
| ATOM | 2364 | C | SER G | 372 | 15.111 | 9.064 | −22.717 | 1.00 | 13.07 | | C |
| ATOM | 2365 | O | SER G | 372 | 15.417 | 8.111 | −23.437 | 1.00 | 13.02 | | O |
| ATOM | 2366 | N | LEU G | 373 | 14.347 | 10.072 | −23.130 | 1.00 | 12.23 | | N |
| ATOM | 2367 | CA | LEU G | 373 | 13.882 | 10.192 | −24.508 | 1.00 | 11.45 | | C |
| ATOM | 2368 | CB | LEU G | 373 | 12.358 | 10.365 | −24.534 | 1.00 | 11.43 | | C |
| ATOM | 2369 | CG | LEU G | 373 | 11.464 | 9.123 | −24.683 | 1.00 | 11.04 | | C |
| ATOM | 2370 | CD1 | LEU G | 373 | 11.965 | 7.948 | −23.852 | 1.00 | 11.76 | | C |
| ATOM | 2371 | CD2 | LEU G | 373 | 10.020 | 9.434 | −24.330 | 1.00 | 10.49 | | C |
| ATOM | 2372 | C | LEU G | 373 | 14.550 | 11.392 | −25.176 | 1.00 | 11.17 | | C |
| ATOM | 2373 | O | LEU G | 373 | 14.658 | 12.460 | −24.574 | 1.00 | 11.27 | | O |
| ATOM | 2374 | N | THR G | 374 | 15.011 | 11.208 | −26.411 | 1.00 | 10.70 | | N |
| ATOM | 2375 | CA | THR G | 374 | 15.791 | 12.235 | −27.102 | 1.00 | 10.23 | | C |
| ATOM | 2376 | CB | THR G | 374 | 16.998 | 11.634 | −27.863 | 1.00 | 10.24 | | C |
| ATOM | 2377 | OG1 | THR G | 374 | 17.406 | 10.410 | −27.239 | 1.00 | 11.25 | | O |
| ATOM | 2378 | CG2 | THR G | 374 | 18.168 | 12.612 | −27.872 | 1.00 | 10.52 | | C |
| ATOM | 2379 | C | THR G | 374 | 14.934 | 13.032 | −28.079 | 1.00 | 9.54 | | C |
| ATOM | 2380 | O | THR G | 374 | 14.316 | 12.468 | −28.983 | 1.00 | 9.70 | | O |
| ATOM | 2381 | N | LEU G | 375 | 14.965 | 14.352 | −27.935 | 1.00 | 8.83 | | N |
| ATOM | 2382 | CA | LEU G | 375 | 14.013 | 15.221 | −28.611 | 1.00 | 7.83 | | C |
| ATOM | 2383 | CB | LEU G | 375 | 12.949 | 15.719 | −27.631 | 1.00 | 7.38 | | C |
| ATOM | 2384 | CG | LEU G | 375 | 12.160 | 14.689 | −26.824 | 1.00 | 5.06 | | C |
| ATOM | 2385 | CD1 | LEU G | 375 | 11.352 | 15.385 | −25.741 | 1.00 | 2.41 | | C |
| ATOM | 2386 | CD2 | LEU G | 375 | 11.246 | 13.885 | −27.734 | 1.00 | 4.31 | | C |
| ATOM | 2387 | C | LEU G | 375 | 14.728 | 16.416 | −29.222 | 1.00 | 7.79 | | C |
| ATOM | 2388 | O | LEU G | 375 | 15.743 | 16.872 | −28.696 | 1.00 | 7.92 | | O |
| ATOM | 2389 | N | PRO G | 376 | 14.102 | 17.026 | −30.234 | 1.00 | 7.51 | | N |
| ATOM | 2390 | CA | PRO G | 376 | 14.606 | 18.224 | −30.892 | 1.00 | 7.90 | | C |
| ATOM | 2391 | CB | PRO G | 376 | 13.837 | 18.238 | −32.211 | 1.00 | 7.90 | | C |
| ATOM | 2392 | CG | PRO G | 376 | 12.558 | 17.556 | −31.891 | 1.00 | 7.97 | | C |
| ATOM | 2393 | CD | PRO G | 376 | 12.913 | 16.481 | −30.912 | 1.00 | 7.40 | | C |
| ATOM | 2394 | C | PRO G | 376 | 14.294 | 19.486 | −30.095 | 1.00 | 8.35 | | C |
| ATOM | 2395 | O | PRO G | 376 | 13.205 | 19.611 | −29.533 | 1.00 | 8.71 | | O |
| ATOM | 2396 | N | SER G | 377 | 15.206 | 20.451 | −30.137 | 1.00 | 9.11 | | N |
| ATOM | 2397 | CA | SER G | 377 | 14.990 | 21.743 | −29.494 | 1.00 | 9.91 | | C |
| ATOM | 2398 | CB | SER G | 377 | 16.155 | 22.691 | −29.792 | 1.00 | 10.23 | | C |
| ATOM | 2399 | OG | SER G | 377 | 17.300 | 21.973 | −30.222 | 1.00 | 10.90 | | O |
| ATOM | 2400 | C | SER G | 377 | 13.673 | 22.377 | −29.937 | 1.00 | 10.14 | | C |
| ATOM | 2401 | O | SER G | 377 | 13.295 | 23.444 | −29.450 | 1.00 | 10.08 | | O |
| ATOM | 2402 | N | GLU G | 378 | 13.011 | 21.749 | −30.906 | 1.00 | 10.71 | | N |
| ATOM | 2403 | CA | GLU G | 378 | 11.714 | 22.222 | −31.382 | 1.00 | 11.40 | | C |
| ATOM | 2404 | CB | GLU G | 378 | 11.357 | 21.569 | −32.721 | 1.00 | 11.65 | | C |
| ATOM | 2405 | CG | GLU G | 378 | 11.978 | 22.250 | −33.933 | 1.00 | 13.46 | | C |
| ATOM | 2406 | CD | GLU G | 378 | 13.490 | 22.160 | −33.938 | 1.00 | 16.20 | | C |
| ATOM | 2407 | OE1 | GLU G | 378 | 14.046 | 21.522 | −33.021 | 1.00 | 15.73 | | O |
| ATOM | 2408 | OE2 | GLU G | 378 | 14.121 | 22.732 | −34.852 | 1.00 | 18.38 | | O |
| ATOM | 2409 | C | GLU G | 378 | 10.623 | 21.944 | −30.353 | 1.00 | 11.27 | | C |
| ATOM | 2410 | O | GLU G | 378 | 9.561 | 22.565 | −30.377 | 1.00 | 11.28 | | O |
| ATOM | 2411 | N | VAL G | 379 | 10.909 | 21.031 | −29.430 | 1.00 | 11.31 | | N |
| ATOM | 2412 | CA | VAL G | 379 | 10.053 | 20.819 | −28.269 | 1.00 | 11.59 | | C |
| ATOM | 2413 | CB | VAL G | 379 | 10.737 | 19.926 | −27.216 | 1.00 | 11.27 | | C |
| ATOM | 2414 | CG1 | VAL G | 379 | 10.100 | 20.131 | −25.850 | 1.00 | 11.60 | | C |
| ATOM | 2415 | CG2 | VAL G | 379 | 10.667 | 18.465 | −27.631 | 1.00 | 11.54 | | C |
| ATOM | 2416 | C | VAL G | 379 | 9.665 | 22.144 | −27.620 | 1.00 | 12.16 | | C |
| ATOM | 2417 | O | VAL G | 379 | 8.541 | 22.307 | −27.146 | 1.00 | 12.51 | | O |
| ATOM | 2418 | N | ASN G | 380 | 10.587 | 23.100 | −27.634 | 1.00 | 12.98 | | N |
| ATOM | 2419 | CA | ASN G | 380 | 10.415 | 24.335 | −26.878 | 1.00 | 14.00 | | C |
| ATOM | 2420 | CB | ASN G | 380 | 11.703 | 25.156 | −26.884 | 1.00 | 14.59 | | C |
| ATOM | 2421 | CG | ASN G | 380 | 12.829 | 24.474 | −26.138 | 1.00 | 17.09 | | C |
| ATOM | 2422 | OD1 | ASN G | 380 | 13.073 | 24.764 | −24.966 | 1.00 | 20.41 | | O |
| ATOM | 2423 | ND2 | ASN G | 380 | 13.432 | 23.471 | −26.767 | 1.00 | 18.86 | | N |
| ATOM | 2424 | C | ASN G | 380 | 9.242 | 25.192 | −27.349 | 1.00 | 13.96 | | C |
| ATOM | 2425 | O | ASN G | 380 | 8.640 | 25.915 | −26.555 | 1.00 | 14.38 | | O |
| ATOM | 2426 | N | LEU G | 381 | 8.994 | 25.197 | −28.655 | 1.00 | 13.61 | | N |
| ATOM | 2427 | CA | LEU G | 381 | 7.918 | 26.006 | −29.220 | 1.00 | 13.72 | | C |
| ATOM | 2428 | CB | LEU G | 381 | 7.854 | 25.829 | −30.737 | 1.00 | 13.79 | | C |
| ATOM | 2429 | CG | LEU G | 381 | 9.183 | 25.514 | −31.424 | 1.00 | 14.92 | | C |
| ATOM | 2430 | CD1 | LEU G | 381 | 8.969 | 25.231 | −32.902 | 1.00 | 15.56 | | C |
| ATOM | 2431 | CD2 | LEU G | 381 | 10.171 | 26.655 | −31.230 | 1.00 | 15.78 | | C |
| ATOM | 2432 | C | LEU G | 381 | 6.590 | 25.603 | −28.600 | 1.00 | 13.62 | | C |
| ATOM | 2433 | O | LEU G | 381 | 5.600 | 26.331 | −28.682 | 1.00 | 13.57 | | O |
| ATOM | 2434 | N | CYS G | 382 | 6.590 | 24.440 | −27.963 | 1.00 | 13.47 | | N |
| ATOM | 2435 | CA | CYS G | 382 | 5.383 | 23.876 | −27.392 | 1.00 | 13.57 | | C |
| ATOM | 2436 | CB | CYS G | 382 | 5.572 | 22.380 | −27.161 | 1.00 | 13.62 | | C |
| ATOM | 2437 | SG | CYS G | 382 | 4.877 | 21.350 | −28.457 | 1.00 | 15.64 | | S |
| ATOM | 2438 | C | CYS G | 382 | 5.019 | 24.572 | −26.085 | 1.00 | 13.33 | | C |
| ATOM | 2439 | O | CYS G | 382 | 3.861 | 24.561 | −25.669 | 1.00 | 13.57 | | O |
| ATOM | 2440 | N | ASN G | 383 | 5.999 | 25.236 | −25.480 | 1.00 | 13.21 | | N |
| ATOM | 2441 | CA | ASN G | 383 | 5.736 | 26.114 | −24.345 | 1.00 | 13.22 | | C |
| ATOM | 2442 | CB | ASN G | 383 | 7.046 | 26.621 | −23.742 | 1.00 | 13.18 | | C |
| ATOM | 2443 | CG | ASN G | 383 | 7.979 | 25.498 | −23.349 | 1.00 | 13.42 | | C |
| ATOM | 2444 | OD1 | ASN G | 383 | 7.560 | 24.505 | −22.755 | 1.00 | 13.68 | | O |
| ATOM | 2445 | ND2 | ASN G | 383 | 9.255 | 25.648 | −23.682 | 1.00 | 14.32 | | N |
| ATOM | 2446 | C | ASN G | 383 | 4.872 | 27.298 | −24.747 | 1.00 | 13.28 | | C |
| ATOM | 2447 | O | ASN G | 383 | 4.229 | 27.925 | −23.906 | 1.00 | 13.50 | | O |
| ATOM | 2448 | N | VAL G | 384 | 5.000 | 27.699 | −26.005 | 1.00 | 13.24 | | N |
| ATOM | 2449 | CA | VAL G | 384 | 4.313 | 28.878 | −26.496 | 1.00 | 13.29 | | C |
| ATOM | 2450 | CB | VAL G | 384 | 5.234 | 29.751 | −27.370 | 1.00 | 13.28 | | C |
| ATOM | 2451 | CG1 | VAL G | 384 | 4.414 | 30.627 | −28.302 | 1.00 | 13.73 | | C |
| ATOM | 2452 | CG2 | VAL G | 384 | 6.145 | 30.599 | −26.495 | 1.00 | 12.76 | | C |
| ATOM | 2453 | C | VAL G | 384 | 3.045 | 28.514 | −27.261 | 1.00 | 13.57 | | C |
| ATOM | 2454 | O | VAL G | 384 | 2.050 | 29.233 | −27.188 | 1.00 | 13.31 | | O |
| ATOM | 2455 | N | ASP G | 385 | 3.056 | 27.383 | −27.960 | 1.00 | 14.07 | | N |
| ATOM | 2456 | CA | ASP G | 385 | 1.999 | 27.121 | −28.932 | 1.00 | 14.61 | | C |
| ATOM | 2457 | CB | ASP G | 385 | 2.452 | 27.379 | −30.366 | 1.00 | 14.71 | | C |
| ATOM | 2458 | CG | ASP G | 385 | 1.436 | 28.182 | −31.154 | 1.00 | 16.69 | | C |
| ATOM | 2459 | OD1 | ASP G | 385 | 0.270 | 28.259 | −30.713 | 1.00 | 18.63 | | O |
| ATOM | 2460 | OD2 | ASP G | 385 | 1.807 | 28.759 | −32.197 | 1.00 | 19.10 | | O |
| ATOM | 2461 | C | ASP G | 385 | 1.170 | 25.841 | −28.816 | 1.00 | 14.63 | | C |
| ATOM | 2462 | O | ASP G | 385 | −0.043 | 25.880 | −29.017 | 1.00 | 14.34 | | O |
| ATOM | 2463 | N | ILE G | 386 | 1.820 | 24.697 | −28.624 | 1.00 | 14.54 | | N |
| ATOM | 2464 | CA | ILE G | 386 | 1.116 | 23.413 | −28.688 | 1.00 | 14.46 | | C |
| ATOM | 2465 | CB | ILE G | 386 | −0.230 | 23.466 | −27.928 | 1.00 | 13.83 | | C |
| ATOM | 2466 | CG1 | ILE G | 386 | −0.004 | 23.458 | −26.415 | 1.00 | 13.44 | | C |
| ATOM | 2467 | CD1 | ILE G | 386 | −1.184 | 23.970 | −25.612 | 1.00 | 12.39 | | C |
| ATOM | 2468 | CG2 | ILE G | 386 | −1.119 | 22.301 | −28.341 | 1.00 | 13.51 | | C |
| ATOM | 2469 | C | ILE G | 386 | 0.791 | 23.079 | −30.134 | 1.00 | 14.73 | | C |
| ATOM | 2470 | O | ILE G | 386 | 1.282 | 22.092 | −30.683 | 1.00 | 14.68 | | O |
| ATOM | 2471 | N | PHE G | 387 | −0.191 | 23.797 | −30.667 | 1.00 | 14.71 | | N |
| ATOM | 2472 | CA | PHE G | 387 | −0.462 | 23.794 | −32.092 | 1.00 | 14.78 | | C |
| ATOM | 2473 | CB | PHE G | 387 | −1.889 | 24.254 | −32.366 | 1.00 | 14.77 | | C |
| ATOM | 2474 | CG | PHE G | 387 | −2.906 | 23.607 | −31.479 | 1.00 | 14.75 | | C |
| ATOM | 2475 | CD1 | PHE G | 387 | −3.418 | 24.287 | −30.390 | 1.00 | 15.03 | | C |
| ATOM | 2476 | CE1 | PHE G | 387 | −4.333 | 23.684 | −29.549 | 1.00 | 14.62 | | C |
| ATOM | 2477 | CZ | PHE G | 387 | −4.683 | 22.364 | −29.747 | 1.00 | 14.55 | | C |
| ATOM | 2478 | CE2 | PHE G | 387 | −4.113 | 21.651 | −30.780 | 1.00 | 14.68 | | C |
| ATOM | 2479 | CD2 | PHE G | 387 | −3.206 | 22.265 | −31.621 | 1.00 | 14.61 | | C |
| ATOM | 2480 | C | PHE G | 387 | 0.526 | 24.670 | −32.837 | 1.00 | 14.91 | | C |
| ATOM | 2481 | O | PHE G | 387 | 0.658 | 25.862 | −32.559 | 1.00 | 14.86 | | O |
| ATOM | 2482 | N | ASN G | 388 | 1.361 | 24.007 | −33.621 | 1.00 | 15.11 | | N |
| ATOM | 2483 | CA | ASN G | 388 | 2.352 | 24.663 | −34.443 | 1.00 | 15.38 | | C |
| ATOM | 2484 | CB | ASN G | 388 | 3.362 | 25.405 | −33.570 | 1.00 | 14.86 | | C |
| ATOM | 2485 | CG | ASN G | 388 | 4.097 | 24.480 | −32.625 | 1.00 | 14.86 | | C |
| ATOM | 2486 | OD1 | ASN G | 388 | 5.078 | 23.840 | −33.004 | 1.00 | 12.80 | | O |
| ATOM | 2487 | ND2 | ASN G | 388 | 3.575 | 24.338 | −31.413 | 1.00 | 15.97 | | N |
| ATOM | 2488 | C | ASN G | 388 | 3.057 | 23.576 | −35.229 | 1.00 | 16.11 | | C |
| ATOM | 2489 | O | ASN G | 388 | 3.416 | 22.537 | −34.670 | 1.00 | 15.92 | | O |
| ATOM | 2490 | N | PRO G | 389 | 3.071 | 23.722 | −36.557 | 1.00 | 16.99 | | N |
| ATOM | 2491 | CA | PRO G | 389 | 4.005 | 22.905 | −37.315 | 1.00 | 17.40 | | C |
| ATOM | 2492 | CB | PRO G | 389 | 4.169 | 23.689 | −38.618 | 1.00 | 17.72 | | C |
| ATOM | 2493 | CG | PRO G | 389 | 3.905 | 25.099 | −38.236 | 1.00 | 17.47 | | C |
| ATOM | 2494 | CD | PRO G | 389 | 2.825 | 25.023 | −37.203 | 1.00 | 17.05 | | C |
| ATOM | 2495 | C | PRO G | 389 | 5.338 | 22.827 | −36.583 | 1.00 | 17.86 | | C |
| ATOM | 2496 | O | PRO G | 389 | 5.638 | 23.687 | −35.755 | 1.00 | 18.45 | | O |
| ATOM | 2497 | N | LYS G | 390 | 6.139 | 21.817 | −36.902 | 1.00 | 17.97 | | N |
| ATOM | 2498 | CA | LYS G | 390 | 7.443 | 21.658 | −36.273 | 1.00 | 18.06 | | C |
| ATOM | 2499 | CB | LYS G | 390 | 7.947 | 22.997 | −35.730 | 1.00 | 17.92 | | C |
| ATOM | 2500 | CG | LYS G | 390 | 8.466 | 23.945 | −36.798 | 1.00 | 18.58 | | C |
| ATOM | 2501 | CD | LYS G | 390 | 9.962 | 23.777 | −37.006 | 1.00 | 20.61 | | C |
| ATOM | 2502 | CE | LYS G | 390 | 10.562 | 24.985 | −37.707 | 1.00 | 21.48 | | C |
| ATOM | 2503 | NZ | LYS G | 390 | 10.191 | 25.032 | −39.148 | 1.00 | 23.16 | | N |
| ATOM | 2504 | C | LYS G | 390 | 7.398 | 20.623 | −35.156 | 1.00 | 18.01 | | C |
| ATOM | 2505 | O | LYS G | 390 | 8.177 | 19.670 | −35.152 | 1.00 | 18.43 | | O |
| ATOM | 2506 | N | TYR G | 391 | 6.481 | 20.805 | −34.213 | 1.00 | 17.68 | | N |
| ATOM | 2507 | CA | TYR G | 391 | 6.234 | 19.780 | −33.211 | 1.00 | 17.33 | | C |
| ATOM | 2508 | CB | TYR G | 391 | 6.862 | 20.158 | −31.872 | 1.00 | 17.42 | | C |
| ATOM | 2509 | CG | TYR G | 391 | 7.485 | 18.979 | −31.173 | 1.00 | 17.27 | | C |
| ATOM | 2510 | CD1 | TYR G | 391 | 8.188 | 18.028 | −31.898 | 1.00 | 17.29 | | C |
| ATOM | 2511 | CE1 | TYR G | 391 | 8.672 | 16.885 | −31.297 | 1.00 | 17.32 | | C |
| ATOM | 2512 | CZ | TYR G | 391 | 8.408 | 16.653 | −29.965 | 1.00 | 16.51 | | C |
| ATOM | 2513 | OH | TYR G | 391 | 8.940 | 15.541 | −29.354 | 1.00 | 16.65 | | O |
| ATOM | 2514 | CE2 | TYR G | 391 | 7.656 | 17.552 | −29.237 | 1.00 | 16.52 | | C |
| ATOM | 2515 | CD2 | TYR G | 391 | 7.167 | 18.687 | −29.853 | 1.00 | 17.24 | | C |
| ATOM | 2516 | C | TYR G | 391 | 4.757 | 19.471 | −33.034 | 1.00 | 17.43 | | C |
| ATOM | 2517 | O | TYR G | 391 | 3.984 | 20.332 | −32.614 | 1.00 | 17.70 | | O |
| ATOM | 2518 | N | ASP G | 392 | 4.398 | 18.202 | −33.196 | 1.00 | 17.56 | | N |
| ATOM | 2519 | CA | ASP G | 392 | 3.191 | 17.693 | −32.565 | 1.00 | 17.69 | | C |
| ATOM | 2520 | CB | ASP G | 392 | 2.610 | 16.498 | −33.322 | 1.00 | 17.89 | | C |
| ATOM | 2521 | CG | ASP G | 392 | 3.494 | 16.038 | −34.456 | 1.00 | 18.86 | | C |
| ATOM | 2522 | OD1 | ASP G | 392 | 4.636 | 15.613 | −34.185 | 1.00 | 20.34 | | O |
| ATOM | 2523 | OD2 | ASP G | 392 | 3.008 | 15.998 | −35.604 | 1.00 | 18.51 | | O |
| ATOM | 2524 | C | ASP G | 392 | 3.428 | 17.344 | −31.107 | 1.00 | 17.11 | | C |
| ATOM | 2525 | O | ASP G | 392 | 4.495 | 16.860 | −30.731 | 1.00 | 17.04 | | O |
| ATOM | 2526 | N | CYS G | 393 | 2.452 | 17.684 | −30.280 | 1.00 | 16.33 | | N |
| ATOM | 2527 | CA | CYS G | 393 | 2.723 | 18.199 | −28.957 | 1.00 | 16.17 | | C |
| ATOM | 2528 | CB | CYS G | 393 | 2.419 | 19.693 | −28.910 | 1.00 | 16.11 | | C |
| ATOM | 2529 | SG | CYS G | 393 | 3.237 | 20.556 | −27.570 | 1.00 | 20.23 | | S |
| ATOM | 2530 | C | CYS G | 393 | 1.812 | 17.471 | −27.995 | 1.00 | 15.37 | | C |
| ATOM | 2531 | O | CYS G | 393 | 0.592 | 17.525 | −28.139 | 1.00 | 15.15 | | O |
| ATOM | 2532 | N | LYS G | 394 | 2.399 | 16.618 | −27.168 | 1.00 | 14.91 | | N |
| ATOM | 2533 | CA | LYS G | 394 | 1.608 | 15.678 | −26.398 | 1.00 | 14.50 | | C |
| ATOM | 2534 | CB | LYS G | 394 | 2.495 | 14.607 | −25.771 | 1.00 | 14.60 | | C |
| ATOM | 2535 | CG | LYS G | 394 | 3.531 | 14.052 | −26.730 | 1.00 | 16.44 | | C |
| ATOM | 2536 | CD | LYS G | 394 | 4.034 | 12.699 | −26.272 | 1.00 | 20.45 | | C |
| ATOM | 2537 | CE | LYS G | 394 | 4.868 | 12.823 | −25.009 | 1.00 | 23.05 | | C |
| ATOM | 2538 | NZ | LYS G | 394 | 4.057 | 13.264 | −23.841 | 1.00 | 24.13 | | N |
| ATOM | 2539 | C | LYS G | 394 | 0.767 | 16.388 | −25.344 | 1.00 | 14.00 | | C |
| ATOM | 2540 | O | LYS G | 394 | 1.249 | 17.268 | −24.630 | 1.00 | 14.14 | | O |
| ATOM | 2541 | N | ILE G | 395 | −0.486 | 15.955 | −25.236 | 1.00 | 13.28 | | N |
| ATOM | 2542 | CA | ILE G | 395 | −1.587 | 16.768 | −24.725 | 1.00 | 12.89 | | C |
| ATOM | 2543 | CB | ILE G | 395 | −2.234 | 17.626 | −25.843 | 1.00 | 12.75 | | C |
| ATOM | 2544 | CG1 | ILE G | 395 | −1.418 | 18.897 | −26.097 | 1.00 | 13.58 | | C |
| ATOM | 2545 | CD1 | ILE G | 395 | −1.635 | 19.507 | −27.466 | 1.00 | 15.33 | | C |
| ATOM | 2546 | CG2 | ILE G | 395 | −3.669 | 17.980 | −25.477 | 1.00 | 12.16 | | C |
| ATOM | 2547 | C | ILE G | 395 | −2.631 | 15.761 | −24.284 | 1.00 | 13.15 | | C |
| ATOM | 2548 | O | ILE G | 395 | −2.826 | 14.746 | −24.951 | 1.00 | 13.45 | | O |
| ATOM | 2549 | N | MET G | 396 | −3.440 | 16.127 | −23.303 | 1.00 | 13.06 | | N |
| ATOM | 2550 | CA | MET G | 396 | −4.668 | 15.392 | −23.085 | 1.00 | 13.00 | | C |
| ATOM | 2551 | CB | MET G | 396 | −4.579 | 14.539 | −21.824 | 1.00 | 13.34 | | C |
| ATOM | 2552 | CG | MET G | 396 | −5.208 | 15.167 | −20.600 | 1.00 | 14.30 | | C |
| ATOM | 2553 | SD | MET G | 396 | −5.006 | 14.087 | −19.179 | 1.00 | 18.50 | | S |
| ATOM | 2554 | CE | MET G | 396 | −3.465 | 13.282 | −19.611 | 1.00 | 17.80 | | C |
| ATOM | 2555 | C | MET G | 396 | −5.889 | 16.288 | −23.050 | 1.00 | 12.96 | | C |
| ATOM | 2556 | O | MET G | 396 | −5.811 | 17.453 | −22.663 | 1.00 | 13.04 | | O |
| ATOM | 2557 | N | THR G | 397 | −7.037 | 15.686 | −23.336 | 1.00 | 12.78 | | N |
| ATOM | 2558 | CA | THR G | 397 | −8.200 | 16.415 | −23.815 | 1.00 | 12.51 | | C |
| ATOM | 2559 | CB | THR G | 397 | −8.653 | 15.898 | −25.193 | 1.00 | 12.39 | | C |
| ATOM | 2560 | OG1 | THR G | 397 | −7.837 | 16.481 | −26.216 | 1.00 | 12.50 | | O |
| ATOM | 2561 | CG2 | THR G | 397 | −10.114 | 16.250 | −25.447 | 1.00 | 12.55 | | C |
| ATOM | 2562 | C | THR G | 397 | −9.343 | 16.223 | −22.834 | 1.00 | 12.58 | | C |
| ATOM | 2563 | O | THR G | 397 | −9.563 | 15.117 | −22.340 | 1.00 | 12.88 | | O |
| ATOM | 2564 | N | SER G | 398 | −10.157 | 17.255 | −22.663 | 1.00 | 12.55 | | N |
| ATOM | 2565 | CA | SER G | 398 | −11.452 | 17.049 | −22.049 | 1.00 | 12.76 | | C |
| ATOM | 2566 | CB | SER G | 398 | −11.323 | 16.949 | −20.527 | 1.00 | 12.52 | | C |
| ATOM | 2567 | OG | SER G | 398 | −12.592 | 16.781 | −19.919 | 1.00 | 12.72 | | O |
| ATOM | 2568 | C | SER G | 398 | −12.478 | 18.103 | −22.410 | 1.00 | 12.99 | | C |
| ATOM | 2569 | O | SER G | 398 | −12.269 | 18.917 | −23.311 | 1.00 | 13.13 | | O |
| ATOM | 2570 | N | LYS G | 399 | −13.435 | 18.245 | −21.504 | 1.00 | 13.01 | | N |
| ATOM | 2571 | CA | LYS G | 399 | −14.399 | 19.325 | −21.544 | 1.00 | 13.29 | | C |
| ATOM | 2572 | CB | LYS G | 399 | −15.777 | 18.777 | −21.908 | 1.00 | 13.79 | | C |
| ATOM | 2573 | CG | LYS G | 399 | −16.044 | 18.714 | −23.399 | 1.00 | 14.34 | | C |
| ATOM | 2574 | CD | LYS G | 399 | −16.965 | 19.840 | −23.833 | 1.00 | 16.43 | | C |
| ATOM | 2575 | CE | LYS G | 399 | −17.285 | 19.750 | −25.315 | 1.00 | 17.37 | | C |
| ATOM | 2576 | NZ | LYS G | 399 | −17.904 | 21.003 | −25.828 | 1.00 | 16.62 | | N |
| ATOM | 2577 | C | LYS G | 399 | −14.460 | 19.996 | −20.179 | 1.00 | 13.09 | | C |
| ATOM | 2578 | O | LYS G | 399 | −14.619 | 21.213 | −20.084 | 1.00 | 12.87 | | O |
| ATOM | 2579 | N | THR G | 400 | −14.348 | 19.198 | −19.123 | 1.00 | 13.04 | | N |
| ATOM | 2580 | CA | THR G | 400 | −14.535 | 19.715 | −17.776 | 1.00 | 13.38 | | C |
| ATOM | 2581 | CB | THR G | 400 | −15.257 | 18.709 | −16.861 | 1.00 | 13.74 | | C |
| ATOM | 2582 | OG1 | THR G | 400 | −15.856 | 17.678 | −17.655 | 1.00 | 14.38 | | O |
| ATOM | 2583 | CG2 | THR G | 400 | −16.337 | 19.409 | −16.051 | 1.00 | 14.32 | | C |
| ATOM | 2584 | C | THR G | 400 | −13.226 | 20.134 | −17.120 | 1.00 | 13.20 | | C |
| ATOM | 2585 | O | THR G | 400 | −12.169 | 19.594 | −17.445 | 1.00 | 13.12 | | O |
| ATOM | 2586 | N | ASP G | 401 | −13.378 | 20.770 | −15.963 | 1.00 | 12.97 | | N |
| ATOM | 2587 | CA | ASP G | 401 | −12.358 | 21.654 | −15.396 | 1.00 | 12.50 | | C |
| ATOM | 2588 | CB | ASP G | 401 | −13.014 | 22.766 | −14.590 | 1.00 | 12.73 | | C |
| ATOM | 2589 | CG | ASP G | 401 | −12.765 | 24.122 | −15.183 | 1.00 | 13.98 | | C |
| ATOM | 2590 | OD1 | ASP G | 401 | −13.073 | 24.301 | −16.380 | 1.00 | 16.55 | | O |
| ATOM | 2591 | OD2 | ASP G | 401 | −12.168 | 24.973 | −14.492 | 1.00 | 14.80 | | O |
| ATOM | 2592 | C | ASP G | 401 | −11.269 | 21.029 | −14.529 | 1.00 | 12.13 | | C |
| ATOM | 2593 | O | ASP G | 401 | −11.547 | 20.130 | −13.729 | 1.00 | 12.31 | | O |
| ATOM | 2594 | N | VAL G | 402 | −10.200 | 21.822 | −14.390 | 1.00 | 11.49 | | N |
| ATOM | 2595 | CA | VAL G | 402 | −9.667 | 22.261 | −13.080 | 1.00 | 10.74 | | C |
| ATOM | 2596 | CB | VAL G | 402 | −8.369 | 21.519 | −12.744 | 1.00 | 10.60 | | C |
| ATOM | 2597 | CG1 | VAL G | 402 | −7.449 | 22.413 | −11.914 | 1.00 | 11.26 | | C |
| ATOM | 2598 | CG2 | VAL G | 402 | −8.672 | 20.211 | −12.019 | 1.00 | 10.99 | | C |
| ATOM | 2599 | C | VAL G | 402 | −9.360 | 23.769 | −12.883 | 1.00 | 10.24 | | C |
| ATOM | 2600 | O | VAL G | 402 | −9.387 | 24.539 | −13.845 | 1.00 | 10.30 | | O |
| ATOM | 2601 | N | SER G | 403 | −8.762 | 24.081 | −11.719 | 1.00 | 9.76 | | N |
| ATOM | 2602 | CA | SER G | 403 | −8.493 | 25.474 | −11.271 | 1.00 | 9.18 | | C |
| ATOM | 2603 | CB | SER G | 403 | −9.790 | 26.197 | −10.893 | 1.00 | 9.41 | | C |
| ATOM | 2604 | OG | SER G | 403 | −10.714 | 25.319 | −10.274 | 1.00 | 10.82 | | O |
| ATOM | 2605 | C | SER G | 403 | −7.394 | 25.745 | −10.199 | 1.00 | 8.51 | | C |
| ATOM | 2606 | O | SER G | 403 | −7.465 | 25.236 | −9.076 | 1.00 | 8.30 | | O |
| ATOM | 2607 | N | SER G | 404 | −6.524 | 26.720 | −10.502 | 1.00 | 8.35 | | N |
| ATOM | 2608 | CA | SER G | 404 | −5.785 | 27.535 | −9.507 | 1.00 | 8.18 | | C |
| ATOM | 2609 | CB | SER G | 404 | −4.851 | 26.657 | −8.669 | 1.00 | 8.18 | | C |
| ATOM | 2610 | OG | SER G | 404 | −3.507 | 26.782 | −9.100 | 1.00 | 8.83 | | O |
| ATOM | 2611 | C | SER G | 404 | −4.979 | 28.665 | −10.187 | 1.00 | 7.62 | | C |
| ATOM | 2612 | O | SER G | 404 | −5.183 | 28.942 | −11.369 | 1.00 | 7.57 | | O |
| ATOM | 2613 | N | SER G | 405 | −4.041 | 29.282 | −9.462 | 1.00 | 7.08 | | N |
| ATOM | 2614 | CA | SER G | 405 | −3.258 | 30.409 | −10.003 | 1.00 | 6.53 | | C |
| ATOM | 2615 | CB | SER G | 405 | −3.844 | 31.752 | −9.555 | 1.00 | 6.42 | | C |
| ATOM | 2616 | OG | SER G | 405 | −2.883 | 32.790 | −9.662 | 1.00 | 5.66 | | O |
| ATOM | 2617 | C | SER G | 405 | −1.756 | 30.364 | −9.693 | 1.00 | 6.28 | | C |
| ATOM | 2618 | O | SER G | 405 | −1.338 | 29.830 | −8.666 | 1.00 | 5.98 | | O |
| ATOM | 2619 | N | VAL G | 406 | −0.980 | 31.079 | −10.506 | 1.00 | 6.22 | | N |
| ATOM | 2620 | CA | VAL G | 406 | 0.479 | 31.053 | −10.436 | 1.00 | 6.50 | | C |
| ATOM | 2621 | CB | VAL G | 406 | 1.068 | 30.055 | −11.453 | 1.00 | 6.45 | | C |
| ATOM | 2622 | CG1 | VAL G | 406 | 2.518 | 29.743 | −11.116 | 1.00 | 7.66 | | C |
| ATOM | 2623 | CG2 | VAL G | 406 | 0.236 | 28.783 | −11.495 | 1.00 | 7.38 | | C |
| ATOM | 2624 | C | VAL G | 406 | 1.029 | 32.438 | −10.760 | 1.00 | 6.24 | | C |
| ATOM | 2625 | O | VAL G | 406 | 0.763 | 32.981 | −11.832 | 1.00 | 6.14 | | O |
| ATOM | 2626 | N | ILE G | 407 | 1.803 | 33.001 | −9.840 | 1.00 | 6.32 | | N |
| ATOM | 2627 | CA | ILE G | 407 | 2.315 | 34.354 | −10.014 | 1.00 | 6.60 | | C |
| ATOM | 2628 | CB | ILE G | 407 | 2.445 | 35.097 | −8.674 | 1.00 | 6.27 | | C |
| ATOM | 2629 | CG1 | ILE G | 407 | 1.070 | 35.564 | −8.194 | 1.00 | 6.19 | | C |
| ATOM | 2630 | CD1 | ILE G | 407 | 1.086 | 36.200 | −6.824 | 1.00 | 8.34 | | C |
| ATOM | 2631 | CG2 | ILE G | 407 | 3.391 | 36.282 | −8.813 | 1.00 | 6.42 | | C |
| ATOM | 2632 | C | ILE G | 407 | 3.657 | 34.361 | −10.728 | 1.00 | 7.20 | | C |
| ATOM | 2633 | O | ILE G | 407 | 4.576 | 33.631 | −10.356 | 1.00 | 7.42 | | O |
| ATOM | 2634 | N | THR G | 408 | 3.797 | 35.273 | −11.682 | 1.00 | 7.57 | | N |
| ATOM | 2635 | CA | THR G | 408 | 4.983 | 35.324 | −12.517 | 1.00 | 8.19 | | C |
| ATOM | 2636 | CB | THR G | 408 | 4.622 | 35.305 | −14.011 | 1.00 | 8.15 | | C |
| ATOM | 2637 | OG1 | THR G | 408 | 3.785 | 36.426 | −14.318 | 1.00 | 8.88 | | O |
| ATOM | 2638 | CG2 | THR G | 408 | 3.891 | 34.020 | −14.364 | 1.00 | 7.93 | | C |
| ATOM | 2639 | C | THR G | 408 | 5.804 | 36.569 | −12.225 | 1.00 | 8.55 | | C |
| ATOM | 2640 | O | THR G | 408 | 5.418 | 37.408 | −11.410 | 1.00 | 9.07 | | O |
| ATOM | 2641 | N | SER G | 409 | 6.918 | 36.698 | −12.935 | 1.00 | 8.70 | | N |
| ATOM | 2642 | CA | SER G | 409 | 7.843 | 37.799 | −12.723 | 1.00 | 9.20 | | C |
| ATOM | 2643 | CB | SER G | 409 | 9.067 | 37.643 | −13.628 | 1.00 | 9.72 | | C |
| ATOM | 2644 | OG | SER G | 409 | 9.571 | 36.320 | −13.581 | 1.00 | 10.75 | | O |
| ATOM | 2645 | C | SER G | 409 | 7.153 | 39.125 | −13.007 | 1.00 | 9.01 | | C |
| ATOM | 2646 | O | SER G | 409 | 7.433 | 40.135 | −12.359 | 1.00 | 9.02 | | O |
| ATOM | 2647 | N | LEU G | 410 | 6.259 | 39.118 | −13.990 | 1.00 | 8.91 | | N |
| ATOM | 2648 | CA | LEU G | 410 | 5.790 | 40.352 | −14.604 | 1.00 | 9.29 | | C |
| ATOM | 2649 | CB | LEU G | 410 | 6.438 | 40.546 | −15.976 | 1.00 | 9.61 | | C |
| ATOM | 2650 | CG | LEU G | 410 | 7.245 | 41.834 | −16.150 | 1.00 | 9.70 | | C |
| ATOM | 2651 | CD1 | LEU G | 410 | 8.433 | 41.858 | −15.200 | 1.00 | 11.11 | | C |
| ATOM | 2652 | CD2 | LEU G | 410 | 7.705 | 41.994 | −17.591 | 1.00 | 9.16 | | C |
| ATOM | 2653 | C | LEU G | 410 | 4.272 | 40.351 | −14.729 | 1.00 | 9.12 | | C |
| ATOM | 2654 | O | LEU G | 410 | 3.683 | 41.248 | −15.332 | 1.00 | 9.08 | | O |
| ATOM | 2655 | N | GLY G | 411 | 3.646 | 39.332 | −14.154 | 1.00 | 9.03 | | N |
| ATOM | 2656 | CA | GLY G | 411 | 2.203 | 39.315 | −13.982 | 1.00 | 8.44 | | C |
| ATOM | 2657 | C | GLY G | 411 | 1.765 | 38.048 | −13.284 | 1.00 | 7.91 | | C |
| ATOM | 2658 | O | GLY G | 411 | 2.435 | 37.568 | −12.369 | 1.00 | 7.85 | | O |
| ATOM | 2659 | N | ALA G | 412 | 0.674 | 37.465 | −13.763 | 1.00 | 7.30 | | N |
| ATOM | 2660 | CA | ALA G | 412 | 0.129 | 36.264 | −13.153 | 1.00 | 6.92 | | C |
| ATOM | 2661 | CB | ALA G | 412 | −0.742 | 36.623 | −11.960 | 1.00 | 6.90 | | C |
| ATOM | 2662 | C | ALA G | 412 | −0.661 | 35.453 | −14.166 | 1.00 | 6.99 | | C |
| ATOM | 2663 | O | ALA G | 412 | −1.257 | 36.002 | −15.093 | 1.00 | 6.94 | | O |
| ATOM | 2664 | N | ILE G | 413 | −0.621 | 34.137 | −14.006 | 1.00 | 7.29 | | N |
| ATOM | 2665 | CA | ILE G | 413 | −1.478 | 33.244 | −14.766 | 1.00 | 7.49 | | C |
| ATOM | 2666 | CB | ILE G | 413 | −0.711 | 31.990 | −15.201 | 1.00 | 7.24 | | C |
| ATOM | 2667 | CG1 | ILE G | 413 | 0.314 | 32.350 | −16.278 | 1.00 | 7.18 | | C |
| ATOM | 2668 | CD1 | ILE G | 413 | 0.927 | 31.150 | −16.973 | 1.00 | 7.70 | | C |
| ATOM | 2669 | CG2 | ILE G | 413 | −1.674 | 30.916 | −15.688 | 1.00 | 7.70 | | C |
| ATOM | 2670 | C | ILE G | 413 | −2.657 | 32.822 | −13.908 | 1.00 | 7.94 | | C |
| ATOM | 2671 | O | ILE G | 413 | −2.544 | 32.749 | −12.685 | 1.00 | 8.39 | | O |
| ATOM | 2672 | N | VAL G | 414 | −3.782 | 32.521 | −14.544 | 1.00 | 8.22 | | N |
| ATOM | 2673 | CA | VAL G | 414 | −4.909 | 31.961 | −13.816 | 1.00 | 8.77 | | C |
| ATOM | 2674 | CB | VAL G | 414 | −5.865 | 33.050 | −13.296 | 1.00 | 8.57 | | C |
| ATOM | 2675 | CG1 | VAL G | 414 | −6.133 | 34.085 | −14.376 | 1.00 | 9.12 | | C |
| ATOM | 2676 | CG2 | VAL G | 414 | −7.162 | 32.427 | −12.803 | 1.00 | 8.90 | | C |
| ATOM | 2677 | C | VAL G | 414 | −5.674 | 30.901 | −14.594 | 1.00 | 8.92 | | C |
| ATOM | 2678 | O | VAL G | 414 | −6.114 | 31.129 | −15.722 | 1.00 | 9.00 | | O |
| ATOM | 2679 | N | SER G | 415 | −5.750 | 29.714 | −14.004 | 1.00 | 8.97 | | N |
| ATOM | 2680 | CA | SER G | 415 | −6.472 | 28.598 | −14.592 | 1.00 | 9.02 | | C |
| ATOM | 2681 | CB | SER G | 415 | −5.625 | 27.328 | −14.520 | 1.00 | 8.98 | | C |
| ATOM | 2682 | OG | SER G | 415 | −4.353 | 27.532 | −15.108 | 1.00 | 9.54 | | O |
| ATOM | 2683 | C | SER G | 415 | −7.791 | 28.390 | −13.864 | 1.00 | 9.36 | | C |
| ATOM | 2684 | O | SER G | 415 | −7.818 | 27.884 | −12.742 | 1.00 | 9.61 | | O |
| ATOM | 2685 | N | CYS G | 416 | −8.854 | 28.950 | −14.430 | 1.00 | 9.57 | | N |
| ATOM | 2686 | CA | CYS G | 416 | −10.175 | 28.857 | −13.831 | 1.00 | 9.61 | | C |
| ATOM | 2687 | CB | CYS G | 416 | −10.895 | 30.201 | −13.912 | 1.00 | 9.62 | | C |
| ATOM | 2688 | SG | CYS G | 416 | −12.025 | 30.497 | −12.543 | 1.00 | 11.33 | | S |
| ATOM | 2689 | C | CYS G | 416 | −11.000 | 27.796 | −14.534 | 1.00 | 9.61 | | C |
| ATOM | 2690 | O | CYS G | 416 | −11.361 | 27.950 | −15.700 | 1.00 | 9.80 | | O |
| ATOM | 2691 | N | TYR G | 417 | −11.298 | 26.717 | −13.821 | 1.00 | 9.48 | | N |
| ATOM | 2692 | CA | TYR G | 417 | −11.930 | 25.566 | −14.438 | 1.00 | 9.28 | | C |
| ATOM | 2693 | CB | TYR G | 417 | −10.888 | 24.501 | −14.782 | 1.00 | 9.17 | | C |
| ATOM | 2694 | CG | TYR G | 417 | −10.011 | 24.860 | −15.962 | 1.00 | 8.71 | | C |
| ATOM | 2695 | CD1 | TYR G | 417 | −8.661 | 24.543 | −15.968 | 1.00 | 8.54 | | C |
| ATOM | 2696 | CE1 | TYR G | 417 | −7.857 | 24.869 | −17.043 | 1.00 | 9.56 | | C |
| ATOM | 2697 | CZ | TYR G | 417 | −8.407 | 25.480 | −18.149 | 1.00 | 9.83 | | C |
| ATOM | 2698 | OH | TYR G | 417 | −7.602 | 25.828 | −19.207 | 1.00 | 10.51 | | O |
| ATOM | 2699 | CE2 | TYR G | 417 | −9.738 | 25.830 | −18.159 | 1.00 | 9.36 | | C |
| ATOM | 2700 | CD2 | TYR G | 417 | −10.532 | 25.522 | −17.068 | 1.00 | 8.95 | | C |
| ATOM | 2701 | C | TYR G | 417 | −13.034 | 24.978 | −13.566 | 1.00 | 9.35 | | C |
| ATOM | 2702 | O | TYR G | 417 | −13.171 | 25.332 | −12.395 | 1.00 | 9.11 | | O |
| ATOM | 2703 | N | GLY G | 418 | −13.848 | 24.115 | −14.165 | 1.00 | 9.71 | | N |
| ATOM | 2704 | CA | GLY G | 418 | −14.993 | 23.534 | −13.481 | 1.00 | 9.95 | | C |
| ATOM | 2705 | C | GLY G | 418 | −15.925 | 24.594 | −12.936 | 1.00 | 10.09 | | C |
| ATOM | 2706 | O | GLY G | 418 | −16.170 | 25.611 | −13.585 | 1.00 | 10.17 | | O |
| ATOM | 2707 | N | LYS G | 419 | −16.323 | 24.426 | −11.681 | 1.00 | 10.17 | | N |
| ATOM | 2708 | CA | LYS G | 419 | −17.328 | 25.289 | −11.075 | 1.00 | 10.50 | | C |
| ATOM | 2709 | CB | LYS G | 419 | −18.123 | 24.523 | −10.015 | 1.00 | 11.09 | | C |
| ATOM | 2710 | CG | LYS G | 419 | −19.389 | 23.867 | −10.538 | 1.00 | 12.94 | | C |
| ATOM | 2711 | CD | LYS G | 419 | −20.304 | 23.450 | −9.397 | 1.00 | 15.52 | | C |
| ATOM | 2712 | CE | LYS G | 419 | −21.702 | 23.127 | −9.899 | 1.00 | 17.07 | | C |
| ATOM | 2713 | NZ | LYS G | 419 | −21.688 | 22.058 | −10.936 | 1.00 | 19.30 | | N |
| ATOM | 2714 | C | LYS G | 419 | −16.737 | 26.560 | −10.464 | 1.00 | 10.23 | | C |
| ATOM | 2715 | O | LYS G | 419 | −17.451 | 27.325 | −9.817 | 1.00 | 10.58 | | O |
| ATOM | 2716 | N | THR G | 420 | −15.429 | 26.755 | −10.607 | 1.00 | 10.10 | | N |
| ATOM | 2717 | CA | THR G | 420 | −14.730 | 27.754 | −9.801 | 1.00 | 9.74 | | C |
| ATOM | 2718 | CB | THR G | 420 | −13.232 | 27.432 | −9.651 | 1.00 | 9.28 | | C |
| ATOM | 2719 | OG1 | THR G | 420 | −12.992 | 26.073 | −10.034 | 1.00 | 9.21 | | O |
| ATOM | 2720 | CG2 | THR G | 420 | −12.788 | 27.635 | −8.209 | 1.00 | 9.03 | | C |
| ATOM | 2721 | C | THR G | 420 | −14.900 | 29.178 | −10.329 | 1.00 | 9.95 | | C |
| ATOM | 2722 | O | THR G | 420 | −15.143 | 29.387 | −11.518 | 1.00 | 10.35 | | O |
| ATOM | 2723 | N | LYS G | 421 | −14.816 | 30.149 | −9.424 | 1.00 | 10.07 | | N |
| ATOM | 2724 | CA | LYS G | 421 | −14.950 | 31.559 | −9.780 | 1.00 | 10.21 | | C |
| ATOM | 2725 | CB | LYS G | 421 | −15.973 | 32.239 | −8.867 | 1.00 | 10.30 | | C |
| ATOM | 2726 | CG | LYS G | 421 | −17.368 | 32.341 | −9.457 | 1.00 | 12.43 | | C |
| ATOM | 2727 | CD | LYS G | 421 | −18.345 | 32.931 | −8.452 | 1.00 | 15.12 | | C |
| ATOM | 2728 | CE | LYS G | 421 | −19.766 | 32.470 | −8.727 | 1.00 | 15.21 | | C |
| ATOM | 2729 | NZ | LYS G | 421 | −20.617 | 32.544 | −7.508 | 1.00 | 15.98 | | N |
| ATOM | 2730 | C | LYS G | 421 | −13.608 | 32.271 | −9.658 | 1.00 | 10.09 | | C |
| ATOM | 2731 | O | LYS G | 421 | −12.911 | 32.125 | −8.653 | 1.00 | 10.36 | | O |
| ATOM | 2732 | N | CYS G | 422 | −13.271 | 33.085 | −10.653 | 1.00 | 9.81 | | N |
| ATOM | 2733 | CA | CYS G | 422 | −12.006 | 33.808 | −10.631 | 1.00 | 9.56 | | C |
| ATOM | 2734 | CB | CYS G | 422 | −10.953 | 33.092 | −11.475 | 1.00 | 9.72 | | C |
| ATOM | 2735 | SG | CYS G | 422 | −10.831 | 31.329 | −11.131 | 1.00 | 11.42 | | S |
| ATOM | 2736 | C | CYS G | 422 | −12.126 | 35.268 | −11.044 | 1.00 | 9.18 | | C |
| ATOM | 2737 | O | CYS G | 422 | −12.927 | 35.624 | −11.909 | 1.00 | 9.27 | | O |
| ATOM | 2738 | N | THR G | 423 | −11.232 | 36.084 | −10.497 | 1.00 | 8.93 | | N |
| ATOM | 2739 | CA | THR G | 423 | −11.422 | 37.525 | −10.427 | 1.00 | 8.64 | | C |
| ATOM | 2740 | CB | THR G | 423 | −12.062 | 37.938 | −9.088 | 1.00 | 8.32 | | C |
| ATOM | 2741 | OG1 | THR G | 423 | −13.201 | 37.111 | −8.821 | 1.00 | 7.97 | | O |
| ATOM | 2742 | CG2 | THR G | 423 | −12.494 | 39.396 | −9.130 | 1.00 | 8.36 | | C |
| ATOM | 2743 | C | THR G | 423 | −10.059 | 38.190 | −10.513 | 1.00 | 8.86 | | C |
| ATOM | 2744 | O | THR G | 423 | −9.183 | 37.928 | −9.688 | 1.00 | 9.26 | | O |
| ATOM | 2745 | N | ALA G | 424 | −9.913 | 39.124 | −11.444 | 1.00 | 8.74 | | N |
| ATOM | 2746 | CA | ALA G | 424 | −8.792 | 40.052 | −11.408 | 1.00 | 8.79 | | C |
| ATOM | 2747 | CB | ALA G | 424 | −8.164 | 40.180 | −12.786 | 1.00 | 8.94 | | C |
| ATOM | 2748 | C | ALA G | 424 | −9.240 | 41.414 | −10.894 | 1.00 | 8.84 | | C |
| ATOM | 2749 | O | ALA G | 424 | −10.084 | 42.070 | −11.504 | 1.00 | 9.24 | | O |
| ATOM | 2750 | N | SER G | 425 | −8.669 | 41.836 | −9.771 | 1.00 | 8.81 | | N |
| ATOM | 2751 | CA | SER G | 425 | −9.007 | 43.124 | −9.180 | 1.00 | 8.85 | | C |
| ATOM | 2752 | CB | SER G | 425 | −9.483 | 42.942 | −7.737 | 1.00 | 8.94 | | C |
| ATOM | 2753 | OG | SER G | 425 | −9.918 | 41.613 | −7.507 | 1.00 | 7.69 | | O |
| ATOM | 2754 | C | SER G | 425 | −7.815 | 44.072 | −9.222 | 1.00 | 9.01 | | C |
| ATOM | 2755 | O | SER G | 425 | −6.669 | 43.651 | −9.059 | 1.00 | 8.63 | | O |
| ATOM | 2756 | N | ASN G | 426 | −8.089 | 45.354 | −9.441 | 1.00 | 9.54 | | N |
| ATOM | 2757 | CA | ASN G | 426 | −7.033 | 46.328 | −9.686 | 1.00 | 10.19 | | C |
| ATOM | 2758 | CB | ASN G | 426 | −7.537 | 47.462 | −10.583 | 1.00 | 9.77 | | C |
| ATOM | 2759 | CG | ASN G | 426 | −8.273 | 48.540 | −9.808 | 1.00 | 9.43 | | C |
| ATOM | 2760 | OD1 | ASN G | 426 | −8.253 | 48.561 | −8.578 | 1.00 | 9.01 | | O |
| ATOM | 2761 | ND2 | ASN G | 426 | −8.938 | 49.436 | −10.528 | 1.00 | 9.01 | | N |
| ATOM | 2762 | C | ASN G | 426 | −6.434 | 46.891 | −8.400 | 1.00 | 11.16 | | C |
| ATOM | 2763 | O | ASN G | 426 | −6.629 | 46.340 | −7.316 | 1.00 | 11.06 | | O |
| ATOM | 2764 | N | LYS G | 427 | −5.658 | 47.961 | −8.543 | 1.00 | 12.38 | | N |
| ATOM | 2765 | CA | LYS G | 427 | −5.016 | 48.627 | −7.413 | 1.00 | 13.52 | | C |
| ATOM | 2766 | CB | LYS G | 427 | −4.455 | 49.981 | −7.851 | 1.00 | 14.00 | | C |
| ATOM | 2767 | CG | LYS G | 427 | −2.987 | 49.956 | −8.233 | 1.00 | 16.18 | | C |
| ATOM | 2768 | CD | LYS G | 427 | −2.434 | 51.366 | −8.365 | 1.00 | 18.24 | | C |
| ATOM | 2769 | CE | LYS G | 427 | −0.955 | 51.413 | −8.022 | 1.00 | 19.72 | | C |
| ATOM | 2770 | NZ | LYS G | 427 | −0.707 | 51.116 | −6.584 | 1.00 | 21.43 | | N |
| ATOM | 2771 | C | LYS G | 427 | −5.976 | 48.829 | −6.245 | 1.00 | 13.71 | | C |
| ATOM | 2772 | O | LYS G | 427 | −5.630 | 48.562 | −5.093 | 1.00 | 13.63 | | O |
| ATOM | 2773 | N | ASN G | 428 | −7.119 | 49.443 | −6.535 | 1.00 | 14.28 | | N |
| ATOM | 2774 | CA | ASN G | 428 | −8.080 | 49.830 | −5.505 | 1.00 | 14.60 | | C |
| ATOM | 2775 | CB | ASN G | 428 | −9.009 | 50.934 | −6.020 | 1.00 | 14.57 | | C |
| ATOM | 2776 | CG | ASN G | 428 | −8.353 | 51.808 | −7.071 | 1.00 | 13.24 | | C |
| ATOM | 2777 | OD1 | ASN G | 428 | −7.314 | 52.420 | −6.823 | 1.00 | 9.74 | | O |
| ATOM | 2778 | ND2 | ASN G | 428 | −9.009 | 51.946 | −8.217 | 1.00 | 12.83 | | N |
| ATOM | 2779 | C | ASN G | 428 | −8.914 | 48.658 | −4.994 | 1.00 | 14.83 | | C |
| ATOM | 2780 | O | ASN G | 428 | −9.645 | 48.793 | −4.013 | 1.00 | 15.25 | | O |
| ATOM | 2781 | N | ARG G | 429 | −8.920 | 47.564 | −5.750 | 1.00 | 14.78 | | N |
| ATOM | 2782 | CA | ARG G | 429 | −9.725 | 46.395 | −5.407 | 1.00 | 14.74 | | C |
| ATOM | 2783 | CB | ARG G | 429 | −10.088 | 46.405 | −3.920 | 1.00 | 14.93 | | C |
| ATOM | 2784 | CG | ARG G | 429 | −8.949 | 45.999 | −2.999 | 1.00 | 16.26 | | C |
| ATOM | 2785 | CD | ARG G | 429 | −8.954 | 44.502 | −2.732 | 1.00 | 19.79 | | C |
| ATOM | 2786 | NE | ARG G | 429 | −7.694 | 44.046 | −2.152 | 1.00 | 22.68 | | N |
| ATOM | 2787 | CZ | ARG G | 429 | −7.270 | 44.369 | −0.935 | 1.00 | 23.54 | | C |
| ATOM | 2788 | NH1 | ARG G | 429 | −7.998 | 45.170 | −0.168 | 1.00 | 22.87 | | N |
| ATOM | 2789 | NH2 | ARG G | 429 | −6.107 | 43.912 | −0.492 | 1.00 | 23.53 | | N |
| ATOM | 2790 | C | ARG G | 429 | −10.989 | 46.304 | −6.256 | 1.00 | 14.35 | | C |
| ATOM | 2791 | O | ARG G | 429 | −11.653 | 45.268 | −6.287 | 1.00 | 14.62 | | O |
| ATOM | 2792 | N | GLY G | 430 | −11.306 | 47.386 | −6.961 | 1.00 | 13.67 | | N |
| ATOM | 2793 | CA | GLY G | 430 | −12.201 | 47.311 | −8.110 | 1.00 | 12.80 | | C |
| ATOM | 2794 | C | GLY G | 430 | −11.870 | 46.136 | −9.009 | 1.00 | 12.11 | | C |
| ATOM | 2795 | O | GLY G | 430 | −10.707 | 45.903 | −9.338 | 1.00 | 12.18 | | O |
| ATOM | 2796 | N | ILE G | 431 | −12.892 | 45.374 | −9.380 | 1.00 | 11.58 | | N |
| ATOM | 2797 | CA | ILE G | 431 | −12.700 | 44.155 | −10.157 | 1.00 | 10.76 | | C |
| ATOM | 2798 | CB | ILE G | 431 | −13.777 | 43.106 | −9.812 | 1.00 | 10.43 | | C |
| ATOM | 2799 | CG1 | ILE G | 431 | −13.615 | 42.625 | −8.368 | 1.00 | 10.95 | | C |
| ATOM | 2800 | CD1 | ILE G | 431 | −14.920 | 42.259 | −7.695 | 1.00 | 12.08 | | C |
| ATOM | 2801 | CG2 | ILE G | 431 | −13.720 | 41.940 | −10.785 | 1.00 | 10.07 | | C |
| ATOM | 2802 | C | ILE G | 431 | −12.770 | 44.465 | −11.651 | 1.00 | 10.68 | | C |
| ATOM | 2803 | O | ILE G | 431 | −13.790 | 44.954 | −12.136 | 1.00 | 10.40 | | O |
| ATOM | 2804 | N | ILE G | 432 | −11.677 | 44.225 | −12.373 | 1.00 | 10.90 | | N |
| ATOM | 2805 | CA | ILE G | 432 | −11.658 | 44.482 | −13.815 | 1.00 | 11.42 | | C |
| ATOM | 2806 | CB | ILE G | 432 | −10.276 | 44.936 | −14.327 | 1.00 | 11.63 | | C |
| ATOM | 2807 | CG1 | ILE G | 432 | −9.411 | 45.460 | −13.180 | 1.00 | 12.68 | | C |
| ATOM | 2808 | CD1 | ILE G | 432 | −7.977 | 45.738 | −13.579 | 1.00 | 13.89 | | C |
| ATOM | 2809 | CG2 | ILE G | 432 | −10.432 | 45.992 | −15.412 | 1.00 | 12.24 | | C |
| ATOM | 2810 | C | ILE G | 432 | −12.118 | 43.290 | −14.648 | 1.00 | 11.41 | | C |
| ATOM | 2811 | O | ILE G | 432 | −12.834 | 43.463 | −15.635 | 1.00 | 11.63 | | O |
| ATOM | 2812 | N | LYS G | 433 | −11.505 | 42.136 | −14.405 | 1.00 | 11.52 | | N |
| ATOM | 2813 | CA | LYS G | 433 | −11.792 | 40.950 | −15.203 | 1.00 | 11.96 | | C |
| ATOM | 2814 | CB | LYS G | 433 | −10.516 | 40.392 | −15.837 | 1.00 | 12.31 | | C |
| ATOM | 2815 | CG | LYS G | 433 | −10.770 | 39.317 | −16.885 | 1.00 | 14.42 | | C |
| ATOM | 2816 | CD | LYS G | 433 | −9.723 | 39.348 | −17.987 | 1.00 | 16.74 | | C |
| ATOM | 2817 | CE | LYS G | 433 | −9.997 | 38.283 | −19.038 | 1.00 | 17.08 | | C |
| ATOM | 2818 | NZ | LYS G | 433 | −9.612 | 38.735 | −20.404 | 1.00 | 17.30 | | N |
| ATOM | 2819 | C | LYS G | 433 | −12.496 | 39.873 | −14.387 | 1.00 | 11.65 | | C |
| ATOM | 2820 | O | LYS G | 433 | −12.479 | 39.899 | −13.156 | 1.00 | 11.48 | | O |
| ATOM | 2821 | N | THR G | 434 | −13.143 | 38.946 | −15.086 | 1.00 | 11.28 | | N |
| ATOM | 2822 | CA | THR G | 434 | −13.816 | 37.821 | −14.445 | 1.00 | 11.21 | | C |
| ATOM | 2823 | CB | THR G | 434 | −15.222 | 38.216 | −13.955 | 1.00 | 11.30 | | C |
| ATOM | 2824 | OG1 | THR G | 434 | −15.963 | 37.037 | −13.619 | 1.00 | 11.83 | | O |
| ATOM | 2825 | CG2 | THR G | 434 | −15.966 | 38.988 | −15.035 | 1.00 | 11.84 | | C |
| ATOM | 2826 | C | THR G | 434 | −13.939 | 36.646 | −15.415 | 1.00 | 10.94 | | C |
| ATOM | 2827 | O | THR G | 434 | −14.257 | 36.836 | −16.589 | 1.00 | 10.93 | | O |
| ATOM | 2828 | N | PHE G | 435 | −13.628 | 35.442 | −14.939 | 1.00 | 11.10 | | N |
| ATOM | 2829 | CA | PHE G | 435 | −12.871 | 34.486 | −15.744 | 1.00 | 11.96 | | C |
| ATOM | 2830 | CB | PHE G | 435 | −11.673 | 33.929 | −14.977 | 1.00 | 11.51 | | C |
| ATOM | 2831 | CG | PHE G | 435 | −10.437 | 34.772 | −15.100 | 1.00 | 11.19 | | C |
| ATOM | 2832 | CD1 | PHE G | 435 | −9.910 | 35.409 | −13.989 | 1.00 | 10.86 | | C |
| ATOM | 2833 | CE1 | PHE G | 435 | −8.824 | 36.254 | −14.107 | 1.00 | 10.24 | | C |
| ATOM | 2834 | CZ | PHE G | 435 | −8.286 | 36.516 | −15.352 | 1.00 | 9.89 | | C |
| ATOM | 2835 | CE2 | PHE G | 435 | −8.834 | 35.927 | −16.474 | 1.00 | 10.78 | | C |
| ATOM | 2836 | CD2 | PHE G | 435 | −9.920 | 35.082 | −16.348 | 1.00 | 11.14 | | C |
| ATOM | 2837 | C | PHE G | 435 | −13.678 | 33.371 | −16.403 | 1.00 | 13.00 | | C |
| ATOM | 2838 | O | PHE G | 435 | −14.218 | 32.494 | −15.726 | 1.00 | 13.03 | | O |
| ATOM | 2839 | N | SER G | 436 | −13.506 | 33.272 | −17.717 | 1.00 | 13.89 | | N |
| ATOM | 2840 | CA | SER G | 436 | −14.276 | 32.356 | −18.546 | 1.00 | 14.67 | | C |
| ATOM | 2841 | CB | SER G | 436 | −14.391 | 32.910 | −19.969 | 1.00 | 15.04 | | C |
| ATOM | 2842 | OG | SER G | 436 | −15.702 | 33.377 | −20.235 | 1.00 | 16.58 | | O |
| ATOM | 2843 | C | SER G | 436 | −13.627 | 30.975 | −18.578 | 1.00 | 14.75 | | C |
| ATOM | 2844 | O | SER G | 436 | −13.081 | 30.559 | −19.601 | 1.00 | 14.75 | | O |
| ATOM | 2845 | N | ASN G | 437 | −13.713 | 30.256 | −17.465 | 1.00 | 14.44 | | N |
| ATOM | 2846 | CA | ASN G | 437 | −13.595 | 28.805 | −17.496 | 1.00 | 14.35 | | C |
| ATOM | 2847 | CB | ASN G | 437 | −14.935 | 28.179 | −17.896 | 1.00 | 14.75 | | C |
| ATOM | 2848 | CG | ASN G | 437 | −14.999 | 26.690 | −17.610 | 1.00 | 15.51 | | C |
| ATOM | 2849 | OD1 | ASN G | 437 | −15.013 | 26.275 | −16.450 | 1.00 | 16.85 | | O |
| ATOM | 2850 | ND2 | ASN G | 437 | −15.246 | 25.906 | −18.652 | 1.00 | 16.10 | | N |
| ATOM | 2851 | C | ASN G | 437 | −12.497 | 28.371 | −18.464 | 1.00 | 13.69 | | C |
| ATOM | 2852 | O | ASN G | 437 | −12.754 | 27.652 | −19.430 | 1.00 | 13.40 | | O |
| ATOM | 2853 | N | GLY G | 438 | −11.290 | 28.880 | −18.237 | 1.00 | 13.39 | | N |
| ATOM | 2854 | CA | GLY G | 438 | −10.183 | 28.701 | −19.170 | 1.00 | 12.92 | | C |
| ATOM | 2855 | C | GLY G | 438 | −8.839 | 28.941 | −18.510 | 1.00 | 12.72 | | C |
| ATOM | 2856 | O | GLY G | 438 | −8.620 | 28.538 | −17.367 | 1.00 | 12.83 | | O |
| ATOM | 2857 | N | CYS G | 439 | −7.971 | 29.677 | −19.195 | 1.00 | 12.17 | | N |
| ATOM | 2858 | CA | CYS G | 439 | −6.654 | 29.999 | −18.658 | 1.00 | 11.52 | | C |
| ATOM | 2859 | CB | CYS G | 439 | −5.696 | 28.822 | −18.844 | 1.00 | 11.68 | | C |
| ATOM | 2860 | SG | CYS G | 439 | −3.974 | 29.301 | −19.104 | 1.00 | 12.89 | | S |
| ATOM | 2861 | C | CYS G | 439 | −6.077 | 31.258 | −19.297 | 1.00 | 10.95 | | C |
| ATOM | 2862 | O | CYS G | 439 | −5.762 | 31.275 | −20.488 | 1.00 | 10.77 | | O |
| ATOM | 2863 | N | ASP G | 440 | −5.921 | 32.303 | −18.490 | 1.00 | 10.41 | | N |
| ATOM | 2864 | CA | ASP G | 440 | −5.546 | 33.619 | −18.993 | 1.00 | 10.06 | | C |
| ATOM | 2865 | CB | ASP G | 440 | −6.685 | 34.620 | −18.778 | 1.00 | 10.85 | | C |
| ATOM | 2866 | CG | ASP G | 440 | −7.418 | 34.956 | −20.062 | 1.00 | 13.11 | | C |
| ATOM | 2867 | OD1 | ASP G | 440 | −6.891 | 35.765 | −20.854 | 1.00 | 14.97 | | O |
| ATOM | 2868 | OD2 | ASP G | 440 | −8.559 | 34.479 | −20.237 | 1.00 | 15.27 | | O |
| ATOM | 2869 | C | ASP G | 440 | −4.276 | 34.131 | −18.318 | 1.00 | 9.01 | | C |
| ATOM | 2870 | O | ASP G | 440 | −3.793 | 33.542 | −17.350 | 1.00 | 9.09 | | O |
| ATOM | 2871 | N | TYR G | 441 | −3.806 | 35.287 | −18.778 | 1.00 | 7.92 | | N |
| ATOM | 2872 | CA | TYR G | 441 | −2.645 | 35.959 | −18.199 | 1.00 | 7.24 | | C |
| ATOM | 2873 | CB | TYR G | 441 | −1.457 | 35.871 | −19.165 | 1.00 | 7.04 | | C |
| ATOM | 2874 | CG | TYR G | 441 | −0.227 | 36.631 | −18.724 | 1.00 | 6.77 | | C |
| ATOM | 2875 | CD1 | TYR G | 441 | 0.762 | 36.008 | −17.974 | 1.00 | 7.41 | | C |
| ATOM | 2876 | CE1 | TYR G | 441 | 1.902 | 36.688 | −17.593 | 1.00 | 6.79 | | C |
| ATOM | 2877 | CZ | TYR G | 441 | 2.106 | 37.980 | −18.034 | 1.00 | 6.49 | | C |
| ATOM | 2878 | OH | TYR G | 441 | 3.230 | 38.670 | −17.635 | 1.00 | 5.09 | | O |
| ATOM | 2879 | CE2 | TYR G | 441 | 1.157 | 38.606 | −18.817 | 1.00 | 7.52 | | C |
| ATOM | 2880 | CD2 | TYR G | 441 | 0.017 | 37.918 | −19.186 | 1.00 | 7.12 | | C |
| ATOM | 2881 | C | TYR G | 441 | −3.005 | 37.421 | −17.946 | 1.00 | 6.89 | | C |
| ATOM | 2882 | O | TYR G | 441 | −3.809 | 38.000 | −18.677 | 1.00 | 7.12 | | O |
| ATOM | 2883 | N | VAL G | 442 | −2.452 | 37.999 | −16.886 | 1.00 | 6.73 | | N |
| ATOM | 2884 | CA | VAL G | 442 | −2.612 | 39.427 | −16.615 | 1.00 | 6.78 | | C |
| ATOM | 2885 | CB | VAL G | 442 | −3.614 | 39.671 | −15.472 | 1.00 | 6.51 | | C |
| ATOM | 2886 | CG1 | VAL G | 442 | −5.041 | 39.483 | −15.967 | 1.00 | 7.38 | | C |
| ATOM | 2887 | CG2 | VAL G | 442 | −3.322 | 38.742 | −14.303 | 1.00 | 6.00 | | C |
| ATOM | 2888 | C | VAL G | 442 | −1.262 | 40.025 | −16.234 | 1.00 | 7.13 | | C |
| ATOM | 2889 | O | VAL G | 442 | −0.272 | 39.301 | −16.134 | 1.00 | 7.77 | | O |
| ATOM | 2890 | N | SER G | 443 | −1.210 | 41.335 | −16.022 | 1.00 | 7.12 | | N |
| ATOM | 2891 | CA | SER G | 443 | 0.062 | 41.970 | −15.700 | 1.00 | 7.64 | | C |
| ATOM | 2892 | CB | SER G | 443 | 0.770 | 42.447 | −16.969 | 1.00 | 7.83 | | C |
| ATOM | 2893 | OG | SER G | 443 | 0.637 | 43.847 | −17.131 | 1.00 | 7.99 | | O |
| ATOM | 2894 | C | SER G | 443 | −0.051 | 43.104 | −14.690 | 1.00 | 8.16 | | C |
| ATOM | 2895 | O | SER G | 443 | −1.142 | 43.607 | −14.420 | 1.00 | 8.69 | | O |
| ATOM | 2896 | N | ASN G | 444 | 1.086 | 43.484 | −14.117 | 1.00 | 8.73 | | N |
| ATOM | 2897 | CA | ASN G | 444 | 1.139 | 44.600 | −13.184 | 1.00 | 9.50 | | C |
| ATOM | 2898 | CB | ASN G | 444 | 2.521 | 44.684 | −12.538 | 1.00 | 9.65 | | C |
| ATOM | 2899 | CG | ASN G | 444 | 3.256 | 43.357 | −12.565 | 1.00 | 10.64 | | C |
| ATOM | 2900 | OD1 | ASN G | 444 | 2.641 | 42.291 | −12.498 | 1.00 | 12.28 | | O |
| ATOM | 2901 | ND2 | ASN G | 444 | 4.578 | 43.416 | −12.685 | 1.00 | 11.75 | | N |
| ATOM | 2902 | C | ASN G | 444 | 0.791 | 45.922 | −13.857 | 1.00 | 10.24 | | C |
| ATOM | 2903 | O | ASN G | 444 | 1.086 | 46.126 | −15.034 | 1.00 | 9.89 | | O |
| ATOM | 2904 | N | LYS G | 445 | 0.266 | 46.855 | −13.069 | 1.00 | 11.63 | | N |
| ATOM | 2905 | CA | LYS G | 445 | −0.752 | 47.787 | −13.545 | 1.00 | 13.07 | | C |
| ATOM | 2906 | CB | LYS G | 445 | −0.107 | 49.048 | −14.123 | 1.00 | 13.70 | | C |
| ATOM | 2907 | CG | LYS G | 445 | −1.068 | 50.216 | −14.307 | 1.00 | 15.80 | | C |
| ATOM | 2908 | CD | LYS G | 445 | −2.384 | 49.986 | −13.575 | 1.00 | 18.31 | | C |
| ATOM | 2909 | CE | LYS G | 445 | −2.166 | 49.766 | −12.086 | 1.00 | 19.03 | | C |
| ATOM | 2910 | NZ | LYS G | 445 | −1.048 | 50.592 | −11.554 | 1.00 | 20.02 | | N |
| ATOM | 2911 | C | LYS G | 445 | −1.681 | 47.147 | −14.572 | 1.00 | 12.96 | | C |
| ATOM | 2912 | O | LYS G | 445 | −1.258 | 46.313 | −15.372 | 1.00 | 13.24 | | O |
| ATOM | 2913 | N | GLY G | 446 | −2.943 | 47.559 | −14.558 | 1.00 | 13.08 | | N |
| ATOM | 2914 | CA | GLY G | 446 | −4.051 | 46.616 | −14.597 | 1.00 | 12.42 | | C |
| ATOM | 2915 | C | GLY G | 446 | −4.212 | 45.900 | −13.272 | 1.00 | 11.83 | | C |
| ATOM | 2916 | O | GLY G | 446 | −4.832 | 46.426 | −12.347 | 1.00 | 12.44 | | O |
| ATOM | 2917 | N | VAL G | 447 | −3.517 | 44.777 | −13.126 | 1.00 | 10.63 | | N |
| ATOM | 2918 | CA | VAL G | 447 | −3.875 | 43.795 | −12.114 | 1.00 | 10.01 | | C |
| ATOM | 2919 | CB | VAL G | 447 | −3.898 | 42.365 | −12.681 | 1.00 | 9.97 | | C |
| ATOM | 2920 | CG1 | VAL G | 447 | −4.591 | 41.422 | −11.708 | 1.00 | 10.51 | | C |
| ATOM | 2921 | CG2 | VAL G | 447 | −4.594 | 42.345 | −14.033 | 1.00 | 10.50 | | C |
| ATOM | 2922 | C | VAL G | 447 | −2.978 | 43.851 | −10.882 | 1.00 | 9.53 | | C |
| ATOM | 2923 | O | VAL G | 447 | −1.761 | 44.008 | −10.985 | 1.00 | 9.61 | | O |
| ATOM | 2924 | N | ASP G | 448 | −3.586 | 43.575 | −9.735 | 1.00 | 9.16 | | N |
| ATOM | 2925 | CA | ASP G | 448 | −2.956 | 43.763 | −8.437 | 1.00 | 8.75 | | C |
| ATOM | 2926 | CB | ASP G | 448 | −3.515 | 45.022 | −7.771 | 1.00 | 9.22 | | C |
| ATOM | 2927 | CG | ASP G | 448 | −2.579 | 45.599 | −6.729 | 1.00 | 11.44 | | C |
| ATOM | 2928 | OD1 | ASP G | 448 | −1.725 | 44.849 | −6.212 | 1.00 | 13.83 | | O |
| ATOM | 2929 | OD2 | ASP G | 448 | −2.713 | 46.799 | −6.409 | 1.00 | 14.13 | | O |
| ATOM | 2930 | C | ASP G | 448 | −3.313 | 42.546 | −7.598 | 1.00 | 8.18 | | C |
| ATOM | 2931 | O | ASP G | 448 | −2.555 | 42.122 | −6.725 | 1.00 | 8.06 | | O |
| ATOM | 2932 | N | THR G | 449 | −4.451 | 41.949 | −7.933 | 1.00 | 7.63 | | N |
| ATOM | 2933 | CA | THR G | 449 | −4.991 | 40.823 | −7.192 | 1.00 | 6.80 | | C |
| ATOM | 2934 | CB | THR G | 449 | −5.988 | 41.293 | −6.116 | 1.00 | 6.89 | | C |
| ATOM | 2935 | OG1 | THR G | 449 | −5.284 | 41.584 | −4.902 | 1.00 | 6.16 | | O |
| ATOM | 2936 | CG2 | THR G | 449 | −7.032 | 40.219 | −5.848 | 1.00 | 7.55 | | C |
| ATOM | 2937 | C | THR G | 449 | −5.717 | 39.906 | −8.164 | 1.00 | 6.53 | | C |
| ATOM | 2938 | O | THR G | 449 | −6.385 | 40.370 | −9.087 | 1.00 | 6.59 | | O |
| ATOM | 2939 | N | VAL G | 450 | −5.536 | 38.603 | −7.989 | 1.00 | 6.07 | | N |
| ATOM | 2940 | CA | VAL G | 450 | −6.531 | 37.646 | −8.434 | 1.00 | 6.02 | | C |
| ATOM | 2941 | CB | VAL G | 450 | −5.981 | 36.723 | −9.535 | 1.00 | 5.61 | | C |
| ATOM | 2942 | CG1 | VAL G | 450 | −5.377 | 37.545 | −10.663 | 1.00 | 5.32 | | C |
| ATOM | 2943 | CG2 | VAL G | 450 | −4.954 | 35.761 | −8.958 | 1.00 | 5.55 | | C |
| ATOM | 2944 | C | VAL G | 450 | −7.021 | 36.811 | −7.263 | 1.00 | 6.37 | | C |
| ATOM | 2945 | O | VAL G | 450 | −6.299 | 36.609 | −6.287 | 1.00 | 6.54 | | O |
| ATOM | 2946 | N | SER G | 451 | −8.307 | 36.486 | −7.288 | 1.00 | 6.41 | | N |
| ATOM | 2947 | CA | SER G | 451 | −8.857 | 35.507 | −6.370 | 1.00 | 6.70 | | C |
| ATOM | 2948 | CB | SER G | 451 | −9.995 | 36.120 | −5.553 | 1.00 | 7.13 | | C |
| ATOM | 2949 | OG | SER G | 451 | −10.537 | 37.254 | −6.207 | 1.00 | 9.26 | | O |
| ATOM | 2950 | C | SER G | 451 | −9.354 | 34.292 | −7.139 | 1.00 | 6.47 | | C |
| ATOM | 2951 | O | SER G | 451 | −9.896 | 34.423 | −8.238 | 1.00 | 6.54 | | O |
| ATOM | 2952 | N | VAL G | 452 | −8.962 | 33.116 | −6.664 | 1.00 | 6.56 | | N |
| ATOM | 2953 | CA | VAL G | 452 | −9.578 | 31.868 | −7.091 | 1.00 | 6.31 | | C |
| ATOM | 2954 | CB | VAL G | 452 | −8.516 | 30.849 | −7.542 | 1.00 | 5.74 | | C |
| ATOM | 2955 | CG1 | VAL G | 452 | −9.170 | 29.688 | −8.275 | 1.00 | 5.06 | | C |
| ATOM | 2956 | CG2 | VAL G | 452 | −7.471 | 31.523 | −8.419 | 1.00 | 5.56 | | C |
| ATOM | 2957 | C | VAL G | 452 | −10.381 | 31.264 | −5.944 | 1.00 | 6.60 | | C |
| ATOM | 2958 | O | VAL G | 452 | −9.861 | 31.079 | −4.844 | 1.00 | 7.04 | | O |
| ATOM | 2959 | N | GLY G | 453 | −11.652 | 30.975 | −6.198 | 1.00 | 6.54 | | N |
| ATOM | 2960 | CA | GLY G | 453 | −12.581 | 30.650 | −5.124 | 1.00 | 6.43 | | C |
| ATOM | 2961 | C | GLY G | 453 | −12.375 | 31.548 | −3.920 | 1.00 | 6.09 | | C |
| ATOM | 2962 | O | GLY G | 453 | −12.581 | 32.759 | −3.999 | 1.00 | 5.90 | | O |
| ATOM | 2963 | N | ASN G | 454 | −11.872 | 30.971 | −2.833 | 1.00 | 6.02 | | N |
| ATOM | 2964 | CA | ASN G | 454 | −11.672 | 31.727 | −1.603 | 1.00 | 6.39 | | C |
| ATOM | 2965 | CB | ASN G | 454 | −12.335 | 31.027 | −0.420 | 1.00 | 6.81 | | C |
| ATOM | 2966 | CG | ASN G | 454 | −13.717 | 31.567 | −0.135 | 1.00 | 8.99 | | C |
| ATOM | 2967 | OD1 | ASN G | 454 | −13.867 | 32.523 | 0.625 | 1.00 | 11.67 | | O |
| ATOM | 2968 | ND2 | ASN G | 454 | −14.693 | 31.112 | −0.911 | 1.00 | 10.25 | | N |
| ATOM | 2969 | C | ASN G | 454 | −10.211 | 32.020 | −1.296 | 1.00 | 6.15 | | C |
| ATOM | 2970 | O | ASN G | 454 | −9.900 | 32.765 | −0.367 | 1.00 | 6.47 | | O |
| ATOM | 2971 | N | THR G | 455 | −9.322 | 31.491 | −2.128 | 1.00 | 5.40 | | N |
| ATOM | 2972 | CA | THR G | 455 | −7.912 | 31.841 | −2.053 | 1.00 | 4.53 | | C |
| ATOM | 2973 | CB | THR G | 455 | −7.033 | 30.774 | −2.724 | 1.00 | 4.70 | | C |
| ATOM | 2974 | OG1 | THR G | 455 | −7.344 | 29.486 | −2.180 | 1.00 | 5.28 | | O |
| ATOM | 2975 | CG2 | THR G | 455 | −5.560 | 31.074 | −2.491 | 1.00 | 5.57 | | C |
| ATOM | 2976 | C | THR G | 455 | −7.659 | 33.187 | −2.717 | 1.00 | 4.01 | | C |
| ATOM | 2977 | O | THR G | 455 | −8.312 | 33.540 | −3.698 | 1.00 | 3.78 | | O |
| ATOM | 2978 | N | LEU G | 456 | −6.716 | 33.943 | −2.165 | 1.00 | 3.87 | | N |
| ATOM | 2979 | CA | LEU G | 456 | −6.355 | 35.237 | −2.726 | 1.00 | 4.05 | | C |
| ATOM | 2980 | CB | LEU G | 456 | −6.684 | 36.360 | −1.742 | 1.00 | 3.95 | | C |
| ATOM | 2981 | CG | LEU G | 456 | −6.595 | 37.779 | −2.306 | 1.00 | 3.76 | | C |
| ATOM | 2982 | CD1 | LEU G | 456 | −7.572 | 37.962 | −3.457 | 1.00 | 5.35 | | C |
| ATOM | 2983 | CD2 | LEU G | 456 | −6.845 | 38.809 | −1.216 | 1.00 | 4.10 | | C |
| ATOM | 2984 | C | LEU G | 456 | −4.879 | 35.288 | −3.105 | 1.00 | 4.47 | | C |
| ATOM | 2985 | O | LEU G | 456 | −4.006 | 35.028 | −2.276 | 1.00 | 4.94 | | O |
| ATOM | 2986 | N | TYR G | 457 | −4.609 | 35.652 | −4.355 | 1.00 | 4.59 | | N |
| ATOM | 2987 | CA | TYR G | 457 | −3.242 | 35.786 | −4.849 | 1.00 | 4.60 | | C |
| ATOM | 2988 | CB | TYR G | 457 | −3.056 | 34.938 | −6.113 | 1.00 | 4.71 | | C |
| ATOM | 2989 | CG | TYR G | 457 | −3.201 | 33.447 | −5.888 | 1.00 | 5.62 | | C |
| ATOM | 2990 | CD1 | TYR G | 457 | −4.451 | 32.838 | −5.896 | 1.00 | 7.09 | | C |
| ATOM | 2991 | CE1 | TYR G | 457 | −4.582 | 31.473 | −5.712 | 1.00 | 8.22 | | C |
| ATOM | 2992 | CZ | TYR G | 457 | −3.456 | 30.699 | −5.533 | 1.00 | 8.06 | | C |
| ATOM | 2993 | OH | TYR G | 457 | −3.580 | 29.341 | −5.349 | 1.00 | 8.80 | | O |
| ATOM | 2994 | CE2 | TYR G | 457 | −2.204 | 31.276 | −5.547 | 1.00 | 7.18 | | C |
| ATOM | 2995 | CD2 | TYR G | 457 | −2.083 | 32.639 | −5.735 | 1.00 | 6.66 | | C |
| ATOM | 2996 | C | TYR G | 457 | −2.924 | 37.256 | −5.152 | 1.00 | 4.46 | | C |
| ATOM | 2997 | O | TYR G | 457 | −3.742 | 37.959 | −5.745 | 1.00 | 4.92 | | O |
| ATOM | 2998 | N | TYR G | 458 | −1.727 | 37.706 | −4.772 | 1.00 | 3.90 | | N |
| ATOM | 2999 | CA | TYR G | 458 | −1.312 | 39.118 | −4.919 | 1.00 | 3.26 | | C |
| ATOM | 3000 | CB | TYR G | 458 | −0.740 | 39.635 | −3.582 | 1.00 | 3.16 | | C |
| ATOM | 3001 | CG | TYR G | 458 | −1.716 | 39.659 | −2.411 | 1.00 | 3.29 | | C |
| ATOM | 3002 | CD1 | TYR G | 458 | −1.782 | 38.602 | −1.509 | 1.00 | 3.93 | | C |
| ATOM | 3003 | CE1 | TYR G | 458 | −2.632 | 38.646 | −0.412 | 1.00 | 4.90 | | C |
| ATOM | 3004 | CZ | TYR G | 458 | −3.395 | 39.773 | −0.185 | 1.00 | 5.12 | | C |
| ATOM | 3005 | OH | TYR G | 458 | −4.250 | 39.817 | 0.893 | 1.00 | 5.42 | | O |
| ATOM | 3006 | CE2 | TYR G | 458 | −3.311 | 40.852 | −1.039 | 1.00 | 4.72 | | C |
| ATOM | 3007 | CD2 | TYR G | 458 | −2.457 | 40.801 | −2.128 | 1.00 | 3.89 | | C |
| ATOM | 3008 | C | TYR G | 458 | −0.248 | 39.290 | −6.043 | 1.00 | 3.04 | | C |
| ATOM | 3009 | O | TYR G | 458 | 0.749 | 38.569 | −6.041 | 1.00 | 3.37 | | O |
| ATOM | 3010 | N | VAL G | 459 | −0.351 | 40.348 | −6.866 | 1.00 | 2.71 | | N |
| ATOM | 3011 | CA | VAL G | 459 | 0.376 | 40.435 | −8.177 | 1.00 | 2.61 | | C |
| ATOM | 3012 | CB | VAL G | 459 | −0.589 | 40.554 | −9.380 | 1.00 | 2.22 | | C |
| ATOM | 3013 | CG1 | VAL G | 459 | 0.154 | 40.287 | −10.682 | 1.00 | 2.62 | | C |
| ATOM | 3014 | CG2 | VAL G | 459 | −1.756 | 39.589 | −9.224 | 1.00 | 2.61 | | C |
| ATOM | 3015 | C | VAL G | 459 | 1.609 | 41.392 | −8.360 | 1.00 | 3.03 | | C |
| ATOM | 3016 | O | VAL G | 459 | 2.575 | 41.281 | −7.604 | 1.00 | 3.21 | | O |
| ATOM | 3017 | N | ASN G | 460 | 1.708 | 42.037 | −9.537 | 1.00 | 106.93 | | N |
| ATOM | 3018 | CA | ASN G | 460 | 2.714 | 43.111 | −9.870 | 1.00 | 95.54 | | C |
| ATOM | 3019 | CB | ASN G | 460 | 3.598 | 43.589 | −8.700 | 1.00 | 20.00 | | C |
| ATOM | 3020 | CG | ASN G | 460 | 4.310 | 44.931 | −9.000 | 1.00 | 20.00 | | C |
| ATOM | 3021 | OD1 | ASN G | 460 | 3.713 | 45.851 | −9.563 | 1.00 | 20.00 | | O |
| ATOM | 3022 | ND2 | ASN G | 460 | 5.548 | 45.065 | −8.532 | 1.00 | 20.00 | | N |
| ATOM | 3023 | C | ASN G | 460 | 3.403 | 43.322 | −11.247 | 1.00 | 99.41 | | C |
| ATOM | 3024 | O | ASN G | 460 | 3.869 | 44.430 | −11.523 | 1.00 | 108.91 | | O |
| ATOM | 3025 | N | LYS G | 461 | 3.568 | 42.271 | −12.055 | 1.00 | 97.04 | | N |
| ATOM | 3026 | CA | LYS G | 461 | 4.464 | 42.327 | −13.236 | 1.00 | 105.93 | | C |
| ATOM | 3027 | CB | LYS G | 461 | 5.335 | 41.068 | −13.312 | 1.00 | 20.00 | | C |
| ATOM | 3028 | CG | LYS G | 461 | 6.382 | 40.951 | −12.209 | 1.00 | 20.00 | | C |
| ATOM | 3029 | CD | LYS G | 461 | 7.131 | 39.623 | −12.296 | 1.00 | 20.00 | | C |
| ATOM | 3030 | CE | LYS G | 461 | 8.255 | 39.536 | −11.268 | 1.00 | 20.00 | | C |
| ATOM | 3031 | NZ | LYS G | 461 | 8.886 | 38.184 | −11.223 | 1.00 | 20.00 | | N |
| ATOM | 3032 | C | LYS G | 461 | 3.727 | 42.546 | −14.573 | 1.00 | 102.28 | | C |
| ATOM | 3033 | O | LYS G | 461 | 2.507 | 42.702 | −14.590 | 1.00 | 105.02 | | O |
| ATOM | 3034 | N | GLN G | 462 | 4.464 | 42.556 | −15.685 | 1.00 | 96.68 | | N |
| ATOM | 3035 | CA | GLN G | 462 | 3.854 | 42.633 | −17.020 | 1.00 | 105.71 | | C |
| ATOM | 3036 | CB | GLN G | 462 | 3.988 | 44.047 | −17.593 | 1.00 | 20.00 | | C |
| ATOM | 3037 | CG | GLN G | 462 | 3.237 | 45.115 | −16.814 | 1.00 | 20.00 | | C |
| ATOM | 3038 | CD | GLN G | 462 | 3.484 | 46.512 | −17.353 | 1.00 | 20.00 | | C |
| ATOM | 3039 | OE1 | GLN G | 462 | 4.527 | 46.786 | −17.947 | 1.00 | 20.00 | | O |
| ATOM | 3040 | NE2 | GLN G | 462 | 2.529 | 47.408 | −17.135 | 1.00 | 20.00 | | N |
| ATOM | 3041 | C | GLN G | 462 | 4.479 | 41.626 | −17.986 | 1.00 | 101.50 | | C |
| ATOM | 3042 | O | GLN G | 462 | 5.701 | 41.501 | −18.051 | 1.00 | 105.94 | | O |
| ATOM | 3043 | N | GLU G | 463 | 3.646 | 40.946 | −18.769 | 1.00 | 98.92 | | N |
| ATOM | 3044 | CA | GLU G | 463 | 4.140 | 39.928 | −19.693 | 1.00 | 108.59 | | C |
| ATOM | 3045 | CB | GLU G | 463 | 3.380 | 38.612 | −19.512 | 1.00 | 20.00 | | C |
| ATOM | 3046 | CG | GLU G | 463 | 3.682 | 37.900 | −18.201 | 1.00 | 20.00 | | C |
| ATOM | 3047 | CD | GLU G | 463 | 2.842 | 36.650 | −18.005 | 1.00 | 20.00 | | C |
| ATOM | 3048 | OE1 | GLU G | 463 | 2.003 | 36.350 | −18.881 | 1.00 | 20.00 | | O |
| ATOM | 3049 | OE2 | GLU G | 463 | 3.075 | 35.929 | −17.011 | 1.00 | 20.00 | | O |
| ATOM | 3050 | C | GLU G | 463 | 4.074 | 40.384 | −21.146 | 1.00 | 116.39 | | C |
| ATOM | 3051 | O | GLU G | 463 | 3.025 | 40.814 | −21.627 | 1.00 | 118.70 | | O |
| ATOM | 3052 | N | GLY G | 464 | 5.200 | 40.272 | −21.845 | 1.00 | 127.70 | | N |
| ATOM | 3053 | CA | GLY G | 464 | 5.261 | 40.610 | −23.261 | 1.00 | 143.17 | | C |
| ATOM | 3054 | C | GLY G | 464 | 4.710 | 39.509 | −24.146 | 1.00 | 149.77 | | C |
| ATOM | 3055 | O | GLY G | 464 | 4.443 | 38.400 | −23.681 | 1.00 | 155.06 | | O |
| ATOM | 3056 | N | LYS G | 465 | 4.544 | 39.816 | −25.428 | 1.00 | 153.73 | | N |
| ATOM | 3057 | CA | LYS G | 465 | 3.994 | 38.862 | −26.384 | 1.00 | 153.69 | | C |
| ATOM | 3058 | CB | LYS G | 465 | 4.816 | 37.571 | −26.389 | 1.00 | 20.00 | | C |
| ATOM | 3059 | CG | LYS G | 465 | 6.228 | 37.735 | −26.927 | 1.00 | 20.00 | | C |
| ATOM | 3060 | CD | LYS G | 465 | 6.996 | 36.424 | −26.873 | 1.00 | 20.00 | | C |
| ATOM | 3061 | CE | LYS G | 465 | 8.402 | 36.585 | −27.426 | 1.00 | 20.00 | | C |
| ATOM | 3062 | NZ | LYS G | 465 | 9.172 | 35.312 | −27.363 | 1.00 | 20.00 | | N |
| ATOM | 3063 | C | LYS G | 465 | 2.532 | 38.552 | −26.080 | 1.00 | 155.37 | | C |
| ATOM | 3064 | O | LYS G | 465 | 1.710 | 38.437 | −26.988 | 1.00 | 155.20 | | O |
| ATOM | 3065 | N | GLU G | 472 | −0.074 | 35.733 | −32.259 | 1.00 | 145.47 | | N |
| ATOM | 3066 | CA | GLU G | 472 | −0.706 | 34.866 | −33.247 | 1.00 | 151.12 | | C |
| ATOM | 3067 | CB | GLU G | 472 | 0.178 | 34.736 | −34.490 | 1.00 | 20.00 | | C |
| ATOM | 3068 | CG | GLU G | 472 | 0.339 | 36.028 | −35.276 | 1.00 | 20.00 | | C |
| ATOM | 3069 | CD | GLU G | 472 | 1.271 | 35.876 | −36.463 | 1.00 | 20.00 | | C |
| ATOM | 3070 | OE1 | GLU G | 472 | 1.954 | 34.835 | −36.556 | 1.00 | 20.00 | | O |
| ATOM | 3071 | OE2 | GLU G | 472 | 1.325 | 36.803 | −37.299 | 1.00 | 20.00 | | O |
| ATOM | 3072 | C | GLU G | 472 | −0.993 | 33.486 | −32.663 | 1.00 | 153.35 | | C |
| ATOM | 3073 | O | GLU G | 472 | −0.069 | 32.755 | −32.306 | 1.00 | 159.41 | | O |
| ATOM | 3074 | N | PRO G | 473 | −2.282 | 33.131 | −32.557 | 1.00 | 150.51 | | N |
| ATOM | 3075 | CA | PRO G | 473 | −2.668 | 31.819 | −32.081 | 1.00 | 141.97 | | C |
| ATOM | 3076 | CB | PRO G | 473 | −3.684 | 32.152 | −30.990 | 1.00 | 20.00 | | C |
| ATOM | 3077 | CG | PRO G | 473 | −4.311 | 33.467 | −31.446 | 1.00 | 20.00 | | C |
| ATOM | 3078 | CD | PRO G | 473 | −3.423 | 34.059 | −32.523 | 1.00 | 20.00 | | C |
| ATOM | 3079 | C | PRO G | 473 | −3.357 | 31.051 | −33.200 | 1.00 | 135.14 | | C |
| ATOM | 3080 | O | PRO G | 473 | −3.883 | 31.662 | −34.131 | 1.00 | 144.93 | | O |
| ATOM | 3081 | N | ILE G | 474 | −3.379 | 29.727 | −33.098 | 1.00 | 118.07 | | N |
| ATOM | 3082 | CA | ILE G | 474 | −3.969 | 28.899 | −34.142 | 1.00 | 105.14 | | C |
| ATOM | 3083 | CB | ILE G | 474 | −3.193 | 29.027 | −35.469 | 1.00 | 20.00 | | C |
| ATOM | 3084 | CG1 | ILE G | 474 | −3.315 | 30.446 | −36.028 | 1.00 | 20.00 | | C |
| ATOM | 3085 | CD1 | ILE G | 474 | −2.352 | 30.745 | −37.156 | 1.00 | 20.00 | | C |
| ATOM | 3086 | CG2 | ILE G | 474 | −3.696 | 28.008 | −36.481 | 1.00 | 20.00 | | C |
| ATOM | 3087 | C | ILE G | 474 | −4.007 | 27.432 | −33.730 | 1.00 | 104.75 | | C |
| ATOM | 3088 | O | ILE G | 474 | −3.033 | 26.702 | −33.915 | 1.00 | 111.83 | | O |
| ATOM | 3089 | N | ILE G | 475 | −5.149 | 26.992 | −33.213 | 1.00 | 94.65 | | N |
| ATOM | 3090 | CA | ILE G | 475 | −5.249 | 25.667 | −32.611 | 1.00 | 79.88 | | C |
| ATOM | 3091 | CB | ILE G | 475 | −5.443 | 25.753 | −31.087 | 1.00 | 20.00 | | C |
| ATOM | 3092 | CG1 | ILE G | 475 | −4.165 | 26.260 | −30.413 | 1.00 | 20.00 | | C |
| ATOM | 3093 | CD1 | ILE G | 475 | −4.414 | 27.012 | −29.122 | 1.00 | 20.00 | | C |
| ATOM | 3094 | CG2 | ILE G | 475 | −5.873 | 24.406 | −30.525 | 1.00 | 20.00 | | C |
| ATOM | 3095 | C | ILE G | 475 | −6.374 | 24.834 | −33.223 | 1.00 | 79.52 | | C |
| ATOM | 3096 | O | ILE G | 475 | −7.554 | 25.117 | −33.016 | 1.00 | 82.70 | | O |
| ATOM | 3097 | N | ASN G | 476 | −5.996 | 23.761 | −33.912 | 1.00 | 69.64 | | N |
| ATOM | 3098 | CA | ASN G | 476 | −6.962 | 22.821 | −34.472 | 1.00 | 68.88 | | C |
| ATOM | 3099 | CB | ASN G | 476 | −6.365 | 22.100 | −35.683 | 1.00 | 20.00 | | C |
| ATOM | 3100 | CG | ASN G | 476 | −6.170 | 23.019 | −36.872 | 1.00 | 20.00 | | C |
| ATOM | 3101 | OD1 | ASN G | 476 | −6.855 | 24.032 | −37.009 | 1.00 | 20.00 | | O |
| ATOM | 3102 | ND2 | ASN G | 476 | −5.251 | 22.651 | −37.756 | 1.00 | 20.00 | | N |
| ATOM | 3103 | C | ASN G | 476 | −7.434 | 21.799 | −33.443 | 1.00 | 67.13 | | C |
| ATOM | 3104 | O | ASN G | 476 | −6.628 | 21.229 | −32.707 | 1.00 | 73.10 | | O |
| ATOM | 3105 | N | PHE G | 477 | −8.735 | 21.527 | −33.438 | 1.00 | 64.95 | | N |
| ATOM | 3106 | CA | PHE G | 477 | −9.327 | 20.624 | −32.455 | 1.00 | 68.13 | | C |
| ATOM | 3107 | CB | PHE G | 477 | −10.854 | 20.711 | −32.493 | 1.00 | 20.00 | | C |
| ATOM | 3108 | CG | PHE G | 477 | −11.397 | 22.047 | −32.079 | 1.00 | 20.00 | | C |
| ATOM | 3109 | CD1 | PHE G | 477 | −11.580 | 22.352 | −30.740 | 1.00 | 20.00 | | C |
| ATOM | 3110 | CE1 | PHE G | 477 | −12.086 | 23.580 | −30.357 | 1.00 | 20.00 | | C |
| ATOM | 3111 | CZ | PHE G | 477 | −12.426 | 24.514 | −31.316 | 1.00 | 20.00 | | C |
| ATOM | 3112 | CE2 | PHE G | 477 | −12.257 | 24.219 | −32.654 | 1.00 | 20.00 | | C |
| ATOM | 3113 | CD2 | PHE G | 477 | −11.750 | 22.989 | −33.030 | 1.00 | 20.00 | | C |
| ATOM | 3114 | C | PHE G | 477 | −8.890 | 19.180 | −32.672 | 1.00 | 65.57 | | C |
| ATOM | 3115 | O | PHE G | 477 | −9.270 | 18.290 | −31.911 | 1.00 | 49.83 | | O |
| ATOM | 3116 | N | TYR G | 478 | −8.204 | 18.931 | −33.782 | 1.00 | 51.97 | | N |
| ATOM | 3117 | CA | TYR G | 478 | −7.618 | 17.619 | −34.036 | 1.00 | 52.01 | | C |
| ATOM | 3118 | CB | TYR G | 478 | −7.356 | 17.428 | −35.530 | 1.00 | 20.00 | | C |
| ATOM | 3119 | CG | TYR G | 478 | −8.609 | 17.426 | −36.372 | 1.00 | 20.00 | | C |
| ATOM | 3120 | CD1 | TYR G | 478 | −9.337 | 16.259 | −36.567 | 1.00 | 20.00 | | C |
| ATOM | 3121 | CE1 | TYR G | 478 | −10.489 | 16.255 | −37.329 | 1.00 | 20.00 | | C |
| ATOM | 3122 | CZ | TYR G | 478 | −10.930 | 17.429 | −37.902 | 1.00 | 20.00 | | C |
| ATOM | 3123 | OH | TYR G | 478 | −12.078 | 17.432 | −38.658 | 1.00 | 20.00 | | O |
| ATOM | 3124 | CE2 | TYR G | 478 | −10.230 | 18.602 | −37.717 | 1.00 | 20.00 | | C |
| ATOM | 3125 | CD2 | TYR G | 478 | −9.079 | 18.596 | −36.954 | 1.00 | 20.00 | | C |
| ATOM | 3126 | C | TYR G | 478 | −6.325 | 17.459 | −33.251 | 1.00 | 66.63 | | C |
| ATOM | 3127 | O | TYR G | 478 | −5.548 | 16.535 | −33.492 | 1.00 | 63.45 | | O |
| ATOM | 3128 | N | ASP G | 479 | −6.062 | 18.423 | −32.375 | 1.00 | 65.34 | | N |
| ATOM | 3129 | CA | ASP G | 479 | −4.928 | 18.351 | −31.469 | 1.00 | 65.32 | | C |
| ATOM | 3130 | CB | ASP G | 479 | −4.205 | 19.698 | −31.413 | 1.00 | 20.00 | | C |
| ATOM | 3131 | CG | ASP G | 479 | −3.509 | 20.038 | −32.717 | 1.00 | 20.00 | | C |
| ATOM | 3132 | OD1 | ASP G | 479 | −3.293 | 19.119 | −33.534 | 1.00 | 20.00 | | O |
| ATOM | 3133 | OD2 | ASP G | 479 | −3.184 | 21.225 | −32.930 | 1.00 | 20.00 | | O |
| ATOM | 3134 | C | ASP G | 479 | −5.383 | 17.937 | −30.076 | 1.00 | 64.83 | | C |
| ATOM | 3135 | O | ASP G | 479 | −4.561 | 17.574 | −29.236 | 1.00 | 60.42 | | O |
| ATOM | 3136 | N | PRO G | 480 | −6.707 | 17.893 | −29.861 | 1.00 | 47.60 | | N |
| ATOM | 3137 | CA | PRO G | 480 | −7.262 | 17.523 | −28.572 | 1.00 | 46.52 | | C |
| ATOM | 3138 | CB | PRO G | 480 | −8.509 | 18.400 | −28.480 | 1.00 | 20.00 | | C |
| ATOM | 3139 | CG | PRO G | 480 | −8.954 | 18.565 | −29.904 | 1.00 | 20.00 | | C |
| ATOM | 3140 | CD | PRO G | 480 | −7.755 | 18.332 | −30.799 | 1.00 | 20.00 | | C |
| ATOM | 3141 | C | PRO G | 480 | −7.662 | 16.056 | −28.548 | 1.00 | 52.36 | | C |
| ATOM | 3142 | O | PRO G | 480 | −8.804 | 15.721 | −28.860 | 1.00 | 64.15 | | O |
| ATOM | 3143 | N | LEU G | 481 | −6.698 | 15.183 | −28.281 | 1.00 | 62.90 | | N |
| ATOM | 3144 | CA | LEU G | 481 | −6.977 | 13.759 | −28.138 | 1.00 | 54.24 | | C |
| ATOM | 3145 | CB | LEU G | 481 | −6.143 | 12.944 | −29.128 | 1.00 | 20.00 | | C |
| ATOM | 3146 | CG | LEU G | 481 | −6.449 | 13.142 | −30.613 | 1.00 | 20.00 | | C |
| ATOM | 3147 | CD1 | LEU G | 481 | −5.475 | 12.348 | −31.471 | 1.00 | 20.00 | | C |
| ATOM | 3148 | CD2 | LEU G | 481 | −7.884 | 12.747 | −30.922 | 1.00 | 20.00 | | C |
| ATOM | 3149 | C | LEU G | 481 | −6.700 | 13.285 | −26.717 | 1.00 | 59.81 | | C |
| ATOM | 3150 | O | LEU G | 481 | −6.199 | 14.041 | −25.884 | 1.00 | 64.98 | | O |
| ATOM | 3151 | N | VAL G | 482 | −6.956 | 12.008 | −26.470 | 1.00 | 59.66 | | N |
| ATOM | 3152 | CA | VAL G | 482 | −6.588 | 11.404 | −25.203 | 1.00 | 49.67 | | C |
| ATOM | 3153 | CB | VAL G | 482 | −7.741 | 10.579 | −24.619 | 1.00 | 20.00 | | C |
| ATOM | 3154 | CG1 | VAL G | 482 | −7.390 | 10.116 | −23.220 | 1.00 | 20.00 | | C |
| ATOM | 3155 | CG2 | VAL G | 482 | −9.021 | 11.398 | −24.607 | 1.00 | 20.00 | | C |
| ATOM | 3156 | C | VAL G | 482 | −5.357 | 10.525 | −25.372 | 1.00 | 53.03 | | C |
| ATOM | 3157 | O | VAL G | 482 | −5.147 | 9.945 | −26.437 | 1.00 | 46.27 | | O |
| ATOM | 3158 | N | PHE G | 483 | −4.464 | 10.579 | −24.387 | 1.00 | 61.61 | | N |
| ATOM | 3159 | CA | PHE G | 483 | −3.282 | 9.723 | −24.370 | 1.00 | 65.03 | | C |
| ATOM | 3160 | CB | PHE G | 483 | −2.011 | 10.562 | −24.235 | 1.00 | 20.00 | | C |
| ATOM | 3161 | CG | PHE G | 483 | −1.788 | 11.500 | −25.385 | 1.00 | 20.00 | | C |
| ATOM | 3162 | CD1 | PHE G | 483 | −1.173 | 11.059 | −26.546 | 1.00 | 20.00 | | C |
| ATOM | 3163 | CE1 | PHE G | 483 | −1.039 | 11.897 | −27.632 | 1.00 | 20.00 | | C |
| ATOM | 3164 | CZ | PHE G | 483 | −1.577 | 13.170 | −27.589 | 1.00 | 20.00 | | C |
| ATOM | 3165 | CE2 | PHE G | 483 | −2.199 | 13.618 | −26.442 | 1.00 | 20.00 | | C |
| ATOM | 3166 | CD2 | PHE G | 483 | −2.315 | 12.781 | −25.355 | 1.00 | 20.00 | | C |
| ATOM | 3167 | C | PHE G | 483 | −3.358 | 8.663 | −23.275 | 1.00 | 71.00 | | C |
| ATOM | 3168 | O | PHE G | 483 | −2.688 | 8.776 | −22.248 | 1.00 | 100.44 | | O |
| ATOM | 3169 | N | PRO G | 484 | −4.338 | 7.759 | −23.403 | 1.00 | 54.44 | | N |
| ATOM | 3170 | CA | PRO G | 484 | −4.502 | 6.677 | −22.459 | 1.00 | 47.46 | | C |
| ATOM | 3171 | CB | PRO G | 484 | −5.761 | 7.096 | −21.701 | 1.00 | 20.00 | | C |
| ATOM | 3172 | CG | PRO G | 484 | −6.554 | 7.922 | −22.714 | 1.00 | 20.00 | | C |
| ATOM | 3173 | CD | PRO G | 484 | −5.649 | 8.206 | −23.894 | 1.00 | 20.00 | | C |
| ATOM | 3174 | C | PRO G | 484 | −4.778 | 5.378 | −23.204 | 1.00 | 41.56 | | C |
| ATOM | 3175 | O | PRO G | 484 | −5.689 | 5.321 | −24.029 | 1.00 | 46.53 | | O |
| ATOM | 3176 | N | SER G | 485 | −3.961 | 4.363 | −22.958 | 1.00 | 46.30 | | N |
| ATOM | 3177 | CA | SER G | 485 | −4.081 | 3.108 | −23.686 | 1.00 | 49.30 | | C |
| ATOM | 3178 | CB | SER G | 485 | −3.559 | 1.943 | −22.841 | 1.00 | 20.00 | | C |
| ATOM | 3179 | OG | SER G | 485 | −4.215 | 1.884 | −21.588 | 1.00 | 20.00 | | O |
| ATOM | 3180 | C | SER G | 485 | −5.521 | 2.856 | −24.147 | 1.00 | 41.24 | | C |
| ATOM | 3181 | O | SER G | 485 | −5.809 | 2.890 | −25.345 | 1.00 | 46.93 | | O |
| ATOM | 3182 | N | ASP G | 486 | −6.437 | 2.720 | −23.190 | 1.00 | 49.48 | | N |
| ATOM | 3183 | CA | ASP G | 486 | −7.816 | 2.321 | −23.485 | 1.00 | 37.44 | | C |
| ATOM | 3184 | CB | ASP G | 486 | −8.659 | 2.287 | −22.205 | 1.00 | 20.00 | | C |
| ATOM | 3185 | CG | ASP G | 486 | −8.296 | 1.125 | −21.289 | 1.00 | 20.00 | | C |
| ATOM | 3186 | OD1 | ASP G | 486 | −7.611 | 0.188 | −21.746 | 1.00 | 20.00 | | O |
| ATOM | 3187 | OD2 | ASP G | 486 | −8.637 | 1.184 | −20.089 | 1.00 | 20.00 | | O |
| ATOM | 3188 | C | ASP G | 486 | −8.447 | 3.271 | −24.491 | 1.00 | 45.66 | | C |
| ATOM | 3189 | O | ASP G | 486 | −9.005 | 2.844 | −25.501 | 1.00 | 39.86 | | O |
| ATOM | 3190 | N | GLU G | 487 | −8.317 | 4.566 | −24.225 | 1.00 | 49.20 | | N |
| ATOM | 3191 | CA | GLU G | 487 | −8.853 | 5.585 | −25.116 | 1.00 | 45.70 | | C |
| ATOM | 3192 | CB | GLU G | 487 | −8.656 | 6.982 | −24.525 | 1.00 | 20.00 | | C |
| ATOM | 3193 | CG | GLU G | 487 | −9.449 | 7.237 | −23.251 | 1.00 | 20.00 | | C |
| ATOM | 3194 | CD | GLU G | 487 | −9.144 | 8.590 | −22.635 | 1.00 | 20.00 | | C |
| ATOM | 3195 | OE1 | GLU G | 487 | −8.175 | 9.245 | −23.078 | 1.00 | 20.00 | | O |
| ATOM | 3196 | OE2 | GLU G | 487 | −9.865 | 8.988 | −21.695 | 1.00 | 20.00 | | O |
| ATOM | 3197 | C | GLU G | 487 | −8.222 | 5.504 | −26.501 | 1.00 | 41.48 | | C |
| ATOM | 3198 | O | GLU G | 487 | −8.914 | 5.623 | −27.512 | 1.00 | 48.92 | | O |
| ATOM | 3199 | N | PHE G | 488 | −6.910 | 5.306 | −26.545 | 1.00 | 42.16 | | N |
| ATOM | 3200 | CA | PHE G | 488 | −6.206 | 5.215 | −27.815 | 1.00 | 39.33 | | C |
| ATOM | 3201 | CB | PHE G | 488 | −4.701 | 5.086 | −27.590 | 1.00 | 20.00 | | C |
| ATOM | 3202 | CG | PHE G | 488 | −4.074 | 6.308 | −26.992 | 1.00 | 20.00 | | C |
| ATOM | 3203 | CD1 | PHE G | 488 | −3.910 | 7.454 | −27.747 | 1.00 | 20.00 | | C |
| ATOM | 3204 | CE1 | PHE G | 488 | −3.320 | 8.574 | −27.202 | 1.00 | 20.00 | | C |
| ATOM | 3205 | CZ | PHE G | 488 | −2.847 | 8.542 | −25.898 | 1.00 | 20.00 | | C |
| ATOM | 3206 | CE2 | PHE G | 488 | −3.006 | 7.404 | −25.136 | 1.00 | 20.00 | | C |
| ATOM | 3207 | CD2 | PHE G | 488 | −3.630 | 6.302 | −25.678 | 1.00 | 20.00 | | C |
| ATOM | 3208 | C | PHE G | 488 | −6.724 | 4.040 | −28.634 | 1.00 | 43.06 | | C |
| ATOM | 3209 | O | PHE G | 488 | −7.101 | 4.203 | −29.794 | 1.00 | 61.71 | | O |
| ATOM | 3210 | N | ASP G | 489 | −6.910 | 2.906 | −27.972 | 1.00 | 41.95 | | N |
| ATOM | 3211 | CA | ASP G | 489 | −7.330 | 1.695 | −28.658 | 1.00 | 42.75 | | C |
| ATOM | 3212 | CB | ASP G | 489 | −7.257 | 0.499 | −27.717 | 1.00 | 20.00 | | C |
| ATOM | 3213 | CG | ASP G | 489 | −5.838 | 0.116 | −27.389 | 1.00 | 20.00 | | C |
| ATOM | 3214 | OD1 | ASP G | 489 | −4.920 | 0.637 | −28.056 | 1.00 | 20.00 | | O |
| ATOM | 3215 | OD2 | ASP G | 489 | −5.636 | −0.633 | −26.411 | 1.00 | 20.00 | | O |
| ATOM | 3216 | C | ASP G | 489 | −8.733 | 1.825 | −29.233 | 1.00 | 45.61 | | C |
| ATOM | 3217 | O | ASP G | 489 | −9.066 | 1.181 | −30.226 | 1.00 | 54.39 | | O |
| ATOM | 3218 | N | ALA G | 490 | −9.568 | 2.622 | −28.575 | 1.00 | 42.89 | | N |
| ATOM | 3219 | CA | ALA G | 490 | −10.929 | 2.848 | −29.044 | 1.00 | 38.40 | | C |
| ATOM | 3220 | CB | ALA G | 490 | −11.760 | 3.516 | −27.963 | 1.00 | 20.00 | | C |
| ATOM | 3221 | C | ALA G | 490 | −10.932 | 3.687 | −30.315 | 1.00 | 39.67 | | C |
| ATOM | 3222 | O | ALA G | 490 | −11.651 | 3.386 | −31.267 | 1.00 | 56.80 | | O |
| ATOM | 3223 | N | SER G | 491 | −10.138 | 4.752 | −30.315 | 1.00 | 40.38 | | N |
| ATOM | 3224 | CA | SER G | 491 | −9.984 | 5.586 | −31.499 | 1.00 | 41.77 | | C |
| ATOM | 3225 | CB | SER G | 491 | −9.155 | 6.831 | −31.176 | 1.00 | 20.00 | | C |
| ATOM | 3226 | OG | SER G | 491 | −7.806 | 6.490 | −30.912 | 1.00 | 20.00 | | O |
| ATOM | 3227 | C | SER G | 491 | −9.333 | 4.801 | −32.631 | 1.00 | 39.01 | | C |
| ATOM | 3228 | O | SER G | 491 | −9.688 | 4.967 | −33.798 | 1.00 | 36.91 | | O |
| ATOM | 3229 | N | ILE G | 492 | −8.387 | 3.938 | −32.278 | 1.00 | 38.11 | | N |
| ATOM | 3230 | CA | ILE G | 492 | −7.741 | 3.077 | −33.259 | 1.00 | 41.21 | | C |
| ATOM | 3231 | CB | ILE G | 492 | −6.630 | 2.211 | −32.620 | 1.00 | 20.00 | | C |
| ATOM | 3232 | CG1 | ILE G | 492 | −5.507 | 3.092 | −32.061 | 1.00 | 20.00 | | C |
| ATOM | 3233 | CD1 | ILE G | 492 | −4.533 | 2.346 | −31.163 | 1.00 | 20.00 | | C |
| ATOM | 3234 | CG2 | ILE G | 492 | −6.067 | 1.219 | −33.634 | 1.00 | 20.00 | | C |
| ATOM | 3235 | C | ILE G | 492 | −8.773 | 2.170 | −33.916 | 1.00 | 39.22 | | C |
| ATOM | 3236 | O | ILE G | 492 | −8.778 | 2.008 | −35.135 | 1.00 | 47.54 | | O |
| ATOM | 3237 | N | SER G | 493 | −9.703 | 1.662 | −33.111 | 1.00 | 41.64 | | N |
| ATOM | 3238 | CA | SER G | 493 | −10.786 | 0.819 | −33.612 | 1.00 | 35.15 | | C |
| ATOM | 3239 | CB | SER G | 493 | −11.579 | 0.202 | −32.454 | 1.00 | 20.00 | | C |
| ATOM | 3240 | OG | SER G | 493 | −12.267 | 1.196 | −31.716 | 1.00 | 20.00 | | O |
| ATOM | 3241 | C | SER G | 493 | −11.716 | 1.614 | −34.523 | 1.00 | 47.90 | | C |
| ATOM | 3242 | O | SER G | 493 | −12.169 | 1.115 | −35.555 | 1.00 | 50.94 | | O |
| ATOM | 3243 | N | GLN G | 494 | −11.936 | 2.877 | −34.169 | 1.00 | 42.51 | | N |
| ATOM | 3244 | CA | GLN G | 494 | −12.789 | 3.766 | −34.955 | 1.00 | 41.96 | | C |
| ATOM | 3245 | CB | GLN G | 494 | −13.107 | 5.036 | −34.167 | 1.00 | 20.00 | | C |
| ATOM | 3246 | CG | GLN G | 494 | −13.991 | 4.813 | −32.955 | 1.00 | 20.00 | | C |
| ATOM | 3247 | CD | GLN G | 494 | −14.211 | 6.084 | −32.163 | 1.00 | 20.00 | | C |
| ATOM | 3248 | OE1 | GLN G | 494 | −13.406 | 7.013 | −32.223 | 1.00 | 20.00 | | O |
| ATOM | 3249 | NE2 | GLN G | 494 | −15.317 | 6.140 | −31.430 | 1.00 | 20.00 | | N |
| ATOM | 3250 | C | GLN G | 494 | −12.151 | 4.137 | −36.291 | 1.00 | 33.86 | | C |
| ATOM | 3251 | O | GLN G | 494 | −12.829 | 4.195 | −37.318 | 1.00 | 54.89 | | O |
| ATOM | 3252 | N | VAL G | 495 | −10.881 | 4.523 | −36.241 | 1.00 | 34.99 | | N |
| ATOM | 3253 | CA | VAL G | 495 | −10.108 | 4.772 | −37.451 | 1.00 | 38.42 | | C |
| ATOM | 3254 | CB | VAL G | 495 | −8.642 | 5.097 | −37.120 | 1.00 | 20.00 | | C |
| ATOM | 3255 | CG1 | VAL G | 495 | −7.840 | 5.297 | −38.394 | 1.00 | 20.00 | | C |
| ATOM | 3256 | CG2 | VAL G | 495 | −8.562 | 6.327 | −36.231 | 1.00 | 20.00 | | C |
| ATOM | 3257 | C | VAL G | 495 | −10.152 | 3.573 | −38.393 | 1.00 | 42.74 | | C |
| ATOM | 3258 | O | VAL G | 495 | −10.424 | 3.720 | −39.584 | 1.00 | 44.00 | | O |
| ATOM | 3259 | N | ASN G | 496 | −9.947 | 2.383 | −37.838 | 1.00 | 42.45 | | N |
| ATOM | 3260 | CA | ASN G | 496 | −9.967 | 1.147 | −38.617 | 1.00 | 43.01 | | C |
| ATOM | 3261 | CB | ASN G | 496 | −9.572 | −0.046 | −37.743 | 1.00 | 20.00 | | C |
| ATOM | 3262 | CG | ASN G | 496 | −8.118 | 0.000 | −37.319 | 1.00 | 20.00 | | C |
| ATOM | 3263 | OD1 | ASN G | 496 | −7.293 | 0.648 | −37.965 | 1.00 | 20.00 | | O |
| ATOM | 3264 | ND2 | ASN G | 496 | −7.796 | −0.684 | −36.226 | 1.00 | 20.00 | | N |
| ATOM | 3265 | C | ASN G | 496 | −11.305 | 0.875 | −39.310 | 1.00 | 43.13 | | C |
| ATOM | 3266 | O | ASN G | 496 | −11.334 | 0.405 | −40.448 | 1.00 | 54.76 | | O |
| ATOM | 3267 | N | GLU G | 497 | −12.407 | 1.099 | −38.598 | 1.00 | 42.08 | | N |
| ATOM | 3268 | CA | GLU G | 497 | −13.738 | 0.938 | −39.183 | 1.00 | 43.13 | | C |
| ATOM | 3269 | CB | GLU G | 497 | −14.827 | 1.144 | −38.127 | 1.00 | 20.00 | | C |
| ATOM | 3270 | CG | GLU G | 497 | −14.937 | 0.018 | −37.110 | 1.00 | 20.00 | | C |
| ATOM | 3271 | CD | GLU G | 497 | −15.946 | 0.316 | −36.017 | 1.00 | 20.00 | | C |
| ATOM | 3272 | OE1 | GLU G | 497 | −16.552 | 1.408 | −36.049 | 1.00 | 20.00 | | O |
| ATOM | 3273 | OE2 | GLU G | 497 | −16.177 | −0.566 | −35.164 | 1.00 | 20.00 | | O |
| ATOM | 3274 | C | GLU G | 497 | −13.937 | 1.915 | −40.337 | 1.00 | 49.77 | | C |
| ATOM | 3275 | O | GLU G | 497 | −14.552 | 1.580 | −41.349 | 1.00 | 42.88 | | O |
| ATOM | 3276 | N | LYS G | 498 | −13.367 | 3.108 | −40.195 | 1.00 | 41.82 | | N |
| ATOM | 3277 | CA | LYS G | 498 | −13.435 | 4.123 | −41.240 | 1.00 | 53.60 | | C |
| ATOM | 3278 | CB | LYS G | 498 | −12.968 | 5.479 | −40.705 | 1.00 | 20.00 | | C |
| ATOM | 3279 | CG | LYS G | 498 | −13.937 | 6.125 | −39.728 | 1.00 | 20.00 | | C |
| ATOM | 3280 | CD | LYS G | 498 | −13.439 | 7.484 | −39.269 | 1.00 | 20.00 | | C |
| ATOM | 3281 | CE | LYS G | 498 | −14.393 | 8.108 | −38.265 | 1.00 | 20.00 | | C |
| ATOM | 3282 | NZ | LYS G | 498 | −13.896 | 9.421 | −37.768 | 1.00 | 20.00 | | N |
| ATOM | 3283 | C | LYS G | 498 | −12.611 | 3.724 | −42.458 | 1.00 | 55.29 | | C |
| ATOM | 3284 | O | LYS G | 498 | −13.033 | 3.926 | −43.594 | 1.00 | 61.06 | | O |
| ATOM | 3285 | N | ILE G | 499 | −11.430 | 3.165 | −42.214 | 1.00 | 52.22 | | N |
| ATOM | 3286 | CA | ILE G | 499 | −10.609 | 2.604 | −43.284 | 1.00 | 48.26 | | C |
| ATOM | 3287 | CB | ILE G | 499 | −9.296 | 2.009 | −42.729 | 1.00 | 20.00 | | C |
| ATOM | 3288 | CG1 | ILE G | 499 | −8.446 | 3.101 | −42.072 | 1.00 | 20.00 | | C |
| ATOM | 3289 | CD1 | ILE G | 499 | −7.506 | 2.582 | −41.002 | 1.00 | 20.00 | | C |
| ATOM | 3290 | CG2 | ILE G | 499 | −8.516 | 1.288 | −43.825 | 1.00 | 20.00 | | C |
| ATOM | 3291 | C | ILE G | 499 | −11.376 | 1.515 | −44.031 | 1.00 | 49.07 | | C |
| ATOM | 3292 | O | ILE G | 499 | −11.282 | 1.398 | −45.253 | 1.00 | 58.45 | | O |
| ATOM | 3293 | N | ASN G | 500 | −12.183 | 0.764 | −43.288 | 1.00 | 51.02 | | N |
| ATOM | 3294 | CA | ASN G | 500 | −12.992 | −0.307 | −43.857 | 1.00 | 52.06 | | C |
| ATOM | 3295 | CB | ASN G | 500 | −13.500 | −1.238 | −42.748 | 1.00 | 20.00 | | C |
| ATOM | 3296 | CG | ASN G | 500 | −14.173 | −2.487 | −43.292 | 1.00 | 20.00 | | C |
| ATOM | 3297 | OD1 | ASN G | 500 | −13.795 | −3.005 | −44.342 | 1.00 | 20.00 | | O |
| ATOM | 3298 | ND2 | ASN G | 500 | −15.127 | −3.019 | −42.536 | 1.00 | 20.00 | | N |
| ATOM | 3299 | C | ASN G | 500 | −14.161 | 0.250 | −44.659 | 1.00 | 48.31 | | C |
| ATOM | 3300 | O | ASN G | 500 | −14.645 | −0.386 | −45.596 | 1.00 | 56.89 | | O |
| ATOM | 3301 | N | GLN G | 501 | −14.582 | 1.461 | −44.312 | 1.00 | 55.87 | | N |
| ATOM | 3302 | CA | GLN G | 501 | −15.607 | 2.160 | −45.074 | 1.00 | 53.55 | | C |
| ATOM | 3303 | CB | GLN G | 501 | −16.243 | 3.258 | −44.222 | 1.00 | 20.00 | | C |
| ATOM | 3304 | CG | GLN G | 501 | −17.012 | 2.741 | −43.015 | 1.00 | 20.00 | | C |
| ATOM | 3305 | CD | GLN G | 501 | −17.674 | 3.857 | −42.226 | 1.00 | 20.00 | | C |
| ATOM | 3306 | OE1 | GLN G | 501 | −17.178 | 4.985 | −42.189 | 1.00 | 20.00 | | O |
| ATOM | 3307 | NE2 | GLN G | 501 | −18.772 | 3.536 | −41.553 | 1.00 | 20.00 | | N |
| ATOM | 3308 | C | GLN G | 501 | −15.030 | 2.751 | −46.357 | 1.00 | 45.67 | | C |
| ATOM | 3309 | O | GLN G | 501 | −15.627 | 2.633 | −47.429 | 1.00 | 58.31 | | O |
| ATOM | 3310 | N | SER G | 502 | −13.824 | 3.303 | −46.258 | 1.00 | 43.44 | | N |
| ATOM | 3311 | CA | SER G | 502 | −13.117 | 3.801 | −47.432 | 1.00 | 45.40 | | C |
| ATOM | 3312 | CB | SER G | 502 | −11.789 | 4.468 | −47.045 | 1.00 | 20.00 | | C |
| ATOM | 3313 | OG | SER G | 502 | −10.928 | 3.572 | −46.361 | 1.00 | 20.00 | | O |
| ATOM | 3314 | C | SER G | 502 | −12.891 | 2.677 | −48.436 | 1.00 | 55.15 | | C |
| ATOM | 3315 | O | SER G | 502 | −13.234 | 2.819 | −49.610 | 1.00 | 52.57 | | O |
| ATOM | 3316 | N | LEU G | 503 | −12.552 | 1.495 | −47.922 | 1.00 | 55.35 | | N |
| ATOM | 3317 | CA | LEU G | 503 | −12.227 | 0.349 | −48.768 | 1.00 | 52.12 | | C |
| ATOM | 3318 | CB | LEU G | 503 | −11.591 | −0.778 | −47.943 | 1.00 | 20.00 | | C |
| ATOM | 3319 | CG | LEU G | 503 | −10.187 | −0.535 | −47.373 | 1.00 | 20.00 | | C |
| ATOM | 3320 | CD1 | LEU G | 503 | −9.810 | −1.609 | −46.357 | 1.00 | 20.00 | | C |
| ATOM | 3321 | CD2 | LEU G | 503 | −9.144 | −0.458 | −48.481 | 1.00 | 20.00 | | C |
| ATOM | 3322 | C | LEU G | 503 | −13.471 | −0.159 | −49.489 | 1.00 | 55.74 | | C |
| ATOM | 3323 | O | LEU G | 503 | −13.402 | −0.586 | −50.642 | 1.00 | 57.75 | | O |
| ATOM | 3324 | N | ALA G | 504 | −14.618 | −0.046 | −48.826 | 1.00 | 57.22 | | N |
| ATOM | 3325 | CA | ALA G | 504 | −15.893 | −0.410 | −49.436 | 1.00 | 59.12 | | C |
| ATOM | 3326 | CB | ALA G | 504 | −16.981 | −0.532 | −48.377 | 1.00 | 20.00 | | C |
| ATOM | 3327 | C | ALA G | 504 | −16.293 | 0.605 | −50.499 | 1.00 | 58.10 | | C |
| ATOM | 3328 | O | ALA G | 504 | −16.773 | 0.241 | −51.573 | 1.00 | 63.64 | | O |
| ATOM | 3329 | N | PHE G | 505 | −16.075 | 1.881 | −50.198 | 1.00 | 58.17 | | N |
| ATOM | 3330 | CA | PHE G | 505 | −16.303 | 2.940 | −51.171 | 1.00 | 56.56 | | C |
| ATOM | 3331 | CB | PHE G | 505 | −15.991 | 4.309 | −50.564 | 1.00 | 20.00 | | C |
| ATOM | 3332 | CG | PHE G | 505 | −16.870 | 4.665 | −49.405 | 1.00 | 20.00 | | C |
| ATOM | 3333 | CD1 | PHE G | 505 | −18.185 | 5.035 | −49.615 | 1.00 | 20.00 | | C |
| ATOM | 3334 | CE1 | PHE G | 505 | −19.045 | 5.205 | −48.555 | 1.00 | 20.00 | | C |
| ATOM | 3335 | CZ | PHE G | 505 | −18.617 | 4.934 | −47.269 | 1.00 | 20.00 | | C |
| ATOM | 3336 | CE2 | PHE G | 505 | −17.314 | 4.545 | −47.047 | 1.00 | 20.00 | | C |
| ATOM | 3337 | CD2 | PHE G | 505 | −16.446 | 4.423 | −48.109 | 1.00 | 20.00 | | C |
| ATOM | 3338 | C | PHE G | 505 | −15.462 | 2.715 | −52.416 | 1.00 | 60.12 | | C |
| ATOM | 3339 | O | PHE G | 505 | −15.922 | 2.939 | −53.535 | 1.00 | 65.46 | | O |
| ATOM | 3340 | N | ILE G | 506 | −14.235 | 2.242 | −52.213 | 1.00 | 56.10 | | N |
| ATOM | 3341 | CA | ILE G | 506 | −13.325 | 1.960 | −53.320 | 1.00 | 57.32 | | C |
| ATOM | 3342 | CB | ILE G | 506 | −11.911 | 1.590 | −52.825 | 1.00 | 20.00 | | C |
| ATOM | 3343 | CG1 | ILE G | 506 | −11.167 | 2.839 | −52.352 | 1.00 | 20.00 | | C |
| ATOM | 3344 | CD1 | ILE G | 506 | −10.010 | 2.533 | −51.425 | 1.00 | 20.00 | | C |
| ATOM | 3345 | CG2 | ILE G | 506 | −11.126 | 0.882 | −53.925 | 1.00 | 20.00 | | C |
| ATOM | 3346 | C | ILE G | 506 | −13.845 | 0.812 | −54.170 | 1.00 | 67.33 | | C |
| ATOM | 3347 | O | ILE G | 506 | −13.674 | 0.803 | −55.390 | 1.00 | 76.99 | | O |
| ATOM | 3348 | N | ARG G | 507 | −14.325 | −0.230 | −53.503 | 1.00 | 67.87 | | N |
| ATOM | 3349 | CA | ARG G | 507 | −14.920 | −1.360 | −54.195 | 1.00 | 62.11 | | C |
| ATOM | 3350 | CB | ARG G | 507 | −15.328 | −2.440 | −53.196 | 1.00 | 20.00 | | C |
| ATOM | 3351 | CG | ARG G | 507 | −14.151 | −3.082 | −52.491 | 1.00 | 20.00 | | C |
| ATOM | 3352 | CD | ARG G | 507 | −14.592 | −4.218 | −51.585 | 1.00 | 20.00 | | C |
| ATOM | 3353 | NE | ARG G | 507 | −13.453 | −4.836 | −50.911 | 1.00 | 20.00 | | N |
| ATOM | 3354 | CZ | ARG G | 507 | −13.555 | −5.749 | −49.950 | 1.00 | 20.00 | | C |
| ATOM | 3355 | NH1 | ARG G | 507 | −14.748 | −6.221 | −49.609 | 1.00 | 20.00 | | N |
| ATOM | 3356 | NH2 | ARG G | 507 | −12.459 | −6.278 | −49.422 | 1.00 | 20.00 | | N |
| ATOM | 3357 | C | ARG G | 507 | −16.119 | −0.920 | −55.026 | 1.00 | 67.30 | | C |
| ATOM | 3358 | O | ARG G | 507 | −16.131 | −1.079 | −56.247 | 1.00 | 67.02 | | O |
| ATOM | 3359 | N | LYS G | 508 | −17.080 | −0.274 | −54.375 | 1.00 | 66.83 | | N |
| ATOM | 3360 | CA | LYS G | 508 | −18.220 | 0.295 | −55.078 | 1.00 | 68.12 | | C |
| ATOM | 3361 | CB | LYS G | 508 | −19.052 | 1.156 | −54.133 | 1.00 | 20.00 | | C |
| ATOM | 3362 | CG | LYS G | 508 | −19.813 | 0.362 | −53.096 | 1.00 | 20.00 | | C |
| ATOM | 3363 | CD | LYS G | 508 | −20.707 | 1.269 | −52.270 | 1.00 | 20.00 | | C |
| ATOM | 3364 | CE | LYS G | 508 | −21.508 | 0.479 | −51.248 | 1.00 | 20.00 | | C |
| ATOM | 3365 | NZ | LYS G | 508 | −22.370 | 1.366 | −50.421 | 1.00 | 20.00 | | N |
| ATOM | 3366 | C | LYS G | 508 | −17.758 | 1.131 | −56.261 | 1.00 | 65.22 | | C |
| ATOM | 3367 | O | LYS G | 508 | −18.149 | 0.880 | −57.402 | 1.00 | 72.34 | | O |
| ATOM | 3368 | N | SER G | 509 | −16.859 | 2.071 | −55.990 | 1.00 | 64.87 | | N |
| ATOM | 3369 | CA | SER G | 509 | −16.316 | 2.939 | −57.028 | 1.00 | 56.47 | | C |
| ATOM | 3370 | CB | SER G | 509 | −15.268 | 3.896 | −56.442 | 1.00 | 20.00 | | C |
| ATOM | 3371 | OG | SER G | 509 | −14.170 | 3.190 | −55.888 | 1.00 | 20.00 | | O |
| ATOM | 3372 | C | SER G | 509 | −15.736 | 2.147 | −58.205 | 1.00 | 56.96 | | C |
| ATOM | 3373 | O | SER G | 509 | −16.064 | 2.412 | −59.363 | 1.00 | 65.57 | | O |
| ATOM | 3374 | N | ASP G | 510 | −14.906 | 1.154 | −57.902 | 1.00 | 64.34 | | N |
| ATOM | 3375 | CA | ASP G | 510 | −14.282 | 0.340 | −58.939 | 1.00 | 64.83 | | C |
| ATOM | 3376 | CB | ASP G | 510 | −13.338 | −0.690 | −58.316 | 1.00 | 20.00 | | C |
| ATOM | 3377 | CG | ASP G | 510 | −12.073 | −0.063 | −57.768 | 1.00 | 20.00 | | C |
| ATOM | 3378 | OD1 | ASP G | 510 | −11.789 | 1.104 | −58.112 | 1.00 | 20.00 | | O |
| ATOM | 3379 | OD2 | ASP G | 510 | −11.361 | −0.735 | −56.993 | 1.00 | 20.00 | | O |
| ATOM | 3380 | C | ASP G | 510 | −15.339 | −0.362 | −59.782 | 1.00 | 62.11 | | C |
| ATOM | 3381 | O | ASP G | 510 | −15.346 | −0.251 | −61.008 | 1.00 | 67.61 | | O |
| ATOM | 3382 | N | GLU G | 511 | −16.244 | −1.067 | −59.112 | 1.00 | 64.95 | | N |
| ATOM | 3383 | CA | GLU G | 511 | −17.319 | −1.774 | −59.791 | 1.00 | 60.64 | | C |
| ATOM | 3384 | CB | GLU G | 511 | −18.359 | −2.259 | −58.780 | 1.00 | 20.00 | | C |
| ATOM | 3385 | CG | GLU G | 511 | −17.818 | −3.293 | −57.801 | 1.00 | 20.00 | | C |
| ATOM | 3386 | CD | GLU G | 511 | −18.840 | −3.723 | −56.760 | 1.00 | 20.00 | | C |
| ATOM | 3387 | OE1 | GLU G | 511 | −19.857 | −3.017 | −56.585 | 1.00 | 20.00 | | O |
| ATOM | 3388 | OE2 | GLU G | 511 | −18.603 | −4.746 | −56.083 | 1.00 | 20.00 | | O |
| ATOM | 3389 | C | GLU G | 511 | −17.970 | −0.888 | −60.842 | 1.00 | 59.91 | | C |
| ATOM | 3390 | O | GLU G | 511 | −18.090 | −1.278 | −62.003 | 1.00 | 55.45 | | O |
| ATOM | 3391 | N | LEU G | 512 | −18.270 | 0.349 | −60.460 | 1.00 | 44.35 | | N |
| ATOM | 3392 | CA | LEU G | 512 | −18.978 | 1.259 | −61.346 | 1.00 | 49.60 | | C |
| ATOM | 3393 | CB | LEU G | 512 | −19.424 | 2.511 | −60.590 | 1.00 | 20.00 | | C |
| ATOM | 3394 | CG | LEU G | 512 | −20.377 | 2.256 | −59.416 | 1.00 | 20.00 | | C |
| ATOM | 3395 | CD1 | LEU G | 512 | −20.747 | 3.550 | −58.696 | 1.00 | 20.00 | | C |
| ATOM | 3396 | CD2 | LEU G | 512 | −21.624 | 1.499 | −59.858 | 1.00 | 20.00 | | C |
| ATOM | 3397 | C | LEU G | 512 | −18.128 | 1.626 | −62.557 | 1.00 | 58.00 | | C |
| ATOM | 3398 | O | LEU G | 512 | −18.542 | 1.415 | −63.696 | 1.00 | 55.11 | | O |
| ATOM | 3399 | N | LEU G | 513 | −16.883 | 2.015 | −62.305 | 1.00 | 57.91 | | N |
| ATOM | 3400 | CA | LEU G | 513 | −15.963 | 2.384 | −63.378 | 1.00 | 57.49 | | C |
| ATOM | 3401 | CB | LEU G | 513 | −14.609 | 2.810 | −62.801 | 1.00 | 20.00 | | C |
| ATOM | 3402 | CG | LEU G | 513 | −14.558 | 4.136 | −62.038 | 1.00 | 20.00 | | C |
| ATOM | 3403 | CD1 | LEU G | 513 | −13.242 | 4.263 | −61.282 | 1.00 | 20.00 | | C |
| ATOM | 3404 | CD2 | LEU G | 513 | −14.743 | 5.302 | −62.996 | 1.00 | 20.00 | | C |
| ATOM | 3405 | C | LEU G | 513 | −15.775 | 1.231 | −64.362 | 1.00 | 58.78 | | C |
| ATOM | 3406 | O | LEU G | 513 | −15.707 | 1.437 | −65.574 | 1.00 | 59.88 | | O |
| ATOM | 3407 | N | HIS G | 514 | −15.708 | 0.015 | −63.829 | 1.00 | 60.07 | | N |
| ATOM | 3408 | CA | HIS G | 514 | −15.540 | −1.175 | −64.652 | 1.00 | 62.82 | | C |
| ATOM | 3409 | CB | HIS G | 514 | −15.189 | −2.383 | −63.782 | 1.00 | 20.00 | | C |
| ATOM | 3410 | CG | HIS G | 514 | −13.924 | −2.217 | −63.001 | 1.00 | 20.00 | | C |
| ATOM | 3411 | ND1 | HIS G | 514 | −12.683 | −2.154 | −63.598 | 1.00 | 20.00 | | N |
| ATOM | 3412 | CE1 | HIS G | 514 | −11.757 | −2.005 | −62.668 | 1.00 | 20.00 | | C |
| ATOM | 3413 | NE2 | HIS G | 514 | −12.351 | −1.975 | −61.489 | 1.00 | 20.00 | | N |
| ATOM | 3414 | CD2 | HIS G | 514 | −13.706 | −2.113 | −61.669 | 1.00 | 20.00 | | C |
| ATOM | 3415 | C | HIS G | 514 | −16.796 | −1.465 | −65.463 | 1.00 | 65.10 | | C |
| ATOM | 3416 | O | HIS G | 514 | −16.745 | −2.170 | −66.470 | 1.00 | 66.73 | | O |
| ATOM | 3417 | N | ASN G | 515 | −17.926 | −0.941 | −65.000 | 1.00 | 63.45 | | N |
| ATOM | 3418 | CA | ASN G | 515 | −19.179 | −1.062 | −65.732 | 1.00 | 56.53 | | C |
| ATOM | 3419 | CB | ASN G | 515 | −20.370 | −0.925 | −64.783 | 1.00 | 20.00 | | C |
| ATOM | 3420 | CG | ASN G | 515 | −20.503 | −2.107 | −63.845 | 1.00 | 20.00 | | C |
| ATOM | 3421 | OD1 | ASN G | 515 | −19.979 | −3.190 | −64.113 | 1.00 | 20.00 | | O |
| ATOM | 3422 | ND2 | ASN G | 515 | −21.206 | −1.906 | −62.734 | 1.00 | 20.00 | | N |
| ATOM | 3423 | C | ASN G | 515 | −19.289 | −0.043 | −66.860 | 1.00 | 60.06 | | C |
| ATOM | 3424 | O | ASN G | 515 | −19.825 | −0.335 | −67.928 | 1.00 | 66.48 | | O |
| ATOM | 3425 | N | VAL G | 516 | −18.818 | 1.168 | −66.598 | 1.00 | 54.29 | | N |
| ATOM | 3426 | CA | VAL G | 516 | −18.783 | 2.200 | −67.620 | 1.00 | 52.70 | | C |
| ATOM | 3427 | CB | VAL G | 516 | −18.233 | 3.518 | −67.056 | 1.00 | 20.00 | | C |
| ATOM | 3428 | CG1 | VAL G | 516 | −18.151 | 4.566 | −68.153 | 1.00 | 20.00 | | C |
| ATOM | 3429 | CG2 | VAL G | 516 | −19.098 | 4.003 | −65.904 | 1.00 | 20.00 | | C |
| ATOM | 3430 | C | VAL G | 516 | −17.910 | 1.753 | −68.784 | 1.00 | 60.10 | | C |
| ATOM | 3431 | O | VAL G | 516 | −18.340 | 1.765 | −69.937 | 1.00 | 61.55 | | O |
| ATOM | 3432 | N | ASN G | 517 | −16.733 | 1.232 | −68.455 | 1.00 | 69.10 | | N |
| ATOM | 3433 | CA | ASN G | 517 | −15.793 | 0.770 | −69.466 | 1.00 | 67.75 | | C |
| ATOM | 3434 | CB | ASN G | 517 | −14.564 | 0.137 | −68.807 | 1.00 | 20.00 | | C |
| ATOM | 3435 | CG | ASN G | 517 | −13.665 | 1.163 | −68.146 | 1.00 | 20.00 | | C |
| ATOM | 3436 | OD1 | ASN G | 517 | −13.646 | 2.330 | −68.534 | 1.00 | 20.00 | | O |
| ATOM | 3437 | ND2 | ASN G | 517 | −12.873 | 0.718 | −67.177 | 1.00 | 20.00 | | N |
| ATOM | 3438 | C | ASN G | 517 | −16.439 | −0.209 | −70.444 | 1.00 | 71.17 | | C |
| ATOM | 3439 | O | ASN G | 517 | −16.188 | −0.154 | −71.649 | 1.00 | 66.42 | | O |
| ATOM | 3440 | N | ALA G | 518 | −17.351 | −1.033 | −69.935 | 1.00 | 72.11 | | N |
| ATOM | 3441 | CA | ALA G | 518 | −18.026 | −2.024 | −70.764 | 1.00 | 74.82 | | C |
| ATOM | 3442 | CB | ALA G | 518 | −18.558 | −3.164 | −69.912 | 1.00 | 20.00 | | C |
| ATOM | 3443 | C | ALA G | 518 | −19.145 | −1.399 | −71.593 | 1.00 | 70.11 | | C |
| ATOM | 3444 | O | ALA G | 518 | −19.363 | −1.789 | −72.739 | 1.00 | 65.18 | | O |
| ATOM | 3445 | N | GLY G | 519 | −19.865 | −0.448 | −71.006 | 1.00 | 56.07 | | N |
| ATOM | 3446 | CA | GLY G | 519 | −20.825 | 0.357 | −71.760 | 1.00 | 53.01 | | C |
| ATOM | 3447 | C | GLY G | 519 | −20.156 | 1.166 | −72.856 | 1.00 | 63.58 | | C |
| ATOM | 3448 | O | GLY G | 519 | −20.684 | 1.295 | −73.962 | 1.00 | 69.35 | | O |
| ATOM | 3449 | N | LYS G | 520 | −18.950 | 1.647 | −72.561 | 1.00 | 58.33 | | N |
| ATOM | 3450 | CA | LYS G | 520 | −18.122 | 2.361 | −73.533 | 1.00 | 53.44 | | C |
| ATOM | 3451 | CB | LYS G | 520 | −16.848 | 2.877 | −72.865 | 1.00 | 20.00 | | C |
| ATOM | 3452 | CG | LYS G | 520 | −17.081 | 3.958 | −71.831 | 1.00 | 20.00 | | C |
| ATOM | 3453 | CD | LYS G | 520 | −15.781 | 4.341 | −71.152 | 1.00 | 20.00 | | C |
| ATOM | 3454 | CE | LYS G | 520 | −15.984 | 5.482 | −70.173 | 1.00 | 20.00 | | C |
| ATOM | 3455 | NZ | LYS G | 520 | −14.753 | 5.730 | −69.376 | 1.00 | 20.00 | | N |
| ATOM | 3456 | C | LYS G | 520 | −17.746 | 1.492 | −74.731 | 1.00 | 58.24 | | C |
| ATOM | 3457 | O | LYS G | 520 | −17.445 | 2.006 | −75.810 | 1.00 | 48.60 | | O |
| ATOM | 3458 | N | SER G | 521 | −17.583 | 0.198 | −74.478 | 1.00 | 67.19 | | N |
| ATOM | 3459 | CA | SER G | 521 | −17.366 | −0.776 | −75.542 | 1.00 | 60.42 | | C |
| ATOM | 3460 | CB | SER G | 521 | −16.953 | −2.131 | −74.959 | 1.00 | 20.00 | | C |
| ATOM | 3461 | OG | SER G | 521 | −18.028 | −2.751 | −74.272 | 1.00 | 20.00 | | O |
| ATOM | 3462 | C | SER G | 521 | −18.588 | −0.942 | −76.444 | 1.00 | 54.19 | | C |
| ATOM | 3463 | O | SER G | 521 | −18.456 | −1.004 | −77.664 | 1.00 | 65.36 | | O |
| ATOM | 3464 | N | THR G | 522 | −19.761 | −1.120 | −75.840 | 1.00 | 50.09 | | N |
| ATOM | 3465 | CA | THR G | 522 | −21.001 | −1.210 | −76.608 | 1.00 | 57.59 | | C |
| ATOM | 3466 | CB | THR G | 522 | −22.235 | −1.431 | −75.705 | 1.00 | 20.00 | | C |
| ATOM | 3467 | OG1 | THR G | 522 | −22.276 | −0.422 | −74.689 | 1.00 | 20.00 | | O |
| ATOM | 3468 | CG2 | THR G | 522 | −22.196 | −2.805 | −75.058 | 1.00 | 20.00 | | C |
| ATOM | 3469 | C | THR G | 522 | −21.206 | 0.063 | −77.423 | 1.00 | 59.16 | | C |
| ATOM | 3470 | O | THR G | 522 | −21.384 | 0.010 | −78.640 | 1.00 | 65.90 | | O |
| ATOM | 3471 | N | THR G | 523 | −21.024 | 1.205 | −76.768 | 1.00 | 52.59 | | N |
| ATOM | 3472 | CA | THR G | 523 | −21.234 | 2.498 | −77.404 | 1.00 | 42.19 | | C |
| ATOM | 3473 | CB | THR G | 523 | −20.961 | 3.638 | −76.423 | 1.00 | 20.00 | | C |
| ATOM | 3474 | OG1 | THR G | 523 | −19.578 | 3.622 | −76.049 | 1.00 | 20.00 | | O |
| ATOM | 3475 | CG2 | THR G | 523 | −21.819 | 3.467 | −75.180 | 1.00 | 20.00 | | C |
| ATOM | 3476 | C | THR G | 523 | −20.352 | 2.676 | −78.636 | 1.00 | 48.90 | | C |
| ATOM | 3477 | O | THR G | 523 | −20.786 | 3.238 | −79.641 | 1.00 | 63.19 | | O |
| ATOM | 3478 | N | ASN G | 524 | −19.113 | 2.197 | −78.553 | 1.00 | 52.51 | | N |
| ATOM | 3479 | CA | ASN G | 524 | −18.206 | 2.200 | −79.701 | 1.00 | 57.54 | | C |
| ATOM | 3480 | CB | ASN G | 524 | −16.783 | 1.822 | −79.276 | 1.00 | 20.00 | | C |
| ATOM | 3481 | CG | ASN G | 524 | −16.110 | 2.908 | −78.457 | 1.00 | 20.00 | | C |
| ATOM | 3482 | OD1 | ASN G | 524 | −16.515 | 4.069 | −78.492 | 1.00 | 20.00 | | O |
| ATOM | 3483 | ND2 | ASN G | 524 | −15.087 | 2.529 | −77.699 | 1.00 | 20.00 | | N |
| ATOM | 3484 | C | ASN G | 524 | −18.669 | 1.296 | −80.843 | 1.00 | 54.12 | | C |
| ATOM | 3485 | O | ASN G | 524 | −18.530 | 1.652 | −82.013 | 1.00 | 61.31 | | O |
| ATOM | 3486 | N | SER G | 525 | −19.149 | 0.102 | −80.502 | 1.00 | 48.97 | | N |
| ATOM | 3487 | CA | SER G | 525 | −19.658 | −0.841 | −81.499 | 1.00 | 64.81 | | C |
| ATOM | 3488 | CB | SER G | 525 | −20.077 | −2.157 | −80.838 | 1.00 | 61.22 | | C |
| ATOM | 3489 | OG | SER G | 525 | −18.947 | −2.929 | −80.475 | 1.00 | 85.90 | | O |
| ATOM | 3490 | C | SER G | 525 | −20.840 | −0.249 | −82.254 | 1.00 | 69.67 | | C |
| ATOM | 3491 | O | SER G | 525 | −21.093 | −0.600 | −83.406 | 1.00 | 72.60 | | O |
| ATOM | 3492 | N | LYS G | 526 | −21.592 | 0.610 | −81.577 | 1.00 | 66.06 | | N |
| ATOM | 3493 | CA | LYS G | 526 | −22.723 | 1.280 | −82.197 | 1.00 | 63.67 | | C |
| ATOM | 3494 | CB | LYS G | 526 | −23.675 | 1.815 | −81.127 | 1.00 | 70.55 | | C |
| ATOM | 3495 | CG | LYS G | 526 | −24.473 | 0.741 | −80.407 | 1.00 | 75.29 | | C |
| ATOM | 3496 | CD | LYS G | 526 | −25.171 | 1.313 | −79.183 | 1.00 | 85.03 | | C |
| ATOM | 3497 | CE | LYS G | 526 | −25.776 | 0.219 | −78.325 | 1.00 | 82.65 | | C |
| ATOM | 3498 | NZ | LYS G | 526 | −26.303 | 0.770 | −77.048 | 1.00 | 73.89 | | N |
| ATOM | 3499 | C | LYS G | 526 | −22.259 | 2.417 | −83.103 | 1.00 | 55.36 | | C |
| ATOM | 3500 | O | LYS G | 526 | −22.797 | 2.611 | −84.191 | 1.00 | 70.69 | | O |
| ATOM | 3501 | N | ILE G | 527 | −21.275 | 3.181 | −82.638 | 1.00 | 57.52 | | N |
| ATOM | 3502 | CA | ILE G | 527 | −20.718 | 4.273 | −83.431 | 1.00 | 58.48 | | C |
| ATOM | 3503 | CB | ILE G | 527 | −19.545 | 4.966 | −82.712 | 1.00 | 57.00 | | C |
| ATOM | 3504 | CG1 | ILE G | 527 | −20.021 | 5.620 | −81.415 | 1.00 | 49.07 | | C |
| ATOM | 3505 | CD1 | ILE G | 527 | −20.228 | 7.116 | −81.534 | 1.00 | 46.18 | | C |
| ATOM | 3506 | CG2 | ILE G | 527 | −18.902 | 6.004 | −83.621 | 1.00 | 52.63 | | C |
| ATOM | 3507 | C | ILE G | 527 | −20.224 | 3.758 | −84.774 | 1.00 | 65.20 | | C |
| ATOM | 3508 | O | ILE G | 527 | −20.455 | 4.380 | −85.810 | 1.00 | 66.48 | | O |
| ATOM | 3509 | N | TYR G | 528 | −19.487 | 2.652 | −84.740 | 1.00 | 69.78 | | N |
| ATOM | 3510 | CA | TYR G | 528 | −18.985 | 2.034 | −85.961 | 1.00 | 72.52 | | C |
| ATOM | 3511 | CB | TYR G | 528 | −18.268 | 0.712 | −85.657 | 1.00 | 73.16 | | C |
| ATOM | 3512 | CG | TYR G | 528 | −17.130 | 0.810 | −84.658 | 1.00 | 76.94 | | C |
| ATOM | 3513 | CD1 | TYR G | 528 | −16.667 | −0.323 | −83.994 | 1.00 | 70.95 | | C |
| ATOM | 3514 | CE1 | TYR G | 528 | −15.628 | −0.245 | −83.077 | 1.00 | 67.73 | | C |
| ATOM | 3515 | CZ | TYR G | 528 | −15.064 | 0.981 | −82.789 | 1.00 | 71.89 | | C |
| ATOM | 3516 | OH | TYR G | 528 | −14.046 | 1.065 | −81.866 | 1.00 | 75.44 | | O |
| ATOM | 3517 | CE2 | TYR G | 528 | −15.505 | 2.121 | −83.433 | 1.00 | 77.03 | | C |
| ATOM | 3518 | CD2 | TYR G | 528 | −16.523 | 2.029 | −84.373 | 1.00 | 77.84 | | C |
| ATOM | 3519 | C | TYR G | 528 | −20.130 | 1.796 | −86.949 | 1.00 | 71.63 | | C |
| ATOM | 3520 | O | TYR G | 528 | −20.150 | 2.377 | −88.036 | 1.00 | 84.20 | | O |
| ATOM | 3521 | N | HIS G | 529 | −21.116 | 1.002 | −86.534 | 1.00 | 67.69 | | N |
| ATOM | 3522 | CA | HIS G | 529 | −22.239 | 0.633 | −87.398 | 1.00 | 60.28 | | C |
| ATOM | 3523 | CB | HIS G | 529 | −23.215 | −0.283 | −86.659 | 1.00 | 67.84 | | C |
| ATOM | 3524 | CG | HIS G | 529 | −22.668 | −1.645 | −86.376 | 1.00 | 88.54 | | C |
| ATOM | 3525 | ND1 | HIS G | 529 | −23.472 | −2.756 | −86.236 | 1.00 | 93.34 | | N |
| ATOM | 3526 | CE1 | HIS G | 529 | −22.725 | −3.802 | −85.933 | 1.00 | 86.23 | | C |
| ATOM | 3527 | NE2 | HIS G | 529 | −21.465 | −3.410 | −85.871 | 1.00 | 91.95 | | N |
| ATOM | 3528 | CD2 | HIS G | 529 | −21.401 | −2.067 | −86.151 | 1.00 | 93.77 | | C |
| ATOM | 3529 | C | HIS G | 529 | −22.987 | 1.845 | −87.944 | 1.00 | 63.30 | | C |
| ATOM | 3530 | O | HIS G | 529 | −23.453 | 1.828 | −89.083 | 1.00 | 61.02 | | O |
| ATOM | 3531 | N | ILE G | 530 | −23.209 | 2.842 | −87.091 | 1.00 | 59.79 | | N |
| ATOM | 3532 | CA | ILE G | 530 | −23.752 | 4.121 | −87.543 | 1.00 | 45.70 | | C |
| ATOM | 3533 | CB | ILE G | 530 | −23.965 | 5.095 | −86.374 | 1.00 | 40.57 | | C |
| ATOM | 3534 | CG1 | ILE G | 530 | −25.130 | 4.627 | −85.499 | 1.00 | 42.86 | | C |
| ATOM | 3535 | CD1 | ILE G | 530 | −25.537 | 5.624 | −84.437 | 1.00 | 51.01 | | C |
| ATOM | 3536 | CG2 | ILE G | 530 | −24.224 | 6.501 | −86.901 | 1.00 | 35.31 | | C |
| ATOM | 3537 | C | ILE G | 530 | −22.822 | 4.775 | −88.557 | 1.00 | 57.52 | | C |
| ATOM | 3538 | O | ILE G | 530 | −23.260 | 5.253 | −89.604 | 1.00 | 71.18 | | O |
| ATOM | 3539 | N | GLU G | 531 | −21.534 | 4.787 | −88.234 | 1.00 | 67.55 | | N |
| ATOM | 3540 | CA | GLU G | 531 | −20.508 | 5.305 | −89.129 | 1.00 | 69.79 | | C |
| ATOM | 3541 | CB | GLU G | 531 | −19.126 | 5.105 | −88.505 | 1.00 | 84.02 | | C |
| ATOM | 3542 | CG | GLU G | 531 | −18.143 | 6.228 | −88.780 | 1.00 | 104.22 | | C |
| ATOM | 3543 | CD | GLU G | 531 | −16.823 | 6.033 | −88.058 | 1.00 | 116.08 | | C |
| ATOM | 3544 | OE1 | GLU G | 531 | −16.849 | 5.706 | −86.853 | 1.00 | 118.71 | | O |
| ATOM | 3545 | OE2 | GLU G | 531 | −15.762 | 6.211 | −88.693 | 1.00 | 119.69 | | O |
| ATOM | 3546 | C | GLU G | 531 | −20.567 | 4.624 | −90.495 | 1.00 | 64.87 | | C |
| ATOM | 3547 | O | GLU G | 531 | −20.720 | 5.285 | −91.522 | 1.00 | 55.58 | | O |
| ATOM | 3548 | N | ASN G | 532 | −20.409 | 3.304 | −90.499 | 1.00 | 65.70 | | N |
| ATOM | 3549 | CA | ASN G | 532 | −20.502 | 2.516 | −91.724 | 1.00 | 64.16 | | C |
| ATOM | 3550 | CB | ASN G | 532 | −20.351 | 1.026 | −91.402 | 1.00 | 60.58 | | C |
| ATOM | 3551 | CG | ASN G | 532 | −20.304 | 0.165 | −92.642 | 1.00 | 67.75 | | C |
| ATOM | 3552 | OD1 | ASN G | 532 | −19.416 | 0.312 | −93.481 | 1.00 | 53.59 | | O |
| ATOM | 3553 | ND2 | ASN G | 532 | −21.285 | −0.716 | −92.786 | 1.00 | 61.81 | | N |
| ATOM | 3554 | C | ASN G | 532 | −21.809 | 2.759 | −92.472 | 1.00 | 72.17 | | C |
| ATOM | 3555 | O | ASN G | 532 | −21.823 | 2.885 | −93.696 | 1.00 | 85.75 | | O |
| ATOM | 3556 | N | GLU G | 533 | −22.907 | 2.812 | −91.726 | 1.00 | 79.68 | | N |
| ATOM | 3557 | CA | GLU G | 533 | −24.220 | 3.041 | −92.307 | 1.00 | 71.96 | | C |
| ATOM | 3558 | CB | GLU G | 533 | −25.300 | 2.936 | −91.234 | 1.00 | 67.87 | | C |
| ATOM | 3559 | CG | GLU G | 533 | −26.680 | 2.653 | −91.788 | 1.00 | 81.94 | | C |
| ATOM | 3560 | CD | GLU G | 533 | −26.710 | 1.397 | −92.632 | 1.00 | 92.79 | | C |
| ATOM | 3561 | OE1 | GLU G | 533 | −26.313 | 0.331 | −92.119 | 1.00 | 95.90 | | O |
| ATOM | 3562 | OE2 | GLU G | 533 | −26.989 | 1.501 | −93.843 | 1.00 | 93.14 | | O |
| ATOM | 3563 | C | GLU G | 533 | −24.296 | 4.406 | −92.974 | 1.00 | 68.54 | | C |
| ATOM | 3564 | O | GLU G | 533 | −25.101 | 4.616 | −93.879 | 1.00 | 79.29 | | O |
| ATOM | 3565 | N | ILE G | 534 | −23.542 | 5.359 | −92.440 | 1.00 | 69.50 | | N |
| ATOM | 3566 | CA | ILE G | 534 | −23.498 | 6.702 | −93.003 | 1.00 | 72.55 | | C |
| ATOM | 3567 | CB | ILE G | 534 | −23.070 | 7.737 | −91.956 | 1.00 | 79.55 | | C |
| ATOM | 3568 | CG1 | ILE G | 534 | −24.053 | 7.740 | −90.784 | 1.00 | 80.41 | | C |
| ATOM | 3569 | CD1 | ILE G | 534 | −25.321 | 6.951 | −91.049 | 1.00 | 89.38 | | C |
| ATOM | 3570 | CG2 | ILE G | 534 | −22.981 | 9.118 | −92.588 | 1.00 | 78.63 | | C |
| ATOM | 3571 | C | ILE G | 534 | −22.538 | 6.766 | −94.180 | 1.00 | 74.92 | | C |
| ATOM | 3572 | O | ILE G | 534 | −22.589 | 7.693 | −94.987 | 1.00 | 81.01 | | O |
| ATOM | 3573 | N | ALA G | 535 | −21.648 | 5.786 | −94.255 | 1.00 | 79.52 | | N |
| ATOM | 3574 | CA | ALA G | 535 | −20.721 | 5.683 | −95.368 | 1.00 | 82.64 | | C |
| ATOM | 3575 | CB | ALA G | 535 | −19.529 | 4.824 | −94.974 | 1.00 | 75.39 | | C |
| ATOM | 3576 | C | ALA G | 535 | −21.416 | 5.115 | −96.604 | 1.00 | 81.39 | | C |
| ATOM | 3577 | O | ALA G | 535 | −21.170 | 5.557 | −97.727 | 1.00 | 85.54 | | O |
| ATOM | 3578 | N | ARG G | 536 | −22.325 | 4.171 | −96.380 | 1.00 | 78.41 | | N |
| ATOM | 3579 | CA | ARG G | 536 | −23.115 | 3.581 | −97.457 | 1.00 | 83.27 | | C |
| ATOM | 3580 | CB | ARG G | 536 | −23.781 | 2.287 | −96.984 | 1.00 | 84.58 | | C |
| ATOM | 3581 | CG | ARG G | 536 | −22.808 | 1.180 | −96.618 | 1.00 | 103.24 | | C |
| ATOM | 3582 | CD | ARG G | 536 | −23.316 | −0.171 | −97.085 | 1.00 | 114.72 | | C |
| ATOM | 3583 | NE | ARG G | 536 | −24.587 | −0.515 | −96.457 | 1.00 | 117.80 | | N |
| ATOM | 3584 | CZ | ARG G | 536 | −25.297 | −1.600 | −96.750 | 1.00 | 116.66 | | C |
| ATOM | 3585 | NH1 | ARG G | 536 | −24.875 | −2.439 | −97.685 | 1.00 | 114.57 | | N |
| ATOM | 3586 | NH2 | ARG G | 536 | −26.417 | −1.862 | −96.089 | 1.00 | 116.38 | | N |
| ATOM | 3587 | C | ARG G | 536 | −24.172 | 4.552 | −97.973 | 1.00 | 82.20 | | C |
| ATOM | 3588 | O | ARG G | 536 | −24.315 | 4.737 | −99.182 | 1.00 | 86.28 | | O |
| ATOM | 3589 | N | ILE G | 537 | −24.941 | 5.135 | −97.058 | 1.00 | 77.08 | | N |
| ATOM | 3590 | CA | ILE G | 537 | −25.917 | 6.156 | −97.430 | 1.00 | 66.79 | | C |
| ATOM | 3591 | CB | ILE G | 537 | −26.719 | 6.680 | −96.216 | 1.00 | 64.76 | | C |
| ATOM | 3592 | CG1 | ILE G | 537 | −27.614 | 5.583 | −95.640 | 1.00 | 66.92 | | C |
| ATOM | 3593 | CD1 | ILE G | 537 | −28.487 | 6.055 | −94.493 | 1.00 | 70.83 | | C |
| ATOM | 3594 | CG2 | ILE G | 537 | −27.593 | 7.848 | −96.631 | 1.00 | 51.02 | | C |
| ATOM | 3595 | C | ILE G | 537 | −25.221 | 7.330 | −98.108 | 1.00 | 74.94 | | C |
| ATOM | 3596 | O | ILE G | 537 | −25.824 | 8.043 | −98.907 | 1.00 | 97.16 | | O |
| ATOM | 3597 | N | LYS G | 538 | −23.941 | 7.514 | −97.804 | 1.00 | 75.09 | | N |
| ATOM | 3598 | CA | LYS G | 538 | −23.162 | 8.575 | −98.427 | 1.00 | 90.28 | | C |
| ATOM | 3599 | CB | LYS G | 538 | −22.051 | 9.054 | −97.491 | 1.00 | 93.73 | | C |
| ATOM | 3600 | CG | LYS G | 538 | −21.584 | 10.479 | −97.760 | 1.00 | 96.60 | | C |
| ATOM | 3601 | CD | LYS G | 538 | −20.446 | 10.876 | −96.830 | 1.00 | 95.37 | | C |
| ATOM | 3602 | CE | LYS G | 538 | −20.008 | 12.313 | −97.067 | 1.00 | 100.63 | | C |
| ATOM | 3603 | NZ | LYS G | 538 | −18.989 | 12.750 | −96.074 | 1.00 | 99.58 | | N |
| ATOM | 3604 | C | LYS G | 538 | −22.574 | 8.114 | −99.754 | 1.00 | 102.20 | | C |
| ATOM | 3605 | O | LYS G | 538 | −21.940 | 8.891 | −100.466 | 1.00 | 112.11 | | O |
| ATOM | 3606 | N | LYS G | 539 | −22.870 | 6.874 | −100.126 | 1.00 | 105.15 | | N |
| ATOM | 3607 | CA | LYS G | 539 | −22.411 | 6.328 | −101.396 | 1.00 | 105.65 | | C |
| ATOM | 3608 | CB | LYS G | 539 | −21.904 | 4.897 | −101.211 | 1.00 | 110.12 | | C |
| ATOM | 3609 | CG | LYS G | 539 | −21.426 | 4.246 | −102.498 | 1.00 | 113.31 | | C |
| ATOM | 3610 | CD | LYS G | 539 | −21.023 | 2.794 | −102.284 | 1.00 | 114.26 | | C |
| ATOM | 3611 | CE | LYS G | 539 | −20.414 | 2.201 | −103.550 | 1.00 | 118.39 | | C |
| ATOM | 3612 | NZ | LYS G | 539 | −19.511 | 1.047 | −103.263 | 1.00 | 121.77 | | N |
| ATOM | 3613 | C | LYS G | 539 | −23.517 | 6.364 | −102.444 | 1.00 | 108.88 | | C |
| ATOM | 3614 | O | LYS G | 539 | −23.375 | 5.808 | −103.532 | 1.00 | 110.77 | | O |
| ATOM | 3615 | N | LEU G | 540 | −24.625 | 7.011 | −102.102 | 1.00 | 111.50 | | N |
| ATOM | 3616 | CA | LEU G | 540 | −25.722 | 7.201 | −103.038 | 1.00 | 110.31 | | C |
| ATOM | 3617 | CB | LEU G | 540 | −27.025 | 6.656 | −102.451 | 1.00 | 101.19 | | C |
| ATOM | 3618 | CG | LEU G | 540 | −26.983 | 5.200 | −101.978 | 1.00 | 94.26 | | C |
| ATOM | 3619 | CD1 | LEU G | 540 | −28.376 | 4.691 | −101.683 | 1.00 | 86.40 | | C |
| ATOM | 3620 | CD2 | LEU G | 540 | −26.307 | 4.311 | −103.004 | 1.00 | 84.54 | | C |
| ATOM | 3621 | C | LEU G | 540 | −25.865 | 8.679 | −103.374 | 1.00 | 118.37 | | C |
| ATOM | 3622 | O | LEU G | 540 | −26.802 | 9.081 | −104.064 | 1.00 | 116.71 | | O |
| ATOM | 3623 | N | ILE G | 541 | −24.894 | 9.474 | −102.934 | 1.00 | 130.80 | | N |
| ATOM | 3624 | CA | ILE G | 541 | −24.844 | 10.896 | −103.267 | 1.00 | 140.48 | | C |
| ATOM | 3625 | CB | ILE G | 541 | −25.701 | 11.746 | −102.294 | 1.00 | 138.99 | | C |
| ATOM | 3626 | CG1 | ILE G | 541 | −27.145 | 11.234 | −102.257 | 1.00 | 133.30 | | C |
| ATOM | 3627 | CD1 | ILE G | 541 | −28.050 | 11.879 | −103.291 | 1.00 | 136.26 | | C |
| ATOM | 3628 | CG2 | ILE G | 541 | −25.680 | 13.218 | −102.707 | 1.00 | 136.12 | | C |
| ATOM | 3629 | C | ILE G | 541 | −23.407 | 11.410 | −103.250 | 1.00 | 144.97 | | C |
| ATOM | 3630 | O | ILE G | 541 | −23.116 | 12.480 | −103.783 | 1.00 | 145.83 | | O |
| ATOM | 3631 | N | GLY G | 542 | −22.519 | 10.663 | −102.602 | 1.00 | 148.77 | | N |
| ATOM | 3632 | CA | GLY G | 542 | −21.119 | 11.060 | −102.505 | 1.00 | 153.58 | | C |
| ATOM | 3633 | C | GLY G | 542 | −20.209 | 10.194 | −103.357 | 1.00 | 160.45 | | C |
| ATOM | 3634 | O | GLY G | 542 | −20.636 | 9.643 | −104.372 | 1.00 | 163.41 | | O |
| ATOM | 3635 | N | GLU G | 543 | −18.962 | 10.042 | −102.921 | 1.00 | 163.83 | | N |
| ATOM | 3636 | CA | GLU G | 543 | −17.983 | 9.236 | −103.649 | 1.00 | 165.52 | | C |
| ATOM | 3637 | CB | GLU G | 543 | −16.557 | 9.674 | −103.305 | 1.00 | 158.94 | | C |
| ATOM | 3642 | C | GLU G | 543 | −18.156 | 7.742 | −103.381 | 1.00 | 168.27 | | C |
| ATOM | 3643 | O | GLU G | 543 | −19.116 | 7.322 | −102.736 | 1.00 | 167.37 | | O |
| ATOM | 3644 | N | GLN H | 26 | −24.590 | 4.282 | 7.491 | 1.00 | 23.62 | B | N |
| ATOM | 3645 | CA | GLN H | 26 | −23.869 | 3.499 | 6.449 | 1.00 | 23.82 | B | C |
| ATOM | 3646 | CB | GLN H | 26 | −22.633 | 2.827 | 7.055 | 1.00 | 23.94 | B | C |
| ATOM | 3647 | CG | GLN H | 26 | −21.724 | 3.775 | 7.833 | 1.00 | 28.83 | B | C |
| ATOM | 3648 | CD | GLN H | 26 | −22.314 | 4.205 | 9.165 | 1.00 | 36.36 | B | C |
| ATOM | 3649 | OE1 | GLN H | 26 | −23.057 | 5.183 | 9.238 | 1.00 | 39.09 | B | O |
| ATOM | 3650 | NE2 | GLN H | 26 | −21.891 | 3.545 | 10.238 | 1.00 | 37.86 | B | N |
| ATOM | 3651 | C | GLN H | 26 | −24.797 | 2.460 | 5.813 | 1.00 | 23.09 | B | C |
| ATOM | 3652 | O | GLN H | 26 | −25.818 | 2.096 | 6.397 | 1.00 | 24.21 | B | O |
| ATOM | 3653 | N | ASN H | 27 | −24.543 | 2.146 | 4.544 | 1.00 | 20.98 | B | N |
| ATOM | 3654 | CA | ASN H | 27 | −25.343 | 1.173 | 3.786 | 1.00 | 19.70 | B | C |
| ATOM | 3655 | CB | ASN H | 27 | −26.642 | 1.832 | 3.269 | 1.00 | 20.41 | B | C |
| ATOM | 3656 | CG | ASN H | 27 | −27.524 | 0.887 | 2.423 | 1.00 | 23.56 | B | C |
| ATOM | 3657 | OD1 | ASN H | 27 | −27.835 | −0.233 | 2.837 | 1.00 | 24.42 | B | O |
| ATOM | 3658 | ND2 | ASN H | 27 | −27.836 | 1.322 | 1.182 | 1.00 | 27.70 | B | N |
| ATOM | 3659 | C | ASN H | 27 | −24.507 | 0.652 | 2.619 | 1.00 | 18.42 | B | C |
| ATOM | 3660 | O | ASN H | 27 | −24.904 | 0.783 | 1.462 | 1.00 | 18.47 | B | O |
| ATOM | 3661 | N | ILE H | 28 | −23.291 | 0.196 | 2.907 | 1.00 | 16.54 | B | N |
| ATOM | 3662 | CA | ILE H | 28 | −22.439 | −0.335 | 1.849 | 1.00 | 14.59 | B | C |
| ATOM | 3663 | CB | ILE H | 28 | −20.939 | −0.378 | 2.223 | 1.00 | 14.01 | B | C |
| ATOM | 3664 | CG1 | ILE H | 28 | −20.746 | −0.823 | 3.674 | 1.00 | 15.60 | B | C |
| ATOM | 3665 | CD1 | ILE H | 28 | −20.221 | −2.240 | 3.815 | 1.00 | 16.26 | B | C |
| ATOM | 3666 | CG2 | ILE H | 28 | −20.276 | 0.964 | 1.942 | 1.00 | 12.57 | B | C |
| ATOM | 3667 | C | ILE H | 28 | −22.898 | −1.700 | 1.360 | 1.00 | 13.86 | B | C |
| ATOM | 3668 | O | ILE H | 28 | −23.458 | −2.494 | 2.119 | 1.00 | 13.70 | B | O |
| ATOM | 3669 | N | THR H | 29 | −22.868 | −1.850 | 0.044 | 1.00 | 13.23 | B | N |
| ATOM | 3670 | CA | THR H | 29 | −23.353 | −3.045 | −0.612 | 1.00 | 12.65 | B | C |
| ATOM | 3671 | CB | THR H | 29 | −24.751 | −2.816 | −1.206 | 1.00 | 12.69 | B | C |
| ATOM | 3672 | OG1 | THR H | 29 | −25.331 | −1.643 | −0.623 | 1.00 | 12.62 | B | O |
| ATOM | 3673 | CG2 | THR H | 29 | −25.650 | −4.012 | −0.934 | 1.00 | 12.92 | B | C |
| ATOM | 3674 | C | THR H | 29 | −22.392 | −3.355 | −1.741 | 1.00 | 12.47 | B | C |
| ATOM | 3675 | O | THR H | 29 | −21.760 | −2.457 | −2.297 | 1.00 | 12.53 | B | O |
| ATOM | 3676 | N | GLU H | 30 | −22.300 | −4.626 | −2.095 | 1.00 | 11.98 | B | N |
| ATOM | 3677 | CA | GLU H | 30 | −21.668 | −5.007 | −3.338 | 1.00 | 11.68 | B | C |
| ATOM | 3678 | CB | GLU H | 30 | −20.393 | −5.803 | −3.049 | 1.00 | 11.98 | B | C |
| ATOM | 3679 | CG | GLU H | 30 | −19.274 | −5.593 | −4.058 | 1.00 | 13.38 | B | C |
| ATOM | 3680 | CD | GLU H | 30 | −17.927 | −6.054 | −3.533 | 1.00 | 16.03 | B | C |
| ATOM | 3681 | OE1 | GLU H | 30 | −17.846 | −7.190 | −3.019 | 1.00 | 16.65 | B | O |
| ATOM | 3682 | OE2 | GLU H | 30 | −16.968 | −5.253 | −3.563 | 1.00 | 17.06 | B | O |
| ATOM | 3683 | C | GLU H | 30 | −22.655 | −5.860 | −4.114 | 1.00 | 11.39 | B | C |
| ATOM | 3684 | O | GLU H | 30 | −23.316 | −6.727 | −3.542 | 1.00 | 11.50 | B | O |
| ATOM | 3685 | N | GLU H | 31 | −22.850 | −5.534 | −5.384 | 1.00 | 11.11 | B | N |
| ATOM | 3686 | CA | GLU H | 31 | −23.602 | −6.419 | −6.248 | 1.00 | 11.06 | B | C |
| ATOM | 3687 | CB | GLU H | 31 | −24.955 | −5.813 | −6.611 | 1.00 | 11.48 | B | C |
| ATOM | 3688 | CG | GLU H | 31 | −25.093 | −5.417 | −8.063 | 1.00 | 13.90 | B | C |
| ATOM | 3689 | CD | GLU H | 31 | −26.500 | −4.981 | −8.404 | 1.00 | 17.27 | B | C |
| ATOM | 3690 | OE1 | GLU H | 31 | −27.092 | −5.567 | −9.335 | 1.00 | 17.55 | B | O |
| ATOM | 3691 | OE2 | GLU H | 31 | −27.053 | −4.130 | −7.676 | 1.00 | 18.73 | B | O |
| ATOM | 3692 | C | GLU H | 31 | −22.828 | −6.839 | −7.485 | 1.00 | 10.47 | B | C |
| ATOM | 3693 | O | GLU H | 31 | −22.042 | −6.071 | −8.040 | 1.00 | 10.12 | B | O |
| ATOM | 3694 | N | PHE H | 32 | −22.962 | −8.116 | −7.818 | 1.00 | 10.15 | B | N |
| ATOM | 3695 | CA | PHE H | 32 | −22.042 | −8.788 | −8.716 | 1.00 | 9.62 | B | C |
| ATOM | 3696 | CB | PHE H | 32 | −21.692 | −10.167 | −8.158 | 1.00 | 9.42 | B | C |
| ATOM | 3697 | CG | PHE H | 32 | −21.130 | −11.106 | −9.179 | 1.00 | 9.27 | B | C |
| ATOM | 3698 | CD1 | PHE H | 32 | −19.865 | −10.896 | −9.702 | 1.00 | 9.37 | B | C |
| ATOM | 3699 | CE1 | PHE H | 32 | −19.315 | −11.784 | −10.605 | 1.00 | 9.52 | B | C |
| ATOM | 3700 | CZ | PHE H | 32 | −20.027 | −12.905 | −10.989 | 1.00 | 9.82 | B | C |
| ATOM | 3701 | CE2 | PHE H | 32 | −21.294 | −13.127 | −10.472 | 1.00 | 10.13 | B | C |
| ATOM | 3702 | CD2 | PHE H | 32 | −21.822 | −12.250 | −9.544 | 1.00 | 10.06 | B | C |
| ATOM | 3703 | C | PHE H | 32 | −22.707 | −8.941 | −10.074 | 1.00 | 9.64 | B | C |
| ATOM | 3704 | O | PHE H | 32 | −23.887 | −9.282 | −10.159 | 1.00 | 9.84 | B | O |
| ATOM | 3705 | N | TYR H | 33 | −21.965 | −8.638 | −11.132 | 1.00 | 9.59 | B | N |
| ATOM | 3706 | CA | TYR H | 33 | −22.526 | −8.672 | −12.474 | 1.00 | 9.67 | B | C |
| ATOM | 3707 | CB | TYR H | 33 | −22.259 | −7.360 | −13.207 | 1.00 | 9.52 | B | C |
| ATOM | 3708 | CG | TYR H | 33 | −23.038 | −6.196 | −12.645 | 1.00 | 10.94 | B | C |
| ATOM | 3709 | CD1 | TYR H | 33 | −22.482 | −5.372 | −11.677 | 1.00 | 12.23 | B | C |
| ATOM | 3710 | CE1 | TYR H | 33 | −23.198 | −4.323 | −11.137 | 1.00 | 12.93 | B | C |
| ATOM | 3711 | CZ | TYR H | 33 | −24.510 | −4.129 | −11.510 | 1.00 | 13.47 | B | C |
| ATOM | 3712 | OH | TYR H | 33 | −25.231 | −3.093 | −10.963 | 1.00 | 14.54 | B | O |
| ATOM | 3713 | CE2 | TYR H | 33 | −25.100 | −4.963 | −12.436 | 1.00 | 13.56 | B | C |
| ATOM | 3714 | CD2 | TYR H | 33 | −24.373 | −6.006 | −12.976 | 1.00 | 12.96 | B | C |
| ATOM | 3715 | C | TYR H | 33 | −22.001 | −9.853 | −13.273 | 1.00 | 9.94 | B | C |
| ATOM | 3716 | O | TYR H | 33 | −20.998 | −9.747 | −13.978 | 1.00 | 10.16 | B | O |
| ATOM | 3717 | N | GLN H | 34 | −22.714 | −10.969 | −13.184 | 1.00 | 10.06 | B | N |
| ATOM | 3718 | CA | GLN H | 34 | −22.191 | −12.249 | −13.632 | 1.00 | 10.01 | B | C |
| ATOM | 3719 | CB | GLN H | 34 | −23.190 | −13.361 | −13.327 | 1.00 | 10.22 | B | C |
| ATOM | 3720 | CG | GLN H | 34 | −22.760 | −14.720 | −13.829 | 1.00 | 11.12 | B | C |
| ATOM | 3721 | CD | GLN H | 34 | −23.604 | −15.836 | −13.257 | 1.00 | 13.13 | B | C |
| ATOM | 3722 | OE1 | GLN H | 34 | −24.746 | −16.042 | −13.673 | 1.00 | 14.24 | B | O |
| ATOM | 3723 | NE2 | GLN H | 34 | −23.057 | −16.550 | −12.278 | 1.00 | 13.79 | B | N |
| ATOM | 3724 | C | GLN H | 34 | −21.889 | −12.220 | −15.123 | 1.00 | 9.94 | B | C |
| ATOM | 3725 | O | GLN H | 34 | −21.096 | −13.018 | −15.622 | 1.00 | 10.08 | B | O |
| ATOM | 3726 | N | SER H | 35 | −22.564 | −11.324 | −15.836 | 1.00 | 9.87 | B | N |
| ATOM | 3727 | CA | SER H | 35 | −22.458 | −11.253 | −17.290 | 1.00 | 9.91 | B | C |
| ATOM | 3728 | CB | SER H | 35 | −23.743 | −10.676 | −17.889 | 1.00 | 10.03 | B | C |
| ATOM | 3729 | OG | SER H | 35 | −24.034 | −9.401 | −17.343 | 1.00 | 10.81 | B | O |
| ATOM | 3730 | C | SER H | 35 | −21.256 | −10.410 | −17.713 | 1.00 | 9.56 | B | C |
| ATOM | 3731 | O | SER H | 35 | −21.002 | −10.214 | −18.903 | 1.00 | 9.59 | B | O |
| ATOM | 3732 | N | THR H | 36 | −20.539 | −9.892 | −16.724 | 1.00 | 9.21 | B | N |
| ATOM | 3733 | CA | THR H | 36 | −19.420 | −8.996 | −16.964 | 1.00 | 9.06 | B | C |
| ATOM | 3734 | CB | THR H | 36 | −19.817 | −7.536 | −16.686 | 1.00 | 9.49 | B | C |
| ATOM | 3735 | OG1 | THR H | 36 | −20.874 | −7.149 | −17.573 | 1.00 | 10.82 | B | O |
| ATOM | 3736 | CG2 | THR H | 36 | −18.629 | −6.609 | −16.883 | 1.00 | 9.45 | B | C |
| ATOM | 3737 | C | THR H | 36 | −18.292 | −9.403 | −16.029 | 1.00 | 8.77 | B | C |
| ATOM | 3738 | O | THR H | 36 | −17.298 | −8.693 | −15.882 | 1.00 | 8.60 | B | O |
| ATOM | 3739 | N | CYS H | 37 | −18.457 | −10.572 | −15.415 | 1.00 | 8.69 | B | N |
| ATOM | 3740 | CA | CYS H | 37 | −17.594 | −11.011 | −14.327 | 1.00 | 8.69 | B | C |
| ATOM | 3741 | CB | CYS H | 37 | −16.509 | −11.955 | −14.846 | 1.00 | 8.81 | B | C |
| ATOM | 3742 | SG | CYS H | 37 | −15.737 | −12.977 | −13.569 | 1.00 | 10.80 | B | S |
| ATOM | 3743 | C | CYS H | 37 | −16.958 | −9.820 | −13.631 | 1.00 | 8.27 | B | C |
| ATOM | 3744 | O | CYS H | 37 | −15.735 | −9.671 | −13.628 | 1.00 | 8.27 | B | O |
| ATOM | 3745 | N | SER H | 38 | −17.796 | −8.950 | −13.080 | 1.00 | 7.92 | B | N |
| ATOM | 3746 | CA | SER H | 38 | −17.309 | −7.784 | −12.360 | 1.00 | 7.51 | B | C |
| ATOM | 3747 | CB | SER H | 38 | −17.130 | −6.595 | −13.308 | 1.00 | 7.63 | B | C |
| ATOM | 3748 | OG | SER H | 38 | −18.369 | −6.193 | −13.864 | 1.00 | 8.54 | B | O |
| ATOM | 3749 | C | SER H | 38 | −18.227 | −7.411 | −11.207 | 1.00 | 7.02 | B | C |
| ATOM | 3750 | O | SER H | 38 | −19.404 | −7.771 | −11.192 | 1.00 | 6.83 | B | O |
| ATOM | 3751 | N | ALA H | 39 | −17.647 | −6.784 | −10.191 | 1.00 | 6.64 | B | N |
| ATOM | 3752 | CA | ALA H | 39 | −18.371 | −6.478 | −8.969 | 1.00 | 6.58 | B | C |
| ATOM | 3753 | CB | ALA H | 39 | −17.843 | −7.320 | −7.817 | 1.00 | 6.67 | B | C |
| ATOM | 3754 | C | ALA H | 39 | −18.242 | −4.995 | −8.651 | 1.00 | 6.93 | B | C |
| ATOM | 3755 | O | ALA H | 39 | −17.217 | −4.378 | −8.944 | 1.00 | 7.38 | B | O |
| ATOM | 3756 | N | VAL H | 40 | −19.333 | −4.401 | −8.179 | 1.00 | 6.87 | B | N |
| ATOM | 3757 | CA | VAL H | 40 | −19.316 | −3.008 | −7.746 | 1.00 | 6.55 | B | C |
| ATOM | 3758 | CB | VAL H | 40 | −20.208 | −2.122 | −8.635 | 1.00 | 6.22 | B | C |
| ATOM | 3759 | CG1 | VAL H | 40 | −20.465 | −0.783 | −7.959 | 1.00 | 6.52 | B | C |
| ATOM | 3760 | CG2 | VAL H | 40 | −19.564 | −1.921 | −9.998 | 1.00 | 6.62 | B | C |
| ATOM | 3761 | C | VAL H | 40 | −19.743 | −2.862 | −6.290 | 1.00 | 6.73 | B | C |
| ATOM | 3762 | O | VAL H | 40 | −20.805 | −3.340 | −5.891 | 1.00 | 6.83 | B | O |
| ATOM | 3763 | N | SER H | 41 | −18.883 | −2.240 | −5.492 | 1.00 | 6.86 | B | N |
| ATOM | 3764 | CA | SER H | 41 | −19.251 | −1.825 | −4.148 | 1.00 | 6.75 | B | C |
| ATOM | 3765 | CB | SER H | 41 | −18.020 | −1.779 | −3.242 | 1.00 | 7.06 | B | C |
| ATOM | 3766 | OG | SER H | 41 | −16.905 | −2.388 | −3.866 | 1.00 | 7.50 | B | O |
| ATOM | 3767 | C | SER H | 41 | −19.922 | −0.461 | −4.180 | 1.00 | 6.56 | B | C |
| ATOM | 3768 | O | SER H | 41 | −19.392 | 0.490 | −4.756 | 1.00 | 6.08 | B | O |
| ATOM | 3769 | N | LYS H | 42 | −21.097 | −0.378 | −3.565 | 1.00 | 7.02 | B | N |
| ATOM | 3770 | CA | LYS H | 42 | −21.938 | 0.810 | −3.662 | 1.00 | 7.83 | B | C |
| ATOM | 3771 | CB | LYS H | 42 | −23.296 | 0.459 | −4.269 | 1.00 | 8.15 | B | C |
| ATOM | 3772 | CG | LYS H | 42 | −23.226 | −0.171 | −5.641 | 1.00 | 9.75 | B | C |
| ATOM | 3773 | CD | LYS H | 42 | −24.621 | −0.473 | −6.153 | 1.00 | 12.62 | B | C |
| ATOM | 3774 | CE | LYS H | 42 | −24.579 | −1.069 | −7.548 | 1.00 | 14.04 | B | C |
| ATOM | 3775 | NZ | LYS H | 42 | −25.858 | −0.856 | −8.279 | 1.00 | 14.96 | B | N |
| ATOM | 3776 | C | LYS H | 42 | −22.143 | 1.444 | −2.290 | 1.00 | 7.80 | B | C |
| ATOM | 3777 | O | LYS H | 42 | −21.947 | 0.798 | −1.258 | 1.00 | 8.15 | B | O |
| ATOM | 3778 | N | GLY H | 43 | −22.594 | 2.696 | −2.290 | 1.00 | 7.59 | B | N |
| ATOM | 3779 | CA | GLY H | 43 | −23.140 | 3.321 | −1.090 | 1.00 | 7.35 | B | C |
| ATOM | 3780 | C | GLY H | 43 | −22.131 | 4.200 | −0.378 | 1.00 | 7.10 | B | C |
| ATOM | 3781 | O | GLY H | 43 | −22.239 | 4.439 | 0.824 | 1.00 | 7.13 | B | O |
| ATOM | 3782 | N | TYR H | 44 | −21.173 | 4.723 | −1.136 | 1.00 | 7.01 | B | N |
| ATOM | 3783 | CA | TYR H | 44 | −20.106 | 5.549 | −0.577 | 1.00 | 6.89 | B | C |
| ATOM | 3784 | CB | TYR H | 44 | −18.755 | 5.125 | −1.160 | 1.00 | 6.88 | B | C |
| ATOM | 3785 | CG | TYR H | 44 | −18.296 | 3.748 | −0.732 | 1.00 | 6.41 | B | C |
| ATOM | 3786 | CD1 | TYR H | 44 | −18.340 | 2.676 | −1.615 | 1.00 | 6.62 | B | C |
| ATOM | 3787 | CE1 | TYR H | 44 | −17.871 | 1.427 | −1.245 | 1.00 | 6.48 | B | C |
| ATOM | 3788 | CZ | TYR H | 44 | −17.312 | 1.253 | 0.005 | 1.00 | 6.88 | B | C |
| ATOM | 3789 | OH | TYR H | 44 | −16.834 | 0.020 | 0.386 | 1.00 | 7.50 | B | O |
| ATOM | 3790 | CE2 | TYR H | 44 | −17.197 | 2.318 | 0.870 | 1.00 | 6.68 | B | C |
| ATOM | 3791 | CD2 | TYR H | 44 | −17.670 | 3.559 | 0.492 | 1.00 | 5.91 | B | C |
| ATOM | 3792 | C | TYR H | 44 | −20.357 | 7.026 | −0.886 | 1.00 | 6.86 | B | C |
| ATOM | 3793 | O | TYR H | 44 | −20.912 | 7.354 | −1.933 | 1.00 | 7.14 | B | O |
| ATOM | 3794 | N | LEU H | 45 | −19.834 | 7.916 | −0.045 | 1.00 | 6.44 | B | N |
| ATOM | 3795 | CA | LEU H | 45 | −20.073 | 9.356 | −0.205 | 1.00 | 6.12 | B | C |
| ATOM | 3796 | CB | LEU H | 45 | −20.896 | 9.917 | 0.966 | 1.00 | 6.31 | B | C |
| ATOM | 3797 | CG | LEU H | 45 | −22.355 | 9.463 | 1.108 | 1.00 | 6.38 | B | C |
| ATOM | 3798 | CD1 | LEU H | 45 | −22.859 | 9.699 | 2.524 | 1.00 | 7.05 | B | C |
| ATOM | 3799 | CD2 | LEU H | 45 | −23.256 | 10.158 | 0.096 | 1.00 | 7.05 | B | C |
| ATOM | 3800 | C | LEU H | 45 | −18.791 | 10.173 | −0.413 | 1.00 | 5.84 | B | C |
| ATOM | 3801 | O | LEU H | 45 | −17.966 | 10.289 | 0.493 | 1.00 | 5.94 | B | O |
| ATOM | 3802 | N | SER H | 46 | −18.764 | 10.924 | −1.512 | 1.00 | 5.43 | B | N |
| ATOM | 3803 | CA | SER H | 46 | −17.528 | 11.442 | −2.112 | 1.00 | 5.26 | B | C |
| ATOM | 3804 | CB | SER H | 46 | −17.801 | 11.821 | −3.568 | 1.00 | 5.21 | B | C |
| ATOM | 3805 | OG | SER H | 46 | −18.827 | 12.800 | −3.647 | 1.00 | 4.95 | B | O |
| ATOM | 3806 | C | SER H | 46 | −17.113 | 12.696 | −1.355 | 1.00 | 5.51 | B | C |
| ATOM | 3807 | O | SER H | 46 | −17.968 | 13.359 | −0.771 | 1.00 | 6.03 | B | O |
| ATOM | 3808 | N | ALA H | 47 | −15.850 | 13.103 | −1.424 | 1.00 | 5.24 | B | N |
| ATOM | 3809 | CA | ALA H | 47 | −15.530 | 14.323 | −0.704 | 1.00 | 4.83 | B | C |
| ATOM | 3810 | CB | ALA H | 47 | −16.269 | 14.344 | 0.622 | 1.00 | 5.01 | B | C |
| ATOM | 3811 | C | ALA H | 47 | −14.089 | 14.763 | −0.512 | 1.00 | 4.35 | B | C |
| ATOM | 3812 | O | ALA H | 47 | −13.178 | 13.936 | −0.449 | 1.00 | 4.45 | B | O |
| ATOM | 3813 | N | LEU H | 48 | −13.989 | 15.994 | −0.016 | 1.00 | 3.67 | B | N |
| ATOM | 3814 | CA | LEU H | 48 | −12.719 | 16.625 | 0.308 | 1.00 | 3.15 | B | C |
| ATOM | 3815 | CB | LEU H | 48 | −12.390 | 17.712 | −0.720 | 1.00 | 2.78 | B | C |
| ATOM | 3816 | CG | LEU H | 48 | −12.856 | 17.464 | −2.159 | 1.00 | 2.18 | B | C |
| ATOM | 3817 | CD1 | LEU H | 48 | −12.396 | 18.583 | −3.082 | 1.00 | 2.00 | B | C |
| ATOM | 3818 | CD2 | LEU H | 48 | −12.378 | 16.109 | −2.666 | 1.00 | 3.94 | B | C |
| ATOM | 3819 | C | LEU H | 48 | −12.768 | 17.232 | 1.707 | 1.00 | 3.06 | B | C |
| ATOM | 3820 | O | LEU H | 48 | −13.519 | 18.174 | 1.961 | 1.00 | 3.27 | B | O |
| ATOM | 3821 | N | ARG H | 49 | −11.879 | 16.758 | 2.571 | 1.00 | 2.91 | B | N |
| ATOM | 3822 | CA | ARG H | 49 | −11.733 | 17.305 | 3.912 | 1.00 | 2.88 | B | C |
| ATOM | 3823 | CB | ARG H | 49 | −10.649 | 16.538 | 4.668 | 1.00 | 2.69 | B | C |
| ATOM | 3824 | CG | ARG H | 49 | −11.032 | 16.140 | 6.075 | 1.00 | 2.15 | B | C |
| ATOM | 3825 | CD | ARG H | 49 | −9.882 | 16.395 | 7.026 | 1.00 | 2.00 | B | C |
| ATOM | 3826 | NE | ARG H | 49 | −9.728 | 17.816 | 7.320 | 1.00 | 5.18 | B | N |
| ATOM | 3827 | CZ | ARG H | 49 | −8.580 | 18.478 | 7.231 | 1.00 | 7.68 | B | C |
| ATOM | 3828 | NH1 | ARG H | 49 | −7.481 | 17.849 | 6.838 | 1.00 | 8.32 | B | N |
| ATOM | 3829 | NH2 | ARG H | 49 | −8.521 | 19.756 | 7.575 | 1.00 | 8.12 | B | N |
| ATOM | 3830 | C | ARG H | 49 | −11.364 | 18.780 | 3.843 | 1.00 | 3.09 | B | C |
| ATOM | 3831 | O | ARG H | 49 | −10.497 | 19.175 | 3.064 | 1.00 | 3.08 | B | O |
| ATOM | 3832 | N | THR H | 50 | −12.022 | 19.593 | 4.661 | 1.00 | 3.37 | B | N |
| ATOM | 3833 | CA | THR H | 50 | −11.594 | 20.970 | 4.855 | 1.00 | 3.55 | B | C |
| ATOM | 3834 | CB | THR H | 50 | −12.413 | 21.946 | 3.998 | 1.00 | 3.25 | B | C |
| ATOM | 3835 | OG1 | THR H | 50 | −13.807 | 21.633 | 4.109 | 1.00 | 2.66 | B | O |
| ATOM | 3836 | CG2 | THR H | 50 | −11.992 | 21.844 | 2.543 | 1.00 | 3.46 | B | C |
| ATOM | 3837 | C | THR H | 50 | −11.665 | 21.395 | 6.313 | 1.00 | 4.07 | B | C |
| ATOM | 3838 | O | THR H | 50 | −11.171 | 22.462 | 6.678 | 1.00 | 4.35 | B | O |
| ATOM | 3840 | CA | GLY H | 51 | −13.255 | 22.675 | 6.821 | 1.00 | 4.78 | B | C |
| ATOM | 3841 | C | GLY H | 51 | −11.930 | 22.228 | 7.407 | 1.00 | 4.98 | B | C |
| ATOM | 3842 | O | GLY H | 51 | −11.589 | 21.046 | 7.365 | 1.00 | 5.00 | B | O |
| ATOM | 3843 | N | TRP H | 52 | −11.162 | 23.180 | 7.925 | 1.00 | 5.34 | B | N |
| ATOM | 3844 | CA | TRP H | 52 | −9.845 | 22.878 | 8.465 | 1.00 | 5.42 | B | C |
| ATOM | 3845 | CB | TRP H | 52 | −8.754 | 23.540 | 7.632 | 1.00 | 5.75 | B | C |
| ATOM | 3846 | CG | TRP H | 52 | −8.691 | 23.029 | 6.243 | 1.00 | 6.38 | B | C |
| ATOM | 3847 | CD1 | TRP H | 52 | −9.100 | 23.678 | 5.117 | 1.00 | 7.27 | B | C |
| ATOM | 3848 | NE1 | TRP H | 52 | −8.926 | 22.875 | 4.019 | 1.00 | 7.46 | B | N |
| ATOM | 3849 | CE2 | TRP H | 52 | −8.445 | 21.660 | 4.430 | 1.00 | 7.15 | B | C |
| ATOM | 3850 | CD2 | TRP H | 52 | −8.315 | 21.710 | 5.832 | 1.00 | 7.02 | B | C |
| ATOM | 3851 | CE3 | TRP H | 52 | −7.799 | 20.595 | 6.500 | 1.00 | 6.83 | B | C |
| ATOM | 3852 | CZ3 | TRP H | 52 | −7.466 | 19.473 | 5.761 | 1.00 | 6.73 | B | C |
| ATOM | 3853 | CH2 | TRP H | 52 | −7.634 | 19.445 | 4.372 | 1.00 | 7.13 | B | C |
| ATOM | 3854 | CZ2 | TRP H | 52 | −8.111 | 20.529 | 3.689 | 1.00 | 6.82 | B | C |
| ATOM | 3855 | C | TRP H | 52 | −9.723 | 23.329 | 9.905 | 1.00 | 5.34 | B | C |
| ATOM | 3856 | O | TRP H | 52 | −10.279 | 24.359 | 10.289 | 1.00 | 5.47 | B | O |
| ATOM | 3857 | N | TYR H | 53 | −8.800 | 22.695 | 10.615 | 1.00 | 5.29 | B | N |
| ATOM | 3858 | CA | TYR H | 53 | −8.531 | 23.038 | 11.997 | 1.00 | 5.58 | B | C |
| ATOM | 3859 | CB | TYR H | 53 | −9.040 | 21.934 | 12.919 | 1.00 | 5.51 | B | C |
| ATOM | 3860 | CG | TYR H | 53 | −8.759 | 22.187 | 14.377 | 1.00 | 6.59 | B | C |
| ATOM | 3861 | CD1 | TYR H | 53 | −9.668 | 22.879 | 15.169 | 1.00 | 8.25 | B | C |
| ATOM | 3862 | CE1 | TYR H | 53 | −9.440 | 23.068 | 16.521 | 1.00 | 9.40 | B | C |
| ATOM | 3863 | CZ | TYR H | 53 | −8.293 | 22.555 | 17.096 | 1.00 | 9.24 | B | C |
| ATOM | 3864 | OH | TYR H | 53 | −8.053 | 22.750 | 18.439 | 1.00 | 9.61 | B | O |
| ATOM | 3865 | CE2 | TYR H | 53 | −7.360 | 21.893 | 16.321 | 1.00 | 8.78 | B | C |
| ATOM | 3866 | CD2 | TYR H | 53 | −7.600 | 21.709 | 14.971 | 1.00 | 7.93 | B | C |
| ATOM | 3867 | C | TYR H | 53 | −7.042 | 23.256 | 12.214 | 1.00 | 5.74 | B | C |
| ATOM | 3868 | O | TYR H | 53 | −6.259 | 22.306 | 12.223 | 1.00 | 6.19 | B | O |
| ATOM | 3869 | N | THR H | 54 | −6.645 | 24.521 | 12.296 | 1.00 | 6.04 | B | N |
| ATOM | 3870 | CA | THR H | 54 | −5.249 | 24.869 | 12.519 | 1.00 | 6.67 | B | C |
| ATOM | 3871 | CB | THR H | 54 | −5.032 | 26.391 | 12.438 | 1.00 | 6.97 | B | C |
| ATOM | 3872 | OG1 | THR H | 54 | −4.958 | 26.793 | 11.064 | 1.00 | 7.51 | B | O |
| ATOM | 3873 | CG2 | THR H | 54 | −3.747 | 26.788 | 13.147 | 1.00 | 7.98 | B | C |
| ATOM | 3874 | C | THR H | 54 | −4.775 | 24.360 | 13.877 | 1.00 | 6.97 | B | C |
| ATOM | 3875 | O | THR H | 54 | −5.576 | 24.173 | 14.792 | 1.00 | 7.32 | B | O |
| ATOM | 3876 | N | SER H | 55 | −3.479 | 24.086 | 13.987 | 1.00 | 7.24 | B | N |
| ATOM | 3877 | CA | SER H | 55 | −2.811 | 24.106 | 15.282 | 1.00 | 7.65 | B | C |
| ATOM | 3878 | CB | SER H | 55 | −3.110 | 22.830 | 16.072 | 1.00 | 7.69 | B | C |
| ATOM | 3879 | OG | SER H | 55 | −2.308 | 22.754 | 17.238 | 1.00 | 7.51 | B | O |
| ATOM | 3880 | C | SER H | 55 | −1.308 | 24.309 | 15.154 | 1.00 | 8.03 | B | C |
| ATOM | 3881 | O | SER H | 55 | −0.654 | 23.695 | 14.311 | 1.00 | 8.43 | B | O |
| ATOM | 3882 | N | VAL H | 56 | −0.760 | 25.106 | 16.063 | 1.00 | 8.14 | B | N |
| ATOM | 3883 | CA | VAL H | 56 | 0.600 | 25.601 | 15.930 | 1.00 | 8.41 | B | C |
| ATOM | 3884 | CB | VAL H | 56 | 0.672 | 27.116 | 16.193 | 1.00 | 8.72 | B | C |
| ATOM | 3885 | CG1 | VAL H | 56 | 1.868 | 27.723 | 15.477 | 1.00 | 9.29 | B | C |
| ATOM | 3886 | CG2 | VAL H | 56 | −0.619 | 27.792 | 15.756 | 1.00 | 9.51 | B | C |
| ATOM | 3887 | C | VAL H | 56 | 1.509 | 24.884 | 16.917 | 1.00 | 8.19 | B | C |
| ATOM | 3888 | O | VAL H | 56 | 1.348 | 25.011 | 18.129 | 1.00 | 8.54 | B | O |
| ATOM | 3889 | N | ILE H | 57 | 2.428 | 24.086 | 16.389 | 1.00 | 7.91 | B | N |
| ATOM | 3890 | CA | ILE H | 57 | 3.224 | 23.192 | 17.214 | 1.00 | 7.64 | B | C |
| ATOM | 3891 | CB | ILE H | 57 | 3.175 | 21.749 | 16.674 | 1.00 | 7.35 | B | C |
| ATOM | 3892 | CG1 | ILE H | 57 | 1.726 | 21.302 | 16.462 | 1.00 | 7.25 | B | C |
| ATOM | 3893 | CD1 | ILE H | 57 | 1.593 | 19.930 | 15.834 | 1.00 | 8.42 | B | C |
| ATOM | 3894 | CG2 | ILE H | 57 | 3.915 | 20.799 | 17.603 | 1.00 | 7.52 | B | C |
| ATOM | 3895 | C | ILE H | 57 | 4.670 | 23.670 | 17.258 | 1.00 | 7.69 | B | C |
| ATOM | 3896 | O | ILE H | 57 | 5.272 | 23.936 | 16.219 | 1.00 | 7.79 | B | O |
| ATOM | 3897 | N | THR H | 58 | 5.175 | 23.897 | 18.467 | 1.00 | 7.85 | B | N |
| ATOM | 3898 | CA | THR H | 58 | 6.465 | 24.554 | 18.659 | 1.00 | 8.15 | B | C |
| ATOM | 3899 | CB | THR H | 58 | 6.297 | 25.903 | 19.383 | 1.00 | 8.14 | B | C |
| ATOM | 3900 | OG1 | THR H | 58 | 4.946 | 26.360 | 19.239 | 1.00 | 8.62 | B | O |
| ATOM | 3901 | CG2 | THR H | 58 | 7.246 | 26.943 | 18.806 | 1.00 | 7.94 | B | C |
| ATOM | 3902 | C | THR H | 58 | 7.393 | 23.666 | 19.485 | 1.00 | 8.20 | B | C |
| ATOM | 3903 | O | THR H | 58 | 7.051 | 23.281 | 20.600 | 1.00 | 8.18 | B | O |
| ATOM | 3904 | N | ILE H | 59 | 8.565 | 23.351 | 18.941 | 1.00 | 8.54 | B | N |
| ATOM | 3905 | CA | ILE H | 59 | 9.482 | 22.397 | 19.572 | 1.00 | 9.23 | B | C |
| ATOM | 3906 | CB | ILE H | 59 | 9.604 | 21.112 | 18.729 | 1.00 | 8.92 | B | C |
| ATOM | 3907 | CG1 | ILE H | 59 | 8.231 | 20.482 | 18.496 | 1.00 | 9.27 | B | C |
| ATOM | 3908 | CD1 | ILE H | 59 | 8.253 | 19.315 | 17.533 | 1.00 | 10.89 | B | C |
| ATOM | 3909 | CG2 | ILE H | 59 | 10.559 | 20.130 | 19.387 | 1.00 | 8.74 | B | C |
| ATOM | 3910 | C | ILE H | 59 | 10.880 | 23.004 | 19.691 | 1.00 | 10.02 | B | C |
| ATOM | 3911 | O | ILE H | 59 | 11.474 | 23.380 | 18.684 | 1.00 | 10.20 | B | O |
| ATOM | 3912 | N | GLU H | 60 | 11.434 | 23.059 | 20.898 | 1.00 | 10.95 | B | N |
| ATOM | 3913 | CA | GLU H | 60 | 12.592 | 23.923 | 21.138 | 1.00 | 12.13 | B | C |
| ATOM | 3914 | CB | GLU H | 60 | 12.914 | 23.976 | 22.634 | 1.00 | 12.44 | B | C |
| ATOM | 3915 | CG | GLU H | 60 | 11.854 | 24.705 | 23.444 | 1.00 | 15.04 | B | C |
| ATOM | 3916 | CD | GLU H | 60 | 12.365 | 25.173 | 24.793 | 1.00 | 19.34 | B | C |
| ATOM | 3917 | OE1 | GLU H | 60 | 12.659 | 24.309 | 25.647 | 1.00 | 20.63 | B | O |
| ATOM | 3918 | OE2 | GLU H | 60 | 12.495 | 26.401 | 24.990 | 1.00 | 21.19 | B | O |
| ATOM | 3919 | C | GLU H | 60 | 13.816 | 23.509 | 20.301 | 1.00 | 12.46 | B | C |
| ATOM | 3920 | O | GLU H | 60 | 14.196 | 22.336 | 20.316 | 1.00 | 12.68 | B | O |
| ATOM | 3921 | N | LEU H | 61 | 14.390 | 24.428 | 19.510 | 1.00 | 12.80 | B | N |
| ATOM | 3922 | CA | LEU H | 61 | 15.786 | 24.207 | 19.132 | 1.00 | 12.71 | B | C |
| ATOM | 3923 | CB | LEU H | 61 | 16.470 | 25.295 | 18.316 | 1.00 | 12.55 | B | C |
| ATOM | 3924 | CG | LEU H | 61 | 17.932 | 24.803 | 18.355 | 1.00 | 12.39 | B | C |
| ATOM | 3925 | CD1 | LEU H | 61 | 18.053 | 23.370 | 17.848 | 1.00 | 13.14 | B | C |
| ATOM | 3926 | CD2 | LEU H | 61 | 18.944 | 25.717 | 17.683 | 1.00 | 12.50 | B | C |
| ATOM | 3927 | C | LEU H | 61 | 16.459 | 24.114 | 20.455 | 1.00 | 12.94 | B | C |
| ATOM | 3928 | O | LEU H | 61 | 16.827 | 25.135 | 21.048 | 1.00 | 12.78 | B | O |
| ATOM | 3929 | N | SER H | 62 | 16.080 | 23.014 | 21.081 | 1.00 | 13.61 | B | N |
| ATOM | 3930 | CA | SER H | 62 | 16.441 | 22.760 | 22.447 | 1.00 | 14.70 | B | C |
| ATOM | 3931 | CB | SER H | 62 | 15.805 | 21.454 | 22.924 | 1.00 | 14.52 | B | C |
| ATOM | 3932 | OG | SER H | 62 | 14.398 | 21.584 | 23.034 | 1.00 | 14.80 | B | O |
| ATOM | 3933 | C | SER H | 62 | 17.947 | 22.650 | 22.469 | 1.00 | 15.48 | B | C |
| ATOM | 3934 | O | SER H | 62 | 18.498 | 21.677 | 21.961 | 1.00 | 15.50 | B | O |
| ATOM | 3935 | N | ASN H | 63 | 18.597 | 23.777 | 22.721 | 1.00 | 16.61 | B | N |
| ATOM | 3936 | CA | ASN H | 63 | 20.030 | 23.839 | 22.523 | 1.00 | 17.77 | B | C |
| ATOM | 3937 | CB | ASN H | 63 | 20.519 | 25.279 | 22.439 | 1.00 | 17.73 | B | C |
| ATOM | 3938 | CG | ASN H | 63 | 21.686 | 25.433 | 21.493 | 1.00 | 18.52 | B | C |
| ATOM | 3939 | OD1 | ASN H | 63 | 22.559 | 24.567 | 21.428 | 1.00 | 18.29 | B | O |
| ATOM | 3940 | ND2 | ASN H | 63 | 21.641 | 26.467 | 20.665 | 1.00 | 19.40 | B | N |
| ATOM | 3941 | C | ASN H | 63 | 20.795 | 23.070 | 23.587 | 1.00 | 18.53 | B | C |
| ATOM | 3942 | O | ASN H | 63 | 20.356 | 22.967 | 24.732 | 1.00 | 18.82 | B | O |
| ATOM | 3943 | N | ILE H | 64 | 21.887 | 22.440 | 23.169 | 1.00 | 19.60 | B | N |
| ATOM | 3944 | CA | ILE H | 64 | 22.830 | 21.832 | 24.096 | 1.00 | 21.04 | B | C |
| ATOM | 3945 | CB | ILE H | 64 | 23.599 | 20.680 | 23.429 | 1.00 | 20.60 | B | C |
| ATOM | 3946 | CG1 | ILE H | 64 | 22.650 | 19.824 | 22.590 | 1.00 | 20.13 | B | C |
| ATOM | 3947 | CD1 | ILE H | 64 | 23.355 | 18.820 | 21.712 | 1.00 | 20.19 | B | C |
| ATOM | 3948 | CG2 | ILE H | 64 | 24.321 | 19.843 | 24.474 | 1.00 | 20.74 | B | C |
| ATOM | 3949 | C | ILE H | 64 | 23.837 | 22.859 | 24.601 | 1.00 | 22.58 | B | C |
| ATOM | 3950 | O | ILE H | 64 | 24.450 | 23.577 | 23.811 | 1.00 | 23.07 | B | O |
| ATOM | 3951 | N | LYS H | 65 | 24.104 | 22.827 | 25.903 | 1.00 | 24.21 | B | N |
| ATOM | 3952 | CA | LYS H | 65 | 25.356 | 23.354 | 26.435 | 1.00 | 25.86 | B | C |
| ATOM | 3953 | CB | LYS H | 65 | 25.109 | 24.202 | 27.686 | 1.00 | 25.90 | B | C |
| ATOM | 3954 | CG | LYS H | 65 | 23.736 | 24.015 | 28.312 | 1.00 | 26.69 | B | C |
| ATOM | 3955 | CD | LYS H | 65 | 23.556 | 22.597 | 28.830 | 1.00 | 28.26 | B | C |
| ATOM | 3956 | CE | LYS H | 65 | 23.104 | 22.591 | 30.281 | 1.00 | 28.67 | B | C |
| ATOM | 3957 | NZ | LYS H | 65 | 23.397 | 21.291 | 30.946 | 1.00 | 28.95 | B | N |
| ATOM | 3958 | C | LYS H | 65 | 26.377 | 22.252 | 26.719 | 1.00 | 26.81 | B | C |
| ATOM | 3959 | O | LYS H | 65 | 26.297 | 21.561 | 27.736 | 1.00 | 26.72 | B | O |
| ATOM | 3960 | N | GLU H | 66 | 27.382 | 22.159 | 25.852 | 1.00 | 27.90 | B | N |
| ATOM | 3961 | CA | GLU H | 66 | 28.276 | 21.001 | 25.793 | 1.00 | 28.82 | B | C |
| ATOM | 3962 | CB | GLU H | 66 | 29.031 | 20.991 | 24.457 | 1.00 | 28.88 | B | C |
| ATOM | 3963 | CG | GLU H | 66 | 30.531 | 20.737 | 24.572 | 1.00 | 30.33 | B | C |
| ATOM | 3964 | CD | GLU H | 66 | 31.320 | 21.283 | 23.391 | 1.00 | 33.17 | B | C |
| ATOM | 3965 | OE1 | GLU H | 66 | 32.446 | 20.796 | 23.151 | 1.00 | 35.05 | B | O |
| ATOM | 3966 | OE2 | GLU H | 66 | 30.864 | 22.268 | 22.775 | 1.00 | 33.14 | B | O |
| ATOM | 3967 | C | GLU H | 66 | 29.267 | 21.029 | 26.954 | 1.00 | 29.12 | B | C |
| ATOM | 3968 | O | GLU H | 66 | 29.751 | 22.097 | 27.329 | 1.00 | 29.28 | B | O |
| ATOM | 3969 | N | ASN H | 67 | 29.545 | 19.871 | 27.547 | 1.00 | 29.32 | B | N |
| ATOM | 3970 | CA | ASN H | 67 | 30.326 | 19.847 | 28.783 | 1.00 | 29.39 | B | C |
| ATOM | 3971 | CB | ASN H | 67 | 29.525 | 19.250 | 29.941 | 1.00 | 29.62 | B | C |
| ATOM | 3972 | CG | ASN H | 67 | 28.892 | 20.317 | 30.814 | 1.00 | 29.84 | B | C |
| ATOM | 3973 | OD1 | ASN H | 67 | 29.589 | 21.032 | 31.534 | 1.00 | 29.93 | B | O |
| ATOM | 3974 | ND2 | ASN H | 67 | 27.597 | 20.540 | 30.625 | 1.00 | 29.22 | B | N |
| ATOM | 3975 | C | ASN H | 67 | 31.745 | 19.271 | 28.698 | 1.00 | 29.15 | B | C |
| ATOM | 3976 | O | ASN H | 67 | 31.977 | 18.111 | 29.044 | 1.00 | 29.06 | B | O |
| ATOM | 3977 | N | LYS H | 68 | 32.625 | 20.020 | 28.036 | 1.00 | 28.87 | B | N |
| ATOM | 3978 | CA | LYS H | 68 | 34.033 | 20.170 | 28.428 | 1.00 | 28.78 | B | C |
| ATOM | 3979 | CB | LYS H | 68 | 34.334 | 21.625 | 28.806 | 1.00 | 28.89 | B | C |
| ATOM | 3980 | CG | LYS H | 68 | 33.742 | 22.653 | 27.846 | 1.00 | 29.26 | B | C |
| ATOM | 3981 | CD | LYS H | 68 | 33.794 | 24.057 | 28.427 | 1.00 | 29.68 | B | C |
| ATOM | 3982 | CE | LYS H | 68 | 35.058 | 24.267 | 29.243 | 1.00 | 29.46 | B | C |
| ATOM | 3983 | NZ | LYS H | 68 | 35.195 | 25.675 | 29.705 | 1.00 | 29.58 | B | N |
| ATOM | 3984 | C | LYS H | 68 | 34.543 | 19.202 | 29.509 | 1.00 | 28.60 | B | C |
| ATOM | 3985 | O | LYS H | 68 | 34.879 | 19.622 | 30.618 | 1.00 | 28.64 | B | O |
| ATOM | 3986 | N | CYS H | 69 | 34.618 | 17.912 | 29.162 | 1.00 | 28.09 | B | N |
| ATOM | 3987 | CA | CYS H | 69 | 35.404 | 16.889 | 29.895 | 1.00 | 27.63 | B | C |
| ATOM | 3988 | CB | CYS H | 69 | 34.502 | 15.694 | 30.287 | 1.00 | 27.07 | B | C |
| ATOM | 3989 | SG | CYS H | 69 | 34.515 | 14.237 | 29.134 | 1.00 | 28.01 | B | S |
| ATOM | 3990 | C | CYS H | 69 | 36.550 | 16.388 | 29.003 | 1.00 | 27.69 | B | C |
| ATOM | 3991 | O | CYS H | 69 | 36.474 | 16.529 | 27.785 | 1.00 | 27.41 | B | O |
| ATOM | 3992 | N | ASN H | 70 | 37.563 | 15.732 | 29.576 | 1.00 | 28.06 | B | N |
| ATOM | 3993 | CA | ASN H | 70 | 38.498 | 14.929 | 28.760 | 1.00 | 28.87 | B | C |
| ATOM | 3994 | CB | ASN H | 70 | 39.930 | 15.507 | 28.746 | 1.00 | 30.52 | B | C |
| ATOM | 3995 | CG | ASN H | 70 | 40.024 | 16.921 | 29.323 | 1.00 | 39.60 | B | C |
| ATOM | 3996 | OD1 | ASN H | 70 | 39.691 | 17.905 | 28.661 | 1.00 | 44.97 | B | O |
| ATOM | 3997 | ND2 | ASN H | 70 | 40.562 | 17.019 | 30.542 | 1.00 | 51.04 | B | N |
| ATOM | 3998 | C | ASN H | 70 | 38.528 | 13.423 | 29.067 | 1.00 | 26.97 | B | C |
| ATOM | 3999 | O | ASN H | 70 | 39.060 | 13.009 | 30.096 | 1.00 | 26.50 | B | O |
| ATOM | 4000 | N | GLY H | 71 | 38.111 | 12.611 | 28.096 | 1.00 | 25.46 | B | N |
| ATOM | 4001 | CA | GLY H | 71 | 38.117 | 11.148 | 28.237 | 1.00 | 23.67 | B | C |
| ATOM | 4002 | C | GLY H | 71 | 39.357 | 10.485 | 27.657 | 1.00 | 22.81 | B | C |
| ATOM | 4003 | O | GLY H | 71 | 40.279 | 11.169 | 27.211 | 1.00 | 22.79 | B | O |
| ATOM | 4004 | N | THR H | 72 | 39.400 | 9.155 | 27.687 | 1.00 | 21.93 | B | N |
| ATOM | 4005 | CA | THR H | 72 | 40.517 | 8.418 | 27.100 | 1.00 | 21.30 | B | C |
| ATOM | 4006 | CB | THR H | 72 | 40.594 | 6.975 | 27.644 | 1.00 | 21.12 | B | C |
| ATOM | 4007 | OG1 | THR H | 72 | 39.700 | 6.131 | 26.909 | 1.00 | 19.87 | B | O |
| ATOM | 4008 | CG2 | THR H | 72 | 40.210 | 6.947 | 29.115 | 1.00 | 20.76 | B | C |
| ATOM | 4009 | C | THR H | 72 | 40.436 | 8.407 | 25.571 | 1.00 | 21.50 | B | C |
| ATOM | 4010 | O | THR H | 72 | 39.458 | 7.924 | 24.999 | 1.00 | 21.85 | B | O |
| ATOM | 4011 | N | ASP H | 73 | 41.391 | 9.077 | 24.927 | 1.00 | 21.43 | B | N |
| ATOM | 4012 | CA | ASP H | 73 | 41.246 | 9.509 | 23.534 | 1.00 | 21.46 | B | C |
| ATOM | 4013 | CB | ASP H | 73 | 41.137 | 8.304 | 22.598 | 1.00 | 21.69 | B | C |
| ATOM | 4014 | CG | ASP H | 73 | 40.965 | 8.708 | 21.150 | 1.00 | 22.46 | B | C |
| ATOM | 4015 | OD1 | ASP H | 73 | 40.678 | 9.896 | 20.894 | 1.00 | 23.01 | B | O |
| ATOM | 4016 | OD2 | ASP H | 73 | 41.102 | 7.835 | 20.269 | 1.00 | 23.09 | B | O |
| ATOM | 4017 | C | ASP H | 73 | 40.060 | 10.451 | 23.324 | 1.00 | 20.96 | B | C |
| ATOM | 4018 | O | ASP H | 73 | 38.909 | 10.066 | 23.530 | 1.00 | 20.61 | B | O |
| ATOM | 4019 | N | ALA H | 74 | 40.342 | 11.642 | 22.802 | 1.00 | 20.42 | B | N |
| ATOM | 4020 | CA | ALA H | 74 | 39.342 | 12.698 | 22.730 | 1.00 | 20.38 | B | C |
| ATOM | 4021 | CB | ALA H | 74 | 39.707 | 13.839 | 23.670 | 1.00 | 20.11 | B | C |
| ATOM | 4022 | C | ALA H | 74 | 39.133 | 13.223 | 21.310 | 1.00 | 20.45 | B | C |
| ATOM | 4023 | O | ALA H | 74 | 38.545 | 14.286 | 21.121 | 1.00 | 20.46 | B | O |
| ATOM | 4024 | N | LYS H | 75 | 39.659 | 12.513 | 20.318 | 1.00 | 20.67 | B | N |
| ATOM | 4025 | CA | LYS H | 75 | 39.416 | 12.887 | 18.929 | 1.00 | 20.99 | B | C |
| ATOM | 4026 | CB | LYS H | 75 | 40.195 | 11.977 | 17.978 | 1.00 | 21.20 | B | C |
| ATOM | 4027 | CG | LYS H | 75 | 41.704 | 12.110 | 18.091 | 1.00 | 22.02 | B | C |
| ATOM | 4028 | CD | LYS H | 75 | 42.349 | 10.782 | 18.451 | 1.00 | 23.66 | B | C |
| ATOM | 4029 | CE | LYS H | 75 | 43.483 | 10.971 | 19.447 | 1.00 | 24.93 | B | C |
| ATOM | 4030 | NZ | LYS H | 75 | 43.559 | 9.859 | 20.434 | 1.00 | 25.55 | B | N |
| ATOM | 4031 | C | LYS H | 75 | 37.925 | 12.790 | 18.645 | 1.00 | 20.92 | B | C |
| ATOM | 4032 | O | LYS H | 75 | 37.397 | 13.474 | 17.768 | 1.00 | 20.92 | B | O |
| ATOM | 4033 | N | VAL H | 76 | 37.265 | 11.904 | 19.382 | 1.00 | 20.63 | B | N |
| ATOM | 4034 | CA | VAL H | 76 | 35.813 | 11.844 | 19.414 | 1.00 | 20.36 | B | C |
| ATOM | 4035 | CB | VAL H | 76 | 35.326 | 10.521 | 20.029 | 1.00 | 20.25 | B | C |
| ATOM | 4036 | CG1 | VAL H | 76 | 33.817 | 10.537 | 20.157 | 1.00 | 20.82 | B | C |
| ATOM | 4037 | CG2 | VAL H | 76 | 35.769 | 9.341 | 19.175 | 1.00 | 20.09 | B | C |
| ATOM | 4038 | C | VAL H | 76 | 35.234 | 13.006 | 20.213 | 1.00 | 20.29 | B | C |
| ATOM | 4039 | O | VAL H | 76 | 35.629 | 13.244 | 21.356 | 1.00 | 20.21 | B | O |
| ATOM | 4040 | N | LYS H | 77 | 34.284 | 13.715 | 19.613 | 1.00 | 20.17 | B | N |
| ATOM | 4041 | CA | LYS H | 77 | 33.726 | 14.916 | 20.224 | 1.00 | 20.17 | B | C |
| ATOM | 4042 | CB | LYS H | 77 | 34.593 | 16.136 | 19.904 | 1.00 | 20.62 | B | C |
| ATOM | 4043 | CG | LYS H | 77 | 35.797 | 16.307 | 20.825 | 1.00 | 23.31 | B | C |
| ATOM | 4044 | CD | LYS H | 77 | 36.952 | 16.989 | 20.103 | 1.00 | 26.74 | B | C |
| ATOM | 4045 | CE | LYS H | 77 | 38.196 | 17.046 | 20.971 | 1.00 | 28.32 | B | C |
| ATOM | 4046 | NZ | LYS H | 77 | 39.203 | 18.004 | 20.436 | 1.00 | 29.66 | B | N |
| ATOM | 4047 | C | LYS H | 77 | 32.294 | 15.141 | 19.759 | 1.00 | 19.45 | B | C |
| ATOM | 4048 | O | LYS H | 77 | 31.971 | 16.170 | 19.165 | 1.00 | 19.36 | B | O |
| ATOM | 4049 | N | LEU H | 78 | 31.441 | 14.162 | 20.033 | 1.00 | 18.70 | B | N |
| ATOM | 4050 | CA | LEU H | 78 | 30.155 | 14.056 | 19.365 | 1.00 | 17.98 | B | C |
| ATOM | 4051 | CB | LEU H | 78 | 29.356 | 12.892 | 19.945 | 1.00 | 17.72 | B | C |
| ATOM | 4052 | CG | LEU H | 78 | 30.168 | 11.596 | 20.034 | 1.00 | 17.51 | B | C |
| ATOM | 4053 | CD1 | LEU H | 78 | 29.321 | 10.446 | 20.554 | 1.00 | 17.41 | B | C |
| ATOM | 4054 | CD2 | LEU H | 78 | 30.802 | 11.249 | 18.690 | 1.00 | 17.62 | B | C |
| ATOM | 4055 | C | LEU H | 78 | 29.354 | 15.355 | 19.422 | 1.00 | 17.77 | B | C |
| ATOM | 4056 | O | LEU H | 78 | 28.996 | 15.911 | 18.384 | 1.00 | 17.89 | B | O |
| ATOM | 4057 | N | ILE H | 79 | 29.145 | 15.882 | 20.624 | 1.00 | 17.29 | B | N |
| ATOM | 4058 | CA | ILE H | 79 | 28.336 | 17.086 | 20.778 | 1.00 | 17.00 | B | C |
| ATOM | 4059 | CB | ILE H | 79 | 28.194 | 17.527 | 22.256 | 1.00 | 16.96 | B | C |
| ATOM | 4060 | CG1 | ILE H | 79 | 27.588 | 16.401 | 23.098 | 1.00 | 17.21 | B | C |
| ATOM | 4061 | CD1 | ILE H | 79 | 27.086 | 16.855 | 24.459 | 1.00 | 17.51 | B | C |
| ATOM | 4062 | CG2 | ILE H | 79 | 27.307 | 18.762 | 22.359 | 1.00 | 17.30 | B | C |
| ATOM | 4063 | C | ILE H | 79 | 28.854 | 18.238 | 19.911 | 1.00 | 16.83 | B | C |
| ATOM | 4064 | O | ILE H | 79 | 28.119 | 18.753 | 19.065 | 1.00 | 17.02 | B | O |
| ATOM | 4065 | N | LYS H | 80 | 30.124 | 18.571 | 19.800 | 1.00 | 16.55 | B | N |
| ATOM | 4066 | CA | LYS H | 80 | 30.366 | 19.684 | 18.841 | 1.00 | 16.38 | B | C |
| ATOM | 4067 | CB | LYS H | 80 | 31.861 | 20.021 | 18.833 | 1.00 | 16.73 | B | C |
| ATOM | 4068 | CG | LYS H | 80 | 32.255 | 21.150 | 17.895 | 1.00 | 17.99 | B | C |
| ATOM | 4069 | CD | LYS H | 80 | 32.343 | 22.475 | 18.640 | 1.00 | 20.71 | B | C |
| ATOM | 4070 | CE | LYS H | 80 | 32.566 | 23.632 | 17.681 | 1.00 | 22.92 | B | C |
| ATOM | 4071 | NZ | LYS H | 80 | 31.339 | 23.929 | 16.886 | 1.00 | 24.75 | B | N |
| ATOM | 4072 | C | LYS H | 80 | 29.884 | 19.466 | 17.349 | 1.00 | 15.79 | B | C |
| ATOM | 4073 | O | LYS H | 80 | 29.216 | 20.348 | 16.717 | 1.00 | 15.32 | B | O |
| ATOM | 4074 | N | GLN H | 81 | 30.177 | 18.286 | 16.806 | 1.00 | 15.48 | B | N |
| ATOM | 4075 | CA | GLN H | 81 | 29.878 | 17.961 | 15.401 | 1.00 | 15.33 | B | C |
| ATOM | 4076 | CB | GLN H | 81 | 30.465 | 16.593 | 15.049 | 1.00 | 15.70 | B | C |
| ATOM | 4077 | CG | GLN H | 81 | 31.870 | 16.364 | 15.565 | 1.00 | 17.88 | B | C |
| ATOM | 4078 | CD | GLN H | 81 | 32.301 | 14.917 | 15.435 | 1.00 | 20.46 | B | C |
| ATOM | 4079 | OE1 | GLN H | 81 | 31.670 | 14.133 | 14.730 | 1.00 | 21.59 | B | O |
| ATOM | 4080 | NE2 | GLN H | 81 | 33.378 | 14.554 | 16.123 | 1.00 | 20.26 | B | N |
| ATOM | 4081 | C | GLN H | 81 | 28.388 | 17.966 | 15.047 | 1.00 | 14.63 | B | C |
| ATOM | 4082 | O | GLN H | 81 | 27.972 | 18.429 | 13.962 | 1.00 | 14.60 | B | O |
| ATOM | 4083 | N | GLU H | 82 | 27.589 | 17.434 | 15.964 | 1.00 | 13.81 | B | N |
| ATOM | 4084 | CA | GLU H | 82 | 26.157 | 17.373 | 15.759 | 1.00 | 13.37 | B | C |
| ATOM | 4085 | CB | GLU H | 82 | 25.479 | 16.677 | 16.938 | 1.00 | 13.20 | B | C |
| ATOM | 4086 | CG | GLU H | 82 | 25.315 | 15.176 | 16.781 | 1.00 | 15.04 | B | C |
| ATOM | 4087 | CD | GLU H | 82 | 24.344 | 14.824 | 15.669 | 1.00 | 17.51 | B | C |
| ATOM | 4088 | OE1 | GLU H | 82 | 23.130 | 15.074 | 15.841 | 1.00 | 18.22 | B | O |
| ATOM | 4089 | OE2 | GLU H | 82 | 24.789 | 14.286 | 14.632 | 1.00 | 17.87 | B | O |
| ATOM | 4090 | C | GLU H | 82 | 25.650 | 18.794 | 15.638 | 1.00 | 12.99 | B | C |
| ATOM | 4091 | O | GLU H | 82 | 24.834 | 19.102 | 14.767 | 1.00 | 12.89 | B | O |
| ATOM | 4092 | N | LEU H | 83 | 26.174 | 19.677 | 16.482 | 1.00 | 12.89 | B | N |
| ATOM | 4093 | CA | LEU H | 83 | 25.727 | 21.057 | 16.454 | 1.00 | 13.08 | B | C |
| ATOM | 4094 | CB | LEU H | 83 | 26.419 | 21.865 | 17.552 | 1.00 | 13.26 | B | C |
| ATOM | 4095 | CG | LEU H | 83 | 26.057 | 21.498 | 18.994 | 1.00 | 14.18 | B | C |
| ATOM | 4096 | CD1 | LEU H | 83 | 26.535 | 22.566 | 19.968 | 1.00 | 15.11 | B | C |
| ATOM | 4097 | CD2 | LEU H | 83 | 24.558 | 21.282 | 19.117 | 1.00 | 14.79 | B | C |
| ATOM | 4098 | C | LEU H | 83 | 26.032 | 21.656 | 15.092 | 1.00 | 13.18 | B | C |
| ATOM | 4099 | O | LEU H | 83 | 25.150 | 22.261 | 14.459 | 1.00 | 13.05 | B | O |
| ATOM | 4100 | N | ASP H | 84 | 27.239 | 21.416 | 14.581 | 1.00 | 13.56 | B | N |
| ATOM | 4101 | CA | ASP H | 84 | 27.527 | 21.999 | 13.258 | 1.00 | 14.15 | B | C |
| ATOM | 4102 | CB | ASP H | 84 | 28.982 | 21.729 | 12.856 | 1.00 | 14.80 | B | C |
| ATOM | 4103 | CG | ASP H | 84 | 29.949 | 21.873 | 14.018 | 1.00 | 18.26 | B | C |
| ATOM | 4104 | OD1 | ASP H | 84 | 30.237 | 23.025 | 14.417 | 1.00 | 21.11 | B | O |
| ATOM | 4105 | OD2 | ASP H | 84 | 30.439 | 20.837 | 14.522 | 1.00 | 22.12 | B | O |
| ATOM | 4106 | C | ASP H | 84 | 26.572 | 21.490 | 12.150 | 1.00 | 13.56 | B | C |
| ATOM | 4107 | O | ASP H | 84 | 26.023 | 22.286 | 11.326 | 1.00 | 13.70 | B | O |
| ATOM | 4108 | N | LYS H | 85 | 26.328 | 20.180 | 12.146 | 1.00 | 12.87 | B | N |
| ATOM | 4109 | CA | LYS H | 85 | 25.458 | 19.616 | 11.106 | 1.00 | 12.31 | B | C |
| ATOM | 4110 | CB | LYS H | 85 | 25.412 | 18.090 | 11.210 | 1.00 | 12.41 | B | C |
| ATOM | 4111 | CG | LYS H | 85 | 24.611 | 17.417 | 10.110 | 1.00 | 12.26 | B | C |
| ATOM | 4112 | CD | LYS H | 85 | 24.213 | 16.002 | 10.506 | 1.00 | 12.72 | B | C |
| ATOM | 4113 | CE | LYS H | 85 | 23.359 | 16.018 | 11.762 | 1.00 | 14.40 | B | C |
| ATOM | 4114 | NZ | LYS H | 85 | 22.929 | 14.654 | 12.180 | 1.00 | 16.45 | B | N |
| ATOM | 4115 | C | LYS H | 85 | 24.038 | 20.192 | 11.202 | 1.00 | 11.85 | B | C |
| ATOM | 4116 | O | LYS H | 85 | 23.384 | 20.522 | 10.179 | 1.00 | 11.66 | B | O |
| ATOM | 4117 | N | TYR H | 86 | 23.571 | 20.318 | 12.442 | 1.00 | 11.32 | B | N |
| ATOM | 4118 | CA | TYR H | 86 | 22.232 | 20.825 | 12.683 | 1.00 | 11.07 | B | C |
| ATOM | 4119 | CB | TYR H | 86 | 21.875 | 20.784 | 14.167 | 1.00 | 11.12 | B | C |
| ATOM | 4120 | CG | TYR H | 86 | 20.899 | 21.871 | 14.551 | 1.00 | 11.30 | B | C |
| ATOM | 4121 | CD1 | TYR H | 86 | 19.564 | 21.805 | 14.169 | 1.00 | 12.16 | B | C |
| ATOM | 4122 | CE1 | TYR H | 86 | 18.672 | 22.804 | 14.514 | 1.00 | 12.39 | B | C |
| ATOM | 4123 | CZ | TYR H | 86 | 19.106 | 23.886 | 15.243 | 1.00 | 12.62 | B | C |
| ATOM | 4124 | OH | TYR H | 86 | 18.218 | 24.880 | 15.587 | 1.00 | 12.72 | B | O |
| ATOM | 4125 | CE2 | TYR H | 86 | 20.428 | 23.980 | 15.632 | 1.00 | 11.38 | B | C |
| ATOM | 4126 | CD2 | TYR H | 86 | 21.315 | 22.979 | 15.284 | 1.00 | 11.05 | B | C |
| ATOM | 4127 | C | TYR H | 86 | 22.120 | 22.247 | 12.181 | 1.00 | 10.75 | B | C |
| ATOM | 4128 | O | TYR H | 86 | 21.114 | 22.622 | 11.573 | 1.00 | 10.45 | B | O |
| ATOM | 4129 | N | LYS H | 87 | 23.155 | 23.046 | 12.419 | 1.00 | 10.66 | B | N |
| ATOM | 4130 | CA | LYS H | 87 | 23.093 | 24.423 | 11.971 | 1.00 | 11.03 | B | C |
| ATOM | 4131 | CB | LYS H | 87 | 24.333 | 25.191 | 12.407 | 1.00 | 11.36 | B | C |
| ATOM | 4132 | CG | LYS H | 87 | 24.600 | 25.106 | 13.890 | 1.00 | 12.79 | B | C |
| ATOM | 4133 | CD | LYS H | 87 | 25.805 | 25.931 | 14.269 | 1.00 | 14.76 | B | C |
| ATOM | 4134 | CE | LYS H | 87 | 25.989 | 25.979 | 15.771 | 1.00 | 15.29 | B | C |
| ATOM | 4135 | NZ | LYS H | 87 | 27.137 | 26.849 | 16.138 | 1.00 | 16.27 | B | N |
| ATOM | 4136 | C | LYS H | 87 | 22.961 | 24.455 | 10.457 | 1.00 | 10.83 | B | C |
| ATOM | 4137 | O | LYS H | 87 | 22.122 | 25.189 | 9.920 | 1.00 | 11.11 | B | O |
| ATOM | 4138 | N | ASN H | 88 | 23.724 | 23.621 | 9.755 | 1.00 | 10.52 | B | N |
| ATOM | 4139 | CA | ASN H | 88 | 23.568 | 23.646 | 8.297 | 1.00 | 10.21 | B | C |
| ATOM | 4140 | CB | ASN H | 88 | 24.586 | 22.715 | 7.640 | 1.00 | 10.61 | B | C |
| ATOM | 4141 | CG | ASN H | 88 | 24.426 | 22.646 | 6.134 | 1.00 | 11.81 | B | C |
| ATOM | 4142 | OD1 | ASN H | 88 | 24.215 | 23.664 | 5.474 | 1.00 | 12.97 | B | O |
| ATOM | 4143 | ND2 | ASN H | 88 | 24.515 | 21.443 | 5.585 | 1.00 | 13.82 | B | N |
| ATOM | 4144 | C | ASN H | 88 | 22.142 | 23.279 | 7.825 | 1.00 | 9.42 | B | C |
| ATOM | 4145 | O | ASN H | 88 | 21.538 | 23.959 | 6.939 | 1.00 | 8.94 | B | O |
| ATOM | 4146 | N | ALA H | 89 | 21.576 | 22.237 | 8.435 | 1.00 | 8.77 | B | N |
| ATOM | 4147 | CA | ALA H | 89 | 20.225 | 21.834 | 8.022 | 1.00 | 8.21 | B | C |
| ATOM | 4148 | CB | ALA H | 89 | 19.811 | 20.551 | 8.725 | 1.00 | 7.99 | B | C |
| ATOM | 4149 | C | ALA H | 89 | 19.208 | 22.951 | 8.293 | 1.00 | 7.78 | B | C |
| ATOM | 4150 | O | ALA H | 89 | 18.307 | 23.249 | 7.472 | 1.00 | 7.88 | B | O |
| ATOM | 4151 | N | VAL H | 90 | 19.383 | 23.581 | 9.449 | 1.00 | 7.01 | B | N |
| ATOM | 4152 | CA | VAL H | 90 | 18.493 | 24.639 | 9.880 | 1.00 | 6.26 | B | C |
| ATOM | 4153 | CB | VAL H | 90 | 18.866 | 25.140 | 11.289 | 1.00 | 5.91 | B | C |
| ATOM | 4154 | CG1 | VAL H | 90 | 18.089 | 26.401 | 11.626 | 1.00 | 5.92 | B | C |
| ATOM | 4155 | CG2 | VAL H | 90 | 18.608 | 24.054 | 12.318 | 1.00 | 5.26 | B | C |
| ATOM | 4156 | C | VAL H | 90 | 18.565 | 25.785 | 8.894 | 1.00 | 6.25 | B | C |
| ATOM | 4157 | O | VAL H | 90 | 17.547 | 26.368 | 8.538 | 1.00 | 6.32 | B | O |
| ATOM | 4158 | N | THR H | 91 | 19.771 | 26.092 | 8.434 | 1.00 | 6.24 | B | N |
| ATOM | 4159 | CA | THR H | 91 | 19.956 | 27.171 | 7.478 | 1.00 | 6.68 | B | C |
| ATOM | 4160 | CB | THR H | 91 | 21.444 | 27.375 | 7.139 | 1.00 | 6.62 | B | C |
| ATOM | 4161 | OG1 | THR H | 91 | 22.183 | 27.602 | 8.345 | 1.00 | 7.19 | B | O |
| ATOM | 4162 | CG2 | THR H | 91 | 21.619 | 28.565 | 6.210 | 1.00 | 7.27 | B | C |
| ATOM | 4163 | C | THR H | 91 | 19.203 | 26.886 | 6.180 | 1.00 | 7.02 | B | C |
| ATOM | 4164 | O | THR H | 91 | 18.505 | 27.767 | 5.661 | 1.00 | 7.16 | B | O |
| ATOM | 4165 | N | GLU H | 92 | 19.285 | 25.654 | 5.679 | 1.00 | 7.31 | B | N |
| ATOM | 4166 | CA | GLU H | 92 | 18.529 | 25.358 | 4.446 | 1.00 | 7.81 | B | C |
| ATOM | 4167 | CB | GLU H | 92 | 18.838 | 23.939 | 3.965 | 1.00 | 8.23 | B | C |
| ATOM | 4168 | CG | GLU H | 92 | 20.139 | 23.800 | 3.192 | 1.00 | 10.91 | B | C |
| ATOM | 4169 | CD | GLU H | 92 | 20.104 | 24.541 | 1.870 | 1.00 | 14.95 | B | C |
| ATOM | 4170 | OE1 | GLU H | 92 | 19.434 | 24.052 | 0.934 | 1.00 | 16.14 | B | O |
| ATOM | 4171 | OE2 | GLU H | 92 | 20.751 | 25.605 | 1.762 | 1.00 | 16.53 | B | O |
| ATOM | 4172 | C | GLU H | 92 | 17.003 | 25.530 | 4.644 | 1.00 | 7.47 | B | C |
| ATOM | 4173 | O | GLU H | 92 | 16.248 | 26.099 | 3.776 | 1.00 | 7.32 | B | O |
| ATOM | 4174 | N | LEU H | 93 | 16.541 | 25.053 | 5.800 | 1.00 | 7.30 | B | N |
| ATOM | 4175 | CA | LEU H | 93 | 15.117 | 25.171 | 6.098 | 1.00 | 7.47 | B | C |
| ATOM | 4176 | CB | LEU H | 93 | 14.760 | 24.426 | 7.385 | 1.00 | 7.15 | B | C |
| ATOM | 4177 | CG | LEU H | 93 | 14.797 | 22.898 | 7.297 | 1.00 | 7.20 | B | C |
| ATOM | 4178 | CD1 | LEU H | 93 | 14.346 | 22.282 | 8.610 | 1.00 | 7.93 | B | C |
| ATOM | 4179 | CD2 | LEU H | 93 | 13.941 | 22.397 | 6.141 | 1.00 | 6.91 | B | C |
| ATOM | 4180 | C | LEU H | 93 | 14.686 | 26.639 | 6.179 | 1.00 | 7.89 | B | C |
| ATOM | 4181 | O | LEU H | 93 | 13.626 | 27.012 | 5.675 | 1.00 | 7.94 | B | O |
| ATOM | 4182 | N | GLN H | 94 | 15.527 | 27.464 | 6.797 | 1.00 | 8.31 | B | N |
| ATOM | 4183 | CA | GLN H | 94 | 15.259 | 28.893 | 6.952 | 1.00 | 9.05 | B | C |
| ATOM | 4184 | CB | GLN H | 94 | 16.325 | 29.554 | 7.831 | 1.00 | 9.18 | B | C |
| ATOM | 4185 | CG | GLN H | 94 | 16.331 | 29.049 | 9.271 | 1.00 | 10.79 | B | C |
| ATOM | 4186 | CD | GLN H | 94 | 17.231 | 29.868 | 10.179 | 1.00 | 13.82 | B | C |
| ATOM | 4187 | OE1 | GLN H | 94 | 17.866 | 30.828 | 9.741 | 1.00 | 14.98 | B | O |
| ATOM | 4188 | NE2 | GLN H | 94 | 17.289 | 29.493 | 11.452 | 1.00 | 15.25 | B | N |
| ATOM | 4189 | C | GLN H | 94 | 15.190 | 29.545 | 5.576 | 1.00 | 9.25 | B | C |
| ATOM | 4190 | O | GLN H | 94 | 14.389 | 30.446 | 5.329 | 1.00 | 9.43 | B | O |
| ATOM | 4191 | N | LEU H | 95 | 16.050 | 29.058 | 4.691 | 1.00 | 9.55 | B | N |
| ATOM | 4192 | CA | LEU H | 95 | 16.140 | 29.479 | 3.294 | 1.00 | 10.17 | B | C |
| ATOM | 4193 | CB | LEU H | 95 | 17.327 | 28.800 | 2.605 | 1.00 | 9.79 | B | C |
| ATOM | 4194 | CG | LEU H | 95 | 18.721 | 29.321 | 2.958 | 1.00 | 8.86 | B | C |
| ATOM | 4195 | CD1 | LEU H | 95 | 19.789 | 28.548 | 2.198 | 1.00 | 8.31 | B | C |
| ATOM | 4196 | CD2 | LEU H | 95 | 18.823 | 30.809 | 2.667 | 1.00 | 7.62 | B | C |
| ATOM | 4197 | C | LEU H | 95 | 14.865 | 29.250 | 2.467 | 1.00 | 11.22 | B | C |
| ATOM | 4198 | O | LEU H | 95 | 14.579 | 30.030 | 1.560 | 1.00 | 11.55 | B | O |
| ATOM | 4199 | N | LEU H | 96 | 14.105 | 28.190 | 2.748 | 1.00 | 12.40 | B | N |
| ATOM | 4200 | CA | LEU H | 96 | 12.870 | 27.931 | 1.929 | 1.00 | 13.66 | B | C |
| ATOM | 4201 | CB | LEU H | 96 | 12.338 | 26.527 | 2.247 | 1.00 | 13.38 | B | C |
| ATOM | 4202 | CG | LEU H | 96 | 13.163 | 25.359 | 1.709 | 1.00 | 13.32 | B | C |
| ATOM | 4203 | CD1 | LEU H | 96 | 12.554 | 24.028 | 2.122 | 1.00 | 12.84 | B | C |
| ATOM | 4204 | CD2 | LEU H | 96 | 13.281 | 25.453 | 0.197 | 1.00 | 13.38 | B | C |
| ATOM | 4205 | C | LEU H | 96 | 11.631 | 28.927 | 1.839 | 1.00 | 14.97 | B | C |
| ATOM | 4206 | O | LEU H | 96 | 10.836 | 28.843 | 0.903 | 1.00 | 15.21 | B | O |
| ATOM | 4207 | N | MET H | 97 | 11.500 | 29.832 | 2.808 | 1.00 | 16.74 | B | N |
| ATOM | 4208 | CA | MET H | 97 | 10.348 | 30.575 | 3.317 | 1.00 | 18.96 | B | C |
| ATOM | 4209 | CB | MET H | 97 | 10.619 | 31.113 | 4.726 | 1.00 | 19.12 | B | C |
| ATOM | 4210 | CG | MET H | 97 | 10.944 | 30.037 | 5.758 | 1.00 | 21.87 | B | C |
| ATOM | 4211 | SD | MET H | 97 | 9.537 | 28.994 | 6.201 | 1.00 | 27.46 | B | S |
| ATOM | 4212 | CE | MET H | 97 | 8.667 | 28.891 | 4.638 | 1.00 | 24.25 | B | C |
| ATOM | 4213 | C | MET H | 97 | 9.912 | 31.705 | 2.386 | 1.00 | 20.09 | B | C |
| ATOM | 4214 | O | MET H | 97 | 8.725 | 32.019 | 2.294 | 1.00 | 20.58 | B | O |
| ATOM | 4215 | N | GLN H | 98 | 10.867 | 32.274 | 1.658 | 1.00 | 21.33 | B | N |
| ATOM | 4216 | CA | GLN H | 98 | 10.593 | 33.429 | 0.809 | 1.00 | 22.21 | B | C |
| ATOM | 4217 | CB | GLN H | 98 | 11.279 | 34.679 | 1.364 | 1.00 | 22.53 | B | C |
| ATOM | 4218 | CG | GLN H | 98 | 11.165 | 34.831 | 2.872 | 1.00 | 23.85 | B | C |
| ATOM | 4219 | CD | GLN H | 98 | 12.329 | 34.200 | 3.612 | 1.00 | 25.21 | B | C |
| ATOM | 4220 | OE1 | GLN H | 98 | 12.539 | 32.989 | 3.544 | 1.00 | 24.47 | B | O |
| ATOM | 4221 | NE2 | GLN H | 98 | 13.058 | 35.012 | 4.369 | 1.00 | 24.85 | B | N |
| ATOM | 4222 | C | GLN H | 98 | 11.035 | 33.183 | −0.629 | 1.00 | 22.15 | B | C |
| ATOM | 4223 | O | GLN H | 98 | 11.448 | 32.080 | −0.982 | 1.00 | 21.77 | B | O |
| ATOM | 4224 | N | PHE H | 137 | 5.312 | 39.031 | −3.961 | 1.00 | 33.25 | B | N |
| ATOM | 4225 | CA | PHE H | 137 | 5.277 | 37.601 | −4.244 | 1.00 | 36.79 | B | C |
| ATOM | 4226 | CB | PHE H | 137 | 3.903 | 37.193 | −4.778 | 1.00 | 20.00 | B | C |
| ATOM | 4227 | CG | PHE H | 137 | 2.795 | 37.350 | −3.779 | 1.00 | 20.00 | B | C |
| ATOM | 4228 | CD1 | PHE H | 137 | 2.542 | 36.360 | −2.843 | 1.00 | 20.00 | B | C |
| ATOM | 4229 | CE1 | PHE H | 137 | 1.524 | 36.503 | −1.920 | 1.00 | 20.00 | B | C |
| ATOM | 4230 | CZ | PHE H | 137 | 0.751 | 37.645 | −1.920 | 1.00 | 20.00 | B | C |
| ATOM | 4231 | CE2 | PHE H | 137 | 1.003 | 38.646 | −2.836 | 1.00 | 20.00 | B | C |
| ATOM | 4232 | CD2 | PHE H | 137 | 2.015 | 38.493 | −3.764 | 1.00 | 20.00 | B | C |
| ATOM | 4233 | C | PHE H | 137 | 6.359 | 37.207 | −5.241 | 1.00 | 48.73 | B | C |
| ATOM | 4234 | O | PHE H | 137 | 6.595 | 36.022 | −5.477 | 1.00 | 49.28 | B | O |
| ATOM | 4235 | N | LEU H | 138 | 6.975 | 38.204 | −5.868 | 1.00 | 42.04 | B | N |
| ATOM | 4236 | CA | LEU H | 138 | 8.094 | 37.959 | −6.768 | 1.00 | 44.26 | B | C |
| ATOM | 4237 | CB | LEU H | 138 | 8.582 | 39.268 | −7.389 | 1.00 | 20.00 | B | C |
| ATOM | 4238 | CG | LEU H | 138 | 7.578 | 40.005 | −8.278 | 1.00 | 20.00 | B | C |
| ATOM | 4239 | CD1 | LEU H | 138 | 8.178 | 41.296 | −8.816 | 1.00 | 20.00 | B | C |
| ATOM | 4240 | CD2 | LEU H | 138 | 7.101 | 39.112 | −9.411 | 1.00 | 20.00 | B | C |
| ATOM | 4241 | C | LEU H | 138 | 9.234 | 37.274 | −6.025 | 1.00 | 43.68 | B | C |
| ATOM | 4242 | O | LEU H | 138 | 10.107 | 36.658 | −6.636 | 1.00 | 50.27 | B | O |
| ATOM | 4243 | N | GLY H | 139 | 9.188 | 37.343 | −4.698 | 1.00 | 38.56 | B | N |
| ATOM | 4244 | CA | GLY H | 139 | 10.191 | 36.699 | −3.860 | 1.00 | 47.99 | B | C |
| ATOM | 4245 | C | GLY H | 139 | 10.260 | 35.202 | −4.081 | 1.00 | 44.53 | B | C |
| ATOM | 4246 | O | GLY H | 139 | 11.284 | 34.575 | −3.815 | 1.00 | 32.21 | B | O |
| ATOM | 4247 | N | PHE H | 140 | 9.173 | 34.630 | −4.590 | 1.00 | 46.33 | B | N |
| ATOM | 4248 | CA | PHE H | 140 | 9.070 | 33.183 | −4.735 | 1.00 | 42.95 | B | C |
| ATOM | 4249 | CB | PHE H | 140 | 7.618 | 32.723 | −4.576 | 1.00 | 20.00 | B | C |
| ATOM | 4250 | CG | PHE H | 140 | 7.058 | 32.941 | −3.201 | 1.00 | 20.00 | B | C |
| ATOM | 4251 | CD1 | PHE H | 140 | 7.285 | 32.021 | −2.190 | 1.00 | 20.00 | B | C |
| ATOM | 4252 | CE1 | PHE H | 140 | 6.763 | 32.217 | −0.926 | 1.00 | 20.00 | B | C |
| ATOM | 4253 | CZ | PHE H | 140 | 5.992 | 33.332 | −0.664 | 1.00 | 20.00 | B | C |
| ATOM | 4254 | CE2 | PHE H | 140 | 5.747 | 34.249 | −1.667 | 1.00 | 20.00 | B | C |
| ATOM | 4255 | CD2 | PHE H | 140 | 6.271 | 34.047 | −2.929 | 1.00 | 20.00 | B | C |
| ATOM | 4256 | C | PHE H | 140 | 9.632 | 32.695 | −6.071 | 1.00 | 38.13 | B | C |
| ATOM | 4257 | O | PHE H | 140 | 9.693 | 31.490 | −6.322 | 1.00 | 57.33 | B | O |
| ATOM | 4258 | N | LEU H | 141 | 10.017 | 33.631 | −6.934 | 1.00 | 35.20 | B | N |
| ATOM | 4259 | CA | LEU H | 141 | 10.453 | 33.294 | −8.288 | 1.00 | 27.01 | B | C |
| ATOM | 4260 | CB | LEU H | 141 | 11.557 | 32.235 | −8.254 | 1.00 | 20.00 | B | C |
| ATOM | 4261 | CG | LEU H | 141 | 12.886 | 32.672 | −7.636 | 1.00 | 20.00 | B | C |
| ATOM | 4262 | CD1 | LEU H | 141 | 13.836 | 31.493 | −7.513 | 1.00 | 20.00 | B | C |
| ATOM | 4263 | CD2 | LEU H | 141 | 13.520 | 33.794 | −8.445 | 1.00 | 20.00 | B | C |
| ATOM | 4264 | C | LEU H | 141 | 9.287 | 32.812 | −9.144 | 1.00 | 35.79 | B | C |
| ATOM | 4265 | O | LEU H | 141 | 8.355 | 33.567 | −9.417 | 1.00 | 46.92 | B | O |
| ATOM | 4266 | N | LEU H | 142 | 9.342 | 31.555 | −9.570 | 1.00 | 38.67 | B | N |
| ATOM | 4267 | CA | LEU H | 142 | 8.195 | 30.927 | −10.207 | 1.00 | 33.84 | B | C |
| ATOM | 4268 | CB | LEU H | 142 | 8.610 | 30.262 | −11.517 | 1.00 | 20.00 | B | C |
| ATOM | 4269 | CG | LEU H | 142 | 9.069 | 31.205 | −12.629 | 1.00 | 20.00 | B | C |
| ATOM | 4270 | CD1 | LEU H | 142 | 9.474 | 30.415 | −13.865 | 1.00 | 20.00 | B | C |
| ATOM | 4271 | CD2 | LEU H | 142 | 7.974 | 32.209 | −12.964 | 1.00 | 20.00 | B | C |
| ATOM | 4272 | C | LEU H | 142 | 7.541 | 29.910 | −9.278 | 1.00 | 36.89 | B | C |
| ATOM | 4273 | O | LEU H | 142 | 8.215 | 29.038 | −8.729 | 1.00 | 45.42 | B | O |
| ATOM | 4274 | N | GLY H | 143 | 6.268 | 30.132 | −8.975 | 1.00 | 26.84 | B | N |
| ATOM | 4275 | CA | GLY H | 143 | 5.577 | 29.350 | −7.958 | 1.00 | 27.41 | B | C |
| ATOM | 4276 | C | GLY H | 143 | 4.243 | 28.849 | −8.463 | 1.00 | 29.08 | B | C |
| ATOM | 4277 | O | GLY H | 143 | 3.592 | 29.504 | −9.277 | 1.00 | 40.24 | B | O |
| ATOM | 4278 | N | VAL H | 144 | 3.848 | 27.665 | −8.010 | 1.00 | 30.17 | B | N |
| ATOM | 4279 | CA | VAL H | 144 | 2.550 | 27.121 | −8.373 | 1.00 | 34.15 | B | C |
| ATOM | 4280 | CB | VAL H | 144 | 2.497 | 25.596 | −8.195 | 1.00 | 20.00 | B | C |
| ATOM | 4281 | CG1 | VAL H | 144 | 1.073 | 25.102 | −8.370 | 1.00 | 20.00 | B | C |
| ATOM | 4282 | CG2 | VAL H | 144 | 3.426 | 24.913 | −9.187 | 1.00 | 20.00 | B | C |
| ATOM | 4283 | C | VAL H | 144 | 1.449 | 27.759 | −7.538 | 1.00 | 36.67 | B | C |
| ATOM | 4284 | O | VAL H | 144 | 1.723 | 28.490 | −6.587 | 1.00 | 42.49 | B | O |
| ATOM | 4285 | N | GLY H | 145 | 0.204 | 27.526 | −7.938 | 1.00 | 47.09 | B | N |
| ATOM | 4286 | CA | GLY H | 145 | −0.946 | 27.979 | −7.166 | 1.00 | 44.51 | B | C |
| ATOM | 4287 | C | GLY H | 145 | −0.857 | 27.562 | −5.712 | 1.00 | 44.94 | B | C |
| ATOM | 4288 | O | GLY H | 145 | −1.097 | 28.365 | −4.810 | 1.00 | 40.58 | B | O |
| ATOM | 4289 | N | SER H | 146 | −0.529 | 26.297 | −5.473 | 1.00 | 42.06 | B | N |
| ATOM | 4290 | CA | SER H | 146 | −0.433 | 25.788 | −4.109 | 1.00 | 47.39 | B | C |
| ATOM | 4291 | CB | SER H | 146 | −0.198 | 24.275 | −4.119 | 1.00 | 20.00 | B | C |
| ATOM | 4292 | OG | SER H | 146 | 1.004 | 23.951 | −4.796 | 1.00 | 20.00 | B | O |
| ATOM | 4293 | C | SER H | 146 | 0.665 | 26.482 | −3.307 | 1.00 | 51.48 | B | C |
| ATOM | 4294 | O | SER H | 146 | 0.481 | 26.809 | −2.131 | 1.00 | 47.45 | B | O |
| ATOM | 4295 | N | ALA H | 147 | 1.807 | 26.710 | −3.949 | 1.00 | 39.51 | B | N |
| ATOM | 4296 | CA | ALA H | 147 | 2.943 | 27.334 | −3.282 | 1.00 | 43.38 | B | C |
| ATOM | 4297 | CB | ALA H | 147 | 4.162 | 27.326 | −4.194 | 1.00 | 20.00 | B | C |
| ATOM | 4298 | C | ALA H | 147 | 2.633 | 28.754 | −2.821 | 1.00 | 47.39 | B | C |
| ATOM | 4299 | O | ALA H | 147 | 2.997 | 29.147 | −1.713 | 1.00 | 50.18 | B | O |
| ATOM | 4300 | N | ILE H | 148 | 1.952 | 29.520 | −3.668 | 1.00 | 44.67 | B | N |
| ATOM | 4301 | CA | ILE H | 148 | 1.602 | 30.892 | −3.323 | 1.00 | 43.56 | B | C |
| ATOM | 4302 | CB | ILE H | 148 | 0.921 | 31.615 | −4.499 | 1.00 | 20.00 | B | C |
| ATOM | 4303 | CG1 | ILE H | 148 | 1.854 | 31.657 | −5.711 | 1.00 | 20.00 | B | C |
| ATOM | 4304 | CD1 | ILE H | 148 | 1.259 | 32.353 | −6.916 | 1.00 | 20.00 | B | C |
| ATOM | 4305 | CG2 | ILE H | 148 | 0.504 | 33.020 | −4.092 | 1.00 | 20.00 | B | C |
| ATOM | 4306 | C | ILE H | 148 | 0.672 | 30.913 | −2.117 | 1.00 | 51.18 | B | C |
| ATOM | 4307 | O | ILE H | 148 | 0.830 | 31.730 | −1.208 | 1.00 | 39.17 | B | O |
| ATOM | 4308 | N | ALA H | 149 | −0.289 | 29.995 | −2.109 | 1.00 | 42.17 | B | N |
| ATOM | 4309 | CA | ALA H | 149 | −1.233 | 29.895 | −1.007 | 1.00 | 36.76 | B | C |
| ATOM | 4310 | CB | ALA H | 149 | −2.297 | 28.854 | −1.312 | 1.00 | 20.00 | B | C |
| ATOM | 4311 | C | ALA H | 149 | −0.490 | 29.541 | 0.272 | 1.00 | 45.19 | B | C |
| ATOM | 4312 | O | ALA H | 149 | −0.771 | 30.090 | 1.340 | 1.00 | 47.83 | B | O |
| ATOM | 4313 | N | SER H | 150 | 0.471 | 28.630 | 0.157 | 1.00 | 43.64 | B | N |
| ATOM | 4314 | CA | SER H | 150 | 1.250 | 28.218 | 1.315 | 1.00 | 43.46 | B | C |
| ATOM | 4315 | CB | SER H | 150 | 2.211 | 27.088 | 0.943 | 1.00 | 20.00 | B | C |
| ATOM | 4316 | OG | SER H | 150 | 3.113 | 27.499 | −0.069 | 1.00 | 20.00 | B | O |
| ATOM | 4317 | C | SER H | 150 | 2.024 | 29.408 | 1.865 | 1.00 | 44.80 | B | C |
| ATOM | 4318 | O | SER H | 150 | 2.095 | 29.610 | 3.077 | 1.00 | 41.55 | B | O |
| ATOM | 4319 | N | GLY H | 151 | 2.590 | 30.205 | 0.964 | 1.00 | 27.01 | B | N |
| ATOM | 4320 | CA | GLY H | 151 | 3.342 | 31.379 | 1.362 | 1.00 | 35.21 | B | C |
| ATOM | 4321 | C | GLY H | 151 | 2.454 | 32.375 | 2.078 | 1.00 | 42.40 | B | C |
| ATOM | 4322 | O | GLY H | 151 | 2.849 | 32.969 | 3.083 | 1.00 | 45.11 | B | O |
| ATOM | 4323 | N | VAL H | 152 | 1.241 | 32.551 | 1.563 | 1.00 | 40.20 | B | N |
| ATOM | 4324 | CA | VAL H | 152 | 0.293 | 33.471 | 2.174 | 1.00 | 46.73 | B | C |
| ATOM | 4325 | CB | VAL H | 152 | −1.000 | 33.585 | 1.346 | 1.00 | 20.00 | B | C |
| ATOM | 4326 | CG1 | VAL H | 152 | −1.970 | 34.551 | 2.009 | 1.00 | 20.00 | B | C |
| ATOM | 4327 | CG2 | VAL H | 152 | −0.682 | 34.029 | −0.073 | 1.00 | 20.00 | B | C |
| ATOM | 4328 | C | VAL H | 152 | −0.052 | 32.995 | 3.579 | 1.00 | 48.62 | B | C |
| ATOM | 4329 | O | VAL H | 152 | −0.133 | 33.792 | 4.517 | 1.00 | 49.32 | B | O |
| ATOM | 4330 | N | ALA H | 153 | −0.239 | 31.686 | 3.722 | 1.00 | 35.09 | B | N |
| ATOM | 4331 | CA | ALA H | 153 | −0.567 | 31.112 | 5.018 | 1.00 | 42.37 | B | C |
| ATOM | 4332 | CB | ALA H | 153 | −0.849 | 29.625 | 4.885 | 1.00 | 20.00 | B | C |
| ATOM | 4333 | C | ALA H | 153 | 0.578 | 31.353 | 5.992 | 1.00 | 37.79 | B | C |
| ATOM | 4334 | O | ALA H | 153 | 0.359 | 31.696 | 7.154 | 1.00 | 39.25 | B | O |
| ATOM | 4335 | N | VAL H | 154 | 1.802 | 31.189 | 5.502 | 1.00 | 38.88 | B | N |
| ATOM | 4336 | CA | VAL H | 154 | 2.982 | 31.394 | 6.328 | 1.00 | 35.53 | B | C |
| ATOM | 4337 | CB | VAL H | 154 | 4.274 | 31.042 | 5.568 | 1.00 | 20.00 | B | C |
| ATOM | 4338 | CG1 | VAL H | 154 | 5.489 | 31.267 | 6.454 | 1.00 | 20.00 | B | C |
| ATOM | 4339 | CG2 | VAL H | 154 | 4.223 | 29.605 | 5.074 | 1.00 | 20.00 | B | C |
| ATOM | 4340 | C | VAL H | 154 | 3.053 | 32.842 | 6.791 | 1.00 | 41.00 | B | C |
| ATOM | 4341 | O | VAL H | 154 | 3.380 | 33.118 | 7.945 | 1.00 | 50.08 | B | O |
| ATOM | 4342 | N | SER H | 155 | 2.742 | 33.766 | 5.888 | 1.00 | 37.82 | B | N |
| ATOM | 4343 | CA | SER H | 155 | 2.753 | 35.181 | 6.232 | 1.00 | 44.68 | B | C |
| ATOM | 4344 | CB | SER H | 155 | 2.496 | 36.038 | 4.991 | 1.00 | 20.00 | B | C |
| ATOM | 4345 | OG | SER H | 155 | 1.238 | 35.736 | 4.414 | 1.00 | 20.00 | B | O |
| ATOM | 4346 | C | SER H | 155 | 1.706 | 35.477 | 7.300 | 1.00 | 41.82 | B | C |
| ATOM | 4347 | O | SER H | 155 | 1.960 | 36.224 | 8.245 | 1.00 | 49.37 | B | O |
| ATOM | 4348 | N | LYS H | 156 | 0.528 | 34.880 | 7.144 | 1.00 | 36.99 | B | N |
| ATOM | 4349 | CA | LYS H | 156 | −0.559 | 35.059 | 8.102 | 1.00 | 35.11 | B | C |
| ATOM | 4350 | CB | LYS H | 156 | −1.843 | 34.411 | 7.579 | 1.00 | 20.00 | B | C |
| ATOM | 4351 | CG | LYS H | 156 | −2.334 | 34.983 | 6.259 | 1.00 | 20.00 | B | C |
| ATOM | 4352 | CD | LYS H | 156 | −3.611 | 34.297 | 5.801 | 1.00 | 20.00 | B | C |
| ATOM | 4353 | CE | LYS H | 156 | −4.102 | 34.869 | 4.481 | 1.00 | 20.00 | B | C |
| ATOM | 4354 | NZ | LYS H | 156 | −5.353 | 34.206 | 4.020 | 1.00 | 20.00 | B | N |
| ATOM | 4355 | C | LYS H | 156 | −0.210 | 34.493 | 9.476 | 1.00 | 35.98 | B | C |
| ATOM | 4356 | O | LYS H | 156 | −0.535 | 35.081 | 10.507 | 1.00 | 42.31 | B | O |
| ATOM | 4357 | N | VAL H | 157 | 0.448 | 33.339 | 9.470 | 1.00 | 37.88 | B | N |
| ATOM | 4358 | CA | VAL H | 157 | 0.794 | 32.617 | 10.689 | 1.00 | 50.94 | B | C |
| ATOM | 4359 | CB | VAL H | 157 | 1.143 | 31.145 | 10.396 | 1.00 | 20.00 | B | C |
| ATOM | 4360 | CG1 | VAL H | 157 | 1.572 | 30.439 | 11.672 | 1.00 | 20.00 | B | C |
| ATOM | 4361 | CG2 | VAL H | 157 | −0.043 | 30.435 | 9.762 | 1.00 | 20.00 | B | C |
| ATOM | 4362 | C | VAL H | 157 | 1.962 | 33.277 | 11.415 | 1.00 | 47.82 | B | C |
| ATOM | 4363 | O | VAL H | 157 | 2.296 | 32.905 | 12.541 | 1.00 | 56.87 | B | O |
| ATOM | 4364 | N | LEU H | 158 | 2.533 | 34.304 | 10.794 | 1.00 | 39.52 | B | N |
| ATOM | 4365 | CA | LEU H | 158 | 3.727 | 34.956 | 11.320 | 1.00 | 50.27 | B | C |
| ATOM | 4366 | CB | LEU H | 158 | 4.080 | 36.188 | 10.484 | 1.00 | 20.00 | B | C |
| ATOM | 4367 | CG | LEU H | 158 | 4.493 | 35.927 | 9.034 | 1.00 | 20.00 | B | C |
| ATOM | 4368 | CD1 | LEU H | 158 | 4.688 | 37.237 | 8.287 | 1.00 | 20.00 | B | C |
| ATOM | 4369 | CD2 | LEU H | 158 | 5.754 | 35.079 | 8.976 | 1.00 | 20.00 | B | C |
| ATOM | 4370 | C | LEU H | 158 | 3.589 | 35.334 | 12.794 | 1.00 | 54.47 | B | C |
| ATOM | 4371 | O | LEU H | 158 | 4.512 | 35.127 | 13.582 | 1.00 | 49.26 | B | O |
| ATOM | 4372 | N | HIS H | 159 | 2.448 | 35.754 | 13.309 | 1.00 | 59.45 | B | N |
| ATOM | 4373 | CA | HIS H | 159 | 2.473 | 36.028 | 14.762 | 1.00 | 49.11 | B | C |
| ATOM | 4374 | CB | HIS H | 159 | 1.136 | 36.614 | 15.221 | 1.00 | 20.00 | B | C |
| ATOM | 4375 | CG | HIS H | 159 | 0.780 | 37.903 | 14.548 | 1.00 | 20.00 | B | C |
| ATOM | 4376 | ND1 | HIS H | 159 | 1.149 | 39.132 | 15.051 | 1.00 | 20.00 | B | N |
| ATOM | 4377 | CE1 | HIS H | 159 | 0.699 | 40.085 | 14.254 | 1.00 | 20.00 | B | C |
| ATOM | 4378 | NE2 | HIS H | 159 | 0.052 | 39.519 | 13.252 | 1.00 | 20.00 | B | N |
| ATOM | 4379 | CD2 | HIS H | 159 | 0.087 | 38.155 | 13.412 | 1.00 | 20.00 | B | C |
| ATOM | 4380 | C | HIS H | 159 | 2.830 | 34.775 | 15.616 | 1.00 | 44.09 | B | C |
| ATOM | 4381 | O | HIS H | 159 | 3.627 | 34.822 | 16.600 | 1.00 | 42.13 | B | O |
| ATOM | 4382 | N | LEU H | 160 | 2.261 | 33.646 | 15.207 | 1.00 | 37.55 | B | N |
| ATOM | 4383 | CA | LEU H | 160 | 2.402 | 32.391 | 15.934 | 1.00 | 43.52 | B | C |
| ATOM | 4384 | CB | LEU H | 160 | 1.521 | 31.310 | 15.302 | 1.00 | 20.00 | B | C |
| ATOM | 4385 | CG | LEU H | 160 | 0.018 | 31.596 | 15.268 | 1.00 | 20.00 | B | C |
| ATOM | 4386 | CD1 | LEU H | 160 | −0.734 | 30.443 | 14.620 | 1.00 | 20.00 | B | C |
| ATOM | 4387 | CD2 | LEU H | 160 | −0.509 | 31.865 | 16.668 | 1.00 | 20.00 | B | C |
| ATOM | 4388 | C | LEU H | 160 | 3.839 | 31.887 | 16.044 | 1.00 | 47.22 | B | C |
| ATOM | 4389 | O | LEU H | 160 | 4.233 | 31.383 | 17.091 | 1.00 | 43.25 | B | O |
| ATOM | 4390 | N | GLU H | 161 | 4.620 | 32.011 | 14.976 | 1.00 | 40.88 | B | N |
| ATOM | 4391 | CA | GLU H | 161 | 5.996 | 31.524 | 15.008 | 1.00 | 40.61 | B | C |
| ATOM | 4392 | CB | GLU H | 161 | 6.644 | 31.658 | 13.628 | 1.00 | 20.00 | B | C |
| ATOM | 4393 | CG | GLU H | 161 | 5.936 | 30.878 | 12.532 | 1.00 | 20.00 | B | C |
| ATOM | 4394 | CD | GLU H | 161 | 6.606 | 31.031 | 11.181 | 1.00 | 20.00 | B | C |
| ATOM | 4395 | OE1 | GLU H | 161 | 6.113 | 30.437 | 10.200 | 1.00 | 20.00 | B | O |
| ATOM | 4396 | OE2 | GLU H | 161 | 7.628 | 31.745 | 11.101 | 1.00 | 20.00 | B | O |
| ATOM | 4397 | C | GLU H | 161 | 6.833 | 32.259 | 16.054 | 1.00 | 39.76 | B | C |
| ATOM | 4398 | O | GLU H | 161 | 7.606 | 31.642 | 16.795 | 1.00 | 44.02 | B | O |
| ATOM | 4399 | N | GLY H | 162 | 6.679 | 33.566 | 16.211 | 1.00 | 34.18 | B | N |
| ATOM | 4400 | CA | GLY H | 162 | 7.497 | 34.204 | 17.230 | 1.00 | 35.01 | B | C |
| ATOM | 4401 | C | GLY H | 162 | 7.170 | 33.593 | 18.586 | 1.00 | 34.86 | B | C |
| ATOM | 4402 | O | GLY H | 162 | 8.065 | 33.226 | 19.372 | 1.00 | 47.81 | B | O |
| ATOM | 4403 | N | GLU H | 163 | 5.875 | 33.431 | 18.838 | 1.00 | 40.78 | B | N |
| ATOM | 4404 | CA | GLU H | 163 | 5.403 | 32.797 | 20.058 | 1.00 | 37.20 | B | C |
| ATOM | 4405 | CB | GLU H | 163 | 3.877 | 32.847 | 20.137 | 1.00 | 20.00 | B | C |
| ATOM | 4406 | CG | GLU H | 163 | 3.301 | 34.254 | 20.134 | 1.00 | 20.00 | B | C |
| ATOM | 4407 | CD | GLU H | 163 | 1.788 | 34.264 | 20.214 | 1.00 | 20.00 | B | C |
| ATOM | 4408 | OE1 | GLU H | 163 | 1.199 | 35.365 | 20.219 | 1.00 | 20.00 | B | O |
| ATOM | 4409 | OE2 | GLU H | 163 | 1.186 | 33.171 | 20.273 | 1.00 | 20.00 | B | O |
| ATOM | 4410 | C | GLU H | 163 | 5.884 | 31.355 | 20.065 | 1.00 | 46.12 | B | C |
| ATOM | 4411 | O | GLU H | 163 | 6.295 | 30.824 | 21.098 | 1.00 | 58.66 | B | O |
| ATOM | 4412 | N | VAL H | 164 | 5.832 | 30.731 | 18.893 | 1.00 | 42.23 | B | N |
| ATOM | 4413 | CA | VAL H | 164 | 6.270 | 29.353 | 18.740 | 1.00 | 35.45 | B | C |
| ATOM | 4414 | CB | VAL H | 164 | 6.021 | 28.836 | 17.311 | 1.00 | 20.00 | B | C |
| ATOM | 4415 | CG1 | VAL H | 164 | 6.488 | 27.394 | 17.181 | 1.00 | 20.00 | B | C |
| ATOM | 4416 | CG2 | VAL H | 164 | 4.549 | 28.962 | 16.951 | 1.00 | 20.00 | B | C |
| ATOM | 4417 | C | VAL H | 164 | 7.753 | 29.241 | 19.061 | 1.00 | 46.32 | B | C |
| ATOM | 4418 | O | VAL H | 164 | 8.180 | 28.290 | 19.709 | 1.00 | 45.08 | B | O |
| ATOM | 4419 | N | ASN H | 165 | 8.536 | 30.217 | 18.611 | 1.00 | 40.82 | B | N |
| ATOM | 4420 | CA | ASN H | 165 | 9.966 | 30.227 | 18.891 | 1.00 | 46.48 | B | C |
| ATOM | 4421 | CB | ASN H | 165 | 10.653 | 31.372 | 18.145 | 1.00 | 20.00 | B | C |
| ATOM | 4422 | CG | ASN H | 165 | 10.488 | 31.270 | 16.642 | 1.00 | 20.00 | B | C |
| ATOM | 4423 | OD1 | ASN H | 165 | 10.982 | 32.113 | 15.893 | 1.00 | 20.00 | B | O |
| ATOM | 4424 | ND2 | ASN H | 165 | 9.792 | 30.232 | 16.192 | 1.00 | 20.00 | B | N |
| ATOM | 4425 | C | ASN H | 165 | 10.227 | 30.342 | 20.389 | 1.00 | 50.90 | B | C |
| ATOM | 4426 | O | ASN H | 165 | 11.099 | 29.655 | 20.941 | 1.00 | 47.11 | B | O |
| ATOM | 4427 | N | LYS H | 166 | 9.453 | 31.198 | 21.052 | 1.00 | 48.36 | B | N |
| ATOM | 4428 | CA | LYS H | 166 | 9.605 | 31.351 | 22.495 | 1.00 | 38.71 | B | C |
| ATOM | 4429 | CB | LYS H | 166 | 8.692 | 32.461 | 23.019 | 1.00 | 20.00 | B | C |
| ATOM | 4430 | CG | LYS H | 166 | 8.968 | 33.828 | 22.414 | 1.00 | 20.00 | B | C |
| ATOM | 4431 | CD | LYS H | 166 | 8.030 | 34.881 | 22.980 | 1.00 | 20.00 | B | C |
| ATOM | 4432 | CE | LYS H | 166 | 8.306 | 36.249 | 22.376 | 1.00 | 20.00 | B | C |
| ATOM | 4433 | NZ | LYS H | 166 | 7.396 | 37.292 | 22.923 | 1.00 | 20.00 | B | N |
| ATOM | 4434 | C | LYS H | 166 | 9.284 | 30.029 | 23.192 | 1.00 | 45.07 | B | C |
| ATOM | 4435 | O | LYS H | 166 | 9.977 | 29.609 | 24.131 | 1.00 | 56.09 | B | O |
| ATOM | 4436 | N | ILE H | 167 | 8.238 | 29.366 | 22.709 | 1.00 | 42.02 | B | N |
| ATOM | 4437 | CA | ILE H | 167 | 7.817 | 28.091 | 23.272 | 1.00 | 43.86 | B | C |
| ATOM | 4438 | CB | ILE H | 167 | 6.527 | 27.577 | 22.607 | 1.00 | 20.00 | B | C |
| ATOM | 4439 | CG1 | ILE H | 167 | 5.388 | 28.580 | 22.807 | 1.00 | 20.00 | B | C |
| ATOM | 4440 | CD1 | ILE H | 167 | 4.079 | 28.150 | 22.181 | 1.00 | 20.00 | B | C |
| ATOM | 4441 | CG2 | ILE H | 167 | 6.147 | 26.214 | 23.164 | 1.00 | 20.00 | B | C |
| ATOM | 4442 | C | ILE H | 167 | 8.916 | 27.053 | 23.099 | 1.00 | 49.02 | B | C |
| ATOM | 4443 | O | ILE H | 167 | 9.178 | 26.260 | 23.998 | 1.00 | 37.71 | B | O |
| ATOM | 4444 | N | LYS H | 168 | 9.557 | 27.068 | 21.935 | 1.00 | 33.87 | B | N |
| ATOM | 4445 | CA | LYS H | 168 | 10.644 | 26.146 | 21.640 | 1.00 | 35.11 | B | C |
| ATOM | 4446 | CB | LYS H | 168 | 11.103 | 26.304 | 20.189 | 1.00 | 20.00 | B | C |
| ATOM | 4447 | CG | LYS H | 168 | 10.014 | 26.040 | 19.162 | 1.00 | 20.00 | B | C |
| ATOM | 4448 | CD | LYS H | 168 | 10.535 | 26.220 | 17.746 | 1.00 | 20.00 | B | C |
| ATOM | 4449 | CE | LYS H | 168 | 9.445 | 25.959 | 16.719 | 1.00 | 20.00 | B | C |
| ATOM | 4450 | NZ | LYS H | 168 | 9.941 | 26.134 | 15.326 | 1.00 | 20.00 | B | N |
| ATOM | 4451 | C | LYS H | 168 | 11.813 | 26.380 | 22.588 | 1.00 | 39.27 | B | C |
| ATOM | 4452 | O | LYS H | 168 | 12.422 | 25.429 | 23.078 | 1.00 | 32.39 | B | O |
| ATOM | 4453 | N | SER H | 169 | 12.121 | 27.647 | 22.854 | 1.00 | 41.30 | B | N |
| ATOM | 4454 | CA | SER H | 169 | 13.203 | 27.963 | 23.783 | 1.00 | 43.23 | B | C |
| ATOM | 4455 | CB | SER H | 169 | 13.446 | 29.472 | 23.832 | 1.00 | 20.00 | B | C |
| ATOM | 4456 | OG | SER H | 169 | 12.284 | 30.162 | 24.259 | 1.00 | 20.00 | B | O |
| ATOM | 4457 | C | SER H | 169 | 12.865 | 27.436 | 25.178 | 1.00 | 41.44 | B | C |
| ATOM | 4458 | O | SER H | 169 | 13.723 | 26.871 | 25.883 | 1.00 | 44.82 | B | O |
| ATOM | 4459 | N | ALA H | 170 | 11.604 | 27.605 | 25.566 | 1.00 | 39.78 | B | N |
| ATOM | 4460 | CA | ALA H | 170 | 11.161 | 27.131 | 26.870 | 1.00 | 41.01 | B | C |
| ATOM | 4461 | CB | ALA H | 170 | 9.720 | 27.541 | 27.124 | 1.00 | 20.00 | B | C |
| ATOM | 4462 | C | ALA H | 170 | 11.308 | 25.615 | 26.948 | 1.00 | 46.04 | B | C |
| ATOM | 4463 | O | ALA H | 170 | 11.743 | 25.072 | 27.964 | 1.00 | 46.67 | B | O |
| ATOM | 4464 | N | LEU H | 171 | 10.955 | 24.940 | 25.859 | 1.00 | 34.26 | B | N |
| ATOM | 4465 | CA | LEU H | 171 | 11.050 | 23.489 | 25.782 | 1.00 | 37.69 | B | C |
| ATOM | 4466 | CB | LEU H | 171 | 10.458 | 22.982 | 24.466 | 1.00 | 20.00 | B | C |
| ATOM | 4467 | CG | LEU H | 171 | 8.984 | 23.305 | 24.216 | 1.00 | 20.00 | B | C |
| ATOM | 4468 | CD1 | LEU H | 171 | 8.529 | 22.743 | 22.878 | 1.00 | 20.00 | B | C |
| ATOM | 4469 | CD2 | LEU H | 171 | 8.118 | 22.773 | 25.348 | 1.00 | 20.00 | B | C |
| ATOM | 4470 | C | LEU H | 171 | 12.499 | 23.044 | 25.912 | 1.00 | 44.67 | B | C |
| ATOM | 4471 | O | LEU H | 171 | 12.797 | 22.054 | 26.579 | 1.00 | 34.27 | B | O |
| ATOM | 4472 | N | LEU H | 172 | 13.400 | 23.783 | 25.272 | 1.00 | 34.80 | B | N |
| ATOM | 4473 | CA | LEU H | 172 | 14.817 | 23.464 | 25.344 | 1.00 | 28.87 | B | C |
| ATOM | 4474 | CB | LEU H | 172 | 15.626 | 24.402 | 24.447 | 1.00 | 20.00 | B | C |
| ATOM | 4475 | CG | LEU H | 172 | 15.281 | 24.379 | 22.956 | 1.00 | 20.00 | B | C |
| ATOM | 4476 | CD1 | LEU H | 172 | 16.158 | 25.355 | 22.186 | 1.00 | 20.00 | B | C |
| ATOM | 4477 | CD2 | LEU H | 172 | 15.416 | 22.972 | 22.395 | 1.00 | 20.00 | B | C |
| ATOM | 4478 | C | LEU H | 172 | 15.291 | 23.577 | 26.786 | 1.00 | 29.90 | B | C |
| ATOM | 4479 | O | LEU H | 172 | 16.022 | 22.714 | 27.272 | 1.00 | 41.09 | B | O |
| ATOM | 4480 | N | SER H | 173 | 14.862 | 24.629 | 27.480 | 1.00 | 35.08 | B | N |
| ATOM | 4481 | CA | SER H | 173 | 15.184 | 24.754 | 28.902 | 1.00 | 35.53 | B | C |
| ATOM | 4482 | CB | SER H | 173 | 14.727 | 26.109 | 29.442 | 1.00 | 20.00 | B | C |
| ATOM | 4483 | OG | SER H | 173 | 13.328 | 26.272 | 29.294 | 1.00 | 20.00 | B | O |
| ATOM | 4484 | C | SER H | 173 | 14.506 | 23.618 | 29.673 | 1.00 | 35.07 | B | C |
| ATOM | 4485 | O | SER H | 173 | 15.056 | 23.037 | 30.609 | 1.00 | 51.28 | B | O |
| ATOM | 4486 | N | THR H | 174 | 13.286 | 23.341 | 29.234 | 1.00 | 46.35 | B | N |
| ATOM | 4487 | CA | THR H | 174 | 12.335 | 22.383 | 29.786 | 1.00 | 43.83 | B | C |
| ATOM | 4488 | CB | THR H | 174 | 10.885 | 22.764 | 29.429 | 1.00 | 20.00 | B | C |
| ATOM | 4489 | OG1 | THR H | 174 | 10.725 | 22.762 | 28.004 | 1.00 | 20.00 | B | O |
| ATOM | 4490 | CG2 | THR H | 174 | 10.547 | 24.144 | 29.971 | 1.00 | 20.00 | B | C |
| ATOM | 4491 | C | THR H | 174 | 12.616 | 20.979 | 29.263 | 1.00 | 52.54 | B | C |
| ATOM | 4492 | O | THR H | 174 | 12.523 | 20.726 | 28.063 | 1.00 | 64.10 | B | O |
| ATOM | 4493 | N | ASN H | 175 | 12.974 | 20.071 | 30.165 | 1.00 | 55.57 | B | N |
| ATOM | 4494 | CA | ASN H | 175 | 13.072 | 18.660 | 29.817 | 1.00 | 40.58 | B | C |
| ATOM | 4495 | CB | ASN H | 175 | 13.942 | 17.915 | 30.831 | 1.00 | 20.00 | B | C |
| ATOM | 4496 | CG | ASN H | 175 | 15.405 | 18.303 | 30.740 | 1.00 | 20.00 | B | C |
| ATOM | 4497 | OD1 | ASN H | 175 | 15.868 | 18.788 | 29.708 | 1.00 | 20.00 | B | O |
| ATOM | 4498 | ND2 | ASN H | 175 | 16.139 | 18.099 | 31.827 | 1.00 | 20.00 | B | N |
| ATOM | 4499 | C | ASN H | 175 | 11.695 | 18.009 | 29.714 | 1.00 | 43.65 | B | C |
| ATOM | 4500 | O | ASN H | 175 | 10.972 | 17.912 | 30.705 | 1.00 | 53.11 | B | O |
| ATOM | 4501 | N | LYS H | 176 | 11.296 | 17.666 | 28.491 | 1.00 | 44.89 | B | N |
| ATOM | 4502 | CA | LYS H | 176 | 9.986 | 17.068 | 28.250 | 1.00 | 46.42 | B | C |
| ATOM | 4503 | CB | LYS H | 176 | 8.873 | 18.052 | 28.618 | 1.00 | 20.00 | B | C |
| ATOM | 4504 | CG | LYS H | 176 | 8.873 | 18.468 | 30.088 | 1.00 | 20.00 | B | C |
| ATOM | 4505 | CD | LYS H | 176 | 7.844 | 19.557 | 30.362 | 1.00 | 20.00 | B | C |
| ATOM | 4506 | CE | LYS H | 176 | 7.773 | 19.904 | 31.841 | 1.00 | 20.00 | B | C |
| ATOM | 4507 | NZ | LYS H | 176 | 6.701 | 20.899 | 32.121 | 1.00 | 20.00 | B | N |
| ATOM | 4508 | C | LYS H | 176 | 9.829 | 16.596 | 26.806 | 1.00 | 45.00 | B | C |
| ATOM | 4509 | O | LYS H | 176 | 10.272 | 17.264 | 25.871 | 1.00 | 60.64 | B | O |
| ATOM | 4510 | N | ALA H | 177 | 9.245 | 15.415 | 26.634 | 1.00 | 46.01 | B | N |
| ATOM | 4511 | CA | ALA H | 177 | 8.914 | 14.913 | 25.306 | 1.00 | 43.65 | B | C |
| ATOM | 4512 | CB | ALA H | 177 | 8.530 | 13.442 | 25.374 | 1.00 | 20.00 | B | C |
| ATOM | 4513 | C | ALA H | 177 | 7.789 | 15.731 | 24.686 | 1.00 | 41.42 | B | C |
| ATOM | 4514 | O | ALA H | 177 | 7.894 | 16.184 | 23.547 | 1.00 | 45.37 | B | O |
| ATOM | 4515 | N | VAL H | 178 | 6.736 | 15.960 | 25.463 | 1.00 | 35.70 | B | N |
| ATOM | 4516 | CA | VAL H | 178 | 5.642 | 16.820 | 25.037 | 1.00 | 43.93 | B | C |
| ATOM | 4517 | CB | VAL H | 178 | 4.383 | 15.999 | 24.710 | 1.00 | 20.00 | B | C |
| ATOM | 4518 | CG1 | VAL H | 178 | 3.229 | 16.919 | 24.341 | 1.00 | 20.00 | B | C |
| ATOM | 4519 | CG2 | VAL H | 178 | 4.672 | 15.014 | 23.585 | 1.00 | 20.00 | B | C |
| ATOM | 4520 | C | VAL H | 178 | 5.313 | 17.836 | 26.124 | 1.00 | 34.16 | B | C |
| ATOM | 4521 | O | VAL H | 178 | 5.395 | 17.527 | 27.311 | 1.00 | 44.45 | B | O |
| ATOM | 4522 | N | VAL H | 179 | 5.028 | 19.069 | 25.717 | 1.00 | 44.87 | B | N |
| ATOM | 4523 | CA | VAL H | 179 | 4.818 | 20.155 | 26.667 | 1.00 | 38.47 | B | C |
| ATOM | 4524 | CB | VAL H | 179 | 6.150 | 20.766 | 27.131 | 1.00 | 20.00 | B | C |
| ATOM | 4525 | CG1 | VAL H | 179 | 5.908 | 21.808 | 28.221 | 1.00 | 20.00 | B | C |
| ATOM | 4526 | CG2 | VAL H | 179 | 7.097 | 19.678 | 27.609 | 1.00 | 20.00 | B | C |
| ATOM | 4527 | C | VAL H | 179 | 3.967 | 21.265 | 26.070 | 1.00 | 44.79 | B | C |
| ATOM | 4528 | O | VAL H | 179 | 4.226 | 21.727 | 24.959 | 1.00 | 51.88 | B | O |
| ATOM | 4529 | N | SER H | 180 | 3.025 | 21.765 | 26.862 | 1.00 | 45.23 | B | N |
| ATOM | 4530 | CA | SER H | 180 | 2.226 | 22.915 | 26.463 | 1.00 | 48.99 | B | C |
| ATOM | 4531 | CB | SER H | 180 | 0.976 | 23.033 | 27.340 | 1.00 | 20.00 | B | C |
| ATOM | 4532 | OG | SER H | 180 | 1.321 | 23.322 | 28.683 | 1.00 | 20.00 | B | O |
| ATOM | 4533 | C | SER H | 180 | 3.047 | 24.198 | 26.532 | 1.00 | 48.15 | B | C |
| ATOM | 4534 | O | SER H | 180 | 3.568 | 24.553 | 27.589 | 1.00 | 60.42 | B | O |
| ATOM | 4535 | N | LEU H | 181 | 3.261 | 24.821 | 25.378 | 1.00 | 42.42 | B | N |
| ATOM | 4536 | CA | LEU H | 181 | 3.872 | 26.143 | 25.325 | 1.00 | 43.22 | B | C |
| ATOM | 4537 | CB | LEU H | 181 | 4.252 | 26.498 | 23.888 | 1.00 | 20.00 | B | C |
| ATOM | 4538 | CG | LEU H | 181 | 5.279 | 25.577 | 23.233 | 1.00 | 20.00 | B | C |
| ATOM | 4539 | CD1 | LEU H | 181 | 5.583 | 26.043 | 21.818 | 1.00 | 20.00 | B | C |
| ATOM | 4540 | CD2 | LEU H | 181 | 6.541 | 25.519 | 24.073 | 1.00 | 20.00 | B | C |
| ATOM | 4541 | C | LEU H | 181 | 2.929 | 27.199 | 25.884 | 1.00 | 29.14 | B | C |
| ATOM | 4542 | O | LEU H | 181 | 1.891 | 27.494 | 25.291 | 1.00 | 43.42 | B | O |
| ATOM | 4543 | N | SER H | 182 | 3.271 | 27.735 | 27.051 | 1.00 | 41.83 | B | N |
| ATOM | 4544 | CA | SER H | 182 | 2.365 | 28.619 | 27.770 | 1.00 | 53.52 | B | C |
| ATOM | 4545 | CB | SER H | 182 | 2.034 | 28.053 | 29.152 | 1.00 | 20.00 | B | C |
| ATOM | 4546 | OG | SER H | 182 | 3.184 | 28.019 | 29.978 | 1.00 | 20.00 | B | O |
| ATOM | 4547 | C | SER H | 182 | 2.914 | 30.036 | 27.891 | 1.00 | 44.76 | B | C |
| ATOM | 4548 | O | SER H | 182 | 4.095 | 30.236 | 28.178 | 1.00 | 37.73 | B | O |
| ATOM | 4549 | N | ASN H | 183 | 2.072 | 31.008 | 27.556 | 1.00 | 47.84 | B | N |
| ATOM | 4550 | CA | ASN H | 183 | 2.258 | 32.384 | 27.996 | 1.00 | 59.45 | B | C |
| ATOM | 4551 | CB | ASN H | 183 | 2.304 | 33.316 | 26.782 | 1.00 | 20.00 | B | C |
| ATOM | 4552 | CG | ASN H | 183 | 3.512 | 33.066 | 25.897 | 1.00 | 20.00 | B | C |
| ATOM | 4553 | OD1 | ASN H | 183 | 4.574 | 32.671 | 26.379 | 1.00 | 20.00 | B | O |
| ATOM | 4554 | ND2 | ASN H | 183 | 3.384 | 33.386 | 24.615 | 1.00 | 20.00 | B | N |
| ATOM | 4555 | C | ASN H | 183 | 1.117 | 32.791 | 28.924 | 1.00 | 75.41 | B | C |
| ATOM | 4556 | O | ASN H | 183 | −0.008 | 32.318 | 28.769 | 1.00 | 102.31 | B | O |
| ATOM | 4557 | N | GLY H | 184 | 1.432 | 33.563 | 29.959 | 1.00 | 79.30 | B | N |
| ATOM | 4558 | CA | GLY H | 184 | 0.435 | 33.947 | 30.957 | 1.00 | 88.92 | B | C |
| ATOM | 4559 | C | GLY H | 184 | −0.954 | 33.397 | 30.674 | 1.00 | 101.85 | B | C |
| ATOM | 4560 | O | GLY H | 184 | −1.760 | 34.037 | 29.999 | 1.00 | 113.48 | B | O |
| ATOM | 4561 | N | VAL H | 185 | −1.218 | 32.190 | 31.167 | 1.00 | 86.84 | B | N |
| ATOM | 4562 | CA | VAL H | 185 | −2.573 | 31.636 | 31.236 | 1.00 | 81.80 | B | C |
| ATOM | 4563 | CB | VAL H | 185 | −3.558 | 32.579 | 31.958 | 1.00 | 20.00 | B | C |
| ATOM | 4564 | CG1 | VAL H | 185 | −4.974 | 32.022 | 31.887 | 1.00 | 20.00 | B | C |
| ATOM | 4565 | CG2 | VAL H | 185 | −3.135 | 32.783 | 33.405 | 1.00 | 20.00 | B | C |
| ATOM | 4566 | C | VAL H | 185 | −3.164 | 31.228 | 29.887 | 1.00 | 81.69 | B | C |
| ATOM | 4567 | O | VAL H | 185 | −4.347 | 30.900 | 29.801 | 1.00 | 99.84 | B | O |
| ATOM | 4568 | N | SER H | 186 | −2.336 | 31.193 | 28.848 | 1.00 | 70.63 | B | N |
| ATOM | 4569 | CA | SER H | 186 | −2.819 | 30.830 | 27.519 | 1.00 | 72.29 | B | C |
| ATOM | 4570 | CB | SER H | 186 | −3.084 | 32.081 | 26.679 | 1.00 | 20.00 | B | C |
| ATOM | 4571 | OG | SER H | 186 | −1.884 | 32.787 | 26.420 | 1.00 | 20.00 | B | O |
| ATOM | 4572 | C | SER H | 186 | −1.865 | 29.891 | 26.789 | 1.00 | 62.99 | B | C |
| ATOM | 4573 | O | SER H | 186 | −0.655 | 30.115 | 26.765 | 1.00 | 58.35 | B | O |
| ATOM | 4574 | N | VAL H | 187 | −2.422 | 28.851 | 26.177 | 1.00 | 66.49 | B | N |
| ATOM | 4575 | CA | VAL H | 187 | −1.627 | 27.877 | 25.438 | 1.00 | 57.36 | B | C |
| ATOM | 4576 | CB | VAL H | 187 | −2.284 | 26.484 | 25.457 | 1.00 | 20.00 | B | C |
| ATOM | 4577 | CG1 | VAL H | 187 | −1.504 | 25.516 | 24.583 | 1.00 | 20.00 | B | C |
| ATOM | 4578 | CG2 | VAL H | 187 | −2.383 | 25.963 | 26.883 | 1.00 | 20.00 | B | C |
| ATOM | 4579 | C | VAL H | 187 | −1.432 | 28.317 | 23.991 | 1.00 | 53.67 | B | C |
| ATOM | 4580 | O | VAL H | 187 | −2.401 | 28.531 | 23.262 | 1.00 | 50.10 | B | O |
| ATOM | 4581 | N | LEU H | 188 | −0.176 | 28.476 | 23.588 | 1.00 | 43.20 | B | N |
| ATOM | 4582 | CA | LEU H | 188 | 0.143 | 28.854 | 22.217 | 1.00 | 41.76 | B | C |
| ATOM | 4583 | CB | LEU H | 188 | 1.487 | 29.581 | 22.160 | 1.00 | 20.00 | B | C |
| ATOM | 4584 | CG | LEU H | 188 | 1.549 | 30.938 | 22.862 | 1.00 | 20.00 | B | C |
| ATOM | 4585 | CD1 | LEU H | 188 | 2.957 | 31.507 | 22.799 | 1.00 | 20.00 | B | C |
| ATOM | 4586 | CD2 | LEU H | 188 | 0.548 | 31.907 | 22.250 | 1.00 | 20.00 | B | C |
| ATOM | 4587 | C | LEU H | 188 | 0.166 | 27.634 | 21.304 | 1.00 | 44.75 | B | C |
| ATOM | 4588 | O | LEU H | 188 | −0.074 | 27.743 | 20.102 | 1.00 | 45.28 | B | O |
| ATOM | 4589 | N | THR H | 189 | 0.390 | 26.466 | 21.897 | 1.00 | 39.28 | B | N |
| ATOM | 4590 | CA | THR H | 189 | 0.552 | 25.235 | 21.133 | 1.00 | 36.51 | B | C |
| ATOM | 4591 | CB | THR H | 189 | 1.229 | 25.498 | 19.774 | 1.00 | 20.00 | B | C |
| ATOM | 4592 | OG1 | THR H | 189 | 2.554 | 26.001 | 19.986 | 1.00 | 20.00 | B | O |
| ATOM | 4593 | CG2 | THR H | 189 | 0.430 | 26.512 | 18.968 | 1.00 | 20.00 | B | C |
| ATOM | 4594 | C | THR H | 189 | 1.362 | 24.208 | 21.926 | 1.00 | 45.83 | B | C |
| ATOM | 4595 | O | THR H | 189 | 1.419 | 24.267 | 23.154 | 1.00 | 42.57 | B | O |
| ATOM | 4596 | N | SER H | 190 | 1.947 | 23.241 | 21.226 | 1.00 | 40.44 | B | N |
| ATOM | 4597 | CA | SER H | 190 | 2.691 | 22.171 | 21.881 | 1.00 | 41.82 | B | C |
| ATOM | 4598 | CB | SER H | 190 | 2.022 | 20.815 | 21.632 | 1.00 | 20.00 | B | C |
| ATOM | 4599 | OG | SER H | 190 | 2.225 | 20.376 | 20.300 | 1.00 | 20.00 | B | O |
| ATOM | 4600 | C | SER H | 190 | 4.152 | 22.141 | 21.437 | 1.00 | 33.81 | B | C |
| ATOM | 4601 | O | SER H | 190 | 4.473 | 22.505 | 20.306 | 1.00 | 49.61 | B | O |
| ATOM | 4602 | N | LYS H | 191 | 5.040 | 21.855 | 22.385 | 1.00 | 37.37 | B | N |
| ATOM | 4603 | CA | LYS H | 191 | 6.463 | 21.711 | 22.098 | 1.00 | 41.23 | B | C |
| ATOM | 4604 | CB | LYS H | 191 | 7.285 | 22.617 | 23.021 | 1.00 | 20.00 | B | C |
| ATOM | 4605 | CG | LYS H | 191 | 7.104 | 24.102 | 22.750 | 1.00 | 20.00 | B | C |
| ATOM | 4606 | CD | LYS H | 191 | 7.981 | 24.949 | 23.658 | 1.00 | 20.00 | B | C |
| ATOM | 4607 | CE | LYS H | 191 | 7.790 | 26.435 | 23.369 | 1.00 | 20.00 | B | C |
| ATOM | 4608 | NZ | LYS H | 191 | 8.364 | 27.306 | 24.434 | 1.00 | 20.00 | B | N |
| ATOM | 4609 | C | LYS H | 191 | 6.906 | 20.260 | 22.268 | 1.00 | 35.03 | B | C |
| ATOM | 4610 | O | LYS H | 191 | 6.828 | 19.710 | 23.365 | 1.00 | 40.06 | B | O |
| ATOM | 4611 | N | VAL H | 192 | 7.443 | 19.670 | 21.205 | 1.00 | 33.70 | B | N |
| ATOM | 4612 | CA | VAL H | 192 | 7.787 | 18.250 | 21.221 | 1.00 | 27.61 | B | C |
| ATOM | 4613 | CB | VAL H | 192 | 6.974 | 17.456 | 20.182 | 1.00 | 20.00 | B | C |
| ATOM | 4614 | CG1 | VAL H | 192 | 7.313 | 15.975 | 20.263 | 1.00 | 20.00 | B | C |
| ATOM | 4615 | CG2 | VAL H | 192 | 5.487 | 17.679 | 20.391 | 1.00 | 20.00 | B | C |
| ATOM | 4616 | C | VAL H | 192 | 9.277 | 18.011 | 20.985 | 1.00 | 31.74 | B | C |
| ATOM | 4617 | O | VAL H | 192 | 9.931 | 18.771 | 20.272 | 1.00 | 25.58 | B | O |
| ATOM | 4618 | N | LEU H | 193 | 9.792 | 16.921 | 21.550 | 1.00 | 28.88 | B | N |
| ATOM | 4619 | CA | LEU H | 193 | 11.190 | 16.530 | 21.360 | 1.00 | 34.36 | B | C |
| ATOM | 4620 | CB | LEU H | 193 | 12.061 | 17.069 | 22.505 | 1.00 | 20.00 | B | C |
| ATOM | 4621 | CG | LEU H | 193 | 12.332 | 18.577 | 22.614 | 1.00 | 20.00 | B | C |
| ATOM | 4622 | CD1 | LEU H | 193 | 12.987 | 18.914 | 23.949 | 1.00 | 20.00 | B | C |
| ATOM | 4623 | CD2 | LEU H | 193 | 13.194 | 19.076 | 21.459 | 1.00 | 20.00 | B | C |
| ATOM | 4624 | C | LEU H | 193 | 11.335 | 15.008 | 21.281 | 1.00 | 33.03 | B | C |
| ATOM | 4625 | O | LEU H | 193 | 11.373 | 14.331 | 22.310 | 1.00 | 44.93 | B | O |
| ATOM | 4626 | N | ASP H | 194 | 11.579 | 14.498 | 20.077 | 1.00 | 35.09 | B | N |
| ATOM | 4627 | CA | ASP H | 194 | 11.209 | 13.127 | 19.730 | 1.00 | 41.39 | B | C |
| ATOM | 4628 | CB | ASP H | 194 | 11.269 | 12.925 | 18.214 | 1.00 | 20.00 | B | C |
| ATOM | 4629 | CG | ASP H | 194 | 10.132 | 13.626 | 17.485 | 1.00 | 20.00 | B | C |
| ATOM | 4630 | OD1 | ASP H | 194 | 9.081 | 13.873 | 18.115 | 1.00 | 20.00 | B | O |
| ATOM | 4631 | OD2 | ASP H | 194 | 10.305 | 13.969 | 16.296 | 1.00 | 20.00 | B | O |
| ATOM | 4632 | C | ASP H | 194 | 12.091 | 12.100 | 20.433 | 1.00 | 38.04 | B | C |
| ATOM | 4633 | O | ASP H | 194 | 11.796 | 10.904 | 20.429 | 1.00 | 48.03 | B | O |
| ATOM | 4634 | N | LEU H | 195 | 13.174 | 12.576 | 21.039 | 1.00 | 43.64 | B | N |
| ATOM | 4635 | CA | LEU H | 195 | 14.206 | 11.690 | 21.555 | 1.00 | 35.97 | B | C |
| ATOM | 4636 | CB | LEU H | 195 | 15.532 | 11.935 | 20.832 | 1.00 | 20.00 | B | C |
| ATOM | 4637 | CG | LEU H | 195 | 15.563 | 11.586 | 19.340 | 1.00 | 20.00 | B | C |
| ATOM | 4638 | CD1 | LEU H | 195 | 16.947 | 11.852 | 18.750 | 1.00 | 20.00 | B | C |
| ATOM | 4639 | CD2 | LEU H | 195 | 15.118 | 10.144 | 19.081 | 1.00 | 20.00 | B | C |
| ATOM | 4640 | C | LEU H | 195 | 14.386 | 11.839 | 23.063 | 1.00 | 43.63 | B | C |
| ATOM | 4641 | O | LEU H | 195 | 15.068 | 11.036 | 23.699 | 1.00 | 34.71 | B | O |
| ATOM | 4642 | N | ASN H | 196 | 13.689 | 12.808 | 23.645 | 1.00 | 44.30 | B | N |
| ATOM | 4643 | CA | ASN H | 196 | 13.886 | 13.151 | 25.047 | 1.00 | 42.38 | B | C |
| ATOM | 4644 | CB | ASN H | 196 | 12.955 | 14.290 | 25.454 | 1.00 | 20.00 | B | C |
| ATOM | 4645 | CG | ASN H | 196 | 13.376 | 15.618 | 24.868 | 1.00 | 20.00 | B | C |
| ATOM | 4646 | OD1 | ASN H | 196 | 14.538 | 15.815 | 24.513 | 1.00 | 20.00 | B | O |
| ATOM | 4647 | ND2 | ASN H | 196 | 12.439 | 16.555 | 24.800 | 1.00 | 20.00 | B | N |
| ATOM | 4648 | C | ASN H | 196 | 13.721 | 11.972 | 26.000 | 1.00 | 43.66 | B | C |
| ATOM | 4649 | O | ASN H | 196 | 14.499 | 11.814 | 26.940 | 1.00 | 33.62 | B | O |
| ATOM | 4650 | N | ASN H | 197 | 12.649 | 11.208 | 25.817 | 1.00 | 34.31 | B | N |
| ATOM | 4651 | CA | ASN H | 197 | 12.370 | 10.074 | 26.691 | 1.00 | 38.83 | B | C |
| ATOM | 4652 | CB | ASN H | 197 | 11.043 | 9.417 | 26.318 | 1.00 | 20.00 | B | C |
| ATOM | 4653 | CG | ASN H | 197 | 9.848 | 10.251 | 26.729 | 1.00 | 20.00 | B | C |
| ATOM | 4654 | OD1 | ASN H | 197 | 9.957 | 11.129 | 27.585 | 1.00 | 20.00 | B | O |
| ATOM | 4655 | ND2 | ASN H | 197 | 8.722 | 10.042 | 26.059 | 1.00 | 20.00 | B | N |
| ATOM | 4656 | C | ASN H | 197 | 13.492 | 9.042 | 26.685 | 1.00 | 39.90 | B | C |
| ATOM | 4657 | O | ASN H | 197 | 14.026 | 8.687 | 27.735 | 1.00 | 39.04 | B | O |
| ATOM | 4658 | N | TYR H | 198 | 13.871 | 8.590 | 25.495 | 1.00 | 35.09 | B | N |
| ATOM | 4659 | CA | TYR H | 198 | 14.992 | 7.672 | 25.355 | 1.00 | 41.03 | B | C |
| ATOM | 4660 | CB | TYR H | 198 | 15.246 | 7.355 | 23.882 | 1.00 | 20.00 | B | C |
| ATOM | 4661 | CG | TYR H | 198 | 14.109 | 6.617 | 23.218 | 1.00 | 20.00 | B | C |
| ATOM | 4662 | CD1 | TYR H | 198 | 14.014 | 5.235 | 23.294 | 1.00 | 20.00 | B | C |
| ATOM | 4663 | CE1 | TYR H | 198 | 12.963 | 4.558 | 22.712 | 1.00 | 20.00 | B | C |
| ATOM | 4664 | CZ | TYR H | 198 | 11.999 | 5.265 | 22.024 | 1.00 | 20.00 | B | C |
| ATOM | 4665 | OH | TYR H | 198 | 10.967 | 4.598 | 21.403 | 1.00 | 20.00 | B | O |
| ATOM | 4666 | CE2 | TYR H | 198 | 12.073 | 6.638 | 21.934 | 1.00 | 20.00 | B | C |
| ATOM | 4667 | CD2 | TYR H | 198 | 13.120 | 7.305 | 22.533 | 1.00 | 20.00 | B | C |
| ATOM | 4668 | C | TYR H | 198 | 16.252 | 8.234 | 26.002 | 1.00 | 43.65 | B | C |
| ATOM | 4669 | O | TYR H | 198 | 16.940 | 7.533 | 26.744 | 1.00 | 52.37 | B | O |
| ATOM | 4670 | N | ILE H | 199 | 16.464 | 9.537 | 25.831 | 1.00 | 35.86 | B | N |
| ATOM | 4671 | CA | ILE H | 199 | 17.634 | 10.219 | 26.386 | 1.00 | 45.39 | B | C |
| ATOM | 4672 | CB | ILE H | 199 | 17.722 | 11.682 | 25.904 | 1.00 | 20.00 | B | C |
| ATOM | 4673 | CG1 | ILE H | 199 | 18.102 | 11.741 | 24.425 | 1.00 | 20.00 | B | C |
| ATOM | 4674 | CD1 | ILE H | 199 | 18.674 | 13.078 | 24.004 | 1.00 | 20.00 | B | C |
| ATOM | 4675 | CG2 | ILE H | 199 | 18.730 | 12.457 | 26.735 | 1.00 | 20.00 | B | C |
| ATOM | 4676 | C | ILE H | 199 | 17.639 | 10.200 | 27.912 | 1.00 | 37.89 | B | C |
| ATOM | 4677 | O | ILE H | 199 | 18.667 | 9.941 | 28.538 | 1.00 | 45.28 | B | O |
| ATOM | 4678 | N | ASP H | 200 | 16.492 | 10.508 | 28.505 | 1.00 | 37.36 | B | N |
| ATOM | 4679 | CA | ASP H | 200 | 16.368 | 10.551 | 29.954 | 1.00 | 46.66 | B | C |
| ATOM | 4680 | CB | ASP H | 200 | 14.992 | 11.080 | 30.354 | 1.00 | 20.00 | B | C |
| ATOM | 4681 | CG | ASP H | 200 | 14.837 | 12.564 | 30.083 | 1.00 | 20.00 | B | C |
| ATOM | 4682 | OD1 | ASP H | 200 | 15.826 | 13.308 | 30.264 | 1.00 | 20.00 | B | O |
| ATOM | 4683 | OD2 | ASP H | 200 | 13.718 | 12.992 | 29.721 | 1.00 | 20.00 | B | O |
| ATOM | 4684 | C | ASP H | 200 | 16.600 | 9.181 | 30.581 | 1.00 | 51.05 | B | C |
| ATOM | 4685 | O | ASP H | 200 | 16.986 | 9.081 | 31.745 | 1.00 | 57.39 | B | O |
| ATOM | 4686 | N | LYS H | 201 | 16.346 | 8.129 | 29.810 | 1.00 | 57.68 | B | N |
| ATOM | 4687 | CA | LYS H | 201 | 16.517 | 6.766 | 30.299 | 1.00 | 44.02 | B | C |
| ATOM | 4688 | CB | LYS H | 201 | 15.747 | 5.778 | 29.423 | 1.00 | 20.00 | B | C |
| ATOM | 4689 | CG | LYS H | 201 | 14.242 | 5.854 | 29.602 | 1.00 | 20.00 | B | C |
| ATOM | 4690 | CD | LYS H | 201 | 13.531 | 4.795 | 28.781 | 1.00 | 20.00 | B | C |
| ATOM | 4691 | CE | LYS H | 201 | 12.030 | 5.042 | 28.737 | 1.00 | 20.00 | B | C |
| ATOM | 4692 | NZ | LYS H | 201 | 11.346 | 4.192 | 27.722 | 1.00 | 20.00 | B | N |
| ATOM | 4693 | C | LYS H | 201 | 17.985 | 6.366 | 30.373 | 1.00 | 43.56 | B | C |
| ATOM | 4694 | O | LYS H | 201 | 18.348 | 5.447 | 31.105 | 1.00 | 51.34 | B | O |
| ATOM | 4695 | N | GLN H | 202 | 18.832 | 7.074 | 29.633 | 1.00 | 46.06 | B | N |
| ATOM | 4696 | CA | GLN H | 202 | 20.216 | 6.648 | 29.445 | 1.00 | 44.95 | B | C |
| ATOM | 4697 | CB | GLN H | 202 | 20.507 | 6.398 | 27.963 | 1.00 | 20.00 | B | C |
| ATOM | 4698 | CG | GLN H | 202 | 19.685 | 5.270 | 27.351 | 1.00 | 20.00 | B | C |
| ATOM | 4699 | CD | GLN H | 202 | 19.894 | 5.134 | 25.853 | 1.00 | 20.00 | B | C |
| ATOM | 4700 | OE1 | GLN H | 202 | 20.323 | 6.075 | 25.184 | 1.00 | 20.00 | B | O |
| ATOM | 4701 | NE2 | GLN H | 202 | 19.513 | 3.984 | 25.309 | 1.00 | 20.00 | B | N |
| ATOM | 4702 | C | GLN H | 202 | 21.245 | 7.616 | 30.044 | 1.00 | 42.35 | B | C |
| ATOM | 4703 | O | GLN H | 202 | 22.376 | 7.224 | 30.335 | 1.00 | 47.00 | B | O |
| ATOM | 4704 | N | LEU H | 203 | 20.852 | 8.874 | 30.228 | 1.00 | 49.55 | B | N |
| ATOM | 4705 | CA | LEU H | 203 | 21.762 | 9.902 | 30.735 | 1.00 | 50.77 | B | C |
| ATOM | 4706 | CB | LEU H | 203 | 21.119 | 11.283 | 30.618 | 1.00 | 20.00 | B | C |
| ATOM | 4707 | CG | LEU H | 203 | 20.836 | 11.775 | 29.202 | 1.00 | 20.00 | B | C |
| ATOM | 4708 | CD1 | LEU H | 203 | 20.093 | 13.100 | 29.256 | 1.00 | 20.00 | B | C |
| ATOM | 4709 | CD2 | LEU H | 203 | 22.134 | 11.910 | 28.422 | 1.00 | 20.00 | B | C |
| ATOM | 4710 | C | LEU H | 203 | 22.148 | 9.656 | 32.190 | 1.00 | 52.51 | B | C |
| ATOM | 4711 | O | LEU H | 203 | 21.273 | 9.564 | 33.050 | 1.00 | 46.02 | B | O |
| ATOM | 4712 | N | LEU H | 204 | 23.439 | 9.786 | 32.490 | 1.00 | 51.45 | B | N |
| ATOM | 4713 | CA | LEU H | 204 | 23.928 | 9.661 | 33.865 | 1.00 | 58.44 | B | C |
| ATOM | 4714 | CB | LEU H | 204 | 25.423 | 9.333 | 33.885 | 1.00 | 20.00 | B | C |
| ATOM | 4715 | CG | LEU H | 204 | 25.848 | 7.919 | 33.484 | 1.00 | 20.00 | B | C |
| ATOM | 4716 | CD1 | LEU H | 204 | 27.346 | 7.764 | 33.658 | 1.00 | 20.00 | B | C |
| ATOM | 4717 | CD2 | LEU H | 204 | 25.112 | 6.874 | 34.306 | 1.00 | 20.00 | B | C |
| ATOM | 4718 | C | LEU H | 204 | 23.661 | 10.930 | 34.667 | 1.00 | 54.99 | B | C |
| ATOM | 4719 | O | LEU H | 204 | 23.740 | 12.035 | 34.131 | 1.00 | 55.13 | B | O |
| ATOM | 4720 | N | PRO H | 205 | 23.414 | 10.775 | 35.977 | 1.00 | 58.64 | B | N |
| ATOM | 4721 | CA | PRO H | 205 | 22.981 | 11.935 | 36.734 | 1.00 | 52.19 | B | C |
| ATOM | 4722 | CB | PRO H | 205 | 22.776 | 11.363 | 38.137 | 1.00 | 20.00 | B | C |
| ATOM | 4723 | CG | PRO H | 205 | 23.735 | 10.190 | 38.219 | 1.00 | 20.00 | B | C |
| ATOM | 4724 | CD | PRO H | 205 | 24.191 | 9.850 | 36.818 | 1.00 | 20.00 | B | C |
| ATOM | 4725 | C | PRO H | 205 | 24.112 | 12.948 | 36.754 | 1.00 | 54.30 | B | C |
| ATOM | 4726 | O | PRO H | 205 | 23.898 | 14.128 | 37.030 | 1.00 | 70.22 | B | O |
| ATOM | 4727 | N | ILE H | 206 | 25.316 | 12.460 | 36.481 | 1.00 | 62.70 | B | N |
| ATOM | 4728 | CA | ILE H | 206 | 26.487 | 13.308 | 36.354 | 1.00 | 68.19 | B | C |
| ATOM | 4729 | CB | ILE H | 206 | 27.225 | 13.445 | 37.696 | 1.00 | 20.00 | B | C |
| ATOM | 4730 | CG1 | ILE H | 206 | 26.350 | 14.186 | 38.709 | 1.00 | 20.00 | B | C |
| ATOM | 4731 | CD1 | ILE H | 206 | 26.843 | 14.078 | 40.136 | 1.00 | 20.00 | B | C |
| ATOM | 4732 | CG2 | ILE H | 206 | 28.553 | 14.161 | 37.505 | 1.00 | 20.00 | B | C |
| ATOM | 4733 | C | ILE H | 206 | 27.439 | 12.723 | 35.321 | 1.00 | 62.35 | B | C |
| ATOM | 4734 | O | ILE H | 206 | 27.594 | 11.505 | 35.221 | 1.00 | 67.16 | B | O |
| ATOM | 4735 | N | VAL H | 207 | 28.023 | 13.596 | 34.510 | 1.00 | 52.22 | B | N |
| ATOM | 4736 | CA | VAL H | 207 | 28.952 | 13.168 | 33.480 | 1.00 | 51.31 | B | C |
| ATOM | 4737 | CB | VAL H | 207 | 28.568 | 13.742 | 32.111 | 1.00 | 20.00 | B | C |
| ATOM | 4738 | CG1 | VAL H | 207 | 29.545 | 13.261 | 31.047 | 1.00 | 20.00 | B | C |
| ATOM | 4739 | CG2 | VAL H | 207 | 27.136 | 13.356 | 31.758 | 1.00 | 20.00 | B | C |
| ATOM | 4740 | C | VAL H | 207 | 30.378 | 13.582 | 33.821 | 1.00 | 62.09 | B | C |
| ATOM | 4741 | O | VAL H | 207 | 30.751 | 14.746 | 33.673 | 1.00 | 58.88 | B | O |
| ATOM | 4742 | N | ASN H | 208 | 31.140 | 12.641 | 34.367 | 1.00 | 71.72 | B | N |
| ATOM | 4743 | CA | ASN H | 208 | 32.533 | 12.886 | 34.707 | 1.00 | 70.92 | B | C |
| ATOM | 4744 | CB | ASN H | 208 | 32.855 | 12.307 | 36.086 | 1.00 | 20.00 | B | C |
| ATOM | 4745 | CG | ASN H | 208 | 32.187 | 13.074 | 37.209 | 1.00 | 20.00 | B | C |
| ATOM | 4746 | OD1 | ASN H | 208 | 31.862 | 14.252 | 37.065 | 1.00 | 20.00 | B | O |
| ATOM | 4747 | ND2 | ASN H | 208 | 31.993 | 12.411 | 38.344 | 1.00 | 20.00 | B | N |
| ATOM | 4748 | C | ASN H | 208 | 33.484 | 12.312 | 33.665 | 1.00 | 63.05 | B | C |
| ATOM | 4749 | O | ASN H | 208 | 33.051 | 11.778 | 32.645 | 1.00 | 61.34 | B | O |
| ATOM | 4750 | N | LYS H | 209 | 34.776 | 12.337 | 33.979 | 1.00 | 68.91 | B | N |
| ATOM | 4751 | CA | LYS H | 209 | 35.811 | 11.927 | 33.037 | 1.00 | 59.11 | B | C |
| ATOM | 4752 | CB | LYS H | 209 | 37.190 | 12.378 | 33.528 | 1.00 | 20.00 | B | C |
| ATOM | 4753 | CG | LYS H | 209 | 37.362 | 13.888 | 33.639 | 1.00 | 20.00 | B | C |
| ATOM | 4754 | CD | LYS H | 209 | 38.689 | 14.243 | 34.302 | 1.00 | 20.00 | B | C |
| ATOM | 4755 | CE | LYS H | 209 | 38.914 | 15.748 | 34.344 | 1.00 | 20.00 | B | C |
| ATOM | 4756 | NZ | LYS H | 209 | 40.286 | 16.091 | 34.815 | 1.00 | 20.00 | B | N |
| ATOM | 4757 | C | LYS H | 209 | 35.810 | 10.415 | 32.817 | 1.00 | 50.69 | B | C |
| ATOM | 4758 | O | LYS H | 209 | 36.500 | 9.910 | 31.932 | 1.00 | 51.01 | B | O |
| ATOM | 4759 | N | GLN H | 210 | 35.116 | 9.691 | 33.689 | 1.00 | 44.68 | B | N |
| ATOM | 4760 | CA | GLN H | 210 | 34.957 | 8.249 | 33.528 | 1.00 | 49.52 | B | C |
| ATOM | 4761 | CB | GLN H | 210 | 34.767 | 7.581 | 34.890 | 1.00 | 20.00 | B | C |
| ATOM | 4762 | CG | GLN H | 210 | 35.976 | 7.671 | 35.803 | 1.00 | 20.00 | B | C |
| ATOM | 4763 | CD | GLN H | 210 | 35.727 | 7.036 | 37.156 | 1.00 | 20.00 | B | C |
| ATOM | 4764 | OE1 | GLN H | 210 | 34.591 | 6.983 | 37.629 | 1.00 | 20.00 | B | O |
| ATOM | 4765 | NE2 | GLN H | 210 | 36.792 | 6.571 | 37.798 | 1.00 | 20.00 | B | N |
| ATOM | 4766 | C | GLN H | 210 | 33.765 | 7.937 | 32.632 | 1.00 | 62.08 | B | C |
| ATOM | 4767 | O | GLN H | 210 | 33.906 | 7.310 | 31.580 | 1.00 | 80.43 | B | O |
| ATOM | 4768 | N | SER H | 211 | 32.599 | 8.424 | 33.040 | 1.00 | 58.92 | B | N |
| ATOM | 4769 | CA | SER H | 211 | 31.384 | 8.297 | 32.251 | 1.00 | 47.88 | B | C |
| ATOM | 4770 | CB | SER H | 211 | 30.270 | 9.156 | 32.857 | 1.00 | 20.00 | B | C |
| ATOM | 4771 | OG | SER H | 211 | 30.733 | 10.462 | 33.169 | 1.00 | 20.00 | B | O |
| ATOM | 4772 | C | SER H | 211 | 31.609 | 8.665 | 30.783 | 1.00 | 57.32 | B | C |
| ATOM | 4773 | O | SER H | 211 | 31.138 | 7.967 | 29.884 | 1.00 | 50.74 | B | O |
| ATOM | 4774 | N | CYS H | 212 | 32.412 | 9.699 | 30.545 | 1.00 | 44.34 | B | N |
| ATOM | 4775 | CA | CYS H | 212 | 32.520 | 10.292 | 29.213 | 1.00 | 57.52 | B | C |
| ATOM | 4776 | CB | CYS H | 212 | 33.762 | 11.204 | 29.075 | 1.00 | 20.00 | B | C |
| ATOM | 4777 | SG | CYS H | 212 | 33.808 | 12.659 | 30.229 | 1.00 | 20.00 | B | S |
| ATOM | 4778 | C | CYS H | 212 | 32.395 | 9.264 | 28.073 | 1.00 | 60.91 | B | C |
| ATOM | 4779 | O | CYS H | 212 | 31.468 | 9.353 | 27.272 | 1.00 | 52.17 | B | O |
| ATOM | 4780 | N | SER H | 213 | 33.381 | 8.374 | 27.965 | 1.00 | 21.55 | B | N |
| ATOM | 4781 | CA | SER H | 213 | 33.462 | 7.484 | 26.814 | 1.00 | 21.67 | B | C |
| ATOM | 4782 | CB | SER H | 213 | 34.765 | 6.678 | 26.855 | 1.00 | 21.81 | B | C |
| ATOM | 4783 | OG | SER H | 213 | 35.892 | 7.530 | 26.960 | 1.00 | 23.00 | B | O |
| ATOM | 4784 | C | SER H | 213 | 32.284 | 6.537 | 26.686 | 1.00 | 21.45 | B | C |
| ATOM | 4785 | O | SER H | 213 | 31.711 | 6.410 | 25.603 | 1.00 | 21.98 | B | O |
| ATOM | 4786 | N | ILE H | 214 | 31.895 | 5.885 | 27.776 | 1.00 | 20.90 | B | N |
| ATOM | 4787 | CA | ILE H | 214 | 30.691 | 5.081 | 27.699 | 1.00 | 20.45 | B | C |
| ATOM | 4788 | CB | ILE H | 214 | 30.475 | 4.234 | 28.960 | 1.00 | 20.53 | B | C |
| ATOM | 4789 | CG1 | ILE H | 214 | 31.411 | 3.025 | 28.946 | 1.00 | 20.46 | B | C |
| ATOM | 4790 | CD1 | ILE H | 214 | 31.049 | 1.951 | 29.952 | 1.00 | 21.75 | B | C |
| ATOM | 4791 | CG2 | ILE H | 214 | 29.021 | 3.787 | 29.052 | 1.00 | 20.34 | B | C |
| ATOM | 4792 | C | ILE H | 214 | 29.526 | 6.035 | 27.523 | 1.00 | 20.15 | B | C |
| ATOM | 4793 | O | ILE H | 214 | 28.694 | 5.870 | 26.631 | 1.00 | 19.91 | B | O |
| ATOM | 4794 | N | SER H | 215 | 29.528 | 7.080 | 28.346 | 1.00 | 19.84 | B | N |
| ATOM | 4795 | CA | SER H | 215 | 28.475 | 8.080 | 28.330 | 1.00 | 19.58 | B | C |
| ATOM | 4796 | CB | SER H | 215 | 28.663 | 9.070 | 29.478 | 1.00 | 19.76 | B | C |
| ATOM | 4797 | OG | SER H | 215 | 27.637 | 10.048 | 29.470 | 1.00 | 19.97 | B | O |
| ATOM | 4798 | C | SER H | 215 | 28.460 | 8.825 | 27.017 | 1.00 | 19.34 | B | C |
| ATOM | 4799 | O | SER H | 215 | 27.406 | 9.053 | 26.446 | 1.00 | 19.54 | B | O |
| ATOM | 4800 | N | ASN H | 216 | 29.638 | 9.187 | 26.527 | 1.00 | 18.80 | B | N |
| ATOM | 4801 | CA | ASN H | 216 | 29.724 | 9.885 | 25.258 | 1.00 | 18.36 | B | C |
| ATOM | 4802 | CB | ASN H | 216 | 31.156 | 10.336 | 24.972 | 1.00 | 18.41 | B | C |
| ATOM | 4803 | CG | ASN H | 216 | 31.647 | 11.377 | 25.959 | 1.00 | 19.55 | B | C |
| ATOM | 4804 | OD1 | ASN H | 216 | 31.028 | 12.428 | 26.133 | 1.00 | 20.52 | B | O |
| ATOM | 4805 | ND2 | ASN H | 216 | 32.779 | 11.100 | 26.597 | 1.00 | 20.43 | B | N |
| ATOM | 4806 | C | ASN H | 216 | 29.211 | 9.010 | 24.129 | 1.00 | 17.78 | B | C |
| ATOM | 4807 | O | ASN H | 216 | 28.481 | 9.479 | 23.255 | 1.00 | 17.91 | B | O |
| ATOM | 4808 | N | ILE H | 217 | 29.570 | 7.730 | 24.150 | 1.00 | 17.02 | B | N |
| ATOM | 4809 | CA | ILE H | 217 | 29.110 | 6.845 | 23.095 | 1.00 | 16.60 | B | C |
| ATOM | 4810 | CB | ILE H | 217 | 29.718 | 5.439 | 23.210 | 1.00 | 16.46 | B | C |
| ATOM | 4811 | CG1 | ILE H | 217 | 31.093 | 5.403 | 22.540 | 1.00 | 16.26 | B | C |
| ATOM | 4812 | CD1 | ILE H | 217 | 31.610 | 4.001 | 22.276 | 1.00 | 16.44 | B | C |
| ATOM | 4813 | CG2 | ILE H | 217 | 28.791 | 4.408 | 22.591 | 1.00 | 16.70 | B | C |
| ATOM | 4814 | C | ILE H | 217 | 27.599 | 6.750 | 23.148 | 1.00 | 16.73 | B | C |
| ATOM | 4815 | O | ILE H | 217 | 26.923 | 6.809 | 22.116 | 1.00 | 16.80 | B | O |
| ATOM | 4816 | N | GLU H | 218 | 27.064 | 6.639 | 24.360 | 1.00 | 17.02 | B | N |
| ATOM | 4817 | CA | GLU H | 218 | 25.631 | 6.500 | 24.526 | 1.00 | 17.70 | B | C |
| ATOM | 4818 | CB | GLU H | 218 | 25.266 | 6.321 | 26.000 | 1.00 | 18.20 | B | C |
| ATOM | 4819 | CG | GLU H | 218 | 25.955 | 5.155 | 26.685 | 1.00 | 21.31 | B | C |
| ATOM | 4820 | CD | GLU H | 218 | 25.270 | 4.774 | 27.986 | 1.00 | 25.72 | B | C |
| ATOM | 4821 | OE1 | GLU H | 218 | 25.558 | 5.409 | 29.023 | 1.00 | 26.96 | B | O |
| ATOM | 4822 | OE2 | GLU H | 218 | 24.447 | 3.835 | 27.975 | 1.00 | 27.07 | B | O |
| ATOM | 4823 | C | GLU H | 218 | 24.951 | 7.742 | 23.995 | 1.00 | 17.29 | B | C |
| ATOM | 4824 | O | GLU H | 218 | 23.942 | 7.660 | 23.312 | 1.00 | 17.62 | B | O |
| ATOM | 4825 | N | THR H | 219 | 25.482 | 8.931 | 24.255 | 1.00 | 16.62 | B | N |
| ATOM | 4826 | CA | THR H | 219 | 24.786 | 10.141 | 23.789 | 1.00 | 15.95 | B | C |
| ATOM | 4827 | CB | THR H | 219 | 25.475 | 11.413 | 24.325 | 1.00 | 15.95 | B | C |
| ATOM | 4828 | OG1 | THR H | 219 | 25.394 | 11.438 | 25.756 | 1.00 | 15.63 | B | O |
| ATOM | 4829 | CG2 | THR H | 219 | 24.814 | 12.661 | 23.759 | 1.00 | 15.97 | B | C |
| ATOM | 4830 | C | THR H | 219 | 24.646 | 10.261 | 22.254 | 1.00 | 15.64 | B | C |
| ATOM | 4831 | O | THR H | 219 | 23.557 | 10.569 | 21.720 | 1.00 | 15.93 | B | O |
| ATOM | 4832 | N | VAL H | 220 | 25.737 | 9.974 | 21.550 | 1.00 | 14.98 | B | N |
| ATOM | 4833 | CA | VAL H | 220 | 25.773 | 10.074 | 20.093 | 1.00 | 14.51 | B | C |
| ATOM | 4834 | CB | VAL H | 220 | 27.177 | 9.738 | 19.559 | 1.00 | 14.29 | B | C |
| ATOM | 4835 | CG1 | VAL H | 220 | 27.191 | 9.771 | 18.041 | 1.00 | 14.24 | B | C |
| ATOM | 4836 | CG2 | VAL H | 220 | 28.213 | 10.693 | 20.137 | 1.00 | 14.10 | B | C |
| ATOM | 4837 | C | VAL H | 220 | 24.779 | 9.088 | 19.498 | 1.00 | 14.64 | B | C |
| ATOM | 4838 | O | VAL H | 220 | 24.030 | 9.390 | 18.544 | 1.00 | 14.79 | B | O |
| ATOM | 4839 | N | ILE H | 221 | 24.762 | 7.904 | 20.097 | 1.00 | 14.43 | B | N |
| ATOM | 4840 | CA | ILE H | 221 | 23.860 | 6.856 | 19.674 | 1.00 | 14.36 | B | C |
| ATOM | 4841 | CB | ILE H | 221 | 24.067 | 5.569 | 20.487 | 1.00 | 14.03 | B | C |
| ATOM | 4842 | CG1 | ILE H | 221 | 25.394 | 4.909 | 20.110 | 1.00 | 13.72 | B | C |
| ATOM | 4843 | CD1 | ILE H | 221 | 25.493 | 4.526 | 18.649 | 1.00 | 14.17 | B | C |
| ATOM | 4844 | CG2 | ILE H | 221 | 22.908 | 4.611 | 20.267 | 1.00 | 13.91 | B | C |
| ATOM | 4845 | C | ILE H | 221 | 22.443 | 7.351 | 19.869 | 1.00 | 14.82 | B | C |
| ATOM | 4846 | O | ILE H | 221 | 21.589 | 7.132 | 19.023 | 1.00 | 14.82 | B | O |
| ATOM | 4847 | N | GLU H | 222 | 22.195 | 8.039 | 20.978 | 1.00 | 15.51 | B | N |
| ATOM | 4848 | CA | GLU H | 222 | 20.857 | 8.551 | 21.246 | 1.00 | 16.52 | B | C |
| ATOM | 4849 | CB | GLU H | 222 | 20.733 | 9.113 | 22.663 | 1.00 | 16.80 | B | C |
| ATOM | 4850 | CG | GLU H | 222 | 20.725 | 8.043 | 23.747 | 1.00 | 19.14 | B | C |
| ATOM | 4851 | CD | GLU H | 222 | 19.637 | 8.267 | 24.780 | 1.00 | 22.62 | B | C |
| ATOM | 4852 | OE1 | GLU H | 222 | 19.965 | 8.662 | 25.919 | 1.00 | 24.35 | B | O |
| ATOM | 4853 | OE2 | GLU H | 222 | 18.453 | 8.039 | 24.454 | 1.00 | 23.98 | B | O |
| ATOM | 4854 | C | GLU H | 222 | 20.400 | 9.566 | 20.199 | 1.00 | 16.65 | B | C |
| ATOM | 4855 | O | GLU H | 222 | 19.255 | 9.541 | 19.792 | 1.00 | 16.95 | B | O |
| ATOM | 4856 | N | PHE H | 223 | 21.292 | 10.444 | 19.752 | 1.00 | 16.70 | B | N |
| ATOM | 4857 | CA | PHE H | 223 | 20.971 | 11.413 | 18.690 | 1.00 | 16.95 | B | C |
| ATOM | 4858 | CB | PHE H | 223 | 22.259 | 12.227 | 18.509 | 1.00 | 16.85 | B | C |
| ATOM | 4859 | CG | PHE H | 223 | 22.154 | 13.406 | 17.593 | 1.00 | 17.23 | B | C |
| ATOM | 4860 | CD1 | PHE H | 223 | 21.522 | 14.570 | 17.995 | 1.00 | 17.98 | B | C |
| ATOM | 4861 | CE1 | PHE H | 223 | 21.467 | 15.667 | 17.155 | 1.00 | 18.28 | B | C |
| ATOM | 4862 | CZ | PHE H | 223 | 22.071 | 15.616 | 15.914 | 1.00 | 18.19 | B | C |
| ATOM | 4863 | CE2 | PHE H | 223 | 22.726 | 14.469 | 15.516 | 1.00 | 18.14 | B | C |
| ATOM | 4864 | CD2 | PHE H | 223 | 22.776 | 13.380 | 16.357 | 1.00 | 17.75 | B | C |
| ATOM | 4865 | C | PHE H | 223 | 20.601 | 10.675 | 17.371 | 1.00 | 17.28 | B | C |
| ATOM | 4866 | O | PHE H | 223 | 19.564 | 10.951 | 16.650 | 1.00 | 17.17 | B | O |
| ATOM | 4867 | N | GLN H | 224 | 21.422 | 9.671 | 17.091 | 1.00 | 17.62 | B | N |
| ATOM | 4868 | CA | GLN H | 224 | 21.209 | 8.880 | 15.894 | 1.00 | 18.16 | B | C |
| ATOM | 4869 | CB | GLN H | 224 | 22.390 | 7.930 | 15.652 | 1.00 | 18.51 | B | C |
| ATOM | 4870 | CG | GLN H | 224 | 23.751 | 8.594 | 15.888 | 1.00 | 20.06 | B | C |
| ATOM | 4871 | CD | GLN H | 224 | 24.867 | 8.009 | 15.039 | 1.00 | 22.03 | B | C |
| ATOM | 4872 | OE1 | GLN H | 224 | 24.920 | 6.800 | 14.806 | 1.00 | 22.15 | B | O |
| ATOM | 4873 | NE2 | GLN H | 224 | 25.783 | 8.864 | 14.595 | 1.00 | 22.11 | B | N |
| ATOM | 4874 | C | GLN H | 224 | 19.855 | 8.158 | 15.956 | 1.00 | 18.04 | B | C |
| ATOM | 4875 | O | GLN H | 224 | 19.196 | 7.981 | 14.932 | 1.00 | 18.17 | B | O |
| ATOM | 4876 | N | GLN H | 225 | 19.488 | 7.701 | 17.156 | 1.00 | 17.90 | B | N |
| ATOM | 4877 | CA | GLN H | 225 | 18.192 | 7.079 | 17.492 | 1.00 | 17.95 | B | C |
| ATOM | 4878 | CB | GLN H | 225 | 18.271 | 6.408 | 18.861 | 1.00 | 18.13 | B | C |
| ATOM | 4879 | CG | GLN H | 225 | 19.251 | 5.257 | 18.893 | 1.00 | 19.04 | B | C |
| ATOM | 4880 | CD | GLN H | 225 | 18.854 | 4.196 | 19.891 | 1.00 | 21.22 | B | C |
| ATOM | 4881 | OE1 | GLN H | 225 | 17.834 | 4.321 | 20.569 | 1.00 | 22.55 | B | O |
| ATOM | 4882 | NE2 | GLN H | 225 | 19.651 | 3.142 | 19.985 | 1.00 | 22.48 | B | N |
| ATOM | 4883 | C | GLN H | 225 | 16.993 | 8.032 | 17.430 | 1.00 | 17.84 | B | C |
| ATOM | 4884 | O | GLN H | 225 | 15.872 | 7.657 | 17.090 | 1.00 | 17.71 | B | O |
| ATOM | 4885 | N | LYS H | 226 | 18.593 | 9.729 | 17.758 | 1.00 | 17.95 | B | N |
| ATOM | 4886 | CA | LYS H | 226 | 17.704 | 10.870 | 17.777 | 1.00 | 18.00 | B | C |
| ATOM | 4887 | CB | LYS H | 226 | 18.471 | 12.167 | 18.045 | 1.00 | 18.21 | B | C |
| ATOM | 4888 | CG | LYS H | 226 | 19.258 | 12.240 | 19.342 | 1.00 | 18.50 | B | C |
| ATOM | 4889 | CD | LYS H | 226 | 18.743 | 13.353 | 20.242 | 1.00 | 19.87 | B | C |
| ATOM | 4890 | CE | LYS H | 226 | 19.762 | 13.696 | 21.316 | 1.00 | 21.36 | B | C |
| ATOM | 4891 | NZ | LYS H | 226 | 19.374 | 14.909 | 22.085 | 1.00 | 21.83 | B | N |
| ATOM | 4892 | C | LYS H | 226 | 17.251 | 10.914 | 16.350 | 1.00 | 17.84 | B | C |
| ATOM | 4893 | O | LYS H | 226 | 16.195 | 11.458 | 16.046 | 1.00 | 17.94 | B | O |
| ATOM | 4894 | N | ASN H | 227 | 18.025 | 10.304 | 15.460 | 1.00 | 17.70 | B | N |
| ATOM | 4895 | CA | ASN H | 227 | 17.467 | 10.197 | 14.086 | 1.00 | 17.66 | B | C |
| ATOM | 4896 | CB | ASN H | 227 | 18.366 | 9.273 | 13.270 | 1.00 | 17.36 | B | C |
| ATOM | 4897 | CG | ASN H | 227 | 19.700 | 9.884 | 12.927 | 1.00 | 17.40 | B | C |
| ATOM | 4898 | OD1 | ASN H | 227 | 20.438 | 9.337 | 12.107 | 1.00 | 17.84 | B | O |
| ATOM | 4899 | ND2 | ASN H | 227 | 20.025 | 11.011 | 13.545 | 1.00 | 17.22 | B | N |
| ATOM | 4900 | C | ASN H | 227 | 16.016 | 9.596 | 13.857 | 1.00 | 17.76 | B | C |
| ATOM | 4901 | O | ASN H | 227 | 15.214 | 10.239 | 13.175 | 1.00 | 17.92 | B | O |
| ATOM | 4902 | N | ASN H | 228 | 15.655 | 8.431 | 14.418 | 1.00 | 17.78 | B | N |
| ATOM | 4903 | CA | ASN H | 228 | 14.290 | 7.922 | 14.435 | 1.00 | 17.59 | B | C |
| ATOM | 4904 | CB | ASN H | 228 | 14.003 | 7.185 | 15.743 | 1.00 | 17.89 | B | C |
| ATOM | 4905 | CG | ASN H | 228 | 14.845 | 5.934 | 15.903 | 1.00 | 18.43 | B | C |
| ATOM | 4906 | OD1 | ASN H | 228 | 15.432 | 5.698 | 16.959 | 1.00 | 18.74 | B | O |
| ATOM | 4907 | ND2 | ASN H | 228 | 14.908 | 5.124 | 14.852 | 1.00 | 18.61 | B | N |
| ATOM | 4908 | C | ASN H | 228 | 13.335 | 9.091 | 14.265 | 1.00 | 17.09 | B | C |
| ATOM | 4909 | O | ASN H | 228 | 12.380 | 9.032 | 13.490 | 1.00 | 17.17 | B | O |
| ATOM | 4910 | N | ARG H | 229 | 13.267 | 9.880 | 15.306 | 1.00 | 16.33 | B | N |
| ATOM | 4911 | CA | ARG H | 229 | 12.252 | 10.885 | 15.298 | 1.00 | 15.76 | B | C |
| ATOM | 4912 | CB | ARG H | 229 | 11.906 | 11.215 | 16.758 | 1.00 | 15.70 | B | C |
| ATOM | 4913 | CG | ARG H | 229 | 11.667 | 9.916 | 17.580 | 1.00 | 14.99 | B | C |
| ATOM | 4914 | CD | ARG H | 229 | 11.240 | 10.128 | 19.039 | 1.00 | 15.33 | B | C |
| ATOM | 4915 | NE | ARG H | 229 | 12.372 | 10.516 | 19.868 | 1.00 | 15.59 | B | N |
| ATOM | 4916 | CZ | ARG H | 229 | 13.209 | 9.663 | 20.448 | 1.00 | 15.85 | B | C |
| ATOM | 4917 | NH1 | ARG H | 229 | 13.045 | 8.350 | 20.313 | 1.00 | 16.20 | B | N |
| ATOM | 4918 | NH2 | ARG H | 229 | 14.216 | 10.134 | 21.168 | 1.00 | 16.63 | B | N |
| ATOM | 4919 | C | ARG H | 229 | 12.960 | 11.937 | 14.490 | 1.00 | 15.62 | B | C |
| ATOM | 4920 | O | ARG H | 229 | 12.436 | 12.586 | 13.561 | 1.00 | 15.73 | B | O |
| ATOM | 4921 | N | LEU H | 230 | 14.249 | 11.935 | 14.783 | 1.00 | 15.46 | B | N |
| ATOM | 4922 | CA | LEU H | 230 | 15.146 | 12.972 | 14.402 | 1.00 | 15.22 | B | C |
| ATOM | 4923 | CB | LEU H | 230 | 16.334 | 12.863 | 15.348 | 1.00 | 15.08 | B | C |
| ATOM | 4924 | CG | LEU H | 230 | 17.100 | 14.054 | 15.896 | 1.00 | 14.70 | B | C |
| ATOM | 4925 | CD1 | LEU H | 230 | 16.465 | 14.590 | 17.171 | 1.00 | 15.19 | B | C |
| ATOM | 4926 | CD2 | LEU H | 230 | 18.500 | 13.564 | 16.165 | 1.00 | 15.46 | B | C |
| ATOM | 4927 | C | LEU H | 230 | 15.658 | 12.853 | 12.989 | 1.00 | 15.31 | B | C |
| ATOM | 4928 | O | LEU H | 230 | 15.224 | 13.608 | 12.133 | 1.00 | 15.46 | B | O |
| ATOM | 4929 | N | LEU H | 231 | 16.533 | 11.890 | 12.727 | 1.00 | 15.48 | B | N |
| ATOM | 4930 | CA | LEU H | 231 | 17.158 | 11.825 | 11.423 | 1.00 | 15.75 | B | C |
| ATOM | 4931 | CB | LEU H | 231 | 18.215 | 10.722 | 11.423 | 1.00 | 15.90 | B | C |
| ATOM | 4932 | CG | LEU H | 231 | 19.366 | 10.875 | 12.416 | 1.00 | 16.63 | B | C |
| ATOM | 4933 | CD1 | LEU H | 231 | 20.443 | 9.836 | 12.133 | 1.00 | 18.45 | B | C |
| ATOM | 4934 | CD2 | LEU H | 231 | 19.953 | 12.276 | 12.366 | 1.00 | 16.67 | B | C |
| ATOM | 4935 | C | LEU H | 231 | 16.139 | 11.575 | 10.324 | 1.00 | 15.76 | B | C |
| ATOM | 4936 | O | LEU H | 231 | 16.121 | 12.269 | 9.309 | 1.00 | 15.84 | B | O |
| ATOM | 4937 | N | GLU H | 232 | 15.249 | 10.617 | 10.561 | 1.00 | 15.71 | B | N |
| ATOM | 4938 | CA | GLU H | 232 | 14.200 | 10.308 | 9.607 | 1.00 | 15.93 | B | C |
| ATOM | 4939 | CB | GLU H | 232 | 13.470 | 9.012 | 10.001 | 1.00 | 16.15 | B | C |
| ATOM | 4940 | CG | GLU H | 232 | 14.389 | 7.801 | 10.158 | 1.00 | 18.59 | B | C |
| ATOM | 4941 | CD | GLU H | 232 | 14.567 | 6.988 | 8.876 | 1.00 | 21.47 | B | C |
| ATOM | 4942 | OE1 | GLU H | 232 | 13.976 | 7.355 | 7.839 | 1.00 | 22.14 | B | O |
| ATOM | 4943 | OE2 | GLU H | 232 | 15.298 | 5.973 | 8.907 | 1.00 | 23.34 | B | O |
| ATOM | 4944 | C | GLU H | 232 | 13.241 | 11.482 | 9.456 | 1.00 | 15.45 | B | C |
| ATOM | 4945 | O | GLU H | 232 | 12.881 | 11.860 | 8.328 | 1.00 | 15.95 | B | O |
| ATOM | 4946 | N | ILE H | 233 | 12.855 | 12.113 | 10.564 | 1.00 | 14.54 | B | N |
| ATOM | 4947 | CA | ILE H | 233 | 11.941 | 13.221 | 10.375 | 1.00 | 13.69 | B | C |
| ATOM | 4948 | CB | ILE H | 233 | 11.444 | 13.775 | 11.699 | 1.00 | 13.63 | B | C |
| ATOM | 4949 | CG1 | ILE H | 233 | 10.497 | 12.763 | 12.326 | 1.00 | 13.58 | B | C |
| ATOM | 4950 | CD1 | ILE H | 233 | 9.817 | 13.280 | 13.532 | 1.00 | 13.55 | B | C |
| ATOM | 4951 | CG2 | ILE H | 233 | 10.682 | 15.070 | 11.490 | 1.00 | 13.88 | B | C |
| ATOM | 4952 | C | ILE H | 233 | 12.605 | 14.309 | 9.526 | 1.00 | 13.28 | B | C |
| ATOM | 4953 | O | ILE H | 233 | 11.972 | 14.914 | 8.653 | 1.00 | 13.04 | B | O |
| ATOM | 4954 | N | THR H | 234 | 13.888 | 14.544 | 9.773 | 1.00 | 13.18 | B | N |
| ATOM | 4955 | CA | THR H | 234 | 14.620 | 15.575 | 9.063 | 1.00 | 13.39 | B | C |
| ATOM | 4956 | CB | THR H | 234 | 16.082 | 15.655 | 9.574 | 1.00 | 13.21 | B | C |
| ATOM | 4957 | OG1 | THR H | 234 | 16.099 | 15.927 | 10.978 | 1.00 | 13.15 | B | O |
| ATOM | 4958 | CG2 | THR H | 234 | 16.870 | 16.740 | 8.846 | 1.00 | 13.22 | B | C |
| ATOM | 4959 | C | THR H | 234 | 14.686 | 15.273 | 7.577 | 1.00 | 13.76 | B | C |
| ATOM | 4960 | O | THR H | 234 | 14.434 | 16.152 | 6.738 | 1.00 | 13.95 | B | O |
| ATOM | 4961 | N | ARG H | 235 | 14.994 | 14.024 | 7.240 | 1.00 | 14.26 | B | N |
| ATOM | 4962 | CA | ARG H | 235 | 15.119 | 13.686 | 5.831 | 1.00 | 14.92 | B | C |
| ATOM | 4963 | CB | ARG H | 235 | 15.574 | 12.242 | 5.661 | 1.00 | 15.30 | B | C |
| ATOM | 4964 | CG | ARG H | 235 | 14.532 | 11.325 | 5.054 | 1.00 | 16.27 | B | C |
| ATOM | 4965 | CD | ARG H | 235 | 14.829 | 9.882 | 5.408 | 1.00 | 18.52 | B | C |
| ATOM | 4966 | NE | ARG H | 235 | 16.234 | 9.676 | 5.746 | 1.00 | 17.86 | B | N |
| ATOM | 4967 | CZ | ARG H | 235 | 17.190 | 9.425 | 4.858 | 1.00 | 16.91 | B | C |
| ATOM | 4968 | NH1 | ARG H | 235 | 16.898 | 9.354 | 3.566 | 1.00 | 16.59 | B | N |
| ATOM | 4969 | NH2 | ARG H | 235 | 18.438 | 9.249 | 5.267 | 1.00 | 16.86 | B | N |
| ATOM | 4970 | C | ARG H | 235 | 13.766 | 13.887 | 5.176 | 1.00 | 15.05 | B | C |
| ATOM | 4971 | O | ARG H | 235 | 13.663 | 14.432 | 4.073 | 1.00 | 15.15 | B | O |
| ATOM | 4972 | N | GLU H | 236 | 12.727 | 13.474 | 5.894 | 1.00 | 15.18 | B | N |
| ATOM | 4973 | CA | GLU H | 236 | 11.375 | 13.527 | 5.376 | 1.00 | 15.24 | B | C |
| ATOM | 4974 | CB | GLU H | 236 | 10.415 | 12.920 | 6.403 | 1.00 | 15.69 | B | C |
| ATOM | 4975 | CG | GLU H | 236 | 9.176 | 12.265 | 5.822 | 1.00 | 18.22 | B | C |
| ATOM | 4976 | CD | GLU H | 236 | 8.355 | 11.543 | 6.875 | 1.00 | 21.59 | B | C |
| ATOM | 4977 | OE1 | GLU H | 236 | 7.200 | 11.178 | 6.581 | 1.00 | 24.41 | B | O |
| ATOM | 4978 | OE2 | GLU H | 236 | 8.857 | 11.345 | 8.000 | 1.00 | 21.59 | B | O |
| ATOM | 4979 | C | GLU H | 236 | 10.987 | 14.971 | 5.073 | 1.00 | 14.61 | B | C |
| ATOM | 4980 | O | GLU H | 236 | 10.396 | 15.253 | 4.031 | 1.00 | 14.82 | B | O |
| ATOM | 4981 | N | PHE H | 237 | 11.333 | 15.877 | 5.985 | 1.00 | 14.05 | B | N |
| ATOM | 4982 | CA | PHE H | 237 | 11.053 | 17.296 | 5.809 | 1.00 | 13.84 | B | C |
| ATOM | 4983 | CB | PHE H | 237 | 11.316 | 18.065 | 7.105 | 1.00 | 14.04 | B | C |
| ATOM | 4984 | CG | PHE H | 237 | 10.142 | 18.087 | 8.038 | 1.00 | 14.84 | B | C |
| ATOM | 4985 | CD1 | PHE H | 237 | 10.224 | 17.516 | 9.297 | 1.00 | 15.30 | B | C |
| ATOM | 4986 | CE1 | PHE H | 237 | 9.142 | 17.542 | 10.153 | 1.00 | 15.15 | B | C |
| ATOM | 4987 | CZ | PHE H | 237 | 7.961 | 18.135 | 9.755 | 1.00 | 15.19 | B | C |
| ATOM | 4988 | CE2 | PHE H | 237 | 7.864 | 18.704 | 8.502 | 1.00 | 15.02 | B | C |
| ATOM | 4989 | CD2 | PHE H | 237 | 8.949 | 18.677 | 7.651 | 1.00 | 15.03 | B | C |
| ATOM | 4990 | C | PHE H | 237 | 11.830 | 17.909 | 4.654 | 1.00 | 13.51 | B | C |
| ATOM | 4991 | O | PHE H | 237 | 11.293 | 18.687 | 3.866 | 1.00 | 13.43 | B | O |
| ATOM | 4992 | N | SER H | 238 | 13.100 | 17.534 | 4.554 | 1.00 | 13.11 | B | N |
| ATOM | 4993 | CA | SER H | 238 | 13.972 | 18.065 | 3.516 | 1.00 | 12.82 | B | C |
| ATOM | 4994 | CB | SER H | 238 | 15.404 | 17.546 | 3.691 | 1.00 | 12.86 | B | C |
| ATOM | 4995 | OG | SER H | 238 | 15.976 | 17.981 | 4.916 | 1.00 | 12.76 | B | O |
| ATOM | 4996 | C | SER H | 238 | 13.411 | 17.654 | 2.164 | 1.00 | 12.69 | B | C |
| ATOM | 4997 | O | SER H | 238 | 13.437 | 18.425 | 1.202 | 1.00 | 12.63 | B | O |
| ATOM | 4998 | N | VAL H | 239 | 12.910 | 16.426 | 2.107 | 1.00 | 12.59 | B | N |
| ATOM | 4999 | CA | VAL H | 239 | 12.361 | 15.865 | 0.883 | 1.00 | 12.80 | B | C |
| ATOM | 5000 | CB | VAL H | 239 | 12.065 | 14.358 | 1.041 | 1.00 | 12.71 | B | C |
| ATOM | 5001 | CG1 | VAL H | 239 | 11.359 | 13.819 | −0.193 | 1.00 | 13.63 | B | C |
| ATOM | 5002 | CG2 | VAL H | 239 | 13.349 | 13.588 | 1.316 | 1.00 | 13.57 | B | C |
| ATOM | 5003 | C | VAL H | 239 | 11.094 | 16.596 | 0.443 | 1.00 | 12.94 | B | C |
| ATOM | 5004 | O | VAL H | 239 | 10.722 | 16.546 | −0.728 | 1.00 | 13.10 | B | O |
| ATOM | 5005 | N | ASN H | 240 | 10.430 | 17.274 | 1.372 | 1.00 | 4.15 | B | N |
| ATOM | 5006 | CA | ASN H | 240 | 9.074 | 17.741 | 1.109 | 1.00 | 4.33 | B | C |
| ATOM | 5007 | CB | ASN H | 240 | 8.110 | 17.268 | 2.199 | 1.00 | 4.59 | B | C |
| ATOM | 5008 | CG | ASN H | 240 | 7.810 | 15.784 | 2.106 | 1.00 | 5.36 | B | C |
| ATOM | 5009 | OD1 | ASN H | 240 | 7.128 | 15.331 | 1.188 | 1.00 | 6.27 | B | O |
| ATOM | 5010 | ND2 | ASN H | 240 | 8.343 | 15.016 | 3.047 | 1.00 | 7.15 | B | N |
| ATOM | 5011 | C | ASN H | 240 | 8.924 | 19.242 | 0.853 | 1.00 | 4.19 | B | C |
| ATOM | 5012 | O | ASN H | 240 | 7.843 | 19.707 | 0.490 | 1.00 | 4.19 | B | O |
| ATOM | 5013 | N | ALA H | 241 | 10.014 | 19.990 | 0.995 | 1.00 | 3.93 | B | N |
| ATOM | 5014 | CA | ALA H | 241 | 9.948 | 21.447 | 0.930 | 1.00 | 3.90 | B | C |
| ATOM | 5015 | CB | ALA H | 241 | 9.784 | 21.908 | −0.511 | 1.00 | 3.86 | B | C |
| ATOM | 5016 | C | ALA H | 241 | 8.812 | 21.985 | 1.798 | 1.00 | 4.22 | B | C |
| ATOM | 5017 | O | ALA H | 241 | 7.932 | 22.697 | 1.314 | 1.00 | 4.47 | B | O |
| ATOM | 5018 | N | GLY H | 242 | 8.772 | 21.540 | 3.049 | 1.00 | 4.53 | B | N |
| ATOM | 5019 | CA | GLY H | 242 | 8.055 | 22.258 | 4.097 | 1.00 | 4.65 | B | C |
| ATOM | 5020 | C | GLY H | 242 | 6.563 | 21.980 | 4.122 | 1.00 | 4.67 | B | C |
| ATOM | 5021 | O | GLY H | 242 | 5.792 | 22.759 | 4.683 | 1.00 | 5.27 | B | O |
| ATOM | 5022 | N | VAL H | 243 | 6.159 | 20.841 | 3.570 | 1.00 | 4.37 | B | N |
| ATOM | 5023 | CA | VAL H | 243 | 4.755 | 20.449 | 3.577 | 1.00 | 4.32 | B | C |
| ATOM | 5024 | CB | VAL H | 243 | 3.961 | 21.182 | 2.477 | 1.00 | 3.99 | B | C |
| ATOM | 5025 | CG1 | VAL H | 243 | 2.519 | 21.398 | 2.915 | 1.00 | 4.22 | B | C |
| ATOM | 5026 | CG2 | VAL H | 243 | 4.629 | 22.508 | 2.131 | 1.00 | 3.85 | B | C |
| ATOM | 5027 | C | VAL H | 243 | 4.595 | 18.943 | 3.393 | 1.00 | 4.61 | B | C |
| ATOM | 5028 | O | VAL H | 243 | 5.201 | 18.350 | 2.501 | 1.00 | 4.89 | B | O |
| ATOM | 5029 | N | THR H | 244 | 3.764 | 18.333 | 4.231 | 1.00 | 4.95 | B | N |
| ATOM | 5030 | CA | THR H | 244 | 3.600 | 16.886 | 4.220 | 1.00 | 5.71 | B | C |
| ATOM | 5031 | CB | THR H | 244 | 4.318 | 16.226 | 5.410 | 1.00 | 6.11 | B | C |
| ATOM | 5032 | OG1 | THR H | 244 | 5.051 | 17.217 | 6.140 | 1.00 | 7.17 | B | O |
| ATOM | 5033 | CG2 | THR H | 244 | 5.273 | 15.147 | 4.925 | 1.00 | 7.55 | B | C |
| ATOM | 5034 | C | THR H | 244 | 2.129 | 16.505 | 4.268 | 1.00 | 5.61 | B | C |
| ATOM | 5035 | O | THR H | 244 | 1.359 | 17.073 | 5.042 | 1.00 | 5.85 | B | O |
| ATOM | 5036 | N | THR H | 245 | 1.786 | 15.428 | 3.575 | 1.00 | 5.53 | B | N |
| ATOM | 5037 | CA | THR H | 245 | 0.617 | 14.647 | 3.939 | 1.00 | 5.81 | B | C |
| ATOM | 5038 | CB | THR H | 245 | −0.404 | 14.592 | 2.788 | 1.00 | 6.13 | B | C |
| ATOM | 5039 | OG1 | THR H | 245 | −0.888 | 15.915 | 2.517 | 1.00 | 8.11 | B | O |
| ATOM | 5040 | CG2 | THR H | 245 | −1.577 | 13.695 | 3.154 | 1.00 | 6.63 | B | C |
| ATOM | 5041 | C | THR H | 245 | 1.032 | 13.245 | 4.370 | 1.00 | 5.73 | B | C |
| ATOM | 5042 | O | THR H | 245 | 2.189 | 12.861 | 4.202 | 1.00 | 5.86 | B | O |
| ATOM | 5043 | N | PRO H | 246 | 0.060 | 12.433 | 4.804 | 1.00 | 5.68 | B | N |
| ATOM | 5044 | CA | PRO H | 246 | 0.078 | 11.993 | 6.186 | 1.00 | 5.11 | B | C |
| ATOM | 5045 | CB | PRO H | 246 | 0.753 | 10.626 | 6.084 | 1.00 | 5.19 | B | C |
| ATOM | 5046 | CG | PRO H | 246 | 0.360 | 10.124 | 4.675 | 1.00 | 5.90 | B | C |
| ATOM | 5047 | CD | PRO H | 246 | −0.256 | 11.297 | 3.926 | 1.00 | 6.36 | B | C |
| ATOM | 5048 | C | PRO H | 246 | 0.830 | 12.916 | 7.140 | 1.00 | 4.85 | B | C |
| ATOM | 5049 | O | PRO H | 246 | 1.927 | 13.379 | 6.825 | 1.00 | 5.25 | B | O |
| ATOM | 5050 | N | VAL H | 247 | 0.144 | 13.323 | 8.205 | 1.00 | 4.35 | B | N |
| ATOM | 5051 | CA | VAL H | 247 | 0.792 | 13.950 | 9.347 | 1.00 | 3.82 | B | C |
| ATOM | 5052 | CB | VAL H | 247 | −0.198 | 14.817 | 10.157 | 1.00 | 3.52 | B | C |
| ATOM | 5053 | CG1 | VAL H | 247 | 0.558 | 15.754 | 11.087 | 1.00 | 3.62 | B | C |
| ATOM | 5054 | CG2 | VAL H | 247 | −1.091 | 15.615 | 9.217 | 1.00 | 3.60 | B | C |
| ATOM | 5055 | C | VAL H | 247 | 1.447 | 12.899 | 10.242 | 1.00 | 3.69 | B | C |
| ATOM | 5056 | O | VAL H | 247 | 0.761 | 12.124 | 10.913 | 1.00 | 3.36 | B | O |
| ATOM | 5057 | N | SER H | 248 | 2.748 | 12.721 | 10.040 | 1.00 | 3.72 | B | N |
| ATOM | 5058 | CA | SER H | 248 | 3.496 | 11.654 | 10.686 | 1.00 | 3.96 | B | C |
| ATOM | 5059 | CB | SER H | 248 | 4.995 | 11.840 | 10.451 | 1.00 | 4.07 | B | C |
| ATOM | 5060 | OG | SER H | 248 | 5.525 | 12.834 | 11.310 | 1.00 | 4.13 | B | O |
| ATOM | 5061 | C | SER H | 248 | 3.211 | 11.615 | 12.180 | 1.00 | 4.35 | B | C |
| ATOM | 5062 | O | SER H | 248 | 2.890 | 12.635 | 12.788 | 1.00 | 4.36 | B | O |
| ATOM | 5063 | N | THR H | 249 | 3.458 | 10.460 | 12.784 | 1.00 | 4.53 | B | N |
| ATOM | 5064 | CA | THR H | 249 | 3.489 | 10.350 | 14.231 | 1.00 | 4.61 | B | C |
| ATOM | 5065 | CB | THR H | 249 | 3.682 | 8.893 | 14.677 | 1.00 | 4.70 | B | C |
| ATOM | 5066 | OG1 | THR H | 249 | 5.018 | 8.473 | 14.374 | 1.00 | 5.42 | B | O |
| ATOM | 5067 | CG2 | THR H | 249 | 2.695 | 7.984 | 13.961 | 1.00 | 4.88 | B | C |
| ATOM | 5068 | C | THR H | 249 | 4.589 | 11.215 | 14.839 | 1.00 | 4.47 | B | C |
| ATOM | 5069 | O | THR H | 249 | 4.570 | 11.491 | 16.039 | 1.00 | 4.74 | B | O |
| ATOM | 5070 | N | TYR H | 250 | 5.518 | 11.684 | 14.009 | 1.00 | 4.32 | B | N |
| ATOM | 5071 | CA | TYR H | 250 | 6.544 | 12.619 | 14.475 | 1.00 | 4.42 | B | C |
| ATOM | 5072 | CB | TYR H | 250 | 7.811 | 12.531 | 13.625 | 1.00 | 4.91 | B | C |
| ATOM | 5073 | CG | TYR H | 250 | 8.409 | 11.148 | 13.525 | 1.00 | 6.81 | B | C |
| ATOM | 5074 | CD1 | TYR H | 250 | 8.844 | 10.657 | 12.302 | 1.00 | 8.89 | B | C |
| ATOM | 5075 | CE1 | TYR H | 250 | 9.480 | 9.437 | 12.205 | 1.00 | 10.73 | B | C |
| ATOM | 5076 | CZ | TYR H | 250 | 9.685 | 8.685 | 13.341 | 1.00 | 12.28 | B | C |
| ATOM | 5077 | OH | TYR H | 250 | 10.305 | 7.460 | 13.251 | 1.00 | 12.24 | B | O |
| ATOM | 5078 | CE2 | TYR H | 250 | 9.284 | 9.161 | 14.571 | 1.00 | 12.15 | B | C |
| ATOM | 5079 | CD2 | TYR H | 250 | 8.689 | 10.405 | 14.662 | 1.00 | 9.81 | B | C |
| ATOM | 5080 | C | TYR H | 250 | 6.053 | 14.064 | 14.516 | 1.00 | 4.21 | B | C |
| ATOM | 5081 | O | TYR H | 250 | 6.596 | 14.883 | 15.259 | 1.00 | 4.20 | B | O |
| ATOM | 5082 | N | MET H | 251 | 5.148 | 14.413 | 13.605 | 1.00 | 4.14 | B | N |
| ATOM | 5083 | CA | MET H | 251 | 4.517 | 15.732 | 13.632 | 1.00 | 4.15 | B | C |
| ATOM | 5084 | CB | MET H | 251 | 3.798 | 16.014 | 12.313 | 1.00 | 4.14 | B | C |
| ATOM | 5085 | CG | MET H | 251 | 4.720 | 16.097 | 11.115 | 1.00 | 4.51 | B | C |
| ATOM | 5086 | SD | MET H | 251 | 5.733 | 17.587 | 11.115 | 1.00 | 3.81 | B | S |
| ATOM | 5087 | CE | MET H | 251 | 5.601 | 18.077 | 9.395 | 1.00 | 4.55 | B | C |
| ATOM | 5088 | C | MET H | 251 | 3.519 | 15.806 | 14.775 | 1.00 | 4.13 | B | C |
| ATOM | 5089 | O | MET H | 251 | 3.510 | 16.759 | 15.555 | 1.00 | 4.24 | B | O |
| ATOM | 5090 | N | LEU H | 252 | 2.692 | 14.773 | 14.874 | 1.00 | 4.23 | B | N |
| ATOM | 5091 | CA | LEU H | 252 | 1.708 | 14.681 | 15.934 | 1.00 | 4.22 | B | C |
| ATOM | 5092 | CB | LEU H | 252 | 0.302 | 14.881 | 15.369 | 1.00 | 4.14 | B | C |
| ATOM | 5093 | CG | LEU H | 252 | −0.295 | 16.282 | 15.515 | 1.00 | 4.05 | B | C |
| ATOM | 5094 | CD1 | LEU H | 252 | −1.813 | 16.216 | 15.561 | 1.00 | 5.22 | B | C |
| ATOM | 5095 | CD2 | LEU H | 252 | 0.245 | 16.972 | 16.756 | 1.00 | 6.20 | B | C |
| ATOM | 5096 | C | LEU H | 252 | 1.818 | 13.314 | 16.589 | 1.00 | 4.62 | B | C |
| ATOM | 5097 | O | LEU H | 252 | 1.906 | 12.295 | 15.903 | 1.00 | 4.97 | B | O |
| ATOM | 5098 | N | THR H | 253 | 1.986 | 13.314 | 17.906 | 1.00 | 4.90 | B | N |
| ATOM | 5099 | CA | THR H | 253 | 1.718 | 12.126 | 18.704 | 1.00 | 5.57 | B | C |
| ATOM | 5100 | CB | THR H | 253 | 2.147 | 12.323 | 20.168 | 1.00 | 5.55 | B | C |
| ATOM | 5101 | OG1 | THR H | 253 | 3.369 | 13.068 | 20.214 | 1.00 | 6.43 | B | O |
| ATOM | 5102 | CG2 | THR H | 253 | 2.362 | 10.978 | 20.842 | 1.00 | 6.28 | B | C |
| ATOM | 5103 | C | THR H | 253 | 0.235 | 11.775 | 18.673 | 1.00 | 6.03 | B | C |
| ATOM | 5104 | O | THR H | 253 | −0.621 | 12.663 | 18.679 | 1.00 | 6.68 | B | O |
| ATOM | 5105 | N | ASN H | 254 | −0.066 | 10.483 | 18.746 | 1.00 | 6.39 | B | N |
| ATOM | 5106 | CA | ASN H | 254 | −1.396 | 10.040 | 19.141 | 1.00 | 6.79 | B | C |
| ATOM | 5107 | CB | ASN H | 254 | −1.375 | 8.564 | 19.534 | 1.00 | 6.85 | B | C |
| ATOM | 5108 | CG | ASN H | 254 | −2.758 | 7.954 | 19.571 | 1.00 | 6.81 | B | C |
| ATOM | 5109 | OD1 | ASN H | 254 | −3.395 | 7.771 | 18.534 | 1.00 | 7.67 | B | O |
| ATOM | 5110 | ND2 | ASN H | 254 | −3.252 | 7.682 | 20.773 | 1.00 | 6.19 | B | N |
| ATOM | 5111 | C | ASN H | 254 | −1.956 | 10.882 | 20.284 | 1.00 | 7.08 | B | C |
| ATOM | 5112 | O | ASN H | 254 | −2.968 | 11.565 | 20.125 | 1.00 | 6.90 | B | O |
| ATOM | 5113 | N | SER H | 255 | −1.282 | 10.843 | 21.430 | 1.00 | 7.21 | B | N |
| ATOM | 5114 | CA | SER H | 255 | −1.689 | 11.633 | 22.588 | 1.00 | 7.37 | B | C |
| ATOM | 5115 | CB | SER H | 255 | −0.561 | 11.714 | 23.619 | 1.00 | 8.09 | B | C |
| ATOM | 5116 | OG | SER H | 255 | 0.228 | 10.539 | 23.617 | 1.00 | 10.47 | B | O |
| ATOM | 5117 | C | SER H | 255 | −2.102 | 13.036 | 22.163 | 1.00 | 6.82 | B | C |
| ATOM | 5118 | O | SER H | 255 | −3.192 | 13.499 | 22.496 | 1.00 | 6.99 | B | O |
| ATOM | 5119 | N | GLU H | 256 | −1.229 | 13.704 | 21.415 | 1.00 | 6.17 | B | N |
| ATOM | 5120 | CA | GLU H | 256 | −1.485 | 15.072 | 20.981 | 1.00 | 6.13 | B | C |
| ATOM | 5121 | CB | GLU H | 256 | −0.274 | 15.644 | 20.243 | 1.00 | 6.22 | B | C |
| ATOM | 5122 | CG | GLU H | 256 | 0.847 | 16.110 | 21.152 | 1.00 | 8.41 | B | C |
| ATOM | 5123 | CD | GLU H | 256 | 2.209 | 15.761 | 20.596 | 1.00 | 10.26 | B | C |
| ATOM | 5124 | OE1 | GLU H | 256 | 2.292 | 15.472 | 19.384 | 1.00 | 11.90 | B | O |
| ATOM | 5125 | OE2 | GLU H | 256 | 3.162 | 15.626 | 21.392 | 1.00 | 9.55 | B | O |
| ATOM | 5126 | C | GLU H | 256 | −2.706 | 15.126 | 20.079 | 1.00 | 5.76 | B | C |
| ATOM | 5127 | O | GLU H | 256 | −3.684 | 15.810 | 20.380 | 1.00 | 5.93 | B | O |
| ATOM | 5128 | N | LEU H | 257 | −2.633 | 14.420 | 18.956 | 1.00 | 5.67 | B | N |
| ATOM | 5129 | CA | LEU H | 257 | −3.729 | 14.411 | 18.002 | 1.00 | 5.37 | B | C |
| ATOM | 5130 | CB | LEU H | 257 | −3.543 | 13.299 | 16.970 | 1.00 | 5.03 | B | C |
| ATOM | 5131 | CG | LEU H | 257 | −4.781 | 13.003 | 16.121 | 1.00 | 3.65 | B | C |
| ATOM | 5132 | CD1 | LEU H | 257 | −5.114 | 14.184 | 15.222 | 1.00 | 2.00 | B | C |
| ATOM | 5133 | CD2 | LEU H | 257 | −4.592 | 11.737 | 15.305 | 1.00 | 2.14 | B | C |
| ATOM | 5134 | C | LEU H | 257 | −5.050 | 14.223 | 18.732 | 1.00 | 5.64 | B | C |
| ATOM | 5135 | O | LEU H | 257 | −6.020 | 14.934 | 18.475 | 1.00 | 5.81 | B | O |
| ATOM | 5136 | N | LEU H | 258 | −5.061 | 13.300 | 19.687 | 1.00 | 5.75 | B | N |
| ATOM | 5137 | CA | LEU H | 258 | −6.297 | 12.905 | 20.344 | 1.00 | 5.91 | B | C |
| ATOM | 5138 | CB | LEU H | 258 | −6.063 | 11.693 | 21.242 | 1.00 | 6.10 | B | C |
| ATOM | 5139 | CG | LEU H | 258 | −6.438 | 10.358 | 20.605 | 1.00 | 5.75 | B | C |
| ATOM | 5140 | CD1 | LEU H | 258 | −5.970 | 9.206 | 21.477 | 1.00 | 6.33 | B | C |
| ATOM | 5141 | CD2 | LEU H | 258 | −7.939 | 10.287 | 20.372 | 1.00 | 7.33 | B | C |
| ATOM | 5142 | C | LEU H | 258 | −6.908 | 14.049 | 21.142 | 1.00 | 6.03 | B | C |
| ATOM | 5143 | O | LEU H | 258 | −8.113 | 14.290 | 21.071 | 1.00 | 5.73 | B | O |
| ATOM | 5144 | N | SER H | 259 | −6.076 | 14.745 | 21.910 | 1.00 | 6.47 | B | N |
| ATOM | 5145 | CA | SER H | 259 | −6.537 | 15.883 | 22.695 | 1.00 | 6.95 | B | C |
| ATOM | 5146 | CB | SER H | 259 | −5.521 | 16.237 | 23.784 | 1.00 | 7.05 | B | C |
| ATOM | 5147 | OG | SER H | 259 | −4.695 | 17.317 | 23.383 | 1.00 | 6.86 | B | O |
| ATOM | 5148 | C | SER H | 259 | −6.782 | 17.086 | 21.795 | 1.00 | 6.74 | B | C |
| ATOM | 5149 | O | SER H | 259 | −7.592 | 17.959 | 22.110 | 1.00 | 6.86 | B | O |
| ATOM | 5150 | N | LEU H | 260 | −6.074 | 17.125 | 20.670 | 1.00 | 6.31 | B | N |
| ATOM | 5151 | CA | LEU H | 260 | −6.295 | 18.149 | 19.657 | 1.00 | 6.09 | B | C |
| ATOM | 5152 | CB | LEU H | 260 | −5.239 | 18.052 | 18.552 | 1.00 | 6.09 | B | C |
| ATOM | 5153 | CG | LEU H | 260 | −4.235 | 19.203 | 18.446 | 1.00 | 6.24 | B | C |
| ATOM | 5154 | CD1 | LEU H | 260 | −3.350 | 19.030 | 17.222 | 1.00 | 6.53 | B | C |
| ATOM | 5155 | CD2 | LEU H | 260 | −4.944 | 20.549 | 18.407 | 1.00 | 5.98 | B | C |
| ATOM | 5156 | C | LEU H | 260 | −7.692 | 18.026 | 19.056 | 1.00 | 6.25 | B | C |
| ATOM | 5157 | O | LEU H | 260 | −8.428 | 19.010 | 18.963 | 1.00 | 6.37 | B | O |
| ATOM | 5158 | N | ILE H | 261 | −8.024 | 16.825 | 18.596 | 1.00 | 6.63 | B | N |
| ATOM | 5159 | CA | ILE H | 261 | −9.395 | 16.490 | 18.245 | 1.00 | 7.19 | B | C |
| ATOM | 5160 | CB | ILE H | 261 | −9.568 | 14.975 | 18.078 | 1.00 | 7.09 | B | C |
| ATOM | 5161 | CG1 | ILE H | 261 | −9.037 | 14.528 | 16.715 | 1.00 | 7.46 | B | C |
| ATOM | 5162 | CD1 | ILE H | 261 | −8.132 | 13.315 | 16.782 | 1.00 | 8.38 | B | C |
| ATOM | 5163 | CG2 | ILE H | 261 | −11.024 | 14.579 | 18.262 | 1.00 | 6.61 | B | C |
| ATOM | 5164 | C | ILE H | 261 | −10.352 | 16.980 | 19.322 | 1.00 | 8.01 | B | C |
| ATOM | 5165 | O | ILE H | 261 | −11.255 | 17.770 | 19.051 | 1.00 | 8.26 | B | O |
| ATOM | 5166 | N | ASN H | 262 | −10.081 | 16.586 | 20.560 | 1.00 | 9.05 | B | N |
| ATOM | 5167 | CA | ASN H | 262 | −10.896 | 17.006 | 21.689 | 1.00 | 9.88 | B | C |
| ATOM | 5168 | CB | ASN H | 262 | −10.175 | 16.714 | 23.001 | 1.00 | 9.99 | B | C |
| ATOM | 5169 | CG | ASN H | 262 | −11.127 | 16.353 | 24.111 | 1.00 | 11.16 | B | C |
| ATOM | 5170 | OD1 | ASN H | 262 | −12.345 | 16.408 | 23.936 | 1.00 | 12.70 | B | O |
| ATOM | 5171 | ND2 | ASN H | 262 | −10.583 | 15.980 | 25.264 | 1.00 | 13.20 | B | N |
| ATOM | 5172 | C | ASN H | 262 | −11.258 | 18.482 | 21.619 | 1.00 | 10.17 | B | C |
| ATOM | 5173 | O | ASN H | 262 | −12.347 | 18.887 | 22.027 | 1.00 | 10.52 | B | O |
| ATOM | 5174 | N | ASP H | 263 | −10.310 | 19.289 | 21.158 | 1.00 | 10.62 | B | N |
| ATOM | 5175 | CA | ASP H | 263 | −10.437 | 20.734 | 21.247 | 1.00 | 11.02 | B | C |
| ATOM | 5176 | CB | ASP H | 263 | −9.057 | 21.395 | 21.285 | 1.00 | 11.49 | B | C |
| ATOM | 5177 | CG | ASP H | 263 | −9.138 | 22.905 | 21.401 | 1.00 | 12.80 | B | C |
| ATOM | 5178 | OD1 | ASP H | 263 | −9.972 | 23.401 | 22.188 | 1.00 | 14.72 | B | O |
| ATOM | 5179 | OD2 | ASP H | 263 | −8.354 | 23.596 | 20.717 | 1.00 | 13.88 | B | O |
| ATOM | 5180 | C | ASP H | 263 | −11.261 | 21.293 | 20.091 | 1.00 | 10.92 | B | C |
| ATOM | 5181 | O | ASP H | 263 | −11.686 | 22.446 | 20.130 | 1.00 | 10.96 | B | O |
| ATOM | 5182 | N | MET H | 264 | −11.539 | 20.453 | 19.097 | 1.00 | 10.91 | B | N |
| ATOM | 5183 | CA | MET H | 264 | −12.199 | 20.904 | 17.871 | 1.00 | 10.78 | B | C |
| ATOM | 5184 | CB | MET H | 264 | −12.091 | 19.840 | 16.770 | 1.00 | 10.51 | B | C |
| ATOM | 5185 | CG | MET H | 264 | −10.664 | 19.427 | 16.425 | 1.00 | 11.26 | B | C |
| ATOM | 5186 | SD | MET H | 264 | −10.510 | 18.571 | 14.840 | 1.00 | 12.70 | B | S |
| ATOM | 5187 | CE | MET H | 264 | −11.715 | 17.260 | 15.021 | 1.00 | 13.92 | B | C |
| ATOM | 5188 | C | MET H | 264 | −13.666 | 21.240 | 18.119 | 1.00 | 10.85 | B | C |
| ATOM | 5189 | O | MET H | 264 | −14.369 | 20.489 | 18.795 | 1.00 | 11.03 | B | O |
| ATOM | 5190 | N | PRO H | 265 | −14.146 | 22.342 | 17.517 | 1.00 | 10.88 | B | N |
| ATOM | 5191 | CA | PRO H | 265 | −15.522 | 22.802 | 17.700 | 1.00 | 11.09 | B | C |
| ATOM | 5192 | CB | PRO H | 265 | −15.515 | 24.198 | 17.060 | 1.00 | 11.28 | B | C |
| ATOM | 5193 | CG | PRO H | 265 | −14.322 | 24.216 | 16.167 | 1.00 | 11.24 | B | C |
| ATOM | 5194 | CD | PRO H | 265 | −13.313 | 23.356 | 16.849 | 1.00 | 11.03 | B | C |
| ATOM | 5195 | C | PRO H | 265 | −16.546 | 21.912 | 17.003 | 1.00 | 11.19 | B | C |
| ATOM | 5196 | O | PRO H | 265 | −17.320 | 22.395 | 16.177 | 1.00 | 11.47 | B | O |
| ATOM | 5197 | N | ILE H | 266 | −16.633 | 20.655 | 17.425 | 1.00 | 11.54 | B | N |
| ATOM | 5198 | CA | ILE H | 266 | −17.540 | 19.710 | 16.786 | 1.00 | 12.15 | B | C |
| ATOM | 5199 | CB | ILE H | 266 | −16.794 | 18.757 | 15.828 | 1.00 | 12.22 | B | C |
| ATOM | 5200 | CG1 | ILE H | 266 | −15.611 | 18.088 | 16.536 | 1.00 | 12.53 | B | C |
| ATOM | 5201 | CD1 | ILE H | 266 | −15.044 | 16.895 | 15.789 | 1.00 | 12.40 | B | C |
| ATOM | 5202 | CG2 | ILE H | 266 | −16.337 | 19.507 | 14.588 | 1.00 | 12.74 | B | C |
| ATOM | 5203 | C | ILE H | 266 | −18.353 | 18.902 | 17.793 | 1.00 | 12.29 | B | C |
| ATOM | 5204 | O | ILE H | 266 | −18.068 | 18.913 | 18.991 | 1.00 | 12.36 | B | O |
| ATOM | 5205 | N | THR H | 267 | −19.404 | 18.252 | 17.303 | 1.00 | 12.29 | B | N |
| ATOM | 5206 | CA | THR H | 267 | −20.248 | 17.414 | 18.148 | 1.00 | 12.43 | B | C |
| ATOM | 5207 | CB | THR H | 267 | −21.440 | 16.821 | 17.366 | 1.00 | 12.40 | B | C |
| ATOM | 5208 | OG1 | THR H | 267 | −21.299 | 15.397 | 17.278 | 1.00 | 13.21 | B | O |
| ATOM | 5209 | CG2 | THR H | 267 | −21.509 | 17.405 | 15.968 | 1.00 | 12.21 | B | C |
| ATOM | 5210 | C | THR H | 267 | −19.432 | 16.276 | 18.740 | 1.00 | 12.37 | B | C |
| ATOM | 5211 | O | THR H | 267 | −18.246 | 16.134 | 18.446 | 1.00 | 12.29 | B | O |
| ATOM | 5212 | N | ASN H | 268 | −20.058 | 15.490 | 19.605 | 1.00 | 12.47 | B | N |
| ATOM | 5213 | CA | ASN H | 268 | −19.325 | 14.501 | 20.377 | 1.00 | 12.57 | B | C |
| ATOM | 5214 | CB | ASN H | 268 | −20.025 | 14.218 | 21.701 | 1.00 | 12.69 | B | C |
| ATOM | 5215 | CG | ASN H | 268 | −19.461 | 15.041 | 22.832 | 1.00 | 13.65 | B | C |
| ATOM | 5216 | OD1 | ASN H | 268 | −18.555 | 15.851 | 22.631 | 1.00 | 14.37 | B | O |
| ATOM | 5217 | ND2 | ASN H | 268 | −20.005 | 14.856 | 24.028 | 1.00 | 15.74 | B | N |
| ATOM | 5218 | C | ASN H | 268 | −19.084 | 13.207 | 19.621 | 1.00 | 12.43 | B | C |
| ATOM | 5219 | O | ASN H | 268 | −18.038 | 12.576 | 19.776 | 1.00 | 12.51 | B | O |
| ATOM | 5220 | N | ASP H | 269 | −20.107 | 12.747 | 18.912 | 1.00 | 12.34 | B | N |
| ATOM | 5221 | CA | ASP H | 269 | −19.976 | 11.565 | 18.077 | 1.00 | 12.33 | B | C |
| ATOM | 5222 | CB | ASP H | 269 | −21.316 | 11.222 | 17.431 | 1.00 | 12.89 | B | C |
| ATOM | 5223 | CG | ASP H | 269 | −22.404 | 10.959 | 18.456 | 1.00 | 14.84 | B | C |
| ATOM | 5224 | OD1 | ASP H | 269 | −22.145 | 10.209 | 19.422 | 1.00 | 16.90 | B | O |
| ATOM | 5225 | OD2 | ASP H | 269 | −23.487 | 11.573 | 18.346 | 1.00 | 17.65 | B | O |
| ATOM | 5226 | C | ASP H | 269 | −18.916 | 11.780 | 17.008 | 1.00 | 11.60 | B | C |
| ATOM | 5227 | O | ASP H | 269 | −18.073 | 10.914 | 16.774 | 1.00 | 11.65 | B | O |
| ATOM | 5228 | N | GLN H | 270 | −18.924 | 12.963 | 16.405 | 1.00 | 10.62 | B | N |
| ATOM | 5229 | CA | GLN H | 270 | −17.836 | 13.371 | 15.530 | 1.00 | 10.01 | B | C |
| ATOM | 5230 | CB | GLN H | 270 | −17.958 | 14.851 | 15.179 | 1.00 | 10.18 | B | C |
| ATOM | 5231 | CG | GLN H | 270 | −17.709 | 15.154 | 13.718 | 1.00 | 11.49 | B | C |
| ATOM | 5232 | CD | GLN H | 270 | −18.572 | 16.286 | 13.213 | 1.00 | 14.38 | B | C |
| ATOM | 5233 | OE1 | GLN H | 270 | −18.076 | 17.238 | 12.613 | 1.00 | 16.38 | B | O |
| ATOM | 5234 | NE2 | GLN H | 270 | −19.866 | 16.215 | 13.496 | 1.00 | 15.41 | B | N |
| ATOM | 5235 | C | GLN H | 270 | −16.486 | 13.097 | 16.181 | 1.00 | 9.48 | B | C |
| ATOM | 5236 | O | GLN H | 270 | −15.696 | 12.296 | 15.682 | 1.00 | 9.12 | B | O |
| ATOM | 5237 | N | LYS H | 271 | −16.229 | 13.763 | 17.301 | 1.00 | 9.19 | B | N |
| ATOM | 5238 | CA | LYS H | 271 | −15.004 | 13.541 | 18.056 | 1.00 | 8.89 | B | C |
| ATOM | 5239 | CB | LYS H | 271 | −15.117 | 14.168 | 19.448 | 1.00 | 8.52 | B | C |
| ATOM | 5240 | CG | LYS H | 271 | −15.410 | 15.663 | 19.445 | 1.00 | 9.14 | B | C |
| ATOM | 5241 | CD | LYS H | 271 | −14.906 | 16.316 | 20.726 | 1.00 | 10.72 | B | C |
| ATOM | 5242 | CE | LYS H | 271 | −15.469 | 17.718 | 20.900 | 1.00 | 10.79 | B | C |
| ATOM | 5243 | NZ | LYS H | 271 | −14.711 | 18.506 | 21.916 | 1.00 | 9.77 | B | N |
| ATOM | 5244 | C | LYS H | 271 | −14.699 | 12.049 | 18.175 | 1.00 | 8.83 | B | C |
| ATOM | 5245 | O | LYS H | 271 | −13.660 | 11.577 | 17.713 | 1.00 | 9.05 | B | O |
| ATOM | 5246 | N | LYS H | 272 | −15.653 | 11.301 | 18.719 | 1.00 | 8.65 | B | N |
| ATOM | 5247 | CA | LYS H | 272 | −15.485 | 9.868 | 18.899 | 1.00 | 8.50 | B | C |
| ATOM | 5248 | CB | LYS H | 272 | −16.766 | 9.240 | 19.443 | 1.00 | 9.14 | B | C |
| ATOM | 5249 | CG | LYS H | 272 | −16.762 | 7.726 | 19.397 | 1.00 | 11.58 | B | C |
| ATOM | 5250 | CD | LYS H | 272 | −18.170 | 7.167 | 19.394 | 1.00 | 15.78 | B | C |
| ATOM | 5251 | CE | LYS H | 272 | −18.151 | 5.663 | 19.599 | 1.00 | 17.65 | B | C |
| ATOM | 5252 | NZ | LYS H | 272 | −19.476 | 5.142 | 20.029 | 1.00 | 19.68 | B | N |
| ATOM | 5253 | C | LYS H | 272 | −15.083 | 9.185 | 17.598 | 1.00 | 7.88 | B | C |
| ATOM | 5254 | O | LYS H | 272 | −14.195 | 8.332 | 17.587 | 1.00 | 8.12 | B | O |
| ATOM | 5255 | N | LEU H | 273 | −15.787 | 9.506 | 16.517 | 1.00 | 6.95 | B | N |
| ATOM | 5256 | CA | LEU H | 273 | −15.456 | 8.939 | 15.216 | 1.00 | 5.70 | B | C |
| ATOM | 5257 | CB | LEU H | 273 | −16.366 | 9.490 | 14.116 | 1.00 | 5.85 | B | C |
| ATOM | 5258 | CG | LEU H | 273 | −15.894 | 9.204 | 12.683 | 1.00 | 4.65 | B | C |
| ATOM | 5259 | CD1 | LEU H | 273 | −16.085 | 7.739 | 12.312 | 1.00 | 4.57 | B | C |
| ATOM | 5260 | CD2 | LEU H | 273 | −16.569 | 10.123 | 11.669 | 1.00 | 3.69 | B | C |
| ATOM | 5261 | C | LEU H | 273 | −14.005 | 9.227 | 14.877 | 1.00 | 5.14 | B | C |
| ATOM | 5262 | O | LEU H | 273 | −13.188 | 8.312 | 14.795 | 1.00 | 5.25 | B | O |
| ATOM | 5263 | N | MET H | 274 | −13.668 | 10.509 | 14.775 | 1.00 | 4.52 | B | N |
| ATOM | 5264 | CA | MET H | 274 | −12.307 | 10.910 | 14.447 | 1.00 | 4.59 | B | C |
| ATOM | 5265 | CB | MET H | 274 | −12.147 | 12.429 | 14.567 | 1.00 | 4.35 | B | C |
| ATOM | 5266 | CG | MET H | 274 | −12.862 | 13.219 | 13.479 | 1.00 | 4.50 | B | C |
| ATOM | 5267 | SD | MET H | 274 | −12.547 | 14.992 | 13.574 | 1.00 | 5.51 | B | S |
| ATOM | 5268 | CE | MET H | 274 | −10.875 | 15.078 | 12.935 | 1.00 | 5.26 | B | C |
| ATOM | 5269 | C | MET H | 274 | −11.294 | 10.190 | 15.334 | 1.00 | 5.18 | B | C |
| ATOM | 5270 | O | MET H | 274 | −10.272 | 9.703 | 14.851 | 1.00 | 5.61 | B | O |
| ATOM | 5271 | N | SER H | 275 | −11.652 | 10.006 | 16.601 | 1.00 | 5.89 | B | N |
| ATOM | 5272 | CA | SER H | 275 | −10.752 | 9.380 | 17.557 | 1.00 | 6.18 | B | C |
| ATOM | 5273 | CB | SER H | 275 | −11.237 | 9.619 | 18.988 | 1.00 | 6.33 | B | C |
| ATOM | 5274 | OG | SER H | 275 | −11.029 | 10.969 | 19.371 | 1.00 | 5.91 | B | O |
| ATOM | 5275 | C | SER H | 275 | −10.583 | 7.886 | 17.288 | 1.00 | 6.58 | B | C |
| ATOM | 5276 | O | SER H | 275 | −9.519 | 7.318 | 17.544 | 1.00 | 7.03 | B | O |
| ATOM | 5277 | N | ASN H | 276 | −11.607 | 7.268 | 16.711 | 1.00 | 7.07 | B | N |
| ATOM | 5278 | CA | ASN H | 276 | −11.525 | 5.857 | 16.369 | 1.00 | 7.86 | B | C |
| ATOM | 5279 | CB | ASN H | 276 | −12.885 | 5.173 | 16.547 | 1.00 | 8.34 | B | C |
| ATOM | 5280 | CG | ASN H | 276 | −13.108 | 4.676 | 17.969 | 1.00 | 9.77 | B | C |
| ATOM | 5281 | OD1 | ASN H | 276 | −12.161 | 4.307 | 18.666 | 1.00 | 11.84 | B | O |
| ATOM | 5282 | ND2 | ASN H | 276 | −14.351 | 4.748 | 18.433 | 1.00 | 11.15 | B | N |
| ATOM | 5283 | C | ASN H | 276 | −10.959 | 5.605 | 14.971 | 1.00 | 7.71 | B | C |
| ATOM | 5284 | O | ASN H | 276 | −10.950 | 4.468 | 14.498 | 1.00 | 7.84 | B | O |
| ATOM | 5285 | N | ASN H | 277 | −10.386 | 6.643 | 14.363 | 1.00 | 7.77 | B | N |
| ATOM | 5286 | CA | ASN H | 277 | −10.026 | 6.597 | 12.943 | 1.00 | 7.85 | B | C |
| ATOM | 5287 | CB | ASN H | 277 | −11.209 | 7.021 | 12.073 | 1.00 | 7.58 | B | C |
| ATOM | 5288 | CG | ASN H | 277 | −12.197 | 5.896 | 11.846 | 1.00 | 8.29 | B | C |
| ATOM | 5289 | OD1 | ASN H | 277 | −11.932 | 4.970 | 11.079 | 1.00 | 9.04 | B | O |
| ATOM | 5290 | ND2 | ASN H | 277 | −13.297 | 5.915 | 12.592 | 1.00 | 9.57 | B | N |
| ATOM | 5291 | C | ASN H | 277 | −8.792 | 7.426 | 12.585 | 1.00 | 7.86 | B | C |
| ATOM | 5292 | O | ASN H | 277 | −8.614 | 7.832 | 11.435 | 1.00 | 7.85 | B | O |
| ATOM | 5293 | N | VAL H | 278 | −8.003 | 7.754 | 13.603 | 1.00 | 7.67 | B | N |
| ATOM | 5294 | CA | VAL H | 278 | −6.682 | 8.354 | 13.425 | 1.00 | 7.77 | B | C |
| ATOM | 5295 | CB | VAL H | 278 | −5.677 | 7.813 | 14.459 | 1.00 | 7.98 | B | C |
| ATOM | 5296 | CG1 | VAL H | 278 | −5.763 | 8.615 | 15.749 | 1.00 | 7.21 | B | C |
| ATOM | 5297 | CG2 | VAL H | 278 | −5.932 | 6.336 | 14.725 | 1.00 | 8.99 | B | C |
| ATOM | 5298 | C | VAL H | 278 | −6.095 | 8.223 | 12.019 | 1.00 | 7.76 | B | C |
| ATOM | 5299 | O | VAL H | 278 | −5.641 | 9.211 | 11.444 | 1.00 | 7.84 | B | O |
| ATOM | 5300 | N | GLN H | 279 | −5.935 | 6.989 | 11.553 | 1.00 | 7.47 | B | N |
| ATOM | 5301 | CA | GLN H | 279 | −5.162 | 6.725 | 10.343 | 1.00 | 7.07 | B | C |
| ATOM | 5302 | CB | GLN H | 279 | −5.291 | 5.256 | 9.938 | 1.00 | 7.62 | B | C |
| ATOM | 5303 | CG | GLN H | 279 | −3.986 | 4.621 | 9.497 | 1.00 | 10.35 | B | C |
| ATOM | 5304 | CD | GLN H | 279 | −4.194 | 3.335 | 8.725 | 1.00 | 13.70 | B | C |
| ATOM | 5305 | OE1 | GLN H | 279 | −5.319 | 2.986 | 8.367 | 1.00 | 15.06 | B | O |
| ATOM | 5306 | NE2 | GLN H | 279 | −3.105 | 2.622 | 8.460 | 1.00 | 13.97 | B | N |
| ATOM | 5307 | C | GLN H | 279 | −5.610 | 7.621 | 9.192 | 1.00 | 6.02 | B | C |
| ATOM | 5308 | O | GLN H | 279 | −4.795 | 8.297 | 8.563 | 1.00 | 5.45 | B | O |
| ATOM | 5309 | N | ILE H | 280 | −6.881 | 7.492 | 8.829 | 1.00 | 5.25 | B | N |
| ATOM | 5310 | CA | ILE H | 280 | −7.499 | 8.346 | 7.822 | 1.00 | 4.74 | B | C |
| ATOM | 5311 | CB | ILE H | 280 | −9.022 | 8.133 | 7.775 | 1.00 | 4.58 | B | C |
| ATOM | 5312 | CG1 | ILE H | 280 | −9.349 | 6.641 | 7.685 | 1.00 | 4.16 | B | C |
| ATOM | 5313 | CD1 | ILE H | 280 | −8.986 | 6.014 | 6.357 | 1.00 | 3.88 | B | C |
| ATOM | 5314 | CG2 | ILE H | 280 | −9.636 | 8.897 | 6.612 | 1.00 | 3.92 | B | C |
| ATOM | 5315 | C | ILE H | 280 | −7.225 | 9.818 | 8.101 | 1.00 | 4.43 | B | C |
| ATOM | 5316 | O | ILE H | 280 | −6.655 | 10.522 | 7.268 | 1.00 | 4.39 | B | O |
| ATOM | 5317 | N | VAL H | 281 | −7.745 | 10.301 | 9.224 | 1.00 | 4.05 | B | N |
| ATOM | 5318 | CA | VAL H | 281 | −7.502 | 11.671 | 9.651 | 1.00 | 3.58 | B | C |
| ATOM | 5319 | CB | VAL H | 281 | −7.705 | 11.833 | 11.170 | 1.00 | 3.13 | B | C |
| ATOM | 5320 | CG1 | VAL H | 281 | −7.901 | 13.299 | 11.526 | 1.00 | 2.34 | B | C |
| ATOM | 5321 | CG2 | VAL H | 281 | −8.890 | 11.002 | 11.638 | 1.00 | 2.21 | B | C |
| ATOM | 5322 | C | VAL H | 281 | −6.089 | 12.110 | 9.280 | 1.00 | 3.96 | B | C |
| ATOM | 5323 | O | VAL H | 281 | −5.905 | 13.063 | 8.522 | 1.00 | 4.15 | B | O |
| ATOM | 5324 | N | ARG H | 282 | −5.096 | 11.377 | 9.777 | 1.00 | 4.23 | B | N |
| ATOM | 5325 | CA | ARG H | 282 | −3.697 | 11.649 | 9.454 | 1.00 | 4.45 | B | C |
| ATOM | 5326 | CB | ARG H | 282 | −2.799 | 10.506 | 9.941 | 1.00 | 3.96 | B | C |
| ATOM | 5327 | CG | ARG H | 282 | −2.843 | 10.255 | 11.440 | 1.00 | 2.99 | B | C |
| ATOM | 5328 | CD | ARG H | 282 | −1.441 | 10.173 | 12.028 | 1.00 | 2.28 | B | C |
| ATOM | 5329 | NE | ARG H | 282 | −1.470 | 9.891 | 13.460 | 1.00 | 2.00 | B | N |
| ATOM | 5330 | CZ | ARG H | 282 | −0.687 | 10.483 | 14.358 | 1.00 | 2.44 | B | C |
| ATOM | 5331 | NH1 | ARG H | 282 | 0.219 | 11.371 | 13.970 | 1.00 | 2.04 | B | N |
| ATOM | 5332 | NH2 | ARG H | 282 | −0.770 | 10.140 | 15.636 | 1.00 | 3.44 | B | N |
| ATOM | 5333 | C | ARG H | 282 | −3.530 | 11.813 | 7.953 | 1.00 | 5.32 | B | C |
| ATOM | 5334 | O | ARG H | 282 | −2.952 | 12.793 | 7.482 | 1.00 | 5.95 | B | O |
| ATOM | 5335 | N | GLN H | 283 | −3.946 | 10.791 | 7.214 | 1.00 | 5.50 | B | N |
| ATOM | 5336 | CA | GLN H | 283 | −3.837 | 10.802 | 5.766 | 1.00 | 5.80 | B | C |
| ATOM | 5337 | CB | GLN H | 283 | −4.404 | 9.511 | 5.178 | 1.00 | 6.19 | B | C |
| ATOM | 5338 | CG | GLN H | 283 | −3.531 | 8.297 | 5.431 | 1.00 | 9.68 | B | C |
| ATOM | 5339 | CD | GLN H | 283 | −4.248 | 6.994 | 5.156 | 1.00 | 13.92 | B | C |
| ATOM | 5340 | OE1 | GLN H | 283 | −5.408 | 6.984 | 4.745 | 1.00 | 16.20 | B | O |
| ATOM | 5341 | NE2 | GLN H | 283 | −3.549 | 5.883 | 5.354 | 1.00 | 14.17 | B | N |
| ATOM | 5342 | C | GLN H | 283 | −4.552 | 12.008 | 5.175 | 1.00 | 5.44 | B | C |
| ATOM | 5343 | O | GLN H | 283 | −4.187 | 12.493 | 4.104 | 1.00 | 5.43 | B | O |
| ATOM | 5344 | N | GLN H | 284 | −5.532 | 12.527 | 5.905 | 1.00 | 5.26 | B | N |
| ATOM | 5345 | CA | GLN H | 284 | −6.361 | 13.609 | 5.396 | 1.00 | 5.18 | B | C |
| ATOM | 5346 | CB | GLN H | 284 | −7.799 | 13.451 | 5.877 | 1.00 | 5.32 | B | C |
| ATOM | 5347 | CG | GLN H | 284 | −8.666 | 12.649 | 4.933 | 1.00 | 6.85 | B | C |
| ATOM | 5348 | CD | GLN H | 284 | −10.091 | 12.547 | 5.415 | 1.00 | 9.21 | B | C |
| ATOM | 5349 | OE1 | GLN H | 284 | −10.340 | 12.297 | 6.595 | 1.00 | 9.74 | B | O |
| ATOM | 5350 | NE2 | GLN H | 284 | −11.034 | 12.860 | 4.537 | 1.00 | 9.79 | B | N |
| ATOM | 5351 | C | GLN H | 284 | −5.817 | 14.967 | 5.813 | 1.00 | 4.67 | B | C |
| ATOM | 5352 | O | GLN H | 284 | −6.336 | 16.006 | 5.406 | 1.00 | 4.19 | B | O |
| ATOM | 5353 | N | SER H | 285 | −4.758 | 14.950 | 6.614 | 1.00 | 4.51 | B | N |
| ATOM | 5354 | CA | SER H | 285 | −4.240 | 16.170 | 7.212 | 1.00 | 4.39 | B | C |
| ATOM | 5355 | CB | SER H | 285 | −4.020 | 15.979 | 8.713 | 1.00 | 4.13 | B | C |
| ATOM | 5356 | OG | SER H | 285 | −5.177 | 15.446 | 9.334 | 1.00 | 4.40 | B | O |
| ATOM | 5357 | C | SER H | 285 | −2.943 | 16.598 | 6.540 | 1.00 | 4.44 | B | C |
| ATOM | 5358 | O | SER H | 285 | −2.286 | 15.804 | 5.866 | 1.00 | 4.82 | B | O |
| ATOM | 5359 | N | TYR H | 286 | −2.600 | 17.870 | 6.704 | 1.00 | 4.53 | B | N |
| ATOM | 5360 | CA | TYR H | 286 | −1.328 | 18.388 | 6.228 | 1.00 | 4.80 | B | C |
| ATOM | 5361 | CB | TYR H | 286 | −1.560 | 19.527 | 5.236 | 1.00 | 4.88 | B | C |
| ATOM | 5362 | CG | TYR H | 286 | −2.163 | 19.083 | 3.923 | 1.00 | 5.68 | B | C |
| ATOM | 5363 | CD1 | TYR H | 286 | −3.448 | 18.558 | 3.868 | 1.00 | 7.34 | B | C |
| ATOM | 5364 | CE1 | TYR H | 286 | −4.003 | 18.153 | 2.668 | 1.00 | 9.02 | B | C |
| ATOM | 5365 | CZ | TYR H | 286 | −3.279 | 18.287 | 1.502 | 1.00 | 9.40 | B | C |
| ATOM | 5366 | OH | TYR H | 286 | −3.829 | 17.892 | 0.304 | 1.00 | 10.44 | B | O |
| ATOM | 5367 | CE2 | TYR H | 286 | −2.006 | 18.817 | 1.530 | 1.00 | 9.38 | B | C |
| ATOM | 5368 | CD2 | TYR H | 286 | −1.458 | 19.214 | 2.735 | 1.00 | 7.39 | B | C |
| ATOM | 5369 | C | TYR H | 286 | −0.501 | 18.884 | 7.404 | 1.00 | 4.76 | B | C |
| ATOM | 5370 | O | TYR H | 286 | −1.036 | 19.160 | 8.478 | 1.00 | 5.25 | B | O |
| ATOM | 5371 | N | SER H | 287 | 0.803 | 19.011 | 7.194 | 1.00 | 4.52 | B | N |
| ATOM | 5372 | CA | SER H | 287 | 1.649 | 19.738 | 8.125 | 1.00 | 4.45 | B | C |
| ATOM | 5373 | CB | SER H | 287 | 2.518 | 18.768 | 8.927 | 1.00 | 4.57 | B | C |
| ATOM | 5374 | OG | SER H | 287 | 3.602 | 19.441 | 9.544 | 1.00 | 4.38 | B | O |
| ATOM | 5375 | C | SER H | 287 | 2.515 | 20.758 | 7.396 | 1.00 | 4.61 | B | C |
| ATOM | 5376 | O | SER H | 287 | 3.264 | 20.409 | 6.483 | 1.00 | 4.40 | B | O |
| ATOM | 5377 | N | ILE H | 288 | 2.294 | 22.033 | 7.710 | 1.00 | 5.11 | B | N |
| ATOM | 5378 | CA | ILE H | 288 | 3.067 | 23.130 | 7.131 | 1.00 | 5.81 | B | C |
| ATOM | 5379 | CB | ILE H | 288 | 2.176 | 24.349 | 6.825 | 1.00 | 5.52 | B | C |
| ATOM | 5380 | CG1 | ILE H | 288 | 0.730 | 23.913 | 6.578 | 1.00 | 5.47 | B | C |
| ATOM | 5381 | CD1 | ILE H | 288 | 0.479 | 23.356 | 5.196 | 1.00 | 6.23 | B | C |
| ATOM | 5382 | CG2 | ILE H | 288 | 2.733 | 25.142 | 5.651 | 1.00 | 6.22 | B | C |
| ATOM | 5383 | C | ILE H | 288 | 4.170 | 23.581 | 8.080 | 1.00 | 6.71 | B | C |
| ATOM | 5384 | O | ILE H | 288 | 3.902 | 23.948 | 9.224 | 1.00 | 7.30 | B | O |
| ATOM | 5385 | N | MET H | 289 | 5.380 | 23.699 | 7.548 | 1.00 | 7.71 | B | N |
| ATOM | 5386 | CA | MET H | 289 | 6.487 | 24.307 | 8.274 | 1.00 | 8.57 | B | C |
| ATOM | 5387 | CB | MET H | 289 | 7.781 | 24.096 | 7.492 | 1.00 | 8.71 | B | C |
| ATOM | 5388 | CG | MET H | 289 | 9.038 | 24.181 | 8.326 | 1.00 | 9.70 | B | C |
| ATOM | 5389 | SD | MET H | 289 | 10.511 | 24.120 | 7.292 | 1.00 | 12.59 | B | S |
| ATOM | 5390 | CE | MET H | 289 | 10.168 | 25.446 | 6.138 | 1.00 | 12.41 | B | C |
| ATOM | 5391 | C | MET H | 289 | 6.245 | 25.801 | 8.468 | 1.00 | 9.12 | B | C |
| ATOM | 5392 | O | MET H | 289 | 5.976 | 26.513 | 7.501 | 1.00 | 9.31 | B | O |
| ATOM | 5393 | N | SER H | 290 | 6.457 | 26.293 | 9.688 | 1.00 | 9.80 | B | N |
| ATOM | 5394 | CA | SER H | 290 | 6.140 | 27.688 | 10.021 | 1.00 | 10.49 | B | C |
| ATOM | 5395 | CB | SER H | 290 | 5.283 | 27.777 | 11.287 | 1.00 | 10.64 | B | C |
| ATOM | 5396 | OG | SER H | 290 | 4.906 | 29.117 | 11.560 | 1.00 | 11.99 | B | O |
| ATOM | 5397 | C | SER H | 290 | 7.372 | 28.582 | 10.146 | 1.00 | 10.75 | B | C |
| ATOM | 5398 | O | SER H | 290 | 7.635 | 29.401 | 9.265 | 1.00 | 10.89 | B | O |
| ATOM | 5399 | N | ILE H | 291 | 8.095 | 28.469 | 11.258 | 1.00 | 11.04 | B | N |
| ATOM | 5400 | CA | ILE H | 291 | 9.405 | 29.112 | 11.361 | 1.00 | 11.41 | B | C |
| ATOM | 5401 | CB | ILE H | 291 | 9.335 | 30.523 | 11.981 | 1.00 | 11.80 | B | C |
| ATOM | 5402 | CG1 | ILE H | 291 | 8.071 | 30.680 | 12.828 | 1.00 | 11.81 | B | C |
| ATOM | 5403 | CD1 | ILE H | 291 | 8.273 | 30.362 | 14.291 | 1.00 | 11.76 | B | C |
| ATOM | 5404 | CG2 | ILE H | 291 | 9.390 | 31.586 | 10.891 | 1.00 | 12.39 | B | C |
| ATOM | 5405 | C | ILE H | 291 | 10.510 | 28.289 | 12.022 | 1.00 | 11.13 | B | C |
| ATOM | 5406 | O | ILE H | 291 | 10.337 | 27.742 | 13.111 | 1.00 | 10.55 | B | O |
| ATOM | 5407 | N | ILE H | 292 | 11.678 | 28.302 | 11.388 | 1.00 | 11.36 | B | N |
| ATOM | 5408 | CA | ILE H | 292 | 12.827 | 27.544 | 11.855 | 1.00 | 11.82 | B | C |
| ATOM | 5409 | CB | ILE H | 292 | 13.483 | 26.752 | 10.710 | 1.00 | 11.86 | B | C |
| ATOM | 5410 | CG1 | ILE H | 292 | 12.770 | 27.030 | 9.386 | 1.00 | 13.42 | B | C |
| ATOM | 5411 | CD1 | ILE H | 292 | 11.373 | 27.593 | 9.542 | 1.00 | 15.86 | B | C |
| ATOM | 5412 | CG2 | ILE H | 292 | 13.500 | 25.262 | 11.022 | 1.00 | 10.85 | B | C |
| ATOM | 5413 | C | ILE H | 292 | 13.867 | 28.476 | 12.454 | 1.00 | 11.72 | B | C |
| ATOM | 5414 | O | ILE H | 292 | 14.414 | 29.343 | 11.771 | 1.00 | 11.28 | B | O |
| ATOM | 5415 | N | LYS H | 293 | 14.150 | 28.269 | 13.733 | 1.00 | 12.11 | B | N |
| ATOM | 5416 | CA | LYS H | 293 | 15.287 | 28.894 | 14.382 | 1.00 | 12.92 | B | C |
| ATOM | 5417 | CB | LYS H | 293 | 14.812 | 29.763 | 15.544 | 1.00 | 13.15 | B | C |
| ATOM | 5418 | CG | LYS H | 293 | 13.615 | 30.634 | 15.208 | 1.00 | 14.17 | B | C |
| ATOM | 5419 | CD | LYS H | 293 | 13.793 | 32.037 | 15.758 | 1.00 | 15.37 | B | C |
| ATOM | 5420 | CE | LYS H | 293 | 12.940 | 33.041 | 15.001 | 1.00 | 16.68 | B | C |
| ATOM | 5421 | NZ | LYS H | 293 | 13.111 | 34.421 | 15.532 | 1.00 | 18.18 | B | N |
| ATOM | 5422 | C | LYS H | 293 | 16.224 | 27.813 | 14.894 | 1.00 | 13.32 | B | C |
| ATOM | 5423 | O | LYS H | 293 | 15.800 | 26.689 | 15.167 | 1.00 | 13.17 | B | O |
| ATOM | 5424 | N | GLU H | 294 | 17.511 | 28.132 | 14.944 | 1.00 | 14.07 | B | N |
| ATOM | 5425 | CA | GLU H | 294 | 18.518 | 27.147 | 15.302 | 1.00 | 15.07 | B | C |
| ATOM | 5426 | CB | GLU H | 294 | 19.847 | 27.831 | 15.624 | 1.00 | 15.94 | B | C |
| ATOM | 5427 | CG | GLU H | 294 | 20.563 | 28.399 | 14.409 | 1.00 | 19.87 | B | C |
| ATOM | 5428 | CD | GLU H | 294 | 21.978 | 28.849 | 14.721 | 1.00 | 23.88 | B | C |
| ATOM | 5429 | OE1 | GLU H | 294 | 22.830 | 27.983 | 15.010 | 1.00 | 23.75 | B | O |
| ATOM | 5430 | OE2 | GLU H | 294 | 22.247 | 30.066 | 14.640 | 1.00 | 25.81 | B | O |
| ATOM | 5431 | C | GLU H | 294 | 18.056 | 26.299 | 16.482 | 1.00 | 14.40 | B | C |
| ATOM | 5432 | O | GLU H | 294 | 18.348 | 25.106 | 16.546 | 1.00 | 14.33 | B | O |
| ATOM | 5433 | N | GLU H | 295 | 17.279 | 26.904 | 17.378 | 1.00 | 13.97 | B | N |
| ATOM | 5434 | CA | GLU H | 295 | 16.914 | 26.255 | 18.635 | 1.00 | 13.84 | B | C |
| ATOM | 5435 | CB | GLU H | 295 | 17.570 | 26.953 | 19.830 | 1.00 | 14.67 | B | C |
| ATOM | 5436 | CG | GLU H | 295 | 18.047 | 28.366 | 19.550 | 1.00 | 17.56 | B | C |
| ATOM | 5437 | CD | GLU H | 295 | 16.928 | 29.281 | 19.096 | 1.00 | 21.41 | B | C |
| ATOM | 5438 | OE1 | GLU H | 295 | 16.816 | 29.524 | 17.876 | 1.00 | 22.35 | B | O |
| ATOM | 5439 | OE2 | GLU H | 295 | 16.190 | 29.795 | 19.962 | 1.00 | 23.20 | B | O |
| ATOM | 5440 | C | GLU H | 295 | 15.406 | 26.155 | 18.851 | 1.00 | 12.77 | B | C |
| ATOM | 5441 | O | GLU H | 295 | 14.956 | 25.743 | 19.919 | 1.00 | 12.58 | B | O |
| ATOM | 5442 | N | VAL H | 296 | 14.627 | 26.533 | 17.845 | 1.00 | 11.72 | B | N |
| ATOM | 5443 | CA | VAL H | 296 | 13.195 | 26.268 | 17.870 | 1.00 | 10.65 | B | C |
| ATOM | 5444 | CB | VAL H | 296 | 12.418 | 27.396 | 18.571 | 1.00 | 10.47 | B | C |
| ATOM | 5445 | CG1 | VAL H | 296 | 10.974 | 26.983 | 18.804 | 1.00 | 9.77 | B | C |
| ATOM | 5446 | CG2 | VAL H | 296 | 13.090 | 27.760 | 19.884 | 1.00 | 11.31 | B | C |
| ATOM | 5447 | C | VAL H | 296 | 12.632 | 26.053 | 16.473 | 1.00 | 10.26 | B | C |
| ATOM | 5448 | O | VAL H | 296 | 12.887 | 26.838 | 15.561 | 1.00 | 10.09 | B | O |
| ATOM | 5449 | N | LEU H | 297 | 11.781 | 25.042 | 16.345 | 1.00 | 9.77 | B | N |
| ATOM | 5450 | CA | LEU H | 297 | 11.034 | 24.815 | 15.119 | 1.00 | 8.97 | B | C |
| ATOM | 5451 | CB | LEU H | 297 | 11.386 | 23.448 | 14.534 | 1.00 | 8.64 | B | C |
| ATOM | 5452 | CG | LEU H | 297 | 10.442 | 22.948 | 13.442 | 1.00 | 9.01 | B | C |
| ATOM | 5453 | CD1 | LEU H | 297 | 10.243 | 24.018 | 12.373 | 1.00 | 9.79 | B | C |
| ATOM | 5454 | CD2 | LEU H | 297 | 10.960 | 21.648 | 12.834 | 1.00 | 9.43 | B | C |
| ATOM | 5455 | C | LEU H | 297 | 9.535 | 24.885 | 15.388 | 1.00 | 8.34 | B | C |
| ATOM | 5456 | O | LEU H | 297 | 9.006 | 24.129 | 16.205 | 1.00 | 8.64 | B | O |
| ATOM | 5457 | N | ALA H | 298 | 8.848 | 25.771 | 14.675 | 1.00 | 7.21 | B | N |
| ATOM | 5458 | CA | ALA H | 298 | 7.396 | 25.872 | 14.777 | 1.00 | 6.33 | B | C |
| ATOM | 5459 | CB | ALA H | 298 | 6.987 | 27.268 | 15.238 | 1.00 | 6.68 | B | C |
| ATOM | 5460 | C | ALA H | 298 | 6.749 | 25.537 | 13.441 | 1.00 | 5.85 | B | C |
| ATOM | 5461 | O | ALA H | 298 | 7.310 | 25.823 | 12.384 | 1.00 | 5.96 | B | O |
| ATOM | 5462 | N | TYR H | 299 | 5.653 | 24.793 | 13.492 | 1.00 | 5.28 | B | N |
| ATOM | 5463 | CA | TYR H | 299 | 4.928 | 24.443 | 12.282 | 1.00 | 4.63 | B | C |
| ATOM | 5464 | CB | TYR H | 299 | 5.468 | 23.142 | 11.691 | 1.00 | 4.36 | B | C |
| ATOM | 5465 | CG | TYR H | 299 | 5.432 | 21.978 | 12.652 | 1.00 | 4.34 | B | C |
| ATOM | 5466 | CD1 | TYR H | 299 | 4.330 | 21.136 | 12.711 | 1.00 | 4.48 | B | C |
| ATOM | 5467 | CE1 | TYR H | 299 | 4.299 | 20.061 | 13.578 | 1.00 | 4.55 | B | C |
| ATOM | 5468 | CZ | TYR H | 299 | 5.373 | 19.826 | 14.410 | 1.00 | 3.72 | B | C |
| ATOM | 5469 | OH | TYR H | 299 | 5.343 | 18.762 | 15.282 | 1.00 | 2.38 | B | O |
| ATOM | 5470 | CE2 | TYR H | 299 | 6.475 | 20.651 | 14.374 | 1.00 | 4.13 | B | C |
| ATOM | 5471 | CD2 | TYR H | 299 | 6.494 | 21.727 | 13.509 | 1.00 | 4.38 | B | C |
| ATOM | 5472 | C | TYR H | 299 | 3.443 | 24.308 | 12.563 | 1.00 | 4.32 | B | C |
| ATOM | 5473 | O | TYR H | 299 | 3.034 | 24.068 | 13.699 | 1.00 | 4.67 | B | O |
| ATOM | 5474 | N | VAL H | 300 | 2.638 | 24.454 | 11.519 | 1.00 | 3.87 | B | N |
| ATOM | 5475 | CA | VAL H | 300 | 1.193 | 24.395 | 11.664 | 1.00 | 3.57 | B | C |
| ATOM | 5476 | CB | VAL H | 300 | 0.501 | 25.518 | 10.873 | 1.00 | 3.52 | B | C |
| ATOM | 5477 | CG1 | VAL H | 300 | −0.990 | 25.241 | 10.751 | 1.00 | 3.44 | B | C |
| ATOM | 5478 | CG2 | VAL H | 300 | 0.743 | 26.861 | 11.544 | 1.00 | 3.90 | B | C |
| ATOM | 5479 | C | VAL H | 300 | 0.654 | 23.049 | 11.203 | 1.00 | 3.53 | B | C |
| ATOM | 5480 | O | VAL H | 300 | 0.858 | 22.647 | 10.058 | 1.00 | 3.68 | B | O |
| ATOM | 5481 | N | VAL H | 301 | −0.061 | 22.370 | 12.092 | 1.00 | 3.41 | B | N |
| ATOM | 5482 | CA | VAL H | 301 | −0.857 | 21.216 | 11.704 | 1.00 | 3.36 | B | C |
| ATOM | 5483 | CB | VAL H | 301 | −1.038 | 20.233 | 12.873 | 1.00 | 3.24 | B | C |
| ATOM | 5484 | CG1 | VAL H | 301 | −2.141 | 19.235 | 12.559 | 1.00 | 3.76 | B | C |
| ATOM | 5485 | CG2 | VAL H | 301 | 0.270 | 19.516 | 13.169 | 1.00 | 3.10 | B | C |
| ATOM | 5486 | C | VAL H | 301 | −2.223 | 21.641 | 11.183 | 1.00 | 3.48 | B | C |
| ATOM | 5487 | O | VAL H | 301 | −2.772 | 22.661 | 11.602 | 1.00 | 3.92 | B | O |
| ATOM | 5488 | N | GLN H | 302 | −2.766 | 20.845 | 10.270 | 1.00 | 3.21 | B | N |
| ATOM | 5489 | CA | GLN H | 302 | −3.989 | 21.201 | 9.569 | 1.00 | 3.01 | B | C |
| ATOM | 5490 | CB | GLN H | 302 | −3.662 | 21.767 | 8.188 | 1.00 | 3.24 | B | C |
| ATOM | 5491 | CG | GLN H | 302 | −4.875 | 21.992 | 7.307 | 1.00 | 5.21 | B | C |
| ATOM | 5492 | CD | GLN H | 302 | −4.603 | 22.977 | 6.191 | 1.00 | 7.54 | B | C |
| ATOM | 5493 | OE1 | GLN H | 302 | −3.715 | 23.822 | 6.297 | 1.00 | 7.28 | B | O |
| ATOM | 5494 | NE2 | GLN H | 302 | −5.364 | 22.870 | 5.108 | 1.00 | 9.27 | B | N |
| ATOM | 5495 | C | GLN H | 302 | −4.885 | 19.978 | 9.434 | 1.00 | 2.42 | B | C |
| ATOM | 5496 | O | GLN H | 302 | −4.564 | 19.038 | 8.706 | 1.00 | 2.40 | B | O |
| ATOM | 5497 | N | LEU H | 303 | −5.964 | 19.959 | 10.207 | 1.00 | 2.00 | B | N |
| ATOM | 5498 | CA | LEU H | 303 | −6.802 | 18.775 | 10.329 | 1.00 | 2.00 | B | C |
| ATOM | 5499 | CB | LEU H | 303 | −6.945 | 18.389 | 11.802 | 1.00 | 2.00 | B | C |
| ATOM | 5500 | CG | LEU H | 303 | −5.634 | 18.464 | 12.590 | 1.00 | 2.00 | B | C |
| ATOM | 5501 | CD1 | LEU H | 303 | −5.840 | 18.189 | 14.073 | 1.00 | 2.00 | B | C |
| ATOM | 5502 | CD2 | LEU H | 303 | −4.598 | 17.517 | 11.999 | 1.00 | 2.00 | B | C |
| ATOM | 5503 | C | LEU H | 303 | −8.172 | 19.024 | 9.704 | 1.00 | 2.00 | B | C |
| ATOM | 5504 | O | LEU H | 303 | −8.499 | 20.159 | 9.359 | 1.00 | 2.45 | B | O |
| ATOM | 5505 | N | PRO H | 304 | −8.955 | 17.949 | 9.506 | 1.00 | 2.00 | B | N |
| ATOM | 5506 | CA | PRO H | 304 | −10.222 | 18.040 | 8.763 | 1.00 | 2.07 | B | C |
| ATOM | 5507 | CB | PRO H | 304 | −10.407 | 16.623 | 8.206 | 1.00 | 2.00 | B | C |
| ATOM | 5508 | CG | PRO H | 304 | −9.560 | 15.748 | 9.074 | 1.00 | 2.00 | B | C |
| ATOM | 5509 | CD | PRO H | 304 | −8.390 | 16.589 | 9.469 | 1.00 | 2.06 | B | C |
| ATOM | 5510 | C | PRO H | 304 | −11.449 | 18.462 | 9.590 | 1.00 | 2.11 | B | C |
| ATOM | 5511 | O | PRO H | 304 | −11.353 | 18.606 | 10.812 | 1.00 | 2.13 | B | O |
| ATOM | 5512 | N | ALA H | 305 | −12.622 | 18.425 | 8.953 | 1.00 | 30.00 | B | N |
| ATOM | 5513 | CA | ALA H | 305 | −12.858 | 19.173 | 7.708 | 1.00 | 30.00 | B | C |
| ATOM | 5514 | CB | ALA H | 305 | −11.532 | 19.487 | 7.023 | 1.00 | 30.00 | B | C |
| ATOM | 5515 | C | ALA H | 305 | −13.809 | 18.445 | 6.733 | 1.00 | 30.00 | B | C |
| ATOM | 5516 | O | ALA H | 305 | −13.601 | 17.265 | 6.429 | 1.00 | 30.00 | B | O |
| ATOM | 5517 | N | TYR H | 306 | −14.925 | 19.101 | 6.370 | 1.00 | 13.49 | B | N |
| ATOM | 5518 | CA | TYR H | 306 | −15.658 | 18.816 | 5.101 | 1.00 | 13.70 | B | C |
| ATOM | 5519 | CB | TYR H | 306 | −16.947 | 18.016 | 5.303 | 1.00 | 13.93 | B | C |
| ATOM | 5520 | CG | TYR H | 306 | −17.614 | 18.033 | 6.640 | 1.00 | 14.08 | B | C |
| ATOM | 5521 | CD1 | TYR H | 306 | −18.719 | 18.859 | 6.857 | 1.00 | 14.59 | B | C |
| ATOM | 5522 | CE1 | TYR H | 306 | −19.825 | 18.384 | 7.554 | 1.00 | 14.82 | B | C |
| ATOM | 5523 | CZ | TYR H | 306 | −19.818 | 17.083 | 8.031 | 1.00 | 14.52 | B | C |
| ATOM | 5524 | OH | TYR H | 306 | −20.780 | 16.679 | 8.925 | 1.00 | 13.02 | B | O |
| ATOM | 5525 | CE2 | TYR H | 306 | −18.727 | 16.282 | 7.818 | 1.00 | 15.22 | B | C |
| ATOM | 5526 | CD2 | TYR H | 306 | −17.583 | 16.855 | 7.363 | 1.00 | 14.61 | B | C |
| ATOM | 5527 | C | TYR H | 306 | −16.111 | 19.916 | 4.144 | 1.00 | 13.68 | B | C |
| ATOM | 5528 | O | TYR H | 306 | −15.500 | 20.982 | 4.051 | 1.00 | 13.95 | B | O |
| ATOM | 5529 | N | GLY H | 307 | −17.412 | 19.756 | 3.831 | 1.00 | 13.54 | B | N |
| ATOM | 5530 | CA | GLY H | 307 | −18.190 | 20.428 | 2.747 | 1.00 | 13.37 | B | C |
| ATOM | 5531 | C | GLY H | 307 | −18.295 | 19.776 | 1.353 | 1.00 | 13.47 | B | C |
| ATOM | 5532 | O | GLY H | 307 | −17.282 | 19.701 | 0.658 | 1.00 | 13.90 | B | O |
| ATOM | 5533 | N | VAL H | 308 | −19.505 | 19.569 | 0.815 | 1.00 | 13.31 | B | N |
| ATOM | 5534 | CA | VAL H | 308 | −19.608 | 19.371 | −0.649 | 1.00 | 13.30 | B | C |
| ATOM | 5535 | CB | VAL H | 308 | −18.352 | 18.643 | −1.188 | 1.00 | 13.03 | B | C |
| ATOM | 5536 | CG1 | VAL H | 308 | −17.948 | 19.190 | −2.545 | 1.00 | 13.62 | B | C |
| ATOM | 5537 | CG2 | VAL H | 308 | −17.209 | 18.740 | −0.205 | 1.00 | 12.86 | B | C |
| ATOM | 5538 | C | VAL H | 308 | −20.720 | 18.501 | −1.196 | 1.00 | 13.58 | B | C |
| ATOM | 5539 | O | VAL H | 308 | −21.809 | 18.418 | −0.627 | 1.00 | 13.71 | B | O |
| ATOM | 5540 | N | ILE H | 309 | −20.173 | 17.489 | −1.845 | 1.00 | 4.08 | B | N |
| ATOM | 5541 | CA | ILE H | 309 | −19.600 | 16.440 | −1.057 | 1.00 | 4.84 | B | C |
| ATOM | 5542 | CB | ILE H | 309 | −19.530 | 16.902 | 0.426 | 1.00 | 4.89 | B | C |
| ATOM | 5543 | CG1 | ILE H | 309 | −18.554 | 18.070 | 0.585 | 1.00 | 5.85 | B | C |
| ATOM | 5544 | CD1 | ILE H | 309 | −17.312 | 17.711 | 1.373 | 1.00 | 8.12 | B | C |
| ATOM | 5545 | CG2 | ILE H | 309 | −19.121 | 15.756 | 1.332 | 1.00 | 4.44 | B | C |
| ATOM | 5546 | C | ILE H | 309 | −20.750 | 15.497 | −1.251 | 1.00 | 5.23 | B | C |
| ATOM | 5547 | O | ILE H | 309 | −21.670 | 15.818 | −1.998 | 1.00 | 5.92 | B | O |
| ATOM | 5548 | N | ASP H | 310 | −20.752 | 14.358 | −0.584 | 1.00 | 5.33 | B | N |
| ATOM | 5549 | CA | ASP H | 310 | −21.992 | 13.604 | −0.482 | 1.00 | 5.83 | B | C |
| ATOM | 5550 | CB | ASP H | 310 | −23.072 | 14.462 | 0.188 | 1.00 | 6.21 | B | C |
| ATOM | 5551 | CG | ASP H | 310 | −22.698 | 14.894 | 1.595 | 1.00 | 7.19 | B | C |
| ATOM | 5552 | OD1 | ASP H | 310 | −22.903 | 16.084 | 1.920 | 1.00 | 7.74 | B | O |
| ATOM | 5553 | OD2 | ASP H | 310 | −22.425 | 14.005 | 2.428 | 1.00 | 7.58 | B | O |
| ATOM | 5554 | C | ASP H | 310 | −22.524 | 13.115 | −1.841 | 1.00 | 6.02 | B | C |
| ATOM | 5555 | O | ASP H | 310 | −23.670 | 12.673 | −1.927 | 1.00 | 6.05 | B | O |
| ATOM | 5556 | N | THR H | 311 | −21.716 | 13.207 | −2.897 | 1.00 | 6.24 | B | N |
| ATOM | 5557 | CA | THR H | 311 | −22.015 | 12.488 | −4.140 | 1.00 | 6.14 | B | C |
| ATOM | 5558 | CB | THR H | 311 | −21.097 | 12.933 | −5.305 | 1.00 | 5.69 | B | C |
| ATOM | 5559 | OG1 | THR H | 311 | −20.497 | 14.196 | −4.994 | 1.00 | 5.34 | B | O |
| ATOM | 5560 | CG2 | THR H | 311 | −21.891 | 13.063 | −6.599 | 1.00 | 7.10 | B | C |
| ATOM | 5561 | C | THR H | 311 | −21.832 | 10.990 | −3.911 | 1.00 | 6.78 | B | C |
| ATOM | 5562 | O | THR H | 311 | −20.946 | 10.587 | −3.160 | 1.00 | 7.33 | B | O |
| ATOM | 5563 | N | PRO H | 312 | −22.611 | 10.155 | −4.621 | 1.00 | 7.00 | B | N |
| ATOM | 5564 | CA | PRO H | 312 | −22.351 | 8.718 | −4.567 | 1.00 | 7.23 | B | C |
| ATOM | 5565 | CB | PRO H | 312 | −23.560 | 8.115 | −5.289 | 1.00 | 7.00 | B | C |
| ATOM | 5566 | CG | PRO H | 312 | −24.053 | 9.200 | −6.178 | 1.00 | 6.59 | B | C |
| ATOM | 5567 | CD | PRO H | 312 | −23.800 | 10.475 | −5.432 | 1.00 | 6.99 | B | C |
| ATOM | 5568 | C | PRO H | 312 | −21.061 | 8.352 | −5.298 | 1.00 | 7.69 | B | C |
| ATOM | 5569 | O | PRO H | 312 | −20.796 | 8.881 | −6.377 | 1.00 | 7.55 | B | O |
| ATOM | 5570 | N | CYS H | 313 | −20.259 | 7.469 | −4.712 | 1.00 | 8.10 | B | N |
| ATOM | 5571 | CA | CYS H | 313 | −19.271 | 6.730 | −5.491 | 1.00 | 8.54 | B | C |
| ATOM | 5572 | CB | CYS H | 313 | −17.840 | 7.078 | −5.071 | 1.00 | 8.77 | B | C |
| ATOM | 5573 | SG | CYS H | 313 | −17.635 | 8.680 | −4.269 | 1.00 | 10.74 | B | S |
| ATOM | 5574 | C | CYS H | 313 | −19.470 | 5.222 | −5.446 | 1.00 | 8.19 | B | C |
| ATOM | 5575 | O | CYS H | 313 | −20.334 | 4.712 | −4.734 | 1.00 | 8.07 | B | O |
| ATOM | 5576 | N | TRP H | 314 | −18.571 | 4.517 | −6.121 | 1.00 | 7.93 | B | N |
| ATOM | 5577 | CA | TRP H | 314 | −18.564 | 3.067 | −6.116 | 1.00 | 7.80 | B | C |
| ATOM | 5578 | CB | TRP H | 314 | −19.648 | 2.528 | −7.050 | 1.00 | 8.13 | B | C |
| ATOM | 5579 | CG | TRP H | 314 | −19.582 | 3.091 | −8.436 | 1.00 | 9.09 | B | C |
| ATOM | 5580 | CD1 | TRP H | 314 | −19.041 | 2.490 | −9.536 | 1.00 | 10.26 | B | C |
| ATOM | 5581 | NE1 | TRP H | 314 | −19.247 | 3.268 | −10.650 | 1.00 | 10.86 | B | N |
| ATOM | 5582 | CE2 | TRP H | 314 | −19.940 | 4.392 | −10.285 | 1.00 | 10.68 | B | C |
| ATOM | 5583 | CD2 | TRP H | 314 | −20.172 | 4.313 | −8.897 | 1.00 | 10.49 | B | C |
| ATOM | 5584 | CE3 | TRP H | 314 | −20.877 | 5.347 | −8.271 | 1.00 | 11.67 | B | C |
| ATOM | 5585 | CZ3 | TRP H | 314 | −21.301 | 6.419 | −9.037 | 1.00 | 12.98 | B | C |
| ATOM | 5586 | CH2 | TRP H | 314 | −21.048 | 6.472 | −10.413 | 1.00 | 12.74 | B | C |
| ATOM | 5587 | CZ2 | TRP H | 314 | −20.369 | 5.472 | −11.054 | 1.00 | 11.73 | B | C |
| ATOM | 5588 | C | TRP H | 314 | −17.198 | 2.576 | −6.563 | 1.00 | 7.34 | B | C |
| ATOM | 5589 | O | TRP H | 314 | −16.485 | 3.272 | −7.288 | 1.00 | 7.47 | B | O |
| ATOM | 5590 | N | LYS H | 315 | −16.791 | 1.426 | −6.041 | 1.00 | 6.75 | B | N |
| ATOM | 5591 | CA | LYS H | 315 | −15.547 | 0.807 | −6.462 | 1.00 | 6.37 | B | C |
| ATOM | 5592 | CB | LYS H | 315 | −14.741 | 0.337 | −5.252 | 1.00 | 6.38 | B | C |
| ATOM | 5593 | CG | LYS H | 315 | −13.277 | 0.071 | −5.554 | 1.00 | 6.55 | B | C |
| ATOM | 5594 | CD | LYS H | 315 | −12.509 | −0.282 | −4.292 | 1.00 | 8.01 | B | C |
| ATOM | 5595 | CE | LYS H | 315 | −11.014 | −0.331 | −4.556 | 1.00 | 8.78 | B | C |
| ATOM | 5596 | NZ | LYS H | 315 | −10.337 | −1.364 | −3.726 | 1.00 | 10.21 | B | N |
| ATOM | 5597 | C | LYS H | 315 | −15.825 | −0.361 | −7.392 | 1.00 | 6.32 | B | C |
| ATOM | 5598 | O | LYS H | 315 | −16.746 | −1.145 | −7.164 | 1.00 | 6.21 | B | O |
| ATOM | 5599 | N | LEU H | 316 | −15.089 | −0.411 | −8.495 | 1.00 | 6.58 | B | N |
| ATOM | 5600 | CA | LEU H | 316 | −15.304 | −1.435 | −9.502 | 1.00 | 7.02 | B | C |
| ATOM | 5601 | CB | LEU H | 316 | −15.491 | −0.801 | −10.880 | 1.00 | 6.64 | B | C |
| ATOM | 5602 | CG | LEU H | 316 | −15.452 | −1.767 | −12.065 | 1.00 | 6.21 | B | C |
| ATOM | 5603 | CD1 | LEU H | 316 | −16.540 | −2.822 | −11.936 | 1.00 | 5.94 | B | C |
| ATOM | 5604 | CD2 | LEU H | 316 | −15.590 | −1.011 | −13.378 | 1.00 | 6.57 | B | C |
| ATOM | 5605 | C | LEU H | 316 | −14.138 | −2.409 | −9.525 | 1.00 | 7.77 | B | C |
| ATOM | 5606 | O | LEU H | 316 | −12.999 | −2.024 | −9.792 | 1.00 | 8.47 | B | O |
| ATOM | 5607 | N | HIS H | 317 | −14.414 | −3.656 | −9.168 | 1.00 | 8.05 | B | N |
| ATOM | 5608 | CA | HIS H | 317 | −13.460 | −4.725 | −9.388 | 1.00 | 8.21 | B | C |
| ATOM | 5609 | CB | HIS H | 317 | −13.266 | −5.547 | −8.116 | 1.00 | 9.04 | B | C |
| ATOM | 5610 | CG | HIS H | 317 | −13.449 | −4.761 | −6.856 | 1.00 | 11.41 | B | C |
| ATOM | 5611 | ND1 | HIS H | 317 | −12.392 | −4.368 | −6.063 | 1.00 | 13.62 | B | N |
| ATOM | 5612 | CE1 | HIS H | 317 | −12.854 | −3.746 | −4.993 | 1.00 | 14.91 | B | C |
| ATOM | 5613 | NE2 | HIS H | 317 | −14.172 | −3.725 | −5.060 | 1.00 | 14.25 | B | N |
| ATOM | 5614 | CD2 | HIS H | 317 | −14.570 | −4.352 | −6.216 | 1.00 | 12.67 | B | C |
| ATOM | 5615 | C | HIS H | 317 | −13.904 | −5.625 | −10.528 | 1.00 | 7.39 | B | C |
| ATOM | 5616 | O | HIS H | 317 | −15.085 | −5.677 | −10.870 | 1.00 | 7.23 | B | O |
| ATOM | 5617 | N | THR H | 318 | −12.976 | −6.451 | −10.993 | 1.00 | 7.11 | B | N |
| ATOM | 5618 | CA | THR H | 318 | −12.973 | −6.913 | −12.372 | 1.00 | 6.56 | B | C |
| ATOM | 5619 | CB | THR H | 318 | −12.242 | −5.919 | −13.295 | 1.00 | 6.28 | B | C |
| ATOM | 5620 | OG1 | THR H | 318 | −13.197 | −5.193 | −14.078 | 1.00 | 6.33 | B | O |
| ATOM | 5621 | CG2 | THR H | 318 | −11.296 | −6.662 | −14.224 | 1.00 | 6.52 | B | C |
| ATOM | 5622 | C | THR H | 318 | −12.254 | −8.251 | −12.438 | 1.00 | 6.58 | B | C |
| ATOM | 5623 | O | THR H | 318 | −11.142 | −8.393 | −11.930 | 1.00 | 6.67 | B | O |
| ATOM | 5624 | N | SER H | 319 | −12.919 | −9.252 | −13.001 | 1.00 | 6.81 | B | N |
| ATOM | 5625 | CA | SER H | 319 | −12.328 | −10.578 | −13.115 | 1.00 | 7.31 | B | C |
| ATOM | 5626 | CB | SER H | 319 | −12.902 | −11.513 | −12.047 | 1.00 | 7.14 | B | C |
| ATOM | 5627 | OG | SER H | 319 | −12.244 | −12.769 | −12.052 | 1.00 | 6.84 | B | O |
| ATOM | 5628 | C | SER H | 319 | −12.533 | −11.169 | −14.509 | 1.00 | 7.93 | B | C |
| ATOM | 5629 | O | SER H | 319 | −13.583 | −10.975 | −15.124 | 1.00 | 8.65 | B | O |
| ATOM | 5630 | N | PRO H | 320 | −11.534 | −11.917 | −15.002 | 1.00 | 8.08 | B | N |
| ATOM | 5631 | CA | PRO H | 320 | −11.592 | −12.459 | −16.354 | 1.00 | 8.34 | B | C |
| ATOM | 5632 | CB | PRO H | 320 | −10.339 | −13.342 | −16.442 | 1.00 | 7.99 | B | C |
| ATOM | 5633 | CG | PRO H | 320 | −9.773 | −13.404 | −15.045 | 1.00 | 8.01 | B | C |
| ATOM | 5634 | CD | PRO H | 320 | −10.230 | −12.163 | −14.367 | 1.00 | 8.10 | B | C |
| ATOM | 5635 | C | PRO H | 320 | −12.848 | −13.288 | −16.610 | 1.00 | 9.03 | B | C |
| ATOM | 5636 | O | PRO H | 320 | −13.372 | −13.928 | −15.697 | 1.00 | 8.74 | B | O |
| ATOM | 5637 | N | LEU H | 321 | −13.224 | −13.384 | −17.881 | 1.00 | 10.31 | B | N |
| ATOM | 5638 | CA | LEU H | 321 | −14.514 | −13.933 | −18.284 | 1.00 | 11.63 | B | C |
| ATOM | 5639 | CB | LEU H | 321 | −15.543 | −12.807 | −18.448 | 1.00 | 11.31 | B | C |
| ATOM | 5640 | CG | LEU H | 321 | −17.032 | −13.163 | −18.454 | 1.00 | 10.97 | B | C |
| ATOM | 5641 | CD1 | LEU H | 321 | −17.828 | −12.071 | −19.154 | 1.00 | 10.04 | B | C |
| ATOM | 5642 | CD2 | LEU H | 321 | −17.273 | −14.514 | −19.113 | 1.00 | 10.69 | B | C |
| ATOM | 5643 | C | LEU H | 321 | −14.359 | −14.687 | −19.603 | 1.00 | 12.92 | B | C |
| ATOM | 5644 | O | LEU H | 321 | −13.871 | −14.135 | −20.591 | 1.00 | 13.29 | B | O |
| ATOM | 5645 | N | CYS H | 322 | −14.788 | −15.947 | −19.611 | 1.00 | 13.39 | B | N |
| ATOM | 5646 | CA | CYS H | 322 | −14.579 | −16.836 | −20.751 | 1.00 | 14.16 | B | C |
| ATOM | 5647 | CB | CYS H | 322 | −13.459 | −17.833 | −20.451 | 1.00 | 13.72 | B | C |
| ATOM | 5648 | SG | CYS H | 322 | −11.996 | −17.091 | −19.704 | 1.00 | 13.42 | B | S |
| ATOM | 5649 | C | CYS H | 322 | −15.859 | −17.589 | −21.115 | 1.00 | 15.46 | B | C |
| ATOM | 5650 | O | CYS H | 322 | −16.561 | −18.099 | −20.241 | 1.00 | 15.94 | B | O |
| ATOM | 5651 | N | THR H | 323 | −16.097 | −17.735 | −22.415 | 1.00 | 16.70 | B | N |
| ATOM | 5652 | CA | THR H | 323 | −17.059 | −18.703 | −22.940 | 1.00 | 17.84 | B | C |
| ATOM | 5653 | CB | THR H | 323 | −17.054 | −18.690 | −24.477 | 1.00 | 17.50 | B | C |
| ATOM | 5654 | OG1 | THR H | 323 | −15.767 | −18.268 | −24.947 | 1.00 | 15.70 | B | O |
| ATOM | 5655 | CG2 | THR H | 323 | −18.118 | −17.740 | −25.005 | 1.00 | 18.05 | B | C |
| ATOM | 5656 | C | THR H | 323 | −16.755 | −20.125 | −22.469 | 1.00 | 19.54 | B | C |
| ATOM | 5657 | O | THR H | 323 | −15.670 | −20.389 | −21.950 | 1.00 | 19.84 | B | O |
| ATOM | 5658 | N | THR H | 324 | −17.625 | −21.068 | −22.825 | 1.00 | 21.17 | B | N |
| ATOM | 5659 | CA | THR H | 324 | −17.251 | −22.481 | −22.774 | 1.00 | 22.89 | B | C |
| ATOM | 5660 | CB | THR H | 324 | −17.589 | −23.138 | −21.426 | 1.00 | 22.69 | B | C |
| ATOM | 5661 | OG1 | THR H | 324 | −18.025 | −22.141 | −20.494 | 1.00 | 22.10 | B | O |
| ATOM | 5662 | CG2 | THR H | 324 | −16.365 | −23.854 | −20.871 | 1.00 | 22.03 | B | C |
| ATOM | 5663 | C | THR H | 324 | −17.737 | −23.375 | −23.915 | 1.00 | 24.39 | B | C |
| ATOM | 5664 | O | THR H | 324 | −17.251 | −24.495 | −24.070 | 1.00 | 24.53 | B | O |
| ATOM | 5665 | N | ASN H | 325 | −18.633 | −22.866 | −24.755 | 1.00 | 25.72 | B | N |
| ATOM | 5666 | CA | ASN H | 325 | −18.864 | −23.475 | −26.064 | 1.00 | 27.01 | B | C |
| ATOM | 5667 | CB | ASN H | 325 | −19.924 | −24.578 | −25.977 | 1.00 | 27.37 | B | C |
| ATOM | 5668 | CG | ASN H | 325 | −19.337 | −25.925 | −25.593 | 1.00 | 28.46 | B | C |
| ATOM | 5669 | OD1 | ASN H | 325 | −19.134 | −26.792 | −26.443 | 1.00 | 30.37 | B | O |
| ATOM | 5670 | ND2 | ASN H | 325 | −19.086 | −26.114 | −24.303 | 1.00 | 28.78 | B | N |
| ATOM | 5671 | C | ASN H | 325 | −19.246 | −22.460 | −27.136 | 1.00 | 27.36 | B | C |
| ATOM | 5672 | O | ASN H | 325 | −19.634 | −21.334 | −26.829 | 1.00 | 27.94 | B | O |
| ATOM | 5673 | N | SER H | 330 | −15.365 | −21.167 | −24.863 | 1.00 | 35.76 | B | N |
| ATOM | 5674 | CA | SER H | 330 | −14.200 | −21.669 | −25.580 | 1.00 | 35.01 | B | C |
| ATOM | 5675 | CB | SER H | 330 | −14.630 | −22.631 | −26.690 | 1.00 | 35.47 | B | C |
| ATOM | 5676 | OG | SER H | 330 | −15.521 | −22.000 | −27.595 | 1.00 | 33.31 | B | O |
| ATOM | 5677 | C | SER H | 330 | −13.383 | −20.522 | −26.168 | 1.00 | 34.35 | B | C |
| ATOM | 5678 | O | SER H | 330 | −13.938 | −19.509 | −26.593 | 1.00 | 34.33 | B | O |
| ATOM | 5679 | N | ASN H | 331 | −12.062 | −20.653 | −26.123 | 1.00 | 32.48 | B | N |
| ATOM | 5680 | CA | ASN H | 331 | −11.191 | −19.933 | −27.046 | 1.00 | 30.72 | B | C |
| ATOM | 5681 | CB | ASN H | 331 | −11.577 | −20.233 | −28.504 | 1.00 | 31.31 | B | C |
| ATOM | 5682 | CG | ASN H | 331 | −11.276 | −21.670 | −28.918 | 1.00 | 32.11 | B | C |
| ATOM | 5683 | OD1 | ASN H | 331 | −10.553 | −22.391 | −28.229 | 1.00 | 32.54 | B | O |
| ATOM | 5684 | ND2 | ASN H | 331 | −11.753 | −22.055 | −30.097 | 1.00 | 31.30 | B | N |
| ATOM | 5685 | C | ASN H | 331 | −11.120 | −18.411 | −26.826 | 1.00 | 28.71 | B | C |
| ATOM | 5686 | O | ASN H | 331 | −10.495 | −17.710 | −27.621 | 1.00 | 29.07 | B | O |
| ATOM | 5687 | N | ILE H | 332 | −11.797 | −17.887 | −25.802 | 1.00 | 25.44 | B | N |
| ATOM | 5688 | CA | ILE H | 332 | −11.904 | −16.424 | −25.638 | 1.00 | 21.83 | B | C |
| ATOM | 5689 | CB | ILE H | 332 | −12.919 | −15.806 | −26.624 | 1.00 | 21.42 | B | C |
| ATOM | 5690 | CG1 | ILE H | 332 | −12.916 | −14.280 | −26.511 | 1.00 | 19.89 | B | C |
| ATOM | 5691 | CD1 | ILE H | 332 | −11.986 | −13.596 | −27.488 | 1.00 | 17.67 | B | C |
| ATOM | 5692 | CG2 | ILE H | 332 | −14.314 | −16.360 | −26.374 | 1.00 | 20.61 | B | C |
| ATOM | 5693 | C | ILE H | 332 | −12.204 | −15.918 | −24.217 | 1.00 | 20.37 | B | C |
| ATOM | 5694 | O | ILE H | 332 | −13.219 | −16.290 | −23.624 | 1.00 | 19.95 | B | O |
| ATOM | 5695 | N | CYS H | 333 | −11.425 | −14.935 | −23.761 | 1.00 | 18.27 | B | N |
| ATOM | 5696 | CA | CYS H | 333 | −11.681 | −14.282 | −22.474 | 1.00 | 16.47 | B | C |
| ATOM | 5697 | CB | CYS H | 333 | −10.802 | −14.873 | −21.371 | 1.00 | 16.14 | B | C |
| ATOM | 5698 | SG | CYS H | 333 | −10.807 | −16.672 | −21.295 | 1.00 | 17.09 | B | S |
| ATOM | 5699 | C | CYS H | 333 | −11.542 | −12.763 | −22.482 | 1.00 | 15.73 | B | C |
| ATOM | 5700 | O | CYS H | 333 | −10.707 | −12.206 | −23.197 | 1.00 | 15.43 | B | O |
| ATOM | 5701 | N | LEU H | 334 | −12.154 | −12.155 | −21.470 | 1.00 | 15.06 | B | N |
| ATOM | 5702 | CA | LEU H | 334 | −12.256 | −10.708 | −21.353 | 1.00 | 13.79 | B | C |
| ATOM | 5703 | CB | LEU H | 334 | −13.702 | −10.264 | −21.588 | 1.00 | 13.39 | B | C |
| ATOM | 5704 | CG | LEU H | 334 | −14.262 | −10.341 | −23.008 | 1.00 | 13.93 | B | C |
| ATOM | 5705 | CD1 | LEU H | 334 | −15.276 | −9.230 | −23.235 | 1.00 | 14.20 | B | C |
| ATOM | 5706 | CD2 | LEU H | 334 | −13.143 | −10.266 | −24.035 | 1.00 | 14.52 | B | C |
| ATOM | 5707 | C | LEU H | 334 | −11.868 | −10.345 | −19.930 | 1.00 | 13.22 | B | C |
| ATOM | 5708 | O | LEU H | 334 | −12.324 | −10.980 | −18.980 | 1.00 | 13.31 | B | O |
| ATOM | 5709 | N | THR H | 335 | −11.141 | −9.246 | −19.776 | 1.00 | 12.58 | B | N |
| ATOM | 5710 | CA | THR H | 335 | −11.033 | −8.596 | −18.478 | 1.00 | 12.17 | B | C |
| ATOM | 5711 | CB | THR H | 335 | −9.677 | −8.904 | −17.798 | 1.00 | 12.02 | B | C |
| ATOM | 5712 | OG1 | THR H | 335 | −9.207 | −10.191 | −18.219 | 1.00 | 12.64 | B | O |
| ATOM | 5713 | CG2 | THR H | 335 | −9.817 | −8.893 | −16.280 | 1.00 | 11.47 | B | C |
| ATOM | 5714 | C | THR H | 335 | −11.203 | −7.092 | −18.639 | 1.00 | 11.97 | B | C |
| ATOM | 5715 | O | THR H | 335 | −10.637 | −6.493 | −19.553 | 1.00 | 11.93 | B | O |
| ATOM | 5716 | N | ARG H | 336 | −12.130 | −6.526 | −17.874 | 1.00 | 11.60 | B | N |
| ATOM | 5717 | CA | ARG H | 336 | −12.411 | −5.098 | −17.949 | 1.00 | 12.15 | B | C |
| ATOM | 5718 | CB | ARG H | 336 | −13.716 | −4.767 | −17.215 | 1.00 | 12.16 | B | C |
| ATOM | 5719 | CG | ARG H | 336 | −14.525 | −3.643 | −17.849 | 1.00 | 12.67 | B | C |
| ATOM | 5720 | CD | ARG H | 336 | −15.993 | −4.018 | −17.970 | 1.00 | 12.52 | B | C |
| ATOM | 5721 | NE | ARG H | 336 | −16.736 | −3.718 | −16.750 | 1.00 | 12.35 | B | N |
| ATOM | 5722 | CZ | ARG H | 336 | −17.058 | −2.490 | −16.355 | 1.00 | 12.54 | B | C |
| ATOM | 5723 | NH1 | ARG H | 336 | −16.665 | −1.439 | −17.062 | 1.00 | 12.38 | B | N |
| ATOM | 5724 | NH2 | ARG H | 336 | −17.761 | −2.312 | −15.246 | 1.00 | 12.48 | B | N |
| ATOM | 5725 | C | ARG H | 336 | −11.252 | −4.295 | −17.361 | 1.00 | 12.71 | B | C |
| ATOM | 5726 | O | ARG H | 336 | −10.891 | −4.475 | −16.198 | 1.00 | 12.62 | B | O |
| ATOM | 5727 | N | THR H | 337 | −10.615 | −3.475 | −18.192 | 1.00 | 13.70 | B | N |
| ATOM | 5728 | CA | THR H | 337 | −9.431 | −2.733 | −17.766 | 1.00 | 14.91 | B | C |
| ATOM | 5729 | CB | THR H | 337 | −8.736 | −2.032 | −18.949 | 1.00 | 15.27 | B | C |
| ATOM | 5730 | OG1 | THR H | 337 | −8.620 | −2.946 | −20.046 | 1.00 | 16.65 | B | O |
| ATOM | 5731 | CG2 | THR H | 337 | −7.347 | −1.560 | −18.546 | 1.00 | 15.94 | B | C |
| ATOM | 5732 | C | THR H | 337 | −9.759 | −1.707 | −16.681 | 1.00 | 15.16 | B | C |
| ATOM | 5733 | O | THR H | 337 | −9.100 | −1.659 | −15.643 | 1.00 | 15.60 | B | O |
| ATOM | 5734 | N | ASP H | 338 | −10.770 | −0.880 | −16.934 | 1.00 | 15.15 | B | N |
| ATOM | 5735 | CA | ASP H | 338 | −10.988 | 0.325 | −16.143 | 1.00 | 15.52 | B | C |
| ATOM | 5736 | CB | ASP H | 338 | −11.999 | 1.250 | −16.828 | 1.00 | 16.81 | B | C |
| ATOM | 5737 | CG | ASP H | 338 | −13.028 | 0.489 | −17.642 | 1.00 | 20.57 | B | C |
| ATOM | 5738 | OD1 | ASP H | 338 | −13.670 | 1.103 | −18.519 | 1.00 | 23.00 | B | O |
| ATOM | 5739 | OD2 | ASP H | 338 | −13.224 | −0.716 | −17.378 | 1.00 | 22.92 | B | O |
| ATOM | 5740 | C | ASP H | 338 | −11.453 | −0.022 | −14.733 | 1.00 | 14.21 | B | C |
| ATOM | 5741 | O | ASP H | 338 | −12.651 | −0.171 | −14.482 | 1.00 | 14.00 | B | O |
| ATOM | 5742 | N | ARG H | 339 | −10.494 | −0.189 | −13.827 | 1.00 | 12.79 | B | N |
| ATOM | 5743 | CA | ARG H | 339 | −10.792 | −0.528 | −12.440 | 1.00 | 11.71 | B | C |
| ATOM | 5744 | CB | ARG H | 339 | −9.866 | −1.630 | −11.950 | 1.00 | 11.73 | B | C |
| ATOM | 5745 | CG | ARG H | 339 | −9.941 | −2.905 | −12.727 | 1.00 | 12.49 | B | C |
| ATOM | 5746 | CD | ARG H | 339 | −9.329 | −3.991 | −11.895 | 1.00 | 13.24 | B | C |
| ATOM | 5747 | NE | ARG H | 339 | −8.677 | −5.006 | −12.705 | 1.00 | 12.04 | B | N |
| ATOM | 5748 | CZ | ARG H | 339 | −8.332 | −6.200 | −12.239 | 1.00 | 11.74 | B | C |
| ATOM | 5749 | NH1 | ARG H | 339 | −8.540 | −6.498 | −10.964 | 1.00 | 13.43 | B | N |
| ATOM | 5750 | NH2 | ARG H | 339 | −7.784 | −7.097 | −13.045 | 1.00 | 9.80 | B | N |
| ATOM | 5751 | C | ARG H | 339 | −10.626 | 0.666 | −11.519 | 1.00 | 11.16 | B | C |
| ATOM | 5752 | O | ARG H | 339 | −9.690 | 1.448 | −11.681 | 1.00 | 11.37 | B | O |
| ATOM | 5753 | N | GLY H | 340 | −11.290 | 0.573 | −10.373 | 1.00 | 10.43 | B | N |
| ATOM | 5754 | CA | GLY H | 340 | −11.038 | 1.481 | −9.269 | 1.00 | 9.52 | B | C |
| ATOM | 5755 | C | GLY H | 340 | −12.279 | 2.268 | −8.916 | 1.00 | 8.80 | B | C |
| ATOM | 5756 | O | GLY H | 340 | −13.397 | 1.759 | −8.996 | 1.00 | 8.56 | B | O |
| ATOM | 5757 | N | TRP H | 341 | −12.077 | 3.513 | −8.506 | 1.00 | 8.25 | B | N |
| ATOM | 5758 | CA | TRP H | 341 | −13.156 | 4.309 | −7.953 | 1.00 | 7.98 | B | C |
| ATOM | 5759 | CB | TRP H | 341 | −12.629 | 5.235 | −6.865 | 1.00 | 7.79 | B | C |
| ATOM | 5760 | CG | TRP H | 341 | −12.435 | 4.538 | −5.579 | 1.00 | 7.67 | B | C |
| ATOM | 5761 | CD1 | TRP H | 341 | −11.275 | 3.990 | −5.119 | 1.00 | 8.12 | B | C |
| ATOM | 5762 | NE1 | TRP H | 341 | −11.496 | 3.346 | −3.928 | 1.00 | 8.60 | B | N |
| ATOM | 5763 | CE2 | TRP H | 341 | −12.834 | 3.401 | −3.639 | 1.00 | 7.57 | B | C |
| ATOM | 5764 | CD2 | TRP H | 341 | −13.464 | 4.093 | −4.691 | 1.00 | 7.52 | B | C |
| ATOM | 5765 | CE3 | TRP H | 341 | −14.848 | 4.291 | −4.636 | 1.00 | 7.86 | B | C |
| ATOM | 5766 | CZ3 | TRP H | 341 | −15.545 | 3.797 | −3.548 | 1.00 | 7.14 | B | C |
| ATOM | 5767 | CH2 | TRP H | 341 | −14.890 | 3.105 | −2.522 | 1.00 | 6.39 | B | C |
| ATOM | 5768 | CZ2 | TRP H | 341 | −13.538 | 2.903 | −2.546 | 1.00 | 7.30 | B | C |
| ATOM | 5769 | C | TRP H | 341 | −13.835 | 5.117 | −9.037 | 1.00 | 8.26 | B | C |
| ATOM | 5770 | O | TRP H | 341 | −13.222 | 5.978 | −9.666 | 1.00 | 8.35 | B | O |
| ATOM | 5771 | N | TYR H | 342 | −15.113 | 4.840 | −9.244 | 1.00 | 8.44 | B | N |
| ATOM | 5772 | CA | TYR H | 342 | −16.000 | 5.809 | −9.847 | 1.00 | 8.53 | B | C |
| ATOM | 5773 | CB | TYR H | 342 | −16.948 | 5.116 | −10.814 | 1.00 | 8.43 | B | C |
| ATOM | 5774 | CG | TYR H | 342 | −16.214 | 4.338 | −11.870 | 1.00 | 9.17 | B | C |
| ATOM | 5775 | CD1 | TYR H | 342 | −15.551 | 3.163 | −11.540 | 1.00 | 10.32 | B | C |
| ATOM | 5776 | CE1 | TYR H | 342 | −14.766 | 2.503 | −12.465 | 1.00 | 10.99 | B | C |
| ATOM | 5777 | CZ | TYR H | 342 | −14.566 | 3.063 | −13.709 | 1.00 | 11.00 | B | C |
| ATOM | 5778 | OH | TYR H | 342 | −13.828 | 2.387 | −14.653 | 1.00 | 12.82 | B | O |
| ATOM | 5779 | CE2 | TYR H | 342 | −15.115 | 4.288 | −14.019 | 1.00 | 10.67 | B | C |
| ATOM | 5780 | CD2 | TYR H | 342 | −15.913 | 4.930 | −13.091 | 1.00 | 10.41 | B | C |
| ATOM | 5781 | C | TYR H | 342 | −16.781 | 6.535 | −8.778 | 1.00 | 8.64 | B | C |
| ATOM | 5782 | O | TYR H | 342 | −16.830 | 6.103 | −7.628 | 1.00 | 8.40 | B | O |
| ATOM | 5783 | N | CYS H | 343 | −17.373 | 7.657 | −9.157 | 1.00 | 8.87 | B | N |
| ATOM | 5784 | CA | CYS H | 343 | −17.812 | 8.625 | −8.178 | 1.00 | 9.35 | B | C |
| ATOM | 5785 | CB | CYS H | 343 | −16.702 | 8.901 | −7.160 | 1.00 | 9.17 | B | C |
| ATOM | 5786 | SG | CYS H | 343 | −17.204 | 9.963 | −5.787 | 1.00 | 11.88 | B | S |
| ATOM | 5787 | C | CYS H | 343 | −18.258 | 9.907 | −8.863 | 1.00 | 9.42 | B | C |
| ATOM | 5788 | O | CYS H | 343 | −17.552 | 10.444 | −9.717 | 1.00 | 9.96 | B | O |
| ATOM | 5789 | N | ASP H | 344 | −19.523 | 10.250 | −8.643 | 1.00 | 9.36 | B | N |
| ATOM | 5790 | CA | ASP H | 344 | −20.231 | 11.213 | −9.477 | 1.00 | 9.24 | B | C |
| ATOM | 5791 | CB | ASP H | 344 | −21.712 | 11.250 | −9.097 | 1.00 | 9.34 | B | C |
| ATOM | 5792 | CG | ASP H | 344 | −22.555 | 10.320 | −9.943 | 1.00 | 10.72 | B | C |
| ATOM | 5793 | OD1 | ASP H | 344 | −22.582 | 10.500 | −11.178 | 1.00 | 13.93 | B | O |
| ATOM | 5794 | OD2 | ASP H | 344 | −23.246 | 9.453 | −9.367 | 1.00 | 11.87 | B | O |
| ATOM | 5795 | C | ASP H | 344 | −19.632 | 12.607 | −9.330 | 1.00 | 9.09 | B | C |
| ATOM | 5796 | O | ASP H | 344 | −19.454 | 13.101 | −8.216 | 1.00 | 9.39 | B | O |
| ATOM | 5797 | N | ASN H | 345 | −19.416 | 13.274 | −10.457 | 1.00 | 9.13 | B | N |
| ATOM | 5798 | CA | ASN H | 345 | −19.165 | 14.709 | −10.461 | 1.00 | 9.52 | B | C |
| ATOM | 5799 | CB | ASN H | 345 | −17.833 | 15.011 | −11.149 | 1.00 | 9.61 | B | C |
| ATOM | 5800 | CG | ASN H | 345 | −17.166 | 16.258 | −10.606 | 1.00 | 9.21 | B | C |
| ATOM | 5801 | OD1 | ASN H | 345 | −16.787 | 16.316 | −9.437 | 1.00 | 6.21 | B | O |
| ATOM | 5802 | ND2 | ASN H | 345 | −17.023 | 17.268 | −11.456 | 1.00 | 11.05 | B | N |
| ATOM | 5803 | C | ASN H | 345 | −20.303 | 15.452 | −11.152 | 1.00 | 10.06 | B | C |
| ATOM | 5804 | O | ASN H | 345 | −21.347 | 14.867 | −11.441 | 1.00 | 11.10 | B | O |
| ATOM | 5805 | N | ALA H | 346 | −20.133 | 16.755 | −11.352 | 1.00 | 9.74 | B | N |
| ATOM | 5806 | CA | ALA H | 346 | −21.211 | 17.584 | −11.877 | 1.00 | 9.58 | B | C |
| ATOM | 5807 | CB | ALA H | 346 | −20.708 | 18.991 | −12.164 | 1.00 | 9.60 | B | C |
| ATOM | 5808 | C | ALA H | 346 | −21.825 | 16.968 | −13.133 | 1.00 | 9.66 | B | C |
| ATOM | 5809 | O | ALA H | 346 | −21.347 | 17.196 | −14.244 | 1.00 | 9.39 | B | O |
| ATOM | 5810 | N | GLY H | 347 | −22.883 | 16.182 | −12.948 | 1.00 | 10.04 | B | N |
| ATOM | 5811 | CA | GLY H | 347 | −23.684 | 15.689 | −14.067 | 1.00 | 10.68 | B | C |
| ATOM | 5812 | C | GLY H | 347 | −22.964 | 14.606 | −14.846 | 1.00 | 10.90 | B | C |
| ATOM | 5813 | O | GLY H | 347 | −23.490 | 14.064 | −15.819 | 1.00 | 11.46 | B | O |
| ATOM | 5814 | N | SER H | 348 | −21.714 | 14.370 | −14.467 | 1.00 | 10.30 | B | N |
| ATOM | 5815 | CA | SER H | 348 | −20.844 | 13.436 | −15.165 | 1.00 | 9.94 | B | C |
| ATOM | 5816 | CB | SER H | 348 | −19.833 | 14.200 | −16.022 | 1.00 | 9.88 | B | C |
| ATOM | 5817 | OG | SER H | 348 | −20.488 | 15.064 | −16.934 | 1.00 | 10.09 | B | O |
| ATOM | 5818 | C | SER H | 348 | −20.120 | 12.619 | −14.104 | 1.00 | 9.60 | B | C |
| ATOM | 5819 | O | SER H | 348 | −20.367 | 12.796 | −12.912 | 1.00 | 9.59 | B | O |
| ATOM | 5820 | N | VAL H | 349 | −19.286 | 11.677 | −14.528 | 1.00 | 9.54 | B | N |
| ATOM | 5821 | CA | VAL H | 349 | −18.647 | 10.766 | −13.581 | 1.00 | 9.56 | B | C |
| ATOM | 5822 | CB | VAL H | 349 | −19.075 | 9.305 | −13.809 | 1.00 | 9.28 | B | C |
| ATOM | 5823 | CG1 | VAL H | 349 | −18.659 | 8.442 | −12.626 | 1.00 | 9.24 | B | C |
| ATOM | 5824 | CG2 | VAL H | 349 | −20.576 | 9.222 | −14.033 | 1.00 | 9.94 | B | C |
| ATOM | 5825 | C | VAL H | 349 | −17.126 | 10.861 | −13.619 | 1.00 | 9.59 | B | C |
| ATOM | 5826 | O | VAL H | 349 | −16.513 | 10.752 | −14.681 | 1.00 | 9.69 | B | O |
| ATOM | 5827 | N | SER H | 350 | −16.520 | 11.027 | −12.449 | 1.00 | 9.93 | B | N |
| ATOM | 5828 | CA | SER H | 350 | −15.073 | 11.104 | −12.357 | 1.00 | 10.44 | B | C |
| ATOM | 5829 | CB | SER H | 350 | −14.646 | 12.227 | −11.408 | 1.00 | 10.04 | B | C |
| ATOM | 5830 | OG | SER H | 350 | −14.584 | 13.468 | −12.090 | 1.00 | 8.41 | B | O |
| ATOM | 5831 | C | SER H | 350 | −14.459 | 9.773 | −11.938 | 1.00 | 11.45 | B | C |
| ATOM | 5832 | O | SER H | 350 | −15.000 | 9.062 | −11.089 | 1.00 | 11.58 | B | O |
| ATOM | 5833 | N | PHE H | 351 | −13.354 | 9.424 | −12.587 | 1.00 | 12.28 | B | N |
| ATOM | 5834 | CA | PHE H | 351 | −12.717 | 8.129 | −12.403 | 1.00 | 13.04 | B | C |
| ATOM | 5835 | CB | PHE H | 351 | −12.800 | 7.315 | −13.697 | 1.00 | 12.87 | B | C |
| ATOM | 5836 | CG | PHE H | 351 | −11.985 | 6.052 | −13.677 | 1.00 | 13.52 | B | C |
| ATOM | 5837 | CD1 | PHE H | 351 | −12.081 | 5.165 | −12.615 | 1.00 | 13.76 | B | C |
| ATOM | 5838 | CE1 | PHE H | 351 | −11.300 | 4.022 | −12.574 | 1.00 | 14.10 | B | C |
| ATOM | 5839 | CZ | PHE H | 351 | −10.453 | 3.726 | −13.629 | 1.00 | 14.07 | B | C |
| ATOM | 5840 | CE2 | PHE H | 351 | −10.381 | 4.583 | −14.715 | 1.00 | 14.14 | B | C |
| ATOM | 5841 | CD2 | PHE H | 351 | −11.134 | 5.744 | −14.728 | 1.00 | 13.92 | B | C |
| ATOM | 5842 | C | PHE H | 351 | −11.262 | 8.315 | −11.993 | 1.00 | 13.84 | B | C |
| ATOM | 5843 | O | PHE H | 351 | −10.528 | 9.090 | −12.608 | 1.00 | 13.96 | B | O |
| ATOM | 5844 | N | PHE H | 352 | −10.866 | 7.641 | −10.919 | 1.00 | 14.72 | B | N |
| ATOM | 5845 | CA | PHE H | 352 | −9.500 | 7.717 | −10.418 | 1.00 | 15.98 | B | C |
| ATOM | 5846 | CB | PHE H | 352 | −9.490 | 8.210 | −8.966 | 1.00 | 15.75 | B | C |
| ATOM | 5847 | CG | PHE H | 352 | −10.600 | 9.176 | −8.638 | 1.00 | 15.51 | B | C |
| ATOM | 5848 | CD1 | PHE H | 352 | −11.835 | 8.713 | −8.210 | 1.00 | 15.05 | B | C |
| ATOM | 5849 | CE1 | PHE H | 352 | −12.836 | 9.597 | −7.837 | 1.00 | 14.05 | B | C |
| ATOM | 5850 | CZ | PHE H | 352 | −12.592 | 10.955 | −7.844 | 1.00 | 13.28 | B | C |
| ATOM | 5851 | CE2 | PHE H | 352 | −11.343 | 11.426 | −8.201 | 1.00 | 13.71 | B | C |
| ATOM | 5852 | CD2 | PHE H | 352 | −10.347 | 10.536 | −8.570 | 1.00 | 14.94 | B | C |
| ATOM | 5853 | C | PHE H | 352 | −8.820 | 6.350 | −10.511 | 1.00 | 17.56 | B | C |
| ATOM | 5854 | O | PHE H | 352 | −9.259 | 5.390 | −9.880 | 1.00 | 17.74 | B | O |
| ATOM | 5855 | N | PRO H | 353 | −7.728 | 6.266 | −11.288 | 1.00 | 19.05 | B | N |
| ATOM | 5856 | CA | PRO H | 353 | −7.203 | 5.002 | −11.802 | 1.00 | 20.40 | B | C |
| ATOM | 5857 | CB | PRO H | 353 | −6.180 | 5.443 | −12.859 | 1.00 | 20.26 | B | C |
| ATOM | 5858 | CG | PRO H | 353 | −6.492 | 6.878 | −13.145 | 1.00 | 19.71 | B | C |
| ATOM | 5859 | CD | PRO H | 353 | −7.032 | 7.424 | −11.869 | 1.00 | 18.97 | B | C |
| ATOM | 5860 | C | PRO H | 353 | −6.522 | 4.135 | −10.743 | 1.00 | 21.94 | B | C |
| ATOM | 5861 | O | PRO H | 353 | −6.511 | 2.911 | −10.873 | 1.00 | 22.29 | B | O |
| ATOM | 5862 | N | GLN H | 354 | −5.904 | 4.757 | −9.742 | 1.00 | 23.38 | B | N |
| ATOM | 5863 | CA | GLN H | 354 | −5.118 | 4.007 | −8.760 | 1.00 | 24.64 | B | C |
| ATOM | 5864 | CB | GLN H | 354 | −3.621 | 4.108 | −9.064 | 1.00 | 24.93 | B | C |
| ATOM | 5865 | CG | GLN H | 354 | −3.259 | 5.210 | −10.038 | 1.00 | 26.00 | B | C |
| ATOM | 5866 | CD | GLN H | 354 | −3.331 | 6.581 | −9.407 | 1.00 | 25.92 | B | C |
| ATOM | 5867 | OE1 | GLN H | 354 | −2.732 | 6.830 | −8.360 | 1.00 | 25.67 | B | O |
| ATOM | 5868 | NE2 | GLN H | 354 | −4.094 | 7.475 | −10.025 | 1.00 | 24.27 | B | N |
| ATOM | 5869 | C | GLN H | 354 | −5.393 | 4.393 | −7.307 | 1.00 | 24.85 | B | C |
| ATOM | 5870 | O | GLN H | 354 | −5.653 | 5.558 | −7.007 | 1.00 | 24.51 | B | O |
| ATOM | 5871 | N | ALA H | 355 | −5.162 | 3.446 | −6.399 | 1.00 | 25.22 | B | N |
| ATOM | 5872 | CA | ALA H | 355 | −5.563 | 3.599 | −4.997 | 1.00 | 25.34 | B | C |
| ATOM | 5873 | CB | ALA H | 355 | −5.112 | 2.400 | −4.173 | 1.00 | 25.09 | B | C |
| ATOM | 5874 | C | ALA H | 355 | −5.092 | 4.910 | −4.354 | 1.00 | 24.92 | B | C |
| ATOM | 5875 | O | ALA H | 355 | −5.901 | 5.647 | −3.790 | 1.00 | 25.23 | B | O |
| ATOM | 5876 | N | GLU H | 356 | −3.775 | 5.074 | −4.242 | 1.00 | 23.93 | B | N |
| ATOM | 5877 | CA | GLU H | 356 | −3.138 | 6.394 | −4.141 | 1.00 | 22.77 | B | C |
| ATOM | 5878 | CB | GLU H | 356 | −2.214 | 6.635 | −5.339 | 1.00 | 23.22 | B | C |
| ATOM | 5879 | CG | GLU H | 356 | −0.973 | 5.756 | −5.344 | 1.00 | 26.33 | B | C |
| ATOM | 5880 | CD | GLU H | 356 | −0.571 | 5.312 | −3.948 | 1.00 | 30.93 | B | C |
| ATOM | 5881 | OE1 | GLU H | 356 | −1.012 | 4.225 | −3.516 | 1.00 | 31.97 | B | O |
| ATOM | 5882 | OE2 | GLU H | 356 | 0.074 | 6.111 | −3.237 | 1.00 | 32.21 | B | O |
| ATOM | 5883 | C | GLU H | 356 | −4.114 | 7.558 | −3.990 | 1.00 | 21.31 | B | C |
| ATOM | 5884 | O | GLU H | 356 | −4.098 | 8.270 | −2.985 | 1.00 | 21.01 | B | O |
| ATOM | 5885 | N | THR H | 357 | −4.868 | 7.819 | −5.052 | 1.00 | 19.51 | B | N |
| ATOM | 5886 | CA | THR H | 357 | −5.718 | 9.000 | −5.138 | 1.00 | 17.55 | B | C |
| ATOM | 5887 | CB | THR H | 357 | −6.391 | 9.086 | −6.519 | 1.00 | 17.30 | B | C |
| ATOM | 5888 | OG1 | THR H | 357 | −5.644 | 8.305 | −7.462 | 1.00 | 17.32 | B | O |
| ATOM | 5889 | CG2 | THR H | 357 | −6.472 | 10.531 | −6.997 | 1.00 | 17.72 | B | C |
| ATOM | 5890 | C | THR H | 357 | −6.801 | 9.006 | −4.063 | 1.00 | 16.62 | B | C |
| ATOM | 5891 | O | THR H | 357 | −7.346 | 10.058 | −3.730 | 1.00 | 17.29 | B | O |
| ATOM | 5892 | N | CYS H | 358 | −7.179 | 7.822 | −3.594 | 1.00 | 14.88 | B | N |
| ATOM | 5893 | CA | CYS H | 358 | −8.414 | 7.676 | −2.833 | 1.00 | 13.15 | B | C |
| ATOM | 5894 | CB | CYS H | 358 | −9.479 | 6.949 | −3.655 | 1.00 | 13.04 | B | C |
| ATOM | 5895 | SG | CYS H | 358 | −10.003 | 7.836 | −5.144 | 1.00 | 14.02 | B | S |
| ATOM | 5896 | C | CYS H | 358 | −8.202 | 6.982 | −1.492 | 1.00 | 12.08 | B | C |
| ATOM | 5897 | O | CYS H | 358 | −7.521 | 5.959 | −1.408 | 1.00 | 12.15 | B | O |
| ATOM | 5898 | N | LYS H | 359 | −8.741 | 7.585 | −0.438 | 1.00 | 11.35 | B | N |
| ATOM | 5899 | CA | LYS H | 359 | −8.861 | 6.922 | 0.853 | 1.00 | 11.01 | B | C |
| ATOM | 5900 | CB | LYS H | 359 | −8.119 | 7.713 | 1.931 | 1.00 | 11.16 | B | C |
| ATOM | 5901 | CG | LYS H | 359 | −7.413 | 8.953 | 1.414 | 1.00 | 13.28 | B | C |
| ATOM | 5902 | CD | LYS H | 359 | −5.907 | 8.816 | 1.540 | 1.00 | 16.11 | B | C |
| ATOM | 5903 | CE | LYS H | 359 | −5.199 | 10.088 | 1.104 | 1.00 | 17.91 | B | C |
| ATOM | 5904 | NZ | LYS H | 359 | −3.719 | 9.952 | 1.184 | 1.00 | 20.99 | B | N |
| ATOM | 5905 | C | LYS H | 359 | −10.325 | 6.777 | 1.242 | 1.00 | 10.46 | B | C |
| ATOM | 5906 | O | LYS H | 359 | −11.191 | 7.462 | 0.697 | 1.00 | 10.70 | B | O |
| ATOM | 5907 | N | VAL H | 360 | −10.579 | 5.977 | 2.270 | 1.00 | 9.99 | B | N |
| ATOM | 5908 | CA | VAL H | 360 | −11.939 | 5.576 | 2.592 | 1.00 | 9.84 | B | C |
| ATOM | 5909 | CB | VAL H | 360 | −12.406 | 4.401 | 1.712 | 1.00 | 9.42 | B | C |
| ATOM | 5910 | CG1 | VAL H | 360 | −11.411 | 3.253 | 1.784 | 1.00 | 10.07 | B | C |
| ATOM | 5911 | CG2 | VAL H | 360 | −13.796 | 3.944 | 2.125 | 1.00 | 9.56 | B | C |
| ATOM | 5912 | C | VAL H | 360 | −12.087 | 5.214 | 4.063 | 1.00 | 9.98 | B | C |
| ATOM | 5913 | O | VAL H | 360 | −11.267 | 4.485 | 4.622 | 1.00 | 9.96 | B | O |
| ATOM | 5914 | N | GLN H | 361 | −13.109 | 5.780 | 4.696 | 1.00 | 10.39 | B | N |
| ATOM | 5915 | CA | GLN H | 361 | −13.322 | 5.606 | 6.125 | 1.00 | 11.53 | B | C |
| ATOM | 5916 | CB | GLN H | 361 | −12.858 | 6.851 | 6.883 | 1.00 | 12.03 | B | C |
| ATOM | 5917 | CG | GLN H | 361 | −13.051 | 6.769 | 8.385 | 1.00 | 16.44 | B | C |
| ATOM | 5918 | CD | GLN H | 361 | −11.812 | 7.176 | 9.152 | 1.00 | 25.16 | B | C |
| ATOM | 5919 | OE1 | GLN H | 361 | −11.763 | 8.257 | 9.738 | 1.00 | 29.01 | B | O |
| ATOM | 5920 | NE2 | GLN H | 361 | −10.773 | 6.352 | 9.080 | 1.00 | 27.20 | B | N |
| ATOM | 5921 | C | GLN H | 361 | −14.795 | 5.340 | 6.415 | 1.00 | 11.15 | B | C |
| ATOM | 5922 | O | GLN H | 361 | −15.620 | 6.253 | 6.355 | 1.00 | 11.80 | B | O |
| ATOM | 5923 | N | SER H | 362 | −15.136 | 4.078 | 6.650 | 1.00 | 10.90 | B | N |
| ATOM | 5924 | CA | SER H | 362 | −16.529 | 3.677 | 6.634 | 1.00 | 10.30 | B | C |
| ATOM | 5925 | CB | SER H | 362 | −17.326 | 4.548 | 7.603 | 1.00 | 10.47 | B | C |
| ATOM | 5926 | OG | SER H | 362 | −18.616 | 4.011 | 7.823 | 1.00 | 11.17 | B | O |
| ATOM | 5927 | C | SER H | 362 | −17.074 | 3.846 | 5.224 | 1.00 | 9.58 | B | C |
| ATOM | 5928 | O | SER H | 362 | −16.419 | 3.474 | 4.250 | 1.00 | 9.48 | B | O |
| ATOM | 5929 | N | ASN H | 363 | −18.182 | 4.569 | 5.115 | 1.00 | 9.12 | B | N |
| ATOM | 5930 | CA | ASN H | 363 | −18.810 | 4.826 | 3.826 | 1.00 | 8.73 | B | C |
| ATOM | 5931 | CB | ASN H | 363 | −20.329 | 4.778 | 3.972 | 1.00 | 9.01 | B | C |
| ATOM | 5932 | CG | ASN H | 363 | −20.850 | 5.801 | 4.965 | 1.00 | 9.29 | B | C |
| ATOM | 5933 | OD1 | ASN H | 363 | −20.079 | 6.555 | 5.561 | 1.00 | 9.82 | B | O |
| ATOM | 5934 | ND2 | ASN H | 363 | −22.162 | 5.818 | 5.163 | 1.00 | 10.08 | B | N |
| ATOM | 5935 | C | ASN H | 363 | −18.382 | 6.185 | 3.277 | 1.00 | 8.12 | B | C |
| ATOM | 5936 | O | ASN H | 363 | −18.996 | 6.720 | 2.352 | 1.00 | 8.10 | B | O |
| ATOM | 5937 | N | ARG H | 364 | −17.346 | 6.749 | 3.890 | 1.00 | 7.20 | B | N |
| ATOM | 5938 | CA | ARG H | 364 | −16.777 | 8.017 | 3.457 | 1.00 | 6.82 | B | C |
| ATOM | 5939 | CB | ARG H | 364 | −16.307 | 8.821 | 4.671 | 1.00 | 6.91 | B | C |
| ATOM | 5940 | CG | ARG H | 364 | −16.741 | 10.271 | 4.655 | 1.00 | 7.92 | B | C |
| ATOM | 5941 | CD | ARG H | 364 | −18.197 | 10.397 | 4.249 | 1.00 | 8.20 | B | C |
| ATOM | 5942 | NE | ARG H | 364 | −18.377 | 11.314 | 3.127 | 1.00 | 8.70 | B | N |
| ATOM | 5943 | CZ | ARG H | 364 | −19.450 | 12.077 | 2.958 | 1.00 | 8.88 | B | C |
| ATOM | 5944 | NH1 | ARG H | 364 | −20.420 | 12.062 | 3.862 | 1.00 | 8.57 | B | N |
| ATOM | 5945 | NH2 | ARG H | 364 | −19.546 | 12.867 | 1.897 | 1.00 | 10.59 | B | N |
| ATOM | 5946 | C | ARG H | 364 | −15.603 | 7.775 | 2.519 | 1.00 | 6.58 | B | C |
| ATOM | 5947 | O | ARG H | 364 | −14.680 | 7.030 | 2.847 | 1.00 | 6.65 | B | O |
| ATOM | 5948 | N | VAL H | 365 | −15.619 | 8.448 | 1.375 | 1.00 | 6.64 | B | N |
| ATOM | 5949 | CA | VAL H | 365 | −14.526 | 8.350 | 0.419 | 1.00 | 6.54 | B | C |
| ATOM | 5950 | CB | VAL H | 365 | −15.000 | 7.763 | −0.922 | 1.00 | 5.95 | B | C |
| ATOM | 5951 | CG1 | VAL H | 365 | −13.934 | 7.950 | −1.990 | 1.00 | 5.48 | B | C |
| ATOM | 5952 | CG2 | VAL H | 365 | −15.350 | 6.292 | −0.762 | 1.00 | 6.22 | B | C |
| ATOM | 5953 | C | VAL H | 365 | −13.914 | 9.719 | 0.166 | 1.00 | 7.35 | B | C |
| ATOM | 5954 | O | VAL H | 365 | −14.617 | 10.729 | 0.128 | 1.00 | 8.33 | B | O |
| ATOM | 5955 | N | PHE H | 366 | −12.611 | 9.733 | −0.081 | 1.00 | 7.37 | B | N |
| ATOM | 5956 | CA | PHE H | 366 | −11.904 | 10.969 | −0.360 | 1.00 | 7.44 | B | C |
| ATOM | 5957 | CB | PHE H | 366 | −11.137 | 11.431 | 0.874 | 1.00 | 7.30 | B | C |
| ATOM | 5958 | CG | PHE H | 366 | −12.009 | 11.714 | 2.054 | 1.00 | 6.70 | B | C |
| ATOM | 5959 | CD1 | PHE H | 366 | −12.571 | 10.677 | 2.778 | 1.00 | 6.70 | B | C |
| ATOM | 5960 | CE1 | PHE H | 366 | −13.347 | 10.932 | 3.894 | 1.00 | 5.94 | B | C |
| ATOM | 5961 | CZ | PHE H | 366 | −13.546 | 12.236 | 4.311 | 1.00 | 5.71 | B | C |
| ATOM | 5962 | CE2 | PHE H | 366 | −12.990 | 13.279 | 3.593 | 1.00 | 5.96 | B | C |
| ATOM | 5963 | CD2 | PHE H | 366 | −12.235 | 13.015 | 2.467 | 1.00 | 6.52 | B | C |
| ATOM | 5964 | C | PHE H | 366 | −10.939 | 10.777 | −1.511 | 1.00 | 7.45 | B | C |
| ATOM | 5965 | O | PHE H | 366 | −9.931 | 10.082 | −1.381 | 1.00 | 7.45 | B | O |
| ATOM | 5966 | N | CYS H | 367 | −11.212 | 11.453 | −2.618 | 1.00 | 8.00 | B | N |
| ATOM | 5967 | CA | CYS H | 367 | −10.408 | 11.281 | −3.809 | 1.00 | 8.90 | B | C |
| ATOM | 5968 | CB | CYS H | 367 | −11.217 | 10.596 | −4.909 | 1.00 | 9.46 | B | C |
| ATOM | 5969 | SG | CYS H | 367 | −11.640 | 8.877 | −4.545 | 1.00 | 10.79 | B | S |
| ATOM | 5970 | C | CYS H | 367 | −9.870 | 12.612 | −4.297 | 1.00 | 9.09 | B | C |
| ATOM | 5971 | O | CYS H | 367 | −10.363 | 13.674 | −3.919 | 1.00 | 9.24 | B | O |
| ATOM | 5972 | N | ASP H | 368 | −8.892 | 12.540 | −5.188 | 1.00 | 9.54 | B | N |
| ATOM | 5973 | CA | ASP H | 368 | −8.190 | 13.724 | −5.637 | 1.00 | 10.55 | B | C |
| ATOM | 5974 | CB | ASP H | 368 | −6.693 | 13.584 | −5.372 | 1.00 | 10.53 | B | C |
| ATOM | 5975 | CG | ASP H | 368 | −5.983 | 14.917 | −5.352 | 1.00 | 11.11 | B | C |
| ATOM | 5976 | OD1 | ASP H | 368 | −6.335 | 15.787 | −6.177 | 1.00 | 11.42 | B | O |
| ATOM | 5977 | OD2 | ASP H | 368 | −5.158 | 15.138 | −4.441 | 1.00 | 11.35 | B | O |
| ATOM | 5978 | C | ASP H | 368 | −8.442 | 13.949 | −7.118 | 1.00 | 11.45 | B | C |
| ATOM | 5979 | O | ASP H | 368 | −8.036 | 13.143 | −7.955 | 1.00 | 12.05 | B | O |
| ATOM | 5980 | N | THR H | 369 | −9.222 | 14.980 | −7.425 | 1.00 | 12.41 | B | N |
| ATOM | 5981 | CA | THR H | 369 | −9.359 | 15.444 | −8.796 | 1.00 | 13.00 | B | C |
| ATOM | 5982 | CB | THR H | 369 | −9.723 | 16.934 | −8.846 | 1.00 | 12.69 | B | C |
| ATOM | 5983 | OG1 | THR H | 369 | −10.937 | 17.158 | −8.117 | 1.00 | 11.72 | B | O |
| ATOM | 5984 | CG2 | THR H | 369 | −9.896 | 17.390 | −10.289 | 1.00 | 13.33 | B | C |
| ATOM | 5985 | C | THR H | 369 | −8.052 | 15.254 | −9.548 | 1.00 | 14.13 | B | C |
| ATOM | 5986 | O | THR H | 369 | −7.986 | 14.504 | −10.520 | 1.00 | 14.72 | B | O |
| ATOM | 5987 | N | MET H | 370 | −6.996 | 15.868 | −9.029 | 1.00 | 15.25 | B | N |
| ATOM | 5988 | CA | MET H | 370 | −5.822 | 16.195 | −9.825 | 1.00 | 16.60 | B | C |
| ATOM | 5989 | CB | MET H | 370 | −4.542 | 15.983 | −9.019 | 1.00 | 17.42 | B | C |
| ATOM | 5990 | CG | MET H | 370 | −3.815 | 17.270 | −8.673 | 1.00 | 20.31 | B | C |
| ATOM | 5991 | SD | MET H | 370 | −3.256 | 18.144 | −10.144 | 1.00 | 26.43 | B | S |
| ATOM | 5992 | CE | MET H | 370 | −4.817 | 18.449 | −10.970 | 1.00 | 24.07 | B | C |
| ATOM | 5993 | C | MET H | 370 | −5.752 | 15.466 | −11.165 | 1.00 | 16.54 | B | C |
| ATOM | 5994 | O | MET H | 370 | −5.659 | 16.108 | −12.209 | 1.00 | 16.74 | B | O |
| ATOM | 5995 | N | ASN H | 371 | −5.730 | 14.137 | −11.146 | 1.00 | 16.35 | B | N |
| ATOM | 5996 | CA | ASN H | 371 | −5.564 | 13.390 | −12.394 | 1.00 | 16.07 | B | C |
| ATOM | 5997 | CB | ASN H | 371 | −4.271 | 12.573 | −12.386 | 1.00 | 16.41 | B | C |
| ATOM | 5998 | CG | ASN H | 371 | −3.047 | 13.428 | −12.644 | 1.00 | 16.71 | B | C |
| ATOM | 5999 | OD1 | ASN H | 371 | −2.747 | 13.766 | −13.790 | 1.00 | 15.75 | B | O |
| ATOM | 6000 | ND2 | ASN H | 371 | −2.401 | 13.874 | −11.572 | 1.00 | 17.76 | B | N |
| ATOM | 6001 | C | ASN H | 371 | −6.767 | 12.552 | −12.827 | 1.00 | 15.55 | B | C |
| ATOM | 6002 | O | ASN H | 371 | −6.757 | 11.959 | −13.907 | 1.00 | 15.84 | B | O |
| ATOM | 6003 | N | SER H | 372 | −7.879 | 12.750 | −12.126 | 1.00 | 14.76 | B | N |
| ATOM | 6004 | CA | SER H | 372 | −9.165 | 12.189 | −12.526 | 1.00 | 13.91 | B | C |
| ATOM | 6005 | CB | SER H | 372 | −10.309 | 12.932 | −11.832 | 1.00 | 14.04 | B | C |
| ATOM | 6006 | OG | SER H | 372 | −10.226 | 14.328 | −12.061 | 1.00 | 15.09 | B | O |
| ATOM | 6007 | C | SER H | 372 | −9.372 | 12.202 | −14.037 | 1.00 | 13.07 | B | C |
| ATOM | 6008 | O | SER H | 372 | −8.839 | 13.056 | −14.743 | 1.00 | 13.02 | B | O |
| ATOM | 6009 | N | LEU H | 373 | −10.112 | 11.213 | −14.526 | 1.00 | 12.23 | B | N |
| ATOM | 6010 | CA | LEU H | 373 | −10.687 | 11.256 | −15.864 | 1.00 | 11.45 | B | C |
| ATOM | 6011 | CB | LEU H | 373 | −10.417 | 9.939 | −16.594 | 1.00 | 11.43 | B | C |
| ATOM | 6012 | CG | LEU H | 373 | −9.204 | 9.880 | −17.524 | 1.00 | 11.04 | B | C |
| ATOM | 6013 | CD1 | LEU H | 373 | −7.988 | 10.551 | −16.888 | 1.00 | 11.76 | B | C |
| ATOM | 6014 | CD2 | LEU H | 373 | −8.894 | 8.435 | −17.915 | 1.00 | 10.49 | B | C |
| ATOM | 6015 | C | LEU H | 373 | −12.188 | 11.472 | −15.762 | 1.00 | 11.17 | B | C |
| ATOM | 6016 | O | LEU H | 373 | −12.847 | 10.886 | −14.904 | 1.00 | 11.27 | B | O |
| ATOM | 6017 | N | THR H | 374 | −12.733 | 12.289 | −16.655 | 1.00 | 10.70 | B | N |
| ATOM | 6018 | CA | THR H | 374 | −14.144 | 12.637 | −16.586 | 1.00 | 10.23 | B | C |
| ATOM | 6019 | CB | THR H | 374 | −14.367 | 14.151 | −16.730 | 1.00 | 10.24 | B | C |
| ATOM | 6020 | OG1 | THR H | 374 | −13.177 | 14.850 | −16.344 | 1.00 | 11.25 | B | O |
| ATOM | 6021 | CG2 | THR H | 374 | −15.521 | 14.604 | −15.849 | 1.00 | 10.52 | B | C |
| ATOM | 6022 | C | THR H | 374 | −14.953 | 11.896 | −17.641 | 1.00 | 9.54 | B | C |
| ATOM | 6023 | O | THR H | 374 | −14.580 | 11.855 | −18.813 | 1.00 | 9.70 | B | O |
| ATOM | 6024 | N | LEU H | 375 | −16.035 | 11.266 | −17.198 | 1.00 | 8.83 | B | N |
| ATOM | 6025 | CA | LEU H | 375 | −16.731 | 10.270 | −18.000 | 1.00 | 7.83 | B | C |
| ATOM | 6026 | CB | LEU H | 375 | −16.360 | 8.861 | −17.540 | 1.00 | 7.38 | B | C |
| ATOM | 6027 | CG | LEU H | 375 | −14.878 | 8.495 | −17.551 | 1.00 | 5.06 | B | C |
| ATOM | 6028 | CD1 | LEU H | 375 | −14.710 | 7.045 | −17.124 | 1.00 | 2.41 | B | C |
| ATOM | 6029 | CD2 | LEU H | 375 | −14.288 | 8.729 | −18.934 | 1.00 | 4.31 | B | C |
| ATOM | 6030 | C | LEU H | 375 | −18.236 | 10.456 | −17.878 | 1.00 | 7.79 | B | C |
| ATOM | 6031 | O | LEU H | 375 | −18.724 | 10.928 | −16.850 | 1.00 | 7.92 | B | O |
| ATOM | 6032 | N | PRO H | 376 | −18.982 | 9.954 | −18.871 | 1.00 | 7.51 | B | N |
| ATOM | 6033 | CA | PRO H | 376 | −20.439 | 10.009 | −18.882 | 1.00 | 7.90 | B | C |
| ATOM | 6034 | CB | PRO H | 376 | −20.780 | 9.837 | −20.362 | 1.00 | 7.90 | B | C |
| ATOM | 6035 | CG | PRO H | 376 | −19.657 | 9.033 | −20.908 | 1.00 | 7.97 | B | C |
| ATOM | 6036 | CD | PRO H | 376 | −18.435 | 9.468 | −20.150 | 1.00 | 7.40 | B | C |
| ATOM | 6037 | C | PRO H | 376 | −21.063 | 8.881 | −18.064 | 1.00 | 8.35 | B | C |
| ATOM | 6038 | O | PRO H | 376 | −20.569 | 7.753 | −18.085 | 1.00 | 8.71 | B | O |
| ATOM | 6039 | N | SER H | 377 | −22.190 | 9.172 | −17.421 | 1.00 | 9.11 | B | N |
| ATOM | 6040 | CA | SER H | 377 | −22.937 | 8.165 | −16.673 | 1.00 | 9.91 | B | C |
| ATOM | 6041 | CB | SER H | 377 | −24.272 | 8.737 | −16.191 | 1.00 | 10.23 | B | C |
| ATOM | 6042 | OG | SER H | 377 | −24.293 | 10.150 | −16.304 | 1.00 | 10.90 | B | O |
| ATOM | 6043 | C | SER H | 377 | −23.180 | 6.915 | −17.513 | 1.00 | 10.14 | B | C |
| ATOM | 6044 | O | SER H | 377 | −23.678 | 5.906 | −17.013 | 1.00 | 10.08 | B | O |
| ATOM | 6045 | N | GLU H | 378 | −22.875 | 7.010 | −18.803 | 1.00 | 10.71 | B | N |
| ATOM | 6046 | CA | GLU H | 378 | −23.057 | 5.893 | −19.721 | 1.00 | 11.40 | B | C |
| ATOM | 6047 | CB | GLU H | 378 | −22.970 | 6.377 | −21.170 | 1.00 | 11.65 | B | C |
| ATOM | 6048 | CG | GLU H | 378 | −24.261 | 6.977 | −21.706 | 1.00 | 13.46 | B | C |
| ATOM | 6049 | CD | GLU H | 378 | −24.671 | 8.241 | −20.972 | 1.00 | 16.20 | B | C |
| ATOM | 6050 | OE1 | GLU H | 378 | −23.922 | 8.681 | −20.074 | 1.00 | 15.73 | B | O |
| ATOM | 6051 | OE2 | GLU H | 378 | −25.749 | 8.788 | −21.285 | 1.00 | 18.38 | B | O |
| ATOM | 6052 | C | GLU H | 378 | −22.019 | 4.805 | −19.468 | 1.00 | 11.27 | B | C |
| ATOM | 6053 | O | GLU H | 378 | −22.196 | 3.657 | −19.877 | 1.00 | 11.28 | B | O |
| ATOM | 6054 | N | VAL H | 379 | −20.943 | 5.170 | −18.779 | 1.00 | 11.31 | B | N |
| ATOM | 6055 | CA | VAL H | 379 | −19.977 | 4.192 | −18.296 | 1.00 | 11.59 | B | C |
| ATOM | 6056 | CB | VAL H | 379 | −18.973 | 4.824 | −17.315 | 1.00 | 11.27 | B | C |
| ATOM | 6057 | CG1 | VAL H | 379 | −18.343 | 3.752 | −16.440 | 1.00 | 11.60 | B | C |
| ATOM | 6058 | CG2 | VAL H | 379 | −17.905 | 5.597 | −18.073 | 1.00 | 11.54 | B | C |
| ATOM | 6059 | C | VAL H | 379 | −20.675 | 3.023 | −17.610 | 1.00 | 12.16 | B | C |
| ATOM | 6060 | O | VAL H | 379 | −20.260 | 1.872 | −17.750 | 1.00 | 12.51 | B | O |
| ATOM | 6061 | N | ASN H | 380 | −21.769 | 3.317 | −16.915 | 1.00 | 12.98 | B | N |
| ATOM | 6062 | CA | ASN H | 380 | −22.428 | 2.328 | −16.068 | 1.00 | 14.00 | B | C |
| ATOM | 6063 | CB | ASN H | 380 | −23.553 | 2.973 | −15.258 | 1.00 | 14.59 | B | C |
| ATOM | 6064 | CG | ASN H | 380 | −23.038 | 3.964 | −14.234 | 1.00 | 17.09 | B | C |
| ATOM | 6065 | OD1 | ASN H | 380 | −22.893 | 3.637 | −13.056 | 1.00 | 20.41 | B | O |
| ATOM | 6066 | ND2 | ASN H | 380 | −22.690 | 5.160 | −14.693 | 1.00 | 18.86 | B | N |
| ATOM | 6067 | C | ASN H | 380 | −22.954 | 1.115 | −16.833 | 1.00 | 13.96 | B | C |
| ATOM | 6068 | O | ASN H | 380 | −22.973 | 0.005 | −16.302 | 1.00 | 14.38 | B | O |
| ATOM | 6069 | N | LEU H | 381 | −23.459 | 1.343 | −18.042 | 1.00 | 13.61 | B | N |
| ATOM | 6070 | CA | LEU H | 381 | −24.024 | 0.261 | −18.844 | 1.00 | 13.72 | B | C |
| ATOM | 6071 | CB | LEU H | 381 | −24.514 | 0.792 | −20.191 | 1.00 | 13.79 | B | C |
| ATOM | 6072 | CG | LEU H | 381 | −25.004 | 2.240 | −20.194 | 1.00 | 14.92 | B | C |
| ATOM | 6073 | CD1 | LEU H | 381 | −25.409 | 2.670 | −21.595 | 1.00 | 15.56 | B | C |
| ATOM | 6074 | CD2 | LEU H | 381 | −26.157 | 2.422 | −19.218 | 1.00 | 15.78 | B | C |
| ATOM | 6075 | C | LEU H | 381 | −22.982 | −0.827 | −19.060 | 1.00 | 13.62 | B | C |
| ATOM | 6076 | O | LEU H | 381 | −23.314 | −1.973 | −19.365 | 1.00 | 13.57 | B | O |
| ATOM | 6077 | N | CYS H | 382 | −21.729 | −0.478 | −18.796 | 1.00 | 13.47 | B | N |
| ATOM | 6078 | CA | CYS H | 382 | −20.615 | −1.367 | −19.068 | 1.00 | 13.57 | B | C |
| ATOM | 6079 | CB | CYS H | 382 | −19.321 | −0.565 | −19.165 | 1.00 | 13.62 | B | C |
| ATOM | 6080 | SG | CYS H | 382 | −18.814 | −0.217 | −20.852 | 1.00 | 15.64 | B | S |
| ATOM | 6081 | C | CYS H | 382 | −20.494 | −2.432 | −17.987 | 1.00 | 13.33 | B | C |
| ATOM | 6082 | O | CYS H | 382 | −19.948 | −3.510 | −18.224 | 1.00 | 13.57 | B | O |
| ATOM | 6083 | N | ASN H | 383 | −21.096 | −2.163 | −16.833 | 1.00 | 13.21 | B | N |
| ATOM | 6084 | CA | ASN H | 383 | −21.267 | −3.181 | −15.803 | 1.00 | 13.22 | B | C |
| ATOM | 6085 | CB | ASN H | 383 | −21.854 | −2.568 | −14.531 | 1.00 | 13.18 | B | C |
| ATOM | 6086 | CG | ASN H | 383 | −21.059 | −1.381 | −14.034 | 1.00 | 13.42 | B | C |
| ATOM | 6087 | OD1 | ASN H | 383 | −19.830 | −1.418 | −13.985 | 1.00 | 13.68 | B | O |
| ATOM | 6088 | ND2 | ASN H | 383 | −21.761 | −0.330 | −13.626 | 1.00 | 14.32 | B | N |
| ATOM | 6089 | C | ASN H | 383 | −22.159 | −4.317 | −16.277 | 1.00 | 13.28 | B | C |
| ATOM | 6090 | O | ASN H | 383 | −22.096 | −5.428 | −15.751 | 1.00 | 13.50 | B | O |
| ATOM | 6091 | N | VAL H | 384 | −23.092 | −3.990 | −17.164 | 1.00 | 13.24 | B | N |
| ATOM | 6092 | CA | VAL H | 384 | −24.065 | −4.964 | −17.630 | 1.00 | 13.29 | B | C |
| ATOM | 6093 | CB | VAL H | 384 | −25.484 | −4.367 | −17.709 | 1.00 | 13.28 | B | C |
| ATOM | 6094 | CG1 | VAL H | 384 | −26.371 | −5.222 | −18.601 | 1.00 | 13.73 | B | C |
| ATOM | 6095 | CG2 | VAL H | 384 | −26.083 | −4.241 | −16.315 | 1.00 | 12.76 | B | C |
| ATOM | 6096 | C | VAL H | 384 | −23.677 | −5.563 | −18.977 | 1.00 | 13.57 | B | C |
| ATOM | 6097 | O | VAL H | 384 | −23.940 | −6.737 | −19.233 | 1.00 | 13.31 | B | O |
| ATOM | 6098 | N | ASP H | 385 | −23.060 | −4.764 | −19.843 | 1.00 | 14.07 | B | N |
| ATOM | 6099 | CA | ASP H | 385 | −22.923 | −5.172 | −21.238 | 1.00 | 14.61 | B | C |
| ATOM | 6100 | CB | ASP H | 385 | −23.915 | −4.459 | −22.154 | 1.00 | 14.71 | B | C |
| ATOM | 6101 | CG | ASP H | 385 | −24.584 | −5.410 | −23.130 | 1.00 | 16.69 | B | C |
| ATOM | 6102 | OD1 | ASP H | 385 | −24.093 | −6.549 | −23.281 | 1.00 | 18.63 | B | O |
| ATOM | 6103 | OD2 | ASP H | 385 | −25.614 | −5.030 | −23.724 | 1.00 | 19.10 | B | O |
| ATOM | 6104 | C | ASP H | 385 | −21.525 | −5.244 | −21.851 | 1.00 | 14.63 | B | C |
| ATOM | 6105 | O | ASP H | 385 | −21.203 | −6.221 | −22.526 | 1.00 | 14.34 | B | O |
| ATOM | 6106 | N | ILE H | 386 | −20.722 | −4.198 | −21.691 | 1.00 | 14.54 | B | N |
| ATOM | 6107 | CA | ILE H | 386 | −19.435 | −4.146 | −22.382 | 1.00 | 14.46 | B | C |
| ATOM | 6108 | CB | ILE H | 386 | −18.772 | −5.540 | −22.417 | 1.00 | 13.83 | B | C |
| ATOM | 6109 | CG1 | ILE H | 386 | −18.450 | −6.032 | −21.009 | 1.00 | 13.44 | B | C |
| ATOM | 6110 | CD1 | ILE H | 386 | −18.701 | −7.516 | −20.821 | 1.00 | 12.39 | B | C |
| ATOM | 6111 | CG2 | ILE H | 386 | −17.533 | −5.525 | −23.298 | 1.00 | 13.51 | B | C |
| ATOM | 6112 | C | ILE H | 386 | −19.673 | −3.755 | −23.830 | 1.00 | 14.73 | B | C |
| ATOM | 6113 | O | ILE H | 386 | −19.266 | −2.682 | −24.274 | 1.00 | 14.68 | B | O |
| ATOM | 6114 | N | PHE H | 387 | −20.135 | −4.733 | −24.601 | 1.00 | 14.71 | B | N |
| ATOM | 6115 | CA | PHE H | 387 | −20.696 | −4.482 | −25.915 | 1.00 | 14.78 | B | C |
| ATOM | 6116 | CB | PHE H | 387 | −20.739 | −5.766 | −26.733 | 1.00 | 14.77 | B | C |
| ATOM | 6117 | CG | PHE H | 387 | −19.482 | −6.569 | −26.647 | 1.00 | 14.75 | B | C |
| ATOM | 6118 | CD1 | PHE H | 387 | −19.407 | −7.658 | −25.797 | 1.00 | 15.03 | B | C |
| ATOM | 6119 | CE1 | PHE H | 387 | −18.229 | −8.368 | −25.662 | 1.00 | 14.62 | B | C |
| ATOM | 6120 | CZ | PHE H | 387 | −17.093 | −7.943 | −26.318 | 1.00 | 14.55 | B | C |
| ATOM | 6121 | CE2 | PHE H | 387 | −17.136 | −6.809 | −27.103 | 1.00 | 14.68 | B | C |
| ATOM | 6122 | CD2 | PHE H | 387 | −18.316 | −6.103 | −27.227 | 1.00 | 14.61 | B | C |
| ATOM | 6123 | C | PHE H | 387 | −22.080 | −3.871 | −25.821 | 1.00 | 14.91 | B | C |
| ATOM | 6124 | O | PHE H | 387 | −22.992 | −4.445 | −25.228 | 1.00 | 14.86 | B | O |
| ATOM | 6125 | N | ASN H | 388 | −22.144 | −2.610 | −26.216 | 1.00 | 15.11 | B | N |
| ATOM | 6126 | CA | ASN H | 388 | −23.383 | −1.869 | −26.295 | 1.00 | 15.38 | B | C |
| ATOM | 6127 | CB | ASN H | 388 | −23.968 | −1.663 | −24.897 | 1.00 | 14.86 | B | C |
| ATOM | 6128 | CG | ASN H | 388 | −23.005 | −0.958 | −23.963 | 1.00 | 14.86 | B | C |
| ATOM | 6129 | OD1 | ASN H | 388 | −22.936 | 0.270 | −23.935 | 1.00 | 12.80 | B | O |
| ATOM | 6130 | ND2 | ASN H | 388 | −22.213 | −1.736 | −23.234 | 1.00 | 15.97 | B | N |
| ATOM | 6131 | C | ASN H | 388 | −23.023 | −0.526 | −26.906 | 1.00 | 16.11 | B | C |
| ATOM | 6132 | O | ASN H | 388 | −21.965 | 0.024 | −26.599 | 1.00 | 15.92 | B | O |
| ATOM | 6133 | N | PRO H | 389 | −23.772 | −0.117 | −27.935 | 1.00 | 16.99 | B | N |
| ATOM | 6134 | CA | PRO H | 389 | −23.716 | 1.289 | −28.309 | 1.00 | 17.40 | B | C |
| ATOM | 6135 | CB | PRO H | 389 | −24.965 | 1.468 | −29.176 | 1.00 | 17.72 | B | C |
| ATOM | 6136 | CG | PRO H | 389 | −25.904 | 0.425 | −28.694 | 1.00 | 17.47 | B | C |
| ATOM | 6137 | CD | PRO H | 389 | −25.041 | −0.747 | −28.339 | 1.00 | 17.05 | B | C |
| ATOM | 6138 | C | PRO H | 389 | −23.810 | 2.170 | −27.071 | 1.00 | 17.86 | B | C |
| ATOM | 6139 | O | PRO H | 389 | −24.276 | 1.718 | −26.025 | 1.00 | 18.45 | B | O |
| ATOM | 6140 | N | LYS H | 390 | −23.335 | 3.406 | −27.180 | 1.00 | 17.97 | B | N |
| ATOM | 6141 | CA | LYS H | 390 | −23.325 | 4.318 | −26.045 | 1.00 | 18.06 | B | C |
| ATOM | 6142 | CB | LYS H | 390 | −24.373 | 3.904 | −25.009 | 1.00 | 17.92 | B | C |
| ATOM | 6143 | CG | LYS H | 390 | −25.807 | 4.197 | −25.424 | 1.00 | 18.58 | B | C |
| ATOM | 6144 | CD | LYS H | 390 | −26.259 | 5.564 | −24.935 | 1.00 | 20.61 | B | C |
| ATOM | 6145 | CE | LYS H | 390 | −27.775 | 5.656 | −24.875 | 1.00 | 21.48 | B | C |
| ATOM | 6146 | NZ | LYS H | 390 | −28.387 | 5.669 | −26.232 | 1.00 | 23.16 | B | N |
| ATOM | 6147 | C | LYS H | 390 | −21.946 | 4.403 | −25.400 | 1.00 | 18.01 | B | C |
| ATOM | 6148 | O | LYS H | 390 | −21.382 | 5.490 | −25.279 | 1.00 | 18.43 | B | O |
| ATOM | 6149 | N | TYR H | 391 | −21.391 | 3.261 | −25.011 | 1.00 | 17.68 | B | N |
| ATOM | 6150 | CA | TYR H | 391 | −20.012 | 3.232 | −24.539 | 1.00 | 17.33 | B | C |
| ATOM | 6151 | CB | TYR H | 391 | −19.948 | 3.140 | −23.015 | 1.00 | 17.42 | B | C |
| ATOM | 6152 | CG | TYR H | 391 | −18.847 | 3.986 | −22.423 | 1.00 | 17.27 | B | C |
| ATOM | 6153 | CD1 | TYR H | 391 | −18.602 | 5.263 | −22.911 | 1.00 | 17.29 | B | C |
| ATOM | 6154 | CE1 | TYR H | 391 | −17.553 | 6.021 | −22.434 | 1.00 | 17.32 | B | C |
| ATOM | 6155 | CZ | TYR H | 391 | −16.698 | 5.486 | −21.496 | 1.00 | 16.51 | B | C |
| ATOM | 6156 | OH | TYR H | 391 | −15.654 | 6.241 | −21.015 | 1.00 | 16.65 | B | O |
| ATOM | 6157 | CE2 | TYR H | 391 | −16.878 | 4.195 | −21.045 | 1.00 | 16.52 | B | C |
| ATOM | 6158 | CD2 | TYR H | 391 | −17.933 | 3.444 | −21.530 | 1.00 | 17.24 | B | C |
| ATOM | 6159 | C | TYR H | 391 | −19.179 | 2.130 | −25.176 | 1.00 | 17.43 | B | C |
| ATOM | 6160 | O | TYR H | 391 | −19.459 | 0.946 | −24.993 | 1.00 | 17.70 | B | O |
| ATOM | 6161 | N | ASP H | 392 | −18.075 | 2.522 | −25.804 | 1.00 | 17.56 | B | N |
| ATOM | 6162 | CA | ASP H | 392 | −16.973 | 1.599 | −26.012 | 1.00 | 17.69 | B | C |
| ATOM | 6163 | CB | ASP H | 392 | −16.119 | 2.003 | −27.214 | 1.00 | 17.89 | B | C |
| ATOM | 6164 | CG | ASP H | 392 | −16.587 | 3.290 | −27.853 | 1.00 | 18.86 | B | C |
| ATOM | 6165 | OD1 | ASP H | 392 | −16.582 | 4.332 | −27.165 | 1.00 | 20.34 | B | O |
| ATOM | 6166 | OD2 | ASP H | 392 | −16.931 | 3.269 | −29.053 | 1.00 | 18.51 | B | O |
| ATOM | 6167 | C | ASP H | 392 | −16.115 | 1.473 | −24.767 | 1.00 | 17.11 | B | C |
| ATOM | 6168 | O | ASP H | 392 | −15.801 | 2.460 | −24.101 | 1.00 | 17.04 | B | O |
| ATOM | 6169 | N | CYS H | 393 | −15.736 | 0.239 | −24.468 | 1.00 | 16.33 | B | N |
| ATOM | 6170 | CA | CYS H | 393 | −15.683 | −0.240 | −23.102 | 1.00 | 16.17 | B | C |
| ATOM | 6171 | CB | CYS H | 393 | −16.818 | −1.230 | −22.851 | 1.00 | 16.11 | B | C |
| ATOM | 6172 | SG | CYS H | 393 | −17.226 | −1.449 | −21.117 | 1.00 | 20.23 | B | S |
| ATOM | 6173 | C | CYS H | 393 | −14.355 | −0.941 | −22.926 | 1.00 | 15.37 | B | C |
| ATOM | 6174 | O | CYS H | 393 | −14.044 | −1.876 | −23.663 | 1.00 | 15.15 | B | O |
| ATOM | 6175 | N | LYS H | 394 | −13.482 | −0.332 | −22.138 | 1.00 | 14.91 | B | N |
| ATOM | 6176 | CA | LYS H | 394 | −12.100 | −0.755 | −22.107 | 1.00 | 14.50 | B | C |
| ATOM | 6177 | CB | LYS H | 394 | −11.242 | 0.294 | −21.405 | 1.00 | 14.60 | B | C |
| ATOM | 6178 | CG | LYS H | 394 | −11.613 | 1.723 | −21.788 | 1.00 | 16.44 | B | C |
| ATOM | 6179 | CD | LYS H | 394 | −10.460 | 2.688 | −21.576 | 1.00 | 20.45 | B | C |
| ATOM | 6180 | CE | LYS H | 394 | −10.252 | 2.974 | −20.099 | 1.00 | 23.05 | B | C |
| ATOM | 6181 | NZ | LYS H | 394 | −9.330 | 1.988 | −19.467 | 1.00 | 24.13 | B | N |
| ATOM | 6182 | C | LYS H | 394 | −11.955 | −2.130 | −21.463 | 1.00 | 14.00 | B | C |
| ATOM | 6183 | O | LYS H | 394 | −12.593 | −2.434 | −20.454 | 1.00 | 14.14 | B | O |
| ATOM | 6184 | N | ILE H | 395 | −11.118 | −2.957 | −22.077 | 1.00 | 13.28 | B | N |
| ATOM | 6185 | CA | ILE H | 395 | −11.238 | −4.407 | −22.026 | 1.00 | 12.89 | B | C |
| ATOM | 6186 | CB | ILE H | 395 | −12.208 | −4.925 | −23.106 | 1.00 | 12.75 | B | C |
| ATOM | 6187 | CG1 | ILE H | 395 | −13.660 | −4.699 | −22.690 | 1.00 | 13.58 | B | C |
| ATOM | 6188 | CD1 | ILE H | 395 | −14.633 | −4.719 | −23.849 | 1.00 | 15.33 | B | C |
| ATOM | 6189 | CG2 | ILE H | 395 | −11.947 | −6.396 | −23.399 | 1.00 | 12.16 | B | C |
| ATOM | 6190 | C | ILE H | 395 | −9.860 | −4.906 | −22.407 | 1.00 | 13.15 | B | C |
| ATOM | 6191 | O | ILE H | 395 | −9.242 | −4.353 | −23.312 | 1.00 | 13.45 | B | O |
| ATOM | 6192 | N | MET H | 396 | −9.462 | −6.054 | −21.877 | 1.00 | 13.06 | B | N |
| ATOM | 6193 | CA | MET H | 396 | −8.356 | −6.773 | −22.482 | 1.00 | 13.00 | B | C |
| ATOM | 6194 | CB | MET H | 396 | −7.104 | −6.717 | −21.605 | 1.00 | 13.34 | B | C |
| ATOM | 6195 | CG | MET H | 396 | −7.052 | −7.752 | −20.500 | 1.00 | 14.30 | B | C |
| ATOM | 6196 | SD | MET H | 396 | −5.571 | −7.581 | −19.484 | 1.00 | 18.50 | B | S |
| ATOM | 6197 | CE | MET H | 396 | −5.752 | −5.895 | −18.904 | 1.00 | 17.80 | B | C |
| ATOM | 6198 | C | MET H | 396 | −8.702 | −8.200 | −22.867 | 1.00 | 12.96 | B | C |
| ATOM | 6199 | O | MET H | 396 | −9.615 | −8.808 | −22.309 | 1.00 | 13.04 | B | O |
| ATOM | 6200 | N | THR H | 397 | −7.935 | −8.734 | −23.808 | 1.00 | 12.78 | B | N |
| ATOM | 6201 | CA | THR H | 397 | −8.350 | −9.888 | −24.585 | 1.00 | 12.51 | B | C |
| ATOM | 6202 | CB | THR H | 397 | −8.389 | −9.563 | −26.087 | 1.00 | 12.39 | B | C |
| ATOM | 6203 | OG1 | THR H | 397 | −9.593 | −8.851 | −26.394 | 1.00 | 12.50 | B | O |
| ATOM | 6204 | CG2 | THR H | 397 | −8.335 | −10.839 | −26.912 | 1.00 | 12.55 | B | C |
| ATOM | 6205 | C | THR H | 397 | −7.350 | −11.005 | −24.366 | 1.00 | 12.58 | B | C |
| ATOM | 6206 | O | THR H | 397 | −6.143 | −10.772 | −24.327 | 1.00 | 12.88 | B | O |
| ATOM | 6207 | N | SER H | 398 | −7.859 | −12.210 | −24.165 | 1.00 | 12.55 | B | N |
| ATOM | 6208 | CA | SER H | 398 | −7.082 | −13.401 | −24.410 | 1.00 | 12.76 | B | C |
| ATOM | 6209 | CB | SER H | 398 | −6.083 | −13.627 | −23.273 | 1.00 | 12.52 | B | C |
| ATOM | 6210 | OG | SER H | 398 | −5.764 | −15.001 | −23.133 | 1.00 | 12.72 | B | O |
| ATOM | 6211 | C | SER H | 398 | −8.070 | −14.539 | −24.456 | 1.00 | 12.99 | B | C |
| ATOM | 6212 | O | SER H | 398 | −8.838 | −14.672 | −25.410 | 1.00 | 13.13 | B | O |
| ATOM | 6213 | N | LYS H | 399 | −8.182 | −15.232 | −23.330 | 1.00 | 13.01 | B | N |
| ATOM | 6214 | CA | LYS H | 399 | −7.890 | −16.651 | −23.342 | 1.00 | 13.29 | B | C |
| ATOM | 6215 | CB | LYS H | 399 | −6.739 | −17.020 | −24.279 | 1.00 | 13.79 | B | C |
| ATOM | 6216 | CG | LYS H | 399 | −6.786 | −16.279 | −25.618 | 1.00 | 14.34 | B | C |
| ATOM | 6217 | CD | LYS H | 399 | −7.835 | −16.868 | −26.552 | 1.00 | 16.43 | B | C |
| ATOM | 6218 | CE | LYS H | 399 | −7.864 | −16.139 | −27.889 | 1.00 | 17.37 | B | C |
| ATOM | 6219 | NZ | LYS H | 399 | −8.770 | −16.802 | −28.867 | 1.00 | 16.62 | B | N |
| ATOM | 6220 | C | LYS H | 399 | −8.128 | −17.609 | −22.171 | 1.00 | 13.09 | B | C |
| ATOM | 6221 | O | LYS H | 399 | −9.087 | −18.376 | −22.216 | 1.00 | 12.87 | B | O |
| ATOM | 6222 | N | THR H | 400 | −7.140 | −17.783 | −21.300 | 1.00 | 13.04 | B | N |
| ATOM | 6223 | CA | THR H | 400 | −7.133 | −18.953 | −20.420 | 1.00 | 13.38 | B | C |
| ATOM | 6224 | CB | THR H | 400 | −5.703 | −19.453 | −20.133 | 1.00 | 13.74 | B | C |
| ATOM | 6225 | OG1 | THR H | 400 | −4.773 | −18.763 | −20.977 | 1.00 | 14.38 | B | O |
| ATOM | 6226 | CG2 | THR H | 400 | −5.601 | −20.950 | −20.394 | 1.00 | 14.32 | B | C |
| ATOM | 6227 | C | THR H | 400 | −7.866 | −18.687 | −19.104 | 1.00 | 13.20 | B | C |
| ATOM | 6228 | O | THR H | 400 | −7.913 | −17.551 | −18.630 | 1.00 | 13.12 | B | O |
| ATOM | 6229 | N | ASP H | 401 | −8.478 | −19.728 | −18.545 | 1.00 | 12.97 | B | N |
| ATOM | 6230 | CA | ASP H | 401 | −8.896 | −19.715 | −17.145 | 1.00 | 12.50 | B | C |
| ATOM | 6231 | CB | ASP H | 401 | −9.622 | −21.022 | −16.798 | 1.00 | 12.73 | B | C |
| ATOM | 6232 | CG | ASP H | 401 | −10.854 | −21.261 | −17.651 | 1.00 | 13.98 | B | C |
| ATOM | 6233 | OD1 | ASP H | 401 | −10.870 | −20.793 | −18.808 | 1.00 | 16.55 | B | O |
| ATOM | 6234 | OD2 | ASP H | 401 | −11.708 | −22.076 | −17.236 | 1.00 | 14.80 | B | O |
| ATOM | 6235 | C | ASP H | 401 | −7.683 | −19.567 | −16.222 | 1.00 | 12.13 | B | C |
| ATOM | 6236 | O | ASP H | 401 | −6.906 | −20.508 | −16.071 | 1.00 | 12.31 | B | O |
| ATOM | 6237 | N | VAL H | 402 | −7.609 | −18.459 | −15.489 | 1.00 | 11.49 | B | N |
| ATOM | 6238 | CA | VAL H | 402 | −7.400 | −18.523 | −14.044 | 1.00 | 10.74 | B | C |
| ATOM | 6239 | CB | VAL H | 402 | −7.004 | −17.151 | −13.456 | 1.00 | 10.60 | B | C |
| ATOM | 6240 | CG1 | VAL H | 402 | −7.433 | −17.045 | −11.998 | 1.00 | 11.26 | B | C |
| ATOM | 6241 | CG2 | VAL H | 402 | −5.504 | −16.918 | −13.602 | 1.00 | 10.99 | B | C |
| ATOM | 6242 | C | VAL H | 402 | −8.707 | −18.986 | −13.423 | 1.00 | 10.24 | B | C |
| ATOM | 6243 | O | VAL H | 402 | −9.515 | −19.616 | −14.104 | 1.00 | 10.30 | B | O |
| ATOM | 6244 | N | SER H | 403 | −9.015 | −18.516 | −12.218 | 1.00 | 9.76 | B | N |
| ATOM | 6245 | CA | SER H | 403 | −10.286 | −18.897 | −11.605 | 1.00 | 9.18 | B | C |
| ATOM | 6246 | CB | SER H | 403 | −10.485 | −20.406 | −11.726 | 1.00 | 9.41 | B | C |
| ATOM | 6247 | OG | SER H | 403 | −9.349 | −21.015 | −12.313 | 1.00 | 10.82 | B | O |
| ATOM | 6248 | C | SER H | 403 | −10.528 | −18.449 | −10.162 | 1.00 | 8.51 | B | C |
| ATOM | 6249 | O | SER H | 403 | −9.701 | −18.699 | −9.285 | 1.00 | 8.30 | B | O |
| ATOM | 6250 | N | SER H | 404 | −11.773 | −18.053 | −9.888 | 1.00 | 8.35 | B | N |
| ATOM | 6251 | CA | SER H | 404 | −12.352 | −18.148 | −8.541 | 1.00 | 8.18 | B | C |
| ATOM | 6252 | CB | SER H | 404 | −11.585 | −17.257 | −7.564 | 1.00 | 8.18 | B | C |
| ATOM | 6253 | OG | SER H | 404 | −12.250 | −16.019 | −7.382 | 1.00 | 8.83 | B | O |
| ATOM | 6254 | C | SER H | 404 | −13.841 | −17.789 | −8.505 | 1.00 | 7.62 | B | C |
| ATOM | 6255 | O | SER H | 404 | −14.505 | −17.759 | −9.541 | 1.00 | 7.57 | B | O |
| ATOM | 6256 | N | SER H | 405 | −14.357 | −17.506 | −7.310 | 1.00 | 7.08 | B | N |
| ATOM | 6257 | CA | SER H | 405 | −15.777 | −17.210 | −7.151 | 1.00 | 6.53 | B | C |
| ATOM | 6258 | CB | SER H | 405 | −16.568 | −18.475 | −6.802 | 1.00 | 6.42 | B | C |
| ATOM | 6259 | OG | SER H | 405 | −17.786 | −18.156 | −6.146 | 1.00 | 5.66 | B | O |
| ATOM | 6260 | C | SER H | 405 | −16.070 | −16.101 | −6.144 | 1.00 | 6.28 | B | C |
| ATOM | 6261 | O | SER H | 405 | −15.289 | −15.847 | −5.226 | 1.00 | 5.98 | B | O |
| ATOM | 6262 | N | VAL H | 406 | −17.254 | −15.514 | −6.281 | 1.00 | 6.22 | B | N |
| ATOM | 6263 | CA | VAL H | 406 | −17.629 | −14.309 | −5.557 | 1.00 | 6.50 | B | C |
| ATOM | 6264 | CB | VAL H | 406 | −17.390 | −13.053 | −6.414 | 1.00 | 6.45 | B | C |
| ATOM | 6265 | CG1 | VAL H | 406 | −17.384 | −11.811 | −5.546 | 1.00 | 7.66 | B | C |
| ATOM | 6266 | CG2 | VAL H | 406 | −16.090 | −13.177 | −7.191 | 1.00 | 7.38 | B | C |
| ATOM | 6267 | C | VAL H | 406 | −19.115 | −14.377 | −5.237 | 1.00 | 6.24 | B | C |
| ATOM | 6268 | O | VAL H | 406 | −19.946 | −14.488 | −6.138 | 1.00 | 6.14 | B | O |
| ATOM | 6269 | N | ILE H | 407 | −19.450 | −14.313 | −3.955 | 1.00 | 6.32 | B | N |
| ATOM | 6270 | CA | ILE H | 407 | −20.831 | −14.478 | −3.534 | 1.00 | 6.60 | B | C |
| ATOM | 6271 | CB | ILE H | 407 | −20.925 | −15.154 | −2.163 | 1.00 | 6.27 | B | C |
| ATOM | 6272 | CG1 | ILE H | 407 | −20.643 | −16.651 | −2.296 | 1.00 | 6.19 | B | C |
| ATOM | 6273 | CD1 | ILE H | 407 | −21.477 | −17.514 | −1.376 | 1.00 | 8.34 | B | C |
| ATOM | 6274 | CG2 | ILE H | 407 | −22.294 | −14.923 | −1.550 | 1.00 | 6.42 | B | C |
| ATOM | 6275 | C | ILE H | 407 | −21.570 | −13.149 | −3.497 | 1.00 | 7.20 | B | C |
| ATOM | 6276 | O | ILE H | 407 | −21.094 | −12.176 | −2.913 | 1.00 | 7.42 | B | O |
| ATOM | 6277 | N | THR H | 408 | −22.792 | −13.155 | −4.016 | 1.00 | 7.57 | B | N |
| ATOM | 6278 | CA | THR H | 408 | −23.563 | −11.933 | −4.148 | 1.00 | 8.19 | B | C |
| ATOM | 6279 | CB | THR H | 408 | −24.066 | −11.739 | −5.587 | 1.00 | 8.15 | B | C |
| ATOM | 6280 | OG1 | THR H | 408 | −24.863 | −12.866 | −5.971 | 1.00 | 8.88 | B | O |
| ATOM | 6281 | CG2 | THR H | 408 | −22.893 | −11.606 | −6.542 | 1.00 | 7.93 | B | C |
| ATOM | 6282 | C | THR H | 408 | −24.753 | −11.933 | −3.203 | 1.00 | 8.55 | B | C |
| ATOM | 6283 | O | THR H | 408 | −24.998 | −12.905 | −2.488 | 1.00 | 9.07 | B | O |
| ATOM | 6284 | N | SER H | 409 | −25.513 | −10.844 | −3.238 | 1.00 | 8.70 | B | N |
| ATOM | 6285 | CA | SER H | 409 | −26.644 | −10.667 | −2.344 | 1.00 | 9.20 | B | C |
| ATOM | 6286 | CB | SER H | 409 | −27.266 | −9.284 | −2.543 | 1.00 | 9.72 | B | C |
| ATOM | 6287 | OG | SER H | 409 | −26.279 | −8.269 | −2.491 | 1.00 | 10.75 | B | O |
| ATOM | 6288 | C | SER H | 409 | −27.686 | −11.747 | −2.596 | 1.00 | 9.01 | B | C |
| ATOM | 6289 | O | SER H | 409 | −28.360 | −12.203 | −1.671 | 1.00 | 9.02 | B | O |
| ATOM | 6290 | N | LEU H | 410 | −27.804 | −12.160 | −3.853 | 1.00 | 8.91 | B | N |
| ATOM | 6291 | CA | LEU H | 410 | −28.962 | −12.921 | −4.302 | 1.00 | 9.29 | B | C |
| ATOM | 6292 | CB | LEU H | 410 | −29.901 | −12.033 | −5.118 | 1.00 | 9.61 | B | C |
| ATOM | 6293 | CG | LEU H | 410 | −31.317 | −11.873 | −4.558 | 1.00 | 9.70 | B | C |
| ATOM | 6294 | CD1 | LEU H | 410 | −31.289 | −11.183 | −3.203 | 1.00 | 11.11 | B | C |
| ATOM | 6295 | CD2 | LEU H | 410 | −32.204 | −11.113 | −5.532 | 1.00 | 9.16 | B | C |
| ATOM | 6296 | C | LEU H | 410 | −28.533 | −14.134 | −5.119 | 1.00 | 9.12 | B | C |
| ATOM | 6297 | O | LEU H | 410 | −29.364 | −14.840 | −5.690 | 1.00 | 9.08 | B | O |
| ATOM | 6298 | N | GLY H | 411 | −27.228 | −14.365 | −5.173 | 1.00 | 9.03 | B | N |
| ATOM | 6299 | CA | GLY H | 411 | −26.691 | −15.606 | −5.706 | 1.00 | 8.44 | B | C |
| ATOM | 6300 | C | GLY H | 411 | −25.180 | −15.615 | −5.632 | 1.00 | 7.91 | B | C |
| ATOM | 6301 | O | GLY H | 411 | −24.594 | −15.123 | −4.668 | 1.00 | 7.85 | B | O |
| ATOM | 6302 | N | ALA H | 412 | −24.546 | −16.095 | −6.694 | 1.00 | 7.30 | B | N |
| ATOM | 6303 | CA | ALA H | 412 | −23.095 | −16.167 | −6.738 | 1.00 | 6.92 | B | C |
| ATOM | 6304 | CB | ALA H | 412 | −22.607 | −17.439 | −6.062 | 1.00 | 6.90 | B | C |
| ATOM | 6305 | C | ALA H | 412 | −22.593 | −16.098 | −8.173 | 1.00 | 6.99 | B | C |
| ATOM | 6306 | O | ALA H | 412 | −23.277 | −16.525 | −9.102 | 1.00 | 6.94 | B | O |
| ATOM | 6307 | N | ILE H | 413 | −21.415 | −15.510 | −8.349 | 1.00 | 7.29 | B | N |
| ATOM | 6308 | CA | ILE H | 413 | −20.705 | −15.567 | −9.622 | 1.00 | 7.49 | B | C |
| ATOM | 6309 | CB | ILE H | 413 | −20.073 | −14.207 | −9.961 | 1.00 | 7.24 | B | C |
| ATOM | 6310 | CG1 | ILE H | 413 | −21.142 | −13.236 | −10.466 | 1.00 | 7.18 | B | C |
| ATOM | 6311 | CD1 | ILE H | 413 | −20.589 | −12.045 | −11.233 | 1.00 | 7.70 | B | C |
| ATOM | 6312 | CG2 | ILE H | 413 | −18.958 | −14.377 | −10.983 | 1.00 | 7.70 | B | C |
| ATOM | 6313 | C | ILE H | 413 | −19.596 | −16.608 | −9.554 | 1.00 | 7.94 | B | C |
| ATOM | 6314 | O | ILE H | 413 | −18.965 | −16.775 | −8.512 | 1.00 | 8.39 | B | O |
| ATOM | 6315 | N | VAL H | 414 | −19.276 | −17.221 | −10.687 | 1.00 | 8.22 | B | N |
| ATOM | 6316 | CA | VAL H | 414 | −18.141 | −18.134 | −10.734 | 1.00 | 8.77 | B | C |
| ATOM | 6317 | CB | VAL H | 414 | −18.558 | −19.593 | −10.482 | 1.00 | 8.57 | B | C |
| ATOM | 6318 | CG1 | VAL H | 414 | −19.806 | −19.936 | −11.279 | 1.00 | 9.12 | B | C |
| ATOM | 6319 | CG2 | VAL H | 414 | −17.416 | −20.538 | −10.825 | 1.00 | 8.90 | B | C |
| ATOM | 6320 | C | VAL H | 414 | −17.323 | −18.031 | −12.015 | 1.00 | 8.92 | B | C |
| ATOM | 6321 | O | VAL H | 414 | −17.803 | −18.347 | −13.105 | 1.00 | 9.00 | B | O |
| ATOM | 6322 | N | SER H | 415 | −16.058 | −17.659 | −11.853 | 1.00 | 8.97 | B | N |
| ATOM | 6323 | CA | SER H | 415 | −15.120 | −17.594 | −12.962 | 1.00 | 9.02 | B | C |
| ATOM | 6324 | CB | SER H | 415 | −14.260 | −16.335 | −12.855 | 1.00 | 8.98 | B | C |
| ATOM | 6325 | OG | SER H | 415 | −15.043 | −15.218 | −12.473 | 1.00 | 9.54 | B | O |
| ATOM | 6326 | C | SER H | 415 | −14.235 | −18.832 | −12.987 | 1.00 | 9.36 | B | C |
| ATOM | 6327 | O | SER H | 415 | −13.326 | −18.975 | −12.170 | 1.00 | 9.61 | B | O |
| ATOM | 6328 | N | CYS H | 416 | −14.602 | −19.786 | −13.834 | 1.00 | 9.57 | B | N |
| ATOM | 6329 | CA | CYS H | 416 | −13.851 | −21.023 | −13.963 | 1.00 | 9.61 | B | C |
| ATOM | 6330 | CB | CYS H | 416 | −14.801 | −22.216 | −14.038 | 1.00 | 9.62 | B | C |
| ATOM | 6331 | SG | CYS H | 416 | −14.118 | −23.727 | −13.341 | 1.00 | 11.33 | B | S |
| ATOM | 6332 | C | CYS H | 416 | −12.998 | −20.973 | −15.215 | 1.00 | 9.61 | B | C |
| ATOM | 6333 | O | CYS H | 416 | −13.519 | −20.829 | −16.320 | 1.00 | 9.80 | B | O |
| ATOM | 6334 | N | TYR H | 417 | −11.684 | −20.941 | −15.027 | 1.00 | 9.48 | B | N |
| ATOM | 6335 | CA | TYR H | 417 | −10.772 | −20.739 | −16.142 | 1.00 | 9.28 | B | C |
| ATOM | 6336 | CB | TYR H | 417 | −10.307 | −19.281 | −16.205 | 1.00 | 9.17 | B | C |
| ATOM | 6337 | CG | TYR H | 417 | −11.358 | −18.318 | −16.714 | 1.00 | 8.71 | B | C |
| ATOM | 6338 | CD1 | TYR H | 417 | −11.483 | −17.048 | −16.167 | 1.00 | 8.54 | B | C |
| ATOM | 6339 | CE1 | TYR H | 417 | −12.425 | −16.159 | −16.635 | 1.00 | 9.56 | B | C |
| ATOM | 6340 | CZ | TYR H | 417 | −13.230 | −16.516 | −17.693 | 1.00 | 9.83 | B | C |
| ATOM | 6341 | OH | TYR H | 417 | −14.184 | −15.638 | −18.143 | 1.00 | 10.51 | B | O |
| ATOM | 6342 | CE2 | TYR H | 417 | −13.122 | −17.765 | −18.263 | 1.00 | 9.36 | B | C |
| ATOM | 6343 | CD2 | TYR H | 417 | −12.177 | −18.651 | −17.786 | 1.00 | 8.95 | B | C |
| ATOM | 6344 | C | TYR H | 417 | −9.572 | −21.677 | −16.068 | 1.00 | 9.35 | B | C |
| ATOM | 6345 | O | TYR H | 417 | −9.336 | −22.318 | −15.044 | 1.00 | 9.11 | B | O |
| ATOM | 6346 | N | GLY H | 418 | −8.830 | −21.762 | −17.169 | 1.00 | 9.71 | B | N |
| ATOM | 6347 | CA | GLY H | 418 | −7.678 | −22.656 | −17.263 | 1.00 | 9.95 | B | C |
| ATOM | 6348 | C | GLY H | 418 | −8.045 | −24.101 | −16.993 | 1.00 | 10.09 | B | C |
| ATOM | 6349 | O | GLY H | 418 | −9.082 | −24.584 | −17.450 | 1.00 | 10.17 | B | O |
| ATOM | 6350 | N | LYS H | 419 | −7.251 | −24.756 | −16.154 | 1.00 | 10.17 | B | N |
| ATOM | 6351 | CA | LYS H | 419 | −7.426 | −26.177 | −15.896 | 1.00 | 10.50 | B | C |
| ATOM | 6352 | CB | LYS H | 419 | −6.086 | −26.827 | −15.545 | 1.00 | 11.09 | B | C |
| ATOM | 6353 | CG | LYS H | 419 | −5.332 | −27.389 | −16.738 | 1.00 | 12.94 | B | C |
| ATOM | 6354 | CD | LYS H | 419 | −4.265 | −28.377 | −16.297 | 1.00 | 15.52 | B | C |
| ATOM | 6355 | CE | LYS H | 419 | −3.705 | −29.152 | −17.479 | 1.00 | 17.07 | B | C |
| ATOM | 6356 | NZ | LYS H | 419 | −3.179 | −28.247 | −18.538 | 1.00 | 19.30 | B | N |
| ATOM | 6357 | C | LYS H | 419 | −8.447 | −26.465 | −14.798 | 1.00 | 10.23 | B | C |
| ATOM | 6358 | O | LYS H | 419 | −8.613 | −27.616 | −14.398 | 1.00 | 10.58 | B | O |
| ATOM | 6359 | N | THR H | 420 | −9.070 | −25.426 | −14.250 | 1.00 | 10.10 | B | N |
| ATOM | 6360 | CA | THR H | 420 | −9.805 | −25.587 | −12.997 | 1.00 | 9.74 | B | C |
| ATOM | 6361 | CB | THR H | 420 | −9.938 | −24.270 | −12.223 | 1.00 | 9.28 | B | C |
| ATOM | 6362 | OG1 | THR H | 420 | −9.022 | −23.306 | −12.754 | 1.00 | 9.21 | B | O |
| ATOM | 6363 | CG2 | THR H | 420 | −9.633 | −24.499 | −10.752 | 1.00 | 9.03 | B | C |
| ATOM | 6364 | C | THR H | 420 | −11.181 | −26.223 | −13.168 | 1.00 | 9.95 | B | C |
| ATOM | 6365 | O | THR H | 420 | −11.779 | −26.160 | −14.242 | 1.00 | 10.35 | B | O |
| ATOM | 6366 | N | LYS H | 421 | −11.652 | −26.879 | −12.111 | 1.00 | 10.07 | B | N |
| ATOM | 6367 | CA | LYS H | 421 | −12.961 | −27.527 | −12.115 | 1.00 | 10.21 | B | C |
| ATOM | 6368 | CB | LYS H | 421 | −12.835 | −28.979 | −11.649 | 1.00 | 10.30 | B | C |
| ATOM | 6369 | CG | LYS H | 421 | −12.705 | −29.989 | −12.776 | 1.00 | 12.43 | B | C |
| ATOM | 6370 | CD | LYS H | 421 | −12.482 | −31.391 | −12.234 | 1.00 | 15.12 | B | C |
| ATOM | 6371 | CE | LYS H | 421 | −11.769 | −32.267 | −13.251 | 1.00 | 15.21 | B | C |
| ATOM | 6372 | NZ | LYS H | 421 | −11.047 | −33.396 | −12.601 | 1.00 | 15.98 | B | N |
| ATOM | 6373 | C | LYS H | 421 | −13.933 | −26.782 | −11.206 | 1.00 | 10.09 | B | C |
| ATOM | 6374 | O | LYS H | 421 | −13.600 | −26.456 | −10.067 | 1.00 | 10.36 | B | O |
| ATOM | 6375 | N | CYS H | 422 | −15.155 | −26.574 | −11.685 | 1.00 | 9.81 | B | N |
| ATOM | 6376 | CA | CYS H | 422 | −16.153 | −25.863 | −10.898 | 1.00 | 9.56 | B | C |
| ATOM | 6377 | CB | CYS H | 422 | −16.222 | −24.392 | −11.302 | 1.00 | 9.72 | B | C |
| ATOM | 6378 | SG | CYS H | 422 | −14.611 | −23.591 | −11.379 | 1.00 | 11.42 | B | S |
| ATOM | 6379 | C | CYS H | 422 | −17.535 | −26.499 | −10.939 | 1.00 | 9.18 | B | C |
| ATOM | 6380 | O | CYS H | 422 | −17.952 | −27.056 | −11.954 | 1.00 | 9.27 | B | O |
| ATOM | 6381 | N | THR H | 423 | −18.273 | −26.311 | −9.851 | 1.00 | 8.93 | B | N |
| ATOM | 6382 | CA | THR H | 423 | −19.417 | −27.147 | −9.527 | 1.00 | 8.64 | B | C |
| ATOM | 6383 | CB | THR H | 423 | −19.014 | −28.315 | −8.606 | 1.00 | 8.32 | B | C |
| ATOM | 6384 | OG1 | THR H | 423 | −17.878 | −28.993 | −9.154 | 1.00 | 7.97 | B | O |
| ATOM | 6385 | CG2 | THR H | 423 | −20.165 | −29.297 | −8.456 | 1.00 | 8.36 | B | C |
| ATOM | 6386 | C | THR H | 423 | −20.441 | −26.297 | −8.791 | 1.00 | 8.86 | B | C |
| ATOM | 6387 | O | THR H | 423 | −20.133 | −25.694 | −7.763 | 1.00 | 9.26 | B | O |
| ATOM | 6388 | N | ALA H | 424 | −21.676 | −26.306 | −9.279 | 1.00 | 8.74 | B | N |
| ATOM | 6389 | CA | ALA H | 424 | −22.803 | −25.836 | −8.487 | 1.00 | 8.79 | B | C |
| ATOM | 6390 | CB | ALA H | 424 | −23.693 | −24.926 | −9.317 | 1.00 | 8.94 | B | C |
| ATOM | 6391 | C | ALA H | 424 | −23.605 | −27.008 | −7.932 | 1.00 | 8.84 | B | C |
| ATOM | 6392 | O | ALA H | 424 | −24.158 | −27.807 | −8.687 | 1.00 | 9.24 | B | O |
| ATOM | 6393 | N | SER H | 425 | −23.649 | −27.113 | −6.608 | 1.00 | 8.81 | B | N |
| ATOM | 6394 | CA | SER H | 425 | −24.412 | −28.166 | −5.950 | 1.00 | 8.85 | B | C |
| ATOM | 6395 | CB | SER H | 425 | −23.530 | −28.925 | −4.957 | 1.00 | 8.94 | B | C |
| ATOM | 6396 | OG | SER H | 425 | −22.166 | −28.871 | −5.341 | 1.00 | 7.69 | B | O |
| ATOM | 6397 | C | SER H | 425 | −25.626 | −27.584 | −5.235 | 1.00 | 9.01 | B | C |
| ATOM | 6398 | O | SER H | 425 | −25.593 | −26.447 | −4.763 | 1.00 | 8.63 | B | O |
| ATOM | 6399 | N | ASN H | 426 | −26.716 | −28.345 | −5.208 | 1.00 | 9.54 | B | N |
| ATOM | 6400 | CA | ASN H | 426 | −27.971 | −27.851 | −4.658 | 1.00 | 10.19 | B | C |
| ATOM | 6401 | CB | ASN H | 426 | −29.166 | −28.508 | −5.356 | 1.00 | 9.77 | B | C |
| ATOM | 6402 | CG | ASN H | 426 | −29.499 | −29.874 | −4.787 | 1.00 | 9.43 | B | C |
| ATOM | 6403 | OD1 | ASN H | 426 | −28.998 | −30.259 | −3.730 | 1.00 | 9.01 | B | O |
| ATOM | 6404 | ND2 | ASN H | 426 | −30.349 | −30.615 | −5.488 | 1.00 | 9.01 | B | N |
| ATOM | 6405 | C | ASN H | 426 | −28.074 | −28.031 | −3.146 | 1.00 | 11.16 | B | C |
| ATOM | 6406 | O | ASN H | 426 | −27.072 | −28.247 | −2.463 | 1.00 | 11.06 | B | O |
| ATOM | 6407 | N | LYS H | 427 | −29.285 | −27.847 | −2.628 | 1.00 | 12.38 | B | N |
| ATOM | 6408 | CA | LYS H | 427 | −29.569 | −27.993 | −1.203 | 1.00 | 13.52 | B | C |
| ATOM | 6409 | CB | LYS H | 427 | −31.082 | −28.017 | −0.968 | 1.00 | 14.00 | B | C |
| ATOM | 6410 | CG | LYS H | 427 | −31.681 | −26.675 | −0.585 | 1.00 | 16.18 | B | C |
| ATOM | 6411 | CD | LYS H | 427 | −33.105 | −26.838 | −0.078 | 1.00 | 18.24 | B | C |
| ATOM | 6412 | CE | LYS H | 427 | −33.487 | −25.714 | 0.870 | 1.00 | 19.72 | B | C |
| ATOM | 6413 | NZ | LYS H | 427 | −32.873 | −25.888 | 2.216 | 1.00 | 21.43 | B | N |
| ATOM | 6414 | C | LYS H | 427 | −28.943 | −29.256 | −0.621 | 1.00 | 13.71 | B | C |
| ATOM | 6415 | O | LYS H | 427 | −28.329 | −29.220 | 0.446 | 1.00 | 13.63 | B | O |
| ATOM | 6416 | N | ASN H | 428 | −29.233 | −30.390 | −1.252 | 1.00 | 14.28 | B | N |
| ATOM | 6417 | CA | ASN H | 428 | −28.838 | −31.694 | −0.729 | 1.00 | 14.60 | B | C |
| ATOM | 6418 | CB | ASN H | 428 | −29.715 | −32.800 | −1.324 | 1.00 | 14.57 | B | C |
| ATOM | 6419 | CG | ASN H | 428 | −31.103 | −32.312 | −1.690 | 1.00 | 13.24 | B | C |
| ATOM | 6420 | OD1 | ASN H | 428 | −31.832 | −31.790 | −0.846 | 1.00 | 9.74 | B | O |
| ATOM | 6421 | ND2 | ASN H | 428 | −31.503 | −32.546 | −2.935 | 1.00 | 12.83 | B | N |
| ATOM | 6422 | C | ASN H | 428 | −27.367 | −32.009 | −0.984 | 1.00 | 14.83 | B | C |
| ATOM | 6423 | O | ASN H | 428 | −26.834 | −32.982 | −0.451 | 1.00 | 15.25 | B | O |
| ATOM | 6424 | N | ARG H | 429 | −26.755 | −31.260 | −1.895 | 1.00 | 14.78 | B | N |
| ATOM | 6425 | CA | ARG H | 429 | −25.364 | −31.488 | −2.275 | 1.00 | 14.74 | B | C |
| ATOM | 6426 | CB | ARG H | 429 | −24.626 | −32.268 | −1.184 | 1.00 | 14.93 | B | C |
| ATOM | 6427 | CG | ARG H | 429 | −24.252 | −31.435 | 0.031 | 1.00 | 16.26 | B | C |
| ATOM | 6428 | CD | ARG H | 429 | −22.869 | −30.821 | −0.119 | 1.00 | 19.79 | B | C |
| ATOM | 6429 | NE | ARG H | 429 | −22.634 | −29.760 | 0.857 | 1.00 | 22.68 | B | N |
| ATOM | 6430 | CZ | ARG H | 429 | −22.532 | −29.958 | 2.167 | 1.00 | 23.54 | B | C |
| ATOM | 6431 | NH1 | ARG H | 429 | −22.640 | −31.182 | 2.667 | 1.00 | 22.87 | B | N |
| ATOM | 6432 | NH2 | ARG H | 429 | −22.321 | −28.932 | 2.980 | 1.00 | 23.53 | B | N |
| ATOM | 6433 | C | ARG H | 429 | −25.252 | −32.217 | −3.611 | 1.00 | 14.35 | B | C |
| ATOM | 6434 | O | ARG H | 429 | −24.175 | −32.285 | −4.203 | 1.00 | 14.62 | B | O |
| ATOM | 6435 | N | GLY H | 430 | −26.373 | −32.752 | −4.085 | 1.00 | 13.67 | B | N |
| ATOM | 6436 | CA | GLY H | 430 | −26.519 | −33.087 | −5.496 | 1.00 | 12.80 | B | C |
| ATOM | 6437 | C | GLY H | 430 | −26.014 | −31.977 | −6.397 | 1.00 | 12.11 | B | C |
| ATOM | 6438 | O | GLY H | 430 | −26.326 | −30.805 | −6.188 | 1.00 | 12.18 | B | O |
| ATOM | 6439 | N | ILE H | 431 | −25.216 | −32.346 | −7.393 | 1.00 | 11.58 | B | N |
| ATOM | 6440 | CA | ILE H | 431 | −24.551 | −31.372 | −8.251 | 1.00 | 10.76 | B | C |
| ATOM | 6441 | CB | ILE H | 431 | −23.175 | −31.896 | −8.715 | 1.00 | 10.43 | B | C |
| ATOM | 6442 | CG1 | ILE H | 431 | −22.220 | −32.024 | −7.527 | 1.00 | 10.95 | B | C |
| ATOM | 6443 | CD1 | ILE H | 431 | −21.220 | −33.153 | −7.665 | 1.00 | 12.08 | B | C |
| ATOM | 6444 | CG2 | ILE H | 431 | −22.592 | −30.993 | −9.791 | 1.00 | 10.07 | B | C |
| ATOM | 6445 | C | ILE H | 431 | −25.406 | −31.085 | −9.484 | 1.00 | 10.68 | B | C |
| ATOM | 6446 | O | ILE H | 431 | −25.703 | −31.994 | −10.258 | 1.00 | 10.40 | B | O |
| ATOM | 6447 | N | ILE H | 432 | −25.852 | −29.840 | −9.636 | 1.00 | 10.90 | B | N |
| ATOM | 6448 | CA | ILE H | 432 | −26.692 | −29.481 | −10.779 | 1.00 | 11.42 | B | C |
| ATOM | 6449 | CB | ILE H | 432 | −27.709 | −28.375 | −10.448 | 1.00 | 11.63 | B | C |
| ATOM | 6450 | CG1 | ILE H | 432 | −27.875 | −28.224 | −8.936 | 1.00 | 12.68 | B | C |
| ATOM | 6451 | CD1 | ILE H | 432 | −28.514 | −26.915 | −8.524 | 1.00 | 13.89 | B | C |
| ATOM | 6452 | CG2 | ILE H | 432 | −29.046 | −28.664 | −11.116 | 1.00 | 12.24 | B | C |
| ATOM | 6453 | C | ILE H | 432 | −25.884 | −29.045 | −11.993 | 1.00 | 11.41 | B | C |
| ATOM | 6454 | O | ILE H | 432 | −26.225 | −29.395 | −13.123 | 1.00 | 11.63 | B | O |
| ATOM | 6455 | N | LYS H | 433 | −24.982 | −28.093 | −11.782 | 1.00 | 11.52 | B | N |
| ATOM | 6456 | CA | LYS H | 433 | −24.226 | −27.523 | −12.888 | 1.00 | 11.96 | B | C |
| ATOM | 6457 | CB | LYS H | 433 | −24.433 | −26.010 | −12.976 | 1.00 | 12.31 | B | C |
| ATOM | 6458 | CG | LYS H | 433 | −23.850 | −25.387 | −14.237 | 1.00 | 14.42 | B | C |
| ATOM | 6459 | CD | LYS H | 433 | −24.649 | −24.175 | −14.685 | 1.00 | 16.74 | B | C |
| ATOM | 6460 | CE | LYS H | 433 | −24.112 | −23.613 | −15.993 | 1.00 | 17.08 | B | C |
| ATOM | 6461 | NZ | LYS H | 433 | −24.993 | −23.949 | −17.146 | 1.00 | 17.30 | B | N |
| ATOM | 6462 | C | LYS H | 433 | −22.743 | −27.850 | −12.793 | 1.00 | 11.65 | B | C |
| ATOM | 6463 | O | LYS H | 433 | −22.248 | −28.250 | −11.740 | 1.00 | 11.48 | B | O |
| ATOM | 6464 | N | THR H | 434 | −22.052 | −27.731 | −13.921 | 1.00 | 11.28 | B | N |
| ATOM | 6465 | CA | THR H | 434 | −20.617 | −27.970 | −13.975 | 1.00 | 11.21 | B | C |
| ATOM | 6466 | CB | THR H | 434 | −20.308 | −29.467 | −14.165 | 1.00 | 11.30 | B | C |
| ATOM | 6467 | OG1 | THR H | 434 | −18.945 | −29.629 | −14.574 | 1.00 | 11.83 | B | O |
| ATOM | 6468 | CG2 | THR H | 434 | −21.224 | −30.071 | −15.220 | 1.00 | 11.84 | B | C |
| ATOM | 6469 | C | THR H | 434 | −20.009 | −27.185 | −15.131 | 1.00 | 10.94 | B | C |
| ATOM | 6470 | O | THR H | 434 | −20.623 | −27.060 | −16.190 | 1.00 | 10.93 | B | O |
| ATOM | 6471 | N | PHE H | 435 | −18.867 | −26.549 | −14.886 | 1.00 | 11.10 | B | N |
| ATOM | 6472 | CA | PHE H | 435 | −18.610 | −25.235 | −15.464 | 1.00 | 11.96 | B | C |
| ATOM | 6473 | CB | PHE H | 435 | −18.186 | −24.223 | −14.401 | 1.00 | 11.51 | B | C |
| ATOM | 6474 | CG | PHE H | 435 | −19.344 | −23.561 | −13.710 | 1.00 | 11.19 | B | C |
| ATOM | 6475 | CD1 | PHE H | 435 | −19.580 | −23.788 | −12.365 | 1.00 | 10.86 | B | C |
| ATOM | 6476 | CE1 | PHE H | 435 | −20.692 | −23.253 | −11.741 | 1.00 | 10.24 | B | C |
| ATOM | 6477 | CZ | PHE H | 435 | −21.613 | −22.532 | −12.474 | 1.00 | 9.89 | B | C |
| ATOM | 6478 | CE2 | PHE H | 435 | −21.420 | −22.347 | −13.828 | 1.00 | 10.78 | B | C |
| ATOM | 6479 | CD2 | PHE H | 435 | −20.304 | −22.884 | −14.445 | 1.00 | 11.14 | B | C |
| ATOM | 6480 | C | PHE H | 435 | −17.694 | −25.202 | −16.684 | 1.00 | 13.00 | B | C |
| ATOM | 6481 | O | PHE H | 435 | −16.525 | −25.587 | −16.618 | 1.00 | 13.03 | B | O |
| ATOM | 6482 | N | SER H | 436 | −18.199 | −24.569 | −17.738 | 1.00 | 13.89 | B | N |
| ATOM | 6483 | CA | SER H | 436 | −17.549 | −24.549 | −19.039 | 1.00 | 14.67 | B | C |
| ATOM | 6484 | CB | SER H | 436 | −18.601 | −24.438 | −20.145 | 1.00 | 15.04 | B | C |
| ATOM | 6485 | OG | SER H | 436 | −18.532 | −25.539 | −21.033 | 1.00 | 16.58 | B | O |
| ATOM | 6486 | C | SER H | 436 | −16.586 | −23.371 | −19.134 | 1.00 | 14.75 | B | C |
| ATOM | 6487 | O | SER H | 436 | −16.849 | −22.398 | −19.841 | 1.00 | 14.75 | B | O |
| ATOM | 6488 | N | ASN H | 437 | −15.480 | −23.451 | −18.405 | 1.00 | 14.44 | B | N |
| ATOM | 6489 | CA | ASN H | 437 | −14.306 | −22.659 | −18.736 | 1.00 | 14.35 | B | C |
| ATOM | 6490 | CB | ASN H | 437 | −13.508 | −23.354 | −19.840 | 1.00 | 14.75 | B | C |
| ATOM | 6491 | CG | ASN H | 437 | −12.101 | −22.822 | −19.966 | 1.00 | 15.51 | B | C |
| ATOM | 6492 | OD1 | ASN H | 437 | −11.290 | −22.960 | −19.049 | 1.00 | 16.85 | B | O |
| ATOM | 6493 | ND2 | ASN H | 437 | −11.757 | −22.358 | −21.162 | 1.00 | 16.10 | B | N |
| ATOM | 6494 | C | ASN H | 437 | −14.692 | −21.239 | −19.161 | 1.00 | 13.69 | B | C |
| ATOM | 6495 | O | ASN H | 437 | −14.402 | −20.814 | −20.280 | 1.00 | 13.40 | B | O |
| ATOM | 6496 | N | GLY H | 438 | −15.374 | −20.527 | −18.267 | 1.00 | 13.39 | B | N |
| ATOM | 6497 | CA | GLY H | 438 | −15.942 | −19.216 | −18.573 | 1.00 | 12.92 | B | C |
| ATOM | 6498 | C | GLY H | 438 | −16.310 | −18.455 | −17.311 | 1.00 | 12.72 | B | C |
| ATOM | 6499 | O | GLY H | 438 | −15.574 | −18.495 | −16.324 | 1.00 | 12.83 | B | O |
| ATOM | 6500 | N | CYS H | 439 | −17.502 | −17.863 | −17.297 | 1.00 | 12.17 | B | N |
| ATOM | 6501 | CA | CYS H | 439 | −17.969 | −17.110 | −16.134 | 1.00 | 11.52 | B | C |
| ATOM | 6502 | CB | CYS H | 439 | −17.353 | −15.712 | −16.124 | 1.00 | 11.68 | B | C |
| ATOM | 6503 | SG | CYS H | 439 | −18.500 | −14.404 | −16.619 | 1.00 | 12.89 | B | S |
| ATOM | 6504 | C | CYS H | 439 | −19.492 | −17.006 | −16.079 | 1.00 | 10.95 | B | C |
| ATOM | 6505 | O | CYS H | 439 | −20.102 | −16.298 | −16.880 | 1.00 | 10.77 | B | O |
| ATOM | 6506 | N | ASP H | 440 | −20.089 | −17.630 | −15.066 | 1.00 | 10.41 | B | N |
| ATOM | 6507 | CA | ASP H | 440 | −21.541 | −17.762 | −14.987 | 1.00 | 10.06 | B | C |
| ATOM | 6508 | CB | ASP H | 440 | −21.953 | −19.232 | −15.091 | 1.00 | 10.85 | B | C |
| ATOM | 6509 | CG | ASP H | 440 | −22.561 | −19.576 | −16.438 | 1.00 | 13.11 | B | C |
| ATOM | 6510 | OD1 | ASP H | 440 | −23.750 | −19.260 | −16.654 | 1.00 | 14.97 | B | O |
| ATOM | 6511 | OD2 | ASP H | 440 | −21.872 | −20.227 | −17.251 | 1.00 | 15.27 | B | O |
| ATOM | 6512 | C | ASP H | 440 | −22.098 | −17.161 | −13.700 | 1.00 | 9.01 | B | C |
| ATOM | 6513 | O | ASP H | 440 | −21.345 | −16.790 | −12.798 | 1.00 | 9.09 | B | O |
| ATOM | 6514 | N | TYR H | 441 | −23.422 | −17.184 | −13.580 | 1.00 | 7.92 | B | N |
| ATOM | 6515 | CA | TYR H | 441 | −24.117 | −16.707 | −12.387 | 1.00 | 7.24 | B | C |
| ATOM | 6516 | CB | TYR H | 441 | −24.810 | −15.371 | −12.683 | 1.00 | 7.04 | B | C |
| ATOM | 6517 | CG | TYR H | 441 | −25.669 | −14.849 | −11.556 | 1.00 | 6.77 | B | C |
| ATOM | 6518 | CD1 | TYR H | 441 | −25.145 | −13.986 | −10.605 | 1.00 | 7.41 | B | C |
| ATOM | 6519 | CE1 | TYR H | 441 | −25.927 | −13.498 | −9.579 | 1.00 | 6.79 | B | C |
| ATOM | 6520 | CZ | TYR H | 441 | −27.270 | −13.809 | −9.539 | 1.00 | 6.49 | B | C |
| ATOM | 6521 | OH | TYR H | 441 | −28.049 | −13.340 | −8.507 | 1.00 | 5.09 | B | O |
| ATOM | 6522 | CE2 | TYR H | 441 | −27.823 | −14.634 | −10.494 | 1.00 | 7.52 | B | C |
| ATOM | 6523 | CD2 | TYR H | 441 | −27.029 | −15.122 | −11.512 | 1.00 | 7.12 | B | C |
| ATOM | 6524 | C | TYR H | 441 | −25.153 | −17.748 | −11.974 | 1.00 | 6.89 | B | C |
| ATOM | 6525 | O | TYR H | 441 | −25.720 | −18.436 | −12.823 | 1.00 | 7.12 | B | O |
| ATOM | 6526 | N | VAL H | 442 | −25.353 | −17.906 | −10.670 | 1.00 | 6.73 | B | N |
| ATOM | 6527 | CA | VAL H | 442 | −26.400 | −18.784 | −10.159 | 1.00 | 6.78 | B | C |
| ATOM | 6528 | CB | VAL H | 442 | −25.808 | −20.089 | −9.596 | 1.00 | 6.51 | B | C |
| ATOM | 6529 | CG1 | VAL H | 442 | −25.394 | −21.017 | −10.729 | 1.00 | 7.38 | B | C |
| ATOM | 6530 | CG2 | VAL H | 442 | −24.625 | −19.786 | −8.688 | 1.00 | 6.00 | B | C |
| ATOM | 6531 | C | VAL H | 442 | −27.167 | −18.062 | −9.059 | 1.00 | 7.13 | B | C |
| ATOM | 6532 | O | VAL H | 442 | −26.774 | −16.973 | −8.643 | 1.00 | 7.77 | B | O |
| ATOM | 6533 | N | SER H | 443 | −28.229 | −18.679 | −8.552 | 1.00 | 7.12 | B | N |
| ATOM | 6534 | CA | SER H | 443 | −29.017 | −18.039 | −7.504 | 1.00 | 7.64 | B | C |
| ATOM | 6535 | CB | SER H | 443 | −30.184 | −17.252 | −8.104 | 1.00 | 7.83 | B | C |
| ATOM | 6536 | OG | SER H | 443 | −31.411 | −17.936 | −7.922 | 1.00 | 7.99 | B | O |
| ATOM | 6537 | C | SER H | 443 | −29.508 | −18.989 | −6.415 | 1.00 | 8.16 | B | C |
| ATOM | 6538 | O | SER H | 443 | −29.421 | −20.210 | −6.552 | 1.00 | 8.69 | B | O |
| ATOM | 6539 | N | ASN H | 444 | −29.959 | −18.412 | −5.306 | 1.00 | 8.73 | B | N |
| ATOM | 6540 | CA | ASN H | 444 | −30.539 | −19.184 | −4.213 | 1.00 | 9.50 | B | C |
| ATOM | 6541 | CB | ASN H | 444 | −30.766 | −18.297 | −2.984 | 1.00 | 9.65 | B | C |
| ATOM | 6542 | CG | ASN H | 444 | −29.895 | −17.053 | −2.990 | 1.00 | 10.64 | B | C |
| ATOM | 6543 | OD1 | ASN H | 444 | −28.780 | −17.060 | −3.516 | 1.00 | 12.28 | B | O |
| ATOM | 6544 | ND2 | ASN H | 444 | −30.414 | −15.966 | −2.426 | 1.00 | 11.75 | B | N |
| ATOM | 6545 | C | ASN H | 444 | −31.843 | −19.863 | −4.618 | 1.00 | 10.24 | B | C |
| ATOM | 6546 | O | ASN H | 444 | −32.618 | −19.317 | −5.403 | 1.00 | 9.89 | B | O |
| ATOM | 6547 | N | LYS H | 445 | −32.146 | −20.975 | −3.955 | 1.00 | 11.63 | B | N |
| ATOM | 6548 | CA | LYS H | 445 | −32.826 | −22.105 | −4.588 | 1.00 | 13.07 | B | C |
| ATOM | 6549 | CB | LYS H | 445 | −34.344 | −21.990 | −4.421 | 1.00 | 13.70 | B | C |
| ATOM | 6550 | CG | LYS H | 445 | −35.131 | −23.200 | −4.915 | 1.00 | 15.80 | B | C |
| ATOM | 6551 | CD | LYS H | 445 | −34.354 | −24.501 | −4.753 | 1.00 | 18.31 | B | C |
| ATOM | 6552 | CE | LYS H | 445 | −33.508 | −24.498 | −3.490 | 1.00 | 19.03 | B | C |
| ATOM | 6553 | NZ | LYS H | 445 | −34.316 | −24.206 | −2.274 | 1.00 | 20.02 | B | N |
| ATOM | 6554 | C | LYS H | 445 | −32.459 | −22.265 | −6.060 | 1.00 | 12.96 | B | C |
| ATOM | 6555 | O | LYS H | 445 | −32.290 | −21.282 | −6.780 | 1.00 | 13.24 | B | O |
| ATOM | 6556 | N | GLY H | 446 | −32.350 | −23.513 | −6.504 | 1.00 | 13.08 | B | N |
| ATOM | 6557 | CA | GLY H | 446 | −31.215 | −23.936 | −7.312 | 1.00 | 12.42 | B | C |
| ATOM | 6558 | C | GLY H | 446 | −29.988 | −24.163 | −6.453 | 1.00 | 11.83 | B | C |
| ATOM | 6559 | O | GLY H | 446 | −29.855 | −25.209 | −5.819 | 1.00 | 12.44 | B | O |
| ATOM | 6560 | N | VAL H | 447 | −29.191 | −23.114 | −6.285 | 1.00 | 10.63 | B | N |
| ATOM | 6561 | CA | VAL H | 447 | −27.809 | −23.277 | −5.857 | 1.00 | 10.01 | B | C |
| ATOM | 6562 | CB | VAL H | 447 | −26.839 | −22.442 | −6.711 | 1.00 | 9.97 | B | C |
| ATOM | 6563 | CG1 | VAL H | 447 | −25.405 | −22.896 | −6.479 | 1.00 | 10.51 | B | C |
| ATOM | 6564 | CG2 | VAL H | 447 | −27.204 | −22.551 | −8.183 | 1.00 | 10.50 | B | C |
| ATOM | 6565 | C | VAL H | 447 | −27.603 | −22.962 | −4.380 | 1.00 | 9.53 | B | C |
| ATOM | 6566 | O | VAL H | 447 | −28.099 | −21.957 | −3.868 | 1.00 | 9.61 | B | O |
| ATOM | 6567 | N | ASP H | 448 | −26.719 | −23.735 | −3.760 | 1.00 | 9.16 | B | N |
| ATOM | 6568 | CA | ASP H | 448 | −26.522 | −23.714 | −2.318 | 1.00 | 8.75 | B | C |
| ATOM | 6569 | CB | ASP H | 448 | −27.139 | −24.971 | −1.698 | 1.00 | 9.22 | B | C |
| ATOM | 6570 | CG | ASP H | 448 | −27.482 | −24.795 | −0.234 | 1.00 | 11.44 | B | C |
| ATOM | 6571 | OD1 | ASP H | 448 | −26.877 | −23.921 | 0.421 | 1.00 | 13.83 | B | O |
| ATOM | 6572 | OD2 | ASP H | 448 | −28.319 | −25.568 | 0.276 | 1.00 | 14.13 | B | O |
| ATOM | 6573 | C | ASP H | 448 | −25.020 | −23.714 | −2.076 | 1.00 | 8.18 | B | C |
| ATOM | 6574 | O | ASP H | 448 | −24.528 | −23.151 | −1.098 | 1.00 | 8.06 | B | O |
| ATOM | 6575 | N | THR H | 449 | −24.298 | −24.280 | −3.036 | 1.00 | 7.63 | B | N |
| ATOM | 6576 | CA | THR H | 449 | −22.858 | −24.437 | −2.940 | 1.00 | 6.80 | B | C |
| ATOM | 6577 | CB | THR H | 449 | −22.485 | −25.824 | −2.386 | 1.00 | 6.89 | B | C |
| ATOM | 6578 | OG1 | THR H | 449 | −22.377 | −25.758 | −0.959 | 1.00 | 6.16 | B | O |
| ATOM | 6579 | CG2 | THR H | 449 | −21.163 | −26.296 | −2.970 | 1.00 | 7.55 | B | C |
| ATOM | 6580 | C | THR H | 449 | −22.265 | −24.297 | −4.333 | 1.00 | 6.53 | B | C |
| ATOM | 6581 | O | THR H | 449 | −22.828 | −24.792 | −5.309 | 1.00 | 6.59 | B | O |
| ATOM | 6582 | N | VAL H | 450 | −21.145 | −23.594 | −4.428 | 1.00 | 6.07 | B | N |
| ATOM | 6583 | CA | VAL H | 450 | −20.209 | −23.829 | −5.512 | 1.00 | 6.02 | B | C |
| ATOM | 6584 | CB | VAL H | 450 | −20.064 | −22.597 | −6.420 | 1.00 | 5.61 | B | C |
| ATOM | 6585 | CG1 | VAL H | 450 | −21.429 | −22.130 | −6.904 | 1.00 | 5.32 | B | C |
| ATOM | 6586 | CG2 | VAL H | 450 | −19.339 | −21.481 | −5.687 | 1.00 | 5.55 | B | C |
| ATOM | 6587 | C | VAL H | 450 | −18.853 | −24.210 | −4.950 | 1.00 | 6.37 | B | C |
| ATOM | 6588 | O | VAL H | 450 | −18.503 | −23.827 | −3.833 | 1.00 | 6.54 | B | O |
| ATOM | 6589 | N | SER H | 451 | −18.176 | −25.110 | −5.649 | 1.00 | 6.41 | B | N |
| ATOM | 6590 | CA | SER H | 451 | −16.786 | −25.397 | −5.362 | 1.00 | 6.70 | B | C |
| ATOM | 6591 | CB | SER H | 451 | −16.602 | −26.886 | −5.069 | 1.00 | 7.13 | B | C |
| ATOM | 6592 | OG | SER H | 451 | −17.729 | −27.629 | −5.498 | 1.00 | 9.26 | B | O |
| ATOM | 6593 | C | SER H | 451 | −15.944 | −24.990 | −6.555 | 1.00 | 6.47 | B | C |
| ATOM | 6594 | O | SER H | 451 | −16.363 | −25.147 | −7.701 | 1.00 | 6.54 | B | O |
| ATOM | 6595 | N | VAL H | 452 | −14.861 | −24.277 | −6.276 | 1.00 | 6.56 | B | N |
| ATOM | 6596 | CA | VAL H | 452 | −13.801 | −24.112 | −7.251 | 1.00 | 6.31 | B | C |
| ATOM | 6597 | CB | VAL H | 452 | −13.469 | −22.625 | −7.471 | 1.00 | 5.74 | B | C |
| ATOM | 6598 | CG1 | VAL H | 452 | −12.757 | −22.433 | −8.801 | 1.00 | 5.06 | B | C |
| ATOM | 6599 | CG2 | VAL H | 452 | −14.740 | −21.787 | −7.420 | 1.00 | 5.56 | B | C |
| ATOM | 6600 | C | VAL H | 452 | −12.558 | −24.861 | −6.791 | 1.00 | 6.60 | B | C |
| ATOM | 6601 | O | VAL H | 452 | −12.130 | −24.726 | −5.644 | 1.00 | 7.04 | B | O |
| ATOM | 6602 | N | GLY H | 453 | −12.047 | −25.732 | −7.654 | 1.00 | 6.54 | B | N |
| ATOM | 6603 | CA | GLY H | 453 | −11.013 | −26.672 | −7.250 | 1.00 | 6.43 | B | C |
| ATOM | 6604 | C | GLY H | 453 | −11.329 | −27.255 | −5.889 | 1.00 | 6.09 | B | C |
| ATOM | 6605 | O | GLY H | 453 | −12.396 | −27.836 | −5.690 | 1.00 | 5.90 | B | O |
| ATOM | 6606 | N | ASN H | 454 | −10.479 | −26.962 | −4.913 | 1.00 | 6.02 | B | N |
| ATOM | 6607 | CA | ASN H | 454 | −10.646 | −27.533 | −3.585 | 1.00 | 6.39 | B | C |
| ATOM | 6608 | CB | ASN H | 454 | −9.330 | −28.111 | −3.073 | 1.00 | 6.81 | B | C |
| ATOM | 6609 | CG | ASN H | 454 | −9.190 | −29.585 | −3.382 | 1.00 | 8.99 | B | C |
| ATOM | 6610 | OD1 | ASN H | 454 | −9.587 | −30.433 | −2.584 | 1.00 | 11.67 | B | O |
| ATOM | 6611 | ND2 | ASN H | 454 | −8.782 | −29.893 | −4.609 | 1.00 | 10.25 | B | N |
| ATOM | 6612 | C | ASN H | 454 | −11.235 | −26.563 | −2.573 | 1.00 | 6.15 | B | C |
| ATOM | 6613 | O | ASN H | 454 | −11.623 | −26.963 | −1.475 | 1.00 | 6.47 | B | O |
| ATOM | 6614 | N | THR H | 455 | −11.406 | −25.312 | −2.985 | 1.00 | 5.40 | B | N |
| ATOM | 6615 | CA | THR H | 455 | −12.129 | −24.347 | −2.172 | 1.00 | 4.53 | B | C |
| ATOM | 6616 | CB | THR H | 455 | −11.775 | −22.902 | −2.551 | 1.00 | 4.70 | B | C |
| ATOM | 6617 | OG1 | THR H | 455 | −10.351 | −22.746 | −2.567 | 1.00 | 5.28 | B | O |
| ATOM | 6618 | CG2 | THR H | 455 | −12.374 | −21.928 | −1.542 | 1.00 | 5.57 | B | C |
| ATOM | 6619 | C | THR H | 455 | −13.632 | −24.543 | −2.293 | 1.00 | 4.01 | B | C |
| ATOM | 6620 | O | THR H | 455 | −14.139 | −24.921 | −3.349 | 1.00 | 3.78 | B | O |
| ATOM | 6621 | N | LEU H | 456 | −14.338 | −24.306 | −1.194 | 1.00 | 3.87 | B | N |
| ATOM | 6622 | CA | LEU H | 456 | −15.787 | −24.419 | −1.181 | 1.00 | 4.05 | B | C |
| ATOM | 6623 | CB | LEU H | 456 | −16.232 | −25.520 | −0.219 | 1.00 | 3.95 | B | C |
| ATOM | 6624 | CG | LEU H | 456 | −17.708 | −25.915 | −0.306 | 1.00 | 3.76 | B | C |
| ATOM | 6625 | CD1 | LEU H | 456 | −18.042 | −26.440 | −1.694 | 1.00 | 5.35 | B | C |
| ATOM | 6626 | CD2 | LEU H | 456 | −18.053 | −26.944 | 0.758 | 1.00 | 4.10 | B | C |
| ATOM | 6627 | C | LEU H | 456 | −16.439 | −23.097 | −0.802 | 1.00 | 4.47 | B | C |
| ATOM | 6628 | O | LEU H | 456 | −16.186 | −22.551 | 0.272 | 1.00 | 4.94 | B | O |
| ATOM | 6629 | N | TYR H | 457 | −17.343 | −22.634 | −1.657 | 1.00 | 4.59 | B | N |
| ATOM | 6630 | CA | TYR H | 457 | −18.102 | −21.425 | −1.386 | 1.00 | 4.60 | B | C |
| ATOM | 6631 | CB | TYR H | 457 | −17.996 | −20.463 | −2.570 | 1.00 | 4.71 | B | C |
| ATOM | 6632 | CG | TYR H | 457 | −16.594 | −19.949 | −2.820 | 1.00 | 5.62 | B | C |
| ATOM | 6633 | CD1 | TYR H | 457 | −15.657 | −20.727 | −3.485 | 1.00 | 7.09 | B | C |
| ATOM | 6634 | CE1 | TYR H | 457 | −14.388 | −20.247 | −3.748 | 1.00 | 8.22 | B | C |
| ATOM | 6635 | CZ | TYR H | 457 | −14.029 | −18.994 | −3.305 | 1.00 | 8.06 | B | C |
| ATOM | 6636 | OH | TYR H | 457 | −12.763 | −18.520 | −3.550 | 1.00 | 8.80 | B | O |
| ATOM | 6637 | CE2 | TYR H | 457 | −14.933 | −18.212 | −2.622 | 1.00 | 7.18 | B | C |
| ATOM | 6638 | CD2 | TYR H | 457 | −16.203 | −18.694 | −2.375 | 1.00 | 6.66 | B | C |
| ATOM | 6639 | C | TYR H | 457 | −19.565 | −21.758 | −1.099 | 1.00 | 4.46 | B | C |
| ATOM | 6640 | O | TYR H | 457 | −20.180 | −22.551 | −1.811 | 1.00 | 4.92 | B | O |
| ATOM | 6641 | N | TYR H | 458 | −20.098 | −21.196 | −0.017 | 1.00 | 3.90 | B | N |
| ATOM | 6642 | CA | TYR H | 458 | −21.496 | −21.408 | 0.366 | 1.00 | 3.26 | B | C |
| ATOM | 6643 | CB | TYR H | 458 | −21.610 | −21.544 | 1.885 | 1.00 | 3.16 | B | C |
| ATOM | 6644 | CG | TYR H | 458 | −20.863 | −22.722 | 2.457 | 1.00 | 3.29 | B | C |
| ATOM | 6645 | CD1 | TYR H | 458 | −19.547 | −22.597 | 2.881 | 1.00 | 3.93 | B | C |
| ATOM | 6646 | CE1 | TYR H | 458 | −18.866 | −23.672 | 3.419 | 1.00 | 4.90 | B | C |
| ATOM | 6647 | CZ | TYR H | 458 | −19.501 | −24.890 | 3.535 | 1.00 | 5.12 | B | C |
| ATOM | 6648 | OH | TYR H | 458 | −18.826 | −25.967 | 4.062 | 1.00 | 5.42 | B | O |
| ATOM | 6649 | CE2 | TYR H | 458 | −20.810 | −25.036 | 3.128 | 1.00 | 4.72 | B | C |
| ATOM | 6650 | CD2 | TYR H | 458 | −21.483 | −23.955 | 2.598 | 1.00 | 3.89 | B | C |
| ATOM | 6651 | C | TYR H | 458 | −22.372 | −20.242 | −0.085 | 1.00 | 3.04 | B | C |
| ATOM | 6652 | O | TYR H | 458 | −22.185 | −19.119 | 0.378 | 1.00 | 3.37 | B | O |
| ATOM | 6653 | N | VAL H | 459 | −23.453 | −20.542 | −0.800 | 1.00 | 2.71 | B | N |
| ATOM | 6654 | CA | VAL H | 459 | −24.073 | −19.546 | −1.678 | 1.00 | 2.61 | B | C |
| ATOM | 6655 | CB | VAL H | 459 | −24.381 | −20.115 | −3.067 | 1.00 | 2.22 | B | C |
| ATOM | 6656 | CG1 | VAL H | 459 | −25.021 | −19.042 | −3.938 | 1.00 | 2.62 | B | C |
| ATOM | 6657 | CG2 | VAL H | 459 | −23.123 | −20.656 | −3.716 | 1.00 | 2.61 | B | C |
| ATOM | 6658 | C | VAL H | 459 | −25.372 | −18.924 | −1.190 | 1.00 | 3.03 | B | C |
| ATOM | 6659 | O | VAL H | 459 | −25.418 | −18.348 | −0.103 | 1.00 | 3.21 | B | O |
| ATOM | 6660 | N | ASN H | 460 | −26.215 | −18.648 | −2.185 | 1.00 | 106.93 | B | N |
| ATOM | 6661 | CA | ASN H | 460 | −27.625 | −18.431 | −1.941 | 1.00 | 95.54 | B | C |
| ATOM | 6662 | CB | ASN H | 460 | −27.810 | −17.878 | −0.542 | 1.00 | 20.00 | B | C |
| ATOM | 6663 | CG | ASN H | 460 | −28.967 | −18.507 | 0.162 | 1.00 | 20.00 | B | C |
| ATOM | 6664 | OD1 | ASN H | 460 | −29.655 | −19.349 | −0.416 | 1.00 | 20.00 | B | O |
| ATOM | 6665 | ND2 | ASN H | 460 | −29.359 | −17.912 | 1.280 | 1.00 | 20.00 | B | N |
| ATOM | 6666 | C | ASN H | 460 | −28.426 | −17.612 | −2.969 | 1.00 | 99.41 | B | C |
| ATOM | 6667 | O | ASN H | 460 | −29.222 | −18.178 | −3.718 | 1.00 | 108.91 | B | O |
| ATOM | 6668 | N | LYS H | 461 | −28.370 | −16.283 | −2.861 | 1.00 | 97.04 | B | N |
| ATOM | 6669 | CA | LYS H | 461 | −29.458 | −15.387 | −3.306 | 1.00 | 105.93 | B | C |
| ATOM | 6670 | CB | LYS H | 461 | −29.011 | −13.925 | −3.219 | 1.00 | 20.00 | B | C |
| ATOM | 6671 | CG | LYS H | 461 | −28.633 | −13.460 | −1.820 | 1.00 | 20.00 | B | C |
| ATOM | 6672 | CD | LYS H | 461 | −27.794 | −12.187 | −1.865 | 1.00 | 20.00 | B | C |
| ATOM | 6673 | CE | LYS H | 461 | −27.646 | −11.565 | −0.482 | 1.00 | 20.00 | B | C |
| ATOM | 6674 | NZ | LYS H | 461 | −26.746 | −10.375 | −0.482 | 1.00 | 20.00 | B | N |
| ATOM | 6675 | C | LYS H | 461 | −30.025 | −15.676 | −4.708 | 1.00 | 102.28 | B | C |
| ATOM | 6676 | O | LYS H | 461 | −29.779 | −16.739 | −5.276 | 1.00 | 105.02 | B | O |
| ATOM | 6677 | N | GLN H | 462 | −30.766 | −14.715 | −5.266 | 1.00 | 96.68 | B | N |
| ATOM | 6678 | CA | GLN H | 462 | −31.286 | −14.810 | −6.641 | 1.00 | 105.71 | B | C |
| ATOM | 6679 | CB | GLN H | 462 | −32.787 | −15.128 | −6.624 | 1.00 | 20.00 | B | C |
| ATOM | 6680 | CG | GLN H | 462 | −33.142 | −16.522 | −6.122 | 1.00 | 20.00 | B | C |
| ATOM | 6681 | CD | GLN H | 462 | −34.637 | −16.794 | −6.159 | 1.00 | 20.00 | B | C |
| ATOM | 6682 | OE1 | GLN H | 462 | −35.450 | −15.870 | −6.115 | 1.00 | 20.00 | B | O |
| ATOM | 6683 | NE2 | GLN H | 462 | −35.006 | −18.068 | −6.230 | 1.00 | 20.00 | B | N |
| ATOM | 6684 | C | GLN H | 462 | −31.052 | −13.514 | −7.429 | 1.00 | 101.50 | B | C |
| ATOM | 6685 | O | GLN H | 462 | −31.398 | −12.430 | −6.960 | 1.00 | 105.94 | B | O |
| ATOM | 6686 | N | GLU H | 463 | −30.565 | −13.634 | −8.663 | 1.00 | 98.92 | B | N |
| ATOM | 6687 | CA | GLU H | 463 | −30.241 | −12.453 | −9.465 | 1.00 | 108.59 | B | C |
| ATOM | 6688 | CB | GLU H | 463 | −28.792 | −12.505 | −9.966 | 1.00 | 20.00 | B | C |
| ATOM | 6689 | CG | GLU H | 463 | −27.767 | −11.973 | −8.957 | 1.00 | 20.00 | B | C |
| ATOM | 6690 | CD | GLU H | 463 | −26.341 | −11.973 | −9.490 | 1.00 | 20.00 | B | C |
| ATOM | 6691 | OE1 | GLU H | 463 | −26.167 | −11.917 | −10.729 | 1.00 | 20.00 | B | O |
| ATOM | 6692 | OE2 | GLU H | 463 | −25.399 | −11.890 | −8.672 | 1.00 | 20.00 | B | O |
| ATOM | 6693 | C | GLU H | 463 | −31.208 | −12.245 | −10.629 | 1.00 | 116.39 | B | C |
| ATOM | 6694 | O | GLU H | 463 | −31.442 | −13.153 | −11.426 | 1.00 | 118.70 | B | O |
| ATOM | 6695 | N | GLY H | 464 | −31.759 | −11.039 | −10.722 | 1.00 | 127.70 | B | N |
| ATOM | 6696 | CA | GLY H | 464 | −32.671 | −10.693 | −11.806 | 1.00 | 143.17 | B | C |
| ATOM | 6697 | C | GLY H | 464 | −31.941 | −10.347 | −13.089 | 1.00 | 149.77 | B | C |
| ATOM | 6698 | O | GLY H | 464 | −30.719 | −10.197 | −13.098 | 1.00 | 155.06 | B | O |
| ATOM | 6699 | N | LYS H | 465 | −32.694 | −10.223 | −14.178 | 1.00 | 153.73 | B | N |
| ATOM | 6700 | CA | LYS H | 465 | −32.115 | −9.929 | −15.484 | 1.00 | 153.69 | B | C |
| ATOM | 6701 | CB | LYS H | 465 | −31.275 | −8.651 | −15.424 | 1.00 | 20.00 | B | C |
| ATOM | 6702 | CG | LYS H | 465 | −32.085 | −7.385 | −15.193 | 1.00 | 20.00 | B | C |
| ATOM | 6703 | CD | LYS H | 465 | −31.186 | −6.163 | −15.106 | 1.00 | 20.00 | B | C |
| ATOM | 6704 | CE | LYS H | 465 | −31.995 | −4.898 | −14.868 | 1.00 | 20.00 | B | C |
| ATOM | 6705 | NZ | LYS H | 465 | −31.127 | −3.692 | −14.776 | 1.00 | 20.00 | B | N |
| ATOM | 6706 | C | LYS H | 465 | −31.267 | −11.091 | −15.990 | 1.00 | 155.37 | B | C |
| ATOM | 6707 | O | LYS H | 465 | −31.276 | −11.407 | −17.180 | 1.00 | 155.20 | B | O |
| ATOM | 6708 | N | GLU H | 472 | −30.692 | −9.899 | −23.132 | 1.00 | 145.47 | B | N |
| ATOM | 6709 | CA | GLU H | 472 | −30.214 | −9.535 | −24.461 | 1.00 | 151.12 | B | C |
| ATOM | 6710 | CB | GLU H | 472 | −30.931 | −8.281 | −24.964 | 1.00 | 20.00 | B | C |
| ATOM | 6711 | CG | GLU H | 472 | −32.419 | −8.472 | −25.209 | 1.00 | 20.00 | B | C |
| ATOM | 6712 | CD | GLU H | 472 | −33.106 | −7.192 | −25.643 | 1.00 | 20.00 | B | C |
| ATOM | 6713 | OE1 | GLU H | 472 | −32.477 | −6.117 | −25.554 | 1.00 | 20.00 | B | O |
| ATOM | 6714 | OE2 | GLU H | 472 | −34.279 | −7.260 | −26.070 | 1.00 | 20.00 | B | O |
| ATOM | 6715 | C | GLU H | 472 | −28.705 | −9.310 | −24.462 | 1.00 | 153.35 | B | C |
| ATOM | 6716 | O | GLU H | 472 | −28.208 | −8.395 | −23.805 | 1.00 | 159.41 | B | O |
| ATOM | 6717 | N | PRO H | 473 | −27.971 | −10.164 | −25.188 | 1.00 | 150.51 | B | N |
| ATOM | 6718 | CA | PRO H | 473 | −26.535 | −10.028 | −25.339 | 1.00 | 141.97 | B | C |
| ATOM | 6719 | CB | PRO H | 473 | −26.035 | −11.435 | −25.011 | 1.00 | 20.00 | B | C |
| ATOM | 6720 | CG | PRO H | 473 | −27.175 | −12.350 | −25.428 | 1.00 | 20.00 | B | C |
| ATOM | 6721 | CD | PRO H | 473 | −28.424 | −11.506 | −25.583 | 1.00 | 20.00 | B | C |
| ATOM | 6722 | C | PRO H | 473 | −26.193 | −9.688 | −26.782 | 1.00 | 135.14 | B | C |
| ATOM | 6723 | O | PRO H | 473 | −26.968 | −9.997 | −27.687 | 1.00 | 144.93 | B | O |
| ATOM | 6724 | N | ILE H | 474 | −25.024 | −9.096 | −27.001 | 1.00 | 118.07 | B | N |
| ATOM | 6725 | CA | ILE H | 474 | −24.617 | −8.700 | −28.344 | 1.00 | 105.14 | B | C |
| ATOM | 6726 | CB | ILE H | 474 | −25.549 | −7.614 | −28.919 | 1.00 | 20.00 | B | C |
| ATOM | 6727 | CG1 | ILE H | 474 | −26.957 | −8.174 | −29.133 | 1.00 | 20.00 | B | C |
| ATOM | 6728 | CD1 | ILE H | 474 | −28.021 | −7.109 | −29.293 | 1.00 | 20.00 | B | C |
| ATOM | 6729 | CG2 | ILE H | 474 | −24.988 | −7.064 | −30.222 | 1.00 | 20.00 | B | C |
| ATOM | 6730 | C | ILE H | 474 | −23.184 | −8.180 | −28.352 | 1.00 | 104.75 | B | C |
| ATOM | 6731 | O | ILE H | 474 | −22.938 | −7.013 | −28.046 | 1.00 | 111.83 | B | O |
| ATOM | 6732 | N | ILE H | 475 | −22.242 | −9.045 | −28.716 | 1.00 | 94.65 | B | N |
| ATOM | 6733 | CA | ILE H | 475 | −20.826 | −8.732 | −28.565 | 1.00 | 79.88 | B | C |
| ATOM | 6734 | CB | ILE H | 475 | −20.175 | −9.577 | −27.453 | 1.00 | 20.00 | B | C |
| ATOM | 6735 | CG1 | ILE H | 475 | −20.674 | −9.120 | −26.079 | 1.00 | 20.00 | B | C |
| ATOM | 6736 | CD1 | ILE H | 475 | −20.434 | −10.125 | −24.972 | 1.00 | 20.00 | B | C |
| ATOM | 6737 | CG2 | ILE H | 475 | −18.658 | −9.507 | −27.545 | 1.00 | 20.00 | B | C |
| ATOM | 6738 | C | ILE H | 475 | −20.051 | −8.909 | −29.868 | 1.00 | 79.52 | B | C |
| ATOM | 6739 | O | ILE H | 475 | −19.808 | −10.032 | −30.310 | 1.00 | 82.70 | B | O |
| ATOM | 6740 | N | ASN H | 476 | −19.589 | −7.793 | −30.425 | 1.00 | 69.64 | B | N |
| ATOM | 6741 | CA | ASN H | 476 | −18.756 | −7.812 | −31.623 | 1.00 | 68.88 | B | C |
| ATOM | 6742 | CB | ASN H | 476 | −18.880 | −6.487 | −32.381 | 1.00 | 20.00 | B | C |
| ATOM | 6743 | CG | ASN H | 476 | −20.247 | −6.299 | −33.011 | 1.00 | 20.00 | B | C |
| ATOM | 6744 | OD1 | ASN H | 476 | −20.954 | −7.268 | −33.288 | 1.00 | 20.00 | B | O |
| ATOM | 6745 | ND2 | ASN H | 476 | −20.618 | −5.048 | −33.257 | 1.00 | 20.00 | B | N |
| ATOM | 6746 | C | ASN H | 476 | −17.290 | −8.097 | −31.306 | 1.00 | 67.13 | B | C |
| ATOM | 6747 | O | ASN H | 476 | −16.718 | −7.507 | −30.389 | 1.00 | 73.10 | B | O |
| ATOM | 6748 | N | PHE H | 477 | −16.677 | −8.975 | −32.096 | 1.00 | 64.95 | B | N |
| ATOM | 6749 | CA | PHE H | 477 | −15.288 | −9.380 | −31.879 | 1.00 | 68.13 | B | C |
| ATOM | 6750 | CB | PHE H | 477 | −14.918 | −10.567 | −32.776 | 1.00 | 20.00 | B | C |
| ATOM | 6751 | CG | PHE H | 477 | −15.678 | −11.822 | −32.466 | 1.00 | 20.00 | B | C |
| ATOM | 6752 | CD1 | PHE H | 477 | −15.266 | −12.666 | −31.448 | 1.00 | 20.00 | B | C |
| ATOM | 6753 | CE1 | PHE H | 477 | −15.965 | −13.820 | −31.162 | 1.00 | 20.00 | B | C |
| ATOM | 6754 | CZ | PHE H | 477 | −17.069 | −14.158 | −31.915 | 1.00 | 20.00 | B | C |
| ATOM | 6755 | CE2 | PHE H | 477 | −17.473 | −13.339 | −32.949 | 1.00 | 20.00 | B | C |
| ATOM | 6756 | CD2 | PHE H | 477 | −16.778 | −12.181 | −33.222 | 1.00 | 20.00 | B | C |
| ATOM | 6757 | C | PHE H | 477 | −14.307 | −8.232 | −32.117 | 1.00 | 65.57 | B | C |
| ATOM | 6758 | O | PHE H | 477 | −13.094 | −8.412 | −31.999 | 1.00 | 49.83 | B | O |
| ATOM | 6759 | N | TYR H | 478 | −14.816 | −7.113 | −32.617 | 1.00 | 51.97 | B | N |
| ATOM | 6760 | CA | TYR H | 478 | −13.996 | −5.920 | −32.779 | 1.00 | 52.01 | B | C |
| ATOM | 6761 | CB | TYR H | 478 | −14.573 | −5.017 | −33.870 | 1.00 | 20.00 | B | C |
| ATOM | 6762 | CG | TYR H | 478 | −14.579 | −5.653 | −35.247 | 1.00 | 20.00 | B | C |
| ATOM | 6763 | CD1 | TYR H | 478 | −13.458 | −5.593 | −36.069 | 1.00 | 20.00 | B | C |
| ATOM | 6764 | CE1 | TYR H | 478 | −13.453 | −6.195 | −37.316 | 1.00 | 20.00 | B | C |
| ATOM | 6765 | CZ | TYR H | 478 | −14.585 | −6.849 | −37.766 | 1.00 | 20.00 | B | C |
| ATOM | 6766 | OH | TYR H | 478 | −14.597 | −7.434 | −39.013 | 1.00 | 20.00 | B | O |
| ATOM | 6767 | CE2 | TYR H | 478 | −15.689 | −6.967 | −36.948 | 1.00 | 20.00 | B | C |
| ATOM | 6768 | CD2 | TYR H | 478 | −15.687 | −6.360 | −35.703 | 1.00 | 20.00 | B | C |
| ATOM | 6769 | C | TYR H | 478 | −13.909 | −5.178 | −31.454 | 1.00 | 66.63 | B | C |
| ATOM | 6770 | O | TYR H | 478 | −13.401 | −4.059 | −31.383 | 1.00 | 63.45 | B | O |
| ATOM | 6771 | N | ASP H | 479 | −14.391 | −5.828 | −30.401 | 1.00 | 65.34 | B | N |
| ATOM | 6772 | CA | ASP H | 479 | −14.256 | −5.311 | −29.049 | 1.00 | 65.32 | B | C |
| ATOM | 6773 | CB | ASP H | 479 | −15.579 | −5.450 | −28.296 | 1.00 | 20.00 | B | C |
| ATOM | 6774 | CG | ASP H | 479 | −16.664 | −4.552 | −28.856 | 1.00 | 20.00 | B | C |
| ATOM | 6775 | OD1 | ASP H | 479 | −16.327 | −3.593 | −29.582 | 1.00 | 20.00 | B | O |
| ATOM | 6776 | OD2 | ASP H | 479 | −17.854 | −4.799 | −28.568 | 1.00 | 20.00 | B | O |
| ATOM | 6777 | C | ASP H | 479 | −13.144 | −6.036 | −28.300 | 1.00 | 64.83 | B | C |
| ATOM | 6778 | O | ASP H | 479 | −12.738 | −5.609 | −27.220 | 1.00 | 60.42 | B | O |
| ATOM | 6779 | N | PRO H | 480 | −12.610 | −7.108 | −28.903 | 1.00 | 47.60 | B | N |
| ATOM | 6780 | CA | PRO H | 480 | −11.568 | −7.902 | −28.282 | 1.00 | 46.52 | B | C |
| ATOM | 6781 | CB | PRO H | 480 | −11.905 | −9.318 | −28.742 | 1.00 | 20.00 | B | C |
| ATOM | 6782 | CG | PRO H | 480 | −12.596 | −9.131 | −30.072 | 1.00 | 20.00 | B | C |
| ATOM | 6783 | CD | PRO H | 480 | −13.021 | −7.682 | −30.193 | 1.00 | 20.00 | B | C |
| ATOM | 6784 | C | PRO H | 480 | −10.199 | −7.497 | −28.809 | 1.00 | 52.36 | B | C |
| ATOM | 6785 | O | PRO H | 480 | −9.736 | −8.035 | −29.815 | 1.00 | 64.15 | B | O |
| ATOM | 6786 | N | LEU H | 481 | −9.618 | −6.472 | −28.197 | 1.00 | 62.90 | B | N |
| ATOM | 6787 | CA | LEU H | 481 | −8.285 | −6.015 | −28.562 | 1.00 | 54.24 | B | C |
| ATOM | 6788 | CB | LEU H | 481 | −8.322 | −4.544 | −28.982 | 1.00 | 20.00 | B | C |
| ATOM | 6789 | CG | LEU H | 481 | −9.202 | −4.212 | −30.186 | 1.00 | 20.00 | B | C |
| ATOM | 6790 | CD1 | LEU H | 481 | −9.255 | −2.706 | −30.408 | 1.00 | 20.00 | B | C |
| ATOM | 6791 | CD2 | LEU H | 481 | −8.698 | −4.931 | −31.425 | 1.00 | 20.00 | B | C |
| ATOM | 6792 | C | LEU H | 481 | −7.315 | −6.203 | −27.402 | 1.00 | 59.81 | B | C |
| ATOM | 6793 | O | LEU H | 481 | −7.682 | −6.724 | −26.348 | 1.00 | 64.98 | B | O |
| ATOM | 6794 | N | VAL H | 482 | −6.058 | −5.840 | −27.628 | 1.00 | 59.66 | B | N |
| ATOM | 6795 | CA | VAL H | 482 | −5.084 | −5.781 | −26.549 | 1.00 | 49.67 | B | C |
| ATOM | 6796 | CB | VAL H | 482 | −3.786 | −6.531 | −26.911 | 1.00 | 20.00 | B | C |
| ATOM | 6797 | CG1 | VAL H | 482 | −2.811 | −6.492 | −25.742 | 1.00 | 20.00 | B | C |
| ATOM | 6798 | CG2 | VAL H | 482 | −4.097 | −7.973 | −27.288 | 1.00 | 20.00 | B | C |
| ATOM | 6799 | C | VAL H | 482 | −4.762 | −4.337 | −26.191 | 1.00 | 53.03 | B | C |
| ATOM | 6800 | O | VAL H | 482 | −4.706 | −3.472 | −27.064 | 1.00 | 46.27 | B | O |
| ATOM | 6801 | N | PHE H | 483 | −4.641 | −4.070 | −24.894 | 1.00 | 61.61 | B | N |
| ATOM | 6802 | CA | PHE H | 483 | −4.311 | −2.732 | −24.415 | 1.00 | 65.03 | B | C |
| ATOM | 6803 | CB | PHE H | 483 | −5.338 | −2.259 | −23.391 | 1.00 | 20.00 | B | C |
| ATOM | 6804 | CG | PHE H | 483 | −6.714 | −2.064 | −23.966 | 1.00 | 20.00 | B | C |
| ATOM | 6805 | CD1 | PHE H | 483 | −7.021 | −0.928 | −24.695 | 1.00 | 20.00 | B | C |
| ATOM | 6806 | CE1 | PHE H | 483 | −8.294 | −0.739 | −25.209 | 1.00 | 20.00 | B | C |
| ATOM | 6807 | CZ | PHE H | 483 | −9.246 | −1.731 | −25.077 | 1.00 | 20.00 | B | C |
| ATOM | 6808 | CE2 | PHE H | 483 | −8.937 | −2.888 | −24.397 | 1.00 | 20.00 | B | C |
| ATOM | 6809 | CD2 | PHE H | 483 | −7.667 | −3.065 | −23.872 | 1.00 | 20.00 | B | C |
| ATOM | 6810 | C | PHE H | 483 | −2.897 | −2.662 | −23.852 | 1.00 | 71.00 | B | C |
| ATOM | 6811 | O | PHE H | 483 | −2.704 | −2.417 | −22.659 | 1.00 | 100.44 | B | O |
| ATOM | 6812 | N | PRO H | 484 | −1.909 | −2.972 | −24.705 | 1.00 | 54.44 | B | N |
| ATOM | 6813 | CA | PRO H | 484 | −0.512 | −3.000 | −24.329 | 1.00 | 47.46 | B | C |
| ATOM | 6814 | CB | PRO H | 484 | −0.179 | −4.492 | −24.438 | 1.00 | 20.00 | B | C |
| ATOM | 6815 | CG | PRO H | 484 | −1.142 | −5.021 | −25.513 | 1.00 | 20.00 | B | C |
| ATOM | 6816 | CD | PRO H | 484 | −2.157 | −3.926 | −25.796 | 1.00 | 20.00 | B | C |
| ATOM | 6817 | C | PRO H | 484 | 0.323 | −2.225 | −25.346 | 1.00 | 41.56 | B | C |
| ATOM | 6818 | O | PRO H | 484 | 0.278 | −2.527 | −26.539 | 1.00 | 46.53 | B | O |
| ATOM | 6819 | N | SER H | 485 | 1.033 | −1.202 | −24.883 | 1.00 | 46.30 | B | N |
| ATOM | 6820 | CA | SER H | 485 | 1.824 | −0.355 | −25.768 | 1.00 | 49.30 | B | C |
| ATOM | 6821 | CB | SER H | 485 | 3.010 | 0.252 | −25.015 | 1.00 | 20.00 | B | C |
| ATOM | 6822 | OG | SER H | 485 | 3.864 | −0.754 | −24.502 | 1.00 | 20.00 | B | O |
| ATOM | 6823 | C | SER H | 485 | 2.307 | −1.112 | −27.003 | 1.00 | 41.24 | B | C |
| ATOM | 6824 | O | SER H | 485 | 1.964 | −0.755 | −28.131 | 1.00 | 46.93 | B | O |
| ATOM | 6825 | N | ASP H | 486 | 3.108 | −2.150 | −26.785 | 1.00 | 49.48 | B | N |
| ATOM | 6826 | CA | ASP H | 486 | 3.700 | −2.909 | −27.881 | 1.00 | 37.44 | B | C |
| ATOM | 6827 | CB | ASP H | 486 | 4.552 | −4.054 | −27.333 | 1.00 | 20.00 | B | C |
| ATOM | 6828 | CG | ASP H | 486 | 5.885 | −3.576 | −26.790 | 1.00 | 20.00 | B | C |
| ATOM | 6829 | OD1 | ASP H | 486 | 6.284 | −2.437 | −27.115 | 1.00 | 20.00 | B | O |
| ATOM | 6830 | OD2 | ASP H | 486 | 6.556 | −4.355 | −26.080 | 1.00 | 20.00 | B | O |
| ATOM | 6831 | C | ASP H | 486 | 2.628 | −3.456 | −28.818 | 1.00 | 45.66 | B | C |
| ATOM | 6832 | O | ASP H | 486 | 2.683 | −3.252 | −30.034 | 1.00 | 39.86 | B | O |
| ATOM | 6833 | N | GLU H | 487 | 1.627 | −4.107 | −28.238 | 1.00 | 49.20 | B | N |
| ATOM | 6834 | CA | GLU H | 487 | 0.525 | −4.652 | −29.013 | 1.00 | 45.70 | B | C |
| ATOM | 6835 | CB | GLU H | 487 | −0.444 | −5.410 | −28.105 | 1.00 | 20.00 | B | C |
| ATOM | 6836 | CG | GLU H | 487 | 0.156 | −6.664 | −27.483 | 1.00 | 20.00 | B | C |
| ATOM | 6837 | CD | GLU H | 487 | −0.814 | −7.393 | −26.576 | 1.00 | 20.00 | B | C |
| ATOM | 6838 | OE1 | GLU H | 487 | −1.852 | −6.804 | −26.214 | 1.00 | 20.00 | B | O |
| ATOM | 6839 | OE2 | GLU H | 487 | −0.514 | −8.540 | −26.183 | 1.00 | 20.00 | B | O |
| ATOM | 6840 | C | GLU H | 487 | −0.203 | −3.556 | −29.783 | 1.00 | 41.48 | B | C |
| ATOM | 6841 | O | GLU H | 487 | −0.411 | −3.674 | −30.989 | 1.00 | 48.92 | B | O |
| ATOM | 6842 | N | PHE H | 488 | −0.486 | −2.447 | −29.110 | 1.00 | 42.16 | B | N |
| ATOM | 6843 | CA | PHE H | 488 | −1.183 | −1.339 | −29.747 | 1.00 | 39.33 | B | C |
| ATOM | 6844 | CB | PHE H | 488 | −1.438 | −0.219 | −28.745 | 1.00 | 20.00 | B | C |
| ATOM | 6845 | CG | PHE H | 488 | −2.369 | −0.602 | −27.638 | 1.00 | 20.00 | B | C |
| ATOM | 6846 | CD1 | PHE H | 488 | −3.739 | −0.595 | −27.833 | 1.00 | 20.00 | B | C |
| ATOM | 6847 | CE1 | PHE H | 488 | −4.602 | −0.873 | −26.789 | 1.00 | 20.00 | B | C |
| ATOM | 6848 | CZ | PHE H | 488 | −4.095 | −1.193 | −25.546 | 1.00 | 20.00 | B | C |
| ATOM | 6849 | CE2 | PHE H | 488 | −2.731 | −1.192 | −25.340 | 1.00 | 20.00 | B | C |
| ATOM | 6850 | CD2 | PHE H | 488 | −1.876 | −0.912 | −26.385 | 1.00 | 20.00 | B | C |
| ATOM | 6851 | C | PHE H | 488 | −0.401 | −0.804 | −30.939 | 1.00 | 43.06 | B | C |
| ATOM | 6852 | O | PHE H | 488 | −0.977 | −0.505 | −31.984 | 1.00 | 61.71 | B | O |
| ATOM | 6853 | N | ASP H | 489 | 0.915 | −0.700 | −30.782 | 1.00 | 41.95 | B | N |
| ATOM | 6854 | CA | ASP H | 489 | 1.766 | −0.104 | −31.808 | 1.00 | 42.75 | B | C |
| ATOM | 6855 | CB | ASP H | 489 | 3.172 | 0.134 | −31.262 | 1.00 | 20.00 | B | C |
| ATOM | 6856 | CG | ASP H | 489 | 3.235 | 1.317 | −30.327 | 1.00 | 20.00 | B | C |
| ATOM | 6857 | OD1 | ASP H | 489 | 2.256 | 2.089 | −30.288 | 1.00 | 20.00 | B | O |
| ATOM | 6858 | OD2 | ASP H | 489 | 4.252 | 1.462 | −29.617 | 1.00 | 20.00 | B | O |
| ATOM | 6859 | C | ASP H | 489 | 1.837 | −0.980 | −33.054 | 1.00 | 45.61 | B | C |
| ATOM | 6860 | O | ASP H | 489 | 2.110 | −0.495 | −34.154 | 1.00 | 54.39 | B | O |
| ATOM | 6861 | N | ALA H | 490 | 1.702 | −2.287 | −32.853 | 1.00 | 42.89 | B | N |
| ATOM | 6862 | CA | ALA H | 490 | 1.705 | −3.238 | −33.957 | 1.00 | 38.40 | B | C |
| ATOM | 6863 | CB | ALA H | 490 | 1.928 | −4.653 | −33.436 | 1.00 | 20.00 | B | C |
| ATOM | 6864 | C | ALA H | 490 | 0.413 | −3.160 | −34.770 | 1.00 | 39.67 | B | C |
| ATOM | 6865 | O | ALA H | 490 | 0.443 | −3.194 | −35.998 | 1.00 | 56.80 | B | O |
| ATOM | 6866 | N | SER H | 491 | −0.717 | −3.050 | −34.080 | 1.00 | 40.38 | B | N |
| ATOM | 6867 | CA | SER H | 491 | −2.003 | −2.833 | −34.737 | 1.00 | 41.77 | B | C |
| ATOM | 6868 | CB | SER H | 491 | −3.155 | −2.963 | −33.731 | 1.00 | 20.00 | B | C |
| ATOM | 6869 | OG | SER H | 491 | −3.173 | −1.878 | −32.817 | 1.00 | 20.00 | B | O |
| ATOM | 6870 | C | SER H | 491 | −2.056 | −1.474 | −35.432 | 1.00 | 39.01 | B | C |
| ATOM | 6871 | O | SER H | 491 | −2.701 | −1.325 | −36.472 | 1.00 | 36.91 | B | O |
| ATOM | 6872 | N | ILE H | 492 | −1.440 | −0.470 | −34.812 | 1.00 | 38.11 | B | N |
| ATOM | 6873 | CA | ILE H | 492 | −1.342 | 0.863 | −35.402 | 1.00 | 41.21 | B | C |
| ATOM | 6874 | CB | ILE H | 492 | −0.651 | 1.856 | −34.446 | 1.00 | 20.00 | B | C |
| ATOM | 6875 | CG1 | ILE H | 492 | −1.506 | 2.097 | −33.203 | 1.00 | 20.00 | B | C |
| ATOM | 6876 | CD1 | ILE H | 492 | −0.883 | 3.081 | −32.232 | 1.00 | 20.00 | B | C |
| ATOM | 6877 | CG2 | ILE H | 492 | −0.363 | 3.173 | −35.150 | 1.00 | 20.00 | B | C |
| ATOM | 6878 | C | ILE H | 492 | −0.561 | 0.826 | −36.714 | 1.00 | 39.22 | B | C |
| ATOM | 6879 | O | ILE H | 492 | −0.943 | 1.466 | −37.696 | 1.00 | 47.54 | B | O |
| ATOM | 6880 | N | SER H | 493 | 0.535 | 0.073 | −36.718 | 1.00 | 41.64 | B | N |
| ATOM | 6881 | CA | SER H | 493 | 1.307 | −0.161 | −37.933 | 1.00 | 35.15 | B | C |
| ATOM | 6882 | CB | SER H | 493 | 2.587 | −0.944 | −37.618 | 1.00 | 20.00 | B | C |
| ATOM | 6883 | OG | SER H | 493 | 2.296 | −2.250 | −37.147 | 1.00 | 20.00 | B | O |
| ATOM | 6884 | C | SER H | 493 | 0.486 | −0.880 | −39.011 | 1.00 | 47.90 | B | C |
| ATOM | 6885 | O | SER H | 493 | 0.565 | −0.538 | −40.191 | 1.00 | 50.94 | B | O |
| ATOM | 6886 | N | GLN H | 494 | −0.321 | −1.855 | −38.596 | 1.00 | 42.51 | B | N |
| ATOM | 6887 | CA | GLN H | 494 | −1.175 | −2.611 | −39.517 | 1.00 | 41.96 | B | C |
| ATOM | 6888 | CB | GLN H | 494 | −1.822 | −3.801 | −38.795 | 1.00 | 20.00 | B | C |
| ATOM | 6889 | CG | GLN H | 494 | −0.843 | −4.905 | −38.423 | 1.00 | 20.00 | B | C |
| ATOM | 6890 | CD | GLN H | 494 | −1.489 | −6.019 | −37.630 | 1.00 | 20.00 | B | C |
| ATOM | 6891 | OE1 | GLN H | 494 | −2.536 | −5.830 | −37.011 | 1.00 | 20.00 | B | O |
| ATOM | 6892 | NE2 | GLN H | 494 | −0.893 | −7.205 | −37.684 | 1.00 | 20.00 | B | N |
| ATOM | 6893 | C | GLN H | 494 | −2.254 | −1.721 | −40.130 | 1.00 | 33.86 | B | C |
| ATOM | 6894 | O | GLN H | 494 | −2.479 | −1.743 | −41.340 | 1.00 | 54.89 | B | O |
| ATOM | 6895 | N | VAL H | 495 | −2.973 | −1.005 | −39.272 | 1.00 | 34.99 | B | N |
| ATOM | 6896 | CA | VAL H | 495 | −3.955 | −0.025 | −39.718 | 1.00 | 38.42 | B | C |
| ATOM | 6897 | CB | VAL H | 495 | −4.485 | 0.807 | −38.536 | 1.00 | 20.00 | B | C |
| ATOM | 6898 | CG1 | VAL H | 495 | −5.501 | 1.828 | −39.016 | 1.00 | 20.00 | B | C |
| ATOM | 6899 | CG2 | VAL H | 495 | −5.088 | −0.101 | −37.479 | 1.00 | 20.00 | B | C |
| ATOM | 6900 | C | VAL H | 495 | −3.344 | 0.912 | −40.751 | 1.00 | 42.74 | B | C |
| ATOM | 6901 | O | VAL H | 495 | −3.876 | 1.069 | −41.850 | 1.00 | 44.00 | B | O |
| ATOM | 6902 | N | ASN H | 496 | −2.171 | 1.447 | −40.434 | 1.00 | 42.45 | B | N |
| ATOM | 6903 | CA | ASN H | 496 | −1.500 | 2.382 | −41.323 | 1.00 | 43.01 | B | C |
| ATOM | 6904 | CB | ASN H | 496 | −0.202 | 2.880 | −40.692 | 1.00 | 20.00 | B | C |
| ATOM | 6905 | CG | ASN H | 496 | −0.446 | 3.770 | −39.497 | 1.00 | 20.00 | B | C |
| ATOM | 6906 | OD1 | ASN H | 496 | −1.517 | 4.360 | −39.358 | 1.00 | 20.00 | B | O |
| ATOM | 6907 | ND2 | ASN H | 496 | 0.537 | 3.849 | −38.608 | 1.00 | 20.00 | B | N |
| ATOM | 6908 | C | ASN H | 496 | −1.227 | 1.780 | −42.695 | 1.00 | 43.13 | B | C |
| ATOM | 6909 | O | ASN H | 496 | −1.502 | 2.404 | −43.720 | 1.00 | 54.76 | B | O |
| ATOM | 6910 | N | GLU H | 497 | −0.737 | 0.544 | −42.708 | 1.00 | 42.08 | B | N |
| ATOM | 6911 | CA | GLU H | 497 | −0.453 | −0.150 | −43.957 | 1.00 | 43.13 | B | C |
| ATOM | 6912 | CB | GLU H | 497 | 0.151 | −1.525 | −43.678 | 1.00 | 20.00 | B | C |
| ATOM | 6913 | CG | GLU H | 497 | 1.651 | −1.515 | −43.476 | 1.00 | 20.00 | B | C |
| ATOM | 6914 | CD | GLU H | 497 | 2.193 | −2.884 | −43.108 | 1.00 | 20.00 | B | C |
| ATOM | 6915 | OE1 | GLU H | 497 | 1.402 | −3.851 | −43.058 | 1.00 | 20.00 | B | O |
| ATOM | 6916 | OE2 | GLU H | 497 | 3.419 | −3.001 | −42.899 | 1.00 | 20.00 | B | O |
| ATOM | 6917 | C | GLU H | 497 | −1.724 | −0.306 | −44.781 | 1.00 | 49.77 | B | C |
| ATOM | 6918 | O | GLU H | 497 | −1.680 | −0.352 | −46.011 | 1.00 | 42.88 | B | O |
| ATOM | 6919 | N | LYS H | 498 | −2.846 | −0.477 | −44.088 | 1.00 | 41.82 | B | N |
| ATOM | 6920 | CA | LYS H | 498 | −4.141 | −0.621 | −44.741 | 1.00 | 53.60 | B | C |
| ATOM | 6921 | CB | LYS H | 498 | −5.174 | −1.179 | −43.761 | 1.00 | 20.00 | B | C |
| ATOM | 6922 | CG | LYS H | 498 | −4.967 | −2.641 | −43.403 | 1.00 | 20.00 | B | C |
| ATOM | 6923 | CD | LYS H | 498 | −6.078 | −3.148 | −42.491 | 1.00 | 20.00 | B | C |
| ATOM | 6924 | CE | LYS H | 498 | −5.896 | −4.624 | −42.160 | 1.00 | 20.00 | B | C |
| ATOM | 6925 | NZ | LYS H | 498 | −6.862 | −5.090 | −41.127 | 1.00 | 20.00 | B | N |
| ATOM | 6926 | C | LYS H | 498 | −4.622 | 0.716 | −45.298 | 1.00 | 55.29 | B | C |
| ATOM | 6927 | O | LYS H | 498 | −5.273 | 0.766 | −46.340 | 1.00 | 61.06 | B | O |
| ATOM | 6928 | N | ILE H | 499 | −4.359 | 1.789 | −44.558 | 1.00 | 52.22 | B | N |
| ATOM | 6929 | CA | ILE H | 499 | −4.606 | 3.140 | −45.052 | 1.00 | 48.26 | B | C |
| ATOM | 6930 | CB | ILE H | 499 | −4.213 | 4.205 | −44.012 | 1.00 | 20.00 | B | C |
| ATOM | 6931 | CG1 | ILE H | 499 | −5.137 | 4.139 | −42.797 | 1.00 | 20.00 | B | C |
| ATOM | 6932 | CD1 | ILE H | 499 | −4.719 | 5.058 | −41.669 | 1.00 | 20.00 | B | C |
| ATOM | 6933 | CG2 | ILE H | 499 | −4.246 | 5.594 | −44.631 | 1.00 | 20.00 | B | C |
| ATOM | 6934 | C | ILE H | 499 | −3.809 | 3.400 | −46.323 | 1.00 | 49.07 | B | C |
| ATOM | 6935 | O | ILE H | 499 | −4.273 | 4.090 | −47.232 | 1.00 | 58.45 | B | O |
| ATOM | 6936 | N | ASN H | 500 | −2.600 | 2.849 | −46.371 | 1.00 | 51.02 | B | N |
| ATOM | 6937 | CA | ASN H | 500 | −1.718 | 3.000 | −47.524 | 1.00 | 52.06 | B | C |
| ATOM | 6938 | CB | ASN H | 500 | −0.284 | 2.663 | −47.120 | 1.00 | 20.00 | B | C |
| ATOM | 6939 | CG | ASN H | 500 | 0.707 | 2.898 | −48.233 | 1.00 | 20.00 | B | C |
| ATOM | 6940 | OD1 | ASN H | 500 | 0.607 | 3.875 | −48.973 | 1.00 | 20.00 | B | O |
| ATOM | 6941 | ND2 | ASN H | 500 | 1.740 | 2.071 | −48.275 | 1.00 | 20.00 | B | N |
| ATOM | 6942 | C | ASN H | 500 | −2.151 | 2.149 | −48.719 | 1.00 | 48.31 | B | C |
| ATOM | 6943 | O | ASN H | 500 | −1.841 | 2.469 | −49.867 | 1.00 | 56.89 | B | O |
| ATOM | 6944 | N | GLN H | 501 | −2.872 | 1.067 | −48.438 | 1.00 | 55.87 | B | N |
| ATOM | 6945 | CA | GLN H | 501 | −3.511 | 0.269 | −49.480 | 1.00 | 53.55 | B | C |
| ATOM | 6946 | CB | GLN H | 501 | −3.879 | −1.116 | −48.940 | 1.00 | 20.00 | B | C |
| ATOM | 6947 | CG | GLN H | 501 | −2.685 | −1.963 | −48.513 | 1.00 | 20.00 | B | C |
| ATOM | 6948 | CD | GLN H | 501 | −3.093 | −3.333 | −47.996 | 1.00 | 20.00 | B | C |
| ATOM | 6949 | OE1 | GLN H | 501 | −4.169 | −3.495 | −47.420 | 1.00 | 20.00 | B | O |
| ATOM | 6950 | NE2 | GLN H | 501 | −2.215 | −4.317 | −48.162 | 1.00 | 20.00 | B | N |
| ATOM | 6951 | C | GLN H | 501 | −4.755 | 0.964 | −50.023 | 1.00 | 45.67 | B | C |
| ATOM | 6952 | O | GLN H | 501 | −4.984 | 0.994 | −51.232 | 1.00 | 58.31 | B | O |
| ATOM | 6953 | N | SER H | 502 | −5.525 | 1.570 | −49.124 | 1.00 | 43.44 | B | N |
| ATOM | 6954 | CA | SER H | 502 | −6.702 | 2.345 | −49.508 | 1.00 | 45.40 | B | C |
| ATOM | 6955 | CB | SER H | 502 | −7.471 | 2.800 | −48.264 | 1.00 | 20.00 | B | C |
| ATOM | 6956 | OG | SER H | 502 | −6.684 | 3.664 | −47.464 | 1.00 | 20.00 | B | O |
| ATOM | 6957 | C | SER H | 502 | −6.319 | 3.555 | −50.358 | 1.00 | 55.15 | B | C |
| ATOM | 6958 | O | SER H | 502 | −7.049 | 3.943 | −51.270 | 1.00 | 52.57 | B | O |
| ATOM | 6959 | N | LEU H | 503 | −5.160 | 4.135 | −50.060 | 1.00 | 55.35 | B | N |
| ATOM | 6960 | CA | LEU H | 503 | −4.662 | 5.286 | −50.805 | 1.00 | 52.12 | B | C |
| ATOM | 6961 | CB | LEU H | 503 | −3.529 | 5.973 | −50.037 | 1.00 | 20.00 | B | C |
| ATOM | 6962 | CG | LEU H | 503 | −3.896 | 6.598 | −48.688 | 1.00 | 20.00 | B | C |
| ATOM | 6963 | CD1 | LEU H | 503 | −2.646 | 7.032 | −47.948 | 1.00 | 20.00 | B | C |
| ATOM | 6964 | CD2 | LEU H | 503 | −4.847 | 7.769 | −48.868 | 1.00 | 20.00 | B | C |
| ATOM | 6965 | C | LEU H | 503 | −4.187 | 4.882 | −52.197 | 1.00 | 55.74 | B | C |
| ATOM | 6966 | O | LEU H | 503 | −4.339 | 5.636 | −53.159 | 1.00 | 57.75 | B | O |
| ATOM | 6967 | N | ALA H | 504 | −3.608 | 3.691 | −52.295 | 1.00 | 57.22 | B | N |
| ATOM | 6968 | CA | ALA H | 504 | −3.204 | 3.150 | −53.584 | 1.00 | 59.12 | B | C |
| ATOM | 6969 | CB | ALA H | 504 | −2.319 | 1.930 | −53.394 | 1.00 | 20.00 | B | C |
| ATOM | 6970 | C | ALA H | 504 | −4.423 | 2.802 | −54.427 | 1.00 | 58.10 | B | C |
| ATOM | 6971 | O | ALA H | 504 | −4.488 | 3.145 | −55.608 | 1.00 | 63.64 | B | O |
| ATOM | 6972 | N | PHE H | 505 | −5.421 | 2.194 | −53.793 | 1.00 | 58.17 | B | N |
| ATOM | 6973 | CA | PHE H | 505 | −6.683 | 1.909 | −54.464 | 1.00 | 56.56 | B | C |
| ATOM | 6974 | CB | PHE H | 505 | −7.657 | 1.198 | −53.517 | 1.00 | 20.00 | B | C |
| ATOM | 6975 | CG | PHE H | 505 | −7.174 | −0.142 | −53.045 | 1.00 | 20.00 | B | C |
| ATOM | 6976 | CD1 | PHE H | 505 | −7.297 | −1.263 | −53.851 | 1.00 | 20.00 | B | C |
| ATOM | 6977 | CE1 | PHE H | 505 | −6.817 | −2.490 | −53.432 | 1.00 | 20.00 | B | C |
| ATOM | 6978 | CZ | PHE H | 505 | −6.236 | −2.610 | −52.183 | 1.00 | 20.00 | B | C |
| ATOM | 6979 | CE2 | PHE H | 505 | −6.106 | −1.497 | −51.373 | 1.00 | 20.00 | B | C |
| ATOM | 6980 | CD2 | PHE H | 505 | −6.566 | −0.272 | −51.809 | 1.00 | 20.00 | B | C |
| ATOM | 6981 | C | PHE H | 505 | −7.307 | 3.189 | −55.020 | 1.00 | 60.12 | B | C |
| ATOM | 6982 | O | PHE H | 505 | −7.761 | 3.223 | −56.166 | 1.00 | 65.46 | B | O |
| ATOM | 6983 | N | ILE H | 506 | −7.214 | 4.267 | −54.247 | 1.00 | 56.10 | B | N |
| ATOM | 6984 | CA | ILE H | 506 | −7.762 | 5.552 | −54.667 | 1.00 | 57.32 | B | C |
| ATOM | 6985 | CB | ILE H | 506 | −7.716 | 6.599 | −53.536 | 1.00 | 20.00 | B | C |
| ATOM | 6986 | CG1 | ILE H | 506 | −8.610 | 6.163 | −52.376 | 1.00 | 20.00 | B | C |
| ATOM | 6987 | CD1 | ILE H | 506 | −8.728 | 7.191 | −51.274 | 1.00 | 20.00 | B | C |
| ATOM | 6988 | CG2 | ILE H | 506 | −8.177 | 7.956 | −54.049 | 1.00 | 20.00 | B | C |
| ATOM | 6989 | C | ILE H | 506 | −7.013 | 6.084 | −55.883 | 1.00 | 67.33 | B | C |
| ATOM | 6990 | O | ILE H | 506 | −7.625 | 6.563 | −56.839 | 1.00 | 76.99 | B | O |
| ATOM | 6991 | N | ARG H | 507 | −5.694 | 5.913 | −55.876 | 1.00 | 67.87 | B | N |
| ATOM | 6992 | CA | ARG H | 507 | −4.858 | 6.338 | −56.993 | 1.00 | 62.11 | B | C |
| ATOM | 6993 | CB | ARG H | 507 | −3.381 | 6.153 | −56.657 | 1.00 | 20.00 | B | C |
| ATOM | 6994 | CG | ARG H | 507 | −2.862 | 7.130 | −55.630 | 1.00 | 20.00 | B | C |
| ATOM | 6995 | CD | ARG H | 507 | −1.380 | 6.930 | −55.386 | 1.00 | 20.00 | B | C |
| ATOM | 6996 | NE | ARG H | 507 | −0.903 | 7.783 | −54.303 | 1.00 | 20.00 | B | N |
| ATOM | 6997 | CZ | ARG H | 507 | 0.322 | 7.729 | −53.792 | 1.00 | 20.00 | B | C |
| ATOM | 6998 | NH1 | ARG H | 507 | 1.215 | 6.883 | −54.288 | 1.00 | 20.00 | B | N |
| ATOM | 6999 | NH2 | ARG H | 507 | 0.656 | 8.530 | −52.791 | 1.00 | 20.00 | B | N |
| ATOM | 7000 | C | ARG H | 507 | −5.191 | 5.565 | −58.258 | 1.00 | 67.30 | B | C |
| ATOM | 7001 | O | ARG H | 507 | −5.255 | 6.139 | −59.344 | 1.00 | 67.02 | B | O |
| ATOM | 7002 | N | LYS H | 508 | −5.222 | 4.243 | −58.139 | 1.00 | 66.83 | B | N |
| ATOM | 7003 | CA | LYS H | 508 | −5.694 | 3.391 | −59.220 | 1.00 | 68.12 | B | C |
| ATOM | 7004 | CB | LYS H | 508 | −5.743 | 1.932 | −58.763 | 1.00 | 20.00 | B | C |
| ATOM | 7005 | CG | LYS H | 508 | −4.368 | 1.325 | −58.543 | 1.00 | 20.00 | B | C |
| ATOM | 7006 | CD | LYS H | 508 | −4.433 | −0.159 | −58.230 | 1.00 | 20.00 | B | C |
| ATOM | 7007 | CE | LYS H | 508 | −3.037 | −0.710 | −57.973 | 1.00 | 20.00 | B | C |
| ATOM | 7008 | NZ | LYS H | 508 | −3.053 | −2.129 | −57.525 | 1.00 | 20.00 | B | N |
| ATOM | 7009 | C | LYS H | 508 | −7.065 | 3.839 | −59.705 | 1.00 | 65.22 | B | C |
| ATOM | 7010 | O | LYS H | 508 | −7.245 | 4.154 | −60.881 | 1.00 | 72.34 | B | O |
| ATOM | 7011 | N | SER H | 509 | −7.996 | 3.977 | −58.767 | 1.00 | 64.87 | B | N |
| ATOM | 7012 | CA | SER H | 509 | −9.366 | 4.349 | −59.095 | 1.00 | 56.47 | B | C |
| ATOM | 7013 | CB | SER H | 509 | −10.227 | 4.420 | −57.832 | 1.00 | 20.00 | B | C |
| ATOM | 7014 | OG | SER H | 509 | −9.862 | 5.523 | −57.023 | 1.00 | 20.00 | B | O |
| ATOM | 7015 | C | SER H | 509 | −9.418 | 5.673 | −59.851 | 1.00 | 56.96 | B | C |
| ATOM | 7016 | O | SER H | 509 | −10.124 | 5.796 | −60.854 | 1.00 | 65.57 | B | O |
| ATOM | 7017 | N | ASP H | 510 | −8.670 | 6.661 | −59.368 | 1.00 | 64.34 | B | N |
| ATOM | 7018 | CA | ASP H | 510 | −8.624 | 7.971 | −60.009 | 1.00 | 64.83 | B | C |
| ATOM | 7019 | CB | ASP H | 510 | −7.774 | 8.944 | −59.192 | 1.00 | 20.00 | B | C |
| ATOM | 7020 | CG | ASP H | 510 | −8.421 | 9.315 | −57.872 | 1.00 | 20.00 | B | C |
| ATOM | 7021 | OD1 | ASP H | 510 | −9.589 | 8.927 | −57.652 | 1.00 | 20.00 | B | O |
| ATOM | 7022 | OD2 | ASP H | 510 | −7.729 | 9.904 | −57.015 | 1.00 | 20.00 | B | O |
| ATOM | 7023 | C | ASP H | 510 | −8.087 | 7.873 | −61.431 | 1.00 | 62.11 | B | C |
| ATOM | 7024 | O | ASP H | 510 | −8.711 | 8.358 | −62.373 | 1.00 | 67.61 | B | O |
| ATOM | 7025 | N | GLU H | 511 | −6.939 | 7.222 | −61.584 | 1.00 | 64.95 | B | N |
| ATOM | 7026 | CA | GLU H | 511 | −6.330 | 7.042 | −62.896 | 1.00 | 60.64 | B | C |
| ATOM | 7027 | CB | GLU H | 511 | −5.117 | 6.109 | −62.804 | 1.00 | 20.00 | B | C |
| ATOM | 7028 | CG | GLU H | 511 | −3.959 | 6.673 | −61.982 | 1.00 | 20.00 | B | C |
| ATOM | 7029 | CD | GLU H | 511 | −2.858 | 5.655 | −61.716 | 1.00 | 20.00 | B | C |
| ATOM | 7030 | OE1 | GLU H | 511 | −3.140 | 4.438 | −61.756 | 1.00 | 20.00 | B | O |
| ATOM | 7031 | OE2 | GLU H | 511 | −1.749 | 6.076 | −61.324 | 1.00 | 20.00 | B | O |
| ATOM | 7032 | C | GLU H | 511 | −7.340 | 6.528 | −63.928 | 1.00 | 59.91 | B | C |
| ATOM | 7033 | O | GLU H | 511 | −7.417 | 7.043 | −65.045 | 1.00 | 55.45 | B | O |
| ATOM | 7034 | N | LEU H | 512 | −8.137 | 5.537 | −63.533 | 1.00 | 44.35 | B | N |
| ATOM | 7035 | CA | LEU H | 512 | −9.117 | 4.929 | −64.432 | 1.00 | 49.60 | B | C |
| ATOM | 7036 | CB | LEU H | 512 | −9.744 | 3.682 | −63.796 | 1.00 | 20.00 | B | C |
| ATOM | 7037 | CG | LEU H | 512 | −8.841 | 2.462 | −63.572 | 1.00 | 20.00 | B | C |
| ATOM | 7038 | CD1 | LEU H | 512 | −9.594 | 1.368 | −62.825 | 1.00 | 20.00 | B | C |
| ATOM | 7039 | CD2 | LEU H | 512 | −8.295 | 1.930 | −64.892 | 1.00 | 20.00 | B | C |
| ATOM | 7040 | C | LEU H | 512 | −10.208 | 5.920 | −64.821 | 1.00 | 58.00 | B | C |
| ATOM | 7041 | O | LEU H | 512 | −10.524 | 6.073 | −66.000 | 1.00 | 55.11 | B | O |
| ATOM | 7042 | N | LEU H | 513 | −10.814 | 6.552 | −63.820 | 1.00 | 57.91 | B | N |
| ATOM | 7043 | CA | LEU H | 513 | −11.866 | 7.536 | −64.057 | 1.00 | 57.49 | B | C |
| ATOM | 7044 | CB | LEU H | 513 | −12.390 | 8.097 | −62.733 | 1.00 | 20.00 | B | C |
| ATOM | 7045 | CG | LEU H | 513 | −13.077 | 7.088 | −61.815 | 1.00 | 20.00 | B | C |
| ATOM | 7046 | CD1 | LEU H | 513 | −13.388 | 7.705 | −60.461 | 1.00 | 20.00 | B | C |
| ATOM | 7047 | CD2 | LEU H | 513 | −14.336 | 6.556 | −62.474 | 1.00 | 20.00 | B | C |
| ATOM | 7048 | C | LEU H | 513 | −11.384 | 8.676 | −64.949 | 1.00 | 58.78 | B | C |
| ATOM | 7049 | O | LEU H | 513 | −12.154 | 9.220 | −65.740 | 1.00 | 59.88 | B | O |
| ATOM | 7050 | N | HIS H | 514 | −10.131 | 9.086 | −64.764 | 1.00 | 60.07 | B | N |
| ATOM | 7051 | CA | HIS H | 514 | −9.544 | 10.151 | −65.578 | 1.00 | 62.82 | B | C |
| ATOM | 7052 | CB | HIS H | 514 | −8.240 | 10.665 | −64.951 | 1.00 | 20.00 | B | C |
| ATOM | 7053 | CG | HIS H | 514 | −8.406 | 11.218 | −63.567 | 1.00 | 20.00 | B | C |
| ATOM | 7054 | ND1 | HIS H | 514 | −9.083 | 12.390 | −63.308 | 1.00 | 20.00 | B | N |
| ATOM | 7055 | CE1 | HIS H | 514 | −9.060 | 12.634 | −62.010 | 1.00 | 20.00 | B | C |
| ATOM | 7056 | NE2 | HIS H | 514 | −8.393 | 11.660 | −61.416 | 1.00 | 20.00 | B | N |
| ATOM | 7057 | CD2 | HIS H | 514 | −7.960 | 10.770 | −62.368 | 1.00 | 20.00 | B | C |
| ATOM | 7058 | C | HIS H | 514 | −9.305 | 9.679 | −67.014 | 1.00 | 65.10 | B | C |
| ATOM | 7059 | O | HIS H | 514 | −9.352 | 10.475 | −67.953 | 1.00 | 66.73 | B | O |
| ATOM | 7060 | N | ASN H | 515 | −9.179 | 8.365 | −67.187 | 1.00 | 63.45 | B | N |
| ATOM | 7061 | CA | ASN H | 515 | −9.028 | 7.768 | −68.513 | 1.00 | 56.53 | B | C |
| ATOM | 7062 | CB | ASN H | 515 | −8.367 | 6.392 | −68.409 | 1.00 | 20.00 | B | C |
| ATOM | 7063 | CG | ASN H | 515 | −6.901 | 6.476 | −68.035 | 1.00 | 20.00 | B | C |
| ATOM | 7064 | OD1 | ASN H | 515 | −6.233 | 7.470 | −68.319 | 1.00 | 20.00 | B | O |
| ATOM | 7065 | ND2 | ASN H | 515 | −6.375 | 5.399 | −67.462 | 1.00 | 20.00 | B | N |
| ATOM | 7066 | C | ASN H | 515 | −10.357 | 7.651 | −69.254 | 1.00 | 60.06 | B | C |
| ATOM | 7067 | O | ASN H | 515 | −10.414 | 7.816 | −70.473 | 1.00 | 66.48 | B | O |
| ATOM | 7068 | N | VAL H | 516 | −11.402 | 7.275 | −68.522 | 1.00 | 54.29 | B | N |
| ATOM | 7069 | CA | VAL H | 516 | −12.765 | 7.256 | −69.056 | 1.00 | 52.70 | B | C |
| ATOM | 7070 | CB | VAL H | 516 | −13.775 | 6.751 | −68.001 | 1.00 | 20.00 | B | C |
| ATOM | 7071 | CG1 | VAL H | 516 | −15.194 | 6.825 | −68.541 | 1.00 | 20.00 | B | C |
| ATOM | 7072 | CG2 | VAL H | 516 | −13.431 | 5.333 | −67.568 | 1.00 | 20.00 | B | C |
| ATOM | 7073 | C | VAL H | 516 | −13.206 | 8.633 | −69.557 | 1.00 | 60.10 | B | C |
| ATOM | 7074 | O | VAL H | 516 | −13.761 | 8.759 | −70.649 | 1.00 | 61.55 | B | O |
| ATOM | 7075 | N | ASN H | 517 | −12.957 | 9.661 | −68.752 | 1.00 | 69.10 | B | N |
| ATOM | 7076 | CA | ASN H | 517 | −13.297 | 11.030 | −69.125 | 1.00 | 67.75 | B | C |
| ATOM | 7077 | CB | ASN H | 517 | −12.850 | 12.012 | −68.038 | 1.00 | 20.00 | B | C |
| ATOM | 7078 | CG | ASN H | 517 | −13.685 | 11.907 | −66.774 | 1.00 | 20.00 | B | C |
| ATOM | 7079 | OD1 | ASN H | 517 | −14.835 | 11.471 | −66.813 | 1.00 | 20.00 | B | O |
| ATOM | 7080 | ND2 | ASN H | 517 | −13.126 | 12.355 | −65.653 | 1.00 | 20.00 | B | N |
| ATOM | 7081 | C | ASN H | 517 | −12.686 | 11.424 | −70.465 | 1.00 | 71.17 | B | C |
| ATOM | 7082 | O | ASN H | 517 | −13.289 | 12.169 | −71.238 | 1.00 | 66.42 | B | O |
| ATOM | 7083 | N | ALA H | 518 | −11.489 | 10.912 | −70.739 | 1.00 | 72.11 | B | N |
| ATOM | 7084 | CA | ALA H | 518 | −10.794 | 11.202 | −71.989 | 1.00 | 74.82 | B | C |
| ATOM | 7085 | CB | ALA H | 518 | −9.318 | 10.855 | −71.867 | 1.00 | 20.00 | B | C |
| ATOM | 7086 | C | ALA H | 518 | −11.421 | 10.450 | −73.155 | 1.00 | 70.11 | B | C |
| ATOM | 7087 | O | ALA H | 518 | −11.593 | 11.001 | −74.242 | 1.00 | 65.18 | B | O |
| ATOM | 7088 | N | GLY H | 519 | −11.665 | 9.160 | −72.952 | 1.00 | 56.07 | B | N |
| ATOM | 7089 | CA | GLY H | 519 | −12.395 | 8.360 | −73.926 | 1.00 | 53.01 | B | C |
| ATOM | 7090 | C | GLY H | 519 | −13.763 | 8.941 | −74.222 | 1.00 | 63.58 | B | C |
| ATOM | 7091 | O | GLY H | 519 | −14.203 | 8.960 | −75.371 | 1.00 | 69.35 | B | O |
| ATOM | 7092 | N | LYS H | 520 | −14.422 | 9.444 | −73.183 | 1.00 | 58.33 | B | N |
| ATOM | 7093 | CA | LYS H | 520 | −15.716 | 10.101 | −73.333 | 1.00 | 53.44 | B | C |
| ATOM | 7094 | CB | LYS H | 520 | −16.241 | 10.557 | −71.975 | 1.00 | 20.00 | B | C |
| ATOM | 7095 | CG | LYS H | 520 | −16.441 | 9.429 | −70.996 | 1.00 | 20.00 | B | C |
| ATOM | 7096 | CD | LYS H | 520 | −16.739 | 9.961 | −69.616 | 1.00 | 20.00 | B | C |
| ATOM | 7097 | CE | LYS H | 520 | −17.296 | 8.868 | −68.730 | 1.00 | 20.00 | B | C |
| ATOM | 7098 | NZ | LYS H | 520 | −17.807 | 9.424 | −67.452 | 1.00 | 20.00 | B | N |
| ATOM | 7099 | C | LYS H | 520 | −15.623 | 11.296 | −74.268 | 1.00 | 58.24 | B | C |
| ATOM | 7100 | O | LYS H | 520 | −16.579 | 11.621 | −74.972 | 1.00 | 48.60 | B | O |
| ATOM | 7101 | N | SER H | 521 | −14.489 | 11.986 | −74.219 | 1.00 | 67.19 | B | N |
| ATOM | 7102 | CA | SER H | 521 | −14.232 | 13.101 | −75.120 | 1.00 | 60.42 | B | C |
| ATOM | 7103 | CB | SER H | 521 | −12.938 | 13.821 | −74.727 | 1.00 | 20.00 | B | C |
| ATOM | 7104 | OG | SER H | 521 | −11.894 | 12.898 | −74.459 | 1.00 | 20.00 | B | O |
| ATOM | 7105 | C | SER H | 521 | −14.167 | 12.636 | −76.573 | 1.00 | 54.19 | B | C |
| ATOM | 7106 | O | SER H | 521 | −14.886 | 13.151 | −77.430 | 1.00 | 65.36 | B | O |
| ATOM | 7107 | N | THR H | 522 | −13.362 | 11.609 | −76.825 | 1.00 | 50.09 | B | N |
| ATOM | 7108 | CA | THR H | 522 | −13.259 | 11.025 | −78.158 | 1.00 | 57.59 | B | C |
| ATOM | 7109 | CB | THR H | 522 | −12.299 | 9.817 | −78.179 | 1.00 | 20.00 | B | C |
| ATOM | 7110 | OG1 | THR H | 522 | −12.774 | 8.810 | −77.278 | 1.00 | 20.00 | B | O |
| ATOM | 7111 | CG2 | THR H | 522 | −10.894 | 10.235 | −77.767 | 1.00 | 20.00 | B | C |
| ATOM | 7112 | C | THR H | 522 | −14.625 | 10.565 | −78.646 | 1.00 | 59.16 | B | C |
| ATOM | 7113 | O | THR H | 522 | −15.044 | 10.893 | −79.755 | 1.00 | 65.90 | B | O |
| ATOM | 7114 | N | THR H | 523 | −15.338 | 9.849 | −77.783 | 1.00 | 52.59 | B | N |
| ATOM | 7115 | CA | THR H | 523 | −16.665 | 9.347 | −78.112 | 1.00 | 42.19 | B | C |
| ATOM | 7116 | CB | THR H | 523 | −17.298 | 8.599 | −76.922 | 1.00 | 20.00 | B | C |
| ATOM | 7117 | OG1 | THR H | 523 | −17.543 | 9.516 | −75.848 | 1.00 | 20.00 | B | O |
| ATOM | 7118 | CG2 | THR H | 523 | −16.373 | 7.492 | −76.435 | 1.00 | 20.00 | B | C |
| ATOM | 7119 | C | THR H | 523 | −17.596 | 10.466 | −78.566 | 1.00 | 48.90 | B | C |
| ATOM | 7120 | O | THR H | 523 | −18.331 | 10.314 | −79.541 | 1.00 | 63.19 | B | O |
| ATOM | 7121 | N | ASN H | 524 | −17.535 | 11.601 | −77.881 | 1.00 | 52.51 | B | N |
| ATOM | 7122 | CA | ASN H | 524 | −18.341 | 12.752 | −78.258 | 1.00 | 57.54 | B | C |
| ATOM | 7123 | CB | ASN H | 524 | −18.235 | 13.853 | −77.202 | 1.00 | 20.00 | B | C |
| ATOM | 7124 | CG | ASN H | 524 | −19.015 | 13.528 | −75.945 | 1.00 | 20.00 | B | C |
| ATOM | 7125 | OD1 | ASN H | 524 | −19.910 | 12.682 | −75.959 | 1.00 | 20.00 | B | O |
| ATOM | 7126 | ND2 | ASN H | 524 | −18.683 | 14.203 | −74.850 | 1.00 | 20.00 | B | N |
| ATOM | 7127 | C | ASN H | 524 | −17.956 | 13.287 | −79.633 | 1.00 | 54.12 | B | C |
| ATOM | 7128 | O | ASN H | 524 | −18.812 | 13.463 | −80.500 | 1.00 | 61.31 | B | O |
| ATOM | 7129 | N | SER H | 525 | −16.656 | 13.422 | −79.866 | 1.00 | 48.97 | B | N |
| ATOM | 7130 | CA | SER H | 525 | −16.165 | 13.913 | −81.145 | 1.00 | 64.81 | B | C |
| ATOM | 7131 | CB | SER H | 525 | −14.636 | 13.940 | −81.151 | 1.00 | 61.22 | B | C |
| ATOM | 7132 | OG | SER H | 525 | −14.141 | 14.856 | −80.191 | 1.00 | 85.90 | B | O |
| ATOM | 7133 | C | SER H | 525 | −16.689 | 13.053 | −82.292 | 1.00 | 69.67 | B | C |
| ATOM | 7134 | O | SER H | 525 | −17.013 | 13.563 | −83.367 | 1.00 | 72.60 | B | O |
| ATOM | 7135 | N | LYS H | 526 | −16.849 | 11.760 | −82.029 | 1.00 | 66.06 | B | N |
| ATOM | 7136 | CA | LYS H | 526 | −17.335 | 10.831 | −83.043 | 1.00 | 63.67 | B | C |
| ATOM | 7137 | CB | LYS H | 526 | −17.003 | 9.383 | −82.660 | 1.00 | 70.55 | B | C |
| ATOM | 7138 | CG | LYS H | 526 | −15.562 | 8.963 | −82.939 | 1.00 | 75.29 | B | C |
| ATOM | 7139 | CD | LYS H | 526 | −15.205 | 7.681 | −82.194 | 1.00 | 85.03 | B | C |
| ATOM | 7140 | CE | LYS H | 526 | −13.700 | 7.525 | −82.041 | 1.00 | 82.65 | B | C |
| ATOM | 7141 | NZ | LYS H | 526 | −13.347 | 6.537 | −80.984 | 1.00 | 73.89 | B | N |
| ATOM | 7142 | C | LYS H | 526 | −18.838 | 10.987 | −83.239 | 1.00 | 55.36 | B | C |
| ATOM | 7143 | O | LYS H | 526 | −19.333 | 10.950 | −84.365 | 1.00 | 70.69 | B | O |
| ATOM | 7144 | N | ILE H | 527 | −19.560 | 11.149 | −82.135 | 1.00 | 57.52 | B | N |
| ATOM | 7145 | CA | ILE H | 527 | −20.995 | 11.389 | −82.189 | 1.00 | 58.48 | B | C |
| ATOM | 7146 | CB | ILE H | 527 | −21.569 | 11.642 | −80.787 | 1.00 | 57.00 | B | C |
| ATOM | 7147 | CG1 | ILE H | 527 | −21.356 | 10.420 | −79.891 | 1.00 | 49.07 | B | C |
| ATOM | 7148 | CD1 | ILE H | 527 | −21.790 | 9.118 | −80.517 | 1.00 | 46.18 | B | C |
| ATOM | 7149 | CG2 | ILE H | 527 | −23.035 | 12.029 | −80.872 | 1.00 | 52.63 | B | C |
| ATOM | 7150 | C | ILE H | 527 | −21.317 | 12.586 | −83.075 | 1.00 | 65.20 | B | C |
| ATOM | 7151 | O | ILE H | 527 | −22.228 | 12.532 | −83.900 | 1.00 | 66.48 | B | O |
| ATOM | 7152 | N | TYR H | 528 | −20.590 | 13.681 | −82.870 | 1.00 | 69.78 | B | N |
| ATOM | 7153 | CA | TYR H | 528 | −20.780 | 14.884 | −83.673 | 1.00 | 72.52 | B | C |
| ATOM | 7154 | CB | TYR H | 528 | −19.738 | 15.948 | −83.316 | 1.00 | 73.16 | B | C |
| ATOM | 7155 | CG | TYR H | 528 | −19.707 | 16.343 | −81.856 | 1.00 | 76.94 | B | C |
| ATOM | 7156 | CD1 | TYR H | 528 | −18.615 | 17.018 | −81.327 | 1.00 | 70.95 | B | C |
| ATOM | 7157 | CE1 | TYR H | 528 | −18.584 | 17.406 | −80.000 | 1.00 | 67.73 | B | C |
| ATOM | 7158 | CZ | TYR H | 528 | −19.653 | 17.114 | −79.180 | 1.00 | 71.89 | B | C |
| ATOM | 7159 | OH | TYR H | 528 | −19.626 | 17.490 | −77.856 | 1.00 | 75.44 | B | O |
| ATOM | 7160 | CE2 | TYR H | 528 | −20.759 | 16.464 | −79.687 | 1.00 | 77.03 | B | C |
| ATOM | 7161 | CD2 | TYR H | 528 | −20.782 | 16.084 | −81.018 | 1.00 | 77.84 | B | C |
| ATOM | 7162 | C | TYR H | 528 | −20.678 | 14.551 | −85.157 | 1.00 | 71.63 | B | C |
| ATOM | 7163 | O | TYR H | 528 | −21.609 | 14.797 | −85.925 | 1.00 | 84.20 | B | O |
| ATOM | 7164 | N | HIS H | 529 | −19.526 | 14.025 | −85.559 | 1.00 | 67.69 | B | N |
| ATOM | 7165 | CA | HIS H | 529 | −19.265 | 13.733 | −86.962 | 1.00 | 60.28 | B | C |
| ATOM | 7166 | CB | HIS H | 529 | −17.883 | 13.102 | −87.131 | 1.00 | 67.84 | B | C |
| ATOM | 7167 | CG | HIS H | 529 | −16.752 | 14.042 | −86.853 | 1.00 | 88.54 | B | C |
| ATOM | 7168 | ND1 | HIS H | 529 | −15.514 | 13.914 | −87.444 | 1.00 | 93.34 | B | N |
| ATOM | 7169 | CE1 | HIS H | 529 | −14.718 | 14.874 | −87.007 | 1.00 | 86.23 | B | C |
| ATOM | 7170 | NE2 | HIS H | 529 | −15.394 | 15.614 | −86.145 | 1.00 | 91.95 | B | N |
| ATOM | 7171 | CD2 | HIS H | 529 | −16.667 | 15.110 | −86.026 | 1.00 | 93.77 | B | C |
| ATOM | 7172 | C | HIS H | 529 | −20.333 | 12.817 | −87.546 | 1.00 | 63.30 | B | C |
| ATOM | 7173 | O | HIS H | 529 | −20.803 | 13.034 | −88.663 | 1.00 | 61.02 | B | O |
| ATOM | 7174 | N | ILE H | 530 | −20.710 | 11.791 | −86.791 | 1.00 | 59.79 | B | N |
| ATOM | 7175 | CA | ILE H | 530 | −21.800 | 10.915 | −87.201 | 1.00 | 45.70 | B | C |
| ATOM | 7176 | CB | ILE H | 530 | −22.019 | 9.780 | −86.190 | 1.00 | 40.57 | B | C |
| ATOM | 7177 | CG1 | ILE H | 530 | −20.879 | 8.765 | −86.292 | 1.00 | 42.86 | B | C |
| ATOM | 7178 | CD1 | ILE H | 530 | −20.826 | 7.775 | −85.154 | 1.00 | 51.01 | B | C |
| ATOM | 7179 | CG2 | ILE H | 530 | −23.360 | 9.108 | −86.437 | 1.00 | 35.31 | B | C |
| ATOM | 7180 | C | ILE H | 530 | −23.085 | 11.717 | −87.331 | 1.00 | 57.52 | B | C |
| ATOM | 7181 | O | ILE H | 530 | −23.779 | 11.645 | −88.345 | 1.00 | 71.18 | B | O |
| ATOM | 7182 | N | GLU H | 531 | −23.322 | 12.569 | −86.341 | 1.00 | 67.55 | B | N |
| ATOM | 7183 | CA | GLU H | 531 | −24.479 | 13.449 | −86.325 | 1.00 | 69.79 | B | C |
| ATOM | 7184 | CB | GLU H | 531 | −24.440 | 14.327 | −85.074 | 1.00 | 84.02 | B | C |
| ATOM | 7185 | CG | GLU H | 531 | −25.803 | 14.718 | −84.541 | 1.00 | 104.22 | B | C |
| ATOM | 7186 | CD | GLU H | 531 | −25.706 | 15.531 | −83.270 | 1.00 | 116.08 | B | C |
| ATOM | 7187 | OE1 | GLU H | 531 | −25.017 | 15.079 | −82.331 | 1.00 | 118.71 | B | O |
| ATOM | 7188 | OE2 | GLU H | 531 | −26.222 | 16.669 | −83.251 | 1.00 | 119.69 | B | O |
| ATOM | 7189 | C | GLU H | 531 | −24.506 | 14.328 | −87.569 | 1.00 | 64.87 | B | C |
| ATOM | 7190 | O | GLU H | 531 | −25.517 | 14.404 | −88.269 | 1.00 | 55.58 | B | O |
| ATOM | 7191 | N | ASN H | 532 | −23.394 | 15.011 | −87.821 | 1.00 | 65.70 | B | N |
| ATOM | 7192 | CA | ASN H | 532 | −23.273 | 15.890 | −88.979 | 1.00 | 64.16 | B | C |
| ATOM | 7193 | CB | ASN H | 532 | −21.931 | 16.625 | −88.953 | 1.00 | 60.58 | B | C |
| ATOM | 7194 | CG | ASN H | 532 | −21.793 | 17.621 | −90.085 | 1.00 | 67.75 | B | C |
| ATOM | 7195 | OD1 | ASN H | 532 | −22.591 | 18.550 | −90.206 | 1.00 | 53.59 | B | O |
| ATOM | 7196 | ND2 | ASN H | 532 | −20.822 | 17.392 | −90.961 | 1.00 | 61.81 | B | N |
| ATOM | 7197 | C | ASN H | 532 | −23.418 | 15.119 | −90.284 | 1.00 | 72.17 | B | C |
| ATOM | 7198 | O | ASN H | 532 | −24.041 | 15.593 | −91.233 | 1.00 | 85.75 | B | O |
| ATOM | 7199 | N | GLU H | 533 | −22.870 | 13.909 | −90.300 | 1.00 | 79.68 | B | N |
| ATOM | 7200 | CA | GLU H | 533 | −22.922 | 13.044 | −91.468 | 1.00 | 71.96 | B | C |
| ATOM | 7201 | CB | GLU H | 533 | −22.010 | 11.837 | −91.264 | 1.00 | 67.87 | B | C |
| ATOM | 7202 | CG | GLU H | 533 | −21.638 | 11.131 | −92.547 | 1.00 | 81.94 | B | C |
| ATOM | 7203 | CD | GLU H | 533 | −20.906 | 12.037 | −93.515 | 1.00 | 92.79 | B | C |
| ATOM | 7204 | OE1 | GLU H | 533 | −19.930 | 12.692 | −93.094 | 1.00 | 95.90 | B | O |
| ATOM | 7205 | OE2 | GLU H | 533 | −21.247 | 12.025 | −94.716 | 1.00 | 93.14 | B | O |
| ATOM | 7206 | C | GLU H | 533 | −24.342 | 12.570 | −91.747 | 1.00 | 68.54 | B | C |
| ATOM | 7207 | O | GLU H | 533 | −24.667 | 12.175 | −92.866 | 1.00 | 79.29 | B | O |
| ATOM | 7208 | N | ILE H | 534 | −25.137 | 12.470 | −90.688 | 1.00 | 69.50 | B | N |
| ATOM | 7209 | CA | ILE H | 534 | −26.529 | 12.076 | −90.817 | 1.00 | 72.55 | B | C |
| ATOM | 7210 | CB | ILE H | 534 | −27.056 | 11.467 | −89.514 | 1.00 | 79.55 | B | C |
| ATOM | 7211 | CG1 | ILE H | 534 | −26.263 | 10.209 | −89.164 | 1.00 | 80.41 | B | C |
| ATOM | 7212 | CD1 | ILE H | 534 | −25.562 | 9.592 | −90.348 | 1.00 | 89.38 | B | C |
| ATOM | 7213 | CG2 | ILE H | 534 | −28.526 | 11.138 | −89.645 | 1.00 | 78.63 | B | C |
| ATOM | 7214 | C | ILE H | 534 | −27.389 | 13.273 | −91.189 | 1.00 | 74.92 | B | C |
| ATOM | 7215 | O | ILE H | 534 | −28.479 | 13.120 | −91.740 | 1.00 | 81.01 | B | O |
| ATOM | 7216 | N | ALA H | 535 | −26.880 | 14.465 | −90.903 | 1.00 | 79.52 | B | N |
| ATOM | 7217 | CA | ALA H | 535 | −27.559 | 15.690 | −91.290 | 1.00 | 82.64 | B | C |
| ATOM | 7218 | CB | ALA H | 535 | −27.016 | 16.867 | −90.498 | 1.00 | 75.39 | B | C |
| ATOM | 7219 | C | ALA H | 535 | −27.440 | 15.939 | −92.795 | 1.00 | 81.39 | B | C |
| ATOM | 7220 | O | ALA H | 535 | −28.408 | 16.341 | −93.443 | 1.00 | 85.54 | B | O |
| ATOM | 7221 | N | ARG H | 536 | −26.274 | 15.630 | −93.358 | 1.00 | 78.41 | B | N |
| ATOM | 7222 | CA | ARG H | 536 | −26.044 | 15.812 | −94.791 | 1.00 | 83.27 | B | C |
| ATOM | 7223 | CB | ARG H | 536 | −24.545 | 15.814 | −95.106 | 1.00 | 84.58 | B | C |
| ATOM | 7224 | CG | ARG H | 536 | −23.776 | 16.993 | −94.527 | 1.00 | 103.24 | B | C |
| ATOM | 7225 | CD | ARG H | 536 | −22.638 | 17.422 | −95.449 | 1.00 | 114.72 | B | C |
| ATOM | 7226 | NE | ARG H | 536 | −21.692 | 16.343 | −95.722 | 1.00 | 117.80 | B | N |
| ATOM | 7227 | CZ | ARG H | 536 | −20.692 | 16.430 | −96.594 | 1.00 | 116.66 | B | C |
| ATOM | 7228 | NH1 | ARG H | 536 | −20.522 | 17.538 | −97.303 | 1.00 | 114.57 | B | N |
| ATOM | 7229 | NH2 | ARG H | 536 | −19.885 | 15.396 | −96.786 | 1.00 | 116.38 | B | N |
| ATOM | 7230 | C | ARG H | 536 | −26.744 | 14.727 | −95.606 | 1.00 | 82.20 | B | C |
| ATOM | 7231 | O | ARG H | 536 | −27.369 | 15.011 | −96.627 | 1.00 | 86.28 | B | O |
| ATOM | 7232 | N | ILE H | 537 | −26.598 | 13.479 | −95.172 | 1.00 | 77.08 | B | N |
| ATOM | 7233 | CA | ILE H | 537 | −27.315 | 12.371 | −95.782 | 1.00 | 66.79 | B | C |
| ATOM | 7234 | CB | ILE H | 537 | −26.955 | 11.038 | −95.113 | 1.00 | 64.76 | B | C |
| ATOM | 7235 | CG1 | ILE H | 537 | −25.523 | 10.627 | −95.476 | 1.00 | 66.92 | B | C |
| ATOM | 7236 | CD1 | ILE H | 537 | −24.906 | 9.625 | −94.519 | 1.00 | 70.83 | B | C |
| ATOM | 7237 | CG2 | ILE H | 537 | −27.960 | 9.964 | −95.497 | 1.00 | 51.02 | B | C |
| ATOM | 7238 | C | ILE H | 537 | −28.817 | 12.585 | −95.674 | 1.00 | 74.94 | B | C |
| ATOM | 7239 | O | ILE H | 537 | −29.587 | 12.070 | −96.482 | 1.00 | 97.16 | B | O |
| ATOM | 7240 | N | LYS H | 538 | −29.232 | 13.337 | −94.661 | 1.00 | 75.09 | B | N |
| ATOM | 7241 | CA | LYS H | 538 | −30.640 | 13.666 | −94.485 | 1.00 | 90.28 | B | C |
| ATOM | 7242 | CB | LYS H | 538 | −30.958 | 13.868 | −93.003 | 1.00 | 93.73 | B | C |
| ATOM | 7243 | CG | LYS H | 538 | −32.344 | 13.390 | −92.575 | 1.00 | 96.60 | B | C |
| ATOM | 7244 | CD | LYS H | 538 | −32.640 | 13.787 | −91.126 | 1.00 | 95.37 | B | C |
| ATOM | 7245 | CE | LYS H | 538 | −34.127 | 13.714 | −90.798 | 1.00 | 100.63 | B | C |
| ATOM | 7246 | NZ | LYS H | 538 | −34.398 | 14.067 | −89.375 | 1.00 | 99.58 | B | N |
| ATOM | 7247 | C | LYS H | 538 | −31.022 | 14.911 | −95.284 | 1.00 | 102.20 | B | C |
| ATOM | 7248 | O | LYS H | 538 | −32.166 | 15.360 | −95.235 | 1.00 | 112.11 | B | O |
| ATOM | 7249 | N | LYS H | 539 | −30.069 | 15.444 | −96.043 | 1.00 | 105.15 | B | N |
| ATOM | 7250 | CA | LYS H | 539 | −30.317 | 16.603 | −96.899 | 1.00 | 105.65 | B | C |
| ATOM | 7251 | CB | LYS H | 539 | −29.144 | 17.589 | −96.823 | 1.00 | 110.12 | B | C |
| ATOM | 7252 | CG | LYS H | 539 | −29.408 | 18.944 | −97.481 | 1.00 | 113.31 | B | C |
| ATOM | 7253 | CD | LYS H | 539 | −28.182 | 19.854 | −97.435 | 1.00 | 114.26 | B | C |
| ATOM | 7254 | CE | LYS H | 539 | −28.426 | 21.145 | −98.205 | 1.00 | 118.39 | B | C |
| ATOM | 7255 | NZ | LYS H | 539 | −27.168 | 21.897 | −98.470 | 1.00 | 121.77 | B | N |
| ATOM | 7256 | C | LYS H | 539 | −30.572 | 16.187 | −98.347 | 1.00 | 108.88 | B | C |
| ATOM | 7257 | O | LYS H | 539 | −30.886 | 17.021 | −99.195 | 1.00 | 110.77 | B | O |
| ATOM | 7258 | N | LEU H | 540 | −30.515 | 14.884 | −98.605 | 1.00 | 111.50 | B | N |
| ATOM | 7259 | CA | LEU H | 540 | −30.796 | 14.356 | −99.935 | 1.00 | 110.31 | B | C |
| ATOM | 7260 | CB | LEU H | 540 | −29.690 | 13.389 | −100.368 | 1.00 | 101.19 | B | C |
| ATOM | 7261 | CG | LEU H | 540 | −28.270 | 13.889 | −100.078 | 1.00 | 94.26 | B | C |
| ATOM | 7262 | CD1 | LEU H | 540 | −27.249 | 12.769 | −100.179 | 1.00 | 86.40 | B | C |
| ATOM | 7263 | CD2 | LEU H | 540 | −27.896 | 15.059 | −100.984 | 1.00 | 84.54 | B | C |
| ATOM | 7264 | C | LEU H | 540 | −32.157 | 13.675 | −99.961 | 1.00 | 118.37 | B | C |
| ATOM | 7265 | O | LEU H | 540 | −32.531 | 13.039 | −100.945 | 1.00 | 116.71 | B | O |
| ATOM | 7266 | N | ILE H | 541 | −32.923 | 13.883 | −98.896 | 1.00 | 130.80 | B | N |
| ATOM | 7267 | CA | ILE H | 541 | −34.267 | 13.339 | −98.801 | 1.00 | 140.48 | B | C |
| ATOM | 7268 | CB | ILE H | 541 | −34.250 | 11.862 | −98.372 | 1.00 | 138.99 | B | C |
| ATOM | 7269 | CG1 | ILE H | 541 | −33.319 | 11.059 | −99.282 | 1.00 | 133.30 | B | C |
| ATOM | 7270 | CD1 | ILE H | 541 | −33.895 | 10.789 | −100.656 | 1.00 | 136.26 | B | C |
| ATOM | 7271 | CG2 | ILE H | 541 | −35.653 | 11.281 | −98.432 | 1.00 | 136.12 | B | C |
| ATOM | 7272 | C | ILE H | 541 | −35.146 | 14.139 | −97.839 | 1.00 | 144.97 | B | C |
| ATOM | 7273 | O | ILE H | 541 | −36.372 | 14.063 | −97.905 | 1.00 | 145.83 | B | O |
| ATOM | 7274 | N | GLY H | 542 | −34.523 | 14.940 | −96.979 | 1.00 | 148.77 | B | N |
| ATOM | 7275 | CA | GLY H | 542 | −35.267 | 15.725 | −95.991 | 1.00 | 153.58 | B | C |
| ATOM | 7276 | C | GLY H | 542 | −35.358 | 17.210 | −96.308 | 1.00 | 160.45 | B | C |
| ATOM | 7277 | O | GLY H | 542 | −35.594 | 17.599 | −97.454 | 1.00 | 163.41 | B | O |
| ATOM | 7278 | N | GLU H | 543 | −35.275 | 18.034 | −95.267 | 1.00 | 163.83 | B | N |
| ATOM | 7279 | CA | GLU H | 543 | −35.152 | 19.480 | −95.430 | 1.00 | 165.52 | B | C |
| ATOM | 7280 | CB | GLU H | 543 | −35.727 | 20.205 | −94.211 | 1.00 | 158.94 | B | C |
| ATOM | 7285 | C | GLU H | 543 | −33.698 | 19.890 | −95.644 | 1.00 | 168.27 | B | C |
| ATOM | 7286 | O | GLU H | 543 | −32.778 | 19.116 | −95.373 | 1.00 | 167.37 | B | O |
| ATOM | 7287 | N | GLN I | 26 | 21.181 | −26.937 | −9.836 | 1.00 | 23.62 | C | N |
| ATOM | 7288 | CA | GLN I | 26 | 21.159 | −25.618 | −10.530 | 1.00 | 23.82 | C | C |
| ATOM | 7289 | CB | GLN I | 26 | 21.607 | −24.503 | −9.580 | 1.00 | 23.94 | C | C |
| ATOM | 7290 | CG | GLN I | 26 | 20.888 | −24.492 | −8.237 | 1.00 | 28.83 | C | C |
| ATOM | 7291 | CD | GLN I | 26 | 21.323 | −25.626 | −7.329 | 1.00 | 36.36 | C | C |
| ATOM | 7292 | OE1 | GLN I | 26 | 20.813 | −26.740 | −7.428 | 1.00 | 39.09 | C | O |
| ATOM | 7293 | NE2 | GLN I | 26 | 22.207 | −25.325 | −6.384 | 1.00 | 37.86 | C | N |
| ATOM | 7294 | C | GLN I | 26 | 22.043 | −25.641 | −11.774 | 1.00 | 23.09 | C | C |
| ATOM | 7295 | O | GLN I | 26 | 22.946 | −26.471 | −11.888 | 1.00 | 24.21 | C | O |
| ATOM | 7296 | N | ASN I | 27 | 21.670 | −24.835 | −12.765 | 1.00 | 20.98 | C | N |
| ATOM | 7297 | CA | ASN I | 27 | 22.435 | −24.709 | −14.004 | 1.00 | 19.70 | C | C |
| ATOM | 7298 | CB | ASN I | 27 | 22.165 | −25.904 | −14.928 | 1.00 | 20.41 | C | C |
| ATOM | 7299 | CG | ASN I | 27 | 22.952 | −25.833 | −16.231 | 1.00 | 23.56 | C | C |
| ATOM | 7300 | OD1 | ASN I | 27 | 24.156 | −25.579 | −16.199 | 1.00 | 24.42 | C | O |
| ATOM | 7301 | ND2 | ASN I | 27 | 22.227 | −25.693 | −17.339 | 1.00 | 27.70 | C | N |
| ATOM | 7302 | C | ASN I | 27 | 22.086 | −23.414 | −14.727 | 1.00 | 18.42 | C | C |
| ATOM | 7303 | O | ASN I | 27 | 21.539 | −23.447 | −15.830 | 1.00 | 18.47 | C | O |
| ATOM | 7304 | N | ILE I | 28 | 22.230 | −22.288 | −14.033 | 1.00 | 16.54 | C | N |
| ATOM | 7305 | CA | ILE I | 28 | 21.853 | −21.004 | −14.611 | 1.00 | 14.59 | C | C |
| ATOM | 7306 | CB | ILE I | 28 | 21.526 | −19.932 | −13.546 | 1.00 | 14.01 | C | C |
| ATOM | 7307 | CG1 | ILE I | 28 | 22.461 | −20.055 | −12.343 | 1.00 | 15.60 | C | C |
| ATOM | 7308 | CD1 | ILE I | 28 | 23.710 | −19.205 | −12.454 | 1.00 | 16.26 | C | C |
| ATOM | 7309 | CG2 | ILE I | 28 | 20.065 | −20.023 | −13.122 | 1.00 | 12.57 | C | C |
| ATOM | 7310 | C | ILE I | 28 | 22.915 | −20.484 | −15.561 | 1.00 | 13.86 | C | C |
| ATOM | 7311 | O | ILE I | 28 | 24.111 | −20.702 | −15.363 | 1.00 | 13.70 | C | O |
| ATOM | 7312 | N | THR I | 29 | 22.442 | −20.013 | −16.703 | 1.00 | 13.23 | C | N |
| ATOM | 7313 | CA | THR I | 29 | 23.301 | −19.589 | −17.786 | 1.00 | 12.65 | C | C |
| ATOM | 7314 | CB | THR I | 29 | 23.241 | −20.586 | −18.948 | 1.00 | 12.69 | C | C |
| ATOM | 7315 | OG1 | THR I | 29 | 22.805 | −21.861 | −18.463 | 1.00 | 12.62 | C | O |
| ATOM | 7316 | CG2 | THR I | 29 | 24.603 | −20.727 | −19.608 | 1.00 | 12.92 | C | C |
| ATOM | 7317 | C | THR I | 29 | 22.740 | −18.277 | −18.281 | 1.00 | 12.47 | C | C |
| ATOM | 7318 | O | THR I | 29 | 21.531 | −18.051 | −18.220 | 1.00 | 12.53 | C | O |
| ATOM | 7319 | N | GLU I | 30 | 23.596 | −17.467 | −18.884 | 1.00 | 11.98 | C | N |
| ATOM | 7320 | CA | GLU I | 30 | 23.135 | −16.364 | −19.702 | 1.00 | 11.68 | C | C |
| ATOM | 7321 | CB | GLU I | 30 | 23.508 | −15.035 | −19.049 | 1.00 | 11.98 | C | C |
| ATOM | 7322 | CG | GLU I | 30 | 22.537 | −13.901 | −19.321 | 1.00 | 13.38 | C | C |
| ATOM | 7323 | CD | GLU I | 30 | 22.687 | −12.767 | −18.325 | 1.00 | 16.03 | C | C |
| ATOM | 7324 | OE1 | GLU I | 30 | 23.820 | −12.269 | −18.155 | 1.00 | 16.65 | C | O |
| ATOM | 7325 | OE2 | GLU I | 30 | 21.684 | −12.412 | −17.673 | 1.00 | 17.06 | C | O |
| ATOM | 7326 | C | GLU I | 30 | 23.814 | −16.480 | −21.047 | 1.00 | 11.39 | C | C |
| ATOM | 7327 | O | GLU I | 30 | 25.000 | −16.799 | −21.124 | 1.00 | 11.50 | C | O |
| ATOM | 7328 | N | GLU I | 31 | 23.036 | −16.344 | −22.111 | 1.00 | 11.11 | C | N |
| ATOM | 7329 | CA | GLU I | 31 | 23.626 | −16.233 | −23.427 | 1.00 | 11.06 | C | C |
| ATOM | 7330 | CB | GLU I | 31 | 23.376 | −17.494 | −24.251 | 1.00 | 11.48 | C | C |
| ATOM | 7331 | CG | GLU I | 31 | 22.395 | −17.322 | −25.391 | 1.00 | 13.90 | C | C |
| ATOM | 7332 | CD | GLU I | 31 | 22.329 | −18.546 | −26.280 | 1.00 | 17.27 | C | C |
| ATOM | 7333 | OE1 | GLU I | 31 | 22.658 | −18.428 | −27.478 | 1.00 | 17.55 | C | O |
| ATOM | 7334 | OE2 | GLU I | 31 | 21.999 | −19.638 | −25.769 | 1.00 | 18.73 | C | O |
| ATOM | 7335 | C | GLU I | 31 | 23.177 | −14.989 | −24.167 | 1.00 | 10.47 | C | C |
| ATOM | 7336 | O | GLU I | 31 | 22.010 | −14.597 | −24.118 | 1.00 | 10.12 | C | O |
| ATOM | 7337 | N | PHE I | 32 | 24.139 | −14.352 | −24.816 | 1.00 | 10.15 | C | N |
| ATOM | 7338 | CA | PHE I | 32 | 23.986 | −12.994 | −25.289 | 1.00 | 9.62 | C | C |
| ATOM | 7339 | CB | PHE I | 32 | 25.267 | −12.204 | −25.017 | 1.00 | 9.42 | C | C |
| ATOM | 7340 | CG | PHE I | 32 | 25.384 | −10.939 | −25.822 | 1.00 | 9.27 | C | C |
| ATOM | 7341 | CD1 | PHE I | 32 | 24.529 | −9.871 | −25.585 | 1.00 | 9.37 | C | C |
| ATOM | 7342 | CE1 | PHE I | 32 | 24.614 | −8.713 | −26.339 | 1.00 | 9.52 | C | C |
| ATOM | 7343 | CZ | PHE I | 32 | 25.594 | −8.593 | −27.306 | 1.00 | 9.82 | C | C |
| ATOM | 7344 | CE2 | PHE I | 32 | 26.458 | −9.651 | −27.548 | 1.00 | 10.13 | C | C |
| ATOM | 7345 | CD2 | PHE I | 32 | 26.372 | −10.802 | −26.786 | 1.00 | 10.06 | C | C |
| ATOM | 7346 | C | PHE I | 32 | 23.715 | −13.038 | −26.782 | 1.00 | 9.64 | C | C |
| ATOM | 7347 | O | PHE I | 32 | 24.336 | −13.811 | −27.512 | 1.00 | 9.84 | C | O |
| ATOM | 7348 | N | TYR I | 33 | 22.764 | −12.229 | −27.228 | 1.00 | 9.59 | C | N |
| ATOM | 7349 | CA | TYR I | 33 | 22.384 | −12.221 | −28.629 | 1.00 | 9.67 | C | C |
| ATOM | 7350 | CB | TYR I | 33 | 20.874 | −12.414 | −28.771 | 1.00 | 9.52 | C | C |
| ATOM | 7351 | CG | TYR I | 33 | 20.408 | −13.802 | −28.397 | 1.00 | 10.94 | C | C |
| ATOM | 7352 | CD1 | TYR I | 33 | 19.978 | −14.084 | −27.107 | 1.00 | 12.23 | C | C |
| ATOM | 7353 | CE1 | TYR I | 33 | 19.587 | −15.359 | −26.750 | 1.00 | 12.93 | C | C |
| ATOM | 7354 | CZ | TYR I | 33 | 19.675 | −16.381 | −27.670 | 1.00 | 13.47 | C | C |
| ATOM | 7355 | OH | TYR I | 33 | 19.297 | −17.656 | −27.315 | 1.00 | 14.54 | C | O |
| ATOM | 7356 | CE2 | TYR I | 33 | 20.151 | −16.137 | −28.941 | 1.00 | 13.56 | C | C |
| ATOM | 7357 | CD2 | TYR I | 33 | 20.538 | −14.858 | −29.288 | 1.00 | 12.96 | C | C |
| ATOM | 7358 | C | TYR I | 33 | 22.832 | −10.933 | −29.306 | 1.00 | 9.94 | C | C |
| ATOM | 7359 | O | TYR I | 33 | 22.138 | −9.917 | −29.257 | 1.00 | 10.16 | C | O |
| ATOM | 7360 | N | GLN I | 34 | 24.036 | −10.964 | −29.865 | 1.00 | 10.06 | C | N |
| ATOM | 7361 | CA | GLN I | 34 | 24.713 | −9.751 | −30.294 | 1.00 | 10.01 | C | C |
| ATOM | 7362 | CB | GLN I | 34 | 26.105 | −10.085 | −30.819 | 1.00 | 10.22 | C | C |
| ATOM | 7363 | CG | GLN I | 34 | 26.862 | −8.884 | −31.336 | 1.00 | 11.12 | C | C |
| ATOM | 7364 | CD | GLN I | 34 | 28.323 | −9.187 | −31.577 | 1.00 | 13.13 | C | C |
| ATOM | 7365 | OE1 | GLN I | 34 | 28.674 | −9.887 | −32.528 | 1.00 | 14.24 | C | O |
| ATOM | 7366 | NE2 | GLN I | 34 | 29.183 | −8.693 | −30.693 | 1.00 | 13.79 | C | N |
| ATOM | 7367 | C | GLN I | 34 | 23.914 | −9.028 | −31.370 | 1.00 | 9.94 | C | C |
| ATOM | 7368 | O | GLN I | 34 | 24.051 | −7.818 | −31.550 | 1.00 | 10.08 | C | O |
| ATOM | 7369 | N | SER I | 35 | 23.079 | −9.781 | −32.081 | 1.00 | 9.87 | C | N |
| ATOM | 7370 | CA | SER I | 35 | 22.336 | −9.255 | −33.225 | 1.00 | 9.91 | C | C |
| ATOM | 7371 | CB | SER I | 35 | 21.995 | −10.382 | −34.206 | 1.00 | 10.03 | C | C |
| ATOM | 7372 | OG | SER I | 35 | 21.241 | −11.403 | −33.574 | 1.00 | 10.81 | C | O |
| ATOM | 7373 | C | SER I | 35 | 21.059 | −8.555 | −32.775 | 1.00 | 9.56 | C | C |
| ATOM | 7374 | O | SER I | 35 | 20.248 | −8.124 | −33.595 | 1.00 | 9.59 | C | O |
| ATOM | 7375 | N | THR I | 36 | 20.823 | −8.581 | −31.472 | 1.00 | 9.21 | C | N |
| ATOM | 7376 | CA | THR I | 36 | 19.592 | −8.066 | −30.903 | 1.00 | 9.06 | C | C |
| ATOM | 7377 | CB | THR I | 36 | 18.648 | −9.214 | −30.517 | 1.00 | 9.49 | C | C |
| ATOM | 7378 | OG1 | THR I | 36 | 18.292 | −9.956 | −31.691 | 1.00 | 10.82 | C | O |
| ATOM | 7379 | CG2 | THR I | 36 | 17.392 | −8.673 | −29.860 | 1.00 | 9.45 | C | C |
| ATOM | 7380 | C | THR I | 36 | 19.966 | −7.274 | −29.661 | 1.00 | 8.77 | C | C |
| ATOM | 7381 | O | THR I | 36 | 19.112 | −6.915 | −28.848 | 1.00 | 8.60 | C | O |
| ATOM | 7382 | N | CYS I | 37 | 21.266 | −7.035 | −29.515 | 1.00 | 8.69 | C | N |
| ATOM | 7383 | CA | CYS I | 37 | 21.818 | −6.428 | −28.314 | 1.00 | 8.69 | C | C |
| ATOM | 7384 | CB | CYS I | 37 | 21.917 | −4.911 | −28.490 | 1.00 | 8.81 | C | C |
| ATOM | 7385 | SG | CYS I | 37 | 23.280 | −4.138 | −27.602 | 1.00 | 10.80 | C | S |
| ATOM | 7386 | C | CYS I | 37 | 20.954 | −6.758 | −27.107 | 1.00 | 8.27 | C | C |
| ATOM | 7387 | O | CYS I | 37 | 20.341 | −5.872 | −26.513 | 1.00 | 8.27 | C | O |
| ATOM | 7388 | N | SER I | 38 | 20.825 | −8.045 | −26.808 | 1.00 | 7.92 | C | N |
| ATOM | 7389 | CA | SER I | 38 | 19.990 | −8.476 | −25.699 | 1.00 | 7.51 | C | C |
| ATOM | 7390 | CB | SER I | 38 | 18.537 | −8.626 | −26.150 | 1.00 | 7.63 | C | C |
| ATOM | 7391 | OG | SER I | 38 | 18.429 | −9.583 | −27.190 | 1.00 | 8.54 | C | O |
| ATOM | 7392 | C | SER I | 38 | 20.496 | −9.784 | −25.110 | 1.00 | 7.02 | C | C |
| ATOM | 7393 | O | SER I | 38 | 21.196 | −10.546 | −25.776 | 1.00 | 6.83 | C | O |
| ATOM | 7394 | N | ALA I | 39 | 20.214 | −9.995 | −23.830 | 1.00 | 6.64 | C | N |
| ATOM | 7395 | CA | ALA I | 39 | 20.727 | −11.159 | −23.127 | 1.00 | 6.58 | C | C |
| ATOM | 7396 | CB | ALA I | 39 | 21.834 | −10.761 | −22.174 | 1.00 | 6.67 | C | C |
| ATOM | 7397 | C | ALA I | 39 | 19.615 | −11.885 | −22.388 | 1.00 | 6.93 | C | C |
| ATOM | 7398 | O | ALA I | 39 | 18.683 | −11.263 | −21.878 | 1.00 | 7.38 | C | O |
| ATOM | 7399 | N | VAL I | 40 | 19.675 | −13.211 | −22.415 | 1.00 | 6.87 | C | N |
| ATOM | 7400 | CA | VAL I | 40 | 18.707 | −14.034 | −21.706 | 1.00 | 6.55 | C | C |
| ATOM | 7401 | CB | VAL I | 40 | 17.905 | −14.928 | −22.668 | 1.00 | 6.22 | C | C |
| ATOM | 7402 | CG1 | VAL I | 40 | 17.196 | −16.031 | −21.899 | 1.00 | 6.52 | C | C |
| ATOM | 7403 | CG2 | VAL I | 40 | 16.905 | −14.098 | −23.456 | 1.00 | 6.62 | C | C |
| ATOM | 7404 | C | VAL I | 40 | 19.394 | −14.909 | −20.670 | 1.00 | 6.73 | C | C |
| ATOM | 7405 | O | VAL I | 40 | 20.351 | −15.620 | −20.977 | 1.00 | 6.83 | C | O |
| ATOM | 7406 | N | SER I | 41 | 18.916 | −14.822 | −19.434 | 1.00 | 6.86 | C | N |
| ATOM | 7407 | CA | SER I | 41 | 19.298 | −15.760 | −18.388 | 1.00 | 6.75 | C | C |
| ATOM | 7408 | CB | SER I | 41 | 19.221 | −15.082 | −17.022 | 1.00 | 7.06 | C | C |
| ATOM | 7409 | OG | SER I | 41 | 18.745 | −13.752 | −17.148 | 1.00 | 7.50 | C | O |
| ATOM | 7410 | C | SER I | 41 | 18.399 | −16.988 | −18.409 | 1.00 | 6.56 | C | C |
| ATOM | 7411 | O | SER I | 41 | 17.174 | −16.873 | −18.403 | 1.00 | 6.08 | C | O |
| ATOM | 7412 | N | LYS I | 42 | 19.019 | −18.163 | −18.409 | 1.00 | 7.02 | C | N |
| ATOM | 7413 | CA | LYS I | 42 | 18.311 | −19.409 | −18.665 | 1.00 | 7.83 | C | C |
| ATOM | 7414 | CB | LYS I | 42 | 18.835 | −20.066 | −19.943 | 1.00 | 8.15 | C | C |
| ATOM | 7415 | CG | LYS I | 42 | 18.753 | −19.192 | −21.182 | 1.00 | 9.75 | C | C |
| ATOM | 7416 | CD | LYS I | 42 | 19.416 | −19.872 | −22.369 | 1.00 | 12.62 | C | C |
| ATOM | 7417 | CE | LYS I | 42 | 19.087 | −19.162 | −23.671 | 1.00 | 14.04 | C | C |
| ATOM | 7418 | NZ | LYS I | 42 | 19.298 | −20.046 | −24.851 | 1.00 | 14.96 | C | N |
| ATOM | 7419 | C | LYS I | 42 | 18.489 | −20.367 | −17.495 | 1.00 | 7.80 | C | C |
| ATOM | 7420 | O | LYS I | 42 | 19.512 | −20.336 | −16.810 | 1.00 | 8.15 | C | O |
| ATOM | 7421 | N | GLY I | 43 | 17.581 | −21.330 | −17.389 | 1.00 | 7.59 | C | N |
| ATOM | 7422 | CA | GLY I | 43 | 17.783 | −22.472 | −16.508 | 1.00 | 7.35 | C | C |
| ATOM | 7423 | C | GLY I | 43 | 17.031 | −22.326 | −15.202 | 1.00 | 7.10 | C | C |
| ATOM | 7424 | O | GLY I | 43 | 17.428 | −22.883 | −14.179 | 1.00 | 7.13 | C | O |
| ATOM | 7425 | N | TYR I | 44 | 15.907 | −21.621 | −15.253 | 1.00 | 7.01 | C | N |
| ATOM | 7426 | CA | TYR I | 44 | 15.095 | −21.391 | −14.066 | 1.00 | 6.89 | C | C |
| ATOM | 7427 | CB | TYR I | 44 | 14.678 | −19.924 | −13.982 | 1.00 | 6.88 | C | C |
| ATOM | 7428 | CG | TYR I | 44 | 15.828 | −18.979 | −13.738 | 1.00 | 6.41 | C | C |
| ATOM | 7429 | CD1 | TYR I | 44 | 16.374 | −18.238 | −14.776 | 1.00 | 6.62 | C | C |
| ATOM | 7430 | CE1 | TYR I | 44 | 17.430 | −17.376 | −14.558 | 1.00 | 6.48 | C | C |
| ATOM | 7431 | CZ | TYR I | 44 | 17.946 | −17.240 | −13.287 | 1.00 | 6.88 | C | C |
| ATOM | 7432 | OH | TYR I | 44 | 18.989 | −16.371 | −13.062 | 1.00 | 7.50 | C | O |
| ATOM | 7433 | CE2 | TYR I | 44 | 17.438 | −17.984 | −12.245 | 1.00 | 6.68 | C | C |
| ATOM | 7434 | CD2 | TYR I | 44 | 16.388 | −18.851 | −12.475 | 1.00 | 5.91 | C | C |
| ATOM | 7435 | C | TYR I | 44 | 13.859 | −22.275 | −14.065 | 1.00 | 6.86 | C | C |
| ATOM | 7436 | O | TYR I | 44 | 13.320 | −22.602 | −15.122 | 1.00 | 7.14 | C | O |
| ATOM | 7437 | N | LEU I | 45 | 13.309 | −22.498 | −12.875 | 1.00 | 6.44 | C | N |
| ATOM | 7438 | CA | LEU I | 45 | 12.131 | −23.342 | −12.733 | 1.00 | 6.12 | C | C |
| ATOM | 7439 | CB | LEU I | 45 | 12.479 | −24.650 | −12.019 | 1.00 | 6.31 | C | C |
| ATOM | 7440 | CG | LEU I | 45 | 13.457 | −25.536 | −12.798 | 1.00 | 6.38 | C | C |
| ATOM | 7441 | CD1 | LEU I | 45 | 14.250 | −26.452 | −11.877 | 1.00 | 7.05 | C | C |
| ATOM | 7442 | CD2 | LEU I | 45 | 12.736 | −26.332 | −13.874 | 1.00 | 7.05 | C | C |
| ATOM | 7443 | C | LEU I | 45 | 10.924 | −22.641 | −12.096 | 1.00 | 5.84 | C | C |
| ATOM | 7444 | O | LEU I | 45 | 10.954 | −22.268 | −10.921 | 1.00 | 5.94 | C | O |
| ATOM | 7445 | N | SER I | 46 | 9.806 | −22.689 | −12.821 | 1.00 | 5.43 | C | N |
| ATOM | 7446 | CA | SER I | 46 | 8.639 | −21.822 | −12.611 | 1.00 | 5.26 | C | C |
| ATOM | 7447 | CB | SER I | 46 | 7.803 | −21.796 | −13.891 | 1.00 | 5.21 | C | C |
| ATOM | 7448 | OG | SER I | 46 | 7.441 | −23.112 | −14.280 | 1.00 | 4.95 | C | O |
| ATOM | 7449 | C | SER I | 46 | 7.793 | −22.404 | −11.485 | 1.00 | 5.51 | C | C |
| ATOM | 7450 | O | SER I | 46 | 8.086 | −23.500 | −11.013 | 1.00 | 6.03 | C | O |
| ATOM | 7451 | N | ALA I | 47 | 6.787 | −21.679 | −11.001 | 1.00 | 5.24 | C | N |
| ATOM | 7452 | CA | ALA I | 47 | 6.265 | −22.042 | −9.688 | 1.00 | 4.83 | C | C |
| ATOM | 7453 | CB | ALA I | 47 | 7.391 | −22.094 | −8.675 | 1.00 | 5.01 | C | C |
| ATOM | 7454 | C | ALA I | 47 | 5.061 | −21.315 | −9.106 | 1.00 | 4.35 | C | C |
| ATOM | 7455 | O | ALA I | 47 | 5.100 | −20.099 | −8.920 | 1.00 | 4.45 | C | O |
| ATOM | 7456 | N | LEU I | 48 | 4.246 | −22.119 | −8.428 | 1.00 | 3.67 | C | N |
| ATOM | 7457 | CA | LEU I | 48 | 3.120 | −21.630 | −7.645 | 1.00 | 3.15 | C | C |
| ATOM | 7458 | CB | LEU I | 48 | 1.802 | −21.948 | −8.359 | 1.00 | 2.78 | C | C |
| ATOM | 7459 | CG | LEU I | 48 | 1.785 | −21.864 | −9.889 | 1.00 | 2.18 | C | C |
| ATOM | 7460 | CD1 | LEU I | 48 | 0.350 | −21.834 | −10.383 | 1.00 | 2.00 | C | C |
| ATOM | 7461 | CD2 | LEU I | 48 | 2.544 | −20.643 | −10.386 | 1.00 | 3.94 | C | C |
| ATOM | 7462 | C | LEU I | 48 | 3.128 | −22.240 | −6.239 | 1.00 | 3.06 | C | C |
| ATOM | 7463 | O | LEU I | 48 | 3.433 | −23.422 | −6.065 | 1.00 | 3.27 | C | O |
| ATOM | 7464 | N | ARG I | 49 | 2.926 | −21.394 | −5.233 | 1.00 | 2.91 | C | N |
| ATOM | 7465 | CA | ARG I | 49 | 2.903 | −21.846 | −3.846 | 1.00 | 2.88 | C | C |
| ATOM | 7466 | CB | ARG I | 49 | 3.036 | −20.658 | −2.887 | 1.00 | 2.69 | C | C |
| ATOM | 7467 | CG | ARG I | 49 | 3.904 | −20.942 | −1.663 | 1.00 | 2.15 | C | C |
| ATOM | 7468 | CD | ARG I | 49 | 3.321 | −20.322 | −0.404 | 1.00 | 2.00 | C | C |
| ATOM | 7469 | NE | ARG I | 49 | 2.284 | −21.163 | 0.190 | 1.00 | 5.18 | C | N |
| ATOM | 7470 | CZ | ARG I | 49 | 1.050 | −20.748 | 0.463 | 1.00 | 7.68 | C | C |
| ATOM | 7471 | NH1 | ARG I | 49 | 0.699 | −19.492 | 0.217 | 1.00 | 8.32 | C | N |
| ATOM | 7472 | NH2 | ARG I | 49 | 0.181 | −21.570 | 1.036 | 1.00 | 8.12 | C | N |
| ATOM | 7473 | C | ARG I | 49 | 1.625 | −22.619 | −3.544 | 1.00 | 3.09 | C | C |
| ATOM | 7474 | O | ARG I | 49 | 0.527 | −22.170 | −3.873 | 1.00 | 3.08 | C | O |
| ATOM | 7475 | N | THR I | 50 | 1.774 | −23.803 | −2.956 | 1.00 | 3.37 | C | N |
| ATOM | 7476 | CA | THR I | 50 | 0.625 | −24.551 | −2.456 | 1.00 | 3.55 | C | C |
| ATOM | 7477 | CB | THR I | 50 | 0.042 | −25.503 | −3.512 | 1.00 | 3.25 | C | C |
| ATOM | 7478 | OG1 | THR I | 50 | 1.091 | −26.292 | −4.086 | 1.00 | 2.66 | C | O |
| ATOM | 7479 | CG2 | THR I | 50 | −0.670 | −24.723 | −4.605 | 1.00 | 3.46 | C | C |
| ATOM | 7480 | C | THR I | 50 | 0.919 | −25.349 | −1.195 | 1.00 | 4.07 | C | C |
| ATOM | 7481 | O | THR I | 50 | 0.137 | −25.308 | −0.247 | 1.00 | 4.35 | C | O |
| ATOM | 7482 | N | GLY I | 51 | 1.892 | −26.248 | −1.274 | 1.00 | 4.24 | C | N |
| ATOM | 7483 | CA | GLY I | 51 | 2.393 | −26.864 | −0.060 | 1.00 | 4.78 | C | C |
| ATOM | 7484 | C | GLY I | 51 | 2.414 | −25.769 | 0.981 | 1.00 | 4.98 | C | C |
| ATOM | 7485 | O | GLY I | 51 | 3.082 | −24.754 | 0.786 | 1.00 | 5.00 | C | O |
| ATOM | 7486 | N | TRP I | 52 | 1.481 | −25.829 | 1.923 | 1.00 | 5.34 | C | N |
| ATOM | 7487 | CA | TRP I | 52 | 1.440 | −24.823 | 2.974 | 1.00 | 5.42 | C | C |
| ATOM | 7488 | CB | TRP I | 52 | 0.124 | −24.048 | 2.951 | 1.00 | 5.75 | C | C |
| ATOM | 7489 | CG | TRP I | 52 | −0.090 | −23.284 | 1.696 | 1.00 | 6.38 | C | C |
| ATOM | 7490 | CD1 | TRP I | 52 | −1.020 | −23.544 | 0.734 | 1.00 | 7.27 | C | C |
| ATOM | 7491 | NE1 | TRP I | 52 | −0.899 | −22.647 | −0.297 | 1.00 | 7.46 | C | N |
| ATOM | 7492 | CE2 | TRP I | 52 | 0.129 | −21.785 | −0.018 | 1.00 | 7.15 | C | C |
| ATOM | 7493 | CD2 | TRP I | 52 | 0.671 | −22.164 | 1.228 | 1.00 | 7.02 | C | C |
| ATOM | 7494 | CE3 | TRP I | 52 | 1.741 | −21.428 | 1.752 | 1.00 | 6.83 | C | C |
| ATOM | 7495 | CZ3 | TRP I | 52 | 2.221 | −20.347 | 1.027 | 1.00 | 6.73 | C | C |
| ATOM | 7496 | CH2 | TRP I | 52 | 1.664 | −20.000 | −0.214 | 1.00 | 7.13 | C | C |
| ATOM | 7497 | CZ2 | TRP I | 52 | 0.621 | −20.706 | −0.751 | 1.00 | 6.82 | C | C |
| ATOM | 7498 | C | TRP I | 52 | 1.680 | −25.408 | 4.354 | 1.00 | 5.34 | C | C |
| ATOM | 7499 | O | TRP I | 52 | 1.222 | −26.507 | 4.667 | 1.00 | 5.47 | C | O |
| ATOM | 7500 | N | TYR I | 53 | 2.237 | −24.581 | 5.227 | 1.00 | 5.29 | C | N |
| ATOM | 7501 | CA | TYR I | 53 | 2.492 | −24.977 | 6.598 | 1.00 | 5.58 | C | C |
| ATOM | 7502 | CB | TYR I | 53 | 4.002 | −25.087 | 6.829 | 1.00 | 5.51 | C | C |
| ATOM | 7503 | CG | TYR I | 53 | 4.401 | −25.463 | 8.239 | 1.00 | 6.59 | C | C |
| ATOM | 7504 | CD1 | TYR I | 53 | 4.613 | −26.791 | 8.591 | 1.00 | 8.25 | C | C |
| ATOM | 7505 | CE1 | TYR I | 53 | 5.010 | −27.136 | 9.872 | 1.00 | 9.40 | C | C |
| ATOM | 7506 | CZ | TYR I | 53 | 5.280 | −26.145 | 10.790 | 1.00 | 9.24 | C | C |
| ATOM | 7507 | OH | TYR I | 53 | 5.693 | −26.480 | 12.058 | 1.00 | 9.61 | C | O |
| ATOM | 7508 | CE2 | TYR I | 53 | 5.162 | −24.818 | 10.437 | 1.00 | 8.78 | C | C |
| ATOM | 7509 | CD2 | TYR I | 53 | 4.758 | −24.485 | 9.157 | 1.00 | 7.93 | C | C |
| ATOM | 7510 | C | TYR I | 53 | 1.869 | −23.950 | 7.539 | 1.00 | 5.74 | C | C |
| ATOM | 7511 | O | TYR I | 53 | 2.281 | −22.790 | 7.558 | 1.00 | 6.19 | C | O |
| ATOM | 7512 | N | THR I | 54 | 0.756 | −24.328 | 8.159 | 1.00 | 6.04 | C | N |
| ATOM | 7513 | CA | THR I | 54 | 0.075 | −23.456 | 9.109 | 1.00 | 6.67 | C | C |
| ATOM | 7514 | CB | THR I | 54 | −1.298 | −24.028 | 9.513 | 1.00 | 6.97 | C | C |
| ATOM | 7515 | OG1 | THR I | 54 | −2.247 | −23.782 | 8.467 | 1.00 | 7.51 | C | O |
| ATOM | 7516 | CG2 | THR I | 54 | −1.790 | −23.384 | 10.800 | 1.00 | 7.98 | C | C |
| ATOM | 7517 | C | THR I | 54 | 0.928 | −23.266 | 10.359 | 1.00 | 6.97 | C | C |
| ATOM | 7518 | O | THR I | 54 | 1.713 | −24.141 | 10.723 | 1.00 | 7.32 | C | O |
| ATOM | 7519 | N | SER I | 55 | 0.775 | −22.120 | 11.013 | 1.00 | 7.24 | C | N |
| ATOM | 7520 | CA | SER I | 55 | 1.085 | −22.019 | 12.433 | 1.00 | 7.65 | C | C |
| ATOM | 7521 | CB | SER I | 55 | 2.589 | −21.862 | 12.654 | 1.00 | 7.69 | C | C |
| ATOM | 7522 | OG | SER I | 55 | 2.868 | −21.507 | 13.998 | 1.00 | 7.51 | C | O |
| ATOM | 7523 | C | SER I | 55 | 0.338 | −20.878 | 13.104 | 1.00 | 8.03 | C | C |
| ATOM | 7524 | O | SER I | 55 | 0.238 | −19.778 | 12.560 | 1.00 | 8.43 | C | O |
| ATOM | 7525 | N | VAL I | 56 | −0.094 | −21.121 | 14.335 | 1.00 | 8.14 | C | N |
| ATOM | 7526 | CA | VAL I | 56 | −1.029 | −20.234 | 15.006 | 1.00 | 8.41 | C | C |
| ATOM | 7527 | CB | VAL I | 56 | −2.209 | −21.014 | 15.598 | 1.00 | 8.72 | C | C |
| ATOM | 7528 | CG1 | VAL I | 56 | −3.403 | −20.094 | 15.787 | 1.00 | 9.29 | C | C |
| ATOM | 7529 | CG2 | VAL I | 56 | −2.569 | −22.184 | 14.695 | 1.00 | 9.51 | C | C |
| ATOM | 7530 | C | VAL I | 56 | −0.331 | −19.478 | 16.123 | 1.00 | 8.19 | C | C |
| ATOM | 7531 | O | VAL I | 56 | 0.075 | −20.066 | 17.122 | 1.00 | 8.54 | C | O |
| ATOM | 7532 | N | ILE I | 57 | −0.187 | −18.171 | 15.943 | 1.00 | 7.91 | C | N |
| ATOM | 7533 | CA | ILE I | 57 | 0.639 | −17.368 | 16.832 | 1.00 | 7.64 | C | C |
| ATOM | 7534 | CB | ILE I | 57 | 1.675 | −16.539 | 16.051 | 1.00 | 7.35 | C | C |
| ATOM | 7535 | CG1 | ILE I | 57 | 2.612 | −17.461 | 15.262 | 1.00 | 7.25 | C | C |
| ATOM | 7536 | CD1 | ILE I | 57 | 3.526 | −16.730 | 14.294 | 1.00 | 8.42 | C | C |
| ATOM | 7537 | CG2 | ILE I | 57 | 2.457 | −15.632 | 16.993 | 1.00 | 7.52 | C | C |
| ATOM | 7538 | C | ILE I | 57 | −0.222 | −16.445 | 17.680 | 1.00 | 7.69 | C | C |
| ATOM | 7539 | O | ILE I | 57 | −1.150 | −15.810 | 17.178 | 1.00 | 7.79 | C | O |
| ATOM | 7540 | N | THR I | 58 | −0.024 | −16.523 | 18.989 | 1.00 | 7.85 | C | N |
| ATOM | 7541 | CA | THR I | 58 | −0.929 | −15.900 | 19.938 | 1.00 | 8.15 | C | C |
| ATOM | 7542 | CB | THR I | 58 | −1.660 | −16.954 | 20.777 | 1.00 | 8.14 | C | C |
| ATOM | 7543 | OG1 | THR I | 58 | −1.584 | −18.225 | 20.120 | 1.00 | 8.62 | C | O |
| ATOM | 7544 | CG2 | THR I | 58 | −3.116 | −16.566 | 20.957 | 1.00 | 7.94 | C | C |
| ATOM | 7545 | C | THR I | 58 | −0.152 | −14.983 | 20.869 | 1.00 | 8.20 | C | C |
| ATOM | 7546 | O | THR I | 58 | 0.808 | −15.408 | 21.510 | 1.00 | 8.18 | C | O |
| ATOM | 7547 | N | ILE I | 59 | −0.539 | −13.713 | 20.903 | 1.00 | 8.54 | C | N |
| ATOM | 7548 | CA | ILE I | 59 | 0.226 | −12.694 | 21.616 | 1.00 | 9.23 | C | C |
| ATOM | 7549 | CB | ILE I | 59 | 0.863 | −11.690 | 20.634 | 1.00 | 8.92 | C | C |
| ATOM | 7550 | CG1 | ILE I | 59 | 1.868 | −12.391 | 19.717 | 1.00 | 9.27 | C | C |
| ATOM | 7551 | CD1 | ILE I | 59 | 2.587 | −11.453 | 18.770 | 1.00 | 10.89 | C | C |
| ATOM | 7552 | CG2 | ILE I | 59 | 1.516 | −10.544 | 21.390 | 1.00 | 8.74 | C | C |
| ATOM | 7553 | C | ILE I | 59 | −0.691 | −11.920 | 22.553 | 1.00 | 10.02 | C | C |
| ATOM | 7554 | O | ILE I | 59 | −1.692 | −11.357 | 22.113 | 1.00 | 10.20 | C | O |
| ATOM | 7555 | N | GLU I | 60 | −0.439 | −12.007 | 23.853 | 1.00 | 10.95 | C | N |
| ATOM | 7556 | CA | GLU I | 60 | −1.462 | −11.602 | 24.808 | 1.00 | 12.13 | C | C |
| ATOM | 7557 | CB | GLU I | 60 | −0.963 | −11.829 | 26.248 | 1.00 | 12.44 | C | C |
| ATOM | 7558 | CG | GLU I | 60 | −0.554 | −13.288 | 26.547 | 1.00 | 15.04 | C | C |
| ATOM | 7559 | CD | GLU I | 60 | −0.560 | −13.630 | 28.036 | 1.00 | 19.34 | C | C |
| ATOM | 7560 | OE1 | GLU I | 60 | 0.413 | −13.267 | 28.733 | 1.00 | 20.63 | C | O |
| ATOM | 7561 | OE2 | GLU I | 60 | −1.469 | −14.368 | 28.478 | 1.00 | 21.19 | C | O |
| ATOM | 7562 | C | GLU I | 60 | −1.856 | −10.131 | 24.547 | 1.00 | 12.46 | C | C |
| ATOM | 7563 | O | GLU I | 60 | −0.984 | −9.259 | 24.567 | 1.00 | 12.68 | C | O |
| ATOM | 7564 | N | LEU I | 61 | −3.138 | −9.818 | 24.298 | 1.00 | 12.80 | C | N |
| ATOM | 7565 | CA | LEU I | 61 | −3.535 | −8.473 | 24.718 | 1.00 | 12.71 | C | C |
| ATOM | 7566 | CB | LEU I | 61 | −5.021 | −8.120 | 24.699 | 1.00 | 12.55 | C | C |
| ATOM | 7567 | CG | LEU I | 61 | −5.052 | −6.753 | 25.421 | 1.00 | 12.39 | C | C |
| ATOM | 7568 | CD1 | LEU I | 61 | −4.358 | −5.650 | 24.625 | 1.00 | 13.14 | C | C |
| ATOM | 7569 | CD2 | LEU I | 61 | −6.421 | −6.318 | 25.899 | 1.00 | 12.50 | C | C |
| ATOM | 7570 | C | LEU I | 61 | −3.097 | −8.585 | 26.133 | 1.00 | 12.94 | C | C |
| ATOM | 7571 | O | LEU I | 61 | −3.578 | −9.474 | 26.843 | 1.00 | 12.78 | C | O |
| ATOM | 7572 | N | SER I | 62 | −1.846 | −8.190 | 26.260 | 1.00 | 13.61 | C | N |
| ATOM | 7573 | CA | SER I | 62 | −1.183 | −8.231 | 27.527 | 1.00 | 14.70 | C | C |
| ATOM | 7574 | CB | SER I | 62 | 0.326 | −8.252 | 27.325 | 1.00 | 14.52 | C | C |
| ATOM | 7575 | OG | SER I | 62 | 0.724 | −9.440 | 26.664 | 1.00 | 14.80 | C | O |
| ATOM | 7576 | C | SER I | 62 | −1.605 | −6.983 | 28.261 | 1.00 | 15.48 | C | C |
| ATOM | 7577 | O | SER I | 62 | −1.206 | −5.880 | 27.893 | 1.00 | 15.50 | C | O |
| ATOM | 7578 | N | ASN I | 63 | −2.602 | −7.135 | 29.121 | 1.00 | 16.61 | C | N |
| ATOM | 7579 | CA | ASN I | 63 | −3.232 | −5.978 | 29.716 | 1.00 | 17.77 | C | C |
| ATOM | 7580 | CB | ASN I | 63 | −4.616 | −6.314 | 30.263 | 1.00 | 17.73 | C | C |
| ATOM | 7581 | CG | ASN I | 63 | −5.566 | −5.142 | 30.175 | 1.00 | 18.52 | C | C |
| ATOM | 7582 | OD1 | ASN I | 63 | −5.140 | −3.987 | 30.185 | 1.00 | 18.29 | C | O |
| ATOM | 7583 | ND2 | ASN I | 63 | −6.837 | −5.433 | 29.933 | 1.00 | 19.40 | C | N |
| ATOM | 7584 | C | ASN I | 63 | −2.369 | −5.335 | 30.787 | 1.00 | 18.53 | C | C |
| ATOM | 7585 | O | ASN I | 63 | −1.589 | −6.007 | 31.463 | 1.00 | 18.82 | C | O |
| ATOM | 7586 | N | ILE I | 64 | −2.403 | −4.010 | 30.819 | 1.00 | 19.60 | C | N |
| ATOM | 7587 | CA | ILE I | 64 | −1.812 | −3.252 | 31.908 | 1.00 | 21.04 | C | C |
| ATOM | 7588 | CB | ILE I | 64 | −1.433 | −1.836 | 31.448 | 1.00 | 20.60 | C | C |
| ATOM | 7589 | CG1 | ILE I | 64 | −0.845 | −1.878 | 30.038 | 1.00 | 20.13 | C | C |
| ATOM | 7590 | CD1 | ILE I | 64 | −0.383 | −0.534 | 29.522 | 1.00 | 20.19 | C | C |
| ATOM | 7591 | CG2 | ILE I | 64 | −0.451 | −1.211 | 32.416 | 1.00 | 20.74 | C | C |
| ATOM | 7592 | C | ILE I | 64 | −2.778 | −3.148 | 33.082 | 1.00 | 22.58 | C | C |
| ATOM | 7593 | O | ILE I | 64 | −3.951 | −2.832 | 32.900 | 1.00 | 23.07 | C | O |
| ATOM | 7594 | N | LYS I | 65 | −2.259 | −3.322 | 34.292 | 1.00 | 24.21 | C | N |
| ATOM | 7595 | CA | LYS I | 65 | −2.904 | −2.779 | 35.482 | 1.00 | 25.86 | C | C |
| ATOM | 7596 | CB | LYS I | 65 | −2.929 | −3.822 | 36.602 | 1.00 | 25.90 | C | C |
| ATOM | 7597 | CG | LYS I | 65 | −2.083 | −5.056 | 36.323 | 1.00 | 26.69 | C | C |
| ATOM | 7598 | CD | LYS I | 65 | −0.616 | −4.698 | 36.160 | 1.00 | 28.26 | C | C |
| ATOM | 7599 | CE | LYS I | 65 | 0.273 | −5.606 | 36.992 | 1.00 | 28.67 | C | C |
| ATOM | 7600 | NZ | LYS I | 65 | 1.600 | −4.983 | 37.247 | 1.00 | 28.95 | C | N |
| ATOM | 7601 | C | LYS I | 65 | −2.215 | −1.500 | 35.961 | 1.00 | 26.81 | C | C |
| ATOM | 7602 | O | LYS I | 65 | −1.160 | −1.557 | 36.591 | 1.00 | 26.72 | C | O |
| ATOM | 7603 | N | GLU I | 66 | −2.903 | −0.371 | 35.798 | 1.00 | 27.90 | C | N |
| ATOM | 7604 | CA | GLU I | 66 | −2.291 | 0.962 | 35.878 | 1.00 | 28.82 | C | C |
| ATOM | 7605 | CB | GLU I | 66 | −3.184 | 1.983 | 35.163 | 1.00 | 28.88 | C | C |
| ATOM | 7606 | CG | GLU I | 66 | −3.354 | 3.309 | 35.901 | 1.00 | 30.33 | C | C |
| ATOM | 7607 | CD | GLU I | 66 | −4.570 | 4.084 | 35.431 | 1.00 | 33.17 | C | C |
| ATOM | 7608 | OE1 | GLU I | 66 | −4.590 | 5.323 | 35.595 | 1.00 | 35.05 | C | O |
| ATOM | 7609 | OE2 | GLU I | 66 | −5.488 | 3.457 | 34.860 | 1.00 | 33.14 | C | O |
| ATOM | 7610 | C | GLU I | 66 | −2.102 | 1.393 | 37.333 | 1.00 | 29.12 | C | C |
| ATOM | 7611 | O | GLU I | 66 | −3.002 | 1.204 | 38.148 | 1.00 | 29.28 | C | O |
| ATOM | 7612 | N | ASN I | 67 | −0.975 | 2.029 | 37.653 | 1.00 | 29.32 | C | N |
| ATOM | 7613 | CA | ASN I | 67 | −0.630 | 2.266 | 39.065 | 1.00 | 29.39 | C | C |
| ATOM | 7614 | CB | ASN I | 67 | 0.664 | 1.550 | 39.473 | 1.00 | 29.62 | C | C |
| ATOM | 7615 | CG | ASN I | 67 | 0.399 | 0.206 | 40.133 | 1.00 | 29.84 | C | C |
| ATOM | 7616 | OD1 | ASN I | 67 | −0.154 | 0.147 | 41.233 | 1.00 | 29.93 | C | O |
| ATOM | 7617 | ND2 | ASN I | 67 | 0.655 | −0.875 | 39.400 | 1.00 | 29.22 | C | N |
| ATOM | 7618 | C | ASN I | 67 | −0.670 | 3.712 | 39.571 | 1.00 | 29.15 | C | C |
| ATOM | 7619 | O | ASN I | 67 | 0.352 | 4.281 | 39.962 | 1.00 | 29.06 | C | O |
| ATOM | 7620 | N | LYS I | 68 | −1.872 | 4.284 | 39.584 | 1.00 | 28.87 | C | N |
| ATOM | 7621 | CA | LYS I | 68 | −2.302 | 5.212 | 40.630 | 1.00 | 28.78 | C | C |
| ATOM | 7622 | CB | LYS I | 68 | −3.436 | 4.596 | 41.454 | 1.00 | 28.89 | C | C |
| ATOM | 7623 | CG | LYS I | 68 | −4.461 | 3.838 | 40.617 | 1.00 | 29.26 | C | C |
| ATOM | 7624 | CD | LYS I | 68 | −5.357 | 2.964 | 41.483 | 1.00 | 29.68 | C | C |
| ATOM | 7625 | CE | LYS I | 68 | −5.626 | 3.615 | 42.835 | 1.00 | 29.46 | C | C |
| ATOM | 7626 | NZ | LYS I | 68 | −6.764 | 2.972 | 43.555 | 1.00 | 29.58 | C | N |
| ATOM | 7627 | C | LYS I | 68 | −1.171 | 5.722 | 41.535 | 1.00 | 28.60 | C | C |
| ATOM | 7628 | O | LYS I | 68 | −1.082 | 5.347 | 42.703 | 1.00 | 28.64 | C | O |
| ATOM | 7629 | N | CYS I | 69 | −0.274 | 6.525 | 40.961 | 1.00 | 28.09 | C | N |
| ATOM | 7630 | CA | CYS I | 69 | 0.585 | 7.455 | 41.715 | 1.00 | 27.63 | C | C |
| ATOM | 7631 | CB | CYS I | 69 | 2.068 | 7.270 | 41.326 | 1.00 | 27.07 | C | C |
| ATOM | 7632 | SG | CYS I | 69 | 2.742 | 8.433 | 40.058 | 1.00 | 28.01 | C | S |
| ATOM | 7633 | C | CYS I | 69 | 0.143 | 8.893 | 41.433 | 1.00 | 27.69 | C | C |
| ATOM | 7634 | O | CYS I | 69 | −0.588 | 9.134 | 40.473 | 1.00 | 27.41 | C | O |
| ATOM | 7635 | N | ASN I | 70 | 0.615 | 9.855 | 42.226 | 1.00 | 28.06 | C | N |
| ATOM | 7636 | CA | ASN I | 70 | 0.603 | 11.259 | 41.794 | 1.00 | 28.87 | C | C |
| ATOM | 7637 | CB | ASN I | 70 | −0.364 | 12.119 | 42.626 | 1.00 | 30.52 | C | C |
| ATOM | 7638 | CG | ASN I | 70 | −1.286 | 11.299 | 43.518 | 1.00 | 39.60 | C | C |
| ATOM | 7639 | OD1 | ASN I | 70 | −2.181 | 10.602 | 43.040 | 1.00 | 44.97 | C | O |
| ATOM | 7640 | ND2 | ASN I | 70 | −1.073 | 11.402 | 44.831 | 1.00 | 51.04 | C | N |
| ATOM | 7641 | C | ASN I | 70 | 1.978 | 11.934 | 41.727 | 1.00 | 26.97 | C | C |
| ATOM | 7642 | O | ASN I | 70 | 2.563 | 12.257 | 42.761 | 1.00 | 26.50 | C | O |
| ATOM | 7643 | N | GLY I | 71 | 2.371 | 12.349 | 40.523 | 1.00 | 25.46 | C | N |
| ATOM | 7644 | CA | GLY I | 71 | 3.643 | 13.052 | 40.316 | 1.00 | 23.67 | C | C |
| ATOM | 7645 | C | GLY I | 71 | 3.501 | 14.565 | 40.288 | 1.00 | 22.81 | C | C |
| ATOM | 7646 | O | GLY I | 71 | 2.415 | 15.094 | 40.516 | 1.00 | 22.79 | C | O |
| ATOM | 7647 | N | THR I | 72 | 4.585 | 15.268 | 39.975 | 1.00 | 21.93 | C | N |
| ATOM | 7648 | CA | THR I | 72 | 4.526 | 16.721 | 39.835 | 1.00 | 21.30 | C | C |
| ATOM | 7649 | CB | THR I | 72 | 5.923 | 17.363 | 39.940 | 1.00 | 21.12 | C | C |
| ATOM | 7650 | OG1 | THR I | 72 | 6.624 | 17.194 | 38.702 | 1.00 | 19.87 | C | O |
| ATOM | 7651 | CG2 | THR I | 72 | 6.725 | 16.723 | 41.063 | 1.00 | 20.76 | C | C |
| ATOM | 7652 | C | THR I | 72 | 3.864 | 17.132 | 38.518 | 1.00 | 21.50 | C | C |
| ATOM | 7653 | O | THR I | 72 | 4.384 | 16.852 | 37.438 | 1.00 | 21.85 | C | O |
| ATOM | 7654 | N | ASP I | 73 | 2.703 | 17.774 | 38.617 | 1.00 | 21.43 | C | N |
| ATOM | 7655 | CA | ASP I | 73 | 1.767 | 17.891 | 37.494 | 1.00 | 21.46 | C | C |
| ATOM | 7656 | CB | ASP I | 73 | 2.361 | 18.743 | 36.368 | 1.00 | 21.69 | C | C |
| ATOM | 7657 | CG | ASP I | 73 | 1.457 | 18.812 | 35.149 | 1.00 | 22.46 | C | C |
| ATOM | 7658 | OD1 | ASP I | 73 | 0.542 | 17.969 | 35.035 | 1.00 | 23.01 | C | O |
| ATOM | 7659 | OD2 | ASP I | 73 | 1.682 | 19.690 | 34.289 | 1.00 | 23.09 | C | O |
| ATOM | 7660 | C | ASP I | 73 | 1.323 | 16.536 | 36.950 | 1.00 | 20.96 | C | C |
| ATOM | 7661 | O | ASP I | 73 | 2.128 | 15.792 | 36.390 | 1.00 | 20.61 | C | O |
| ATOM | 7662 | N | ALA I | 74 | 0.019 | 16.285 | 36.989 | 1.00 | 20.42 | C | N |
| ATOM | 7663 | CA | ALA I | 74 | −0.512 | 14.977 | 36.622 | 1.00 | 20.38 | C | C |
| ATOM | 7664 | CB | ALA I | 74 | −1.027 | 14.248 | 37.855 | 1.00 | 20.11 | C | C |
| ATOM | 7665 | C | ALA I | 74 | −1.602 | 15.058 | 35.556 | 1.00 | 20.45 | C | C |
| ATOM | 7666 | O | ALA I | 74 | −2.490 | 14.208 | 35.509 | 1.00 | 20.46 | C | O |
| ATOM | 7667 | N | LYS I | 75 | −1.583 | 16.114 | 34.750 | 1.00 | 20.67 | C | N |
| ATOM | 7668 | CA | LYS I | 75 | −2.473 | 16.188 | 33.596 | 1.00 | 20.99 | C | C |
| ATOM | 7669 | CB | LYS I | 75 | −2.421 | 17.583 | 32.970 | 1.00 | 21.20 | C | C |
| ATOM | 7670 | CG | LYS I | 75 | −2.967 | 18.684 | 33.865 | 1.00 | 22.02 | C | C |
| ATOM | 7671 | CD | LYS I | 75 | −1.927 | 19.766 | 34.106 | 1.00 | 23.66 | C | C |
| ATOM | 7672 | CE | LYS I | 75 | −1.971 | 20.263 | 35.542 | 1.00 | 24.93 | C | C |
| ATOM | 7673 | NZ | LYS I | 75 | −0.622 | 20.658 | 36.036 | 1.00 | 25.55 | C | N |
| ATOM | 7674 | C | LYS I | 75 | −2.025 | 15.144 | 32.587 | 1.00 | 20.92 | C | C |
| ATOM | 7675 | O | LYS I | 75 | −2.801 | 14.680 | 31.751 | 1.00 | 20.92 | C | O |
| ATOM | 7676 | N | VAL I | 76 | −0.731 | 14.853 | 32.634 | 1.00 | 20.63 | C | N |
| ATOM | 7677 | CA | VAL I | 76 | −0.147 | 13.700 | 31.971 | 1.00 | 20.36 | C | C |
| ATOM | 7678 | CB | VAL I | 76 | 1.386 | 13.792 | 32.017 | 1.00 | 20.25 | C | C |
| ATOM | 7679 | CG1 | VAL I | 76 | 2.009 | 12.475 | 31.612 | 1.00 | 20.82 | C | C |
| ATOM | 7680 | CG2 | VAL I | 76 | 1.873 | 14.923 | 31.130 | 1.00 | 20.09 | C | C |
| ATOM | 7681 | C | VAL I | 76 | −0.586 | 12.393 | 32.629 | 1.00 | 20.29 | C | C |
| ATOM | 7682 | O | VAL I | 76 | −0.452 | 12.225 | 33.840 | 1.00 | 20.21 | C | O |
| ATOM | 7683 | N | LYS I | 77 | −1.106 | 11.471 | 31.826 | 1.00 | 20.17 | C | N |
| ATOM | 7684 | CA | LYS I | 77 | −1.642 | 10.218 | 32.348 | 1.00 | 20.17 | C | C |
| ATOM | 7685 | CB | LYS I | 77 | −3.103 | 10.393 | 32.760 | 1.00 | 20.62 | C | C |
| ATOM | 7686 | CG | LYS I | 77 | −3.297 | 10.767 | 34.215 | 1.00 | 23.31 | C | C |
| ATOM | 7687 | CD | LYS I | 77 | −4.508 | 11.664 | 34.388 | 1.00 | 26.74 | C | C |
| ATOM | 7688 | CE | LYS I | 77 | −4.529 | 12.291 | 35.767 | 1.00 | 28.32 | C | C |
| ATOM | 7689 | NZ | LYS I | 77 | −5.869 | 12.836 | 36.105 | 1.00 | 29.66 | C | N |
| ATOM | 7690 | C | LYS I | 77 | −1.523 | 9.105 | 31.317 | 1.00 | 19.45 | C | C |
| ATOM | 7691 | O | LYS I | 77 | −2.517 | 8.496 | 30.921 | 1.00 | 19.36 | C | O |
| ATOM | 7692 | N | LEU I | 78 | −0.297 | 8.863 | 30.871 | 1.00 | 18.70 | C | N |
| ATOM | 7693 | CA | LEU I | 78 | −0.064 | 8.098 | 29.659 | 1.00 | 17.98 | C | C |
| ATOM | 7694 | CB | LEU I | 78 | 1.419 | 7.775 | 29.528 | 1.00 | 17.72 | C | C |
| ATOM | 7695 | CG | LEU I | 78 | 2.275 | 9.033 | 29.591 | 1.00 | 17.51 | C | C |
| ATOM | 7696 | CD1 | LEU I | 78 | 3.746 | 8.676 | 29.494 | 1.00 | 17.41 | C | C |
| ATOM | 7697 | CD2 | LEU I | 78 | 1.859 | 9.984 | 28.480 | 1.00 | 17.62 | C | C |
| ATOM | 7698 | C | LEU I | 78 | −0.882 | 6.820 | 29.656 | 1.00 | 17.77 | C | C |
| ATOM | 7699 | O | LEU I | 78 | −1.665 | 6.576 | 28.739 | 1.00 | 17.89 | C | O |
| ATOM | 7700 | N | ILE I | 79 | −0.749 | 6.036 | 30.717 | 1.00 | 17.29 | C | N |
| ATOM | 7701 | CA | ILE I | 79 | −1.319 | 4.703 | 30.726 | 1.00 | 17.00 | C | C |
| ATOM | 7702 | CB | ILE I | 79 | −0.968 | 3.948 | 32.005 | 1.00 | 16.96 | C | C |
| ATOM | 7703 | CG1 | ILE I | 79 | 0.550 | 3.801 | 32.113 | 1.00 | 17.21 | C | C |
| ATOM | 7704 | CD1 | ILE I | 79 | 1.020 | 3.244 | 33.429 | 1.00 | 17.51 | C | C |
| ATOM | 7705 | CG2 | ILE I | 79 | −1.643 | 2.587 | 32.005 | 1.00 | 17.30 | C | C |
| ATOM | 7706 | C | ILE I | 79 | −2.828 | 4.750 | 30.547 | 1.00 | 16.83 | C | C |
| ATOM | 7707 | O | ILE I | 79 | −3.365 | 4.160 | 29.609 | 1.00 | 17.02 | C | O |
| ATOM | 7708 | N | LYS I | 80 | −3.616 | 5.565 | 31.220 | 1.00 | 16.55 | C | N |
| ATOM | 7709 | CA | LYS I | 80 | −5.043 | 5.447 | 30.835 | 1.00 | 16.38 | C | C |
| ATOM | 7710 | CB | LYS I | 80 | −5.918 | 6.332 | 31.725 | 1.00 | 16.73 | C | C |
| ATOM | 7711 | CG | LYS I | 80 | −7.025 | 7.055 | 30.971 | 1.00 | 17.99 | C | C |
| ATOM | 7712 | CD | LYS I | 80 | −8.237 | 7.295 | 31.854 | 1.00 | 20.71 | C | C |
| ATOM | 7713 | CE | LYS I | 80 | −9.171 | 6.094 | 31.833 | 1.00 | 22.92 | C | C |
| ATOM | 7714 | NZ | LYS I | 80 | −8.439 | 4.818 | 32.068 | 1.00 | 24.75 | C | N |
| ATOM | 7715 | C | LYS I | 80 | −5.386 | 5.716 | 29.328 | 1.00 | 15.79 | C | C |
| ATOM | 7716 | O | LYS I | 80 | −6.140 | 4.942 | 28.677 | 1.00 | 15.32 | C | O |
| ATOM | 7717 | N | GLN I | 81 | −4.802 | 6.770 | 28.763 | 1.00 | 15.48 | C | N |
| ATOM | 7718 | CA | GLN I | 81 | −5.092 | 7.178 | 27.383 | 1.00 | 15.33 | C | C |
| ATOM | 7719 | CB | GLN I | 81 | −4.378 | 8.495 | 27.068 | 1.00 | 15.70 | C | C |
| ATOM | 7720 | CG | GLN I | 81 | −4.522 | 9.557 | 28.146 | 1.00 | 17.88 | C | C |
| ATOM | 7721 | CD | GLN I | 81 | −3.631 | 10.759 | 27.903 | 1.00 | 20.46 | C | C |
| ATOM | 7722 | OE1 | GLN I | 81 | −3.065 | 10.916 | 26.822 | 1.00 | 21.59 | C | O |
| ATOM | 7723 | NE2 | GLN I | 81 | −3.498 | 11.613 | 28.913 | 1.00 | 20.26 | C | N |
| ATOM | 7724 | C | GLN I | 81 | −4.708 | 6.125 | 26.341 | 1.00 | 14.63 | C | C |
| ATOM | 7725 | O | GLN I | 81 | −5.438 | 5.881 | 25.360 | 1.00 | 14.60 | C | O |
| ATOM | 7726 | N | GLU I | 82 | −3.556 | 5.500 | 26.560 | 1.00 | 13.81 | C | N |
| ATOM | 7727 | CA | GLU I | 82 | −3.078 | 4.470 | 25.657 | 1.00 | 13.37 | C | C |
| ATOM | 7728 | CB | GLU I | 82 | −1.705 | 3.958 | 26.094 | 1.00 | 13.20 | C | C |
| ATOM | 7729 | CG | GLU I | 82 | −0.528 | 4.666 | 25.439 | 1.00 | 15.04 | C | C |
| ATOM | 7730 | CD | GLU I | 82 | −0.472 | 4.436 | 23.942 | 1.00 | 17.51 | C | C |
| ATOM | 7731 | OE1 | GLU I | 82 | 0.319 | 3.576 | 23.502 | 1.00 | 18.22 | C | O |
| ATOM | 7732 | OE2 | GLU I | 82 | −1.211 | 5.121 | 23.205 | 1.00 | 17.87 | C | O |
| ATOM | 7733 | C | GLU I | 82 | −4.079 | 3.333 | 25.659 | 1.00 | 12.99 | C | C |
| ATOM | 7734 | O | GLU I | 82 | −4.400 | 2.779 | 24.610 | 1.00 | 12.89 | C | O |
| ATOM | 7735 | N | LEU I | 83 | −4.593 | 3.001 | 26.838 | 1.00 | 12.89 | C | N |
| ATOM | 7736 | CA | LEU I | 83 | −5.558 | 1.919 | 26.944 | 1.00 | 13.08 | C | C |
| ATOM | 7737 | CB | LEU I | 83 | −5.923 | 1.668 | 28.407 | 1.00 | 13.26 | C | C |
| ATOM | 7738 | CG | LEU I | 83 | −4.774 | 1.250 | 29.325 | 1.00 | 14.18 | C | C |
| ATOM | 7739 | CD1 | LEU I | 83 | −5.293 | 0.948 | 30.721 | 1.00 | 15.11 | C | C |
| ATOM | 7740 | CD2 | LEU I | 83 | −4.036 | 0.051 | 28.748 | 1.00 | 14.79 | C | C |
| ATOM | 7741 | C | LEU I | 83 | −6.808 | 2.252 | 26.144 | 1.00 | 13.18 | C | C |
| ATOM | 7742 | O | LEU I | 83 | −7.310 | 1.410 | 25.386 | 1.00 | 13.05 | C | O |
| ATOM | 7743 | N | ASP I | 84 | −7.286 | 3.490 | 26.251 | 1.00 | 13.56 | C | N |
| ATOM | 7744 | CA | ASP I | 84 | −8.478 | 3.834 | 25.467 | 1.00 | 14.15 | C | C |
| ATOM | 7745 | CB | ASP I | 84 | −8.946 | 5.258 | 25.781 | 1.00 | 14.80 | C | C |
| ATOM | 7746 | CG | ASP I | 84 | −9.044 | 5.529 | 27.268 | 1.00 | 18.26 | C | C |
| ATOM | 7747 | OD1 | ASP I | 84 | −10.133 | 5.330 | 27.846 | 1.00 | 21.11 | C | O |
| ATOM | 7748 | OD2 | ASP I | 84 | −8.032 | 5.959 | 27.858 | 1.00 | 22.12 | C | O |
| ATOM | 7749 | C | ASP I | 84 | −8.226 | 3.703 | 23.954 | 1.00 | 13.56 | C | C |
| ATOM | 7750 | O | ASP I | 84 | −9.062 | 3.149 | 23.192 | 1.00 | 13.70 | C | O |
| ATOM | 7751 | N | LYS I | 85 | −7.062 | 4.182 | 23.517 | 1.00 | 12.87 | C | N |
| ATOM | 7752 | CA | LYS I | 85 | −6.752 | 4.121 | 22.089 | 1.00 | 12.31 | C | C |
| ATOM | 7753 | CB | LYS I | 85 | −5.424 | 4.816 | 21.793 | 1.00 | 12.41 | C | C |
| ATOM | 7754 | CG | LYS I | 85 | −5.118 | 4.954 | 20.308 | 1.00 | 12.26 | C | C |
| ATOM | 7755 | CD | LYS I | 85 | −3.659 | 5.305 | 20.076 | 1.00 | 12.72 | C | C |
| ATOM | 7756 | CE | LYS I | 85 | −2.751 | 4.171 | 20.519 | 1.00 | 14.40 | C | C |
| ATOM | 7757 | NZ | LYS I | 85 | −1.316 | 4.445 | 20.230 | 1.00 | 16.45 | C | N |
| ATOM | 7758 | C | LYS I | 85 | −6.691 | 2.671 | 21.621 | 1.00 | 11.85 | C | C |
| ATOM | 7759 | O | LYS I | 85 | −7.187 | 2.312 | 20.537 | 1.00 | 11.66 | C | O |
| ATOM | 7760 | N | TYR I | 86 | −6.085 | 1.836 | 22.457 | 1.00 | 11.32 | C | N |
| ATOM | 7761 | CA | TYR I | 86 | −5.931 | 0.432 | 22.135 | 1.00 | 11.07 | C | C |
| ATOM | 7762 | CB | TYR I | 86 | −5.115 | −0.290 | 23.203 | 1.00 | 11.12 | C | C |
| ATOM | 7763 | CG | TYR I | 86 | −5.391 | −1.773 | 23.243 | 1.00 | 11.30 | C | C |
| ATOM | 7764 | CD1 | TYR I | 86 | −4.884 | −2.621 | 22.268 | 1.00 | 12.16 | C | C |
| ATOM | 7765 | CE1 | TYR I | 86 | −5.140 | −3.977 | 22.297 | 1.00 | 12.39 | C | C |
| ATOM | 7766 | CZ | TYR I | 86 | −5.916 | −4.500 | 23.308 | 1.00 | 12.62 | C | C |
| ATOM | 7767 | OH | TYR I | 86 | −6.174 | −5.852 | 23.343 | 1.00 | 12.72 | C | O |
| ATOM | 7768 | CE2 | TYR I | 86 | −6.434 | −3.680 | 24.286 | 1.00 | 11.38 | C | C |
| ATOM | 7769 | CD2 | TYR I | 86 | −6.173 | −2.325 | 24.248 | 1.00 | 11.05 | C | C |
| ATOM | 7770 | C | TYR I | 86 | −7.290 | −0.219 | 22.008 | 1.00 | 10.75 | C | C |
| ATOM | 7771 | O | TYR I | 86 | −7.517 | −1.004 | 21.098 | 1.00 | 10.45 | C | O |
| ATOM | 7772 | N | LYS I | 87 | −8.204 | 0.123 | 22.908 | 1.00 | 10.66 | C | N |
| ATOM | 7773 | CA | LYS I | 87 | −9.533 | −0.467 | 22.847 | 1.00 | 11.03 | C | C |
| ATOM | 7774 | CB | LYS I | 87 | −10.389 | 0.010 | 24.021 | 1.00 | 11.36 | C | C |
| ATOM | 7775 | CG | LYS I | 87 | −9.786 | −0.270 | 25.384 | 1.00 | 12.79 | C | C |
| ATOM | 7776 | CD | LYS I | 87 | −10.675 | 0.264 | 26.495 | 1.00 | 14.76 | C | C |
| ATOM | 7777 | CE | LYS I | 87 | −10.068 | −0.017 | 27.859 | 1.00 | 15.29 | C | C |
| ATOM | 7778 | NZ | LYS I | 87 | −10.924 | 0.485 | 28.970 | 1.00 | 16.27 | C | N |
| ATOM | 7779 | C | LYS I | 87 | −10.208 | −0.098 | 21.530 | 1.00 | 10.83 | C | C |
| ATOM | 7780 | O | LYS I | 87 | −10.800 | −0.963 | 20.857 | 1.00 | 11.11 | C | O |
| ATOM | 7781 | N | ASN I | 88 | −10.089 | 1.168 | 21.126 | 1.00 | 10.52 | C | N |
| ATOM | 7782 | CA | ASN I | 88 | −10.721 | 1.545 | 19.858 | 1.00 | 10.21 | C | C |
| ATOM | 7783 | CB | ASN I | 88 | −10.592 | 3.050 | 19.614 | 1.00 | 10.61 | C | C |
| ATOM | 7784 | CG | ASN I | 88 | −11.178 | 3.480 | 18.281 | 1.00 | 11.81 | C | C |
| ATOM | 7785 | OD1 | ASN I | 88 | −12.114 | 2.864 | 17.772 | 1.00 | 12.97 | C | O |
| ATOM | 7786 | ND2 | ASN I | 88 | −10.625 | 4.543 | 17.709 | 1.00 | 13.82 | C | N |
| ATOM | 7787 | C | ASN I | 88 | −10.128 | 0.767 | 18.674 | 1.00 | 9.42 | C | C |
| ATOM | 7788 | O | ASN I | 88 | −10.858 | 0.237 | 17.796 | 1.00 | 8.94 | C | O |
| ATOM | 7789 | N | ALA I | 89 | −8.802 | 0.652 | 18.677 | 1.00 | 8.77 | C | N |
| ATOM | 7790 | CA | ALA I | 89 | −8.130 | −0.041 | 17.586 | 1.00 | 8.21 | C | C |
| ATOM | 7791 | CB | ALA I | 89 | −6.623 | 0.040 | 17.751 | 1.00 | 7.99 | C | C |
| ATOM | 7792 | C | ALA I | 89 | −8.588 | −1.493 | 17.543 | 1.00 | 7.78 | C | C |
| ATOM | 7793 | O | ALA I | 89 | −8.826 | −2.057 | 16.472 | 1.00 | 7.88 | C | O |
| ATOM | 7794 | N | VAL I | 90 | −8.718 | −2.086 | 18.724 | 1.00 | 7.01 | C | N |
| ATOM | 7795 | CA | VAL I | 90 | −9.124 | −3.474 | 18.849 | 1.00 | 6.26 | C | C |
| ATOM | 7796 | CB | VAL I | 90 | −9.079 | −3.943 | 20.313 | 1.00 | 5.91 | C | C |
| ATOM | 7797 | CG1 | VAL I | 90 | −9.557 | −5.381 | 20.422 | 1.00 | 5.92 | C | C |
| ATOM | 7798 | CG2 | VAL I | 90 | −7.671 | −3.802 | 20.871 | 1.00 | 5.26 | C | C |
| ATOM | 7799 | C | VAL I | 90 | −10.524 | −3.689 | 18.299 | 1.00 | 6.25 | C | C |
| ATOM | 7800 | O | VAL I | 90 | −10.765 | −4.663 | 17.592 | 1.00 | 6.32 | C | O |
| ATOM | 7801 | N | THR I | 91 | −11.445 | −2.776 | 18.592 | 1.00 | 6.24 | C | N |
| ATOM | 7802 | CA | THR I | 91 | −12.799 | −2.932 | 18.065 | 1.00 | 6.68 | C | C |
| ATOM | 7803 | CB | THR I | 91 | −13.734 | −1.829 | 18.584 | 1.00 | 6.62 | C | C |
| ATOM | 7804 | OG1 | THR I | 91 | −13.711 | −1.819 | 20.017 | 1.00 | 7.19 | C | O |
| ATOM | 7805 | CG2 | THR I | 91 | −15.157 | −2.071 | 18.106 | 1.00 | 7.27 | C | C |
| ATOM | 7806 | C | THR I | 91 | −12.768 | −2.885 | 16.536 | 1.00 | 7.02 | C | C |
| ATOM | 7807 | O | THR I | 91 | −13.430 | −3.703 | 15.833 | 1.00 | 7.16 | C | O |
| ATOM | 7808 | N | GLU I | 92 | −11.970 | −1.955 | 16.011 | 1.00 | 7.31 | C | N |
| ATOM | 7809 | CA | GLU I | 92 | −11.889 | −1.863 | 14.558 | 1.00 | 7.81 | C | C |
| ATOM | 7810 | CB | GLU I | 92 | −11.017 | −0.680 | 14.131 | 1.00 | 8.23 | C | C |
| ATOM | 7811 | CG | GLU I | 92 | −11.774 | 0.630 | 13.973 | 1.00 | 10.91 | C | C |
| ATOM | 7812 | CD | GLU I | 92 | −12.907 | 0.529 | 12.969 | 1.00 | 14.95 | C | C |
| ATOM | 7813 | OE1 | GLU I | 92 | −12.739 | 1.010 | 11.830 | 1.00 | 16.14 | C | O |
| ATOM | 7814 | OE2 | GLU I | 92 | −13.968 | −0.027 | 13.320 | 1.00 | 16.53 | C | O |
| ATOM | 7815 | C | GLU I | 92 | −11.349 | −3.164 | 13.959 | 1.00 | 7.47 | C | C |
| ATOM | 7816 | O | GLU I | 92 | −11.911 | −3.691 | 12.994 | 1.00 | 7.32 | C | O |
| ATOM | 7817 | N | LEU I | 93 | −10.303 | −3.716 | 14.568 | 1.00 | 7.30 | C | N |
| ATOM | 7818 | CA | LEU I | 93 | −9.703 | −4.950 | 14.062 | 1.00 | 7.47 | C | C |
| ATOM | 7819 | CB | LEU I | 93 | −8.429 | −5.299 | 14.830 | 1.00 | 7.15 | C | C |
| ATOM | 7820 | CG | LEU I | 93 | −7.212 | −4.438 | 14.490 | 1.00 | 7.20 | C | C |
| ATOM | 7821 | CD1 | LEU I | 93 | −5.977 | −4.957 | 15.204 | 1.00 | 7.93 | C | C |
| ATOM | 7822 | CD2 | LEU I | 93 | −6.989 | −4.392 | 12.984 | 1.00 | 6.91 | C | C |
| ATOM | 7823 | C | LEU I | 93 | −10.705 | −6.097 | 14.123 | 1.00 | 7.89 | C | C |
| ATOM | 7824 | O | LEU I | 93 | −10.764 | −6.947 | 13.235 | 1.00 | 7.94 | C | O |
| ATOM | 7825 | N | GLN I | 94 | −11.486 | −6.094 | 15.193 | 1.00 | 8.31 | C | N |
| ATOM | 7826 | CA | GLN I | 94 | −12.514 | −7.088 | 15.461 | 1.00 | 9.05 | C | C |
| ATOM | 7827 | CB | GLN I | 94 | −13.131 | −6.862 | 16.845 | 1.00 | 9.18 | C | C |
| ATOM | 7828 | CG | GLN I | 94 | −12.158 | −7.052 | 17.996 | 1.00 | 10.79 | C | C |
| ATOM | 7829 | CD | GLN I | 94 | −12.831 | −6.966 | 19.353 | 1.00 | 13.82 | C | C |
| ATOM | 7830 | OE1 | GLN I | 94 | −14.042 | −6.762 | 19.448 | 1.00 | 14.98 | C | O |
| ATOM | 7831 | NE2 | GLN I | 94 | −12.047 | −7.125 | 20.412 | 1.00 | 15.25 | C | N |
| ATOM | 7832 | C | GLN I | 94 | −13.615 | −7.127 | 14.405 | 1.00 | 9.25 | C | C |
| ATOM | 7833 | O | GLN I | 94 | −14.122 | −8.204 | 14.093 | 1.00 | 9.43 | C | O |
| ATOM | 7834 | N | LEU I | 95 | −14.007 | −5.977 | 13.858 | 1.00 | 9.55 | C | N |
| ATOM | 7835 | CA | LEU I | 95 | −15.128 | −6.009 | 12.888 | 1.00 | 10.17 | C | C |
| ATOM | 7836 | CB | LEU I | 95 | −15.535 | −4.582 | 12.510 | 1.00 | 9.79 | C | C |
| ATOM | 7837 | CG | LEU I | 95 | −16.117 | −3.716 | 13.628 | 1.00 | 8.86 | C | C |
| ATOM | 7838 | CD1 | LEU I | 95 | −16.419 | −2.315 | 13.117 | 1.00 | 8.31 | C | C |
| ATOM | 7839 | CD2 | LEU I | 95 | −17.367 | −4.359 | 14.211 | 1.00 | 7.62 | C | C |
| ATOM | 7840 | C | LEU I | 95 | −14.958 | −6.865 | 11.591 | 1.00 | 11.22 | C | C |
| ATOM | 7841 | O | LEU I | 95 | −15.902 | −7.505 | 11.125 | 1.00 | 11.55 | C | O |
| ATOM | 7842 | N | LEU I | 96 | −13.751 | −6.843 | 11.039 | 1.00 | 12.40 | C | N |
| ATOM | 7843 | CA | LEU I | 96 | −13.313 | −7.374 | 9.755 | 1.00 | 13.66 | C | C |
| ATOM | 7844 | CB | LEU I | 96 | −11.796 | −7.246 | 9.601 | 1.00 | 13.38 | C | C |
| ATOM | 7845 | CG | LEU I | 96 | −11.288 | −5.847 | 9.244 | 1.00 | 13.32 | C | C |
| ATOM | 7846 | CD1 | LEU I | 96 | −9.901 | −5.910 | 8.624 | 1.00 | 12.84 | C | C |
| ATOM | 7847 | CD2 | LEU I | 96 | −12.256 | −5.161 | 8.301 | 1.00 | 13.38 | C | C |
| ATOM | 7848 | C | LEU I | 96 | −13.765 | −8.817 | 9.534 | 1.00 | 14.97 | C | C |
| ATOM | 7849 | O | LEU I | 96 | −13.908 | −9.257 | 8.395 | 1.00 | 15.21 | C | O |
| ATOM | 7850 | N | MET I | 97 | −14.125 | −9.501 | 10.615 | 1.00 | 16.74 | C | N |
| ATOM | 7851 | CA | MET I | 97 | −14.122 | −10.962 | 10.625 | 1.00 | 18.96 | C | C |
| ATOM | 7852 | CB | MET I | 97 | −13.987 | −11.498 | 12.053 | 1.00 | 19.12 | C | C |
| ATOM | 7853 | CG | MET I | 97 | −12.742 | −11.021 | 12.784 | 1.00 | 21.87 | C | C |
| ATOM | 7854 | SD | MET I | 97 | −11.220 | −11.692 | 12.090 | 1.00 | 27.46 | C | S |
| ATOM | 7855 | CE | MET I | 97 | −11.585 | −11.630 | 10.338 | 1.00 | 24.25 | C | C |
| ATOM | 7856 | C | MET I | 97 | −15.339 | −11.576 | 9.929 | 1.00 | 20.09 | C | C |
| ATOM | 7857 | O | MET I | 97 | −15.246 | −12.655 | 9.343 | 1.00 | 20.58 | C | O |
| ATOM | 7858 | N | GLN I | 98 | −16.462 | −10.865 | 9.950 | 1.00 | 21.33 | C | N |
| ATOM | 7859 | CA | GLN I | 98 | −17.706 | −11.392 | 9.401 | 1.00 | 22.21 | C | C |
| ATOM | 7860 | CB | GLN I | 98 | −18.708 | −11.678 | 10.521 | 1.00 | 22.53 | C | C |
| ATOM | 7861 | CG | GLN I | 98 | −18.099 | −12.341 | 11.742 | 1.00 | 23.85 | C | C |
| ATOM | 7862 | CD | GLN I | 98 | −17.673 | −11.337 | 12.792 | 1.00 | 25.21 | C | C |
| ATOM | 7863 | OE1 | GLN I | 98 | −16.838 | −10.469 | 12.535 | 1.00 | 24.47 | C | O |
| ATOM | 7864 | NE2 | GLN I | 98 | −18.199 | −11.489 | 14.002 | 1.00 | 24.85 | C | N |
| ATOM | 7865 | C | GLN I | 98 | −18.318 | −10.439 | 8.378 | 1.00 | 22.15 | C | C |
| ATOM | 7866 | O | GLN I | 98 | −17.765 | −9.376 | 8.095 | 1.00 | 21.77 | C | O |
| ATOM | 7867 | N | PHE I | 137 | −22.709 | −16.921 | 4.271 | 1.00 | 33.25 | C | N |
| ATOM | 7868 | CA | PHE I | 137 | −21.644 | −16.142 | 3.651 | 1.00 | 36.79 | C | C |
| ATOM | 7869 | CB | PHE I | 137 | −21.061 | −16.892 | 2.453 | 1.00 | 20.00 | C | C |
| ATOM | 7870 | CG | PHE I | 137 | −20.354 | −18.166 | 2.825 | 1.00 | 20.00 | C | C |
| ATOM | 7871 | CD1 | PHE I | 137 | −19.059 | −18.134 | 3.318 | 1.00 | 20.00 | C | C |
| ATOM | 7872 | CE1 | PHE I | 137 | −18.416 | −19.301 | 3.700 | 1.00 | 20.00 | C | C |
| ATOM | 7873 | CZ | PHE I | 137 | −19.092 | −20.504 | 3.660 | 1.00 | 20.00 | C | C |
| ATOM | 7874 | CE2 | PHE I | 137 | −20.414 | −20.534 | 3.260 | 1.00 | 20.00 | C | C |
| ATOM | 7875 | CD2 | PHE I | 137 | −21.046 | −19.365 | 2.871 | 1.00 | 20.00 | C | C |
| ATOM | 7876 | C | PHE I | 137 | −22.138 | −14.765 | 3.226 | 1.00 | 48.73 | C | C |
| ATOM | 7877 | O | PHE I | 137 | −21.342 | −13.888 | 2.890 | 1.00 | 49.28 | C | O |
| ATOM | 7878 | N | LEU I | 138 | −23.454 | −14.580 | 3.253 | 1.00 | 42.04 | C | N |
| ATOM | 7879 | CA | LEU I | 138 | −24.055 | −13.275 | 2.999 | 1.00 | 44.26 | C | C |
| ATOM | 7880 | CB | LEU I | 138 | −25.581 | −13.366 | 3.047 | 1.00 | 20.00 | C | C |
| ATOM | 7881 | CG | LEU I | 138 | −26.248 | −14.219 | 1.966 | 1.00 | 20.00 | C | C |
| ATOM | 7882 | CD1 | LEU I | 138 | −27.748 | −14.301 | 2.197 | 1.00 | 20.00 | C | C |
| ATOM | 7883 | CD2 | LEU I | 138 | −25.944 | −13.673 | 0.580 | 1.00 | 20.00 | C | C |
| ATOM | 7884 | C | LEU I | 138 | −23.565 | −12.242 | 4.006 | 1.00 | 43.68 | C | C |
| ATOM | 7885 | O | LEU I | 138 | −23.730 | −11.038 | 3.804 | 1.00 | 50.27 | C | O |
| ATOM | 7886 | N | GLY I | 139 | −22.983 | −12.724 | 5.100 | 1.00 | 38.56 | C | N |
| ATOM | 7887 | CA | GLY I | 139 | −22.415 | −11.853 | 6.126 | 1.00 | 47.99 | C | C |
| ATOM | 7888 | C | GLY I | 139 | −21.274 | −10.985 | 5.625 | 1.00 | 44.53 | C | C |
| ATOM | 7889 | O | GLY I | 139 | −20.971 | −9.947 | 6.213 | 1.00 | 32.21 | C | O |
| ATOM | 7890 | N | PHE I | 140 | −20.649 | −11.402 | 4.528 | 1.00 | 46.33 | C | N |
| ATOM | 7891 | CA | PHE I | 140 | −19.494 | −10.693 | 3.986 | 1.00 | 42.95 | C | C |
| ATOM | 7892 | CB | PHE I | 140 | −18.549 | −11.668 | 3.281 | 1.00 | 20.00 | C | C |
| ATOM | 7893 | CG | PHE I | 140 | −17.916 | −12.677 | 4.202 | 1.00 | 20.00 | C | C |
| ATOM | 7894 | CD1 | PHE I | 140 | −16.781 | −12.356 | 4.929 | 1.00 | 20.00 | C | C |
| ATOM | 7895 | CE1 | PHE I | 140 | −16.194 | −13.284 | 5.769 | 1.00 | 20.00 | C | C |
| ATOM | 7896 | CZ | PHE I | 140 | −16.731 | −14.551 | 5.879 | 1.00 | 20.00 | C | C |
| ATOM | 7897 | CE2 | PHE I | 140 | −17.854 | −14.888 | 5.150 | 1.00 | 20.00 | C | C |
| ATOM | 7898 | CD2 | PHE I | 140 | −18.434 | −13.958 | 4.308 | 1.00 | 20.00 | C | C |
| ATOM | 7899 | C | PHE I | 140 | −19.898 | −9.568 | 3.032 | 1.00 | 38.13 | C | C |
| ATOM | 7900 | O | PHE I | 140 | −19.052 | −8.795 | 2.581 | 1.00 | 57.33 | C | O |
| ATOM | 7901 | N | LEU I | 141 | −21.191 | −9.475 | 2.736 | 1.00 | 35.20 | C | N |
| ATOM | 7902 | CA | LEU I | 141 | −21.690 | −8.512 | 1.759 | 1.00 | 27.01 | C | C |
| ATOM | 7903 | CB | LEU I | 141 | −21.184 | −7.103 | 2.079 | 1.00 | 20.00 | C | C |
| ATOM | 7904 | CG | LEU I | 141 | −21.717 | −6.450 | 3.356 | 1.00 | 20.00 | C | C |
| ATOM | 7905 | CD1 | LEU I | 141 | −20.991 | −5.144 | 3.632 | 1.00 | 20.00 | C | C |
| ATOM | 7906 | CD2 | LEU I | 141 | −23.216 | −6.216 | 3.252 | 1.00 | 20.00 | C | C |
| ATOM | 7907 | C | LEU I | 141 | −21.292 | −8.918 | 0.341 | 1.00 | 35.79 | C | C |
| ATOM | 7908 | O | LEU I | 141 | −21.674 | −9.989 | −0.130 | 1.00 | 46.92 | C | O |
| ATOM | 7909 | N | LEU I | 142 | −20.469 | −8.099 | −0.306 | 1.00 | 38.67 | C | N |
| ATOM | 7910 | CA | LEU I | 142 | −19.832 | −8.492 | −1.559 | 1.00 | 33.84 | C | C |
| ATOM | 7911 | CB | LEU I | 142 | −20.017 | −7.408 | −2.618 | 1.00 | 20.00 | C | C |
| ATOM | 7912 | CG | LEU I | 142 | −21.465 | −7.163 | −3.040 | 1.00 | 20.00 | C | C |
| ATOM | 7913 | CD1 | LEU I | 142 | −21.584 | −5.883 | −3.848 | 1.00 | 20.00 | C | C |
| ATOM | 7914 | CD2 | LEU I | 142 | −21.999 | −8.350 | −3.827 | 1.00 | 20.00 | C | C |
| ATOM | 7915 | C | LEU I | 142 | −18.352 | −8.799 | −1.363 | 1.00 | 36.89 | C | C |
| ATOM | 7916 | O | LEU I | 142 | −17.604 | −7.979 | −0.831 | 1.00 | 45.42 | C | O |
| ATOM | 7917 | N | GLY I | 143 | −17.970 | −10.034 | −1.673 | 1.00 | 26.84 | C | N |
| ATOM | 7918 | CA | GLY I | 143 | −16.624 | −10.516 | −1.387 | 1.00 | 27.41 | C | C |
| ATOM | 7919 | C | GLY I | 143 | −15.977 | −11.154 | −2.599 | 1.00 | 29.08 | C | C |
| ATOM | 7920 | O | GLY I | 143 | −16.645 | −11.813 | −3.396 | 1.00 | 40.24 | C | O |
| ATOM | 7921 | N | VAL I | 144 | −14.672 | −10.957 | −2.742 | 1.00 | 30.17 | C | N |
| ATOM | 7922 | CA | VAL I | 144 | −13.928 | −11.605 | −3.814 | 1.00 | 34.15 | C | C |
| ATOM | 7923 | CB | VAL I | 144 | −12.559 | −10.921 | −4.058 | 1.00 | 20.00 | C | C |
| ATOM | 7924 | CG1 | VAL I | 144 | −11.826 | −11.591 | −5.212 | 1.00 | 20.00 | C | C |
| ATOM | 7925 | CG2 | VAL I | 144 | −12.750 | −9.434 | −4.339 | 1.00 | 20.00 | C | C |
| ATOM | 7926 | C | VAL I | 144 | −13.732 | −13.091 | −3.508 | 1.00 | 36.67 | C | C |
| ATOM | 7927 | O | VAL I | 144 | −14.071 | −13.558 | −2.419 | 1.00 | 42.49 | C | O |
| ATOM | 7928 | N | GLY I | 145 | −13.320 | −13.847 | −4.521 | 1.00 | 47.09 | C | N |
| ATOM | 7929 | CA | GLY I | 145 | −12.893 | −15.227 | −4.321 | 1.00 | 44.51 | C | C |
| ATOM | 7930 | C | GLY I | 145 | −11.950 | −15.377 | −3.141 | 1.00 | 44.94 | C | C |
| ATOM | 7931 | O | GLY I | 145 | −12.181 | −16.196 | −2.253 | 1.00 | 40.58 | C | O |
| ATOM | 7932 | N | SER I | 146 | −10.905 | −14.560 | −3.085 | 1.00 | 42.06 | C | N |
| ATOM | 7933 | CA | SER I | 146 | −9.931 | −14.673 | −2.000 | 1.00 | 47.39 | C | C |
| ATOM | 7934 | CB | SER I | 146 | −8.758 | −13.718 | −2.236 | 1.00 | 20.00 | C | C |
| ATOM | 7935 | OG | SER I | 146 | −9.200 | −12.373 | −2.297 | 1.00 | 20.00 | C | O |
| ATOM | 7936 | C | SER I | 146 | −10.535 | −14.425 | −0.613 | 1.00 | 51.48 | C | C |
| ATOM | 7937 | O | SER I | 146 | −10.211 | −15.129 | 0.351 | 1.00 | 47.45 | C | O |
| ATOM | 7938 | N | ALA I | 147 | −11.415 | −13.434 | −0.514 | 1.00 | 39.51 | C | N |
| ATOM | 7939 | CA | ALA I | 147 | −12.026 | −13.082 | 0.766 | 1.00 | 43.38 | C | C |
| ATOM | 7940 | CB | ALA I | 147 | −12.866 | −11.821 | 0.621 | 1.00 | 20.00 | C | C |
| ATOM | 7941 | C | ALA I | 147 | −12.865 | −14.218 | 1.348 | 1.00 | 47.39 | C | C |
| ATOM | 7942 | O | ALA I | 147 | −12.823 | −14.476 | 2.554 | 1.00 | 50.18 | C | O |
| ATOM | 7943 | N | ILE I | 148 | −13.624 | −14.894 | 0.492 | 1.00 | 44.67 | C | N |
| ATOM | 7944 | CA | ILE I | 148 | −14.453 | −16.007 | 0.937 | 1.00 | 43.56 | C | C |
| ATOM | 7945 | CB | ILE I | 148 | −15.322 | −16.558 | −0.208 | 1.00 | 20.00 | C | C |
| ATOM | 7946 | CG1 | ILE I | 148 | −16.249 | −15.466 | −0.746 | 1.00 | 20.00 | C | C |
| ATOM | 7947 | CD1 | ILE I | 148 | −17.141 | −15.925 | −1.878 | 1.00 | 20.00 | C | C |
| ATOM | 7948 | CG2 | ILE I | 148 | −16.127 | −17.759 | 0.265 | 1.00 | 20.00 | C | C |
| ATOM | 7949 | C | ILE I | 148 | −13.575 | −17.126 | 1.483 | 1.00 | 51.18 | C | C |
| ATOM | 7950 | O | ILE I | 148 | −13.886 | −17.736 | 2.510 | 1.00 | 39.17 | C | O |
| ATOM | 7951 | N | ALA I | 149 | −12.468 | −17.382 | 0.794 | 1.00 | 42.17 | C | N |
| ATOM | 7952 | CA | ALA I | 149 | −11.531 | −18.412 | 1.217 | 1.00 | 36.76 | C | C |
| ATOM | 7953 | CB | ALA I | 149 | −10.424 | −18.578 | 0.189 | 1.00 | 20.00 | C | C |
| ATOM | 7954 | C | ALA I | 149 | −10.949 | −18.044 | 2.574 | 1.00 | 45.19 | C | C |
| ATOM | 7955 | O | ALA I | 149 | −10.806 | −18.897 | 3.451 | 1.00 | 47.83 | C | O |
| ATOM | 7956 | N | SER I | 150 | −10.632 | −16.765 | 2.748 | 1.00 | 43.64 | C | N |
| ATOM | 7957 | CA | SER I | 150 | −10.084 | −16.296 | 4.014 | 1.00 | 43.46 | C | C |
| ATOM | 7958 | CB | SER I | 150 | −9.701 | −14.818 | 3.919 | 1.00 | 20.00 | C | C |
| ATOM | 7959 | OG | SER I | 150 | −10.828 | −14.018 | 3.605 | 1.00 | 20.00 | C | O |
| ATOM | 7960 | C | SER I | 150 | −11.093 | −16.507 | 5.138 | 1.00 | 44.80 | C | C |
| ATOM | 7961 | O | SER I | 150 | −10.733 | −16.932 | 6.238 | 1.00 | 41.55 | C | O |
| ATOM | 7962 | N | GLY I | 151 | −12.361 | −16.222 | 4.854 | 1.00 | 27.01 | C | N |
| ATOM | 7963 | CA | GLY I | 151 | −13.413 | −16.410 | 5.836 | 1.00 | 35.21 | C | C |
| ATOM | 7964 | C | GLY I | 151 | −13.553 | −17.871 | 6.219 | 1.00 | 42.40 | C | C |
| ATOM | 7965 | O | GLY I | 151 | −13.742 | −18.212 | 7.394 | 1.00 | 45.11 | C | O |
| ATOM | 7966 | N | VAL I | 152 | −13.454 | −18.742 | 5.219 | 1.00 | 40.20 | C | N |
| ATOM | 7967 | CA | VAL I | 152 | −13.548 | −20.175 | 5.458 | 1.00 | 46.73 | C | C |
| ATOM | 7968 | CB | VAL I | 152 | −13.505 | −20.973 | 4.144 | 1.00 | 20.00 | C | C |
| ATOM | 7969 | CG1 | VAL I | 152 | −13.606 | −22.464 | 4.426 | 1.00 | 20.00 | C | C |
| ATOM | 7970 | CG2 | VAL I | 152 | −14.621 | −20.523 | 3.214 | 1.00 | 20.00 | C | C |
| ATOM | 7971 | C | VAL I | 152 | −12.396 | −20.615 | 6.351 | 1.00 | 48.62 | C | C |
| ATOM | 7972 | O | VAL I | 152 | −12.579 | −21.411 | 7.273 | 1.00 | 49.32 | C | O |
| ATOM | 7973 | N | ALA I | 153 | −11.212 | −20.074 | 6.083 | 1.00 | 35.09 | C | N |
| ATOM | 7974 | CA | ALA I | 153 | −10.030 | −20.403 | 6.867 | 1.00 | 42.37 | C | C |
| ATOM | 7975 | CB | ALA I | 153 | −8.798 | −19.741 | 6.274 | 1.00 | 20.00 | C | C |
| ATOM | 7976 | C | ALA I | 153 | −10.231 | −19.965 | 8.310 | 1.00 | 37.79 | C | C |
| ATOM | 7977 | O | ALA I | 153 | −9.860 | −20.676 | 9.244 | 1.00 | 39.25 | C | O |
| ATOM | 7978 | N | VAL I | 154 | −10.825 | −18.789 | 8.486 | 1.00 | 38.88 | C | N |
| ATOM | 7979 | CA | VAL I | 154 | −11.100 | −18.270 | 9.819 | 1.00 | 35.53 | C | C |
| ATOM | 7980 | CB | VAL I | 154 | −11.680 | −16.845 | 9.762 | 1.00 | 20.00 | C | C |
| ATOM | 7981 | CG1 | VAL I | 154 | −11.955 | −16.328 | 11.165 | 1.00 | 20.00 | C | C |
| ATOM | 7982 | CG2 | VAL I | 154 | −10.731 | −15.916 | 9.021 | 1.00 | 20.00 | C | C |
| ATOM | 7983 | C | VAL I | 154 | −12.071 | −19.175 | 10.572 | 1.00 | 41.00 | C | C |
| ATOM | 7984 | O | VAL I | 154 | −11.884 | −19.441 | 11.759 | 1.00 | 50.08 | C | O |
| ATOM | 7985 | N | SER I | 155 | −13.105 | −19.652 | 9.882 | 1.00 | 37.82 | C | N |
| ATOM | 7986 | CA | SER I | 155 | −14.071 | −20.556 | 10.508 | 1.00 | 44.68 | C | C |
| ATOM | 7987 | CB | SER I | 155 | −15.239 | −20.834 | 9.561 | 1.00 | 20.00 | C | C |
| ATOM | 7988 | OG | SER I | 155 | −14.790 | −21.436 | 8.360 | 1.00 | 20.00 | C | O |
| ATOM | 7989 | C | SER I | 155 | −13.400 | −21.867 | 10.917 | 1.00 | 41.82 | C | C |
| ATOM | 7990 | O | SER I | 155 | −13.667 | −22.425 | 11.982 | 1.00 | 49.37 | C | O |
| ATOM | 7991 | N | LYS I | 156 | −12.525 | −22.338 | 10.038 | 1.00 | 36.99 | C | N |
| ATOM | 7992 | CA | LYS I | 156 | −11.772 | −23.575 | 10.184 | 1.00 | 35.11 | C | C |
| ATOM | 7993 | CB | LYS I | 156 | −11.122 | −23.965 | 8.856 | 1.00 | 20.00 | C | C |
| ATOM | 7994 | CG | LYS I | 156 | −12.116 | −24.339 | 7.768 | 1.00 | 20.00 | C | C |
| ATOM | 7995 | CD | LYS I | 156 | −11.408 | −24.786 | 6.499 | 1.00 | 20.00 | C | C |
| ATOM | 7996 | CE | LYS I | 156 | −12.400 | −25.265 | 5.452 | 1.00 | 20.00 | C | C |
| ATOM | 7997 | NZ | LYS I | 156 | −11.733 | −25.581 | 4.158 | 1.00 | 20.00 | C | N |
| ATOM | 7998 | C | LYS I | 156 | −10.715 | −23.451 | 11.275 | 1.00 | 35.98 | C | C |
| ATOM | 7999 | O | LYS I | 156 | −10.361 | −24.436 | 11.922 | 1.00 | 42.31 | C | O |
| ATOM | 8000 | N | VAL I | 157 | −10.260 | −22.224 | 11.514 | 1.00 | 37.88 | C | N |
| ATOM | 8001 | CA | VAL I | 157 | −9.225 | −21.960 | 12.515 | 1.00 | 50.94 | C | C |
| ATOM | 8002 | CB | VAL I | 157 | −8.267 | −20.831 | 12.062 | 1.00 | 20.00 | C | C |
| ATOM | 8003 | CG1 | VAL I | 157 | −7.261 | −20.505 | 13.162 | 1.00 | 20.00 | C | C |
| ATOM | 8004 | CG2 | VAL I | 157 | −7.552 | −21.219 | 10.771 | 1.00 | 20.00 | C | C |
| ATOM | 8005 | C | VAL I | 157 | −9.832 | −21.596 | 13.871 | 1.00 | 47.82 | C | C |
| ATOM | 8006 | O | VAL I | 157 | −9.116 | −21.443 | 14.863 | 1.00 | 56.87 | C | O |
| ATOM | 8007 | N | LEU I | 158 | −11.156 | −21.533 | 13.947 | 1.00 | 39.52 | C | N |
| ATOM | 8008 | CA | LEU I | 158 | −11.838 | −20.969 | 15.112 | 1.00 | 50.27 | C | C |
| ATOM | 8009 | CB | LEU I | 158 | −13.351 | −20.935 | 14.869 | 1.00 | 20.00 | C | C |
| ATOM | 8010 | CG | LEU I | 158 | −13.827 | −20.109 | 13.673 | 1.00 | 20.00 | C | C |
| ATOM | 8011 | CD1 | LEU I | 158 | −15.343 | −20.163 | 13.552 | 1.00 | 20.00 | C | C |
| ATOM | 8012 | CD2 | LEU I | 158 | −13.346 | −18.671 | 13.788 | 1.00 | 20.00 | C | C |
| ATOM | 8013 | C | LEU I | 158 | −11.550 | −21.631 | 16.465 | 1.00 | 54.47 | C | C |
| ATOM | 8014 | O | LEU I | 158 | −11.417 | −20.934 | 17.477 | 1.00 | 49.26 | C | O |
| ATOM | 8015 | N | HIS I | 159 | −11.447 | −22.954 | 16.501 | 1.00 | 59.45 | C | N |
| ATOM | 8016 | CA | HIS I | 159 | −11.205 | −23.636 | 17.768 | 1.00 | 49.11 | C | C |
| ATOM | 8017 | CB | HIS I | 159 | −11.225 | −25.153 | 17.573 | 1.00 | 20.00 | C | C |
| ATOM | 8018 | CG | HIS I | 159 | −12.522 | −25.676 | 17.038 | 1.00 | 20.00 | C | C |
| ATOM | 8019 | ND1 | HIS I | 159 | −13.559 | −26.070 | 17.855 | 1.00 | 20.00 | C | N |
| ATOM | 8020 | CE1 | HIS I | 159 | −14.570 | −26.484 | 17.112 | 1.00 | 20.00 | C | C |
| ATOM | 8021 | NE2 | HIS I | 159 | −14.225 | −26.373 | 15.842 | 1.00 | 20.00 | C | N |
| ATOM | 8022 | CD2 | HIS I | 159 | −12.949 | −25.871 | 15.768 | 1.00 | 20.00 | C | C |
| ATOM | 8023 | C | HIS I | 159 | −9.874 | −23.203 | 18.385 | 1.00 | 44.09 | C | C |
| ATOM | 8024 | O | HIS I | 159 | −9.780 | −22.977 | 19.596 | 1.00 | 42.13 | C | O |
| ATOM | 8025 | N | LEU I | 160 | −8.851 | −23.072 | 17.546 | 1.00 | 37.55 | C | N |
| ATOM | 8026 | CA | LEU I | 160 | −7.536 | −22.649 | 18.011 | 1.00 | 43.52 | C | C |
| ATOM | 8027 | CB | LEU I | 160 | −6.520 | −22.704 | 16.869 | 1.00 | 20.00 | C | C |
| ATOM | 8028 | CG | LEU I | 160 | −6.299 | −24.073 | 16.224 | 1.00 | 20.00 | C | C |
| ATOM | 8029 | CD1 | LEU I | 160 | −5.268 | −23.982 | 15.109 | 1.00 | 20.00 | C | C |
| ATOM | 8030 | CD2 | LEU I | 160 | −5.877 | −25.097 | 17.266 | 1.00 | 20.00 | C | C |
| ATOM | 8031 | C | LEU I | 160 | −7.599 | −21.240 | 18.591 | 1.00 | 47.22 | C | C |
| ATOM | 8032 | O | LEU I | 160 | −6.984 | −20.951 | 19.619 | 1.00 | 43.25 | C | O |
| ATOM | 8033 | N | GLU I | 161 | −8.351 | −20.368 | 17.927 | 1.00 | 40.88 | C | N |
| ATOM | 8034 | CA | GLU I | 161 | −8.512 | −18.996 | 18.388 | 1.00 | 40.61 | C | C |
| ATOM | 8035 | CB | GLU I | 161 | −9.325 | −18.182 | 17.381 | 1.00 | 20.00 | C | C |
| ATOM | 8036 | CG | GLU I | 161 | −8.702 | −18.104 | 15.997 | 1.00 | 20.00 | C | C |
| ATOM | 8037 | CD | GLU I | 161 | −9.537 | −17.292 | 15.027 | 1.00 | 20.00 | C | C |
| ATOM | 8038 | OE1 | GLU I | 161 | −9.126 | −17.158 | 13.855 | 1.00 | 20.00 | C | O |
| ATOM | 8039 | OE2 | GLU I | 161 | −10.604 | −16.788 | 15.436 | 1.00 | 20.00 | C | O |
| ATOM | 8040 | C | GLU I | 161 | −9.195 | −18.983 | 19.749 | 1.00 | 39.76 | C | C |
| ATOM | 8041 | O | GLU I | 161 | −8.816 | −18.221 | 20.642 | 1.00 | 44.02 | C | O |
| ATOM | 8042 | N | GLY I | 162 | −10.199 | −19.840 | 19.906 | 1.00 | 34.18 | C | N |
| ATOM | 8043 | CA | GLY I | 162 | −10.907 | −19.934 | 21.169 | 1.00 | 35.01 | C | C |
| ATOM | 8044 | C | GLY I | 162 | −9.967 | −20.387 | 22.269 | 1.00 | 34.86 | C | C |
| ATOM | 8045 | O | GLY I | 162 | −9.997 | −19.864 | 23.386 | 1.00 | 47.81 | C | O |
| ATOM | 8046 | N | GLU I | 163 | −9.112 | −21.352 | 21.945 | 1.00 | 40.78 | C | N |
| ATOM | 8047 | CA | GLU I | 163 | −8.143 | −21.856 | 22.911 | 1.00 | 37.20 | C | C |
| ATOM | 8048 | CB | GLU I | 163 | −7.365 | −23.036 | 22.326 | 1.00 | 20.00 | C | C |
| ATOM | 8049 | CG | GLU I | 163 | −8.235 | −24.218 | 21.931 | 1.00 | 20.00 | C | C |
| ATOM | 8050 | CD | GLU I | 163 | −7.430 | −25.368 | 21.357 | 1.00 | 20.00 | C | C |
| ATOM | 8051 | OE1 | GLU I | 163 | −8.037 | −26.397 | 20.995 | 1.00 | 20.00 | C | O |
| ATOM | 8052 | OE2 | GLU I | 163 | −6.191 | −25.241 | 21.268 | 1.00 | 20.00 | C | O |
| ATOM | 8053 | C | GLU I | 163 | −7.183 | −20.745 | 23.324 | 1.00 | 46.12 | C | C |
| ATOM | 8054 | O | GLU I | 163 | −6.837 | −20.611 | 24.501 | 1.00 | 58.66 | C | O |
| ATOM | 8055 | N | VAL I | 164 | −6.761 | −19.944 | 22.350 | 1.00 | 42.23 | C | N |
| ATOM | 8056 | CA | VAL I | 164 | −5.857 | −18.834 | 22.620 | 1.00 | 35.45 | C | C |
| ATOM | 8057 | CB | VAL I | 164 | −5.429 | −18.124 | 21.323 | 1.00 | 20.00 | C | C |
| ATOM | 8058 | CG1 | VAL I | 164 | −4.487 | −16.971 | 21.635 | 1.00 | 20.00 | C | C |
| ATOM | 8059 | CG2 | VAL I | 164 | −4.775 | −19.112 | 20.369 | 1.00 | 20.00 | C | C |
| ATOM | 8060 | C | VAL I | 164 | −6.522 | −17.823 | 23.548 | 1.00 | 46.32 | C | C |
| ATOM | 8061 | O | VAL I | 164 | −5.892 | −17.301 | 24.472 | 1.00 | 45.08 | C | O |
| ATOM | 8062 | N | ASN I | 165 | −7.802 | −17.558 | 23.304 | 1.00 | 40.82 | C | N |
| ATOM | 8063 | CA | ASN I | 165 | −8.548 | −16.631 | 24.144 | 1.00 | 46.48 | C | C |
| ATOM | 8064 | CB | ASN I | 165 | −9.952 | −16.403 | 23.581 | 1.00 | 20.00 | C | C |
| ATOM | 8065 | CG | ASN I | 165 | −9.930 | −15.822 | 22.181 | 1.00 | 20.00 | C | C |
| ATOM | 8066 | OD1 | ASN I | 165 | −10.975 | −15.504 | 21.612 | 1.00 | 20.00 | C | O |
| ATOM | 8067 | ND2 | ASN I | 165 | −8.736 | −15.681 | 21.617 | 1.00 | 20.00 | C | N |
| ATOM | 8068 | C | ASN I | 165 | −8.631 | −17.160 | 25.569 | 1.00 | 50.90 | C | C |
| ATOM | 8069 | O | ASN I | 165 | −8.474 | −16.412 | 26.536 | 1.00 | 47.11 | C | O |
| ATOM | 8070 | N | LYS I | 166 | −8.857 | −18.464 | 25.688 | 1.00 | 48.36 | C | N |
| ATOM | 8071 | CA | LYS I | 166 | −8.945 | −19.120 | 26.986 | 1.00 | 38.71 | C | C |
| ATOM | 8072 | CB | LYS I | 166 | −9.329 | −20.592 | 26.818 | 1.00 | 20.00 | C | C |
| ATOM | 8073 | CG | LYS I | 166 | −10.649 | −20.811 | 26.097 | 1.00 | 20.00 | C | C |
| ATOM | 8074 | CD | LYS I | 166 | −10.952 | −22.292 | 25.936 | 1.00 | 20.00 | C | C |
| ATOM | 8075 | CE | LYS I | 166 | −12.264 | −22.511 | 25.200 | 1.00 | 20.00 | C | C |
| ATOM | 8076 | NZ | LYS I | 166 | −12.569 | −23.958 | 25.028 | 1.00 | 20.00 | C | N |
| ATOM | 8077 | C | LYS I | 166 | −7.623 | −19.006 | 27.738 | 1.00 | 45.07 | C | C |
| ATOM | 8078 | O | LYS I | 166 | −7.600 | −18.823 | 28.955 | 1.00 | 56.09 | C | O |
| ATOM | 8079 | N | ILE I | 167 | −6.525 | −19.121 | 26.999 | 1.00 | 42.02 | C | N |
| ATOM | 8080 | CA | ILE I | 167 | −5.184 | −19.080 | 27.576 | 1.00 | 43.86 | C | C |
| ATOM | 8081 | CB | ILE I | 167 | −4.207 | −19.975 | 26.789 | 1.00 | 20.00 | C | C |
| ATOM | 8082 | CG1 | ILE I | 167 | −4.685 | −21.429 | 26.804 | 1.00 | 20.00 | C | C |
| ATOM | 8083 | CD1 | ILE I | 167 | −3.778 | −22.377 | 26.050 | 1.00 | 20.00 | C | C |
| ATOM | 8084 | CG2 | ILE I | 167 | −2.803 | −19.865 | 27.362 | 1.00 | 20.00 | C | C |
| ATOM | 8085 | C | ILE I | 167 | −4.599 | −17.669 | 27.665 | 1.00 | 49.02 | C | C |
| ATOM | 8086 | O | ILE I | 167 | −3.448 | −17.497 | 28.066 | 1.00 | 37.71 | C | O |
| ATOM | 8087 | N | LYS I | 168 | −5.382 | −16.664 | 27.282 | 1.00 | 33.87 | C | N |
| ATOM | 8088 | CA | LYS I | 168 | −4.890 | −15.285 | 27.251 | 1.00 | 35.11 | C | C |
| ATOM | 8089 | CB | LYS I | 168 | −5.939 | −14.363 | 26.622 | 1.00 | 20.00 | C | C |
| ATOM | 8090 | CG | LYS I | 168 | −6.275 | −14.698 | 25.178 | 1.00 | 20.00 | C | C |
| ATOM | 8091 | CD | LYS I | 168 | −7.292 | −13.723 | 24.607 | 1.00 | 20.00 | C | C |
| ATOM | 8092 | CE | LYS I | 168 | −7.612 | −14.046 | 23.157 | 1.00 | 20.00 | C | C |
| ATOM | 8093 | NZ | LYS I | 168 | −8.599 | −13.092 | 22.580 | 1.00 | 20.00 | C | N |
| ATOM | 8094 | C | LYS I | 168 | −4.431 | −14.704 | 28.600 | 1.00 | 39.27 | C | C |
| ATOM | 8095 | O | LYS I | 168 | −3.414 | −14.012 | 28.655 | 1.00 | 32.39 | C | O |
| ATOM | 8096 | N | SER I | 169 | −5.164 | −14.973 | 29.677 | 1.00 | 41.30 | C | N |
| ATOM | 8097 | CA | SER I | 169 | −4.797 | −14.446 | 30.987 | 1.00 | 43.23 | C | C |
| ATOM | 8098 | CB | SER I | 169 | −5.756 | −14.957 | 32.064 | 1.00 | 20.00 | C | C |
| ATOM | 8099 | OG | SER I | 169 | −5.739 | −16.371 | 32.127 | 1.00 | 20.00 | C | O |
| ATOM | 8100 | C | SER I | 169 | −3.359 | −14.795 | 31.353 | 1.00 | 41.44 | C | C |
| ATOM | 8101 | O | SER I | 169 | −2.580 | −13.924 | 31.740 | 1.00 | 44.82 | C | O |
| ATOM | 8102 | N | ALA I | 170 | −3.013 | −16.072 | 31.235 | 1.00 | 39.78 | C | N |
| ATOM | 8103 | CA | ALA I | 170 | −1.644 | −16.517 | 31.469 | 1.00 | 41.01 | C | C |
| ATOM | 8104 | CB | ALA I | 170 | −1.541 | −18.025 | 31.320 | 1.00 | 20.00 | C | C |
| ATOM | 8105 | C | ALA I | 170 | −0.674 | −15.820 | 30.523 | 1.00 | 46.04 | C | C |
| ATOM | 8106 | O | ALA I | 170 | 0.396 | −15.373 | 30.935 | 1.00 | 46.67 | C | O |
| ATOM | 8107 | N | LEU I | 171 | −1.061 | −15.718 | 29.258 | 1.00 | 34.26 | C | N |
| ATOM | 8108 | CA | LEU I | 171 | −0.245 | −15.030 | 28.269 | 1.00 | 37.69 | C | C |
| ATOM | 8109 | CB | LEU I | 171 | −0.942 | −15.036 | 26.912 | 1.00 | 20.00 | C | C |
| ATOM | 8110 | CG | LEU I | 171 | −1.226 | −16.424 | 26.346 | 1.00 | 20.00 | C | C |
| ATOM | 8111 | CD1 | LEU I | 171 | −1.873 | −16.311 | 24.972 | 1.00 | 20.00 | C | C |
| ATOM | 8112 | CD2 | LEU I | 171 | 0.060 | −17.242 | 26.293 | 1.00 | 20.00 | C | C |
| ATOM | 8113 | C | LEU I | 171 | 0.053 | −13.598 | 28.702 | 1.00 | 44.67 | C | C |
| ATOM | 8114 | O | LEU I | 171 | 1.173 | −13.109 | 28.538 | 1.00 | 34.27 | C | O |
| ATOM | 8115 | N | LEU I | 172 | −0.935 | −12.955 | 29.314 | 1.00 | 34.80 | C | N |
| ATOM | 8116 | CA | LEU I | 172 | −0.817 | −11.555 | 29.692 | 1.00 | 28.87 | C | C |
| ATOM | 8117 | CB | LEU I | 172 | −2.199 | −10.971 | 29.993 | 1.00 | 20.00 | C | C |
| ATOM | 8118 | CG | LEU I | 172 | −3.198 | −10.968 | 28.835 | 1.00 | 20.00 | C | C |
| ATOM | 8119 | CD1 | LEU I | 172 | −4.534 | −10.393 | 29.283 | 1.00 | 20.00 | C | C |
| ATOM | 8120 | CD2 | LEU I | 172 | −2.643 | −10.187 | 27.668 | 1.00 | 20.00 | C | C |
| ATOM | 8121 | C | LEU I | 172 | 0.099 | −11.400 | 30.901 | 1.00 | 29.90 | C | C |
| ATOM | 8122 | O | LEU I | 172 | 0.896 | −10.462 | 30.972 | 1.00 | 41.09 | C | O |
| ATOM | 8123 | N | SER I | 173 | 0.016 | −12.355 | 31.824 | 1.00 | 35.08 | C | N |
| ATOM | 8124 | CA | SER I | 173 | 0.740 | −12.276 | 33.092 | 1.00 | 35.53 | C | C |
| ATOM | 8125 | CB | SER I | 173 | 0.110 | −13.206 | 34.132 | 1.00 | 20.00 | C | C |
| ATOM | 8126 | OG | SER I | 173 | 0.148 | −14.555 | 33.701 | 1.00 | 20.00 | C | O |
| ATOM | 8127 | C | SER I | 173 | 2.218 | −12.610 | 32.919 | 1.00 | 35.07 | C | C |
| ATOM | 8128 | O | SER I | 173 | 3.042 | −12.300 | 33.779 | 1.00 | 51.28 | C | O |
| ATOM | 8129 | N | THR I | 174 | 2.537 | −13.275 | 31.817 | 1.00 | 46.35 | C | N |
| ATOM | 8130 | CA | THR I | 174 | 3.894 | −13.733 | 31.561 | 1.00 | 43.83 | C | C |
| ATOM | 8131 | CB | THR I | 174 | 3.889 | −14.970 | 30.641 | 1.00 | 20.00 | C | C |
| ATOM | 8132 | OG1 | THR I | 174 | 3.327 | −14.620 | 29.371 | 1.00 | 20.00 | C | O |
| ATOM | 8133 | CG2 | THR I | 174 | 3.056 | −16.081 | 31.260 | 1.00 | 20.00 | C | C |
| ATOM | 8134 | C | THR I | 174 | 4.722 | −12.626 | 30.910 | 1.00 | 52.54 | C | C |
| ATOM | 8135 | O | THR I | 174 | 4.393 | −12.154 | 29.821 | 1.00 | 64.10 | C | O |
| ATOM | 8136 | N | ASN I | 175 | 5.771 | −12.188 | 31.599 | 1.00 | 55.57 | C | N |
| ATOM | 8137 | CA | ASN I | 175 | 6.735 | −11.267 | 31.008 | 1.00 | 40.58 | C | C |
| ATOM | 8138 | CB | ASN I | 175 | 7.507 | −10.521 | 32.099 | 1.00 | 20.00 | C | C |
| ATOM | 8139 | CG | ASN I | 175 | 6.646 | −9.518 | 32.845 | 1.00 | 20.00 | C | C |
| ATOM | 8140 | OD1 | ASN I | 175 | 5.640 | −9.033 | 32.324 | 1.00 | 20.00 | C | O |
| ATOM | 8141 | ND2 | ASN I | 175 | 7.043 | −9.196 | 34.072 | 1.00 | 20.00 | C | N |
| ATOM | 8142 | C | ASN I | 175 | 7.708 | −11.986 | 30.075 | 1.00 | 43.65 | C | C |
| ATOM | 8143 | O | ASN I | 175 | 8.525 | −12.793 | 30.521 | 1.00 | 53.11 | C | O |
| ATOM | 8144 | N | LYS I | 176 | 7.546 | −11.768 | 28.773 | 1.00 | 44.89 | C | N |
| ATOM | 8145 | CA | LYS I | 176 | 8.402 | −12.407 | 27.776 | 1.00 | 46.42 | C | C |
| ATOM | 8146 | CB | LYS I | 176 | 8.150 | −13.917 | 27.740 | 1.00 | 20.00 | C | C |
| ATOM | 8147 | CG | LYS I | 176 | 8.510 | −14.625 | 29.034 | 1.00 | 20.00 | C | C |
| ATOM | 8148 | CD | LYS I | 176 | 8.270 | −16.122 | 28.943 | 1.00 | 20.00 | C | C |
| ATOM | 8149 | CE | LYS I | 176 | 8.994 | −16.863 | 30.063 | 1.00 | 20.00 | C | C |
| ATOM | 8150 | NZ | LYS I | 176 | 9.447 | −18.223 | 29.648 | 1.00 | 20.00 | C | N |
| ATOM | 8151 | C | LYS I | 176 | 8.192 | −11.798 | 26.392 | 1.00 | 45.00 | C | C |
| ATOM | 8152 | O | LYS I | 176 | 7.084 | −11.379 | 26.053 | 1.00 | 60.64 | C | O |
| ATOM | 8153 | N | ALA I | 177 | 9.280 | −11.645 | 25.643 | 1.00 | 46.01 | C | N |
| ATOM | 8154 | CA | ALA I | 177 | 9.189 | −11.237 | 24.242 | 1.00 | 43.65 | C | C |
| ATOM | 8155 | CB | ALA I | 177 | 10.555 | −10.847 | 23.706 | 1.00 | 20.00 | C | C |
| ATOM | 8156 | C | ALA I | 177 | 8.592 | −12.349 | 23.395 | 1.00 | 41.42 | C | C |
| ATOM | 8157 | O | ALA I | 177 | 7.695 | −12.116 | 22.584 | 1.00 | 45.37 | C | O |
| ATOM | 8158 | N | VAL I | 178 | 9.140 | −13.549 | 23.545 | 1.00 | 35.70 | C | N |
| ATOM | 8159 | CA | VAL I | 178 | 8.580 | −14.725 | 22.902 | 1.00 | 43.93 | C | C |
| ATOM | 8160 | CB | VAL I | 178 | 9.495 | −15.243 | 21.780 | 1.00 | 20.00 | C | C |
| ATOM | 8161 | CG1 | VAL I | 178 | 8.898 | −16.490 | 21.143 | 1.00 | 20.00 | C | C |
| ATOM | 8162 | CG2 | VAL I | 178 | 9.714 | −14.159 | 20.734 | 1.00 | 20.00 | C | C |
| ATOM | 8163 | C | VAL I | 178 | 8.363 | −15.829 | 23.922 | 1.00 | 34.16 | C | C |
| ATOM | 8164 | O | VAL I | 178 | 9.115 | −15.950 | 24.889 | 1.00 | 44.45 | C | O |
| ATOM | 8165 | N | VAL I | 179 | 7.271 | −16.565 | 23.760 | 1.00 | 44.87 | C | N |
| ATOM | 8166 | CA | VAL I | 179 | 6.916 | −17.608 | 24.708 | 1.00 | 38.47 | C | C |
| ATOM | 8167 | CB | VAL I | 179 | 6.234 | −17.027 | 25.960 | 1.00 | 20.00 | C | C |
| ATOM | 8168 | CG1 | VAL I | 179 | 5.785 | −18.147 | 26.887 | 1.00 | 20.00 | C | C |
| ATOM | 8169 | CG2 | VAL I | 179 | 7.174 | −16.076 | 26.680 | 1.00 | 20.00 | C | C |
| ATOM | 8170 | C | VAL I | 179 | 6.003 | −18.648 | 24.074 | 1.00 | 44.79 | C | C |
| ATOM | 8171 | O | VAL I | 179 | 5.097 | −18.317 | 23.310 | 1.00 | 51.88 | C | O |
| ATOM | 8172 | N | SER I | 180 | 6.270 | −19.911 | 24.378 | 1.00 | 45.23 | C | N |
| ATOM | 8173 | CA | SER I | 180 | 5.428 | −21.002 | 23.914 | 1.00 | 48.99 | C | C |
| ATOM | 8174 | CB | SER I | 180 | 6.171 | −22.334 | 24.038 | 1.00 | 20.00 | C | C |
| ATOM | 8175 | OG | SER I | 180 | 6.627 | −22.542 | 25.364 | 1.00 | 20.00 | C | O |
| ATOM | 8176 | C | SER I | 180 | 4.118 | −21.053 | 24.698 | 1.00 | 48.15 | C | C |
| ATOM | 8177 | O | SER I | 180 | 4.116 | −21.309 | 25.901 | 1.00 | 60.42 | C | O |
| ATOM | 8178 | N | LEU I | 181 | 3.009 | −20.782 | 24.016 | 1.00 | 42.42 | C | N |
| ATOM | 8179 | CA | LEU I | 181 | 1.684 | −20.959 | 24.605 | 1.00 | 43.22 | C | C |
| ATOM | 8180 | CB | LEU I | 181 | 0.610 | −20.369 | 23.693 | 1.00 | 20.00 | C | C |
| ATOM | 8181 | CG | LEU I | 181 | 0.738 | −18.866 | 23.467 | 1.00 | 20.00 | C | C |
| ATOM | 8182 | CD1 | LEU I | 181 | −0.514 | −18.314 | 22.808 | 1.00 | 20.00 | C | C |
| ATOM | 8183 | CD2 | LEU I | 181 | 1.008 | −18.184 | 24.795 | 1.00 | 20.00 | C | C |
| ATOM | 8184 | C | LEU I | 181 | 1.388 | −22.428 | 24.863 | 1.00 | 29.14 | C | C |
| ATOM | 8185 | O | LEU I | 181 | 1.144 | −23.194 | 23.931 | 1.00 | 43.42 | C | O |
| ATOM | 8186 | N | SER I | 182 | 1.361 | −22.807 | 26.136 | 1.00 | 41.83 | C | N |
| ATOM | 8187 | CA | SER I | 182 | 1.272 | −24.214 | 26.503 | 1.00 | 53.52 | C | C |
| ATOM | 8188 | CB | SER I | 182 | 2.491 | −24.642 | 27.324 | 1.00 | 20.00 | C | C |
| ATOM | 8189 | OG | SER I | 182 | 2.601 | −23.890 | 28.521 | 1.00 | 20.00 | C | O |
| ATOM | 8190 | C | SER I | 182 | −0.025 | −24.557 | 27.236 | 1.00 | 44.76 | C | C |
| ATOM | 8191 | O | SER I | 182 | −0.492 | −23.802 | 28.090 | 1.00 | 37.73 | C | O |
| ATOM | 8192 | N | ASN I | 183 | −0.652 | −25.649 | 26.815 | 1.00 | 47.84 | C | N |
| ATOM | 8193 | CA | ASN I | 183 | −1.684 | −26.308 | 27.603 | 1.00 | 59.45 | C | C |
| ATOM | 8194 | CB | ASN I | 183 | −3.002 | −26.334 | 26.822 | 1.00 | 20.00 | C | C |
| ATOM | 8195 | CG | ASN I | 183 | −3.598 | −24.951 | 26.632 | 1.00 | 20.00 | C | C |
| ATOM | 8196 | OD1 | ASN I | 183 | −3.426 | −24.069 | 27.473 | 1.00 | 20.00 | C | O |
| ATOM | 8197 | ND2 | ASN I | 183 | −4.381 | −24.785 | 25.572 | 1.00 | 20.00 | C | N |
| ATOM | 8198 | C | ASN I | 183 | −1.232 | −27.732 | 27.905 | 1.00 | 75.41 | C | C |
| ATOM | 8199 | O | ASN I | 183 | −0.535 | −28.342 | 27.096 | 1.00 | 102.31 | C | O |
| ATOM | 8200 | N | GLY I | 184 | −1.489 | −28.200 | 29.121 | 1.00 | 79.30 | C | N |
| ATOM | 8201 | CA | GLY I | 184 | −1.001 | −29.510 | 29.549 | 1.00 | 88.92 | C | C |
| ATOM | 8202 | C | GLY I | 184 | −0.204 | −30.239 | 28.479 | 1.00 | 101.85 | C | C |
| ATOM | 8203 | O | GLY I | 184 | −0.764 | −30.990 | 27.680 | 1.00 | 113.48 | C | O |
| ATOM | 8204 | N | VAL I | 185 | 1.103 | −29.994 | 28.448 | 1.00 | 86.84 | C | N |
| ATOM | 8205 | CA | VAL I | 185 | 2.047 | −30.825 | 27.696 | 1.00 | 81.80 | C | C |
| ATOM | 8206 | CB | VAL I | 185 | 1.899 | −32.323 | 28.027 | 1.00 | 20.00 | C | C |
| ATOM | 8207 | CG1 | VAL I | 185 | 2.803 | −33.155 | 27.126 | 1.00 | 20.00 | C | C |
| ATOM | 8208 | CG2 | VAL I | 185 | 2.219 | −32.578 | 29.491 | 1.00 | 20.00 | C | C |
| ATOM | 8209 | C | VAL I | 185 | 1.997 | −30.638 | 26.179 | 1.00 | 81.69 | C | C |
| ATOM | 8210 | O | VAL I | 185 | 2.634 | −31.388 | 25.439 | 1.00 | 99.84 | C | O |
| ATOM | 8211 | N | SER I | 186 | 1.282 | −29.618 | 25.718 | 1.00 | 70.63 | C | N |
| ATOM | 8212 | CA | SER I | 186 | 1.132 | −29.398 | 24.284 | 1.00 | 72.29 | C | C |
| ATOM | 8213 | CB | SER I | 186 | −0.201 | −29.962 | 23.792 | 1.00 | 20.00 | C | C |
| ATOM | 8214 | OG | SER I | 186 | −1.291 | −29.357 | 24.467 | 1.00 | 20.00 | C | O |
| ATOM | 8215 | C | SER I | 186 | 1.247 | −27.923 | 23.915 | 1.00 | 62.99 | C | C |
| ATOM | 8216 | O | SER I | 186 | 0.649 | −27.064 | 24.565 | 1.00 | 58.35 | C | O |
| ATOM | 8217 | N | VAL I | 187 | 2.013 | −27.638 | 22.865 | 1.00 | 66.49 | C | N |
| ATOM | 8218 | CA | VAL I | 187 | 2.203 | −26.266 | 22.398 | 1.00 | 57.36 | C | C |
| ATOM | 8219 | CB | VAL I | 187 | 3.581 | −26.078 | 21.735 | 1.00 | 20.00 | C | C |
| ATOM | 8220 | CG1 | VAL I | 187 | 3.748 | −24.643 | 21.256 | 1.00 | 20.00 | C | C |
| ATOM | 8221 | CG2 | VAL I | 187 | 4.691 | −26.452 | 22.699 | 1.00 | 20.00 | C | C |
| ATOM | 8222 | C | VAL I | 187 | 1.116 | −25.863 | 21.405 | 1.00 | 53.67 | C | C |
| ATOM | 8223 | O | VAL I | 187 | 0.949 | −26.501 | 20.365 | 1.00 | 50.10 | C | O |
| ATOM | 8224 | N | LEU I | 188 | 0.381 | −24.803 | 21.731 | 1.00 | 43.20 | C | N |
| ATOM | 8225 | CA | LEU I | 188 | −0.676 | −24.302 | 20.858 | 1.00 | 41.76 | C | C |
| ATOM | 8226 | CB | LEU I | 188 | −1.749 | −23.580 | 21.674 | 1.00 | 20.00 | C | C |
| ATOM | 8227 | CG | LEU I | 188 | −2.557 | −24.455 | 22.632 | 1.00 | 20.00 | C | C |
| ATOM | 8228 | CD1 | LEU I | 188 | −3.533 | −23.609 | 23.429 | 1.00 | 20.00 | C | C |
| ATOM | 8229 | CD2 | LEU I | 188 | −3.287 | −25.542 | 21.865 | 1.00 | 20.00 | C | C |
| ATOM | 8230 | C | LEU I | 188 | −0.102 | −23.361 | 19.813 | 1.00 | 44.75 | C | C |
| ATOM | 8231 | O | LEU I | 188 | −0.674 | −23.184 | 18.738 | 1.00 | 45.28 | C | O |
| ATOM | 8232 | N | THR I | 189 | 1.054 | −22.786 | 20.124 | 1.00 | 39.28 | C | N |
| ATOM | 8233 | CA | THR I | 189 | 1.675 | −21.805 | 19.250 | 1.00 | 36.51 | C | C |
| ATOM | 8234 | CB | THR I | 189 | 0.614 | −20.957 | 18.526 | 1.00 | 20.00 | C | C |
| ATOM | 8235 | OG1 | THR I | 189 | −0.113 | −20.177 | 19.481 | 1.00 | 20.00 | C | O |
| ATOM | 8236 | CG2 | THR I | 189 | −0.360 | −21.858 | 17.785 | 1.00 | 20.00 | C | C |
| ATOM | 8237 | C | THR I | 189 | 2.615 | −20.901 | 20.039 | 1.00 | 45.83 | C | C |
| ATOM | 8238 | O | THR I | 189 | 3.150 | −21.304 | 21.072 | 1.00 | 42.57 | C | O |
| ATOM | 8239 | N | SER I | 190 | 2.853 | −19.698 | 19.530 | 1.00 | 40.44 | C | N |
| ATOM | 8240 | CA | SER I | 190 | 3.792 | −18.785 | 20.166 | 1.00 | 41.82 | C | C |
| ATOM | 8241 | CB | SER I | 190 | 5.022 | −18.584 | 19.283 | 1.00 | 20.00 | C | C |
| ATOM | 8242 | OG | SER I | 190 | 4.664 | −18.015 | 18.038 | 1.00 | 20.00 | C | O |
| ATOM | 8243 | C | SER I | 190 | 3.146 | −17.444 | 20.491 | 1.00 | 33.81 | C | C |
| ATOM | 8244 | O | SER I | 190 | 2.391 | −16.897 | 19.687 | 1.00 | 49.61 | C | O |
| ATOM | 8245 | N | LYS I | 191 | 3.454 | −16.920 | 21.674 | 1.00 | 37.37 | C | N |
| ATOM | 8246 | CA | LYS I | 191 | 2.986 | −15.598 | 22.086 | 1.00 | 41.23 | C | C |
| ATOM | 8247 | CB | LYS I | 191 | 2.358 | −15.664 | 23.478 | 1.00 | 20.00 | C | C |
| ATOM | 8248 | CG | LYS I | 191 | 1.069 | −16.459 | 23.525 | 1.00 | 20.00 | C | C |
| ATOM | 8249 | CD | LYS I | 191 | 0.505 | −16.514 | 24.931 | 1.00 | 20.00 | C | C |
| ATOM | 8250 | CE | LYS I | 191 | −0.626 | −17.531 | 25.037 | 1.00 | 20.00 | C | C |
| ATOM | 8251 | NZ | LYS I | 191 | −0.791 | −18.032 | 26.435 | 1.00 | 20.00 | C | N |
| ATOM | 8252 | C | LYS I | 191 | 4.112 | −14.568 | 22.069 | 1.00 | 35.03 | C | C |
| ATOM | 8253 | O | LYS I | 191 | 5.120 | −14.728 | 22.757 | 1.00 | 40.06 | C | O |
| ATOM | 8254 | N | VAL I | 192 | 3.920 | −13.495 | 21.308 | 1.00 | 33.70 | C | N |
| ATOM | 8255 | CA | VAL I | 192 | 4.967 | −12.498 | 21.120 | 1.00 | 27.61 | C | C |
| ATOM | 8256 | CB | VAL I | 192 | 5.392 | −12.392 | 19.646 | 1.00 | 20.00 | C | C |
| ATOM | 8257 | CG1 | VAL I | 192 | 6.395 | −11.267 | 19.465 | 1.00 | 20.00 | C | C |
| ATOM | 8258 | CG2 | VAL I | 192 | 5.972 | −13.709 | 19.165 | 1.00 | 20.00 | C | C |
| ATOM | 8259 | C | VAL I | 192 | 4.550 | −11.121 | 21.624 | 1.00 | 31.74 | C | C |
| ATOM | 8260 | O | VAL I | 192 | 3.376 | −10.755 | 21.573 | 1.00 | 25.58 | C | O |
| ATOM | 8261 | N | LEU I | 193 | 5.526 | −10.364 | 22.114 | 1.00 | 28.88 | C | N |
| ATOM | 8262 | CA | LEU I | 193 | 5.316 | −8.973 | 22.488 | 1.00 | 34.36 | C | C |
| ATOM | 8263 | CB | LEU I | 193 | 5.118 | −8.855 | 23.998 | 1.00 | 20.00 | C | C |
| ATOM | 8264 | CG | LEU I | 193 | 3.893 | −9.566 | 24.567 | 1.00 | 20.00 | C | C |
| ATOM | 8265 | CD1 | LEU I | 193 | 3.915 | −9.514 | 26.084 | 1.00 | 20.00 | C | C |
| ATOM | 8266 | CD2 | LEU I | 193 | 2.623 | −8.947 | 24.019 | 1.00 | 20.00 | C | C |
| ATOM | 8267 | C | LEU I | 193 | 6.507 | −8.126 | 22.059 | 1.00 | 33.03 | C | C |
| ATOM | 8268 | O | LEU I | 193 | 7.592 | −8.238 | 22.627 | 1.00 | 44.93 | C | O |
| ATOM | 8269 | N | ASP I | 194 | 6.304 | −7.294 | 21.042 | 1.00 | 35.09 | C | N |
| ATOM | 8270 | CA | ASP I | 194 | 7.409 | −6.702 | 20.288 | 1.00 | 41.39 | C | C |
| ATOM | 8271 | CB | ASP I | 194 | 6.892 | −6.027 | 19.014 | 1.00 | 20.00 | C | C |
| ATOM | 8272 | CG | ASP I | 194 | 6.375 | −7.023 | 17.994 | 1.00 | 20.00 | C | C |
| ATOM | 8273 | OD1 | ASP I | 194 | 6.659 | −8.229 | 18.141 | 1.00 | 20.00 | C | O |
| ATOM | 8274 | OD2 | ASP I | 194 | 5.654 | −6.606 | 17.064 | 1.00 | 20.00 | C | O |
| ATOM | 8275 | C | ASP I | 194 | 8.255 | −5.712 | 21.094 | 1.00 | 38.04 | C | C |
| ATOM | 8276 | O | ASP I | 194 | 9.349 | −5.339 | 20.670 | 1.00 | 48.03 | C | O |
| ATOM | 8277 | N | LEU I | 195 | 7.723 | −5.232 | 22.215 | 1.00 | 43.64 | C | N |
| ATOM | 8278 | CA | LEU I | 195 | 8.360 | −4.139 | 22.947 | 1.00 | 35.97 | C | C |
| ATOM | 8279 | CB | LEU I | 195 | 7.417 | −2.935 | 23.045 | 1.00 | 20.00 | C | C |
| ATOM | 8280 | CG | LEU I | 195 | 7.024 | −2.281 | 21.714 | 1.00 | 20.00 | C | C |
| ATOM | 8281 | CD1 | LEU I | 195 | 5.965 | −1.195 | 21.915 | 1.00 | 20.00 | C | C |
| ATOM | 8282 | CD2 | LEU I | 195 | 8.249 | −1.730 | 20.980 | 1.00 | 20.00 | C | C |
| ATOM | 8283 | C | LEU I | 195 | 8.858 | −4.546 | 24.335 | 1.00 | 43.63 | C | C |
| ATOM | 8284 | O | LEU I | 195 | 9.609 | −3.811 | 24.972 | 1.00 | 34.71 | C | O |
| ATOM | 8285 | N | ASN I | 196 | 8.535 | −5.769 | 24.742 | 1.00 | 44.30 | C | N |
| ATOM | 8286 | CA | ASN I | 196 | 8.824 | −6.238 | 26.095 | 1.00 | 42.38 | C | C |
| ATOM | 8287 | CB | ASN I | 196 | 8.399 | −7.701 | 26.254 | 1.00 | 20.00 | C | C |
| ATOM | 8288 | CG | ASN I | 196 | 6.892 | −7.874 | 26.221 | 1.00 | 20.00 | C | C |
| ATOM | 8289 | OD1 | ASN I | 196 | 6.146 | −6.952 | 26.545 | 1.00 | 20.00 | C | O |
| ATOM | 8290 | ND2 | ASN I | 196 | 6.438 | −9.055 | 25.817 | 1.00 | 20.00 | C | N |
| ATOM | 8291 | C | ASN I | 196 | 10.282 | −6.058 | 26.511 | 1.00 | 43.66 | C | C |
| ATOM | 8292 | O | ASN I | 196 | 10.568 | −5.610 | 27.622 | 1.00 | 33.62 | C | O |
| ATOM | 8293 | N | ASN I | 197 | 11.199 | −6.448 | 25.633 | 1.00 | 34.31 | C | N |
| ATOM | 8294 | CA | ASN I | 197 | 12.621 | −6.414 | 25.950 | 1.00 | 38.83 | C | C |
| ATOM | 8295 | CB | ASN I | 197 | 13.437 | −7.021 | 24.812 | 1.00 | 20.00 | C | C |
| ATOM | 8296 | CG | ASN I | 197 | 13.306 | −8.527 | 24.747 | 1.00 | 20.00 | C | C |
| ATOM | 8297 | OD1 | ASN I | 197 | 13.064 | −9.180 | 25.764 | 1.00 | 20.00 | C | O |
| ATOM | 8298 | ND2 | ASN I | 197 | 13.315 | −9.069 | 23.533 | 1.00 | 20.00 | C | N |
| ATOM | 8299 | C | ASN I | 197 | 13.106 | −5.005 | 26.239 | 1.00 | 39.90 | C | C |
| ATOM | 8300 | O | ASN I | 197 | 13.745 | −4.752 | 27.261 | 1.00 | 39.04 | C | O |
| ATOM | 8301 | N | TYR I | 198 | 12.824 | −4.095 | 25.314 | 1.00 | 35.09 | C | N |
| ATOM | 8302 | CA | TYR I | 198 | 13.109 | −2.682 | 25.518 | 1.00 | 41.03 | C | C |
| ATOM | 8303 | CB | TYR I | 198 | 12.610 | −1.859 | 24.332 | 1.00 | 20.00 | C | C |
| ATOM | 8304 | CG | TYR I | 198 | 13.298 | −2.189 | 23.031 | 1.00 | 20.00 | C | C |
| ATOM | 8305 | CD1 | TYR I | 198 | 14.497 | −1.579 | 22.687 | 1.00 | 20.00 | C | C |
| ATOM | 8306 | CE1 | TYR I | 198 | 15.110 | −1.847 | 21.488 | 1.00 | 20.00 | C | C |
| ATOM | 8307 | CZ | TYR I | 198 | 14.536 | −2.752 | 20.620 | 1.00 | 20.00 | C | C |
| ATOM | 8308 | OH | TYR I | 198 | 15.138 | −3.016 | 19.411 | 1.00 | 20.00 | C | O |
| ATOM | 8309 | CE2 | TYR I | 198 | 13.349 | −3.378 | 20.941 | 1.00 | 20.00 | C | C |
| ATOM | 8310 | CD2 | TYR I | 198 | 12.749 | −3.109 | 22.148 | 1.00 | 20.00 | C | C |
| ATOM | 8311 | C | TYR I | 198 | 12.490 | −2.163 | 26.811 | 1.00 | 43.65 | C | C |
| ATOM | 8312 | O | TYR I | 198 | 13.128 | −1.410 | 27.549 | 1.00 | 52.37 | C | O |
| ATOM | 8313 | N | ILE I | 199 | 11.279 | −2.622 | 27.115 | 1.00 | 35.86 | C | N |
| ATOM | 8314 | CA | ILE I | 199 | 10.573 | −2.185 | 28.316 | 1.00 | 45.39 | C | C |
| ATOM | 8315 | CB | ILE I | 199 | 9.108 | −2.624 | 28.306 | 1.00 | 20.00 | C | C |
| ATOM | 8316 | CG1 | ILE I | 199 | 8.330 | −1.808 | 27.272 | 1.00 | 20.00 | C | C |
| ATOM | 8317 | CD1 | ILE I | 199 | 7.307 | −2.607 | 26.505 | 1.00 | 20.00 | C | C |
| ATOM | 8318 | CG2 | ILE I | 199 | 8.502 | −2.461 | 29.691 | 1.00 | 20.00 | C | C |
| ATOM | 8319 | C | ILE I | 199 | 11.232 | −2.684 | 29.595 | 1.00 | 37.89 | C | C |
| ATOM | 8320 | O | ILE I | 199 | 11.344 | −1.943 | 30.572 | 1.00 | 45.28 | C | O |
| ATOM | 8321 | N | ASP I | 200 | 11.628 | −3.953 | 29.597 | 1.00 | 37.36 | C | N |
| ATOM | 8322 | CA | ASP I | 200 | 12.325 | −4.543 | 30.737 | 1.00 | 46.66 | C | C |
| ATOM | 8323 | CB | ASP I | 200 | 12.549 | −6.040 | 30.507 | 1.00 | 20.00 | C | C |
| ATOM | 8324 | CG | ASP I | 200 | 11.269 | −6.841 | 30.616 | 1.00 | 20.00 | C | C |
| ATOM | 8325 | OD1 | ASP I | 200 | 10.283 | −6.314 | 31.172 | 1.00 | 20.00 | C | O |
| ATOM | 8326 | OD2 | ASP I | 200 | 11.248 | −7.996 | 30.143 | 1.00 | 20.00 | C | O |
| ATOM | 8327 | C | ASP I | 200 | 13.655 | −3.842 | 31.033 | 1.00 | 51.05 | C | C |
| ATOM | 8328 | O | ASP I | 200 | 14.077 | −3.753 | 32.188 | 1.00 | 57.39 | C | O |
| ATOM | 8329 | N | LYS I | 201 | 14.284 | −3.306 | 29.989 | 1.00 | 57.68 | C | N |
| ATOM | 8330 | CA | LYS I | 201 | 15.560 | −2.605 | 30.126 | 1.00 | 44.02 | C | C |
| ATOM | 8331 | CB | LYS I | 201 | 16.228 | −2.417 | 28.760 | 1.00 | 20.00 | C | C |
| ATOM | 8332 | CG | LYS I | 201 | 16.590 | −3.716 | 28.055 | 1.00 | 20.00 | C | C |
| ATOM | 8333 | CD | LYS I | 201 | 17.363 | −3.463 | 26.764 | 1.00 | 20.00 | C | C |
| ATOM | 8334 | CE | LYS I | 201 | 17.644 | −4.764 | 26.018 | 1.00 | 20.00 | C | C |
| ATOM | 8335 | NZ | LYS I | 201 | 18.195 | −4.531 | 24.654 | 1.00 | 20.00 | C | N |
| ATOM | 8336 | C | LYS I | 201 | 15.409 | −1.254 | 30.827 | 1.00 | 43.56 | C | C |
| ATOM | 8337 | O | LYS I | 201 | 16.366 | −0.743 | 31.407 | 1.00 | 51.34 | C | O |
| ATOM | 8338 | N | GLN I | 202 | 14.202 | −0.694 | 30.795 | 1.00 | 46.06 | C | N |
| ATOM | 8339 | CA | GLN I | 202 | 13.987 | 0.689 | 31.223 | 1.00 | 44.95 | C | C |
| ATOM | 8340 | CB | GLN I | 202 | 13.366 | 1.518 | 30.101 | 1.00 | 20.00 | C | C |
| ATOM | 8341 | CG | GLN I | 202 | 14.344 | 1.932 | 29.024 | 1.00 | 20.00 | C | C |
| ATOM | 8342 | CD | GLN I | 202 | 13.660 | 2.551 | 27.823 | 1.00 | 20.00 | C | C |
| ATOM | 8343 | OE1 | GLN I | 202 | 12.435 | 2.519 | 27.702 | 1.00 | 20.00 | C | O |
| ATOM | 8344 | NE2 | GLN I | 202 | 14.452 | 3.135 | 26.932 | 1.00 | 20.00 | C | N |
| ATOM | 8345 | C | GLN I | 202 | 13.142 | 0.816 | 32.489 | 1.00 | 42.35 | C | C |
| ATOM | 8346 | O | GLN I | 202 | 13.166 | 1.853 | 33.151 | 1.00 | 47.00 | C | O |
| ATOM | 8347 | N | LEU I | 203 | 12.318 | −0.189 | 32.766 | 1.00 | 49.55 | C | N |
| ATOM | 8348 | CA | LEU I | 203 | 11.397 | −0.111 | 33.895 | 1.00 | 50.77 | C | C |
| ATOM | 8349 | CB | LEU I | 203 | 10.332 | −1.205 | 33.796 | 1.00 | 20.00 | C | C |
| ATOM | 8350 | CG | LEU I | 203 | 9.392 | −1.066 | 32.594 | 1.00 | 20.00 | C | C |
| ATOM | 8351 | CD1 | LEU I | 203 | 8.616 | −2.347 | 32.316 | 1.00 | 20.00 | C | C |
| ATOM | 8352 | CD2 | LEU I | 203 | 8.447 | 0.111 | 32.787 | 1.00 | 20.00 | C | C |
| ATOM | 8353 | C | LEU I | 203 | 12.134 | −0.184 | 35.231 | 1.00 | 52.51 | C | C |
| ATOM | 8354 | O | LEU I | 203 | 13.096 | −0.937 | 35.379 | 1.00 | 46.02 | C | O |
| ATOM | 8355 | N | LEU I | 204 | 11.747 | 0.686 | 36.159 | 1.00 | 51.45 | C | N |
| ATOM | 8356 | CA | LEU I | 204 | 12.308 | 0.682 | 37.507 | 1.00 | 58.44 | C | C |
| ATOM | 8357 | CB | LEU I | 204 | 12.055 | 2.021 | 38.187 | 1.00 | 20.00 | C | C |
| ATOM | 8358 | CG | LEU I | 204 | 12.780 | 3.206 | 37.565 | 1.00 | 20.00 | C | C |
| ATOM | 8359 | CD1 | LEU I | 204 | 12.380 | 4.480 | 38.291 | 1.00 | 20.00 | C | C |
| ATOM | 8360 | CD2 | LEU I | 204 | 14.286 | 2.982 | 37.637 | 1.00 | 20.00 | C | C |
| ATOM | 8361 | C | LEU I | 204 | 11.700 | −0.425 | 38.354 | 1.00 | 54.99 | C | C |
| ATOM | 8362 | O | LEU I | 204 | 10.489 | −0.633 | 38.319 | 1.00 | 55.13 | C | O |
| ATOM | 8363 | N | PRO I | 205 | 12.498 | −0.979 | 39.277 | 1.00 | 58.64 | C | N |
| ATOM | 8364 | CA | PRO I | 205 | 12.014 | −2.145 | 39.995 | 1.00 | 52.19 | C | C |
| ATOM | 8365 | CB | PRO I | 205 | 13.198 | −2.496 | 40.896 | 1.00 | 20.00 | C | C |
| ATOM | 8366 | CG | PRO I | 205 | 13.924 | −1.175 | 41.116 | 1.00 | 20.00 | C | C |
| ATOM | 8367 | CD | PRO I | 205 | 13.404 | −0.175 | 40.112 | 1.00 | 20.00 | C | C |
| ATOM | 8368 | C | PRO I | 205 | 10.799 | −1.765 | 40.840 | 1.00 | 54.30 | C | C |
| ATOM | 8369 | O | PRO I | 205 | 10.014 | −2.630 | 41.235 | 1.00 | 70.22 | C | O |
| ATOM | 8370 | N | ILE I | 206 | 10.656 | −0.465 | 41.096 | 1.00 | 62.70 | C | N |
| ATOM | 8371 | CA | ILE I | 206 | 9.491 | 0.087 | 41.787 | 1.00 | 68.19 | C | C |
| ATOM | 8372 | CB | ILE I | 206 | 9.725 | 0.181 | 43.311 | 1.00 | 20.00 | C | C |
| ATOM | 8373 | CG1 | ILE I | 206 | 9.753 | −1.214 | 43.941 | 1.00 | 20.00 | C | C |
| ATOM | 8374 | CD1 | ILE I | 206 | 10.200 | −1.220 | 45.389 | 1.00 | 20.00 | C | C |
| ATOM | 8375 | CG2 | ILE I | 206 | 8.656 | 1.047 | 43.966 | 1.00 | 20.00 | C | C |
| ATOM | 8376 | C | ILE I | 206 | 9.175 | 1.484 | 41.253 | 1.00 | 62.35 | C | C |
| ATOM | 8377 | O | ILE I | 206 | 10.081 | 2.268 | 40.973 | 1.00 | 67.16 | C | O |
| ATOM | 8378 | N | VAL I | 207 | 7.894 | 1.749 | 41.015 | 1.00 | 52.22 | C | N |
| ATOM | 8379 | CA | VAL I | 207 | 7.461 | 3.050 | 40.521 | 1.00 | 51.31 | C | C |
| ATOM | 8380 | CB | VAL I | 207 | 6.469 | 2.918 | 39.345 | 1.00 | 20.00 | C | C |
| ATOM | 8381 | CG1 | VAL I | 207 | 5.891 | 4.279 | 38.984 | 1.00 | 20.00 | C | C |
| ATOM | 8382 | CG2 | VAL I | 207 | 7.149 | 2.291 | 38.139 | 1.00 | 20.00 | C | C |
| ATOM | 8383 | C | VAL I | 207 | 6.800 | 3.846 | 41.634 | 1.00 | 62.09 | C | C |
| ATOM | 8384 | O | VAL I | 207 | 5.644 | 3.607 | 41.980 | 1.00 | 58.88 | C | O |
| ATOM | 8385 | N | ASN I | 208 | 7.557 | 4.759 | 42.229 | 1.00 | 71.72 | C | N |
| ATOM | 8386 | CA | ASN I | 208 | 7.023 | 5.625 | 43.267 | 1.00 | 70.92 | C | C |
| ATOM | 8387 | CB | ASN I | 208 | 8.004 | 5.721 | 44.433 | 1.00 | 20.00 | C | C |
| ATOM | 8388 | CG | ASN I | 208 | 8.100 | 4.428 | 45.222 | 1.00 | 20.00 | C | C |
| ATOM | 8389 | OD1 | ASN I | 208 | 7.170 | 3.620 | 45.228 | 1.00 | 20.00 | C | O |
| ATOM | 8390 | ND2 | ASN I | 208 | 9.234 | 4.221 | 45.884 | 1.00 | 20.00 | C | N |
| ATOM | 8391 | C | ASN I | 208 | 6.699 | 7.015 | 42.735 | 1.00 | 63.05 | C | C |
| ATOM | 8392 | O | ASN I | 208 | 6.780 | 7.264 | 41.532 | 1.00 | 61.34 | C | O |
| ATOM | 8393 | N | LYS I | 209 | 6.391 | 7.933 | 43.645 | 1.00 | 68.91 | C | N |
| ATOM | 8394 | CA | LYS I | 209 | 5.953 | 9.273 | 43.274 | 1.00 | 59.11 | C | C |
| ATOM | 8395 | CB | LYS I | 209 | 5.349 | 9.992 | 44.482 | 1.00 | 20.00 | C | C |
| ATOM | 8396 | CG | LYS I | 209 | 4.044 | 9.391 | 44.979 | 1.00 | 20.00 | C | C |
| ATOM | 8397 | CD | LYS I | 209 | 3.507 | 10.158 | 46.179 | 1.00 | 20.00 | C | C |
| ATOM | 8398 | CE | LYS I | 209 | 2.159 | 9.612 | 46.629 | 1.00 | 20.00 | C | C |
| ATOM | 8399 | NZ | LYS I | 209 | 1.583 | 10.385 | 47.765 | 1.00 | 20.00 | C | N |
| ATOM | 8400 | C | LYS I | 209 | 7.100 | 10.100 | 42.702 | 1.00 | 50.69 | C | C |
| ATOM | 8401 | O | LYS I | 209 | 6.883 | 11.178 | 42.152 | 1.00 | 51.01 | C | O |
| ATOM | 8402 | N | GLN I | 210 | 8.326 | 9.638 | 42.920 | 1.00 | 44.68 | C | N |
| ATOM | 8403 | CA | GLN I | 210 | 9.495 | 10.293 | 42.349 | 1.00 | 49.52 | C | C |
| ATOM | 8404 | CB | GLN I | 210 | 10.724 | 10.052 | 43.223 | 1.00 | 20.00 | C | C |
| ATOM | 8405 | CG | GLN I | 210 | 10.667 | 10.739 | 44.573 | 1.00 | 20.00 | C | C |
| ATOM | 8406 | CD | GLN I | 210 | 11.865 | 10.409 | 45.435 | 1.00 | 20.00 | C | C |
| ATOM | 8407 | OE1 | GLN I | 210 | 12.517 | 9.385 | 45.239 | 1.00 | 20.00 | C | O |
| ATOM | 8408 | NE2 | GLN I | 210 | 12.173 | 11.286 | 46.383 | 1.00 | 20.00 | C | N |
| ATOM | 8409 | C | GLN I | 210 | 9.755 | 9.786 | 40.938 | 1.00 | 62.08 | C | C |
| ATOM | 8410 | O | GLN I | 210 | 9.786 | 10.560 | 39.983 | 1.00 | 80.43 | C | O |
| ATOM | 8411 | N | SER I | 211 | 9.902 | 8.472 | 40.813 | 1.00 | 58.92 | C | N |
| ATOM | 8412 | CA | SER I | 211 | 10.076 | 7.840 | 39.514 | 1.00 | 47.88 | C | C |
| ATOM | 8413 | CB | SER I | 211 | 9.987 | 6.319 | 39.647 | 1.00 | 20.00 | C | C |
| ATOM | 8414 | OG | SER I | 211 | 8.735 | 5.930 | 40.184 | 1.00 | 20.00 | C | O |
| ATOM | 8415 | C | SER I | 211 | 9.050 | 8.344 | 38.503 | 1.00 | 57.32 | C | C |
| ATOM | 8416 | O | SER I | 211 | 9.372 | 8.538 | 37.331 | 1.00 | 50.74 | C | O |
| ATOM | 8417 | N | CYS I | 212 | 7.845 | 8.647 | 38.979 | 1.00 | 44.34 | C | N |
| ATOM | 8418 | CA | CYS I | 212 | 6.690 | 8.758 | 38.095 | 1.00 | 57.52 | C | C |
| ATOM | 8419 | CB | CYS I | 212 | 5.453 | 9.307 | 38.834 | 1.00 | 20.00 | C | C |
| ATOM | 8420 | SG | CYS I | 212 | 4.771 | 8.198 | 40.141 | 1.00 | 20.00 | C | S |
| ATOM | 8421 | C | CYS I | 212 | 7.005 | 9.537 | 36.808 | 1.00 | 60.91 | C | C |
| ATOM | 8422 | O | CYS I | 212 | 6.991 | 8.952 | 35.727 | 1.00 | 52.17 | C | O |
| ATOM | 8423 | N | SER I | 213 | 7.305 | 10.827 | 36.916 | 1.00 | 21.55 | C | N |
| ATOM | 8424 | CA | SER I | 213 | 7.501 | 11.624 | 35.711 | 1.00 | 21.67 | C | C |
| ATOM | 8425 | CB | SER I | 213 | 7.747 | 13.090 | 36.075 | 1.00 | 21.81 | C | C |
| ATOM | 8426 | OG | SER I | 213 | 6.648 | 13.630 | 36.788 | 1.00 | 23.00 | C | O |
| ATOM | 8427 | C | SER I | 213 | 8.650 | 11.098 | 34.858 | 1.00 | 21.45 | C | C |
| ATOM | 8428 | O | SER I | 213 | 8.509 | 10.969 | 33.640 | 1.00 | 21.98 | C | O |
| ATOM | 8429 | N | ILE I | 214 | 9.775 | 10.772 | 35.484 | 1.00 | 20.90 | C | N |
| ATOM | 8430 | CA | ILE I | 214 | 10.875 | 10.168 | 34.746 | 1.00 | 20.45 | C | C |
| ATOM | 8431 | CB | ILE I | 214 | 12.127 | 10.002 | 35.624 | 1.00 | 20.53 | C | C |
| ATOM | 8432 | CG1 | ILE I | 214 | 12.599 | 11.363 | 36.141 | 1.00 | 20.46 | C | C |
| ATOM | 8433 | CD1 | ILE I | 214 | 13.831 | 11.289 | 37.016 | 1.00 | 21.75 | C | C |
| ATOM | 8434 | CG2 | ILE I | 214 | 13.235 | 9.309 | 34.846 | 1.00 | 20.34 | C | C |
| ATOM | 8435 | C | ILE I | 214 | 10.423 | 8.802 | 34.247 | 1.00 | 20.15 | C | C |
| ATOM | 8436 | O | ILE I | 214 | 10.647 | 8.427 | 33.090 | 1.00 | 19.91 | C | O |
| ATOM | 8437 | N | SER I | 215 | 9.751 | 8.075 | 35.136 | 1.00 | 19.84 | C | N |
| ATOM | 8438 | CA | SER I | 215 | 9.240 | 6.749 | 34.827 | 1.00 | 19.58 | C | C |
| ATOM | 8439 | CB | SER I | 215 | 8.632 | 6.104 | 36.073 | 1.00 | 19.76 | C | C |
| ATOM | 8440 | OG | SER I | 215 | 8.132 | 4.810 | 35.785 | 1.00 | 19.97 | C | O |
| ATOM | 8441 | C | SER I | 215 | 8.201 | 6.841 | 33.724 | 1.00 | 19.34 | C | C |
| ATOM | 8442 | O | SER I | 215 | 8.172 | 6.013 | 32.818 | 1.00 | 19.54 | C | O |
| ATOM | 8443 | N | ASN I | 216 | 7.351 | 7.858 | 33.804 | 1.00 | 18.80 | C | N |
| ATOM | 8444 | CA | ASN I | 216 | 6.324 | 8.059 | 32.796 | 1.00 | 18.36 | C | C |
| ATOM | 8445 | CB | ASN I | 216 | 5.406 | 9.218 | 33.187 | 1.00 | 18.41 | C | C |
| ATOM | 8446 | CG | ASN I | 216 | 4.701 | 8.983 | 34.508 | 1.00 | 19.55 | C | C |
| ATOM | 8447 | OD1 | ASN I | 216 | 3.922 | 9.819 | 34.967 | 1.00 | 20.52 | C | O |
| ATOM | 8448 | ND2 | ASN I | 216 | 4.971 | 7.840 | 35.128 | 1.00 | 20.43 | C | N |
| ATOM | 8449 | C | ASN I | 216 | 6.958 | 8.324 | 31.440 | 1.00 | 17.78 | C | C |
| ATOM | 8450 | O | ASN I | 216 | 6.509 | 7.800 | 30.421 | 1.00 | 17.91 | C | O |
| ATOM | 8451 | N | ILE I | 217 | 8.016 | 9.130 | 31.433 | 1.00 | 17.02 | C | N |
| ATOM | 8452 | CA | ILE I | 217 | 8.714 | 9.439 | 30.194 | 1.00 | 16.60 | C | C |
| ATOM | 8453 | CB | ILE I | 217 | 9.833 | 10.472 | 30.417 | 1.00 | 16.46 | C | C |
| ATOM | 8454 | CG1 | ILE I | 217 | 9.252 | 11.776 | 30.969 | 1.00 | 16.26 | C | C |
| ATOM | 8455 | CD1 | ILE I | 217 | 10.289 | 12.849 | 31.217 | 1.00 | 16.44 | C | C |
| ATOM | 8456 | CG2 | ILE I | 217 | 10.589 | 10.727 | 29.122 | 1.00 | 16.70 | C | C |
| ATOM | 8457 | C | ILE I | 217 | 9.314 | 8.168 | 29.609 | 1.00 | 16.73 | C | C |
| ATOM | 8458 | O | ILE I | 217 | 9.247 | 7.931 | 28.398 | 1.00 | 16.80 | C | O |
| ATOM | 8459 | N | GLU I | 218 | 9.885 | 7.339 | 30.478 | 1.00 | 17.02 | C | N |
| ATOM | 8460 | CA | GLU I | 218 | 10.485 | 6.090 | 30.031 | 1.00 | 17.70 | C | C |
| ATOM | 8461 | CB | GLU I | 218 | 11.172 | 5.376 | 31.197 | 1.00 | 18.20 | C | C |
| ATOM | 8462 | CG | GLU I | 218 | 12.289 | 6.178 | 31.844 | 1.00 | 21.31 | C | C |
| ATOM | 8463 | CD | GLU I | 218 | 12.951 | 5.437 | 32.989 | 1.00 | 25.72 | C | C |
| ATOM | 8464 | OE1 | GLU I | 218 | 13.884 | 5.998 | 33.602 | 1.00 | 26.96 | C | O |
| ATOM | 8465 | OE2 | GLU I | 218 | 12.538 | 4.294 | 33.277 | 1.00 | 27.07 | C | O |
| ATOM | 8466 | C | GLU I | 218 | 9.419 | 5.192 | 29.418 | 1.00 | 17.29 | C | C |
| ATOM | 8467 | O | GLU I | 218 | 9.642 | 4.555 | 28.387 | 1.00 | 17.62 | C | O |
| ATOM | 8468 | N | THR I | 219 | 8.253 | 5.156 | 30.055 | 1.00 | 16.62 | C | N |
| ATOM | 8469 | CA | THR I | 219 | 7.146 | 4.342 | 29.577 | 1.00 | 15.95 | C | C |
| ATOM | 8470 | CB | THR I | 219 | 5.952 | 4.387 | 30.547 | 1.00 | 15.95 | C | C |
| ATOM | 8471 | OG1 | THR I | 219 | 6.356 | 3.894 | 31.831 | 1.00 | 15.63 | C | O |
| ATOM | 8472 | CG2 | THR I | 219 | 4.806 | 3.536 | 30.021 | 1.00 | 15.97 | C | C |
| ATOM | 8473 | C | THR I | 219 | 6.696 | 4.825 | 28.207 | 1.00 | 15.64 | C | C |
| ATOM | 8474 | O | THR I | 219 | 6.414 | 4.022 | 27.321 | 1.00 | 15.93 | C | O |
| ATOM | 8475 | N | VAL I | 220 | 6.639 | 6.141 | 28.037 | 1.00 | 14.98 | C | N |
| ATOM | 8476 | CA | VAL I | 220 | 6.235 | 6.720 | 26.764 | 1.00 | 14.51 | C | C |
| ATOM | 8477 | CB | VAL I | 220 | 6.143 | 8.254 | 26.844 | 1.00 | 14.29 | C | C |
| ATOM | 8478 | CG1 | VAL I | 220 | 5.716 | 8.830 | 25.502 | 1.00 | 14.24 | C | C |
| ATOM | 8479 | CG2 | VAL I | 220 | 5.176 | 8.671 | 27.942 | 1.00 | 14.10 | C | C |
| ATOM | 8480 | C | VAL I | 220 | 7.233 | 6.334 | 25.682 | 1.00 | 14.64 | C | C |
| ATOM | 8481 | O | VAL I | 220 | 6.850 | 5.992 | 24.560 | 1.00 | 14.79 | C | O |
| ATOM | 8482 | N | ILE I | 221 | 8.516 | 6.375 | 26.028 | 1.00 | 14.43 | C | N |
| ATOM | 8483 | CA | ILE I | 221 | 9.556 | 6.013 | 25.075 | 1.00 | 14.36 | C | C |
| ATOM | 8484 | CB | ILE I | 221 | 10.962 | 6.235 | 25.661 | 1.00 | 14.03 | C | C |
| ATOM | 8485 | CG1 | ILE I | 221 | 11.156 | 7.704 | 26.041 | 1.00 | 13.72 | C | C |
| ATOM | 8486 | CD1 | ILE I | 221 | 11.011 | 8.660 | 24.877 | 1.00 | 14.17 | C | C |
| ATOM | 8487 | CG2 | ILE I | 221 | 12.027 | 5.791 | 24.671 | 1.00 | 13.91 | C | C |
| ATOM | 8488 | C | ILE I | 221 | 9.404 | 4.552 | 24.672 | 1.00 | 14.82 | C | C |
| ATOM | 8489 | O | ILE I | 221 | 9.531 | 4.204 | 23.496 | 1.00 | 14.82 | C | O |
| ATOM | 8490 | N | GLU I | 222 | 9.112 | 3.703 | 25.652 | 1.00 | 15.51 | C | N |
| ATOM | 8491 | CA | GLU I | 222 | 8.936 | 2.281 | 25.392 | 1.00 | 16.52 | C | C |
| ATOM | 8492 | CB | GLU I | 222 | 8.730 | 1.517 | 26.701 | 1.00 | 16.80 | C | C |
| ATOM | 8493 | CG | GLU I | 222 | 9.889 | 1.635 | 27.677 | 1.00 | 19.14 | C | C |
| ATOM | 8494 | CD | GLU I | 222 | 9.653 | 0.861 | 28.959 | 1.00 | 22.62 | C | C |
| ATOM | 8495 | OE1 | GLU I | 222 | 10.539 | 0.882 | 29.840 | 1.00 | 24.35 | C | O |
| ATOM | 8496 | OE2 | GLU I | 222 | 8.583 | 0.230 | 29.086 | 1.00 | 23.98 | C | O |
| ATOM | 8497 | C | GLU I | 222 | 7.750 | 2.064 | 24.464 | 1.00 | 16.65 | C | C |
| ATOM | 8498 | O | GLU I | 222 | 7.802 | 1.238 | 23.553 | 1.00 | 16.95 | C | O |
| ATOM | 8499 | N | PHE I | 223 | 6.681 | 2.817 | 24.701 | 1.00 | 16.70 | C | N |
| ATOM | 8500 | CA | PHE I | 223 | 5.482 | 2.717 | 23.885 | 1.00 | 16.95 | C | C |
| ATOM | 8501 | CB | PHE I | 223 | 4.370 | 3.600 | 24.453 | 1.00 | 16.85 | C | C |
| ATOM | 8502 | CG | PHE I | 223 | 3.058 | 3.460 | 23.736 | 1.00 | 17.23 | C | C |
| ATOM | 8503 | CD1 | PHE I | 223 | 2.127 | 2.519 | 24.145 | 1.00 | 17.98 | C | C |
| ATOM | 8504 | CE1 | PHE I | 223 | 0.919 | 2.387 | 23.488 | 1.00 | 18.28 | C | C |
| ATOM | 8505 | CZ | PHE I | 223 | 0.629 | 3.199 | 22.409 | 1.00 | 18.19 | C | C |
| ATOM | 8506 | CE2 | PHE I | 223 | 1.548 | 4.140 | 21.991 | 1.00 | 18.14 | C | C |
| ATOM | 8507 | CD2 | PHE I | 223 | 2.755 | 4.267 | 22.653 | 1.00 | 17.75 | C | C |
| ATOM | 8508 | C | PHE I | 223 | 5.790 | 3.119 | 22.450 | 1.00 | 17.28 | C | C |
| ATOM | 8509 | O | PHE I | 223 | 5.324 | 2.482 | 21.506 | 1.00 | 17.17 | C | O |
| ATOM | 8510 | N | GLN I | 224 | 6.581 | 4.175 | 22.287 | 1.00 | 17.62 | C | N |
| ATOM | 8511 | CA | GLN I | 224 | 6.954 | 4.619 | 20.952 | 1.00 | 18.16 | C | C |
| ATOM | 8512 | CB | GLN I | 224 | 7.759 | 5.918 | 21.021 | 1.00 | 18.51 | C | C |
| ATOM | 8513 | CG | GLN I | 224 | 7.015 | 7.075 | 21.669 | 1.00 | 20.06 | C | C |
| ATOM | 8514 | CD | GLN I | 224 | 7.842 | 8.344 | 21.718 | 1.00 | 22.03 | C | C |
| ATOM | 8515 | OE1 | GLN I | 224 | 7.387 | 9.376 | 22.214 | 1.00 | 22.15 | C | O |
| ATOM | 8516 | NE2 | GLN I | 224 | 9.064 | 8.275 | 21.203 | 1.00 | 22.11 | C | N |
| ATOM | 8517 | C | GLN I | 224 | 7.767 | 3.532 | 20.261 | 1.00 | 18.04 | C | C |
| ATOM | 8518 | O | GLN I | 224 | 7.561 | 3.238 | 19.083 | 1.00 | 18.17 | C | O |
| ATOM | 8519 | N | GLN I | 225 | 8.688 | 2.933 | 21.009 | 1.00 | 17.90 | C | N |
| ATOM | 8520 | CA | GLN I | 225 | 9.508 | 1.840 | 20.500 | 1.00 | 17.95 | C | C |
| ATOM | 8521 | CB | GLN I | 225 | 10.599 | 1.474 | 21.509 | 1.00 | 18.13 | C | C |
| ATOM | 8522 | CG | GLN I | 225 | 11.551 | 2.613 | 21.833 | 1.00 | 19.04 | C | C |
| ATOM | 8523 | CD | GLN I | 225 | 12.619 | 2.213 | 22.831 | 1.00 | 21.22 | C | C |
| ATOM | 8524 | OE1 | GLN I | 225 | 12.659 | 1.072 | 23.292 | 1.00 | 22.55 | C | O |
| ATOM | 8525 | NE2 | GLN I | 225 | 13.493 | 3.154 | 23.172 | 1.00 | 22.48 | C | N |
| ATOM | 8526 | C | GLN I | 225 | 8.664 | 0.611 | 20.172 | 1.00 | 17.84 | C | C |
| ATOM | 8527 | O | GLN I | 225 | 8.892 | −0.068 | 19.171 | 1.00 | 17.71 | C | O |
| ATOM | 8528 | N | LYS I | 226 | 7.693 | 0.334 | 21.036 | 1.00 | 17.95 | C | N |
| ATOM | 8529 | CA | LYS I | 226 | 6.848 | −0.831 | 20.912 | 1.00 | 18.00 | C | C |
| ATOM | 8530 | CB | LYS I | 226 | 6.821 | −1.593 | 22.234 | 1.00 | 18.21 | C | C |
| ATOM | 8531 | CG | LYS I | 226 | 8.189 | −1.697 | 22.905 | 1.00 | 18.50 | C | C |
| ATOM | 8532 | CD | LYS I | 226 | 8.132 | −2.591 | 24.132 | 1.00 | 19.87 | C | C |
| ATOM | 8533 | CE | LYS I | 226 | 9.357 | −2.411 | 25.004 | 1.00 | 21.36 | C | C |
| ATOM | 8534 | NZ | LYS I | 226 | 9.268 | −3.230 | 26.248 | 1.00 | 21.83 | C | N |
| ATOM | 8535 | C | LYS I | 226 | 5.446 | −0.408 | 20.499 | 1.00 | 17.84 | C | C |
| ATOM | 8536 | O | LYS I | 226 | 4.672 | −1.220 | 19.998 | 1.00 | 17.94 | C | O |
| ATOM | 8537 | N | ASN I | 227 | 5.209 | 0.900 | 20.514 | 1.00 | 17.70 | C | N |
| ATOM | 8538 | CA | ASN I | 227 | 4.173 | 1.494 | 19.683 | 1.00 | 17.66 | C | C |
| ATOM | 8539 | CB | ASN I | 227 | 3.153 | 2.256 | 20.549 | 1.00 | 17.36 | C | C |
| ATOM | 8540 | CG | ASN I | 227 | 2.612 | 1.411 | 21.710 | 1.00 | 17.40 | C | C |
| ATOM | 8541 | OD1 | ASN I | 227 | 2.451 | 0.196 | 21.589 | 1.00 | 17.84 | C | O |
| ATOM | 8542 | ND2 | ASN I | 227 | 2.333 | 2.059 | 22.834 | 1.00 | 17.22 | C | N |
| ATOM | 8543 | C | ASN I | 227 | 4.713 | 2.343 | 18.506 | 1.00 | 17.76 | C | C |
| ATOM | 8544 | O | ASN I | 227 | 3.943 | 2.750 | 17.635 | 1.00 | 17.92 | C | O |
| ATOM | 8545 | N | ASN I | 228 | 6.045 | 2.460 | 18.398 | 1.00 | 17.78 | C | N |
| ATOM | 8546 | CA | ASN I | 228 | 6.734 | 2.532 | 17.084 | 1.00 | 17.59 | C | C |
| ATOM | 8547 | CB | ASN I | 228 | 8.170 | 3.085 | 17.155 | 1.00 | 17.89 | C | C |
| ATOM | 8548 | CG | ASN I | 228 | 8.870 | 3.082 | 15.779 | 1.00 | 18.43 | C | C |
| ATOM | 8549 | OD1 | ASN I | 228 | 9.451 | 2.077 | 15.366 | 1.00 | 18.74 | C | O |
| ATOM | 8550 | ND2 | ASN I | 228 | 8.682 | 4.157 | 15.022 | 1.00 | 18.61 | C | N |
| ATOM | 8551 | C | ASN I | 228 | 6.765 | 1.204 | 16.358 | 1.00 | 17.09 | C | C |
| ATOM | 8552 | O | ASN I | 228 | 6.004 | 0.992 | 15.417 | 1.00 | 17.17 | C | O |
| ATOM | 8553 | N | ARG I | 229 | 7.685 | 0.328 | 16.745 | 1.00 | 16.33 | C | N |
| ATOM | 8554 | CA | ARG I | 229 | 7.664 | −1.016 | 16.195 | 1.00 | 15.76 | C | C |
| ATOM | 8555 | CB | ARG I | 229 | 8.624 | −1.952 | 16.933 | 1.00 | 15.70 | C | C |
| ATOM | 8556 | CG | ARG I | 229 | 9.369 | −2.904 | 16.000 | 1.00 | 14.99 | C | C |
| ATOM | 8557 | CD | ARG I | 229 | 9.887 | −4.135 | 16.728 | 1.00 | 15.33 | C | C |
| ATOM | 8558 | NE | ARG I | 229 | 9.601 | −5.376 | 16.008 | 1.00 | 15.59 | C | N |
| ATOM | 8559 | CZ | ARG I | 229 | 9.926 | −6.587 | 16.451 | 1.00 | 15.85 | C | C |
| ATOM | 8560 | NH1 | ARG I | 229 | 10.559 | −6.725 | 17.609 | 1.00 | 16.20 | C | N |
| ATOM | 8561 | NH2 | ARG I | 229 | 9.650 | −7.660 | 15.722 | 1.00 | 16.63 | C | N |
| ATOM | 8562 | C | ARG I | 229 | 6.238 | −1.559 | 16.223 | 1.00 | 15.62 | C | C |
| ATOM | 8563 | O | ARG I | 229 | 5.666 | −1.860 | 15.175 | 1.00 | 15.73 | C | O |
| ATOM | 8564 | N | LEU I | 230 | 5.601 | −1.468 | 17.387 | 1.00 | 15.46 | C | N |
| ATOM | 8565 | CA | LEU I | 230 | 4.191 | −1.829 | 17.511 | 1.00 | 15.22 | C | C |
| ATOM | 8566 | CB | LEU I | 230 | 3.735 | −1.792 | 18.974 | 1.00 | 15.08 | C | C |
| ATOM | 8567 | CG | LEU I | 230 | 2.252 | −2.079 | 19.224 | 1.00 | 14.70 | C | C |
| ATOM | 8568 | CD1 | LEU I | 230 | 2.003 | −3.572 | 19.359 | 1.00 | 15.19 | C | C |
| ATOM | 8569 | CD2 | LEU I | 230 | 1.743 | −1.335 | 20.449 | 1.00 | 15.46 | C | C |
| ATOM | 8570 | C | LEU I | 230 | 3.302 | −0.931 | 16.655 | 1.00 | 15.31 | C | C |
| ATOM | 8571 | O | LEU I | 230 | 2.417 | −1.415 | 15.950 | 1.00 | 15.46 | C | O |
| ATOM | 8572 | N | LEU I | 231 | 3.519 | 0.377 | 16.745 | 1.00 | 15.48 | C | N |
| ATOM | 8573 | CA | LEU I | 231 | 2.678 | 1.338 | 16.038 | 1.00 | 15.75 | C | C |
| ATOM | 8574 | CB | LEU I | 231 | 3.187 | 2.763 | 16.257 | 1.00 | 15.90 | C | C |
| ATOM | 8575 | CG | LEU I | 231 | 2.906 | 3.379 | 17.628 | 1.00 | 16.63 | C | C |
| ATOM | 8576 | CD1 | LEU I | 231 | 3.382 | 4.819 | 17.676 | 1.00 | 18.45 | C | C |
| ATOM | 8577 | CD2 | LEU I | 231 | 1.425 | 3.292 | 17.961 | 1.00 | 16.67 | C | C |
| ATOM | 8578 | C | LEU I | 231 | 2.653 | 1.025 | 14.551 | 1.00 | 15.76 | C | C |
| ATOM | 8579 | O | LEU I | 231 | 1.606 | 1.092 | 13.908 | 1.00 | 15.84 | C | O |
| ATOM | 8580 | N | GLU I | 232 | 3.828 | 0.750 | 13.997 | 1.00 | 15.71 | C | N |
| ATOM | 8581 | CA | GLU I | 232 | 3.973 | 0.563 | 12.562 | 1.00 | 15.93 | C | C |
| ATOM | 8582 | CB | GLU I | 232 | 5.447 | 0.626 | 12.163 | 1.00 | 16.15 | C | C |
| ATOM | 8583 | CG | GLU I | 232 | 6.142 | 1.913 | 12.568 | 1.00 | 18.59 | C | C |
| ATOM | 8584 | CD | GLU I | 232 | 6.407 | 2.823 | 11.387 | 1.00 | 21.47 | C | C |
| ATOM | 8585 | OE1 | GLU I | 232 | 5.892 | 2.532 | 10.286 | 1.00 | 22.14 | C | O |
| ATOM | 8586 | OE2 | GLU I | 232 | 7.214 | 3.765 | 11.530 | 1.00 | 23.34 | C | O |
| ATOM | 8587 | C | GLU I | 232 | 3.377 | −0.767 | 12.124 | 1.00 | 15.45 | C | C |
| ATOM | 8588 | O | GLU I | 232 | 2.921 | −0.908 | 10.990 | 1.00 | 15.95 | C | O |
| ATOM | 8589 | N | ILE I | 233 | 3.432 | −1.757 | 13.007 | 1.00 | 14.54 | C | N |
| ATOM | 8590 | CA | ILE I | 233 | 2.713 | −3.000 | 12.776 | 1.00 | 13.69 | C | C |
| ATOM | 8591 | CB | ILE I | 233 | 2.998 | −4.028 | 13.873 | 1.00 | 13.63 | C | C |
| ATOM | 8592 | CG1 | ILE I | 233 | 4.500 | −4.093 | 14.151 | 1.00 | 13.58 | C | C |
| ATOM | 8593 | CD1 | ILE I | 233 | 4.854 | −4.786 | 15.444 | 1.00 | 13.55 | C | C |
| ATOM | 8594 | CG2 | ILE I | 233 | 2.458 | −5.392 | 13.469 | 1.00 | 13.88 | C | C |
| ATOM | 8595 | C | ILE I | 233 | 1.219 | −2.728 | 12.735 | 1.00 | 13.28 | C | C |
| ATOM | 8596 | O | ILE I | 233 | 0.528 | −3.137 | 11.802 | 1.00 | 13.04 | C | O |
| ATOM | 8597 | N | THR I | 234 | 0.764 | −1.898 | 13.667 | 1.00 | 13.18 | C | N |
| ATOM | 8598 | CA | THR I | 234 | −0.651 | −1.590 | 13.796 | 1.00 | 13.39 | C | C |
| ATOM | 8599 | CB | THR I | 234 | −0.913 | −0.636 | 14.974 | 1.00 | 13.21 | C | C |
| ATOM | 8600 | OG1 | THR I | 234 | −0.476 | −1.248 | 16.193 | 1.00 | 13.15 | C | O |
| ATOM | 8601 | CG2 | THR I | 234 | −2.395 | −0.311 | 15.078 | 1.00 | 13.22 | C | C |
| ATOM | 8602 | C | THR I | 234 | −1.208 | −0.981 | 12.514 | 1.00 | 13.76 | C | C |
| ATOM | 8603 | O | THR I | 234 | −2.230 | −1.432 | 11.997 | 1.00 | 13.95 | C | O |
| ATOM | 8604 | N | ARG I | 235 | −0.535 | 0.045 | 12.003 | 1.00 | 14.26 | C | N |
| ATOM | 8605 | CA | ARG I | 235 | −0.945 | 0.670 | 10.752 | 1.00 | 14.92 | C | C |
| ATOM | 8606 | CB | ARG I | 235 | −0.051 | 1.874 | 10.432 | 1.00 | 15.30 | C | C |
| ATOM | 8607 | CG | ARG I | 235 | 0.875 | 1.681 | 9.240 | 1.00 | 16.27 | C | C |
| ATOM | 8608 | CD | ARG I | 235 | 2.190 | 2.436 | 9.413 | 1.00 | 18.52 | C | C |
| ATOM | 8609 | NE | ARG I | 235 | 2.083 | 3.558 | 10.344 | 1.00 | 17.86 | C | N |
| ATOM | 8610 | CZ | ARG I | 235 | 1.516 | 4.724 | 10.051 | 1.00 | 16.91 | C | C |
| ATOM | 8611 | NH1 | ARG I | 235 | 0.855 | 4.874 | 8.912 | 1.00 | 16.59 | C | N |
| ATOM | 8612 | NH2 | ARG I | 235 | 1.511 | 5.703 | 10.946 | 1.00 | 16.86 | C | N |
| ATOM | 8613 | C | ARG I | 235 | −0.904 | −0.353 | 9.620 | 1.00 | 15.05 | C | C |
| ATOM | 8614 | O | ARG I | 235 | −1.939 | −0.693 | 9.045 | 1.00 | 15.15 | C | O |
| ATOM | 8615 | N | GLU I | 236 | 0.240 | −1.020 | 9.504 | 1.00 | 15.18 | C | N |
| ATOM | 8616 | CA | GLU I | 236 | 0.451 | −2.047 | 8.490 | 1.00 | 15.24 | C | C |
| ATOM | 8617 | CB | GLU I | 236 | 1.725 | −2.835 | 8.801 | 1.00 | 15.69 | C | C |
| ATOM | 8618 | CG | GLU I | 236 | 2.432 | −3.396 | 7.580 | 1.00 | 18.22 | C | C |
| ATOM | 8619 | CD | GLU I | 236 | 3.760 | −4.040 | 7.926 | 1.00 | 21.59 | C | C |
| ATOM | 8620 | OE1 | GLU I | 236 | 4.381 | −4.645 | 7.027 | 1.00 | 24.41 | C | O |
| ATOM | 8621 | OE2 | GLU I | 236 | 4.182 | −3.944 | 9.097 | 1.00 | 21.59 | C | O |
| ATOM | 8622 | C | GLU I | 236 | −0.735 | −2.999 | 8.400 | 1.00 | 14.61 | C | C |
| ATOM | 8623 | O | GLU I | 236 | −1.191 | −3.334 | 7.307 | 1.00 | 14.82 | C | O |
| ATOM | 8624 | N | PHE I | 237 | −1.184 | −3.485 | 9.552 | 1.00 | 14.05 | C | N |
| ATOM | 8625 | CA | PHE I | 237 | −2.318 | −4.397 | 9.602 | 1.00 | 13.84 | C | C |
| ATOM | 8626 | CB | PHE I | 237 | −2.466 | −4.996 | 11.003 | 1.00 | 14.04 | C | C |
| ATOM | 8627 | CG | PHE I | 237 | −1.643 | −6.235 | 11.225 | 1.00 | 14.84 | C | C |
| ATOM | 8628 | CD1 | PHE I | 237 | −0.734 | −6.296 | 12.267 | 1.00 | 15.30 | C | C |
| ATOM | 8629 | CE1 | PHE I | 237 | −0.006 | −7.446 | 12.503 | 1.00 | 15.15 | C | C |
| ATOM | 8630 | CZ | PHE I | 237 | −0.197 | −8.557 | 11.708 | 1.00 | 15.19 | C | C |
| ATOM | 8631 | CE2 | PHE I | 237 | −1.110 | −8.514 | 10.673 | 1.00 | 15.02 | C | C |
| ATOM | 8632 | CD2 | PHE I | 237 | −1.831 | −7.360 | 10.439 | 1.00 | 15.03 | C | C |
| ATOM | 8633 | C | PHE I | 237 | −3.607 | −3.689 | 9.195 | 1.00 | 13.51 | C | C |
| ATOM | 8634 | O | PHE I | 237 | −4.387 | −4.213 | 8.399 | 1.00 | 13.43 | C | O |
| ATOM | 8635 | N | SER I | 238 | −3.830 | −2.503 | 9.754 | 1.00 | 13.11 | C | N |
| ATOM | 8636 | CA | SER I | 238 | −5.032 | −1.729 | 9.460 | 1.00 | 12.82 | C | C |
| ATOM | 8637 | CB | SER I | 238 | −5.049 | −0.438 | 10.279 | 1.00 | 12.86 | C | C |
| ATOM | 8638 | OG | SER I | 238 | −5.205 | −0.711 | 11.660 | 1.00 | 12.76 | C | O |
| ATOM | 8639 | C | SER I | 238 | −5.104 | −1.400 | 7.977 | 1.00 | 12.69 | C | C |
| ATOM | 8640 | O | SER I | 238 | −6.187 | −1.237 | 7.414 | 1.00 | 12.63 | C | O |
| ATOM | 8641 | N | VAL I | 239 | −3.937 | −1.313 | 7.349 | 1.00 | 12.59 | C | N |
| ATOM | 8642 | CA | VAL I | 239 | −3.858 | −1.042 | 5.923 | 1.00 | 12.80 | C | C |
| ATOM | 8643 | CB | VAL I | 239 | −2.482 | −0.473 | 5.524 | 1.00 | 12.71 | C | C |
| ATOM | 8644 | CG1 | VAL I | 239 | −2.390 | −0.321 | 4.012 | 1.00 | 13.63 | C | C |
| ATOM | 8645 | CG2 | VAL I | 239 | −2.239 | 0.859 | 6.214 | 1.00 | 13.57 | C | C |
| ATOM | 8646 | C | VAL I | 239 | −4.144 | −2.286 | 5.090 | 1.00 | 12.94 | C | C |
| ATOM | 8647 | O | VAL I | 239 | −4.320 | −2.190 | 3.877 | 1.00 | 13.10 | C | O |
| ATOM | 8648 | N | ASN I | 240 | −4.139 | −3.458 | 5.721 | 1.00 | 4.15 | C | N |
| ATOM | 8649 | CA | ASN I | 240 | −4.166 | −4.709 | 4.966 | 1.00 | 4.33 | C | C |
| ATOM | 8650 | CB | ASN I | 240 | −2.941 | −5.569 | 5.281 | 1.00 | 4.59 | C | C |
| ATOM | 8651 | CG | ASN I | 240 | −1.674 | −5.027 | 4.647 | 1.00 | 5.36 | C | C |
| ATOM | 8652 | OD1 | ASN I | 240 | −1.524 | −5.043 | 3.423 | 1.00 | 6.27 | C | O |
| ATOM | 8653 | ND2 | ASN I | 240 | −0.816 | −4.427 | 5.465 | 1.00 | 7.15 | C | N |
| ATOM | 8654 | C | ASN I | 240 | −5.454 | −5.529 | 5.074 | 1.00 | 4.19 | C | C |
| ATOM | 8655 | O | ASN I | 240 | −5.634 | −6.507 | 4.347 | 1.00 | 4.19 | C | O |
| ATOM | 8656 | N | ALA I | 241 | −6.385 | −5.073 | 5.904 | 1.00 | 3.93 | C | N |
| ATOM | 8657 | CA | ALA I | 241 | −7.593 | −5.840 | 6.186 | 1.00 | 3.90 | C | C |
| ATOM | 8658 | CB | ALA I | 241 | −8.563 | −5.749 | 5.020 | 1.00 | 3.86 | C | C |
| ATOM | 8659 | C | ALA I | 241 | −7.253 | −7.297 | 6.491 | 1.00 | 4.22 | C | C |
| ATOM | 8660 | O | ALA I | 241 | −7.778 | −8.212 | 5.856 | 1.00 | 4.47 | C | O |
| ATOM | 8661 | N | GLY I | 242 | −6.289 | −7.497 | 7.386 | 1.00 | 4.53 | C | N |
| ATOM | 8662 | CA | GLY I | 242 | −6.124 | −8.774 | 8.078 | 1.00 | 4.65 | C | C |
| ATOM | 8663 | C | GLY I | 242 | −5.398 | −9.848 | 7.282 | 1.00 | 4.67 | C | C |
| ATOM | 8664 | O | GLY I | 242 | −5.496 | −11.034 | 7.600 | 1.00 | 5.27 | C | O |
| ATOM | 8665 | N | VAL I | 243 | −4.611 | −9.433 | 6.292 | 1.00 | 4.37 | C | N |
| ATOM | 8666 | CA | VAL I | 243 | −3.780 | −10.366 | 5.531 | 1.00 | 4.32 | C | C |
| ATOM | 8667 | CB | VAL I | 243 | −4.570 | −11.042 | 4.389 | 1.00 | 3.99 | C | C |
| ATOM | 8668 | CG1 | VAL I | 243 | −3.987 | −12.411 | 4.078 | 1.00 | 4.22 | C | C |
| ATOM | 8669 | CG2 | VAL I | 243 | −6.042 | −11.154 | 4.748 | 1.00 | 3.85 | C | C |
| ATOM | 8670 | C | VAL I | 243 | −2.571 | −9.658 | 4.932 | 1.00 | 4.61 | C | C |
| ATOM | 8671 | O | VAL I | 243 | −2.704 | −8.607 | 4.308 | 1.00 | 4.89 | C | O |
| ATOM | 8672 | N | THR I | 244 | −1.402 | −10.269 | 5.072 | 1.00 | 4.95 | C | N |
| ATOM | 8673 | CA | THR I | 244 | −0.168 | −9.654 | 4.604 | 1.00 | 5.71 | C | C |
| ATOM | 8674 | CB | THR I | 244 | 0.678 | −9.128 | 5.775 | 1.00 | 6.11 | C | C |
| ATOM | 8675 | OG1 | THR I | 244 | −0.078 | −9.209 | 6.989 | 1.00 | 7.17 | C | O |
| ATOM | 8676 | CG2 | THR I | 244 | 1.091 | −7.686 | 5.532 | 1.00 | 7.55 | C | C |
| ATOM | 8677 | C | THR I | 244 | 0.664 | −10.649 | 3.813 | 1.00 | 5.61 | C | C |
| ATOM | 8678 | O | THR I | 244 | 0.811 | −11.803 | 4.214 | 1.00 | 5.85 | C | O |
| ATOM | 8679 | N | THR I | 245 | 1.346 | −10.150 | 2.791 | 1.00 | 5.53 | C | N |
| ATOM | 8680 | CA | THR I | 245 | 2.556 | −10.791 | 2.308 | 1.00 | 5.81 | C | C |
| ATOM | 8681 | CB | THR I | 245 | 2.426 | −11.170 | 0.818 | 1.00 | 6.13 | C | C |
| ATOM | 8682 | OG1 | THR I | 245 | 1.421 | −12.181 | 0.668 | 1.00 | 8.11 | C | O |
| ATOM | 8683 | CG2 | THR I | 245 | 3.746 | −11.696 | 0.281 | 1.00 | 6.63 | C | C |
| ATOM | 8684 | C | THR I | 245 | 3.748 | −9.865 | 2.542 | 1.00 | 5.73 | C | C |
| ATOM | 8685 | O | THR I | 245 | 3.568 | −8.705 | 2.916 | 1.00 | 5.86 | C | O |
| ATOM | 8686 | N | PRO I | 246 | 4.963 | −10.337 | 2.223 | 1.00 | 5.68 | C | N |
| ATOM | 8687 | CA | PRO I | 246 | 5.942 | −10.579 | 3.269 | 1.00 | 5.11 | C | C |
| ATOM | 8688 | CB | PRO I | 246 | 6.802 | −9.317 | 3.210 | 1.00 | 5.19 | C | C |
| ATOM | 8689 | CG | PRO I | 246 | 6.717 | −8.878 | 1.734 | 1.00 | 5.90 | C | C |
| ATOM | 8690 | CD | PRO I | 246 | 5.613 | −9.691 | 1.074 | 1.00 | 6.36 | C | C |
| ATOM | 8691 | C | PRO I | 246 | 5.360 | −10.786 | 4.664 | 1.00 | 4.85 | C | C |
| ATOM | 8692 | O | PRO I | 246 | 4.465 | −10.053 | 5.084 | 1.00 | 5.25 | C | O |
| ATOM | 8693 | N | VAL I | 247 | 5.795 | −11.859 | 5.316 | 1.00 | 4.35 | C | N |
| ATOM | 8694 | CA | VAL I | 247 | 5.555 | −12.048 | 6.739 | 1.00 | 3.82 | C | C |
| ATOM | 8695 | CB | VAL I | 247 | 5.603 | −13.536 | 7.127 | 1.00 | 3.52 | C | C |
| ATOM | 8696 | CG1 | VAL I | 247 | 5.206 | −13.717 | 8.584 | 1.00 | 3.62 | C | C |
| ATOM | 8697 | CG2 | VAL I | 247 | 4.694 | −14.347 | 6.218 | 1.00 | 3.60 | C | C |
| ATOM | 8698 | C | VAL I | 247 | 6.576 | −11.271 | 7.558 | 1.00 | 3.69 | C | C |
| ATOM | 8699 | O | VAL I | 247 | 7.744 | −11.653 | 7.641 | 1.00 | 3.36 | C | O |
| ATOM | 8700 | N | SER I | 248 | 6.172 | −10.077 | 7.973 | 1.00 | 3.72 | C | N |
| ATOM | 8701 | CA | SER I | 248 | 7.077 | −9.138 | 8.611 | 1.00 | 3.96 | C | C |
| ATOM | 8702 | CB | SER I | 248 | 6.291 | −7.973 | 9.214 | 1.00 | 4.07 | C | C |
| ATOM | 8703 | OG | SER I | 248 | 5.661 | −8.356 | 10.425 | 1.00 | 4.13 | C | O |
| ATOM | 8704 | C | SER I | 248 | 7.885 | −9.830 | 9.695 | 1.00 | 4.35 | C | C |
| ATOM | 8705 | O | SER I | 248 | 7.453 | −10.838 | 10.257 | 1.00 | 4.36 | C | O |
| ATOM | 8706 | N | THR I | 249 | 9.008 | −9.223 | 10.057 | 1.00 | 4.53 | C | N |
| ATOM | 8707 | CA | THR I | 249 | 9.752 | −9.638 | 11.235 | 1.00 | 4.61 | C | C |
| ATOM | 8708 | CB | THR I | 249 | 11.104 | −8.916 | 11.329 | 1.00 | 4.70 | C | C |
| ATOM | 8709 | OG1 | THR I | 249 | 10.889 | −7.544 | 11.679 | 1.00 | 5.42 | C | O |
| ATOM | 8710 | CG2 | THR I | 249 | 11.833 | −8.984 | 9.999 | 1.00 | 4.88 | C | C |
| ATOM | 8711 | C | THR I | 249 | 8.955 | −9.383 | 12.509 | 1.00 | 4.47 | C | C |
| ATOM | 8712 | O | THR I | 249 | 9.321 | −9.861 | 13.584 | 1.00 | 4.74 | C | O |
| ATOM | 8713 | N | TYR I | 250 | 7.864 | −8.633 | 12.388 | 1.00 | 4.32 | C | N |
| ATOM | 8714 | CA | TYR I | 250 | 6.956 | −8.435 | 13.512 | 1.00 | 4.42 | C | C |
| ATOM | 8715 | CB | TYR I | 250 | 6.195 | −7.113 | 13.381 | 1.00 | 4.91 | C | C |
| ATOM | 8716 | CG | TYR I | 250 | 7.072 | −5.881 | 13.272 | 1.00 | 6.81 | C | C |
| ATOM | 8717 | CD1 | TYR I | 250 | 6.791 | −4.897 | 12.333 | 1.00 | 8.89 | C | C |
| ATOM | 8718 | CE1 | TYR I | 250 | 7.556 | −3.752 | 12.243 | 1.00 | 10.73 | C | C |
| ATOM | 8719 | CZ | TYR I | 250 | 8.587 | −3.549 | 13.137 | 1.00 | 12.28 | C | C |
| ATOM | 8720 | OH | TYR I | 250 | 9.370 | −2.421 | 13.032 | 1.00 | 12.24 | C | O |
| ATOM | 8721 | CE2 | TYR I | 250 | 8.851 | −4.483 | 14.121 | 1.00 | 12.15 | C | C |
| ATOM | 8722 | CD2 | TYR I | 250 | 8.067 | −5.621 | 14.209 | 1.00 | 9.81 | C | C |
| ATOM | 8723 | C | TYR I | 250 | 5.973 | −9.592 | 13.662 | 1.00 | 4.21 | C | C |
| ATOM | 8724 | O | TYR I | 250 | 5.525 | −9.893 | 14.768 | 1.00 | 4.20 | C | O |
| ATOM | 8725 | N | MET I | 251 | 5.574 | −10.183 | 12.541 | 1.00 | 4.14 | C | N |
| ATOM | 8726 | CA | MET I | 251 | 4.731 | −11.372 | 12.574 | 1.00 | 4.15 | C | C |
| ATOM | 8727 | CB | MET I | 251 | 4.160 | −11.662 | 11.187 | 1.00 | 4.14 | C | C |
| ATOM | 8728 | CG | MET I | 251 | 3.297 | −10.547 | 10.630 | 1.00 | 4.51 | C | C |
| ATOM | 8729 | SD | MET I | 251 | 1.731 | −10.386 | 11.505 | 1.00 | 3.81 | C | S |
| ATOM | 8730 | CE | MET I | 251 | 0.800 | −9.412 | 10.327 | 1.00 | 4.55 | C | C |
| ATOM | 8731 | C | MET I | 251 | 5.540 | −12.565 | 13.057 | 1.00 | 4.13 | C | C |
| ATOM | 8732 | O | MET I | 251 | 5.127 | −13.289 | 13.964 | 1.00 | 4.24 | C | O |
| ATOM | 8733 | N | LEU I | 252 | 6.728 | −12.718 | 12.487 | 1.00 | 4.23 | C | N |
| ATOM | 8734 | CA | LEU I | 252 | 7.586 | −13.848 | 12.792 | 1.00 | 4.22 | C | C |
| ATOM | 8735 | CB | LEU I | 252 | 7.586 | −14.839 | 11.631 | 1.00 | 4.14 | C | C |
| ATOM | 8736 | CG | LEU I | 252 | 6.703 | −16.071 | 11.816 | 1.00 | 4.05 | C | C |
| ATOM | 8737 | CD1 | LEU I | 252 | 7.171 | −17.204 | 10.918 | 1.00 | 5.22 | C | C |
| ATOM | 8738 | CD2 | LEU I | 252 | 6.692 | −16.508 | 13.273 | 1.00 | 6.20 | C | C |
| ATOM | 8739 | C | LEU I | 252 | 8.998 | −13.356 | 13.040 | 1.00 | 4.62 | C | C |
| ATOM | 8740 | O | LEU I | 252 | 9.599 | −12.712 | 12.182 | 1.00 | 4.97 | C | O |
| ATOM | 8741 | N | THR I | 253 | 9.531 | −13.670 | 14.212 | 1.00 | 4.90 | C | N |
| ATOM | 8742 | CA | THR I | 253 | 10.942 | −13.463 | 14.472 | 1.00 | 5.57 | C | C |
| ATOM | 8743 | CB | THR I | 253 | 11.265 | −13.708 | 15.946 | 1.00 | 5.55 | C | C |
| ATOM | 8744 | OG1 | THR I | 253 | 10.337 | −12.987 | 16.765 | 1.00 | 6.43 | C | O |
| ATOM | 8745 | CG2 | THR I | 253 | 12.673 | −13.251 | 16.259 | 1.00 | 6.28 | C | C |
| ATOM | 8746 | C | THR I | 253 | 11.753 | −14.424 | 13.622 | 1.00 | 6.03 | C | C |
| ATOM | 8747 | O | THR I | 253 | 11.315 | −15.541 | 13.349 | 1.00 | 6.68 | C | O |
| ATOM | 8748 | N | ASN I | 254 | 12.961 | −14.015 | 13.255 | 1.00 | 6.39 | C | N |
| ATOM | 8749 | CA | ASN I | 254 | 13.951 | −14.973 | 12.800 | 1.00 | 6.79 | C | C |
| ATOM | 8750 | CB | ASN I | 254 | 15.341 | −14.348 | 12.762 | 1.00 | 6.85 | C | C |
| ATOM | 8751 | CG | ASN I | 254 | 16.324 | −15.177 | 11.964 | 1.00 | 6.81 | C | C |
| ATOM | 8752 | OD1 | ASN I | 254 | 16.152 | −15.366 | 10.760 | 1.00 | 7.67 | C | O |
| ATOM | 8753 | ND2 | ASN I | 254 | 17.271 | −15.799 | 12.657 | 1.00 | 6.19 | C | N |
| ATOM | 8754 | C | ASN I | 254 | 13.964 | −16.211 | 13.686 | 1.00 | 7.08 | C | C |
| ATOM | 8755 | O | ASN I | 254 | 13.654 | −17.312 | 13.231 | 1.00 | 6.90 | C | O |
| ATOM | 8756 | N | SER I | 255 | 14.283 | −16.017 | 14.962 | 1.00 | 7.21 | C | N |
| ATOM | 8757 | CA | SER I | 255 | 14.292 | −17.112 | 15.929 | 1.00 | 7.37 | C | C |
| ATOM | 8758 | CB | SER I | 255 | 14.338 | −16.568 | 17.360 | 1.00 | 8.09 | C | C |
| ATOM | 8759 | OG | SER I | 255 | 14.953 | −15.293 | 17.407 | 1.00 | 10.47 | C | O |
| ATOM | 8760 | C | SER I | 255 | 13.078 | −18.024 | 15.750 | 1.00 | 6.82 | C | C |
| ATOM | 8761 | O | SER I | 255 | 13.205 | −19.249 | 15.768 | 1.00 | 6.99 | C | O |
| ATOM | 8762 | N | GLU I | 256 | 11.901 | −17.422 | 15.611 | 1.00 | 6.17 | C | N |
| ATOM | 8763 | CA | GLU I | 256 | 10.658 | −18.184 | 15.544 | 1.00 | 6.13 | C | C |
| ATOM | 8764 | CB | GLU I | 256 | 9.452 | −17.266 | 15.742 | 1.00 | 6.22 | C | C |
| ATOM | 8765 | CG | GLU I | 256 | 9.210 | −16.863 | 17.185 | 1.00 | 8.41 | C | C |
| ATOM | 8766 | CD | GLU I | 256 | 8.527 | −15.516 | 17.299 | 1.00 | 10.26 | C | C |
| ATOM | 8767 | OE1 | GLU I | 256 | 8.232 | −14.907 | 16.248 | 1.00 | 11.90 | C | O |
| ATOM | 8768 | OE2 | GLU I | 256 | 8.275 | −15.070 | 18.437 | 1.00 | 9.55 | C | O |
| ATOM | 8769 | C | GLU I | 256 | 10.535 | −18.895 | 14.209 | 1.00 | 5.76 | C | C |
| ATOM | 8770 | O | GLU I | 256 | 10.154 | −20.064 | 14.154 | 1.00 | 5.93 | C | O |
| ATOM | 8771 | N | LEU I | 257 | 10.669 | −18.124 | 13.134 | 1.00 | 5.67 | C | N |
| ATOM | 8772 | CA | LEU I | 257 | 10.638 | −18.679 | 11.787 | 1.00 | 5.37 | C | C |
| ATOM | 8773 | CB | LEU I | 257 | 11.010 | −17.613 | 10.753 | 1.00 | 5.03 | C | C |
| ATOM | 8774 | CG | LEU I | 257 | 11.316 | −18.130 | 9.344 | 1.00 | 3.65 | C | C |
| ATOM | 8775 | CD1 | LEU I | 257 | 10.067 | −18.702 | 8.689 | 1.00 | 2.00 | C | C |
| ATOM | 8776 | CD2 | LEU I | 257 | 11.917 | −17.030 | 8.485 | 1.00 | 2.14 | C | C |
| ATOM | 8777 | C | LEU I | 257 | 11.580 | −19.871 | 11.670 | 1.00 | 5.64 | C | C |
| ATOM | 8778 | O | LEU I | 257 | 11.234 | −20.895 | 11.079 | 1.00 | 5.81 | C | O |
| ATOM | 8779 | N | LEU I | 258 | 12.764 | −19.741 | 12.259 | 1.00 | 5.75 | C | N |
| ATOM | 8780 | CA | LEU I | 258 | 13.808 | −20.739 | 12.081 | 1.00 | 5.91 | C | C |
| ATOM | 8781 | CB | LEU I | 258 | 15.156 | −20.215 | 12.583 | 1.00 | 6.10 | C | C |
| ATOM | 8782 | CG | LEU I | 258 | 16.059 | −19.626 | 11.495 | 1.00 | 5.75 | C | C |
| ATOM | 8783 | CD1 | LEU I | 258 | 17.208 | −18.836 | 12.101 | 1.00 | 6.33 | C | C |
| ATOM | 8784 | CD2 | LEU I | 258 | 16.577 | −20.724 | 10.577 | 1.00 | 7.33 | C | C |
| ATOM | 8785 | C | LEU I | 258 | 13.464 | −22.074 | 12.736 | 1.00 | 6.03 | C | C |
| ATOM | 8786 | O | LEU I | 258 | 13.589 | −23.125 | 12.109 | 1.00 | 5.73 | C | O |
| ATOM | 8787 | N | SER I | 259 | 12.980 | −22.030 | 13.974 | 1.00 | 6.47 | C | N |
| ATOM | 8788 | CA | SER I | 259 | 12.538 | −23.243 | 14.662 | 1.00 | 6.95 | C | C |
| ATOM | 8789 | CB | SER I | 259 | 12.400 | −23.000 | 16.168 | 1.00 | 7.05 | C | C |
| ATOM | 8790 | OG | SER I | 259 | 11.147 | −23.460 | 16.650 | 1.00 | 6.86 | C | O |
| ATOM | 8791 | C | SER I | 259 | 11.220 | −23.759 | 14.097 | 1.00 | 6.74 | C | C |
| ATOM | 8792 | O | SER I | 259 | 10.905 | −24.943 | 14.213 | 1.00 | 6.86 | C | O |
| ATOM | 8793 | N | LEU I | 260 | 10.411 | −22.844 | 13.576 | 1.00 | 6.31 | C | N |
| ATOM | 8794 | CA | LEU I | 260 | 9.188 | −23.211 | 12.880 | 1.00 | 6.09 | C | C |
| ATOM | 8795 | CB | LEU I | 260 | 8.407 | −21.957 | 12.484 | 1.00 | 6.09 | C | C |
| ATOM | 8796 | CG | LEU I | 260 | 7.140 | −21.665 | 13.291 | 1.00 | 6.24 | C | C |
| ATOM | 8797 | CD1 | LEU I | 260 | 6.291 | −20.595 | 12.611 | 1.00 | 6.53 | C | C |
| ATOM | 8798 | CD2 | LEU I | 260 | 6.337 | −22.944 | 13.521 | 1.00 | 5.98 | C | C |
| ATOM | 8799 | C | LEU I | 260 | 9.497 | −24.047 | 11.644 | 1.00 | 6.25 | C | C |
| ATOM | 8800 | O | LEU I | 260 | 8.809 | −25.028 | 11.361 | 1.00 | 6.37 | C | O |
| ATOM | 8801 | N | ILE I | 261 | 10.452 | −23.580 | 10.847 | 1.00 | 6.63 | C | N |
| ATOM | 8802 | CA | ILE I | 261 | 10.952 | −24.360 | 9.723 | 1.00 | 7.19 | C | C |
| ATOM | 8803 | CB | ILE I | 261 | 12.165 | −23.681 | 9.061 | 1.00 | 7.09 | C | C |
| ATOM | 8804 | CG1 | ILE I | 261 | 11.703 | −22.635 | 8.046 | 1.00 | 7.46 | C | C |
| ATOM | 8805 | CD1 | ILE I | 261 | 12.646 | −21.460 | 7.909 | 1.00 | 8.38 | C | C |
| ATOM | 8806 | CG2 | ILE I | 261 | 13.055 | −24.718 | 8.391 | 1.00 | 6.61 | C | C |
| ATOM | 8807 | C | ILE I | 261 | 11.389 | −25.723 | 10.228 | 1.00 | 8.01 | C | C |
| ATOM | 8808 | O | ILE I | 261 | 10.967 | −26.758 | 9.713 | 1.00 | 8.26 | C | O |
| ATOM | 8809 | N | ASN I | 262 | 12.206 | −25.708 | 11.273 | 1.00 | 9.05 | C | N |
| ATOM | 8810 | CA | ASN I | 262 | 12.640 | −26.935 | 11.912 | 1.00 | 9.88 | C | C |
| ATOM | 8811 | CB | ASN I | 262 | 13.220 | −26.636 | 13.292 | 1.00 | 9.99 | C | C |
| ATOM | 8812 | CG | ASN I | 262 | 14.327 | −27.590 | 13.672 | 1.00 | 11.16 | C | C |
| ATOM | 8813 | OD1 | ASN I | 262 | 14.709 | −28.457 | 12.886 | 1.00 | 12.70 | C | O |
| ATOM | 8814 | ND2 | ASN I | 262 | 14.912 | −27.379 | 14.844 | 1.00 | 13.20 | C | N |
| ATOM | 8815 | C | ASN I | 262 | 11.500 | −27.939 | 12.023 | 1.00 | 10.17 | C | C |
| ATOM | 8816 | O | ASN I | 262 | 11.661 | −29.108 | 11.675 | 1.00 | 10.52 | C | O |
| ATOM | 8817 | N | ASP I | 263 | 10.315 | −27.444 | 12.368 | 1.00 | 10.62 | C | N |
| ATOM | 8818 | CA | ASP I | 263 | 9.203 | −28.311 | 12.741 | 1.00 | 11.02 | C | C |
| ATOM | 8819 | CB | ASP I | 263 | 8.230 | −27.584 | 13.675 | 1.00 | 11.49 | C | C |
| ATOM | 8820 | CG | ASP I | 263 | 7.026 | −28.435 | 14.040 | 1.00 | 12.80 | C | C |
| ATOM | 8821 | OD1 | ASP I | 263 | 7.219 | −29.586 | 14.487 | 1.00 | 14.72 | C | O |
| ATOM | 8822 | OD2 | ASP I | 263 | 5.886 | −27.978 | 13.811 | 1.00 | 13.88 | C | O |
| ATOM | 8823 | C | ASP I | 263 | 8.460 | −28.855 | 11.520 | 1.00 | 10.92 | C | C |
| ATOM | 8824 | O | ASP I | 263 | 7.619 | −29.748 | 11.642 | 1.00 | 10.96 | C | O |
| ATOM | 8825 | N | MET I | 264 | 8.807 | −28.346 | 10.341 | 1.00 | 10.91 | C | N |
| ATOM | 8826 | CA | MET I | 264 | 8.105 | −28.707 | 9.110 | 1.00 | 10.78 | C | C |
| ATOM | 8827 | CB | MET I | 264 | 8.397 | −27.687 | 8.007 | 1.00 | 10.51 | C | C |
| ATOM | 8828 | CG | MET I | 264 | 7.934 | −26.276 | 8.329 | 1.00 | 11.26 | C | C |
| ATOM | 8829 | SD | MET I | 264 | 8.142 | −25.144 | 6.942 | 1.00 | 12.70 | C | S |
| ATOM | 8830 | CE | MET I | 264 | 9.799 | −25.565 | 6.411 | 1.00 | 13.92 | C | C |
| ATOM | 8831 | C | MET I | 264 | 8.466 | −30.116 | 8.631 | 1.00 | 10.85 | C | C |
| ATOM | 8832 | O | MET I | 264 | 9.644 | −30.472 | 8.578 | 1.00 | 11.03 | C | O |
| ATOM | 8833 | N | PRO I | 265 | 7.451 | −30.900 | 8.230 | 1.00 | 10.88 | C | N |
| ATOM | 8834 | CA | PRO I | 265 | 7.647 | −32.293 | 7.841 | 1.00 | 11.09 | C | C |
| ATOM | 8835 | CB | PRO I | 265 | 6.215 | −32.820 | 7.685 | 1.00 | 11.28 | C | C |
| ATOM | 8836 | CG | PRO I | 265 | 5.387 | −31.612 | 7.438 | 1.00 | 11.24 | C | C |
| ATOM | 8837 | CD | PRO I | 265 | 6.025 | −30.534 | 8.255 | 1.00 | 11.03 | C | C |
| ATOM | 8838 | C | PRO I | 265 | 8.400 | −32.420 | 6.521 | 1.00 | 11.19 | C | C |
| ATOM | 8839 | O | PRO I | 265 | 7.889 | −33.027 | 5.580 | 1.00 | 11.47 | C | O |
| ATOM | 8840 | N | ILE I | 266 | 9.642 | −31.940 | 6.488 | 1.00 | 11.54 | C | N |
| ATOM | 8841 | CA | ILE I | 266 | 10.448 | −31.980 | 5.266 | 1.00 | 12.15 | C | C |
| ATOM | 8842 | CB | ILE I | 266 | 10.586 | −30.587 | 4.621 | 1.00 | 12.22 | C | C |
| ATOM | 8843 | CG1 | ILE I | 266 | 11.051 | −29.559 | 5.654 | 1.00 | 12.53 | C | C |
| ATOM | 8844 | CD1 | ILE I | 266 | 11.557 | −28.269 | 5.047 | 1.00 | 12.40 | C | C |
| ATOM | 8845 | CG2 | ILE I | 266 | 9.271 | −30.157 | 3.984 | 1.00 | 12.74 | C | C |
| ATOM | 8846 | C | ILE I | 266 | 11.839 | −32.574 | 5.485 | 1.00 | 12.29 | C | C |
| ATOM | 8847 | O | ILE I | 266 | 12.291 | −32.724 | 6.619 | 1.00 | 12.36 | C | O |
| ATOM | 8848 | N | THR I | 267 | 12.498 | −32.936 | 4.388 | 1.00 | 12.29 | C | N |
| ATOM | 8849 | CA | THR I | 267 | 13.868 | −33.435 | 4.440 | 1.00 | 12.43 | C | C |
| ATOM | 8850 | CB | THR I | 267 | 14.386 | −33.809 | 3.039 | 1.00 | 12.40 | C | C |
| ATOM | 8851 | OG1 | THR I | 267 | 15.460 | −32.934 | 2.674 | 1.00 | 13.21 | C | O |
| ATOM | 8852 | CG2 | THR I | 267 | 13.274 | −33.699 | 2.010 | 1.00 | 12.21 | C | C |
| ATOM | 8853 | C | THR I | 267 | 14.797 | −32.391 | 5.046 | 1.00 | 12.37 | C | C |
| ATOM | 8854 | O | THR I | 267 | 14.403 | −31.244 | 5.251 | 1.00 | 12.29 | C | O |
| ATOM | 8855 | N | ASN I | 268 | 16.045 | −32.779 | 5.282 | 1.00 | 12.47 | C | N |
| ATOM | 8856 | CA | ASN I | 268 | 16.965 | −31.945 | 6.045 | 1.00 | 12.57 | C | C |
| ATOM | 8857 | CB | ASN I | 268 | 18.041 | −32.800 | 6.715 | 1.00 | 12.69 | C | C |
| ATOM | 8858 | CG | ASN I | 268 | 17.682 | −33.171 | 8.138 | 1.00 | 13.65 | C | C |
| ATOM | 8859 | OD1 | ASN I | 268 | 16.611 | −32.820 | 8.631 | 1.00 | 14.37 | C | O |
| ATOM | 8860 | ND2 | ASN I | 268 | 18.569 | −33.905 | 8.800 | 1.00 | 15.74 | C | N |
| ATOM | 8861 | C | ASN I | 268 | 17.608 | −30.843 | 5.211 | 1.00 | 12.43 | C | C |
| ATOM | 8862 | O | ASN I | 268 | 17.775 | −29.717 | 5.680 | 1.00 | 12.51 | C | O |
| ATOM | 8863 | N | ASP I | 269 | 18.038 | −31.194 | 4.005 | 1.00 | 12.34 | C | N |
| ATOM | 8864 | CA | ASP I | 269 | 18.574 | −30.211 | 3.072 | 1.00 | 12.33 | C | C |
| ATOM | 8865 | CB | ASP I | 269 | 19.015 | −30.891 | 1.777 | 1.00 | 12.89 | C | C |
| ATOM | 8866 | CG | ASP I | 269 | 20.071 | −31.950 | 2.007 | 1.00 | 14.84 | C | C |
| ATOM | 8867 | OD1 | ASP I | 269 | 21.002 | −31.699 | 2.800 | 1.00 | 16.90 | C | O |
| ATOM | 8868 | OD2 | ASP I | 269 | 19.952 | −33.047 | 1.422 | 1.00 | 17.65 | C | O |
| ATOM | 8869 | C | ASP I | 269 | 17.539 | −29.138 | 2.769 | 1.00 | 11.60 | C | C |
| ATOM | 8870 | O | ASP I | 269 | 17.861 | −27.952 | 2.696 | 1.00 | 11.65 | C | O |
| ATOM | 8871 | N | GLN I | 270 | 16.295 | −29.564 | 2.585 | 1.00 | 10.62 | C | N |
| ATOM | 8872 | CA | GLN I | 270 | 15.182 | −28.633 | 2.501 | 1.00 | 10.01 | C | C |
| ATOM | 8873 | CB | GLN I | 270 | 13.853 | −29.384 | 2.525 | 1.00 | 10.18 | C | C |
| ATOM | 8874 | CG | GLN I | 270 | 12.845 | −28.862 | 1.525 | 1.00 | 11.49 | C | C |
| ATOM | 8875 | CD | GLN I | 270 | 12.012 | −29.968 | 0.918 | 1.00 | 14.38 | C | C |
| ATOM | 8876 | OE1 | GLN I | 270 | 10.789 | −29.863 | 0.836 | 1.00 | 16.38 | C | O |
| ATOM | 8877 | NE2 | GLN I | 270 | 12.665 | −31.057 | 0.530 | 1.00 | 15.41 | C | N |
| ATOM | 8878 | C | GLN I | 270 | 15.228 | −27.620 | 3.636 | 1.00 | 9.48 | C | C |
| ATOM | 8879 | O | GLN I | 270 | 15.310 | −26.415 | 3.400 | 1.00 | 9.12 | C | O |
| ATOM | 8880 | N | LYS I | 271 | 15.170 | −28.112 | 4.868 | 1.00 | 9.19 | C | N |
| ATOM | 8881 | CA | LYS I | 271 | 15.211 | −27.240 | 6.035 | 1.00 | 8.89 | C | C |
| ATOM | 8882 | CB | LYS I | 271 | 15.253 | −28.064 | 7.322 | 1.00 | 8.52 | C | C |
| ATOM | 8883 | CG | LYS I | 271 | 13.948 | −28.773 | 7.643 | 1.00 | 9.14 | C | C |
| ATOM | 8884 | CD | LYS I | 271 | 13.927 | −29.280 | 9.076 | 1.00 | 10.72 | C | C |
| ATOM | 8885 | CE | LYS I | 271 | 13.274 | −30.648 | 9.163 | 1.00 | 10.79 | C | C |
| ATOM | 8886 | NZ | LYS I | 271 | 12.712 | −30.914 | 10.514 | 1.00 | 9.77 | C | N |
| ATOM | 8887 | C | LYS I | 271 | 16.405 | −26.295 | 5.973 | 1.00 | 8.83 | C | C |
| ATOM | 8888 | O | LYS I | 271 | 16.257 | −25.082 | 6.127 | 1.00 | 9.05 | C | O |
| ATOM | 8889 | N | LYS I | 272 | 17.583 | −26.854 | 5.707 | 1.00 | 8.65 | C | N |
| ATOM | 8890 | CA | LYS I | 272 | 18.796 | −26.058 | 5.548 | 1.00 | 8.50 | C | C |
| ATOM | 8891 | CB | LYS I | 272 | 19.977 | −26.948 | 5.145 | 1.00 | 9.14 | C | C |
| ATOM | 8892 | CG | LYS I | 272 | 21.158 | −26.189 | 4.547 | 1.00 | 11.58 | C | C |
| ATOM | 8893 | CD | LYS I | 272 | 22.213 | −27.138 | 3.991 | 1.00 | 15.78 | C | C |
| ATOM | 8894 | CE | LYS I | 272 | 23.576 | −26.464 | 3.891 | 1.00 | 17.65 | C | C |
| ATOM | 8895 | NZ | LYS I | 272 | 24.667 | −27.446 | 3.641 | 1.00 | 19.68 | C | N |
| ATOM | 8896 | C | LYS I | 272 | 18.599 | −24.956 | 4.514 | 1.00 | 7.88 | C | C |
| ATOM | 8897 | O | LYS I | 272 | 18.919 | −23.794 | 4.761 | 1.00 | 8.12 | C | O |
| ATOM | 8898 | N | LEU I | 273 | 18.111 | −25.336 | 3.338 | 1.00 | 6.95 | C | N |
| ATOM | 8899 | CA | LEU I | 273 | 17.867 | −24.375 | 2.271 | 1.00 | 5.70 | C | C |
| ATOM | 8900 | CB | LEU I | 273 | 17.235 | −25.063 | 1.060 | 1.00 | 5.85 | C | C |
| ATOM | 8901 | CG | LEU I | 273 | 16.754 | −24.131 | −0.054 | 1.00 | 4.65 | C | C |
| ATOM | 8902 | CD1 | LEU I | 273 | 17.928 | −23.571 | −0.841 | 1.00 | 4.57 | C | C |
| ATOM | 8903 | CD2 | LEU I | 273 | 15.777 | −24.846 | −0.972 | 1.00 | 3.69 | C | C |
| ATOM | 8904 | C | LEU I | 273 | 16.969 | −23.244 | 2.755 | 1.00 | 5.14 | C | C |
| ATOM | 8905 | O | LEU I | 273 | 17.345 | −22.074 | 2.702 | 1.00 | 5.25 | C | O |
| ATOM | 8906 | N | MET I | 274 | 15.776 | −23.601 | 3.221 | 1.00 | 4.52 | C | N |
| ATOM | 8907 | CA | MET I | 274 | 14.828 | −22.616 | 3.723 | 1.00 | 4.59 | C | C |
| ATOM | 8908 | CB | MET I | 274 | 13.599 | −23.308 | 4.314 | 1.00 | 4.35 | C | C |
| ATOM | 8909 | CG | MET I | 274 | 12.709 | −23.975 | 3.279 | 1.00 | 4.50 | C | C |
| ATOM | 8910 | SD | MET I | 274 | 11.194 | −24.645 | 3.989 | 1.00 | 5.51 | C | S |
| ATOM | 8911 | CE | MET I | 274 | 10.326 | −23.138 | 4.417 | 1.00 | 5.26 | C | C |
| ATOM | 8912 | C | MET I | 274 | 15.479 | −21.720 | 4.768 | 1.00 | 5.18 | C | C |
| ATOM | 8913 | O | MET I | 274 | 15.279 | −20.505 | 4.771 | 1.00 | 5.61 | C | O |
| ATOM | 8914 | N | SER I | 275 | 16.315 | −22.319 | 5.610 | 1.00 | 5.89 | C | N |
| ATOM | 8915 | CA | SER I | 275 | 16.961 | −21.588 | 6.688 | 1.00 | 6.18 | C | C |
| ATOM | 8916 | CB | SER I | 275 | 17.607 | −22.555 | 7.682 | 1.00 | 6.33 | C | C |
| ATOM | 8917 | OG | SER I | 275 | 16.654 | −23.470 | 8.193 | 1.00 | 5.91 | C | O |
| ATOM | 8918 | C | SER I | 275 | 17.996 | −20.603 | 6.157 | 1.00 | 6.58 | C | C |
| ATOM | 8919 | O | SER I | 275 | 18.181 | −19.526 | 6.721 | 1.00 | 7.03 | C | O |
| ATOM | 8920 | N | ASN I | 276 | 18.615 | −20.940 | 5.030 | 1.00 | 7.07 | C | N |
| ATOM | 8921 | CA | ASN I | 276 | 19.593 | −20.050 | 4.412 | 1.00 | 7.86 | C | C |
| ATOM | 8922 | CB | ASN I | 276 | 20.696 | −20.854 | 3.711 | 1.00 | 8.34 | C | C |
| ATOM | 8923 | CG | ASN I | 276 | 21.868 | −21.177 | 4.633 | 1.00 | 9.77 | C | C |
| ATOM | 8924 | OD1 | ASN I | 276 | 22.167 | −20.430 | 5.567 | 1.00 | 11.84 | C | O |
| ATOM | 8925 | ND2 | ASN I | 276 | 22.557 | −22.278 | 4.349 | 1.00 | 11.15 | C | N |
| ATOM | 8926 | C | ASN I | 276 | 18.976 | −19.029 | 3.453 | 1.00 | 7.71 | C | C |
| ATOM | 8927 | O | ASN I | 276 | 19.695 | −18.349 | 2.722 | 1.00 | 7.84 | C | O |
| ATOM | 8928 | N | ASN I | 277 | 17.655 | −18.870 | 3.509 | 1.00 | 7.77 | C | N |
| ATOM | 8929 | CA | ASN I | 277 | 16.938 | −18.098 | 2.491 | 1.00 | 7.85 | C | C |
| ATOM | 8930 | CB | ASN I | 277 | 16.575 | −18.987 | 1.304 | 1.00 | 7.58 | C | C |
| ATOM | 8931 | CG | ASN I | 277 | 17.725 | −19.157 | 0.335 | 1.00 | 8.29 | C | C |
| ATOM | 8932 | OD1 | ASN I | 277 | 18.053 | −18.240 | −0.417 | 1.00 | 9.04 | C | O |
| ATOM | 8933 | ND2 | ASN I | 277 | 18.427 | −20.279 | 0.443 | 1.00 | 9.57 | C | N |
| ATOM | 8934 | C | ASN I | 277 | 15.697 | −17.371 | 3.006 | 1.00 | 7.86 | C | C |
| ATOM | 8935 | O | ASN I | 277 | 14.826 | −16.977 | 2.229 | 1.00 | 7.85 | C | O |
| ATOM | 8936 | N | VAL I | 278 | 15.593 | −17.272 | 4.328 | 1.00 | 7.67 | C | N |
| ATOM | 8937 | CA | VAL I | 278 | 14.554 | −16.489 | 4.998 | 1.00 | 7.77 | C | C |
| ATOM | 8938 | CB | VAL I | 278 | 15.120 | −15.739 | 6.215 | 1.00 | 7.98 | C | C |
| ATOM | 8939 | CG1 | VAL I | 278 | 15.116 | −16.638 | 7.440 | 1.00 | 7.21 | C | C |
| ATOM | 8940 | CG2 | VAL I | 278 | 16.524 | −15.234 | 5.918 | 1.00 | 8.99 | C | C |
| ATOM | 8941 | C | VAL I | 278 | 13.813 | −15.497 | 4.105 | 1.00 | 7.76 | C | C |
| ATOM | 8942 | O | VAL I | 278 | 12.583 | −15.441 | 4.123 | 1.00 | 7.84 | C | O |
| ATOM | 8943 | N | GLN I | 279 | 14.562 | −14.581 | 3.501 | 1.00 | 7.47 | C | N |
| ATOM | 8944 | CA | GLN I | 279 | 13.966 | −13.446 | 2.809 | 1.00 | 7.07 | C | C |
| ATOM | 8945 | CB | GLN I | 279 | 15.038 | −12.668 | 2.045 | 1.00 | 7.62 | C | C |
| ATOM | 8946 | CG | GLN I | 279 | 14.870 | −11.159 | 2.116 | 1.00 | 10.35 | C | C |
| ATOM | 8947 | CD | GLN I | 279 | 15.712 | −10.430 | 1.091 | 1.00 | 13.70 | C | C |
| ATOM | 8948 | OE1 | GLN I | 279 | 16.199 | −11.027 | 0.132 | 1.00 | 15.06 | C | O |
| ATOM | 8949 | NE2 | GLN I | 279 | 15.865 | −9.124 | 1.275 | 1.00 | 13.97 | C | N |
| ATOM | 8950 | C | GLN I | 279 | 12.868 | −13.901 | 1.851 | 1.00 | 6.02 | C | C |
| ATOM | 8951 | O | GLN I | 279 | 11.734 | −13.423 | 1.912 | 1.00 | 5.45 | C | O |
| ATOM | 8952 | N | ILE I | 280 | 13.249 | −14.748 | 0.901 | 1.00 | 5.25 | C | N |
| ATOM | 8953 | CA | ILE I | 280 | 12.304 | −15.343 | −0.031 | 1.00 | 4.74 | C | C |
| ATOM | 8954 | CB | ILE I | 280 | 12.978 | −16.451 | −0.860 | 1.00 | 4.58 | C | C |
| ATOM | 8955 | CG1 | ILE I | 280 | 14.333 | −15.973 | −1.385 | 1.00 | 4.16 | C | C |
| ATOM | 8956 | CD1 | ILE I | 280 | 14.238 | −15.065 | −2.587 | 1.00 | 3.88 | C | C |
| ATOM | 8957 | CG2 | ILE I | 280 | 12.065 | −16.900 | −1.996 | 1.00 | 3.92 | C | C |
| ATOM | 8958 | C | ILE I | 280 | 11.122 | −15.946 | 0.716 | 1.00 | 4.43 | C | C |
| ATOM | 8959 | O | ILE I | 280 | 9.972 | −15.547 | 0.516 | 1.00 | 4.39 | C | O |
| ATOM | 8960 | N | VAL I | 281 | 11.409 | −16.970 | 1.513 | 1.00 | 4.05 | C | N |
| ATOM | 8961 | CA | VAL I | 281 | 10.390 | −17.623 | 2.319 | 1.00 | 3.58 | C | C |
| ATOM | 8962 | CB | VAL I | 281 | 11.010 | −18.335 | 3.534 | 1.00 | 3.13 | C | C |
| ATOM | 8963 | CG1 | VAL I | 281 | 10.006 | −19.291 | 4.158 | 1.00 | 2.34 | C | C |
| ATOM | 8964 | CG2 | VAL I | 281 | 12.277 | −19.073 | 3.127 | 1.00 | 2.21 | C | C |
| ATOM | 8965 | C | VAL I | 281 | 9.373 | −16.601 | 2.806 | 1.00 | 3.96 | C | C |
| ATOM | 8966 | O | VAL I | 281 | 8.186 | −16.694 | 2.495 | 1.00 | 4.15 | C | O |
| ATOM | 8967 | N | ARG I | 282 | 9.857 | −15.593 | 3.522 | 1.00 | 4.23 | C | N |
| ATOM | 8968 | CA | ARG I | 282 | 8.995 | −14.530 | 4.016 | 1.00 | 4.45 | C | C |
| ATOM | 8969 | CB | ARG I | 282 | 9.829 | −13.427 | 4.656 | 1.00 | 3.96 | C | C |
| ATOM | 8970 | CG | ARG I | 282 | 10.637 | −13.896 | 5.841 | 1.00 | 2.99 | C | C |
| ATOM | 8971 | CD | ARG I | 282 | 10.550 | −12.909 | 6.979 | 1.00 | 2.28 | C | C |
| ATOM | 8972 | NE | ARG I | 282 | 11.505 | −13.236 | 8.029 | 1.00 | 2.00 | C | N |
| ATOM | 8973 | CZ | ARG I | 282 | 11.175 | −13.463 | 9.297 | 1.00 | 2.44 | C | C |
| ATOM | 8974 | NH1 | ARG I | 282 | 9.914 | −13.339 | 9.690 | 1.00 | 2.04 | C | N |
| ATOM | 8975 | NH2 | ARG I | 282 | 12.099 | −13.847 | 10.167 | 1.00 | 3.44 | C | N |
| ATOM | 8976 | C | ARG I | 282 | 8.144 | −13.952 | 2.898 | 1.00 | 5.32 | C | C |
| ATOM | 8977 | O | ARG I | 282 | 6.924 | −13.854 | 3.022 | 1.00 | 5.95 | C | O |
| ATOM | 8978 | N | GLN I | 283 | 8.794 | −13.551 | 1.812 | 1.00 | 5.50 | C | N |
| ATOM | 8979 | CA | GLN I | 283 | 8.083 | −12.995 | 0.671 | 1.00 | 5.80 | C | C |
| ATOM | 8980 | CB | GLN I | 283 | 9.064 | −12.580 | −0.421 | 1.00 | 6.19 | C | C |
| ATOM | 8981 | CG | GLN I | 283 | 9.908 | −11.381 | −0.047 | 1.00 | 9.68 | C | C |
| ATOM | 8982 | CD | GLN I | 283 | 11.088 | −11.192 | −0.971 | 1.00 | 13.92 | C | C |
| ATOM | 8983 | OE1 | GLN I | 283 | 11.266 | −11.939 | −1.934 | 1.00 | 16.20 | C | O |
| ATOM | 8984 | NE2 | GLN I | 283 | 11.885 | −10.164 | −0.706 | 1.00 | 14.17 | C | N |
| ATOM | 8985 | C | GLN I | 283 | 7.071 | −13.988 | 0.120 | 1.00 | 5.44 | C | C |
| ATOM | 8986 | O | GLN I | 283 | 6.069 | −13.598 | −0.479 | 1.00 | 5.43 | C | O |
| ATOM | 8987 | N | GLN I | 284 | 7.302 | −15.269 | 0.387 | 1.00 | 5.26 | C | N |
| ATOM | 8988 | CA | GLN I | 284 | 6.465 | −16.321 | −0.172 | 1.00 | 5.18 | C | C |
| ATOM | 8989 | CB | GLN I | 284 | 7.303 | −17.549 | −0.517 | 1.00 | 5.32 | C | C |
| ATOM | 8990 | CG | GLN I | 284 | 7.689 | −17.619 | −1.981 | 1.00 | 6.85 | C | C |
| ATOM | 8991 | CD | GLN I | 284 | 8.548 | −18.821 | −2.304 | 1.00 | 9.21 | C | C |
| ATOM | 8992 | OE1 | GLN I | 284 | 9.398 | −19.227 | −1.511 | 1.00 | 9.74 | C | O |
| ATOM | 8993 | NE2 | GLN I | 284 | 8.332 | −19.400 | −3.477 | 1.00 | 9.79 | C | N |
| ATOM | 8994 | C | GLN I | 284 | 5.345 | −16.705 | 0.779 | 1.00 | 4.67 | C | C |
| ATOM | 8995 | O | GLN I | 284 | 4.485 | −17.520 | 0.447 | 1.00 | 4.19 | C | O |
| ATOM | 8996 | N | SER I | 285 | 5.367 | −16.116 | 1.968 | 1.00 | 4.51 | C | N |
| ATOM | 8997 | CA | SER I | 285 | 4.457 | −16.516 | 3.027 | 1.00 | 4.39 | C | C |
| ATOM | 8998 | CB | SER I | 285 | 5.218 | −16.729 | 4.336 | 1.00 | 4.13 | C | C |
| ATOM | 8999 | OG | SER I | 285 | 6.328 | −17.588 | 4.146 | 1.00 | 4.40 | C | O |
| ATOM | 9000 | C | SER I | 285 | 3.359 | −15.481 | 3.220 | 1.00 | 4.44 | C | C |
| ATOM | 9001 | O | SER I | 285 | 3.483 | −14.335 | 2.785 | 1.00 | 4.82 | C | O |
| ATOM | 9002 | N | TYR I | 286 | 2.268 | −15.908 | 3.842 | 1.00 | 4.53 | C | N |
| ATOM | 9003 | CA | TYR I | 286 | 1.194 | −15.002 | 4.213 | 1.00 | 4.80 | C | C |
| ATOM | 9004 | CB | TYR I | 286 | −0.116 | −15.441 | 3.559 | 1.00 | 4.88 | C | C |
| ATOM | 9005 | CG | TYR I | 286 | −0.135 | −15.272 | 2.059 | 1.00 | 5.68 | C | C |
| ATOM | 9006 | CD1 | TYR I | 286 | 0.713 | −16.013 | 1.245 | 1.00 | 7.34 | C | C |
| ATOM | 9007 | CE1 | TYR I | 286 | 0.695 | −15.864 | −0.126 | 1.00 | 9.02 | C | C |
| ATOM | 9008 | CZ | TYR I | 286 | −0.190 | −14.979 | −0.701 | 1.00 | 9.40 | C | C |
| ATOM | 9009 | OH | TYR I | 286 | −0.206 | −14.824 | −2.066 | 1.00 | 10.44 | C | O |
| ATOM | 9010 | CE2 | TYR I | 286 | −1.054 | −14.244 | 0.083 | 1.00 | 9.38 | C | C |
| ATOM | 9011 | CD2 | TYR I | 286 | −1.024 | −14.395 | 1.453 | 1.00 | 7.39 | C | C |
| ATOM | 9012 | C | TYR I | 286 | 1.037 | −14.978 | 5.726 | 1.00 | 4.76 | C | C |
| ATOM | 9013 | O | TYR I | 286 | 1.499 | −15.883 | 6.421 | 1.00 | 5.25 | C | O |
| ATOM | 9014 | N | SER I | 287 | 0.403 | −13.929 | 6.234 | 1.00 | 4.52 | C | N |
| ATOM | 9015 | CA | SER I | 287 | −0.072 | −13.924 | 7.609 | 1.00 | 4.45 | C | C |
| ATOM | 9016 | CB | SER I | 287 | 0.779 | −12.989 | 8.470 | 1.00 | 4.57 | C | C |
| ATOM | 9017 | OG | SER I | 287 | 0.113 | −12.662 | 9.678 | 1.00 | 4.38 | C | O |
| ATOM | 9018 | C | SER I | 287 | −1.535 | −13.516 | 7.675 | 1.00 | 4.61 | C | C |
| ATOM | 9019 | O | SER I | 287 | −1.920 | −12.460 | 7.172 | 1.00 | 4.40 | C | O |
| ATOM | 9020 | N | ILE I | 288 | −2.364 | −14.425 | 8.171 | 1.00 | 5.11 | C | N |
| ATOM | 9021 | CA | ILE I | 288 | −3.788 | −14.171 | 8.302 | 1.00 | 5.81 | C | C |
| ATOM | 9022 | CB | ILE I | 288 | −4.610 | −15.393 | 7.863 | 1.00 | 5.52 | C | C |
| ATOM | 9023 | CG1 | ILE I | 288 | −3.811 | −16.253 | 6.878 | 1.00 | 5.47 | C | C |
| ATOM | 9024 | CD1 | ILE I | 288 | −3.975 | −15.852 | 5.429 | 1.00 | 6.23 | C | C |
| ATOM | 9025 | CG2 | ILE I | 288 | −5.939 | −14.957 | 7.271 | 1.00 | 6.22 | C | C |
| ATOM | 9026 | C | ILE I | 288 | −4.123 | −13.848 | 9.750 | 1.00 | 6.71 | C | C |
| ATOM | 9027 | O | ILE I | 288 | −3.759 | −14.593 | 10.660 | 1.00 | 7.30 | C | O |
| ATOM | 9028 | N | MET I | 289 | −4.874 | −12.771 | 9.952 | 1.00 | 7.71 | C | N |
| ATOM | 9029 | CA | MET I | 289 | −5.451 | −12.476 | 11.257 | 1.00 | 8.57 | C | C |
| ATOM | 9030 | CB | MET I | 289 | −6.099 | −11.090 | 11.246 | 1.00 | 8.71 | C | C |
| ATOM | 9031 | CG | MET I | 289 | −6.131 | −10.412 | 12.603 | 1.00 | 9.70 | C | C |
| ATOM | 9032 | SD | MET I | 289 | −7.100 | −8.895 | 12.581 | 1.00 | 12.59 | C | S |
| ATOM | 9033 | CE | MET I | 289 | −8.569 | −9.452 | 11.721 | 1.00 | 12.41 | C | C |
| ATOM | 9034 | C | MET I | 289 | −6.477 | −13.538 | 11.658 | 1.00 | 9.12 | C | C |
| ATOM | 9035 | O | MET I | 289 | −7.322 | −13.925 | 10.851 | 1.00 | 9.31 | C | O |
| ATOM | 9036 | N | SER I | 290 | −6.406 | −13.998 | 12.907 | 1.00 | 9.80 | C | N |
| ATOM | 9037 | CA | SER I | 290 | −7.290 | −15.062 | 13.388 | 1.00 | 10.49 | C | C |
| ATOM | 9038 | CB | SER I | 290 | −6.494 | −16.195 | 14.035 | 1.00 | 10.64 | C | C |
| ATOM | 9039 | OG | SER I | 290 | −7.316 | −17.328 | 14.257 | 1.00 | 11.99 | C | O |
| ATOM | 9040 | C | SER I | 290 | −8.385 | −14.573 | 14.335 | 1.00 | 10.75 | C | C |
| ATOM | 9041 | O | SER I | 290 | −9.563 | −14.595 | 13.980 | 1.00 | 10.89 | C | O |
| ATOM | 9042 | N | ILE I | 291 | −8.018 | −14.243 | 15.571 | 1.00 | 11.04 | C | N |
| ATOM | 9043 | CA | ILE I | 291 | −8.955 | −13.562 | 16.461 | 1.00 | 11.41 | C | C |
| ATOM | 9044 | CB | ILE I | 291 | −9.796 | −14.539 | 17.305 | 1.00 | 11.80 | C | C |
| ATOM | 9045 | CG1 | ILE I | 291 | −9.096 | −15.893 | 17.419 | 1.00 | 11.81 | C | C |
| ATOM | 9046 | CD1 | ILE I | 291 | −8.227 | −16.028 | 18.647 | 1.00 | 11.76 | C | C |
| ATOM | 9047 | CG2 | ILE I | 291 | −11.179 | −14.707 | 16.700 | 1.00 | 12.39 | C | C |
| ATOM | 9048 | C | ILE I | 291 | −8.349 | −12.481 | 17.346 | 1.00 | 11.13 | C | C |
| ATOM | 9049 | O | ILE I | 291 | −7.316 | −12.681 | 17.986 | 1.00 | 10.55 | C | O |
| ATOM | 9050 | N | ILE I | 292 | −9.034 | −11.343 | 17.398 | 1.00 | 11.36 | C | N |
| ATOM | 9051 | CA | ILE I | 292 | −8.605 | −10.215 | 18.211 | 1.00 | 11.82 | C | C |
| ATOM | 9052 | CB | ILE I | 292 | −8.651 | −8.884 | 17.421 | 1.00 | 11.86 | C | C |
| ATOM | 9053 | CG1 | ILE I | 292 | −9.052 | −9.127 | 15.963 | 1.00 | 13.42 | C | C |
| ATOM | 9054 | CD1 | ILE I | 292 | −7.968 | −9.771 | 15.126 | 1.00 | 15.86 | C | C |
| ATOM | 9055 | CG2 | ILE I | 292 | −7.311 | −8.160 | 17.508 | 1.00 | 10.85 | C | C |
| ATOM | 9056 | C | ILE I | 292 | −9.486 | −10.092 | 19.444 | 1.00 | 11.72 | C | C |
| ATOM | 9057 | O | ILE I | 292 | −10.713 | −10.029 | 19.346 | 1.00 | 11.28 | C | O |
| ATOM | 9058 | N | LYS I | 293 | −8.857 | −10.180 | 20.607 | 1.00 | 12.11 | C | N |
| ATOM | 9059 | CA | LYS I | 293 | −9.479 | −9.728 | 21.834 | 1.00 | 12.92 | C | C |
| ATOM | 9060 | CB | LYS I | 293 | −9.599 | −10.889 | 22.820 | 1.00 | 13.15 | C | C |
| ATOM | 9061 | CG | LYS I | 293 | −10.034 | −12.194 | 22.174 | 1.00 | 14.17 | C | C |
| ATOM | 9062 | CD | LYS I | 293 | −10.958 | −12.974 | 23.091 | 1.00 | 15.37 | C | C |
| ATOM | 9063 | CE | LYS I | 293 | −11.869 | −13.899 | 22.305 | 1.00 | 16.68 | C | C |
| ATOM | 9064 | NZ | LYS I | 293 | −12.822 | −14.614 | 23.197 | 1.00 | 18.18 | C | N |
| ATOM | 9065 | C | LYS I | 293 | −8.670 | −8.584 | 22.433 | 1.00 | 13.32 | C | C |
| ATOM | 9066 | O | LYS I | 293 | −7.518 | −8.366 | 22.055 | 1.00 | 13.17 | C | O |
| ATOM | 9067 | N | GLU I | 294 | −9.337 | −7.759 | 23.233 | 1.00 | 14.07 | C | N |
| ATOM | 9068 | CA | GLU I | 294 | −8.714 | −6.572 | 23.805 | 1.00 | 15.07 | C | C |
| ATOM | 9069 | CB | GLU I | 294 | −9.608 | −5.975 | 24.896 | 1.00 | 15.94 | C | C |
| ATOM | 9070 | CG | GLU I | 294 | −10.872 | −5.307 | 24.373 | 1.00 | 19.87 | C | C |
| ATOM | 9071 | CD | GLU I | 294 | −11.569 | −4.471 | 25.427 | 1.00 | 23.88 | C | C |
| ATOM | 9072 | OE1 | GLU I | 294 | −10.994 | −3.449 | 25.854 | 1.00 | 23.75 | C | O |
| ATOM | 9073 | OE2 | GLU I | 294 | −12.723 | −4.799 | 25.777 | 1.00 | 25.81 | C | O |
| ATOM | 9074 | C | GLU I | 294 | −7.324 | −6.878 | 24.362 | 1.00 | 14.40 | C | C |
| ATOM | 9075 | O | GLU I | 294 | −6.433 | −6.031 | 24.319 | 1.00 | 14.33 | C | O |
| ATOM | 9076 | N | GLU I | 295 | −7.144 | −8.092 | 24.879 | 1.00 | 13.97 | C | N |
| ATOM | 9077 | CA | GLU I | 295 | −5.908 | −8.452 | 25.568 | 1.00 | 13.84 | C | C |
| ATOM | 9078 | CB | GLU I | 295 | −6.161 | −8.721 | 27.055 | 1.00 | 14.67 | C | C |
| ATOM | 9079 | CG | GLU I | 295 | −7.620 | −8.914 | 27.427 | 1.00 | 17.56 | C | C |
| ATOM | 9080 | CD | GLU I | 295 | −8.245 | −10.112 | 26.743 | 1.00 | 21.41 | C | C |
| ATOM | 9081 | OE1 | GLU I | 295 | −8.990 | −9.909 | 25.761 | 1.00 | 22.35 | C | O |
| ATOM | 9082 | OE2 | GLU I | 295 | −8.090 | −11.241 | 27.257 | 1.00 | 23.20 | C | O |
| ATOM | 9083 | C | GLU I | 295 | −5.166 | −9.630 | 24.933 | 1.00 | 12.77 | C | C |
| ATOM | 9084 | O | GLU I | 295 | −4.166 | −10.100 | 25.476 | 1.00 | 12.58 | C | O |
| ATOM | 9085 | N | VAL I | 296 | −5.701 | −10.162 | 23.838 | 1.00 | 11.72 | C | N |
| ATOM | 9086 | CA | VAL I | 296 | −4.970 | −11.150 | 23.051 | 1.00 | 10.65 | C | C |
| ATOM | 9087 | CB | VAL I | 296 | −5.245 | −12.589 | 23.517 | 1.00 | 10.47 | C | C |
| ATOM | 9088 | CG1 | VAL I | 296 | −4.277 | −13.546 | 22.846 | 1.00 | 9.77 | C | C |
| ATOM | 9089 | CG2 | VAL I | 296 | −5.118 | −12.688 | 25.026 | 1.00 | 11.31 | C | C |
| ATOM | 9090 | C | VAL I | 296 | −5.245 | −11.039 | 21.562 | 1.00 | 10.26 | C | C |
| ATOM | 9091 | O | VAL I | 296 | −6.395 | −10.940 | 21.133 | 1.00 | 10.09 | C | O |
| ATOM | 9092 | N | LEU I | 297 | −4.184 | −11.178 | 20.777 | 1.00 | 9.77 | C | N |
| ATOM | 9093 | CA | LEU I | 297 | −4.302 | −11.269 | 19.332 | 1.00 | 8.97 | C | C |
| ATOM | 9094 | CB | LEU I | 297 | −3.577 | −10.097 | 18.672 | 1.00 | 8.64 | C | C |
| ATOM | 9095 | CG | LEU I | 297 | −3.382 | −10.209 | 17.160 | 1.00 | 9.01 | C | C |
| ATOM | 9096 | CD1 | LEU I | 297 | −4.721 | −10.384 | 16.459 | 1.00 | 9.79 | C | C |
| ATOM | 9097 | CD2 | LEU I | 297 | −2.645 | −8.994 | 16.620 | 1.00 | 9.43 | C | C |
| ATOM | 9098 | C | LEU I | 297 | −3.725 | −12.587 | 18.829 | 1.00 | 8.34 | C | C |
| ATOM | 9099 | O | LEU I | 297 | −2.565 | −12.908 | 19.089 | 1.00 | 8.64 | C | O |
| ATOM | 9100 | N | ALA I | 298 | −4.545 | −13.352 | 18.116 | 1.00 | 7.21 | C | N |
| ATOM | 9101 | CA | ALA I | 298 | −4.095 | −14.600 | 17.515 | 1.00 | 6.33 | C | C |
| ATOM | 9102 | CB | ALA I | 298 | −4.908 | −15.770 | 18.047 | 1.00 | 6.68 | C | C |
| ATOM | 9103 | C | ALA I | 298 | −4.207 | −14.524 | 16.002 | 1.00 | 5.85 | C | C |
| ATOM | 9104 | O | ALA I | 298 | −5.179 | −13.991 | 15.468 | 1.00 | 5.96 | C | O |
| ATOM | 9105 | N | TYR I | 299 | −3.197 | −15.040 | 15.314 | 1.00 | 5.28 | C | N |
| ATOM | 9106 | CA | TYR I | 299 | −3.197 | −15.042 | 13.861 | 1.00 | 4.63 | C | C |
| ATOM | 9107 | CB | TYR I | 299 | −2.583 | −13.749 | 13.324 | 1.00 | 4.36 | C | C |
| ATOM | 9108 | CG | TYR I | 299 | −1.177 | −13.490 | 13.812 | 1.00 | 4.34 | C | C |
| ATOM | 9109 | CD1 | TYR I | 299 | −0.080 | −13.970 | 13.110 | 1.00 | 4.48 | C | C |
| ATOM | 9110 | CE1 | TYR I | 299 | 1.207 | −13.733 | 13.549 | 1.00 | 4.55 | C | C |
| ATOM | 9111 | CZ | TYR I | 299 | 1.409 | −13.011 | 14.705 | 1.00 | 3.72 | C | C |
| ATOM | 9112 | OH | TYR I | 299 | 2.690 | −12.775 | 15.149 | 1.00 | 2.38 | C | O |
| ATOM | 9113 | CE2 | TYR I | 299 | 0.336 | −12.526 | 15.422 | 1.00 | 4.13 | C | C |
| ATOM | 9114 | CD2 | TYR I | 299 | −0.947 | −12.767 | 14.975 | 1.00 | 4.38 | C | C |
| ATOM | 9115 | C | TYR I | 299 | −2.440 | −16.243 | 13.322 | 1.00 | 4.32 | C | C |
| ATOM | 9116 | O | TYR I | 299 | −1.602 | −16.822 | 14.012 | 1.00 | 4.67 | C | O |
| ATOM | 9117 | N | VAL I | 300 | −2.746 | −16.622 | 12.087 | 1.00 | 3.87 | C | N |
| ATOM | 9118 | CA | VAL I | 300 | −2.130 | −17.792 | 11.486 | 1.00 | 3.57 | C | C |
| ATOM | 9119 | CB | VAL I | 300 | −3.169 | −18.702 | 10.805 | 1.00 | 3.52 | C | C |
| ATOM | 9120 | CG1 | VAL I | 300 | −2.475 | −19.749 | 9.943 | 1.00 | 3.44 | C | C |
| ATOM | 9121 | CG2 | VAL I | 300 | −4.053 | −19.368 | 11.851 | 1.00 | 3.90 | C | C |
| ATOM | 9122 | C | VAL I | 300 | −1.060 | −17.392 | 10.482 | 1.00 | 3.53 | C | C |
| ATOM | 9123 | O | VAL I | 300 | −1.299 | −16.575 | 9.593 | 1.00 | 3.68 | C | O |
| ATOM | 9124 | N | VAL I | 301 | 0.148 | −17.899 | 10.695 | 1.00 | 3.41 | C | N |
| ATOM | 9125 | CA | VAL I | 301 | 1.193 | −17.816 | 9.689 | 1.00 | 3.36 | C | C |
| ATOM | 9126 | CB | VAL I | 301 | 2.593 | −17.825 | 10.324 | 1.00 | 3.24 | C | C |
| ATOM | 9127 | CG1 | VAL I | 301 | 3.655 | −18.061 | 9.261 | 1.00 | 3.76 | C | C |
| ATOM | 9128 | CG2 | VAL I | 301 | 2.849 | −16.519 | 11.059 | 1.00 | 3.10 | C | C |
| ATOM | 9129 | C | VAL I | 301 | 1.084 | −18.962 | 8.695 | 1.00 | 3.48 | C | C |
| ATOM | 9130 | O | VAL I | 301 | 0.647 | −20.061 | 9.037 | 1.00 | 3.92 | C | O |
| ATOM | 9131 | N | GLN I | 302 | 1.466 | −18.684 | 7.456 | 1.00 | 3.21 | C | N |
| ATOM | 9132 | CA | GLN I | 302 | 1.274 | −19.625 | 6.368 | 1.00 | 3.01 | C | C |
| ATOM | 9133 | CB | GLN I | 302 | 0.090 | −19.202 | 5.504 | 1.00 | 3.24 | C | C |
| ATOM | 9134 | CG | GLN I | 302 | −0.067 | −20.021 | 4.239 | 1.00 | 5.21 | C | C |
| ATOM | 9135 | CD | GLN I | 302 | −1.479 | −19.984 | 3.701 | 1.00 | 7.54 | C | C |
| ATOM | 9136 | OE1 | GLN I | 302 | −2.435 | −19.784 | 4.449 | 1.00 | 7.28 | C | O |
| ATOM | 9137 | NE2 | GLN I | 302 | −1.616 | −20.143 | 2.390 | 1.00 | 9.27 | C | N |
| ATOM | 9138 | C | GLN I | 302 | 2.533 | −19.693 | 5.523 | 1.00 | 2.42 | C | C |
| ATOM | 9139 | O | GLN I | 302 | 2.881 | −18.736 | 4.832 | 1.00 | 2.40 | C | O |
| ATOM | 9140 | N | LEU I | 303 | 3.292 | −20.765 | 5.707 | 1.00 | 2.00 | C | N |
| ATOM | 9141 | CA | LEU I | 303 | 4.607 | −20.868 | 5.104 | 1.00 | 2.00 | C | C |
| ATOM | 9142 | CB | LEU I | 303 | 5.628 | −21.358 | 6.130 | 1.00 | 2.00 | C | C |
| ATOM | 9143 | CG | LEU I | 303 | 5.663 | −20.559 | 7.434 | 1.00 | 2.00 | C | C |
| ATOM | 9144 | CD1 | LEU I | 303 | 6.712 | −21.118 | 8.382 | 1.00 | 2.00 | C | C |
| ATOM | 9145 | CD2 | LEU I | 303 | 5.918 | −19.085 | 7.154 | 1.00 | 2.00 | C | C |
| ATOM | 9146 | C | LEU I | 303 | 4.564 | −21.805 | 3.909 | 1.00 | 2.00 | C | C |
| ATOM | 9147 | O | LEU I | 303 | 3.658 | −22.630 | 3.794 | 1.00 | 2.45 | C | O |
| ATOM | 9148 | N | PRO I | 304 | 5.499 | −21.620 | 2.970 | 1.00 | 2.00 | C | N |
| ATOM | 9149 | CA | PRO I | 304 | 5.617 | −22.519 | 1.840 | 1.00 | 2.07 | C | C |
| ATOM | 9150 | CB | PRO I | 304 | 6.326 | −21.656 | 0.803 | 1.00 | 2.00 | C | C |
| ATOM | 9151 | CG | PRO I | 304 | 7.221 | −20.775 | 1.620 | 1.00 | 2.00 | C | C |
| ATOM | 9152 | CD | PRO I | 304 | 6.564 | −20.601 | 2.973 | 1.00 | 2.06 | C | C |
| ATOM | 9153 | C | PRO I | 304 | 6.492 | −23.711 | 2.194 | 1.00 | 2.11 | C | C |
| ATOM | 9154 | O | PRO I | 304 | 7.544 | −23.530 | 2.809 | 1.00 | 2.13 | C | O |
| ATOM | 9155 | N | ALA I | 305 | 5.903 | −24.902 | 2.097 | 1.00 | 30.00 | C | N |
| ATOM | 9156 | CA | ALA I | 305 | 6.371 | −25.959 | 1.180 | 1.00 | 30.00 | C | C |
| ATOM | 9157 | CB | ALA I | 305 | 5.841 | −27.314 | 1.627 | 1.00 | 30.00 | C | C |
| ATOM | 9158 | C | ALA I | 305 | 6.011 | −25.690 | −0.310 | 1.00 | 30.00 | C | C |
| ATOM | 9159 | O | ALA I | 305 | 5.614 | −24.571 | −0.624 | 1.00 | 30.00 | C | O |
| ATOM | 9160 | N | TYR I | 306 | 5.840 | −26.756 | −1.125 | 1.00 | 13.49 | C | N |
| ATOM | 9161 | CA | TYR I | 306 | 6.433 | −26.943 | −2.522 | 1.00 | 13.70 | C | C |
| ATOM | 9162 | CB | TYR I | 306 | 7.412 | −28.117 | −2.435 | 1.00 | 13.93 | C | C |
| ATOM | 9163 | CG | TYR I | 306 | 8.486 | −28.217 | −3.491 | 1.00 | 14.08 | C | C |
| ATOM | 9164 | CD1 | TYR I | 306 | 8.717 | −29.447 | −4.102 | 1.00 | 14.59 | C | C |
| ATOM | 9165 | CE1 | TYR I | 306 | 9.982 | −29.819 | −4.531 | 1.00 | 14.82 | C | C |
| ATOM | 9166 | CZ | TYR I | 306 | 11.002 | −28.893 | −4.523 | 1.00 | 14.52 | C | C |
| ATOM | 9167 | OH | TYR I | 306 | 12.189 | −29.164 | −5.167 | 1.00 | 13.02 | C | O |
| ATOM | 9168 | CE2 | TYR I | 306 | 10.781 | −27.632 | −4.011 | 1.00 | 15.22 | C | C |
| ATOM | 9169 | CD2 | TYR I | 306 | 9.534 | −27.303 | −3.495 | 1.00 | 14.61 | C | C |
| ATOM | 9170 | C | TYR I | 306 | 5.391 | −27.297 | −3.661 | 1.00 | 13.68 | C | C |
| ATOM | 9171 | O | TYR I | 306 | 4.296 | −27.738 | −3.325 | 1.00 | 13.95 | C | O |
| ATOM | 9172 | N | GLY I | 307 | 5.786 | −27.316 | −4.962 | 1.00 | 13.54 | C | N |
| ATOM | 9173 | CA | GLY I | 307 | 5.057 | −28.108 | −6.046 | 1.00 | 13.37 | C | C |
| ATOM | 9174 | C | GLY I | 307 | 5.290 | −27.995 | −7.585 | 1.00 | 13.47 | C | C |
| ATOM | 9175 | O | GLY I | 307 | 5.871 | −27.017 | −8.054 | 1.00 | 13.90 | C | O |
| ATOM | 9176 | N | VAL I | 308 | 4.808 | −28.976 | −8.370 | 1.00 | 13.31 | C | N |
| ATOM | 9177 | CA | VAL I | 308 | 3.931 | −28.717 | −9.548 | 1.00 | 13.30 | C | C |
| ATOM | 9178 | CB | VAL I | 308 | 3.187 | −27.383 | −9.404 | 1.00 | 13.03 | C | C |
| ATOM | 9179 | CG1 | VAL I | 308 | 1.935 | −27.380 | −10.261 | 1.00 | 13.62 | C | C |
| ATOM | 9180 | CG2 | VAL I | 308 | 2.849 | −27.116 | −7.951 | 1.00 | 12.86 | C | C |
| ATOM | 9181 | C | VAL I | 308 | 4.596 | −28.668 | −10.927 | 1.00 | 13.58 | C | C |
| ATOM | 9182 | O | VAL I | 308 | 5.407 | −29.530 | −11.271 | 1.00 | 13.71 | C | O |
| ATOM | 9183 | N | ILE I | 309 | 4.541 | −27.470 | −11.498 | 1.00 | 4.08 | C | N |
| ATOM | 9184 | CA | ILE I | 309 | 5.611 | −26.532 | −11.251 | 1.00 | 4.84 | C | C |
| ATOM | 9185 | CB | ILE I | 309 | 6.204 | −26.810 | −9.854 | 1.00 | 4.89 | C | C |
| ATOM | 9186 | CG1 | ILE I | 309 | 5.086 | −26.815 | −8.801 | 1.00 | 5.85 | C | C |
| ATOM | 9187 | CD1 | ILE I | 309 | 4.233 | −25.561 | −8.803 | 1.00 | 8.12 | C | C |
| ATOM | 9188 | CG2 | ILE I | 309 | 7.308 | −25.817 | −9.517 | 1.00 | 4.44 | C | C |
| ATOM | 9189 | C | ILE I | 309 | 6.637 | −26.814 | −12.345 | 1.00 | 5.23 | C | C |
| ATOM | 9190 | O | ILE I | 309 | 6.299 | −27.437 | −13.352 | 1.00 | 5.92 | C | O |
| ATOM | 9191 | N | ASP I | 310 | 7.864 | −26.330 | −12.187 | 1.00 | 5.33 | C | N |
| ATOM | 9192 | CA | ASP I | 310 | 9.004 | −26.924 | −12.889 | 1.00 | 5.83 | C | C |
| ATOM | 9193 | CB | ASP I | 310 | 9.086 | −28.423 | −12.589 | 1.00 | 6.21 | C | C |
| ATOM | 9194 | CG | ASP I | 310 | 9.193 | −28.716 | −11.104 | 1.00 | 7.19 | C | C |
| ATOM | 9195 | OD1 | ASP I | 310 | 8.350 | −29.478 | −10.583 | 1.00 | 7.74 | C | O |
| ATOM | 9196 | OD2 | ASP I | 310 | 10.151 | −28.228 | −10.467 | 1.00 | 7.58 | C | O |
| ATOM | 9197 | C | ASP I | 310 | 8.997 | −26.705 | −14.408 | 1.00 | 6.02 | C | C |
| ATOM | 9198 | O | ASP I | 310 | 9.656 | −27.438 | −15.146 | 1.00 | 6.05 | C | O |
| ATOM | 9199 | N | THR I | 311 | 8.273 | −25.687 | −14.866 | 1.00 | 6.24 | C | N |
| ATOM | 9200 | CA | THR I | 311 | 8.429 | −25.173 | −16.229 | 1.00 | 6.14 | C | C |
| ATOM | 9201 | CB | THR I | 311 | 7.231 | −24.299 | −16.630 | 1.00 | 5.69 | C | C |
| ATOM | 9202 | OG1 | THR I | 311 | 6.093 | −24.646 | −15.830 | 1.00 | 5.34 | C | O |
| ATOM | 9203 | CG2 | THR I | 311 | 6.897 | −24.500 | −18.092 | 1.00 | 7.10 | C | C |
| ATOM | 9204 | C | THR I | 311 | 9.682 | −24.313 | −16.335 | 1.00 | 6.78 | C | C |
| ATOM | 9205 | O | THR I | 311 | 9.970 | −23.530 | −15.430 | 1.00 | 7.33 | C | O |
| ATOM | 9206 | N | PRO I | 312 | 10.330 | −24.318 | −17.509 | 1.00 | 7.00 | C | N |
| ATOM | 9207 | CA | PRO I | 312 | 11.470 | −23.419 | −17.668 | 1.00 | 7.23 | C | C |
| ATOM | 9208 | CB | PRO I | 312 | 12.060 | −23.834 | −19.020 | 1.00 | 7.00 | C | C |
| ATOM | 9209 | CG | PRO I | 312 | 10.936 | −24.505 | −19.747 | 1.00 | 6.59 | C | C |
| ATOM | 9210 | CD | PRO I | 312 | 10.098 | −25.159 | −18.697 | 1.00 | 6.99 | C | C |
| ATOM | 9211 | C | PRO I | 312 | 11.016 | −21.962 | −17.703 | 1.00 | 7.69 | C | C |
| ATOM | 9212 | O | PRO I | 312 | 9.943 | −21.670 | −18.228 | 1.00 | 7.55 | C | O |
| ATOM | 9213 | N | CYS I | 313 | 11.755 | −21.077 | −17.041 | 1.00 | 8.10 | C | N |
| ATOM | 9214 | CA | CYS I | 313 | 11.668 | −19.654 | −17.353 | 1.00 | 8.54 | C | C |
| ATOM | 9215 | CB | CYS I | 313 | 11.082 | −18.852 | −16.186 | 1.00 | 8.77 | C | C |
| ATOM | 9216 | SG | CYS I | 313 | 10.025 | −19.785 | −15.057 | 1.00 | 10.74 | C | S |
| ATOM | 9217 | C | CYS I | 313 | 13.000 | −19.058 | −17.780 | 1.00 | 8.19 | C | C |
| ATOM | 9218 | O | CYS I | 313 | 14.035 | −19.725 | −17.760 | 1.00 | 8.07 | C | O |
| ATOM | 9219 | N | TRP I | 314 | 12.964 | −17.777 | −18.126 | 1.00 | 7.93 | C | N |
| ATOM | 9220 | CA | TRP I | 314 | 14.162 | −17.035 | −18.473 | 1.00 | 7.80 | C | C |
| ATOM | 9221 | CB | TRP I | 314 | 14.563 | −17.322 | −19.919 | 1.00 | 8.13 | C | C |
| ATOM | 9222 | CG | TRP I | 314 | 13.457 | −17.091 | −20.902 | 1.00 | 9.09 | C | C |
| ATOM | 9223 | CD1 | TRP I | 314 | 13.295 | −16.003 | −21.710 | 1.00 | 10.26 | C | C |
| ATOM | 9224 | NE1 | TRP I | 314 | 12.219 | −16.189 | −22.546 | 1.00 | 10.86 | C | N |
| ATOM | 9225 | CE2 | TRP I | 314 | 11.671 | −17.421 | −22.298 | 1.00 | 10.68 | C | C |
| ATOM | 9226 | CD2 | TRP I | 314 | 12.426 | −18.020 | −21.269 | 1.00 | 10.49 | C | C |
| ATOM | 9227 | CE3 | TRP I | 314 | 12.083 | −19.308 | −20.842 | 1.00 | 11.67 | C | C |
| ATOM | 9228 | CZ3 | TRP I | 314 | 11.009 | −19.946 | −21.447 | 1.00 | 12.98 | C | C |
| ATOM | 9229 | CH2 | TRP I | 314 | 10.268 | −19.318 | −22.457 | 1.00 | 12.74 | C | C |
| ATOM | 9230 | CZ2 | TRP I | 314 | 10.580 | −18.059 | −22.895 | 1.00 | 11.73 | C | C |
| ATOM | 9231 | C | TRP I | 314 | 13.899 | −15.550 | −18.298 | 1.00 | 7.34 | C | C |
| ATOM | 9232 | O | TRP I | 314 | 12.764 | −15.091 | −18.427 | 1.00 | 7.47 | C | O |
| ATOM | 9233 | N | LYS I | 315 | 14.936 | −14.815 | −17.920 | 1.00 | 6.75 | C | N |
| ATOM | 9234 | CA | LYS I | 315 | 14.833 | −13.372 | −17.823 | 1.00 | 6.37 | C | C |
| ATOM | 9235 | CB | LYS I | 315 | 15.493 | −12.875 | −16.541 | 1.00 | 6.38 | C | C |
| ATOM | 9236 | CG | LYS I | 315 | 14.992 | −11.522 | −16.065 | 1.00 | 6.55 | C | C |
| ATOM | 9237 | CD | LYS I | 315 | 15.692 | −11.117 | −14.778 | 1.00 | 8.01 | C | C |
| ATOM | 9238 | CE | LYS I | 315 | 15.198 | −9.776 | −14.269 | 1.00 | 8.78 | C | C |
| ATOM | 9239 | NZ | LYS I | 315 | 16.141 | −9.189 | −13.276 | 1.00 | 10.21 | C | N |
| ATOM | 9240 | C | LYS I | 315 | 15.468 | −12.711 | −19.036 | 1.00 | 6.32 | C | C |
| ATOM | 9241 | O | LYS I | 315 | 16.510 | −13.153 | −19.523 | 1.00 | 6.21 | C | O |
| ATOM | 9242 | N | LEU I | 316 | 14.766 | −11.724 | −19.584 | 1.00 | 6.58 | C | N |
| ATOM | 9243 | CA | LEU I | 316 | 15.231 | −10.992 | −20.758 | 1.00 | 7.02 | C | C |
| ATOM | 9244 | CB | LEU I | 316 | 14.122 | −10.908 | −21.809 | 1.00 | 6.64 | C | C |
| ATOM | 9245 | CG | LEU I | 316 | 14.308 | −9.874 | −22.922 | 1.00 | 6.21 | C | C |
| ATOM | 9246 | CD1 | LEU I | 316 | 15.563 | −10.150 | −23.731 | 1.00 | 5.94 | C | C |
| ATOM | 9247 | CD2 | LEU I | 316 | 13.082 | −9.824 | −23.819 | 1.00 | 6.57 | C | C |
| ATOM | 9248 | C | LEU I | 316 | 15.675 | −9.588 | −20.372 | 1.00 | 7.77 | C | C |
| ATOM | 9249 | O | LEU I | 316 | 14.865 | −8.768 | −19.941 | 1.00 | 8.47 | C | O |
| ATOM | 9250 | N | HIS I | 317 | 16.956 | −9.302 | −20.563 | 1.00 | 8.05 | C | N |
| ATOM | 9251 | CA | HIS I | 317 | 17.422 | −7.929 | −20.547 | 1.00 | 8.21 | C | C |
| ATOM | 9252 | CB | HIS I | 317 | 18.641 | −7.786 | −19.640 | 1.00 | 9.04 | C | C |
| ATOM | 9253 | CG | HIS I | 317 | 18.644 | −8.734 | −18.483 | 1.00 | 11.41 | C | C |
| ATOM | 9254 | ND1 | HIS I | 317 | 18.314 | −8.347 | −17.202 | 1.00 | 13.62 | C | N |
| ATOM | 9255 | CE1 | HIS I | 317 | 18.423 | −9.383 | −16.389 | 1.00 | 14.91 | C | C |
| ATOM | 9256 | NE2 | HIS I | 317 | 18.811 | −10.428 | −17.097 | 1.00 | 14.25 | C | N |
| ATOM | 9257 | CD2 | HIS I | 317 | 18.960 | −10.048 | −18.409 | 1.00 | 12.67 | C | C |
| ATOM | 9258 | C | HIS I | 317 | 17.760 | −7.469 | −21.950 | 1.00 | 7.39 | C | C |
| ATOM | 9259 | O | HIS I | 317 | 17.881 | −8.278 | −22.869 | 1.00 | 7.23 | C | O |
| ATOM | 9260 | N | THR I | 318 | 17.997 | −6.172 | −22.086 | 1.00 | 7.11 | C | N |
| ATOM | 9261 | CA | THR I | 318 | 17.745 | −5.476 | −23.333 | 1.00 | 6.56 | C | C |
| ATOM | 9262 | CB | THR I | 318 | 16.268 | −5.093 | −23.468 | 1.00 | 6.28 | C | C |
| ATOM | 9263 | OG1 | THR I | 318 | 15.652 | −5.909 | −24.471 | 1.00 | 6.33 | C | O |
| ATOM | 9264 | CG2 | THR I | 318 | 16.141 | −3.634 | −23.866 | 1.00 | 6.52 | C | C |
| ATOM | 9265 | C | THR I | 318 | 18.563 | −4.201 | −23.357 | 1.00 | 6.58 | C | C |
| ATOM | 9266 | O | THR I | 318 | 18.437 | −3.356 | −22.472 | 1.00 | 6.67 | C | O |
| ATOM | 9267 | N | SER I | 319 | 19.378 | −4.044 | −24.390 | 1.00 | 6.81 | C | N |
| ATOM | 9268 | CA | SER I | 319 | 20.173 | −2.837 | −24.533 | 1.00 | 7.31 | C | C |
| ATOM | 9269 | CB | SER I | 319 | 21.633 | −3.105 | −24.159 | 1.00 | 7.14 | C | C |
| ATOM | 9270 | OG | SER I | 319 | 22.309 | −1.904 | −23.825 | 1.00 | 6.84 | C | O |
| ATOM | 9271 | C | SER I | 319 | 20.069 | −2.273 | −25.945 | 1.00 | 7.93 | C | C |
| ATOM | 9272 | O | SER I | 319 | 19.736 | −2.996 | −26.887 | 1.00 | 8.65 | C | O |
| ATOM | 9273 | N | PRO I | 320 | 20.242 | −0.952 | −26.075 | 1.00 | 8.08 | C | N |
| ATOM | 9274 | CA | PRO I | 320 | 20.052 | −0.299 | −27.363 | 1.00 | 8.34 | C | C |
| ATOM | 9275 | CB | PRO I | 320 | 20.263 | 1.187 | −27.047 | 1.00 | 7.99 | C | C |
| ATOM | 9276 | CG | PRO I | 320 | 20.887 | 1.228 | −25.680 | 1.00 | 8.01 | C | C |
| ATOM | 9277 | CD | PRO I | 320 | 20.384 | 0.015 | −24.977 | 1.00 | 8.10 | C | C |
| ATOM | 9278 | C | PRO I | 320 | 21.061 | −0.770 | −28.400 | 1.00 | 9.03 | C | C |
| ATOM | 9279 | O | PRO I | 320 | 22.135 | −1.262 | −28.053 | 1.00 | 8.74 | C | O |
| ATOM | 9280 | N | LEU I | 321 | 20.711 | −0.594 | −29.669 | 1.00 | 10.31 | C | N |
| ATOM | 9281 | CA | LEU I | 321 | 21.427 | −1.228 | −30.766 | 1.00 | 11.63 | C | C |
| ATOM | 9282 | CB | LEU I | 321 | 20.792 | −2.576 | −31.105 | 1.00 | 11.31 | C | C |
| ATOM | 9283 | CG | LEU I | 321 | 21.681 | −3.600 | −31.812 | 1.00 | 10.97 | C | C |
| ATOM | 9284 | CD1 | LEU I | 321 | 20.835 | −4.621 | −32.549 | 1.00 | 10.04 | C | C |
| ATOM | 9285 | CD2 | LEU I | 321 | 22.652 | −2.914 | −32.761 | 1.00 | 10.69 | C | C |
| ATOM | 9286 | C | LEU I | 321 | 21.412 | −0.317 | −31.989 | 1.00 | 12.92 | C | C |
| ATOM | 9287 | O | LEU I | 321 | 20.351 | 0.114 | −32.441 | 1.00 | 13.29 | C | O |
| ATOM | 9288 | N | CYS I | 322 | 22.601 | 0.013 | −32.486 | 1.00 | 13.39 | C | N |
| ATOM | 9289 | CA | CYS I | 322 | 22.751 | 0.987 | −33.563 | 1.00 | 14.16 | C | C |
| ATOM | 9290 | CB | CYS I | 322 | 23.359 | 2.288 | −33.036 | 1.00 | 13.72 | C | C |
| ATOM | 9291 | SG | CYS I | 322 | 22.632 | 2.898 | −31.504 | 1.00 | 13.42 | C | S |
| ATOM | 9292 | C | CYS I | 322 | 23.622 | 0.441 | −34.690 | 1.00 | 15.46 | C | C |
| ATOM | 9293 | O | CYS I | 322 | 24.646 | −0.196 | −34.446 | 1.00 | 15.94 | C | O |
| ATOM | 9294 | N | THR I | 323 | 23.265 | 0.802 | −35.919 | 1.00 | 16.70 | C | N |
| ATOM | 9295 | CA | THR I | 323 | 24.152 | 0.675 | −37.075 | 1.00 | 17.84 | C | C |
| ATOM | 9296 | CB | THR I | 323 | 23.429 | 1.117 | −38.359 | 1.00 | 17.50 | C | C |
| ATOM | 9297 | OG1 | THR I | 323 | 22.416 | 2.076 | −38.031 | 1.00 | 15.70 | C | O |
| ATOM | 9298 | CG2 | THR I | 323 | 22.786 | −0.076 | −39.047 | 1.00 | 18.05 | C | C |
| ATOM | 9299 | C | THR I | 323 | 25.403 | 1.536 | −36.918 | 1.00 | 19.54 | C | C |
| ATOM | 9300 | O | THR I | 323 | 25.408 | 2.477 | −36.124 | 1.00 | 19.84 | C | O |
| ATOM | 9301 | N | THR I | 324 | 26.352 | 1.379 | −37.840 | 1.00 | 21.17 | C | N |
| ATOM | 9302 | CA | THR I | 324 | 27.430 | 2.358 | −37.967 | 1.00 | 22.89 | C | C |
| ATOM | 9303 | CB | THR I | 324 | 28.687 | 1.950 | −37.188 | 1.00 | 22.69 | C | C |
| ATOM | 9304 | OG1 | THR I | 324 | 28.403 | 0.800 | −36.381 | 1.00 | 22.10 | C | O |
| ATOM | 9305 | CG2 | THR I | 324 | 29.150 | 3.094 | −36.300 | 1.00 | 22.03 | C | C |
| ATOM | 9306 | C | THR I | 324 | 27.815 | 2.806 | −39.378 | 1.00 | 24.39 | C | C |
| ATOM | 9307 | O | THR I | 324 | 28.495 | 3.820 | −39.537 | 1.00 | 24.53 | C | O |
| ATOM | 9308 | N | ASN I | 325 | 27.330 | 2.106 | −40.398 | 1.00 | 25.72 | C | N |
| ATOM | 9309 | CA | ASN I | 325 | 27.321 | 2.661 | −41.751 | 1.00 | 27.01 | C | C |
| ATOM | 9310 | CB | ASN I | 325 | 28.628 | 2.344 | −42.484 | 1.00 | 27.37 | C | C |
| ATOM | 9311 | CG | ASN I | 325 | 29.720 | 3.360 | −42.199 | 1.00 | 28.46 | C | C |
| ATOM | 9312 | OD1 | ASN I | 325 | 29.982 | 4.252 | −43.007 | 1.00 | 30.37 | C | O |
| ATOM | 9313 | ND2 | ASN I | 325 | 30.364 | 3.229 | −41.045 | 1.00 | 28.78 | C | N |
| ATOM | 9314 | C | ASN I | 325 | 26.125 | 2.199 | −42.576 | 1.00 | 27.36 | C | C |
| ATOM | 9315 | O | ASN I | 325 | 25.455 | 1.228 | −42.226 | 1.00 | 27.94 | C | O |
| ATOM | 9316 | N | SER I | 330 | 24.776 | 3.883 | −38.461 | 1.00 | 35.76 | C | N |
| ATOM | 9317 | CA | SER I | 330 | 24.479 | 5.303 | −38.606 | 1.00 | 35.01 | C | C |
| ATOM | 9318 | CB | SER I | 330 | 24.922 | 5.803 | −39.983 | 1.00 | 35.47 | C | C |
| ATOM | 9319 | OG | SER I | 330 | 24.296 | 5.067 | −41.019 | 1.00 | 33.31 | C | O |
| ATOM | 9320 | C | SER I | 330 | 22.992 | 5.574 | −38.405 | 1.00 | 34.35 | C | C |
| ATOM | 9321 | O | SER I | 330 | 22.148 | 4.769 | −38.798 | 1.00 | 34.33 | C | O |
| ATOM | 9322 | N | ASN I | 331 | 22.677 | 6.688 | −37.752 | 1.00 | 32.48 | C | N |
| ATOM | 9323 | CA | ASN I | 331 | 21.365 | 7.314 | −37.886 | 1.00 | 30.72 | C | C |
| ATOM | 9324 | CB | ASN I | 331 | 21.065 | 7.642 | −39.357 | 1.00 | 31.31 | C | C |
| ATOM | 9325 | CG | ASN I | 331 | 21.961 | 8.740 | −39.916 | 1.00 | 32.11 | C | C |
| ATOM | 9326 | OD1 | ASN I | 331 | 22.615 | 9.469 | −39.170 | 1.00 | 32.54 | C | O |
| ATOM | 9327 | ND2 | ASN I | 331 | 21.941 | 8.901 | −41.235 | 1.00 | 31.30 | C | N |
| ATOM | 9328 | C | ASN I | 331 | 20.188 | 6.532 | −37.282 | 1.00 | 28.71 | C | C |
| ATOM | 9329 | O | ASN I | 331 | 19.038 | 6.940 | −37.444 | 1.00 | 29.07 | C | O |
| ATOM | 9330 | N | ILE I | 332 | 20.450 | 5.399 | −36.629 | 1.00 | 25.44 | C | N |
| ATOM | 9331 | CA | ILE I | 332 | 19.354 | 4.517 | −36.200 | 1.00 | 21.83 | C | C |
| ATOM | 9332 | CB | ILE I | 332 | 18.777 | 3.711 | −37.387 | 1.00 | 21.42 | C | C |
| ATOM | 9333 | CG1 | ILE I | 332 | 17.534 | 2.929 | −36.961 | 1.00 | 19.89 | C | C |
| ATOM | 9334 | CD1 | ILE I | 332 | 16.273 | 3.385 | −37.652 | 1.00 | 17.67 | C | C |
| ATOM | 9335 | CG2 | ILE I | 332 | 19.828 | 2.777 | −37.965 | 1.00 | 20.61 | C | C |
| ATOM | 9336 | C | ILE I | 332 | 19.679 | 3.570 | −35.035 | 1.00 | 20.37 | C | C |
| ATOM | 9337 | O | ILE I | 332 | 20.593 | 2.751 | −35.134 | 1.00 | 19.95 | C | O |
| ATOM | 9338 | N | CYS I | 333 | 18.816 | 3.561 | −34.019 | 1.00 | 18.27 | C | N |
| ATOM | 9339 | CA | CYS I | 333 | 18.930 | 2.597 | −32.921 | 1.00 | 16.47 | C | C |
| ATOM | 9340 | CB | CYS I | 333 | 19.646 | 3.216 | −31.720 | 1.00 | 16.14 | C | C |
| ATOM | 9341 | SG | CYS I | 333 | 21.144 | 4.124 | −32.136 | 1.00 | 17.09 | C | S |
| ATOM | 9342 | C | CYS I | 333 | 17.615 | 1.958 | −32.475 | 1.00 | 15.73 | C | C |
| ATOM | 9343 | O | CYS I | 333 | 16.556 | 2.586 | −32.511 | 1.00 | 15.43 | C | O |
| ATOM | 9344 | N | LEU I | 334 | 17.761 | 0.811 | −31.819 | 1.00 | 15.06 | C | N |
| ATOM | 9345 | CA | LEU I | 334 | 16.643 | −0.021 | −31.394 | 1.00 | 13.79 | C | C |
| ATOM | 9346 | CB | LEU I | 334 | 16.607 | −1.319 | −32.203 | 1.00 | 13.39 | C | C |
| ATOM | 9347 | CG | LEU I | 334 | 16.482 | −1.230 | −33.725 | 1.00 | 13.93 | C | C |
| ATOM | 9348 | CD1 | LEU I | 334 | 15.861 | −2.496 | −34.287 | 1.00 | 14.20 | C | C |
| ATOM | 9349 | CD2 | LEU I | 334 | 15.686 | −0.009 | −34.151 | 1.00 | 14.52 | C | C |
| ATOM | 9350 | C | LEU I | 334 | 16.852 | −0.366 | −29.928 | 1.00 | 13.22 | C | C |
| ATOM | 9351 | O | LEU I | 334 | 17.968 | −0.685 | −29.520 | 1.00 | 13.31 | C | O |
| ATOM | 9352 | N | THR I | 335 | 15.768 | −0.423 | −29.167 | 1.00 | 12.58 | C | N |
| ATOM | 9353 | CA | THR I | 335 | 15.790 | −1.126 | −27.892 | 1.00 | 12.17 | C | C |
| ATOM | 9354 | CB | THR I | 335 | 15.955 | −0.158 | −26.706 | 1.00 | 12.02 | C | C |
| ATOM | 9355 | OG1 | THR I | 335 | 16.652 | 1.018 | −27.139 | 1.00 | 12.64 | C | O |
| ATOM | 9356 | CG2 | THR I | 335 | 16.739 | −0.825 | −25.582 | 1.00 | 11.47 | C | C |
| ATOM | 9357 | C | THR I | 335 | 14.523 | −1.942 | −27.703 | 1.00 | 11.97 | C | C |
| ATOM | 9358 | O | THR I | 335 | 13.423 | −1.471 | −27.990 | 1.00 | 11.93 | C | O |
| ATOM | 9359 | N | ARG I | 336 | 14.700 | −3.216 | −27.379 | 1.00 | 11.60 | C | N |
| ATOM | 9360 | CA | ARG I | 336 | 13.573 | −4.131 | −27.260 | 1.00 | 12.15 | C | C |
| ATOM | 9361 | CB | ARG I | 336 | 14.066 | −5.579 | −27.235 | 1.00 | 12.16 | C | C |
| ATOM | 9362 | CG | ARG I | 336 | 13.124 | −6.566 | −27.906 | 1.00 | 12.67 | C | C |
| ATOM | 9363 | CD | ARG I | 336 | 13.876 | −7.491 | −28.846 | 1.00 | 12.52 | C | C |
| ATOM | 9364 | NE | ARG I | 336 | 14.414 | −8.649 | −28.144 | 1.00 | 12.35 | C | N |
| ATOM | 9365 | CZ | ARG I | 336 | 13.675 | −9.665 | −27.713 | 1.00 | 12.54 | C | C |
| ATOM | 9366 | NH1 | ARG I | 336 | 12.384 | −9.718 | −28.013 | 1.00 | 12.38 | C | N |
| ATOM | 9367 | NH2 | ARG I | 336 | 14.249 | −10.681 | −27.084 | 1.00 | 12.48 | C | N |
| ATOM | 9368 | C | ARG I | 336 | 12.761 | −3.833 | −26.004 | 1.00 | 12.71 | C | C |
| ATOM | 9369 | O | ARG I | 336 | 13.272 | −3.932 | −24.890 | 1.00 | 12.62 | C | O |
| ATOM | 9370 | N | THR I | 337 | 11.501 | −3.457 | −26.186 | 1.00 | 13.70 | C | N |
| ATOM | 9371 | CA | THR I | 337 | 10.696 | −2.998 | −25.065 | 1.00 | 14.91 | C | C |
| ATOM | 9372 | CB | THR I | 337 | 9.353 | −2.405 | −25.516 | 1.00 | 15.27 | C | C |
| ATOM | 9373 | OG1 | THR I | 337 | 9.571 | −1.474 | −26.584 | 1.00 | 16.65 | C | O |
| ATOM | 9374 | CG2 | THR I | 337 | 8.694 | −1.678 | −24.356 | 1.00 | 15.94 | C | C |
| ATOM | 9375 | C | THR I | 337 | 10.438 | −4.115 | −24.064 | 1.00 | 15.16 | C | C |
| ATOM | 9376 | O | THR I | 337 | 10.515 | −3.902 | −22.857 | 1.00 | 15.60 | C | O |
| ATOM | 9377 | N | ASP I | 338 | 10.036 | −5.277 | −24.566 | 1.00 | 15.15 | C | N |
| ATOM | 9378 | CA | ASP I | 338 | 9.394 | −6.289 | −23.732 | 1.00 | 15.52 | C | C |
| ATOM | 9379 | CB | ASP I | 338 | 8.653 | −7.306 | −24.602 | 1.00 | 16.81 | C | C |
| ATOM | 9380 | CG | ASP I | 338 | 9.242 | −7.417 | −26.000 | 1.00 | 20.57 | C | C |
| ATOM | 9381 | OD1 | ASP I | 338 | 8.554 | −7.950 | −26.895 | 1.00 | 23.00 | C | O |
| ATOM | 9382 | OD2 | ASP I | 338 | 10.395 | −6.981 | −26.200 | 1.00 | 22.92 | C | O |
| ATOM | 9383 | C | ASP I | 338 | 10.402 | −7.004 | −22.837 | 1.00 | 14.21 | C | C |
| ATOM | 9384 | O | ASP I | 338 | 10.930 | −8.057 | −23.196 | 1.00 | 14.00 | C | O |
| ATOM | 9385 | N | ARG I | 339 | 10.654 | −6.432 | −21.664 | 1.00 | 12.79 | C | N |
| ATOM | 9386 | CA | ARG I | 339 | 11.678 | −6.949 | −20.764 | 1.00 | 11.71 | C | C |
| ATOM | 9387 | CB | ARG I | 339 | 12.488 | −5.810 | −20.163 | 1.00 | 11.73 | C | C |
| ATOM | 9388 | CG | ARG I | 339 | 13.374 | −5.080 | −21.129 | 1.00 | 12.49 | C | C |
| ATOM | 9389 | CD | ARG I | 339 | 14.312 | −4.211 | −20.346 | 1.00 | 13.24 | C | C |
| ATOM | 9390 | NE | ARG I | 339 | 14.580 | −2.944 | −21.005 | 1.00 | 12.04 | C | N |
| ATOM | 9391 | CZ | ARG I | 339 | 15.717 | −2.282 | −20.858 | 1.00 | 11.74 | C | C |
| ATOM | 9392 | NH1 | ARG I | 339 | 16.664 | −2.771 | −20.072 | 1.00 | 13.43 | C | N |
| ATOM | 9393 | NH2 | ARG I | 339 | 15.904 | −1.125 | −21.475 | 1.00 | 9.80 | C | N |
| ATOM | 9394 | C | ARG I | 339 | 11.062 | −7.727 | −19.622 | 1.00 | 11.16 | C | C |
| ATOM | 9395 | O | ARG I | 339 | 10.029 | −7.326 | −19.087 | 1.00 | 11.37 | C | O |
| ATOM | 9396 | N | GLY I | 340 | 11.892 | −8.572 | −19.027 | 1.00 | 10.43 | C | N |
| ATOM | 9397 | CA | GLY I | 340 | 11.574 | −9.178 | −17.749 | 1.00 | 9.52 | C | C |
| ATOM | 9398 | C | GLY I | 340 | 11.463 | −10.678 | −17.876 | 1.00 | 8.80 | C | C |
| ATOM | 9399 | O | GLY I | 340 | 12.269 | −11.313 | −18.555 | 1.00 | 8.56 | C | O |
| ATOM | 9400 | N | TRP I | 341 | 10.532 | −11.257 | −17.129 | 1.00 | 8.25 | C | N |
| ATOM | 9401 | CA | TRP I | 341 | 10.462 | −12.699 | −17.005 | 1.00 | 7.98 | C | C |
| ATOM | 9402 | CB | TRP I | 341 | 10.018 | −13.097 | −15.605 | 1.00 | 7.79 | C | C |
| ATOM | 9403 | CG | TRP I | 341 | 11.120 | −13.020 | −14.622 | 1.00 | 7.67 | C | C |
| ATOM | 9404 | CD1 | TRP I | 341 | 11.382 | −11.986 | −13.775 | 1.00 | 8.12 | C | C |
| ATOM | 9405 | NE1 | TRP I | 341 | 12.511 | −12.252 | −13.042 | 1.00 | 8.60 | C | N |
| ATOM | 9406 | CE2 | TRP I | 341 | 13.037 | −13.447 | −13.459 | 1.00 | 7.57 | C | C |
| ATOM | 9407 | CD2 | TRP I | 341 | 12.217 | −13.931 | −14.495 | 1.00 | 7.52 | C | C |
| ATOM | 9408 | CE3 | TRP I | 341 | 12.541 | −15.150 | −15.098 | 1.00 | 7.86 | C | C |
| ATOM | 9409 | CZ3 | TRP I | 341 | 13.653 | −15.837 | −14.650 | 1.00 | 7.14 | C | C |
| ATOM | 9410 | CH2 | TRP I | 341 | 14.457 | −15.323 | −13.625 | 1.00 | 6.39 | C | C |
| ATOM | 9411 | CZ2 | TRP I | 341 | 14.164 | −14.134 | −13.017 | 1.00 | 7.30 | C | C |
| ATOM | 9412 | C | TRP I | 341 | 9.525 | −13.288 | −18.037 | 1.00 | 8.26 | C | C |
| ATOM | 9413 | O | TRP I | 341 | 8.322 | −13.022 | −18.031 | 1.00 | 8.35 | C | O |
| ATOM | 9414 | N | TYR I | 342 | 10.085 | −14.100 | −18.922 | 1.00 | 8.44 | C | N |
| ATOM | 9415 | CA | TYR I | 342 | 9.308 | −15.102 | −19.616 | 1.00 | 8.53 | C | C |
| ATOM | 9416 | CB | TYR I | 342 | 9.755 | −15.202 | −21.069 | 1.00 | 8.43 | C | C |
| ATOM | 9417 | CG | TYR I | 342 | 9.687 | −13.885 | −21.793 | 1.00 | 9.17 | C | C |
| ATOM | 9418 | CD1 | TYR I | 342 | 10.592 | −12.874 | −21.499 | 1.00 | 10.32 | C | C |
| ATOM | 9419 | CE1 | TYR I | 342 | 10.474 | −11.625 | −22.068 | 1.00 | 10.99 | C | C |
| ATOM | 9420 | CZ | TYR I | 342 | 9.395 | −11.349 | −22.873 | 1.00 | 11.00 | C | C |
| ATOM | 9421 | OH | TYR I | 342 | 9.267 | −10.102 | −23.435 | 1.00 | 12.82 | C | O |
| ATOM | 9422 | CE2 | TYR I | 342 | 8.433 | −12.307 | −23.105 | 1.00 | 10.67 | C | C |
| ATOM | 9423 | CD2 | TYR I | 342 | 8.569 | −13.555 | −22.542 | 1.00 | 10.41 | C | C |
| ATOM | 9424 | C | TYR I | 342 | 9.469 | −16.437 | −18.925 | 1.00 | 8.64 | C | C |
| ATOM | 9425 | O | TYR I | 342 | 10.376 | −16.628 | −18.115 | 1.00 | 8.40 | C | O |
| ATOM | 9426 | N | CYS I | 343 | 8.558 | −17.351 | −19.224 | 1.00 | 8.87 | C | N |
| ATOM | 9427 | CA | CYS I | 343 | 8.370 | −18.516 | −18.389 | 1.00 | 9.35 | C | C |
| ATOM | 9428 | CB | CYS I | 343 | 8.266 | −18.116 | −16.915 | 1.00 | 9.17 | C | C |
| ATOM | 9429 | SG | CYS I | 343 | 8.138 | −19.512 | −15.769 | 1.00 | 11.88 | C | S |
| ATOM | 9430 | C | CYS I | 343 | 7.151 | −19.312 | −18.831 | 1.00 | 9.42 | C | C |
| ATOM | 9431 | O | CYS I | 343 | 6.039 | −18.788 | −18.900 | 1.00 | 9.96 | C | O |
| ATOM | 9432 | N | ASP I | 344 | 7.408 | −20.545 | −19.254 | 1.00 | 9.36 | C | N |
| ATOM | 9433 | CA | ASP I | 344 | 6.483 | −21.302 | −20.083 | 1.00 | 9.24 | C | C |
| ATOM | 9434 | CB | ASP I | 344 | 7.122 | −22.628 | −20.500 | 1.00 | 9.34 | C | C |
| ATOM | 9435 | CG | ASP I | 344 | 7.796 | −22.551 | −21.856 | 1.00 | 10.72 | C | C |
| ATOM | 9436 | OD1 | ASP I | 344 | 7.123 | −22.161 | −22.833 | 1.00 | 13.93 | C | O |
| ATOM | 9437 | OD2 | ASP I | 344 | 9.003 | −22.864 | −21.942 | 1.00 | 11.87 | C | O |
| ATOM | 9438 | C | ASP I | 344 | 5.198 | −21.576 | −19.320 | 1.00 | 9.09 | C | C |
| ATOM | 9439 | O | ASP I | 344 | 5.233 | −22.061 | −18.192 | 1.00 | 9.39 | C | O |
| ATOM | 9440 | N | ASN I | 345 | 4.067 | −21.385 | −19.988 | 1.00 | 9.13 | C | N |
| ATOM | 9441 | CA | ASN I | 345 | 2.801 | −21.924 | −19.510 | 1.00 | 9.52 | C | C |
| ATOM | 9442 | CB | ASN I | 345 | 1.770 | −20.804 | −19.361 | 1.00 | 9.61 | C | C |
| ATOM | 9443 | CG | ASN I | 345 | 0.742 | −21.101 | −18.293 | 1.00 | 9.21 | C | C |
| ATOM | 9444 | OD1 | ASN I | 345 | 1.075 | −21.251 | −17.118 | 1.00 | 6.21 | C | O |
| ATOM | 9445 | ND2 | ASN I | 345 | −0.511 | −21.247 | −18.706 | 1.00 | 11.05 | C | N |
| ATOM | 9446 | C | ASN I | 345 | 2.271 | −23.022 | −20.427 | 1.00 | 10.06 | C | C |
| ATOM | 9447 | O | ASN I | 345 | 3.005 | −23.546 | −21.267 | 1.00 | 11.10 | C | O |
| ATOM | 9448 | N | ALA I | 346 | 1.017 | −23.411 | −20.223 | 1.00 | 9.74 | C | N |
| ATOM | 9449 | CA | ALA I | 346 | 0.416 | −24.492 | −20.998 | 1.00 | 9.58 | C | C |
| ATOM | 9450 | CB | ALA I | 346 | −1.062 | −24.635 | −20.650 | 1.00 | 9.60 | C | C |
| ATOM | 9451 | C | ALA I | 346 | 0.593 | −24.278 | −22.502 | 1.00 | 9.66 | C | C |
| ATOM | 9452 | O | ALA I | 346 | −0.241 | −23.639 | −23.145 | 1.00 | 9.39 | C | O |
| ATOM | 9453 | N | GLY I | 347 | 1.655 | −24.849 | −23.065 | 1.00 | 10.04 | C | N |
| ATOM | 9454 | CA | GLY I | 347 | 1.836 | −24.857 | −24.513 | 1.00 | 10.68 | C | C |
| ATOM | 9455 | C | GLY I | 347 | 1.879 | −23.454 | −25.085 | 1.00 | 10.90 | C | C |
| ATOM | 9456 | O | GLY I | 347 | 1.661 | −23.251 | −26.280 | 1.00 | 11.46 | C | O |
| ATOM | 9457 | N | SER I | 348 | 2.016 | −22.477 | −24.193 | 1.00 | 10.30 | C | N |
| ATOM | 9458 | CA | SER I | 348 | 2.256 | −21.088 | −24.567 | 1.00 | 9.94 | C | C |
| ATOM | 9459 | CB | SER I | 348 | 0.933 | −20.322 | −24.613 | 1.00 | 9.88 | C | C |
| ATOM | 9460 | OG | SER I | 348 | 0.000 | −20.969 | −25.461 | 1.00 | 10.09 | C | O |
| ATOM | 9461 | C | SER I | 348 | 3.194 | −20.462 | −23.538 | 1.00 | 9.60 | C | C |
| ATOM | 9462 | O | SER I | 348 | 3.700 | −21.157 | −22.660 | 1.00 | 9.59 | C | O |
| ATOM | 9463 | N | VAL I | 349 | 3.494 | −19.178 | −23.689 | 1.00 | 9.54 | C | N |
| ATOM | 9464 | CA | VAL I | 349 | 4.452 | −18.532 | −22.800 | 1.00 | 9.56 | C | C |
| ATOM | 9465 | CB | VAL I | 349 | 5.700 | −18.047 | −23.558 | 1.00 | 9.28 | C | C |
| ATOM | 9466 | CG1 | VAL I | 349 | 6.722 | −17.476 | −22.589 | 1.00 | 9.24 | C | C |
| ATOM | 9467 | CG2 | VAL I | 349 | 6.303 | −19.183 | −24.370 | 1.00 | 9.94 | C | C |
| ATOM | 9468 | C | VAL I | 349 | 3.832 | −17.367 | −22.041 | 1.00 | 9.59 | C | C |
| ATOM | 9469 | O | VAL I | 349 | 3.179 | −16.506 | −22.631 | 1.00 | 9.69 | C | O |
| ATOM | 9470 | N | SER I | 350 | 4.073 | −17.326 | −20.735 | 1.00 | 9.93 | C | N |
| ATOM | 9471 | CA | SER I | 350 | 3.559 | −16.252 | −19.896 | 1.00 | 10.44 | C | C |
| ATOM | 9472 | CB | SER I | 350 | 2.926 | −16.815 | −18.620 | 1.00 | 10.04 | C | C |
| ATOM | 9473 | OG | SER I | 350 | 1.578 | −17.196 | −18.841 | 1.00 | 8.41 | C | O |
| ATOM | 9474 | C | SER I | 350 | 4.629 | −15.218 | −19.557 | 1.00 | 11.45 | C | C |
| ATOM | 9475 | O | SER I | 350 | 5.746 | −15.561 | −19.168 | 1.00 | 11.58 | C | O |
| ATOM | 9476 | N | PHE I | 351 | 4.259 | −13.948 | −19.679 | 1.00 | 12.28 | C | N |
| ATOM | 9477 | CA | PHE I | 351 | 5.200 | −12.847 | −19.543 | 1.00 | 13.04 | C | C |
| ATOM | 9478 | CB | PHE I | 351 | 5.320 | −12.089 | −20.867 | 1.00 | 12.87 | C | C |
| ATOM | 9479 | CG | PHE I | 351 | 6.094 | −10.809 | −20.763 | 1.00 | 13.52 | C | C |
| ATOM | 9480 | CD1 | PHE I | 351 | 7.344 | −10.788 | −20.172 | 1.00 | 13.76 | C | C |
| ATOM | 9481 | CE1 | PHE I | 351 | 8.066 | −9.613 | −20.085 | 1.00 | 14.10 | C | C |
| ATOM | 9482 | CZ | PHE I | 351 | 7.541 | −8.442 | −20.588 | 1.00 | 14.07 | C | C |
| ATOM | 9483 | CE2 | PHE I | 351 | 6.305 | −8.452 | −21.197 | 1.00 | 14.14 | C | C |
| ATOM | 9484 | CD2 | PHE I | 351 | 5.580 | −9.629 | −21.270 | 1.00 | 13.92 | C | C |
| ATOM | 9485 | C | PHE I | 351 | 4.742 | −11.899 | −18.441 | 1.00 | 13.84 | C | C |
| ATOM | 9486 | O | PHE I | 351 | 3.579 | −11.499 | −18.394 | 1.00 | 13.96 | C | O |
| ATOM | 9487 | N | PHE I | 352 | 5.655 | −11.574 | −17.534 | 1.00 | 14.72 | C | N |
| ATOM | 9488 | CA | PHE I | 352 | 5.344 | −10.699 | −16.412 | 1.00 | 15.98 | C | C |
| ATOM | 9489 | CB | PHE I | 352 | 5.570 | −11.435 | −15.093 | 1.00 | 15.75 | C | C |
| ATOM | 9490 | CG | PHE I | 352 | 5.295 | −12.909 | −15.169 | 1.00 | 15.51 | C | C |
| ATOM | 9491 | CD1 | PHE I | 352 | 6.188 | −13.762 | −15.803 | 1.00 | 15.05 | C | C |
| ATOM | 9492 | CE1 | PHE I | 352 | 5.926 | −15.116 | −15.902 | 1.00 | 14.05 | C | C |
| ATOM | 9493 | CZ | PHE I | 352 | 4.745 | −15.624 | −15.402 | 1.00 | 13.28 | C | C |
| ATOM | 9494 | CE2 | PHE I | 352 | 3.833 | −14.777 | −14.796 | 1.00 | 13.71 | C | C |
| ATOM | 9495 | CD2 | PHE I | 352 | 4.099 | −13.425 | −14.704 | 1.00 | 14.94 | C | C |
| ATOM | 9496 | C | PHE I | 352 | 6.195 | −9.438 | −16.453 | 1.00 | 17.56 | C | C |
| ATOM | 9497 | O | PHE I | 352 | 7.419 | −9.509 | −16.360 | 1.00 | 17.74 | C | O |
| ATOM | 9498 | N | PRO I | 353 | 5.541 | −8.271 | −16.525 | 1.00 | 19.05 | C | N |
| ATOM | 9499 | CA | PRO I | 353 | 6.188 | −7.074 | −17.055 | 1.00 | 20.40 | C | C |
| ATOM | 9500 | CB | PRO I | 353 | 5.010 | −6.146 | −17.365 | 1.00 | 20.26 | C | C |
| ATOM | 9501 | CG | PRO I | 353 | 3.839 | −7.052 | −17.522 | 1.00 | 19.71 | C | C |
| ATOM | 9502 | CD | PRO I | 353 | 4.073 | −8.155 | −16.545 | 1.00 | 18.97 | C | C |
| ATOM | 9503 | C | PRO I | 353 | 7.136 | −6.408 | −16.058 | 1.00 | 21.94 | C | C |
| ATOM | 9504 | O | PRO I | 353 | 8.087 | −5.745 | −16.470 | 1.00 | 22.29 | C | O |
| ATOM | 9505 | N | GLN I | 354 | 6.858 | −6.544 | −14.763 | 1.00 | 23.38 | C | N |
| ATOM | 9506 | CA | GLN I | 354 | 7.646 | −5.847 | −13.743 | 1.00 | 24.64 | C | C |
| ATOM | 9507 | CB | GLN I | 354 | 6.943 | −4.569 | −13.274 | 1.00 | 24.93 | C | C |
| ATOM | 9508 | CG | GLN I | 354 | 5.458 | −4.509 | −13.587 | 1.00 | 26.00 | C | C |
| ATOM | 9509 | CD | GLN I | 354 | 4.646 | −5.481 | −12.756 | 1.00 | 25.92 | C | C |
| ATOM | 9510 | OE1 | GLN I | 354 | 4.710 | −5.474 | −11.527 | 1.00 | 25.67 | C | O |
| ATOM | 9511 | NE2 | GLN I | 354 | 3.870 | −6.324 | −13.427 | 1.00 | 24.27 | C | N |
| ATOM | 9512 | C | GLN I | 354 | 8.048 | −6.707 | −12.543 | 1.00 | 24.85 | C | C |
| ATOM | 9513 | O | GLN I | 354 | 7.280 | −7.556 | −12.088 | 1.00 | 24.51 | C | O |
| ATOM | 9514 | N | ALA I | 355 | 9.200 | −6.384 | −11.959 | 1.00 | 25.22 | C | N |
| ATOM | 9515 | CA | ALA I | 355 | 9.818 | −7.215 | −10.926 | 1.00 | 25.34 | C | C |
| ATOM | 9516 | CB | ALA I | 355 | 10.949 | −6.458 | −10.245 | 1.00 | 25.09 | C | C |
| ATOM | 9517 | C | ALA I | 355 | 8.809 | −7.711 | −9.892 | 1.00 | 24.92 | C | C |
| ATOM | 9518 | O | ALA I | 355 | 8.679 | −8.915 | −9.669 | 1.00 | 25.23 | C | O |
| ATOM | 9519 | N | GLU I | 356 | 8.278 | −6.771 | −9.115 | 1.00 | 23.93 | C | N |
| ATOM | 9520 | CA | GLU I | 356 | 7.089 | −6.998 | −8.295 | 1.00 | 22.77 | C | C |
| ATOM | 9521 | CB | GLU I | 356 | 5.993 | −5.995 | −8.658 | 1.00 | 23.22 | C | C |
| ATOM | 9522 | CG | GLU I | 356 | 6.412 | −4.545 | −8.500 | 1.00 | 26.33 | C | C |
| ATOM | 9523 | CD | GLU I | 356 | 7.250 | −4.314 | −7.258 | 1.00 | 30.93 | C | C |
| ATOM | 9524 | OE1 | GLU I | 356 | 8.490 | −4.231 | −7.380 | 1.00 | 31.97 | C | O |
| ATOM | 9525 | OE2 | GLU I | 356 | 6.668 | −4.227 | −6.156 | 1.00 | 32.21 | C | O |
| ATOM | 9526 | C | GLU I | 356 | 6.547 | −8.419 | −8.377 | 1.00 | 21.31 | C | C |
| ATOM | 9527 | O | GLU I | 356 | 6.338 | −9.070 | −7.354 | 1.00 | 21.01 | C | O |
| ATOM | 9528 | N | THR I | 357 | 6.137 | −8.809 | −9.577 | 1.00 | 19.51 | C | N |
| ATOM | 9529 | CA | THR I | 357 | 5.358 | −10.023 | −9.766 | 1.00 | 17.55 | C | C |
| ATOM | 9530 | CB | THR I | 357 | 4.829 | −10.115 | −11.207 | 1.00 | 17.30 | C | C |
| ATOM | 9531 | OG1 | THR I | 357 | 5.071 | −8.873 | −11.882 | 1.00 | 17.32 | C | O |
| ATOM | 9532 | CG2 | THR I | 357 | 3.335 | −10.387 | −11.203 | 1.00 | 17.72 | C | C |
| ATOM | 9533 | C | THR I | 357 | 6.178 | −11.268 | −9.433 | 1.00 | 16.62 | C | C |
| ATOM | 9534 | O | THR I | 357 | 5.631 | −12.286 | −9.008 | 1.00 | 17.29 | C | O |
| ATOM | 9535 | N | CYS I | 358 | 7.498 | −11.146 | −9.526 | 1.00 | 14.88 | C | N |
| ATOM | 9536 | CA | CYS I | 358 | 8.365 | −12.317 | −9.566 | 1.00 | 13.15 | C | C |
| ATOM | 9537 | CB | CYS I | 358 | 8.955 | −12.501 | −10.961 | 1.00 | 13.04 | C | C |
| ATOM | 9538 | SG | CYS I | 358 | 7.735 | −12.899 | −12.228 | 1.00 | 14.02 | C | S |
| ATOM | 9539 | C | CYS I | 358 | 9.478 | −12.255 | −8.529 | 1.00 | 12.08 | C | C |
| ATOM | 9540 | O | CYS I | 358 | 10.186 | −11.252 | −8.417 | 1.00 | 12.15 | C | O |
| ATOM | 9541 | N | LYS I | 359 | 9.640 | −13.345 | −7.788 | 1.00 | 11.35 | C | N |
| ATOM | 9542 | CA | LYS I | 359 | 10.793 | −13.519 | −6.918 | 1.00 | 11.01 | C | C |
| ATOM | 9543 | CB | LYS I | 359 | 10.349 | −13.675 | −5.460 | 1.00 | 11.16 | C | C |
| ATOM | 9544 | CG | LYS I | 359 | 8.858 | −13.470 | −5.229 | 1.00 | 13.28 | C | C |
| ATOM | 9545 | CD | LYS I | 359 | 8.607 | −12.427 | −4.150 | 1.00 | 16.11 | C | C |
| ATOM | 9546 | CE | LYS I | 359 | 7.124 | −12.287 | −3.850 | 1.00 | 17.91 | C | C |
| ATOM | 9547 | NZ | LYS I | 359 | 6.818 | −11.012 | −3.145 | 1.00 | 20.99 | C | N |
| ATOM | 9548 | C | LYS I | 359 | 11.592 | −14.739 | −7.349 | 1.00 | 10.46 | C | C |
| ATOM | 9549 | O | LYS I | 359 | 11.091 | −15.589 | −8.085 | 1.00 | 10.70 | C | O |
| ATOM | 9550 | N | VAL I | 360 | 12.791 | −14.880 | −6.798 | 1.00 | 9.99 | C | N |
| ATOM | 9551 | CA | VAL I | 360 | 13.720 | −15.885 | −7.282 | 1.00 | 9.84 | C | C |
| ATOM | 9552 | CB | VAL I | 360 | 14.452 | −15.412 | −8.541 | 1.00 | 9.42 | C | C |
| ATOM | 9553 | CG1 | VAL I | 360 | 15.135 | −14.066 | −8.288 | 1.00 | 10.07 | C | C |
| ATOM | 9554 | CG2 | VAL I | 360 | 15.454 | −16.473 | −8.996 | 1.00 | 9.56 | C | C |
| ATOM | 9555 | C | VAL I | 360 | 14.738 | −16.297 | −6.229 | 1.00 | 9.98 | C | C |
| ATOM | 9556 | O | VAL I | 360 | 15.380 | −15.457 | −5.598 | 1.00 | 9.96 | C | O |
| ATOM | 9557 | N | GLN I | 361 | 14.900 | −17.604 | −6.071 | 1.00 | 10.39 | C | N |
| ATOM | 9558 | CA | GLN I | 361 | 15.781 | −18.150 | −5.052 | 1.00 | 11.53 | C | C |
| ATOM | 9559 | CB | GLN I | 361 | 14.966 | −18.692 | −3.880 | 1.00 | 12.03 | C | C |
| ATOM | 9560 | CG | GLN I | 361 | 15.810 | −19.254 | −2.750 | 1.00 | 16.44 | C | C |
| ATOM | 9561 | CD | GLN I | 361 | 15.388 | −18.722 | −1.398 | 1.00 | 25.16 | C | C |
| ATOM | 9562 | OE1 | GLN I | 361 | 14.773 | −19.434 | −0.604 | 1.00 | 29.01 | C | O |
| ATOM | 9563 | NE2 | GLN I | 361 | 15.686 | −17.453 | −1.141 | 1.00 | 27.20 | C | N |
| ATOM | 9564 | C | GLN I | 361 | 16.613 | −19.271 | −5.641 | 1.00 | 11.15 | C | C |
| ATOM | 9565 | O | GLN I | 361 | 16.114 | −20.375 | −5.854 | 1.00 | 11.80 | C | O |
| ATOM | 9566 | N | SER I | 362 | 17.877 | −18.986 | −5.925 | 1.00 | 10.90 | C | N |
| ATOM | 9567 | CA | SER I | 362 | 18.671 | −19.921 | −6.695 | 1.00 | 10.30 | C | C |
| ATOM | 9568 | CB | SER I | 362 | 18.610 | −21.302 | −6.040 | 1.00 | 10.47 | C | C |
| ATOM | 9569 | OG | SER I | 362 | 19.659 | −22.138 | −6.491 | 1.00 | 11.17 | C | O |
| ATOM | 9570 | C | SER I | 362 | 18.088 | −19.980 | −8.103 | 1.00 | 9.58 | C | C |
| ATOM | 9571 | O | SER I | 362 | 17.759 | −18.946 | −8.683 | 1.00 | 9.48 | C | O |
| ATOM | 9572 | N | ASN I | 363 | 17.806 | −21.188 | −8.577 | 1.00 | 9.12 | C | N |
| ATOM | 9573 | CA | ASN I | 363 | 17.229 | −21.365 | −9.904 | 1.00 | 8.73 | C | C |
| ATOM | 9574 | CB | ASN I | 363 | 17.821 | −22.602 | −10.568 | 1.00 | 9.01 | C | C |
| ATOM | 9575 | CG | ASN I | 363 | 17.547 | −23.864 | −9.781 | 1.00 | 9.29 | C | C |
| ATOM | 9576 | OD1 | ASN I | 363 | 16.903 | −23.826 | −8.732 | 1.00 | 9.82 | C | O |
| ATOM | 9577 | ND2 | ASN I | 363 | 17.981 | −25.000 | −10.314 | 1.00 | 10.08 | C | N |
| ATOM | 9578 | C | ASN I | 363 | 15.716 | −21.503 | −9.827 | 1.00 | 8.12 | C | C |
| ATOM | 9579 | O | ASN I | 363 | 15.084 | −22.042 | −10.736 | 1.00 | 8.10 | C | O |
| ATOM | 9580 | N | ARG I | 364 | 15.168 | −21.165 | −8.665 | 1.00 | 7.20 | C | N |
| ATOM | 9581 | CA | ARG I | 364 | 13.731 | −21.222 | −8.445 | 1.00 | 6.82 | C | C |
| ATOM | 9582 | CB | ARG I | 364 | 13.427 | −21.658 | −7.012 | 1.00 | 6.91 | C | C |
| ATOM | 9583 | CG | ARG I | 364 | 12.287 | −22.649 | −6.908 | 1.00 | 7.92 | C | C |
| ATOM | 9584 | CD | ARG I | 364 | 12.520 | −23.817 | −7.843 | 1.00 | 8.20 | C | C |
| ATOM | 9585 | NE | ARG I | 364 | 11.374 | −24.061 | −8.711 | 1.00 | 8.70 | C | N |
| ATOM | 9586 | CZ | ARG I | 364 | 11.056 | −25.255 | −9.196 | 1.00 | 8.88 | C | C |
| ATOM | 9587 | NH1 | ARG I | 364 | 11.800 | −26.309 | −8.894 | 1.00 | 8.57 | C | N |
| ATOM | 9588 | NH2 | ARG I | 364 | 9.976 | −25.403 | −9.950 | 1.00 | 10.59 | C | N |
| ATOM | 9589 | C | ARG I | 364 | 13.110 | −19.861 | −8.694 | 1.00 | 6.58 | C | C |
| ATOM | 9590 | O | ARG I | 364 | 13.540 | −18.861 | −8.121 | 1.00 | 6.65 | C | O |
| ATOM | 9591 | N | VAL I | 365 | 12.031 | −19.846 | −9.466 | 1.00 | 6.64 | C | N |
| ATOM | 9592 | CA | VAL I | 365 | 11.325 | −18.609 | −9.756 | 1.00 | 6.54 | C | C |
| ATOM | 9593 | CB | VAL I | 365 | 11.399 | −18.261 | −11.254 | 1.00 | 5.95 | C | C |
| ATOM | 9594 | CG1 | VAL I | 365 | 10.374 | −17.196 | −11.608 | 1.00 | 5.48 | C | C |
| ATOM | 9595 | CG2 | VAL I | 365 | 12.801 | −17.804 | −11.623 | 1.00 | 6.22 | C | C |
| ATOM | 9596 | C | VAL I | 365 | 9.866 | −18.723 | −9.335 | 1.00 | 7.35 | C | C |
| ATOM | 9597 | O | VAL I | 365 | 9.271 | −19.799 | −9.416 | 1.00 | 8.33 | C | O |
| ATOM | 9598 | N | PHE I | 366 | 9.309 | −17.619 | −8.843 | 1.00 | 7.37 | C | N |
| ATOM | 9599 | CA | PHE I | 366 | 7.919 | −17.584 | −8.401 | 1.00 | 7.44 | C | C |
| ATOM | 9600 | CB | PHE I | 366 | 7.840 | −17.604 | −6.875 | 1.00 | 7.30 | C | C |
| ATOM | 9601 | CG | PHE I | 366 | 8.426 | −18.834 | −6.255 | 1.00 | 6.70 | C | C |
| ATOM | 9602 | CD1 | PHE I | 366 | 9.799 | −18.991 | −6.163 | 1.00 | 6.70 | C | C |
| ATOM | 9603 | CE1 | PHE I | 366 | 10.345 | −20.120 | −5.588 | 1.00 | 5.94 | C | C |
| ATOM | 9604 | CZ | PHE I | 366 | 9.520 | −21.083 | −5.055 | 1.00 | 5.71 | C | C |
| ATOM | 9605 | CE2 | PHE I | 366 | 8.149 | −20.934 | −5.130 | 1.00 | 5.96 | C | C |
| ATOM | 9606 | CD2 | PHE I | 366 | 7.609 | −19.805 | −5.710 | 1.00 | 6.52 | C | C |
| ATOM | 9607 | C | PHE I | 366 | 7.236 | −16.335 | −8.919 | 1.00 | 7.45 | C | C |
| ATOM | 9608 | O | PHE I | 366 | 7.530 | −15.225 | −8.476 | 1.00 | 7.45 | C | O |
| ATOM | 9609 | N | CYS I | 367 | 6.282 | −16.524 | −9.819 | 1.00 | 8.00 | C | N |
| ATOM | 9610 | CA | CYS I | 367 | 5.626 | −15.400 | −10.450 | 1.00 | 8.90 | C | C |
| ATOM | 9611 | CB | CYS I | 367 | 5.985 | −15.338 | −11.934 | 1.00 | 9.46 | C | C |
| ATOM | 9612 | SG | CYS I | 367 | 7.712 | −14.929 | −12.259 | 1.00 | 10.79 | C | S |
| ATOM | 9613 | C | CYS I | 367 | 4.121 | −15.484 | −10.268 | 1.00 | 9.09 | C | C |
| ATOM | 9614 | O | CYS I | 367 | 3.591 | −16.493 | −9.799 | 1.00 | 9.24 | C | O |
| ATOM | 9615 | N | ASP I | 368 | 3.449 | −14.382 | −10.568 | 1.00 | 9.54 | C | N |
| ATOM | 9616 | CA | ASP I | 368 | 2.034 | −14.255 | −10.285 | 1.00 | 10.55 | C | C |
| ATOM | 9617 | CB | ASP I | 368 | 1.792 | −13.067 | −9.349 | 1.00 | 10.53 | C | C |
| ATOM | 9618 | CG | ASP I | 368 | 0.421 | −13.102 | −8.698 | 1.00 | 11.11 | C | C |
| ATOM | 9619 | OD1 | ASP I | 368 | −0.574 | −13.335 | −9.415 | 1.00 | 11.42 | C | O |
| ATOM | 9620 | OD2 | ASP I | 368 | 0.323 | −12.765 | −7.499 | 1.00 | 11.35 | C | O |
| ATOM | 9621 | C | ASP I | 368 | 1.266 | −14.073 | −11.590 | 1.00 | 11.45 | C | C |
| ATOM | 9622 | O | ASP I | 368 | 1.418 | −13.061 | −12.274 | 1.00 | 12.05 | C | O |
| ATOM | 9623 | N | THR I | 369 | 0.563 | −15.122 | −12.003 | 1.00 | 12.41 | C | N |
| ATOM | 9624 | CA | THR I | 369 | −0.399 | −15.013 | −13.089 | 1.00 | 13.00 | C | C |
| ATOM | 9625 | CB | THR I | 369 | −1.513 | −16.063 | −12.953 | 1.00 | 12.69 | C | C |
| ATOM | 9626 | OG1 | THR I | 369 | −0.935 | −17.374 | −12.929 | 1.00 | 11.72 | C | O |
| ATOM | 9627 | CG2 | THR I | 369 | −2.488 | −15.962 | −14.115 | 1.00 | 13.33 | C | C |
| ATOM | 9628 | C | THR I | 369 | −1.034 | −13.632 | −13.093 | 1.00 | 14.13 | C | C |
| ATOM | 9629 | O | THR I | 369 | −0.895 | −12.874 | −14.051 | 1.00 | 14.72 | C | O |
| ATOM | 9630 | N | MET I | 370 | −1.675 | −13.289 | −11.984 | 1.00 | 15.25 | C | N |
| ATOM | 9631 | CA | MET I | 370 | −2.681 | −12.240 | −11.975 | 1.00 | 16.60 | C | C |
| ATOM | 9632 | CB | MET I | 370 | −2.538 | −11.361 | −10.734 | 1.00 | 17.42 | C | C |
| ATOM | 9633 | CG | MET I | 370 | −3.744 | −11.411 | −9.811 | 1.00 | 20.31 | C | C |
| ATOM | 9634 | SD | MET I | 370 | −5.284 | −10.974 | −10.642 | 1.00 | 26.43 | C | S |
| ATOM | 9635 | CE | MET I | 370 | −5.320 | −12.185 | −11.963 | 1.00 | 24.07 | C | C |
| ATOM | 9636 | C | MET I | 370 | −2.700 | −11.396 | −13.249 | 1.00 | 16.54 | C | C |
| ATOM | 9637 | O | MET I | 370 | −3.707 | −11.367 | −13.953 | 1.00 | 16.74 | C | O |
| ATOM | 9638 | N | ASN I | 371 | −1.610 | −10.691 | −13.540 | 1.00 | 16.35 | C | N |
| ATOM | 9639 | CA | ASN I | 371 | −1.614 | −9.772 | −14.680 | 1.00 | 16.07 | C | C |
| ATOM | 9640 | CB | ASN I | 371 | −1.375 | −8.326 | −14.232 | 1.00 | 16.41 | C | C |
| ATOM | 9641 | CG | ASN I | 371 | −2.619 | −7.690 | −13.634 | 1.00 | 16.71 | C | C |
| ATOM | 9642 | OD1 | ASN I | 371 | −3.501 | −7.228 | −14.356 | 1.00 | 15.75 | C | O |
| ATOM | 9643 | ND2 | ASN I | 371 | −2.722 | −7.724 | −12.310 | 1.00 | 17.76 | C | N |
| ATOM | 9644 | C | ASN I | 371 | −0.698 | −10.167 | −15.842 | 1.00 | 15.55 | C | C |
| ATOM | 9645 | O | ASN I | 371 | −0.650 | −9.478 | −16.862 | 1.00 | 15.84 | C | O |
| ATOM | 9646 | N | SER I | 372 | −0.166 | −11.383 | −15.771 | 1.00 | 14.76 | C | N |
| ATOM | 9647 | CA | SER I | 372 | 0.540 | −11.990 | −16.894 | 1.00 | 13.91 | C | C |
| ATOM | 9648 | CB | SER I | 372 | 0.561 | −13.512 | −16.746 | 1.00 | 14.04 | C | C |
| ATOM | 9649 | OG | SER I | 372 | −0.696 | −14.071 | −17.084 | 1.00 | 15.09 | C | O |
| ATOM | 9650 | C | SER I | 372 | −0.071 | −11.614 | −18.242 | 1.00 | 13.07 | C | C |
| ATOM | 9651 | O | SER I | 372 | −1.291 | −11.511 | −18.373 | 1.00 | 13.02 | C | O |
| ATOM | 9652 | N | LEU I | 373 | 0.781 | −11.525 | −19.263 | 1.00 | 12.23 | C | N |
| ATOM | 9653 | CA | LEU I | 373 | 0.352 | −11.621 | −20.658 | 1.00 | 11.45 | C | C |
| ATOM | 9654 | CB | LEU I | 373 | 0.999 | −10.504 | −21.485 | 1.00 | 11.43 | C | C |
| ATOM | 9655 | CG | LEU I | 373 | 0.250 | −9.176 | −21.636 | 1.00 | 11.04 | C | C |
| ATOM | 9656 | CD1 | LEU I | 373 | −0.580 | −8.867 | −20.406 | 1.00 | 11.76 | C | C |
| ATOM | 9657 | CD2 | LEU I | 373 | 1.213 | −8.035 | −21.928 | 1.00 | 10.49 | C | C |
| ATOM | 9658 | C | LEU I | 373 | 0.727 | −12.982 | −21.247 | 1.00 | 11.17 | C | C |
| ATOM | 9659 | O | LEU I | 373 | 1.819 | −13.492 | −21.000 | 1.00 | 11.27 | C | O |
| ATOM | 9660 | N | THR I | 374 | −0.164 | −13.546 | −22.056 | 1.00 | 10.70 | C | N |
| ATOM | 9661 | CA | THR I | 374 | 0.062 | −14.869 | −22.633 | 1.00 | 10.23 | C | C |
| ATOM | 9662 | CB | THR I | 374 | −1.187 | −15.768 | −22.531 | 1.00 | 10.24 | C | C |
| ATOM | 9663 | OG1 | THR I | 374 | −1.999 | −15.346 | −21.429 | 1.00 | 11.25 | C | O |
| ATOM | 9664 | CG2 | THR I | 374 | −0.785 | −17.224 | −22.338 | 1.00 | 10.52 | C | C |
| ATOM | 9665 | C | THR I | 374 | 0.481 | −14.772 | −24.091 | 1.00 | 9.54 | C | C |
| ATOM | 9666 | O | THR I | 374 | −0.164 | −14.097 | −24.893 | 1.00 | 9.70 | C | O |
| ATOM | 9667 | N | LEU I | 375 | 1.576 | −15.444 | −24.422 | 1.00 | 8.83 | C | N |
| ATOM | 9668 | CA | LEU I | 375 | 2.236 | −15.253 | −25.704 | 1.00 | 7.83 | C | C |
| ATOM | 9669 | CB | LEU I | 375 | 3.470 | −14.361 | −25.547 | 1.00 | 7.38 | C | C |
| ATOM | 9670 | CG | LEU I | 375 | 3.309 | −13.047 | −24.780 | 1.00 | 5.06 | C | C |
| ATOM | 9671 | CD1 | LEU I | 375 | 4.669 | −12.411 | −24.533 | 1.00 | 2.41 | C | C |
| ATOM | 9672 | CD2 | LEU I | 375 | 2.397 | −12.098 | −25.535 | 1.00 | 4.31 | C | C |
| ATOM | 9673 | C | LEU I | 375 | 2.644 | −16.600 | −26.283 | 1.00 | 7.79 | C | C |
| ATOM | 9674 | O | LEU I | 375 | 2.703 | −17.598 | −25.565 | 1.00 | 7.92 | C | O |
| ATOM | 9675 | N | PRO I | 376 | 2.914 | −16.629 | −27.595 | 1.00 | 7.51 | C | N |
| ATOM | 9676 | CA | PRO I | 376 | 3.325 | −17.826 | −28.323 | 1.00 | 7.90 | C | C |
| ATOM | 9677 | CB | PRO I | 376 | 2.852 | −17.538 | −29.748 | 1.00 | 7.90 | C | C |
| ATOM | 9678 | CG | PRO I | 376 | 2.913 | −16.062 | −29.858 | 1.00 | 7.97 | C | C |
| ATOM | 9679 | CD | PRO I | 376 | 2.537 | −15.534 | −28.505 | 1.00 | 7.40 | C | C |
| ATOM | 9680 | C | PRO I | 376 | 4.838 | −18.023 | −28.310 | 1.00 | 8.35 | C | C |
| ATOM | 9681 | O | PRO I | 376 | 5.589 | −17.062 | −28.484 | 1.00 | 8.71 | C | O |
| ATOM | 9682 | N | SER I | 377 | 5.263 | −19.283 | −28.263 | 1.00 | 9.11 | C | N |
| ATOM | 9683 | CA | SER I | 377 | 6.682 | −19.622 | −28.207 | 1.00 | 9.91 | C | C |
| ATOM | 9684 | CB | SER I | 377 | 6.880 | −21.137 | −28.330 | 1.00 | 10.23 | C | C |
| ATOM | 9685 | OG | SER I | 377 | 5.713 | −21.842 | −27.939 | 1.00 | 10.90 | C | O |
| ATOM | 9686 | C | SER I | 377 | 7.438 | −18.911 | −29.319 | 1.00 | 10.14 | C | C |
| ATOM | 9687 | O | SER I | 377 | 8.660 | −19.024 | −29.425 | 1.00 | 10.08 | C | O |
| ATOM | 9688 | N | GLU I | 378 | 6.683 | −18.286 | −30.215 | 1.00 | 10.71 | C | N |
| ATOM | 9689 | CA | GLU I | 378 | 7.256 | −17.563 | −31.339 | 1.00 | 11.40 | C | C |
| ATOM | 9690 | CB | GLU I | 378 | 6.177 | −17.256 | −32.379 | 1.00 | 11.65 | C | C |
| ATOM | 9691 | CG | GLU I | 378 | 5.858 | −18.418 | −33.312 | 1.00 | 13.46 | C | C |
| ATOM | 9692 | CD | GLU I | 378 | 5.249 | −19.608 | −32.588 | 1.00 | 16.20 | C | C |
| ATOM | 9693 | OE1 | GLU I | 378 | 4.999 | −19.505 | −31.368 | 1.00 | 15.73 | C | O |
| ATOM | 9694 | OE2 | GLU I | 378 | 4.989 | −20.636 | −33.250 | 1.00 | 18.38 | C | O |
| ATOM | 9695 | C | GLU I | 378 | 7.922 | −16.274 | −30.873 | 1.00 | 11.27 | C | C |
| ATOM | 9696 | O | GLU I | 378 | 8.776 | −15.722 | −31.567 | 1.00 | 11.28 | C | O |
| ATOM | 9697 | N | VAL I | 379 | 7.570 | −15.827 | −29.672 | 1.00 | 11.31 | C | N |
| ATOM | 9698 | CA | VAL I | 379 | 8.268 | −14.708 | −29.056 | 1.00 | 11.59 | C | C |
| ATOM | 9699 | CB | VAL I | 379 | 7.849 | −14.504 | −27.591 | 1.00 | 11.27 | C | C |
| ATOM | 9700 | CG1 | VAL I | 379 | 8.894 | −13.674 | −26.858 | 1.00 | 11.60 | C | C |
| ATOM | 9701 | CG2 | VAL I | 379 | 6.486 | −13.833 | −27.520 | 1.00 | 11.54 | C | C |
| ATOM | 9702 | C | VAL I | 379 | 9.779 | −14.907 | −29.124 | 1.00 | 12.16 | C | C |
| ATOM | 9703 | O | VAL I | 379 | 10.533 | −13.943 | −29.255 | 1.00 | 12.51 | C | O |
| ATOM | 9704 | N | ASN I | 380 | 10.219 | −16.158 | −29.030 | 1.00 | 12.98 | C | N |
| ATOM | 9705 | CA | ASN I | 380 | 11.637 | −16.453 | −28.854 | 1.00 | 14.00 | C | C |
| ATOM | 9706 | CB | ASN I | 380 | 11.854 | −17.947 | −28.579 | 1.00 | 14.59 | C | C |
| ATOM | 9707 | CG | ASN I | 380 | 11.318 | −18.381 | −27.224 | 1.00 | 17.09 | C | C |
| ATOM | 9708 | OD1 | ASN I | 380 | 12.070 | −18.488 | −26.256 | 1.00 | 20.41 | C | O |
| ATOM | 9709 | ND2 | ASN I | 380 | 10.045 | −18.763 | −27.186 | 1.00 | 18.86 | C | N |
| ATOM | 9710 | C | ASN I | 380 | 12.477 | −16.006 | −30.051 | 1.00 | 13.96 | C | C |
| ATOM | 9711 | O | ASN I | 380 | 13.619 | −15.576 | −29.890 | 1.00 | 14.38 | C | O |
| ATOM | 9712 | N | LEU I | 381 | 11.925 | −16.159 | −31.252 | 1.00 | 13.61 | C | N |
| ATOM | 9713 | CA | LEU I | 381 | 12.638 | −15.798 | −32.475 | 1.00 | 13.72 | C | C |
| ATOM | 9714 | CB | LEU I | 381 | 11.742 | −15.996 | −33.698 | 1.00 | 13.79 | C | C |
| ATOM | 9715 | CG | LEU I | 381 | 10.720 | −17.130 | −33.607 | 1.00 | 14.92 | C | C |
| ATOM | 9716 | CD1 | LEU I | 381 | 9.817 | −17.138 | −34.830 | 1.00 | 15.56 | C | C |
| ATOM | 9717 | CD2 | LEU I | 381 | 11.421 | −18.470 | −33.446 | 1.00 | 15.78 | C | C |
| ATOM | 9718 | C | LEU I | 381 | 13.093 | −14.349 | −32.401 | 1.00 | 13.62 | C | C |
| ATOM | 9719 | O | LEU I | 381 | 14.017 | −13.934 | −33.100 | 1.00 | 13.57 | C | O |
| ATOM | 9720 | N | CYS I | 382 | 12.473 | −13.607 | −31.494 | 1.00 | 13.47 | C | N |
| ATOM | 9721 | CA | CYS I | 382 | 12.727 | −12.187 | −31.357 | 1.00 | 13.57 | C | C |
| ATOM | 9722 | CB | CYS I | 382 | 11.613 | −11.538 | −30.545 | 1.00 | 13.62 | C | C |
| ATOM | 9723 | SG | CYS I | 382 | 10.346 | −10.770 | −31.551 | 1.00 | 15.64 | C | S |
| ATOM | 9724 | C | CYS I | 382 | 14.073 | −11.928 | −30.696 | 1.00 | 13.33 | C | C |
| ATOM | 9725 | O | CYS I | 382 | 14.690 | −10.887 | −30.923 | 1.00 | 13.57 | C | O |
| ATOM | 9726 | N | ASN I | 383 | 14.565 | −12.914 | −29.948 | 1.00 | 13.21 | C | N |
| ATOM | 9727 | CA | ASN I | 383 | 15.925 | −12.865 | −29.415 | 1.00 | 13.22 | C | C |
| ATOM | 9728 | CB | ASN I | 383 | 16.191 | −14.054 | −28.491 | 1.00 | 13.18 | C | C |
| ATOM | 9729 | CG | ASN I | 383 | 15.154 | −14.186 | −27.400 | 1.00 | 13.42 | C | C |
| ATOM | 9730 | OD1 | ASN I | 383 | 14.774 | −13.202 | −26.769 | 1.00 | 13.68 | C | O |
| ATOM | 9731 | ND2 | ASN I | 383 | 14.660 | −15.402 | −27.199 | 1.00 | 14.32 | C | N |
| ATOM | 9732 | C | ASN I | 383 | 16.959 | −12.845 | −30.530 | 1.00 | 13.28 | C | C |
| ATOM | 9733 | O | ASN I | 383 | 18.081 | −12.374 | −30.344 | 1.00 | 13.50 | C | O |
| ATOM | 9734 | N | VAL I | 384 | 16.605 | −13.451 | −31.657 | 1.00 | 13.24 | C | N |
| ATOM | 9735 | CA | VAL I | 384 | 17.532 | −13.584 | −32.767 | 1.00 | 13.29 | C | C |
| ATOM | 9736 | CB | VAL I | 384 | 17.484 | −14.992 | −33.387 | 1.00 | 13.28 | C | C |
| ATOM | 9737 | CG1 | VAL I | 384 | 18.046 | −14.970 | −34.800 | 1.00 | 13.73 | C | C |
| ATOM | 9738 | CG2 | VAL I | 384 | 18.251 | −15.977 | −32.518 | 1.00 | 12.76 | C | C |
| ATOM | 9739 | C | VAL I | 384 | 17.292 | −12.529 | −33.842 | 1.00 | 13.57 | C | C |
| ATOM | 9740 | O | VAL I | 384 | 18.239 | −12.070 | −34.480 | 1.00 | 13.31 | C | O |
| ATOM | 9741 | N | ASP I | 385 | 16.038 | −12.140 | −34.049 | 1.00 | 14.07 | C | N |
| ATOM | 9742 | CA | ASP I | 385 | 15.711 | −11.380 | −35.249 | 1.00 | 14.61 | C | C |
| ATOM | 9743 | CB | ASP I | 385 | 15.049 | −12.246 | −36.318 | 1.00 | 14.71 | C | C |
| ATOM | 9744 | CG | ASP I | 385 | 15.608 | −11.980 | −37.701 | 1.00 | 16.69 | C | C |
| ATOM | 9745 | OD1 | ASP I | 385 | 16.308 | −10.959 | −37.872 | 1.00 | 18.63 | C | O |
| ATOM | 9746 | OD2 | ASP I | 385 | 15.367 | −12.800 | −38.612 | 1.00 | 19.10 | C | O |
| ATOM | 9747 | C | ASP I | 385 | 15.014 | −10.029 | −35.092 | 1.00 | 14.63 | C | C |
| ATOM | 9748 | O | ASP I | 385 | 15.412 | −9.054 | −35.728 | 1.00 | 14.34 | C | O |
| ATOM | 9749 | N | ILE I | 386 | 13.937 | −9.980 | −34.314 | 1.00 | 14.54 | C | N |
| ATOM | 9750 | CA | ILE I | 386 | 13.154 | −8.752 | −34.212 | 1.00 | 14.46 | C | C |
| ATOM | 9751 | CB | ILE I | 386 | 14.074 | −7.521 | −34.137 | 1.00 | 13.83 | C | C |
| ATOM | 9752 | CG1 | ILE I | 386 | 14.932 | −7.584 | −32.875 | 1.00 | 13.44 | C | C |
| ATOM | 9753 | CD1 | ILE I | 386 | 16.315 | −7.018 | −33.048 | 1.00 | 12.39 | C | C |
| ATOM | 9754 | CG2 | ILE I | 386 | 13.253 | −6.241 | −34.161 | 1.00 | 13.51 | C | C |
| ATOM | 9755 | C | ILE I | 386 | 12.274 | −8.602 | −35.443 | 1.00 | 14.73 | C | C |
| ATOM | 9756 | O | ILE I | 386 | 11.048 | −8.692 | −35.369 | 1.00 | 14.68 | C | O |
| ATOM | 9757 | N | PHE I | 387 | 12.917 | −8.301 | −36.564 | 1.00 | 14.71 | C | N |
| ATOM | 9758 | CA | PHE I | 387 | 12.294 | −8.428 | −37.868 | 1.00 | 14.78 | C | C |
| ATOM | 9759 | CB | PHE I | 387 | 12.993 | −7.528 | −38.876 | 1.00 | 14.77 | C | C |
| ATOM | 9760 | CG | PHE I | 387 | 13.269 | −6.154 | −38.354 | 1.00 | 14.75 | C | C |
| ATOM | 9761 | CD1 | PHE I | 387 | 14.538 | −5.818 | −37.909 | 1.00 | 15.03 | C | C |
| ATOM | 9762 | CE1 | PHE I | 387 | 14.790 | −4.572 | −37.366 | 1.00 | 14.62 | C | C |
| ATOM | 9763 | CZ | PHE I | 387 | 13.743 | −3.702 | −37.131 | 1.00 | 14.55 | C | C |
| ATOM | 9764 | CE2 | PHE I | 387 | 12.452 | −4.074 | −37.463 | 1.00 | 14.68 | C | C |
| ATOM | 9765 | CD2 | PHE I | 387 | 12.219 | −5.303 | −38.052 | 1.00 | 14.61 | C | C |
| ATOM | 9766 | C | PHE I | 387 | 12.291 | −9.864 | −38.352 | 1.00 | 14.91 | C | C |
| ATOM | 9767 | O | PHE I | 387 | 13.339 | −10.461 | −38.604 | 1.00 | 14.86 | C | O |
| ATOM | 9768 | N | ASN I | 388 | 11.102 | −10.448 | −38.325 | 1.00 | 15.11 | C | N |
| ATOM | 9769 | CA | ASN I | 388 | 10.866 | −11.776 | −38.850 | 1.00 | 15.38 | C | C |
| ATOM | 9770 | CB | ASN I | 388 | 11.522 | −12.817 | −37.947 | 1.00 | 14.86 | C | C |
| ATOM | 9771 | CG | ASN I | 388 | 11.029 | −12.733 | −36.519 | 1.00 | 14.86 | C | C |
| ATOM | 9772 | OD1 | ASN I | 388 | 10.004 | −13.318 | −36.170 | 1.00 | 12.80 | C | O |
| ATOM | 9773 | ND2 | ASN I | 388 | 11.708 | −11.934 | −35.707 | 1.00 | 15.97 | C | N |
| ATOM | 9774 | C | ASN I | 388 | 9.363 | −11.982 | −38.843 | 1.00 | 16.11 | C | C |
| ATOM | 9775 | O | ASN I | 388 | 8.688 | −11.570 | −37.902 | 1.00 | 15.92 | C | O |
| ATOM | 9776 | N | PRO I | 389 | 8.817 | −12.450 | −39.969 | 1.00 | 16.99 | C | N |
| ATOM | 9777 | CA | PRO I | 389 | 7.464 | −12.988 | −39.920 | 1.00 | 17.40 | C | C |
| ATOM | 9778 | CB | PRO I | 389 | 7.376 | −13.815 | −41.204 | 1.00 | 17.72 | C | C |
| ATOM | 9779 | CG | PRO I | 389 | 8.776 | −14.223 | −41.477 | 1.00 | 17.47 | C | C |
| ATOM | 9780 | CD | PRO I | 389 | 9.618 | −13.063 | −41.043 | 1.00 | 17.05 | C | C |
| ATOM | 9781 | C | PRO I | 389 | 7.304 | −13.894 | −38.706 | 1.00 | 17.86 | C | C |
| ATOM | 9782 | O | PRO I | 389 | 8.297 | −14.397 | −38.181 | 1.00 | 18.45 | C | O |
| ATOM | 9783 | N | LYS I | 390 | 6.068 | −14.106 | −38.269 | 1.00 | 17.97 | C | N |
| ATOM | 9784 | CA | LYS I | 390 | 5.813 | −14.937 | −37.098 | 1.00 | 18.06 | C | C |
| ATOM | 9785 | CB | LYS I | 390 | 6.986 | −15.889 | −36.844 | 1.00 | 17.92 | C | C |
| ATOM | 9786 | CG | LYS I | 390 | 7.066 | −17.065 | −37.806 | 1.00 | 18.58 | C | C |
| ATOM | 9787 | CD | LYS I | 390 | 6.331 | −18.283 | −37.264 | 1.00 | 20.61 | C | C |
| ATOM | 9788 | CE | LYS I | 390 | 6.758 | −19.556 | −37.982 | 1.00 | 21.48 | C | C |
| ATOM | 9789 | NZ | LYS I | 390 | 6.197 | −19.645 | −39.360 | 1.00 | 23.16 | C | N |
| ATOM | 9790 | C | LYS I | 390 | 5.557 | −14.087 | −35.858 | 1.00 | 18.01 | C | C |
| ATOM | 9791 | O | LYS I | 390 | 4.552 | −14.267 | −35.171 | 1.00 | 18.43 | C | O |
| ATOM | 9792 | N | TYR I | 391 | 6.490 | −13.192 | −35.548 | 1.00 | 17.68 | C | N |
| ATOM | 9793 | CA | TYR I | 391 | 6.267 | −12.216 | −34.492 | 1.00 | 17.33 | C | C |
| ATOM | 9794 | CB | TYR I | 391 | 7.026 | −12.599 | −33.224 | 1.00 | 17.42 | C | C |
| ATOM | 9795 | CG | TYR I | 391 | 6.220 | −12.376 | −31.971 | 1.00 | 17.27 | C | C |
| ATOM | 9796 | CD1 | TYR I | 391 | 4.863 | −12.657 | −31.951 | 1.00 | 17.29 | C | C |
| ATOM | 9797 | CE1 | TYR I | 391 | 4.108 | −12.437 | −30.822 | 1.00 | 17.32 | C | C |
| ATOM | 9798 | CZ | TYR I | 391 | 4.678 | −11.809 | −29.739 | 1.00 | 16.51 | C | C |
| ATOM | 9799 | OH | TYR I | 391 | 3.934 | −11.601 | −28.602 | 1.00 | 16.65 | C | O |
| ATOM | 9800 | CE2 | TYR I | 391 | 6.005 | −11.441 | −29.765 | 1.00 | 16.52 | C | C |
| ATOM | 9801 | CD2 | TYR I | 391 | 6.756 | −11.689 | −30.893 | 1.00 | 17.24 | C | C |
| ATOM | 9802 | C | TYR I | 391 | 6.623 | −10.796 | −34.910 | 1.00 | 17.43 | C | C |
| ATOM | 9803 | O | TYR I | 391 | 7.786 | −10.491 | −35.175 | 1.00 | 17.70 | C | O |
| ATOM | 9804 | N | ASP I | 392 | 5.647 | −9.899 | −34.813 | 1.00 | 17.56 | C | N |
| ATOM | 9805 | CA | ASP I | 392 | 5.944 | −8.487 | −34.609 | 1.00 | 17.69 | C | C |
| ATOM | 9806 | CB | ASP I | 392 | 4.779 | −7.590 | −35.045 | 1.00 | 17.89 | C | C |
| ATOM | 9807 | CG | ASP I | 392 | 3.556 | −8.380 | −35.472 | 1.00 | 18.86 | C | C |
| ATOM | 9808 | OD1 | ASP I | 392 | 2.911 | −8.993 | −34.596 | 1.00 | 20.34 | C | O |
| ATOM | 9809 | OD2 | ASP I | 392 | 3.162 | −8.272 | −36.653 | 1.00 | 18.51 | C | O |
| ATOM | 9810 | C | ASP I | 392 | 6.342 | −8.188 | −33.169 | 1.00 | 17.11 | C | C |
| ATOM | 9811 | O | ASP I | 392 | 5.674 | −8.609 | −32.222 | 1.00 | 17.04 | C | O |
| ATOM | 9812 | N | CYS I | 393 | 7.356 | −7.342 | −33.026 | 1.00 | 16.33 | C | N |
| ATOM | 9813 | CA | CYS I | 393 | 8.347 | −7.491 | −31.975 | 1.00 | 16.17 | C | C |
| ATOM | 9814 | CB | CYS I | 393 | 9.656 | −8.022 | −32.554 | 1.00 | 16.11 | C | C |
| ATOM | 9815 | SG | CYS I | 393 | 10.738 | −8.792 | −31.342 | 1.00 | 20.23 | C | S |
| ATOM | 9816 | C | CYS I | 393 | 8.587 | −6.139 | −31.330 | 1.00 | 15.37 | C | C |
| ATOM | 9817 | O | CYS I | 393 | 9.066 | −5.209 | −31.980 | 1.00 | 15.15 | C | O |
| ATOM | 9818 | N | LYS I | 394 | 8.077 | −5.982 | −30.115 | 1.00 | 14.91 | C | N |
| ATOM | 9819 | CA | LYS I | 394 | 7.988 | −4.668 | −29.502 | 1.00 | 14.50 | C | C |
| ATOM | 9820 | CB | LYS I | 394 | 7.163 | −4.724 | −28.214 | 1.00 | 14.60 | C | C |
| ATOM | 9821 | CG | LYS I | 394 | 5.909 | −5.591 | −28.313 | 1.00 | 16.44 | C | C |
| ATOM | 9822 | CD | LYS I | 394 | 4.799 | −5.101 | −27.382 | 1.00 | 20.45 | C | C |
| ATOM | 9823 | CE | LYS I | 394 | 4.989 | −5.604 | −25.955 | 1.00 | 23.05 | C | C |
| ATOM | 9824 | NZ | LYS I | 394 | 5.685 | −4.608 | −25.094 | 1.00 | 24.13 | C | N |
| ATOM | 9825 | C | LYS I | 394 | 9.370 | −4.087 | −29.239 | 1.00 | 14.00 | C | C |
| ATOM | 9826 | O | LYS I | 394 | 10.260 | −4.760 | −28.720 | 1.00 | 14.14 | C | O |
| ATOM | 9827 | N | ILE I | 395 | 9.497 | −2.797 | −29.507 | 1.00 | 13.28 | C | N |
| ATOM | 9828 | CA | ILE I | 395 | 10.750 | −2.193 | −29.919 | 1.00 | 12.89 | C | C |
| ATOM | 9829 | CB | ILE I | 395 | 10.960 | −2.334 | −31.435 | 1.00 | 12.75 | C | C |
| ATOM | 9830 | CG1 | ILE I | 395 | 11.556 | −3.695 | −31.786 | 1.00 | 13.58 | C | C |
| ATOM | 9831 | CD1 | ILE I | 395 | 11.347 | −4.084 | −33.236 | 1.00 | 15.33 | C | C |
| ATOM | 9832 | CG2 | ILE I | 395 | 11.838 | −1.216 | −31.951 | 1.00 | 12.16 | C | C |
| ATOM | 9833 | C | ILE I | 395 | 10.534 | −0.722 | −29.648 | 1.00 | 13.15 | C | C |
| ATOM | 9834 | O | ILE I | 395 | 9.437 | −0.210 | −29.867 | 1.00 | 13.45 | C | O |
| ATOM | 9835 | N | MET I | 396 | 11.597 | −0.002 | −29.330 | 1.00 | 13.06 | C | N |
| ATOM | 9836 | CA | MET I | 396 | 11.526 | 1.433 | −29.454 | 1.00 | 13.00 | C | C |
| ATOM | 9837 | CB | MET I | 396 | 11.421 | 2.114 | −28.088 | 1.00 | 13.34 | C | C |
| ATOM | 9838 | CG | MET I | 396 | 12.738 | 2.594 | −27.506 | 1.00 | 14.30 | C | C |
| ATOM | 9839 | SD | MET I | 396 | 12.483 | 3.421 | −25.925 | 1.00 | 18.50 | C | S |
| ATOM | 9840 | CE | MET I | 396 | 11.067 | 2.515 | −25.304 | 1.00 | 17.80 | C | C |
| ATOM | 9841 | C | MET I | 396 | 12.646 | 2.009 | −30.288 | 1.00 | 12.96 | C | C |
| ATOM | 9842 | O | MET I | 396 | 13.704 | 1.405 | −30.453 | 1.00 | 13.04 | C | O |
| ATOM | 9843 | N | THR I | 397 | 12.360 | 3.167 | −30.862 | 1.00 | 12.78 | C | N |
| ATOM | 9844 | CA | THR I | 397 | 13.158 | 3.744 | −31.917 | 1.00 | 12.51 | C | C |
| ATOM | 9845 | CB | THR I | 397 | 12.261 | 4.048 | −33.134 | 1.00 | 12.39 | C | C |
| ATOM | 9846 | OG1 | THR I | 397 | 12.048 | 2.845 | −33.883 | 1.00 | 12.50 | C | O |
| ATOM | 9847 | CG2 | THR I | 397 | 12.895 | 5.107 | −34.026 | 1.00 | 12.55 | C | C |
| ATOM | 9848 | C | THR I | 397 | 13.633 | 5.062 | −31.330 | 1.00 | 12.58 | C | C |
| ATOM | 9849 | O | THR I | 397 | 12.815 | 5.911 | −31.001 | 1.00 | 12.88 | C | O |
| ATOM | 9850 | N | SER I | 398 | 14.919 | 5.217 | −31.088 | 1.00 | 12.55 | C | N |
| ATOM | 9851 | CA | SER I | 398 | 15.538 | 6.442 | −31.506 | 1.00 | 12.76 | C | C |
| ATOM | 9852 | CB | SER I | 398 | 16.509 | 6.920 | −30.432 | 1.00 | 12.52 | C | C |
| ATOM | 9853 | OG | SER I | 398 | 17.486 | 7.787 | −30.978 | 1.00 | 12.72 | C | O |
| ATOM | 9854 | C | SER I | 398 | 16.262 | 5.826 | −32.632 | 1.00 | 12.99 | C | C |
| ATOM | 9855 | O | SER I | 398 | 16.219 | 6.314 | −33.761 | 1.00 | 13.13 | C | O |
| ATOM | 9856 | N | LYS I | 399 | 16.463 | 4.540 | −32.418 | 1.00 | 13.01 | C | N |
| ATOM | 9857 | CA | LYS I | 399 | 17.619 | 4.000 | −32.989 | 1.00 | 13.29 | C | C |
| ATOM | 9858 | CB | LYS I | 399 | 17.475 | 4.221 | −34.500 | 1.00 | 13.79 | C | C |
| ATOM | 9859 | CG | LYS I | 399 | 15.997 | 4.359 | −34.942 | 1.00 | 14.34 | C | C |
| ATOM | 9860 | CD | LYS I | 399 | 15.854 | 4.556 | −36.445 | 1.00 | 16.43 | C | C |
| ATOM | 9861 | CE | LYS I | 399 | 15.114 | 3.394 | −37.093 | 1.00 | 17.37 | C | C |
| ATOM | 9862 | NZ | LYS I | 399 | 15.524 | 3.199 | −38.511 | 1.00 | 16.62 | C | N |
| ATOM | 9863 | C | LYS I | 399 | 18.632 | 4.961 | −32.349 | 1.00 | 13.09 | C | C |
| ATOM | 9864 | O | LYS I | 399 | 19.670 | 5.257 | −32.934 | 1.00 | 12.87 | C | O |
| ATOM | 9865 | N | THR I | 400 | 18.236 | 5.572 | −31.225 | 1.00 | 13.04 | C | N |
| ATOM | 9866 | CA | THR I | 400 | 19.072 | 6.571 | −30.513 | 1.00 | 13.38 | C | C |
| ATOM | 9867 | CB | THR I | 400 | 18.218 | 7.723 | −29.906 | 1.00 | 13.74 | C | C |
| ATOM | 9868 | OG1 | THR I | 400 | 16.861 | 7.632 | −30.365 | 1.00 | 14.38 | C | O |
| ATOM | 9869 | CG2 | THR I | 400 | 18.798 | 9.072 | −30.296 | 1.00 | 14.32 | C | C |
| ATOM | 9870 | C | THR I | 400 | 19.991 | 5.980 | −29.408 | 1.00 | 13.20 | C | C |
| ATOM | 9871 | O | THR I | 400 | 19.748 | 4.866 | −28.945 | 1.00 | 13.12 | C | O |
| ATOM | 9872 | N | ASP I | 401 | 20.881 | 6.801 | −28.834 | 1.00 | 12.97 | C | N |
| ATOM | 9873 | CA | ASP I | 401 | 22.197 | 6.333 | −28.331 | 1.00 | 12.50 | C | C |
| ATOM | 9874 | CB | ASP I | 401 | 23.307 | 7.270 | −28.816 | 1.00 | 12.73 | C | C |
| ATOM | 9875 | CG | ASP I | 401 | 24.212 | 6.623 | −29.841 | 1.00 | 13.98 | C | C |
| ATOM | 9876 | OD1 | ASP I | 401 | 23.677 | 6.029 | −30.801 | 1.00 | 16.55 | C | O |
| ATOM | 9877 | OD2 | ASP I | 401 | 25.430 | 6.903 | −29.814 | 1.00 | 14.80 | C | O |
| ATOM | 9878 | C | ASP I | 401 | 22.414 | 6.066 | −26.816 | 1.00 | 12.13 | C | C |
| ATOM | 9879 | O | ASP I | 401 | 21.541 | 6.376 | −26.002 | 1.00 | 12.31 | C | O |
| ATOM | 9880 | N | VAL I | 402 | 23.721 | 6.022 | −26.502 | 1.00 | 11.49 | C | N |
| ATOM | 9881 | CA | VAL I | 402 | 24.382 | 5.380 | −25.316 | 1.00 | 10.74 | C | C |
| ATOM | 9882 | CB | VAL I | 402 | 23.454 | 5.112 | −24.128 | 1.00 | 10.60 | C | C |
| ATOM | 9883 | CG1 | VAL I | 402 | 24.200 | 4.335 | −23.060 | 1.00 | 11.26 | C | C |
| ATOM | 9884 | CG2 | VAL I | 402 | 22.942 | 6.425 | −23.556 | 1.00 | 10.99 | C | C |
| ATOM | 9885 | C | VAL I | 402 | 25.452 | 4.251 | −25.504 | 1.00 | 10.24 | C | C |
| ATOM | 9886 | O | VAL I | 402 | 25.773 | 3.895 | −26.638 | 1.00 | 10.30 | C | O |
| ATOM | 9887 | N | SER I | 403 | 25.962 | 3.665 | −24.400 | 1.00 | 9.76 | C | N |
| ATOM | 9888 | CA | SER I | 403 | 27.019 | 2.595 | −24.456 | 1.00 | 9.18 | C | C |
| ATOM | 9889 | CB | SER I | 403 | 28.324 | 3.145 | −25.036 | 1.00 | 9.41 | C | C |
| ATOM | 9890 | OG | SER I | 403 | 28.274 | 4.554 | −25.165 | 1.00 | 10.82 | C | O |
| ATOM | 9891 | C | SER I | 403 | 27.325 | 1.652 | −23.238 | 1.00 | 8.51 | C | C |
| ATOM | 9892 | O | SER I | 403 | 27.347 | 2.099 | −22.088 | 1.00 | 8.30 | C | O |
| ATOM | 9893 | N | SER I | 404 | 27.642 | 0.376 | −23.530 | 1.00 | 8.35 | C | N |
| ATOM | 9894 | CA | SER I | 404 | 28.574 | −0.482 | −22.733 | 1.00 | 8.18 | C | C |
| ATOM | 9895 | CB | SER I | 404 | 28.123 | −0.585 | −21.276 | 1.00 | 8.18 | C | C |
| ATOM | 9896 | OG | SER I | 404 | 27.698 | −1.901 | −20.968 | 1.00 | 8.83 | C | O |
| ATOM | 9897 | C | SER I | 404 | 28.789 | −1.907 | −23.310 | 1.00 | 7.62 | C | C |
| ATOM | 9898 | O | SER I | 404 | 28.422 | −2.171 | −24.455 | 1.00 | 7.57 | C | O |
| ATOM | 9899 | N | SER I | 405 | 29.368 | −2.817 | −22.514 | 1.00 | 7.08 | C | N |
| ATOM | 9900 | CA | SER I | 405 | 29.672 | −4.192 | −22.973 | 1.00 | 6.53 | C | C |
| ATOM | 9901 | CB | SER I | 405 | 31.139 | −4.335 | −23.400 | 1.00 | 6.42 | C | C |
| ATOM | 9902 | OG | SER I | 405 | 31.467 | −5.695 | −23.651 | 1.00 | 5.66 | C | O |
| ATOM | 9903 | C | SER I | 405 | 29.300 | −5.333 | −22.012 | 1.00 | 6.28 | C | C |
| ATOM | 9904 | O | SER I | 405 | 29.334 | −5.169 | −20.791 | 1.00 | 5.98 | C | O |
| ATOM | 9905 | N | VAL I | 406 | 29.215 | −6.535 | −22.576 | 1.00 | 6.22 | C | N |
| ATOM | 9906 | CA | VAL I | 406 | 28.714 | −7.705 | −21.866 | 1.00 | 6.50 | C | C |
| ATOM | 9907 | CB | VAL I | 406 | 27.208 | −7.894 | −22.099 | 1.00 | 6.45 | C | C |
| ATOM | 9908 | CG1 | VAL I | 406 | 26.661 | −8.972 | −21.181 | 1.00 | 7.66 | C | C |
| ATOM | 9909 | CG2 | VAL I | 406 | 26.470 | −6.577 | −21.903 | 1.00 | 7.38 | C | C |
| ATOM | 9910 | C | VAL I | 406 | 29.424 | −8.942 | −22.388 | 1.00 | 6.24 | C | C |
| ATOM | 9911 | O | VAL I | 406 | 29.397 | −9.228 | −23.584 | 1.00 | 6.14 | C | O |
| ATOM | 9912 | N | ILE I | 407 | 30.041 | −9.689 | −21.485 | 1.00 | 6.32 | C | N |
| ATOM | 9913 | CA | ILE I | 407 | 30.831 | −10.838 | −21.881 | 1.00 | 6.60 | C | C |
| ATOM | 9914 | CB | ILE I | 407 | 32.035 | −11.021 | −20.959 | 1.00 | 6.27 | C | C |
| ATOM | 9915 | CG1 | ILE I | 407 | 33.112 | −9.989 | −21.295 | 1.00 | 6.19 | C | C |
| ATOM | 9916 | CD1 | ILE I | 407 | 33.720 | −9.323 | −20.073 | 1.00 | 8.34 | C | C |
| ATOM | 9917 | CG2 | ILE I | 407 | 32.567 | −12.438 | −21.053 | 1.00 | 6.42 | C | C |
| ATOM | 9918 | C | ILE I | 407 | 29.995 | −12.110 | −21.882 | 1.00 | 7.20 | C | C |
| ATOM | 9919 | O | ILE I | 407 | 29.246 | −12.376 | −20.944 | 1.00 | 7.42 | C | O |
| ATOM | 9920 | N | THR I | 408 | 30.163 | −12.914 | −22.925 | 1.00 | 7.57 | C | N |
| ATOM | 9921 | CA | THR I | 408 | 29.355 | −14.113 | −23.103 | 1.00 | 8.19 | C | C |
| ATOM | 9922 | CB | THR I | 408 | 28.681 | −14.138 | −24.483 | 1.00 | 8.15 | C | C |
| ATOM | 9923 | OG1 | THR I | 408 | 29.684 | −14.218 | −25.503 | 1.00 | 8.88 | C | O |
| ATOM | 9924 | CG2 | THR I | 408 | 27.853 | −12.888 | −24.693 | 1.00 | 7.93 | C | C |
| ATOM | 9925 | C | THR I | 408 | 30.192 | −15.376 | −22.966 | 1.00 | 8.55 | C | C |
| ATOM | 9926 | O | THR I | 408 | 31.419 | −15.333 | −23.065 | 1.00 | 9.07 | C | O |
| ATOM | 9927 | N | SER I | 409 | 29.503 | −16.512 | −22.970 | 1.00 | 8.70 | C | N |
| ATOM | 9928 | CA | SER I | 409 | 30.146 | −17.813 | −22.838 | 1.00 | 9.20 | C | C |
| ATOM | 9929 | CB | SER I | 409 | 29.126 | −18.935 | −23.044 | 1.00 | 9.72 | C | C |
| ATOM | 9930 | OG | SER I | 409 | 27.946 | −18.700 | −22.297 | 1.00 | 10.75 | C | O |
| ATOM | 9931 | C | SER I | 409 | 31.268 | −17.958 | −23.848 | 1.00 | 9.01 | C | C |
| ATOM | 9932 | O | SER I | 409 | 32.299 | −18.571 | −23.567 | 1.00 | 9.02 | C | O |
| ATOM | 9933 | N | LEU I | 410 | 31.027 | −17.458 | −25.053 | 1.00 | 8.91 | C | N |
| ATOM | 9934 | CA | LEU I | 410 | 31.814 | −17.858 | −26.204 | 1.00 | 9.29 | C | C |
| ATOM | 9935 | CB | LEU I | 410 | 31.024 | −18.828 | −27.081 | 1.00 | 9.61 | C | C |
| ATOM | 9936 | CG | LEU I | 410 | 31.702 | −20.180 | −27.323 | 1.00 | 9.70 | C | C |
| ATOM | 9937 | CD1 | LEU I | 410 | 31.975 | −20.908 | −26.014 | 1.00 | 11.11 | C | C |
| ATOM | 9938 | CD2 | LEU I | 410 | 30.895 | −21.057 | −28.274 | 1.00 | 9.16 | C | C |
| ATOM | 9939 | C | LEU I | 410 | 32.250 | −16.644 | −27.009 | 1.00 | 9.12 | C | C |
| ATOM | 9940 | O | LEU I | 410 | 32.844 | −16.774 | −28.077 | 1.00 | 9.08 | C | O |
| ATOM | 9941 | N | GLY I | 411 | 32.016 | −15.462 | −26.454 | 1.00 | 9.03 | C | N |
| ATOM | 9942 | CA | GLY I | 411 | 32.612 | −14.247 | −26.984 | 1.00 | 8.44 | C | C |
| ATOM | 9943 | C | GLY I | 411 | 32.234 | −13.047 | −26.146 | 1.00 | 7.91 | C | C |
| ATOM | 9944 | O | GLY I | 411 | 32.175 | −13.131 | −24.921 | 1.00 | 7.85 | C | O |
| ATOM | 9945 | N | ALA I | 412 | 31.868 | −11.961 | −26.816 | 1.00 | 7.30 | C | N |
| ATOM | 9946 | CA | ALA I | 412 | 31.416 | −10.757 | −26.130 | 1.00 | 6.92 | C | C |
| ATOM | 9947 | CB | ALA I | 412 | 32.609 | −9.925 | −25.672 | 1.00 | 6.90 | C | C |
| ATOM | 9948 | C | ALA I | 412 | 30.502 | −9.934 | −27.029 | 1.00 | 6.99 | C | C |
| ATOM | 9949 | O | ALA I | 412 | 30.597 | −10.004 | −28.255 | 1.00 | 6.94 | C | O |
| ATOM | 9950 | N | ILE I | 413 | 29.567 | −9.216 | −26.418 | 1.00 | 7.29 | C | N |
| ATOM | 9951 | CA | ILE I | 413 | 28.808 | −8.200 | −27.136 | 1.00 | 7.49 | C | C |
| ATOM | 9952 | CB | ILE I | 413 | 27.296 | −8.285 | −26.846 | 1.00 | 7.24 | C | C |
| ATOM | 9953 | CG1 | ILE I | 413 | 26.703 | −9.550 | −27.480 | 1.00 | 7.18 | C | C |
| ATOM | 9954 | CD1 | ILE I | 413 | 25.397 | −9.315 | −28.241 | 1.00 | 7.70 | C | C |
| ATOM | 9955 | CG2 | ILE I | 413 | 26.585 | −7.051 | −27.397 | 1.00 | 7.70 | C | C |
| ATOM | 9956 | C | ILE I | 413 | 29.304 | −6.811 | −26.776 | 1.00 | 7.94 | C | C |
| ATOM | 9957 | O | ILE I | 413 | 29.806 | −6.591 | −25.674 | 1.00 | 8.39 | C | O |
| ATOM | 9958 | N | VAL I | 414 | 29.155 | −5.871 | −27.703 | 1.00 | 8.22 | C | N |
| ATOM | 9959 | CA | VAL I | 414 | 29.498 | −4.485 | −27.420 | 1.00 | 8.77 | C | C |
| ATOM | 9960 | CB | VAL I | 414 | 30.965 | −4.158 | −27.782 | 1.00 | 8.57 | C | C |
| ATOM | 9961 | CG1 | VAL I | 414 | 31.293 | −4.627 | −29.197 | 1.00 | 9.12 | C | C |
| ATOM | 9962 | CG2 | VAL I | 414 | 31.245 | −2.669 | −27.607 | 1.00 | 8.90 | C | C |
| ATOM | 9963 | C | VAL I | 414 | 28.536 | −3.470 | −28.028 | 1.00 | 8.92 | C | C |
| ATOM | 9964 | O | VAL I | 414 | 28.246 | −3.497 | −29.227 | 1.00 | 9.00 | C | O |
| ATOM | 9965 | N | SER I | 415 | 27.947 | −2.665 | −27.152 | 1.00 | 8.97 | C | N |
| ATOM | 9966 | CA | SER I | 415 | 27.023 | −1.623 | −27.552 | 1.00 | 9.02 | C | C |
| ATOM | 9967 | CB | SER I | 415 | 25.805 | −1.623 | −26.629 | 1.00 | 8.98 | C | C |
| ATOM | 9968 | OG | SER I | 415 | 25.203 | −2.903 | −26.581 | 1.00 | 9.54 | C | O |
| ATOM | 9969 | C | SER I | 415 | 27.727 | −0.277 | −27.480 | 1.00 | 9.36 | C | C |
| ATOM | 9970 | O | SER I | 415 | 27.996 | 0.230 | −26.392 | 1.00 | 9.61 | C | O |
| ATOM | 9971 | N | CYS I | 416 | 28.219 | 0.177 | −28.626 | 1.00 | 9.57 | C | N |
| ATOM | 9972 | CA | CYS I | 416 | 28.920 | 1.447 | −28.701 | 1.00 | 9.61 | C | C |
| ATOM | 9973 | CB | CYS I | 416 | 30.167 | 1.320 | −29.572 | 1.00 | 9.62 | C | C |
| ATOM | 9974 | SG | CYS I | 416 | 31.508 | 2.403 | −29.060 | 1.00 | 11.33 | C | S |
| ATOM | 9975 | C | CYS I | 416 | 28.011 | 2.519 | −29.270 | 1.00 | 9.61 | C | C |
| ATOM | 9976 | O | CYS I | 416 | 27.619 | 2.458 | −30.434 | 1.00 | 9.80 | C | O |
| ATOM | 9977 | N | TYR I | 417 | 27.662 | 3.493 | −28.441 | 1.00 | 9.48 | C | N |
| ATOM | 9978 | CA | TYR I | 417 | 26.667 | 4.475 | −28.829 | 1.00 | 9.28 | C | C |
| ATOM | 9979 | CB | TYR I | 417 | 25.305 | 4.117 | −28.235 | 1.00 | 9.17 | C | C |
| ATOM | 9980 | CG | TYR I | 417 | 24.629 | 2.955 | −28.924 | 1.00 | 8.71 | C | C |
| ATOM | 9981 | CD1 | TYR I | 417 | 23.902 | 2.024 | −28.199 | 1.00 | 8.54 | C | C |
| ATOM | 9982 | CE1 | TYR I | 417 | 23.228 | 0.999 | −28.830 | 1.00 | 9.56 | C | C |
| ATOM | 9983 | CZ | TYR I | 417 | 23.289 | 0.886 | −30.201 | 1.00 | 9.83 | C | C |
| ATOM | 9984 | OH | TYR I | 417 | 22.658 | −0.162 | −30.829 | 1.00 | 10.51 | C | O |
| ATOM | 9985 | CE2 | TYR I | 417 | 24.025 | 1.783 | −30.942 | 1.00 | 9.36 | C | C |
| ATOM | 9986 | CD2 | TYR I | 417 | 24.675 | 2.818 | −30.305 | 1.00 | 8.95 | C | C |
| ATOM | 9987 | C | TYR I | 417 | 27.076 | 5.885 | −28.422 | 1.00 | 9.35 | C | C |
| ATOM | 9988 | O | TYR I | 417 | 28.046 | 6.075 | −27.687 | 1.00 | 9.11 | C | O |
| ATOM | 9989 | N | GLY I | 418 | 26.357 | 6.872 | −28.946 | 1.00 | 9.71 | C | N |
| ATOM | 9990 | CA | GLY I | 418 | 26.662 | 8.270 | −28.679 | 1.00 | 9.95 | C | C |
| ATOM | 9991 | C | GLY I | 418 | 28.094 | 8.602 | −29.035 | 1.00 | 10.09 | C | C |
| ATOM | 9992 | O | GLY I | 418 | 28.623 | 8.112 | −30.033 | 1.00 | 10.17 | C | O |
| ATOM | 9993 | N | LYS I | 419 | 28.768 | 9.307 | −28.136 | 1.00 | 10.17 | C | N |
| ATOM | 9994 | CA | LYS I | 419 | 30.104 | 9.810 | −28.415 | 1.00 | 10.50 | C | C |
| ATOM | 9995 | CB | LYS I | 419 | 30.381 | 11.077 | −27.607 | 1.00 | 11.09 | C | C |
| ATOM | 9996 | CG | LYS I | 419 | 29.942 | 12.351 | −28.300 | 1.00 | 12.94 | C | C |
| ATOM | 9997 | CD | LYS I | 419 | 30.697 | 13.553 | −27.770 | 1.00 | 15.52 | C | C |
| ATOM | 9998 | CE | LYS I | 419 | 30.546 | 14.735 | −28.708 | 1.00 | 17.07 | C | C |
| ATOM | 9999 | NZ | LYS I | 419 | 29.200 | 14.756 | −29.349 | 1.00 | 19.30 | C | N |
| ATOM | 10000 | C | LYS I | 419 | 31.201 | 8.777 | −28.168 | 1.00 | 10.23 | C | C |
| ATOM | 10001 | O | LYS I | 419 | 32.380 | 9.063 | −28.374 | 1.00 | 10.58 | C | O |
| ATOM | 10002 | N | THR I | 420 | 30.823 | 7.592 | −27.697 | 1.00 | 10.10 | C | N |
| ATOM | 10003 | CA | THR I | 420 | 31.789 | 6.687 | −27.076 | 1.00 | 9.74 | C | C |
| ATOM | 10004 | CB | THR I | 420 | 31.106 | 5.656 | −26.158 | 1.00 | 9.28 | C | C |
| ATOM | 10005 | OG1 | THR I | 420 | 29.766 | 6.076 | −25.878 | 1.00 | 9.21 | C | O |
| ATOM | 10006 | CG2 | THR I | 420 | 31.870 | 5.521 | −24.851 | 1.00 | 9.03 | C | C |
| ATOM | 10007 | C | THR I | 420 | 32.671 | 5.966 | −28.096 | 1.00 | 9.95 | C | C |
| ATOM | 10008 | O | THR I | 420 | 32.289 | 5.800 | −29.254 | 1.00 | 10.35 | C | O |
| ATOM | 10009 | N | LYS I | 421 | 33.880 | 5.608 | −27.674 | 1.00 | 10.07 | C | N |
| ATOM | 10010 | CA | LYS I | 421 | 34.808 | 4.863 | −28.521 | 1.00 | 10.21 | C | C |
| ATOM | 10011 | CB | LYS I | 421 | 36.194 | 5.513 | −28.495 | 1.00 | 10.30 | C | C |
| ATOM | 10012 | CG | LYS I | 421 | 36.445 | 6.496 | −29.626 | 1.00 | 12.43 | C | C |
| ATOM | 10013 | CD | LYS I | 421 | 37.766 | 7.225 | −29.441 | 1.00 | 15.12 | C | C |
| ATOM | 10014 | CE | LYS I | 421 | 37.752 | 8.578 | −30.134 | 1.00 | 15.21 | C | C |
| ATOM | 10015 | NZ | LYS I | 421 | 38.779 | 9.500 | −29.575 | 1.00 | 15.98 | C | N |
| ATOM | 10016 | C | LYS I | 421 | 34.908 | 3.412 | −28.063 | 1.00 | 10.09 | C | C |
| ATOM | 10017 | O | LYS I | 421 | 34.979 | 3.137 | −26.866 | 1.00 | 10.36 | C | O |
| ATOM | 10018 | N | CYS I | 422 | 34.937 | 2.486 | −29.017 | 1.00 | 9.81 | C | N |
| ATOM | 10019 | CA | CYS I | 422 | 35.063 | 1.075 | −28.681 | 1.00 | 9.56 | C | C |
| ATOM | 10020 | CB | CYS I | 422 | 33.696 | 0.396 | −28.633 | 1.00 | 9.72 | C | C |
| ATOM | 10021 | SG | CYS I | 422 | 32.479 | 1.300 | −27.664 | 1.00 | 11.42 | C | S |
| ATOM | 10022 | C | CYS I | 422 | 36.012 | 0.311 | −29.590 | 1.00 | 9.18 | C | C |
| ATOM | 10023 | O | CYS I | 422 | 36.111 | 0.582 | −30.787 | 1.00 | 9.27 | C | O |
| ATOM | 10024 | N | THR I | 423 | 36.636 | −0.710 | −29.016 | 1.00 | 8.93 | C | N |
| ATOM | 10025 | CA | THR I | 423 | 37.825 | −1.322 | −29.583 | 1.00 | 8.64 | C | C |
| ATOM | 10026 | CB | THR I | 423 | 39.104 | −0.757 | −28.940 | 1.00 | 8.32 | C | C |
| ATOM | 10027 | OG1 | THR I | 423 | 39.059 | 0.675 | −28.954 | 1.00 | 7.97 | C | O |
| ATOM | 10028 | CG2 | THR I | 423 | 40.336 | −1.230 | −29.695 | 1.00 | 8.36 | C | C |
| ATOM | 10029 | C | THR I | 423 | 37.771 | −2.810 | −29.287 | 1.00 | 8.86 | C | C |
| ATOM | 10030 | O | THR I | 423 | 37.514 | −3.214 | −28.153 | 1.00 | 9.26 | C | O |
| ATOM | 10031 | N | ALA I | 424 | 37.993 | −3.625 | −30.310 | 1.00 | 8.74 | C | N |
| ATOM | 10032 | CA | ALA I | 424 | 38.356 | −5.018 | −30.097 | 1.00 | 8.79 | C | C |
| ATOM | 10033 | CB | ALA I | 424 | 37.512 | −5.927 | −30.977 | 1.00 | 8.94 | C | C |
| ATOM | 10034 | C | ALA I | 424 | 39.839 | −5.233 | −30.371 | 1.00 | 8.84 | C | C |
| ATOM | 10035 | O | ALA I | 424 | 40.316 | −4.985 | −31.477 | 1.00 | 9.24 | C | O |
| ATOM | 10036 | N | SER I | 425 | 40.570 | −5.661 | −29.347 | 1.00 | 8.81 | C | N |
| ATOM | 10037 | CA | SER I | 425 | 41.990 | −5.958 | −29.492 | 1.00 | 8.85 | C | C |
| ATOM | 10038 | CB | SER I | 425 | 42.803 | −5.192 | −28.446 | 1.00 | 8.94 | C | C |
| ATOM | 10039 | OG | SER I | 425 | 42.091 | −4.060 | −27.976 | 1.00 | 7.69 | C | O |
| ATOM | 10040 | C | SER I | 425 | 42.253 | −7.456 | −29.368 | 1.00 | 9.01 | C | C |
| ATOM | 10041 | O | SER I | 425 | 41.541 | −8.163 | −28.656 | 1.00 | 8.63 | C | O |
| ATOM | 10042 | N | ASN I | 426 | 43.246 | −7.944 | −30.102 | 1.00 | 9.54 | C | N |
| ATOM | 10043 | CA | ASN I | 426 | 43.519 | −9.374 | −30.153 | 1.00 | 10.19 | C | C |
| ATOM | 10044 | CB | ASN I | 426 | 44.105 | −9.764 | −31.511 | 1.00 | 9.77 | C | C |
| ATOM | 10045 | CG | ASN I | 426 | 45.597 | −9.506 | −31.599 | 1.00 | 9.43 | C | C |
| ATOM | 10046 | OD1 | ASN I | 426 | 46.249 | −9.211 | −30.597 | 1.00 | 9.01 | C | O |
| ATOM | 10047 | ND2 | ASN I | 426 | 46.146 | −9.616 | −32.803 | 1.00 | 9.01 | C | N |
| ATOM | 10048 | C | ASN I | 426 | 44.434 | −9.853 | −29.029 | 1.00 | 11.16 | C | C |
| ATOM | 10049 | O | ASN I | 426 | 44.674 | −9.138 | −28.056 | 1.00 | 11.06 | C | O |
| ATOM | 10050 | N | LYS I | 427 | 44.921 | −11.083 | −29.170 | 1.00 | 12.38 | C | N |
| ATOM | 10051 | CA | LYS I | 427 | 45.803 | −11.703 | −28.185 | 1.00 | 13.52 | C | C |
| ATOM | 10052 | CB | LYS I | 427 | 46.418 | −12.978 | −28.765 | 1.00 | 14.00 | C | C |
| ATOM | 10053 | CG | LYS I | 427 | 45.683 | −14.255 | −28.398 | 1.00 | 16.18 | C | C |
| ATOM | 10054 | CD | LYS I | 427 | 46.510 | −15.478 | −28.758 | 1.00 | 18.24 | C | C |
| ATOM | 10055 | CE | LYS I | 427 | 46.211 | −16.641 | −27.828 | 1.00 | 19.72 | C | C |
| ATOM | 10056 | NZ | LYS I | 427 | 46.794 | −16.434 | −26.474 | 1.00 | 21.43 | C | N |
| ATOM | 10057 | C | LYS I | 427 | 46.915 | −10.758 | −27.741 | 1.00 | 13.71 | C | C |
| ATOM | 10058 | O | LYS I | 427 | 47.180 | −10.611 | −26.547 | 1.00 | 13.63 | C | O |
| ATOM | 10059 | N | ASN I | 428 | 47.634 | −10.210 | −28.716 | 1.00 | 14.28 | C | N |
| ATOM | 10060 | CA | ASN I | 428 | 48.816 | −9.400 | −28.447 | 1.00 | 14.60 | C | C |
| ATOM | 10061 | CB | ASN I | 428 | 49.709 | −9.331 | −29.689 | 1.00 | 14.57 | C | C |
| ATOM | 10062 | CG | ASN I | 428 | 49.589 | −10.565 | −30.563 | 1.00 | 13.24 | C | C |
| ATOM | 10063 | OD1 | ASN I | 428 | 49.765 | −11.690 | −30.096 | 1.00 | 9.74 | C | O |
| ATOM | 10064 | ND2 | ASN I | 428 | 49.319 | −10.358 | −31.847 | 1.00 | 12.83 | C | N |
| ATOM | 10065 | C | ASN I | 428 | 48.466 | −7.989 | −27.982 | 1.00 | 14.83 | C | C |
| ATOM | 10066 | O | ASN I | 428 | 49.330 | −7.255 | −27.499 | 1.00 | 15.25 | C | O |
| ATOM | 10067 | N | ARG I | 429 | 47.225 | −7.580 | −28.226 | 1.00 | 14.78 | C | N |
| ATOM | 10068 | CA | ARG I | 429 | 46.775 | −6.234 | −27.885 | 1.00 | 14.74 | C | C |
| ATOM | 10069 | CB | ARG I | 429 | 47.695 | −5.603 | −26.837 | 1.00 | 14.93 | C | C |
| ATOM | 10070 | CG | ARG I | 429 | 47.475 | −6.121 | −25.424 | 1.00 | 16.26 | C | C |
| ATOM | 10071 | CD | ARG I | 429 | 46.452 | −5.279 | −24.677 | 1.00 | 19.79 | C | C |
| ATOM | 10072 | NE | ARG I | 429 | 45.979 | −5.940 | −23.464 | 1.00 | 22.68 | C | N |
| ATOM | 10073 | CZ | ARG I | 429 | 46.735 | −6.172 | −22.396 | 1.00 | 23.54 | C | C |
| ATOM | 10074 | NH1 | ARG I | 429 | 48.013 | −5.817 | −22.394 | 1.00 | 22.87 | C | N |
| ATOM | 10075 | NH2 | ARG I | 429 | 46.219 | −6.777 | −21.335 | 1.00 | 23.53 | C | N |
| ATOM | 10076 | C | ARG I | 429 | 46.689 | −5.339 | −29.117 | 1.00 | 14.35 | C | C |
| ATOM | 10077 | O | ARG I | 429 | 46.093 | −4.262 | −29.073 | 1.00 | 14.62 | C | O |
| ATOM | 10078 | N | GLY I | 430 | 47.270 | −5.798 | −30.221 | 1.00 | 13.67 | C | N |
| ATOM | 10079 | CA | GLY I | 430 | 46.898 | −5.304 | −31.540 | 1.00 | 12.80 | C | C |
| ATOM | 10080 | C | GLY I | 430 | 45.397 | −5.149 | −31.683 | 1.00 | 12.11 | C | C |
| ATOM | 10081 | O | GLY I | 430 | 44.634 | −6.038 | −31.306 | 1.00 | 12.18 | C | O |
| ATOM | 10082 | N | ILE I | 431 | 44.972 | −4.001 | −32.199 | 1.00 | 11.58 | C | N |
| ATOM | 10083 | CA | ILE I | 431 | 43.552 | −3.678 | −32.300 | 1.00 | 10.76 | C | C |
| ATOM | 10084 | CB | ILE I | 431 | 43.321 | −2.162 | −32.138 | 1.00 | 10.43 | C | C |
| ATOM | 10085 | CG1 | ILE I | 431 | 43.649 | −1.718 | −30.711 | 1.00 | 10.95 | C | C |
| ATOM | 10086 | CD1 | ILE I | 431 | 44.264 | −0.337 | −30.628 | 1.00 | 12.08 | C | C |
| ATOM | 10087 | CG2 | ILE I | 431 | 41.892 | −1.796 | −32.507 | 1.00 | 10.07 | C | C |
| ATOM | 10088 | C | ILE I | 431 | 43.011 | −4.108 | −33.660 | 1.00 | 10.68 | C | C |
| ATOM | 10089 | O | ILE I | 431 | 43.474 | −3.623 | −34.693 | 1.00 | 10.40 | C | O |
| ATOM | 10090 | N | ILE I | 432 | 42.063 | −5.041 | −33.669 | 1.00 | 10.90 | C | N |
| ATOM | 10091 | CA | ILE I | 432 | 41.497 | −5.518 | −34.931 | 1.00 | 11.42 | C | C |
| ATOM | 10092 | CB | ILE I | 432 | 41.106 | −7.008 | −34.884 | 1.00 | 11.63 | C | C |
| ATOM | 10093 | CG1 | ILE I | 432 | 41.784 | −7.712 | −33.709 | 1.00 | 12.68 | C | C |
| ATOM | 10094 | CD1 | ILE I | 432 | 41.172 | −9.055 | −33.369 | 1.00 | 13.89 | C | C |
| ATOM | 10095 | CG2 | ILE I | 432 | 41.453 | −7.689 | −36.201 | 1.00 | 12.24 | C | C |
| ATOM | 10096 | C | ILE I | 432 | 40.290 | −4.703 | −35.381 | 1.00 | 11.41 | C | C |
| ATOM | 10097 | O | ILE I | 432 | 40.172 | −4.362 | −36.558 | 1.00 | 11.63 | C | O |
| ATOM | 10098 | N | LYS I | 433 | 39.315 | −4.555 | −34.492 | 1.00 | 11.52 | C | N |
| ATOM | 10099 | CA | LYS I | 433 | 38.076 | −3.878 | −34.848 | 1.00 | 11.96 | C | C |
| ATOM | 10100 | CB | LYS I | 433 | 36.864 | −4.767 | −34.566 | 1.00 | 12.31 | C | C |
| ATOM | 10101 | CG | LYS I | 433 | 35.552 | −4.201 | −35.092 | 1.00 | 14.42 | C | C |
| ATOM | 10102 | CD | LYS I | 433 | 34.622 | −5.301 | −35.578 | 1.00 | 16.74 | C | C |
| ATOM | 10103 | CE | LYS I | 433 | 33.367 | −4.723 | −36.216 | 1.00 | 17.08 | C | C |
| ATOM | 10104 | NZ | LYS I | 433 | 33.135 | −5.263 | −37.585 | 1.00 | 17.30 | C | N |
| ATOM | 10105 | C | LYS I | 433 | 37.934 | −2.543 | −34.128 | 1.00 | 11.65 | C | C |
| ATOM | 10106 | O | LYS I | 433 | 38.620 | −2.281 | −33.140 | 1.00 | 11.48 | C | O |
| ATOM | 10107 | N | THR I | 434 | 37.059 | −1.691 | −34.651 | 1.00 | 11.28 | C | N |
| ATOM | 10108 | CA | THR I | 434 | 36.764 | −0.407 | −34.027 | 1.00 | 11.21 | C | C |
| ATOM | 10109 | CB | THR I | 434 | 37.809 | 0.653 | −34.414 | 1.00 | 11.30 | C | C |
| ATOM | 10110 | OG1 | THR I | 434 | 37.324 | 1.955 | −34.062 | 1.00 | 11.83 | C | O |
| ATOM | 10111 | CG2 | THR I | 434 | 38.088 | 0.608 | −35.910 | 1.00 | 11.84 | C | C |
| ATOM | 10112 | C | THR I | 434 | 35.380 | 0.083 | −34.446 | 1.00 | 10.94 | C | C |
| ATOM | 10113 | O | THR I | 434 | 34.982 | −0.085 | −35.597 | 1.00 | 10.93 | C | O |
| ATOM | 10114 | N | PHE I | 435 | 34.604 | 0.572 | −33.484 | 1.00 | 11.10 | C | N |
| ATOM | 10115 | CA | PHE I | 435 | 33.176 | 0.281 | −33.466 | 1.00 | 11.96 | C | C |
| ATOM | 10116 | CB | PHE I | 435 | 32.726 | −0.248 | −32.106 | 1.00 | 11.51 | C | C |
| ATOM | 10117 | CG | PHE I | 435 | 32.896 | −1.732 | −31.957 | 1.00 | 11.19 | C | C |
| ATOM | 10118 | CD1 | PHE I | 435 | 33.805 | −2.246 | −31.047 | 1.00 | 10.86 | C | C |
| ATOM | 10119 | CE1 | PHE I | 435 | 34.022 | −3.608 | −30.957 | 1.00 | 10.24 | C | C |
| ATOM | 10120 | CZ | PHE I | 435 | 33.375 | −4.467 | −31.823 | 1.00 | 9.89 | C | C |
| ATOM | 10121 | CE2 | PHE I | 435 | 32.512 | −3.963 | −32.774 | 1.00 | 10.78 | C | C |
| ATOM | 10122 | CD2 | PHE I | 435 | 32.300 | −2.599 | −32.858 | 1.00 | 11.14 | C | C |
| ATOM | 10123 | C | PHE I | 435 | 32.255 | 1.381 | −33.984 | 1.00 | 13.00 | C | C |
| ATOM | 10124 | O | PHE I | 435 | 32.168 | 2.466 | −33.407 | 1.00 | 13.03 | C | O |
| ATOM | 10125 | N | SER I | 436 | 31.429 | 1.001 | −34.954 | 1.00 | 13.89 | C | N |
| ATOM | 10126 | CA | SER I | 436 | 30.556 | 1.929 | −35.657 | 1.00 | 14.67 | C | C |
| ATOM | 10127 | CB | SER I | 436 | 30.282 | 1.417 | −37.074 | 1.00 | 15.04 | C | C |
| ATOM | 10128 | OG | SER I | 436 | 30.877 | 2.256 | −38.049 | 1.00 | 16.58 | C | O |
| ATOM | 10129 | C | SER I | 436 | 29.240 | 2.105 | −34.903 | 1.00 | 14.75 | C | C |
| ATOM | 10130 | O | SER I | 436 | 28.192 | 1.626 | −35.340 | 1.00 | 14.75 | C | O |
| ATOM | 10131 | N | ASN I | 437 | 29.302 | 2.785 | −33.764 | 1.00 | 14.44 | C | N |
| ATOM | 10132 | CA | ASN I | 437 | 28.124 | 3.433 | −33.203 | 1.00 | 14.35 | C | C |
| ATOM | 10133 | CB | ASN I | 437 | 27.891 | 4.781 | −33.881 | 1.00 | 14.75 | C | C |
| ATOM | 10134 | CG | ASN I | 437 | 26.889 | 5.641 | −33.139 | 1.00 | 15.51 | C | C |
| ATOM | 10135 | OD1 | ASN I | 437 | 27.054 | 5.920 | −31.951 | 1.00 | 16.85 | C | O |
| ATOM | 10136 | ND2 | ASN I | 437 | 25.878 | 6.120 | −33.856 | 1.00 | 16.10 | C | N |
| ATOM | 10137 | C | ASN I | 437 | 26.885 | 2.558 | −33.345 | 1.00 | 13.69 | C | C |
| ATOM | 10138 | O | ASN I | 437 | 25.912 | 2.941 | −33.995 | 1.00 | 13.40 | C | O |
| ATOM | 10139 | N | GLY I | 438 | 26.957 | 1.358 | −32.782 | 1.00 | 13.39 | C | N |
| ATOM | 10140 | CA | GLY I | 438 | 25.931 | 0.345 | −32.986 | 1.00 | 12.92 | C | C |
| ATOM | 10141 | C | GLY I | 438 | 26.000 | −0.743 | −31.934 | 1.00 | 12.72 | C | C |
| ATOM | 10142 | O | GLY I | 438 | 26.154 | −0.459 | −30.747 | 1.00 | 12.83 | C | O |
| ATOM | 10143 | N | CYS I | 439 | 25.949 | −1.996 | −32.375 | 1.00 | 12.17 | C | N |
| ATOM | 10144 | CA | CYS I | 439 | 26.014 | −3.126 | −31.456 | 1.00 | 11.52 | C | C |
| ATOM | 10145 | CB | CYS I | 439 | 24.672 | −3.321 | −30.750 | 1.00 | 11.68 | C | C |
| ATOM | 10146 | SG | CYS I | 439 | 24.046 | −5.015 | −30.795 | 1.00 | 12.89 | C | S |
| ATOM | 10147 | C | CYS I | 439 | 26.437 | −4.411 | −32.159 | 1.00 | 10.95 | C | C |
| ATOM | 10148 | O | CYS I | 439 | 25.714 | −4.935 | −33.007 | 1.00 | 10.77 | C | O |
| ATOM | 10149 | N | ASP I | 440 | 27.617 | −4.909 | −31.802 | 1.00 | 10.41 | C | N |
| ATOM | 10150 | CA | ASP I | 440 | 28.240 | −6.017 | −32.518 | 1.00 | 10.06 | C | C |
| ATOM | 10151 | CB | ASP I | 440 | 29.514 | −5.548 | −33.224 | 1.00 | 10.85 | C | C |
| ATOM | 10152 | CG | ASP I | 440 | 29.336 | −5.417 | −34.724 | 1.00 | 13.11 | C | C |
| ATOM | 10153 | OD1 | ASP I | 440 | 29.376 | −6.453 | −35.422 | 1.00 | 14.97 | C | O |
| ATOM | 10154 | OD2 | ASP I | 440 | 29.192 | −4.275 | −35.209 | 1.00 | 15.27 | C | O |
| ATOM | 10155 | C | ASP I | 440 | 28.566 | −7.175 | −31.578 | 1.00 | 9.01 | C | C |
| ATOM | 10156 | O | ASP I | 440 | 28.479 | −7.040 | −30.358 | 1.00 | 9.09 | C | O |
| ATOM | 10157 | N | TYR I | 441 | 29.027 | −8.280 | −32.159 | 1.00 | 7.92 | C | N |
| ATOM | 10158 | CA | TYR I | 441 | 29.448 | −9.461 | −31.403 | 1.00 | 7.24 | C | C |
| ATOM | 10159 | CB | TYR I | 441 | 28.448 | −10.605 | −31.610 | 1.00 | 7.04 | C | C |
| ATOM | 10160 | CG | TYR I | 441 | 28.862 | −11.916 | −30.979 | 1.00 | 6.77 | C | C |
| ATOM | 10161 | CD1 | TYR I | 441 | 28.439 | −12.256 | −29.701 | 1.00 | 7.41 | C | C |
| ATOM | 10162 | CE1 | TYR I | 441 | 28.805 | −13.460 | −29.122 | 1.00 | 6.79 | C | C |
| ATOM | 10163 | CZ | TYR I | 441 | 29.529 | −14.381 | −29.855 | 1.00 | 6.49 | C | C |
| ATOM | 10164 | OH | TYR I | 441 | 29.907 | −15.573 | −29.277 | 1.00 | 5.09 | C | O |
| ATOM | 10165 | CE2 | TYR I | 441 | 29.946 | −14.076 | −31.134 | 1.00 | 7.52 | C | C |
| ATOM | 10166 | CD2 | TYR I | 441 | 29.600 | −12.853 | −31.694 | 1.00 | 7.12 | C | C |
| ATOM | 10167 | C | TYR I | 441 | 30.833 | −9.902 | −31.872 | 1.00 | 6.89 | C | C |
| ATOM | 10168 | O | TYR I | 441 | 31.150 | −9.809 | −33.058 | 1.00 | 7.12 | C | O |
| ATOM | 10169 | N | VAL I | 442 | 31.664 | −10.363 | −30.944 | 1.00 | 6.73 | C | N |
| ATOM | 10170 | CA | VAL I | 442 | 32.959 | −10.941 | −31.301 | 1.00 | 6.78 | C | C |
| ATOM | 10171 | CB | VAL I | 442 | 34.133 | −10.008 | −30.925 | 1.00 | 6.51 | C | C |
| ATOM | 10172 | CG1 | VAL I | 442 | 34.206 | −8.830 | −31.879 | 1.00 | 7.38 | C | C |
| ATOM | 10173 | CG2 | VAL I | 442 | 34.007 | −9.530 | −29.488 | 1.00 | 6.00 | C | C |
| ATOM | 10174 | C | VAL I | 442 | 33.142 | −12.285 | −30.609 | 1.00 | 7.13 | C | C |
| ATOM | 10175 | O | VAL I | 442 | 32.307 | −12.689 | −29.800 | 1.00 | 7.77 | C | O |
| ATOM | 10176 | N | SER I | 443 | 34.241 | −12.970 | −30.908 | 1.00 | 7.12 | C | N |
| ATOM | 10177 | CA | SER I | 443 | 34.476 | −14.283 | −30.321 | 1.00 | 7.64 | C | C |
| ATOM | 10178 | CB | SER I | 443 | 33.940 | −15.391 | −31.233 | 1.00 | 7.83 | C | C |
| ATOM | 10179 | OG | SER I | 443 | 34.994 | −16.091 | −31.870 | 1.00 | 7.99 | C | O |
| ATOM | 10180 | C | SER I | 443 | 35.932 | −14.541 | −29.940 | 1.00 | 8.16 | C | C |
| ATOM | 10181 | O | SER I | 443 | 36.841 | −13.847 | −30.399 | 1.00 | 8.69 | C | O |
| ATOM | 10182 | N | ASN I | 444 | 36.135 | −15.517 | −29.058 | 1.00 | 8.73 | C | N |
| ATOM | 10183 | CA | ASN I | 444 | 37.473 | −15.972 | −28.692 | 1.00 | 9.50 | C | C |
| ATOM | 10184 | CB | ASN I | 444 | 37.408 | −16.989 | −27.546 | 1.00 | 9.65 | C | C |
| ATOM | 10185 | CG | ASN I | 444 | 36.105 | −16.916 | −26.772 | 1.00 | 10.64 | C | C |
| ATOM | 10186 | OD1 | ASN I | 444 | 35.530 | −15.839 | −26.602 | 1.00 | 12.28 | C | O |
| ATOM | 10187 | ND2 | ASN I | 444 | 35.617 | −18.069 | −26.325 | 1.00 | 11.75 | C | N |
| ATOM | 10188 | C | ASN I | 444 | 38.240 | −16.558 | −29.877 | 1.00 | 10.24 | C | C |
| ATOM | 10189 | O | ASN I | 444 | 37.657 | −17.206 | −30.748 | 1.00 | 9.89 | C | O |
| ATOM | 10190 | N | LYS I | 445 | 39.565 | −16.467 | −29.809 | 1.00 | 11.63 | C | N |
| ATOM | 10191 | CA | LYS I | 445 | 40.394 | −16.179 | −30.979 | 1.00 | 13.07 | C | C |
| ATOM | 10192 | CB | LYS I | 445 | 40.843 | −17.470 | −31.664 | 1.00 | 13.70 | C | C |
| ATOM | 10193 | CG | LYS I | 445 | 41.782 | −17.255 | −32.848 | 1.00 | 15.80 | C | C |
| ATOM | 10194 | CD | LYS I | 445 | 42.771 | −16.114 | −32.613 | 1.00 | 18.31 | C | C |
| ATOM | 10195 | CE | LYS I | 445 | 42.855 | −15.717 | −31.146 | 1.00 | 19.03 | C | C |
| ATOM | 10196 | NZ | LYS I | 445 | 43.703 | −16.652 | −30.355 | 1.00 | 20.02 | C | N |
| ATOM | 10197 | C | LYS I | 445 | 39.701 | −15.268 | −31.980 | 1.00 | 12.96 | C | C |
| ATOM | 10198 | O | LYS I | 445 | 38.486 | −15.336 | −32.159 | 1.00 | 13.24 | C | O |
| ATOM | 10199 | N | GLY I | 446 | 40.491 | −14.448 | −32.664 | 1.00 | 13.08 | C | N |
| ATOM | 10200 | CA | GLY I | 446 | 40.131 | −13.057 | −32.889 | 1.00 | 12.42 | C | C |
| ATOM | 10201 | C | GLY I | 446 | 40.305 | −12.233 | −31.631 | 1.00 | 11.83 | C | C |
| ATOM | 10202 | O | GLY I | 446 | 41.402 | −11.756 | −31.342 | 1.00 | 12.44 | C | O |
| ATOM | 10203 | N | VAL I | 447 | 39.264 | −12.192 | −30.808 | 1.00 | 10.63 | C | N |
| ATOM | 10204 | CA | VAL I | 447 | 39.157 | −11.162 | −29.785 | 1.00 | 10.01 | C | C |
| ATOM | 10205 | CB | VAL I | 447 | 37.758 | −10.516 | −29.764 | 1.00 | 9.97 | C | C |
| ATOM | 10206 | CG1 | VAL I | 447 | 37.778 | −9.225 | −28.957 | 1.00 | 10.51 | C | C |
| ATOM | 10207 | CG2 | VAL I | 447 | 37.287 | −10.244 | −31.180 | 1.00 | 10.50 | C | C |
| ATOM | 10208 | C | VAL I | 447 | 39.548 | −11.648 | −28.390 | 1.00 | 9.53 | C | C |
| ATOM | 10209 | O | VAL I | 447 | 39.184 | −12.747 | −27.967 | 1.00 | 9.61 | C | O |
| ATOM | 10210 | N | ASP I | 448 | 40.211 | −10.766 | −27.652 | 1.00 | 9.16 | C | N |
| ATOM | 10211 | CA | ASP I | 448 | 40.803 | −11.093 | −26.362 | 1.00 | 8.75 | C | C |
| ATOM | 10212 | CB | ASP I | 448 | 42.325 | −11.159 | −26.502 | 1.00 | 9.22 | C | C |
| ATOM | 10213 | CG | ASP I | 448 | 42.975 | −12.024 | −25.443 | 1.00 | 11.44 | C | C |
| ATOM | 10214 | OD1 | ASP I | 448 | 42.372 | −12.204 | −24.365 | 1.00 | 13.83 | C | O |
| ATOM | 10215 | OD2 | ASP I | 448 | 44.119 | −12.472 | −25.665 | 1.00 | 14.13 | C | O |
| ATOM | 10216 | C | ASP I | 448 | 40.427 | −9.976 | −25.398 | 1.00 | 8.18 | C | C |
| ATOM | 10217 | O | ASP I | 448 | 40.203 | −10.209 | −24.211 | 1.00 | 8.06 | C | O |
| ATOM | 10218 | N | THR I | 449 | 40.201 | −8.794 | −25.964 | 1.00 | 7.63 | C | N |
| ATOM | 10219 | CA | THR I | 449 | 39.894 | −7.592 | −25.197 | 1.00 | 6.80 | C | C |
| ATOM | 10220 | CB | THR I | 449 | 41.165 | −6.750 | −24.940 | 1.00 | 6.89 | C | C |
| ATOM | 10221 | OG1 | THR I | 449 | 41.805 | −7.196 | −23.738 | 1.00 | 6.16 | C | O |
| ATOM | 10222 | CG2 | THR I | 449 | 40.815 | −5.275 | −24.804 | 1.00 | 7.55 | C | C |
| ATOM | 10223 | C | THR I | 449 | 38.898 | −6.751 | −25.987 | 1.00 | 6.53 | C | C |
| ATOM | 10224 | O | THR I | 449 | 38.949 | −6.708 | −27.216 | 1.00 | 6.59 | C | O |
| ATOM | 10225 | N | VAL I | 450 | 37.947 | −6.147 | −25.288 | 1.00 | 6.07 | C | N |
| ATOM | 10226 | CA | VAL I | 450 | 37.318 | −4.934 | −25.781 | 1.00 | 6.02 | C | C |
| ATOM | 10227 | CB | VAL I | 450 | 35.839 | −5.158 | −26.121 | 1.00 | 5.61 | C | C |
| ATOM | 10228 | CG1 | VAL I | 450 | 35.697 | −6.278 | −27.136 | 1.00 | 5.32 | C | C |
| ATOM | 10229 | CG2 | VAL I | 450 | 35.047 | −5.460 | −24.860 | 1.00 | 5.55 | C | C |
| ATOM | 10230 | C | VAL I | 450 | 37.427 | −3.837 | −24.743 | 1.00 | 6.37 | C | C |
| ATOM | 10231 | O | VAL I | 450 | 37.359 | −4.097 | −23.542 | 1.00 | 6.54 | C | O |
| ATOM | 10232 | N | SER I | 451 | 37.704 | −2.626 | −25.207 | 1.00 | 6.41 | C | N |
| ATOM | 10233 | CA | SER I | 451 | 37.571 | −1.451 | −24.366 | 1.00 | 6.70 | C | C |
| ATOM | 10234 | CB | SER I | 451 | 38.845 | −0.607 | −24.422 | 1.00 | 7.13 | C | C |
| ATOM | 10235 | OG | SER I | 451 | 39.573 | −0.864 | −25.610 | 1.00 | 9.26 | C | O |
| ATOM | 10236 | C | SER I | 451 | 36.373 | −0.626 | −24.802 | 1.00 | 6.47 | C | C |
| ATOM | 10237 | O | SER I | 451 | 36.100 | −0.493 | −25.994 | 1.00 | 6.54 | C | O |
| ATOM | 10238 | N | VAL I | 452 | 35.577 | −0.203 | −23.830 | 1.00 | 6.56 | C | N |
| ATOM | 10239 | CA | VAL I | 452 | 34.627 | 0.872 | −24.050 | 1.00 | 6.31 | C | C |
| ATOM | 10240 | CB | VAL I | 452 | 33.205 | 0.448 | −23.647 | 1.00 | 5.74 | C | C |
| ATOM | 10241 | CG1 | VAL I | 452 | 32.189 | 1.471 | −24.130 | 1.00 | 5.06 | C | C |
| ATOM | 10242 | CG2 | VAL I | 452 | 32.891 | −0.929 | −24.212 | 1.00 | 5.56 | C | C |
| ATOM | 10243 | C | VAL I | 452 | 35.056 | 2.087 | −23.242 | 1.00 | 6.60 | C | C |
| ATOM | 10244 | O | VAL I | 452 | 35.499 | 1.950 | −22.102 | 1.00 | 7.04 | C | O |
| ATOM | 10245 | N | GLY I | 453 | 35.141 | 3.234 | −23.907 | 1.00 | 6.54 | C | N |
| ATOM | 10246 | CA | GLY I | 453 | 35.790 | 4.395 | −23.314 | 1.00 | 6.43 | C | C |
| ATOM | 10247 | C | GLY I | 453 | 37.010 | 3.964 | −22.525 | 1.00 | 6.09 | C | C |
| ATOM | 10248 | O | GLY I | 453 | 37.910 | 3.328 | −23.071 | 1.00 | 5.90 | C | O |
| ATOM | 10249 | N | ASN I | 454 | 36.985 | 4.188 | −21.216 | 1.00 | 6.02 | C | N |
| ATOM | 10250 | CA | ASN I | 454 | 38.156 | 3.904 | −20.396 | 1.00 | 6.39 | C | C |
| ATOM | 10251 | CB | ASN I | 454 | 38.491 | 5.088 | −19.491 | 1.00 | 6.81 | C | C |
| ATOM | 10252 | CG | ASN I | 454 | 39.518 | 6.009 | −20.108 | 1.00 | 8.99 | C | C |
| ATOM | 10253 | OD1 | ASN I | 454 | 40.718 | 5.817 | −19.912 | 1.00 | 11.67 | C | O |
| ATOM | 10254 | ND2 | ASN I | 454 | 39.073 | 6.836 | −21.048 | 1.00 | 10.25 | C | N |
| ATOM | 10255 | C | ASN I | 454 | 38.053 | 2.617 | −19.588 | 1.00 | 6.15 | C | C |
| ATOM | 10256 | O | ASN I | 454 | 39.041 | 2.154 | −19.016 | 1.00 | 6.47 | C | O |
| ATOM | 10257 | N | THR I | 455 | 36.888 | 1.983 | −19.635 | 1.00 | 5.40 | C | N |
| ATOM | 10258 | CA | THR I | 455 | 36.730 | 0.659 | −19.057 | 1.00 | 4.53 | C | C |
| ATOM | 10259 | CB | THR I | 455 | 35.251 | 0.352 | −18.737 | 1.00 | 4.70 | C | C |
| ATOM | 10260 | OG1 | THR I | 455 | 34.662 | 1.471 | −18.063 | 1.00 | 5.28 | C | O |
| ATOM | 10261 | CG2 | THR I | 455 | 35.141 | −0.878 | −17.845 | 1.00 | 5.57 | C | C |
| ATOM | 10262 | C | THR I | 455 | 37.296 | −0.399 | −19.999 | 1.00 | 4.01 | C | C |
| ATOM | 10263 | O | THR I | 455 | 37.333 | −0.204 | −21.216 | 1.00 | 3.78 | C | O |
| ATOM | 10264 | N | LEU I | 456 | 37.885 | −1.438 | −19.414 | 1.00 | 3.87 | C | N |
| ATOM | 10265 | CA | LEU I | 456 | 38.439 | −2.542 | −20.189 | 1.00 | 4.05 | C | C |
| ATOM | 10266 | CB | LEU I | 456 | 39.946 | −2.660 | −19.955 | 1.00 | 3.95 | C | C |
| ATOM | 10267 | CG | LEU I | 456 | 40.690 | −3.636 | −20.868 | 1.00 | 3.76 | C | C |
| ATOM | 10268 | CD1 | LEU I | 456 | 40.594 | −3.192 | −22.319 | 1.00 | 5.35 | C | C |
| ATOM | 10269 | CD2 | LEU I | 456 | 42.142 | −3.770 | −20.440 | 1.00 | 4.10 | C | C |
| ATOM | 10270 | C | LEU I | 456 | 37.755 | −3.858 | −19.845 | 1.00 | 4.47 | C | C |
| ATOM | 10271 | O | LEU I | 456 | 37.769 | −4.293 | −18.694 | 1.00 | 4.94 | C | O |
| ATOM | 10272 | N | TYR I | 457 | 37.248 | −4.534 | −20.869 | 1.00 | 4.59 | C | N |
| ATOM | 10273 | CA | TYR I | 457 | 36.626 | −5.839 | −20.695 | 1.00 | 4.60 | C | C |
| ATOM | 10274 | CB | TYR I | 457 | 35.248 | −5.861 | −21.363 | 1.00 | 4.71 | C | C |
| ATOM | 10275 | CG | TYR I | 457 | 34.244 | −4.912 | −20.739 | 1.00 | 5.62 | C | C |
| ATOM | 10276 | CD1 | TYR I | 457 | 34.230 | −3.563 | −21.074 | 1.00 | 7.09 | C | C |
| ATOM | 10277 | CE1 | TYR I | 457 | 33.316 | −2.697 | −20.510 | 1.00 | 8.22 | C | C |
| ATOM | 10278 | CZ | TYR I | 457 | 32.408 | −3.173 | −19.588 | 1.00 | 8.06 | C | C |
| ATOM | 10279 | OH | TYR I | 457 | 31.469 | −2.318 | −19.059 | 1.00 | 8.80 | C | O |
| ATOM | 10280 | CE2 | TYR I | 457 | 32.381 | −4.513 | −19.262 | 1.00 | 7.18 | C | C |
| ATOM | 10281 | CD2 | TYR I | 457 | 33.302 | −5.371 | −19.827 | 1.00 | 6.66 | C | C |
| ATOM | 10282 | C | TYR I | 457 | 37.513 | −6.932 | −21.285 | 1.00 | 4.46 | C | C |
| ATOM | 10283 | O | TYR I | 457 | 38.049 | −6.780 | −22.383 | 1.00 | 4.92 | C | O |
| ATOM | 10284 | N | TYR I | 458 | 37.706 | −8.009 | −20.529 | 1.00 | 3.90 | C | N |
| ATOM | 10285 | CA | TYR I | 458 | 38.520 | −9.138 | −20.980 | 1.00 | 3.26 | C | C |
| ATOM | 10286 | CB | TYR I | 458 | 39.407 | −9.632 | −19.832 | 1.00 | 3.16 | C | C |
| ATOM | 10287 | CG | TYR I | 458 | 40.399 | −8.610 | −19.320 | 1.00 | 3.29 | C | C |
| ATOM | 10288 | CD1 | TYR I | 458 | 40.076 | −7.762 | −18.269 | 1.00 | 3.93 | C | C |
| ATOM | 10289 | CE1 | TYR I | 458 | 40.993 | −6.848 | −17.782 | 1.00 | 4.90 | C | C |
| ATOM | 10290 | CZ | TYR I | 458 | 42.245 | −6.766 | −18.354 | 1.00 | 5.12 | C | C |
| ATOM | 10291 | OH | TYR I | 458 | 43.156 | −5.850 | −17.881 | 1.00 | 5.42 | C | O |
| ATOM | 10292 | CE2 | TYR I | 458 | 42.590 | −7.599 | −19.395 | 1.00 | 4.72 | C | C |
| ATOM | 10293 | CD2 | TYR I | 458 | 41.677 | −8.528 | −19.858 | 1.00 | 3.89 | C | C |
| ATOM | 10294 | C | TYR I | 458 | 37.638 | −10.293 | −21.464 | 1.00 | 3.04 | C | C |
| ATOM | 10295 | O | TYR I | 458 | 36.897 | −10.869 | −20.672 | 1.00 | 3.37 | C | O |
| ATOM | 10296 | N | VAL I | 459 | 37.857 | −10.755 | −22.696 | 1.00 | 2.71 | C | N |
| ATOM | 10297 | CA | VAL I | 459 | 36.818 | −11.487 | −23.441 | 1.00 | 2.61 | C | C |
| ATOM | 10298 | CB | VAL I | 459 | 36.856 | −11.172 | −24.940 | 1.00 | 2.22 | C | C |
| ATOM | 10299 | CG1 | VAL I | 459 | 35.936 | −12.119 | −25.698 | 1.00 | 2.62 | C | C |
| ATOM | 10300 | CG2 | VAL I | 459 | 36.459 | −9.728 | −25.187 | 1.00 | 2.61 | C | C |
| ATOM | 10301 | C | VAL I | 459 | 36.847 | −13.006 | −23.279 | 1.00 | 3.03 | C | C |
| ATOM | 10302 | O | VAL I | 459 | 37.324 | −13.520 | −22.267 | 1.00 | 3.21 | C | O |
| ATOM | 10303 | N | ASN I | 460 | 36.301 | −13.724 | −24.263 | 1.00 | 106.93 | C | N |
| ATOM | 10304 | CA | ASN I | 460 | 36.733 | −15.100 | −24.460 | 1.00 | 95.54 | C | C |
| ATOM | 10305 | CB | ASN I | 460 | 37.469 | −15.533 | −23.198 | 1.00 | 20.00 | C | C |
| ATOM | 10306 | CG | ASN I | 460 | 38.132 | −16.862 | −23.352 | 1.00 | 20.00 | C | C |
| ATOM | 10307 | OD1 | ASN I | 460 | 38.781 | −17.118 | −24.365 | 1.00 | 20.00 | C | O |
| ATOM | 10308 | ND2 | ASN I | 460 | 37.824 | −17.783 | −22.448 | 1.00 | 20.00 | C | N |
| ATOM | 10309 | C | ASN I | 460 | 35.684 | −16.173 | −24.797 | 1.00 | 99.41 | C | C |
| ATOM | 10310 | O | ASN I | 460 | 35.555 | −17.141 | −24.047 | 1.00 | 108.91 | C | O |
| ATOM | 10311 | N | LYS I | 461 | 35.174 | −16.182 | −26.023 | 1.00 | 97.04 | C | N |
| ATOM | 10312 | CA | LYS I | 461 | 34.426 | −17.350 | −26.495 | 1.00 | 105.93 | C | C |
| ATOM | 10313 | CB | LYS I | 461 | 33.128 | −17.516 | −25.700 | 1.00 | 20.00 | C | C |
| ATOM | 10314 | CG | LYS I | 461 | 33.324 | −18.050 | −24.284 | 1.00 | 20.00 | C | C |
| ATOM | 10315 | CD | LYS I | 461 | 32.012 | −18.071 | −23.510 | 1.00 | 20.00 | C | C |
| ATOM | 10316 | CE | LYS I | 461 | 32.154 | −18.798 | −22.179 | 1.00 | 20.00 | C | C |
| ATOM | 10317 | NZ | LYS I | 461 | 30.869 | −19.408 | −21.736 | 1.00 | 20.00 | C | N |
| ATOM | 10318 | C | LYS I | 461 | 34.141 | −17.231 | −27.987 | 1.00 | 102.28 | C | C |
| ATOM | 10319 | O | LYS I | 461 | 34.601 | −16.289 | −28.627 | 1.00 | 105.02 | C | O |
| ATOM | 10320 | N | GLN I | 462 | 33.377 | −18.164 | −28.545 | 1.00 | 96.68 | C | N |
| ATOM | 10321 | CA | GLN I | 462 | 32.944 | −18.040 | −29.939 | 1.00 | 105.71 | C | C |
| ATOM | 10322 | CB | GLN I | 462 | 33.693 | −19.032 | −30.836 | 1.00 | 20.00 | C | C |
| ATOM | 10323 | CG | GLN I | 462 | 35.184 | −18.748 | −30.976 | 1.00 | 20.00 | C | C |
| ATOM | 10324 | CD | GLN I | 462 | 35.922 | −19.829 | −31.741 | 1.00 | 20.00 | C | C |
| ATOM | 10325 | OE1 | GLN I | 462 | 35.456 | −20.965 | −31.841 | 1.00 | 20.00 | C | O |
| ATOM | 10326 | NE2 | GLN I | 462 | 37.071 | −19.475 | −32.303 | 1.00 | 20.00 | C | N |
| ATOM | 10327 | C | GLN I | 462 | 31.440 | −18.235 | −30.082 | 1.00 | 101.50 | C | C |
| ATOM | 10328 | O | GLN I | 462 | 30.881 | −19.193 | −29.553 | 1.00 | 105.94 | C | O |
| ATOM | 10329 | N | GLU I | 463 | 30.787 | −17.335 | −30.809 | 1.00 | 98.92 | C | N |
| ATOM | 10330 | CA | GLU I | 463 | 29.339 | −17.413 | −30.975 | 1.00 | 108.59 | C | C |
| ATOM | 10331 | CB | GLU I | 463 | 28.684 | −16.055 | −30.712 | 1.00 | 20.00 | C | C |
| ATOM | 10332 | CG | GLU I | 463 | 28.617 | −15.668 | −29.238 | 1.00 | 20.00 | C | C |
| ATOM | 10333 | CD | GLU I | 463 | 28.032 | −14.283 | −29.019 | 1.00 | 20.00 | C | C |
| ATOM | 10334 | OE1 | GLU I | 463 | 27.535 | −13.682 | −29.995 | 1.00 | 20.00 | C | O |
| ATOM | 10335 | OE2 | GLU I | 463 | 27.975 | −13.842 | −27.851 | 1.00 | 20.00 | C | O |
| ATOM | 10336 | C | GLU I | 463 | 28.950 | −17.934 | −32.353 | 1.00 | 116.39 | C | C |
| ATOM | 10337 | O | GLU I | 463 | 29.396 | −17.411 | −33.375 | 1.00 | 118.70 | C | O |
| ATOM | 10338 | N | GLY I | 464 | 28.103 | −18.957 | −32.372 | 1.00 | 127.70 | C | N |
| ATOM | 10339 | CA | GLY I | 464 | 27.598 | −19.512 | −33.621 | 1.00 | 143.17 | C | C |
| ATOM | 10340 | C | GLY I | 464 | 26.466 | −18.687 | −34.200 | 1.00 | 149.77 | C | C |
| ATOM | 10341 | O | GLY I | 464 | 25.945 | −17.784 | −33.545 | 1.00 | 155.06 | C | O |
| ATOM | 10342 | N | LYS I | 465 | 26.088 | −18.997 | −35.435 | 1.00 | 153.73 | C | N |
| ATOM | 10343 | CA | LYS I | 465 | 25.034 | −18.261 | −36.124 | 1.00 | 153.69 | C | C |
| ATOM | 10344 | CB | LYS I | 465 | 23.750 | −18.259 | −35.293 | 1.00 | 20.00 | C | C |
| ATOM | 10345 | CG | LYS I | 465 | 23.099 | −19.626 | −35.151 | 1.00 | 20.00 | C | C |
| ATOM | 10346 | CD | LYS I | 465 | 21.848 | −19.555 | −34.292 | 1.00 | 20.00 | C | C |
| ATOM | 10347 | CE | LYS I | 465 | 21.188 | −20.919 | −34.164 | 1.00 | 20.00 | C | C |
| ATOM | 10348 | NZ | LYS I | 465 | 19.972 | −20.869 | −33.307 | 1.00 | 20.00 | C | N |
| ATOM | 10349 | C | LYS I | 465 | 25.466 | −16.831 | −36.426 | 1.00 | 155.37 | C | C |
| ATOM | 10350 | O | LYS I | 465 | 25.158 | −16.292 | −37.489 | 1.00 | 155.20 | C | O |
| ATOM | 10351 | N | GLU I | 472 | 20.477 | −14.304 | −43.583 | 1.00 | 145.47 | C | N |
| ATOM | 10352 | CA | GLU I | 472 | 19.435 | −13.563 | −44.284 | 1.00 | 151.12 | C | C |
| ATOM | 10353 | CB | GLU I | 472 | 18.367 | −14.521 | −44.821 | 1.00 | 20.00 | C | C |
| ATOM | 10354 | CG | GLU I | 472 | 18.856 | −15.453 | −45.922 | 1.00 | 20.00 | C | C |
| ATOM | 10355 | CD | GLU I | 472 | 17.778 | −16.409 | −46.402 | 1.00 | 20.00 | C | C |
| ATOM | 10356 | OE1 | GLU I | 472 | 16.723 | −16.504 | −45.739 | 1.00 | 20.00 | C | O |
| ATOM | 10357 | OE2 | GLU I | 472 | 17.996 | −17.083 | −47.430 | 1.00 | 20.00 | C | O |
| ATOM | 10358 | C | GLU I | 472 | 18.796 | −12.521 | −43.369 | 1.00 | 153.35 | C | C |
| ATOM | 10359 | O | GLU I | 472 | 18.205 | −12.866 | −42.345 | 1.00 | 159.41 | C | O |
| ATOM | 10360 | N | PRO I | 473 | 18.943 | −11.236 | −43.722 | 1.00 | 150.51 | C | N |
| ATOM | 10361 | CA | PRO I | 473 | 18.346 | −10.158 | −42.958 | 1.00 | 141.97 | C | C |
| ATOM | 10362 | CB | PRO I | 473 | 19.542 | −9.243 | −42.691 | 1.00 | 20.00 | C | C |
| ATOM | 10363 | CG | PRO I | 473 | 20.496 | −9.502 | −43.863 | 1.00 | 20.00 | C | C |
| ATOM | 10364 | CD | PRO I | 473 | 20.007 | −10.730 | −44.602 | 1.00 | 20.00 | C | C |
| ATOM | 10365 | C | PRO I | 473 | 17.331 | −9.429 | −43.824 | 1.00 | 135.14 | C | C |
| ATOM | 10366 | O | PRO I | 473 | 17.408 | −9.503 | −45.051 | 1.00 | 144.93 | C | O |
| ATOM | 10367 | N | ILE I | 474 | 16.371 | −8.757 | −43.197 | 1.00 | 118.07 | C | N |
| ATOM | 10368 | CA | ILE I | 474 | 15.320 | −8.071 | −43.939 | 1.00 | 105.14 | C | C |
| ATOM | 10369 | CB | ILE I | 474 | 14.440 | −9.067 | −44.737 | 1.00 | 20.00 | C | C |
| ATOM | 10370 | CG1 | ILE I | 474 | 15.255 | −9.730 | −45.856 | 1.00 | 20.00 | C | C |
| ATOM | 10371 | CD1 | ILE I | 474 | 14.525 | −10.842 | −46.588 | 1.00 | 20.00 | C | C |
| ATOM | 10372 | CG2 | ILE I | 474 | 13.224 | −8.354 | −45.317 | 1.00 | 20.00 | C | C |
| ATOM | 10373 | C | ILE I | 474 | 14.436 | −7.226 | −43.022 | 1.00 | 104.75 | C | C |
| ATOM | 10374 | O | ILE I | 474 | 13.471 | −7.725 | −42.443 | 1.00 | 111.83 | C | O |
| ATOM | 10375 | N | ILE I | 475 | 14.746 | −5.934 | −42.932 | 1.00 | 94.65 | C | N |
| ATOM | 10376 | CA | ILE I | 475 | 14.105 | −5.051 | −41.957 | 1.00 | 79.88 | C | C |
| ATOM | 10377 | CB | ILE I | 475 | 15.105 | −4.568 | −40.885 | 1.00 | 20.00 | C | C |
| ATOM | 10378 | CG1 | ILE I | 475 | 15.576 | −5.737 | −40.014 | 1.00 | 20.00 | C | C |
| ATOM | 10379 | CD1 | ILE I | 475 | 16.791 | −5.417 | −39.163 | 1.00 | 20.00 | C | C |
| ATOM | 10380 | CG2 | ILE I | 475 | 14.493 | −3.458 | −40.043 | 1.00 | 20.00 | C | C |
| ATOM | 10381 | C | ILE I | 475 | 13.459 | −3.835 | −42.617 | 1.00 | 79.52 | C | C |
| ATOM | 10382 | O | ILE I | 475 | 14.150 | −2.913 | −43.054 | 1.00 | 82.70 | C | O |
| ATOM | 10383 | N | ASN I | 476 | 12.133 | −3.791 | −42.590 | 1.00 | 69.64 | C | N |
| ATOM | 10384 | CA | ASN I | 476 | 11.404 | −2.621 | −43.048 | 1.00 | 68.88 | C | C |
| ATOM | 10385 | CB | ASN I | 476 | 9.979 | −3.002 | −43.442 | 1.00 | 20.00 | C | C |
| ATOM | 10386 | CG | ASN I | 476 | 9.939 | −3.881 | −44.672 | 1.00 | 20.00 | C | C |
| ATOM | 10387 | OD1 | ASN I | 476 | 10.854 | −3.856 | −45.496 | 1.00 | 20.00 | C | O |
| ATOM | 10388 | ND2 | ASN I | 476 | 8.918 | −4.722 | −44.762 | 1.00 | 20.00 | C | N |
| ATOM | 10389 | C | ASN I | 476 | 11.401 | −1.512 | −42.004 | 1.00 | 67.13 | C | C |
| ATOM | 10390 | O | ASN I | 476 | 11.491 | −1.781 | −40.806 | 1.00 | 73.10 | C | O |
| ATOM | 10391 | N | PHE I | 477 | 11.492 | −0.273 | −42.474 | 1.00 | 64.95 | C | N |
| ATOM | 10392 | CA | PHE I | 477 | 11.535 | 0.883 | −41.585 | 1.00 | 68.13 | C | C |
| ATOM | 10393 | CB | PHE I | 477 | 12.064 | 2.118 | −42.327 | 1.00 | 20.00 | C | C |
| ATOM | 10394 | CG | PHE I | 477 | 13.494 | 1.995 | −42.778 | 1.00 | 20.00 | C | C |
| ATOM | 10395 | CD1 | PHE I | 477 | 14.532 | 2.409 | −41.955 | 1.00 | 20.00 | C | C |
| ATOM | 10396 | CE1 | PHE I | 477 | 15.842 | 2.359 | −42.391 | 1.00 | 20.00 | C | C |
| ATOM | 10397 | CZ | PHE I | 477 | 16.127 | 1.910 | −43.665 | 1.00 | 20.00 | C | C |
| ATOM | 10398 | CE2 | PHE I | 477 | 15.100 | 1.529 | −44.506 | 1.00 | 20.00 | C | C |
| ATOM | 10399 | CD2 | PHE I | 477 | 13.791 | 1.596 | −44.070 | 1.00 | 20.00 | C | C |
| ATOM | 10400 | C | PHE I | 477 | 10.167 | 1.186 | −40.968 | 1.00 | 65.57 | C | C |
| ATOM | 10401 | O | PHE I | 477 | 10.033 | 2.109 | −40.165 | 1.00 | 49.83 | C | O |
| ATOM | 10402 | N | TYR I | 478 | 9.136 | 0.485 | −41.432 | 1.00 | 51.97 | C | N |
| ATOM | 10403 | CA | TYR I | 478 | 7.811 | 0.580 | −40.824 | 1.00 | 52.01 | C | C |
| ATOM | 10404 | CB | TYR I | 478 | 6.729 | 0.140 | −41.815 | 1.00 | 20.00 | C | C |
| ATOM | 10405 | CG | TYR I | 478 | 6.638 | 1.012 | −43.045 | 1.00 | 20.00 | C | C |
| ATOM | 10406 | CD1 | TYR I | 478 | 5.883 | 2.179 | −43.040 | 1.00 | 20.00 | C | C |
| ATOM | 10407 | CE1 | TYR I | 478 | 5.824 | 2.997 | −44.155 | 1.00 | 20.00 | C | C |
| ATOM | 10408 | CZ | TYR I | 478 | 6.512 | 2.642 | −45.300 | 1.00 | 20.00 | C | C |
| ATOM | 10409 | OH | TYR I | 478 | 6.454 | 3.447 | −46.414 | 1.00 | 20.00 | C | O |
| ATOM | 10410 | CE2 | TYR I | 478 | 7.260 | 1.484 | −45.333 | 1.00 | 20.00 | C | C |
| ATOM | 10411 | CD2 | TYR I | 478 | 7.319 | 0.677 | −44.210 | 1.00 | 20.00 | C | C |
| ATOM | 10412 | C | TYR I | 478 | 7.745 | −0.269 | −39.556 | 1.00 | 66.63 | C | C |
| ATOM | 10413 | O | TYR I | 478 | 6.674 | −0.470 | −38.983 | 1.00 | 63.45 | C | O |
| ATOM | 10414 | N | ASP I | 479 | 8.898 | −0.790 | −39.146 | 1.00 | 65.34 | C | N |
| ATOM | 10415 | CA | ASP I | 479 | 9.013 | −1.509 | −37.886 | 1.00 | 65.32 | C | C |
| ATOM | 10416 | CB | ASP I | 479 | 9.879 | −2.758 | −38.061 | 1.00 | 20.00 | C | C |
| ATOM | 10417 | CG | ASP I | 479 | 9.210 | −3.815 | −38.916 | 1.00 | 20.00 | C | C |
| ATOM | 10418 | OD1 | ASP I | 479 | 7.967 | −3.791 | −39.028 | 1.00 | 20.00 | C | O |
| ATOM | 10419 | OD2 | ASP I | 479 | 9.920 | −4.701 | −39.436 | 1.00 | 20.00 | C | O |
| ATOM | 10420 | C | ASP I | 479 | 9.590 | −0.614 | −36.796 | 1.00 | 64.83 | C | C |
| ATOM | 10421 | O | ASP I | 479 | 9.572 | −0.976 | −35.620 | 1.00 | 60.42 | C | O |
| ATOM | 10422 | N | PRO I | 480 | 10.076 | 0.579 | −37.181 | 1.00 | 47.60 | C | N |
| ATOM | 10423 | CA | PRO I | 480 | 10.722 | 1.488 | −36.253 | 1.00 | 46.52 | C | C |
| ATOM | 10424 | CB | PRO I | 480 | 11.789 | 2.143 | −37.124 | 1.00 | 20.00 | C | C |
| ATOM | 10425 | CG | PRO I | 480 | 11.140 | 2.219 | −38.487 | 1.00 | 20.00 | C | C |
| ATOM | 10426 | CD | PRO I | 480 | 10.043 | 1.155 | −38.536 | 1.00 | 20.00 | C | C |
| ATOM | 10427 | C | PRO I | 480 | 9.736 | 2.550 | −35.806 | 1.00 | 52.36 | C | C |
| ATOM | 10428 | O | PRO I | 480 | 9.629 | 3.601 | −36.438 | 1.00 | 64.15 | C | O |
| ATOM | 10429 | N | LEU I | 481 | 8.945 | 2.229 | −34.792 | 1.00 | 62.90 | C | N |
| ATOM | 10430 | CA | LEU I | 481 | 8.005 | 3.187 | −34.233 | 1.00 | 54.24 | C | C |
| ATOM | 10431 | CB | LEU I | 481 | 6.576 | 2.656 | −34.340 | 1.00 | 20.00 | C | C |
| ATOM | 10432 | CG | LEU I | 481 | 6.056 | 2.404 | −35.756 | 1.00 | 20.00 | C | C |
| ATOM | 10433 | CD1 | LEU I | 481 | 4.735 | 1.662 | −35.704 | 1.00 | 20.00 | C | C |
| ATOM | 10434 | CD2 | LEU I | 481 | 5.911 | 3.711 | −36.524 | 1.00 | 20.00 | C | C |
| ATOM | 10435 | C | LEU I | 481 | 8.346 | 3.496 | −32.783 | 1.00 | 59.81 | C | C |
| ATOM | 10436 | O | LEU I | 481 | 9.346 | 3.015 | −32.250 | 1.00 | 64.98 | C | O |
| ATOM | 10437 | N | VAL I | 482 | 7.562 | 4.375 | −32.176 | 1.00 | 59.66 | C | N |
| ATOM | 10438 | CA | VAL I | 482 | 7.706 | 4.645 | −30.761 | 1.00 | 49.67 | C | C |
| ATOM | 10439 | CB | VAL I | 482 | 7.767 | 6.146 | −30.484 | 1.00 | 20.00 | C | C |
| ATOM | 10440 | CG1 | VAL I | 482 | 8.060 | 6.388 | −29.015 | 1.00 | 20.00 | C | C |
| ATOM | 10441 | CG2 | VAL I | 482 | 8.826 | 6.795 | −31.358 | 1.00 | 20.00 | C | C |
| ATOM | 10442 | C | VAL I | 482 | 6.550 | 4.037 | −29.987 | 1.00 | 53.03 | C | C |
| ATOM | 10443 | O | VAL I | 482 | 5.423 | 3.998 | −30.476 | 1.00 | 46.27 | C | O |
| ATOM | 10444 | N | PHE I | 483 | 6.864 | 3.441 | −28.842 | 1.00 | 61.61 | C | N |
| ATOM | 10445 | CA | PHE I | 483 | 5.847 | 2.831 | −27.994 | 1.00 | 65.03 | C | C |
| ATOM | 10446 | CB | PHE I | 483 | 6.218 | 1.380 | −27.662 | 1.00 | 20.00 | C | C |
| ATOM | 10447 | CG | PHE I | 483 | 6.212 | 0.463 | −28.857 | 1.00 | 20.00 | C | C |
| ATOM | 10448 | CD1 | PHE I | 483 | 5.036 | −0.124 | −29.292 | 1.00 | 20.00 | C | C |
| ATOM | 10449 | CE1 | PHE I | 483 | 5.028 | −0.963 | −30.390 | 1.00 | 20.00 | C | C |
| ATOM | 10450 | CZ | PHE I | 483 | 6.201 | −1.222 | −31.068 | 1.00 | 20.00 | C | C |
| ATOM | 10451 | CE2 | PHE I | 483 | 7.379 | −0.653 | −30.639 | 1.00 | 20.00 | C | C |
| ATOM | 10452 | CD2 | PHE I | 483 | 7.382 | 0.185 | −29.540 | 1.00 | 20.00 | C | C |
| ATOM | 10453 | C | PHE I | 483 | 5.627 | 3.653 | −26.723 | 1.00 | 71.00 | C | C |
| ATOM | 10454 | O | PHE I | 483 | 5.934 | 3.199 | −25.620 | 1.00 | 100.44 | C | O |
| ATOM | 10455 | N | PRO I | 484 | 5.255 | 4.928 | −26.903 | 1.00 | 54.44 | C | N |
| ATOM | 10456 | CA | PRO I | 484 | 5.000 | 5.815 | −25.781 | 1.00 | 47.46 | C | C |
| ATOM | 10457 | CB | PRO I | 484 | 6.136 | 6.828 | −25.903 | 1.00 | 20.00 | C | C |
| ATOM | 10458 | CG | PRO I | 484 | 6.468 | 6.851 | −27.406 | 1.00 | 20.00 | C | C |
| ATOM | 10459 | CD | PRO I | 484 | 5.760 | 5.686 | −28.058 | 1.00 | 20.00 | C | C |
| ATOM | 10460 | C | PRO I | 484 | 3.661 | 6.533 | −25.944 | 1.00 | 41.56 | C | C |
| ATOM | 10461 | O | PRO I | 484 | 3.430 | 7.190 | −26.959 | 1.00 | 46.53 | C | O |
| ATOM | 10462 | N | SER I | 485 | 2.779 | 6.380 | −24.964 | 1.00 | 46.30 | C | N |
| ATOM | 10463 | CA | SER I | 485 | 1.436 | 6.938 | −25.048 | 1.00 | 49.30 | C | C |
| ATOM | 10464 | CB | SER I | 485 | 0.892 | 7.220 | −23.649 | 1.00 | 20.00 | C | C |
| ATOM | 10465 | OG | SER I | 485 | 1.684 | 8.188 | −22.983 | 1.00 | 20.00 | C | O |
| ATOM | 10466 | C | SER I | 485 | 1.380 | 8.204 | −25.901 | 1.00 | 41.24 | C | C |
| ATOM | 10467 | O | SER I | 485 | 0.643 | 8.262 | −26.883 | 1.00 | 46.93 | C | O |
| ATOM | 10468 | N | ASP I | 486 | 2.103 | 9.238 | −25.482 | 1.00 | 49.48 | C | N |
| ATOM | 10469 | CA | ASP I | 486 | 2.085 | 10.518 | −26.185 | 1.00 | 37.44 | C | C |
| ATOM | 10470 | CB | ASP I | 486 | 3.029 | 11.514 | −25.511 | 1.00 | 20.00 | C | C |
| ATOM | 10471 | CG | ASP I | 486 | 2.509 | 11.993 | −24.173 | 1.00 | 20.00 | C | C |
| ATOM | 10472 | OD1 | ASP I | 486 | 1.314 | 11.773 | −23.887 | 1.00 | 20.00 | C | O |
| ATOM | 10473 | OD2 | ASP I | 486 | 3.299 | 12.566 | −23.395 | 1.00 | 20.00 | C | O |
| ATOM | 10474 | C | ASP I | 486 | 2.456 | 10.359 | −27.656 | 1.00 | 45.66 | C | C |
| ATOM | 10475 | O | ASP I | 486 | 1.752 | 10.848 | −28.539 | 1.00 | 39.86 | C | O |
| ATOM | 10476 | N | GLU I | 487 | 3.559 | 9.665 | −27.914 | 1.00 | 49.20 | C | N |
| ATOM | 10477 | CA | GLU I | 487 | 4.006 | 9.422 | −29.280 | 1.00 | 45.70 | C | C |
| ATOM | 10478 | CB | GLU I | 487 | 5.346 | 8.684 | −29.288 | 1.00 | 20.00 | C | C |
| ATOM | 10479 | CG | GLU I | 487 | 6.491 | 9.481 | −28.685 | 1.00 | 20.00 | C | C |
| ATOM | 10480 | CD | GLU I | 487 | 7.766 | 8.669 | −28.551 | 1.00 | 20.00 | C | C |
| ATOM | 10481 | OE1 | GLU I | 487 | 7.722 | 7.447 | −28.804 | 1.00 | 20.00 | C | O |
| ATOM | 10482 | OE2 | GLU I | 487 | 8.794 | 9.238 | −28.122 | 1.00 | 20.00 | C | O |
| ATOM | 10483 | C | GLU I | 487 | 2.966 | 8.648 | −30.085 | 1.00 | 41.48 | C | C |
| ATOM | 10484 | O | GLU I | 487 | 2.656 | 9.011 | −31.219 | 1.00 | 48.92 | C | O |
| ATOM | 10485 | N | PHE I | 488 | 2.402 | 7.606 | −29.483 | 1.00 | 42.16 | C | N |
| ATOM | 10486 | CA | PHE I | 488 | 1.397 | 6.789 | −30.155 | 1.00 | 39.33 | C | C |
| ATOM | 10487 | CB | PHE I | 488 | 0.968 | 5.623 | −29.268 | 1.00 | 20.00 | C | C |
| ATOM | 10488 | CG | PHE I | 488 | 2.059 | 4.633 | −29.005 | 1.00 | 20.00 | C | C |
| ATOM | 10489 | CD1 | PHE I | 488 | 2.403 | 3.694 | −29.958 | 1.00 | 20.00 | C | C |
| ATOM | 10490 | CE1 | PHE I | 488 | 3.409 | 2.779 | −29.715 | 1.00 | 20.00 | C | C |
| ATOM | 10491 | CZ | PHE I | 488 | 4.089 | 2.807 | −28.517 | 1.00 | 20.00 | C | C |
| ATOM | 10492 | CE2 | PHE I | 488 | 3.752 | 3.737 | −27.560 | 1.00 | 20.00 | C | C |
| ATOM | 10493 | CD2 | PHE I | 488 | 2.752 | 4.652 | −27.810 | 1.00 | 20.00 | C | C |
| ATOM | 10494 | C | PHE I | 488 | 0.182 | 7.618 | −30.540 | 1.00 | 43.06 | C | C |
| ATOM | 10495 | O | PHE I | 488 | −0.328 | 7.508 | −31.654 | 1.00 | 61.71 | C | O |
| ATOM | 10496 | N | ASP I | 489 | −0.261 | 8.471 | −29.624 | 1.00 | 41.95 | C | N |
| ATOM | 10497 | CA | ASP I | 489 | −1.454 | 9.273 | −29.851 | 1.00 | 42.75 | C | C |
| ATOM | 10498 | CB | ASP I | 489 | −1.837 | 10.023 | −28.581 | 1.00 | 20.00 | C | C |
| ATOM | 10499 | CG | ASP I | 489 | −2.352 | 9.102 | −27.504 | 1.00 | 20.00 | C | C |
| ATOM | 10500 | OD1 | ASP I | 489 | −2.808 | 7.989 | −27.848 | 1.00 | 20.00 | C | O |
| ATOM | 10501 | OD2 | ASP I | 489 | −2.184 | 9.435 | −26.313 | 1.00 | 20.00 | C | O |
| ATOM | 10502 | C | ASP I | 489 | −1.255 | 10.256 | −30.994 | 1.00 | 45.61 | C | C |
| ATOM | 10503 | O | ASP I | 489 | −2.206 | 10.617 | −31.689 | 1.00 | 54.39 | C | O |
| ATOM | 10504 | N | ALA I | 490 | −0.020 | 10.714 | −31.160 | 1.00 | 42.89 | C | N |
| ATOM | 10505 | CA | ALA I | 490 | 0.306 | 11.663 | −32.215 | 1.00 | 38.40 | C | C |
| ATOM | 10506 | CB | ALA I | 490 | 1.684 | 12.262 | −31.976 | 1.00 | 20.00 | C | C |
| ATOM | 10507 | C | ALA I | 490 | 0.240 | 10.994 | −33.585 | 1.00 | 39.67 | C | C |
| ATOM | 10508 | O | ALA I | 490 | −0.356 | 11.529 | −34.521 | 1.00 | 56.80 | C | O |
| ATOM | 10509 | N | SER I | 491 | 0.803 | 9.793 | −33.676 | 1.00 | 40.38 | C | N |
| ATOM | 10510 | CA | SER I | 491 | 0.721 | 8.992 | −34.893 | 1.00 | 41.77 | C | C |
| ATOM | 10511 | CB | SER I | 491 | 1.611 | 7.752 | −34.784 | 1.00 | 20.00 | C | C |
| ATOM | 10512 | OG | SER I | 491 | 1.132 | 6.864 | −33.788 | 1.00 | 20.00 | C | O |
| ATOM | 10513 | C | SER I | 491 | −0.717 | 8.580 | −35.187 | 1.00 | 39.01 | C | C |
| ATOM | 10514 | O | SER I | 491 | −1.142 | 8.563 | −36.342 | 1.00 | 36.91 | C | O |
| ATOM | 10515 | N | ILE I | 492 | −1.474 | 8.286 | −34.135 | 1.00 | 38.11 | C | N |
| ATOM | 10516 | CA | ILE I | 492 | −2.896 | 8.000 | −34.280 | 1.00 | 41.21 | C | C |
| ATOM | 10517 | CB | ILE I | 492 | −3.539 | 7.629 | −32.938 | 1.00 | 20.00 | C | C |
| ATOM | 10518 | CG1 | ILE I | 492 | −2.964 | 6.311 | −32.424 | 1.00 | 20.00 | C | C |
| ATOM | 10519 | CD1 | ILE I | 492 | −3.501 | 5.906 | −31.076 | 1.00 | 20.00 | C | C |
| ATOM | 10520 | CG2 | ILE I | 492 | −5.050 | 7.533 | −33.080 | 1.00 | 20.00 | C | C |
| ATOM | 10521 | C | ILE I | 492 | −3.646 | 9.182 | −34.883 | 1.00 | 39.22 | C | C |
| ATOM | 10522 | O | ILE I | 492 | −4.494 | 9.008 | −35.758 | 1.00 | 47.54 | C | O |
| ATOM | 10523 | N | SER I | 493 | −3.334 | 10.383 | −34.408 | 1.00 | 41.64 | C | N |
| ATOM | 10524 | CA | SER I | 493 | −3.929 | 11.598 | −34.952 | 1.00 | 35.15 | C | C |
| ATOM | 10525 | CB | SER I | 493 | −3.483 | 12.820 | −34.146 | 1.00 | 20.00 | C | C |
| ATOM | 10526 | OG | SER I | 493 | −2.100 | 13.070 | −34.322 | 1.00 | 20.00 | C | O |
| ATOM | 10527 | C | SER I | 493 | −3.547 | 11.773 | −36.416 | 1.00 | 47.90 | C | C |
| ATOM | 10528 | O | SER I | 493 | −4.380 | 12.128 | −37.250 | 1.00 | 50.94 | C | O |
| ATOM | 10529 | N | GLN I | 494 | −2.296 | 11.457 | −36.727 | 1.00 | 42.51 | C | N |
| ATOM | 10530 | CA | GLN I | 494 | −1.784 | 11.587 | −38.081 | 1.00 | 41.96 | C | C |
| ATOM | 10531 | CB | GLN I | 494 | −0.263 | 11.405 | −38.085 | 1.00 | 20.00 | C | C |
| ATOM | 10532 | CG | GLN I | 494 | 0.495 | 12.482 | −37.308 | 1.00 | 20.00 | C | C |
| ATOM | 10533 | CD | GLN I | 494 | 1.989 | 12.210 | −37.223 | 1.00 | 20.00 | C | C |
| ATOM | 10534 | OE1 | GLN I | 494 | 2.410 | 11.104 | −36.881 | 1.00 | 20.00 | C | O |
| ATOM | 10535 | NE2 | GLN I | 494 | 2.790 | 13.248 | −37.433 | 1.00 | 20.00 | C | N |
| ATOM | 10536 | C | GLN I | 494 | −2.449 | 10.583 | −39.023 | 1.00 | 33.86 | C | C |
| ATOM | 10537 | O | GLN I | 494 | −2.944 | 10.950 | −40.090 | 1.00 | 54.89 | C | O |
| ATOM | 10538 | N | VAL I | 495 | −2.511 | 9.328 | −38.594 | 1.00 | 34.99 | C | N |
| ATOM | 10539 | CA | VAL I | 495 | −3.226 | 8.300 | −39.341 | 1.00 | 38.42 | C | C |
| ATOM | 10540 | CB | VAL I | 495 | −3.245 | 6.968 | −38.576 | 1.00 | 20.00 | C | C |
| ATOM | 10541 | CG1 | VAL I | 495 | −4.032 | 5.922 | −39.349 | 1.00 | 20.00 | C | C |
| ATOM | 10542 | CG2 | VAL I | 495 | −1.828 | 6.497 | −38.309 | 1.00 | 20.00 | C | C |
| ATOM | 10543 | C | VAL I | 495 | −4.661 | 8.731 | −39.618 | 1.00 | 42.74 | C | C |
| ATOM | 10544 | O | VAL I | 495 | −5.144 | 8.634 | −40.747 | 1.00 | 44.00 | C | O |
| ATOM | 10545 | N | ASN I | 496 | −5.314 | 9.268 | −38.592 | 1.00 | 42.45 | C | N |
| ATOM | 10546 | CA | ASN I | 496 | −6.705 | 9.685 | −38.697 | 1.00 | 43.01 | C | C |
| ATOM | 10547 | CB | ASN I | 496 | −7.232 | 10.134 | −37.333 | 1.00 | 20.00 | C | C |
| ATOM | 10548 | CG | ASN I | 496 | −7.416 | 8.977 | −36.374 | 1.00 | 20.00 | C | C |
| ATOM | 10549 | OD1 | ASN I | 496 | −7.439 | 7.817 | −36.784 | 1.00 | 20.00 | C | O |
| ATOM | 10550 | ND2 | ASN I | 496 | −7.495 | 9.283 | −35.083 | 1.00 | 20.00 | C | N |
| ATOM | 10551 | C | ASN I | 496 | −6.906 | 10.790 | −39.727 | 1.00 | 43.13 | C | C |
| ATOM | 10552 | O | ASN I | 496 | −7.893 | 10.793 | −40.462 | 1.00 | 54.76 | C | O |
| ATOM | 10553 | N | GLU I | 497 | −5.990 | 11.752 | −39.744 | 1.00 | 42.08 | C | N |
| ATOM | 10554 | CA | GLU I | 497 | −6.038 | 12.828 | −40.726 | 1.00 | 43.13 | C | C |
| ATOM | 10555 | CB | GLU I | 497 | −4.947 | 13.863 | −40.443 | 1.00 | 20.00 | C | C |
| ATOM | 10556 | CG | GLU I | 497 | −5.172 | 14.672 | −39.175 | 1.00 | 20.00 | C | C |
| ATOM | 10557 | CD | GLU I | 497 | −4.045 | 15.647 | −38.896 | 1.00 | 20.00 | C | C |
| ATOM | 10558 | OE1 | GLU I | 497 | −3.007 | 15.574 | −39.587 | 1.00 | 20.00 | C | O |
| ATOM | 10559 | OE2 | GLU I | 497 | −4.192 | 16.480 | −37.977 | 1.00 | 20.00 | C | O |
| ATOM | 10560 | C | GLU I | 497 | −5.884 | 12.275 | −42.137 | 1.00 | 49.77 | C | C |
| ATOM | 10561 | O | GLU I | 497 | −6.542 | 12.736 | −43.071 | 1.00 | 42.88 | C | O |
| ATOM | 10562 | N | LYS I | 498 | −5.051 | 11.249 | −42.273 | 1.00 | 41.82 | C | N |
| ATOM | 10563 | CA | LYS I | 498 | −4.855 | 10.579 | −43.552 | 1.00 | 53.60 | C | C |
| ATOM | 10564 | CB | LYS I | 498 | −3.676 | 9.609 | −43.471 | 1.00 | 20.00 | C | C |
| ATOM | 10565 | CG | LYS I | 498 | −2.336 | 10.274 | −43.228 | 1.00 | 20.00 | C | C |
| ATOM | 10566 | CD | LYS I | 498 | −1.193 | 9.356 | −43.638 | 1.00 | 20.00 | C | C |
| ATOM | 10567 | CE | LYS I | 498 | 0.160 | 9.937 | −43.247 | 1.00 | 20.00 | C | C |
| ATOM | 10568 | NZ | LYS I | 498 | 1.285 | 9.250 | −43.942 | 1.00 | 20.00 | C | N |
| ATOM | 10569 | C | LYS I | 498 | −6.109 | 9.830 | −43.994 | 1.00 | 55.29 | C | C |
| ATOM | 10570 | O | LYS I | 498 | −6.409 | 9.763 | −45.186 | 1.00 | 61.06 | C | O |
| ATOM | 10571 | N | ILE I | 499 | −6.766 | 9.165 | −43.047 | 1.00 | 52.22 | C | N |
| ATOM | 10572 | CA | ILE I | 499 | −8.038 | 8.500 | −43.323 | 1.00 | 48.26 | C | C |
| ATOM | 10573 | CB | ILE I | 499 | −8.586 | 7.785 | −42.076 | 1.00 | 20.00 | C | C |
| ATOM | 10574 | CG1 | ILE I | 499 | −7.657 | 6.642 | −41.667 | 1.00 | 20.00 | C | C |
| ATOM | 10575 | CD1 | ILE I | 499 | −7.934 | 6.102 | −40.286 | 1.00 | 20.00 | C | C |
| ATOM | 10576 | CG2 | ILE I | 499 | −9.994 | 7.267 | −42.333 | 1.00 | 20.00 | C | C |
| ATOM | 10577 | C | ILE I | 499 | −9.067 | 9.516 | −43.798 | 1.00 | 49.07 | C | C |
| ATOM | 10578 | O | ILE I | 499 | −9.858 | 9.244 | −44.701 | 1.00 | 58.45 | C | O |
| ATOM | 10579 | N | ASN I | 500 | −8.961 | 10.726 | −43.265 | 1.00 | 51.02 | C | N |
| ATOM | 10580 | CA | ASN I | 500 | −9.845 | 11.812 | −43.644 | 1.00 | 52.06 | C | C |
| ATOM | 10581 | CB | ASN I | 500 | −9.741 | 12.940 | −42.625 | 1.00 | 20.00 | C | C |
| ATOM | 10582 | CG | ASN I | 500 | −10.861 | 13.933 | −42.753 | 1.00 | 20.00 | C | C |
| ATOM | 10583 | OD1 | ASN I | 500 | −11.927 | 13.622 | −43.286 | 1.00 | 20.00 | C | O |
| ATOM | 10584 | ND2 | ASN I | 500 | −10.647 | 15.126 | −42.232 | 1.00 | 20.00 | C | N |
| ATOM | 10585 | C | ASN I | 500 | −9.552 | 12.345 | −45.041 | 1.00 | 48.31 | C | C |
| ATOM | 10586 | O | ASN I | 500 | −10.448 | 12.834 | −45.729 | 1.00 | 56.89 | C | O |
| ATOM | 10587 | N | GLN I | 501 | −8.283 | 12.300 | −45.434 | 1.00 | 55.87 | C | N |
| ATOM | 10588 | CA | GLN I | 501 | −7.900 | 12.610 | −46.807 | 1.00 | 53.55 | C | C |
| ATOM | 10589 | CB | GLN I | 501 | −6.384 | 12.797 | −46.917 | 1.00 | 20.00 | C | C |
| ATOM | 10590 | CG | GLN I | 501 | −5.851 | 13.987 | −46.136 | 1.00 | 20.00 | C | C |
| ATOM | 10591 | CD | GLN I | 501 | −4.338 | 14.093 | −46.191 | 1.00 | 20.00 | C | C |
| ATOM | 10592 | OE1 | GLN I | 501 | −3.648 | 13.146 | −46.576 | 1.00 | 20.00 | C | O |
| ATOM | 10593 | NE2 | GLN I | 501 | −3.814 | 15.247 | −45.793 | 1.00 | 20.00 | C | N |
| ATOM | 10594 | C | GLN I | 501 | −8.383 | 11.531 | −47.777 | 1.00 | 45.67 | C | C |
| ATOM | 10595 | O | GLN I | 501 | −8.980 | 11.837 | −48.810 | 1.00 | 58.31 | C | O |
| ATOM | 10596 | N | SER I | 502 | −8.234 | 10.273 | −47.378 | 1.00 | 43.44 | C | N |
| ATOM | 10597 | CA | SER I | 502 | −8.704 | 9.157 | −48.189 | 1.00 | 45.40 | C | C |
| ATOM | 10598 | CB | SER I | 502 | −8.367 | 7.830 | −47.512 | 1.00 | 20.00 | C | C |
| ATOM | 10599 | OG | SER I | 502 | −9.001 | 7.735 | −46.250 | 1.00 | 20.00 | C | O |
| ATOM | 10600 | C | SER I | 502 | −10.206 | 9.254 | −48.413 | 1.00 | 55.15 | C | C |
| ATOM | 10601 | O | SER I | 502 | −10.708 | 8.934 | −49.490 | 1.00 | 52.57 | C | O |
| ATOM | 10602 | N | LEU I | 503 | −10.920 | 9.673 | −47.376 | 1.00 | 55.35 | C | N |
| ATOM | 10603 | CA | LEU I | 503 | −12.366 | 9.798 | −47.447 | 1.00 | 52.12 | C | C |
| ATOM | 10604 | CB | LEU I | 503 | −12.950 | 10.025 | −46.053 | 1.00 | 20.00 | C | C |
| ATOM | 10605 | CG | LEU I | 503 | −12.783 | 8.856 | −45.082 | 1.00 | 20.00 | C | C |
| ATOM | 10606 | CD1 | LEU I | 503 | −13.061 | 9.290 | −43.653 | 1.00 | 20.00 | C | C |
| ATOM | 10607 | CD2 | LEU I | 503 | −13.685 | 7.704 | −45.481 | 1.00 | 20.00 | C | C |
| ATOM | 10608 | C | LEU I | 503 | −12.761 | 10.938 | −48.371 | 1.00 | 55.74 | C | C |
| ATOM | 10609 | O | LEU I | 503 | −13.782 | 10.869 | −49.052 | 1.00 | 57.75 | C | O |
| ATOM | 10610 | N | ALA I | 504 | −11.951 | 11.991 | −48.382 | 1.00 | 57.22 | C | N |
| ATOM | 10611 | CA | ALA I | 504 | −12.187 | 13.129 | −49.261 | 1.00 | 59.12 | C | C |
| ATOM | 10612 | CB | ALA I | 504 | −11.333 | 14.315 | −48.831 | 1.00 | 20.00 | C | C |
| ATOM | 10613 | C | ALA I | 504 | −11.902 | 12.760 | −50.713 | 1.00 | 58.10 | C | C |
| ATOM | 10614 | O | ALA I | 504 | −12.656 | 13.119 | −51.618 | 1.00 | 63.64 | C | O |
| ATOM | 10615 | N | PHE I | 505 | −10.813 | 12.029 | −50.922 | 1.00 | 58.17 | C | N |
| ATOM | 10616 | CA | PHE I | 505 | −10.485 | 11.505 | −52.240 | 1.00 | 56.56 | C | C |
| ATOM | 10617 | CB | PHE I | 505 | −9.180 | 10.698 | −52.186 | 1.00 | 20.00 | C | C |
| ATOM | 10618 | CG | PHE I | 505 | −7.971 | 11.517 | −51.817 | 1.00 | 20.00 | C | C |
| ATOM | 10619 | CD1 | PHE I | 505 | −7.307 | 12.270 | −52.773 | 1.00 | 20.00 | C | C |
| ATOM | 10620 | CE1 | PHE I | 505 | −6.230 | 13.063 | −52.426 | 1.00 | 20.00 | C | C |
| ATOM | 10621 | CZ | PHE I | 505 | −5.768 | 13.067 | −51.125 | 1.00 | 20.00 | C | C |
| ATOM | 10622 | CE2 | PHE I | 505 | −6.376 | 12.270 | −50.176 | 1.00 | 20.00 | C | C |
| ATOM | 10623 | CD2 | PHE I | 505 | −7.471 | 11.499 | −50.524 | 1.00 | 20.00 | C | C |
| ATOM | 10624 | C | PHE I | 505 | −11.625 | 10.637 | −52.769 | 1.00 | 60.12 | C | C |
| ATOM | 10625 | O | PHE I | 505 | −12.014 | 10.746 | −53.934 | 1.00 | 65.46 | C | O |
| ATOM | 10626 | N | ILE I | 506 | −12.214 | 9.844 | −51.875 | 1.00 | 56.10 | C | N |
| ATOM | 10627 | CA | ILE I | 506 | −13.355 | 8.991 | −52.213 | 1.00 | 57.32 | C | C |
| ATOM | 10628 | CB | ILE I | 506 | −13.782 | 8.109 | −51.013 | 1.00 | 20.00 | C | C |
| ATOM | 10629 | CG1 | ILE I | 506 | −12.657 | 7.149 | −50.619 | 1.00 | 20.00 | C | C |
| ATOM | 10630 | CD1 | ILE I | 506 | −12.804 | 6.591 | −49.222 | 1.00 | 20.00 | C | C |
| ATOM | 10631 | CG2 | ILE I | 506 | −15.064 | 7.345 | −51.330 | 1.00 | 20.00 | C | C |
| ATOM | 10632 | C | ILE I | 506 | −14.556 | 9.818 | −52.669 | 1.00 | 67.33 | C | C |
| ATOM | 10633 | O | ILE I | 506 | −15.217 | 9.485 | −53.652 | 1.00 | 76.99 | C | O |
| ATOM | 10634 | N | ARG I | 507 | −14.855 | 10.878 | −51.925 | 1.00 | 67.87 | C | N |
| ATOM | 10635 | CA | ARG I | 507 | −15.936 | 11.788 | −52.287 | 1.00 | 62.11 | C | C |
| ATOM | 10636 | CB | ARG I | 507 | −16.087 | 12.898 | −51.240 | 1.00 | 20.00 | C | C |
| ATOM | 10637 | CG | ARG I | 507 | −16.795 | 12.450 | −49.963 | 1.00 | 20.00 | C | C |
| ATOM | 10638 | CD | ARG I | 507 | −16.736 | 13.504 | −48.861 | 1.00 | 20.00 | C | C |
| ATOM | 10639 | NE | ARG I | 507 | −17.140 | 12.957 | −47.566 | 1.00 | 20.00 | C | N |
| ATOM | 10640 | CZ | ARG I | 507 | −17.292 | 13.683 | −46.463 | 1.00 | 20.00 | C | C |
| ATOM | 10641 | NH1 | ARG I | 507 | −17.083 | 14.992 | −46.499 | 1.00 | 20.00 | C | N |
| ATOM | 10642 | NH2 | ARG I | 507 | −17.687 | 13.106 | −45.335 | 1.00 | 20.00 | C | N |
| ATOM | 10643 | C | ARG I | 507 | −15.685 | 12.391 | −53.659 | 1.00 | 67.30 | C | C |
| ATOM | 10644 | O | ARG I | 507 | −16.488 | 12.224 | −54.574 | 1.00 | 67.02 | C | O |
| ATOM | 10645 | N | LYS I | 508 | −14.518 | 13.001 | −53.823 | 1.00 | 66.83 | C | N |
| ATOM | 10646 | CA | LYS I | 508 | −14.125 | 13.546 | −55.112 | 1.00 | 68.12 | C | C |
| ATOM | 10647 | CB | LYS I | 508 | −12.693 | 14.086 | −55.052 | 1.00 | 20.00 | C | C |
| ATOM | 10648 | CG | LYS I | 508 | −12.522 | 15.286 | −54.117 | 1.00 | 20.00 | C | C |
| ATOM | 10649 | CD | LYS I | 508 | −11.072 | 15.750 | −54.046 | 1.00 | 20.00 | C | C |
| ATOM | 10650 | CE | LYS I | 508 | −10.912 | 16.933 | −53.098 | 1.00 | 20.00 | C | C |
| ATOM | 10651 | NZ | LYS I | 508 | −9.486 | 17.329 | −52.912 | 1.00 | 20.00 | C | N |
| ATOM | 10652 | C | LYS I | 508 | −14.275 | 12.499 | −56.215 | 1.00 | 65.22 | C | C |
| ATOM | 10653 | O | LYS I | 508 | −15.005 | 12.710 | −57.183 | 1.00 | 72.34 | C | O |
| ATOM | 10654 | N | SER I | 509 | −13.727 | 11.312 | −55.973 | 1.00 | 64.87 | C | N |
| ATOM | 10655 | CA | SER I | 509 | −13.783 | 10.230 | −56.949 | 1.00 | 56.47 | C | C |
| ATOM | 10656 | CB | SER I | 509 | −13.014 | 9.008 | −56.445 | 1.00 | 20.00 | C | C |
| ATOM | 10657 | OG | SER I | 509 | −13.561 | 8.528 | −55.232 | 1.00 | 20.00 | C | O |
| ATOM | 10658 | C | SER I | 509 | −15.220 | 9.847 | −57.299 | 1.00 | 56.96 | C | C |
| ATOM | 10659 | O | SER I | 509 | −15.537 | 9.599 | −58.461 | 1.00 | 65.57 | C | O |
| ATOM | 10660 | N | ASP I | 510 | −16.077 | 9.760 | −56.285 | 1.00 | 64.34 | C | N |
| ATOM | 10661 | CA | ASP I | 510 | −17.478 | 9.402 | −56.500 | 1.00 | 64.83 | C | C |
| ATOM | 10662 | CB | ASP I | 510 | −18.217 | 9.244 | −55.161 | 1.00 | 20.00 | C | C |
| ATOM | 10663 | CG | ASP I | 510 | −17.827 | 7.975 | −54.416 | 1.00 | 20.00 | C | C |
| ATOM | 10664 | OD1 | ASP I | 510 | −17.292 | 7.044 | −55.050 | 1.00 | 20.00 | C | O |
| ATOM | 10665 | OD2 | ASP I | 510 | −18.083 | 7.900 | −53.197 | 1.00 | 20.00 | C | O |
| ATOM | 10666 | C | ASP I | 510 | −18.173 | 10.458 | −57.356 | 1.00 | 62.11 | C | C |
| ATOM | 10667 | O | ASP I | 510 | −18.723 | 10.150 | −58.412 | 1.00 | 67.61 | C | O |
| ATOM | 10668 | N | GLU I | 511 | −18.078 | 11.713 | −56.928 | 1.00 | 64.95 | C | N |
| ATOM | 10669 | CA | GLU I | 511 | −18.673 | 12.822 | −57.664 | 1.00 | 60.64 | C | C |
| ATOM | 10670 | CB | GLU I | 511 | −18.175 | 14.162 | −57.117 | 1.00 | 20.00 | C | C |
| ATOM | 10671 | CG | GLU I | 511 | −18.700 | 14.509 | −55.736 | 1.00 | 20.00 | C | C |
| ATOM | 10672 | CD | GLU I | 511 | −18.061 | 15.767 | −55.172 | 1.00 | 20.00 | C | C |
| ATOM | 10673 | OE1 | GLU I | 511 | −17.166 | 16.329 | −55.841 | 1.00 | 20.00 | C | O |
| ATOM | 10674 | OE2 | GLU I | 511 | −18.506 | 16.237 | −54.102 | 1.00 | 20.00 | C | O |
| ATOM | 10675 | C | GLU I | 511 | −18.377 | 12.729 | −59.159 | 1.00 | 59.91 | C | C |
| ATOM | 10676 | O | GLU I | 511 | −19.245 | 13.008 | −59.983 | 1.00 | 55.45 | C | O |
| ATOM | 10677 | N | LEU I | 512 | −17.122 | 12.450 | −59.503 | 1.00 | 44.35 | C | N |
| ATOM | 10678 | CA | LEU I | 512 | −16.712 | 12.392 | −60.904 | 1.00 | 49.60 | C | C |
| ATOM | 10679 | CB | LEU I | 512 | −15.196 | 12.240 | −61.021 | 1.00 | 20.00 | C | C |
| ATOM | 10680 | CG | LEU I | 512 | −14.353 | 13.460 | −60.658 | 1.00 | 20.00 | C | C |
| ATOM | 10681 | CD1 | LEU I | 512 | −12.894 | 13.181 | −60.963 | 1.00 | 20.00 | C | C |
| ATOM | 10682 | CD2 | LEU I | 512 | −14.831 | 14.678 | −61.424 | 1.00 | 20.00 | C | C |
| ATOM | 10683 | C | LEU I | 512 | −17.396 | 11.239 | −61.621 | 1.00 | 58.00 | C | C |
| ATOM | 10684 | O | LEU I | 512 | −17.946 | 11.411 | −62.708 | 1.00 | 55.11 | C | O |
| ATOM | 10685 | N | LEU I | 513 | −17.306 | 10.051 | −61.036 | 1.00 | 57.91 | C | N |
| ATOM | 10686 | CA | LEU I | 513 | −17.901 | 8.870 | −61.636 | 1.00 | 57.49 | C | C |
| ATOM | 10687 | CB | LEU I | 513 | −17.632 | 7.629 | −60.774 | 1.00 | 20.00 | C | C |
| ATOM | 10688 | CG | LEU I | 513 | −16.169 | 7.179 | −60.678 | 1.00 | 20.00 | C | C |
| ATOM | 10689 | CD1 | LEU I | 513 | −15.992 | 6.055 | −59.670 | 1.00 | 20.00 | C | C |
| ATOM | 10690 | CD2 | LEU I | 513 | −15.637 | 6.768 | −62.042 | 1.00 | 20.00 | C | C |
| ATOM | 10691 | C | LEU I | 513 | −19.399 | 9.075 | −61.852 | 1.00 | 58.78 | C | C |
| ATOM | 10692 | O | LEU I | 513 | −19.913 | 8.812 | −62.940 | 1.00 | 59.88 | C | O |
| ATOM | 10693 | N | HIS I | 514 | −20.054 | 9.718 | −60.888 | 1.00 | 60.07 | C | N |
| ATOM | 10694 | CA | HIS I | 514 | −21.494 | 9.975 | −60.982 | 1.00 | 62.82 | C | C |
| ATOM | 10695 | CB | HIS I | 514 | −22.058 | 10.454 | −59.635 | 1.00 | 20.00 | C | C |
| ATOM | 10696 | CG | HIS I | 514 | −21.878 | 9.472 | −58.517 | 1.00 | 20.00 | C | C |
| ATOM | 10697 | ND1 | HIS I | 514 | −22.551 | 8.269 | −58.465 | 1.00 | 20.00 | C | N |
| ATOM | 10698 | CE1 | HIS I | 514 | −22.192 | 7.612 | −57.376 | 1.00 | 20.00 | C | C |
| ATOM | 10699 | NE2 | HIS I | 514 | −21.304 | 8.342 | −56.723 | 1.00 | 20.00 | C | N |
| ATOM | 10700 | CD2 | HIS I | 514 | −21.104 | 9.517 | −57.407 | 1.00 | 20.00 | C | C |
| ATOM | 10701 | C | HIS I | 514 | −21.799 | 10.989 | −62.087 | 1.00 | 65.10 | C | C |
| ATOM | 10702 | O | HIS I | 514 | −22.898 | 11.002 | −62.647 | 1.00 | 66.73 | C | O |
| ATOM | 10703 | N | ASN I | 515 | −20.795 | 11.787 | −62.441 | 1.00 | 63.45 | C | N |
| ATOM | 10704 | CA | ASN I | 515 | −20.932 | 12.782 | −63.499 | 1.00 | 56.53 | C | C |
| ATOM | 10705 | CB | ASN I | 515 | −19.903 | 13.909 | −63.317 | 1.00 | 20.00 | C | C |
| ATOM | 10706 | CG | ASN I | 515 | −20.243 | 14.838 | −62.159 | 1.00 | 20.00 | C | C |
| ATOM | 10707 | OD1 | ASN I | 515 | −21.400 | 14.948 | −61.756 | 1.00 | 20.00 | C | O |
| ATOM | 10708 | ND2 | ASN I | 515 | −19.238 | 15.544 | −61.652 | 1.00 | 20.00 | C | N |
| ATOM | 10709 | C | ASN I | 515 | −20.765 | 12.134 | −64.868 | 1.00 | 60.06 | C | C |
| ATOM | 10710 | O | ASN I | 515 | −21.518 | 12.422 | −65.799 | 1.00 | 66.48 | C | O |
| ATOM | 10711 | N | VAL I | 516 | −19.829 | 11.197 | −64.959 | 1.00 | 54.29 | C | N |
| ATOM | 10712 | CA | VAL I | 516 | −19.617 | 10.460 | −66.195 | 1.00 | 52.70 | C | C |
| ATOM | 10713 | CB | VAL I | 516 | −18.407 | 9.523 | −66.097 | 1.00 | 20.00 | C | C |
| ATOM | 10714 | CG1 | VAL I | 516 | −18.398 | 8.554 | −67.267 | 1.00 | 20.00 | C | C |
| ATOM | 10715 | CG2 | VAL I | 516 | −17.122 | 10.330 | −66.063 | 1.00 | 20.00 | C | C |
| ATOM | 10716 | C | VAL I | 516 | −20.850 | 9.645 | −66.551 | 1.00 | 60.10 | C | C |
| ATOM | 10717 | O | VAL I | 516 | −21.306 | 9.662 | −67.693 | 1.00 | 61.55 | C | O |
| ATOM | 10718 | N | ASN I | 517 | −21.432 | 8.998 | −65.549 | 1.00 | 69.10 | C | N |
| ATOM | 10719 | CA | ASN I | 517 | −22.648 | 8.224 | −65.749 | 1.00 | 67.75 | C | C |
| ATOM | 10720 | CB | ASN I | 517 | −23.135 | 7.652 | −64.421 | 1.00 | 20.00 | C | C |
| ATOM | 10721 | CG | ASN I | 517 | −22.254 | 6.530 | −63.914 | 1.00 | 20.00 | C | C |
| ATOM | 10722 | OD1 | ASN I | 517 | −21.467 | 5.954 | −64.667 | 1.00 | 20.00 | C | O |
| ATOM | 10723 | ND2 | ASN I | 517 | −22.368 | 6.226 | −62.627 | 1.00 | 20.00 | C | N |
| ATOM | 10724 | C | ASN I | 517 | −23.751 | 9.051 | −66.394 | 1.00 | 71.17 | C | C |
| ATOM | 10725 | O | ASN I | 517 | −24.535 | 8.544 | −67.196 | 1.00 | 66.42 | C | O |
| ATOM | 10726 | N | ALA I | 518 | −23.811 | 10.326 | −66.028 | 1.00 | 72.11 | C | N |
| ATOM | 10727 | CA | ALA I | 518 | −24.811 | 11.232 | −66.571 | 1.00 | 74.82 | C | C |
| ATOM | 10728 | CB | ALA I | 518 | −24.936 | 12.464 | −65.692 | 1.00 | 20.00 | C | C |
| ATOM | 10729 | C | ALA I | 518 | −24.471 | 11.628 | −68.004 | 1.00 | 70.11 | C | C |
| ATOM | 10730 | O | ALA I | 518 | −25.328 | 11.590 | −68.886 | 1.00 | 65.18 | C | O |
| ATOM | 10731 | N | GLY I | 519 | −23.212 | 11.989 | −68.230 | 1.00 | 56.07 | C | N |
| ATOM | 10732 | CA | GLY I | 519 | −22.709 | 12.224 | −69.577 | 1.00 | 53.01 | C | C |
| ATOM | 10733 | C | GLY I | 519 | −22.906 | 11.026 | −70.483 | 1.00 | 63.58 | C | C |
| ATOM | 10734 | O | GLY I | 519 | −23.221 | 11.174 | −71.663 | 1.00 | 69.35 | C | O |
| ATOM | 10735 | N | LYS I | 520 | −22.776 | 9.833 | −69.912 | 1.00 | 58.33 | C | N |
| ATOM | 10736 | CA | LYS I | 520 | −22.996 | 8.592 | −70.647 | 1.00 | 53.44 | C | C |
| ATOM | 10737 | CB | LYS I | 520 | −22.655 | 7.389 | −69.767 | 1.00 | 20.00 | C | C |
| ATOM | 10738 | CG | LYS I | 520 | −21.194 | 7.319 | −69.368 | 1.00 | 20.00 | C | C |
| ATOM | 10739 | CD | LYS I | 520 | −20.915 | 6.097 | −68.525 | 1.00 | 20.00 | C | C |
| ATOM | 10740 | CE | LYS I | 520 | −19.428 | 5.920 | −68.314 | 1.00 | 20.00 | C | C |
| ATOM | 10741 | NZ | LYS I | 520 | −19.148 | 4.767 | −67.417 | 1.00 | 20.00 | C | N |
| ATOM | 10742 | C | LYS I | 520 | −24.437 | 8.485 | −71.121 | 1.00 | 58.24 | C | C |
| ATOM | 10743 | O | LYS I | 520 | −24.714 | 7.920 | −72.179 | 1.00 | 48.60 | C | O |
| ATOM | 10744 | N | SER I | 521 | −25.354 | 8.971 | −70.293 | 1.00 | 67.19 | C | N |
| ATOM | 10745 | CA | SER I | 521 | −26.772 | 8.995 | −70.632 | 1.00 | 60.42 | C | C |
| ATOM | 10746 | CB | SER I | 521 | −27.588 | 9.481 | −69.436 | 1.00 | 20.00 | C | C |
| ATOM | 10747 | OG | SER I | 521 | −27.281 | 10.830 | −69.130 | 1.00 | 20.00 | C | O |
| ATOM | 10748 | C | SER I | 521 | −27.047 | 9.892 | −71.837 | 1.00 | 54.19 | C | C |
| ATOM | 10749 | O | SER I | 521 | −27.767 | 9.506 | −72.758 | 1.00 | 65.36 | C | O |
| ATOM | 10750 | N | THR I | 522 | −26.556 | 11.124 | −71.770 | 1.00 | 50.09 | C | N |
| ATOM | 10751 | CA | THR I | 522 | −26.650 | 12.048 | −72.892 | 1.00 | 57.59 | C | C |
| ATOM | 10752 | CB | THR I | 522 | −25.950 | 13.377 | −72.579 | 1.00 | 20.00 | C | C |
| ATOM | 10753 | OG1 | THR I | 522 | −24.538 | 13.159 | −72.466 | 1.00 | 20.00 | C | O |
| ATOM | 10754 | CG2 | THR I | 522 | −26.478 | 13.957 | −71.275 | 1.00 | 20.00 | C | C |
| ATOM | 10755 | C | THR I | 522 | −26.040 | 11.450 | −74.153 | 1.00 | 59.16 | C | C |
| ATOM | 10756 | O | THR I | 522 | −26.682 | 11.416 | −75.202 | 1.00 | 65.90 | C | O |
| ATOM | 10757 | N | THR I | 523 | −24.840 | 10.891 | −74.020 | 1.00 | 52.59 | C | N |
| ATOM | 10758 | CA | THR I | 523 | −24.151 | 10.267 | −75.148 | 1.00 | 42.19 | C | C |
| ATOM | 10759 | CB | THR I | 523 | −22.805 | 9.649 | −74.725 | 1.00 | 20.00 | C | C |
| ATOM | 10760 | OG1 | THR I | 523 | −23.027 | 8.645 | −73.726 | 1.00 | 20.00 | C | O |
| ATOM | 10761 | CG2 | THR I | 523 | −21.876 | 10.722 | −74.172 | 1.00 | 20.00 | C | C |
| ATOM | 10762 | C | THR I | 523 | −25.002 | 9.194 | −75.822 | 1.00 | 48.90 | C | C |
| ATOM | 10763 | O | THR I | 523 | −24.991 | 9.059 | −77.047 | 1.00 | 63.19 | C | O |
| ATOM | 10764 | N | ASN I | 524 | −25.694 | 8.395 | −75.018 | 1.00 | 52.51 | C | N |
| ATOM | 10765 | CA | ASN I | 524 | −26.597 | 7.386 | −75.553 | 1.00 | 57.54 | C | C |
| ATOM | 10766 | CB | ASN I | 524 | −27.089 | 6.458 | −74.444 | 1.00 | 20.00 | C | C |
| ATOM | 10767 | CG | ASN I | 524 | −26.002 | 5.533 | −73.941 | 1.00 | 20.00 | C | C |
| ATOM | 10768 | OD1 | ASN I | 524 | −25.000 | 5.316 | −74.620 | 1.00 | 20.00 | C | O |
| ATOM | 10769 | ND2 | ASN I | 524 | −26.153 | 5.050 | −72.714 | 1.00 | 20.00 | C | N |
| ATOM | 10770 | C | ASN I | 524 | −27.776 | 7.996 | −76.302 | 1.00 | 54.12 | C | C |
| ATOM | 10771 | O | ASN I | 524 | −28.076 | 7.601 | −77.428 | 1.00 | 61.31 | C | O |
| ATOM | 10772 | N | SER I | 525 | −28.372 | 9.027 | −75.714 | 1.00 | 48.97 | C | N |
| ATOM | 10773 | CA | SER I | 525 | −29.483 | 9.734 | −76.336 | 1.00 | 64.81 | C | C |
| ATOM | 10774 | CB | SER I | 525 | −29.930 | 10.885 | −75.441 | 1.00 | 61.22 | C | C |
| ATOM | 10775 | OG | SER I | 525 | −29.691 | 10.571 | −74.078 | 1.00 | 85.90 | C | O |
| ATOM | 10776 | C | SER I | 525 | −29.114 | 10.265 | −77.713 | 1.00 | 69.67 | C | C |
| ATOM | 10777 | O | SER I | 525 | −29.974 | 10.417 | −78.580 | 1.00 | 72.60 | C | O |
| ATOM | 10778 | N | LYS I | 526 | −27.853 | 10.647 | −77.872 | 1.00 | 66.06 | C | N |
| ATOM | 10779 | CA | LYS I | 526 | −27.365 | 11.130 | −79.154 | 1.00 | 63.67 | C | C |
| ATOM | 10780 | CB | LYS I | 526 | −26.043 | 11.874 | −78.980 | 1.00 | 70.55 | C | C |
| ATOM | 10781 | CG | LYS I | 526 | −26.196 | 13.332 | −78.608 | 1.00 | 75.29 | C | C |
| ATOM | 10782 | CD | LYS I | 526 | −24.898 | 13.870 | −78.032 | 1.00 | 85.03 | C | C |
| ATOM | 10783 | CE | LYS I | 526 | −25.144 | 15.017 | −77.067 | 1.00 | 82.65 | C | C |
| ATOM | 10784 | NZ | LYS I | 526 | −24.040 | 15.141 | −76.075 | 1.00 | 73.89 | C | N |
| ATOM | 10785 | C | LYS I | 526 | −27.182 | 9.975 | −80.124 | 1.00 | 55.36 | C | C |
| ATOM | 10786 | O | LYS I | 526 | −27.591 | 10.050 | −81.283 | 1.00 | 70.69 | C | O |
| ATOM | 10787 | N | ILE I | 527 | −26.593 | 8.892 | −79.634 | 1.00 | 57.52 | C | N |
| ATOM | 10788 | CA | ILE I | 527 | −26.355 | 7.723 | −80.461 | 1.00 | 58.48 | C | C |
| ATOM | 10789 | CB | ILE I | 527 | −25.712 | 6.589 | −79.650 | 1.00 | 57.00 | C | C |
| ATOM | 10790 | CG1 | ILE I | 527 | −24.414 | 7.071 | −78.993 | 1.00 | 49.07 | C | C |
| ATOM | 10791 | CD1 | ILE I | 527 | −23.516 | 7.882 | −79.906 | 1.00 | 46.18 | C | C |
| ATOM | 10792 | CG2 | ILE I | 527 | −25.469 | 5.370 | −80.530 | 1.00 | 52.63 | C | C |
| ATOM | 10793 | C | ILE I | 527 | −27.649 | 7.230 | −81.097 | 1.00 | 65.20 | C | C |
| ATOM | 10794 | O | ILE I | 527 | −27.705 | 7.000 | −82.304 | 1.00 | 66.48 | C | O |
| ATOM | 10795 | N | TYR I | 528 | −28.705 | 7.141 | −80.293 | 1.00 | 69.78 | C | N |
| ATOM | 10796 | CA | TYR I | 528 | −30.027 | 6.783 | −80.798 | 1.00 | 72.52 | C | C |
| ATOM | 10797 | CB | TYR I | 528 | −31.080 | 6.916 | −79.695 | 1.00 | 73.16 | C | C |
| ATOM | 10798 | CG | TYR I | 528 | −30.768 | 6.138 | −78.433 | 1.00 | 76.94 | C | C |
| ATOM | 10799 | CD1 | TYR I | 528 | −31.402 | 6.444 | −77.233 | 1.00 | 70.95 | C | C |
| ATOM | 10800 | CE1 | TYR I | 528 | −31.123 | 5.739 | −76.074 | 1.00 | 67.73 | C | C |
| ATOM | 10801 | CZ | TYR I | 528 | −30.200 | 4.712 | −76.103 | 1.00 | 71.89 | C | C |
| ATOM | 10802 | OH | TYR I | 528 | −29.927 | 4.009 | −74.954 | 1.00 | 75.44 | C | O |
| ATOM | 10803 | CE2 | TYR I | 528 | −29.534 | 4.407 | −77.273 | 1.00 | 77.03 | C | C |
| ATOM | 10804 | CD2 | TYR I | 528 | −29.818 | 5.120 | −78.431 | 1.00 | 77.84 | C | C |
| ATOM | 10805 | C | TYR I | 528 | −30.414 | 7.661 | −81.985 | 1.00 | 71.63 | C | C |
| ATOM | 10806 | O | TYR I | 528 | −30.720 | 7.162 | −83.069 | 1.00 | 84.20 | C | O |
| ATOM | 10807 | N | HIS I | 529 | −30.470 | 8.966 | −81.748 | 1.00 | 67.69 | C | N |
| ATOM | 10808 | CA | HIS I | 529 | −30.934 | 9.902 | −82.759 | 1.00 | 60.28 | C | C |
| ATOM | 10809 | CB | HIS I | 529 | −30.900 | 11.336 | −82.218 | 1.00 | 67.84 | C | C |
| ATOM | 10810 | CG | HIS I | 529 | −31.927 | 11.615 | −81.162 | 1.00 | 88.54 | C | C |
| ATOM | 10811 | ND1 | HIS I | 529 | −32.439 | 12.875 | −80.934 | 1.00 | 93.34 | C | N |
| ATOM | 10812 | CE1 | HIS I | 529 | −33.289 | 12.828 | −79.924 | 1.00 | 86.23 | C | C |
| ATOM | 10813 | NE2 | HIS I | 529 | −33.346 | 11.583 | −79.486 | 1.00 | 91.95 | C | N |
| ATOM | 10814 | CD2 | HIS I | 529 | −32.495 | 10.807 | −80.235 | 1.00 | 93.77 | C | C |
| ATOM | 10815 | C | HIS I | 529 | −30.094 | 9.785 | −84.029 | 1.00 | 63.30 | C | C |
| ATOM | 10816 | O | HIS I | 529 | −30.632 | 9.642 | −85.127 | 1.00 | 61.02 | C | O |
| ATOM | 10817 | N | ILE I | 530 | −28.775 | 9.752 | −83.867 | 1.00 | 59.79 | C | N |
| ATOM | 10818 | CA | ILE I | 530 | −27.886 | 9.457 | −84.984 | 1.00 | 45.70 | C | C |
| ATOM | 10819 | CB | ILE I | 530 | −26.413 | 9.365 | −84.536 | 1.00 | 40.57 | C | C |
| ATOM | 10820 | CG1 | ILE I | 530 | −25.854 | 10.764 | −84.270 | 1.00 | 42.86 | C | C |
| ATOM | 10821 | CD1 | ILE I | 530 | −24.376 | 10.902 | −84.570 | 1.00 | 51.01 | C | C |
| ATOM | 10822 | CG2 | ILE I | 530 | −25.575 | 8.662 | −85.596 | 1.00 | 35.31 | C | C |
| ATOM | 10823 | C | ILE I | 530 | −28.301 | 8.153 | −85.660 | 1.00 | 57.52 | C | C |
| ATOM | 10824 | O | ILE I | 530 | −28.637 | 8.136 | −86.845 | 1.00 | 71.18 | C | O |
| ATOM | 10825 | N | GLU I | 531 | −28.485 | 7.122 | −84.841 | 1.00 | 67.55 | C | N |
| ATOM | 10826 | CA | GLU I | 531 | −28.876 | 5.801 | −85.321 | 1.00 | 69.79 | C | C |
| ATOM | 10827 | CB | GLU I | 531 | −29.114 | 4.869 | −84.134 | 1.00 | 84.02 | C | C |
| ATOM | 10828 | CG | GLU I | 531 | −28.794 | 3.415 | −84.402 | 1.00 | 104.22 | C | C |
| ATOM | 10829 | CD | GLU I | 531 | −28.980 | 2.551 | −83.170 | 1.00 | 116.08 | C | C |
| ATOM | 10830 | OE1 | GLU I | 531 | −28.474 | 2.927 | −82.091 | 1.00 | 118.71 | C | O |
| ATOM | 10831 | OE2 | GLU I | 531 | −29.736 | 1.564 | −83.255 | 1.00 | 119.69 | C | O |
| ATOM | 10832 | C | GLU I | 531 | −30.145 | 5.890 | −86.151 | 1.00 | 64.87 | C | C |
| ATOM | 10833 | O | GLU I | 531 | −30.218 | 5.343 | −87.250 | 1.00 | 55.58 | C | O |
| ATOM | 10834 | N | ASN I | 532 | −31.177 | 6.489 | −85.566 | 1.00 | 65.70 | C | N |
| ATOM | 10835 | CA | ASN I | 532 | −32.459 | 6.657 | −86.240 | 1.00 | 64.16 | C | C |
| ATOM | 10836 | CB | ASN I | 532 | −33.475 | 7.303 | −85.296 | 1.00 | 60.58 | C | C |
| ATOM | 10837 | CG | ASN I | 532 | −34.859 | 7.396 | −85.905 | 1.00 | 67.75 | C | C |
| ATOM | 10838 | OD1 | ASN I | 532 | −35.480 | 6.381 | −86.221 | 1.00 | 53.59 | C | O |
| ATOM | 10839 | ND2 | ASN I | 532 | −35.352 | 8.618 | −86.072 | 1.00 | 61.81 | C | N |
| ATOM | 10840 | C | ASN I | 532 | −32.330 | 7.486 | −87.517 | 1.00 | 72.17 | C | C |
| ATOM | 10841 | O | ASN I | 532 | −32.999 | 7.220 | −88.517 | 1.00 | 85.75 | C | O |
| ATOM | 10842 | N | GLU I | 533 | −31.469 | 8.495 | −87.470 | 1.00 | 79.68 | C | N |
| ATOM | 10843 | CA | GLU I | 533 | −31.248 | 9.372 | −88.611 | 1.00 | 71.96 | C | C |
| ATOM | 10844 | CB | GLU I | 533 | −30.422 | 10.590 | −88.186 | 1.00 | 67.87 | C | C |
| ATOM | 10845 | CG | GLU I | 533 | −30.518 | 11.797 | −89.125 | 1.00 | 81.94 | C | C |
| ATOM | 10846 | CD | GLU I | 533 | −31.932 | 12.348 | −89.256 | 1.00 | 92.79 | C | C |
| ATOM | 10847 | OE1 | GLU I | 533 | −32.529 | 12.729 | −88.227 | 1.00 | 95.90 | C | O |
| ATOM | 10848 | OE2 | GLU I | 533 | −32.399 | 12.511 | −90.403 | 1.00 | 93.14 | C | O |
| ATOM | 10849 | C | GLU I | 533 | −30.546 | 8.622 | −89.738 | 1.00 | 68.54 | C | C |
| ATOM | 10850 | O | GLU I | 533 | −30.737 | 8.928 | −90.914 | 1.00 | 79.29 | C | O |
| ATOM | 10851 | N | ILE I | 534 | −29.742 | 7.631 | −89.367 | 1.00 | 69.50 | C | N |
| ATOM | 10852 | CA | ILE I | 534 | −29.047 | 6.796 | −90.342 | 1.00 | 72.55 | C | C |
| ATOM | 10853 | CB | ILE I | 534 | −27.786 | 6.137 | −89.729 | 1.00 | 79.55 | C | C |
| ATOM | 10854 | CG1 | ILE I | 534 | −26.779 | 7.205 | −89.298 | 1.00 | 80.41 | C | C |
| ATOM | 10855 | CD1 | ILE I | 534 | −26.687 | 8.374 | −90.254 | 1.00 | 89.38 | C | C |
| ATOM | 10856 | CG2 | ILE I | 534 | −27.138 | 5.186 | −90.724 | 1.00 | 78.63 | C | C |
| ATOM | 10857 | C | ILE I | 534 | −29.972 | 5.707 | −90.883 | 1.00 | 74.92 | C | C |
| ATOM | 10858 | O | ILE I | 534 | −29.725 | 5.139 | −91.948 | 1.00 | 81.01 | C | O |
| ATOM | 10859 | N | ALA I | 535 | −31.028 | 5.411 | −90.130 | 1.00 | 79.52 | C | N |
| ATOM | 10860 | CA | ALA I | 535 | −32.056 | 4.474 | −90.573 | 1.00 | 82.64 | C | C |
| ATOM | 10861 | CB | ALA I | 535 | −32.890 | 4.001 | −89.386 | 1.00 | 75.39 | C | C |
| ATOM | 10862 | C | ALA I | 535 | −32.956 | 5.093 | −91.645 | 1.00 | 81.39 | C | C |
| ATOM | 10863 | O | ALA I | 535 | −33.323 | 4.435 | −92.622 | 1.00 | 85.54 | C | O |
| ATOM | 10864 | N | ARG I | 536 | −33.274 | 6.373 | −91.474 | 1.00 | 78.41 | C | N |
| ATOM | 10865 | CA | ARG I | 536 | −34.100 | 7.098 | −92.435 | 1.00 | 83.27 | C | C |
| ATOM | 10866 | CB | ARG I | 536 | −34.664 | 8.372 | −91.804 | 1.00 | 84.58 | C | C |
| ATOM | 10867 | CG | ARG I | 536 | −35.623 | 8.123 | −90.651 | 1.00 | 103.24 | C | C |
| ATOM | 10868 | CD | ARG I | 536 | −36.750 | 9.145 | −90.630 | 1.00 | 114.72 | C | C |
| ATOM | 10869 | NE | ARG I | 536 | −36.250 | 10.513 | −90.521 | 1.00 | 117.80 | C | N |
| ATOM | 10870 | CZ | ARG I | 536 | −37.009 | 11.598 | −90.638 | 1.00 | 116.66 | C | C |
| ATOM | 10871 | NH1 | ARG I | 536 | −38.311 | 11.478 | −90.863 | 1.00 | 114.57 | C | N |
| ATOM | 10872 | NH2 | ARG I | 536 | −36.470 | 12.803 | −90.520 | 1.00 | 116.38 | C | N |
| ATOM | 10873 | C | ARG I | 536 | −33.323 | 7.440 | −93.703 | 1.00 | 82.20 | C | C |
| ATOM | 10874 | O | ARG I | 536 | −33.838 | 7.298 | −94.812 | 1.00 | 86.28 | C | O |
| ATOM | 10875 | N | ILE I | 537 | −32.114 | 7.964 | −93.532 | 1.00 | 77.08 | C | N |
| ATOM | 10876 | CA | ILE I | 537 | −31.219 | 8.194 | −94.659 | 1.00 | 66.79 | C | C |
| ATOM | 10877 | CB | ILE I | 537 | −29.908 | 8.850 | −94.204 | 1.00 | 64.76 | C | C |
| ATOM | 10878 | CG1 | ILE I | 537 | −30.176 | 10.251 | −93.649 | 1.00 | 66.92 | C | C |
| ATOM | 10879 | CD1 | ILE I | 537 | −29.017 | 10.829 | −92.869 | 1.00 | 70.83 | C | C |
| ATOM | 10880 | CG2 | ILE I | 537 | −28.926 | 8.905 | −95.354 | 1.00 | 51.02 | C | C |
| ATOM | 10881 | C | ILE I | 537 | −30.900 | 6.892 | −95.397 | 1.00 | 74.94 | C | C |
| ATOM | 10882 | O | ILE I | 537 | −30.628 | 6.898 | −96.599 | 1.00 | 97.16 | C | O |
| ATOM | 10883 | N | LYS I | 538 | −30.959 | 5.777 | −94.675 | 1.00 | 75.09 | C | N |
| ATOM | 10884 | CA | LYS I | 538 | −30.769 | 4.462 | −95.275 | 1.00 | 90.28 | C | C |
| ATOM | 10885 | CB | LYS I | 538 | −30.228 | 3.472 | −94.240 | 1.00 | 93.73 | C | C |
| ATOM | 10886 | CG | LYS I | 538 | −29.538 | 2.255 | −94.850 | 1.00 | 96.60 | C | C |
| ATOM | 10887 | CD | LYS I | 538 | −29.040 | 1.288 | −93.786 | 1.00 | 95.37 | C | C |
| ATOM | 10888 | CE | LYS I | 538 | −28.132 | 0.235 | −94.399 | 1.00 | 100.63 | C | C |
| ATOM | 10889 | NZ | LYS I | 538 | −27.652 | −0.753 | −93.397 | 1.00 | 99.58 | C | N |
| ATOM | 10890 | C | LYS I | 538 | −32.062 | 3.927 | −95.884 | 1.00 | 102.20 | C | C |
| ATOM | 10891 | O | LYS I | 538 | −32.083 | 2.839 | −96.461 | 1.00 | 112.11 | C | O |
| ATOM | 10892 | N | LYS I | 539 | −33.126 | 4.722 | −95.795 | 1.00 | 105.15 | C | N |
| ATOM | 10893 | CA | LYS I | 539 | −34.422 | 4.355 | −96.360 | 1.00 | 105.65 | C | C |
| ATOM | 10894 | CB | LYS I | 539 | −35.548 | 4.745 | −95.398 | 1.00 | 110.12 | C | C |
| ATOM | 10895 | CG | LYS I | 539 | −36.926 | 4.269 | −95.823 | 1.00 | 113.31 | C | C |
| ATOM | 10896 | CD | LYS I | 539 | −38.013 | 4.785 | −94.889 | 1.00 | 114.26 | C | C |
| ATOM | 10897 | CE | LYS I | 539 | −39.395 | 4.373 | −95.378 | 1.00 | 118.39 | C | C |
| ATOM | 10898 | NZ | LYS I | 539 | −40.449 | 4.593 | −94.352 | 1.00 | 121.77 | C | N |
| ATOM | 10899 | C | LYS I | 539 | −34.646 | 5.008 | −97.726 | 1.00 | 108.88 | C | C |
| ATOM | 10900 | O | LYS I | 539 | −35.717 | 4.876 | −98.321 | 1.00 | 110.77 | C | O |
| ATOM | 10901 | N | LEU I | 540 | −33.612 | 5.675 | −98.232 | 1.00 | 111.50 | C | N |
| ATOM | 10902 | CA | LEU I | 540 | −33.668 | 6.309 | −99.544 | 1.00 | 110.31 | C | C |
| ATOM | 10903 | CB | LEU I | 540 | −33.272 | 7.777 | −99.435 | 1.00 | 101.19 | C | C |
| ATOM | 10904 | CG | LEU I | 540 | −34.219 | 8.611 | −98.581 | 1.00 | 94.26 | C | C |
| ATOM | 10905 | CD1 | LEU I | 540 | −34.093 | 10.066 | −98.978 | 1.00 | 86.40 | C | C |
| ATOM | 10906 | CD2 | LEU I | 540 | −35.646 | 8.133 | −98.783 | 1.00 | 84.54 | C | C |
| ATOM | 10907 | C | LEU I | 540 | −32.738 | 5.601 | −100.513 | 1.00 | 118.37 | C | C |
| ATOM | 10908 | O | LEU I | 540 | −32.526 | 6.059 | −101.634 | 1.00 | 116.71 | C | O |
| ATOM | 10909 | N | ILE I | 541 | −32.163 | 4.493 | −100.056 | 1.00 | 130.80 | C | N |
| ATOM | 10910 | CA | ILE I | 541 | −31.273 | 3.686 | −100.882 | 1.00 | 140.48 | C | C |
| ATOM | 10911 | CB | ILE I | 541 | −29.821 | 4.210 | −100.840 | 1.00 | 138.99 | C | C |
| ATOM | 10912 | CG1 | ILE I | 541 | −29.770 | 5.686 | −101.240 | 1.00 | 133.30 | C | C |
| ATOM | 10913 | CD1 | ILE I | 541 | −30.650 | 6.027 | −102.420 | 1.00 | 136.26 | C | C |
| ATOM | 10914 | CG2 | ILE I | 541 | −28.924 | 3.372 | −101.744 | 1.00 | 136.12 | C | C |
| ATOM | 10915 | C | ILE I | 541 | −31.289 | 2.233 | −100.426 | 1.00 | 144.97 | C | C |
| ATOM | 10916 | O | ILE I | 541 | −30.938 | 1.333 | −101.188 | 1.00 | 145.83 | C | O |
| ATOM | 10917 | N | GLY I | 542 | −31.724 | 2.004 | −99.191 | 1.00 | 148.77 | C | N |
| ATOM | 10918 | CA | GLY I | 542 | −31.789 | 0.655 | −98.645 | 1.00 | 153.58 | C | C |
| ATOM | 10919 | C | GLY I | 542 | −33.208 | 0.136 | −98.534 | 1.00 | 160.45 | C | C |
| ATOM | 10920 | O | GLY I | 542 | −34.109 | 0.602 | −99.233 | 1.00 | 163.41 | C | O |
| ATOM | 10921 | N | GLU I | 543 | −33.412 | −0.799 | −97.611 | 1.00 | 163.83 | C | N |
| ATOM | 10922 | CA | GLU I | 543 | −34.718 | −1.409 | −97.392 | 1.00 | 165.52 | C | C |
| ATOM | 10923 | CB | GLU I | 543 | −34.576 | −2.673 | −96.538 | 1.00 | 158.94 | C | C |
| ATOM | 10928 | C | GLU I | 543 | −35.680 | −0.430 | −96.729 | 1.00 | 168.27 | C | C |
| ATOM | 10929 | O | GLU I | 543 | −35.373 | 0.753 | −96.584 | 1.00 | 167.37 | C | O |
| ATOM | 10930 | C1 | NAG J | 1535 | 24.849 | −25.592 | 34.903 | 1.00 | 72.96 | | C |
| ATOM | 10931 | C2 | NAG J | 1535 | 24.818 | −27.046 | 34.438 | 1.00 | 83.63 | | C |
| ATOM | 10932 | N2 | NAG J | 1535 | 23.774 | −27.757 | 35.160 | 1.00 | 85.81 | | N |
| ATOM | 10933 | C7 | NAG J | 1535 | 23.087 | −28.774 | 34.642 | 1.00 | 87.77 | | C |
| ATOM | 10934 | O7 | NAG J | 1535 | 23.376 | −29.307 | 33.573 | 1.00 | 89.34 | | O |
| ATOM | 10935 | C8 | NAG J | 1535 | 21.991 | −29.339 | 35.498 | 1.00 | 86.84 | | C |
| ATOM | 10936 | C3 | NAG J | 1535 | 26.183 | −27.682 | 34.695 | 1.00 | 85.86 | | C |
| ATOM | 10937 | O3 | NAG J | 1535 | 27.009 | −27.488 | 33.569 | 1.00 | 84.14 | | O |
| ATOM | 10938 | C4 | NAG J | 1535 | 26.823 | −27.025 | 35.913 | 1.00 | 88.88 | | C |
| ATOM | 10939 | O4 | NAG J | 1535 | 27.730 | −27.923 | 36.530 | 1.00 | 96.14 | | O |
| ATOM | 10940 | C5 | NAG J | 1535 | 25.692 | −26.643 | 36.863 | 1.00 | 85.57 | | C |
| ATOM | 10941 | C6 | NAG J | 1535 | 26.185 | −26.278 | 38.259 | 1.00 | 85.54 | | C |
| ATOM | 10942 | O6 | NAG J | 1535 | 27.307 | −25.431 | 38.159 | 1.00 | 85.75 | | O |
| ATOM | 10943 | O5 | NAG J | 1535 | 24.941 | −25.575 | 36.317 | 1.00 | 79.39 | | O |
| ATOM | 10944 | C1 | NAG J | 1536 | 29.076 | −27.393 | 36.512 | 1.00 | 101.26 | | C |
| ATOM | 10945 | C2 | NAG J | 1536 | 29.807 | −27.737 | 37.810 | 1.00 | 103.37 | | C |
| ATOM | 10946 | N2 | NAG J | 1536 | 30.222 | −26.531 | 38.504 | 1.00 | 104.36 | | N |
| ATOM | 10947 | C7 | NAG J | 1536 | 30.339 | −26.483 | 39.830 | 1.00 | 104.07 | | C |
| ATOM | 10948 | O7 | NAG J | 1536 | 30.062 | −27.429 | 40.566 | 1.00 | 103.82 | | O |
| ATOM | 10949 | C8 | NAG J | 1536 | 30.810 | −25.183 | 40.413 | 1.00 | 103.07 | | C |
| ATOM | 10950 | C3 | NAG J | 1536 | 31.013 | −28.638 | 37.568 | 1.00 | 104.12 | | C |
| ATOM | 10951 | O3 | NAG J | 1536 | 31.411 | −29.224 | 38.788 | 1.00 | 103.63 | | O |
| ATOM | 10952 | C4 | NAG J | 1536 | 30.689 | −29.737 | 36.565 | 1.00 | 104.68 | | C |
| ATOM | 10953 | O4 | NAG J | 1536 | 31.865 | −30.456 | 36.266 | 1.00 | 105.46 | | O |
| ATOM | 10954 | C5 | NAG J | 1536 | 30.104 | −29.166 | 35.277 | 1.00 | 104.39 | | C |
| ATOM | 10955 | C6 | NAG J | 1536 | 28.856 | −29.920 | 34.835 | 1.00 | 104.32 | | C |
| ATOM | 10956 | O6 | NAG J | 1536 | 28.929 | −30.178 | 33.450 | 1.00 | 104.22 | | O |
| ATOM | 10957 | O5 | NAG J | 1536 | 29.840 | −27.780 | 35.384 | 1.00 | 103.22 | | O |
| ATOM | 10958 | C1 | NAG J | 1545 | −15.969 | −4.075 | −43.033 | 1.00 | 56.85 | | C |
| ATOM | 10959 | C2 | NAG J | 1545 | −16.105 | −5.488 | −42.448 | 1.00 | 66.44 | | C |
| ATOM | 10960 | N2 | NAG J | 1545 | −14.794 | −6.111 | −42.415 | 1.00 | 69.47 | | N |
| ATOM | 10961 | C7 | NAG J | 1545 | −13.913 | −5.853 | −41.453 | 1.00 | 72.43 | | C |
| ATOM | 10962 | O7 | NAG J | 1545 | −14.178 | −5.152 | −40.481 | 1.00 | 73.31 | | O |
| ATOM | 10963 | C8 | NAG J | 1545 | −12.558 | −6.484 | −41.587 | 1.00 | 72.22 | | C |
| ATOM | 10964 | C3 | NAG J | 1545 | −17.084 | −6.379 | −43.216 | 1.00 | 68.81 | | C |
| ATOM | 10965 | O3 | NAG J | 1545 | −17.443 | −7.508 | −42.449 | 1.00 | 68.22 | | O |
| ATOM | 10966 | C4 | NAG J | 1545 | −18.314 | −5.557 | −43.561 | 1.00 | 71.19 | | C |
| ATOM | 10967 | O4 | NAG J | 1545 | −19.356 | −6.295 | −44.161 | 1.00 | 76.87 | | O |
| ATOM | 10968 | C5 | NAG J | 1545 | −17.827 | −4.416 | −44.429 | 1.00 | 67.79 | | C |
| ATOM | 10969 | C6 | NAG J | 1545 | −18.955 | −3.707 | −45.166 | 1.00 | 67.71 | | C |
| ATOM | 10970 | O6 | NAG J | 1545 | −19.545 | −2.754 | −44.311 | 1.00 | 67.33 | | O |
| ATOM | 10971 | O5 | NAG J | 1545 | −17.181 | −3.544 | −43.536 | 1.00 | 62.66 | | O |
| ATOM | 10972 | C1 | NAG J | 1546 | −20.251 | −6.690 | −43.101 | 1.00 | 83.18 | | C |
| ATOM | 10973 | C2 | NAG J | 1546 | −21.616 | −5.983 | −43.168 | 1.00 | 85.51 | | C |
| ATOM | 10974 | N2 | NAG J | 1546 | −21.766 | −5.147 | −41.990 | 1.00 | 84.12 | | N |
| ATOM | 10975 | C7 | NAG J | 1546 | −21.679 | −3.817 | −41.993 | 1.00 | 82.64 | | C |
| ATOM | 10976 | O7 | NAG J | 1546 | −21.413 | −3.148 | −42.990 | 1.00 | 81.92 | | O |
| ATOM | 10977 | C8 | NAG J | 1546 | −21.866 | −3.149 | −40.663 | 1.00 | 80.96 | | C |
| ATOM | 10978 | C3 | NAG J | 1546 | −22.846 | −6.884 | −43.261 | 1.00 | 87.47 | | C |
| ATOM | 10979 | O3 | NAG J | 1546 | −23.679 | −6.482 | −44.325 | 1.00 | 87.96 | | O |
| ATOM | 10980 | C4 | NAG J | 1546 | −22.495 | −8.356 | −43.377 | 1.00 | 87.61 | | C |
| ATOM | 10981 | O4 | NAG J | 1546 | −23.635 | −9.125 | −43.071 | 1.00 | 86.77 | | O |
| ATOM | 10982 | C5 | NAG J | 1546 | −21.399 | −8.665 | −42.371 | 1.00 | 87.12 | | C |
| ATOM | 10983 | C6 | NAG J | 1546 | −21.176 | −10.167 | −42.218 | 1.00 | 87.02 | | C |
| ATOM | 10984 | O6 | NAG J | 1546 | −20.698 | −10.439 | −40.920 | 1.00 | 86.44 | | O |
| ATOM | 10985 | O5 | NAG J | 1546 | −20.211 | −8.081 | −42.842 | 1.00 | 85.80 | | O |
| ATOM | 10986 | C1 | NAG J | 1525 | 19.621 | 34.609 | −15.280 | 1.00 | 72.96 | | C |
| ATOM | 10987 | C2 | NAG J | 1525 | 20.014 | 34.434 | −16.749 | 1.00 | 83.63 | | C |
| ATOM | 10988 | N2 | NAG J | 1525 | 21.459 | 34.306 | −16.817 | 1.00 | 85.81 | | N |
| ATOM | 10989 | C7 | NAG J | 1525 | 22.104 | 33.549 | −17.705 | 1.00 | 87.77 | | C |
| ATOM | 10990 | O7 | NAG J | 1525 | 21.559 | 33.036 | −18.682 | 1.00 | 89.34 | | O |
| ATOM | 10991 | C8 | NAG J | 1525 | 23.597 | 33.493 | −17.560 | 1.00 | 86.84 | | C |
| ATOM | 10992 | C3 | NAG J | 1525 | 19.546 | 35.624 | −17.590 | 1.00 | 85.86 | | C |
| ATOM | 10993 | O3 | NAG J | 1525 | 18.248 | 35.381 | −18.082 | 1.00 | 84.14 | | O |
| ATOM | 10994 | C4 | NAG J | 1525 | 19.548 | 36.890 | −16.742 | 1.00 | 88.88 | | C |
| ATOM | 10995 | O4 | NAG J | 1525 | 19.800 | 38.032 | −17.545 | 1.00 | 96.14 | | O |
| ATOM | 10996 | C5 | NAG J | 1525 | 20.649 | 36.717 | −15.706 | 1.00 | 85.57 | | C |
| ATOM | 10997 | C6 | NAG J | 1525 | 20.983 | 38.020 | −14.991 | 1.00 | 85.54 | | C |
| ATOM | 10998 | O6 | NAG J | 1525 | 19.792 | 38.678 | −14.623 | 1.00 | 85.75 | | O |
| ATOM | 10999 | O5 | NAG J | 1525 | 20.253 | 35.757 | −14.753 | 1.00 | 79.39 | | O |
| ATOM | 11000 | C1 | NAG J | 1526 | 18.686 | 38.953 | −17.492 | 1.00 | 101.26 | | C |
| ATOM | 11001 | C2 | NAG J | 1526 | 19.171 | 40.401 | −17.403 | 1.00 | 103.37 | | C |
| ATOM | 11002 | N2 | NAG J | 1526 | 18.643 | 41.051 | −16.216 | 1.00 | 104.36 | | N |
| ATOM | 11003 | C7 | NAG J | 1526 | 19.298 | 42.039 | −15.609 | 1.00 | 104.07 | | C |
| ATOM | 11004 | O7 | NAG J | 1526 | 20.406 | 42.432 | −15.972 | 1.00 | 103.82 | | O |
| ATOM | 11005 | C8 | NAG J | 1526 | 18.641 | 42.635 | −14.399 | 1.00 | 103.07 | | C |
| ATOM | 11006 | C3 | NAG J | 1526 | 18.781 | 41.209 | −18.636 | 1.00 | 104.12 | | C |
| ATOM | 11007 | O3 | NAG J | 1526 | 19.553 | 42.388 | −18.684 | 1.00 | 103.63 | | O |
| ATOM | 11008 | C4 | NAG J | 1526 | 19.007 | 40.409 | −19.911 | 1.00 | 104.68 | | C |
| ATOM | 11009 | O4 | NAG J | 1526 | 18.509 | 41.133 | −21.014 | 1.00 | 105.46 | | O |
| ATOM | 11010 | C5 | NAG J | 1526 | 18.320 | 39.050 | −19.846 | 1.00 | 104.39 | | C |
| ATOM | 11011 | C6 | NAG J | 1526 | 19.260 | 37.926 | −20.265 | 1.00 | 104.32 | | C |
| ATOM | 11012 | O6 | NAG J | 1526 | 18.612 | 37.103 | −21.209 | 1.00 | 104.22 | | O |
| ATOM | 11013 | O5 | NAG J | 1526 | 17.776 | 38.800 | −18.564 | 1.00 | 103.22 | | O |
| ATOM | 11014 | C1 | NAG K | 1535 | 41.248 | 18.154 | 31.115 | 1.00 | 72.96 | | C |
| ATOM | 11015 | C2 | NAG K | 1535 | 41.855 | 19.316 | 30.338 | 1.00 | 83.63 | | C |
| ATOM | 11016 | N2 | NAG K | 1535 | 43.088 | 18.851 | 29.728 | 1.00 | 85.81 | | N |
| ATOM | 11017 | C7 | NAG K | 1535 | 43.469 | 19.184 | 28.498 | 1.00 | 87.77 | | C |
| ATOM | 11018 | O7 | NAG K | 1535 | 42.908 | 20.051 | 27.830 | 1.00 | 89.34 | | O |
| ATOM | 11019 | C8 | NAG K | 1535 | 44.735 | 18.550 | 28.004 | 1.00 | 86.84 | | C |
| ATOM | 11020 | C3 | NAG K | 1535 | 42.118 | 20.499 | 31.270 | 1.00 | 85.86 | | C |
| ATOM | 11021 | O3 | NAG K | 1535 | 40.974 | 21.322 | 31.315 | 1.00 | 84.14 | | O |
| ATOM | 11022 | C4 | NAG K | 1535 | 42.448 | 20.012 | 32.677 | 1.00 | 88.88 | | C |
| ATOM | 11023 | O4 | NAG K | 1535 | 43.310 | 20.930 | 33.331 | 1.00 | 96.14 | | O |
| ATOM | 11024 | C5 | NAG K | 1535 | 43.123 | 18.655 | 32.535 | 1.00 | 85.57 | | C |
| ATOM | 11025 | C6 | NAG K | 1535 | 43.778 | 18.188 | 33.832 | 1.00 | 85.54 | | C |
| ATOM | 11026 | O6 | NAG K | 1535 | 43.027 | 18.627 | 34.942 | 1.00 | 85.75 | | O |
| ATOM | 11027 | O5 | NAG K | 1535 | 42.190 | 17.694 | 32.074 | 1.00 | 79.39 | | O |
| ATOM | 11028 | C1 | NAG K | 1536 | 42.680 | 21.487 | 34.509 | 1.00 | 101.26 | | C |
| ATOM | 11029 | C2 | NAG K | 1536 | 43.698 | 21.677 | 35.636 | 1.00 | 103.37 | | C |
| ATOM | 11030 | N2 | NAG K | 1536 | 43.310 | 20.938 | 36.825 | 1.00 | 104.36 | | N |
| ATOM | 11031 | C7 | NAG K | 1536 | 44.214 | 20.505 | 37.702 | 1.00 | 104.07 | | C |
| ATOM | 11032 | O7 | NAG K | 1536 | 45.425 | 20.656 | 37.545 | 1.00 | 103.82 | | O |
| ATOM | 11033 | C8 | NAG K | 1536 | 43.689 | 19.781 | 38.906 | 1.00 | 103.07 | | C |
| ATOM | 11034 | C3 | NAG K | 1536 | 43.900 | 23.146 | 35.994 | 1.00 | 104.12 | | C |
| ATOM | 11035 | O3 | NAG K | 1536 | 45.102 | 23.286 | 36.719 | 1.00 | 103.63 | | O |
| ATOM | 11036 | C4 | NAG K | 1536 | 43.966 | 24.019 | 34.748 | 1.00 | 104.68 | | C |
| ATOM | 11037 | O4 | NAG K | 1536 | 43.988 | 25.378 | 35.126 | 1.00 | 105.46 | | O |
| ATOM | 11038 | C5 | NAG K | 1536 | 42.782 | 23.769 | 33.821 | 1.00 | 104.39 | | C |
| ATOM | 11039 | C6 | NAG K | 1536 | 43.229 | 23.554 | 32.380 | 1.00 | 104.32 | | C |
| ATOM | 11040 | O6 | NAG K | 1536 | 42.483 | 24.396 | 31.530 | 1.00 | 104.22 | | O |
| ATOM | 11041 | O5 | NAG K | 1536 | 41.980 | 22.692 | 34.267 | 1.00 | 103.22 | | O |
| ATOM | 11042 | C1 | NAG K | 1545 | 2.615 | 1.983 | −49.407 | 1.00 | 56.85 | | C |
| ATOM | 11043 | C2 | NAG K | 1545 | 2.918 | 2.869 | −50.621 | 1.00 | 66.44 | | C |
| ATOM | 11044 | N2 | NAG K | 1545 | 1.785 | 3.731 | −50.894 | 1.00 | 69.47 | | N |
| ATOM | 11045 | C7 | NAG K | 1545 | 1.517 | 4.812 | −50.168 | 1.00 | 72.43 | | C |
| ATOM | 11046 | O7 | NAG K | 1545 | 2.154 | 5.121 | −49.163 | 1.00 | 73.31 | | O |
| ATOM | 11047 | C8 | NAG K | 1545 | 0.302 | 5.589 | −50.579 | 1.00 | 72.22 | | C |
| ATOM | 11048 | C3 | NAG K | 1545 | 3.247 | 2.088 | −51.890 | 1.00 | 68.81 | | C |
| ATOM | 11049 | O3 | NAG K | 1545 | 3.890 | 2.929 | −52.817 | 1.00 | 68.22 | | O |
| ATOM | 11050 | C4 | NAG K | 1545 | 4.146 | 0.927 | −51.513 | 1.00 | 71.19 | | C |
| ATOM | 11051 | O4 | NAG K | 1545 | 4.688 | 0.219 | −52.606 | 1.00 | 76.87 | | O |
| ATOM | 11052 | C5 | NAG K | 1545 | 3.310 | 0.066 | −50.596 | 1.00 | 67.79 | | C |
| ATOM | 11053 | C6 | NAG K | 1545 | 3.861 | −1.348 | −50.446 | 1.00 | 67.71 | | C |
| ATOM | 11054 | O6 | NAG K | 1545 | 4.940 | −1.351 | −49.538 | 1.00 | 67.33 | | O |
| ATOM | 11055 | O5 | NAG K | 1545 | 3.330 | 0.760 | −49.372 | 1.00 | 62.66 | | O |
| ATOM | 11056 | C1 | NAG K | 1546 | 5.996 | 0.777 | −52.858 | 1.00 | 83.18 | | C |
| ATOM | 11057 | C2 | NAG K | 1546 | 7.149 | −0.188 | −52.527 | 1.00 | 85.51 | | C |
| ATOM | 11058 | N2 | NAG K | 1546 | 7.911 | 0.336 | −51.403 | 1.00 | 84.12 | | N |
| ATOM | 11059 | C7 | NAG K | 1546 | 8.044 | −0.298 | −50.234 | 1.00 | 82.64 | | C |
| ATOM | 11060 | O7 | NAG K | 1546 | 7.581 | −1.414 | −50.007 | 1.00 | 81.92 | | O |
| ATOM | 11061 | C8 | NAG K | 1546 | 8.880 | 0.392 | −49.197 | 1.00 | 80.96 | | C |
| ATOM | 11062 | C3 | NAG K | 1546 | 8.093 | −0.539 | −53.684 | 1.00 | 87.47 | | C |
| ATOM | 11063 | O3 | NAG K | 1546 | 8.280 | −1.934 | −53.775 | 1.00 | 87.96 | | O |
| ATOM | 11064 | C4 | NAG K | 1546 | 7.594 | 0.007 | −55.013 | 1.00 | 87.61 | | C |
| ATOM | 11065 | O4 | NAG K | 1546 | 8.586 | −0.124 | −56.008 | 1.00 | 86.77 | | O |
| ATOM | 11066 | C5 | NAG K | 1546 | 7.269 | 1.469 | −54.777 | 1.00 | 87.12 | | C |
| ATOM | 11067 | C6 | NAG K | 1546 | 7.145 | 2.266 | −56.074 | 1.00 | 87.02 | | C |
| ATOM | 11068 | O6 | NAG K | 1546 | 7.707 | 3.548 | −55.896 | 1.00 | 86.44 | | O |
| ATOM | 11069 | O5 | NAG K | 1546 | 6.050 | 1.508 | −54.073 | 1.00 | 85.80 | | O |
| ATOM | 11070 | C1 | NAG K | 1525 | −29.090 | 1.326 | 0.431 | 1.00 | 56.85 | | C |
| ATOM | 11071 | C2 | NAG K | 1525 | −30.509 | 1.450 | 1.026 | 1.00 | 66.44 | | C |
| ATOM | 11072 | N2 | NAG K | 1525 | −30.921 | 0.195 | 1.623 | 1.00 | 69.47 | | N |
| ATOM | 11073 | C7 | NAG K | 1525 | −30.381 | −0.257 | 2.752 | 1.00 | 72.43 | | C |
| ATOM | 11074 | O7 | NAG K | 1525 | −29.481 | 0.332 | 3.347 | 1.00 | 73.31 | | O |
| ATOM | 11075 | C8 | NAG K | 1525 | −30.965 | −1.518 | 3.317 | 1.00 | 72.22 | | C |
| ATOM | 11076 | C3 | NAG K | 1525 | −31.621 | 1.959 | 0.102 | 1.00 | 68.81 | | C |
| ATOM | 11077 | O3 | NAG K | 1525 | −32.692 | 2.482 | 0.855 | 1.00 | 68.22 | | O |
| ATOM | 11078 | C4 | NAG K | 1525 | −31.056 | 3.022 | −0.817 | 1.00 | 71.19 | | C |
| ATOM | 11079 | O4 | NAG K | 1525 | −32.040 | 3.689 | −1.591 | 1.00 | 76.87 | | O |
| ATOM | 11080 | C5 | NAG K | 1525 | −30.031 | 2.269 | −1.642 | 1.00 | 67.79 | | C |
| ATOM | 11081 | C6 | NAG K | 1525 | −29.635 | 3.028 | −2.906 | 1.00 | 67.71 | | C |
| ATOM | 11082 | O6 | NAG K | 1525 | −28.857 | 4.152 | −2.561 | 1.00 | 67.33 | | O |
| ATOM | 11083 | O5 | NAG K | 1525 | −28.906 | 2.013 | −0.814 | 1.00 | 62.66 | | O |
| ATOM | 11084 | C1 | NAG K | 1526 | −32.430 | 4.903 | −0.902 | 1.00 | 83.18 | | C |
| ATOM | 11085 | C2 | NAG K | 1526 | −31.984 | 6.181 | −1.635 | 1.00 | 85.51 | | C |
| ATOM | 11086 | N2 | NAG K | 1526 | −30.942 | 6.832 | −0.859 | 1.00 | 84.12 | | N |
| ATOM | 11087 | C7 | NAG K | 1526 | −29.678 | 6.943 | −1.267 | 1.00 | 82.64 | | C |
| ATOM | 11088 | O7 | NAG K | 1526 | −29.276 | 6.528 | −2.353 | 1.00 | 81.92 | | O |
| ATOM | 11089 | C8 | NAG K | 1526 | −28.739 | 7.632 | −0.321 | 1.00 | 80.96 | | C |
| ATOM | 11090 | C3 | NAG K | 1526 | −33.070 | 7.210 | −1.965 | 1.00 | 87.47 | | C |
| ATOM | 11091 | O3 | NAG K | 1526 | −32.990 | 7.586 | −3.320 | 1.00 | 87.96 | | O |
| ATOM | 11092 | C4 | NAG K | 1526 | −34.468 | 6.712 | −1.644 | 1.00 | 87.61 | | C |
| ATOM | 11093 | O4 | NAG K | 1526 | −35.385 | 7.783 | −1.653 | 1.00 | 86.77 | | O |
| ATOM | 11094 | C5 | NAG K | 1526 | −34.395 | 6.107 | −0.257 | 1.00 | 87.12 | | C |
| ATOM | 11095 | C6 | NAG K | 1526 | −35.779 | 5.918 | 0.354 | 1.00 | 87.02 | | C |
| ATOM | 11096 | O6 | NAG K | 1526 | −35.724 | 6.194 | 1.736 | 1.00 | 86.44 | | O |
| ATOM | 11097 | O5 | NAG K | 1526 | −33.754 | 4.862 | −0.395 | 1.00 | 85.80 | | O |
| ATOM | 11098 | C1 | NAG L | 1535 | −2.070 | 11.341 | 45.870 | 1.00 | 72.96 | | C |
| ATOM | 11099 | C2 | NAG L | 1535 | −3.565 | 11.622 | 45.740 | 1.00 | 83.63 | | C |
| ATOM | 11100 | N2 | NAG L | 1535 | −3.793 | 13.060 | 45.821 | 1.00 | 85.81 | | N |
| ATOM | 11101 | C7 | NAG L | 1535 | −4.706 | 13.721 | 45.104 | 1.00 | 87.77 | | C |
| ATOM | 11102 | O7 | NAG L | 1535 | −5.570 | 13.168 | 44.424 | 1.00 | 89.34 | | O |
| ATOM | 11103 | C8 | NAG L | 1535 | −4.727 | 15.213 | 45.270 | 1.00 | 86.84 | | C |
| ATOM | 11104 | C3 | NAG L | 1535 | −4.303 | 10.883 | 46.855 | 1.00 | 85.86 | | C |
| ATOM | 11105 | O3 | NAG L | 1535 | −4.575 | 9.563 | 46.441 | 1.00 | 84.14 | | O |
| ATOM | 11106 | C4 | NAG L | 1535 | −3.441 | 10.862 | 48.115 | 1.00 | 88.88 | | C |
| ATOM | 11107 | O4 | NAG L | 1535 | −4.260 | 10.849 | 49.273 | 1.00 | 96.14 | | O |
| ATOM | 11108 | C5 | NAG L | 1535 | −2.549 | 12.100 | 48.077 | 1.00 | 85.57 | | C |
| ATOM | 11109 | C6 | NAG L | 1535 | −1.872 | 12.404 | 49.410 | 1.00 | 85.54 | | C |
| ATOM | 11110 | O6 | NAG L | 1535 | −1.282 | 11.235 | 49.931 | 1.00 | 85.75 | | O |
| ATOM | 11111 | O5 | NAG L | 1535 | −1.585 | 11.956 | 47.052 | 1.00 | 79.39 | | O |
| ATOM | 11112 | C1 | NAG L | 1536 | −4.061 | 9.626 | 50.020 | 1.00 | 101.26 | | C |
| ATOM | 11113 | C2 | NAG L | 1536 | −4.102 | 9.893 | 51.524 | 1.00 | 103.37 | | C |
| ATOM | 11114 | N2 | NAG L | 1536 | −2.863 | 9.461 | 52.140 | 1.00 | 104.36 | | N |
| ATOM | 11115 | C7 | NAG L | 1536 | −2.405 | 10.023 | 53.252 | 1.00 | 104.07 | | C |
| ATOM | 11116 | O7 | NAG L | 1536 | −2.958 | 10.972 | 53.807 | 1.00 | 103.82 | | O |
| ATOM | 11117 | C8 | NAG L | 1536 | −1.111 | 9.476 | 53.773 | 1.00 | 103.07 | | C |
| ATOM | 11118 | C3 | NAG L | 1536 | −5.265 | 9.179 | 52.199 | 1.00 | 104.12 | | C |
| ATOM | 11119 | O3 | NAG L | 1536 | −5.486 | 9.747 | 53.470 | 1.00 | 103.63 | | O |
| ATOM | 11120 | C4 | NAG L | 1536 | −6.532 | 9.306 | 51.370 | 1.00 | 104.68 | | C |
| ATOM | 11121 | O4 | NAG L | 1536 | −7.534 | 8.514 | 51.963 | 1.00 | 105.46 | | O |
| ATOM | 11122 | C5 | NAG L | 1536 | −6.309 | 8.841 | 49.937 | 1.00 | 104.39 | | C |
| ATOM | 11123 | C6 | NAG L | 1536 | −6.864 | 9.838 | 48.932 | 1.00 | 104.32 | | C |
| ATOM | 11124 | O6 | NAG L | 1536 | −7.576 | 9.132 | 47.943 | 1.00 | 104.22 | | O |
| ATOM | 11125 | O5 | NAG L | 1536 | −4.947 | 8.581 | 49.669 | 1.00 | 103.22 | | O |
| ATOM | 11126 | C1 | NAG L | 1545 | −11.652 | 16.132 | −42.376 | 1.00 | 56.85 | | C |
| ATOM | 11127 | C2 | NAG L | 1545 | −11.811 | 17.208 | −43.449 | 1.00 | 66.44 | | C |
| ATOM | 11128 | N2 | NAG L | 1545 | −10.485 | 17.531 | −43.934 | 1.00 | 69.47 | | N |
| ATOM | 11129 | C7 | NAG L | 1545 | −9.803 | 16.702 | −44.718 | 1.00 | 72.43 | | C |
| ATOM | 11130 | O7 | NAG L | 1545 | −10.291 | 15.689 | −45.219 | 1.00 | 73.31 | | O |
| ATOM | 11131 | C8 | NAG L | 1545 | −8.341 | 16.997 | −44.865 | 1.00 | 72.22 | | C |
| ATOM | 11132 | C3 | NAG L | 1545 | −12.475 | 18.463 | −42.898 | 1.00 | 68.81 | | C |
| ATOM | 11133 | O3 | NAG L | 1545 | −12.890 | 19.301 | −43.953 | 1.00 | 68.22 | | O |
| ATOM | 11134 | C4 | NAG L | 1545 | −13.667 | 18.017 | −42.071 | 1.00 | 71.19 | | C |
| ATOM | 11135 | O4 | NAG L | 1545 | −14.509 | 19.077 | −41.672 | 1.00 | 76.87 | | O |
| ATOM | 11136 | C5 | NAG L | 1545 | −13.113 | 17.218 | −40.907 | 1.00 | 67.79 | | C |
| ATOM | 11137 | C6 | NAG L | 1545 | −14.125 | 17.020 | −39.786 | 1.00 | 67.71 | | C |
| ATOM | 11138 | O6 | NAG L | 1545 | −15.086 | 16.069 | −40.180 | 1.00 | 67.33 | | O |
| ATOM | 11139 | O5 | NAG L | 1545 | −12.727 | 15.986 | −41.467 | 1.00 | 62.66 | | O |
| ATOM | 11140 | C1 | NAG L | 1546 | −15.574 | 19.184 | −42.644 | 1.00 | 83.18 | | C |
| ATOM | 11141 | C2 | NAG L | 1546 | −16.945 | 18.806 | −42.055 | 1.00 | 85.51 | | C |
| ATOM | 11142 | N2 | NAG L | 1546 | −17.436 | 17.613 | −42.723 | 1.00 | 84.12 | | N |
| ATOM | 11143 | C7 | NAG L | 1546 | −17.522 | 16.433 | −42.114 | 1.00 | 82.64 | | C |
| ATOM | 11144 | O7 | NAG L | 1546 | −17.214 | 16.256 | −40.937 | 1.00 | 81.92 | | O |
| ATOM | 11145 | C8 | NAG L | 1546 | −18.068 | 15.302 | −42.933 | 1.00 | 80.96 | | C |
| ATOM | 11146 | C3 | NAG L | 1546 | −18.033 | 19.883 | −42.093 | 1.00 | 87.47 | | C |
| ATOM | 11147 | O3 | NAG L | 1546 | −18.679 | 19.995 | −40.846 | 1.00 | 87.96 | | O |
| ATOM | 11148 | C4 | NAG L | 1546 | −17.484 | 21.228 | −42.533 | 1.00 | 87.61 | | C |
| ATOM | 11149 | O4 | NAG L | 1546 | −18.538 | 22.132 | −42.784 | 1.00 | 86.77 | | O |
| ATOM | 11150 | C5 | NAG L | 1546 | −16.723 | 20.935 | −43.810 | 1.00 | 87.12 | | C |
| ATOM | 11151 | C6 | NAG L | 1546 | −16.525 | 22.183 | −44.663 | 1.00 | 87.02 | | C |
| ATOM | 11152 | O6 | NAG L | 1546 | −17.041 | 21.939 | −45.954 | 1.00 | 86.44 | | O |
| ATOM | 11153 | O5 | NAG L | 1546 | −15.492 | 20.356 | −43.444 | 1.00 | 85.80 | | O |
| ATOM | 11154 | C1 | NAG L | 1525 | 21.793 | −26.723 | −18.244 | 1.00 | 56.85 | | C |
| ATOM | 11155 | C2 | NAG L | 1525 | 22.365 | −27.060 | −19.634 | 1.00 | 66.44 | | C |
| ATOM | 11156 | N2 | NAG L | 1525 | 22.402 | −25.862 | −20.460 | 1.00 | 69.47 | | N |
| ATOM | 11157 | C7 | NAG L | 1525 | 23.351 | −24.934 | −20.342 | 1.00 | 72.43 | | C |
| ATOM | 11158 | O7 | NAG L | 1525 | 24.279 | −25.026 | −19.540 | 1.00 | 73.31 | | O |
| ATOM | 11159 | C8 | NAG L | 1525 | 23.280 | −23.766 | −21.282 | 1.00 | 72.22 | | C |
| ATOM | 11160 | C3 | NAG L | 1525 | 21.624 | −28.181 | −20.369 | 1.00 | 68.81 | | C |
| ATOM | 11161 | O3 | NAG L | 1525 | 22.407 | −28.687 | −21.427 | 1.00 | 68.22 | | O |
| ATOM | 11162 | C4 | NAG L | 1525 | 21.308 | −29.275 | −19.364 | 1.00 | 71.19 | | C |
| ATOM | 11163 | O4 | NAG L | 1525 | 20.761 | −30.454 | −19.918 | 1.00 | 76.87 | | O |
| ATOM | 11164 | C5 | NAG L | 1525 | 20.389 | −28.631 | −18.348 | 1.00 | 67.79 | | C |
| ATOM | 11165 | C6 | NAG L | 1525 | 19.627 | −29.660 | −17.520 | 1.00 | 67.71 | | C |
| ATOM | 11166 | O6 | NAG L | 1525 | 20.442 | −30.144 | −16.478 | 1.00 | 67.33 | | O |
| ATOM | 11167 | O5 | NAG L | 1525 | 21.225 | −27.824 | −17.551 | 1.00 | 62.66 | | O |
| ATOM | 11168 | C1 | NAG L | 1526 | 21.846 | −31.384 | −20.124 | 1.00 | 83.18 | | C |
| ATOM | 11169 | C2 | NAG L | 1526 | 21.792 | −32.604 | −19.182 | 1.00 | 85.51 | | C |
| ATOM | 11170 | N2 | NAG L | 1526 | 22.928 | −32.540 | −18.274 | 1.00 | 84.12 | | N |
| ATOM | 11171 | C7 | NAG L | 1526 | 22.851 | −32.639 | −16.945 | 1.00 | 82.64 | | C |
| ATOM | 11172 | O7 | NAG L | 1526 | 21.797 | −32.785 | −16.328 | 1.00 | 81.92 | | O |
| ATOM | 11173 | C8 | NAG L | 1526 | 24.156 | −32.563 | −16.207 | 1.00 | 80.96 | | C |
| ATOM | 11174 | C3 | NAG L | 1526 | 21.773 | −33.981 | −19.854 | 1.00 | 87.47 | | C |
| ATOM | 11175 | O3 | NAG L | 1526 | 20.751 | −34.790 | −19.316 | 1.00 | 87.96 | | O |
| ATOM | 11176 | C4 | NAG L | 1526 | 21.648 | −33.901 | −21.365 | 1.00 | 87.61 | | C |
| ATOM | 11177 | O4 | NAG L | 1526 | 21.965 | −35.150 | −21.938 | 1.00 | 86.77 | | O |
| ATOM | 11178 | C5 | NAG L | 1526 | 22.647 | −32.860 | −21.827 | 1.00 | 87.12 | | C |
| ATOM | 11179 | C6 | NAG L | 1526 | 22.903 | −32.944 | −23.327 | 1.00 | 87.02 | | C |
| ATOM | 11180 | O6 | NAG L | 1526 | 24.294 | −32.969 | −23.563 | 1.00 | 86.44 | | O |
| ATOM | 11181 | O5 | NAG L | 1526 | 22.108 | −31.604 | −21.499 | 1.00 | 85.80 | | O |
| END | | | | | | | | | | | |

1.-12. (canceled)

13. A nucleic acid encoding a respiratory syncytial virus (RSV)—F protein comprising a substitution of an amino acid residue in a helix α1 corresponding to amino acid residues 145 to 157 of SEQ ID NO: 1 or 2 for a first cysteine residue and a substitution of an amino acid residue in a domain II (DII) corresponding to amino acid residues 400 to 460 of SEQ ID NO: 1 or 2 for a second cysteine residue.

14. The nucleic acid encoding the RSV-F protein of claim 13, wherein the RSV-F protein does not comprise a cytoplasmic tail.

15. The nucleic acid encoding the RSV-F protein of claim 13, wherein the RSV-F protein comprises a substitution of an amino acid residue in a helix α1 corresponding to amino acid residues 145 to 157 of SEQ ID NO: 1 or 2 for a first cysteine residue and a substitution of an amino acid residue in a domain II (DII) corresponding to amino acid residues 400 to 460 of SEQ ID NO: 1.

16. The nucleic acid encoding the RSV-F protein of claim 13, wherein the RSV-F protein comprises a substitution of an amino acid residue in a helix α1 corresponding to amino acid residues 145 to 157 of SEQ ID NO: 1 or 2 for a first cysteine residue and a substitution of an amino acid residue in a domain II (DII) corresponding to amino acid residues 400 to 460 of SEQ ID NO: 2.

17. The nucleic acid encoding the RSV-F protein of claim 13, wherein the RSV-F protein comprises a disulfide bond between the first cysteine residue and the second cysteine residue.

18. The nucleic acid encoding the RSV-F protein of claim 13, wherein the RSV-F protein does not comprise a p27 region.

19. The nucleic acid encoding the RSV-F protein of claim 13, wherein the RSV-F protein does not comprise a fusion peptide.

20. The nucleic acid encoding the RSV-F protein of claim 13, wherein the RSV-F protein does not comprise a signal peptide.

21. The nucleic acid encoding the RSV-F protein of claim 13, wherein the RSV-F protein comprises a disulfide bond between the first cysteine residue and the second cysteine residue, wherein the RSV-F protein does not comprise a p27 region, wherein the RSV-F protein does not comprise a fusion peptide, wherein the RSV-F protein does not comprise a cytoplasmic tail, and wherein the RSV-F protein does not comprise a signal peptide.

22. A vector comprising the nucleic acid of claim 13.

23. A vector comprising the nucleic acid of claim 14.

24. A vector comprising the nucleic acid of claim 15.

25. A vector comprising the nucleic acid of claim 16.

26. A vector comprising the nucleic acid of claim 17.

27. A vector comprising the nucleic acid of claim 18.

28. A vector comprising the nucleic acid of claim 19.

29. A vector comprising the nucleic acid of claim 20.

30. A vector comprising the nucleic acid of claim 21.

31. A host cell comprising the vector of claim 22.

32. A host cell comprising the vector of claim 23.

33. A host cell comprising the vector of claim 24.

34. A host cell comprising the vector of claim 25.

35. A host cell comprising the vector of claim 26.

36. A host cell comprising the vector of claim 27.

37. A host cell comprising the vector of claim 28.

38. A host cell comprising the vector of claim 29.

39. A host cell comprising the vector of claim 30.


SEQUENCE LISTING (41 sequences)

SEQ ID NO: 1 - protein, 574 aa, Human respiratory syncytial virus
  REGION 1..574: note = Human respiratory syncytial virus A2
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 2 - protein, 574 aa, Human respiratory syncytial virus
  REGION 1..574: note = Human respiratory syncytial virus subgroup B/strain 18537
MELLIHRSSAIFLTLAVNALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 3 - protein, 13 aa, synthetic construct
  REGION 1..13: note = Description of Artificial Sequence: Synthetic peptide
GGSAGSGHHHHHH

SEQ ID NO: 4 - protein, 537 aa, synthetic construct
  REGION 1..537: note = Description of Artificial Sequence: Synthetic polypeptide
MELLILKANAITTILTAVTFCFASGQNITEEFYQSTCSAVSKGYLSALRTGWYTSCITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGECNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNGGSAGSGHHHHHH

SEQ ID NO: 5 - protein, 537 aa, synthetic construct
  REGION 1..537: note = Description of Artificial Sequence: Synthetic polypeptide
MELLILKANAITTILTAVTFCFASGQNITEEFYQSTCSAVSKGYLSALRTGWYTSVCTIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTCKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNGGSAGSGHHHHHH

SEQ ID NO: 6 - protein, 537 aa, synthetic construct
  REGION 1..537: note = Description of Artificial Sequence: Synthetic polypeptide
MELLILKANAITTILTAVTFCFASGQNITEEFYQSTCSAVSKGYLSALRTGWYTSVICIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGECNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNGGSAGSGHHHHHH

SEQ ID NO: 7 - protein, 537 aa, synthetic construct
  REGION 1..537: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVCKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEECLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNGGSAGSGHHHHHH

SEQ ID NO: 8 - protein, 537 aa, synthetic construct
  REGION 1..537: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKICSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEECLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNGGSAGSGHHHHHH

SEQ ID NO: 9 - protein, 537 aa, synthetic construct
  REGION 1..537: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKICTSKTDVSSSVITSLGAIVSCYGKTKCTASNKNRGIIKTFSNGCDYVSNKGVDTVSVGNTLYYVNKQEGKSLYVKGEPIINFYDPLVCPSDEFDASISQVNEKINQSLAFIRKSDELLHNVNAGKSTTNGGSAGSGHHHHHH

SEQ ID NO: 10 - protein, 489 aa, synthetic construct
  REGION 1..489: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSGGSAGSGHHHHHH

SEQ ID NO: 11 - skipped

SEQ ID NO: 12 - protein, 6 aa, synthetic construct
  REGION 1..6: note = Description of Artificial Sequence: Synthetic 6xHis tag
HHHHHH

SEQ ID NO: 13 - protein, 534 aa, synthetic construct
  REGION 1..534: note = Description of Artificial Sequence: Synthetic polypeptide
MGSRSSTRIPVPLMLTVRVMLALSCVCPTSALDGRPLAAAGIVVTGDKAVNIYTSSQTGSIIIKLLPNMPKDKEACAKAPLEAYNRTLTTLLTPLGDSIRRIQESVTTSGGGKQGRLIGAIIGFLGFLLGVGSAIASGVAVSKVLHLEGEVNKIKSALLSTNKAVVSLSNGVSVLTSKVLDLKNYIDKQLLPIVNKQSCIKITQQVGVELNLYLTELTTVFGPQITSPALTQLTIQALYNLAGGNMDYLLTKLGVGNNQLSSLISSGLITGNPILYDSQTQLLGIQVTLPSVGNLNNMRATYLETLSVSTTKGFASALVPKVVTQVGSVIEELDTSYCIETDLDLYCTRIVTFPMSPGIYSCLSGNTSACMYSKTEGALTTPYMTLKGSVIANCKMTTCRCADPPGIISQNYGEAVSLIDRQSCNILSLDGITLRLSGEFDATYQKNISIQDSQVIVTGNLDISTELGNVNNSISNALDKLEESNSKLDKVEDKIEEILSKIYHIENEIARIKKLIGEAGGPLVPRGSHHHHHH

SEQ ID NO: 14 - protein, 4 aa, synthetic construct
  REGION 1..4: note = Description of Artificial Sequence: Synthetic peptide
RARK

SEQ ID NO: 15 - protein, 4 aa, synthetic construct
  REGION 1..4: note = Description of Artificial Sequence: Synthetic peptide
RARQ

SEQ ID NO: 16 - protein, 4 aa, synthetic construct
  REGION 1..4: note = Description of Artificial Sequence: Synthetic peptide
QAQN

SEQ ID NO: 17 - protein, 4 aa, synthetic construct
  REGION 1..4: note = Description of Artificial Sequence: Synthetic peptide
IEGR

SEQ ID NO: 18 - protein, 4 aa, synthetic construct
  REGION 1..4: note = Description of Artificial Sequence: Synthetic peptide
RKKK

SEQ ID NO: 19 - protein, 4 aa, synthetic construct
  REGION 1..4: note = Description of Artificial Sequence: Synthetic peptide
QNQN

SEQ ID NO: 20 - protein, 4 aa, synthetic construct
  REGION 1..4: note = Description of Artificial Sequence: Synthetic peptide
QQQR

SEQ ID NO: 21 - protein, 64 aa, synthetic construct
  REGION 1..64: note = Description of Artificial Sequence: Synthetic polypeptide
PLVFPSDEFDASISQVNEKINQSLAFIRKSDELLHNVNDKIEEILSKIYHIENEIARIKKLIGE

SEQ ID NO: 22 - protein, 60 aa, synthetic construct
  REGION 1..60: note = Description of Artificial Sequence: Synthetic polypeptide
PLVFPSDEFDASISQVNEKINQSLAFIRKSDELLHNVNEKFHQIEKEFSEVEGRIQDLEK

SEQ ID NO: 23 - protein, 38 aa, synthetic construct
  REGION 1..38: note = Description of Artificial Sequence: Synthetic polypeptide
PLVFPSDEFDASISQINEKINQILAFIRKIDELLHNIN

SEQ ID NO: 24 - protein, 67 aa, synthetic construct
  REGION 1..67: note = Description of Artificial Sequence: Synthetic polypeptide
PLVFPSDEFDASISQVNEKINQSLAFIRKSDELLHNVNGSGYIPEAPRDGQAYVRKDGEWVLLSTFL

SEQ ID NO: 25 - protein, 73 aa, synthetic construct
  REGION 1..73: note = Description of Artificial Sequence: Synthetic polypeptide
PLVFPSDEFDASISQVNEKINQSLAFIRKSDELLHNVNNKNDDKGSGYIPEAPRDGQAYVRKDGEWVLLSTFL

SEQ ID NO: 26 - protein, 4 aa, synthetic construct
  REGION 1..4: note = Description of Artificial Sequence: Synthetic peptide
LVPR

SEQ ID NO: 27 - protein, 5 aa, synthetic construct
  REGION 1..5: note = Description of Artificial Sequence: Synthetic peptide
DDDDK

SEQ ID NO: 28 - protein, 498 aa, synthetic construct
  REGION 1..498: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSGGSAGSGHHHHHH

SEQ ID NO: 29 - protein, 73 aa, synthetic construct
  REGION 1..73: note = Description of Artificial Sequence: Synthetic polypeptide
QNITEEFYQSTCSAVSKGYLSALRTGWYTSVITIELSNIKENKCNGTDAKVKLIKQELDKYKNAVTELQLLMQ

SEQ ID NO: 30 - protein, 189 aa, synthetic construct
  REGION 1..189: note = Description of Artificial Sequence: Synthetic polypeptide
FLGFLLGVGSAIASGVAVSKVLHLEGEVNKIKSALLSTNKAVVSLSNGVSVLTSKVLDLNNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPAYGVIDTPCWKLHTSPLCTTN

SEQ ID NO: 31 - protein, 136 aa, synthetic construct
  REGION 1..136: note = Description of Artificial Sequence: Synthetic polypeptide
SNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGK

SEQ ID NO: 32 - protein, 72 aa, synthetic construct
  REGION 1..72: note = Description of Artificial Sequence: Synthetic polypeptide
EPIINFYDPLVFPSDEFDASISQVNEKINQSLAFIRKSDELLHNVNAGKSTTNSKIYHIENEIARIKKLIGE

SEQ ID NO: 33 - protein, 499 aa, Human respiratory syncytial virus
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWFCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 34 - protein, 466 aa, Newcastle disease virus
SALDGRPLAAAGIVVTGDKAVNIYTSSQTGSIIIKLLPNMPKDKEACAKAPLEAYNRTLTTLLTPLGDSIRRIQESVTTSGGGKQGRLIGAIIGGVALGVATAAQITAASALIQANQNAANILRLKESIAATNEAVHEVTNGLSQLAVAVGKMQQFVNDQFNKTAQELDCIKITQQVGVELNLYLTELTTVFGPQITSPALTQLTIQALYNLAGGNMDYLLTKLGVGNNQLSSLISSGLITGNPILYDSQTQLLGIQVTLPSVGNLNNMRATYLETLSVSTTKGFASALVPKVVTQVGSVIEELDTSYCIETDLDLYCTRIVTFPMSPGIYSCLSGNTSACMYSKTEGALTTPYMTLKGSVIANCKMTTCRCADPPGIISQNYGEAVSLIDRQSCNILSLDGITLRLSGEFDATYQKNISIQDSQVIVTGNLDISTELGNVNNSISNALDKLEESNSKLDKVNVKL

SEQ ID NO: 35 - protein, 464 aa, synthetic construct
  REGION 1..464: note = Description of Artificial Sequence: Synthetic polypeptide
GSLDPAALMQIGVIPTNVRQLMYYTEASSAFIVVKLMPTIDSPISGCNITSISSYNATVTKLLQPIGENLETIRNQLIPTRRRRRFAGVVIGLAALGVATAAQVTAAVALVKANENAAAILNLKNAIQKTNAAVADVVQATQSLGTAVQAVQDHINSVVSPAITAANCKAQDAIIGSILNLYLTELTTIFHNQITNPALSPITIQALRILLGSTLPTVVEKSFNTQISAAELLSSGLLTGQIVGLDLTYMQMVIKIELPTLTVQPATQIIDLATISAFINNQEVMAQLPTRVMVTGSLIQAYPASQCTITPNTVYCRYNDAQVLSDDTMACLQGNLTRCTFSPVVGSFLTRFVLFDGIVYANCRSMLCKCMQPAAVILQPSSSPVTVIDMYKCVSLQLDNLRFTITQLANVTYNSTIKLESSQILSIDPLDISQNLAAVNKSLSDALQHLAQSDTYLSAITSAT

SEQ ID NO: 36 - protein, 471 aa, synthetic construct
  REGION 1..471: note = Description of Artificial Sequence: Synthetic polypeptide
CQIDITKLQHVGVLVNSPKGMKISQNFETRYLILSLIPKIEDSNSCGDQQIKQYKRLLDRLIIPLYDGLRLQKDVIVSNQESNENTDPRTKRFFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYRVDSISYNIQNREWYIPLPSHIMTKGAFLGGADVKECIEAFSSYICPSDPGFVLNHEMESCLSGNISQCPRTVVKSDIVPRYAFVNGGVVANCITTTCTCNGIGNRINQPPDQGVKIITHKECNTIGINGMLFNTNKEGTLAFYTPNDITLNNSVALDPIDISIELNKAKSDLEESKEWIRRSNQKLDSIGNWH

SEQ ID NO: 37 - protein, 5 aa, synthetic construct
  REGION 1..5: note = Description of Artificial Sequence: Synthetic peptide
IIKTF

SEQ ID NO: 38 - protein, 468 aa, synthetic construct
  REGION 1..468: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGGGSAGSGHHHHHH

SEQ ID NO: 39 - protein, 528 aa, synthetic construct
  REGION 1..528: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNSGITTTFFNGCDYVSNKGVDTVSVGNTLYYVNKQEGKSLYVKGEPIINFYDPLVFPSDEFDASISQVNEKINQSLAFIRKSDELLHNVNAGKSTTNGGSAGSGHHHHHH

SEQ ID NO: 40 - protein, 537 aa, synthetic construct; identical to SEQ ID NO: 5
  REGION 1..537: note = Description of Artificial Sequence: Synthetic polypeptide

SEQ ID NO: 41 - protein, 528 aa, synthetic construct
  REGION 1..528: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLIYDMPITIDQKMLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNSGITTTFFNGCDYVSNKGVDTVSVGNTLYYVNKQEGKSLYVKGEPIINFYDPLVFPSDEFDASISQVNEKINQSLAFIRKSDELLHNVNAGKSTTNGGSAGSGHHHHHH